(19) United States
(12) Patent Application Publication
(10) Pub. No.: US 2023/0074892 A1
(43) Pub. Date: Mar. 9, 2023

(54) BACTERIOPHAGE COMPOSITIONS AND USES THEREOF

(71) Applicant: YALE UNIVERSITY, New Haven, CT (US)

(72) Inventors: Paul Turner, New Haven, CT (US); Alita Burmeister, Hamden, CT (US); Benjamin Chan, Salt Lake City, UT (US); Kaitlyn Kortright, New Haven, CT (US); John Wertz, Cheshire, CT (US)

(21) Appl. No.: 17/609,225

(22) PCT Filed: May 7, 2020
(86) PCT No.: PCT/US20/31855

§ 371 Date: Nov. 5, 2021

Related U.S. Application Data
(60) Provisional application No. 63/017,369, filed on Apr. 29, 2020.
(60) Provisional application No. 62/844,515, filed on May 7, 2019.

(51) Int. Cl. A61K 35/76; C12N 7/00; A61P 31/04

(52) U.S. Cl. CPC A61K 35/76; A61P 31/04; C12N 7/00

(57) ABSTRACT

The present invention includes compositions and methods for increasing antibiotic sensitivity and/or decreasing virulence in bacteria.

### CROSS-REFERENCE TO RELATED APPLICATION

The present application is entitled to priority under 35 U.S.C. § 119(e) to U.S. Provisional Patent Application No. 62/844,515 filed May 7, 2019 and U.S. Provisional Patent Application No. 63/017,369, filed Apr. 29, 2020, each of which are hereby incorporated by reference in their entireties herein.

### STATEMENT REGARDING FEDERALLY SPONSORED RESEARCH OR DEVELOPMENT

This invention was made with government support under AI144345 awarded by National Institutes of Health and under 0939454 awarded by the National Science Foundation. The government has certain rights in the invention.

### BACKGROUND OF THE INVENTION

Widespread and inappropriate uses of chemical antibiotics have selected for multi-drug resistant (MDR) bacterial pathogens, presenting more frequently in human infections and contributing significantly to morbidity. Some bacteria even show evolved resistance to ‘drugs of last resort’, resulting in emergent strains that are pan-drug-resistant (PDR). One example is the Gram-negative bacterium, Pseudomonas aeruginosa, a prevalent opportunistic MDR pathogen that is poised to become a common PDR disease problem. Humans readily encounter P. aeruginosa, which thrives in both natural and artificial environments, varying from lakes and estuaries to hospitals and household sink drains. P. aeruginosa causes biofilm-mediated infections, including catheter associated urinary tract infections, ventilator associated pneumonia, and infections related to mechanical heart valves, stents, grafts and sutures (Cole, S. J., et al., Infection and Immunity 82, 2048-2058 (2014)). Individuals with cystic fibrosis, severe burns, surgical wounds and/or compromised immunity are particularly at risk for P. aeruginosa infections, especially acquired in hospitals.

P. aeruginosa is a ubiquitous Gram-negative, rod-shaped bacterium prevalent in natural and artificial environments (Remold, S. K., et al. Microb Ecol. 62(3), 505-17 (2011)). Adaptation to different habitats has allowed P. aeruginosa to persist in many human-associated environments, most notably in hospitals, where it is increasingly associated with nosocomial infections (Emori, T. G., et al., Clin Microbiol Rev. 6(4), 428-42 (1993)). These infections are difficult to manage, in part due to intrinsic antibiotic resistance resulting from decreased membrane permeability, active antibiotic efflux, and other chromosomally encoded enzymes. Further complicating the problem of P. aeruginosa infections are their ability to form biofilms, herein referred to as “P. aeruginosa biofilms” or “Pseudomonas aeruginosa biofilms”. Biofilm-mediated infections are notoriously difficult to manage, having seemingly much higher resistance to chemical antimicrobials (Stewart, P. S., et al., Lancet 358(9276), 135-08 (2001)) and often form following sub-lethal concentrations of antibiotics (Hoffman, L. R., et al., Nature 436(7054), 1171-5 (2005)). This elevated resistance may be due to exopolymeric substances in the biofilm matrix that slow diffusion of antibiotics and reduce effective concentrations. Furthermore, slow-growing cells present in the biofilm (e.g., persister cells) may have sufficiently reduced metabolisms to withstand bacteriostatic antibiotics that target metabolically active bacteria (Lewis, K. Biochemistry (Mosc). 70(2), 267-74 (2005)). As a result, biofilms may also act as a reservoir for the dissemination of infections throughout the body which could greatly prolong infection duration and severity. Prosthetic vascular graft infections are of significant concern due to the elevated mortality and morbidity rates. A common culprit, P. aeruginosa, presents a serious challenge due to its intrinsic antibiotic resistance and ability to form biofilms on prosthetic material.

Prosthetic vascular graft infections are catastrophic events which present serious challenges to surgeons and place heavy economic burdens on patients and the healthcare system. The reported incidence can vary from 0.6% to 9.5% depending on the site of the vascular graft (Kieffer, E., et al., J Vasc Surg. 33(4), 671-8 (2001); Schild, A. F., et al., J Vasc Access 9(4), 231-5 (2008)). There are currently no clear algorithms for the management of prosthetic vascular graft infections. The basic principles, however, involve systemic antibiotics, debridement of infected tissue, partial or complete graft excision, and secondary revascularization (Bunt, T. J. Cardiovasc Surg. 9(3), 225-33 (2001)). However, many patients presenting with vascular graft infections have significant comorbidities and are often critically ill, making surgical management even more difficult and in certain cases ill-advised. Despite best management, mortality and morbidity rates remain high with conservative estimates for both over 20% (O'Connor, et al., S. J Vasc Surg. 44(1), 38-45 (2006); Perera, G. B., et al., Vasc Endovascular Surg. 40(1), 1-10 (2006)). Reinfection rates are also significant after initial treatment, highlighting the inadequacy of current treatment modalities at eradicating the infecting organism. As the number of procedures involving vascular grafts continues to rise with an aging population and increasing prevalence of atherosclerosis and diabetes, new strategies are sorely needed.

P. aeruginosa infections are notoriously difficult to manage due to low antibiotic permeability of the outer membrane and mechanisms of antibiotic resistance that allow cross resistance to multiple classes and types of antibiotics. Clinically significant levels of antibiotic resistance are mostly caused by interplay between the efficient outer membrane (OM) permeability barrier, ubiquitous periplasmic β-lactamases, and multi-drug resistance (MDR) efflux pumps. These pumps have broad substrate specificity and may act synergistically with the permeability barrier to result in significant intrinsic resistance to many antimicrobials. These pumps expel the antimicrobial from the cell into the surrounding space, and the antimicrobials then have to pass through the OM permeability barrier to regain entry to the cell. Thus, the MDR pumps can effect significant resistance even when their transporter activity is quite low, as long as the OM functions as an effective barrier.

Synergy between efflux and the permeability barrier is necessary for effective drug resistance. Efflux pumps are transport proteins that are found in both Gram-positive and -negative bacteria, as well as in eukaryotic organisms. Pumps may be specific for one substrate or may transport a range of structurally dissimilar compounds (including antibiotics of multiple classes); such pumps can be associated with multi-drug resistance (MDR). Efflux pumps can also impact iron uptake, bile tolerance, quorum sensing, and other host colonization factors.

In the bacteria domain, there are five major families of efflux transporter: MF (major facilitator), MATE (multidrug and toxic efflux), RND (resistance-nodulation-division), SMR (small multidrug resistance) and ABC (ATP binding cassette). All these systems utilize the proton motive force as an energy source, apart from the ABC family, which utilizes ATP hydrolysis to drive the export of substrates. Transporters that efflux multiple substrates, including antibiotics, did not arise in response to the stresses of the antibiotic era. All bacterial genomes studied contain several different efflux pumps; this indicates their ancestral origins. It has been estimated that ˜5-10% of all bacterial genes are involved in transport and a large proportion of these encode efflux pumps.

The interaction between selection from antibiotics and phages, along with its role in driving bacterial evolution, remain unclear, in part because these interactions depend on the environment, specific phage species, and the bacterial hosts involved in these interactions. Potential evolutionary interactions between drug resistance and phage resistance mechanisms in bacteria have been previously identified, including both positive and negative interactions that are highly genotype-dependent. For example, Pseudomonas aeruginosa bacteria that evolve resistance to phage 14/1 simultaneously become more resistant to antibiotics, whereas P. aeruginosa that evolve resistance to phage tivp become less resistant to antibiotics.

Escherichia coli, bacteria that evolve resistance to phage TLS also lose antibiotic resistance. Such interactions demonstrate that multiple selection pressures sometimes cause bacteria to evolve mutations with trade-up potential (the ability to increase fitness on two traits simultaneously), whereby phages contribute to the problems of increased antibiotic resistance and virulence; in other cases, the mutations have trade-off potential, whereby phages reduce the problem of antibiotic resistance. In both cases of trade-offs and trade-ups, the mutation selected for one function has a pleiotropic effect on another function. This type of pleiotropy, sometimes called molecular gene pleiotropy occurs when a single gene affects multiple traits. For example, the evolution of bacterial resistance to phage can occur via mutations that also decrease cellular resource acquisition (a form of antagonistic pleiotropy), and mutations that improve bacterial growth on one carbon source may improve fitness during growth on another (a form of synergistic pleiotropy). In these ways, pleiotropy can have significant effects on evolution within bacterial populations, shifting phenotypes that are not directly selected.

Pleiotropy in the evolution of phage resistance and antibiotic resistance can result in either decreased (antagonistic pleiotropy) or increased (synergistic pleiotropy) sensitivity to antibiotics. Bacteria-phage interactions can be highly dependent on cell membrane proteins and other surface structures, such as outer membrane proteins and lipopolysaccharides, and in some cases, those structures also contribute to antibiotic resistance. In particular, multi-drug efflux pumps are protein complexes spanning the inner and outer membranes of some bacteria, such as the homologous TolC-AcrAB system in E. coli and OprM-MexAB system in P. aeruginosa. These efflux systems confer resistance to multiple antibiotics, acting as generalized transporters for multiple antibiotic classes as well as detergents, dyes, and bile acids. The outer membrane protein (OMP) components (TolC or OprM) are membrane-spanning beta barrels, with peptide loops that extend outside of the cell. The extracellular loops of OMPs are frequently exploited by phages as the specific binding sites for initiating phage infection. When phages use these OMPs as receptors, bacteria face selection for reduced or modified OMPs. Phages that use OMPs involved in antibiotic resistance, like the previously-characterized TolC-targeting phage TLS, might impose selection on bacterial populations to evolve phage resistance while pleiotropically losing antibiotic resistance.

A need exists in the art to develop alternative methods for the management of antibiotic efflux of MDR in bacteria, like P. aeruginosa and E. coli. The present invention addresses this need.

### SUMMARY OF THE INVENTION

As described herein, the present invention relates to compositions comprising bacteriophage OMKO1, LPS-5, TIVP-H6, LPS-TLTL, TIVP-27, SFA1-1, SF60B, SFNHSI, SF37B, PG-I1, and U136B, and methods of use thereof.

In one aspect, the invention provides a method of increasing antibiotic sensitivity and/or decreasing virulence in a pathogenic bacteria. The method comprises contacting the bacteria with a lytic bacteriophage selected from the group consisting of OMKO1, LPS-5, TIVP-H6, LPS-TLTL, TIVP-27, SFA1-1, SF60B, SFNHSI, SF37B, PG-I1, and U136B.

In certain embodiments, the bacteria are contacted with bacteriophage at a multiplicity of infection of bacteriophage to bacteria in the range of about 0.05 to about 50.

In certain embodiments, the bacteriophage binds at least one molecule selected from the group consisting of an O-antigen, efflux pump, Type IV pilus, LPS (core), OmpA, OmpC, TolC, and peptidoglycan in the bacteria. In certain embodiments, the bacteriophage binds TolC and LPS.

In certain embodiments, the bacteriophage binds a protein of a Mex efflux pump. In certain embodiments, the Mex efflux pump is a surface exposed protein. In certain embodiments, the Mex protein is selected from the group consisting of OprM, MexA, MexB, MexX, and MexY.

In certain embodiments, the method further comprises contacting the pathogenic bacteria with an antibiotic.

In certain embodiments, the pathogenic bacteria is drug resistant. In certain embodiments, the pathogenic bacteria is a multi-drug resistant (MDR) bacteria.

In certain embodiments, the bacteriophage comprises a nucleotide sequence at least 80%, 85%, 90%, 95%, 96%, 97%, 98%, 99%, or 100% identical to any one of SEQ ID NOs: 1-13.

In certain embodiments, the bacteriophage is administered to a subject in need thereof.

In another aspect, the invention provides a pharmaceutical composition comprising one or more lytic bacteriophages selected from the group consisting of OMKO1, LPS-5, TIVP-H6, LPS-TLTL, TIVP-27, SFA1-1, SF60B, SFNHSI, SF37B, PG-I1, and U136B.

In certain embodiments, the bacteriophage binds at least one molecule selected from the group consisting of an O-antigen, Type IV pilus, LPS (core), OmpA, OmpC, TolC, and peptidoglycan on multi-drug resistant (MDR) bacteria.

In certain embodiments, the one or more bacteriophages comprise a nucleotide sequence at least 80%, 85%, 90%, 95%, 96%, 97%, 98%, 99%, or 100% identical to any one of SEQ ID NOs: 1-13.

In certain embodiments, the pharmaceutical composition further comprises an antibiotic.

In another aspect, the invention provides a method of treating a bacterial infection in a subject in need thereof. The method comprises administering any of the pharmaceutical compositions disclosed herein to the subject with the bacterial infection.

In certain embodiments, the pharmaceutical composition is administered directly to a site of the bacterial infection.

In certain embodiments, the method further comprises administering an antibiotic to the subject. In certain embodiments, the antibiotic is administered before or after or co-administered with the pharmaceutical composition.

In certain embodiments, the bacterial infection is drug resistant. In certain embodiments, the bacterial infection is multi-drug resistant.

In certain embodiments, the pathogenic bacteria is associated with a biofilm. In certain embodiments, the pathogenic bacteria is in a biofilm.

In certain embodiments, the pathogenic bacteria is Pseudomonas aeruginosa, a Shigella species, Staphylococcus aureus, or Escherichia coli.

In another aspect, the invention provides a method of disrupting a pathogenic bacteria associated with a biofilm. The method comprises contacting the bacteria with a lytic bacteriophage selected from the group consisting of OMKO1, LPS-5, TIVP-H6, LPS-TLTL, TIVP-27, SFA1-1, SF60B, SFNHSI, SF37B, PG-I1, and U136B.

In another aspect, the invention provides a method of preventing formation of a biofilm on a surface. The method comprises contacting the surface with a lytic bacteriophage selected from the group consisting of OMKO1, LPS-5, TIVP-H6, LPS-TLTL, TIVP-27, SFA1-1, SF60B, SFNHSI, SF37B, PG-I1, and U136B.

In certain embodiments, the bacteriophage binds a molecule selected from the group consisting of an O-antigen, efflux pump, Type IV pilus, LPS (core), OmpA, OmpC, and TolC in the bacteria and the bacteria either genetically resists bacteriophage infection or becomes infected and lysed by the bacteriophage, wherein the genetically resistant bacteria have impaired efflux pumps and increased sensitivity to one or more antibiotics.

In certain embodiments, the method further comprises contacting the bacteria or surface with the one or more antibiotics.

In another aspect, the invention provides a composition comprising an LPS-5 bacteriophage targeting Pseudomonas aeruginosa, the bacteriophage having a genome comprising a nucleic acid sequence comprising SEQ ID NO: 1.

In another aspect, the invention provides a composition comprising an OMKO1 bacteriophage targeting Pseudomonas aeruginosa, the bacteriophage having a genome comprising a nucleic acid sequence comprising SEQ ID NO: 2.

In another aspect, the invention provides a composition comprising a TIVP-H6 bacteriophage targeting Pseudomonas aeruginosa, the bacteriophage having a genome comprising a nucleic acid sequence comprising SEQ ID NO: 3.

In another aspect, the invention provides a composition comprising an LPS-TLTL bacteriophage targeting Pseudomonas aeruginosa, the bacteriophage having a genome comprising a nucleic acid sequence comprising SEQ ID NO: 4.

In another aspect, the invention provides a composition comprising an TIVP-27 bacteriophage targeting Pseudomonas aeruginosa, the bacteriophage having a genome comprising a nucleic acid sequence comprising SEQ ID NO: 5.

In another aspect, the invention provides a composition comprising an SFA1-1 bacteriophage targeting a Shigella spp. bacteria, the bacteriophage having a genome comprising a nucleic acid sequence comprising SEQ ID NO: 6.

In another aspect, the invention provides a composition comprising an SF60B bacteriophage targeting a Shigella spp. bacteria, the bacteriophage having a genome comprising a nucleic acid sequence comprising SEQ ID NO: 7.

In another aspect, the invention provides a composition comprising an SFNHSI bacteriophage targeting a Shigella spp. bacteria, the bacteriophage having a genome comprising a nucleic acid sequence comprising SEQ ID NO: 8.

In another aspect, the invention provides a composition comprising an SF37B bacteriophage targeting a Shigella spp. bacteria, the bacteriophage having a genome comprising a nucleic acid sequence comprising SEQ ID NO: 9.

In another aspect, the invention provides a composition comprising a PG-I1 bacteriophage targeting Staphylococcus aureus, the bacteriophage having a genome comprising at least one nucleic acid sequence selected from the group consisting of SEQ ID NOs: 10-12.

In another aspect, the invention provides a composition comprising a U136B bacteriophage targeting E. coli, the bacteriophage having a genome comprising a nucleic acid sequence comprising SEQ ID NO: 13.

In another aspect, the invention provides a method of treating a bacterial infection in a subject in need thereof. The method comprises administering to the subject a therapeutically effective amount of any of the compositions disclosed herein.

In certain embodiments, the bacterial infection is drug-resistant or multi-drug resistant.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1D illustrate the finding that phage U136B relies on TolC. FIG. 1A shows an efficiency of plating receptor screen of phage U136B reveals TolC as the candidate outer membrane protein receptor. A phage that produces an equal number of plaques on a knockout as on wild-type bacteria has an EOP of 1.0 (dotted line). A “bd” at the lower, dashed line indicates that the efficiency of plating was below the limit of detection (˜10⁴). FIG. 1B shows genetic complementation with a plasmid containing tolC fully restores plaquing ability by phage U136B on a tolC knockout. Error bars=95% CIs. FIG. 1C is a series of bacterial growth curves showing that phage U136B lyses wild type bacteria in liquid culture (˜1.5 h) but has no effect on the tolC knockout in liquid culture. FIG. 1D shows single-step growth curves that confirm phage U136B cannot grow on a tolC knockout in liquid culture.

FIGS. 2A-2C illustrate the finding that phage U136B relies on lipopolysaccharide. FIG. 2A shows results from an efficiency of plating screen of phage U136B on LPS synthesis gene knockouts revealing genes important to phage replication: rfaC, rfaD, rfaE, and rfaP. FIG. 2B shows genetic complementation with plasmids containing respective rfa genes fully restores plaquing ability by phage U136B on the knockouts. Error bars=95% CIs. FIG. 2C is a schematic of genes involved in LPS synthesis, showing that the genes required by U136B affect the deep region of core polysaccharide (figure adapted from Chang et al (2010) Journal of Experimental Microbiology and Immunology 14:101-107, Yethon et al (2000) Journal of Bacteriology 182(19):5620-5623).

FIGS. 3A-3I illustrate the trade-offs between phage resistance and antibiotic resistance. FIGS. 3A-3B show MICs for phage-resistant isolates from the fluctuation experiment. FIGS. 3D-3E show MICs for phage-resistant isolates evolved in +phage treatment communities. FIGS. 3G-3H show MICs for phage-sensitive isolates evolved in control −phage populations. FIGS. 3C, 3F, and 3I show a phage-mediated trade-off between colistin resistance and tetracycline resistance is evident in the fluctuation experiment (FIG. 3C), but this result is alleviated after evolution (FIG. 3F) and doesn't appear in the control treatment (FIG. 3I). In FIGS. 3C, 3F, and 3I, a jitter of ±7.5% has been added to the data points for visualization but regression lines are based on the original, non-jittered data.

FIGS. 4A-4B illustrate the evolution of antibiotic sensitivity in bacterial populations while under selection for phage resistance. FIG. 4A shows total bacterial population densities, and FIG. 4B shows tetracycline-resistant bacterial densities, in the presence and absence of phage.

FIG. 5 is a table illustrating phage U136B has a limited host range within E. coli, plaquing on strains with and without the LPS O-antigen. EOP=efficiency of plating (mean of three replicates). NA=not applicable.

FIG. 6 is a table illustrating the phage resistant mutations isolated from the fluctuation experiment. ^{a}A “Δ” indicates deletion of indicated base pairs or gene. A * indicates a point mutation resulting in a stop codon. Values in parentheses indicate either single substitutions or the location of either a stop codon or IS-element insertion within coding sequences. Underlined bases indicate single substitutions. ^{b} Concentrations tested were 0.125, 0.25, 0.5, 1.0, 2.0, and 4.0 μg/mL, so discrete value listed may be greater than the actual MIC but not more than the next highest value tested. Values are the mode of 4-5 replicates tested for each mutant. ^{c}All rfa gene names are synonymous with the corresponding waa gene names used in the BW25113 annotation. Mutations are reported in the rfa form for readability (e.g., waaP is equivalent to rfaP). ^{d}Concentrations tested were 12.5, 25, 50, 100, 150, 200, 250, 300, 350, and 400 ng/mL, so discrete value listed may be greater than the actual MIC but not more than the next highest value tested. Values are the mode of 4-7 replicates tested for each mutant. ^{e}The 48-gene deletion included LPS synthesis gene gmhA (also called lpcA), which codes for a phosphoheptose isomerase involved in LPS synthesis. A full list of the 48 genes and their annotations is included in FIG. 15. Gene ydeM putatively encodes a YdeN-specific sulfatase-maturating enzyme.

FIG. 7 is a table illustrating phage resistance mutations arising during experimental communities with and without phage. Control MIC values for BW25113 and tolC mutant are from the same dataset reported in FIG. 6. ^{a}A “A” indicates deletion of indicated base pairs or gene. A * indicates a point mutation resulting in a stop codon. Values in parentheses indicate either single substitutions or the location of either a stop codon or IS-element insertion within coding sequences. Underlined bases indicate single substitutions. ^{b} Concentrations tested were 0.125, 0.25, 0.5, 1.0, 2.0, and 4.0 μg/mL, so discrete value listed may be greater than the actual MIC but not more than the next highest value tested. Values are the mode of 3-4 replicates tested for each strain. ^{c}Concentrations tested were 12.5, 25, 50, 100, 150, 200, 250, 300, 350, and 400 ng/mL, so discrete value listed may be greater than the actual MIC but not more than the next highest value tested. Values are the mode of 2 replicates tested for each mutant. ^{d} narZ: nitrate reductase 2 (NRZ), alpha subunit; ryiB: novel sRNA, function unknown; arsC: arsenate reductase; yhiS: putative uncharacterized protein; bglH is a cryptic carbohydrate-specific OMP gene; metE is a 5-methyltetrahydropteroyltriglutamate-homocysteine S-methyltransferase gene; lplT is a lysophospholipid transporter gene.

FIG. 8 is a transmission electron micrograph showing phage U136B has a siphophage morphology.

FIG. 9 shows Kirby-Bauer disk-diffusion assay measurements for colistin sensitivity have high variance but yield similar relative results to the microbroth dilution method. The control strains (DrfaD and DrfaH) and phage resistant mutants (RGB-036 to RGB-079) had increased sensitivity to colistin compared to ancestor. Phage resistant mutants RGB-040, 045, and 071 contain tolC mutations. Error bars show SEM of 11 to 19 independent measurements of each strain.

FIG. 10 illustrates the finding that phage-resistant mutants vary in fitness costs. Bacterial growth curves include wild type bacteria (BW25113), tolC phage-resistant mutants, and LPS-related mutants listed in FIG. 6. Each line shows the mean of three biological replicates.

FIGS. 11A-11B illustrate location of point mutations and small INDELS in the TolC amino acid sequence for strains from the fluctuation experiment (FIG. 11A) and evolution experiment (FIG. 11B). Insertion-sequence (IS) mediated mutations are not shown. (Figure adapted from Koronakis et al. (2000) Nature 405:914-919).

FIG. 12 is a table illustrating bacteria and phage used in the study.

FIG. 13 is a table illustrating sensitivities to colistin in rfa gene knockouts. Data summarized from Table S18 of the genome-wide screen reported by Liu et al. 2010 Antimicrob Agents Chemother 54(4):1393-1403.

FIG. 14 is a table illustrating phage extinction dynamics during the evolution experiment. Timing of phage extinctions was based on plating filtered phage samples as spot tests, top agar overlays, or both. At Day 10, Population +9 was not detectable in the serial dilution spot test, but it did have plaques appear in the filtered phage sample.

FIG. 15 is a table illustrating genes in the 48-gene deletion. The only gene readily related to LPS synthesis is smhA (row 6), a phosphoheptose isomerase involved in core polysaccharide synthesis.

FIG. 16 is a table illustrating mutations in experimentally evolved Tet^{R} isolates. ^{a}A “Δ” indicates deletion of indicated base pairs or gene. Values in parentheses indicate either single substitutions, or the location of a stop codon, INDEL, or IS-element insertion within coding sequences. Underlined bases indicate single substitutions. ^{b} All rfa gene names are synonymous with the corresponding waa gene names used in the BW25113 annotation. Mutations are reported in the rfa form for readability (e.g., waaP is equivalent to rfaP). ^{c}gatR: pseudogene, repressor for gat operon, interrupted by IS3, split galactitol utilization operon repressor, fragment 2, split galactitol utilization operon repressor, interrupted; ^{d}argV one of seven arginine tRNAs. ^{e}moaB: inactive molybdopterin adenylyltransferase ^{f}yaiX: pseudogene, interrupted by IS2A, acetyltransferase homolog, nonfunctional, interrupted by IS2, putative transferase; ^{g}metE: 5-methyltetrahydropteroyltriglutamate-homocysteine S-methyltransferase; ^{h}lpIT: lysophospholipid transporter.

FIG. 17 is a table illustrating statistical analysis for phage-mediated trade-offs between colistin resistance and tetracycline resistance. The trade-off is only observed among fluctuation assay mutants (“Fluctuation” model) and not after 10 days of evolution with phage (“Evolution+Phage” model) or in the control populations (“Evolution-Phage” model).

FIG. 18 illustrates phage that target Shigella flexneri bacteria. Phages SF37B and SF60B cannot kill bacteria where OmpA and/or OmpC are missing. A plasmid with OmpA or OmpC can ‘rescue’ killing but empty plasmid cannot.

FIGS. 19A-19D illustrate S. flexneri phage SFA1-1. FIG. 19A shows data from the “cross-streak” method showing the phage cannot kill (disrupt bacteria growth) when bacteria lack OmpA. In contrast, the phage effectively disrupts wild-type (M90T) growth. FIG. 19B shows phage SFA1-1 cannot kill bacteria where OmpA is missing; a plasmid with OmpA can ‘rescue’ killing but empty plasmid cannot. FIG. 19C shows phage SFA1-1 prevents wild type (M90T) from growing (but phage-resistant mutants appear and grow around hour 14). In contrast, M90T, M90T without OmpA, and OmpA knockout plus phage all grow roughly equally. FIG. 19D shows results from absorption (attachment) assays showing phage binding (disappearance from media) occurs only on wt and ompA plasmid-rescue strains.

FIGS. 20A-20B illustrate details of phage SFA1-1 genetics and molecular biology. SFA1-1 was isolated from waste water in Cuatro Cinenegas, Mexico. It belongs to the Myoviridae family (likely Tevenvirinae), is a dsDNA virus, 105,517 bp contig assembled, 139 CDS, and GC content 35.89% (S. flexneri GC content 50.9%).

FIG. 21 illustrates phage that target Pseudomonas aeruginosa. Related data for LPS5 phage: PA01 bacteria have a frequency of resistance to LPS5 equal to ˜ 7.2×10-4 (i.e., mutants observed at roughly 1 in 7,200 cells).

FIGS. 22A-22B illustrate P. aeruginosa phage TIVP-H6. FIG. 22A shows burst assay data that estimate phage TIVP-H6 growth traits on PA14 bacteria. Burst size—14.8 particles released per infected cell; Latent/Eclipse time—40 minutes. FIG. 22B illustrates InSeq Screen to test for putative binding of TIVP-H6 on PA14 bacteria. Every point is a normalized counts-per-million read for a single gene. Statistically significant genes included those involved in Type IV pilus biogenesis; strongly suggests Type-IV pilus is binding target.

FIG. 23 illustrates P. aeruginosa phage TIVP-H6 causes trade-off where bacteria evolve resistance that causes reduced pyocyanin production. Shown is bacteria pyocyanin relative to untreated control. Ciprofloxacin, Tetracycline, Nalidixic acid, Carbenicillin, and Dead (heat inactivated) phage TIVP-H6 do not reducte production. But phage TIVP-H6 reduces pyocyanin production on par with Eythromycin, and in combination with Cip and Ery. When P. aeruginosa bacteria are exposed to treatments along x-axis, the supernatant of the culture is tested for pyocyanin level. Exposure to TIVP-H6 yielded lowest level, such that bacteria die from phage attack and remaining mutants are phage resistant and produce very low pyocyanin levels, comparable to Ery which does not kill bacteria in this assay but causes reduced pyocyanin production. Thus, H6 does both, and works well in presence of Ery and other antibiotics that do not affect pyocyanin or even cause increase.

FIG. 24 illustrates P. aeruginosa phage LPS-5 causes trade-off where bacteria evolve resistance that causes reduced elastase production. Classic congo-elastase assays confirmed that elastase production in culture supernatant was below the limit of detection when bacteria were grown for 48 hours. Data compared wildtype PA01 and 3 spontaneous phage-resistant mutants of PA01.

### DETAILED DESCRIPTION OF THE INVENTION

As more alternative therapies are considered to combat the rise of antibiotic resistant biofilm-associated infections, the use of bacteriophages, as described herein, presents a novel strategy to manage these difficult to manage infections.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of 20% or within 10%, 9%, 8%, 7%, 6%, 5%, 4%, 3%, 2%, 1%, 0.5%, 0.1%, 0.05%, or 0.01% of the specified value, as such variations are appropriate to perform the disclosed methods. Unless otherwise clear from context, all numerical values provided herein are modified by the term about.

As used herein, the terms “antibacterial activity” and “antimicrobial activity” with reference to a bacteriophage, isolated bacteriophage protein (or variant, derivative or fragment thereof), or bacteriophage product, are used interchangeably to refer to the ability to kill and/or inhibit the growth or reproduction of a microorganism, in particular, the bacteria of the species or strain that the bacteriophage infects. In certain embodiments, antibacterial or antimicrobial activity is assessed by culturing bacteria: gram-positive bacteria (e.g., S. aureus), gram-negative bacteria (e.g., K. pneumoniae, A. baumannii, E. coli, and P. aeruginosa) or bacteria not classified as either gram-positive or gram-negative, according to standard techniques (e.g., in liquid culture, on agar plates), contacting the culture with a bacteriophage or bacteriophage product and monitoring cell growth after the contact. For example, in a liquid culture, the bacteria may be grown to an optical density (“OD”) representative of a mid-point in exponential growth of the culture; the culture is exposed to one or more concentrations of one or more bacteriophage or bacteriophage product, and the OD is monitored relative to a control culture. Decreased OD relative to a control culture is representative of a bacteriophage or bacteriophage product exhibiting antibacterial activity (e.g., exhibits lytic killing activity). Similarly, bacterial colonies can be allowed to form on an agar plate, the plate exposed to a bacteriophage or bacteriophage product, and subsequent growth of the colonies evaluated related to control plates. Decreased size of colonies, or decreased total numbers of colonies, indicate a bacteriophage product.

By “attenuated” is meant the bacterium has a decreased virulence with respect to a wild-type bacterium. In particular, a bacterium has an attenuated virulence of about 10, 20, 30, 40, 50, 60, 70, 80% or more decrease in virulence as compared to a wild-type bacterium.

As used herein the terms “bacteriophage” or “phage” refer to a virus that infects bacteria. A “lytic bacteriophage” is an actively infecting bacteriophage, i.e. one that is in the lytic (virulent) cycle (as opposed to the (dormant) lysogenic cycle). In the lytic cycle, the phage takes over the host cell's machinery for its own replication, ultimately killing (lysing) the host cell to produce its own progeny. The term “bacteriophage products” refers to polynucleotides, polypeptides, or fragments, variants, or derivatives thereof, isolated from a bacteriophage of the invention, which polynucleotide, polypeptide, or fragment, variant, or derivative thereof, exhibits a biological function or activity associated with the bacteriophage from which it was isolated or derived (e.g., antimicrobial or antibacterial activity (e.g., lytic cell killing)).

By “effective amount” is meant the amount required to reduce or improve at least one symptom of a respiratory disorder, condition or disease relative to an untreated patient. The effective amount of airway epithelial cells used for therapeutic treatment of the respiratory disorder, condition or disease varies depending upon the manner of the specific disorder, condition or disease, extent of the disorder, condition or disease, and administration of the cells, as well as the age, body weight, and general health of the subject.

The term “efflux pump” refers to an active, protein transporter localized in the cell membrane that exports substrate(s). In bacteria, five classes of efflux pumps exist: MF (major facilitator), MATE (multidrug and toxic efflux), RND (resistance-nodulation-division), SMR (small multidrug resistance) and ABC (ATP binding cassette).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, adenoviruses, and adeno-associated viruses) that incorporate the recombinant polynucleotide.

A “vector” is a composition of matter that comprises a gene and that may be used to deliver the gene to the interior of a cell. Vector refers to any plasmid containing the gene that is capable of moving foreign sequences into the genomes of a target organism or cell.

As used herein, the term “fragment” as applied to a nucleic acid, is less than the whole.

By “host” or “host cell” is meant a cell, such as a mammalian cell, that harbors a pathogen, such as a bacterium. The pathogen can infect the host cell.

By “immune response” is meant the actions taken by a host to defend itself from pathogens or abnormalities. The immune response includes innate (natural) immune responses and adaptive (acquired) immune responses. Innate responses are antigen non-specific. Adaptive immune responses are antigen specific. An immune response in an organism provides protection to the organism against bacterial infections when compared with an otherwise identical subject to which the composition or cells were not administered or to the human prior to such administration.

By “infection” is meant a colonization of the host. Infection of a host can occur by entry through a membrane of the host, such as a phage passing through the cell membrane of a bacterium.

The term “bacterial infection” means the invasion of the host organism, animal or plant, by pathogenic bacteria. This includes the excessive growth of bacteria which are normally present in or on the body of the organism, but more generally, a bacterial infection is any situation in which the presence of a bacterial population(s) is damaging to a host organism. Thus, for example, an organism suffers from a bacterial infection when excessive numbers of a bacterial population are present in or on the organism's body, or when the effects of the presence of a bacterial population(s) is damaging to the cells, tissue, or organs of the organism.

By “infectious disease” is meant a disease or condition in a subject caused by a pathogen that is capable of being transmitted or communicated to a non-infected subject. Non-limiting examples of infectious diseases include bacterial infections, viral infections, fungal infections, and the like.

The term “isolated” refers to a material or an organism, such as bacteria, that is free to varying degrees from components or other organisms that normally accompany it as found in its native state. Isolated denotes a degree of separation from an original source or surroundings. An isolated bacterium is sufficiently free of other bacteria such that any contaminants do not materially affect growth, pathogencity, infection, etc. or cause other adverse consequences. That is, bacteria are isolated if they are substantially free of bacteria or materials. Purity and homogeneity are typically determined using analytical techniques, for example, single cell culturing. The term “purified” can denote that a cell gives rise to essentially one population.

By “multi-drug resistant,” “multi-drug resistance” or “MDR” is meant antimicrobial resistance to the effects of antibiotics or other antimicrobial drugs.

By “non-pathogenic” is meant an inability to cause disease.

By “pathogen” is meant an infectious agent, such as bacteria, capable of causing infection, producing toxins, and/or causing disease in a host.

By “disrupt” is meant to kill bacteria and/or to inhibit, slow, stop, or prevent bacterial replication and/or growth.

By “associated with a biofilm” is meant that the pathogen is present in and/or on a biofilm or forms a biofilm.

A “portion” of a polynucleotide means at least about twenty sequential nucleotide residues of the polynucleotide. It is understood that a portion of a polynucleotide may include every nucleotide residue of the polynucleotide.

“Proliferation” is used herein to refer to the reproduction or multiplication of similar forms, especially of bacteria. That is, proliferation encompasses production of a greater number of bacteria, and can be measured by, among other things, simply counting the numbers of bacteria, measuring incorporation of ³H-thymidine into the bacteria, and the like.

As used herein, “sample” or “biological sample” refers to anything, which may contain the cells of interest (e.g., cancer or tumor cells thereof) for which the screening method or treatment is desired. The sample may be a biological sample, such as a biological fluid or a biological tissue. In one embodiment, a biological sample is a tissue sample including pulmonary arterial endothelial cells. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

The term “strain” means bacteria or bacteriophage having a particular genetic content. The genetic content includes genomic content as well as recombinant vectors. Thus, for example, two otherwise identical bacterial cells would represent different strains if each contained a vector, e.g., a plasmid, with different phage open reading frame inserts.

A “subject” as used herein, may be a human or non-human organism. Non-human organisms include, but are not limited to, livestock, pets, aquaculture organisms, cultivated plants and crops. Preferably, the subject is human.

As used herein, the terms “treat,” treating,” “treatment,” and the like refer to reducing or improving an infectious disease or condition and/or one or more symptoms associated therewith. It will be appreciated that, although not precluded, treating an infectious disease or condition and/or one or more symptoms associated therewith does not require that the disorder, condition, disease or symptoms associated therewith be completely ameliorated or eliminated.

In the context of treating a bacterial infection a “therapeutically effective amount” or “pharmaceutically effective amount” indicates an amount of a composition comprising bacteriophage which has a therapeutic effect. This generally refers to the lysis of bacterial cells or, to some extent, of the acquisition of resistance (genetic evolution) of bacterial cells to bacteriophage infection.

By “virulence” is meant a degree of pathogenicity of a given pathogen or the ability of an organism to cause disease in another organism. Virulence refers to an ability to invade a host organism, cause disease, evade an immune response, and produce toxins.

By “bacterial virulence” is meant a degree of pathogenicity of bacteria. Bacterial virulence includes causing infection or disease in a host, producing agents that cause or enhance disease in a host, producing agents that cause or enhance disease spread to another host, and causing infection or disease in another host.

By “virulent” or “pathogenic” is meant a capability of a bacterium to cause a severe disease.

By “wildtype” is meant a non-mutated version of a gene, allele, genotype, polypeptide, or phenotype, or a fragment of any of these. It may occur in nature or produced recombinantly.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DESCRIPTION

“Phage therapy”, the application of lytic bacteriophages (or “phages”; viruses of bacteria) for the bio-control of bacteria, is one method for treating multi-drug-resistant (MDR) bacterial infections: the use of lytic (virulent) bacteriophages (bacteria-specific viruses) as self-amplifying ‘drugs’ that specifically target and kill bacteria. Lytic phages bind to one or more specific proteins on the surfaces of particular bacterial hosts, an intimacy that led to development of phage therapy as a biocontrol strategy which predated use of broad-spectrum chemical antibiotics. Due to the recent precipitous rise in antibiotic resistance, phage therapy has seen revitalized interest among Western physicians, buoyed by successful clinical trials demonstrating safety and efficacy.

However, one limitation of phage therapy is the abundant evidence that bacteria readily evolve resistance to phage infection. While multiple mechanisms of phage resistance exist, phage attachment to a receptor binding-site exerts selection pressure for bacteria to alter or down-regulate expression of the receptor, thereby escaping phage infection. Given the certainty of evolved phage-resistance, modern approaches to phage therapy must acknowledge and capitalize on this inevitability. Genetic trade-offs are often observed in biology, where organisms evolve one trait that improves fitness (a relative advantage in reproduction or survival), while simultaneously suffering reduced performance in another trait.

Described herein is an evolutionary-based strategy that forces a genetic trade-off: utilize phages that drive MDR bacterial pathogens to evolve increased phage resistance thereby increasing sensitivity to chemical antibiotics. Thus, this approach to phage therapy should be doubly effective; success is achieved when phage lyse the target bacterium, and success is also achieved when bacteria evolve phage resistance because they suffer increased sensitivity to antibiotics.

### Antibiotic Resistance

Many strains of bacteria have become antibiotic resistant, and some have become resistant to multiple antibiotics and chemotherapeutic agents, the phenomenon of multi-drug resistance. Some strains have become resistant to practically all of the commonly available agents. For example, methicillin-resistant Staphylococcus aureus (MRSA) is resistant to not only methicillin (which was developed to fight against penicillinase-producing S. aureus) but also aminoglycosides, macrolides, tetracycline, chloramphenicol, and lincosamides. Such strains are also resistant to disinfectants, and MRSA can act as a major source of hospital-acquired infections. An old antibiotic, vancomycin, was resurrected for treatment of MRSA infections. However, transferable resistance to vancomycin is now quite common in Enterococcus and found its way finally to MRSA.

The emergence of “pan-resistant” gram-negative strains, notably those belonging to P. aeruginosa and A. baumanii, occurred more recently, after most major pharmaceutical companies stopped the development of new antibacterial agents. Hence, there are almost no agents that could be used against these strains, in which an outer membrane barrier of low permeability and an array of efficient efflux pumps are combined with multitudes of specific resistance mechanisms.

Efflux pumps belonging to the resistance-nodulation-division (RND) family of transporters are the major multi-drug efflux (Mex) mechanism in both E. coli and P. aeruginosa. The pumps in this family consist of three components that function via active transport to move numerous molecules, including antibiotics, out of the cell: an antiporter that functions as a transporter (e.g., MexB, Mex D, MexF, MexY), an outer membrane protein that forms a surface-exposed channel (e.g., OprC, OprB, OprG, OprD, OprI, OprH, OprP, OprO, OprM, OprJ, OprN), and a periplasmic membrane fusion protein that links the two proteins (e.g., MexA, MexC, MexE, MexH, MexX). This system is the major efflux pump associated with intrinsic resistance among 17 possible RND efflux pumps in P. aeruginosa. P. aeruginosa is more resistant than E. coli due to a highly impermeable OM and the presence of multiple efflux systems. Inactivation of the Mex efflux pump renders P. aeruginosa more vulnerable to antibiotics than the average E. coli strain.

### Compositions

In one aspect, the invention includes a composition comprising a lytic bacteriophage selected from the group consisting of LPS-5, OMKO1, TIVP-H6, LPS-TLTL, TIVP-27, SFA1-1, SF60B, SFNHSI, SF37B, PG-I1, and U136B.

In certain embodiments, the bacteriophage binds a molecule of an efflux pump on pathogenic bacteria, drug resistant bacteria, multi-drug resistant (MDR) bacteria, and/or pan-drug resistant (PDR) bacteria.

In one embodiment, the bacteriophage binds a protein, such as a surface exposed protein, of a Mex efflux pump. In another embodiment, the Mex protein is selected from the group consisting of OprM, MexA, MexB, MexX, and MexY. In various embodiments the lytic bacteriophage is selected from the group consisting of OMKO1, LPS-5, TIVP-H6, LPS-TLTL, TIVP-27, SFA1-1, SF60B, SFNHSI, SF37B and PG-I1. In various embodiments the bacteriophage binds a molecule selected from the group consisting of O-antigen, efflux pump, Type IV pilus, LPS (core), OmpA, OmpC and peptidoglycan.

In certain embodiments, the bacteriophage utilizes the antibiotic efflux pump protein TolC and LPS.

In yet another embodiment, the composition further comprises an antibiotic. The antibiotic includes any commonly available agent, such as an antibiotic selected from, but not limited to, amoxicillin, erythromycin, penicillin, ciprofloxacin, azithromycin, ceftolozane/taxobactam, ceftazidime/acibactiam, tetracycline, imipenem/carbapenem, and any combination thereof.

The present invention also includes a pharmaceutical composition comprising the bacteriophage described herein. Pharmaceutical compositions comprise the bacteriophage in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); Magnetic Resonance and Computerized Tomography contrast agents; and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In another aspect, the invention includes a composition or a pharmaceutical composition comprising the bacteriophage described herein, wherein the bacteriophage disrupts the multi-drug resistant (MDR) bacteria, Pseudomonas aeruginosa.

Details regarding various bacteriophage of the invention are presented in Table 1, below.

TABLE 1

| Phage strain | Sequence | Bacteria | Putative Target |
|---|---|---|---|
| LPS-5 | SEQ ID NO: 1 | Pseudomonas | O-antigen (LPS) |
| | | aeruginosa | |
| OMKO1 | SEQ ID NO: 2 | Pseudomonas | Efflux pump and |
| | | aeruginosa | Type IV pilus |
| TIVP-H6 | SEQ ID NO: 3 | Pseudomonas | Type IV pilus |
| | | aeruginosa | |
| LPS-TLTL | SEQ ID NO: 4 | Pseudomonas | LPS (core) |
| | | aeruginosa | |
| TIVP-27 | SEQ ID NO: 5 | Pseudomonas | |
| | | aeruginosa | |
| SFA1-1 | SEQ ID NO: 6 | Shigella spp. | OmpA |
| SF60B | SEQ ID NO: 7 | Shigella spp. | OmpC |
| SFNHSI | SEQ ID NO: 8 | Shigella spp. | |
| SF37B | SEQ ID NO: 9 | Shigella spp. | OmpC |
| PG-I1 | SEQ ID NO: 10-12 | Staphylococcus | Peptidoglycan |
| | | aureus | |
| U136B | SEQ ID NO: 13 | E. coli | TolC and LPS |

In another aspect, the invention provides an LPS-5 bacteriophage targeting Pseudomonas aeruginosa. In certain embodiments, LPS-5 binds a molecule of O-antigen (of a lipopolysaccharide). In certain embodiments, the LPS-5 bacteriophage has a genome comprising a nucleic acid sequence comprising SEQ ID NO: 1.

LPS-5 (SEQ ID NO: 1):
CGGTCGGTGCTCCATGGTACTCGGTCGGTGCTCCAACTGGTAACGCCAGAGAGCCGAGCG
ATGAAGAGTATCGGATGATCCTGAAAGCAAAGATCATCAAGAACAGAACAAACTCAACCC
CAGAGCAAGTTATCGAAGCTTATAAATTTGTATTCGGGGTTCCTGAAGTATTCCTAGAGG
AGTACGCTCCCGCTGCTGTCCGTATCGGCATCGGTAAGATTCTAACGAACGTAGAGCGTA
GTCTTCTATTCGACCTAGGTGGTGCAGGTGCATTGCTTCCTAAGACTATCGGGGTTAACT
ACACATACACTGAGTTCCAAGCTGGCCGGGTATTTGCTACAGAAGGCTTCCCCGGAGGAC
AAGGCGTTGGAGACCTAAACGATCCCACTGTTGGTGGAATTCTGACCAACCTAGTGACAT
AAGGAGTTATAAATGGCTGATATCGGTCAACTACCTATTGAAAATATTTGGTCCACAGGC
GGGGATATGGTAGCCCCGACCCCTGCGCAGCAGCAAGGCGGATGGGGTATTCAGTCGGTT
CCTCGCCAATGGTGGAACTGGAAGTGGAACCTTCACGATACTAACCTAGCGTATTTGCTA
CAGAAGGGCATCCCAGAGTGGACTAGCACTCAAGAGTATATTGCAAACAAATCGTTCTGC
ACAAGAAACGGTTTCGTCTACAAGGCTGTCCGTACCCATACAGGTAGCGACCCGGCCACC
GTTAGTTCTAACTGGGTCCGAGCTTTCGCAGACTTTACCACAGCTAGCTCTGCACTTGGA
AGCCTGACTCCGAGAGAAGGAGGGATTCCTTTCTTCATTAGCGCATCTGGTGCAGGTGTG
TTTGACTCTACGGCTTATGGACGTGGCATGCTGAACGTAGCCAACGCAGCGGGAGCTAGA
GACTACATTTCGGCTCAAGAGAGTTCTGTTGTCCTGTCTAACCTGTCTACTGTGACTAGG
GCTGCCAACGCGGTTCCGTACTTCAACACCGACACCTCGATGGCTACCTTCAACATTACA
GCGTTCGGAAGAGGCCTAGTCAACGCGGGTGATGCAACTAACGCAAGAACCTTCCTAGGC
CTAGCTAACTCTGCTACTATCACGGCTGATCCAGCCAACAGAGCACACACTCTGGTCTAT
AGAGACGCTGCTGGTAACTTCAACGCTGGTGTGATTACAGCCACTCTTTCTGGTAACGCG
ACTACAGCCAGCAAGCTCAGGGCTCCAGTTACGATTAACGGCGTAGCGTTCGACGGAAGC
CAGAACATTGTTCTGCCGGGCCTAGATACAAGCTACGCCGGGGTTGTAGCGAGGCTCCAT
ATTAATGGAGCTAACATGACCGGAACAGATAAGACTACCCAGTTGGCTCTTAGAAATAAA
TCTGATACAGATTGGATTAGCTTGGTGGCTCCTGATGACAGCTCGTTGGTATTTCAATTT
AGAGGGCCAGAAGCGGCTAACGCCACTCTGCGGGTTAACTCTAATGTTGTGTTCCACGCG
GGAAACCAGTATGGACTAGGAGCTACTCAAACGCAAGCCAGAGAGCGGCTCTTGGTTGAC
AGACTGGCACAGAACACGTCTCAAACTGTCCTGTACAACGCAGATAAGAGCAGGTACTTC
TGGTTGCAAGATAACGGTGTTTCCGGTATCTGGGACGCAACGACTAACACCCCTCTATGG
CGTTTTTCTAGACTAGGCCAGCTCGACTACGGAACAGTCCCTGTTGCACAAATTACAGGA
TTGGCCAACTCCGCGACAATTCCGGCATCCGCCTCCAATCTGACTAATCAGATTGTTGTG
AGGAGCGGAACAGGAGATATCGCAGTACAAGGCGTTACAGCGTTTGGCGCTGTTCTGGGA
ACAGACTATATTAGAGTAAGAGCCAGCACTGGTAACGCTCACCTAGTATTCCAGAGGAAT
GATGGAGCAGAGGTAGGGCTTATTTGGGGAATCCCGTCTGCCAACTCTCTGAACTTCAGG
ACCGCAGGCGGTGCGACAGGCATGTCTCTGGCTGGTCAGGACTTGACTGTAGTTGGCAGG
GTGAATGCTGCGACACTCAATTCGTCTGGCAACGTCAACTCTGGCAACAGCGTGATTGCT
GCCGGGAACGTGCAGGCTGCTGGCCGTGTGTACTGCGGCGATGCTTTTATGAGCAGCAAC
GGCAACCTTACTGGCGGAGCTTTCGGCTCTTATGGGACTCTTACCAACTGGGTAGACTCT
GTATACGCCAGAAAAACTGACGTAATGACTGATGCCCAAATCTTGGCTGCCACTTCTAGA
GGTAGTGCTCAGGCTGTGGGAACATATGTCTTTGCGATTGCAGCTAGCGGAAGTGGGGAT
GTTGGGACTGTAGTAGCTGGTACGTCTCTAAGGTCTTCGTCTCCCGTCAACTACCACGGA
ACTACGCTATCCGGTAGCTACAGGTGCATGGGTAGGTTCGGAGCCAGAGGTTCTAACCAG
ATTACCTTGTTCCAGAAGGTTGCAGATTAACAAAATAGGCCCGTAAGGGCCTTTATAACT
GGAGGTGATTTTAATGGAATTCAGAAACGTTGTAAAAATTAAAGCGGGGTTCGAGTGCGA
AATAAACCATCCAGAATTGGGATGGATTCCTTACGGCGCTGTAGAGGGATGCCCTACGTC
TCACTCCATGTACGTGAAGATCGCCGCAGCGATTGAAGATGGCTCTATGGAAGTGGTAGA
AAAAGAGGACAAATATGATGACTTTGAAGCCAGATTGTGGAGAGACAACGAGCTAACCCG
GGCTGACCGTCAAGTAGAGATTGCTATCGACGATGAAGACGAAGTAAGAGAGAAGGCTTG
GCGTAAGTATCGTTCTGCACTACGTAAATGGCCAGAGCACAAGAACTTCCCATCCGAAAA
GAGCAAACCTAAAGCTCCTAAAGAATAAGGCGAGGTAATATATGCCAAACATTATGAAGC
CTACGGGTATTAACGCTATCTGGTCTGAAAACGGCCAGAAGGTTGACCCGGGTGCTGTAA
AAGTTGGGCTTGGATGGGTGACAGAACTACCTCCCTACCAAACTGCCAACTTCATTGAAT
ACAAGCAAGACTTGTTCAACGCGCACGTTAACCAGCACGGTATTCCCGAATGGGATAGCG
TCACAGAGTATCAGGGTAACCTGAGCTACACTCAAGGCGCTAACGGGATCATCTATAAGT
GCCTTAGAACCCATTCCGACAAGATTCCAACTGACCCTCTGAACATTACAGCCGGATATT
GGCGAGTGGCGTTTGAGGATGCGGGAGAGGCAGCTAAGGTTCAAGCCAACCTTGATCGGC
ATGTAACAAACTACAACACCCTCTCTGGGATTGGCAACGTAGTTATTGCAAGACAAAACC
TAAGTGTGTATAGTAAAGCTGAAGGCGACGCCCGGTACGCTATGAAGCACGGGAACGGCT
CTAATGTTTTCAGCGTCGCCACTGCCACACAGCCAACTCACGCGATCCCGCTGAGCCAGC
TATCGACTCTCGTTCCTCCTGCTACCGAGACTGTAGCCGGGGTTATGGCAGTTGCTACGA
CTATCGAGACAGAAGCTGGAGCCAATGATACCAAGGCTGTGAGTCCGCTCAAGGCCGCAC
AGGTGTATCTGAAGAAGAAAGACAACCTTTCCGGTCTGTCTAACGTCACTGCCGCAAGAG
CTAATCTAGGACTGTCGGATACAGCGACTATGCCTTCTTCTACATTCCTGAAGGCAGGTA
GCAATCTGGCAGATGTGCCTAACAAGGCGCTGGCAAGATCGAATCTTGGAATTACAAGTA
GTGCTACCCAGCCGGAAACCTATTTCTTGAGGAGCGCTCAGAACCTAGCAGATGTACCTA
ACAAGGCACAAGCTAGAGTCAACCTAGGCCTCACTGGGATGGCTACAACAGACCCTGCGG
CGGTTATGATGAAGGCAGACAACCTTGCTGGATTGGCTAACACTGCTACCGCTCGGTCCA
ACCTAGGGCTAGGCACAGCTTCTACTAGAAATACAGGAGACTTCCTGTCGTCTGGGGCTA
ACCTGTCCGACCTCACGAACGTCCAAGCAGCTAGAAACAACCTAGGGTTGAAGGGAGCGG
CTACGCTCGATGTATGGGGTCTTCCTGCGAATACAACTGCTATGGACTTCCAGTCCAGTC
AGTCGGATATTTCCAGAGGATGGGCTCGACTACCAAACGGCCTTCTGCTACAATGGGGAA
CTGGTCCCGGTCTGTCTGATGACACAAGGACGAATATCCAACTCCCTGTTCCAGCACGAA
TCTTGAACGTTCAAGTGACCGTAATGGGGACGTTTAACAACTCCATCGGCCCGGGCGCTT
TCATGACGGATATGTGGAGCAACACCGGGTTTAGAGTTAGCTGCAACTGGGGCAACTGGT
CTTACCCGTTCAACTGGTTTGCTATCACTTCCACCCTGTAATTCAGGAGTATCAAAAATG
GCTCTAACTGAGCAAGACTTCCAATCGGCTGCCGATGATCTCGGAGTCGATGTTGCCAGT
GTAAAGGCCGTCACTAAAGTAGAGAGTCGTGGGAGCGGCTTTCTACTTTCTGGCGTCCCT
AAGATTCTATTCGAAAGGCACTGGATGTTCAAGCTTCTCAAAAGGAAGCTAGGTCATGAC
CCTGAAATAAACGACGTTTGCAACCCTAAAGCTGGAGGATACCTCGGCGGACAAGCGGAG
CACGAACGTCTAGATAAAGCAGTCAAAATGGATAGAGACTGCGCACTTCAAAGTGCCTCT
TGGGGCCTATTCCAGATTATGGGATTCCATTGGGAGGCACTAGGTTATGCGAGTGTTCAG
GCATTTGTCAATGCCCAGTACGCCAGCGAAGGATCGCAACTAAACACTTTTGTACGCTTC
ATCAAGATCAATCCTGCAATCCACAAAGCTCTGAAATCCAAGAACTGGGCGGAATTTGCC
CGGAGGTATAACGGGCCTGATTACAAGAAAAATAATTACGATGTTAAGCTAGCAGAAGCC
TACAAATCCTTCAAGTAAATCTAGGAAGGTTCCTAATGAAGAAGCTATTGAAAAGCTGGA
AGTTCTGGGCTGCTGTAGGCTCTGCAACTTTTATGGCTCTGGAGGCCATGGTTCATCTTT
GGGAGCCTATCCTTCCTCCCGGCGTATTCGCCGCAGTGGCCACACTGGTAGCTGTCGGTG
TAAGAATTTCGGCAGTGTTTGTAACTGCTGGTAAAGTGGAGGAAGCGATTTCGGAAACCA
TAGAAGATGGCTCAATAGAAAATGATACTAACTAAACTCAAAAACTACGCCCTCGTAGTA
GCTGCCGCAATGGCAGCAGGGTTTGGATATCTGTCGTTCTCTTTGTACGGCGACTTAGCT
GTTGCGAAGAATGATATCCAATCTCTTCAAGCTGCGTCTCGACAGATGGAAGAGCAGTTC
CAGAAAGAGCGAGCAGGGTGTGAGATCACCACTGGGAGACTAGAGGCACATATAGCCCAA
ATGAAGAGCCGACAGGAGCTTCTACAAGGCCACCTGTGTGAGCTTGAGTCTCTACCCAAC
CCAACCCCAAGGGAGACCCAAGAAAATGCCACAGAGACGATCTCTGAGCCTTCTACTGGG
GATAGTCTGGACCCTGCTCTTACAAGCTTGCTCGACAGAGCTTTCTGTTCCGCAGTACCA
AACGATCCTTATTGTGCCCCCGGCAAGCCTGACCGAGGAGGTTAAGGTATACCGAGTGGA
GCAAGATACAGTTAAGGGGCTCGCTAGAGCTTACATTCTAAATACTAACAGTGCTGCCAT
GTGCAATAGTCAACTACAAACAATAAAGCTATGGGCAGAAGAGCAGAGGGCTCTATATGG
AAAGTAAAGACGGCAAAGAAGGCCTAATCAAGGTAGCTACACAGGGAGTTATCTCTCTGC
TAATTGCTATCCTAATGGCTTGGTCTGGATTCCTAAGTTCCAGTCTTTCTAAGCAGGAAG
AACGTATCTTTGCGATGCAAAGAGATATGGTATCCGAGGCAAAACTTAACGCACTAGAGT
CGAGATTCAATAGCCGACTTGAAAGTGAAATTAGTAACGTAAGAAGAGAACAACAAGAAA
CCAACCGGTATTTGATTTTGATTATGAATGCCGTCAAGGGTCGGGATACAGAAACTCAAT
AACTATAACCCGTTGACCCACACTGGGAGACAATATAATGACAAGGAAACAAGACTGGCC
GCTAGTTTCAGCAGTCGGGGCTTTGTTTATCTCCCTCCTTGTCTGTATGGCTATTCTGGC
TTTCACAGCGATGACTCCCGAGGCTCGGTTTGACTCTGTACGCCAGACCTACGAGATTCG
AATTAGCGAATCTGAGCGGCGGTATGAACTCAAACTGAACAGACTACAGGATCAGATAAA
CTCGGCCTCGTACACACAGGATCGGCAATCCCGTTTGCAATATGAAGAACTCGACTCGCT
TAAAAGGAAGATTAGCGAACTTGAGCGGAGAATAAAAGAAAAACAAGAGTAATTAAGCCC
CGGAAGCGAAAGCTTACCGGGGCTATTTGTTACTGCTAGGTTACTTCCCACACTTCGTCA
AACAGCTTGTCAATTTTCGAGATAATCTTGTTTACCTTTTTCTTACGACGCTCATTATCT
TCTGGATCGGAATGGATAAGCTCGATGTCGGCAGCGTTACCCCAGTCCAGCTCCCCGTAA
TGGAACAGCACCTTGACGTTCAGGCAGGTCGCTGATCTGTCAACGCCTGCGACAACCCCG
GTGGAGCGCGAGTTGTGTTGCTCGAATGTACTTCCCGATGGCCGGTTGTACAAGTCGCTT
ACCTCTACAACTTGCCCCTTCTTAACGTCTTCCAGTTTCATTCTATGCCTCCTCAATCTA
GTTTCATTAGCGTATTGAACTGGTGCCTCAAAGACTTGAAAGCCTGTGGGCCATCGTCCT
CCGCTCCTTCATGGATATGATGAGCGATCAAGTGTATCAGTCCTCGGTCTCCTAGTAACA
CAGAGCTAGGAATCTCTTTTATCCAAGCCACAAAGCAACAGAAGTGCAGCACCTCCGGCA
TGGAAGCCTTGAAGCTGGAATCATCCAGCTTCGATAGGCGTTCATACGTGGCTTTGAGGT
CGGGATAGCTTAACTGATATGTGGCATCATCGTCAATATGCTGATCCCCTCTAACTACAT
ACTGCAAGCTCATACTACACCTCTATATCGTTGCACCATTACACCTTCTTGCCGCCTTCT
GCAAGACGTGCCTCTCGCTTGTGGTCGGAACGGGTCATGTTGTACTCGATCTTCTCGATG
ATAGCCCCTGCAACATCCAAACCCCGTACATTCGCATAGTCCATGATGCGAATGATAGCG
TCGGCCAGTTCCACCTCTTCCATCTTCCGCTCTGGCAAGTGATCGTCCATCAAATCCTTA
CGGACGCCTTCAGCAGCTTCCGCAATTTCAGTGGTAACGAGCATCAGCATATGCAGAGCA
TCACGTTTAATCGGCTCGCCTGTGACCAGATCGGTGTTCCACTTGCGGTTAACAACCTCA
AGACCAGTCATCATTTTGTTAAAACCGCTAACAAACTCGAAGACGGACTCTCGTTCGAAT
AGTTCACCGAACGTTGGCTCTTGATGTTCGGCAATTGCAGTGTGGAAAGGCCACTCCATC
CAAGCGTCATAGCACTTTTTGATAAGCTCTTGTTCATCAATGTACGAGAAGACGCCCTGC
TCCTCAAGCTGACTAATAGCTTCAGCCCCCTCGACCCAGACGCACGTACCAATAAGCCTT
GGATCGATTCTCAGAGAAGTCTTGTAGACTTCTTTCCACGACCTCCATCCTACCCTACCA
TTGCCAAAATACACAAGGAGTGGCACATAGCCTTCTCGCTTTTCCTGCTCCGGCGTTTCT
ATAACTACAACAAACGCCGGAACTTTCTCCCCATCTGGACGTTCAGTTAGTACGAAGTCT
CCCAAGTTGATATTCTCCCGACTAGCTGGGCGAAGAGATTCCTCGATATGGTCTTTAACT
TTACTCATTAACTTCTTCCTCCTCACAGAACATTTCAAAATAGCAATCACAGCATACCAA
CATGCCGCCGTCATTTACAATTTCTTCGACCGGGAGAATGCTTCCACAGCATTCACACCC
AGCCCGTTCGACAGCATCTGCACAGGCCCAATAGTCCATCTTCACCCTCCTTCACTCCTC
CTTCAGAAGGTACTCGTTGCTAATAACCTTGAAACTAAAGTCTCGGCTCATGCTTTTGAA
CACCAAACCTTCCCGGTACTTACCGTTCAACGCAGACTCTCCGCTAGCCATTTCAATTAC
ATCTTGCATGTCTTTTGGAACGCCTACGTCCCACATGTACACCGGCACATGTTTAACATC
CTCTTCGCCGTCATCAGCTAGCGCGTTCAGGATTCCCAGAGTGGCCAGTCGCTCTTCTGG
AAGATAGTAACGACTCTCCGAAATGTTGTAGATATCGTACACATAGTATTCCGGTTTGTC
AACTCCCTCGAAGTTGCCTTGAATATTCGGTGCAACCAGTTCACCCTGAAGGGCGATATT
TAGACCAAACTCCCGGAGACGTTCACCAAGATTAAGCTCTTCTGCCATCGCAGTGAAGTG
ATTGGAGTCTTTCTTAATCACACGCTTCCAGCCACGACCCTTAAAGAAGTTCTTGACAGC
CGTGAAGAACCCGATCTTCTCGTCTTCTAGACGGAAACCGACATTTCGAGACGCCACGCC
TACTTCTCCGTCCTTGTGCCAGATAGTGAGAGACGAACCGTCCAATTTGAATGTCACTTC
AAACTTCTCGTCCTTCTGATGAGCCTGATCGTAAGCCATCTGAATATTCTGTACGCGAGC
CTGATCCGTCTTCGGGATGAAGTTGGGGAACCCTAGCTTGGAAGTGCCAGTCCGGCTGCC
CTTGTTGCCTCCGTTGATAGCTACCTGCTCGGAAGGCTTTACATACTTCAGGATGCCCAT
TCGATCGGTAACATCTTCGTCTACTTTCGGCTCTGGACTGCCCGCTGCATGAACAGGTAC
GATCAGTCCTTGGCTAAGCTCTTTGCGAAGCTTCATGGTCTTGATTCGAGAATACTCTTT
TCCACCTTGTTCGACCATATTACGGCCACGCAGGAATTCAAAGATAGGGTTGTCTACAGG
GACCATGGAGTCCACTTCAATGTAGATTGCCAGATCGCCAGCCTTATACTCCCCGCGTTT
GATTACGCACTGCCAACCTCCAACAAGCGCCAGATCAAGGCTGTCAGCGTTAGGATGGGT
CTTCACATCGTCGATACGCACCACTCGGGCTAGTACGCGGTCTTGGGAATTGCTCATATT
TCCTCCTTTATTTTAAATTTACTAGATTAATTGTCAAACCAACCGAGATTAAGGGCGTGC
TCCAAGGCTGCGAGAAGATTCTCAGCGTCCATGCGGTCGCTAATTGACACGCGGCGCCAT
GTGATGCCCTCTTTGTTTCTAGCTACAATATAGGCCGAGGAACTATCTCTTCCCTTTATT
AACGAGCTTGCAAACTCAGTCTTTCCCCCGAACACTATCTCGCTAACTTTTTGGGACGGC
TCTCGAATATCAATCTTCATCGTGCTCTCCTTGTTAAAAATTGTTAGATTTGATAGATTA
CTTGTTAAACCAACCGAGGTCAATAGCTCGTTCTAAGGCTCGTAGCAGATTCTCAGCATC
TAGAAGATCACGGATCGGCATCCAACTCCCACTATTCCCCGGTAAGCCATCTGCGATGTA
CACAGGTACGTCAGTATACTCGCTCTTTTTAAGCCCGCAGGCACAGGCTACATTGTCTCC
GAAGACAATCTCGCTGACTTCCCTTGCCTCGTCTCGAATATCAATCTTCATTATGTTTTT
CTCCTGTTGTGTTGAAAGTCGTTGTATTCTGCATGGATTCATCTGCTGTGTCAATAGCCT
TGACAAGAAAATAATCCGTGGTAAAGTGTGAGGCCTGCTGGAGAGAAATGTCCACGTCAC
TCACCGGCCAGCAGTATAGCGTATGAAGAGGAAAGCTGCAAGCGCATCCTGAGTCGAATC
TGAGAACAGGGCAGCGACGACCCGAAAGGGGAAAGCTCAGAGCCTATAAGGAGCCCATGC
TCCCCGCTCACCGACTCATCTGCATATGCGTATATCTGCATGAGAGCGAGAGGCAGACCA
GAGGCATGTCTTAGGCGGAGAATCCGTATGGCAAGCCTTGGGACAGGTCTGCCTCCACTA
CCGATCTATCTGCATACAAGAGCTTGACAGTATCCTGTAAACCGTGTAGAGTAATCTCAA
ATCAACCGCAGAGGAGTGTATAAATGAAGCATGTTATTCACCGTGAAGACCTTATCGACC
TACTACGTGATCGTATGAAGCTTGAAGCTCTAGAAGCTGGCGGGGTGGACAACTGGGACT
GGTATGAGGCCTCGCTAATCGACTGCGGATACTTCGATGACGATGGCCGGGAGCTAGAGG
TCGAAGCTCTTGCAGATTGCTTCCCAACCGTAGGTGCGTGACCGTGGAGGAGTCGAAAGC
TACCTTCAAAGACCCTTACCCTAGGTTCTTCGACGCTTCTCGTAGGTATGGCGAAACCAA
CGACTGCATGGTGGTGAGCTTTGCAGTCGTATGGAACACCACCTATGAAAAGGCACACCA
GCACCTGAAAGTGAAGTGCAAGCGCGGTTACCGGCAAGGGGCTACTCAGAAGCGCTTCAG
CTACGCAGACACTTGGTGCCCTAAGACCCGGATGCTCTACCGGGAGGATGCTAAGGATAA
GACTCTTAGGCAGTTTGCTGAGGAAAACCCTGAAGGTCGGTTCTGGATCGCGGTAAAGGG
GCATGCTGTTGCAATCGTCGACGGAGTGTTGTACGATCACACTAATGACAAGAGACGTAC
CATCTGGCACTGCTGGCAAGTGTGGCCGGGGGAGGCACCTGAGAAAATCGAGAGGAAGCC
TGATCCAATCTCACCTCCTGTGAGAAAGGCCCCGGACTGGTATGTCTAAACACAAACATC
TGAAGTACAAAGAGGAGGATTTATGGAAATTATTTCGAGAGGGACGCTTCCTCAAGAGGC
TTTGTTTACTGCTAAGTGTCACCATTGCCTAAGCATTCTCCGAGCTAAGAGATCGGAACT
TGAGTATGTTTCGTTCTCATTCTGTCGCCCCGGGGAGACTGGGTATTATTTCGCAGAGTG
CCCTGTCTGTCACGAAGAGGTGTCTGCTTGGGTTGCGGCTCCTGTCATTCCTGTGGCACC
GGCCACCACGGAGTATATCACAAAGGAGCGTAAATAATATGGACCAGTTAGCAGGATTCA
TGGGCGAACCTTTCGTAATTGCGTTCGACTACGACGAGACGATCAGTCTCTACCCTCTTG
CCTTCCGAATCGTCATGGATACCTTCAGGGGCTTTGGAGGCGCTTTCAATGTGATCGTAG
TCACTGCACGCCACCCTGACAACAGCGGAGAGTTAGAGTGGCTACGGGACTACGGGTTCC
CGATCTATTTCACGGCACAGAAGGCAAAGCGACCATTCATGGAAAATCTTGGAATAAAAG
TCTCTGTATGGGTTGACGACAACCCGTTCAGCGTGGATAATGACCTTCCTCCGTGGACAG
TGGAGCAATACGATGCCTACATGGAGGCAAAGAAGCAACGAGCTATCAACAGAGGGAGTA
AAGCATGAGCGAGATTATGAAGGTTGAAGTGGGAGATCGGGTGGTCCTTAAGGCCTATTG
CGATACATACACTGGATGTGCAAAGCGATGGGCGCAGGCGCATGGACGTTTGGATGAACT
GCTGGCTACGGGATTCGTAGAAGATAGCTGGGTCCACACCGACGAACTGGCCGGAGGCTT
GAAAGGCACCGTTGTGATGACTGAACTGGGACTGAAGGAAGAGTGCTGTGCAGTACGTCT
AGACGCCTACACTGACCACAGGGCTGTTGTAGTTGTTGACGCTTCTGATCTTTACATCGC
ATGTAAGGCAGCAAAAGCTCCCGTAAGCACCCTAGACGAGGCCTTCGGTTGTGACCACGA
CTACCTTATGGAATCTCCGGCTAACGCTCAGCGACTGAATGAGTCCGTCGAGCAGCTACG
AACTGGCCAAGTCGTAGAAGTGGACGTGCCTGTGAGTGTAAATCAAGACGAAACCTCGCC
TATCGTAGTGTATGTAGTAGAGAAGATTTACGATAGGAAAGGTAACGTACACCTCATTCT
ACAGTATGAGGTAGCAGGGGCGCTAATCAGGACGGTGGTGGAACAGGGAGAACTCATTTA
CTTCTTGGATGAAATGCAAGGTATGTACTTGACCAAGATTCAACTGGAGGTGAACAATGG
AGTTGTATGACCAGTGGCGTAAGTATATGAGCGTTGTAATCTTAGAAGAAGAAGATAATG
AGCGTCAGTGCATGCGGCTTATTGAGGCAGTTGGCGAACTCGATCCCTATATCCAAGATT
TCCTAGATTCTTTCTCTATTAAAACTGAAGGAGGTGTGTAAGCATGAGTCAGAGTAAGCG
TGATGAGATTGTAGTTGAAGCACGTGGGCTAGAGAGCTACTTCATTTCCCACTGGAACGG
CTGGGATGTGATGGACACTGCTTGTCTCGGATTTTATATCAAACCTGAGTATGTTCTTGA
CCCGGATATTCGAGAGCTTGAGCCTAGCTTCTTGGAGCTTGATTTCGAGCGTTGCCGTTT
CCGATTCTGGGGAAATGAAGACGAAGGCGTGGACGGCAAACTCATGGAGGGCAAGTTGCA
GATTATGACCAACTCCTTCGAGTACGTCTTCTTCAAGGAGGTTTGAGTGGCTAGACAGAC
TTACGAGTCTGCATCAGATAGGGCGGTCGAGGAGGCCGTCCGTTCTGAGCTTGAAGGGCG
GTGGAAGTGTTCTCTTCATAAGCTACCTAGAGCCCACTATATGGATTGGGCAGCCTGCCG
GAATGGAAAGGTTGTTAGCTTTGTGGAGCTTAAGGCTCGGAATAATGAAATGCAGAAATA
TCCCGACTTCTTTGTGTCCCAGATGAAGTGGCTCCATGGGATCGAAATGCAAAGGGTGTT
TGACATTCCCGCATTCCTTGTGGTAAAGTGGACGGACTATACAGGATATGTCAAAATGAC
AGCCAATGGCGTCAACTACATGAGCATGGGTGGTAGGATTGACCGTGGAGACTGGCAAGA
CCAAGAAGTTATGGTCCATATCCCGCTGAAAGACTTCACACGTTTATAGGAGGACAAATG
AAACGACTCTGCTACCGGTGTATGCACTTCGGGGAGCCCACCATGCACCGCCATAAGACG
GGCATTTGGTATAAGGGTTGTAGTAAATGTGGATCAGGAGTATTCTATGGCTAAGAAGAA
CCCTGTGTTTAAGTTTCTGAACACAGTCAACAAACCCTCGGTAGAGAGGGATAAAACCAA
ATACAGGCGGAAGCCTAAGCATAAACAGAAGGAGCGATTAAATGAAGAAGGGTGACAAAG
TGAAGATCAATGATGACGCTGATACCATCTTCCGGGGCCGTGTTGCCACTATCATCGCAG
TAGACGAAGAGGGTTGCGTTATTGATGTGGGCAGCTACGAAGACGAAAGCGGAGAGCTTA
TCGAGTACCTTCACCTGTTCTTCGAGCGTTCTGAGTTGTCCAAGCAGGTGTAGGAGGGTA
TAATGAACGGACCCGAGTGCAGATGGTGCGGAGAGAAAACTGAAGCTGAGTTCGTAGACG
TTGGGCTTGGATGGACGCAGCAAGTCACTGGAGGCATGTGTCACCAGTGTGGAGGGTACG
AGAACGGTTACTACCAGAACGACGGAAGGCTCACAGAGGTCGAGTTTGCCACCTACTGGA
GTGGTCCGTTCGAAGATTATGCGGATTTCTCGCCATTCAATCATATGAGAGAAGGCGACT
CGCCATGAAGGCTACACTGAGAAACCACAATCTGCTATACATCTACTTCCACACGTTCTC
GGAAATGAATGAGTGGTTGACAGAGAACGCTGTAGATCAATGGACAGTCACGACTCTGGG
CTCCGGCTATCAGGTGGCTGTTCCTATGGACCGTGCAATGCGGGACCAATACGTCTCGGA
GAGGGGTTGACACGAACACAAGGTGATGCTAAACTCCGTGGTTGTCTGAAGTAAGGAGGT
TTTATGTCCAAAATGACCGTGGCCCAAATTGCGGCTATGCACGGTATCGATTTGTATTCT
GACCACAAGACCGCTTGTCCCCAATGCCGGAAGAAAGGCGCTGACAACAGCGGAGACAAC
CTAAAGGTCTATGGTACTGATCGTAATACCGGGGAACACAAAGGTGCCTATTGTTGGGCG
TGTGGGTACACCTTGCTCTCTGAAGAGCAGGAAAGAGACGCCCGGGGAGAAGACGAAGAG
GAGTGGAATTTCGTGGGAACTGAATTTAACGCAGAGATCAACGCTCAAATTAAAGAGCAA
TGCTCGTATGATCCGCGTGGTTATCGTGGCTTGTCTAAAGAGACTTGCCAGTATTTCGGA
GTGCTACATGGTTTCGACCCGGAGACTGGCGAGATTGCAACTCAGCTATATCCTACTACT
ACTAATTACGAACTCACCGGTTACAAACAGCGGATCATGCCGAAGAACTTCTCAGAGCCT
ATCGGCGAGACCGGGAAGGATTGCGAGCTTTTTGGTCAGTTCAGGTTCAAGAATAGCAAC
AGCAAGAAGGTGCTTATTGTAGGCGGTGAGGTTGACCAGCTATCAGCATTCCAGATGCTC
AAGGAGTACGATGACCGGCGAGCAGGCGGTAAGAGTGATTATGAACCGACTCCAGTGGTC
AGCCCTACTATCGGTGAGAGTGGGGCATGGAAGCAGCTACAGGCTCAGTATGAGTGGCTA
GACCGGTTCGAGCAAATCATTCTCTGTTTCGACAGCGATGACGCAGGACGAGAGGCAGCA
GACAAGGCAGCCGATAAGCTTCCGAAGGGCAAGGTGTTCATGATGGAAATGAACCGTAAA
GACCCTAACGAGTATTTGAAAGCTGGGCGTCAATCGGAGTTTGTCAGTGCATTCTATCGA
GCCCGTACATGGACCCCTAGCGGTATTGTAGGGTCTGATGCACTCGACGCTCGAATGTAT
GAAGCTGTGCAGGTTGAGAAGATTCCTCTGCCACCGTTCGCTAAGAAGCTCCAGAAGGCT
ATGGCAGGAGGCATCCCGTTGGGCCGGATTGTGAATATCGGCGCTGCATCGGGCATGGGT
AAATCTACTGTAGTGGATGAGTTCACTCTGTTTTGGATTTATAACTCGCCTCATATGCCG
GGTACTGTGACTCTGGAATCGGACTGCGGGGAGTACGCAATCAAGCTGCTGAGCCGTAAG
GTAGGTGAGAAGGCAGACTTGTACGAGACCGTTGAAGAGAAAGTGGCGTTCCTTGATCGG
CACCGTGACGCTCGGTACGACTTGTGGCACAAGGAAGACGGAAGCCCACGCTTCTATCTG
ATCGATGACCGAGACGGAAGCCTAGCAGCCCTCAAGGCTCAAATCGAACAACTGATTATT
CAGTTTGAGTGCCGTGTTATTATTCTGGACCCGCTTCAGGATATTCTGGACGGATTGAGC
AATGAAGAGCAATCGGTGTTCATGCGCTGGCAGAAGGGTATGACCAAGAGCCACCGTGTG
ACGTTCATCAACATCAACCACGTTCGTAAGAGTGGTGGCGGTCAGAAGGCTAACTCGGCA
GGTGCGGAACTGTACGAAGAAGATTTCCAAGGGTCTTCGTCCATCTTCAAATCTGGTGCA
GCTAACATTCTGATGTGGCGGAACAAGGACGCGGAGAACGAGTTCGAAAAGAACCTGATT
AACGTGAAGCTCTCTAAGTGCCGATGGACTGGCCGCACTGGAATGATGGGCAATTGGTAC
TACGACGTTGAGACTCATACAATCTGGGACTTGGACGACTATCTGGACCGATACCCTGAC
AAGCGCCGTGAATACGAGGCTTGGATGGCTCAGAAGGAAGAGGAAGGCCGGGACACGGAA
TACTAAGGTTGACAGAGGGGTGATGCTTATGTATCATCCCTTTCGTCGTTTACAGGAGGA
AAGGAAATGCAACAACGATTTGAAACAATCGAGCAATACCTCGAATGGATGGGAAATAAC
AATTTCACCAACATGACTTGGGATATCGAGGCCACAGGCCTGTTGGATGAAACGTCTATC
GATTACACTGCCAGCCCCTACAAGATTAAGCCTAGCTACAAACACCACTGCATTGTAGTG
GAGGAGCACGGTGGCAATATTCTGGCATTCTACGATGGGCCGACCTACGTCTTTGACGGG
CGAGAGTACACTGAGAGACTGCCGAAAGCGTCTTCCATGCTCCACACTGAAGAAGTTGTG
ACTCTCAAGGACTACACTCCGGTAGAGTATATTCACCTGCCGCTGAAAGAGTTCAAGAAC
TATGTGCTCAAGCGCAAGATCAAGCGCGTAGTGGCTCACAACATGATTAACTTCGACCTT
ATGTCGATGAAGCTTGTGGAAGATATGGACTTCACTATCGGCGTAGAGCACAGGGATGGC
GGTCTGACTACGTGGTCTGCTGACACTTGGATGGGCAAGCAAGTGGTATTCGACGATACC
CTCGTTATCTCCAAGACTTGTAACCCTGACCTGTTCGGCGGTCACAGTCTGGATAAGCTG
TCTCGTCGTGCAGGTGGTGACACCAAGATTGACTTCCGTAAGCATTTGGCACCGGATGTT
CGATTCCTCGACTTCGCAGCCGATATGCTCTACTACTGTATCTATGACGTAAAGGCGAAC
ACCGCTGTATGGCATTGGTTGCAGGACCACGAAGATTACCAACTGTCCGACCGTGACAAC
TACATCAAGAATTGGCTGTCCCCGATTCAGTTGGAGAAGAAGGTTGCAGAGATCATCACC
TATCAGGAGCACCGAGGGTTTGTTCTGAATGTTGATCTCTGTGAAAAAGCTGTGAAAGAC
TTGGACGCCAAGATGGAAGAGCGTCGGGCCCGGGTAGAGCCTGTGCTTCCTCCTCGACCA
GCTACCAAGAAATTCATGGCAGACTACACTCCACCTGTTCGACAGTTCAAGAAGAACGGG
GAGATCACCACTTACCTACAGAAGTTCATTGAAAAGATCGGCGCTCAATTGAAAGAGGGC
GATGATCGGACTATGATCTTTGAGGGTAAGGAGTACAAGCTGCCTCTCCCGGCAGACGTA
CCTCTGAAAACGGAGATGCCTGCTAAGATTGGAGATACAACCCACATCAAGCAGTGGCTT
ATGGGCGAGTTCGGTTGGATTCCCTCTGAGTGGAAAGAGACGGACCTCACTGTCGACCAG
AAGAAGATCAAGCTGCCGATGGAAAACATCGTTGCTAAGATCGATCGCTGGCTGGATGTA
ACGTATAGCACAGCGTACAAGGAAGAGCGGCTGGACGGGTTCGGGGATTACATGAAGATC
ACCCCTCGTAGCACCCGCGCGTATGTACGTCAGAGGTTGATTGATCGTGCAAGCAAGGGG
AGCTGTAAGGTTCTCACTAACCCTAGCTTCACAAAGGGCCAAGAGAAAGAAATGTGTCCA
GACCTCGAACGCATTGCAGAACAGTTTCCGTTCGCGAAGGATGTGATTGAGTACCTGACC
TTTAAGCACCGGCGAAGCGCAATCCTCGGTGGCGGCATGGACTGGGACGAAATGGAGGAT
GGAGAGGAGCCGGAAAAAGGTTACCTTGCAAGCATTCGTCAGGACGGCAGGATTCCCACG
CCTGCTGACACTTGCGGGGCTGCTACTAGCCGGATGAAGCACCGGAAGGTTGCTAACGTT
CCTCGGATCACCTCGTTGTACGGGGCTGAGCTTCGAGAGTTGTTTGGGGTTGGAGATGGG
TTCTTCCAAATTGGATACGACTTCGATTCCTTGGAAGCACGTAAAGAGTCGGCCTACTGC
GATCAGTACGATCCGACAAAGGAATATTGCAAATCTCTTATGATGGAGAAGCCATTTGAC
GTTCACACAATGATGGCTAAGCGTATTTCTGAGATCATTGGTCGCCAATTTAACCGATCT
CCTGCGAAAAACGTAAAATATGGGTAAGAGATCAAGCCCATGTAAAACTGTGTGAATTCA
GGGGAAGACTAGAACAGTCAATCCTGAGCCAAGCTAGAGTGATCTAGAAAGGTGCAACGA
CTATCGAAACCACGGCGGGAGCCGGAAGGGAGTAGAGTAGGGTCAAGTGACCCGAAGCGC
ACAGGGCGCGAGAGCGTCATGATATAGTCTGGTCTGCATGGGAACATGCAGCGGTAACCC
TTGAGCAAGGTTACGGGGGAGGATTAACGACCCTCCCTGAACACAACGGTACATACGGCG
CTCAAGCTGGTAAAGTGGCGAAAACCATTGGTGCCGACTTGCAAACTGGTCAACTGGTGT
TTGATGCTTTCTGGGATGCTGCATTCCCATTGAAAGCTCTGAAGGACGATCTGACCAAAC
AGTGGGAAGCCAACGGTAAGAAGTATATTGTTGGAGTGGACGGTCGCCGGGTTCCTACCC
GTTCGGCTCACGCTATCCTGAACAGCTTGTTCCAGAGTGGCGGTGTTATCTGTGCTAAAC
GTGCTATGGTGATTCATGACCAACTGCTGGAGAAAGAAGGTCTGAAGGTTGACTTTTTCA
AAGAAGACTGGAAGAACAAGCCGGAATTCTGCCAACAAATGATCGCGTACCACGACGAAG
CGCAGTTGGAAGTAACCGCCAAGTCGGTGAGCTTCAAGATGTTCCCTTGGTCTCTTGTAG
GCGGTAAGCCAGAAGGAGAAAAGGAACAGGAGGAGGCGGAGAAGAAGCTGAAAGCTCTAG
CTCAGGCTTACAAGGACGAACAATTGGCTAGCACTGGCAAGGTGTGGAGTGATATCGCAC
ACTACAGCAAAGGTGAAGGCGGGGTGTTTGTAGCTTACTGCCGCGCTGGAGAACTTGCCA
CGAAGGCCGTGAGAGCTGCGGGAGAGTTCTACTCTACTCCTCTGGTGCCTATCGAGCTGA
CCGCTGGCTACATCGTTGGTCGATCTTGGAAGGATTGCCACTAAAACTAGCATTAAGCGA
GGAAAGGGGCTTGACAGGCCCCTCCGCCTCCTATACAATGGCCACATATTCACAACATAC
AAGGAGAAACACAATGACTCTGGCTTTCATTATCTATCTGGTAATGACCGTATTCCCAGC
CTTGTCGTCGGCTGGTAGCTTTGCCCTGTTCGTCACCATCATCGCATGGGTTGCAGGAGG
GTTCGTTTACTTGGCATCCATTGGTGGCCGAGAGAATGCGGCAGTTAAAGCCACACTCAA
AAATCACCTACAGCCCTTGACATTTAAGCTGTTCCTCCCTATCATTGTAGTCGGCCATCT
GGTCCCTTCGTCTGAGACCGCATGGTGGATGGTTGGCGGGTACGCTGCACAGAGCCTCTA
TCAAGCAGACGCTACTCAGCAGGTTCTCAGCGAATCTACTGAACTGCTCGAAGCTCTGGT
TAAGCGGGCTAAGAAAGAAACTCTGAAGGCTGTTGAAGGCAGCAGCGAGTGATACGGTAA
TCACAGAATAGGAGGTATGGTATGAACACGGCTCGACTCTACGTCTACGGCAAATCGTCC
ATGGTAGGAATCACTGTTGACAACAAGGCCAATCGGGAGTACTATACCTCTAAAGGCTTC
GAGGTATCGGAGACACCGTTAGCCCACAAAAGCGTGGCTAACAAATACAAACAGAAAGGA
GGTAAATAATATGTCTGGTGTTATTCCCGGGACTATCGTCAAACTCACTCAAGTGGGATG
TGACGTCACCGTTGGCTGGCCAAAGGAAGCCCGATATGTAGGCGCATACGCAGTCGCAGT
AGATGGACGCGATATCGGTCTCCCGGAAGGGGAAATCCGGTTCTGCTCCCTCCATTGGTC
TATTGACCGAGACGTCAATAATGGACTTGGAGATTACCAGTCGATGCTCGCTGACAACGA
CCGCTTCGAAATTGTAGCGGTCCCGGGCAAGAAGTATTATGTTAAGGCTGTTGCCGCACG
CCCTGACTTGATTTCCCACTACTTGGTTACTTCGGAACCAGCCTTGTACGAAGGCTTCGA
GTCGTGCTGGTTGAAGAGCATGCACGAAGGGGAGAGCCCGTTCTGCATGACTTTTAAAGG
ACGCGTTGACATTGGCGGTGAAACCTCTGACGAGTTGCAGTTCCAGACCCTCTATCCCGA
GTTTGTGCTGGAGGAGTTCAAAGAAGAGGACGAGGCAGAGGTTAAGGTTGAAGCTCCGGT
TGACGAAGAGAAGAAAGAAAAGGTTGCAAAGCCTGCTGTAATGGGTTATGCTGTTCTCGA
TGCAGACGGGAGTCCTCTTAAATTCTGCCTCACTCGCGAAGAAGCGCGAGCCACCAAGCG
CTGGTGGGGTGGCAAGGCTGAGGGAGTCTCGATTGTAAAAGTAATCAAAGGAGAGGAGGT
GCGTTAACGCGCTTCTCCTCTTGAAGAATCCGGGAATGTCCGGTATAATCGTCAGTCAAG
TGGCTGACACACTCGCCCAGTCTAGGGTACTTAAATAGGAGAAATAAAATGGCTTTTAAA
CTGAACGTAACTACCAACACTCAATCCGGCAACGGCGGCATTCGCAAGGAAGTCGATTGG
GATGCTCTGAACAACCATGTCGTTGAAGCTGCCGGCACTGCCGCTAAGGCGCGTTCGATC
CCGGGTTATATCTCCGGTATCATCGATCTCGGCGAGCAAGAGCAGAACGATGCAGAGGTT
GTGTTCACTGGCACTGCCGAAGATGAAATCCAAATCATCAAGGAATATCCGAACACTTAC
TTCAAGGATGGACTGGACAAGCAGACTCGCAAGCCTGTACGTCTGAAGTGCTGGCCGCAG
CGCGATATGCAACAAATCGCGATCACTGTGGACTTCCCTCAGATCGAGGTAGACAAGAGC
CTGTACCTGACCGGTAACAGCAACAAGTCTCCGCTTCGTGTCCTGTACAACGGCGACTTT
ACTCTGCCGGGTCAGAAGACTAAGATTGTTGGCCGTCCGTTTACCATCAAGGAAGTGAAG
CACCCGAACGGCAAATGGGCGTTCGCTAAGAACTCCATGCTGCACAAACTGGCTGTAGCT
ACTGGCGTCATCAACGAAGACGAACTCTTCACCAAGGACCGCGTAGGCGAGCTTCTGGGT
AAAGTGGCTCAGTTCCAAGTTCGAGTGTGGATGAAGCCGGGCAAAAACGACATGAAATTC
CTCACCGAGGATATCAGTCTGGTCGGTATGGTGCCTGAAGGTGTTCCGGTCCCTGAACTG
GATGAGAAGTACATCTACGGCGTGAACCTGTATGGTGAGAACGACGAAGAGGCAGTTAAG
CAACTGCGTGCCAACGTCATCAACACCATCAAGCGAGCCAAGAACTACGAAGGTTCTGAT
ATCAAGGCGATGATCGAAGCTGTTGAAGCTGCACGCGGTGGTAACCGCTCCAGTGGCGAC
AGCGAAGAGCAAGCTGAAGATAAGCCGCAGACCCAAGCTGCACCTAAAGTGGAGCCGGTT
CAAACCAACGTAGATGATGACATTCCGTTCTAAAAGACGGTGGTAAGACAAGGGGCTTCG
GCCCCTTCTTTTCAAAAATTCAAAGGAGGCAACACATGCAAATCAATTTTGGTCCCATCT
TCCTTCTGGCTGCTATCGTTCTGTGTGTACTGAAATCTGCCGGAGTTCTCGCTATCTCTT
GGACTGTGGCACTCATGCCCATCTGGATTCCTCTTGTAATTGTATTGTCGATTCTCGTAC
TGTTTCTTGTAATTGTAGGTCTGATTTACGCATTCGGTGATCGTAGTAAACTGAACTTTA
GCTTTAAAAAGCACTTCCGGAATAAGTGAGGACTAATGATGAGTAGTTTGGTTATTGTTC
GAGTACCTGAGTACATTCTAAAAGGTCCCACAGTAAACAACCAGTTCAGCGGTGACCTGT
GGGAGCGAAATCTCTTCAAAGATTTGAAGAACGCTGCGGAGTGTATCATTAAAGGAGAGC
CGGCTTATGTTGCTCTTCCTAACAAGGTAAACCCTGACACTCTACAAACAGAGTGGGACA
TTCAGGTTGTACAGTTCTAACCCAAGGGGAGCTTCGGCTCCCTTTTTCATTGGAGATAAA
AATGAGTGAAAAGGAAGAGGATATCGTCCTCATCGACGGCGACCTTATCGCCTTTAAGTG
TGCAAGTGTCAACGAGACCCGCTCCATCATTGTCAGAAACAAATTGACAGGAGAAGAGGA
AACTTGGAAGAATCGGACGACTTGGCGGGAGAATAACAAAGACGAAGAGGGATTCAACGA
AGACAACTTCGAAATTGAGGACCATCAAGACCCCAAGCACGTGTCCTACGGTATCTCTGT
TGTTAAGACCATGATCGACCGAATCTGCCGCCAAGCTGGGTGCAAGCAGTTCAAGATTCT
CCTGTCCGGTCCTGACAACTTCCGCGATTCCATTCCCCTCCCGAAAGAGTACGAGATTAC
CAAGGGTAAAAAGACCTTCACTCGTGGCGGCAGGTACAAGGGTAAGCGTACAGGGCAGAT
TAAGCCTCTTCAGCTAGGTCAGCTAAGGCAGTATATGATCGAAGCGTATGACACCATCAT
CCATCCCGGAGAAGCGGACGACTTGATGGCCGAAATGATGTACAAGAACGGTGTAAGCTA
CTCCAGAGGGGAGACTACTCAGCGCGTGATCGGGGCTACCATCGATAAAGACGCAGACGG
CACTCTAGGATGGCTTTGTAACTACGAACGTGAGCCTGTTCAAGTCAAGTTCATTTCAGG
CCTAGGATCGCTCTACAGGGACTCTAAGGGCAAGGTGAGAGGCGAGGGACGTAAGTTCTT
CTACTTCCAGTTGCTGTTCGGCGACCCTGTTGACTGCTACAGACCTGCCGACCTGTGCGT
AGGGAAAGACTTTGGAGAAGTGGCGGCATACAACGTCATCAGTCCCTGCACAACGGACAA
GGAGTGCTGGCAAGCAATCTACGACACCTACAAATCGTGGTATCCTGAGCCTGTCACTTA
CACAGCATGGGACGACACAGAGCACACTAAAGATGCAATCGATATCATGCAAATGTATTG
TGACTGTGCTCACATGCAACGCTGGGCAGGAGACAGAGTGGACTGTAGGGCGGTGATGGC
TAAACTCGGGGTAGACTTAGGAGGTGCAGAATGAGCGTAAGGCCTCGCCCTTGGGAAGAC
TTCCCGGGCATCTGGAAGACTGAAGCTGCGTTCTTGAGTTGGGTCAGAGGAGGCATTCGC
CGCTATCTCTGGTCTAAGAACCCAGTCAAGCTTGAGTTTGCTAAATCCCGAAGGATTAGG
ATAACTAACCCCAGCGAAAAGAGCAAGCTCAGGAATCCCACTGTATGGGGCTATGTGTGT
GAGCAGTGTGGAAAGGAAACTCCTCAAGCTAACGTCGAGATTGACCACAAGACAGGGGAA
TTCTCCCTGAAGCGAGTGGAGGACATCCAATCGTTCGTGGAAGGTGTCGTGTTTGTCCGC
ATGGAGGATTTGGCAGTGCTGTGCAAACCGTGCCATGAGATCAAGACATACGCCGAGCGC
TACGGGCTGACCCTAGAGGAAGCAGATATCGTAAAGCAGGCTATTGCAATCCAGAAGCAA
AAGGGATATGATAAGGTCTTCTTGCAGGAAGTGGGAATCAAACCCGCATCCAACGCAGAG
CTTAGAAAAGAGCAGATCATCGCTCATCTTAAATCAAAGAAGGAGGGTATATGAGAGAGA
AGTTTCTTAAAATCATGCGCAAGCTGCCCATCAGGGTAAGGGCTTCCCTTGTTCTGGTTT
GGATTGCTCTGTTCTTCTGGCTCCCGGTTGTAGCAGCATTAATTGCCGGAGTTATAGACT
TCTTTTGTACTGCAAAGGAGCAGTTGGAAGAGGGGTACTCTGTTCTGTTCAATGCCACTC
AGGGCGCGATCTGCTGCATTAAAACGGGGAAGGAGCTGGAGTTATGACCAATCTAGCTAC
ATATGTTATCCGCCACAAAAAGACTAAAGAGCTTTGGACTACTCCTAAAGGGAAACGTCA
ATGGACCACGAAAGGAGGAGCGGCCTTAGCTTGGAGGAACGCATATGATAGTACTACACT
GGGAGCGTACTATGCTAAAAGAGATATGGAAAAGTGGCCCGACCTGAACATTCCTTTGAT
TGTAGAGACGGGCATCCATCGTCTGGCTAGCTGGGAAGCAGCATCTTTTAAAAATCAATC
TGCTTTTGAGATTGTTGATATTGCTGAATCCTTGAAGCACGAAAGCCAGTTGGTGCTCGA
CCTACTGAAAGATGTTATTGGGTTCGAAGTGCCAGATCACCTCGGAGACGGATACGTCCG
ACTCACCATTAAAACGTCCACATACGACGATATCATCAAACTACTGAAGGAGGCAAAATG
AGCATGACTGACCCCCGAGCGTATTTCATCAAACAGGAGCTGGAGGAAGCAACGAAGCTT
CTAGAAAGAGCCCTCAACCTTCTAGATAACGTCCATTGCTACGATACAGATGTATACGAG
GACATTGCAGCATTCCTCTACGGTCAAGAGTGCGAGGAGGAATAAGGTGCAGCGCGATAT
TAGTAAAACTATAGCAGAACTTCGGGACTTGATCGAAGCTGTCTCTATGCTCGACGAGGC
CTTGGTTATGATGCAGAAAAACGGACTATATGATACGGACTTGTACAAACGTATCGATGC
ATTTTTGTATTATCAACCGGGCGAGGGCGAGTGGGAGGAAGTAGAATGAGCAGCAAGAAA
CACTTTGTAATCTCTGATACACAATGCAAACCGGGTATCAGTCTGGATTACATGGAAGCC
ATCGGTAAATATATCGTGGCAAAACGTCCCGATGTTGTGATTCACATCGGGGACCATTTT
GACCTAGCTAGCCTGTCATCCTACGATAAGGGTAAGCGCTCCTCTGAGGGCCGTAGATAC
GCCGAAGACGTTCAAGTCGGACGTGAGGGTCTGGATCGGCTGTTTAAGCCTCTGTGGGAG
CTACAGGAGCGTCAACGGGCTAATCGGAAGAAGGTGTACCAACCTCGGTTGATTTTCACA
CTGGGCAACCACGAAGAGCGGGCTGATCGAGCAGCGAACGATAACCCTGAGTTGTATGGC
ACCGTGGGTAGCGATGAGCTAGGGATTCAGGACTACGGTTTTGAGGTTGTCCCGTTCCTC
AAGCCTATTGAAGTAGATGGCATCTACTACGTCCACTATCTTCAAAACCACTTCACTGGC
AAGCCTCTAGGTGGTACAGCGAACAACATGCTCAAGGCTGCTGGTCATTCGTTTGTCATG
GGCCACCGTCAGCTTCTGGACTGGGCAATCCATCCGACAATCGACGGTAAACAAAGGCTT
GGCATTGTGACTGGAGCGTGCTATCCTCACGACGAGGCTTACAAAGGCTATCAGGGCAAC
AATCACTTCCGGGGTTGTGTTATGATTCACGAAGTGAAGGATGGTTTTGGTCTGCCGATG
CCAGTGAGTCTAGACTTTATGATGGAGAAAGGAGGTTTCAAATGACAGTTTATAAGTTCT
GGTGTGAATGGGACATCGGTATCAATGAATACCTGTGGCGAGACTACTATCAGATGGAAG
AAGATGTAGCTAAAGCCTTGTCAGACTGCGGGATTGAGGATACTATTGAGGAACTAGAGG
GCGCAGGCCTGCTCGGTTTCGACTCTGTTAAAGTGATTGGTTAATAGGAGGGCATATGGC
TGAGTTTTTCCTTGGGGCGGCCCTTGCAGGCTTGTTCTTTATGTTTAGTATCACTGCGAG
CTTTACCGCCGAAGATATCGAGAAAGTGACTTCAACATGCGAGAATAACGAAGGCGTCAA
GTATTTCGGAGTTGACTTCTTCGGAGACGGTGCTGTACACTGCAACAGCGGCGCTATTTT
CAAAATCGACCGTAAATCTAAATAATCGAAAAGGAGATATAAAATGACTGATTTCAACAT
CACCCCTGAGCAAATGGAAGAAGCAATGGCAGGTATGAACCCGGAGCAGCGGGCTCTGGT
AGAGAAAGCCTTGACCGACCGTAAATCTCTGGAAGATTCCATCGTGACCGAGGACGGTGA
ACTGGACTATGGCATGCTTGCACGACACATCCGCACCAGCTTGGGTGATCTGCCTCTGGT
AATGGGTGGCCCTGACTTCCTGAAGCTGATGATCTTGGTTGCTGCGATGCCGGAGACCAA
AGACGATCTCCTGACTCTGCACATGAACCTGTCCTTCATTCTGGACTGCATTCGTGAATC
GATTAGCGACGAGGAGTAACAAGTGGCCGAAGAGATTGTAAAAGAAGACCTATCTGGCGA
CGATCTCATGGTTGACTCAGCAGGGCTAGATGCCCTGCTGGCCTTCCTAGAGAGTCAGTT
TAAAGGTCCGAAGGGTGTACTGAAGGGATATGTCACTATGAGAGTGATGTGCGCTAAGAT
CGAGAGTCTTGACGGGTTTCAAGAGTTGAAAGATATGGAGGTGAACTTTGAGTAACGATA
TCATCGACGTTCATGCTGGGCAACCGATTTCTCTGGTGCAACGGTCAAAGAATCCTATCA
ATATTCAATATTGGGACGCCGATACCATCCCTCACGGCACAGTCTTGGTTGTTACCGAGA
AAAGGGGCGATATCCAAAAGGGAGAGTTGGTCGTAAGGCTGGCGAGTGGTGCAGATGACG
AGCACTCTGTTAAGGTATGTAGATTCTCTGATTTCTTCCTCCTAAGGGAGAGAGGTTATA
ACCCAGCCAGTCTCGGCGACGTTGAACAAGCGTATGATGTTCTGTTCGAAGACCCTCCGG
CGACTACCTTTGTTAACTGGGATCGGGCTCAGCCTTTGACAAACAGTGAGCGCTACTACG
GAGACTTTCTGATTGGCGGCGACCCTAAAGGGATGCCACTATTCAACATGGCTGTTCTTA
AAAATGTTCTGTCGCCTTACACTGCCGACGATGAGGAACTTTCAGCTATGTCTGGGAATC
CCGAGACAGTAGAGCACAAAGAAGTTGACGAACTAAGTGAGAGGGACGAGGCTTTAGCCG
AGGTCGACAGACTCAATGAAGAGCTTGTTGCACTGGGTAAGAAGGTTGTGTTCGCCAACC
TAGAAGATTACCTAGGCAAAGAAAACTATGCCACATTGGAAGCTATGGCTAACCACAAGG
AGGCTGTAGGTAGCGGAGGCTCCTCTAGTTACTACACTGTCAAGGTGAACAACCCCACTA
CGAAGGCTAACATGCCGTATTTTGCAGAGTGCAACGATATCATCGAAGCGCTGGATATGC
GATTCGGTCAAGGCAACATGTTCAAAGCGTTGTGGCGGGCTGCGGCAGCACAGAACCTAG
GCAAGCTGAAGGCCGGTAACGATGTTATCCGGGACTACGAGAAAGTCATCTTCTTCGCTC
AGTTGGAGATCGATAAGATTCTCAGAGAAAGGGGTTGACATTCTCCCCGAGGCTTGCGAT
AATAGGCTCCAAGTTGAAACGAAGGAGGTGTGTAGATATGATACAGAAAGTAAAACAGAA
GCTCATGATGGTGCATCCTATCCTCGCTAGCGAGATCGTCTGGTGCCTCACCTTCGCAAT
GCTTCAGTAAAACAAGGGAGCTTCGGCTCCCTTTATTGTATCAAAGATTTAATAAGGAGA
GTTTATGCAAATTTCTGCAAAAGTGATTCAACACAGTTCCCGGATCGGCCTAGGTACGAA
GAATCACAGATTCATCACTACATTCGAACTGGAGTATCCTCGGTTTATTCACAGTGAACT
TATGACTCACCGACTTTTTAGTCGAAATGCTGCAAGTAGTCGTGCCATTCCCGTTGCCCA
TATGATCCGACAGGTGGAAGAGAATCCTGCGATGCCTGTTCACTGGGGAATGAATCAACC
GGGTATGCAAGCTGAGTTCGAACTGAGTGAGTGTTTGCAGCGTAGCTCTCAGTACCTGTG
GAAGAAGGCTGCAAAGAGTGCGGCTCGGATCGCTAGCGCACTAAATAAAATGGGACTCCA
CAAGCAGGTGGTTAACCGTCTCCTTGAGCCATTCCAGATGATGAAGACCGTTGTTACAGC
CACGGAACTAGATAACTTCTTCTACCTGCGTTGCCATAAAGACGCACAACCGGAGATCAA
GGTTCTCGCTGAGAAAATGTATGAGGCTCTTATTAAAGCGGGCCCGGCAGAGGCTCTATA
TGACGGAGAGTGGCATGTTCCTTATGTGACTCGGTTCCGCATAGGGGATGGTAAGCTGCG
TTACGCAGATTGTAACGGGCAAGAACTGAGCGTAGAGGATGCTATTAAGGTATCTGCTAG
CTGCTGTGCTCAGGTTAGCTATAGGAAGAACGACGAGACTCTGGAGAAAGCTCTTGTAAT
CTACAACCGATTGGTAGATACTAAGCCCGTTCACGCTAGCCCGTTCGAACACCAAGCAAC
ACCGATGCTCCGCATCTCGGACAATCAAACTGGATGGGAAGACGGTGTGACGCACTTGGA
CCAATATGGTGACCTCTGGTCTGGTAACTTCCGAGGTTTCATCCAACATCGTCAACTAAT
TAAAGGGCACGTATGTAATGAGTACTACGGCCAAGAAGAGGAAGGAGTTTAAAATGGCAG
AATATAACCTGAGTCTGGAAGACCTCATGCTGGTCGATGGCTTCAAAGAGGCATTCCAGA
GTAATAATGAGAAGGTAGTGAGGGAGCATCTGTGGACTAACGGCATGGATGTTAAAAACT
ACTCGTATGAAATGGTTTTCTGTCAACATCGCACACTGATTGGCCGGGTCGTAGAGGGTC
TACGATTCTCTGGCTTTGAGCGGACAGATAAAGAGTGGCTGAGCCTCGGTTGCGCCTCTC
TGGAAGCTCATATTGCAGCGTGTGATGATAGTAATCTACGGTTCACTCTTCGTAAGATGC
GACCAGAGGGATCGACAGAGGCAACGTTCCACAATTAATGCAGTAGGAGTAGATATGGAA
GTTACAAAGGCTCTCGCAGATGAGGCCGATATTTTCAATAAGATCACACAAATCAGGACT
CCAACGGAGTCCTATGCCCGGAAATATCCGGTCATTGTCGAACTAGCAAACCAGCAACTA
GAAGAGAAGTTGTGGTTCTCTAGCGAAATGAAGGTAGAGCTGGATAAGTTGAACCTGAAG
TTTAAGTTGGAACCTCATCAGCTTCACGCTGTAAAGACTGTCCTTCAACTGTTCCTCAAA
TACGAACTGATCGTAGGGGAGGAGTTTTGGATGGGTAAAGTAGTCCGAACGTTCCCTCGC
CCAGAGGTTAAACTAGCGGCTTCGATCCTGTGCATGATGGAATTGGCAGTCCACGCTGAA
TTCTACAACCAGATCAACGTAGTGCTGGGGATGGATACAGATGAGGACTATGTAGCTTAC
AAGGATGACCCCGAGCTTATGGGTCGAATGGATTGGCTTGAGTCTGTCCTAGGCGATGAA
GACGATGTTCTTTCGGTAATTATCTTCAGCCTAACAGAGACTGCCCTTCTGTTCAGTTCT
TTCGCTATCCTGAAGAGCTTCCAGTGCAACGGGTATAACGATATCCCTGTCATCGCACGC
GGGGCTAACCAGAGTGCTGTGGATGAAGACTTGCACGGTGTAGTTTCGGCTGAGATCATC
AACCAATACTACGCAGAGATTGGGAGACCATTGTCTGAAGATACTCGGCGTGTCGAGAAG
ATTCGACAAGCTATCGACCATGTATATGCTCATGAATGTCGTATTGTCGATATGGCGATC
CCGGGCGGAGAACTTAATGGGGAGTCGGCTGAAAACTATAAAGCGTTTGTTCGTTATCGT
TTGAATGTCTGGTGCCGAAGACTCGGCCTAGAAGATCACTTCGAAAACGATGACACTCCG
ATCAAGGATTGGTTCGAAATGAACACTTATGCCTACAAGATGATCGACTTCTTCACTCCG
GGTATGGGCATGGAATACGAACTAGGGTGGGATGAAGAGGAATTGGCAAAGGCTTGGGAA
GAGGAGCTGAAATATGAGTAAGAGAGACACGTTCAACTATTCCGAAGCTAGGAAGAACAG
CCAAGCAACCGGCGAGACTCCTCTATGGTATACGACGGCTGGCTACCAGTTGTTCATGAA
GAAGTATTCGAACGAGGGCGAGTCTGTACGCTCTCGCTTCCAAGCTGTAGCTCGCGCTAT
GGCTCAGCACGCCCCTACGGTGTATCCTGAGTGGTGGGAGGAGGACGAATACACTAGAGG
CAAGGACTGGGAACAGGTATTCTTCGACGTTATGTGGGACGGTTTTGTAAGCCCATCCAC
TCCGCTTCTGTCGAACGGTGGGCTCCGCAAGAAGGGCACTACGGTATCTTGTGCTGGCGG
TCTGATGGACAACAACCTCTATGACCGTTACAATGTAATGACAGAGATCGCAGTGCTAAC
GAAGCACAGCCATGGCACAAGCTTTAGCCTGACCAACTGGCCATCAGAAGGAGACGCTAT
CCGAGGCGGTGAGTCTCAAGGTGTGATGCCTGTCATTCGGGACGTTATCAACGTCATGGA
AGAGGTAGCACAGGGTTCTAGGCGTGGTAGCTGTGCATACAGCATCAACCCCCGCCATGG
AGACTTCTGGAACGTTATCGATCACCTGTATAAGCGCACAGAGTCGAACAACGTCGGATG
GCTTCTAGACGACCAGTGGTGCAAGGATATGGCCGAGAAAGACCCGGAGACCTTGAAGCG
ATGGAAGCGAATGATGTTCGTCAAGCTGGCCAGAGGAAAAGGCTACTTCACGTTTATCGA
TAAGATGAATCGGCACCTTGCAGAGCCGTTTAAACGGGCGGGTCTACGCTGTGAAGCGTC
CAACCTTTGTCAAGAGACTGTTCTCCCGGCAAATGACTGGTATACTTTTAGCTGCGTTAT
CCTGAACTACAACCTCGAACTTTACCGTTCTTGGCCTAAGCACCTTGTGTTTATTGGTCA
GGTTATGTCTGATTGCAACATCAGCGAATACCTAGCAACCATGGATGAGGTCAGCGTTCA
GGACCGCCGAGCACTGGAGAAGATTTACCGATTCACCAAGGACTTCAGGGCGCTTGGCAG
CGGTGTTCTAGGGTTCCACACCCTGCTACAGCGGGAGCGATTCCCGGTCGGTAGTATGGA
TGCTATGTTCCTGAACAACGCCATCTTCAAGGGTATGAAGGAGCAAGCGGAAGCTTGTAA
CAGTTGGCTAGCTGAGGTTCTAGGGGAGCCAGAGGGCTGCCGTGGACTAGGGAAGCGTAA
TGCCACTACAATGATGATGCCGCCAACTAAATCGACGGCAGAACTCATGGCTGGGGCTTC
TGAGGGCATAGGACTGGACGTTGCTATGTGCTTCACCAAGCAGAGCGCTGGAGGTGAATT
CTTCCGTGTCAACAAGGTGTTGCTGGAGATTATCCAAGAGCGAGGCCTCGATTGGGAAGA
GTGTGCAAGGCAGATTAACGAACGTAAAGGGAGTGTTCAGCACGTAGACTGGTTGACTGA
CCACGAAAAGGCTGTGTTCCGTACTGCCTTTGAGGTGAGAATGGAAGACTACCTGCGTCT
GTGCTCTCAGCGGCAGAAGTACATCGACCAAGGTCAGAGCATCAACCTGTACTTCACGTC
GAACGACTCGCCGGCTTACATTTCGTACATCCATCGTCTCGCCATGGAAGACCCGAACAT
CCTATCTTTGTACTACATCTACAGTATGAGAGGAGCGGGAGATATCTCCAGAACAGAAGA
ATGTGAAATGTGCATGTAAAATCATTGGCCCGGGGCTTGACAGCTTCCGGGCCTTTGTGT
ATTCTGAGCACATCAAAACAAGGAGGGACTAAAATGAACAAATTCTCTGAAGTGTACTAC
ACTTGGAGCGCAGCGTGCTCTCGTACAAATATCTTGATAGATATGGGATGGACTGCTACA
ATCCGTCGAGACTTCACTGGCGGGATCAACCCTGTATGGGTTGTGGAGTTCACGCGATGA
ACAGCAAACTCCGCAAAGAGTTCGATAAATGGTGGGGACAGGACGAGCAAGAAGAACTTC
GTAAATCCTGTGCCAAAGGATGGGCGGAATATATCTGGATGGCTAGTCGAAGAAAAGTGA
GTGTTGAGCTTCCCGCGCCAGAAGGCGGATTTATCGACATCCAAAAGTACACAATCAATC
GCTGCAAAGAAGCTATCCGCGAAACAGGACTGGAGGTGTTTTAATGAAAGAATATTTTAA
AAAGCGACTAATGCAGACCAGTGAACCGTTCGATTCAGCGACGCAAGCTATGGACGTAGC
GATTGCGTGGGGAGGGGCTAAGTACTGCGAATTCACTTGCGTAACTGCGGATAATCGCGT
CATCAGCATCTGGGAGTACAATGACGTAGAGCCTGATAAACCGGTATTCAATGGACAGCA
ACGAATCTTAGTTCCAACTTTTGAGGAGGTAGTATAATGGAACAAACTATTGATAAATCT
CGGTATATTGTGCTTGATAACGAAGGTCGGCACGTTCTGACCTTGTGCGAGAAGGCCGCC
TATAACCTGCTTAAAGGCGTTCAGTGGACAAAGGACAAGCATGGTCAAATCCATGTAAAT
GGTGTCACAGGCGTTGAACTCGGAGTTCGTCAAGGGATTATCGCTGAAGTATTCCTGTAT
CCGGGCTACATCATCCTAGAAGAGGTAGACTAATGGATATGTTCGATAAATACATGCAAG
TTATGTATGACCGGGACGCTTTCTCAAGGCGAGTCGGTGAGTTAGAAAAAGAGGTAGCCG
AACTCAAGGCTAAGGTGGCTATCGCTAAGTGTGATATGCGTGTCGTGAAAACCCTACTGG
AAGATATCCAATGGGTGTATCAAGACGAAGCTATCTATACTCTACCGGGCTACGTTTGCC
CTTGTTGCGGGGAATCTGCAAACTATGGGCATTTCCAAGGCAGCTTCGATAAGAGCCCCT
GCCTACTAGCTTGTGTACTGAGCCGGAGTGAAGCATATGAAGCCTAATGTTGTAGACTCG
CTAGGCATTAACATGTGCTACGCCTGCGGAGGTAAACTGTACGTCCGAGGAGTATGCACT
ATCTACTGCCCTGACTGCGAGAAGGCAGAGAAAGATAAACCGTTCGTCAGGATGCAGCTT
CTACAGGATATCGCAGACAGCAAGAGGGTTGACAACGAATAGAAGTGCTAGTAAGATGGC
CACATCAGACAGAGGAGGAGCCATGGCTACAACAGAGAAGATCATCGAGTATGTCTCGGC
CTGTATCGAACGCTTGCAAGAGGACGTAACTGGGTACTCGGAGAACTTCCGTGACAGGGC
GTTATCTAATCGCACAGGGTATATTCACGGGCTTATCACTATGGCTGAAATGAATGAGCA
GGTGAGTATCGAGGACTGCGACAAGCTCCGTGTAAAGCTTCGTGAAGCTGCTGAGGCTGT
GTTGAACGTACTGGATTCCAAAATTAACTAGGAGGTGCTCTGATGAATATGAAAGAGATT
GTGCAGGCTTTGATTGTCGTTGTCGCTCTGACTCTCGCAGCAATAGGTTTTGTAGTCGGG
ATTACTGCGTTGGCCAACAAGAGCGCGCCCGCCGAGGATATTGTCGTGTCTACTGAAGAC
GGCACCATCAATCTCGGTCGCTCCGTGAAGAGCGAGAAGCTGCTGTATAGCATGTGCATC
AAGGTAGAAGACCGCAAACCTGCTTGTGAATTCACCACCAAGGACCGTGTTCTTAGCGAA
GTGAAACAGGTTATGCGCCCGGGCGAGGCTACGGCCCGCGATATTAAGATTGCAGTAAAC
GCGGCTGTGACGTATACTGGTGAGAACGGGCAGGAAGCAACCTCTCTGATTACCCCGAAA
GGTAAGTATCTCCCGGCTGGCACCCCGTCTGAAGTACATATTCAGGCTATTGCGAATCTG
TTCGACAGCGTGTACAATGACTACAAACAAAACTTCAGCAAGATGTGAGGAGGACTCATG
AAAGCACAGCCTAAAGAAGTAGAAACCCTGTCCCTCGACAAGCGCCCCAAGGCAGGTGAT
GTTGTTCGACTGCGATCTGACGGTAAAACCTACGAGGTAAATGCTGTATATCCCACGGCA
GTGGGCTGGGAAGCGATTCTCCGCAACATGTACGACAAGACTAAGACCGTCACTGTCAGC
ATGGAGTGGTGAAATGGCAAGAACCGTCAAGTGCGTAAACTTCCTACCTGTCACTCCGGA
CGGTCGAGTAATCGTCCGATTCGGCAAGAAGAGGCTAGGCTGGCTTGGGAAACAGGGTAA
GAAGTGGCATTACTACCCTTACGGATGCGAAGGCAAGATTAAATCAGAGGGGTTTAAGAC
ACTCGCCGAGATCAAGGCTTGGATTCGAGACCCGGAGGCTGTATGGCCCGGTCCGCAGCC
TGAGATTGACTAATTAGCCGACGAATGGTACAATGGTATGTAGGAAATTCCCTACTCAAT
ATAAGGAAACTAGCAATGACTAGCCGCTGGAAAGAGGCCGTGTATCGTAAGTACTCGGTG
CGTCGTCTGCGGCAGGAACTCGAAGAGTTCAAAGAAGCTAGACGCTTTGCACCAAATCAA
CGCATCCGTGCATGGTTTGATGCTCATATCGAATTTCTAGAACGCCACTTGGCTATTCGA
GAGTCCGGCCAAGCCTACCCAGACGACCTTTGGTAATTAGGTAATTAGGTGTGTTGTAGG
TAGAGATTCAAATCATGGTTAGTGGCAAGGACGCCACACTTCAGCCCGCCCACAAGGCGG
GCTTCTTTTTGCCTGAAATTTGCGGTTGACAAGGTTGGCCGGAATATGTAAACTTACGGC
ATCCAACAGAGGAGGGAAGAAAAATGCAATTCAATATATCGAAGCAACTATGGGTAGCTA
AAAGCGAGCGGAAGGGCGACTATAAAGGGACGCTTTTGGCATACATGTCTCAAGTGGACT
TCAATAAAGATGGTAGCTTGGCTGCCAGCTTCAAGAAAAAGCAGCAGACTGGTATGAGCT
GGGCAGGTAAGAATGCCGAGACTTGCTTCATAGACAACATCCCTATCTCGGGGCATTTCA
TCGGGGACAGTGTTGAACGCTGGGTCACTGAGAACAAATATTTCCGTGTGACGGACCCTA
GGGGCTTTGTTCTAGAGGTTCCTACCGGGAATATCTCGACCTTGCTCTACAACTGCACAG
TAATCAAGGGAGTTATTCAAGAGCCTTGCGTGTGGGTTCGGATGGGCTCTCAGCACGTTC
TACTGCCGGAAGGCTCCTCTCTCTACAAAGAAGCTGTAAGTTTCGTAGAGCGAAAAGAGG
CGGCCAAAACTGTTAAAGATATGGTTGCAGGGGATCGATGCACTCTTCTTAGCTATGGGA
CAGAGGAGAAACTAGAGGCTGTCTACCTCGGACAGGTGAAGATCACTTGGCACCTCCTAG
CTAAAAAGGGCCGGTACTCTAGCCGAGGGTGGGGCGGGGGTCGGTGGGGTTACCACCATC
TAGAGAGGGAACTAGATAGAAAAGAATTCACTGAGAAAGATTCGAAGTGGGTTAGTGTGT
TCGCTAAGCCGGTAGGAAAGCGGTGGAGTATCCATTTTATCCTGTCTCCAAAGGCAACCG
ACATTCAATCTGGAAGCGTCCCGGATGAACTTCAGAATCTCGACCTAGTGAATGGTGTAA
AATGGGAGTACAACGAGAAAGACGACCGTCCGTCTTCCTATTACTCTGTGCCGGAGAGGG
TAGTTAAGTGTATTAGTGTAGATGATCCTCTATGGGATAATCTGCACAGCTCCTCGGAGG
TGCGGTGGACCCGCGAATGGACCGTGGTCGGAGCAGAGTGGCGAAAATAAGCCACCATTA
TTTTAGAATTGCCCGGCGAGAGCCGGGCTTTTTCATGGCAGGGGTTATTTTCAAATTATT
TTTAGATTTCTGGGAGTTTCGGCCAATATGGGCAATATGAAATTTCTCAGGAAAAGGCTC
AGAATGCTCGTATAAGAGCCGTTCGAGTGTAGGTATGGTGAGCTACTACTTAGCCGTAGA
AGGACAGTGTAACGCTTAATAGGAGGGTTAGAATGAAGATTGACAACGGTAGCGCGTATG
CGTATTGGCTAGCGGATATGAAAGAGTTTGTTTCTCAGATGGATGCCACAGAGATTGAGA
AGAGGGCTTGTATAGCATACTTAATTAGCTGGTATCAGCGAAGAGTACTGCACAGAATTA
AACGGGAGGAATACGAGGCGATCTAAGCTAGGGACCGGACAGATTTTTAGGGACTGGATA
GATTTTCACAGGCTGCAAAATTAGGAACTGGGCAGATTTTTAGGAACTGGACAGTTTTTC
ACAGAAGGGGATTTTTCTACAGAGGAATTTCCCCTGAAATTTAGCCGGATTTCTCAGAAG
GGCGGATCGTTAGCAGGCTATCTAATTGGTTAGCAAGCTACCTTTAATCGTTAGCACACT
AACTAAAAATTCCTAGGACGGACCTCAGTTGGCACGAACGTTGCAAGCCCGCACCAGTTT
CCCGGGCAGGCGTAGGCCTCGACTATCCGTCGTGAAGGCGATATGTAGTTGGGGACTCGC
ACCCCGCGTTTAAGATGCGCCCATTCTACTATGACAAACCCCTTTGTCAACATAATTTTT
ATGGCACAGTCTTTGCAATTGCAATTAGCGTGCCAACCTAAATCTCAGGCTCCCTCGGTG
TTTATCTCCGGTGCTCGGCCTCCTGCCTGCGTTGCCCGCCTGCCTGTCATTATACACAAG
CTATCAAGACAAGCAAGAAAAATTATAGAGAAAAATCTACATAAAACAGGTTGACAGGCC
TCCTTTATATATGCACAATGACCCCACATTCAACGGAACGGCAGGTAAGCAGCCATGCTA
CATAGTGAAGCCCGGGCGATGGCCCTACAAGAGATGGACAAGTGGAGCCTCCTCGAACAA
GGTTGGAAATTCAGTTTCAATCGCAGGATGAAACGGACGCTAGGCCGTTGTAATCACGCT
AAAAAGCTCATAGAGCTAGGTTACAGCTACGTATCTGCCAATGGAGTGCCTGCGGTATTA
GATACTATCCGCCATGAAATAGCTCACGCTAAGGCGGGACCGGATCATGGTCATGATTCC
GTATGGAAAGAGTGGTGCGGATTGATTGGCGCTAAACCTGAGCAGTACGCCAGCACAAAG
GAAAAAGTAGTCGTCTATCCGTGGCGACTGGCCATCCATCACGGTGGCGGCCAGCTTGAA
CTACTCAACCATTACGGTTTCAGACGGACTAGCATGGCGAGAAAACAGGTACGAGGACGG
CCTGAAACGTTGGGCAGGTTAATTTGGGTGAAGAACGAAAATAAGGGTTGACAGCTTGGG
CCATTCGCGTAGAATGGCCCTCAAGCAAAGGGAATCACCCTGAGCACCCACGCAGGAAAG
CAGATATGAAAGCTCTTTGTAATCATCGGGCACTCTACGCGGCTTACAATCGTAGCGAGC
TTGATCGTATTGCGGTAGATTTGAACCGCTACTTCCCTATCGAGAAAGATGATGAATGGG
AAGACGGCGTATACATGTATCGTCGCCGCACCTATCTGGTGAAGCATTCGAAGGGTACAG
CCCGTTGGCACAGCCTTTACTGCAATGGGACCGTCCAAGGCGTCGGGGTTATCTTTGACG
TGAAATCGGTCGAACCTTTCCGCACAGCATAAGGAGATTCAGAAATGCAAGCTTGGAACG
TATACCAGAATGGCCAGTTGATCGATACTGTTTTCTTCGATATCATGTGTGCTGCCGAAG
AGGTGCGGAAATCGCTGGTCAACCATGACGGGTATCCGTCTGATATCACTGTAGAGGAGG
CCGAGTAATGGATAACCTGTCCGTTAAAATCGTTCTGTCGGAACTCAGCTACAAAGCCTT
GATTCAAGCCTTCGCCTTGCTTGGCCGAACGCTCGACCTAGACGAGGGGTTGGACGATAT
ACTTAATCTTATTGCGGATGAGATCGCTTTCCGCGACGGCCTGCCGCCTTTCGCAGACGA
CTGCGATAAAACAGAATGGATGCTCCGTCGATGGGAATCGCTCCCGGTGGTATCGTACAA
TATCGGGCAGGACGGACCGATTGTCGCTCGGTCTATCGAAGTAAAAACGGCATGAGGGGA
GAACTGATGACCGTCAGAGTAGGAGATATCGTGGCTATCCGGGGCGACATTCGGAAAAAG
CTCGACGGAACATGCCAATACACGCCCGAACAATTGGTAGGTGGTAAATTCCTCAAGCAT
GCCGGCACGGGAGAAATATGGACTGGCCGAGTGAGTAAGATACAAGGTGATATGGCCAAG
GTGGGAGGGGCTTGGCGCGGGATCGAATTCTATGTGCTCATTAGTGAAATAAACGCTTGA
CAACCTGACCCACTAGCTGCAAAATAGCACCCACAGACAACGAAGACGCCGATAGGCGAG
GAGAAAGCAGATGCAAATGTTTTTCAAAACCCGTACCCAAGCCCGTGCAATGGCCACCAA
TGGCCGCAAAGTGGTAGACAACGGCGCTCAAGCTGAAAAGCGCTGGGCTGTCAAGCTGAA
CAAAAAGGAGGCTAAGTAATGGCTAAGTCTGTTAGCGTAGACCGCAAGCGATCCATTCTC
GACGAAGCGAAGGATGGGAGTTTCTTCAGTGTGGAGTTTATCAAGGCGGACGGCACCGTT
CGGAAAATGACGTGTAAACGTCAGATCAAGTCGGCGTATGCTAACGGCCAGTTTGCTACC
CGCAAGCCCACCAGCGCCGGCAAGCCGCACCTGTATACCGCCGCCGAAGTGCTGAACATG
GAAGAGGGTAAATCCGCATTCCGCACTATCAACCTTGAGACCCTGACCCGTGCCAAGGTA
AATGGAATCGAATATAAATTCGATTAAAAGCTTGACAGCCTAGGCCATTCCTGTAGAATG
GCCATCAAGCAAGACAAACTTAAACAGAAAGGAGATTAAAATGGCACGTTCTATCAAGGC
TTCCGACTTCGATTCCGCTTACCAGTTCGAGGCCGCCAAACGTTCCGAACGTAAAGAATC
CCGCAAATCGCGTGACAATCGCAAAGCTAGCCGAGGCCGCGCTTTCGACTTCCGCGACGA
TATGTAAGGCTTGTGCCTAGCCGGTGGGCCGATTCGCCGGCAATCCTAAGCCCATTCGTC
GAGTGGTTTTAGGATTCTCAACCCCAGCGGAGATACCAAAAAATGGTTCGTCAATGCGTA
CCCTGTCCCGATGCTCGCCATGCTTTTTGCCTTGCATGGTTGTTCGCCATGCCGGAGTTC
GCCGGTCTGCCCGCTCAAAAGGACATAAAACTCTTTAAGACTTCGGGACAGTGGGCGGTA
TGTTATGGCTAGACGACAGCGGCTCCCGTCGTGGGTGCAGGACCAGACTGAGGATGATAC
TCCGCTACGGGACAAGATAGACCCCTACCTTAGCAAGCTTGGCGAAGTCGATAAGCTAGA
GGGCCAATTGCGACGGGTCAAGGATGCAACGGACCCGGATGAATACAACGAAATGGCAAG
AGTGCTGAAAGCTCGACGCCGGAAAATAATGCGGCAGAGGGCCCGGGCCTACGGTCTACC
AGAGGAAGAGCCTACCAACGTCCAAGGCTACCACGTCCCGGCAGAAGATTACCGCAGGGT
GATGAGGCTGAAATGGGTCGAGCCTCGGATGTTCTGGGCAGTCCTATCTTTCGTCATTTT
CGTATGGTTCATCTCAAAAAACAGTTGACACGCCCACTGATTACTGTAGAATGGGCGGCA
CGAGAGGGGATAAGCCCCTAGACTGAAAGGAACAACGATATGAAACTGTTCACTTGTGCG
TTCTCCATTGAAAACTGGCAATCCATCGAAAAAGCTGGCGGAGTGGTCTATACCGCCGGC
TGCCTCTGGCTTTCCTACTATGCGAAAGAAAAGATTGTCTAAGGCTTGACAAGTCTCGTT
ATTTAAAACAGAATACATCCGCTCACCACTCACAAGGCGAACCAAAATGAATTTCCATCT
TAACCTCGTCTCCTCCAATTCTAAAACTGGTCCGATCCCTGTCAGCACGTCTCACAAAGA
CACTTGTCCGGACGCTTGCCCGCTCAAGGCTAAAGGCTGCTATGCCTCTTACGGTCCGGT
TAATCTCCATTGGAAAAAGCTAAGCAATGGTGAACGTGGCGTAGAGTGGAAAGAGTTCCT
TCAGCAGGTGCGGTCTATCTCCCGTGGGGATTTGTGGCGTCATAATCAAGCTGGCGATCT
CGTAGGTCAGGATGACGTTATCGATGGGATCGCCCTGATGGAACTGGTGAAGGCTAACAA
AGGTCGTCGCGGCTTCACCTATACGCACTACCCGATGAACAATTTCATGAACCGCCAGCA
CGTATTGTCGGCCAATCGTAGCGGCTTCACTATCAACCTGTCGGGCAACAATGTAGCGCA
CGCCGATGAACTGGCCGACCTGAACATCGCCCCTGTAGTGACTATTCTCCCGATGGACGC
GGAGAATGTAAGCTTTACGCCGAAAGGACGTAAAGTGGTAGCTTGTCCGGCTGAGAAAAG
CGACAAGGTGAATTGTAAGTCGTGCGGCTTGTGCCAAGTGGCCGACCGGGAGTATATTAT
CGGCTTCCGTGCCCACGGCACAGCAAAGAAAACTGTCGATCTCATTGCTAAGGGTTAACA
CAATGGACGAATCTGTTTACGCACCATTCAATCAGTGTAAGATGATGGGCCTAGAACGAA
CCGTGCGGAGCCTAGCGGCTCCAAACGGATGCAAGGTAGTCTTGTATCAGGATCACAAGA
ACAGGCACAGAATTGTCGTATATGACGCTCTAGGGCAGGGTATGTCCGGAGTCATGGTGC
AAGCGGATGGAGAAATTCACTACAAGGAAACTCGCAAGGCCAGCCAAGGGCAGCACCTGA
CAAGGCAGCTACAAGCTCAGCTAACCGCGTGGGGAGTGAAGTGGTTTCCCTCTCAGTACC
AGACGACAGCAGGCGCAGCTTGCTACAAAGGAGGCAACTGATGCGACAGCTTAATAAATC
CCAGCTAAAGGCCTTGGGGCCTAAGATCGACCTAGCCTTAGAGCTAGCTTCTACTGGCCG
GGGCGATCTTATGAATGGCCACGGCTCTACCTGCTACATTGCAAACAAAGCTGGACACAA
CATCTTGCGGATCAGCTATCATAACTCCCGATTCGTTGCATATGGTGCTGAGAGTGTCCG
GGTGACTGGGAATGTCATTGCAGCATGGGCGGAATGGAAAGGGGTCAACCGGGCCGCAAT
CAACGCTCTGAGTGAGTATGGGTATCATCCTAACCTGAGCATAGGGCACAACGATGCTCT
TAAGACTCTGCGTGATATCGAGCGTTACCAGTGACGGCCTCTTACTCGACAGAGAAACGG
GGCCAGAGGTGGGCCATCATCCGTACTGAGCCCGGTTATAGAGGTGACCGGGTGACCGTG
CTAGGATTCTGTATCAATCAAACGCACGCTCTTAAAACGGTAGCCAAATTCCGGGCTTGG
CAAGCGCTAGGCTGGCCCTACACACTGAAAGATGACAGAAAAATAAATGCTTGACAGCCT
AGGCCATTCCTGTAGAATGGCCCCTCAAGCAAGACAAACCAGCCATCAGGAGGCTAGAAA
AATGGAAGCTGTAGCTCAGTTCAAAAGTGAAGATTACGCAAGCGGTGATAGCCGCACCGC
CACCGTTTACAAAGACGTTTACGGCGCATACGTAGTGAGCGTTTTCGGAACTAGCCTGAG
CGAGCCTATTGCCTTCCATTGGGGACCGTTCGAGGATAAAGAATCTGCAATCGATGCCGC
TAAGTCTGCTATCTAACTGAAAAAAGGGCCACAGAATGAAAATCTCCGCTATCCTGAAGG
ACCAGTTGGAATCTAGCTATCAGCCGCATTCGTTTATGTGTGCTTGTGTTATAGAGATGG
ACTTGAGCCGGGACCAACGGTCTGTAGTTATGAAATTTATCGAGGAATTGCTCAACCCCG
AAGAACAGGACGAATGCTTCCCCACGTTACACGCCAAGCTCAGGGCAGAGCTTCCGGGGG
TTTATCATAAGGAGGCGTGGGATCAGGAAGAGTGGGATTTCTTCATTTCCCCATCGGCCT
ACAATATGCGGGTAATATGGTGGAAAACTGTCATTGCACAACTAGAGGAACAAGGGCAAT
GACTCAAGCTAACTGGAGCAAAGGCGTCCGGGTTGTCGGCGGAAAGCGGGATGGTGAGAT
CACACTATTTACCGATGCCAACTTCAGCGAATCTATGGGCCTCTGCGGTGGTGATCCCGA
TTTGATCGAGGCTCTGCCGATGCCTGCTGCACTCCGCCTGCTGGTGAAATGGAACAAACT
GTGCCGCCAAATGAAGCTTGACTATCGGTACGAACTACTGTAATCTGCAATCTCCATCAA
AACAGGAATGCTCCAATGTCGATCTTAGCCTTAACTCTGACCCTCTGCTCTGCTGTCCAA
TGCGATGATTACATCATCGACCACACACTCCCGGGCCGGGCCGCAGAGTGCAATGCTCGG
CTGGTAGAGGAGGCCGAGGAGTGGGGCGACGCATGGGTGGCGACCAATGCCGACGCCCGA
CTGACCCGCTACCTTAGCCGGTTCAATATTCAAGTTGACCCTCGCTTTGTTTTCGACTAC
GATTTTACCTGTCAACTGATAGCAGAGGATGAACTGCCATGATCGCTCCACATATCACCA
ACAAACAATACCGCCAACGTGCTGCTGTCATCGTCCGCCGAGTGAAATACTATGAAAGGC
GCTGGCTCCGGACCGTAGAGTTTTTCTCAGGTGCTGCCGCTCAGAACATCGCAGAACGCT
TTGCCTACGCTGGGCATGCTGAAGAGCAAATGTCCAAGCTCAAGGCTGAGTTTGCAGCCT
TCCCGGTGGAGGTGAGGCTAGATGCTCTAGGTTGGATAAACTGGAATACTCATAACGACG
GTCTGCTAGAGGAGTGGATTCATCAGGATAGTGACCGTGCCATCATGCTAGAGTATGACA
GCGAATCGAAGCGGCCTGTAGCCTGCTGGGAAACCACTGTCTATGCTCTTCCCAAGCTGG
CAATGCTGTAAAATATCGCCCGTTAGGCTGAAATAATGCTTGACGGGCGTTTTTGTACCT
GTAGAATGCCTAGCCAAGGGGTGCATAAGCGCCCCACCTCAAAGGATACAGAAAATGGCC
ATCGTTGATCGAACCGCAGGTAATGAAAGCCCGAATCACTGCTACATCGTTACTGGCGGC
ATTGGACCAGAGTTTGAGCCGGCCCTGTTCACTACCCATGCGCTGGCAAAGAAGTATGCT
CAGCAGATAGTGAAATACGGCAAAGAGAACGACGTTTTCCCGCCTTTTACGCCTGTTATC
GAGACTCGCCACAAGGCGGATATGCATCGGTTCACCATTCACAACAAAATCCCCGAATAG
GAGATATATCTATGAAACTTTCTGAAATCTTTCAAGCCGGTCTCCACTACGCAATTTCAG
CCGGGATGCCGTGGTATATGTGCATACGTCTGCGGGAGTTAGACCTGCCGGGGAATCTCG
ACGGAGTGCTAATTGACATTCAATATATCATGAATGAAGCGGGGATAACGTCTGACGGGA
TCGACGACTACTTCCGGGAAACTGGTATGGCTGATGCCTATGTGATGACGATGCAAGAGG
CCGTCGATTACGGGAGTCCAGCTTACGCCATGTGGGTGCAATTTTGGTTTTTCGTAATCT
TTGACTTAGCCCGAAAAGGGCAGTAGAATAGCCAGCAAATAATGTGATCTAGAGGGACGA
TCATGGGTGACAAGACAGGCATGAATGCCAAGACTGTAGTTTATCGAATCGAGTGCAAGA
ACGGCGAGGGCCCTTATGTTAACGGGTGCATGACGAATTCGTCTTCCACTCCAGAGCGGC
CTGTCCCTGAGCATGACGGGATACCAGATGTTCGCCATTTTGAGTATTTCGCCTTCACGT
CCCCTAGGCAGCTAGACGATTGGTTTGGCGATGAATACGATGTGCTGTCCTTCTATTCTA
CCGCACGGATCAGAGTCTATGAGGTAGACAAGCAACATGTAAGAGTCGGCATCCGGCAAT
GTGTATTCCGCAAGGCGGAAGCTGATTGCGTCCAAGAGGTGAGCATAGACCAATGGGCTA
GCATGGTACGCGCCGGACTATTGTCCGCCTCGGTCACACAAACTGTAGCTCCACGTCCTA
CCGAGCAATTGTCCCTAGACCTAGGGGATGACTCGGCCGGTGAGGCTTCGAAAGAAATTT
TGAAAATCTTCGGTTTGTAGGGTTGACAGCTTGGGCCATTCGCGTAGAATGGCCCTCAAG
CAAGACAAACCGCCGATAGGCAAAACCTAAGGAACTATAAGCCATGACTACCGAAAACCT
TTCCTCTAAAGAAATCGAAACCGCCGTTGACGAATTCCTGAAGTCAATCGATGTTACCGT
GGCCACCGTGTGTCGAGGTGAGCGCAATAGTGAAGGTTGGGTGCATGACCTGTGGGATGT
AAGTTTCATCCGTGGCGATGGCCTCCGTGGAGTTCTCGACACTGAATTCCGCACAGGCGT
CGGGCATCGCAAAGCCTCGATTCCCATGCCCGCCGACATCCGCCGTCTGGCCCCTAACAT
CCTGATGCGGGTAGAGTGGGAAAAACGTTACATCAAGGCTGTAGCACCTGAGGCTACCTG
TATCCTGTCCAGCCTTATCCGGGACTCTGAAGCGGGCGGCTACACTTTCGAGGAGTTCTG
TGGAGAATTCGGCTACGATACTGATAGCCGAAAGGCCTTTGACACTTACATGGCTTGTCA
GAATATTCTGAACAAGGTGCGGAATTTCTTCACTCAGGAAGAAATTAAACACATAGCGCG
TCTGGTGGATCGATACTGAAAAAAAAGGTTGACAGCTTGGGCCATTCGCGTAGAATGGCC
CCTCAAGCAAGACAAACCACAAACCGGAGACTCCCCATGAACAAGCCAATGAACAAGCCA
ATGAACACTTTCGCATACGCCCACTACGTCGCCCGCATGGACAAGGCCTCTGGCACTAGC
CTGCCCTACGCCACCCTGTTCCGCATCGCTCTGCGTAACGCCTACCGCAAGCCGGGTGTC
CGCCAGTACGATTGTGCAGCACTCGTCGAGGATATCAAAGCGCGTGAACTGGTCCAAAAG
CGCATTCGGGCTGAAAAAGCAGTAGTGATGGCGGCAGTAATGCGTGCAATCCGTACTGGC
TTCACTGTTAGCGTGAAAGATGGCAGCGATGGCGAGTGGGTAGTGAAAAAGTCTACCGAT
ATCAAGGAAATCTCTGATAGCCTGCAATCTACCGACGAGGATATCATCCGCTTCCGCAAG
GGCGACGGCCCGGACAACATCGTCGGCTCTATGTGGGCAATCTACGGTAACAGCGCCGGC
GAAGTGATCGCCGACTGGACTGATAATGCCACCATGGCCATGATTATGGCTCCGGCTGAG
CGGAAGATGGAAAAATACGCTGAATTAGGTATCTAATGCTTGACGGGGCCCGAAAAGGGC
CCCATAATGCACCCATACCAGCAAACAACATCAGGACCGCCCCGATGAGCCAGATCAAAA
TCCAAGAGTATATCGTAGCCGAGATCAAGGTTAACCATTGCGCTCACCTGCGCAAGGGCC
AGACCATCCAAGCCCGCGATGATCGTAGCAGTGAAAAGTGGATTGCATTCATGGTGGAAC
GCGGGTATAGTGCCTCTGAAGCCGCTATCCTGTTCAAAGACGCCCATGATGTGGCAATGC
TCGAACTGGCAGCCTGAAGAAAATCCTACTAGGAGTACTGACAAATGAAACGCCAATATA
CCGACTCTTACCAGTGCAAAGCAATCGACGGCATGATCTCTATCCTCGGCAGCAAGAAAG
TTACCACTCAAGTGCCGCCGTCGATGAAAGCCTCTCGAAATAAACTGCTGAAAAAGATGA
TAAAATACGTCGCCCGGGCAGGATGGAAACCGTAATCGACTAATTATCACAGCCCCGGCG
AGCCGGGGCTCACAATAGGAGATAGAAAGCATGAATACCCTGATTATCCGCTTGAATTCC
GCCTATAGCGTTCAAACTGTCCGTAGCTTCCTTGTGGAGGCTGTAGAGAGTATTCAGAAT
CATATCCGCGACTATGCGACGCATACTGAGTACGATTCAGGCTTGAGCCCGGTAGCACAC
AATCACCGTCTCGCAATCGTCACCTTGGACGAAGATTGCCGAGTGTTTCACATTCAATGC
GAGTCACCCGATCTTTATCTGGCTGACCCTCGAATCGTGTTTACCGATGTTGAAAGCCCG
GCATATACTATGCCTGCTAACGAGATCGACGAGTATAGTTACATGACCGCATGGCAACAT
ATCCGCCAAAAAGATATGACAGCAAAAGACCACCTATCCGCTTGGGCGATCCTTTTCGCC
TCAGAGGAGAATGTCATTGATATTGACCCGCGTCCCAACCTGAAAACGCAAATCTTGCAT
TGACCGAGGAGGGTCATCAAATGAATAACAAGTATAAGGATAACATCATTGCGGAATACT
GCCATTTCGACGGCAGACGGCCAGTCCTTTCCTTGGCGGCACGTTGGGTCCACCATGACA
GTCGGGTTGTGAAACTGGCAGCGCTCTTTGCCTGTGCTGGCTATGGCCGTCCTTACAAAG
ATTGTATTCGTGCCTACAAGGCCGCCAAGCAGCGGGAAAAGGATATGCGAGACCCCGCCA
GATGGAAGCTCAGCCGAGCGACTAAGCGGGCTCTACGGGCCCGGCTGGCGTCCAAAAAGA
AACTCAAATAAACTGTTGACAGCTTGGGCCATTCCTGTAGAATGGCCATCAAGCAAGGGG
ATGAGCCCTAAGCAGTAGAAGAGGTGAGAACGATGCAAGCGACCTACCAAGCCCTGAAAA
CCCTCCGCGATGCTTGCGAAGCTGCCAAGGATGAAAAAGGCACCATCAACGGAAACAAGC
TGAACTCGCTTCGCAATAAGGCTGTCAAGGAAATGCAAGCTGGCGGCGAAACCTACTCTG
ACGCCATTAGCATGGCTCACGACTTGATTAAGAAGTATCGCAAGTAATCAATCCGTAACA
AGCCCCGGGTGACCGGGGCCTCTCATAGGAAAGAACATGGACTACGAATACGACGAAAGC
TGTAACGCCTATGGCCCTCCACCGCACCCTGATGACGTGTACAAGCCCAACGCTGCCGAT
AAAACGCGTTGGTCCGCAGTATGGGCACGTATGGCGCATGACCGTAACGTGGAACGTGCC
GCCGAGCGCACCGAGGAAAGACCATGAAAAACGTTCAAATCTGTTGTTCGAAGTGTGGAG
CCATCAACGGCGGTAACAGCAAACACGATTGGGTTATCGCATTAGGTAAGAAGTGGTGCG
GTCATTGTGGGTCCAATCTCGCAACCCTTCGTCGCTAACCATTCACTGAAGCCTAATCGA
GGAAAGATCATGAACGCCACTCTGGCACGCCTACAGAACGAACGCTGTGCAACCCTCCGC
CTGATGCTCCTGCCGGAACATCGTGGGTATCAGTTCTCTGGCCATCCTTGGGCCGAGCGC
ATCCGCAATCTCCACGATGAAGCTAAGTGCATCCGCCGCCATCACTAAACCGCACTAATC
CATTAAAGCTCTAACAGCCCGCCTTGTGCGGGCTTTGTTCTATCTGCGAAAGCGGAGCTT
TCAAGTGTAATTAACATGTAATGTTGTAACACTCTGTAGAGGTAACACCAGCATGACAAG
ATAGACCACTTGTAACGTTAACGTTACATGTATAACAGCATGACAGCGAAAGCTGTACAG
ATAGCATTCTGTCCAGTTCACTCCTATCAAATAACCCCACAATAACCCACCTAAAACCTA
TCGGTTTTAAGCGGCCTGTGAGGCCGTATATGAAGCGATAGCTTCGCTATGTTAGTCTGC
ACCGTCAAGGCGAAAGCCTTACAGTAAGAGGCTTGTAGGAGGTTTTAGGGCCATATGCGA
TTAGCATAGGATGTTGGTTGTTTTAGGTAGATTTTTGGTATAACCCCTTTCCGCGCTACC
ATTGTCTGGCTGATGTTAAGCGACAGCTTACAGCCGCTCTCATTTTGCAATGAGAATTGC
GTACAGTTCATCATGCGCCTCGAAAGTGACTTCACTTTGCATGCGGATGCATGCTATAGT
ATACCCTGTGATAGCTCCGCTATCCCGTTTTGCATGGCACACGAATTGCATAAGCATGAC
AAGGGTAACCCTCGAAAGCGGAGCTTTCAAAGTGGTCTGTGAAGTGTAGGCTCTACCTCT
ATTATATTTGCGACCGGAAGTAGTGACACTGAAAACATTGACTCTAGCCCAGTAAATACG
TTAAACTAAAAGGTTGATAGCTGCCGCTATCGAGTAACCAACATTCACACAAGGAGGCAC
AATGACAAGCAACAATCATGCCAAGATTCACATCCATTACGCTAAGCAAATCTACTACGC
CATGCGTAAACGTGTCATCGCAGGCGATGAGATATGCAAGGAATGGGCCAAGCCCAAGAC
AGGCTTTGAAGCGTTCTATGATTGGGTGGTGAATGTGTCAGGGTACGCCGTAGGCTACCG
CCTAGATAAGAATATCCTCTTCCCGGGAAACAAGCTCTATGCTCCGCATACGTGCGAGTT
CACGCCTACATACATGCATACACTCTTCACCAACTGTGACAAGACAAGCAATAACCGTGC
CAATACGTGGGATAGCTATATCCCTCTAGGCGTCCGCCTAGCACGCTATGACAAGTACGG
GATGCCTGTATACGTTGCTCAGTGTAGGACACTTGGAAAGCAGTCTACCCTTGGATCGTT
CGACGATCCGATGCAAGCCCATGCGGCATGGCAACATGCCAAGATAGCAGCCATCCATGA
ATGTATTGATCTCTATCAGATGGAGGATGTTCACAGTGTGAACGTTGTTCAAGCGTTGCT
TGAGAGAGTGGACAAACTTGGCAAGGATATTGCACAGGGAAGAGAGACAACCCTTTTGCA
ATGATTTGATGGGGTCGATGGTCAACCCTTCGAGAGTGCGAGAACTCTTGTGCGGGAACT
CCGATAGCGAAGCTATCAAACGTATATGCATATTCCATGCCAACATGTGGAAAGATTTCA
CCCGGCAGGGCGGGAGCCCGTTGGCATTAGTTTTGCATAGCTTAGCTGTCGCTAAGAAGG
AAAGACCACGGCCACCCATTATTTTGAATTTACTAGGGGTTAGGAGAGGACAGTGGGTTC
GTGGTTAAGTAGCTCGGAAATAGAACCACAACTCCCGAACAGGATACACTCGGACGTAGG
GCCACACTACATCATTATTTTCCCAGCCATAGAGTAAACGCACTCTTCAAGTGGTCATTA
TTTCTGGGAGGGGGATTGGGGTCGTGCATACAATAGCAAGCTCCGCTTGCACAGGGAGAA
TGGCCAATGCAAATCTATAGGATTGGTATTGGGAATAAGCTGACAACAGCTAGACCGTAA
CTGTGTAAAAGAGAGCCCCGGGCAGCAAAAGCTGCCCGGGGCTTTTTGTCTCTACTGATT
GTATTCCATGTCCTCTGCTTCCAAGCATCCCTGCATAACCTTGCAGTACTCTTCGTTAAT
CTGGTCCATGTACTGCAACGCTCGTTCTTTGCTCTGGTCAGGACTGAACCCGTTGTGACG
GAGAATCTTGTTCAAATTCTCATCATCCCAGAAGTCTCTGGACCAAGCCTCCCAAGCCAC
TGCTTTCAGACTCTTTATGCACCCGATAGGGGCTGGATACCAATATTGCTCAGACATTAG
ACCCTCCCGAACGTTACGATCACGCCTTGCTTGGCTAGGGTTGCTTGTAGCTCATCCAGT
GAGAGGGGCTTACCGCCCACGATGACATACTTATCTGCCTCTCCTAGAGTCATCACACAC
TTAGTCTGGAAGGTAGGCCTATACAAGGCGTACATATCGGGTTGCCACTTGCTTCGCAGG
CCTGTTCCGTAGCTAATGAACCGGTCTTCGGAATGATCCCGGATGTACAGGCACCGGGGA
CTATCGTCTGGCCCATCTACGGAGATAGGGTGTTCCATCCACTCCCTGATCTTATCAACG
ACGCCCTCTCCTACCTTCAATTGAGCGTACTGACAGTACTCAAGGAACGCATCAAACTGT
TCGGCAGTGAAATTGCTGACATCGATCTCTTTAGTTTTCATTCTCTTCCTCCATGTCTGC
AATGATTTCCTTCAACGCTTGAAGCGTGTAGGTGCCATTTAGCTCTGTAGTAGGACCAGT
GTCACACTCATGAAGCGTGTACGCGGTCACTTTCTTCTCAGTGACGAAATGGAAGTCTAC
CCTTCGGAAGTCGTAGGTCGATTTCGAGTATCCAATGGCCTCTTTGAGTTCTTCCTCGTA
CCACAGGAATGTCATTGGACTTCCGTCGCTTTTACGAGTGTAGTCCAGCAGATTATCTCC
AACCCACAGTAGCACTTGAGCCTTGTCGTACATGAAGCCGTTTCCGCTCCCAAACTCGAT
ACCATAAGCCTTAAGGTGCTGTTGTACTGCAATGTTCTCTTCGGGGGTCTTCACAATAGT
GTAAAACTTCACCAAACGCTCTTTCAATTTCATTCCTCCTGTCTGTGTTTTATGGGAGTG
TAAAACGTTCCCAGTCCATTGTCAAGCTCTCTGTGGGGATAGACCGTTCCAGACGCTCTC
TAGCCTCTTTTAGAATGATATCCCACTCATCGCCTTCTTGGACTCCTACATACTTCTCCA
TCATCAACGCACGGTCAATCAGACCAGCCTTGTAGTTATCCCGGCTACGCTTGCGGGCTA
CCAGCTTGTCGATGTAGTCCTTCTTATCTGCCAAAGCGGCGATAGCCTTCCTAGCCATAA
GGGCAGCGTATTCTTTTTCCTCCGAAGTCATCCACGGCATATTGGTCAGTACGAGGTCAG
CGGCGTCCCTGACCAAGGGGATATCTCTCCATAGGAATCCTACCGGTATCAGGTCTGAGT
CTGCGGTAGCTCTCCACTGTAGCACTGTTCCGGTCTTAGAATCTGTAATATGCACCTTCT
CATCGATACGGTGGGCCATCATCGAAAATCTCCAGTTGCGGTCTACGATCCATCGCTTCC
AGTCGAAGAATGTAGCACAAATAGCCTTAACCGGGTCTCCGACCTTAGCATCCAGTTCCT
TCAGACGTTTAATCTGCTTAGCGTTTATATACTTTACAGGCCGGGAGGCTGGATCATAGC
ACTCCTCTACCTGAAACTCCTCAGCCCGAGACTCCAACACTTTAGGCTCATCGTTACTTT
CTTCCTCTCGGAACCATGCGAACAGCTTCTTAAACATATTCAGTCCTCCTCTCCTGTGAT
AGCTACCCTAGTCAGTCTCCACACTTTTCTAGGGAGACTAACGAGGAATAGCATAGCCAT
AAACGGTACGGCGATAGGCCAGAATATTCCAGCTAACACCGCTCGGGCCTCTTTCTTACG
CCTCTCCGTACTTTCTCCCCTCCACCCAAGAAGGGGGCGTAGAGATTCCCATACGAACAT
CCCTATCACAGAGTAGAACAATAGGCAGACCACTCCGACGACTATGCATACGAGAATGAG
AAGCCCTACTTGTTCCATGGTGCGATCCTCTTAAGTAGTTTACGGCTGTTCCAGATAGAC
AGAGCCCACTTAGGTAACAACACCACTTTCAAAACGCTGTATGTTGCGTAGAGTAGCAGA
AAGCTAGCATACACTGTGTATGTAATAGGCCATAGCAGTCCCAGTACAATGGCAAAAGCC
TCGATACCCTCTCTGTGATCTTTAGTCGCACTAGCTGGGAAGTTATCTTTGAAATACTCC
GTGATACCCATGTAGGTCCACATGAACACAATTCCCCACGCGAGCGAGAGGACGAATAGC
ATTGGGATTATCTCCATTACTCCCCTCCTTTTCGAATCAAGTCGAAGTAGGCCCACACGT
AGAAGTTGCACCACGCTCTGCCTATGGCCCAGCCTTCCCAATAGTCTTCCCAGTCCTTGT
TCGGAGGGTTATTCTCGAACATGGATAACACGCATGAATCAGCTACTTCGTCCAAGAGGT
GCATTACCGAGTTAAGCGGCCCGATTCCAAAGGTCTTATCTAGCCACCCCTTAAATCGTC
CCACGTCTGAGACGGTGACGCTGGAGTCACTGCACAGTGCCAATCTCTCCAGAATGTAGC
ACATGTATGGTTCCTCTTCGTTCTCGAAGAATGCCCGCATGCCCTCCTTGATTAGAGGTG
CAATAGAATTAGAGTTCATTGTTTTCCTCCTTTGAGAGCCCCACTAGGGCTCTAAATTTT
TGGTTCGACCTGCGGTCAACACATCACAACCCAGTTGTACAGGTCAGCCTCCTCGTAAGC
CGATGGGTAGCGATTGTACTCCAAGAAGAACGCATGGTCAATAGCTGCGTATTTGGTTGC
GAAGCTGTTGATGTACGTTATAAACCCGTTGTCGCCCCGGCTAAACAGGGCGTATTCCTC
AGTCCCATCCTTGAACGTATCTAACGCGATAATATACTTGTCGAAGATCACAGCATTTCC
TCCCGGCTGATGTAGTAATTGTATTCGTCCTCTTGGACGAGGAACGCACGATAGAAGCCT
TTCTCAAAGTCTCCCTTGACCAGCACCAGATTGCCCTCCACCTTGCATGTATAGATATAC
ATGGTGTTTGCTACTCGGATAACAGGATCATCCCCGGGACGAACATCCAGAGCAGAGAGC
ATAGCCTCCATTGGCATGCTTGTCAAGTGTGCAACTACAGCCGAGCAGTATTCTGCATCG
CGGCCAGAAGTGTCCTCTGCATAAGCAGGAGCGACCCCGCACGAATGCAGGGCGAGTGTA
AACGCAATAGCTACGAGTTTCTTGAACATACTAACCCTCCTCTTGGTCTGTCTGGGGAAC
ATCCGGCTTGGGGTAGACGAATGTCTCTGGATACTTCATGCATCTTAGCTCATCTTTGCA
GAACCCGTCAATCAATTTCTCATGTGCGAATTCCGCAGCCTTGTAGTAGCGATTAGCCTG
TCTTGCCTCCACTCGAAGGAGGTGGCTTATCGTGTGAGCGTTGATATTACTAAGACTTGC
AAAAATTGTCAAGAGCTTCTTTGCAGAGAGGGGGATTGACCTTTCGTCAGGTCTGTACCA
ATCAAGTCTGGCCACTCCTTGGAGAACGTCGTCCACCCACAGAGGGTAGTCTCCATCAAG
CTCGACCATGACTTCGTACATTTCAGGCTGAACAGCGTTGAAAAAGAAAGAGGGCTTTCG
CCCTCTGCTGGTATCTAGAGGAGGAACAAGCCCCCTCTCCACCTTGTGAATCAACCGATC
AGCATTCGCCCCTATCCGAGACTTTAGCTCCTCGATCTCTTCACATGTCAACATACGGAG
GGCACACCTTAGATTTCTCGATAGTGAACACGCCTTCGCATGTCAGGTTAGCAGGTCCAT
ACCACTTGAACATGTTGTTGATATCGTATCGAATCAACTGGCGGAACTTCTGCCCGTCGC
ACATATCCACGTCGAACCACGCCACGACCCATCTGACGTAATCTTCAGGGTCGTTCTCTT
CTCGCCATCTGCACTCTAGATCAACGTGACTGGGCTCTAGAACGTAAATGTCTTCGAACC
CTGTACTCTGGTTGTTCTTAAGTCGGATGATTCGAGCGCGAATCCATTGCATGCACCTGC
GGTTTGGGTCTGTGATATCGCAGACTACCGGAACCTCTGGAACCTCAACTGTATACATGA
TTGTACCTCTATTTGTTTTACAAGAACGCCATGATTCTGTCTCTGTCTGCATGGCGGTAC
AGATTGAAGCAAGGCACGCCTCTAGAGGACGCCAGTTGCCAAGCGGTCCTAGTCCCGCCT
TCTGGAATCCCGTTGTGGTCTGTCTTGCTCCAGCACACCAAGAACTTGCTAGGCGTGTTG
AGATCGATTCCCAGCACTTGGAAAACGTTCCGAGTGTGGGCTCGTCTTGCGAATGGACTC
AGAACAGGCTCTCCATCATTTCTGACAGAGTTCCAAGCTGGGTGTATCCGGCTAGCAATA
CTCCCAGCTTCCTCCCACTGCTCCGGCCACTCTTCTGGAAGGATTGTAGCTCCTAGAGTC
TCAGTTATCACATACTGATTGAACCCGGGCTCCGGCAGATAGATTTCTGCCGTAGTCCCT
CCTTTGTTATTGGATATATAATCCAGCCAGCCAGCCTCGAACGCTGAATCCGCTCCCTTC
GCTCCTCCTGACCGAAGAATAAAACCTCGTTCCGCTAGTTTACGACCTAGCTCTCTGAGA
ATGCCCCACTCCTTGTTAGGCGTATCTCTACTTCCGACTCCTGTATAATATCTCATCGTC
TCACCATTTGTCCCAGTTCTCCGTATGTAACCCAGTGGTGACGATAATCCCCTTCTTGGT
TGGTGTACTCCACTAACACCGGATAGAAGTTGTCTTCGTAGTCTATACTCACCACCTTGC
ATTCACATCCTCTGGGAACAGTGTATCCATCGCTGCTCATGGTTAAGAGCATTCCCCGGT
ACACGTCTTTTATCTCTAGCTCACCTTCTCCAGACTCTTCAAACTTGAGTCTCAATTTCT
CGATTATTTGAGCTTGCAAATGGAGAAGTTCGTACGCTGTTAAGTTGTCCAGATCAATAG
ACATGTCACTCTCTCCCTTCTAGCTTTTTGAATTCCTTGTCCAGCTTAGCTAGATGCTTA
TCCAGCTTATCTAGCTTTTTATCCTGATAAAGCTGGAAAGCCTCCGAGCCCGGGGCCAAC
CAAGTCCCCTTATACTTAATTGCACCCCGGATCATATATCCTCCTATTACAGTTCGTAAA
CGTATACCTGAAACTGGCCGAGCGTCTCCTCGATAAGAGGCTCGACATTGCTCCAGTCCT
CTCCGGCTAAACCGCAGCCAATCTTCGGCAGACCTACACACAACTCCGAGGTAGGAATCG
CCCGAGCGTCAGCCTCTGCCTCAAGGAACAGAGACATAGTGTACAAGGCTTGGCCCAGTG
CGTCAAGCTGAGTGTAGACTCGACCCTCTTCTCGGCCATAGTTGTACTGTCCGTACAGAT
TATAAATCGATCCGTACTCGTTCATTGTGAAGGTCAGGGAGCCGAGTTTACCTCGGTCCC
CTTTCACCGTTGCTTGGTCTGCCTCGTAGGCCTCCGGGAATTGCTCCCGGATTTGCTTAG
CAATGCCGCTGCCCATGGTGTTGAAGCAATTAGCCTGATGTGCAATAGCGTGCAACTCTT
TGTTTTTGAACGCGGCGAGCAGGTCGCCCTTTCGGTATTTAATCATTTGAACCTCTCTAC
ACTTGTGTATCTGATATCCAAACCTCTCGCATCGGCTCTGATGTTATCGTACATCGAGTC
TAATGCTGCGAATGGAGACCAGTACCCTTTCTCCCAGATTTTAAACTTGAACAATCTATG
TCCTTTATTCTCTGGTCGGCCTTGCTCGCAGAAGGTTGTAAAATAGTATTTAGCCAACAT
CCTCACCTCCTCTTATACGCCGTTCTTAATCCGATAACGGATAGATTCCAGATCATTGTT
CTTCAAATCTTCCGTGTCCCATTTCCCGTCTTTGAAGAACGTCCGCAAGGAACCAGTAGC
CTCCTCCTCTTCAGATACATTATCTTTCAACTGGAGGAATCGCCCATTTTGCAGGTCTCG
GTACACTTTCAGGAGACCTTTAGCCGACTTCTTCTTGCTGTCAGTTTTCGGGTCTTTGAA
GATTGCAATGCCTTCACCGTTCACTTCCGTGTAGGTAGCTTTCACTGCGAATCCGAAAGT
ATCCCGGGTGTTGCACTGGTAAGTGTAACTGCCAACTCCAAACACTACGTTCAGGGAAGC
GAAACCTTTCTCCTTCAAGCGCTCCAAGATTTTAGCTGCTCGGCGAGTCGTAATGGAATC
CCCATAGATCAAACCGATATGAGAATCGAGCATCTTATAACCTGCTTCATTCACAGTACC
GCCGAAGGTTTCCCACAGAACTTGGATAGCTCCTTTTACCTGCGCCTCGCTCACTTCTTC
GAGTTTGTAGCCAATTACATACTCGTATTGATCCCACTCAGTGGGAATAACCTTGAAGTA
CTTATCTTCAAAGTTCAGAACAAATGCATCGTTTCCATTTACTGCATCCATGATATCCCA
GTGTTCGAGCGACTCGACAACTTCAATATCACCCAGTCCGCAGATAACCTCTACAGGATC
ACCAGAGTCTGGACGAATAACCAGCTTGCCATCACGCTCCATGATCTGATCCTTGATGGA
CGGCAGAATTTCAGTAAGCACGCCCCAGAAGTCGAAGCTGTCTGCCACGTACGACAGAAT
ACCACTCGGGAACTTACCCATCAGTTCGATAAGGAATCGACGTTCTGCTTCCTCACGGCC
ACCTTCACCTTCCCCATCCTGCATTTCTTTCTCAATACGGAGGATGTTGCTAGTAGCTAC
AGCGTGTTCAGTCGCAGGTACGGAGCAAGCTACCAGACCAGTCGCGCTATAGTAGTCCTC
GGCGTAGATAGCCGCTGCCAAGGTGTCCGAGCCTACGAACGAGGTCAAGTGACCAACACC
GCTTCGGGCCGCGTCTTCAATCCCACTCATACCCCGGGCACTGAAGTCGTGAAACTGAAC
GGACAGCAGGAAGTCGTCGTAGCACCCAGTGTGGTTCGCGTAGTACCGGCCCATAGCTGC
GTATTCTGCGGCAATGGTTGCGTTGGTTGCGGTCTTCCAAGTCATAGCCGAAATCAGGGT
CTCATGGTAGTTCACCAGCCAGTAGAACTCAGGCAGGGTATTCTTGATAGTTAGCACAGG
CACGCCCATAGGAACGCTAACGCCCTCGGTCAAAGACTTAACCTCCAGAGGCAGATAGCC
GAGCTTGTGAAGGGCGCGAAGCTGCTCCACGGCTTCCATTTCCCGGCCAAAATAGCCAGT
CAGGAACCGGGAATACCGGCCAACGGCGTTCTCTTCTTCAGCCGCGAAGAAGCCTTCCCA
AGACTCTACGATCTCCATCCAAGCGGCCCGTCCGCCGACCCACTTGAGAGTGCCGTCGTA
GAAGTCGGTGGCATGCGTCTTTCGATAAATCTTATCGGAGCGAGGGGTCAGGTTGCTATA
GACAAGCTGAGTTCCTTCCGAGTACATGGAGCCGTGTCCAATCTTGTACCCATCAGTGGA
AAGATGCGGCAGAATTTTCATGTTATTTCCTCCTGTTGTGTTGAATAGGCCCAGACTACA
TCAATCTGGGCCTCCTGTCAATATTCGACTTCTTCTACTGGCAGCGTCAGCACAGGGTAG
ATGCATGGAAGGCGCAGCAATGGTTGCTTCTTGCTGTGGAAGTCTTCCCAAAACTTATCG
TGCATCGTCTTGTACCGAGCGTCCAGTTCCTCCTTGGTGTCGAACTCCTCCACCTTGACA
ACCCTCTTCTCTGGAGCCGAACGCTTATACTCTACAATCCAGATACTCAAGCTCACAGGC
GTTCCGCCTTGGAGTATTCAACCGGAATGACAGTCAGCGCTTCGTGCTCTTTTTGAGGGA
GCGTATCCGAGGTGTAGATGCATCCGAAGGAATCCAGCAAGCAGTCGATACCCTTACTGA
AGATTCCATGAGTGACGAACAGGTCAGGGAGCACTTTACTGTACTCTCTTAGGGCCTTAG
ACACGCTTCGGAATGTAGCACCTCCGTCACAGATATCGTCGATCACCAGAAGCGGAAGTT
CAGGATCGATATCCGAAGGATTCAGAATTTCAAAGCCTTCGATTTTACCGGTCTGGAGAT
CGCGCTTCTTATTGCAACGAATTACAGCGGCTGCTCCGGCCTTCTTTGCAAAATCCTCGG
TCCGCTTTGTTGCTCCGATATCAGGGGCTACGAGTTGATATTGCTCCCACTCTGAACGGC
TCAGTAGGCCACCGAAGACCGAAGATTGACGGACAACTTCGACGTTGTTAATCAGGTGCA
GAGTTGCGTCGCTGTGAGGGTCCACAACGATAACTCGGTCATATCCGGCAGAGTTCAGCA
TGTTTGCAAACACTTTCAAACTGAAGGCGTCGCCCGGATTGCACCGACGGTCTTGGCGAG
CATACGGGACATAACCCAGCAGAAGAGTCATGTGCGGATTAATCATTTCTGCTCGGATCG
CCTCGGTGACGTTGAGAAGAGCCATGATATCATCACTATTCTGAATCTGAGCTTTAACGT
TTACGAACGCATGGCTTTCGATGCCGCCCACATTAACGCCCACTTCGCCGCCCGGGAACG
TCCACACTTTTGCCTCTTGCCACGGACGGCTCATAACACCTTCCATTTCCTGAAGGCAAC
CTACAGCATAACTAATCATACATCCTCCTCACAGTTTCACAGAGAAGCGATTCCGCATTG
CTTCCATCGTGCTCTCGGGAACATCGTGTACAGACTTATTCCCATGACGATTCTCTACAA
TCAGAGAGATAACAGTGTAGCCAAACTCTTCAGCCATGTCAATGTATGGTGCTACCTCTC
GCTCCCGGGTACTAGTATTGGATACGATAACCGCATCTAGTCCTATCTCCATAGACCGTC
GCGCTTTGTCCATGCACTTTTTGTGAGATTCAACTACCCTCTCAGCTTTCCACACATACT
TGCCGAACTCCATTTGGTAATCGTCCGCTTCGCAGATAGACGCCTGCCGGCCTGACTCCC
AAGTTTTGTTATACAGAATCTCGGCAAGAGAGGACTTCCCGGAGCCCGGGAGTCCTCGAA
TGATATACATGATCTTTTCTTGCATACTTCCTCCTTTAAATTCGAGCCATATCGAAGTAG
GTTTCGATCAGAAGTCGTCGCTGTGCATCGGTCATTTCTGAGAACCGAGCCTCATCCACT
TCATTTACCAAGAAGTCTACCAGAGGATAGAACTCTTCGTCAAGCGTCTGCTTGAATCCA
GCCGGGTCTTGGTACATCGCATTGACCTTCTTACTACCCATCCTCATGATAAACTTCTTG
CCGAGGTAGTGAGTTGACTTGCACTTCACTGTCCCTAGCCCTGTTTGTACCATGTAACCT
TCTTTCTCACAAGAACTCAGAAGACCCTTTGCATGTTCCCAGCTACACTCTATGTGCTCT
GGACGAAGAATCTTTACAGAACTGTGGTCCGTTACACCTAGGGCGTGAGAGTCAAGAATA
TATTCTGAAAGCTGGCCCCCAGTGAAGATATCGCGGATACCGATCAAATATGCCCCTTCT
TCTTCCTCGACAATGTGAGGGTCGTCTGGAGAGCATATCTCGAAGTTATATGTGAAGCCT
GCGATGTAATCATCTGGGTTATTCGACACATACTTCTGACCAAGGGCAACGTAATCGCCG
GTCAATGACCCCGTAGTACTGAAGATAAGGGAGCCATTGTGCTTAGTCACGGAGAACATG
AATCCATTCACTTTCTCAATCAGCCGCACACGGAATTTAGGATGGAGGAAACTCCCAGCA
CCATTCTCTCCCAGATTAAACACCTTCTCGAAGGGGCGTACAATACAATCACCGGAGTGC
TGGTCAAATACATGACCCCGTGCCTCCAGAAGGAGCGGATGCTCATTCCACAGGTTCTCG
TAGAACACCTTCCGGGCATACTTTATAATCGACATACCGTTGAAGTGCTTAACAGTCACC
AGCTTATTAGCGATCAGGTCTTTGTACATTGCAATGTTGAGATTACCTTCCATCCTACTT
CTCCTTATTTCAATTCGACAATTGGGCAGAATTTAGCCTTCTTTGCCTTTAGATACGATA
CCAGAATCCCATCCTTGCTGGACGATTTACTCGACATAAAGCGAGAGAATCGGTAGAAAA
CCCACACGCCATAAAAGACGGCGATGATTCCAATCCATACAAACCCCATGACAGCCAGAG
TCCCACCTAGAACATATCCGGTAAGTGGACCGAGAGCCAGAGAATACAATACAGAGCCTA
CAACAGCAGCGAGCGCTACAACCGTTAACGCCAGCCACACGACGAGGAATACTGTCTGCC
AGAAATAAGAGCAGAGGGTTACAATTTGCCAACCGCTTGAGATAGCCGAATAGACCGGGG
TCTCGAACGTACGATGGAGCCGCATATGCCACGAATTCTTATCAATCTTCATGCTTGTTC
CTCCGGGAAGATGTTGAAGTTAAGTACGTTATGCTCTTCAAGTTCCATGGAGGCTACCAT
CTTATAACCTCCGAGCTTGTGAACGATCTCCAGTATACTCTCAATAGAGAATTTGTCAAC
ATGCTGCTTCATCAGATTGGAGATTCGGCTCTGATGGGTTCCACACCGCTCTGCCACTTC
CCGTTGAGTCAGCCCAGACTGAGTGATGTAGTGTGCAGCAACAACCATTACAGTTTTCTT
GAAATCCTTGACCGGATCATTCATGTTAGTCCTCCTTATTGCTCAGCGTTACTTTGCCAT
CGAATACTCGGAATTGAGCGATCTCAAACTGATCCGTCCAGCGGGCGTCCTCGAAAGCCA
CTCCTTCAAAAGTCCCGTCTTGTTTAACATCAGAAGTCATGAGGACGATAAAAGGAACTC
CGTCGTCATGGATCAGTTCCACCAGAGTGTTAGCTTTAGGTTTAACATCCTCCTTGTCAC
GCGAAATCAACACATTGACCATTCTTTCCTCCTTTTATTTATGGTGGTCTACAACCACAA
ACTCTTGCTCTCGCATATATTCCCTCACTTCGTCGTAAGAGAATACCTGATGATCTGGGT
GATTGTCAATACCTACATCAAGGCGCTTACCCTTGGAAGGCAGGCTGCCGTGAGTGTGGC
CGTGCAGGTGCCAAGCTCCATGATGGGCAGTATTCCACGTCTCGAAAGGATAGTGGCACA
TGACGATCTTAACTCCGTCAATTCGAATCTCCTTGTACTCACGAATGTCTTCTACGTGAG
AGAGATTGCTATCAGCGATCAACTTCCACAGACGAGAGTCGCAATGATTTCCCTTCAGGA
AGGTGATACGGCCATTCAACTGCTTGATGATATCCAGCACAGGCTGTAGCTTGTTCTTTC
CTGCAAATGCGAAGTCTCCCAGATGGTACACTTCGTCAAAAAGGCCTACCCGGGAGTTCC
ACCGAGAAATAAGCTCCTCGGTTTGCTCCTCAAAAGTCCACGGCCTGTTGCAATACTCGA
TCACATTCTTATGGAAGAAGTGTAGATCGGAAGAGAAAAATACAGACATTATGCCTCCAT
TTCAAATTCGTAAATTTTGTTAATCCTGCGGATGAGTCGCATAGCGGCCCCTCCCAGCAG
ATGTTCACGCTCCGGCTCCATACTAGCCATAGCCTTGAACTCTGATAACATATCCCGACG
CTCGTAGTGGAAATCTACTACCCAGTAATGGTCCGCACTAATCTTACCCTCTCGGTACAG
ATGCCCTTCGACACCCAGCCAGCGATCCACTGCATCTCTCTTCAGTAGCTCTTTCGGAGA
CCACTTGTCTACACCCCGCAACTTCTCGAAGAAAGAGTAAGGACGAGAATAGTTGTTGAT
GGCTTCCTCTACAGCCGACTCAGCCTGCAATTTAATGAACTCCCATCTTGCAAGAACTTC
TTTAATCTCCTCGGTGGTGACCGGGGTGTAGCTGTACTGTTCTTTATATTCATTCCACAC
TGTACTGTTCTCCTACATAGCCTTTGAATTTGTCGGAACGAGTTGTCTTCTTCCGTGCCT
TGTTTTCCAGAACTGATGCCTCCCTTTCGAGCATGCTCAGTTGGTTGCTATACTTCTTGA
TCCGCTTCGAGTTACCACTCATCACAGAATTTGTAAGATTTGCGATAGTATCGCAAATTT
TCACTTCTCTTGCAACCGGATGATTACGGCAAGCTTCGATATACTTACGGTAGCTCTCAG
CACGGTTCTTATCGAGAATGACCAAAGCCTCTACGATGTGAGGCATCACGCCCATGGACT
CCAAGTCTTCCTCTGTCAACTCTGTATCCTCCAGAACGTCGTGCAGGAAGCACACAGCGA
GAAGAGTGTCCATGTACGGAAGGTCGGAGAAAGCCTGACCCGGGTCCATATCTTTAGGAG
CGTTGCGGGCCACGCAGACTGTATGCACCTGATTCAGATGGTAAGCATACGGGAAGTTGG
TCTCTCCGTACCGCTGACCTTCGTGAGCCGAGAGTGCGATCTCAATAGCGATACCAAGCT
GAGTGTTCATATTAGTCTCCTTTGTAGAATGTGTGGTTGCCTATCTTGATTGTGCGCTTG
TATTTCCTAGCCCACCGAGGAAGCTTGCCATTATTAGCCCGTCTCACCTTCCTCTCGTTG
AGGTAGTGATCGGCTCCTCTCGTTGGATCGACAGTATACCCATATAGGGCGTTGTATGCA
ACATGTTTCGCTGCCTCCCACTTGTCAAGCTCCTTGACACCTTGCCGCATACTCCCATCG
CAGGTGTAGGAATACTGGCACTTGAAGTAGAGCACTCCGGCTACTGTGCTCGGCCACCTG
TCGTGCTTCATTCTGTTCACTACGGTATGAGCTACAGCAATCTGACCGATCTCCGGTTCT
CCCCGGGCCTCGAAGTATACAGCTTCAGCCAACAACCTGCAAGGCTTTCTGTACTCACAC
AAGGTGCGCTTGCTTTCTATGATTTGACCGGGACTGGCTTGGAATTCCACTACAGGCTCC
GTGCCGTATTTTAGACGAGAGTATTCCATGTCGAGGAACATCCCAATGAAGGCTAGAAGG
ATCGACAAAACGAGAGCTTCTAAGGTTTCCATAACACCTCCTTAGACAAAGAAGCCCGCC
TTGTGGGCGGGCTGTATATCACTCGGCTATTATACTTCAGCCCAAGTTGTAACGACGGGC
GTAGTCTTTGTTGGGGAACTCGACCATCATCGACTGAATGTGAATGTTGCCTTTACGATC
CTTCTGGATTTTGGCATTTTTCACAATCTTACTAGCCTGCGCCTTTGCCATAAGCTCTGG
ACTTACAACCAGATCGATGAATTCTGCACTCCGATACCCGGCACCAGCCGGGGCCCAGAA
GCCGTACTGGGTCACCGTCGATGTAGGTACAAGGTACAGGTTGTGCAGACGCACCGCAGT
GTTGAAACTCAGCTTACTGATGTGCTTCGCTCCCCGGGTTTCAGTTTCAAAACACCCAGT
CATGTAATTGAATTTGTAAGTGAGAGCATCCATGAACTGAATGACACTTCGAATCTGGAT
AGCAGAAGTATCCAAGAGCTGGAATTTCTCAGCAGATTCGATTGCAGTGGTCACATTGAC
AGACTTAGCGTTGAACATTCTATATCCTCCTTATTCTTCAGGGACAAATACACCGCCGAT
TACCCGGCCAGCGCGAATTAGTTTACCAGTTGGGAACTGAGCACGCAAGGTCTTTTTGAA
ACGACTTTGAATTTGCACAGTCAGCCACTTACCCCGGTAGTTCTCAAAATTAGTGATCTG
GTCTTTACGGGCTTCAAAGTCGCCATACGTCACCTCAAGCATCTCTTGAGGTACAGGGTC
GAACACCACCAGAACAGCGTTTCCATTCTTGTCAACCACGTAGTTCACGATCTGCATTTC
CATATCGAAGAACTCTTTGTACTTCTGGAGACCGCTGGAGCGCTTACCAGATTCGTACAA
GCCCTCTGGGCTGCGCCACATGATACCCTCATAACCGTTCTGACGGTACACATCATGAAG
ACGTTTCAGTTCTTCAAAGGTGGAGCAGAATACGTACTCAACAAAACGAATGCTCCCCAG
ACCACTGTGTAGTGCAGACTGGAACTGGAAGTCTACCAGACGCTCCTTAAATGTCAAGGC
ATCTTTTACGATATCGAAGACATGGAACTCCATCCGCTTACGAAGACCGTAAATAAAGGA
CGTGCTGTCCACTTCCTTCAGGAGACGATCATACTCCTCCTCTCCGTTCTCTAGAGGGTA
AGCTTCAAGAGCTTTCTCGGCCTTCATCCACTTATCGTAAGCATCCTCTCGCTTGACCGC
CGAGTTGATATCCTCCAGCACCTCACCGTGGACGTACAGTTCGCCGTCCCACACATCACC
CTCTTGCATGATCTCTTCGAGTTCTTTGATGATGTGCGGTACGTTGTAAGGCTTGCCCAT
ACGAGACGCTAGCGTCACCTTGCCGCCGACCTTCTTAGCCATGCATCGCAGACCGTCCAG
CTTAGGAGAACCATAGCCTGCTTTATACTTCGCAGGATCGAGCGGCTTGGCGAGCATAGC
CATTACCGGCAGATCGTTGAGTTCTTCCTTGGTTGCACGATAACCCTTGTCCAACTGCTT
GCTCACCTTGGACTGAGCCTCAGACACGGCCTGCTCCTCTACAGAGCGTTTCTGCTTGCC
CGCACGGATGTTGGTAACCTGAGAGGTCATGGCTCCATGTTCTTTGCCGTATTTCACGAC
GATATTGAAGCTACCGTCTTCCTCTCCGGCCACCTCTACAGTCCACTCCTTGAACCCGCC
TTTCTTGTCGAGTCCATACAGTCGTTGAAAATAACTCATTGATCCTCCTCGTATTTGATG
ACAAGAACATCATTCTCCCGTAGGTGCCTAAGACCTTCGTCGAGCCGATATTCGTGCTTA
TAGTACACTACTTTGATCCCAGCTTCAACCACCGAGATAGCACAATTTTTACAGCAAGAG
TGAGTGACGAACAGGGTAGCCCCTTGGGCACTCTCAGGGCTGCGTATAAGAGCCCTCAGT
GCGTTTTCTTCGGCATGCCTCACCTCTGCCTTGGTTACTCCCGTTTCGTCCTCCAGCGGC
CCGTTTAGGTGCTCTGGAAGGGCATTATACCCGCAACTGATGATACGGTTACGTTTTACT
AGAACTGCACCAACCTTGGAACGAGTCGAGTCGCTGCACTTAGCGAAGGCCTCAGCGGCC
TCCATGTAAGCTTTAATATGCTTGACCTTCACGACTAGCCTCCAATTAGAACCGCGATCT
CCTCGTAGTACCCCTCTTCAGGGCACGCGAATTCCATGTAGTACTCGTCAGTGTCTGTCA
AATAGACAACGCTAGGCGGGTAATAGCTCCAAGTCGGGTCTCCGTGTTCAATCTCCGCTA
CCTTCAAGTTGCCATACACAGTGCGAAGAGTCTGTAGAGCCTCGATCACATCGTCTATGC
GCATTACAGTGGGTTCAGTCGGTTCAGTCATATCTCCTCCTTAGTTTTCTACTTTACAAA
CGGCATCGCCCCGGCGAAGGAACACTTGCACTGATGGCTTTCCGCTCCACTTGTCCCACG
CTTCAGCTTTAACACTGACAGTCTTAGGATTGATTGCAACCACCTCGCCTACCAGCATAA
CCATTCCAGTTCCGCCTAGGCAGAATGCTACTGCGTCGCCGATCTCGATATCCCTTCCTG
CTACGTCCTTCATTTGTACTCTCCATCTGCACCCTTGATTAAAATAACGAAGTCAGAGGG
ATCATCATCGCTGCCGTAGGCCTTTGCCCGAAACTCCTGCTCGTATTTATTCACTTCCGA
TGGCTCGCTACTACCTTCAAGGGTACTGTATAGCTCTTGCATGAATTTCCCAAGGTCCGT
GGTCGCATGTGTTGCCGACAGGTAGCCAAGAAGCGCTACCAGCATATCAATCTCTTCGCT
CTCCAGCTCCAACTTGTAACCATCGTAATCGTATACTTTCATATTTCCTCCTATCGCTTC
TGTCCGCTTACGTCAAAGTACGATTTCCATTCTTCTCCGAATAGTTCTCTACCCTTATCA
TCGTAGACTTTATGAGCCTCTTCTGCTGTTTTATAGCAACCGAGGTAAATTCTCTTACCT
TCATACCTGATCCGGGCAACGTACCTGTTGTTTCTAAAAAAGATACCGGGATACCCGTTT
GTATTACGGGTAGACGCGGGTTTATTCTGCTGCTGCTCCGAGTCCGTCGCCCACCGGCAA
TTAGACTTGCAATACCCTTTTGTCGAGTCCTTTCTATCGAGAGTCTTTTTCTCGGGTCGT
TCTCCCATGTCCTCATAGAACCCCTCGAATGTCTTCCACTTAGGATCGATGGTAACATCC
TTATAATATTTGTAACTCTTGTGCTTTGGGTTCGTACATCTGTCGATCATTGTCCTCCAA
GAATTGTATGTCGGAGTTCCGTACATTCCATGGGAGGATAGTAGTTTAGACTGCATGCAC
CCGCAACTCTTATTATCTCCTACCTTGAGTGCTCCAGTTCTAATTTTCAAAATTGCCCCG
CAGATGCATTCGCATAGCCAAAGAACCTCTCTTGAATTACTCCTCGCTCCAGAGTCTCCG
ACTACCCTGAGATATGTGAATGTCTGCCCTGTCAGGTCTTTAACTGCCATCCTACCTCCT
TACTTGACGAGTGGCTACGCCGTAAGCTGGCTTGCTCCCCTCGCCCGTGTTGTGTTTCTT
CTGCGCTCGGCTGGCATTGACGGTGTAACGTCCTTTGCCATACTCTTCGTCTATCCACGC
TTGAGCCTTAGCTCGCTGAGACGTGTGGACAAAGATGTAGTAGCCAGATGCATCCTTGAT
GAACCAAGTGGCCGGGGGCTGGAAGTCCAGCGCCGAAAACTCTTCGAACGATACCACAGT
CACCTTCTGCTTTGCAACAGTGTATTGACCAAATACAGTCTCTTTGTTACGTAGTTTATC
TAGCATCTGAGTAAAGAGATCAGTCATTGCGATTCTCCTTTCTCGGCTGTAAACGAGTTC
AAGCTCAAAGAGTGATAGGTGTTAATTCCGTAGAAATGCTTGTTGCACAGAGCGTTCACT
GCTTCTTCGTACTGCTCTTCTTCCAGTTGCCACACAGTCCCACTAATTGCAGAGAGTATC
TGCTCCCTGCTCGGCTTGCTTCCGACCCACAGTCGTAGCCTCCAGTCTGGTTCATACTCC
TCTTCCCACACAAGCTCCCACACTTCCACTTCAATATTTTCAGACATTCTTACCTCCTTT
CCCTGTATCTACAGGAATCTTATATAGACACATATTTCTCTTTCCGCCCTGATACCTAAA
GACCTCGTCTTCTAGAAGGATTTTGGGTACTTCATCCAGCAGTACATCCTCTAGAACCCC
TGATGCCTTGATTACCGTCCGAAGCTGTGCCTCGGAGGGCGGATTTACAGACCACATGCC
GAGTCGTTCATCTGCCTCGTACGCCTCCTCCAGAATAAGCTCCCATACTTCGATCATCGG
TAAACCTCCTTCTTCCGGATATTGTAGATTACCCACTTCTCTTTTCCAGAGACTGGACAC
GTAACGTCCAATTCACACTCCGTGTTTGTCAGGAGGGAAGCCACAACATGGTCCAGTGTC
TCTGCATCCGGAGCAGGAAGTCCCGGGTAAGTCGAGATTACCTTACGGATTGTATCCGCA
TTAGTATTGCCCAAGACACCTATGAAGTGGAGATCAAAGTCCGCTGCCCAGTCATCCCAC
TCTTGTCCAAAGATAACCCAAACGTGATTCATACGCCCTCCTCAGAATTTACATAATACT
TCTTGACCGTCGATGATCTCGTATTTGAGGCCCTCTCGCTCAGTAAAAACGCGAAGTCTG
ATTTTCTTAACGCCCCGGTGCACACCCTTTTTGAAGGCATCGTTAACACAGTCTCTCAGT
GTTCTAAACGGCCCTGCAATATTACAGAACGAACCTCCGGAACTTGCCCACACCGACAAG
TAGCCTATCTGCCTGTCTCTGTAGACATAAACTCCGTTGCGGGTCATCCGCTGAAGCTTT
CTTTTGAGTCTGCTCATACACCTGCATCCCTTTGCATCTCTTCGAGACACTCTTCCGGCA
TCACAGAGAGTGCCAGCTCTTCCAGAGAGTGAATGTAGTCCTCCAAGTAGGTCCACTCCC
GGAGCACCTTCTGAAGGTACACACCCTCTTTGCTGTTGTCAATAGTCTGTCCCTTCAGTT
CCTTAATCAATTCAAACATCTTGTATACCCTCCTTTAGGCGTAAAAATGGCCGGTGACTA
CTGTCCCGGCCTGATAATACTACTCTGAAATTTCTTCCATTGCAAGGCGTTTCTTATCGA
AAAATTCCCCGAGTTTCGTATCCAGTTCGTCTCCTCCGATGTAGAGTTCTTCACCGGCTT
TGACTCGCTCCATTTCGCTCTCAGTTAGGAAGTCCTTATAAACATACTCTAGCCAACGGT
CGCACCATTCTGTGTTGAATGCTGCGTTTCTTAGGCTGTTTTTGTAGTCAGCGGGAACGA
CTCCGAAAGATGGAGCATGGATCATAAAGTATGTGAACTGATCGATCGACACGGAATCAC
AAGCAAGTGCAATTGCCGTCATTGCAGATGCACACTCACCTTCCAGCCGAGCATGAACCG
GGGCTTCACATTCCCTGATCGCACGAATGAGGACGTTTGCAGTCTCAACAGACCCGCCAC
AACCATTGATTGTGATGTGCACCTCGTCATTATGACCTGCGTTTTCAAGAGTCCACAGCT
CTTCAGTTAAGGTCTCTAGCTTATTGATGCTATAGGGAAGGACGATTGAGTATCTGCTAA
CAACGCTCTGTGTACAAGAAACAGGCATAACACCTCCTTTAATAAAAATGGGGCCATAGG
CCCCTTGGTTGTTACTCTTCGGTAGCAGTCTCTTGGCTGTGGTTTTGACCCAGCATAGCG
AAGAACGGTAGAAGCACTTCACTGAGTTCTGCACTGATGGCCTCAGCTACTTCAGCGCCT
GCACGAGATACATTAATGAACTCTTCGGTCTGAACGTACTTGTCCACCTTCACAAGTGTC
TTGAACTCTACACGCTCCAAGAATGCCTTGCCTGCGGCCTCTGCGAGAGGTTCGAGGTTT
ACAACCTCCAGCGCACGATCTAGAGCGCCTTGATCGATCTCCAGACCGTTTTGAGCTGCC
ACATGGTTGAGGGTAGCTTTTACGCTCTGTACAGCGTAGTTCTTGAAGAATTCACGTTGA
GCTTTGTTAAATTTCATTCACTTTTCTCCTTTGTAGTACGACGACGGCGGGTTTTAGGTT
TTTCCTCTTCAGAGGAAACTTCCTCTGTTTCAATCTCTTTCGTATCAGTCTCTGCCTCTG
CCTCTGCCTTCGCCTCCTCGGTGGTGACCCGGGTCATCTTTACGGTCTTCATGGCGAAGC
TCATATTCGCGAACTGAGGGTCTTCTTTGTCGATTCGGAAGCCTTGATCGAAAGCGTTCT
GCATGCTCATCAGAAGACTGAAAACATCCCATTCTTGCACAATCACTTCGCTTCCGTCAA
GCTCTTTTTCAATTTCATGTTGTACGCCGTCTTGGCGGAACATTTGGACTTGACAGAGGG
TTCCGTACACCGGGTAACCTTCAATCGAACGCATGACTCGGAAACCTTCTTCCATGTGAG
CGTGAACGGACCGGAGCCACACGATTGGGTTGTACTCGACGATATTAACAACTTGCATAT
TCTTTTTACTCCTTGCGGGTTAGTGTTTTACAATAATACCACGTTTCTCAGGAATGTCAA
CCCATTTTGTTCTGGTAAAGGCTTGACATAGAGGAAATCTGTGGTATTATCAATATTCAG
CGTGGGAGAGATATGTCTGAAAGCACTCACCGGCCCACGCCTAGCGCCTATGAAGAGGAA
TCCACAAGGGCAACCTGAACTCGAATGCTGTTAAAGGGTGGAGACGACGCGAAAGCGGAA
TGACAGCAATCCGATGCAGCCTACATGCTGCCCCTCACCGAACTCATCTGCATGAGTAAG
AGGAATTGGATAGAGCCAAGGCACGTCTTAGGCAGATTAAATTCTGTATGGCGAGCCTTG
GGACGACTCTATCCTTCCCTAGCCGAATCTATCTGCATGGGGTGTTCTCCTGATACACAT
ACACAAATACCCACCACAGATAATCCATCAATCAACCCATAAGGAATTTCTATGAGATGT
AAGTGCTGTGATAGCGAACTCTCGTTTGAGACTCCAGTCATCAATAAACATACTGGTAAG
GAAGACGATCTATGCTCCTCTTGCCGTAGGTTTGCTCTCAATCCTACACTGGATCATGAG
TATTTTGGTGGTTGGTATCCAAGCGAAGGAGTTACCGCTCCTCTCCCTACCGGGGACTAA
CATTGTATATAGAGTGCGCGTACTCTGACAATATGTATAAATAGTAGATTACGGTGCGAA
TAATTCTTTAAGGGCTTGACAACAGGAGATTTCGTGCTATAATCGGAGTATATGGGGCTT
ACAAAGCTTCAGAGGATTTAATATGACTCAAGAAAGAAACAAGGGCGGTCGCCCTACGAA
TGCCGAGCTAGAAGCCCGGGGTATCATTCAAACAGACCTAAAAGCAGGACTCAGACTTCT
AAAGAAGAGCTTTGCAGAGAACATTAGGTTCCTACAAGAGCAATCTGACAATCCTGAGGT
TTCTCTACAAATGAGAATCAAGCTCAAGAAAGAGCTTTCTGATATGTTCGTCGCTTACTA
CAAAGCTGACATTGCACTCAAAAAAGAGCTTGCAAAAGGAACAGAATCAGGAGATAATGA
GAACACTGAAGAGGACAAGGCTCCCGGAGTAGTTCTCGCGTTTTGATTAATTGCCGCTTC
GTTCAATGGTAGGACGCCAGACTTTGAATCTGGAGATGATGGTTCAATTCCATCAGTGGC
TACCAAATGTGGTAAAAGAACATTCACCCGACAAGGAATGTGTCTCGCTCAGAGGACAGC
GAGTAAGAAATGTCCTCCAGAATTAAGGGTCGGTGAGCGTAATTGGCAACCTACTGGATT
CCAAATCCTCGTATTCTCTGTTCGAATCGGAGTCGGCCCGCCAAATTATATGGAACTTGA
ATCCTTCTGGGACCAAGTTCGCAGCCGGAGAGCTAGGCTATGCTGCTAGCGACCGATGTT
CTTTAGAGACTCGGTATTCTCTCCAATTGGTGTTGACAGGAGAGTAAGGCTCCTGTATAG
TGACACAAATTGCTCGTATAGTGTAATGGATGCACAACGGTCTTCTAAGCCGTAAGGTCT
AGGTTCGAGTCCTAGTACGAGCGCCAAATTTAAGATGGACGGAAGCTAAAGTGGATCAGC
ATCTGGCTTTTAACCAGACTTATAGTGAGTTCGAGTCTCACCCGTCCAACCAAATTTAGT
GAGTAAATCAGATAGGTTCTGAGGCTCCCTGCTAAGGAGTACGTCTGCTAACGCGGATGT
GGATCGAGACCACTGCTCACTGCCAAAATCACTGCCAAAATAAGTGATTTTAAGTGTTGA
CAAACTGAATTTTTCATGTTTTAATACACTTATCAGATGAAGAAATACTTAGCGAGTAGC
CCTGAGTAGGTCGGGAACTAGTCTAGAAAACTAGGCCGCTGTGATGAACGGTGAGGGTTC
GACTCCTTTACTCGCTGCCAAATTAAGGCCCTGTGGTGGAATTGGTATACACATCAGTCT
TAGAAACTGACGCCGAGAGGATTGAGGGTTCAAGTCCCTCCGGGGCCACCAAATTAAGAA
TCTCTTTTGCTATTTGGTTAGGGTTCGAGTCCCTACCCGGTCACTTAAGGCCGGTCTATG
GCGGCCAAGTAGTGAGAGAGCTAGGGGACTGACGAAGGTAGCGCTGCTTTCAGACCTCCC
CGGCGCTATAAAAGTTATCATGAAGGGGGTTGACAAACAAGAAATTCATGATAATATAGG
TACATAGAGTAACAAACAATATAGCCGGATAGCTCAATTGGTAGAGCACCCGACCGATAA
TCGGATGGTTGAAGGTTCAAGTCCTTCTCTGGCTACCAGAATTTACAGGCCCATTAGCTC
AGTCTGGACTAGAGCAAGCCCCTGTCTAGGGCAAGGTCGTCGGTTCGAATCCGACATGGG
TCGCCAACAAGGTCGTTGTAGAGATAGATGGCTACTATCATCCGGCTGTAACCCGGACGC
CATAGGCTAGAGGTTCAATTCCTTCCTTCGACCACCAAATAGCGAGAGCGTTAAGACTGG
CTGGCGCTGTCACTCGGCTCATAACCGAACACAAGTGGTTCGATTCCACCTCTCGCTACC
AAATTGCACTCCGAGGAACCCTAAGTGGTGGGTTCAGTGTCGCTACCTATCTCGGTAAGG
GGAAGCGCCTAAGCTGGCACGGGTAATTGAGTAGCTGTGATAAGCTACTGGCAGCCAGTA
TAAATCGTCCGGCTCGGGCCCGTTATAGCTCCTAGATTTGAATTAGATGCTGTAAGCCAT
AGGAGGTGATCCAGTATCTCCCGGAGGGGCCCGGGTTAACAACCTCATGAATTTAGCCCG
CGTTGGCCGAGAGGTTTAGGCGGCGGATTGCAAATCCGTCTCACATCGGTTCAAATCCGA
TACGCGGCTCCAAGGTTTATATTGGGATGTAGCTCAGTTGGTAGAGCAGGGAGCTGTTAA
CTCTCAGGTCGCAGGTTCGAACCCTGCCGTCCCAGCCAATTTCCAAAAGAGACTCTTCGC
TTTAGAAGCAGAGTCGAACGCCCGATACGGGCCTTATTATCTCCTCGTAGCTCAGTCTGG
TTAGAGTGTCTGATTTGGAATCAGAAGGTCGAAGGTTCAAATCCTTCCGGGGTGACCAAT
TAGGAGTACATAGTGAAGATCAAATCGCGTGTATGTCCAGCGAAGACTGAGTATCATCGC
AAACGAAACAAATGTCCTTGCTGTACGCGGCCCAATTACGTAATGGAGTTGAGGCGTCGT
CAGGAACTCAAGGAAGCGAGACTTATGGTGTCTCTAGTGTAAAATAAAAGCGGGTATAGC
TCAGTTGGTAGAGCGTCTGCCTTCCAAGCAGTTCGTCGTCGGTTCGAGTCCGTCTATCCG
CTCCAAACAATGCATTTGAAGCTAACTAGGTAGAAGCGCTGGGTTGAAATTCCAGAGGAC
TTGGATCGTTACCAAGCAGATGCACCAAATAAAGCAGTTATAGATTAATTGGTTGAAATC
GCCAGACTTTCAATCTGGTGTTCCGGGTTCGATCCCCGGTAACTGCTCCAATCAAGTCCA
AGCCGGAACATTCCTCCGTATGTGCTTTCAGCCGGCTTGGCATTATTCGTGTGGAGATTG
TGTAGCCCGGATGCTGCCTTTCGGTTGTGACCCGATTGTTGGGGTTCGAGTCCCCGCCTC
CAACCAATGTCGTGCAATGGTATATCGCGTTGACGCTTTAGGCTCTCTGGATCGTAAGCC
AGTAAAGCGAGAATAGCGGGCTCTCGCAGCAGAGCGATAGGTGGTTCGATTCCACAACAT
GACACTCTTTCCTCCTCGCCTGTGCTGAACGGTTTCCTCCTCCTTTCCCGTCTCACAGGC
TCTATTCCCTGCCCTTTAAGCATTTATGGTGATGCACCGGCTTTGTAACCCGGCGAATTC
TGTTCAAGTCAGGAATGGGGCACCAATTCACTATCAAAGAACCCGCAAGCTCCCGATAGG
GTTCACACTCTATAAGGTTGTAGCATGGAAAACACTAAGGTTCCTGATAAAAAACTCGGA
CCAATCGCGCTAGCGATTAAAGAGGCCAGAGAGAATTTCGATCTCAGCCCTCATGTTATT
GGTCCAAAATCTTTCAAACAGAAAGAGTTCACAGACAGCGATGCAAAGATCACTGTATTC
GGTGGTGCTGCTGGTGCTGGTAAGTCTTATCTCGGTGTAATGGACTTCATGAAATATATC
CATGATCCTAACTTCCGAGGTGTAATGACTCGTCGTACTACTCCTCAGATTAAAGGCCCG
GGCGGTCTGCTTGAAAAAGCAACAGACCTATTCAAGCTCGTTGACCCTAAAGTCAAGTGG
AAAGACAAAGACGGCAAGTTTGTGTTCTCTTCTGGTGCAGTTATTTTCCTTCGACACTTC
GAACAAGAATCTGACAAAGACTCCTTCCAAGGTTGGGAGGTTAGTAAGTTCCTAGTCGAC
GAAGGTCAGCAGTTCACAGAAGGCATGGTGACATACCTTATCTCTCGTATGCGTAACCCG
AAATGCTCGGTTGTTCCGCACATGAAGATTACATGTAACCCTGATTACAATTCGTTCCTT
CGTAAATGGATTGAATGGTGGCTAGACCCTGACACAGGAATCCCGATCCCTGAGCGCTCC
GGCGTAACTCGCTGGTTTGTACGCAAAGGCGGTAAGATGTTCTGGGGAGACACCAAGGAA
GAGTGCATTGCCCTTCATGGTAATCCCAGTCTAGCGCCTGACGATGAAGATCAGGTGAAG
CCTATCAGCTTCAAATTCATCGCGGCTAACTGTTTCGATAACCCAATCTTGATGCTCAAC
GACCCTGAGTATGTAGCTAACCTTGAAGCCCTTCCTAGGGTAGAACGTGAAAAGCTCCTA
GACGGTAGCTGGCACGCTAGAGAAGAGGGAGCTGGATACTTCACAAGGGACTGGTGCCAA
ATCGTTCCCCACCCGCCTCTACGGGTGCTTAAAAGGGTACGTGCATGGGACATCAGCGGT
AGCATCGAATCCGAAACCAACAGAAACCCCGACTGGACAGCCGGCGTGCTGATGAGCCGT
ACAAAAGAAGGCTTGTTCACTGTCGAGGATGTTGTCAGAGAGCGTAGGTTGTTCGGAGGT
GTTTTCGACCTCATTCTAGAAACAGCCAAGCAAGACGGTCAAGACGTTGAAATTCAAATC
CCATGTGACCCGGGGGCTGCTGGTAAAGCTTATGCTGCCCAGCTAATTAGAGACCTAGCG
GATCATGGATTCCACGCCAGACCGAAGACAACTAACAAATCGAAAGTTACTCGATTCGCC
CCGTTCGTATCTGTAGCTGAAACTAAATCAGTGCAAGTCGTTGCGGGAGATTGGAACGAA
GATTACTTCGATGAACTTGAGCGATTCGACGGAAGTAAGGCTATCAAAGATGACCAAGTT
GACGCTACATCAGATGCATTTAATGCTCTATCCCTTGGCAGCTTGATTATCCCTGATTTC
CTACCGCCTGATATGACTCAATCCAACAAATTCAAACTAAATTAATAGGAGGCGGCGATG
GCCGAGACTGAAAAGACAGCACCGGGTATCCCGCGACTTCGCCTAGGCGAGATTGGTAGT
ACAGGCCTTAAACAAGTCAATGGTACTATTCTGGAAGAACGCCGACCAGAACTTCGGTTC
CCTAGGGCATGCAGAACATTCCAAATGATGGCAGAAGACCCTACCATCAAATCAGCTCTT
GATCTCTTCGAAATGATGATGAGCAGGGTTGACTGGGAAGTTGATCTAGGAGTTGACCCC
GACGAGGCTATGAAGGCCCGTGGTAAATTCCTGAAAGAGTGCATGCATGACATGGAGCAC
AGTTGGTACTCCTTCATCAAAGAGGTTACTAGCTTCTACACCTACGGATTCTCCGTACAT
GAAATGGTGTTTAAGCGACGTGAAGGATACCCTGTATCGAAGTACAATGACAACAAATTC
GGTATTAAAAAGTTGCCAATCCGCTCTCAGTCTACTATCACCAAATGGTTGTACTCTGAA
GACGGTAGAAACTTCCTAGGGTGTGAGCAAAGTCTTGCCAATGTTGTCAATGGCGACCGT
TACGTCAACCTAGGTAATAAGGACGGAACTGTAGAGATTCCAGCTAAGAAGATGCTTCTT
TTCCGGGTCGATGCTAAACGTGACAGTCCTGAAGGTAACAGCCCTCTCCGTGCGTGCTAT
AACGCTTGGAGATATCGAGTAGAGATTGAAGAGCAAGAGTCCGTTGGCGTAACACGCGAT
ATGAACGGTATGCCTACGCTCTATCTGCCTCCTCGATATATGTCCGAAGATGCTACAGAA
GCTGAGAGGGCAGTCTACGAATACTACAAGCGAGTAATTCGTAACATTCAAATGAATGAA
CAATCTGGACTTATCCTTCCTCAAGCGTTTGACCCAGAAAGCCGACAACCTCTGTTCAAA
TTCGAACTGACCTCTTCGCAGGGCAGTAAGATGTATGATACAGATGCCATCATTCGTCGC
TGGGACAACAAAATTCTGCAAGCCTTGTTTGCCGATATGCTGAAGATGGGTCAGGACCAA
GTTGGTTCTTACTCTCTAGCTGGTGCCAAGACTAACATCATGGCTATGGCGATTGAATCG
CGGCTCCGCGAAATCAAAGACGTACTAGATAATAAACTCATTCCTACGCTGTTCGCACTG
AACGGTGATTATAGTCCAGACTTGCCGAAGCTGCAATACGGTGAGCTAGATGAAATCGAT
CTTGAAGAGTTCTCGAAAGGGATTCAGAGAATCGGTTCTGTCGGTGGTCTTGAACGTGAC
CGTGAGGTTTACAACAAGATTCGCAAAGCTCTCAAGATTAAGCCTCGACCAGACGACGAG
CCTGTTGATGTGGATAATATCATGGGTGGTCAGTCTCAAGCAGGTAAAGCTGGAGTAGGT
AATGGTGCATCAACATCCGCAGCCGGTAGAGACAACGCCGCTGCAAATAACGCATAAGGA
GACAGAATGCTGACTCTTGAAAGTGTTATTGAAGCTCTCAGCAATGCTTTCGGGGCTCCC
GCCAAGGCGGAAGAAGCCCCGGCTGTAGAGGTCACAAAGTCCCTAGACAATGAAAAGAGG
ATGGCTTTGTTTGTCGTTCTTGAGCCCCAAGAAGGCGACTCCACGACTGACCTACACGCA
GACACCTACTCCGAAGAGGAAGTAGAGAAAGCTTGCATCAACTTCAATACTCATTGCAAC
GTAGCTAATATCTTCCACAAGATTGAAACACAGGAAGCTGAGATTGTCCAGTCCTTCATC
GCTCCATCCGCATTCACAACTGACGACGGAAGAGAGATCAAGAAAGGTACATGGCTCCAG
TGGTGGCACTTCCCTGAAGGCTCTGAAGTAGCCGATGCACTATGGGAAGGCGTTAAATCT
GGTGAATTCACAGGCGTCTCTATCGGTGCCAGAGCTACAGTCGAGGATATCGAATGACCG
TAAAAGCAAAACGTAAACTGACCGATATCAAGTTTGAACACGAAGGTGCTCATGTCGCTC
TGGTCAGTAAGCTCCAAGGTGGTGCAGCCAACGGGCACACTACTCTAATCACAAAAGCTA
CCAATGGCGTAACTGAAGAGCTTATCGATAAAGCTGCTGAAGTTACAGTCACAATGCAGT
TCCCTGAGTTCCTTCGTAAGTTCTTCGGGATGTATTATGAAGATGCCGAAGTTCTCTCTG
TTGCCATGGGATATGGCAGAACGGAGTATCCAGAACTAGATCACAAAGACTGGATCGACC
AGCGGGTCGAGTCTATGTCCATTCTTAAGTCGGTTTATAAATCGGCTGACCTGAATCAAG
CTCTCTCCGAGCTTACCCCTGAGCAGACCCTTTCCCTTAAACAGGATCAGGAACTGCTTG
AAAAGGCTCTGGAGGTAGTCTCCGAGCAATCCAATGAAAATCCATCCAAGGAGATTACCA
ACATGGAAACTATCCTGAAATCGGCCCACCTAGAAGCTCTAGCTGACGCTATTGCAGTTG
AAAAAGCTGCTGGTGTCGCTGCTGTTGCAGAAGTTCAGAAGCAACTAGACGCCGCTAATG
TAGAGCTTACAGCTCTTCGTGAAATTGTCAAGGCTGCTGAGGCAGAGAAAACCGCTGCTA
TCGCTAAAGCCCGTAAAGAGCAACTGGCTGAAGTCCTCCCGGCTGACCAAGTAGAAGATA
CTTTCAAGTCTCTGGAAGTTCTGGACGATCAAGCTTTTGCACAAACAGTAGGAACGCTCC
GCATTCTTAAAGCTGCTGCTGACCAAAATGACATGCTTGTCGAGAAAGGTGTATCGGGCG
CTGGGGAAGAAGACGACTCTGTTGTCGCCGGTGTTGCCGCTATCCTGAAAGCTCGCCACG
GCTCCAAGTAATATCTAGCCAATCTAGAGGAATTCCAAATGGCTCAAAACATTATCGCAA
AAGATCATCAGCGTCTTAGCAACTGGCTGAAAGAAGAACAAATGGGCCACCGTGGCCTGT
TCTACACTCGCGAAACTCTGCCTGTCGCTGACATTGATGTTAAAACAACTGGTTCTGTCC
TAGACAGCACTGGCAAGCTGGTAACTAAGGCTACTATCGCCGACGCTACCTACATCCTAA
TTACCGACCTGCACGACTATGCCAACGCTCAGATGAGCCACGCTGTCGTCCTTGCTCGCG
GTTTTGCCAAGATCGGCTCCAAGGCTGTTATCTTCGGTGCTGACGTTGACGACGCTGATA
AAGCTACCGTCTTTGAAGCTTTCAAAGCCAAGAACATCTTCGCCGTTGACCAGATCGAAG
GCGCATTCGAGAACGTTACCTTCGCGTAAGCGAGGGTCGTTCCAGTAAATAAATACTAGG
AGTAACTTTAAAAATGGCTAACACCCGTTCCTACCTAAACGATGGTCAGTTCTACATTGC
CGACCAGACTGAAAACCTGCTTATCATCCCGAACACTTGGACCCTCGTCGAGAACATGGG
CGTCTTCACAAGTGAAGGTGTGACTCAGAACACTGTCCAGTTCGAAGAGATCGAAACTCG
CTACGGTCTGGTTAAGGATGCCATCCGTGGAACTCGTCACCAAGTTGCTAGCGACCAACG
TCGTCAACTGCGTGCCTTCGCGATTCCGCACTTCAACCAAGACGACTACATCACTCCAGA
AGATATTCAAGGTAAGCGCGCTTTCGGTGCTGACCGCGAAGAAACTCTGAACGAGGTTCG
TGCTCGTAAGCTGGAAACCATCCGTCGCAACTGGGCTAACACCGCTGAAGTTGCTTCCGT
TAGCGCTATCGTAACAGGTAAGAGCTACGCCCCATCCGGAACCATCGAGTACGACTGGTA
CGACCTGATGGGTAAAACTCGTAAGGTTGTCGGCTTCGACCTGACCAACCCTACCGCTGA
CGTTATGGGCAAGACCGAAGAAATCTTCGTCCATATGCAAGACAACAGCCAAGACGGTCT
GATCCGTGGTGACTTCGTTGCCCTGTGCTCTCCTGAGTTCTTCACTGCCCTGATTAACCA
TCCGAGCATCAAAGAGTTCTACAAAGCTTACCAAGCTAGTCCTCAATACTGGCGTGAACG
TCTAACCGCTCGCGGTCTGGACCTTCGCTTCCGTGAGTTCTACTTCGGAAACATTCACTT
CATCGAATACCGTGGCGTTGACCCGTACGGCAACCGTCTGATCCCGGCTGGCGATGCTTA
CTTCATCCCAACCGACAGCGGTGACCTGTTCGCTCGTTACTTCGGACCGGGCAGCACTTT
CGATGACCTAGGAACTCTGGGTAAAGAACTGTACGCTACCGAGCGTATGGCTGAAGACCG
TCGTTCGATCCTGATCGAAACCGAGTCCAACTTCATCCACGTTCTGCGTCGTCCACAGAT
GATCGTTCGCGGCACTGTGAATGCGTAAAGACTTCAGGGAGGCTTCGGCCTCCCTTTCGT
CGTAGGAGGGTACCAAAAATGGCTTACACTGGTGATCCAGCAAACAACCCTATCGACCGT
CTACGAGAGATTGTAGGGGACGTGTGGGAGCCTCCGATGCTTTCCGACGAAACCTATCAA
TGGGTGTTGGACAAGAACGAAGGCAACGAAAGACGAGCCGCACTTGAACTTATGCGTATG
ATGCTCTTCCGCCTGACAAGGGGAATGAGAGAACGTACAGGTGATATCGAAGTATATGGT
GCTGAGTATTTCAACAACTACTTGAAAGCTCTTCAGCTCATTCTCAAAGACCCGAATATT
ACAATTTCTTTGGCCGTGCCATATGCTGGCGGTATCTCCAAATCGGATATGCTGGCTAAT
GACCTAGACCCCGACAACGTAACACGGGAATTCTACATCGGTTTTGCAAGACGGGAGAAG
CTTTACAACCAGTGCAACCCCGGTCCTCAAGACCTTAGCATGGGGTGTGATTATCTTGGC
AAGCTTCAGTTTTAAAACAGATCGCCGTCGCCTCACATCCTTGATTAAGAGGGTGGAGGC
TCTAGACGGAACAACGGTAGAAGTTGGTTTCTTCCCCGAGGACCGATATGGTTCTGAAAA
TGGAAACCTACCAGTTGCACAAGTTGCAGCTTACAACGAATTCGGTACTACTCGCAACCC
AACCCGGCCATTCATGGCTCCAACCTTTGAAGAATTCACATCTCAATTCCACTACGCAAG
ACTAATGAAGTCTACGTTTGAAAACGTTATCCGCGATGGAAGACAGGTTAATACTCTCCT
GAAGAAGCTGGGCAGAATGGTTGCAGAGCAGATGCAAGTTAACATTGACGACTACCCGGG
CTCTAACAGTCCCGCATGGGCAGCTTACAAAGGGTTCAACGATCCGTTGTTCCATACTGG
CAAGATGCTTGAGTCTGTCAAGTTCCAAATCCATCGGAGGCAATAATGCTCTATCCTACC
TTCTCCATGACCAACTTTGTCAAGCTAGATTTGATTAGAAGAGGGCAGCCCGGAGACGAC
GGTTTTGGAAGACCTACCCCTCCAGTTGAGACAGTAGTGACAATCACAGCTAACGTTCAA
CCTATCGAAAAATCGACAGACACTCGTATCCTTCCAGAGGCCGACAGAAGCAAGGCTTGC
TTCAAGGTGTACTCCAGAGGCGAAGAGATTCGCCAGTTAAAGGAAGGACCGGGAGGGTGG
AGTGCCGATAGATTCATGTGGGAAGGAGAGTTGTATGAAGTTATGAAAGTCATCAACTAT
TCGATGGGAATCCTAAACCATTACAAAGCAATTTGTATGAGAGTAGAGAGGAATAGTACA
GCATGATCCATACAGAGCTAGAAAACTCTCTCTATCTTATGATAAAAGAGTTGTTCCCTG
ATTGGCGTGTCATCCAAGCATACACAAATAATCAGGAACCGCAAACCCCTTATCTGGCTA
TCGATATTAAGAAGCTAGATGAACTGGGAAGGGAAAATGTCTCCAACATGAGCGATCCCA
TTAGTCCTTCGCATGGAACTGTTCAAGTTCAACAGGACTTCGCAGCCAAGGTCGTATTCG
AACTAGTTGGCAAGTATGGAGAGACAGCAAGTGTCTCTGACATGGCAATGGCGATTACTC
GGGCTCTGCGTACTCCAGCAGGACACGCCGCCCAAAGAAAGTTTAATCTATCCCTTTTCA
AACTCCCTAGCACAAGACGAGTTCCTATGCTAAGAGAAACAGATATGTACATGTTCTATC
AAGTTACATGTGAATTTGGATTCTCTGTGATAGAAACCACAACTCAAGAGTTTGCTGCTG
GGGCTGATATTCACGGCGTTTATTACGACGCTGGTCGCCCGGGTCACATTATTGAGTCCC
ACATCGACATAAACTTCGAACACTAATAGGAGACAGGAATGACTGTTCTTACTGATGTAA
TTGACATTCAGATCAGCCGTGAGACTGCTGCTGTCGCACAGACAAACTTCAACGTTCCTC
TGTTCATCGCTTCTCACACAAACTTCTCGGAACGTGCCAGAGTCTACAACAGCCTGAAAG
GTGTCGCTGAAGACTTCGGAGAATCCGATCCAACATATCTTGCCGCTGTTCGCTACTTCG
GTCAAGCTCTCAAGCCACGCAGTCTAGTTATCGGACGCAGACAAGTTCCTTCTGCTACCG
TCTCTGTCAGTGTTGTTCAGGAAGGACAAAGCTACGTCCTTACCGTTAACGGACTACCTG
TCAGCTATGTTAGCCAGCAAGACGACACTGCTACTCTGATTGCCACAGGCCTAAAGGCAG
CTTATGACGTAACTCCGGTAGCCGGCGTAACCGTCACAGACAACGAAGACGGAACTCTAA
CCGTCGCTTCTAACGAAGACTGGAGCCTGAAGGTTTCTTCTAACCTGACTATGGCTGCTG
CTCCCTCTACTGAAGGTTGGCCGGCTGCTATCACAGCAGTTCAAGGAGAAAACGACGAAT
GGTATGCTCTGAGCATTGACAGCCACGCCGATGATGATATCATGGCAGTAGCTACTCATA
TCGAGGGCACCAAGAAAGTGTTTATCGGAGCCACTGCTCAGGCTAACACCAAGACTTCCG
CAGAGAATGACATTGCTTCTCGTCTGGTTGCTGCCGGGTTCCAACGTACGGCTCTAATTT
ACCATCCAAACGCTGACGCTCAATTCCCTGAGTGTGCTTGGGTAGGGTACCAACTGCAAG
AGCAACCGGGTAGCAACACATGGACCCATAAAGCTCTGGCCGCTGTAGACGCCTACCGTC
TGACTCCAACCGAGTCTACCAACCTGAAGAACAAGAAAGTTACCACGTTCGAGCGTGTTG
GTGGTGTCAACCGTACATTCGGTGGGGCTATGGCCGGTGGTGAATGGATTGACGTAATGA
TCTTCGTAGATTGGCTGGAAGCCCGCATGACCGAGCGTCTGTGGTTCCGCATGGCCAACT
CCAAGAAAATCCCTTACGACTCTGTTGGTGCAACAATTCTGGAATCGGAAATCAGAGCCC
AGCTTAACGAAGGAATTCGTGTTGGTGGTCTGGCTGAAGCTCCAGCTCCTAAAGTCTTCG
TACCTGATGTTCTATCCATGTCTCCGAACATGAGAGCACAGCGTATCTTCGAAGGCATCG
AGTTTGAAGCCCGTCTAGCAGGCGCGATTCACTTTGTACACATCCGAGGGACCGTGACTG
TATAAGTCTGGTCCTTCCCATAAGGAGACTCATAAATGGCTGTTCAACGTCTAGCTACAT
TCTCTCCAGCCGATGTTACCATTGTCATCACTCACCCTGCAACTGGTGAGAGCATGGTTC
TCGGCGGATTCAGCGAAGACAGTATCGTTAACATTGAAAGAAACGCTGACACTTACGTGA
TGTATACCGGAGCCGACAACACATCGACTCGCGTCTACAACGCCTCTAAGTCTGCTACTC
TAACTGTTAGCCTTCAACAAACTTCGCCTTCGAACGACTTCCTGACTGCTCTTTACAACT
ACGACGACGCCCGTCGTTCGTCTGAGGGATTGTTCACAATCCACGTTAAAGACAACTCTG
GTCGTTCCGACTACTTCAGTGATGACGCCTATGTCGGTGTTGTTCCGGGTTCTAACTTCT
CGAACAGCATGCAGACACGCGATTGGGTTATTCATGCACACAACCTGCAAACTCTAATCG
GCGGTAACGCCAAGCTGAGCCCGGGTGATGCGGATACTCTTCGTAACCTAGGTGTCACAC
TAGACCAGCGCTGGCTGTAATATAGCGCTTATGCCACATCGACGGGGAGCTGCCTTAACG
GGTGGCTCCCTTTGTTGTTTCTGGAGGATAGAAATTGATTACCACCTACTCGCCGCGTGA
CGTGGTTGTAACTCTCGCTGGTATCCATTCCGTGACAGGGTACGCAGAGGGAGAGTTTAT
CAGAATTGTAAAAGATATTAAGCCTTTCATCAAGCACGGTTCGATGGATGGTGAAATCGC
AAGGGTCTACAACAAAGACCAAGGCTGGCGAGTAGAGTTGACAATCATGCAATCCTCGCC
GACCAATGATATCCTTTCGATGCTTTACAACGTGGACATTGCAACCCGAATGGGAAAATT
CCCACTGATGATTAAGGACACGAAGGGTTCTACAAGCTTCCTAGCTCTGACAGCATGGGT
GGAAGACCTACCTAGGGTATCTTTCTCTGGACAACTGGAGACCCGCACTTGGATTCTAGG
GTGCTCTGAGGTCGCCATGAACATTGGCGGAAACGTCGATCAAAGTTTGGTCGAGCAAGC
TATCCTGCTGGGAAGCTCTCTTCTGCCAGCCCTTACGCAATTCGGAGGCTTCTAATGGCT
AACACGGTATTGACATATAGCCCATCTGATGTTAAAATTGTCCTTTGTGGGTATGCTCTC
ACTGGTGTAGTTTCTTTCGAAATGTCTTGGCTTTCGAGACCCTACACAATGGTTCGAGGA
ATCAGAGGGCACCACACCCGGGTCTTCAATAGAGACCTTTCTGCGCAGATTAGAATCGAG
GTTCTTCAGACTTCGGTGTCTAATGATGCATTATTCTCGCTAGTAGAGCAAGACCGAAGA
ACACAATCCGCCCGAATCACCTTGTCTGTCAAGGATACCCACGGTAGCACCATGATGAGC
ACAGACAACGCTTATGTCAATGGCTACCCTAGCATAACATTCACCGATGGAATCGAGAAC
AGGGTCTGGACTATTGATGTTCTGGACTGGACCGATGGCACGGTAGGCGGAAACCAGCAA
GTCGGCTTCGACGTGTTTGGAACTGTTCAAGGCGCTCTTAGCTACCTCAGATAAATAAGG
AGTATTTATGTTCGGAAACCAACGTACTGTCATCCACAATGGGACAGAATACACTATCAA
CCTTCTGCCAGCAACTCGCGGTATCAGCGTACTGAAGCAGCTAACCAGCTTGCTGGGTCC
ATCCCTTGCCAAGTATCAAGCTGACCACGACTTCTCTGGAGCCATGAAAGAACTGTTCGA
TAACATCGACAAGGTTCCTGTTGAGAATTTGATCCAGAATCTGATGCTGACAGTTTTCAA
AGGTTCGATGGCAATCAATTTCGACACCGAATTCGCTGGCAAGTATGACCTGCTGTACGC
ACTCACAAAAGACGTGATCGAGTTCAACTTCGGCTCGGTTTTTTCTCTAATCGGTTTCGC
AGAGGCACCGACGGTATAAAGACCGGGGATGGGCAGTCCGAGACTTCAGTCACTACTGAT
CGAGTTCAGAGTGATCGGAGGGTTAGGGAGGCCCACCAGAAGTTCTCGCAGCATTGGGAG
ATTTATGTGGCTATCACTCACCCTCTCAAGCTAACGACGTATGGGGAACTGAGTACAGTC
CTCACTACAGAAGATTTGTACAACATCCTTGAGGTTATAGACGTTCACGATACTATCACT
CAAGTGGCAAAAGAACACGCAGAGGCTGATAAGAAATGATTACAGAAAGGATTGCCCAGC
TAACTGGGGAACTTAAATTTACAGTCGATTCCCGGCCTCTCACTGCTTTTGACAAGAAGC
TCGCTGGGGTCGAAGCCCGGCTAAGAGAATTCTCCAAGCTGACGAACAAGCGGTTTGGAG
TTAAGCTAACTCTAGATACAAAAACTCTGAGAGAGGAGCTTGCCAAGGCAGCCACTCAAA
GGGTAGTCTTGAAGAACATCGCGGTAGATGCTGCCGCTGTTCGTCTTATCTCTGAGAAGC
TTCAAGAAAGACTGAATGCAACGCCGATCAGGTTGAAGTCTGTTAGGCTAGACCTTAGCG
GTATCCGAGATCAGAAAAACTTCGTAAAGACGGCTCTCGGCCAGACCAAGGTCGATCTAC
CGGTTGAACTGGGCTTGGCTCAGGCTAGCCGTACGCTGTATGAATGGAAGAAGCGCACAG
AGAGTCGTTTTAAAATTCATCTGAATGCAGACATTAGCCGCTCTAAATTGCTCCAGAATG
CACGAAACACACTAAGGGATGTGCAGGGTAGGCTAAACGGTCTTGCTGTCGCTACGCCTC
AAATTAGGCTGTCTGTAGACCGTGCCCATCTTCGTAGAGAGATTCAAGATGTTCTTGAGC
AAATCAGACGCGAAGTGAGAATCCGCATTGACCTAGAGTCTTCCATCCGAGGAGGTGGTG
CAGGTGGATCGAGAGGTACTTCCGGCCATATCCGTCAAGGTATGGGAATGGGGATTGGTA
GCGAGCTAGCCGGGTGGGGACGCGGGTTTATTCCCGGCCTTGGTGGTGCGTTCGCTATTA
TGCAGCTTAACCGTGCAAACCAAGAACTTCAAGGTCAGCGCCTAGCGATGCAAGCTGTCG
GAGGCGGAGTACAAGGTGGCCAAGAACTGCAAGCTACCCTCCGTGATATCTCTCAACGCC
TAGGTCTCGACGACAGAGCCCTTGGTAGCTCCTATGTTAAGATGATGGCTGCTGGACAGG
CATCGGATTTCAGCAAGGAACAAGTTGACAGCATCTTCCAGTCTATGGCTGAATATGGTC
GAGTGATGGGCCTTGACGGCGAAGCGATGAAAGGCTCTTTCCGAGCGGTTGAGCAGATGA
TGGGTAAAGGGCAGATTATGTCCGAAGAATTGAAAGGTCAGTTGGCCGAAAGATTCCCCG
CTGCTGTAGCTCTGATGGCCAAGTCTCAAGACATGACTATCGCGGAACTGATGAAAACGA
TGGAAGACGGGAAGCTTAAAAGTGATGCCCTGATTCCATTTGCCAGAACTCTCGCTGAAG
AAGCCCGTAAAGGTGGCGCTTTGGATGCTGCTATGCAAGGCACCGCAGCTCAGCAAGGTC
GATTCCAGTTCGGATGGAACCGCACTATCGAAGCATTCGCCGCTGGCGGATTCGATAGAG
GTATGAGTGACTTCTTTAAGATTGCTGCCCAAGGCATGAAAGAAGCGCTTCCTCTGGTAA
CTGCACTTGGTGGCGGGTTCGAAGCTCTTATGAGGCCTGTCAACGCTCTGGTCGGTATTG
GAGGGGAGCTTGGTTCCCAGTGGGAGAACATTGCCAAGCAGTTCAACATGACCGGCACAG
GGCTGACTCTTACCACGGCTCAAACCCTAGCCTTGCTGACCCCTATGGGCAGGTTGATCT
CTGCTATTAGCTGGGGTGCGTTGGCACTGGAAGACTTCATTGTCTTCTTGGAAGGCGGTG
ATTCGGTGTTCGGGGACTTCTTGAACAACAACGTACAAGCTGCCGAGACATTCGAAAAGC
TAGCCAGTGAATCCAGTGAACTGAAGAACAATCTCGACGGTATCTTCTCTGTTGTCCCGG
GGCTAGCTGAAGCGCTCAAAGGTCTAGAGTTCAATGAAATGCTGGTCAGCACGATGAGAG
AACTTGCTGCCATCATGGAGTTCTTCAACAGTGTTGTAGAGAGATTCGCAATAGCCGGCA
AGTACAGGGACGCTAAGATTGCTGAAGCCGGAGGAGACAGAAGTACTATAATGTCGAACA
TCGACACGATGTACGCTATGTTCAATCCGGAAGATGCAAAAGACAAGGCCACCTTCATCG
GTGACAGGCTAGTAGCTCAGAACATTGACGCGATCCAGACAGAGACTTATACCCGTGCAA
CCCAGTCCTTGACTCCAGACCAGTTTGGGTATATGATGCAACGTGGTCAAGTTAGAAACG
AAATGGAGGGTGCCCTCAAGAAGAGTGCCATTGACATTAGTTTCAACCTCAATGTTTCTG
GTATCGATGCTCAGGGTAACGTCATGACAACTGAAGCGCAAGAGCGAGTACGAGAGATCG
TTAGTAACGTTCTTGAGGAAGAAATCTCCAGAGCTTCGGCAAGTTATAAGGAATCCCAAT
GACAATTGCTATCAAAAGAGAGAACGGAGACCTAATCTGGTTCGACGCTGTTACAGAGTT
CGGAAGACAGTACCGTGGCTCTGTGAGTTCCAACCCAATCGAAACGGGAGGAAAAATCAC
AGACCACATTACTACAGAAAACCCAGTCTTCACTCTTACAGCCGTCGTATCAGACGCAGA
CTTCAACTTGAACAGGCCCGTTATCAACGACAACGAGGCCCAGACATACAAGATCAACAA
CAAAGAGTTCGTGAATACGCAGCCGGTCAGTGTGCCGGCTGTTATCACAACTTCTAGATT
TGATCCGAGCCGAATTTTCCCGGAAGTTATTACTCAGTTTATTCCTCCTGAGATTCCTTC
TGCAACTGTCCTCCCTCAAAAGGGTGATAAAGTCGCTTATGATATTGAGCGCCAACTGAT
CGACATGCAGCGTAATGCTGAGGTCTTCAGTTTGCTGGACTTCCGAGAAGGTATCATCTG
GGATCAAATCGAAAGATGCATCTTCACCGACTTGTCTTTCACTGAAAACGCCGAGACAGG
TAGCAGCCTTCAACCTCGTATGACAATCGAGGCCGTGACATTCACAGACACTCGCTACAT
TGAAGTTAGGGTGAACAAGGGAAGAAAGACAGCCAAGAAGGAAAAGAGAGACACCAAAGA
TGGTGATACAGGGGCTAGCAACGCTACTGGACAAGACTCTCCCACATTCAAGCGTAGCCA
GATGAAAGAGGCTCAAATTAGAACTCAAGTTGCGAGGTGATTAATGAGCACGACTTACAT
CGACACACTTCCTTTGTACCAAGATAGGAAGTATAGATACGCGGTAGCTCTGGAGGGCAT
TTCCCGTGTCCTCCAATTCTACTGGAACAGCCGCTCTAGGCAGTGGCACATGGACATATT
CGACGAGGAGCTTAATCCAATCCTAACAGGGTTGGCATTGGTTCCACAATACCCGATAAT
TGCAGATTACGCTATGCAGCACATCGGGTTTAATGGGTACTTCTTGCTCATGCCTGTCAA
CCTAGAACAGGTGAAGTACAAGCACGATGCAAGCGACATTGTACCTCAGTTTTTTGAACT
TCTGTATGTTAATGTTGATTTGGAGGAAGATGCAGAGTGAGACAATACGACAGAGTATAT
AAATTGACGCTCGGTAATACTGAGTCTGGTCAGGGAGTAGAGATTACAAACCTTCATCCT
GACGGAAGTGTTAACAGAGAAGGACTTCAGTTCAGGTTCGATATCTCTAAAAGCTCCGAT
AACAAAAAGAGCGGCAACTCGGCTACTGTTGAGATATACAATCTGTCGATTGCAACTCTA
AACCTTCTTGAAACCGAATACCTTACTTGCAGACTAGAAGTGGGGTATAAAGAAGCTGGA
ACTTCCGTTGTTCTAGATGGTAATGTTGTCGAAACATCCACAAGAAAGAGTGGAAACGAC
TACGTGACTCAGCTTATTCTTGGTGAGGGTTACACTGCTCTAACTGAGACTAAGCTGAAG
GGCACAGTATCTCCGGGCAAAACTGTAAAGGATGTTATCGAAGAGATTCGTCTTCAAATG
CCGGGAGTGGACAGAGGAGCTTATACAGGCCTCAATTGCAATAACCCGATCATGTACGGC
TATCAACTCCGTGGTCTGGCCAAAGACGCTCTAAACAGCGTGTGTGAAGCCAACAATATT
GAATGGAACATCAGTGGAAACGTTCTAAACGTCACAGACGTTAACGGACCAACAACGAAG
AGTGTTCAAATGGCTCCTCTCGTAAACAGAGAGACCGGTCTGATCGATATCCCATTCTAC
GCCAGTGCCAGCGGAACGGCACAGAAGAAAGATAAGCGGAGACGGAGAGGCGTTCAATTC
AAATGCCTTCTCAATCCTGAGCTAACTCCCGGGGTTCTTGTCCGGGTAGAGTCGGATAGA
TTGTCAGGAACTTTCAGAATCAACAACGTCCGAATCTCGGGCGGATACAGAGACAACGAA
TGGTACACTGAATGTTGGTGTTCCGATCTTAACCAAGAGGATATTGACCAATGAGGAGAA
CAGGCCTACAAGAGCTTCTGAACCTGCACTCGTCCACGGAAGGCTCGAAACAGTATACTG
CCATACCTTGTGTGGTATTGCGCGTTCTGGACGATTTTAGACGCCTTTCTGTCGATGTTA
GACCAGTGGTCAATGACCTGTACAAAGACGGCACATCAGAAGAGCAACCAGAGATTCTAT
CTGTCCCTGTCATTATGCCGGGAACAGCTAACACCCTTATCAGCTTCCCATTGAATGTCG
GGGATACGGTGTTGTGTGTATTCTCTCAAAGAACTATGGACGTGTTTAAAGGCTCTGCCA
CTGGACAGCCTCACACTCCAAACGATCTTCGTAAGTTCAACATGGCGGATGCCATTGCCA
TTCCCGGGCTGTTCACCTTCCCTAGGTCGATGAACGACCCTGCAAGACATAGTTGGCCAC
ATGATACCAAAGACCTTACTATTGCCCACAACCTGATGACGGGACAAGAGGCGGAAGTTA
GGATCAAAGCCAATGGAGATATTCTAATCAACAGCCGCAAGACTATAAGCATTAATGCTC
AAAATGTCAACGTCAAGTGTGCTTCACTCACTGTCGATGCAGCCCATACAACATGGAACG
GGAACATTGCACATACAGGTAACTACGTGCAGACTGGTGGAACTTCTACATTCAACGGTA
TTCCATTCCATACCCACAAACACGGTGGTGTTATGCCGGGCGGTGGTGTTACTGCTGTCC
CACAAGCTTAAAAGGAGATCATATGGACTTGTTTGTCAACCCCGACACACATGACTTGGT
TTTTATAAATGGTGAAGCTCCAGTCACTCAACGAATGGTTGATATTGTTGCTCAGCGACT
AAAGATCAAACTGTACACATTCCTAGGTGAGTGGTTCTTGGATGATAGGATCGGCATCCC
CTACTTCGAGAGGATTCTTGGTAAGTCTAGGAGCCTTCCCGCTGTTGACGCGATATTCCA
ATCTGAGATTATGAGAGACCCCGGCGTCCTTGAGATTACATCTTGGCAGTCGGGGATCGA
CCCTCACACACGTGAATACTCCATGGAGTTCACAGTCAGAACCACAGACAATACAGAATC
CCTTCCCATCACTTTCAGAATGATAGGAGTTTAATATGGCTGGAATTACAGCAGAAGGTC
TAACGATTAAAGACTTGGACCAAATCCTGACAGACTACAGAAACGTAGCCTCTCAGGTCT
TCGCCGACCTAGTTTCAACTGGAGACGAAGTTGACACGTCTGGTAACTCTGCTCTAGGCC
GTCTGATCGGCGTAGTTGCTCCATCGGACGAAGCTATCTGGGAAGTTATCCAGATGGTGT
ATAACTCCTTCAACCCGGCAGCCGCAACTGGCGTGGCTCTGGACAACCTAGTGTCCTTCT
CGGCAATTAGCCGGCATGCTGCCCGTCCGACCCGGGCTCAGGTTGTTCTAGAGGGCAACA
TCGACACCGTTATTAACTCGCCTCCCGCTAAGATGATTTCCAGCAGTACAGGCCGAGTGT
TCCACCTGCTACAAGGCGTGATCCTCACTCCTAAAGCTTGCTCTGGTGTTGGAATCTTCC
CTCAGACAGTAGGCAACGACCTGACATACGAGCTTAAGATGTACGTTGATGACATTAACA
CAACGTCTATCAAGTACACAAGCCCTACCACTGGTACAGTAACATCCGAGTCCATCCTAG
CAGGACTGGCCGCAGACGTAGCTACAAATCACGGAAATACCTTGACAAGCTACGAACAGA
ATGGTATACTTTTCATAGTACCTATTGACCCATTCAACACCAAGACTTTCGAAGAAGCCT
CCAACCTGAGTATTCAGAAGGTTAGAAAGCTTGGTATCGCTGTTGACGACGTTATTGGCC
CTGTGCCTCAGCAAGCTCTAGCTATCGATACGATCTCCATTCCTATTGCCGGATGGGACA
GCGTTATCAACCCTGTTCCAGCTATCACTGGCCGTCTGCGCGAGACCGACGATGAGCTTC
GCGAGAGATTCCGAAACTCTAAATTCGTACAAGCCACCAACATTCTGGAGTCCCTAATCG
ACGGTCTGATGAACGTAGAAGGGGTAGAGGATGTTAGGATTATTGAAAACGATACCGACA
ACCCAGACCCCGTTCATGGAGTCCCGGGACACTCGTTCCTTCCAATCGTTCTTGGTGGTA
TTCCTACAGAGGTTGCACAGTCCATCTGGCTGAACAAGCCATTCGGGATTGGTTCTGTCG
GAGATACAGAGGTTCAGGTAGTAGACAGTCTAGGCTACACTCACAGAGTCAACTACCAGC
GACCTGTCGAGGTTCCAATCGAGATCAAGATCAGTGTAACAAACACAGGATCGATGCCAG
AGAACATTGAAGATATCCTACGGCCAAGGATTGTAGCGTACGGGACCGAGAATTACAAGA
TCGGAGACGACGTAATTTATTCTCGATTCTATGCTCCAATCATGGAAGTCCCGGGCTTCC
AAGTTAACAGCCTGACGATTGCCAAGAAAGGACAGACTCAAGGCATGGCGAACATCGAGA
TCGGGTTCAAAGAAGTGGCTACATTCGCCGCAGCAGATATCACCGTAACTACGGTGTAAT
GATAGAAGGAGAATTCCATGGCTGTCAACCAATTTGACAGAGAAGATTATCTGGAGGTGG
CCCGGGAACGGGTCACTGAACAGTTTAAAGAGAAGCCGATCTTTGATCGCTTCCTGCAAG
TGCTATTGTCTGGTAAGTTTGATATCCAGAATGCACTGGAAGACCTCCAGACTCTCCGGT
CTCTGGACACAGCCACCGGGAAGCAACTGGACATTATCGGAGACATTGTAGGGCGACCAC
GCGGTCTAGTGTACCAAGATATTTTCAACTATTTTGGATTCGCTGGAACGGAGCGTGCAG
GTTCTTTCGGAAGCCTGTCGGACCCTACGGTCGGTGCTCCATGGTACTCGGTCGGTGCTC
CA

In another aspect, the invention provides an OMKO1 bacteriophage targeting Pseudomonas aeruginosa. In certain embodiments OMKO1 binds a molecule of an efflux pump and/or Type IV pilus. In certain embodiments, the OMKO1 bacteriophage has a genome comprising a nucleic acid sequence comprising SEQ ID NO: 2.

OMKO1
(SEQ ID NO: 2)
AATTATTAACGAACGACTGGAGTCCAGCGGAAGTAGATGGTGCGTATATGCGCTATCGTG
GTTATGAGCGCATGGCAGGGACTATTTTTAATGAACTCAATAGAGCGGTACGTGTATTTA
ATATGCGTAATGGTGGATCAGTACAAACTGTCGAGTTAGATCCGCACGTGATCTGGCGCA
AGATTGTACAAGATCCGACAGTAGCCACTATTGAAGATTCTAACCCCATTGCAAATATCC
GTGAACAAGAAGCGATGACTTATCGTGGAGATGGTGGACGCGGTTCAACTTCAATGGTTG
CACGTACACGTATTTACGGTGAAGCGGATGTTGGTGTGGTTAGTGAATCAACTGTTGACT
CAGGTGACGTTGGCGTTATTGCTTACTTAGTACCTGATGCTAACTTTGTAAACATGCGTG
GTGTTACGCGTATGTTTGATCCAAAGACAGATGGACCAGCTAGACTACTTTCAACTAGTG
CACTGCTTGGTGTTGCAACCGAGCATGATGACCCTAAGCGAATTAACTTTATCTCAATTC
AGCAGCAGCAAGGTATTTATGCTGATGGGTATACAGTTACACCAGTTCGTACAGGCTATG
AACAAATCATAGCCCAAAGAACTAGTTCTATCTTTGCTGTAGCAGCAGAACAAGATGGTG
AAGTTGTAGCTATTGATGAATATGGTATCCAAGTTAAATATGCTGATGGTAATGTATTTG
GATATCAATTAGGTACAGTTCATGGCACAGCAGCTGGTGTTAACTACCCACACATTCTTG
TTACCTCATTAAAGAAAGGTGATAAGTTTAAACGTGGTGATACTATCACATATAACAAAC
GTTATTTCTCTGAAGATAGGTATACGCCTGGGCAAGTATTATGGAAAGCCGGTTGTATGG
CTGTTGTAGCATTTGATGATAACCTTGACACTTTAGAAGACGGTTCAGTTATTTCTGAAG
ACCTAGCTAAAAGGTTAAATACTCAAACAACTGCAATCAAGAATATCGATGTTAGGTTTG
ATCAGACTATTCGAGATATGGTGAAAGTTGGGGATCATGTCGACTTAAATTCAATTCTAT
GTATAATTGAAGATCCTGAAACAGCTGCTCACTCACTATATGACGAGGCATCTATTGAGA
CATTACGCAAACTATCAGCATATTCGCCACGTGCTAAGCTCGTAGGTACAGTATCAAAGA
TTGAATGCTTCTATCATGGTGAAATAGACGATATGACACCAAGTTTACAAGCATTAGCTA
ATACGTCAGATAAACAACGTGCTGAACGAGCTAAATCATTGAAAGAACCTACCTTCACTG
GACAAGTAGATGCTAACTATCGAGTGAAAGGACGAGCCTTAGAATATGACCACATGATTA
TTCGAGTGTATATCGATCATGATATCCCATGTGGTGTTGGAGATAAAGGCGTAGTAGCTA
ACCAGATGAAAACGGTATTCTCCCGTGTAATGACTGGACGTAATGAAACTGAAGATGGCC
GTAATATTGATCTACTATTCGGTAATACATCGGTAGAAGAACGAATGGTTCTATCTCCTA
AAATCATTGCTACAACAACTGGTATCTTGTGGGCAGCTGGAAAACATCTTGTAGCTGTAT
ATAGAGGAAACACTAATGCAAAAGCTAAGTAATAATGTAGCACATGGTCAGAACATTGCT
GTTCTTCATGCTGCTGTTAAACTAGGTACTCAAGCAATTCTACAATCGCTTGGTGGCACC
ACCAGCTCCCCATCTCTGAATGGGGAGTTTTTAACTGAGGCTGATATTGCAGCAGCTTTA
AACGCTCGCTTAATGAAGAACCTCAATATCTAAGGAGTGTTAAAATGTTCTGTAAAGAAA
CCCAAGCTGCGATTGTTGGCTCAGCACGTAGTCTAACTGAGGATAACCTTGTTCTAGTTA
TCCCTAATGAAACATATCCCCTTGCGGTCTTTGCAGATGGTTTCAGTGTAGATGGCAACT
CTAATAAAGTTGCCCTCTCTGAAGAACTGGAAGCATCTGTAGAAGAGTTCTTCGAGGAAG
CTAACCAAGAGCAACCTGTTAGTGTAGAAGAAGAGCAAGAAGCTCTGATTGCACATGTCT
GCTCATCATTACAAAAGATCCAATTCAATACGCAGAATGTTATTTGCCCTGCAATTGAAT
ATATGGTACGTGTCTTTAATGAGCGTCAAAGCATTAGTTGTCAACCAGATGTAAGCGCTA
CATTCTACCACTATAATGCAGTTCACAGTGATCCCCGCCTAACTGCACACGTTAGTACAC
GATACAATAATTTACAGCCGTTACCTGAGTATCGTACCTTTATTCTAGAATCAATCGATG
CTAATGAAATTATTCGGTTAGTATCAATGGGTAATCCGCATCTAGATCAAACTGAAGTTG
AAGAATGGCTACTATCTATTCCTAATAGCGCTGCTGTTATTGAAGGTGTATGGAATTCTA
TCTTCAATGATGGTCGTCTTCTCATTCCAGGTGAACTTGAATATGTTGTGGGTAGATCCT
TCCCATTCACTGTTGATTCACTAGCTCTGGCTTACATGATTTGCGGTCACTACATCGATA
ATCCGGTTGAAGTTATTGGCGAGAGTGATGTTGACCTAGAAACATGGTCACTAACTATCC
GTAAACTACATGAGATGTTTGGATTCTATCTCAAGCGTGCTTATGAACGTCGTCAAGCTG
ATCGCGATGCAGGTCTACTCATCCTAGCTTCAGAAGCTAATAATGTAGTTGCTAATTCTC
GTTGCAAAGTAATCATTAATGGTGATGTTGCACCTCAATTTGAATTAGCAGGTGGTGACA
TCCAAGCTATCCTTGGTGCAGCTGTAGCAGGTAGTAATAACATCTACCTAGAGCAGATTC
TTAATAACTCAGAACAGTTTATTCAACGTTGGCTATCGGTATATCCGCTGATTAAACAGT
CAGCTATGGATAAAGGTTTACGTAAACTACGCAATGACATCCATGAATCATTTATGGATG
GCGTTATTAATACTGAACTTAAAGATCGTCATGTTAATGAACTCGAATATCGTATTAAAG
AAGCAATGAGTACGATGACGACTGAGGATCTTAAGAATCCTTACATTACCTTTGGTAAAC
TTATCTGTAAAACATTCTTTACAGAAAATGTTTACTGGCAATATCTGTTAGCCATTGATG
AATTTAGTCAAATGTATCCTGAGGCCACTCTACGTGAACTCAATACACAATCACTAATTT
CGCTAATGGCAGCATGGCTATCTCAACAGATTAAAGTTGAGCGGTTCACTGGCGTTGTTG
ATCCATATGCGAAACTGCCAGTAAAAGAAGTTATTGAAACTGTAGAAGATGATGACACTG
AAACTTTAGATGATGTTGAAATCGAAGAAGAAGTTTCCAATTAAATTATCTTAATGGTTA
ATAATCATTAAAACATGCAATCGATGATAAACTCCGGTATTTGGAAGGATACCGGAGTAA
TACAGCGAATATTTAAAGGTGCATGATTTTGATTAAGTATTTTAAACTTTACATAAATGA
ACAATTAGGACATGATAATGGACGTACGTTCTCTTCGACGGGATCCAGAACGGGTCCACA
GTCATCTAACTGATTTAGAAGATAATTCAGTAGTAACTAATGCCCCATGTAAAATACAGA
TTCCAGAAAGATTTACAGGAAGACATCTAGCTGTTATTGGCAGTGAAGTATTTACTATTG
GTATATTTCCTATTATTTTTAATGATGAATATTACGCAGTGAATAATACCATTGCAATGA
TGCGGATAAATCCAGTCTCCACTGAACGCGTCCACATCAATGGTGAAGTTTATCTAGAGT
TTCATTTTGATAAAGGCAGTAAGATTTTCCAGAATACTTACTTAGTTGTTAATGATACAT
TAACTTACTATGTTTATGATGAGTTAATTAACAAAGGAAACTTCCCTTGGTATATTAACT
ATTATGACAAAGCCAAATTATTTGAGACAGCTAATTTACACGCTGGTGTTAATCTTGGTG
GATGGCCTACATTACAACTTATTATTTCAACCACTCAGCGTGCCTCGGATGATATGTCAC
AACTATATCGACATGTGCTAACTAGACACAGTGATATCGTGGATAATCCACCAGTCGACG
TACCATTCCGTTCAGTTATCTGGAATACTTCAGACACAACATCAAAACTCAATGGGGCTT
ATTTTGATCAAGCTATCACATCAGCACTTGTTAACCCATCTGAGTCAGTAGAGCTTATTG
AAGAACTCCTCAGGACCTAAGGAGTTGCGTTATGCAACCATATTCTAATTTTTCTGGTTT
GCAACAGCGTACTAATAACCAAGTAGTTATCGGCAGTGATATGTTAGCTGGTACTAATAA
AAAAGGTGTATTAGTGCCTGATGCGGATGGTTACTATCTAACTCCACTAGGAGCTTATGG
TACCCGAAATTCAGCTGGTATGTTCTATGAAATGGCATCCGGCGTATCTATGTTTAATCC
AGACTCACCTTTAATGCGTCGTGTTAAAAAGGGTGTTCTATTCATGGAATACAAACACCC
TGAACCCTATCGTAAAGATGGCACACGGATGAATGAACACGAGTACCTAATGCGCATTCG
TCAAATCGATGATGATCGTGTGTGTGCGCATATTAAGGAACTTATTCTGGTTGATTCTAC
AGATGAGAAAGGTCTTCCTATTAAACTAGTACTTGGCAAATGCAAACCATACGGTCCTTT
TGGTAAGTACTTCGAAGCATCTATTACCAATCCTTCTCAGAATACTTATTGCTCAGTAAG
GTCAATAACCCAAGATGATCCTATGCGAGGTATTAAATATACCCGTGAGATTTCAACTTG
GGATATGGTTGGGGAAGGTGGTATTTACGGTGCTAATAAATGGAATTCACCTGCATTAGA
GAACTATGAAGATCAATTAATGGTTATCACGCCTGATACTCTATATCAGGTACAGCGTGA
ACGTGAACGTCAAATTAACATGGGTTTTGAATGTTCTGACATCACCAACGTGACGGATCT
AGCAAAAAGTCTTGGCTGGGATATTTCACCAGTTAAACATAGACGCCCTGGCTTCATGCG
TTAAAAAATAAAAAAAAAGACATAATGCCCTCCCGCAATGGGAGGGCTTATGCTGTCATG
TTGGAATAGCGATCATTGGATACATATCCCTAAATAACTCTAATGCTTGCGCTGGCTTAT
AAAAAGGTGCCACAAGCATAGGCGTCCTATATGTATCATTACTTAGAAACAGTGTAGTAG
CATCATCAGAGGCTGTTTGCAGTACACCTAAATTAAATACTTTTTGAGTATTGCGCAATG
TCCAAAACCAATCTGGTTTTAGCTTGTCCATGATACCTACTAACTTACTTAAAATAGTTG
GATAGTCCTCAGGTTTTGTACCTTCAGGAAATACATAATTAGCAATTATCCTATTTGGAT
AATCTGGCACATCAGCGGTAAGGGCCTCAGCTCCAGAGTGTGTAATTATTCGATCAATTA
CATCTAAATCAATATTATTACCCAGTGAGTCAGCAGTACGTTTAGTTAATGTGAATCTAA
AATCATCATTATACTTGTAATCATATCCAACCCAGTTGCCTGACCCATCATAACGCTTTG
TAGCACCGAATGTATCGTCGATCAGGTCAGGGATACCCCATTGTGCTACTTCTTGTAATA
CCCCTCTTAAAACGGCTGCCTCGGCCCCTAAATCAAAGAACTTAATAATGGCGTTAGAAC
CAGACATGTCTTCTAAGAAACTAAGAATAGAAGACATGTTTTGATAATTACTGTTTTGTT
TATAAACATCCACTCCATTAATGATCATCTTTACTTCATTGGCCATTCGAGTGGCTTGTT
GTACATACCCTGGACCACCATTTGAACCAGTTAGTTCACCGAATATGACATTCTCTAAGC
TTTGGGCAATATTCTGTAAATCTGATCTAGAACCTTTTAAAGCACCTTGAATACGTCGTG
AAGCATCTGCTAGATTAATGCCGTCAGATGGTATTAAATCACCAATACCGCGAACAGCTT
TAAGAGTCCGATTAAGGATACCGCTAAAACCTTGTTCAAAATTCGAAATACCAGATACTA
AAGAATTTCGCACATCCGAAGATACTTCGGTATATGCGTCGATCTTAGCAACTTCATCTT
TACCTGTACCTATAAATGAAGGTAATGCAAAATCAACCATGGTAGTCTCCAACAAAAAAA
ATAATCTATAAGATTGGGGCCAGATACCCCAAAGGAATATCAAGCCCCAATATTAGAAAC
TACTATTTTTTATAAGTGATAAATTTTCCAATATGCTTGAATCCTTTACAACTAATAGTA
ACTTTAATCGGATATTTAGCCCTCATTACATAAATTAGACGGGATCCATTAAGCGGTGCA
TACAGTATAAAATCATTATCTACCATTTCTTTATGGAACGCATTAATCTTATCTTGGACT
AGTTTAATATAATCCTCATCATTAATAATGCTTTGTTTATCTGAAATAACAATCTTATCT
ACAGTATCGCCTTCGGCGCATAGTCTTTCATATACAGCAGTACGGATAAATGTATTAACT
GCGATATATGTATTTTCTGTTACTTGTGTAGCTTGCGCACTATAAATATCACTAATATAG
TAATTAGGTTTATATGGACCAAATGGATCAATAATATCTTTAATAGATACACTCTTTTTT
GGTAGAGTATGGGGTGGTTCTAAAAATGAAACACTGTCATGTGCATCGATATCTAAATAC
TTATTGACTAGTGGGGCAACGCGGTTGAATGCTTCTTCATAGATAGCATACATGTAGTCA
CGGTAATCTGGTAAAGACGCATAATAATTACGCTGAACAGCAGTCTCGGCACTACTAGCA
TAATTATAAAAAATACCCGCGTGCCTTAAAACATTTTTAAACTCATCTACACCTAGAATT
GAACTAATCCAACTAACATCATTATCGTGGATAAATATAGCTAGACGAAGGTTTTGCCGT
TTTGGGTTAGTTTCATCTTGGAATGGATATGTTGTATATAGTTCAGTGATAGCCTGTTTA
ACTACATCTGAAGATTCATTGATCAGTTCCTTTGAGAATTGATCAATTGACATAATCTGT
CTTTTCTGATATAGTACATTAATACATGCTTCTACCTGTAATAGAAAATTCATCTTTTTA
CTAATTTCGAAACTATGACTATTCTGCATTACACTATTCCTATCTAATTGATTTAATTAC
TTCGCATACCACATCAAACCATTCTTTTTCCTGAATTGTTGGATTTGGACCTTGTAGTGA
TAAAGCGCCGAGATTTTTAGCTTTAAATAGAACGAATGGTCGACTATTAAGATTTAATAA
TAGGGTATCACGAAGGATATTAGTAAGTCCATCTACATATTGGTATTTTAAACCATAATC
TAGTTTATAATGAGAATTGACTTCTGGTTGAAGAAAAGCCTTTTCTTTTTTACCAGTGAT
GAGATACATCCGAAGACCTTTGAATGAAAAGAAATTCTCATTATCAAGTGTGGTCTTCTT
CTCTTTTCCATTAAACTGAAATAGATCAGGGAGCGGTGTATTACCGCTCTGATCTACAAT
GATACTATCTACAGGATAATTATTTTGTACCCATGGGGTTTCTGGATTATTCATCTGAAT
TAGGCTCCACCCAATGAGGATCAGGTACAGGAACAATATGTACACTGTCCTCTAGTTTAT
CTCGAAGATTAGGATCATCAGTCCATTGTAGAGAAAGAGTATATCTCTCCTCTTTAGGCC
GTAGCAGTAGGAATCCACGTCTTAATGTATTCCAAGACATATTGGGTGATAGTAAACCAC
GCCTTTGTGTATTAGCCAATGAAGTAAGTTTCTTTTTATCTAATCCAACAAGGTTGACTG
GATTATTTAAAAACTCTTCCCATAGCTTATCCCACTTCTCCTGGTTAATTCCTTCTTTCC
AGAGAATATGCCTATACAGATGGGCCATTGTAGTTGTAGCTGGTTTTTGATTCTTAAAAA
TTTCACATTCTTCATAGGGGAAATCATTTGCAGAGCAAGTTACCTCATCTTCAGTTGGAT
CAATATTGATCGTATAAACTGAATCAGGTTTTGATGTACCCCAAATCAGACGTACCTCTA
ATGTTGCTTTAACAGGTGATAAGAAATCAATAGCTTTCTTAAAGCCATCCCAACTCATTG
TATCAGCTTTAAGTGCTTTATTAAGATTACCTTTATCTGGGTTATTCTTGTTATGTGGTT
TTTGAAAATATGCATTCAAACGTTTATCCCAAGCAAACTGTGTCATATTCCTCCACAGTA
ATACTTCTCGGAAAACATAACACAAAACCCCTTCAGCCTGCCTTAATCCTTTTTGAGGAT
CGCTTAATAAGTCTTTTACAGATTCCATCTTTTTTACACCTACGGTGAATTGATAATAAA
CACACTTTATATTAATTTATACTTGTATAGTCTGCACCTCACAGTAACTTAATAATTGCT
CTTGATAGTTCAATAATATCATAGTACATTCTATCCATCTGACGCTGATATGCCTGATGG
TTAATCTGTTCAAGATATTCAGTTTGTTGCCAAATTAGGTCACGCATAGTTTTAATAGAT
TTACATACATCAATAAAACTATCATTACTGTTTATAAAATATTCATCAAGGTCAATTGAT
TTCCTGTCGATAAACTCATTACGGCACATTTCACTAATCTTTAGATCATCTTCATTTGCA
ATATGATCATTTAACCTGGTTAGAGCATCTAATAAATAACCAATACTGTAATGTCTTAGA
TAGACAGGAACACCCATCATCTTCCTGATTGTCAGAGTTTTTAATTCATCCAGATTAATT
AATTGCCAGAAATTATTAATAGCTTTTAATCGTTCTTCACCTGAAATAATATCTTTATCT
ATCCGTTCTAATTCAATTAGAAGTTGTTCTTTACCAACTTCCTTTTGGAAGCCCCGGAAA
TATTGAATAATTTTCTTAAACATGATATAGGACCTATTTTTCAGATACTTAGCATATGGT
AAGTATGTTAGGTATTACTATTACTAATTAGAGGATAATAAAATGTTCCATGGTGCAGAT
TTACCTGATGAACCTTTAGAAAAGAAATCATCCGCTACAGAAGAAAAGGTATATGCTGAT
CTAGAATATGAGCAGCAGATACGGTATACACAATCAGTCAAAGAGAAAGTACTAAAAGCA
GTGTTAGAGAATACTACAGGCAAAGTACCTACTGACAAAGAATCAGTTGATATGATTCTT
AAATTAACAGATTCAATGGATAAGACAACAATTGCTAACCGGCGTATCCAAGTTGATTCT
GAAGGTGCGCGTTCTAACCGTGAACTTGCTGAAGGCTTTGCTGAATTCGTTAAGATGCAG
ATGAACCGTAATCCATTAATACGTGATCCGAATGATGGACCAGTAACACCACGTGAGATT
CCAGATATCCCTGATGAGGAATTTGAGAATTACACTATTGAAGAAGGTGAAACTGAAATC
GGTATCATCATGGAGGAGTCCAATGCATTTATGGAACGGATGGATGCTGCACGAGAAAAA
GAATTAGAAAACGACTGATATTAACGACATATAGCCCTCCCATTACGGGAGGGCGTTATG
CACTTAATACAAATCGATATCTAATACATCACCAACTGTATTTACGATTACTTCGACAGG
TGGTAGCTCAATATTACCAATTGATTTCTTAATAACTTTCATTTGGTTAATAACTTGATT
ATGATCAGATGTTACTTTGAGAGCTAATAGTCCAATAGCAGTTAAACTTACAATAGCAGT
TGAGTTATCTGAAATAGTTTCATATGCAGCTTTACCTACTTTCTTAATAGTTTTAAATAG
AGTAGGTTTATTTTGACGAATTAGTTTTCGTTCTTTTGCTTTAGCTAGCTCATCATCGAA
TTCTTTATTAAAGCTACGGCGTTTACTCCAATCATAAACACCTGGTGAAACTTCTTCAAA
ATTGGAAGAGATTGCACCAAAATCACTAGCATGACTAAACATCCATTTAAAGATAAAGCG
ATTAGAAAGAACCCACGCCTGTAGATTAGTTTCTGCATCGTTGATTAGTTTAATAGCATC
ACTTCCAGTAGCCACTTGGCCATTAGCTTTAACAAATAAAATCAGTCTTACAGTAGTACC
ATTGATTACTGTGTTATTAACATTTGCAATCCTGGTATAAATTTCTTTAATAATTCGCTT
TTGTTTATCTAAATCAGCACCATTTAAACGAATATTAAATTGACGGTCTAATACGGCCAT
TGCAGTTTCCTCTAGGGTTATTGTACCTAAAGTATACTGCATGACCGTATTATTTTAATT
TTTTACATGCTTTATAAAACCGTGTACCAACGTACAAAATAAACCCGCCAATAACAGCAA
CTGATGTGATTGTTTCCAGCGGCATGCGCTTAGCGGGTTCTTCAATTGGTACTGTAACAG
CTGGTAATTCAATTCTTTTCTCAATAACTTTTTTACCAGTTAACTTATAATAAACATCTG
GTTCTAATGTTATCGATTCAAGAATCTGATTACTACATGGATAGCGTGTGACGCCTCCAT
GTCCATCTGCAACATAGAAGCTGAATGTATCGCCTACACGATGTAAGTCAACCACACTAC
CCATATATGTCATATGAGCTGGATAGAGTTCTGCCTTGACCTTTCCATCGTTGAATAAGT
CAATGACGGAACTCTTCTTTAGCTCACCCATTAAGTTTGCTAAAGTGAATGCCGCCATTA
GCGATAACCCTATTATTTAATCGACTTGTTGAGCACTATCATCAATATCGATTTTATCTA
GCTCATCAGTTAGAATATCTACTTCACTGTCTAGTACCGTATCATCATCAGCTACGGTGT
CATCAATAACGGTTGATTTAGAAACGGCCTTATATAGATTAATAGCGGCAACAGCGAGGG
CACCAGTTGAGATAATTGCAGCTGCGATCTTTAGTACTTTCATTTTCTATTTTCCTAATC
ATTAAGTTTTAATAAGTATCCAATATAGATACATGTCTGTAATATAGATCTTAAACTTTT
TTTAGTGTAATAAAAAAATTAATGCGATATAAACCCCCTCCCGAAGGAGAGGGTAATATG
TATATCAGTATTCCATCGAAGTTAATACTCGATAATGATCTAATAGTAAATCAATAACAG
AGTAAAAAGAATCACAGTTGAATTGCGTATCTATATTTGCAGATACTCCAGTAATACGAT
TGATAATATGGAGACACTTAATATCTCCACCACGTTGTCTGATAAAAACAACTGCCCACA
CTGGCCACGTACTATCTGGTGTGGTGTCTTGAATAACTAATCTGTAATACCCATCATTTA
CTGTTTCAATGGATGCTTGGATTGGATCATCTGTATAAGCCGATTCTACTTGAACATATT
GATCATTATAATAAAACATTTCCTTAATAAGTTTAGCACAGGCATGTAATGTCTCCATTA
CCTTAGCTGAACTATTTGGATTATATTGTTGATTAAACATGTCATTAACCTCTTAAGATA
TTGTATGCATAGTAGTAATATATATCTTAATTATTTTTTAGTGTAGTTGTGAAAACTTAG
AGGAATATACGATGCCTAGTAAAAGTAAAAAACAACATAACTTCATGGCTGCTGTAGCCA
ATGATAAGAAGTTTGCTAAGAAAGTTGGTGTACCACAATCAGTTGGTAAAGACTTTGTTG
AGGCTGATAAAAATAAAAAGAGTTTTGAAAGTGTTTCACTTAATAGCACTTATAAAGATA
GTTACTAATATAATGCCCTCCCATTGCGGGAGGGCTTATGTTGCTTATAATTATTTTAAT
ACTAGATTTTTATTAAATACTTCTGGAGTACTAATATTAGCTGGATCAAGTACAATAAAT
TCAGCTTGGTTAAATGCACCGTGTGTTGATTCAAAGTGGCTGATGAAGAACACTTGATTA
ACAATGCCATTTTCAATTAACCTGTTAATGAATGGAATAGCATTGGCTCGATGTTGTTCA
TCAAAAGTCCTGCTAAATTCATCTAAGAAAAGGGGCATGTCATCTAGATGTAAAAATTTC
AAGATAGCCATTCTAAAAGCAAAATCAACAATCTCTAATTGTGAGTCAGACCCGTATTCA
ATGTCCGGTGGCTGAACGTCACCACCTGCAATATGCATTGGGAACTTATAATCCATGCCA
TCTTTGTCAACATTAGAAGGTAATACTTCTAACCTATAAGTCCAAATCTCATTGATGTAT
GCATTTACTAGTTTAACTACTCCTTGTAAGAAACCTAATAAATAGCGACCAAGAATACCA
CTTTTTGGTGATAATGCTTTTTCAAGAATAGCTAAATCAGTAGCCATTTGTTGTGCATGG
TTAACTTCTGATTCTAAAACTTTAATACGTTCTTCATGACGTTCCATTTCACTTAAACGA
CTATGCACACTAGATAGTTCTAAGTTAACAGCATCAATCTCTGCTTGGTATGCCTTATCT
ACAAAGTTATCAGCTGTATCCACTATCTTTTTAATAAACTGCTCCCATCGTTCAATAAAA
GAATGATACTTATCTTCCCATTCACGAATAGCATTACCAGCTTTATTGAAATGAGATGCT
TTTCTTTGTAAGTCAGCTGCTACATGTGTTTTATTATTAATTTCAGTTTCTAGTGCAGTT
GTACGTGCCTTAATGAACCCTAATGCATCTTGATCCATCTCAGCAATAACACGTAGTCGT
TGAGCTAATAAAGTAGCTTCCGCATGAGCTTGCTTAGAGACAATGAGTTGCTGCATTGCA
TCTCGCCAGTCTAATGCATCATTTATATATTTTGCTGGTGTGCGATACATTACGACATGT
TCAGCTACATAGTTCCATAATGTATCAAATTCGTTATACTCTTTCGTTAATTGAACGTAG
CCATGTACATAACTTACATATTCATTGAAGTCAGTCATGTACTGTTCAAGAACCTTGATC
TCATTTGAACAGCTATCCATCGCTTCATCTAACTTAGCTTTGCGCTCTTCTGCTATTGTG
ATATCTTGTTCACTAACACCAGGTTTAAATGAATGAGTGCATGATGGGCAAGTAATAGAC
GCACATTCCTTAAATGAATTTAGGCGTTGATTAATTTGTAAATACATGCCTTCCATGGTT
GAATATTTACTTTTTAATTCTTTTAGCCTTTCTGCATTTTTAATAGCAGTTTCTTTACTT
AAGTAACCATTCTCATTATTAGGGAATGTAGTGAGAAGTTTGATCCAACGATCTGAGAGA
TCAGTAAGTTTACCAGATGGATCACCGTTAACAGAAATGTCAACCAAAGGAAATCTACCG
GTATACTTTTTATATTTACTAATAGCTTCTTCAGCAATGCGTGTATGATGTTCATGTCTA
TCCTTAAGTTCTTGTATTTCTTCTGGAGACATACGTTCAGCTGTATTCGGATTAGAGCTT
TTTAATTCCTCTAATTCTTCAACTAAACGTGCAATAACTGCTTGTATAGATTTATATTCC
TCAATCTTCTTATTATTTAGTTCTAAGAATGATTCATACCCATCTACTTTAACTTGAATA
GGTAACATTGGAAATGACATTAACAATGAACGAGCTTCATCTAAGATAGAATCTAATTCT
TTACGTGCAGTAGTTTCATCTCTAAAATGAAGGACACCTTTCTTATCACGTGTTTTATAA
AGTAAATCAATACGGCTAACTAACCGTTGTTTCACTTCTTTCAACTGTGTCATTTCTGAA
TCATTCAATACCCCATGGTGTTCTGTGACCAATCGCTTATTAATTGTTTTAACAGTACCT
TGCTGGTTAGATAATTCTTTACGTACTTTGTTATAAACATTAAATGCATAATCAAGGTTA
ACTACAGACATTCTAGTTAACCATGCTTTACGTTCATTAGGTTTTAACTTTGTAAAACGC
ATCTTACCTGTCAACATTTCATGCAGTTCTCGGTTTAACCCGAAATGCGCCATTACAAGT
TCTTCTTGTTCTTTAAAGGTATGACCACTATTTAATTCTTCATCATTACATAAAAAGGAA
TGATGTCCAGTGCCACGGTTATATTTAGAAATTAATTTATAGTCTTTACCATTTGCATGA
CAATGGAAATGTTTCTCGCCACCTTGCTTGAAGTTTGAACTGCTAGATACAACAGGTGTC
AGTTCACTAATAATAGATGATTTGCCAGAACCATTACTACCAAGTATAACCATCATCTGT
TTGCTTGGAGTCCATTCAAAATAATGTGTATTATTGGCCATCAACCTAGTATAGCCAATT
AGCTTAAGTAGCTTGATCTGCATGATTAACCCTTAATAATGTCTTACTCTATAGAGTTAA
TAACCCTAGTAAATATTGGAGTTTATAATGGACGAAGCTGTGTCACTCTTATCAAATATG
CAAGATAGTGAAATACAAACATCAGAATTTAGGTTATGGAGCATTGGACGCGCTACTGAA
AATAAACCACGTAATTCATTTACACTAATGGTACTACCTATTGAGTCGGCAACTGCTACT
GATGGTGAAACAACATTCAACCCTGTTGAAGAAGTAGTTGATGGTGTAGATGCCGATGGT
AGGGCATATACTACTAAAGTTAGTGTATCGCGTGATATACCTTGTATATGGTTACCTAAT
GAAGATAACCGTGCAACGCCACCTGATGTAATGCGTGGTGAGAAAATTGCAATATACAGG
TTAGGTGATACTAGTCAATTCTATTGGCGGTCAATGGGTTTATCAAATGATTTAAGGACA
TTAGAATCTGTGGTGTACACTTTTAATGCGTCTTTATCTCCTGGTGGTGCCGGTAAGAAT
TTTGATACCTGTTATTTCATGCAGTTCTCAGCTCATGATAAACACGTAACTATTGGGACA
TCTAAAGCCAATGGCGAGCCTTATCGTTACTCGGTACAAATCAATACAGGTACAGGTGCA
GTCTATATACTTGATGATATTGGTAATAGGTTTGAATTGGTTTCTAAAGATAAACGACTG
ATGTTAATGAATGCTGATAATTCTTTTGTGAAAGTAGAGAAGAAAGCTATTGATTTGAAT
GCGGATCAATATATCAAATTGACATCAGGTGGTTCTACATTAGAATTAAATCCGACTGAA
TTTAAAGTTAACACAACCAATACTTCTATCAAATCAAGTGGTACACATGTTCAAGAAGCT
GGTGGAACTATGACACACAAAGCAAATGGCAACATGTTGTTTACTGCTCCACGCTATGAC
TTTACTTAAGAGGTTAATATGCCGGGAATAGCTGTTTGTAATATGGACAGTGCAGGTGGT
ATTATATTACCTGGTCCTAATGTTAAGTGTTTTTATAAAGGTCAGCCATTTGCCGTTATT
GGTTGTGCAGTAGCAGGTCATGGTAGGACTCCGCATGATTCAGCTAGGATGATTCAAGGT
TCAGTAAAGATGGCTATTACCGGTATACCTGTGTGTTTGCAAGGTAGTATGGCTAGTTGT
GGACACACAGCTACTGGTCGACCTGATCTTACTTGTGGTAGTTAAAGAACATAATGCCCT
CCCATTGCGGGAGGGATTATGCTGTTAAATACCTTCATTCTCATAGTGAGTAATGCAGTG
ACTAACAAATGTTAACCAGACTTTAAGAGTTACTGCTAACCAATGGGTGTAGTTACCAAC
AGGTTCTGCTAATTTTCGGGATATCTCAGCGAGCATATTAATTGCCTCGCCATTTGCCCT
TCCTAACAACTCACTACGTTCAGCTAGCTGTCGAATTGAATCATCTACAACTACCTTAAC
TGTTTTATATTCTACTTCGCCTTGCTTAGCACGTTCAGCGATCGTTAAAATATTAGCAAT
ACCAGTTACAATATCACTTAGTGTAGTTACAGAAGGTACTTCTATATCTCGTTCAGTGTT
ATCAATTGCATTGATTTGTAACTCTGTAAACTCCATCGTAAATGTATTGCGAACCATTTC
TAGTGCAGATTGTTTATTAGTAACTGATGTGGCTATATGGTCAGCATCAGTATAAACAAA
TGCACGATTAGCTGGTAAAATATAACTTCTATGGGTACCAGTAGAAGTAGTCTCACCTTT
CTTTAGTGGTTCATCCCCTGGCGGTGCTTTAAAATCACGACGCACGATATCGGCAAAAGA
TTGTAATAACTCATCAATGATTTCTGTAGTAGTATCTTCGCCTTGATTATCTAAGATATT
CAATGCCTGTCTACTAATATTTCGAGCAATATTAGAAGTCATTTTAGGGTAAGTCATTAA
AATATAATTAGTGACTTCTTTGATATCTAAAAGATTATCTGGTCGATCGCCAATGAATTC
ACCGTTAATAGATAGCCGCTGTAAACCAGAAACAGTCAGTCGAGTATTAGTCGTACGATT
ACCTAGTTTTGCAAGTCGCCTAGATAACTTTTGCTGTGTTTTACGCACATGTGAAATACT
GCCAGTAAACTTGACGTAGAATGCCGTAAGTGTTTCGATTACCATCCGCACAATATCACG
TGCAGCCTTAGCAGCTCGAACTAATGCATCTTTTAAAGCACGTAGTGTAATTATACCGCC
TTCTTTTATGGAGAAGGCGGGATCGATACTTTCTAAAGCAAATGTCATTTGCTTTAGACA
GGTCTTCAGATGCTACGTCACGTCCATCTAGCAGATCTTGATAACGAGCTAATCGATGGC
GATATGCTTCAACAGAAACAATAGCCTCTTTGAGTTCATTATTGGTAATTAATAGTTCTG
CATATGTGGACATTATGTTTACCTATTCAGTTTCCTTGATTTCACTATCAACTAGTTTCT
CAAGTACGGTGATCCAAATCATCACACATTTAAACAGATCATTACTAGCTTCAGTGGTGC
CAGTTAGATAAGCCTTACCGACATTAGTTGCTTCTTTATCAGTCATCTGTGCAATGATTT
CAGCAGTTGACTCTTTAGAGATACCGCGAATATTATTTAGATGATTTAGAAGGTTATGAA
CTGCTTTTAGCTGAGCCATAACAACATTCTTATCAACTAATAGTTCAACAGTAATTCCAT
AGTTGGGTTTACCCGGTGCATCAAGAATGGAGAAGTCTACCGCAGCTTTGTCTTTACCAA
AGGTATAAACACGATTACCGGGTACAACCGGACTAGCATAGCGAACACCATTAGCATTAA
TTTTGGTAGCTGTTCCAAACATACCTTTAGCTAAAGCCAGTGCTTTATCATCAGTTGTTT
TTCCAGCTAAGGCTGCTTTAAGAGCACGCAGTCCATTCTTTTCAATACTGTCAAATAGAT
TACGGATTACAGTGACCCCCTGAACAGGGTCTACACCCACCCAACTCTCACCAGTGTCAC
CTAGAATCAGATCGCGTGGAATTGCGATAGTACGAACTTCATCGGGTAGTTCATTCTCTT
TAACGCGTTTCATGATCTTATTAACGCGTTCTATTTGATTACTGACATTCTGGCGAATAC
GTTGATAAGTTTCTACAACAATGCGAATTAGCTTCTTAATGAAATCTAATGCAACAGTAG
ATACTTCTTTTGCTTTAGCTTGTAGATTATCTAATACTTCAATAGAAGCTTTAGGCCGAG
TTAGCGGTGATATAAAGGATTCCATTGATGGCACAGTGTTATTAAAATATTTATAGTCAA
ATGACTCTAGCGCGATGCTAATAGCTTTAGCTAGATCATGATCGACATCATGCCCGCGAT
GATGGCGGTTACTAACTAAACCAATATAGTTTTCTAATGCAGTTTGAACAGATTCTACTT
TAGTCAGTTCATTTACAATTGTAAACATTTCAACTGATAATGATTCTAGATGATCATCTA
GTTCACCAGTGATGATATCTTCAGTAGTTTCCAGTAGTGCGGTGGCATCATAGCCATGGT
CTAGCTGCTCAGTCTCATTGGCTTCCATAATAGAAATGTCTTCATTCGATGGGACATCTG
CAATTTGGTGTGGTTTTTTGGTACTAACATTAAGATCTTTAGTATCACTTACTTTAGTGA
TCATTTCATCAGCTGTTTCAGTGATTTCGTTTTCAACACGATCACCTTTAGCTACTTGGA
ATTCTTTTAGCCCTTGACTAACATGAATCTTCTTATCATTGATCTTATCAGCAGTTCCAT
CAACATCTGGACCTTTGCATACGTCAATTGGGGTATTGGTAACGACATTGGTAACTTCAT
CACATGGATTTACTTTACCATCTTCATCATCTTTTAATTGTTCTGGTAATTCTTTACCAA
CATCAAGATTGACATTCTTATCTTCAGCATCAGAAACGCGCTCAAGACTTGCAAAGAAAT
CCATTGCGGGAACTGTTTTTTTCTTAGCCATTTTAAAACTCCGGAGTATTAAAGTTCATA
AGGTTGTTAGGAGATAATACCATTCTCAATGAGAAGATTCTCTGCAATTGCTAACCTTTG
TGATAGACTTGTGTTATCTGCTTCTAACTTAACAATTCTAGCGTAATCAGTTTCACGATC
TTTGATTTTCGCTTCACGTGCATTTTCTGCTGCTTCGTGTTGTTGTGGTGTCACGTCTGA
CATAAGAGGCATTGACCCAAGATTTATAACAGGTTCTACTCCAATCACATCAGAAATAAC
ACCGGCTACTTTAGCTTGTAGATTAGTTAAGTCTAATGTTACTGGAAGAGCCCCTAGTGA
TGCGGATAGAACAGTTTGCTGGTATGGCACACCGTTCAAATCAGGATAACGTGCGATATA
ACTGCTTGGTACATATACCGGGTTATATTGTTCAGTCGTGAGAGTAACAATATATTCGCC
ATTCTCACGATCTTTATTGTACTCCGATATATCAATATTTAATGGTTTATAGTAAGTGTC
ATAAACATTTCTACCATTATTTTCAATATCGATAAATGCTCGTACAGCACTATTGACATA
AAGTGTATTGGGTTTTATGAGTTTGTCAAAAGGTGAAATTAACTCATAACGCCCTTTAGT
CCCAATTGGTGGGATCATTAGAGATTTTTTCTCAGTAGCCATTATAGACCCCAATAGGTT
AATTATTCATTCAATTGAGCACTATAGTTACTTTAAGACGAAATAATAGCGGATCTGCTT
TAAAGCAGATCCGCTTTAAATTAAAATGCTTGACCAGGACAAACCCAAATATAATCACCA
GTTGTTTGGCCATTAGGTCTGACTAATATAAATCTTTGTCCAACCGTACCGCGCACAGAA
GCATCTTCAAATGGAAAACCTCTAGGTATAACTAATTTACCATCTTTGTCTTGTGCCCAA
AAACTTAATGAGTTCCTAGTCTCTGCGCCATTAATAGGATCTTTTTCATAGAAACTTAAT
ACGAAAGTAAATCCATTCGGCAATGACGCATTGTATTTAATAACAGTGCCAATGAATGTA
GGCGTTGTCGGCGATGATACATATCCATCTTCTTCTTTAAAGATAATGTTAGTATCAAAA
TAATCTTTAACATCTAGTAGATTATAAATAACATCATCTCCATAAAGAGGGTGTTTCATT
TTTAATGGATATTTATTTATTGTTGTGTAAAATGCACCAGGAATGAAATTATCACATTTA
GATGTATCTGCCATGATTTACTCCTGTACATCCATCATTACACGTCCTTCAATAGACCAG
TATAATTCATTTTCTAATGAATTATAAACCGTTCTATATTCACATGCCTCACCTTTGTTT
AATTGACAGTCAACTTTGAGTACTCTGTTACCAACTAAACGATTACCATAAGCACGAATC
ACCAGTAATCCATTTGTCTTATTAGCAATAGTAAATACATATTTACCTTTTTTGGTATCA
TAAGCAGTAGCTGGTAAATTTACTATAAATGGACTATCTGAATTATCATTATCAGTATAA
TTAGGATCTTCAACGATAATGTGTGCATTAAGATAAGTTGAGTCAATATCAAACTTACGT
CCAGTTGCTTTAGACGTATCAATTACTTTCATAGGTGGGTAGATATAACAGAATTCACTC
ATGATTGCATCCACCATGATAGACCAAGTGTATACATCGCTTTAGTTGTTTCATTATAAC
AGATAAATCGAGTATAGCCATTATTAGGTAAGAAACCAGTACTTGTATACATTGGCCTAA
TGATACTCTGTTGATAAGCAGTGGCTATATCAACATCTTCATTAGTTAAATTCAGTATTC
GATATTCAGCACCAGCTTTAAGTTTCTCGCTATTACCAGAGGGCAGTATAACACCTTTAG
GTGACTCTACTATTTGAGCATCATCTGGTTTAGGCATTAGTACTAATAGTCTACCTATAG
CTTGGTCATAAGGTGGACAAATCCAATTACCAAATTTACCTGTTTCATTATCAACTGTAA
TTGTAAATGGTACAAGCAATTGCGGGAATATCTGATATCCATCTGTTGGTTCTGGATCAG
GATCTGGATCAGGGTCCGGCGGATCAGGGTCAACATCACCACCTGAATGTAACCATAGAT
TTAACCATCCTCTCCACCATAGTGACGAATCAATAGCTTTAAATAAAAAGTTTGGTTGTT
TCTTACCACCGGTATACGTTATATTAGATATTTCTAACCATATGTCACTAGCAGTAAAAT
TTAATTTAGCTTTATTAGCAACATACGCAACTAAAGCTTCGCTATTTTTTAACCCTTGAA
CAACCGTTACTTCTGATGCTGTCCAATATGGATTTTTAATATAATCATTAACATTAAAAC
GATTATAAAGAAAGTCGTAATAAACAGCACTATTAGTTGCATCTTTCTGAATGCGAACAA
ATGCCCTATTAGGCTCATCGATAGTTACACTATCCATTACCATTACAATAATGGACTTAT
CCTCAGATAAACCTAAATCCGGATATGCTGTTGTAACTGCTTTGAAGAAACCTATTCGTG
GATCTTGTCCACGCGGGTTATCCGCAAGATTAAGAGTGAATCTACGCATTGCACCACCTT
TAGATTAATATAAGAGAGGGCCTTTGCCCTCTCTCATTTATGCATTAACCAAATTGTTCT
ACACTCTTACGAGTTACAACCATGTAGTTAATATTCTTATAGACTTTAGCAGCATAGATA
ACATCATCACGGATCATTCGTGACATACCCATTGGTACTGTAGTGTAACGATCCATATCT
TCAGCAGCTTTCAATAGATCAGCAAAGACTAATAAGAATTCGCGTGTTTCAACACTAATC
CGGTTAATGTCTTCAGATGTGTTAGTAGCAATTATGTAATCTGGGAATACTTGAGAGAAC
TGGGTTAAATTATTACGGTTCTTTGCATTACCAATTACACTAAAACCAACAGAGCGATAA
ATATTGCAACTTAACTCATAGTGACTATCAAAGTAAGTTGTAGTATAACCACTACCGCGT
GCAGTACGACGCATTAAAGCGATCCTTTTACGTGGATCGATAATGGGACTATAGATACCG
CCTCGGAAATCTGAGTTCTCTAAAGCTAATTGATTCCAAAAAGGACAGATGATGAATTCA
GTTGTCCGAAAAAGTTCAGGTAGAATCTTCTCCCACTCCTCACGGGTATGGGCAGATCCA
TCTAATAACTCATCACAAATAGCATCTTTAATGATATCTAAGTTGTTACCAGCAACGCCG
TAAATGATCACAATCCAATAAGCTGGGAATTGCATCGTATCATCACGTGGATTATGATAA
TCAAATCGATAAGCGTTCTGGTACGTATATGGATACTGTGCACGTTTTGCTTGAACTTCT
TCAAGTTTGGTAGGTAGATCAAATTTATCTAGAAGTTTCTTAACTTCTAAAGGATCCTTA
AACCAATCGTCGTAGTTACCTTGTGCATTCTTACCAATAGGGTGAACCACCTCAATTTTA
AATTCATCATACTGACCAGCAAATGCATCATCCGCTAACCAAACACGAATTAAGTTAGGT
TCATTACCTGTAACGATAAGTGAAAATTCAATATATTCTGGTAAATATAAACGTGTATTC
ATATTACCAATAGGACCAGAACGCACCGTACTTAGCTTACCATTAAACTCAGTCATGAGC
ATTTGGTTAACTTGAGTTGGGGTGGTGTTCTTTCCAATAGTGCCTGAATTTCCTTGCTTC
AGTAGGAACTCACCAATCTTTAAAACCTGTAACTGATACTCAGCTGTAATGTCAACAGGA
ATTTCGTCCCTTACGCTATGAAAAGCAATAAAAGCCGTTGATGGTGTAGACTCACTGGTC
AGGATTTTCTTGTCTTTTGCATAGGTTGCACTGTTATCCGAGATCTCACCAAATTTTGCT
ACTTTATCGGTAGCATTATCAATGAGACTAGGATACTCGTAAAAACCTTTCAAGTTGTAC
ATGAATTAATCCTCATAATTAGAGCGCGATGGGTTTCCATCTATAAAATCAGTCTGTTCT
AGTTGTGTTAAAATAAAAAGTCTGGTATTATGAGCTTTACGTAATACCCTTGTAACAGTC
TATTGATAGAAATGCCAATACTGGCTTTTATGGGTAGGCATAGTACTAACAGAGGTCATC
ATGCTCGGTTTGACTATTAAAGCCTTGCTCTTCGTTTGGCCATTCCTTAAGAGGGCTATC
TTTGGTGATAGAACAATTAAGGAAGTGTTGCTGGAAAACAAACATGTCACGGCAATGTTA
ATCCTAGTTGTAATATTACTAGGTACCTTATTCATAGTGACTAGTGAGTTGAAGTCAGTT
AAAGCAAGCAACTGGGCCGTTAAAGCCGAAATGGATCGACTCAAGAAAATGACGCCTGAG
AAGGCTGAAATGCTTGAGCGCCGCCGTCGTTTAAATGACTTGCTAAAGTAAGTACCAAAT
TGGTGGATCACATGTCAAGAAAGTTCTTTCTAATACTCCTACCACTTATCAGTGGCTGTG
TTTATACGCCACATGAGTACCACACGCACAACACGACTGTGGTCCAATATGAGCACTCCT
CTTCTACAGATGATCATACATCTGTTGTTACAACTAAAGAAGAGGTAATACAAAAAGTTC
TGCCTACACCTAAAAAATATAGTAAGGATACTAGTAAGAAGTGTATGGTTTTCGAACTAC
CAAGAAAGAAACCGGTACCGCCAACACCGATTATTACAGAGTTTATAGAAACTCCGGAAC
AAATCGATATCTTGTTGACTGACTATATAAAAGAATTAAAGTCACATATTGTATCTGAGC
GCGCTAAAATTGAGCAAGCATATGAAGAATGGCGTGCAAAGTGTGATGAGTAATTTACTT
GATAGAATTATTATCTAGTTTAGTAATCGATCACATGAGGAAACAATAATGTCAGACGGT
ATGGTTTTATACACTGACGGATCATTCCGACAGGGTAAAGCTGGTTGGGGTATTCATGGT
TATACCTATGAAAATGTTGCAATGAAATCTAAAGCAGCCACCAAACAGCAACCAACAGCT
AAGGGATACTTAGATGTAGGTGCAGATGAGACATGCACTGTTTTAAATTATATTGATGCA
TTTGGTAGTGTAACTAATAACCCTACTAATAATACAGGTGAGCTAACCGCTGCGATTGAG
GCTTTCAAAATAGCCCATGAAGCAGATGCTCCTAGACTAACGATGCTCATGGATAGTGAA
TACGTTAGAAAAGGTATGACCGCATATTTACCCAAATGGATTAAGTCTAATTGGATTAAA
TCAGACGGCACGCCGGTAGCAAACCAGGACCTATGGAAACAGATGGTCGAACTAAAAGCT
AAATGGGAAGAAACTAATAAACCATTAAGCATCCAATGGGTCCGTGGACATAACGGAGAC
ATTGGTAATGAAAAAGCTGATGCTAACGCTAAGCTTGGTTCGGGACAGAATAATGATAAG
CCAGTGATGGTTTCGGTTAAAGGCGAGGAAATTAACAAACTTAAAAAACAAGTTGTTAAT
CCACTTATCCTCGAAAGTAGGTTACTCTTCGCCATTAACTCTGGAGAAGAACCAGATGGG
TTCTATTACACCTATAACTTAGGTCGTATGCATAACTATGGTCATAAACCAAAAGACACT
GCTAAAGATAAATTAGCTAAAGCTGATTTAATTTTAGGGCGTCGTATCTCTGAAGCAACC
TTCAGTGTATATAAGGCAAATGAACCAGATGAGTATCTCCAGTTACTAATTAATATGCAT
ACAGAAGCACTGGGTACTGAAAATCCAGAACTGGGTATCATCAATTTGGCAAACGCATAT
CGTGCAACCATTCGACAGAAAATTGAATCCATGGGGCTACCTGCTCTAGTCGTATGGAAA
GATATCAATGTTTTGTCGACGCCAACAGATGAACTCATTAGTCGGACTCTTAATCCACCA
CGTATGGCAAATGATGCTGTAGCTACATTTAATGTTATGCGCAGTCGTCTTGAAGATTAC
TTGGCTGGTAAACTTGGAGAGTCGGTTGGTATTATTGATATTACCGAGCACTTCTTCGAG
GAAGTCGCAAAGGGTAAGAAAGTTGGCTATCAACTACTTAAGACGATTACATCTAATACA
GGCTTTGTAGAAGTTCCGATCACATTTAAAGATCGTAAGATAAAATTAAAGCTTGTATTA
AATATCGATATTCCAAGTAGAAACCAATTAGCTCGTATTGGAGCTAATAAAGTTAAAATG
GAAATTCTTGTAGTAGCTGAAGGCCCATATTCTTATTCATATTCAATTGTGTTCGTGACG
GATGATGGTTCAGCTATCTATCAATCACCCTATACACAATTTATTCTCCCAAAATAGTGG
GTATTCCATGAACCTTATTAGCAGATTCAAAAAGTCGGTTAAGAATGGTTTCAGGGCAAT
TTGGATTAAGCTCTGGGACTATGCTGATCCCGAACTAAAGCGGATGTGCGTCATGGCGTC
ACTGTACTCCGTTATAGTACAGTGTAAGACAAATGAGTGTGAAAAGATCTCTCATTTGAA
TTCTAACATGAAACTAGTACGCGATGAAGCTGCCTTACGACTCCCAATTCATTATGGGCC
GTACGTATGGAAAGGCGTGATAGCAATCGATAAGATTGATAAAGCAGATCTAACCACTGA
TCAATTTTTCATGAGGCTTATTCGTAAGACTCCGTGTTGGATGAAATATGCTGATGATGC
AACTTTTTTAAAGGATTTGAAAACTCTCTTTAAATTAAGTTTACATGAGAAGTGTATTCC
AAATTGATTGGTACGACATAATGCCCTCTCCTTTTGGGAGAGGGCTTATGCTGTTATCCA
ATGATTTCACGAAGAATATCTTCAGTGGCTTTCATTGTGTTCGCAGTCTCAACAATCTTA
GTAAAGAGTGTTGAATAACGTTCAACATGGTCTGCTGCCCATGTAACCCCTTCAATTAAT
GTCTGTAATAGCTCTGGAGAGGTGTCAGAGTTACTATCCATAACATCCTCCATAAAGCTA
TCAATTAATTGACTAAGCGCTTCTGAAGCACGTTTTACATCATTTGGACGAACCCGTGAT
AAAATGCTACGAATATCGTTCATTTTCTTTGTTGCGTCAACAAATGAATTTAATGATGGG
TAAAGCTTACCAAACTCAGCTGTACTTGACCGGTTGTCACTGTCATAGAAAGAAGCATCT
TTAGCAAGATCATTGATCGATGCCATTGCAAATGAATTAGCAAAGCCACGCTTATCCATT
CGATCAGCACGCTGTTGTAGATAATATCCAAGTTGTGCTTTAACCGGGTCTAATACCGCT
GGAATAATAGTGGCCATTTCCTTAGCTTCTGTTAATAGATAATCAACATAGTCTGGAATC
TCACCATTAAAACCAACTGGTTGTGGCACGATTGTATTTGCTACTTCTAAATAATCAATG
TGTGAAACTAAATGGCGCATCTGCGCTGGATGGAACTCAGGAATAATTTGATTACTCCAG
TTAGTCATCGACGTAAATGCATTACGAGCAGCGATAGCAAAATTAGCACCGCGTTTATTG
ATAATGTTAATAGCGTTTGTTGCGAAATTAGCTTCTTGAGAAATAGTCTCAGTAATATCT
ACAAGCTGTTGTTTTGGATCAACTTGTACTAAACTAATATCATCATTAACAATATTGTCA
GATTCAATTAGATCCTGACAATCATTTTCTAGTGAAATACTTTTAGCGTATTTATGGATG
TTAAACATGTCTTAATTTCCTTGCGCAAGGATTGTTGAATATGTAGACATAAAATTACTC
TCCAAATCAATTAATTGACTGTTTAACTTAAAAAATGGAGTCAACATGTTTGGTAAACAT
TTCGAACGTCCAGCTTTCCGACCGGCTCTTAACATCGGATGCCTTATGGACATTTCAACT
GGTAAGTATGAACAAGGTAAGCATGGTGAAATGATTCTAAATGGTGGTATGGGATCACTA
ACTGGTATTGCTTCACGACCTAACAATTTTAAAACTGCATTAGGTATCTACCTCATGGCA
ATGATGCGCCGTGCTTTCCCCGGTGCACATGCAATGACTTATGATACAGAAGGTACATTA
AACCCAGGTGCTCGATTTACATCTTTAGCACAAGCTTATCCTGAGCTTGCTGCCATCGAT
TGGGAAAATGATGAGCAATACATGTTTACTGACTTATCTCGTTATACTGGGGATGAGTGG
TTCAAAATCTTCCGAGATGCATTATCTGAAAAAGAGAAAGCTGAAAAAGATTATCTACGC
ACTACCCCATTTCTTGATGTGAATGGTAATAATAAGAAAGCACTGTACCCAACTGGTGGG
TTCATTGATAGTTTCTCAAAGTTCATTGTCTCAGCTGTGTCTGAAATGTATGCTAAAAAT
GCTATCGGTGATTCTAAAGTAAACACTGATGCAATGACTAATGGTAAAGCTAAGAATCAG
TTATTTAACCAACTGCCACAGATCTGTGCTAAAACCAGTACATACATGATTCTCACTGCA
CACGTTGGAGATATCATTCAAATTGAGATGTACCCTACTGATAAACGTAATCTATCGCAT
ATGAAGAAGGATACGGTTCTTAAAGGGGTATCAGGTGGTTTCTACTCTTTACCGAATAAT
GTGTTTTCTATTGAATCTAATAAACCGTTAGTCAATAAAGAAAAAATGCCTTTATATCCA
ATTGACAATTCAACTGCTATTGAAGGCGATACAGATCTACGCATTCTAGAAGTTATTAAC
CTACGTGGTAAAGGTGGAATTACTGGTCTACCATTCCATCTCATTGTTTCACAGACTGAA
GGTATTCTTCCATCACTATCGGAATTCCATTATTGCCGTGAAAATGATTGGGGGATTGGT
GGTAATAACATTAACTACTTCGTTGAGCTATGTCCTGATATCAAACTAAGTCGAACCACT
GTTCGTAAGAAATTGATGGATAACCCTGCTCTACAACGAGCTGTTGAAATCCAATCTGAA
ATGCTACAATTAATTCAATTCCAACGTTGGAATCTTGGAGATTATGTAGTAACTCCAGCT
GAACTATATGCTGATCTTAAAGTAATGGGTTATGATTGGGATATTATTCTTAATAATACT
CGTGGCTATTGGATGTTCGAAGAAGACGAACAATTTAGTGATAAAAAATTCCTATCCACG
TATGACCTACTACGCATGCGTAAAAGTGAATATAAACCATACTGGATGACTGACGAAGAA
AAAGCTAAAATTGTACCTCTTGAACTAGCTAAAGCAAAAGCTTAATGTTAGTATAGATGT
TATCGCCAGTTTTAAAATAACTAGGGGTCAATAAGATCCCTAGTTATGTCAGGTTTGGTA
TATGAATGTTTACACTTATCAGATAGCTAAATATAAGTTAGTTATAGAGAAAGGAATAAA
ACCGCTAGACATAACTGTTAAATCAGGTGCATATCAACTCGCACCTACGTGGTTAATGGT
TATGGGACATAAGAAAGGAATTATATCAAGTGATGAATACACTGAAGCATATCTCAGTAT
GTTAGAGCAATCATTGATTGATAATCCAGAATTCTTTAAATGGTTCTTCCAATTAGAGGA
AGTAGCATTAGGTTGTTATTGTCGCGCTGGTAATTTCTGTCACCGGCATTTATTAGTAGA
ATGGCTTGAAACAAAAACAGAGGTCATTTACTGTGGTGAGATATCAAAGTAAAATAAAGA
CCCTAAGGAACTATAATGAAAACTACAGAAGATTATCGTTTAACACTTTGTCAACGTCTA
CGAATGTTTGATGTTGAACTATCAGAAGATCTATTACGTCGATTACCAGTGTGTACAGAC
GTACCCACTTTATCAGCTTGCTTTCAGCAACTATTAGCTCAATATCCTAGTCAGACATTG
ACAGCTAGAACTACCATTGATGATTCAGTCACCTTTGAAGTATGGTATGCTAATTTTCTA
GCCAACATATTACCAATTCTTAAGAGACATAGGTTACCTCCATGTACTGACCAGAATTCA
ATACCTGTTTATCAAGGGGTATCTCGCTCAAGCTAATGGTATGTTAAACAACCTTAAAGG
AAATTACCATGACTAAACGAGAATTGGTAGAAAAAGAATGCCTATGGATTATTGACATGT
TTCTACCTGGTAGTAGTAATGTTGATATCTATAAAGAAATGTTTGCGCGAATGAATGATG
AGGAATTTGAGGAGTGGATTAATAAGTTAGATTCTGGTGAAGAGATGTTGGCGTTATATG
CTCCTAACTTATGCACTAAGCCTAAATTAACCATTAAGCGAAATTATAAAGTTGCTAAAG
CGATTGGTCATAATTTATTCCAACACATCATTTTAACTGATCCAGGTACTGGCCAGGTAT
ATCGAACTGCTAATAAACATTTAGTTGGTTTAATACCAATCCGTCGTCAAGTACAGATGC
TTGAGAAAAAGAAAAGTATTCCGGGTTCTAGTCATGTGATTGACCAGAGGTCAGGGCAAG
TAACTGGTGATTCTAAAGGATCACGTATGTCTGCACCAGAGATTCAAGTTAACGCTTCAA
AAGGTCTCCGTTATTCCATGATCGAATTCATGAAATTACGTGGTGGTGACCAGAAGGCAT
ATAATGCAATGAACCGCTCTATTATCGAAACTGGTAGTGCTTCTGTTGATTCCATCATGT
CCACTTATGATGCTACTGTGCAATCTAATAAAACATTTGCATGTTATCTAAAAGGCATGA
TGCTACAAAATAACTTGGTGTAACTATGAATAATCGTCAGATTTATGAAGATATGTGCGA
AGCTTTAATTGTATCTAATACTGAGTTAGTAAATGGATACAGTGCTGAAATCATTGCGGA
TATTTTGGATTTACTAAATAACTGGAGTACCAATACTCATTGGTATCACATTGCTGATAA
AACAGTTATTAGGGATATCTGGAATTTATTCCGTAATAAAGGTAATCGTCTTCAACCTAT
CTTAAGTTTAGTTAGCCGATTTAAGATGGCTGTTAATGATTGGGAAGATGTGGTGGAATT
ACTAGCTAATTCTATTTATCCTTATCAGCCTAATTCAACTATTGTTGACCAAGAAATGTT
AGCGCGATTAATTCCACGAAAAGAATTAACTGACATTTTATACCAAAATGATTGGTTAGT
TTGGATTATTTGCCAATGTTTAAATATCCGTTATATTACTAATATGGTTGAATCTATCCC
AGTTCAAGCTAAGCCATCTAAAAAGAAGGCGGTAAATAATAATGTTTCGTAAAATCTTAA
TTGATCTTGACTCACTATTAGATACACGGTTAGGAACTATTAGGTTACTAAATAAAGATG
CAGCTAATAAGCTTGTTACTAATGGTCAAATTTATTGGCATAGGGAAATGGATGATTGGA
CTAAACTAACAGATGGTTTAGTAACTAATGAACAATTTGTCGAAGCTTATTCTAAACGTG
GTGGTGAAAATACTGCTGATACTATCAATAGTTCTATATTCACTGGTATCATTCCATTTA
TTATTCAATTAATAGCCCAATCAGATTTGAATAGATTAGATGGTATAGTTAATGCTAATT
TTGAAACATGTCTTGAAGTTAATATCTGGCCATATGAATTAACACCAGATGCTCAAGATG
AATTACTATACTGCTTACATAAGTACATAGGTGATCATATTCAGATCACCCTGATTAATA
CTCCATTAACAGATCTTACTTGTTCTAGATTAAACTCTATCTATTCAATGGCGATTATGT
ATAATTTTGGAGAATGGTGTAAAACCCATGTTCTTGAATTAGGACAAGTGTTGATGCCTG
ACTTTACGATTATTGGTCCAAAGCTATTTGAGAAAGATGTAAGTGAATTAGCTGTTGAAG
ATAAACAGTTTATTATCATGGATTTCCAGTTAAAGCATAAACTTAATATCGACTTTGATT
TCATCGATGCTGAATACTTTTCCATGTTTAAGCCATGATACCCAATAGAAAGCTCTAATA
GGGCTTTCTATGCTTTTTTTAGATATAACTGGCATGATGGTATAGTTAATAATAAATCAA
CCTTAAAAGACAAAATAAAGCCCTCCCCGAAGGGAGGGCAATATGTCGTTTTAAATAACA
GCACCTTTGGAAACTACCCAATTAGTACCGTCCCATAGGAGAGTAAGAATTGTTTTAGTA
TCGCCTAGGTCATCTACTATTAATGGAGTACCATTGTTCCACTGTAGCACTGCACTAGAT
AGTGTTGGCATAGCTACCTTACCAAGTAGTACAAGAACAGCCGTTACCATACGATCAACA
GGTCCATTCTCTAGTGTAATGGTTTGGTTAGCAGCAGTTTGAACCCTATAGCACTGTTGG
TTAGCTGGTGTTAGAGTTAAAGCTGTACCAGTGAATGTACTTGATTTTAGACTATATGCA
TCGAATGATTTATCTAGCTTCTTCCAAGTAATATTAGTACCAGTACGGCTACGTACCCAA
TCACCATCAGCGGTTACATCATTATTCGGACCTTGGAATAGAGTCCATGATTTAGCCGCA
GCATTACGTAGGAATAGTTGTCCACTAGTAGCATCAGCTTGATCAACTATAATAGTTACA
GGTGTCCATTTAGAACCACTAGCTGTTTTCTCACGTACATAGTTAGTGCCAGTAGCACCT
TCACTAGGTACTTCACCAATAACTGCGGCTGGTACAGCTACCCATTCACCATTTTGCATA
CCCATCAGAGTATCATCGGTTTGCAGAGGTGCTACATCAACCCAGTTACCAGCTACACGA
CCTTGAGGCTTAGTAGCTTGTGCAGTATCGCGTACTACATCTGAAGGAATTTCTGGGAAT
GTAATGGTAGCCCATTCAGTTTCACCACCTGCAATACCTTTACGAACGTATTGTGCATTG
ACAGTTGGATTAGGTACAGCATCAATGGCAGGTGTTGGTAATTCAATAGCAGTCCATTCA
ACAGCCCCATCAGCGGTTGCTTTACGGCGACCATAGAAAACACCAACCTCGGCATTGGGT
ACTTCATCAACAGGTAGTTCAGTCCAGCCGAGTTTGTACCGAACCATTTTCTTATCGACA
TCTTCTGGATCGACTTCACTAACTGGAACGGACATTAGTTTGTAGAATACAACCCAGTTA
GCATCTTTATACCAGACAGTGCCAGTTTTGTCGATACACCAGTCACCACGGTTACCATCG
ATAGATGTAGGCGCAGTTTGACCTTCAGGTAGAATTACCCAAGAGGATCCACGTTCACCT
TTCTGACCGGTTTTACCAGGCATACCTGGAACACCTTGCTTGCCCTCAGGACCTTCAGGA
CCCTGCGGACCCATAGCTAGGACACCTAGGCTTTCCCAAGTAAATGTACCTTCAGTATCG
ATACTTACAAATACTTCTACTTGTCCTTCAGTATTGGTGTGAATGGCAATACCTTTACCT
ACGTTATCAGCAGATGCTACTGGTTTACCAGCATCATCACCAACGTAGATAATTAATAGA
CCACCACCTTGTTTACCTTCAGGACCTTGTGGACCTTCGACACCCTGGATACCCTGCTCA
CCATTTAGATTAATTGGGCCGATATAGCCATCAATTTCCTGACCTTGGTAAGTATAGAGC
ATTGTACCTTCATTAGTTTCAACCCAGTATGAATCACCAGTTACTAAGTCAGGATCAGTG
TCTTTCTTTGGTAGTTGTGAAGCATCAGTTAAAACACCTTTGATGTTTAAACCTTCACCC
GGTTTACCTTGCGGACCTGCCATAATACCAACGCTAGTCCAGCGACTACCGTCCATGTTA
ACAAATACTTCGCCCGTGGACTTAACAGTATATGCTTTCCATTGGTTATCAGCAAGTGCC
TCTGGTGGGGTATCTTCAGGAGTGTCAATCGCTGCGATGATCTGGATAGGTTCACCAGCG
GGACCTTGTGGGCCTTCTGGGCCCTCGGGACCTTCTGGACCCTGATCACCGTCTTTACCA
GGTGCGCCATCTTCACCTTTAGGACCTCGATGTGTACCACTATTTACCCAATTACCATCT
ATCTTAACAAATAAGGTAGATGTGTCTTGTGTGAAGTACGCTGCCTGATCAGGAGCATCT
GCCGGTAGGGCTGCCTCATTGGCAACAGCACCAGTAATAACTACGTTCTTACCATGTAGA
CTGATTAACCATTCACCCTCAGTACCAGTGAACCCATTGTCCAATGCTACTTGGAAAGCC
GATTTACCTTGTGCCCCTGGTTCACCAGGTATACCTTGATCACCATCTTTACCTTGTGGG
CCTTCTGGGCCCTGCGGACCACGGAATGAACCCATGGCTGTCCATGAGATATCAACAGTC
GGATCTTGGCCAGTCAGTACATAAACCGTATTGGTGGAACGTACTGAAACACCTTGGTAT
TGTTCTACACCAGTAGTGGTTAATAGATCATCAGCAGTTTCAACTGCTTTAACTACTTCT
AAGTTACGACCTTGTGGACCAGGTTCACCTTGTTTACCATCGGGACCCGGATCACCTGTA
TTACCTGGTTTACCTTGTGGGCCCTCAGGACCTTGGAAAGTACCTAGGTCTGACCACGCT
CCTTTTACAAAGATGAATAGATGTTCTGGTTTGCCAGCTTCCATAGTAACTAAGTATGCT
TCTGATTCTTTAGCTACAGATGGATCAGGTAAATCAGCTGGTGATGGTAGTGAATCCTTA
ATGGTGAATGGCATTGCTGGTTCACCCTGAGGACCTTCAGGACCAATTTCACCGTCTTTA
CCTTGAGGACCCTCGGGGCCCTCAGGTCCACGTTCACCCTGTGGTGTAACTGGGAACCAA
TCATAGTCATCAATAGTTGGATTCTCTGGCGGATTATTACCTGAGATAAGCATCCACGCC
CAGAGTTGATTATTCCATGAATAAGCATCTCCAGGTTTATTACTTTCAGTCTTAGGTAAT
TCTAAGTTATTAGGCCAACGACCACGGATAACTAGACCAGTACCATCTGGACCACGAAGT
GAACCTGTTTCAACCCAACGACCTTTTACAGGGGGATCTTCGTTACCTAGCGGTGATTCC
CATACCCATAGCCGATCTTCAACGAACCAAGCATCACCGACATTATGCGTATCTAATGGT
TTAATTTCTTGTTCGAATGTGGCTAGATCAACATTAGCGATAATATTGATAGCTGGACCA
ACCGGGCCAGGAGGGCCTTGGTCGCCATCTTTACCCGGCTGACCGTCTTCACCCTTTAAT
CCAGGTATAGTAACAGGATCAACGAAAGTGGTATTAAGACCCACCCCGGTAGAAACCGGG
ATGTGTCCATTAACTACAGAAGTGGGTTCATCAATTGATGATACCCGTTTGGGGAGTTCC
CCGTTTACATAATCTTCAAATGCAGAAGCCATTATACTACTTCCTCAAGTTCGATTTTCA
TCAGTCCAGTGGTGTTGGATACAGCGTTTGCTGAGTCACAGTTATACCACTTATTGCCAT
CCTTGATGGTGTAGGTGTCAACTTGATTTGCATCATCCGCCTTATAAGTGAAGTTTAAGC
TACCAGTAGAACCTTTAGATAGGTTACTGCAACGTAGTTTACCAGTATTGGTTACTTCAG
TACCAATATCAGTTTCACGGTTAGGCCAAGCAGCTACACTTAGAGTGCGGAATACAAATC
CACCTAGTACGTAAGTACCACCATTTACACTATTAGCTTCAACACCAGCTAAACCTTTTA
GAACCACGCTGTTAAAATCGTAGGTTCCTTTAGGATCATCGTCATGTACTAGTAACGCAC
GTGTCCATACCTTACCAGTGGCATCAGATTTCCAATCCTCAGTAGCCCACTTACCGCCAG
GCGCATTCATGGCCGGAGCTGCACTTAGTTTCTGAGTAGAGGTTAAAGTAATGACATGAC
TTTGTACTTGGGTTCCAAAGTTACCGCCTGAACGAAGGCGAGCAGCAGGGTAGGTAACCG
TCGCAGTAGGTGCCGCATTTGCAATAGCTACAACAGCAGTAGCCGTAGCTACACTACCAT
TAGCTGCACGTTTAGCAGTAATTAATAGGTTATTAGTGCTGTCGTTATAAGTACCTGACT
TATAAGTAACTACCTTAGCAATTTCTGCTTCATTTGCACTGGTTATAGTGAGCTGATCAT
TTGGTGAGGAATAAACCACACTATCAGCATTAGATACTGTATTGGCAACAGTAGCAGTTT
CACCTACTTTAATAGCACTTTGACCAGACGGATAAGTTACTTGACCAATATTAACAACTG
GGCGAAGATCATTTAACTGCATGGTGTTAATTTTATCTTCGGAACCAGCATCACTGGAAG
TAAACCATTCTGACCATGACCCAGTTGATTTCTGAACACGTACACGGAAACCATATGCCT
TAGTAGTATTACCACGATCAGCTACGTAAAGACCAGTAACCTCTAGTTCTTTAGTAGGTG
CAACAGTACCACTACTTTCTTTCAGTGCACCAAAGTCAGCAATTTCGTACGCTACTATAT
TTTCATCAGTAACAACTTTAATTGCAACAAGGTCATCTTCTTTAAGTTCTGTTTGACCTA
ACGGATAACCGCCAGTAAATACAGCACTAGTAATATTAGCGGGAGTATCGGCTAATACCT
TAGTAGTAAATTTAGCCCCATCGGCGTGTTCTACAACAATATCAGCATAACCTTCAGTAG
TAGGTGCACCAAGGTCAATATTGGCAGTAGCAATCCACATAGGTGGATTAACGCCAGGAG
TTACAACTGCATCTACATTATTGATTTTAACGCTAGGTACATAACTAGAATGTCCAGTTA
TTGCATCAACGATAACCCGAACATTACTAGTAGTTGATAGACATGACTGTACGCTGTGTC
CATCAGGGGTACGCACAATACTACCTACGTTCTGACTGGGATCAGTTGCTTGTACATCTT
TGACGAATACACCGCCACTAATACCTTCATTTTTAGGAAGGTTCTTAAGGTCAACGGTTG
TTTCATCAACGCCTTCAACAATACGGATATTGCCGTCATTCTTGAATGCAATGTAATCAG
TTAAACTACCAATCTGGAATTCACCATTGTCATAAATACGAATTTCAGACTCAGGTAATT
CGGGTAATGCGCCTTGCGTAGTGGGTGCAGAAGCGTTCTTTAGATAAGAATAACCTTCTG
CTTCCATACGACCTTGTTCCATCATACGCTGAGGTACTTTATCACCAGCAGTTAAGTCGT
CTTGATTTAGACGCTTATCAAACAGGAAAGCAAAGTTATGGAAAGCAGCGTTGGATGATA
ATCCACCATTCTTACCAGTTACACGAAGTACATCTTTAACTGTATAACCTTGTTGATTTA
AGAGTGTCTGGAGTAGTTCAGCAGTCATGGTTGCATGCAGTGATGTTAACCATTCCTGTT
CAGTACCTTGGAAACCATTTTCCACAGCTACTTCATATGCACTCTTACCAGCATCACCTT
TAAGGGATGCTATAAACTCTTCTTCAGTACCAACGAAACCTAGATCGACTGCGATTTGAT
AAGAAGATTTACCTTTGAGCGAATCAAGCCATTCGGTTTCAGTTCCCTGGAAACCATTTT
TAACGGCAATCTCGTACGCACTAAGACCGGGTGCCCCAGTCTGTAGTTTTGACAGCAGAA
ACTTATAGTTATCGACTTGACCAGCTGCATCGGTATGAATTAATTCAATATACTCGCTAT
CGTTAAGAGTAGCTAGATCCGGCAGTTGACTGATCTTGCCTTTGTCTGTAGCATTTGCCA
TTTTTATTTCCTTGCTGTATAAGAAACACTAACATGTTAGTCTTTCGAAGAAGACTCATA
CAGTAATGTACTATTGCTAATGAGTAATATTTTAACAACATAAGAGAGTCCCGAAGGACT
CTCAAAAAGTTACATTGACACAAGATGTGGTTTATCATGAGGGGAAGTTTCACATGATAC
CATAGACTCCATTCTATTGCTACATCTTATTTTCTATGTGCTGCAACTTTACCATCCATT
AGGTTATAAACTTCTTGAATGGTTTTAGGTGACCCATCTTTGTTATAAAAAATAGATGGG
TTTGCCTGAGCTTCTTTTGGGAAATGGGTAGCCGCTAATTCATTCTGACCAGTGGTCAGG
AAACGACGGGCTGCACCAGGTCCAAAGAAGTGAGCTAAATAAAGATCAGTATCAGTTGGT
TCACGTTTAAGGACAGGACGAAGAATATTCATATTCTCTTTAATTAGTTCGGCACCCATT
AAAGCACTTATACGTGGATCTTTACGTAATGCACCTGTTGGATCAGTAATAACGCCATAC
TTCATGCCATAATTTTCAATCATTGTTTTCCATGTTCCAGTAAGGAATTGGAACCAACCA
GTAGCTGATGAAGTCTTAGCTTTTATTTCGTAATCGAATGCTGATTCAATAGAAGCAAAT
GTTAATAGCAACTGGCTACGAACACCAGTAGCATTTTCTACTGCATTCATAACTGGAGTT
GCAGCTGCACGTGATTTATTTGCAGTTGGCATAGGGATAGTTTTATAAGGAATAGGTGGG
GAATATTTACTGAATAATTCAGCCCAAGTATTCTTACCTACAATACCATCACTATCTAGA
CAATTATCTTTTTGAAATTTAACCACCTGATTAAAGGTGTTATTTCCAAAAATACCATCT
GGCTTTCCAACATCATAGCCACATAAATTTAAAAGTGTCTGGAGTTGACATACCTCATCA
CCCCTATCGCCTTTGCGTAATACTTTCATGATATAACCTCTACATATTAACCCAGTATGA
ACCCTGGGTTAGGTTCATTGAGGGTATTACTTAACTTTCTAGATAACGGAGTTGACCATT
TTCTAGATAACGTGCTGCACCATTCTCTAATAGTCGGATAGTATCTGACGTATCATTAAT
ACGTTGAATTAGTATATTACATTCACCATAGTAAGCATAACTACTCGACATAGCTACTAT
CGTGTATGGAACAATATCCGTAGTTGCATTGATTGGTGTTGTATCAAATGAAATATCATC
TGGTCCAAGCTTCCAACCTCGTGAACGGTTAATTTCTTCAGCAATTGCTTTAGGTGTTAA
AGTACCAACTATCTTAAGGATAGATCCTTCACCAATAGCACGTGGATCAGCAAAATCTAA
CCGACGGTAAGTTAACATGTACTTAATGCCAGTAGTAATGTCGATCATTTCAATAAATGT
ACGTCCAGGCTCAATCGTGGTTGGACTAAAAAACATATCTTCAAATGTAACTGTTTCTGG
TTTGAAGTTTATTTTTAAATCATCCCGCGCAAGTTTTACTAGCGCGTCCCTCGATGCCAT
GTCATACGGAAAGATATCCTGCATGTCAAACCTCTATAGTGGTAAAATGTTTAATCCACA
TAGCATTATAAAGCCTTCCTCTTGAAGAGGAAGGCTATTATAGTTTTTATTGCATAAGCA
TCTTTATTAATTCAAAGGTACCTTGCACAATAGTCTTTAACACATTGGCACTTTCTTCTG
ACATAGGTGCACCAGATAAAACTACCATAGTAGATACTGATGCCCCAATAAGAGTAACGC
CACCAGCTAATACCAATACCGCATTACGGTAAGAATCTTTAGCCTTCACTTCAGCAATTT
CCCGGCTGAGAATTTTATATTGAAGATCTGAATCGGTAATAGCATAAATTAGACTAAGTA
CTTCCTCACCAGTTATACTTAGATTTATACGTTGTTCCTTTTCCAATACACTATTCAAAT
AGGTACGTATCTTCTTTCCATAGTCTTTAGAAGAAGATAAAGGATTACCATCGATCTCAA
TCAGAACGTCCTCTAGGGTCATGACTATTCTCCAAATTACGTTGACGTTCGTTCCATTCA
CGCAATTTGTCTACTTGGATATTACATAGTCCAAGTGCATCAGTTTGTAAAAGCCAATGT
TCGGTCATAAGTAAGAAACGTGCTTCATTCTCATCAGTAACCCCAACAGCTTTATATCTA
TCTAATGCATTGATGATCTCATTAGCTGTAGGTGGGGCTTCCACATCACAAGGGAGAAGC
AAACTACTTGGGATTGCAACTGGTATTTTCACGGGAGGCGGCACAGTAGTGCTCGTACAT
CCGTTTAGCAATAGCATTAATAGACTTATTAATGCTATCTTTTTTTGTGTCAGTTTCATG
TTCAATCTCAGTAGTGGTGCTTTCCCCATTAGTTGTTTTAGCTTTAACTTCAGGCCATAC
AGGTGGAGTATATCCACGACCTGATAAACTAATGTACTCATCAATCACCTCAGTGCGTGA
TTGCGCCTGCTCTTTTTGTATCTCTTTCTTTTTAGTAATTAGTTCAGCTTGAATTTTATC
GGAGATACGGTCTGATTCAATCCTAACCTTTTCTGCCGCTTCATAAGATTTAATTTTTCC
AATTAAATCTGTAGAGCGAACAAGTTCCGTCTCATAACTATTTTTGTAATATATGATACC
AATTAAGGCACCTATCAGAATAACTAATAGTAACCATATATGCAATGGTACACTACCAAT
TATTTTTCGTATACCTGCTAATAGGAACATTGTTACCTCGAATTGTTAAACAATCCTAGT
AAGGTAGTAGTAAGCTATCGCACAAGCATCCATAGAGTGATCATCTAGACTATCTATGTC
GATGTTCCATGTGATGTCAGTACGAGAACGCATTGCTTCTTTAACATCGTCTTTAGTAGT
GCCCTTTACTTTTTTCATACCCGCATTGATTTTAACTGTGGTAGGATCAATCATGTACAA
GGGTATAAATCTATCATACTCGTATAATGCTCTATGTACATAGGTCACACATTCTACAAG
TACAGCGAAGCTTTGGGCAAACTTACCAAGATAGTTAGACTCTACAATAACTGCATGTGG
TTTTTCAGTATGTAGTATATTTAGCACCACATCGCTCATTTGCTGCATTCTAGTAAATCT
ACTACCATGAACCTCGGAGATGGCAGCATATGCCGGATTTGTATCTTTAAGGTGGCAAGT
AAATGCATTACGTACCACCTTATTACTTCCATCCAACTTATCTTCCAGAATAGCTATGCC
GAGGTTTTGTGAACCAGGGTCTAATGCTAGTACCCTAAATGGCTCATCACTCGCTGGAAG
CTTAAGCATATTTAATCCTTAGGTACAAATTTTGCTACGTTAATAGCTTTGCTACCTAAC
ATGGGTTCAACACCACCAATATCAAAGGTTAACTTCAGACCATCGCTATTATAACCAACA
GCATGGTTAGTAGCAATGAAGACGTTAATCTGACATGCAATGACTTCGGTATAAATAAAG
TTACCAGATGCACCAGATTGCCCTTGTACGTCTTTATCTACACCTGAACATAGTCCGATT
TCAGAAATGATAGGTGACCGAACAGAACCAGTCCTGATCCTGTGTGCTGTGGTAATCTCT
AAGATCTCTTCAGCTGTTAAATTAACAGTCACAATTGCAGATGCAGAAATTGATTCATTT
GAACCAACTACAGTGCCTTGGTTAGAAATCGTCGGAGGAGTTGGATTAAGATCATCGCGG
TTAGGCACGTATGGTGTTACAACTGGTTCACCATCAACGATAGTGATCTTTAATAGTTTA
GGCACAACACCAGTGACATCGATCTTACGTGCATAGTAGGCAAAATATTGTTTACCATTA
TGTTCCTCTAATCGACGTAACCGATACATATTACGACGTTGGGCTGGTAAGTCATCAGCT
ACTTCACGTAATACGAAAGGAATGTGATTATAGAGTGCAGCATCATTCGCTCGATGGATA
TTATTTTCTACTTCATCAGAGCCATCTGGTCCTTTAACAAAATAATGACCACGGTTGCCA
ATTACCATATACTGAACATTAGGAATCTGGCCAGCAGATGGTGCGACATTTTGTTGAATA
GAAAACTTTTCATTAAGAGTGGTATTCGGAATTAGTTTGAATGGAATACCTAGATATTTA
CACGTTTGCAGGTATGACCCATATGCCGTACGTGTAACGTTAATATACTGCTGATCAATT
GCAGACATTTTAGAATCCTTGCAGTAGATGGTTAAAATTTAGTACACTGTACTATTGAGT
AAATTTGTAAGAGTTCCCCTCCACATGGAGGGGATTTATGCTGTTAAGCAAATTTTTCAG
GAGCACCACGTACCATTACACAGATCCATGATTGTCCATTATGGGAGCGTTTTACTTCAT
GTAATTGGGATAGATGGAATACTAATCCTTCACCTTTTCCAATTAATAGTTCCTCACCAT
CTAAAATGAACTTAGCATCAGGCCCAATATTCCAAGCAAGAATAGTACCGGCCTCGGGTT
TATCTACCCAATAATTCATCTCATCTAGAAATCCATCTATTTTGGGGAAATCTAAAAACC
ATACTTGCAGTGCTTTGTTAAATAGTTCTTCGCCTAAAATTGGTTTAATTTCTTTACGCT
GTTTAGCAGTCCAATTAAACCATTTTGAAGTATTATAAACACTACATGCTTTAAGGCCAT
TGGTGCCAACTCGCATACGGTGGCCATATGAGTCTCTAAATGAAACTTCGCCTTTAAGCT
TTGTTAATGTTTTTAGTTCTGTCTTAGATAGAGTCTTAAGTTTAATTACCATGTTGTACC
CTACTCGTAGAGATATATAAGAACGTATCTTTTATTTTTAACTGGAGGGACTTCATGTGG
CTCTGAACGGATTGGAAAAATAGTAGCCCTACCTGGCACATCCTGATAAAACTTACCATT
GATCGTTATACCATCACCATTGTCATTTAATGCAATAACCATGTTGTAACGATAAACAGC
TTGGTCGATGTGTTCAGGCATACCTGTACCAATTTCATATCGGTTGATTAGAACTTCGCC
TAACCAAGCACCTTCTACCTTAGGTGCAATATGAAATAGTAAGTCCCGTAATTCCACTGG
CATTTGATTATGACCTAAAAACCAATAATCACATTCAGTAATACCTTTGGTTGGGATACT
ATTAATTTTTTTCCGATTAAACCACCAGGAATCTCCCAGTGTATCCAGATAAGATACAAG
GTCAGCTGCAAATTCAGCTGAAAACACGTCCTTGGTCGATCTTTCGTGGGCCGATAAGCT
CATGCAGGCCACCTGCATATCGATGTGCTTTCTCAGCGTCGAGATCTTCCCTGGATTTAT
AGATAACGTCTGGGCTCTCAATTAGGGCCTCCTTATGCGCGCGATTAGTTCGTTTAACAA
GATCAATATAATCAAGCATTTCTTCTAGTTCATCTAACTCTTGACCATATTTTTCAGCTA
GCTGTTTAATTGCACGTGCAAGGATATTAATTTGCTCAGTCACTGGATAGCGTTCAGTAA
TTTTGTAGGCCACTTTACTATTTAAGTGATCTTCAGTAATTACTTGTGGCGCTTCTTCTT
TAGGAACAATCTTCCAATCAGAAAGTGTAGGTTGTTTACCAGGTGTATTACTTACGACTA
CATCATAAGTACCCATTTCGGCTTCAACAAAAATGGTGTGCTCACGTGACTTAGCATCGA
ATGCATCTAAAGGTAGATAGCCTACGCTTTGTAGGAATACCTCAGAAGTACGGTTAAACA
TTAATAGTACTTTTACTTTATCTTCAGTAGGCGCAGTAGCTAAAGATAGTTCTTGGAGTG
ATAACGGATTATTCATTATACAAGTGCTCCAGCTCGAATCATCCAACGCCATGTACCATT
ATTTCTAACTGCTACATATTGCTGGTAATAATAGGCTTCAAAGGGGGTATTAGTTCTAAC
ACCTACAACAGCGTAAGTGCCTTCAGGATATTTGGTATTATCTGTAAACATCGTATTTAA
CGTGTTTAATGCTTCTGCTTCACTATTTACCTGACCTAAGGTAATGGCTTTAATCTTATC
TTTGTTCCACTCTTTAAAAGCTTCAACCCAGTCTTGCCAAGTACCATCGCTTGCTAGATA
ATATCTCTCAGGGTCATTTAATATTGATGCATTACCTTTACCTAATGCATTCCATTTAAA
TACGCCACTAGTTACTTCTTCAGCTGGAATATCTTTTCGAACATCATTCCAGAAATTCTG
ATATACCTTACCTGTTCCAGTAACTGGATAACCCTGACTATTAGTAGTAGCACCAATAGT
GGTCGCATTAGCAGCTTGTTCATTAATACCTAATAATGGAGTTATTTTAGAATCCATCAC
TGGTTTGGTATATGATCCAACTTGGTCAGCTGTGACAGCATGTGGGTTATTAAAGTTTTT
AATATGTGCTGCAAGTTTAGCCAACGTATCAATATTAAGAATACGATAAATACCACCAAT
TGGCATGTATTTATTATTTACGTTTCTATTAACTGACTCAGCTTCAGTAGCAAAACCCCA
GTTGGATATCTTATCAAGCGGTGGAATTAACTTGAATCGATCATCAGCATGCGGATTATT
ATAATCAGTTAAGTGTGCTGTAACTCGATCTCTATAATCGTCCACAGCGGTTTTTGCATC
AGTGAGATAAACTTTATAATAATCATCGGCAGCATTATCCATGGCACTTCTACCGACCCG
ATAAGCATGTGCTATTCGGTTTAATTCAGTTACCATTGTATCTGTGCCATAAATCTGCCA
ATATTCATGGCCATGAGGGGCAGGCGGATATGCATCGGGTTTACCAACAATATCATCCCA
AGTGAAACTTAATTCATCTAATTCAAGTTTATCAAAAACAGCTTTAAGTTCTAATGCTGA
AATACTAAAGAAACCACCAACTGCTCTATATGTAACTTGGACATTAGGACTAACATTAGG
ATCAGTTACTACAACAATGCCCATTACTTCTAAGCCAGTCATTTCAGATAACTGTGAATA
AAGATAGGTTGTTTTATATTGATCTTTGTTTAAAGATAAACCAGTCTTCTTATCACGTAG
ATTAAAGTCCTTAGTATAAAAACCACCATGTGTACACATAAGTACGCGATGTGTTTTATT
ATTGACTTGAATAAGTGTGTGATCCTCATCTACAATGTAATTAGATGCTAATTTAGCTTT
TAGATCTAATGGTAAAAAAGTAATCATTATGCCGGTTCCCCTATATTAGTCCACGTTGAT
CCAACTTTACGTAGGAATAGAGTATTCCTATAGGTAGTAGCTGTTGTACCATTGCCATGT
CCAATAACGAATTCTTGATCGTAGTGGCAAATAGCTAATGAACCAGGCCACCATTGTTCA
GGATCTGAGAAGTATGTGTTATGGAAAGCAATTGCTTCTTGAATAGATTTAAAGTTGGTG
TTATTCCAGTGGACCATCGTTCCTGTTTTTTGGAAGCGATTAAATATAAAACCGAAATGA
GTCCATTGACCATTAGGCATCAATGTCCAATCACGACCTGGCCAGGTTGCTGAATTACCA
ATCCTACCAGGTGCTAATCTACCAGTAGTTAATACACTTAGAGGCATCTTAGCACGTACT
GTTTCACCCATGCTATAAACATCTTGTCCAAATAATAGCTTAGATGTATTAGCTGTTTTA
CCTTTATCTAATTTTTGATTTAGTAAAATATTAGCTTGAGTAACAGTATACGTATTTAAT
TGTTCAGCCGTTACCCTGTGTGGGTTAGTGGTAACATCGTTAACGTGTGCCAGATATTGA
ACTGTATAATTGGTATCAACTGATAACTTCATTGACCATGGCGTAGCATAGACATTCATG
ATAGCGCCATTTGGCTGTTTGGCTTGTGTTTCAGTTGCAATACCAGCATTATAAACATTA
CCTAATGCCACTTGCCCTTTACGTGTCTGGTGTGGGTTAGTGGTGGTATCATTGATGTGT
GTAGTAAGTCGTGCTTCAATTGTATTAAGATCAGCTTCTATTAGACTTAACTGATATTTA
AACTCATCTAAAATGGAGTCATATGAGTCTGATGAGCTTTTCTTTAATAAGTCAGTTATT
TCTTTTAATTTAACAACTCTAGGCGTAAAACCATAAAGTTCCCAATAGGCATGCATATGT
CCATTAGGTTTAAATTGGTCAGGAATATCTAAAATATTAGACCAATGTGGTTTACGGGTA
TTATTTAAAAGAGCGTTAGCATGCTCAGATATGGAAGGGGCAAGGTTACAGTATATACCA
CCGCACATCTGAGCATCAATGTAAATATCCTCGTGGACATCTTGTTTTGTAATAACAATT
ACTGAACAAGCTTCAAGTCCAGTCTTCTCAATTACTTCTGGATTAATACCAGTACACTGA
TAATCCATTTCTGGATTTAACTCATTTTGCCTACCATCTCTGATAACCATTCCTTTAGTG
TAAAAGTAACCCTTATCCATCACGATGATGCGATAGGGGAATCCTTTCTGTTTTGCTAGT
GAATGGTATTCACCCTTTGTTCGGTTACTATAACTCTCTCCAGTTTCATCAAATGGAAGA
GGTAAAAGTAATGGATTCATCTAAACTATCCTTATGGAATTACCCAGCCACCTTGTGAAC
GGAAACACACGTAGTTAGGAGTATAATCAACGAAAATCAATCCGTTACCAGCTGCCTGGA
TTTTCTGTTCTTTAATAGTGAAGAATACAGTAGATCCGATAGGCCATGCATCTGGATTAG
TGAAGTTAGTTCTACAGAAATCTAATGCTGCTTGCACGGATGGTTGTAATCCTAGTGCAT
AAAACTTTAACTTAACCGGATATGAATACTCTGTGGCTAATTGATCAAAGTGTGTAAAGA
TACCATTCCCACGGAGTGGTGCTTTCCATTGTCCCATTGTCCCGGAACCAATCCGATTTA
TATTGATGACATTACTCTGGCCATCGCCAGCTGTAAAGTTCACGTCTGTGTCAAAGTTCT
TACGCATCCACCAAATAAGATCGCTATAACCCATTAAAGTAGCAGTTGGTTGCCCTTTAG
GTTTATAATAAACCATATTAGTATTAGCTACTGTATCAGTCTTTAAATATTTACCAGTAA
TTCGTGAATCAAAATCACTTTTCAAATATGACTGTACATGTGTATTTTTGACTTTATGTG
GATTATCACGGCGAGCTATGTGGGCATTAAGTGGTTTTATAGCTATTTCATCTAATACGC
CATATGCTAACTGCGGTGTTAGATAATGCGCATTAGATTGACCTAGTTGAGCAATGGCTG
GAGTAGCCAATGGAAAGTTATTAACTAAACCTAGTTCAACATTGGTTGGGGTAATCTTGT
GTGGATTAGCTTTATCTTGAATATGCTTTTCTAGGCGGTCATTAAATTTCTTTAAGAACT
CATCATAAATCTCTTTAACTTTACTACGGTAGTCATCTTCACCACTTGTGTCACCAGCCA
TGAGTGCCTGTGTTATCCACTCCATCTCGTTGTTATAAGGCTGATACGTATCAAGATGCC
ATCGTTCGCCAACTAACTCACCGTCTTTCCAATATGGTTCGTTACCAGTGTAGTCGAGAT
CTTTTGGCTTTGGCTTACCATTTGTTTTCCACCAGTTAATATAATCGTCGATAACTGTGA
AACTAAACGCTAGATCACCGCCTACCATGTGAGCTGAAGTATACACTATTGGTGTACTAG
GTGTTTTTAAGAAAACAATTGCTGAACATATTTCTTGACCGACATACGCACTAGCATCTT
TGTGATAATAAGTAACAATATAATCAATATCTTTAACTAAACGATTATATTTACCATCAT
AAACTTCTAACCCTTTTGTATAGAATCCACCATGATATAACTTGATGATTCTATAGTCGA
ATCCCTGATACTGAACTAGAGTATGTGGTTCGCTACCAATGAAACAATCTGGATTTGTAC
CTAATGGATCAAATGGTAATTGTAAAATAGGATTATACATAAGAAATACCTTTTAACTGA
AACAGCAAAAAGAGACACAGCTTTTGGCTGTGTCTCTTATGCCGGGTCAGATGTTTATAG
ACGGTCTGCTGCGTCATTAAATAATTTCGCAAGTGGACCGTATATGTGCACTCGATTATA
ATCATCTGTCCGTTTAGGAGTCATGTACGTAGTGTTATTGAAACCTTCTTGTGCACGCTT
CTTAGTAGCTGGACGATAGTTAGCCACATTACCAAGACCAATATCCGATGGTTGCAGATT
ATGTGGGTTACCTCTAGCTGCAATATGCCCATTGATCCTATTAATAACGGCTATGAATTT
ATTAAGGACTGCCTTAGTAAATAAGCCTTGATTACTAATAGAAGCATTTATTTGAGCATC
CATCTCTTGACGTGTAACTTCTGCCATTTTTAAATCCTCTTATGGACCAGTGGGTGGAGT
TGGTTCAATATGTTTCACACGTGTACCCCTGCCATTCCATAATAAGGAGTTATCAGGTGA
ATCTCCCCAGAACATGACTTGATTCAAAGTATTTACTGTCACAGTGAAATTATGCCCTGC
TGCCAATTGAATATATGGTCCAGCATGGTCATCTACGTCACATTGTCCATTATCGTTATT
TCCGAAGAAAGCGATTTGGCCATCACCATACATCACTGTGTAATGTGTGTACAATCCTGA
TACACCACCAATTGGGTTACTATTTACATTAGCAGTTACATCGTTACCACGTCCATCAAA
CCATAATGGCGAGATAACCATAAAGGATGTATCATTACCATTGCGGATGATTTCCATGAC
ATGTACTTTACTAGATTCAGATGTATAATCTTCAGGAGCAACTAATGGATCTATTGAATG
CATGATGATGTGGGTGCAACCAATTGACATTGAATCAATAACATGATTCAATGGTTTAAT
TGTGTTGTTAACAAAATCATTAAAAGCACTAATGAAGCTTGGGATACCATAAGCTGTATA
TACATAGTCAGCACTGCTAGCTGGACCTATGTTTACAACAACTGTCATTTCACCAGGATA
GGTGTCTTCAGTATTGGTAAATATAGCCCATGGTTTATTAGCAGCAGGTACTAATAGTTC
AGATGTACCACCTTTAACACAACGATAAAGATCTTGGTGCTCAAAGATTGCTGATTCACC
TGTTGAACTAAAGATGAACTTACCTGTTAACATATACAGAGATGTTTTATTCGCCCAGAT
ACTTGAAGGAATATTTCCATCTATAGGTGCAGTCCATTCTACTGGAATTGGTATCATCTC
ATCGACTTTAGTTTCATCGATACATGCTGACTTGGCTACACCATTCTCTACCCAATACTG
ACCATATCGGTGAGATACCCAGGATTCCTGAGCATATCCTAAACGCTCTGGTATAACAGC
ATAAGGGTTAGCTGGCGATTTAGGATGAACTACACAGTCATCTCTGACAATTGTACAATC
CCACCAACTATTAAATCCAGCATAAGCATGGAAACCTGCCGTGACACCTTGAATTAATTC
CATTCGGTCATATTCACTAGTATCATCCACAATAACTGGTAATGTATCTATCTTTAATTG
TACATCATCCCAGTTAGCGTTATAGCCGGCTGCCATCCTTTCAATATCACCGAATGATGG
GAATTCATCTTTCCATTGCTCAGGTGTTAATCCACCAAAGGTAGAAGCATCACCACCACC
TAGTCCTGCCAGTAGTTGATCAACTTGTGTTTTAGTATATGCATTAACCTGGTCGGCAGT
TACAACGTGTGGGTTATTTTCATTACTAATATGTTGATTAAGTTTCTCTTCTAGTTCATT
AATTCTAGAAGCATCACCAATCTGACTATAGAGAATAGATGCAGTATAAGGTGTTAATAA
TACCGAATCATTAGTTACGTCTACCGCAGTAGTTGGTGTTGCAGCTGGGTAGTTAGGAAC
ATTACCTAAATTAATATCAGCTGCTGTTAGACCGTGTACGTTACCCCTAGCCGCTATGTG
GGCATTAAGCGGATTTACGGCAAACTTATTAACTAATGCTAATGTACCACGTGGGTTCGT
AAATAGATCATTACGATTAGCATCTAAATGCTGTTGATCACTTGCCATTGCATAGTTTGG
AACTAGTGACAAGCCAACTTGTGCTTTAGTTACCCTGTGTGGGTTATTATAATCATGAAT
ATGTTGGTCAGTTGTACCGGCAGCCGCAGCACGGATAGCTTCTATAATCCCTTCGATACC
TTCAAGAATCTCTTTACTACCTACTAATGAATCAATAGGTTGATTGTGTTCTAAAGGTGG
GAAAGAAGCAGGTAATGGACCAATTGCACCCCATGACCGAATCAAGGGGTTATACTGTTT
GCGTGCAAGCTCAGCCAGGATCTGCTGATTACTAAATCCCCATTGCCCACCTATTGTACG
ATAATCCATACCAATCATAGCGTTTATCGAACGCTTCATGATACGTATAGATCCAGCGAT
AGGTCTACCTATCGATTCCATAGCTTCAATAAACCAATGCCCAATAAGGTAATCAACACC
TTCTACATAAGTCTGATTGGTCAGTGGGTTAAATAGTCGTAATGAATCTACAAAGAAAGG
TGCAGCAAATGGTATAATGAAATAATAATCATCACCATCTGGTACCTGGAGAGTTTGGCG
CTCACCTTTAATAAAGTTCTGGGGATTTGTTCCATTGGGGTCGAATGGATATAACCCAGT
TTCACCGTGGGCCATGGTTGCCCTCCTTTAATAAAACGCTATTGCGCAATTTGTTAGCGT
AATGATGTGAAATTCACATAGGATTGTCCAATCATTCCATGGAGACTAATAGATGTACAC
ACTAGTACGTTCTCGCCACAGAATAGATCGTAAGTCGGGGCGCTGGACTGATGTTGATCT
AGCTAATGAAAAGATATCTACCTTATCTGTAAAGTTTGGAGATATTTATCTATATATCGA
ATATCCTGGGCCAACTGCCCCTATATTAAATGCTTTACATTGGATAAATGTTACTAATGA
GATTAGTTCAGCTAATCCAGATCTAACCGTACAAGAATGGTTAACATCTCTTGGAGATAA
ATCACTACCATTTGATGATGAACTACCCAATGAAAAAGTACGTTTAGTAAAATATGCACA
AGCATGGCATGCGGGTTATATTGCGCAGCCTGTTGCTAGAAACGGACATGTCACGGATGG
TGGTTCTAAGTTTAAGAAAGAAGATATTCTTTTAACCCATCCTAAACATAAACCTGAAGA
TGTTTGTCGCAAGTGCCTTATAACTGTAAATACAATGTTTCATCTAGCCGATTACACTAG
TGCTGGTGTAAGAATCATTGATGGAAACAAAACAGTTAGAACAGCTAATGACAATCAAAT
TGGATTATATTCATTTGAAACAATAGGTGATATTAAATATGTCCCTATTAAAACTGATAT
GATTTATCCGCAGCAGGTTGGAGCACCGCTATTTGATGCTACCTATATTGATATGCCTGA
AGATATAGACCTAACTAATAAGACTATCTTATTATCGCTAGGTGGTTATCTATGCGTATT
AGGTAAAGGTTTCAATAGGGTAGGTGTACGTACTTGGAGGGTAGACTTTGGTAGTCTATC
TATGTTCGACCGATACATGCAGTCACGTGAACTAATTGACCTAAGTTCATTAGGTTTAGA
AATTGATGACAAAAATAAAACTCTATTAAGTGTAGAACAAATTAAGAAAGATTCTGTTAT
CAAAGCTTACATGACTTTAAGTCAAACATTTTTCATCGTATTAGATAGCCCCTCTTTATT
CCAAGAATTTGTACCACTTGAACCATTAAAACTTCCTGGCAGATATCAGGCAACTAATAC
ACGTACTGAAGTTTATCCAAAATATACAAAACCTGATCAACTACCAGTAGTTGGCGCACA
TGGTAAGATGTTAGATTATCACTTCATACACGAAGATAAACATTCTGTTTATTGCTGCAC
ATTAAATATTAGAAATAATTTCGATGCGCACACACATGATTGGAAGACTGAGAAAGTAGT
TGATGGTGGAAGATATCCGGCTTGGCCTTTTGTACATGACACCGCATATCTTAGAATATT
AGGTGTAGATGGTTAACGTCATAAGCCCTCCCGTAATGGGAGGGCATTATGTTGTTTAGA
AATTATCCAATCTCATCTGATTCAGTTGACTCGTCTTCTAGCTCCTCAGCCTCAGCAGCG
GCTGCCGGGTCAGATAGTCCTTCCATATCAACTTCATCCTCATCTGTTGGTGTTAATTCA
GATTCAGATAAATCATCATAGCCAGCTTGTGTATCGAAGCCAGCTTCCATTTTATTACCA
CCACTACCTAAATCATCGGATGAGTCATCCATATCACTACCGAAGTCACCATAACCACCG
CTGCCACCGCCAGTTAATTCATCCATATCACTCCGATATTTCTTATATTCAGCATCTAGT
CTTTTCTTAATCTCGGCTTCACCTTTAATGAATTCCAAGAATGCTGAACCTTGAGTTTTC
TGAATGTTATTCAGCCAATCAAGTAAGCTAAAGGCTGGCTTATCATCGCCGTCTAATTCT
TCTAGAACTTTAGTTTCAGGAAGAATATTCTGTTCAGCGATAAACTGACGTAGGAAGTAT
GATTTAGCAGCAGCTAGATATTTCTCGGCTGCGCCAGATACACCTAACATTTCATCAGGG
AATAGTTCTGGAGTAATCTTAGCTGAAAGAACGAGATCTAGTAACCTAGAGTATTCCTCC
AAGGCATCAATCTGACGAGTATGTTGGGTATTATCAGGAGCAGGAAGTGTAACGTAAATT
GATTCAATAAATTCATCAATCACTTCATCTACTGAAAGATCTTTAAATACTGGTGTATCA
AGCATGCCAACATTCGCTGAAATAATTTCAACCATTTCTTTACGGATGATAGAACTATTG
GCTGAGAATATTTTCACTAGCTTAGTGATTTTATTACAAAACTCACGCTGATACTCACGA
ACCCTGCGGGAGAATACTAGGTCATTTTGCACAAGTCCTGTAGCAAAATCAGGAGATGTC
ATTGGGTCAACCTTCTCAGGTGGTACACCCATAGATGAAATGTGCATTCTCCTTAAACGA
TCTTGGAGTTCGGGGTTACCTGCTTGGACATTAGTGTTGTAATCATCGAAATCAACAGCT
GTTGAAGCATACCCTGGATTATCACCTGGATCAATTTTAAAGTCATAGCCAGATCGCATC
AATGAATCCATTGCTTGGGATGGATCAGGTGAAGCAAGTGGGAAACCACGATGTGCCATT
TCCATAATACTAGACTGAACATGTTGTACAGTGTCTTCTGGATCTGGGTCATCTGGATCA
AGATGGATAGTAACGCGTTTACGTCCGACTGCGTTACGCATGCCACCCATTACATCGGCC
ATGAGTAGAGTTGAACGCATAGTAGCCAGTAGCTTAGACCGTTCTAATTGAGTTTCACCA
ATACCATGTCGATTATAATTGAATGCAACATAGACCATCATCTCAGCTGGAATGAATACT
AATTGAGTCTGCTTACCTTTCCAAGAACGATAAAGCATGATACGCTTAATTTCTTCATCT
AGTCCAACTTCTAGCTCTTCATCATACATTCCATTGCGTAAACGGTTCTGTAATTCGTTT
ACGATGATACCTGCATAAGCATCATTGATTTGATGTTGTTCTAATTCAGTATAGTTAGAT
TTATTAATACCTAGTGCATCACGTGTTAGACGTAGTATTTCAGAACTATTGTCTTGTGAA
CGACCATTCTGCCATCCTGACTGTAGTTCACCATAGAAATCACGTGTAGCTTCTTTACAT
ACGGGGTATCCATTTAAATCCACTAGTAAGAAATATCCAACATGTTCATAAGGCCTGCCA
GGTACATATACTGGAATAACTGCTTCAGATGGCAATGGTAGAATTAGCGGATGACCAACT
GAAGAACGACCCATGAATTTTGGAGATGAAATAACTTGCGTATATTCAACACCACCACGT
GATTTTTGATAAAGTTCATTTATCTGATCATTTGTGAAATGGTGTGTTTCAGTTGCAATC
GATACTAGTGATGTATTGTATTTGCGTAATTTATTAGTAATAAGTTTTCGCCTAATACGG
TTAGATAAATGTGGTGCTTTCAATACATCGATGTTATCAGTCACTGATAATAATTTATCG
CCGCAGATATAATTAGAATTATCATTGTTTAGATTATGTCCTTCCATTGATATTCTTGAA
TTAGTAGGATGTCCAAGAATACCTAATGGCCTACCCTTATTAAGGTTATTGCGAACCATA
GCATAACTTTCCATGCTAACTTCACGTCGCCCATTAACAAGTGCATCTAAAACGTTTTCC
GGTAGAACAGCTAATACAGATGATCCTTTATGATACAGGATATCTCGTAACATGAGATCT
AGCCGTTCGTTAATCTTATAGTCTTGTTTGAAATATCGCTCAACTGGCTCGATTAGTAAT
CGACCAATCTCACTATCAAAAATATCTTGGTCACAGTAAAAAGTTAGATCAGCTTCAGCT
AGATCTTTAGGTGATAAAATACTACCAACTAACACTGTCTCAGTTAATTCTAGATCAGGT
AGCACCTGCATAATAGAATCTGAGTCAATAATATTCTGTGAAGTTTCACGAGATAATTTT
TGTAATTGATGTTGATCCACAACAATACGTGGGTTATTGGTCGAAGCCCCAGCTTCTTTT
GGATCATGACGGAGTTTGGCAATCGTAGCTGCCAGTGGTGTTGGGTATTGCCCACCTACT
TGCAAATTAGCGAGAGGTTCGTAACGCCCCTCATTAACCCCTGGTATAGCCATTTGAGGC
TCCTAAATTTTAAAGTGAGTAAATACCAATGAATAACAGCAACAGTGTGTATTTCAAGGT
ATACCTTGATGACGTTATCCGATTGGCTCGTTCAATAGTCATAAAATTTGATGATATTGC
CGATCAAATAAATACAGAGCTTTATCGAGATTATTTATTTAAAGCAGATCCTGATAAACC
ATGGACCTGGAAATACTATCTGAATCTTGCTGGTGAGTACCATGAAACTGATACGATGAT
GTATGTCACATCTGCTGACACATTAGAACGCATCGAGTTCACCAAGGAAAACCTCAGGCT
ACATCGTGCAACTGCACGGGAATTTACACCAGGTAGTCAGTCTTATAATAACTTAGTATT
AAAATATCCAACGCAAGCTGGACTAATCAATGGGATACTATATCCAATTGATATCAATTT
TGCAATAGATTCAGATAATGGGGATATTTTGTATTTTGATAACAAGTATGTTGAAGGGAA
TGAAGATAACTTTATTCCTGAACTACAAAGTTGGATCAAAGCATTTTATAACTCTAACTA
TAATAGCCAGTACCTTATTACAGATGATCTTTATTTAACTGCTTTCCTAGGGGAACTAAC
AGTATTACTTCCTCTTGAGATCATGAACATCAGACTAGGTAATGCAAGGTCTAGACGAGT
ACATAGTTTCCATATTCGTGAATATCTAGCATCAGCTAATCGATTAGATGAGTTCATGCC
ATATCTGAATAAATCTCAACAATTATGGTTATACAGGAATATACAGTTCTTACGTAGAAA
CTCAGGTAAGCAAGCAATATTTGACCAGTTGGTTGAAAAGATATTAACTGAACGTGGAAT
ACCAGTTATTAGTTATACTCTTGAACAAAATACTGCAAACATGCCGCCTAATCTGAAACC
AGATGTAGAGATGGTTAAGCATGACATGAATATACCTGTTGTGCAACCTGGTCAAGATAA
AAACACAGTATTAGAAATATTAACTAGGGAAGACCCAATTGCACGTGATAACCCACTAGT
TAAATATGACGCCGAAAAAGAAATAACTGAACAAGTATCTACTAGTGCTTTTAGCAATCT
ACCTACGCGTGTACTAGATTCAGAGGTTATCGATAGATCTAATAGCTCTGTAAGAAATCT
AATGAATGTCCTACTTAATAACTGGTTACATTTATCAACCAGTGGTCGTTATCGTGCATA
TGTATCCATTCCTAATCCAAGGACTGGTGAATACATGTTAATGACGGTTAAGGATGCATT
CATTGTAATGCTTTATTCTTACGGTAGAACAAAGGATTTTAAATTCGATAAAATACCTAG
TGTACCTGCTTATGAAGTATTACGTAATCCATTCCCGACATATGAAGAACTTCAGGTACA
AGTTGATAAGAAACTGGTAGGTCCTAATCTACTAGGTGCAGTCAGAGATAACTTTACCCC
AATGGGCGATTATATCTCAACTGAACTCTTCTATACTGATAGCTCTAGATTTCACAAAGA
ATATTTGAAAGGTTGGGAATTATATTCATTTAATGATCATAAAGATATTCGTTGTTATCT
AGAACAATCTGTTAAGACTTTCTATATTAACCGAATGTGTAAACTCGTTAATGAAGATAT
TTCTTTTGAACAGTACTTCAAAGATTCTGCTTTTGCTATTGCTGATTTAACAACTTCTGA
ATATGAGCAATTGGTAGTTGATTGTATTAATATTGCGACAGGCTCTGACCTTAATCGTGT
TATTACATTAGGTGAAATTCAAAGAGAGCTGTTAAGATTAGTTTCTAAACTATCATCTTA
TCCATTACAGTTTCTACGTAATGTTAGTTTTACGGATTTCCATGTACTTGGTATTGTAAT
GCCTAGACTTGGTGATTATGGAGCTGCTGGTAATGCCAACCATATTGTACCAATTAATAA
TATCAATGTCCAAACATATCGTACATCAGCTGAAATGACGTACTACATTAATGACGTAGA
TATTATTCCATTTAATGATTGGTCTTATTGGGCTGAAGACAGATGGTGGATTAATCCATT
TGTTGGTATTAAAGAATTAAGCCTATCTGAATACACCTATCAGATTCCAAGTATGGTTAA
CGTATGGCAATATACTGAAGAAGTATCTGAACACACACCACCTGATGGCGGTCTTGGACA
GTATAATAATTCAACAGATCCTAATTGGCCTAACTTACCTTAAGGAGTGATAGATGTTTA
TAACTAGGGATTACTGTAAGGTTGATCCTTGGACGGCTATTGTTGAAATGATTAATAATA
ATTACATGTACCAGTTAACTCCTGGTACATGTAAGCTATTAGAATTTGAAGCTATTGATC
ATAAACGAACACGTATCAAGATAGATCCATGCAGAGATAATAAGCCAGGTGAATTACTAC
CTGTAGTTGAACGTACTGAATTCTTTTATCAACGCTTAGACTTTAATGAGTTTTTTAAAG
TTCCGCAAGTAGTAGAAGTAAAAGATGTATATCTACCTTTTACAACACTAGATCTAGCAA
AACTAATCTGTGAAAAGAATGATATAAAATTTGATTTAGATGATTTCTATCATCAAGAAA
TAAATTCTTATAATCAGGATGTGACTATCACATGTAATCCAGATTCATTAAGGTTTGTGG
GTGAATTTAAAATAAAGCTTATAAATACAGTTAGGCAAGATATCTCTTTATTAACTAAAG
TAAATTCATTCCCAACTATTGTTAAATGGCCAGCCGGAAATGGTAATAAACCAACTGCTG
AATTCATTACCATGTCTTTAGACTTTACAGATTATCGTAGCTTTCTATTACCGATTAAAG
CATTCAAAGAAAATCCATATTCTTTAGAAATGGCAGCCATCTTAAAGAAAGTAACTGGTT
TAGATTTTGTTGCTAAAAGTAGTGCTACTAATTACAACATTACTAACATCGTTAGAAATG
GGGTAAATAACTTCATAGTGGATTATAATGGACCAGTGGTACCAGCATATAGCCTTAGGA
ACGATATCACTAATGTGATGGTTTTAAGGCTGTCTAAAACCATGTGTCCTAATATCAATG
GCCGTTTATTAATTCACTATAACTGAGGTGTGTAATGGCTGATACAAGAGCTGAATTGGT
AGCTGCTTGGAATGTGGCTAATAAACCTAAAGATAATAATGGTAGGCCTAGGGATTTAAC
TGTTGATGATGTTGAATTCATAGGAACTGATACATTCTTAGCAACTGGTACAAATGCCAA
AACAACAATGAGAGCGAAGACCAGTTCACTTTATTTTAAAGGTGAGGCAGTTATTTACTT
TAATAGACGCTCTTTACCAGAGTACTGTCTTGGCATAAAGGTACCAGGTAAACCAACTGA
CTATACTGATAATTTAGAAGTAGCTGAAAAATTAAGGGATGTTTACGGTATTCCATTTAT
TATAGAAGAGATACAACCGCAGCCTATTACTGGGAATACGGTTACGATACATCCATACTT
TAATAACTTGTCATGGATACCAACTATACCAGTAACTCTTGAATTTGAAAGCAGTTAAAT
AAAAAAATAAAGACATAGCCCCTCTCCATGCGGAGAGGGGTTTATGCCATTACTTAATTA
TCACCATTCAACCTTAAATTCACCGCATTTAAATGACAGTTTATTAGATCGATGATCTCG
CCCAAACCAACATCCTTCCTTGGTGACTGCATCTGCAATTTGTGGTTTAACCATGTTGTT
TTTGTATGATTCACGAAGTGCAGTATATAGGGCTTTATGACATGTTTCGAAGAATACCTT
ATTAATAGGATTCTTATCGTTAACTCTTGCTAGTGAATGTTTATCAAGCAGAGTGTGCAT
TCCAATTGAATACTTCTGGATAGTTTCTAAATCTTTCTTAAGTGCCCAGATACCATAGGT
TACTTTTAGAGAGGTATTGGCTTTGGTTGATGGAATAAATGCTGATGGGTCATCAATCAT
TGGTGCTAATTGCTCAGTAGAATATGGATTATAAGAAAGATATTGTTGACCATCCCAAAT
ATACATATCCATTGTATCTTGAACGACATATAGAACTAGGACTTTACCATTTGCCGGTAG
ATCACATTTTGTATTTACTTCTACAGTGTCATTGAAATCTGGTTTATAGTTATTATAACT
ATTCATCAATACCACATCATCGAATAGCCCTGCCATATTCACCGTGTTTTTGTTTAGTAA
TTCTTTGAAGAATTTATTCATTTTAGCTAGCATTGTATTACTCCTTTTTTACAATATTTG
ATTAAACAACACATCATTCATAAATAAGCCCCTCCGAAGAGGGGCTTTTATGCTTATTTG
ATAAGCTTAAGTAGAACAGCATTATCAACAAGAGCACCACGAATAACTTGTAGATAAGAT
CCATCAGCAGTGATAATGTACATTTTGTGTGTACGTGTATCATCTGAATCTTTAGAGCTA
TTTAGACGTTTTGCCATTCCCATGATTACTTGAGTAATCTTAGGTTCGGTAAGTTTACTG
ATAAGTACTGCTTTCTCAGTGTAAATATTTAAGCAGGATAGTTCTTCAAGACGCCAGCCA
GTTTGGAAACGCACTGATAGATCTAGAATAGAATTACCTTCTTCTGGATCAATATTAACA
CACATACTCCGCCCAACAATAAACGGAATACTAGCAATAAATTTATCAGCTACTGACTTA
CCCTTCTTACCGATCAGATATGCGATAAGATCACCGATATCCTCACAGAAGTCATCAATA
GTTACCTTCTGACTCATTGAATCAGCCAGGAACTCATTAACTGCACTGGTTAGCCTTTCA
ACTAGGAACCGATAGTAGCGTTGAGTCAGCTTACCATCTAGCATTAGTTGCTTAAGGCGC
TTTGCAATTAGATGCGGATCGGGTTCTTTTCGAATATCCTCGATCTCATACATAACTTCA
GTTGAAGCATCAAATGGATGGGACGACGCTGCACGATACTCATGTGCCGGAACTACATCA
TCTTCACTTAGCTCAAGTGCTTCACGAACTGCATTGAGTGCGCCAGCTTCTGCTTCAAGA
TTATTTGCACAGGCAATATAACCTTCTAGGATAACTGGATCATGTTCACGATTATCAGTT
AACCCGGCATCATTTGGTGAAAACTCTTCGGCTACTTCTTTAACCGGTTTGGCATCACCC
ATAATGGTAGATAGCTTATAACTGGTTGCAGTACGTGCGCCACCACCACGATGTGCTTGA
CGACGGAGATCATCTTGAAGCTCATGCTTTAGATAATCCATTTCCATGTGATTCTCCCAC
TTACGATATACTTCATGTACAGTGTTATCAGGCCATTTAGCAAGGAAGCGACAATATACT
GCCGGGTCCATTAGGATGCTGTATGGATTAGCTGAACTAGCCGTACGTACCCAATCAGGT
TTATCTGCAACTAGATGAGCAGGTCGGATTTCAATACCGCCCGGATTATAGATAACATCC
ATCGGACGATCTTTATTAATTACAAATCCGTTGGGTACATAATAGTTCGGATCGAATACG
AGTTCTTTCACATCTTGAGGGATGGGTAGATCCGTATTATCATTAGTTACAGTATTTGCA
TTTTGTTGTAGTTCAGGTAGAATCATTACATCACCTCCACCAGGGATTGTATCATAACTG
CTATTTGGAGATACTTCAACAGTTGCACCCCAAGAGTCTTTAGGTTGCATGACTGGTTTA
TTTTCCAGGAAAGGAGAAGAACCAGGTACGTCATATGCAGCATTTGCAATTGATGAACCA
GTGTCATGATTAGTTGTTGGTGTATATGCCCCTGGAATGTTCAGCTGTGGATTAGCCATG
GTAGCCGGTTGACCATAGGTCTGTTGGATACCTGCATTAATTGGAGGTAGTTGTGAACTC
ATGCCAGGACGACCCATTGCTGCACCCATCCCAGGGAAACCTCCAGGCTGTACACCAGGT
TGTTGATATCCACCCTGCATCATGGTGTTGCCACCCATACGCATCTTTTGGATATCTGAT
TCAATCTGTCCGATTACACCAAGTGCCTGTTGGATATTCGGCATTAGATGTTGAACTAGA
CTTTGTAACTGCGGGAAGGTTTGAGCACATTTACCAACAAAACCAGACCATACTAGAGAT
GCAGCTTGCTGCATTGCCTGCTGTGGGTTGTTCACCATTTGACGATGAATAAATAGAAAT
TCTAGAAAGTCAATAACTTTCTGAGCCCACTGGTGATAATAGTTATTCTGGAAACCATTA
CTAGATAGCAGATTATACGCAAATGCATGAAGCTGAGTGGTGGTAGCCGCATTTTGAGCA
TTCAGCCGGAACAGTCCAATTACACCATTGAAATATGGTTGTAGTTGAGGATGTTGTTGA
TGTGGTGATAACTGTAGCGGTAAGCTAAACAGATAATTCCCATTAGGTAGGTTAGGATCG
AAAGTACTGGTATCTACACTCTCAAAATCGACAGGTAGATTGTACTGTGCATAGTTCATG
TGAATAATACCTATTAAGTAGTTAAAGATTTAATTTATTTTGCACGGAACATTAATTGAG
CATTATCAATTATTTCCCGTACTTCTTCACGACGTTCAACTAGTCCAGTTGGACCCACTT
TGGTATACATATTAAGCCTACCTCGACCATCCGGGTTGTTCTTAGGTTGGTTTTTATATT
GACCTACCTCAGCAATAGATGCGTGAATAATCCTAGATGAGTCATTGATTAGAGACTTAT
TATGTGCCTTAGCTGTTTTAGCTCGATCCTGTGTAACTAGGATAGATGTCCCTTTAATCA
ACATACTGGAGTTTGGATTACTCATTGTATCGAGTTCACCATGATCCACTGTTAATCGTT
TAATAGCTGTTTGTAGTTTAAAGGAACGTTTTAAACCTTCATTTAACTCTTGCACAGTCC
ATTCACGATCACGCCTAGACTGGAACATATAACCAAACATACTTACAGCATAGTTAAACT
CCGACATCAAGTAATGTAGCACTGTTAACCTTTTACCATACATACTAGTTTCATCAATAT
CGGTAGCATAAAGATGATGAGCCAAACTAGTCATGATCTCGTAAAGAAGTTCCCAAATAG
TACTTACATTGACACCAACTGTTTTAAGTTCTTCAATAGTCATTTCGTCTAGTGAATTAC
AGAAACTGTGTAGGTGTGAATCAATGTTTTCCTCTACTTTACCTTGGTGCTCAAAATCAC
CAAACACCATGTGACCTAGGATAACACGCCATAAGTTGGTACTATCTACATACTCAGGTC
GAGTAAACCGCATTGGAAACGCATCGACCACATACCAGAAACCAGCGATAAGTCGTGTGG
CAAAAATAGATTCTTGATGACGAGGCATAACAAGAACCATTTCACCGGTGGGATGTTTGC
CTTTTAACGTTGCACTGGAATAAATATTCCACTTTTCACGTGGATATTCTTCTTCAGGAA
AATCAGACAGTAGCCCACACTTAATATCTACATTAGCCCATTGTTTGAAAGTTTGAGTCA
CTCCAAACTGACAGAAGAAATAATGAGCTAAACATGACTCAATGTGTGGTCGATTATCCA
GATCTCGTTTAGTACGCTTAGCCATTTCATTGTGAATCTGTGACCAGATCACATACATGA
TTTTACGCTGACCATTGCACATGTAATGGTGGTCAGTTTGTTTAAAGGTTAGCTTAGTAC
GACGAAACGGGATGAAGATACTATCATTAAGTACAGAGAACCCAACATCAGTTAATACTG
GTGAACCAATATATTTCGCACCATTGATAGTTACCATATTGCCTCTCTTAAAAGCTGGTA
ACAGCATTGGGCGAGGATAAAGCATTTCCCCTTTATAACTAAACATTAGGTCAATCATGT
ACATATCAGTTGGAGCAATATTGGCAATACGCTTAGCATTATATTCCCGAGTAATCTCTT
CAAAATGCTTAAGCGGCGTACATGCACGATTGCCTTCATATTTGAAGCCATCGGGAAATA
GAACTGAACTGATATCAAATAAAGAATCAATATAACGCCTAGTCATATTAACACCAGTAT
TTGGATCACGAGTCATGGTGTGTTCGACTGTGATACCATTTGCTAGTGTTGGATTCATGC
AAGGCGTATTTTCACGAATCTTTTCAATTAGAAATGGATCCGGCATTATAAACTCCTAAA
CTTTTGGCAAAATTAAATCCATCAGTGAACCTACAATTTTAATGGATGATACTAGATCAG
AGGCAATGGAGTTTAGATCACCCCATTTTAGACCATGAATCAATACAACATCTTTTGGTT
TGAATTCTTTATCAAACGTACCAGCTGCTTTTTCCTGGGCTTCCCTTTCCTTAATATCAG
CTGCTGTTAAGAGCGCAGTCTTAAGACGTTCAATAACTTTCTTATCTTTAATTCCAACCA
TTGCAGCAGCTTCTTCATGTGTTCTAAAGAAACCATTTTCATGGAACTGTTCAGGAGGTA
TTACATATCTCTTTTCGCCATTATGACTGATACACCCATGCTTACCACGGATATTAACAA
TAAGACCAGGATCTAATGATGGATTATAGATTGACTCAATGGCCATAATTCTGCCAGCCA
TTACATAAAACAATTTATCTGGTTTATTGATGTCAGTATTATACGTTAAAGAATATGTTT
CATTCCATACTTGATTAGAAAGTGCTTCTTCTTCTGGACTAAGTCCGCTGTAACCAGGAG
GACTAATATTTCTATTTGGATAAAATTCAAAATACTGACTATACTCAGTTTTAAATACTT
GGATACCTAAACTCTGAATCCAACCGCCATGATACTGATCTACTAGTTTTGATAAGTCAA
TTGAATACCAAATAGTAATCTCAGATAGCTGATTATTAAACTGATCGAATGTCTGTCGAC
TAAATGACTGAATATATTCTAACATTTCCTCAGGTGTAAAACCTGGAATAGATTTAGCCA
TAACTTCATAGTTAAAACTATCATCACCACTCCATCGGTAAATGACCTTAACTACTAGTC
GATTAGACATTACATACTTACGGGATGTCCCAATATTGATAGCGTGACCAGATGAATCGA
CTACAGTAATACGATGAAATGATTCATTCCGATACTCAACAAAACGTGAGACAGGGAGAT
TCCAATTAGCGTATGGATCACCCTTAGTGTATGCAGTTCCCGATTTCTTATCATACGGGT
GATCGATAGTGGGCTTAACTGATACTTGAGTAAGCCCCGCAATTTTTAATGTTACCATTG
AAATCTACCTTAATCACAACGGCCTCCAAATCTACAATGTTGGGGAGCCTCTTTTTAGTT
TGATGTACGGACCTTCCCTTGAAGGATTCACTCTTTTCCTTATTACTTGATTTCTGCCAT
TTATGCGGGTCACTCGAACAATATAACTTTTCGGTGGCCCGCTTAAACTGTTTTTCTTGA
GGGATCTCCTCAATGTTTTTCTTGGCATAATCTGTAATAAAGTCCTCTAGCCCTTTGGGC
ATAAAGGCATCCTGAACAGGATGGAATTCTTCAAAGAAAGTATTAATGTATTTATCATCA
TCTACGGTAGACATCTTATTATCTACCATGGTATTTTTTTCTATAAGAGATACCCTTAGG
GCATCTTGCCATCTTTCAATGACTTCTGGAAATAATTGAACGTTCATTTGGATTAACCTA
TTTTTAAATTAGAAGATCATGATCATTACTGTAATATAGATCTTAAACTTTTTTTAATGT
AATACAACATAAAGCCCTCCCCGAAGGGAGGGCCTATGTCTGTTACTCTTCTAACTTCCG
GGCATTACCGTCTTCCAAATAACGGATAGTCCCATTTTCTAGATAACGGTAACCCTTTCC
AGCTCACGGCTTAAGATCAGCAAGCTTACCGTTGAACTGAGTTTCGCCCATGACTTTCTG
GAAGCCAGTGATCTTGAACTCTAGTGCGAACGGAATGTTGTTGACGTGTAGGTTGAACGG
AATGGCCGCGATTTCGCGAGTAACCTGGTGACCACCACGAGTGATCGGTAGGTCAGCGAT
AACAGTAGATACGTAGAAGAACTGACCCCAGGAGAGGATGTCGTTTTCTTGTTGTACTGC
GCGGGTCGGAATTACAACTAGCTTACCATCGAATAGCTGGTTGTTGGTGGAAACGATATC
GTACTTGAGATAAGCACCTAGAGTACGGTCGTCACCTTTAGTCATTAGATAGTTAGCAAT
CTCTTTGTCGGAGCAGAAGAGATACATCGGAGTTTCATCCTGGTTACCGGAGATAACACG
GAAGGCAGCTTCAATGTTGGAATCACGGTGAGCCGGGAATAGCTCAGCTTTAACACAGTT
TAGGATCGCAGCGCAAACGTCGTCCCAACGATCTTTAGACTTGATGGTGTCGATGACTTT
CTCAAGATCTAGTTCTTTGTAACGATAGGTCGGACGCATTACAGCGGATAGTGCGCCTTC
GATGATACCAAACTTCGGACGGTTGTAACCATTGTGTACAACTTCACGTAGTTGAGCAAG
GTAGTTAAGCATGCGCTTAACAGCGTTGTTGCTGTTACGGATGTTGGTGTTAACAGTTAG
AGCTTTAACAACCTCACCCGGACCTTCATCGGTCATGGTGTCCATCGGTAGAGTTACAGG
AGCGTGCATCGGGATCGGATGACGGAACTGTAGTGCACGGGTCTGTAGTAGGTGACCACG
CTGACGACGGTTAGTGTTGGTGAAGCGAGTGTCTAGTTCGAAACCGATAACAGCTAGGTC
AGTTAGCTGATCTAGAATAGCTTTAACAGCCGGGTCAGCATTATCCATTACCTGACCATC
TTCGTTTAGAACTTTATCGACGTAAGTGTCGGTAGCACCAAACTTAGAATCGCCTTTGGA
TAGGCTGATAGTACCACCAAAACCAACGCTTAGACGTAGGCTTAGTTTAGCGGTCTTAAG
ATCATTGATAACACCGTCAGCTGAACCATCGATGAAAGTGGTATCGCCACTAACAACTAG
ATCATCGCTATCGAAACGGATAACAGCGTTCCGGGTATCACCAACTAGATCGGGCTGGAA
CACAGCGCGAGGTAGACGGTCAACCTTGAACTTAACAACTTTACCGCCGAGTAGAACGAA
TAGGTTCTTTAGACGACCAGCCGGATCAATGGTGTCAGAAACTTCAAGCATGCCACGCTG
GATGAGTAGGTTAGCATTGGAGTTACCCATTAGATCAAGACGTACGTTCGCTTTTAGCGG
AGCAGTAGTGATAGTTAGGTTCTGCTCGTTCTTGATGGTGTACGGAGGAACAAGAGCCGG
GTCTACGAAGAAGTCAGCATTGGAACCAGCCGGATCTAGGGCAGGAATTAGCGCAATGCT
TTCATCGTCGAGGATGGAAGGATCACGATAGGCTTCTACCATGTTCACTTCTTCATTATC
CATCTTAACGCCGGATACTTCGTGGTAAACGTCTTTCATTACAGCGATGTAAGGAAGAAC
CTGAACAACACCACCTTCAATCGGGTTGATAACGGTGGTCGGATAAATACGCTCAGCGAA
TTCATCTTGACGGCTAGCAGCTAGGTTATAACCAATAGTTACAACACGGAAATCACGCTG
TGACTTCTCGTTATAGTTCTCTAGGCCAACACCATTTTCGAATACTTGAATGCCACCAGC
AGGGCCATTGAACTGATGCTGTACAACCTTAACATGATCTTCATTAGAAGCTTTAGCTTT
GGTAATACCACGTAGTGCACGGATATATTTAGTTTCATCAGCACAGGCAATAGCAGCCAT
GGTAGCAGCGGTGATCTGGTTCTCACGGACACGTTCTTCACGAGCACGTACTTGAGGATC
ACTGGAACTTACTCGTTCGAAACCATAATCGTTAACGAGCATTTGCATTTTGTTATAAAT
TGCAGCAGCATCTTGAACGTCGGTAGCTTGTACGGTACCGAAGTTTTCATGTGAAATGGT
GCTAACAAGGGAACGACCTTGACTAACAACGCTGTCATTTAGACCAATTTCACTTTCTAG
GCGGCCAATGAAATCTTCCATTGAGAAGACTTCGTATTTCTTTTCGCCTTTGTGTTTAAA
CAGTTCTCTTAGGGCATGTACGCTCATTTTTTATTCCTACGTAGGATGAGTTTTTGGTTT
AAAAATTTGGGTCTACTAAGCATCTTTTCCGGAATCAACCTAGCAGCTCTCCAAATAATA
GGTAAAAATTTGTGTTGACGCCACGGATTCACGTGGGCGGGTGTTCAACATTTCATTTAT
CATACTATCAAAGAAAGTATGGTTAAGTTGAATTGAGACACGTCTAGGATCGCTACTTGC
AATGTGCGCACTCTGCTGCACCGCAACCCACAGATTCTCATCTACAGGATATAGGTAATA
ATCGTACACTAAATTACGGTTACCTGCATCCCTAAGTTCATTAGTCGCTAGGCGGCTCTC
TAAACGATTGTGTAAATCCTTCATCTTAAATTGTTCAGGACCGTCTATTTCACCTAACTC
AGCAACTTTGATTATCGGGCTTACTGCTAACTTAATATCTAGTTTTTCTGGTAATAGAAA
GGGGAAAGCAGTTAAAGCCTGATGTATCTCCGCAAGATCATTTACGGAGGTTTCGGAAAC
TATATAGTTGGTGTCTAACAATTTGTTAAATTGTACACCTGCTGTTTTATATGATTCAAA
AACAGCTTTTGGCATAACCAGAACTTGGGTCATTGTGAAAGTACCTATATTGTAACTTTA
GGCTTTTGGGAACTGTCTACATAGCAATATAGCAAATACATTCACTAAAATTACTAGCTA
TCTAAATGATACACTGTACATCTCTTAATTTCACAATTGGAAATCTATAAAAATGTCAAG
TCCAAAACAATTACTCGTTTCATGTATCACTCTATTATGTTTAGAAAATCGAGAAGATTC
TCCAGTGGAACCATCCAATGAATTGGTTACTGAGATTCTTGATACTCTAAATGATAAGGG
TGGTACATTAGATGTAGATCATGGTCGACAAACATTTTTAGATCTTCGTAATCTACTAGT
GGATCTAAATAAGAAACAGAAAGCATATTTCCCAGCTACCCAACAGGTATTACAATCACT
ACAAGTATGTTGTCGTGAAGAAAGTTATCTATACGATGCTGTTAAAAATGCAGTAGAAGA
AGAATTTCCAAATGGCATGGCATTAGCCATGCGTGTAACTTCATATCGTCGTGATCTAAA
TAGCTACTTAGCTGATGAAAAGGTAAAAGCTATTGTAAAAGAATGTTCATCAAAAATTCT
TTTCAATAGAGGCTCTGCTGATATTCCGGCAATCATTAATGAAATGGCAACCAGAGCTGA
CCCATATATTCGCGCGCGTGCTGAAGAGAAACACCCAGCTGAAATGGGTGCACTTGATTT
TGGTAATTTAGATTCTATTGAAGGCTTCTTTGAGGAAGCTAAAACTATTCTTTCCCCAAA
TGGTGCTTTCCGTACTGGATGGAAAGGTTTTAACAGGATGCTTGGTGATCTGGGTGCATT
AAAACGTGGTGAATTTATTATCGGTGGTGGCCTACAACATCAATATAAATCTGGCGTTTC
TATGAGTCTATTTTGCCATGTGTGTCTATTTAATAAACCATACATGCGTGATATCAATAA
GAAACCATTAGTAATGTTTATTACTCTTGAGAATGAGATTCCGGATAACCTTATTTTCAT
TTATGAGTATATTTACGAAAATGAAACAGGTATCAAGGTAGATCGTAGTTCTATTAGTAA
AGCTGAAGCAGCTGAATATGTATCAGCTCGTCTCCGTGAAAATGGTTTCGAACCAGTAAT
GTATCGTTTTGACCCAACTGAATTTACGATTGCTGGTTTAGTTAACTACCTAGATACATA
TCAAGCACGTGGTTATGAACTACAATATCTCTGTGTAGATTATCTAAACATGTTGCCTAA
AACTGGATTAGTGACCAGTGTAGCAGGTGATGACGTTCGTCTCCTATTTAGGCGAATGCG
TAACTATACTGCACCACGTGGTATTACATTCTTTAGTCCGCATCAGTTAAGTTCTCAGGC
ATTAGAACTAATCAGAGATAATATCCCACCAGAAGACTTTGTTAAACAAGTTGCTAACAA
AGGTTACTATGACGGTTGTCGCCGACTTGGACAAGAACCTGATCTGGAATTCTTCTTCCA
CATTGTTAAAGTAAGTGGTAAGTATTATCTAACCGTACAACGTGGTAAACACCGTAACGT
TGTAAGTGATAGTAAGCATCACTATTTTGTTATGCCGTTTGATCCAGACCGTATTGGTGG
TATTAGATGGGATATCGATAAAGAAGAAAACAATTACATGGACTTTGTACCAAGTCTTAG
CAATATGCAAGCCACTGGTGGAGATGACTGGGGATATTAACAACATAAAGCTCTCCCTTC
GGGGAGAGCACTATAATTTTAATAGGTAATCTAAATGAGTGCAATTGATAATTTAGTAAA
TACTTTAAGAAAGAATATTAGTGAATGTAGAAAGTTTAATCGACCCCTATTTGCACGTGT
ATATGCCGCATCACAAAATTACGATAAAATCCGTTATGCTGTAGCAGAAGAAGCCCATTA
TTTTAAATATGGTAAATATGCAGAAGGTAATAACCTAAATATCGATGAACTTATCAGTGA
CCTATATCTGTACTACACTATCTATGTCGATGTGATCCGTAAACCATTACCCGATCCTAA
TGGGAATTTTATCAGCATCGTAAACCAGGATCACTATACACACATCTATTGGGATTTAGA
TCGGTGTGAATCTATTCCAGATGATGTAAAGGAATTAATGTAATATATTTAATTCTGATT
AATCCATTCGTCTAATAATCAATAAAGCATTTTAGCCCTCTTAGGAGGGCTTTTATGTCG
TATGGCTAATATGCTAGTGGATAACGAATAAAATCGATTATAGAGCAAATTAGAAGGTAT
TAAAGGCATAAAGAAAGTCTATCTCTAGACTTATTATTTTTAATTAAAAAAAATATAATC
TATACTGTGTCTCCTTTTAGGGAGACAGTATGTATTAGCTAACGCTATTAGAATAATGCT
AATATTGTTTATTATTAGAATAAGGATATACCTTCGGTATAGTTTATTATGAAATAAAAG
CCCCTACGGGGCGATCCGATTCCTCAGTTCCACATTAGGTAAGATTCATGAGTAATATTT
GATTACCAGTATTACTCAATGATACTGGTAATAATATACTAGATTATTAACAATATGTAT
TGATCATTCTGATTAAGTCAAATTAGTTAATAAACAAATCAATTATCAACGAGTGATAAC
GGGTATTGATTACTAGCTAAAGAGATTAATTAGGATATCTACATGAGGAGTCGCTACCCC
ATGTATTCTGGAGTTTCTCTTTAGCGGGAGACTTAACTCCACTTGGGAGCCGCTTTAGCG
GGCGGCTCTCACTTAAACAAAAAAAAGAATTCAGATAAGTCCCTCTCCTTTAGGAGAGGG
GTTGTTTATCATAGGCTTCTCCTTTCGGTTACATAGAACTTCTTCTGATCCATGTCAGTG
TATGTTACTATGAAATTGTCGAAGGGGTTGATGGATACGGCTAAACCTGCGTTGTTGACG
TATCCTATTGTAGGGCCAGCCCAATAACCAATGAAGTTACCTCTACGATCTCGACATTGG
ATAACATTATCGGCTTGCGTTTTCGCAACCGACATGCTATGGGTTGATCCATCGGGTCTC
TTGAAAAAGAAACCTACTTCCTGCATTCTGTGCAGGTCAGGGCTAACATTGTTAGCGGTA
ACTGGAATTAACATGATTGTTCCTCTTGTTAATCCAAGCCCACTTGGGCGAAAATGTTGA
GTATTTTTTACTCAACGGCATATAGCCCTACTGCTGTAACAGTAGGGCTAATTCTTTTAT
TCTTAAAAGTTTAACTGAATAAATGCCCTCTCCCGAAGGAGAGGGCTTATGTCGTTAATT
TATTTAACCAGCGATGTTTCCCTAACACGAGAGATGCATAAATAATTACTGTAATCGCTG
GGGTCGTACTCATCATACCCTAGACAACTTGTATGAATATATTCAGTACTGATATTAATC
ATATCAAGAGTAATACCATCACCCCATTTCTTAGTTGCCATCTCGATAATATCGGCGATA
GTATTGGCTACATAACAATCACCCAAGATATAAAAGGTATCTTCGGTATCATCCGGACGA
AAATGACGAATACCATTCTGTGTATATTCACCCATGTTAATTATCCTCTTTTCCTTCTTG
AGTATGATGAGTGGGCTCTACCCAGTAAAAATCAATAAACAGATCAACTGGTTTACAGCC
AACCATCTCGCTGTCATCGGCCCATTTACTTTCATCTACGATAATTTCTTCATTAGTCAT
TTGTATTTCTCCTTATTTACTATAGTAAATATTGTTTAGTTACATAGTCCAATTAGTGGC
TTCATCAACCATATCAGCTGCTTCTTTATCAGAAGCGTTCCAGAGTTTATCAGCCCATTC
TTTATCCAGTTCATTTTGGATGTTTTCAAGACGCATGTAATTATGCCATGCCTGACCCAG
CCGAAGATTCTTGTTATTTTTGTAATAAATACCAAACTCACCGCGAACTGATGCTGGAAT
TGAAATAGTCATTTCATAGTCTCCTTAGTAACTATTTGACGTGCTTCTTTATAAGAAATA
TTCGGATTATCGTTGATGTATTTAAGAACTTTCTGATGTATAATGTTCTCTCGCTCAGTT
AGTTCTTTTACTACTCTTTTAAAATAACCTGGTGATCGTTTCATAGTCATGGTTCTTCGA
TTAACAGGTTATTTGCCTCGAAAAAGAATGGGGCTGACCAGCTATAATCTGGTGCATCTA
CCAGCCTGACACTGTTCTTTATTTTGATAATCTTACGATCATCCCGCCAGTTTTGTGGTC
CATGTTTAGGAACAGTGTAGTATTCACCATCGGGTAGATTAGCGGCTTTCTCCCATCCGA
GTTCTTTAACGCTAAACTTTTTATCAATAGTCATTTTCTTTATTTCCTCGCTGTCATGAA
ACCACTCGGTGACCGTATCAGGCTGCGTCCTCATGATTTATGCTGCACGTGGGATTACTG
ATATCTTAAGAGTCAGATCAGCTGCGTCCAGTAGGTTTACCAGGGCATCCAGAGAGAACT
TGTCAATCTTGCCTTTGAGTAGATCATTCAATCTTGGCTGCGTAACATGTAGGCGAGCAG
CAGCATCTTTTTGTGGAAGATCCCAGCCCTTGATAGTACGGGTGAGAGCCCGCATGAGTT
GGGAACGGATACGCAGGTTTTCGGTTTCTTCTGTCGAGTCAACTAGTTTATCCCAAACAC
CGGTAGTACGTTCGTTTGTCATCTGTTTGTTACCTCTTTAGGTAAATGTTCGGATAATAA
ACTACATTATATTTAAAGAAATCACTTACCTTGAATTTTATGTTCGGTGCAGTGATTAGA
TAACAATAATCACCGTCAACCATAACGCGTCGATCATGGCTATACACCCTAGGGCCATGT
AACGGAGAAGTTACATAAATCCCATTGGGAAGATTCTGTAAATCTTCTTTTTTCAGAAAC
TTACCATTAGGATTTATAGTTGATGGATAACACCCCTCATTAAGTTTATACATAAACTAT
CTCTCTTTATAAAACCACCTATTGTAAGGTGGTTTTATATCGTGTATAATGTTCTTTAAT
CTTACTGACCGATATAGGTGCTGGTATACAGGTCAATGGTTGGTTTGTGAATAGTCAGAT
TGTATTTTTTAGCTAGCTCATCAATATCCTTGGAGCAGTCAATAAACTTACCAGTCTCAC
GATATCGCATTACCCATGAACCCCGAAATGGTGTATCATCTAGTGGATTATAACCATATT
CACGACATACCACAATTGCTTCATTTCGCGGGGTGGCTCCAGTAGTGACAAAGTAATTTT
CATAAACTGGAATCATCGATACATCTTTATTGCTCATAATAACCTCTCTAAAATTACCTG
AAGGGATATGCATGATATTTATTTTTGTTTTTGACTGATCATGGAGGCATTGGTGAAGTA
ATATAACGCATGTTTCCAATCTTGAGGGTCATATGTGCACTTATGAGCAACCACATAATC
ACCATCAATTTTTTTGAACAAGTTTACCATGATCACCTCATCCATATCAGTCCCGTTATG
CACAGTTTGATAATTCAGTCTCACCAACATGTCATCTACATTGGTTTTCAACTGGAGACC
ATCTTTGAAGAAATCACGAACACCATCCATGATTTGTTCATTGCGAGACAGGATGTTTTC
AGAGATCTGTTCGAACATACCCCTTGCACCGATCAAACGTAATTGATCGTAATTGGGTTC
ACCCATGCAGATGGCCTCATCATCACGATTACACATCTTCTCCAGCAGAACGATAGGGTT
ATAATCCCAAGCAACCCCACCAGTTACAACAGAGGTCAAATAGCCATAGAGACTGAGTTC
ACTGTCACGCTCATTTGCAGGAATCATGCGGTTCATAATTAACGATCCTCGTATTGGTGT
TGCAGCATAGCTCAGCAAGTCTCTTGAGCTTTCTCAAAACTAGCCATCGAGCGGGTGCGC
GCACGCTGTAGAATAGACAAGCCGTATTTTTTGAACTTCTTCTTGTGGATGAAACCAAGG
CGTTCGTAGATGAAGTCTTTACGGTCGGCGCGAGGTTTACCATCAGCTACGATACCACGG
TCGAAAAGAGCCCAGAACAGAGCGCTTTCATCATGCAGGTAGTTGGCCATTTCCTGAGGC
CACTGAACTGCGCATAGAGCAACGATTTGTTCAAGTAAACCACTATCTGAGATGGCATAG
TACTCACGACCCTGAACATAGTTCATTTTGTCGAAGTTGCACATAGCCAGTCTGCCAGTA
CCCTGGTTGATTTTCTTTTGAATGTGTTCGTTCATGGTTTGACCTATTTTCAGATTTGGA
TTAATTATAGCTAGACGAAGGTATTGCGTTCGAATTGGATGGAATTACGTTCAACAATAT
CGTTGAGGTATTTATCATTGTCGATAAATTCCCCAGTGAGCATGTTACGCAGAACCCAGT
TCCCCCGAAGTGGTATACCGCTGTTAGGGTTGATCCCGTATTCACGCATCAAAATCCGAC
GATTGTGGTCAACACCTGGCTCCATGTAAGATTCACTGATAAAACATTCCACCATGTTGA
TAACTTCTTTAGATTCGCTCATTGACTTATTCCTTCTTTTGCAGGTATTATGTAATACGA
TAATGGATTATGGCTTTAACCATCAGATCCTTTTCACGCAGTTGGTTTGAATGGATTTGC
ACTACGACTACGTTTTTGATATGAAATTAGTGCACCCCATACATTATCGGCTGATACATA
TTCAGGCATAGCTTTTTCATCATTGTAACTGACACGGTATGCCTTAGCCACACCTGTGCT
ATCTACAATGTGAACTTTTTCAATGACAAATGCTTTTCCAGAAGGCCCAGGGATATGTAA
TCCTCTTTTAAAAAGTTCTTCAAGAATGCGTTCTAATGCAATTTTAGCATTAGGGTAATC
ATTATCAAGTGACTTAATACCACCTTTAATTTTATTATCTAGGTCACTGAGTGCCTTTTT
AAAAACACTAACATCATGATCAAAACATTCGAGAGTGTGATCGAAGTTTTGCATGAGCGC
TATTAGTAGACCATTAGGAAATACTACTTTTGACATATATCACCTATTAGTAAACAGTTG
GGAACGTTTGTAGGAACCAATCATTAATACGTGTATTAACACTAGCCCCGTGTTCAGGCG
CTACTTTAAAACCAGCGACGATGTCCTGTAACTCTTCAGTGAGTTTGTTGTGTACTTTAC
CTTCACAATCGATAAAGTCTTCTGCGGTTGGCATAACCTCAGCGCGAATAACAGAGGCAC
GCTTAGAAAGCATTGTAAGAATTTCATTAGACACTGAAGCAGCATTACGCAACTTATTGA
TTTTTACATCAATAACAGTTTTATGATAGATTTTACCAACTGCACACATTGTAACTGAAC
CAACAAAAATACCAAGACAGAGTGATACTTTATCAATATTCATTTTGTATACCTATTTAC
GTTTATTGAAGTGATTAAAAAATAATACGATGTAGAAAATAAAAGGGAGTCCGAAGACTC
CCATTATCAGTTACACATCTGACACATCATCGAACAGATCAAGTTGTTTCCGATTACGTC
GTTCTTCTTCTTCGATACGACGATTCTCTTCTTCAATCTCTTTCTGCCGTTTGACAAGCC
GCCTGTGCAGACTCATTTGCCATGCAGTATCATAACGGCATCGCACTGACTCAATATATT
TAAGCCCTACTTCATTAATGCTCTCAAGTAGATCCAGTGACACAAGTGTCTCCTTGGAAG
CATTAATACTAGCTTTAAGATAATGAATCATTGCAGTTGCAGGGTGAAAAGGTAGCTTAT
CTACATCGATCGTTTTCTGATATGCTTTCAATCTATGTATGATAATTCGTCCATAATAGA
ATTCATAGAGTTTTTCTCTAGAATCCCAAGCAAGCTTACCGAGTACGGCTCCACTTACCA
TCCCTAATGTAAGAGATAGTTTAGAGTTCATTGTTAATTGCATCCTTAATCCAGTTACGG
ATTTTTGATTTAATTCGATGTTTATCACTGACGGTATTAAACATATCCAATCCATCATTA
ATGACGTACATAAGCTCTACTTCAATAAAGTCCGTCATTACTTCCACGGTGGTATTATCT
GAATTAGATTTGATATAATCGGAATAGTCCTTTACTTTTTCACGGATATTCTTTTCTAAT
TTAATCTCATTATAAACAGTCACACCGACTTCGAACACATAGATGGTACCAGCCAGCATG
AGACCAGTTTTTACAACACTACCGATAATGGAAAGCATAATAATATCCTATTAGTTAATG
AATGTAGATTTATTTTGATTATATTAAAACTTGATATTTAGAACACGTTTAGCTATTACA
ATACCAACATATACTCCTAGTGCTATCTCCGCATATTTGGTGATGACGTTTTTAATCTTC
TTGTGTTTAGGTGCCTCAGGAACATCCGCGATAAGTTTTTGAAGTTTAACTTTATCAATA
TTCATTTGTATTTTCCTTTTTACGATTAATTGAAGGTTTAACAAACTCATTCAAATATAA
TTTTATTATTGTAATTATATTTGAATGGGGCCTCACTGAGGCCCTATCCATTTTTACATC
TTACGAACTGTATCGAATACTTGATTCGTATAGTCATCGCCATAAATGATCGACAGCTTA
TCACGAATTACATCCAGTCGATGTTTTTCAATATTGCTCTCAGCAACAGCACTGTCAGTT
GTCTCAGTCATTGCAGTATTGACGAACATCATGAACTCATCTGATTCGCCATACTCTTTT
GAAATACTTTTAAACTGATCCCTGAAACCCATGAGCTTGTCAAAGTTGGCTTTAGTTTCC
TTAGCCTTCTTGTAGATAACATACCCAGTTCCAGCTAGTGATGCAGTGGCCAGAAGAAAA
GATACACCTTTTGCAATCGTATTGAATTTCATAATAAAAGCCCCTTTTTACATTAAGTGG
ATGATAATGTTTACAGTATTGTCACAAATGTAATATACTGTTTAAATATGTTTGAATATA
CGACATAAAGCCTTCCCCTGAGGGAAGGCCAATGTTAAACCGATGAATCCATCCACGGTG
GATAAACAACCACAATCTGGTTAGGCATGTGATTAGACCAGTCTTCAATGATAGTTCTAA
CATCAGAGAAATCTAATCCACCATTAGCACACCCTAGAGGTGGTATGATAATTTTCCAAT
CTGGATGAATACAGTCTTCTTCATTTTCCGCAGAATCCGATACGTTCTCCATCATCCACT
CTAGCCCACGTTCAATGAAACTAATTTGAGATGGGTTCTGCCAGTTATCCTTAGTAGGGA
ACATCATGAAACGTTTACCATCGGATGCTTTGTAAATAACCGGATTACCAATAGTAATAA
TTCTCTTTTTGCAATCGTGTTTGTATCTTTCAAAAAGTTCAGGATACTTTTCTTTAAACG
ATTTGGCTAAACCAGCGCCCATGACGCCAACAACATTAACAGTGATTAGATAAAGATCAC
CAGGTACATTGAATATATCTTTACCTATAATAAATTCAGCCATGATAATGGCTCCTTCTA
CAAGAGTACTAATGAGAAGATCAAAACATAGACCTTCTCATTAGATTGGGTGCATTTCCA
GGCTTATTGGATTTCTGGATTAGCGTTGTAGAAGCCAATATAAGCCTTATTAAATTTCAA
GTAATAATTATACTCTGAAGGCTCACCAGTTATTTCCCTAAATACCTTAGCAGTTATAAC
TTCGAACTTAGAACGCCGTAGCATTGGGAATAATTTCTTACTGCGCGATAGCTCACATAA
TCTAATCGAGATAGCTTGATCTTTAAATAAACCTGATTTGGTAAAAAAAGCGCCTTCATG
AAGCCATTGAATTACTTCATTAAAGTCTGCGCGCCATTTCCTATGCTCATTTAAAGTTTT
ACCATTTATAAGATTCCATAAAGCTCTAACTTTAGATCCAATAGTAAAAATACCTATTAC
CATTACTGCTAGACATATAGCAATTATAAATGACATATGTCCTCCTAAATACCATTGAAG
ATTAAAGTTCCTATTAGTGAGAGAATGAATAATACGACCACATGATGAAGTTTTATCCAT
GTTTCAGGTACTACCGGTTGTTTAGGTTTATTTAAAGGGATGTTTTCCATGTATATGCTC
CACATTAGTTCATATGTGTAATATACTGTTTAAGATCTTTCAAATACACCTTTCCAATTA
GTAGGTCTTGGATAGGACCTAGGTAGTGATTCAGAATGTATAATGAATCCCTCGATATCC
CCTTCTTTTGTTTTAACAGCAACTATATCTTTGATAATACCTTTTATTTGTAATAAGCTA
TTCATAATCTCACGATAGAGTTGATCAAATCTATTTGCAATGATATTAGGATTTATTGGA
TAACCACAAATTGATTTATAGAAACTACACATTACATCATTGTATCTATCTGGAACACAT
GTCCAAAAGAAATTATGTCGTTCTTTAATATAAAGAATGATCAAGTAGTTAAGATCATCT
TCATCTTTTGCGGTATAATTAAAGTATTGTTTTAATTCAGAAAAGAATTGTTGTGACCAC
CTCACATCAACTCGTATAAATAGTTGTTCCATAATACACCTAAATAAACCCTCCACTTAA
GTGGAGGGCTCTATTTCATTTTGGTCAGATCTATTTAAAAATATAGCTAATGTAACAACA
CATACATATCGATGCGGATTAGGATTATTATCACCAGCTGCAAATTGTTCATCTACACCT
AATAATACACCGTAGTAGGTTTTAACAACATTGTTGTCAACTGTTTGAAACTTAACAGGC
ATACCTGGTGTTAAGATATCAGCATCGCCATGGTTCCATTGTATTTTAAGAAACTGGCCT
TGACGCTTTGCTAACTCTGTATAATGTTTAAATGGATTTGATGTGGACCTTTGTTTAGTC
CATTGGATATTTTGGAATCCAGTTTTTAATCCATCGCCTCTAACCTCATACAAGTTACTA
GCTGCATCTACAAGCATTTTATTATCTTTACTAATAGTAAAGTCATCTAATAGCTTATTT
GCATCTGCGAATCTATAACCACTTCCATTATCAACCATCTCATAAAGAGCTTTATCTAGC
GAACTAGCATCACCTGTTGCCAGAATGGTTGTATAAGTATCACCATTCACATACGTTTTC
TCAACTCCGTAAAGGCGATCATTTGGAACGTTAATTATATTTAATGTCTTAGCCCCTTTT
TGATATTTCTTAGTATCATAAAGAGAATAAACATATAAATAAGATCCCTGAATATATCTA
CCAAGACCAGTGGCATAAACACCACCTTCCCTGTTCTGAAGTATATTCGGTAAATCTTTT
AATGGTGTACCATCAGGAATAGGTATTACTGGACGAACTTCTGTATTCCACCCAGGTTCC
ATATTGATGTCAATTACTCGGTTATCGTCAGCTTCGGTCAACTGGGATATTGATTGTTTT
AAGAATGCTCTTAAAACATCTGCTGTTGTTACATTTGTGAAGTTTTTACCAATGGTTATC
ATCAGTGATTGATATACACTTTCTTCAACAAGCTGAAATCTATAAGGCTTATAACTTATT
TGGTTCATAGCCTCTTTACTAGTCAGTTGTGATTGTTTATTAGTTAGTGTAATATCGTCT
TCAGGTGATAATACAACGAAGCACCTATAGCGTTTTACAGAGGCTTTACGAGACGTATCT
AAAAGAGATGAGTTTGGTAATAATGGAATAAATTTAACATCGCACCTAAGTTCATCTCGA
CGAGGTAGTATATCGAATTGAAAATCACCAGTACCAATAAGTAACTCTGTTAAGATAAGG
TCACCATAAGAACCATCAGCATAGTTTCTAAATGTATCGAAATAATCTACTCGTAATGGC
ACTACCCAATCGAAATCACCTACTAGAATTTTAGCTTCGATTTTATAAGCACTGTAATTA
TTAGTAGAGTTAATAATAGTATCAACTTCACGCATTAATGAAGATGCATCAACTGCCATA
TCAATCCTCCATCAATACGTATTTTTCAATTTCATCAACTAGTGACTTATATGGTTTAAT
ATTTTGTTTTTGGTCTTGAACACGCTTGTCTAGGAATTTCCTAGTGGCCATGGAGTTACG
ACGCATGTTCATTGTAATGATTGAATCAAAAACACTACTACGTCTTTCAACTTGTGGCTC
TAATTTCATGGCTGTGAAATATACTTCCATTAAAAGATCTTCTAAACCACGCAAATCTTC
AATAGGTGGAAATGTATTTGGATCAGCACCCATTAATGTCAGATCATACCAATCTTGAAA
ATGTCGTTGGATATCATGATAAATCTGAGCACAATCGGAATGTTTATAAAAACCAATAGG
GTGTCCTTCAGCTAGTATTAAAGCTAAACCAGCGGCTGTCTGACGAACAGTTTCTAACTG
GTTAGTACGTAGCCTATCTACTCTTTTATCGCCAGTTGTGTTAAATGGATTCTTACGAAG
ATACTCAGCGCTATATCCAGCGATCATAGGAACCTGCACCATGTATTGCAGGTTCCATAT
GCGCCATGCAGCTGAGTTACGAGTAACCAGCTTATAATCCATGGTATTACCTCAATGAGT
ACTTAATAAGGGATAATAAGACGATATGGTAATAATATCGTTCTAGGTTATCCCAACTAC
GTGAGCATTTTGCAACTTCTCTTAACTGGTCAGCATTTACTGCCTCACCATTAATCATCG
CCCAAACACATAACTCTAATTTAGACATAAGTTTCTTATCACCACGATAGAAAGCTTCTG
TAAAAACATAGTAGTTATCGCGAACTATTGGATATATATCAGCAGGTATTTGCCATGGTT
TAACTCTATCCTCATATTGTGGATCGATTCGTTTAAACCATGGTAAGTTCCTTTCAGCCT
GGGTCCTATATGGACCTGGCGCTTCACGCATGGGTCTGCCTTCACGGAATGGTTGTCCAT
CTGGCCGACACATGGTTTCACCATCATAACGAGATTCAACATCAATAGGGGCATCTATTG
GGAATACCATCCTAGCTATACCTGTAAAACCAATAGCCTGGAATACTGGTTTCCAACGTG
AAATCTTAGTAGTAACTAAATGTGCTTTTTCTGTTGCATAACACATTCTAGCTACATCTT
GTCTAACAATAGCATCCCATACTGTAGGTTGTGACATCACTGGTTCAGAGAACACATTTA
GTAGCTTTATTTTGTACATACGTGGATCAGCTGAGGCATCTACCATTTGTAGGAAAGCCT
TAACTGCGAAATGGTCGTATGTCTTTGTAAGTTGGTCAGGTACTAAGAATGTTTGATGTT
GAGGCGAATAAAACTCAGTGATATAGTTCCTAATTATTTCCTTATATAACTTAGTCAGAT
TAGTATTATCTTCTTGTTCTTGCTCAGTAACAAATGGACCACATCCATTCATCAAAGAGG
TTGAAGAGTAATAATAACTCATCTGTGATTTAGTTATTAAATCATCATATTCCTGCTGAG
TTACATAATGGATCATCTTCCATGTTACAGCATAGGTTGAATCACGCAAAATGGTCTGTC
GTCTAACAGACGTGATGGTGAATAATCCTGATCGTCCATCACCAACATCCGCTATAAACA
TATCGTTTTTTTGCGGAACTAAGAATGGATATGTAAAGCCTGTACCATCCACTTTAAATT
TAGTGGTGTTTTCAGCTTGGTTATACTGAAGAGCTTGTGTTACTTTTAGATCCATTCCTT
TAATAGCAATATACTGCTGTTTATCTGGACGACGATGAATATCCAATGCAGTTGGTTCTT
GATCTTCGCGTAGTATTTGTGAAAAGTAATCTACACGCCAGTTTGAGCCAGTTATCCATG
CCATCAAATCTGAACGTGGTACATACTTGGTATCAACAGCAATACCACGGAAAACATCTG
GACATTCTTTAGGGTTAACTGGTTTAACTTCAGGATTTACTGGAAGTTCTTCATCTTCAA
TTGTTAATAATGCCATTATTCTTTTCTCTCAGTAATAATGTTTCCATATAGAACTGTCAA
TGGTCCCATTAAGTAAGTTGGATCCCAATCTGGTCCAATACGTGTATTCATTTCTTCTCT
GGCATCGACAATATCTTTCCAGTTAATTACACCTTCAGTTGTCGGATTAAGGCACTGTGT
ATGACAGGTAGTACCTTCCCCTGGACATTCCTCAGATGGTAACCTATCCCTACCTTTACC
TAATAGCTTAACTTCTTCAAATGGTTTGCGTCCAATACCGACACCAAATAAATTAAATAT
AGTCCAACATACACGTGGATAACGTTGTAAACAACGCCATCTATCATCAAATACTAACAA
TAGATTTTTATGTATTGATATCTGTACATGATAAACTTTACATGGATCTAGCTTCCATTT
CGTTTTAAGCATCAATCCATCTAAATAACATTCAGCTCTGGTATAGTTATCATTTTCATA
TAACCTAACTTGGAAGATACCATTTTGGTCTATTGCCTTTGTACCTACCCCTTCAAAATA
TTCTAAGAAGAATGGGCTAAATGAATATCTACCTAATTGGTCAAATGACATTAGTGTACC
GGATTCAATTTCATCAGGGCACAGTGTTAATAGACCACTAAAGAAAGTCAACTGATTGAT
TGGAATATTATCAGTATCCCATGTATCAATATCTGGATGCTGAATATAAGGTATCTTCTT
ATCTCTAATTAATAGGAACTTATGATAAAGAGATCCTCTAAACTGGCTAACTTTACGATC
TACTTGCTGATAGTTCTCATAGATGTATTTAGGTCTAATTACAGATGGAACTAAACACTG
ATGCACTGTTAAAGGATATTCAATCCACACATGTGTTGGTCTAGCATATGTTAGAAAATA
GCTAAATCTAACATTATAGGAACCATCGCTTTCACTGTCATATTCAGGAGTAGGTGGTGT
ACTGGTATAATCATACCAACCAATAACATCGACCTGTCTTTCGTATATAACCAGTGATTT
ATTTTTTTGACTAAGATCAGCTAATTCTGTAGTTGGGAACGTAAAATATTTATTCAAATA
TTCTGGGAATGTTTCTTCCATTGGCCAATGTGAATTTTGAGCAGTATTATAGATAGCTGC
TAATATTCCTTGAACGGGCATTGGTACATCAACATGATAGGTTAATGTGTGTACTAATTC
CTGACCATTGTTAGAAAACCATAAACGCTGTTCATCTAACCATCGTTGCGCTAATGTAAT
ATTTTTAGATTTATATTCAATGTCAAATCTAACGTCCACAAATCTACGAACGGGTGTGAT
ACGTACACCACGTGTTTCATCTACGAACCATGGGAAATTCTGATGATTCTTAACATCAGT
TGATAATGTATATCGTTCATCAGCTACTTCATCATAAGTGATAATAATGTGGTCTTCTGG
ATCGAATTTAACCACATTGGCACTATCACAGCAATTACCAAACATACCGCCATTCATTGG
CACTGTTTGAACATTACCTGGAAATTGGACAAGAGTGTCTTCGGGTAAACCCATCATCTT
GGCCAATTGCCCGATGATATCAGCTGCTACCCGACGGGTTACAGATTCGTATACATCAGG
TAGTACTAGTGTTGTATTAGGCATGGGTTTAACTCCATGTGTTTAAGATCATAAGATCAT
GGTCTAACAGCATAAAAGGGAGCCATGAAGGCTCCCCGTTATGTTTGTTACTTAATGGCA
CGATTCACAGCAGTGTTAATTGCATTGGTAAGAGCTACAATGTAATCAACTAGAACGGTG
TGGTTTTCAGCCGATTGGCCAACAACTTCCCATACTGCACTAGCTAGTTGCCGGACTTTC
TTTTTACCGGCTTTACTATCCATGCTAGCCACTTTAGAGATATCACGGTTAGCTTGGTCA
AGACGACGTACACCGCGGTTACCGCTAACTTTTAGAGAGGCATTACCAAGACCAATTAGA
ACTTTAGAAAGTTTCATTAGTTCAGTAACGTCCTTACGGCTAACTTTAGCAGTACCAGTA
CCAGTAGCACCCCATTCACCAGTTGGAAGTGCAATAGCAGCTAATCGTTCAATATCTTTA
CTAGTGCGACCAGTTACATCACGTCCAGACTTCTCGAAGGTAAAACTACCATCTAGTTTT
CCACTAATGAATGCGCTTTCAGGATATTTCTTAACAGGTACTTCTAATTTAGTTGCTTCT
TCTAGAGCCTTATCAATAGCTTCTTCACTATCAGCTTCAATAGCCGTTGCTAGATGACCA
ACTAAACGTTCGATGTATTCACCATCGATTTTAAAGCATTTGTTAATAATATCACTAATA
CGACGAACCTCTCCAGCAACACCACGTACTAGATTATCATCACCTTTTAGAATACCATTA
ATTTTGATTTCTTTATTTTCAAAATTATCGGTGTCCTTAAGCTTAACTAGAAGTTGGTCA
GCTTTAGTATTAGCTGCGGCGAGTTCTTTTTGGTTAGCTTTAACATGGATGGATGCATTT
GCACCTTCAGCAATATTGTCGATAATATTTCGAACAAACTTACCAATAGTGCTATTAAAT
GATTCTAGTGAAGTAACCATCGCCTTATAGTAATCTTCTAGATTACCATAACCATAATCT
TCCATTGCTGGAATACCAGGTGAATAGACATCAAAGGCATTGTGTAGACGTTCTAAGCAG
CTATTAGCATGTACAAGGAACTGAGGACTATATTGCTTATGCTCTAGTCCATAACGCAGA
ATCTCGATCATAGCTTCATTTGATGCCTGCTCTTCAAGTAATTGACCAATGGTCTTCTTA
CTGTCCTCAACTTCATCTTCAAGATCATCAGCTTCAGCAACTGCCTCATCAGCAGCTTCA
TCTGCTTCTTCAACATCATCTTCAGTTACTTCTACATCGACATCTGGAATATCAATTTCA
CCTTCAGCTAGTTTCTCTTCGATGTCATCTTCAGAGACATCATCATCAGCTTCAGCGACA
ATTGTTTCACTATCATCTTCAACTGCACCTTCTTCAATTGTTGGTTCAGCAATAGTAGAT
GCTGTTTCAGTTTCAGCAACTGTTTCTTCAGTTTCAGCTTCATTAATTAATACATCAGTA
GAATCACTATCATCATTAGTTAATTCTTCTTCAGCTTCATCCCGAACTTCTTCCAATTCG
CTTAAGCCGTCTTCAGCTACTTCTGCTGTTTCGGCAGCTTCTTCTACAATTGGATCAGTT
ACAGCTACTTCTTCATCAACTGCTACGATTTCATCCGGTTCAGCTTCAGCAGGGATACTT
TCAGATTCCGTCTCTACTACTTCAGCTACTTCACTATCAGTTGTATCCGGCGTGTCTTCC
ACGGTTTCAACAACTTCTGTTTCAGCATCAGCTACAGCTGGTTCAGTAACATCGACTTCT
TCAGTTTCAGTAATCTCTGCATCAGGTTCATCATTGGAACTTTCATCAGCAGTTGTCTGT
TCAGCACCGGCTTCTTCCGGTGTCACATCATCATCGATACCAGGAACTGCTTCGCTTACA
ACTTCACCAGCATCTAATACTTCATCCGGTAGTGTATCCATAGTGGATGACACGGGTTCA
TCACCAGCTGGAAGTTCTTCAGACAGTGGCGCAGGAGGCGGAGTTTCGTCACCAAGTGGG
GTTTCTTCAGTGGGAGTTAATTCCACTGCTGTTTCACCAGGTGCTTCTGCAACAGGTACT
AGTTCAGTATCATCAGCCGCATCTTGTGCAGCTTCAATAATTTCAGTGGTTGAATCTAAA
ACCTCTTTACCACCTTCAGAAATTGTACCGATTTCTTCAGCGATTTCAGCTTCAGCAACT
TTAACTGCTTCTTCGCCTTCTTCGACACTAGTGGTAGCTAGCGGTTCAGGAATACTTTCC
TCAACAACAGTTTCTTCTGGTACAACTTCTTCAACAATTACTGCTGACTTATCAGCAGCA
TGTGCTGTTTCTTCATGTTCTTCAGCAGAGATAATATCAGTATCTGTTTTAACAGTTTCA
TTCTCAATAGTTGCAGATGCATCATTATCTTTCTTTAAATCTTGAACTAGTTCATGATCA
TCTTTAACAGATGTCTCGACTTTGGATACATCCTGTTCCACAGCCGTACCATCTTGGTTC
TCAAGGCTGGCTACATATTTGTGAATATTTACCATCATGTTAATATACCTTCTGTACAGA
GTTTATGAGTTATGCTATTCGTCTTAGTCATAACTCCAAGCGACGAACGTTAACATTCAA
AATATTGCGCTGGAATCTTACTTTAGATTTTCATCGCATACATTGAGTATGGCAGTCAAA
TTACGACATGTTAATTTAAATAGGTCGTGATAAGGAATAGCCAACCAATCAACATATGTT
TCTACCAATGAAACAAGTTCTTGTATTTCTCTATCAGGTCGCCCTTCATTACGAGCACGT
GTTAGTTCATCTCTAAACCTGATTAATGCGCCTTTAAGAGTCCTTAACCGTTCATTTCGA
CGAGTACGCATTGACGCAGCATTTGCACGATTGATCTTATCAACTACCTTAACAACTCGA
TCAATAATACTTTCAGTAACTTGATGGTTGAAATGTTCAAATTCTATTTCGACTGGTAGT
GGTCTAGGGGTTTCTTCAGATGGAACTAACCGTGAAGAAATATCCCTACACTGATCATAA
GCACTTGCATTCTTATCGGGATTATAGTTAATTACAATTTGATGATTACCTAGCAAATGC
AATGATACAAATCGATTACCTTGATCGCGATAAACAGATGGTGTGGTTATTTTAGTTGGA
GCACTATTTAATAGTAGTTCTGCTAATGCATCTGGACTAGCATTTGGATTCATATACGAA
GGTATGATATTAGCTGGCCTTAGAATCACATTGTCTAATCCATTAATATATCCATCAACT
ACTGTCTCCAATACGCGTAAGTGAGATAATAAATTAGCACTATTGGTAATAGGTTTATAT
CTAACACTTAAGTTCTGAATACGAGTTGTAATTAGGAATGTAGGATTCTTATTTTTACGA
GTTCTAGCACGACTATTAACTCGTAAGTTTTCTGCTCGAATTGCAACATATTCAGCAGTA
GCTGCTAATACTACTCCACTATCCATCAGTTTCTCAATAGTGGTAACAAATAGTTTTATA
ACTCGATCAATAACATGTTTAATAGTATCACTAATATCTTCAGTAGATACAACATCCTCT
AATTCTTTGATAACCCCTTGGATCTGATTATAATCTTCCATGCCGCCTAATAGATCCATC
ACTCGCCTAGCTAACGCTTCATCGCCTGATAATAAATCTGCAAGTCTTTTAGGTTCATCA
AGATCAATAATCATTTCATCTGGGGTATCTTTATAAATATCAATTAATTCCATTGCTAAC
TCCTAACATAAGACGGGACTCTTCCGAGCCCCGTCGATATGTCGTCACTTATCATTATTC
TGCTTGGAATTTAGTGTTAATTACACTATTATAAAGTCCAGTTGCAGTATTTGCGTAACG
CATCGCTTCACGAATAATGGAGTCATGTTTAGGTGCACTATCAGTAATAATAGACGCTAC
CTTACGTGATACTTCTTCGATCTGTTCTTTAGAAACATCGTCGATTTTATCACTCTCTAG
TTTCCGTGAAAGTTGATCAACATCATCTTCAAGTTGCTTAATGGCTTTAACAATATTCCG
ACCGCCAGTGATAACACCAGCTGCATCATCAACCAGATTACGTAGAGCTACCGCTTCTTT
CTTAGCTTGACTCTTATCAAAGTTCTTTAAAGTCACTTCGGTGCCAGGTTCTGCGGCTGT
CTCATCATCCGTTAGATATACTTGAGTAGCAGTCGTAGTTACAAGCTTATTACCCGGTAA
AGTGTTAGATGTACTGGCGTAGTGTTTGAGAGGCCCATCTTGAAGTTTGTCCATCTTCTC
AACTAGTTCACTAGCATCATTACGCTCTGGATCCCAAGCTTTAAGCACAGTGCCAATATT
CGTTAGGTAGGTGGCATAGTCTTTTGCATAAACAGATGCCATCCATTTAACGATCGGACC
ATATCCAACATTAGCTGAAGGCTGAATGAATTTACCATTAGCAAATATGGGTCCACCATT
TTTAACAGGAACCGTTAATTCACTGGGTATATTTTCAAACTTAGCATTTTGGATAGCTAA
AATGAGTTCATCAACTTTTACACTCACACGCGGTACACTATTCTTAAGACTATTCCATAC
GTGACTAATAATTTCACGTAGTTTAGCAATAGCTTTCTTGATATAAGCAAGCGCCTTACG
GCTTAACTCTTTAATATCCTCAGTTGAAATCTTAACTGGATATAAATGACTTTTACCACC
AGTTGTATAGTTTTCTAATGAGCTAGTTAAAGTATTATCACCAAGTACATGACGATGTGG
TAAGAGTGCAGAATGAATAAATGCAGCTGTACTTTGACTGATACCATTAATACCAGCTTG
TCGAACAAGAGCTTTAATTGACTCTAGTGCATATGCGGTATTTAGACCTTGATTGATATC
TTGCACTGCTGTAGTAACTACTTGTGCATTACCAATGTCCATGAAATCAGTAGGTTTATC
CATATCCACTGCAACAGCAATCTGCGGTACCACGCCATTATGCGCTAGTGGAATAGAACT
ATCTTTGGCAGCCTGATCAGCGATTTGCTGAGTTACTGTTTCACCACCTTGTGGATCTTC
TCCTTCAGCACCTGCTGGAATAACTGTTTCAACAGCAACATTAGGTTGTTCAGGAATAGG
AGGTACTTCTGTTACTACATCTGTTTCAACTACGGTAACCGGTACCGCAGGTTGTTCAAC
TAGAGTAGCAGCTGGAGTCTCAACAATAACGCCGTTATTAGGAGTATTAACAATAGTGGT
ACCTGCATCCACTGGTGCAGTTGGTGTTACACCAACATTATTGTCAGCTACATCTTGAAT
AGTATTTTCAGGTACAGCATCTTCAGAAGTGCAATCGCCACTAGTCATGGCTGCTACTTT
ATCAACTGCTGTTTCAGGAATAGGTACCTCAGTATAGTCTTCTAGACTATCTAGATACTC
ATTGAGTTTAAATGTCATGTTATTTCCTCAAGGAATTTCTTTGCGCGCGTTTAACAATAA
ACCATAGAATTAGGTACTTAGATAAAAATGAACACTTAGATATATTTATTGGCCATATCT
TTCTAATTCCATATTACAAACAGCATTTCGTTGCTTTGCAATACGTAATAGGTAACTAAT
TAGTGTTGCGTATTCTGGTGACATATCGGTAATAAAGCCATTAACTAAAGATATAATCTC
ATTTGCATCATCACGAGCACCTTTCCAAATAGCTTTAGACCTACGTAGCTCCATGTCCTC
AATGCCCTGTGCAACCTTCTTCAGTTGTTCAAGGACATCTTTTTCAGCTTTAAATAGATC
ACCTAAACGACGAATAGCTTCTTTATTAACTTCTAATGTTTTACGCACTTCAACAGGCGG
CCTTGGCTCGATCGTGATTGTATCTGGACCATCACCATGTTTATCTTCACCAAGTCGTGG
AGCATATTTATCTTTCTGGAATATAAGTTTACTATTACCAGGTAGAGAAGTATCTTTTAC
TTTATCTAACTGATTAGCTTCACCAATAAATTCAAAAGCCTCTTTAAACTTATCTAGTTC
ATTATCTTCTAAGTCGTAGTCTTTAATTAATTTAACTACTTTATCAATGTTTGATCTAGC
TATATTTGGCCATTCTTTAGCTAGAAATTGACTAACTTTTGTTTCATTAGTTAAATCGTC
TAAAACAAATTCATCACCAATGAATAACATTTTAGGATTGTCAATTTTAATTGACATATC
AAAATCAGGTGTTACCCTTTCATTTGATACAGTTGGCTTATTGATTAAACTTTTCGCTTT
TTCAATTAACTCATCAGTTTTATCTTCAACTGATTTGATTCCAGTTAGTACACGCTGTGC
CGCTACTTTAGCTTGTTCAATAACATGGACTATTAAAGCAAGTATCTTTTTACCTATACT
TTCAATACGTTCTTTTATTTCTTCTTGAGAAATATTTACAAAACGCATAGCGCTGCTAGG
GGTTGCATTAAAATCTTCAATACTAATCTTTTCAAAGCCGATAAGCTTATCAATTGATTC
TAATGTAACGTGCATAAAAGCAGCTGATTGTTTAGTAATACCTCTATCACCAGCGGTATT
TAATAAATCTATAAAACATTCTAAACTAGTACGCGCATGCAGTAATTGATTTAGCTGATA
TGTCATGTTTTGAGATGGAGGTGTATCATACGCAAAGATATTTAAATGATCATCATCACT
ATCTAAATCTTTACCATCCACTGGTGTAGGTCTATTATCAGGACTTTCTCGGGTATCATG
GGGTGATGCTGGATATTCTTCTGATGCGACGTATGTTTTACCAAATGAATCAACTTCAGT
AACTAAACCACGATCTGATTTCCAATGGATATTAGGTTTATGTGCTTCTTCAACATCTAA
CCCATTATTAGTTGCTTTTACATCATTTGGTAATTCATCAATTAATTTGGCAGCTTCTTC
TGGTTCTTTAGATATTTGCTTGTCGTCTGTAGCAGCGTGCTTTAGTTTGGCTTTCGCGAC
TTTTTCTTTTACTTCGACAACACCATAACCTTCTAAACTATCAAGTAACTTATTTAAGTT
CATTTACAATTCCTCAAGAATACTTAATTACATAGCATAAGAGGAACCCCGAAGGGTTCC
TCATTATGTTTGTTACTTCTCATCATTCTCAGGTGCTTTAGAAGTTTCTTTGCCGTACGC
AGCTAATTCATGTTTAACAATAAATGTATACTGCGCAGCGATGCATTTAGCAATATAGTT
AACTAGTGCACTATTATCAATACCACCAATATTCAGTGCATCACGCGCTGTTTCTTTTGA
CATGCCTGCTTCGTCAGGATTCCAATCAGCTAGGGCCTTTTCAAGATTCTCAGCAATCTT
GTAATCAATCTTATTAAAGTCCTGTAGTTTAATAACAAGACCTTTAAGAATATTAAGATG
TGCTAAGATTTTACTCGGTGCACGGACATCAACTTCAACTTCATGTAGATCCGTTTCCCC
ATCACCCTCAACAACTTCCCACTTATTCACACCTGACTTAGAAGAAGTAACTTCTAACTT
AACATTACCAGGAGCTTTCTCAACGGCTTTATTAATATCTTCAGCCATCTTAGCTTGTAA
ATCACTGGTGACTTGATCAATATCAATAGACTTGTCACCTTTGAGATAACGTACTCCAAG
TTCGCCATATTTCCTAAGAGCAGATACATACTGAGTTGAGAATACAGTCATGAAGTCTGC
AATAGTCTTCGGATTATTTGCAATAAAGCCACTACTAGATACCATATACTCAGAAGGAGT
GAACTTAATCTCTTCTTTCTTTAGATCACCGGCTTTGAGTGATTTAATAGCATCAATAGT
TTTAGTAATACGCATATCGAGTGCAATCAGCTTAGGATTAAAATCTTTAATCTTCTGCTT
TATCATTGCAACAATTTTCTTGATAATCTCTAAAGCTTTCTTACCTGCTTCTTTTAGTTT
CTCTTTGAGATCCTCAAGAGAGATAACAGCTGCTTTCTGAATAGCGCTGCGTGGAGAAGC
ATCGTAATTCTCAAGCGCGGCAGTGAGTCCAGATTTTTCAATACCTAGGATACGATCGAT
TGATTCCATGCCAACATGCATGAAGGCAGCTGCTTGTCGAGAGATACCATCTTCACCAGC
TTGAAGTAAAAGACCTTGGAACTTTTCAAGTGCTTCTTTAGCATCTTCAATTAGCCCTAA
TTCATCTTCAGCTTGAACAATAGTTACTTCAGTATCTAATGCAGTTGGTTGAATAGCATC
ATCTGCATCAAGAACTTCTTCTACGGGATTATTTTCAATCTGATCTACTTCTGCTAAAAC
GGATGATACATCAGCAGAAGTTTTATTTTCAACGACTAATTCAACATCACCATGAATCGT
AGTGCCTTGATCTTCAATTGATTCAAGTGATAGTTTGTCTAGCATTTTAAGTAGAGACAT
GTTAATTACCTTACCTGGGAGGGATTATTTACTTGTTCTTGCGTAGGTCTTTGAATTGGA
CGCTGGTAAGCTTTTAATTCTAACTCGGTCACGAATACTAGTCGACCCATTACTCTGACT
AAACCAGCAAGAGCATTACTAAAATCTGTTACAACGCGACTTACATTATTGATCATTAAT
GTAATTGAATTAATGATATTTGAGATAGCAGGGATGCCATCAGGTGCATCAGGATCTTGA
ACTTTTGCTTGACGATTTCGCATATTAGATTGTTGTGCCCGAATCCTACCAGCTTGTTGA
CGATTAGTCTCAGCTAACTGCATTAACCTACCAAATTCAAAATCTAGTTTAGTTGCATAA
TCTTTTCTAGCATCTATACGTTTAGCCATTTCGCTTAATAATACTAACCGTCTTCCAATT
GAACTCGTATCATTAACCTTAAAGGTATTGTTACCTTCAGGTGCATCCTTCATAGTAGGA
ATAGTATAAAACTTAAAGGCAAAATCACGGACAGTGTTTACTAAAAAATCCCAATCCCTG
ATCTCTTCAGTCTGTGTTTCAGTTGGCCCTGCACCAAATAAAGTTTTATTACCTTGAACT
GGCATACTACGATAAACTGTAGAACTTACTCTAAATCTATCATCTTTCTTAATAGGCACT
GCACCCTTCATCCAGTTCTTAACATTAAACTTACTAAAGTCTTGTGCAGCTCTATCGGTC
CATTTTTCATCAAATGATAATTTCCTAAGGATATCTGCCACAGCTTGGATATATAAGAGT
AGGGATTTATCATAGAAAGCTATCAAGTTGTTAACTTGCATGAGTATCGGATCAACTGTA
GTCGGTTCGAATTTACCATCAATACTAAATCGTTTAGTATTTTTAAATTTTAATTCTTGC
TTAAATGGCTCATGCTTAATATTTTCAGCTCTTTCTAAAAGCATGTTTATTCGTTCAAGA
GTACGCTGATTACCTTGTGTAATAACACGTGCATACTCTTTTAATTCATCTCGATGCTTT
GCTTGGGATTTTTTCATTTCATCCCAGGTATTTCGTGCTGTCTTAGATAGATCTTCTAAT
GATACATTCTCAGTTTTTAGAATGTGTTCACTAACAACATCTTTGTTATCTGCATATTTT
TGAAATAAGCCATCGGTATAGGTTTTAACTAAAATGTCTTTTTCGGTGCATTCTAATCTA
CTTTGAATATCTTCAATTGATGGCAATAATTCTTTTAGATATCCATCGCATGTAGCGATC
ATATCGACGGGGCTGTCCCCGTCGATTGATTCAAGTAAGACCGAATGACTAAGTGAATTA
TTAGTCATAGATAGTCCTCAGTCCTTATCTATTACTTCTACAGATCCAAATAGTAGATGT
AGGGTATACATTAGATCATTCATACCGTTTTTGTGTTTAACCCAATTCTGGATCATTGAA
ACGGTATCTAGATCTAATTTTAACTCATTAAAGAGAGTAGCAATTTCGGCGCGCGGTTCA
ATGAGTTGTGCACCAGCATCTGGTTCAGTAGCTAGTAACATTGGCCATTGAGTAATTGAA
ATACGTCGATGACCCGTAGCAATGTATCGAACAGTATCTAATAGGAATTGATAATTATAA
CTATCGATATTACTATTATTGTCCTGTAGAATAAACCAATTATAGTTTCCGCTAAAAAGA
CGAATAGCACAAGCTAGAACTTTCTTATGGAATTCCCAATATGCAGTTTGTGCAGGAAAG
TTGGTAAAGATAAGATATAGTTCTTCAACTTGAGGATCACCGGGACGAGCAGTGAACCGT
GAAATGGCCTGTTGTTGACCACCTGAGTAATTACGACCAAATGGGCGTACAGCATTAGTT
GCTGGCATATCAATGCCCTCCATTAGTGACCAAGAGATAGTAGATCACGATGGTTAGCAA
AGAACTCAGATGCGCGATCTTGGATCATACCTAGGCGATAGTTATAATCAGAGATACGAA
TTTCATACTCTTTAATAAGCTTCTGTAGGCGTGCATCCACTTTACCATTACTTTGCATTT
CCCGCATTTCTTGTAAACGTAGTTGAAGTGCAAGGAATTCTTGCTCAGCTTCTTTAAAAC
GTTCAACCTGATACTCAGCTAAGAACTTGCCAAATAACACGAACGGGTTCTGACGAGGTG
AGAAGCCATCCATGTTAAGTGGATCACGTTGAGCTATAGAGAGAGCACTCACAGCTACAT
CATAAGTACCTTCATTGACTTCAGCACTAGATGCACGGCTTAAGCGTTGTTTCAGTTCAT
TAGTCTTAAGACTCATTGCTGGATACAGAGTAATGAATTGTGCCATATTGGTGTATAGCC
ATTCTTTCTCAGCACGAGATGATTTTAATTCTGTAGCCTTACCTAATTTCATTGCTTCTT
GTGCAATTAGATTTAGTAGTAGTTTACGGCCATAACGAATATAGAAACTAAGACCATCAA
CAAAACGTAGCATATTTGCACGGTCAAAGGTTAGATTAGAGTTAGTAAACTGAATAGCAA
ATAAACGCCGGATTTCGCTTTCGATCATATCAAAGCTACCACGAATATTTGAAAGAGTAT
CCAGTACTAGTTTAATCGGTTCAGTATTAAAACTAATCGCTCGCCGTAGTTCAGTAGATA
ACCGCATACCTAAATCTGATTTAAGTTGCATATTACCAAATACTTCACCTACATCGTCAA
TAATTGGTGCGAGAGTTGCATCATATTCTTCACGTAATGAACGGATTTGATTTAATAGTT
CACGACGTTCAGTAACTGGTGCGAGTTCACGTACATAATCAATAATTCTCATCACTCAGT
CCTTAAATATTGGTAGACATGGTTTTATTCATCATTTTAAAGATCTCAGTGATATCCGGA
CCTTTATTCTTTTCAGCCATTTTAATTTCATCAAGTCGGTGAGATGTAGATAGATCAACC
CCACGATGATAAATAGTTACTCGCTCATAACGGTCATCAATAACAACAAGCATCATTAGA
TAGCTATTGTCAAAGATCTTTTGCCGAGTGGCGAAATCATCAATACGACCATAAAGAGAA
TTAGAAGCAGCTTTAAGAGTTGACGTTGATACAATCGCGATGTTTGATGCATCTGCTAAT
GAAACTGTGCCACTACGAACAGATTGTTTCCAGTTGTTACGGCGACGATCGGTGATTTGT
TTATAAACACCTGACTTATCATTCATTAGAATGCGATAATGTTCATCGATCATATCTTGA
CCTAGAACGAAGTCACGCCAGAATTTAATTTGACCAGTTTTTACTAGAAATAGACGATGT
AGCCAAGTATCTCGTCCACCAGCACTAAAGATGTGCGCAATAATCTGAGGACTAACAGCG
GCTGGAATTAAACGGATCATAACAGGAATTTTAGCTTTCTGCTTTCCTGATTTAACATCA
ACGTTAACCAGCTTACCAACGGCAAGGTTTTCAACTTCATAAATCTTTTGTGTATCACCA
CTAGAAATACTTTGTGCACTTGTACTTTCGTCATTCGCTTCCATTGAAGCTACCCAACGC
TCATGTGAGTGTTCAGCGACGATATTTGGTAAATGCACAGGCTTCTCAAAAGACTCTAAT
GAAGGAAGACCATCAGAATACTCTTCGGCAGAGAATACACTAGTGCGAGTTACACCTGTA
CGGTTAGGATTAATAGTATCGAATACTTTTAAAGTATCAATCTTATCCACACCTAGAAGC
ATATTAACAGCTTGTAGATAATAACCAGTAAAAGACGATGAAACAAATTTCATAACGTCT
TTCATGTAAGGTTGATCAACTAAGGTTTGGTCTACGATGACCAATGGTTCAACACGAGTT
GGACGTGCTAGATCGGTTAGAGAAGAAATCTCTACACCATTTTTAGTGGCTTGTAGGACA
GTCTGCATTAGACCACGTACACCTTGATCAGCAGCAACAGTCATCAGAGCAGAACTTACT
GCTCCGGCGATTACAGGTAATGGCATTTTTCTCTTCCTATATGGGTTTGCAGAGAGTTAT
AAAAAATGGCAGCATCGGAATTTTTAGATCAGATCATACAAAACATTGGTCAGTCGCCAC
GTGATAAAGCCATCTCTGATTCTATCAGGGGGCTTAATATCATTGGCCGTAACTCGACCA
TTGCCCCAAATACAGAGAACCATGGTTTTACATTCTTTACCAGGCCGATACTTAATTTGT
CATACGATAATTGTAAGATCGATAGAAAGCTCACAATGCTATTAAATGATAATCCTTATA
GCTTATCAAGATCAATAAGAGCTATCCTAGATCCATGGTCAAATAGGGATGGAATGAATG
CGGTGGATGTTGATCCACTTAATCCATTTATTCCACTTTTATCAAACAACCTAGTTAGTT
TAACAGGTTGGGAAGATTTCCAATTAAACCTAGCAACAACTAACCCAGGTATATATCGGG
ACGCTATGTCATATGTAGATGATGTCCCGTATCAATTTAACACATTTGATTTGCAAGCAA
GCTTCCGAAATATGGAAGGTGACCCTATCACTAACTTGTTATATATGTGGGAGCGCTATT
CTGCATTAGTGAAAGAAGGTAGGTTATGGCCTTATCCAGAGATGATCTTACTAAAGGAAG
TAGATTACAATACTAGGGTATGGCGAATAGTCACAGATGTAACTCGGACATATATTACCA
GGATATTCTGTTCAGGAGCATCTATTTTCATGAATGCTAATACTGGACGCACTGGTGACT
TTATTGGTGATGGTAGTGAAACACCATTCCAGAATGCGAATGATTTACTATCTGTATCAA
TACGATGCATGGGTATGGATGTTTATGATTATATTCTGTTCTATGAATTCAATGACTTAG
TTGAGGACTTTAACCCCAACATGTCAGATGAACGACGATATACACAGATGCAAAAGATTA
AAATCTATGAACGTGAATATTTTAGTAGATCAAAAGTATATCCATACATCAATACAGATA
ACAGTGAATTAGAGTGGTGGATATCTAAAGAAGACTATAACGCCATGTTACCTGGTCTTT
TACGCAATCCCCCACCACCTCAGAAAGAGGGTTAATTAATGGCAAGTGAATTTAAAAATC
AGTTATTAAACCTGAGACGAAATCCAGTACTGATGCAATCATTAATGTTATCTGAACTAG
ATAAACAACTTAATGGTGAGACTGGGGAACGTTATGATATTCCTGATTCAGCAAACCCTT
TCGTTTCATTGATGGAGATGGGTGTTATCGGAGTATCGGCTGCATTTACTGAAATGGAAG
CTTTAAGTAGGCGTCTTTATCCACGCTTAGCGATTACCAGTGATGAACTTTATCTACACA
TGTCCGATGAAGATTATATTGGTAGATTTGCTATCCCAGCACAGAACTATTTTAACTTCA
TTTTAGATTATGATGAAGTTATTAAGTATGCTGTTCCTTATGGGGATCAAGGTCAACGTA
AATTAGTTATCCCGCGTTTAACTAAATTTACAGGGGCGGGTATATCATACACCATGCAAT
ACCCTCTAGAACTGCGTGTAATGCGCCATGGTGGCGTTGTAGCTGAATATAATACCGATA
GCTTATCTCCTATTGAAACGCTCTCTACGAACATTGCACAGATGACTATGATCAATGATG
GTCGTGGACATCGATTAATGAATCTGATCATACCTGTTAAACAATTTGATATTACTCGAT
ATCAAGAAACACTTAATCCATCTAATCTGTTTGAGAAAGAATATGTTTTCTCCGATCAGT
TTTATTATGCACGTGCATATATTAAACGGACAGTAGATGCACAGGAATGGGAAGAAGTAA
TAACTACACATACTGATCAAGTATACGATCCTCTTAATCTAACCGTAGTTTTAAAAGTTA
TTAATAATAGGTTACAAGTTAGTATACCAGCTATTTATAATACTACTGGACTAGCTACAG
GTGTAATTCGTATTGATATCTATTCTACTCGTGGTAAGATTGAAATCGATTATGGTAGTT
ATCAACCTGACCAATTTACGTATAGTTTAAATGATATCGACGATGATCCAAAGTATACTG
AACCACTTAAGAAACTTGCTCGAATAAGTGCACTTAGTACTTATTATGTATCTGGTGGTT
CTAATTCAATGGACTTTGATACGCTTCGTAACCAGGTTATTGATAATACCACTGGTCCAC
AACAGTTACCAATCACCAATGTACAATTAAGTGCTGACTTGGCTAGACGTGGTTATACCC
TTGTAACTAATATCGATAACATCACAAAGAAACAATATCTTGCTAGTAGGCGACTAGGTG
CTCCTCGTACACTTGACATCATTTCAGGTGCAGGTGTAGCAATGTCTCAGCTACAAGTAT
CGATGAATGAATTAGTTAAATCTAAGCATGTTAAAGATAATGGTGATAGAATCACTATCC
TACCATCAATGCTCTATAGGTTTAGTAATGGTCGTGTATCAGTCGTACCAGATGCACAAT
TAGATTCTATTTTAAGTAGTTCAGCAGAATCTATTGCACGTCAAATAAACGACTCACGTA
CAGTTTATTGTCCATTCCATGGTGTACTTGATGCAACGGAAGATAACTTCGATTATCGCC
CATACTACTTAGATAATCCAGTTATCAATGAGAAGACTTTTGTTGGTGAGAATGATACAG
CTCAGTTACAAGCTAGTATTGATGTATTTGAAATTGATCGCGTCACTGAAGGCTATCGTA
TTAAGATTCGATTAGATGAAGGCGAACAATTTAAGAAATTAGCTGATGAGCAAATTGCTG
TTCAGATTGGATATCAGCCTAAAGGTGAAACAAAGTATGCATCAATTAATGGTAAGTTCT
TAGGCATTGAAGAAGAAAAGCGTGTCTATGAGTTTATCATTGAAACTAATTATGATATTG
ATTCAAGTGGTGCGATTTATACCACTAACATGTCAATGTATGATCAGTTCCAGAAAGTAT
TCTCTACTCCTTTAAATACTGATTTTGATGTATCAATCATTGTAGTAAATACAATCACTC
CAGGTTATATACCAAATGAATTAGACTCATTGGTTCAAAGGCATCTACTGCCATCTGAGT
TCATGGTTATTAATAGGGAACGGTTAATTACAACGCTTGGTTATGACATGACTAAGATAT
GGCACCGTAATAGAACTGTTCTTAGCCAAGAATCATATCAGACCTGGCCAGCAGATGTAC
CTGCTTACTATCAGAAAGATGTATTTGAAACTGATTCAACTGGACACATTGTTATTACAA
TAGGACCAGATGGTAAACCAGTTTACAAAGTTAAACATAAGAAAGGTGATCCAGTTCTAG
ATGCTAATGGTAAACCTGTAATGCAGTATCGAAAGGGTGATACTAAGTTAGATGCTAATG
GTAAACCTATTCTTCTTGAACCACGTAAGATTCTACGTGAAATGATGGTACTAATGGTAG
ATGGTCTATTCTACTTTGCAACTGAACAGGAAGCTGTAGCTTATCGCAAAGAGATACCAA
TGGAGTTCGTTAAATGGATCCAGAATGATATTGATTATATTGATCAACGTCTATTAGAGA
CATGTGAACTCTATCTATATCCGACAACTACATTTGGCGATACAATTGTTTCTGTTAGAG
AGGGTCAGAAATCAACTGTACAAGTTGATCAGAACTTCTTTACAATGTTGTATCTAAAAC
CATCTGCATTTGCTAACTCTACTATCAGACCGTCATTAATTAAGACAGTTAAGACAATTG
TTAATGACATGCTTGGTCGTAAAACAATTGCATTATCTGAAATCATTGCAGCTTTAGAAG
CAGCTATTGGTGAGGATGTGTTACAGATTGATGCTGGTGGTTTAGGTGGTTCTAATAACT
ATTCCATTATTACAGTAGATGACGATGCAGTTAGGTTGACATTACGTAAGAAGCTTACTG
TTATGGCTAACCAGGAACTAACCATTGAAGATGACATGTCTATTACTTTCTTAAGACATG
AGCAAATTCAATAAGGTCATAAGCCCTCCCCTAGGGGAGGGCATTATGTTGTATTTTACA
TTGCATAACCACGTTTAAAGTCATCGATGACTTTATCAAATCGACGCTGCCATTCGCGAA
TAGCATCATTGAAGCCATGGATCTTATAATCTTCACTAACTACTTGTGCACACTTACCAT
AATAGTTAAGTACGCAACTAGCTGCTAGATATTGTGCTTGAGAATATTCAAACATCGTAT
CAGTAATACGACGCATACCTTCAAGGGAAGTGTTGATTAATTGAACAGACTCACCAATAG
CATCAATAGTTTCTTGACTATAATTACGTGATAGTTGCTTTAACTTACGTAGAGTATCAC
TGCTATTGCGATATGCTTTTAACAAATCATCAAGTGGTGCGACAAAACGATCTTTTAACA
GATGTGAGTTTTTCCAGAAATTCTTATCAGGGAAATCTGGAATGCTTGAACGATCAGGAT
ACACTGTAGATAGTTCATTGCACCGGGTAGCTAGTTTACCAATCTCACGGATATTAGCGA
TAGGCATAAGTGCACGATAATCATCACCATTGTAATTAACATATTCAATGATAGTGCCAA
CTAGCTTTTCCATTGTACCTAGTTCAGTACGTGCTTCATTATTAATATTAATGATTTCTT
TCTGGTAGTATACACTACCAAATGCAGCAGCATATACACGACTAGCTAAGATTTTAGGAT
CATCAATGATTGATTGTGCAATTGCATCAAACTTTTCTAGTAACGCATCCTTTCGATTAC
CTAATCGATTTTTAACTAGTAACTCTTGAAAGAGAGTACGTGCTTGATCAACTGCCCGGA
CAATCTTTCCATAACGAGTTGCTACAACAACATCGCGCTTCTTAAAATCACTAATCCAAC
GTATCAGCTTAGCAAATAGTTCCATTAGTTTCTCAAACCAAGTCTTAAGAGTATTAATGA
TGGTAGCCATCACTGCCTCTTGAGAAATAGTCACATTAAGAGTACTACGGGTTGGTGTAA
ACATAGAAGCATATTGCTCAAGTGAAGCTACTGCTTTCTTAGGAGCTTCAAACCCTTCAG
TGCTCATGCGCTTAACTAATGCTCGAATACCATCAACATCTGGTTTTGATATACCTTCAA
GTTTAGTGATGGCATAAATATCAGATAGCTGATGAATCGCTTCTACGGCTACACACATCT
TAGCTAAAACTTCAGGAGCTGAGGCACTTGAAACAGTTTCTTCTTCACCTTCAACCATTT
CTGGTTGGTTCTCATCAAAGAAAAGTTTTTCCTCTTCGACATCTACTTCTTTAACATGAG
ACATGGGTTTAACCTCACTCATCAACGCTACCGATTAATTGATTTAATGCATTACCCATG
TCTGCTTGGATACGGTTATAATCTGGGCCATTAACAACGGCAGAGACAATATTGACATCA
TCAACAGTTAGATAATCATTAGCTGTATCAGATGTTAGTACAGCTTTGACAAGACATGGA
TCAGTAATACCGGTGATAAGACTTTGGCGAATGCGAAGAATATTGTTAAAATAGGTTACG
GCTAGTTTTGTATCAACTGCAATATTCTCATTAACTTTACTTAAGATTTTCTTAAATTTA
GCAACAGCTTTATCCTGTTGTTCATCGAAATCAAGTAGAGTCTCAAATGGAATCTGTAAT
GCCACCGCATTAGCTAGGAGCATTGCGCGGGTTTTACGATCAGAACCACCTAAGTTAGGC
AGTTGCTTTAGAATACGGGAAAGTTCAAAACCTAATTGTTGCATGATTTTTCTCTCTATT
TAGTTAAGCGGTAGGCTCTTACAAATAAGTCATTATTTAATAGACTTTCTAAAGTATCTT
CTAATTCAGTAGCCCGTTTATCTTTACGACGCTGGGGACTAAGGAAATCTAAAATTTTAT
CAAAAATGGTCCACTCATTGGCTAAATCGTTTATCAATGAATCAATTACTTTAATGTCAT
CATCAATTCCTGGTTTTAGTGATGAGTCCTTTACCCATTTAAGTTGTGCTACTAGATCTT
GTTTAATTTTCATTAAACGTTGTCGTTGATTGTCATAGCGTGAACTAATTGGATCATATC
CAACTTGTAAATTACCGGCAATGATACCTAGTGTAACAGGTAAACCAATGATCATAAATA
GACCTTTGAATACCTCAGCAGTAATATGACTAGAAACAGATTTATATTCACGGTCATTAT
AATACCGACCTGATTTAGTTAATGCCGTTGCCAGTGGCCTTCCATAACCTAATCGAGTGG
CGAATAAATCAGCTAATTGTTCATCGCGTTTACGTGATTGAACATCACCAGTTAAATGTG
GACGTTGTGCTTTCTTAAATGATGATAGAATGACTTTACGAATATTTCCTTCAGTCCTAT
CATTAGCAAACTTCTCATAATCAGCTGGATCAACGTTCTTCTTTAACCATGTTTCATCCA
ATAATTGAATCTTATACTTATTAGCTTTCCTACCCTGTAAGATAAGTATGCCTTCAGTAA
GTGCATAGTTAAGATAGACGTAGTCGCCCATTGTAGCTAGTGTTTCCCAACAATGCCCAA
TTTCATGTAATGTAATAGCGGTAGCTTCTGCTATACTAACACGGTTAATAGCCGATAATA
GACCAATAGATAAACCGATCTTGTATTTAAAAGTTGAAACAGCTTCACCTGTAACTTTTA
AATTTGTTAAATCAATATTAATGAATGTATTGACACCTTGCTCAGTTGCTAAAGTAACTC
GATTAATATTGTCAAATTTACTACCAGTATGTCCAACTGTAAAGGATGTGAATGCACCAC
CCATTATACCCTGATCATCTAGGTGTGTAAATTCTATATTCATATTGAGGATCTTAGAAA
CATCATCACTCAGTGACCTAAAATCATTAATATCTATTCTCTGTTGATTATCTAATTTAT
CACGAATCTTGGTTAGATGTTCTTCTATCAATGGACCTAGGTCACGCTTAACATTCACAT
CTAACACTTCATTAGAGATTTCTATCGTATGTAGCATTTTATATCTCTACCAGTATTAAT
TTATAATCATAAAGATAAGGCTGCGTTGTATAGAATTCAATCTTATGCACTACGTATTAT
AAAAGAGGCGCACACAAAATGTTTGAGTTATTGTTGTCTTCTGGACTAACAAATACGCCT
ATATATTCCGATCCTTATCCAGGTCCTAAAACACTGATAGCTGGGACAAGGGAATTAGGT
TTGTATGGTGAAACAACATCTTCAGAATTATTTGGTTATGATGATGCAGCTAAATTAGTT
AATATGACATTTGGAACAGTAATCAATGATGGACAACCATGGTTAAAGTTCTCTAATAAA
AATGTTACTTGTTACATACCAAAGAAACCAGCTCGTAAATCAATACAAAAATCACGGCTT
AATAATGCCGGACTTCGTAATGGTGACTATGAAGTTAGATTTATGGGAAACATTTATAAA
TTTGGTTTATTAACAATGGTTGGTGCCACATCAGAATGGCAAAGGTTACTTTATAATGTT
CACGTATCTAATTTAAATGGTGCTAACTGGCCTTATAAATTTACAGATAATGACTTAGGT
ATTAACCAAGCAGTCGCCGGTGGTATTAGTACTGATGGTACTACTAACTGGGGAAGAGAC
CCAGGTAGTGATCCAGTTAATGACCAGCTCTATCGTGGCTACTATGCAATTGATATAACT
AACTCGTCAAGTAGTGAAACTGGCGCTGCAACCCAAGGTTGGCGTCCAGTGGTTTATGCT
AGTGGTACATATCCATTCGTTAAAGATAATGTTATTAAACTTACTGATTTTGAAACTGAT
AATGAAATGGCAGCCGGTGCCCAAATAGGTAACTTAGTCTATTATTACGGTGGTAAGAAT
AATGCTAATACTGCTCTACGTACACTAGATTTAGTCACGGGTAAGTTAACAACATTACAA
CCAACTGGTACGCCTATAGCAGTTAAACAATGCTCGGCTGTTGCATTTAATGGTAAAGTA
TATTTCTATGGTGGTGAAAGTGCCACAGATGGTGTACTTACTAAAGATATGCAGTGTTAT
GATCCTGCAACTAATACATGGGAAATAAAATCACCAGGTACAATAGCTTGCCGGTATGCA
CGTGGAACTAGTCTTAATGGGTTAATCTACATATTAGGTGCAGCTGATACAACTGCAACA
AATAATCGTGGATTTACATTAATGTACAATCCAACTACAGATACATATACCAGTGGTGCA
GCATTTAATGATACTGGGGTATATACCCAAGATTATTCAGTGAGTGTTTATAATAATAGC
GTATGTGTTATTGGTGGTATGGTTGGTGCCGGTGTTAATACTGGAATCATATCGTACAAT
CAAACCAACAATAATTGGTCTAAACCAATAACTACAGGTATACCTCTTGGTGCAGCTATT
AATATACGCCCATCTGGTGAACTTTATGTAATCGCTGGTGAAAATCAAAATAATGCACCA
ATTAAGTATTATGAACCACGATTAGCACGATGGATAAATTTAGGTAATTTAGAAACTGGT
TTAGTGGATGCAGGTTTTGCCAATACGTTTAATAACGATGGTAAGATTTATGTTCTAAAC
GGTAATCAGAATTTAAAATACCAAATTAGTTAACGAAATAAACCCTCCCGTAATGGGAGG
GTTTTATGATGTGTCGAAGTTTATATTAGCAGTAATGATCAATTGATAATATTTAATTAG
GAATACCTTCCATTTTTAATATCCTTCATTGAGTAGTTCTGCTAAAGCACGTGCCTGTGA
AATATCAGTAGAAGCAAAAACATCAGAAAGATAGTTATCATGTTCTTTTGCATTCTGACG
TAATTCATCCATTAGATGTTTATTAGATACAGCCCATACTTTCCACAGATTTTTATAATC
ATCATATTTAGGATTATATCCGTCTAGTGGTGGTTTTCCTTTACCAGCTTTCCAGTGGTT
GGAGTATTTGCGATATCCTTTACAATCTAACTGCCAAGCTTCCTCAATTGTTCTGCCATC
AGGAAGCCTTGCGTAAAGAGCCGAGAATCTTTTGTCACCAACAGATGAACATTCATATCC
TCCTCGGCGCTTCCACTGATATATTACTTTGGGTTCTTATAGGTATTAATAAACTTCTCT
AGTAGGGTATCCATTTTAGGAGCGCCCATGATCTTCTGTGTGATTTCAATAGCATTAACC
CTACCGCCTGTTAAGTTTACGCCAACTTCGCGGCGATGGTGTTCACTAATTCCTTCCATC
ATCGAGAATTCCATTACACATGTTTCACGTGCAATATTTAGAATCTGACTAACGTTAGGA
TATGTCGACACGATGTCAAGGTCGGCGATATGGCAGAAGATTAAGGTTTTATAATTTGGT
AAATCTTTAACGCACTGTAATCCATTTGGACCTGCCATGTAAGATGGCAATGTAACAATC
CAGTCGTTATGACCAATAACATACTGATCTAACTCATGTACCATCTGATCAGAACTCGAA
CCAATAACACATGGATCTGGTCTATTTAGATACCAGAAATGCATGTCATCACATAGTCGT
TTTGGGTTAGAGCTAAAGTTCTTATAATCGGATGCTTTAGAGAATAGACTTATAGAACGA
CCAAGGTCATTTACTTGTTCATCAAGTTGTTCTAGTCGAATGGAGTCGATGATGTTATAA
AGACCATATTTGATTTTATGTTTAGTCTGCATCTCACGGTGCCAGTCGATACCACCAAGA
TGTGCAGTTTCTTTAAAGGTTAACTTACCAAATTCCATTACATATCTAATAGAATCACCA
TAGGTAAAGTTATTATTTAATATAAAATCATCCACCGGTACCCATTCATTACTACCTACA
TTTTTAATATATTTAGTAATAACTCCGCCTGGCCTGTCAACTAGTTGTTTATTAAAACCT
TCAATAAAATCTTCTAAGTCCTTATCATTTTTAATGGTCGTAGATTTATCGATGTTTAGT
TCTTTTCTAAGAATAGACTCTAATCGATAGTTAGGATCACGTCCTTTAGCTAATCGAGTT
ACTCGATAAGTTGACATAGAATCGATGCATTGAAAACTAGAGGGTGCTGTAATCCAATGC
CATTGATCCTCCGGTCCTTTAGATTTGGTGACCCCAGATGCAGTGGTGGTCATAGGTTCG
TCAATCTTAAAATGGAAATACCTATAATTCTTAGGTACCGATGGATCAGAAAAAACATCA
GCTGGATTAACACCATAATGTTCTAATGTTTTTAAGATTCTACCAATGTCGAAGCTAATA
TTCCAGAATGCGAAGAAATCTGGTTTCCAGGTATGTAGTCGTTTAAAACATTCAATAATA
ATATCTACTGGTGTTTCAACAACCATTACTTCAATATCTAGACCACGTTCTTTAATTAGA
TGCGATAGCACCTCTTCTTTTTGTGCTTGTTCTAATGTCTCTTTCACTGGATCTTCAATA
TCACTAACCCATCGTTTTAAATAGGCTAAGAAAGCTTTGTTCTTATGTGTGACTGACATA
CAGATAATCTGACCATCGTTCTCAGCTTCATAAACGTTGGTTTCGATGTCACCACCAGCA
ACAGTGTTTAGAGAGATTAGACCAGGATAACGTTCTTGATAATCATGCTTCAGATAACAG
GTTGAACTAACATCACTGCCATATAGGTAAGGACTACGCGCTAGTTGACGAAGAGATCTA
GCCGAATAGTCACCTAGGATTTTACTAATCTTACGTTTCATGTCAATTTGAGTTGACATA
TATTTAACTAAGTTACGCTCATATTCGTAATCTTTCTTTTCTTTATGGTTACGCATTCCT
TTTGAGGTAATCCAAAAAGGACGCTGGTAGTTTTCCCACATCCTTAAATTATTTGCAGTT
GTACCATCTTCAAAATGAATTCGTTCTTTAACGAAATGTGCATCTGTAAACTTACCAAAT
TGATCAGTACAATAAGTAATATGTTTGGCTTCAATGCCCTTAATCTTTTTCTTATTTGTA
ACCTGTTGTTCATTAGACATTGCTATAACCTCAGCACATATTGATATTTATCTACGTACT
ATATTAGTACCTAGTAAAAGTAATAATCAGCATAAAGCCTCCCCTAGGGGAGGCAATATG
TTTACATACCAGCTTTCTGCCGGAAGAAGTGTAGAACGAGTTGCCGTTTACCTTCAGGGA
TGAAACGTGCAAGATAGTTGATATCTACTTGATAAAGCGCATCTGATACATCACTAGTAA
ATAGGCAGAATAGAGTAAGAAGATTAATGTGTGCTTCTTGACGGTTATTCTGAGTAGGTA
GAGTAGCAACAAAGCGAAGTGCGTTAGATTCACTAAATAGGGTATTACGATTTTTCTTAA
CGAAATCAAAGATAATATCCATGCCTGATTTAAAGCCTTCAATATCTTCGATTGTCATTA
CGTAGTTGAATACATCATCTAGTTTCTTTTGTGCATCTCCACCACGTTCGATAGTGATAT
AGTCACCAGGTTTAGCACCAGTGGCAAACTGCTCTAGACGAGATTCGATAAATACTTGGT
TCATTTTGGTCATACCTTTACGAAGTACAGGTTCATTAGATGTTGATTCACTATTCAAGA
TTTTAACTTCCCCGGTTGGTTTAATATCATCTTTACCAGAGAATACTTTCCATTGTTCAA
CAATAGTTTCAATATCTTGACTAGTGGTAGGTAAGAATAAACGATCGCGTAACTCAATAG
CAATTTTAAAATCTTTTATCAAATTACTATTTTTAATAAGTCCTTCAGCCCAGTCTTCAA
GCTCTGCTGTCGTCCAATCGGTAGGTGGAGCATTATCACGAATAACACTATTAACCCAAC
TACCACGTAGTGTTTTTGGTGGAGTAATACCTTGTTTAACAAAATCAACCACTTCTTGGG
TAGACCAATCTAGTGGAATATCACTAGTGTGAATACGCAGTTGCTTGACTAATTCTTCAA
GCTTGATACTGTCTTCAGTATTGCGAATATCTTGTTTCAGAGTATCTTCATTAACTACCG
GTACAATTACTTCCTCAGTAGTCTCATTCTCTTTAGCCCGTTTAGTTAAAATATCATCAG
CAATATCGGCAATATCATTAACTACACCAGTTAGACGATGGTTAGCAGCATCGATTAGTT
CATCATCTGACCATTCATCATAAGGACGATATTTACGGGTTGGGTCAAATACAAAGCTAC
CGAGTTCAGTTTTACCTTTAGCAATGCCATCATTGGTAAACCATTCAATGACATTTTCAT
CTGTCCATGTTTCAGGAATATCGAAGTCAGTACGCAGTGCATTTACATTAACTTCAGCTA
ATTCTGAGGTATCGTTATCTGTATTAACTGAAGAATTAATATCATCTGCTGTAGCATATA
CTTCCTCAGGAGATACTAATTCAATATTTGTTAATCCTCGACCAATTTCAGAAGTTGGCT
TATCTTCAGTTGCTTCTGATAAAACTTCGTCTTTGACAATGGGTTTTTCAGGTAAACGAT
TGAGTTGAGGAAACTTTCCAAATCGTTGATTCTTTTTAGACATTACTAGTTCCTTTCGGG
TTATAATCTAAATAATGGAGAAGCTGAGTAACTTCTCCATCTATTTTAACTGATGCGATC
GCGATAAGACCATTTAACAGTCTTAAGAACAGCACTGTGCATTTTAGACATGAAGATGGA
TAGGAATCGACCACCAACACCTGCTGCCGCTGCCGGTATGCCATTAGGGTTCTCTGACAT
TTGTTTACCCATACAGACAGCGCATACGTTCTTACCTCTACCTATTACACCATTAGCTGG
ATCACCACCGGCCTTACATGTCATTGGACCACGTAGTTCAATCTCAGTGCCAACTAGTTC
ATCAATATTAGTTTCTTCAATAAGGATAGATTTCCCATTCTTAACATAAGACAAGCCAAT
GAAATCTTTCTTATTAAATTGATGAACATTGGTTGGAATACCTATCCATGTATTGCAGTC
TTCCATAATAATCCTGACTGTACCAACCATTCGATAAATCGTTTTAGTTGACTCACCGCC
AAGGGCTGTTTGTGCACCACGGTTATATGAACCATAACGTAATGAGTTATTCATTACTGG
CATATGGTCAGTGTCAATGCCTTCTTCCAATGATTTACCGATATATTCAACTGATGTACC
ATCTTGGAATGGCGCTTCCCCACCAAACATACCAAATACTTTCTTACGCACGCCACCAAA
TAATTTACTATGTTTAGTGTCATAGAACTCATAAGAAGGATCGTCTTTTAGATAGTCCTC
ATCTAGCTTGATTAATGCATTACCCATTTCTGCAATAACAGCCGGATCATCTAACCGATC
TTTATATTTCTCTTCTAGTTCTTTACGTAACTTATCTCTGTCTGGTGCAGAGGTCATTGA
TTTCTCAGTAATGGATGTTACCAAGGTACTGGCATATGCTGGCAATGCTAAACAATGATC
TGCAAACATCCGATATTGCCATACATATAACTTACCATCAGTTGCACGCGGTGGATTATC
ATCATCAATTGGATCATCGATCATGCGGTTAAGAATTTCTTTCTCAACTTTACCAATATC
GAAATAACCAGATTGGAATGGGAACAGATCACCAAAAGGTAAACATAATACTAAATGGTT
AACAAAAACGTTACCATAAGTTGTTCTTAATGCACCTGGTCCTTTATAGTTTTCAATTTC
ACCAGGTTGTAAAATGAATTCATCTCGAAATGCTAATGGTTGTTTACCTTCAATATAATC
GTCGATATAAACAACTTCATCATTAGAATCGCCTTCTTTATAGAAGAACATTTTACCATC
TTTGAAATATGGCTTGTATAGATATTCACCACCATCATTTGGGTTATAAACGATATTAAA
TAAAACGTTAACCCATGCTCGTTTCTTATCTAATCCAGCATTGATACCCATTAGAAAATA
ATCACGTAATTTCATTAGTATCACCTATGCTATTTAAAATCTTTTTAGCTTCATTTTCCA
TAATAGATCGTTCAATATTGTTAGATGTGAAATCATCTATAATACCCATTACCGTTCTAC
TAATTTCATTCTGTTCAGTATTTGAAATTAATACCAATCCGACAATTTCTTTAGCAACTT
CAATAGGCGTCATAGCATCTAATGGGCCAAATGAAATTTGTAATAGGTCATCAATATTCC
TTTTACCAACACGTAATCCTGCATTAACTAGTTCCATAGCTAACTTAAATGATGCGCGCG
TATGAATACGAATAAGGCGATTAATTAAATTAATCCGTTCAACATTACTTACATTGCCAT
ATGCTTGACCACGGGTAGTAAGTACTGACTGAATAAATGATGTAATATTACTAATAAGTG
TTGGCGATACACTTTCAATTAAGTCAATGATATTTAAGAAATCTACACTTGTATCAATAA
GTTCTACCAGATGCGCTAATGTCTCTTCGGGTGAATATTCAGCTAACAGAATATCATTCA
CTTCATCTGGAATAATGTAATCTTCTAAACTAAGGAGTGTATTAACAACAGCTTGCCGCT
CAGCCTGAGTTGCTTCATCACTAATTTTAATAAGATGCTCATTTAGAATTTCATCTTGTG
CAATGCGAATGATATCATCAATGCGAGAAAGAAGCATTGCATTATCTGAGATACCTGATT
GAATCTGACATACTGTATTTAGTTCATCTAGAGCTGCAACATAATTAATCTTTTCAAAAG
ATGCTACGCAGTCTAGGATAACTTCTTTACGCTCTTCATGTATAAGAGGCGTAAGATAAC
TATCAATGAAATCTTTCATGACGATATCCTTTTTATACGGAATTATTAACATAGGGTTGG
ACAGATAGTTATTCTTTAATATAGTGTAAAATTACCAAATGTGTTAAATATATGTTGAAC
TAATCATTTTTTTCATTTGTCAATTTATATTGGCAAATATTAAATTAAGCCTCTATCGGA
GTATAACAAGATGTCAAATAAAGCTAAAGAACCAACTCTAACTAAAGAACAGGAAAAGAC
GCTACGTCTATTTGAACGTGCAGTTGGAGCATCAGCTGCCGCAGCTGATCATATTCTTAA
CCGCGATGGTTGGGATGTTGTCAATGTACTAGCAGGTTCTGTAGGTAAGCGTATTCTACA
AACTGAAGTATTCATTAATCCAGTTATTAATAAGATCAATGAAATTATTCCGACGTTATC
TGATCCTACTGGATTCCAAAAGAACCTACAAACAGTTATTGCTGACCTTAATCGACTAAC
TAAAATTATCAGTGTCCTATATGCACAACATCAAGGTAAGGTTGGTCCGGCAAATAATGA
AGATGAGCTAAAACTCATCGGTGAGCTTGTTACTAAATATACTGGTATTGAAGGTGCTAT
TGAACGCGGTATTGATGGTGTAATCCTAGCGATGATCGGCACTCTTCGAGAAGCTGGTTG
GGACGAAAAACTAATTCAACAATAATGGAGTTTTAAATGGCTACTGCTGACTTCGATAAT
GAGTACGATGATACTCCAGAAGAGAATCTACTAGATCAAATTGATGAGATCAATGCGCAA
CACGATCCTGACTCTAGTATTGAAAATAATGACGAGGTGGAAACTCCACCTTTCGTTCCT
GATCAAAAAGAAATTAAACTACGTATTCCACGTAATGCTAAAACTGGAAATATTCGGGAT
GCTTCTGAACCGGCTATTAAAGACAGTGCTAGCATTCTTACTTTGCTACGTGAGAACCCC
CAGACTGTTTATGATTTCCTGGATCACAATAAAGAACTATTTAAAGCATTTGCTGATAAT
AAAGTAGATGACACTAGTGATGGTGTAAAGTGGACAGCTGCTATTCTGGAGGGCTTAGCT
CACTCTATTGTTGAAGATTCGCCAATTGGTGCAACTGCCCGAGAAGGCTCTGAATGGTCT
AGTGAAATTCCTTATGGTGAAAAGATCATTCGACCTGGACGACCTGTATTTAATACCAGT
GGTCGTAAAATGACCGATGATGAAACTCTAGTTTTCCTAACACGTAAAGCTAACATCGGT
ACGCCATATGATACTTTCCTACCTCATTCAGGTATTTGGGTAAAACTACGTGCACCTACT
GGTACTGAAGTAGCTGCCCTACAAGCACGTCTAGCACAATTAAAGATCCGGCTAGGCGCT
GAATCAAAAGGTATGCTGTTCTCAAACATGGCAGCTGTGTTACTTAACATGGTTAGTAGT
TTAGCACTTGCATGTGTTATTCAATCCAACTATAAGTTCAACACCCCAACTGATCTAGAG
CATGTTATCTCAGTAGCTGATGAGCCGTTCTTGCATCACGCACTAGCGGCGACTATGTAT
CCAGATGGATTTAATTATAGTTATCCATGCATTGCAGATGCCAATACATGTAACCATATT
GAAACAGCGAAACTTAATATGTCAACGCTTGTTCAATATGATCTACTGGCATTCTCAGAA
GAACAAAAGAAATATATTGCACGTAAGTTTAATGCATGTACTGAATCTGATCTTAAGACT
TATAAAGAAGGATTCCGTATTGAAACTCAAGTAACTGAAATGTTTGGTGACGTTGGTGTT
GTTCTTGAGATGTGTACAATTGCTGATCGCCGAGAGTCTGGTGATAGTTGGATTAACTCA
ATTATCGATATGACTCAAGGTAGTTTTAATGAACCAGTCGATGGTCCGCAACGTTCTCAG
TATATTGCACGTTTACAAAAGATCTCTATTGCCCGTCAGTTCTCACATTTTGTTAAAGAA
ATTAAAGTACGTGAAGATGACGATGGTGAAAGTTCTGAAGACAATATTCGTACTGCAACC
ACTAATCGTGAAACTATTGAAAAGTTCTTAACTAATATTCTATCCACACCGGAATATAAT
GACCGCTTTATTAATGCGGTGAGTAACTTCATTGATTCACAACTAATTGGAATGATTGCA
ATTACAAGTTGGAATTGTCCAGTATGTGAGTCACCTGCTGCTACTAAATTTAAAGAACGT
TTCCCACACTTAATCCCTATTGACATGCTGTCAACTTTTTTTACTTTGGCGGCCTCGAAG
TCAAACCGGATGGTGTAGTTAATGATCTGAGATATAGTCGTCTTGAGGCCACTTTCACGG
AAGATAGTGGCCACATCCTACATCCAAACTTTGGTTATGATAAGTTAACTGAAGATGATA
CACACTTAGATAGACTATTATCGACAGTAGGGGCAGTAAGACCGTCTACAGCAGCACGTT
TAACAAGAGAAGCATATCTACTTGATTACGATATCGGTTCACCAGAAAACGGACCCATGT
GGTTAAATAAAGTAAATCCTAAAGAGGATTTTATCGAAGGTGGGTTGAGAAGGAGACTGA
CTAGAGATTTCTTCAAGTATAAAGTAGGTAGCTTATCTAATATGTCATTAACTGAATTCT
TTGAATTAGCTTATTCTGAGGTAGAATACATAATTGAATTATGTAGGAATATTACTCATA
ATGAGGCTGTTAATAACAATAACCAGTTAAATGAATTAAACAAGCCGTTAACTAAATAAA
AAAAAATAAAGACAGGCCCCTCTCCACATGGAGAGGGGTTTATGTCGTCTTACTTACGTG
CAAAGCTAGCTACAAGACCTTTGATAGCTAGAACGACCATCCAGGCCGTTAACCCTTTTA
TTGGGTGGATATCAACCCGTTCTTCGATATCCAGATTACCCTCATTATTGATTACCTCAA
TTCGTACTTGCATGGTCTACTCCTTGTCAGGATGGGATATGATATTCAAAAGTCTTTTGA
TATAAAGATGGCATATTACCCCACCAATAAAAGGCGGGGTAATATTTATTAAACCATCAG
ATATCCTAATGGAAATAAAGCAAAGAAGCCATATAAAACAACAAGCAGTGTCTTTTGGCG
AACATACCGGGATTTAATACTATCTTTATCTAAGTCAAACTTGACATAAAGATAAATCTG
ATAACTTTTATATCCCATACTAGCCACAAGGGCTATTACTATAAGTTGACACAAAAAGAC
CATAGTGGGATGCTCTTATTATTAGAGAACAATGATCAATTGTTCTTACATAGCATGTCT
ATTTAATATGCATGATTATCATATTAAATAACCACAACCCTAACATAAGGGTAAACATAC
TGCAAATCAAGCCACTTACAATCATTGCTTTTTGTTTGATTGTTTTAGCAGCAGCAAGAA
ATATAATTGAAGTTATTAATCCACTAAATGCAATTAATAGTGGTACTAAAAATAACAATA
CCCGAGCCATATAGACACCTCATCAAAATAGATAAGTTGTACATGTCAGTGAATAGTTAG
TCCAGCAACTAACAATAGTACTTACCCATATGGTAACTAATACAATGCTTACGGAGAGAA
AAAGCATAGCGTACTGGCATGCACTTTTATTAAAGATATCTTTAAACTCAAGATATGTTT
TAATAATAAATATCAGCATGCATGTTAGAAGAATATCAACTAAGATCAAAGTACTGACTA
GAAGCCATGTTGCACCAAAGATCTGATCAACTGTATACATTATTTCCACTCCCATAGTTT
ATAAGCTATAACTACAATTGTAATCATAATCAATTTTTTCTTCATTGTAAATACCCTATA
GTTTTATCACATTTGTGATATACTGTTTAAAATGCTTTAAATACAAAAAATAAAAAAAAG
CATAATCCCTCCCAACTAAGGGAGGGATCATTTATGCCATTTACTATAAACGCATCATGC
GCATACTTTTAGTTTTACCCATGAAATGATTTTCTTTATTTCTAGCGTATCTGCAATGAT
GAGGTATGTTATTACAAACCATGAATGTTAACCTTGGTATTACATCAGGCCAGTTCTTCA
ATGGTCTTAGTCGTCCAAGAATTTGGATGCTGTCCTTTTTAGAGTCAGTTGCTTGTAATA
GAATAACTTCCCTTAAATTCCTAATATCAACACCAGTACCAGATGATTTAATAGTTGATA
CAGTGATATCATTAGTCATTAATCTATCATACGGACTACCCGAAACATGTCTATTAATCT
GAAGATCTGGATATTGTTTCTTAAGATAAGCTGTCAGTACATCGATAAAGTTTACAGTCG
CGCATAATAGCAAACATTTCTGACCTTCAATACGATCCTTTATATAAACGCCATCTACAA
TACGTTTAACCATCTTTAAATAGAAGTCTAGTCGCCTAGGATTCTTCATCATTACTGTTT
CATAACGAGCATGGTTATATGTATTTTTAAATGGAGTTAAATAGTCCTTAGGTTTAATAG
TTGGTTCACTATACAACAAGCCAATAACATTGATGTATGAGTCATATGCAGGTAACCTAC
ACTTTGTAGTAGCAGGTAGCATGACATCAATCATTTTAGTAACAAACTGATTACCAGTGT
ATGGAGTAGCTGATAAATAGATTTGTTTATTGACATTGGTATATAGATCAGTACGAAATA
CTAATCCTGGATCTTCTTGAATCTCATCATTGATCTGCCAGCCTGCTCCAATAACCTCAT
GGAATCTAGGTGGTGGTACATTAAATCCAACTACATCGATTTTTTCACCTAATCGTTCAA
AAGTATCGATATATGCACGGTATGTTGTAGAACTAATGAGAATAATTTCAACACCTTCTA
AATCATTCTCTAGTCCGCGATTAATTAGATTCTGTAACTCAGCAGATCCAGATACTTTTA
AATATTTTATACCTAGCTGATCTTCATAACCAACAAATGTTTCTTTTAATGCTTTCTCCC
AAATACCGAAATATTTAGGTGGAATCATAACAACACCACGTTCTTTCATTACTGCAACTG
AAGCTAAACTAGTTAAGGTTTTACCTTTACCAGTCTGTAAGTCAACACGGGCTGAATGTA
AATGTGGTCTTAGAATATCTTCGCGGATGGTTTCCTGATAATCCCTTAGAACATAATGTT
CAAATAAAGTATAGTCTACTTTAACAGCTGCTGGTACAGGAATATCTACAATTTCAATTC
TAGTAGATGGTATATTCTTATCGGCTAGATAATTAATAAGTTCTTGAAGACATTCACGAT
GGATGAATACAGATTTACCATCTTCTGTTTCACCATAATATTTTTTCTTTATTTCCATTA
TGGTGCGTTGACCAGGTATCTTCTTAGGTTCTTTTAAAACCAGTTTATTAAGATATCCAG
TCATCTTTAAGTATGTTTCCCGATCGTAACCGGATAAACGAACACCATGGGTGTATCGTT
CTGCTGTAAGAATCGGTTTCATAAAAAAATCAACGATCGGTAAGGGGAGCAGTGCTCCCC
AAACCTCACGTCCTCCACGTCTATCGCAGTTTACCACCTTTAACTAGTAAGTCATATGGA
TGGTCATTCCTCATCTTGTTAAGGAAACTGCCTGGGTTATTTAGTGGCTCATGCTGCTTT
TCGAAAGCCATCGCACCACCTAAACTACGACATTGCATTAGCCTATTATATTTTTCAAAT
TGGCCATTAATACCGGGTTTAGGTAAGCGATAGTCTCGATATTGAGCTGAACGGATCATC
ATAGCGTATATCAAGATCTCACAATGGGAAATATTAAGGCTAATCTTTTCATTAGCCATT
GTTGCAAATACAGGTAGTGCTTCAATTGGTGACTTGTAGTTTTTAAGATACGTTTTAGAC
GTATATCCAACTTTCTCAGTGCTTAGTTTACCTGCCTCGGCTGAGTCACTACCGGAATGT
AAGAAACTTTGGAATCGTTTCATTACTTCATACATGTTTACGTGCTTATTTGGTAGCGTT
AAGAATGGTAGGTTAAAATCAAAACCACGAAGACTAATGACGATATTGTCTCGCTGATCT
AGTTCCCAACGGACCATCTTAATATGCTTTAGCATTTCAATAGATAGACTAGCCCTACGG
TTATACAGCGACACAGTCAGAACATCACCACATTCACCATTCACCTCATCATCATAGACT
AGGGCCAGGCTGGTTAGTTCAGTAGCTGAAGTAGCTGGATATGCAGTAAGGTCATCAATC
ATTAAAATGTCAGCAAGGTTTTCTGCCTCTGACCGTGCGATTACTAAACGATAATCCTTC
TGAGTAAGTTCTTTCTTTAGATATAGTGTCTCTGGGATTTCACCTGTTCTTAAATAATTA
CTTTCAATTTTGCCAAGTTTATATTGTTCTACAGCAGAAGACGCATCGGTGTGTTTAGTA
GACAATACTGCTGAAGTAATTTTGTCACCCATCGATACGGCAGCAACCTGACCGATATTA
GTTCCTTTTGGAATATTGATACCTAGTCGGCCATAACAAGTCATACAGATACCTTGACTA
TCTGGATGATTACATCCAAATACAGACCTGAATTTCTGTTTAGTTCCAATTAGGTGTGTT
TCATTTCCACGGATCCAGTCTAGTTTACCATCTTCTTTTTGATAATATTTTCCTTTGAAT
GCTTTTAAAGTTAACTTAGTTACAGGATACTCAGCTAAGATTGTGGTCTTACAATCACCA
GGATGTAGCCTTTGGACATATTGTGCAATAAGCTGAGATTTCCGGTTAAAGTATTCAGTT
ACTCGCAGTAGTTCTTTGTTATATAGTAGTGCTTTAGTACCTGAACGAGATTCAATCATA
TTCTCATACAGATTCCAAATACCATCAATATAACCAGTTGTTACCGGATATTTAAAGATA
TCTGAGTTAATGTCAGTTGGAAAGCCCCGCCAAGCAAATGCTTGTAGAAGCTGTTCAGTT
TTCTGAGTACCAGAACGAAGACCTTCAATGATGCTGTTACCAATGAATTGGGTAGGATCA
TTAAATACTTCTTTGACTTTACCATAACTAATTTTTTCGATACCATAAGTAGTTGGTTCG
ATATTATGGTTAGCCTCACGTACCTTGGGATGATTATATACTTCAGCAATATCAAACATC
GACAAGGTTGCTAGATATTCAGACAGTTTCACAGTGGTCTGATTATACAGCCAGTTCTTA
CCTTCAATAGCCAGTTTAGATAGTACTTCAGGATCTACTGTTTCACCTGACCAATCATAG
ATGTTCCAAATAGCCTTATTTAAATGCTTAACCATGGCCTTAGATGTTACTCGGCCACCT
TCAACATTTAGATAATCTTTAGGGTTATCAGTTAAGATGCGATTGAAATCGATATGGTGA
TCAGATGGAATTGGGCAATCTTTATAATGTTTAAGAGGATACCAACACAGAACGGATAAT
TTGGTAATACGGTCAACTGACACAACCGTTTTACCATCATCAAATTCGATTAGATGCCAT
TCTGAAGGGAGTGACCATAAATCATCATAACTTAGATTTAGAAGATCACGCGCTTTATAA
CGATTAAGATTCATTATTTACCTCTTGTATTATTAAACCCCTGATCAATGATCAGGGGCA
TATTCTAATGCAATACCACTACATTCAAGCAGGTGATCAAACATAGCTACTGGGCGAGAT
CCACCAAATGGAATCTTTTCACGATCAACTGCAACTGGTACAGATGAAGGTTTTTCTGCT
GTTAGCCAAGACTCGATAACCGCAGCATGCGCCAATGGGTTATTAGTTTGGTCTAAGATT
TCAGCAGTCGGACCTGGACCAACTGTACAGTTATATGACCGAGTTTCAGATTCACCGAAT
GATCGAATAGCTGTTTCTCGACCAGGAGTACTAGCCCTATCAGCGTTGTTTAACTTAGAA
GGTAGACCAAATGGTTGTGTTTTTACAGATGCTGCTGCTGACCAGTCTTCACCAATCTTT
TCTAGTAGCATCATGTAAAGTGGACCCATTAAGACATTGTCTTTAGTTGTTACCCACTTA
CCAGCTTGATCTCGATAAGATACCTTTCCATAGTGTGGTCGATACTTATCACTATTAATT
AACTTATTAACTGCTGCCATTAGATCCACTGGATCATCGACTGGTGCGTAGATATATGGA
AACCCATCCATTAGTACTGTTTTAACATGTTCTGCTGGATTTGGGTGTTCCATCATTTTA
CTATGCATTGTTGGAGCAATGATCTCATAGAAACCTAATAGTTCTTTAAATGCATAATCA
ACCCATTGGGTGTTACTCATTACACTATTTAGCTGTTGCTGGGTTGGTTTAGCATGTCGA
TCTAGTCCAGCTTCAACGCGTAGGCGTTGAGCTAGATCTCGTGCAGCTGCGCCAAAACCA
TGTTCATAAATTCGACCATAATTTGAACGACGCATAGTTGAACCACCAAAGATAATTAGA
TCTGCCCTATTACCATTTTCGTCAATAGGCATATCTTCATCTTCCATTACCTTACAGATG
ACGCCCTTCGTTATGTTCAGGTAAGTTCGCTACGCTTACCCCGCCTCTCGTATTGCAGGC
AGCTGCATGTCACCATGCAGACCAGACTATATCTTAACCTTCACCCTAGGTGGTCAGGTC
CCTCCCATTTCCACTCGCTTGAGTGTACTCCGCGTAACCGGATAGTCGTTGAACGTTCAA
TCCATTTCTGGAAAGCTTCGCTGCTGATTGTCTCATTGAGATGTCCCAGCAATTAGAGAG
GATTTACTTGACTCTTTCGAGATCAAGCAGGCAATTTGAAAATATCCATCCATCCAGATT
TTCACCGGATTTTATCCGATGCTTAATAACTGAACGGTCTACATTGAAGTGTTTTGCGCA
ACTAGTTAGCGAGTTAAACCCAATGCACTCTTTATTCTTGAGTGCTTGAACAAATTGTCG
CTGACTACCAGCTGTTGATCTGTGTACTCGATACCGCTCCTCTGTATACGAGTCTCTCAC
CACAAATTCATAAGCTGGGTATTTTTCAGTTGCGTACTTTGCAAGACCAGGAATCGATCT
CCCATAGGGACTATCGTAAAGTTGGTAAGCTTTCAAGAAAGCTGCGTTGGAATAGATTTC
CTCGGCATAGCCATCTGGGTTGATAACTGTCACCATATAACGTGATGGTTTTACTAGTTC
GGTTCGATGCTCATAAAACCAAGGATCCGTTTCATTAGCATATTTAAATTCGAACTTACC
AGCTATCAACTTACCGAACATCTTAGGCTTCAACATTGCAAATGGTGTGTCTTTTGGAAG
ACCCATATGTACACATGCTTGTGCAATTGAATTAAATGCCATTACTTGTTTGGTGAAGAA
GCACCGTGTATAGCAAGCAATTGGATCACGTCTTAATCCATTGTTAACCGCATGGTAGTT
GTTACCCGAATAATCTGTCCATTCAAGATTCTCAAGATCGTTATTAAGTGGATCTCCATC
TTTATGGTTAACCACTGTCAACGTATCAGGTGAATCATTATTTATCCAAGCTAATGCAAT
CGCTCGATGGACAGGTAACGTTTTAGTCAGTGATCCACGAAAGGTACTGACTGCACGATA
ACCATCTAACATAAATGTGGATAAAATCAAATCTCTTTCTATAGACTTAATCACACCGCA
TTTGGTTACCGCATATCCTTCATAACCTGGTATTACTTTAAACTCTTCATTCATCGCATC
TCCCAAGAGATGGATGGATATTAATTACCACCATGAAAATCAGTCATCTTAAAAGCACCA
GCTGGCATCTTCTGAGCTTTATAGGTTACTTCAACTCGCCATTCATCCAGTGGATCTAGT
CGATAGAATCGAGATAGTTTACGGACATTATCAGGTTGAGGTAGGAACATCTGTGCAGTT
ACAATAAGACGTTCAAATTCCTCAGTGATATGTAGATCATCTTTACGACGTGCTAATAGA
CCACGATAAATCTTTAATAGTTCTCGATAATAACTGCTAAGATGAGTATGATATTTAACT
AATTGTGCATCCATCCCTGTTGGAGTGGGTGATGGATTTACACGCTCATCTCGCCAGATA
TCAATATCAATAACCTTCGCACCAGGTGTGCCAATAACAGCTCTATCGAAAGTTCTATCT
AGTGTACGAAGTGCCCTAGGAGTCATCTCGGCAGGAGCTAGATCATCATCATGGTCACGG
ATGGCGAAGATTACGCCATCTGGTCGGATAACATCACCAATGTCTGGGAATGGTTTATAG
ACCTTATCATCGCCATACATGTTTAAGAAGAAAGCTTTCCTACCAGCATTAGCTACAGCC
GTGCTATAACTAGTTGGCATCATCCTTTTAAGGAAATTCTTATTAGCTACGAATCCATCT
TCAATGGTACCAGCGGCAGATAGGAATACAACATTAGCATTAACACCCATCCCTAAAGTA
CCGTCTTTCTTAACTGCACCAGACTGTGCAATCACTGTGTCTTTAGAGAACATTTCATTT
GGGGCAATGGTTTCCCACACTTCTCGATTCTTTACAAGTTCATAACCAAAGTCTTGGTGA
TGACTCATGTACTCGGGAACATGTAAGACACCAATGGTTTTATATTTGTCAAAATAGTTT
TCGTAGATAATAGTTGTTACCGGGTTAGATCTAATACTGTCTTTACCAATACCCGTTGGA
TATTTTCTAAGAACATGTAGTACACGACAATCCTCAGGAAAACGAACATCATGTGTATAC
TTGGCATACTCTAATTCAGCACCCGTTAGGAAATAACGCGATTCATTTCCATTTACTTCT
GGTGCCTGGCCAATATGTGTTACAAACATAGCACCCCGTGCAGATGAGGTAGTGCCATAC
CAAGGATCTAAACCAGTATGCCCTAATAGGGTTAGATCAATTTCACGTTTACCTAGTTCA
GCCATTTTGTTTATCACCATTAATTAGACAAGATGCAGTACTACTATATTTCTTATTTGC
GCTGTTTTTAGATATAGCCTTTTGTAAGACTTCAATGTCTGGTATCACCAATCTCATACC
TCCTGTTTACTAATTACAAATCAATGATATAGATCTTAAATAAAATTTATTGGAATTTAT
TATGGCATTCACACTGTTAGCTAATCAGCCAGATTCAGGTGAATCTGTTTATTATGAACC
AGGGTTCAGATTAATTATTGAGACTCATTTGAATATCTTACGTAATGTCAGATCCACACG
TATTCAGATTCCACCTGAACTGTATTACCAGTTCGAAGGAAACTTTTATGGATACATGGC
AGAACGCCGTGTCAATCCTGATATGTGGTGGATTCAAATGCGTGTTAATGGTTTAGTTAG
CCCTTATGATTTTGGTAAAGTTCTAAGGGATCCTTCAGCTAATGATTACACCAGTTATTT
AATCATACCGAATAACGACATGATTCAACACCTCGTTAGCTTATATCAAACTAAAAAGTT
TTAAAGCAAGCCAGGGGACCCTTAGGCCCCCTGGTTTATGCCGTGTTACAGTAGACGACC
CATCATTTGGTTTCCACCTAGATTCATCATCGCAGTGTTACCATTGCCAGCTAGAGAGGG
GTTTAGTGCTTGTGCAAATCCACCACCCATCGGCATTTGATTGCCCATGAGTTGCATCAT
CTGCGGATGCATATTAGCCATCATTGGTTGCTGTGGCTGCATCATCCCAGGCATTGCAAA
GTTATTGTTCATCATAACAGGTTGTTGCATCATTCCGGGTTGAGCAAATTGGTTTTTCTG
CCCAGCGTTTAGAAGATCTTGGATAGATGCTTTATTGCTAGTTTGAACTTGCTGTAAGGG
ACGTTGAGGTGCCTGAGTCATTGCTACTGCTTGACTCATGGATGGATTAACATTCACAGT
TGTGCCTACTGGTACACGAGTAGAAGTAGTTTGTTCTTTAGCAGTATTACCCACTTTAAC
TACATTATCCTTTTTCTGGACAGGGATTTGGGCAACTGGGTTTACAGTTTCAGAAGCTTC
TTCTTCTACTTCATTGCTGACTCCTTCATTACCATCAAGAGATGGGATCTCACGTTGCAT
CTTTGCAAAATCAGCAATCACCTTCTCATCAACTTTTGAATAAGGTTGAATAGCATCATC
GAAGATCAGACGATACTTATCAATAACACTATTTAGCTGAGTAGTGATTTTGTTAAATGC
CTGGATAAGACATACAAAATAAGGTGCTACTCGCTCAACAGTTCCTGCTGAATATTCGGT
TGGCGAATCACCAAATGGTACAACCAGTTTAAATAGTTCAATTAGAACTGGCCGCTGTTT
CTTAGGTACATTTACTCCAAGAATATCATCCTTAGAATAAAGATCCTCTACGATAGGGAA
CCGAATAACGCAGATACGGTTAACAGTTTTCCCATCATATTTACCATTTGCTTTTAGATA
CACGCTTACAAAACGTGAACGTTTTTCAGCAGCTTTGACAACCTTATTAAACCACTCAAT
GGTATCTGGCTTAGCACCGGATAGTTTCTTTAGGAAGTCTGAACATTCTGGTGGAATGTC
TTTATGCAGAGTAGAGTCGGCAGCTACTTCTAAAATACGTGAAGTAAGATACTTCACAGT
AAACATTAGGTTAGCCTTAACTTGACGCTGCATATGCTGTAGTACTTGAGAAGTACCTAG
CCGAGAGATCTTTTCAGATAGCGGGTGGAAGGCGATGTAATCATCGCCAAGCCCTTTACG
AAGCCAATCTTTAGTAGGTACTACTAGTCGCTTACCATCTACCTTTGCAGGTGCTGGTCG
ACCAAGTGGATCGAGAATACTGATCATATCCTCTTCACCTACTTTGTATCGGAAAGCAGG
TAATACTTGATCACTATAGAACTTGGAAAGAGAAATCGTAGGCATTGTTATCACCAAATT
TAGGATTGATTTGAGCAGGTTGAGCCACAGGGTGCTGATACGGTTGCATGTTAATCGGCT
GACCACTACTGTTTAGAATGTTTTGTTGAGTCTTAGTGGGGATAACATTCTTAACAAGAT
AGGTAAGGTCATTGGAGATCATCGCTGGTGCAGAAGCATCCCGAGTAATAACCGGACTGA
ACAGACTATCAATGAAAGTCGGTGCAATAAATCGTTCTACCGATTCATGATTTAGACTGA
TATCAATAACAGTATCACCAGCTAAGTCAGAGTGCATGGAGATTTTAAACGGAATCTGGT
TACCATAAGAGATACTGTTAATCGCATCGGTAATAAGACGACGTTCAAAGTCATTGATCT
GCGGAGTCATATCAATTCCATCCATGATACTCCTGGTACCATTTGGATGCATTTCGAATA
GATAGTTATTCGCTCCATGTCCATTTGTAATTGCAAAGGAAGTTACCCGGAAATAGTTAT
CCATCATGATTGCTGGAACTACTTGTGCAAGTAGAGATGCTGCAACAGTGTGATGATCTG
CACCACCCCAATGCATGGAATGCTCTGCTCGGTTAACGAAGCGTTGAGAATTACCATTAT
CCATACCAGCTTTAACTACACCATCAACCAGTAATTCAGGGAATACGTGTTGTAGGTCAG
CTAATGTAACAAAGCCCCGCTCCATGTAGTTGCAGCGTTCTTTTAGAATACGCATGAACA
TATTGGAATGGATATCTTGATTTCGAAGTACAGCTGATGCATCACCTAGCATCACTTCAT
CAGAGACACCTCCAAAGATATTATCGTTATCATTATTAGCCATCTCAGCTTCTTTAACAG
AATGCTGCCATGCATTAAGAGTACTAGATAGATAACGAGTAGGAGAAGTATCACGGCGAT
TTGAATATTGGAAAGTGCCTGCTTCACCAACACTGGAGCGTTGGTCAATGATCCGGTCAA
ATACGCCTTGATGTAGTCCAGTTGATTCAATGCGTTTAGCTACTTGCATCGTTTGCTGAA
TTTGGAAAACATCTTCTGGTCGTTGGAGATATGATGAAGTTCCAGTATACATGCTAAACC
CGTTCTGAGGGATAGTAATATCACTCATGATATCGACGGGTGCAAGGATCTGGTTAGAAC
CTAGGATATGTGCTTGTTTGATTACCCCAAACTCAGTTGGTCGTTGACGCTCTACAATAA
CAGTTTCACTATTAAAATAGATACGCATGTTAGGATCTAGACTATTGCTAAGCGGACTAA
TAGAGCAATGGTCAGAGCAACCAAATAGAATCCGGCAAGTACGGGTATTACCAAAGAAAC
GGTTATTCTCCCATACTTTCATCATGAACCTAAACCGGCGTTCACCCCAACCATTGACGA
TAGGGACTTCGCCTTGAGTTTGAGCACGTGGTGCGATCACATCAGCTGCAATTTGCTGTA
CAGCTGCAACACCTAGATTACGACCATCATTAGTTGCTTCTTTTAAACGTTCAATAGAGT
TCTCAGTGACCTCTACAGTAAATGGTCGAACATGTTCAGGTTGATAAGTACCAGTTTGTA
CTAGAATGAATGATTCAATCTGTACTTCAGTATTACCGCCCATTGCTGAATTATTAAAAC
CTAACATAGAACTACCTCAATTATTACTGATTAAGTTTACCCAGATGAATTACCAGTTCG
GTGATCACCGACTTAATTGTGGCCGGTACAACAAGGACTTTATTCTGAGTTACCTGTTCT
GCTTCTTTGAATAAACGATCGGGTCCATGATAGATCCAGTTACTAGATCTAATGGATGCA
TGTGCAGATTGCACAGCAATGCCCGCAATATTTACAGGTGCTACTGGAACACCATTAATA
GCTCGCTGTTGTCGTTGATGTGGATAAAGCTCATCCATTACGTCCTTATATTTAATTTGA
ATACGTGTACTATTACGTGGTTGAATGGACATTGCATGATCACCACTATAATACAATTCT
ACTTGCATAAATACAGCAACTTGTTGGAAACCCCAATGCCACATTAGTGACTGAGCTGCT
GCCAACAAATGGTTAACTGCATTCTTATTAATGTGGCTGAATGCTCTTGCAGGAAAAGCT
TTGTGCATTACCCATTGCGCAAGTAATATCTGGTGTGGATAGATATCTTTAGTAGCTGTA
ACTGCAACGCAATCTAGACATTCCTGAATTAACTCTACTGGGACAGTGTCATCGATTTTG
TGAACCAGTTTAACTACATCAAGAGCATCTAAGTTATAAGCTACAATATCACCAGGAGTA
ACTCGTTGTTTAGTTTTATGCGATTCGATAAATGATGTTTTATCATCATCATCAATACCA
CCTTTATCTGGCCGTTTATCATTAACCCGGTCAGCAGTTGATCGTTCAGCAGGGTTTAGA
TTTGACTTAACATATCTAAACATATTTGAAACGATGGAATGTGAGGTGGCATCGTTTAAT
GGTAGGATGGTTAATCGCCTAACCATAACTTTAGCCTGTAAGATATCCGGCACTTCAGCT
GAACTCATACCTCTATAAAGGTTGGCTAAGGTGGGTGCTTCATCTTCTACACAAAACTTA
ACATATGCACTTAGCTTATCATAAGCTGTTTCTACTGGAGTTCCATCAATTGATATTTCA
TCAACAGGCCAATGGTTAACCTCACAATCCGAGATGAGACTAATTACTTCACACTCTTTA
TGCATTTCCTGAGAAGTGCCCTGATCAATAAACTCGCCCCAAATAGGTACTAAAGGACGA
AGAGCTAGCGATACAGATGCAAGGTTAATATAGTCATGTTTCAGATATGTTTGATCTGGT
CGATTATATCGTGAATCATCAGTGATAACTTCTTCAATATCTACTGGAATATAAAGTTTA
CCAATAGTTAGTAGCCATCGCCGAAAACTATTCATCGGCATGGCTTTATACATCTCTTTG
ATGTAATAGGATAGACTCATTGCAATCCTCATCGGATCCATAGCCATATCCATGATCTTA
CGAATTTCTACATAACAATTCCAGATATGTTGTTGTGCCTCAGCACTTAGGCCACTCCAA
TAATCATTGATTTCTTGGAATACTCGTTGTTGATCTTGTACACGGATTTTACTATAAGCC
GTAGTTGTCCAAGTTAAACGTTTACCATTGTGTTCTGTAGCCACTTCAGTAAAACCAGTA
CCTTGAACTCCTGTAACGATGATTTGCATTTTAACCTCTTTTACCAATGTAGATACATGT
TAGTAATATAGATCTCAAATTTTTTTTAGTATAACGGCATTTAGTACTGAGGGCATATCG
GTGCCTACTTCGAGAGTAAGCACCGAAATATGTTTTAGAAGGAAGGCATATCCTCATCAA
AAGCTGAGTCATTATAACTATTATTAGCCGGCTTAGCATAACTATTATTGCCGCCGTTGT
TATAGTTATTGCGCTGACCACCGTTATTGTAACCGACGCTATTACCATTACCCTGATTGA
AATTAGCAGGGTTAGATACACCTTTAGCATCTGGATCGAATTCAGTTACTAGAAGATGCA
TTACTAGTTTATATGCGGGGTCTAGGAAACCGTATGCATAAGCATCGGACATTTCCTTAG
GTGATAGTTGACTACCATCTGCTTTGCGGATATCATGGAATTGGGATGGGCCAAAGAAGA
ATTTGATCTTTGGACGATCAGGTTGAGTGCTTAGCACTGCGATGTAAATTCGACCATCTT
GTGCACGGCCAACATGTAGCTTAGAAAGCTCAATAAAATTATCACGCTTCTTACCAGCTA
CGAAGTTATTGCGGTAAACAAAGACACGTTCAGTATCTTCAGTTTTACCTTCAATTAAGT
CCATTGCAAACTTAATGAAACATGCAAAGGTTGCTAGGTCAGTATTGAACTCAATTTTAC
CATGGTCTTTATCACCAGGTACATTAGTACGAACAGTAACCCGTGGTTGGTTTTTAAACA
GACCAAAACGCAGCGTCGGTCGTTTTTGAGAACCCTCACATGGTTTGGCATGTAGCCAAG
TATTCATTACATTAAAAGGGTTAACTACGGGTGCATTAGACTGCTGTTGTGACATGATGT
GATAACTCCAAATTAAAACTAGATTCTTTTTAACTAGATGGGAAATGATTTCTACGTAAT
ATTATCGCAACAAGTATTTCTTCTATTTGAATTTCTTGTATGCTTCTTTTAACTCTTTAG
AACCATAAGCAGTAATGTCATGATGAACTTTATCTGGTGTTGTTAATCCTGTCCATTTTC
TAGATTGTGCCAATTGTTTTAACTGTGCACGAATAGCTTTGTCTTGTGGTTCAAGTAATT
GTTTATCGCCAAATATCTGTATAGCAAATGATGTAAAAGGAAATGGAGTATCTTCTTTTA
AACCATTTAGTTTAGTATACCACATGTTGTATGTCTTTATTTTGCCCGTATGGGATTCCA
ATAAAAGAAGTCTATTAAAATGGGGTTTCCATAATAATTCATGTGGGAAATGGGTTAATA
GTAAAACAGTACCTTCAACCTTTGGAGGTTTATTAGATATTTCCATTGTAGTTAAATCTG
TATCTTTTAACTTCTGGTATAGTTCAATTGACACAAATCGTTCATAAACATCATAAGCTA
ATTGTTGCGGTGTTCGTGATACTTTAAATGTAGCTTTAGGGAATAACCACTTCACTGCAT
CTCGACCAGTAATGTAATATTTTACAGTTGCTTTTATCCCCGATTGCCGTAAAGCAACCG
GGATTGTTTGTGCTTCTTGAAATAAAACATCGACTGCTGCAACCAAGTTAATATTAGTTT
GTTGTTCAGACTTCATAGCCTGCCACATATTTCTTGCAAGAGTACGGACATTAATCCATA
TTTCTTTAATAGACTTAACATCAGTTGGTTGTCTAGGCTTTTGTGGATGTGTTCCTATAA
GCCCTTCGAGAGCAAAAGAAGTTCCAATGCTAATCGGAATATTAGCACCTACATCTCTCT
CGATAATATCCATTATTATTGCCTTTTATTTGAACTCTGTCAATAATCCCAATACCCGCT
GGATCTGAGCAGCGTCCGCTGTAAATTTTTCAATATCTGGAATAATTAAATCAGAAATAC
TTTCTGGAGTTATATCCAAAAATTCTGATAAATCAAAATTCTTTAGGCTATCTGACACAG
TATTTTTCTTTGTAGTAACTTTTTCAATTAAGATACTCCATGAAAAATTAGGATATTCAT
TTTGAAATGTATCGAGATCACCAGTAGCTACATCACCTGGATTACATCTTAACCTAATTG
CAGAGTTAGTTGGTAGTGTCTTGATATGTTCTTTAACAGTGTAATTAAGTTGCTTAGTAT
CCATTCCATGGACATTCAAAGTATCATATCTACGCGCACCCTTATTCTCAAGGAATGTAG
CAGTCCATTCGCCATTTTCTTTCACCACAACATCAAATAATCCTTTTGGAATTTCATCGT
TGTGACTTAATCGGTCAAATCCACCGGCAGCTAAAATGCGTTCATAGATAGACATCTCAT
GAACATGTCCACCATTGATCATATATTTAGTTAATGCTAAATATTCATCTGGATCATGTG
TTGGCTCTTCAACAATGCTAGGTAGCTGGTGTTTAAATGCACCATGTAAAATAGTGAAGT
CAACTTGAGATATACCTTTCTTATGCATTAAATCTTTTACTTGATTTAAAGTCTCTGATG
TGGATGGATTCCATTTATCAGGGACATAAAGGAAATATGCATCTAGTTTCTCAATATACT
CGATATCGAGAATCTTTGAATAATGCAAATCAACTTTAATATTAGCATTATTCTTTTGTT
CTACGAAAAACTGCATTTGCCCACGATCGTGTGATGGAGTCCCTTCAACAATACGGATCA
TTACATTATACATTTCACATTTGTAGAGAAGTATTGTAATAAATCTATCAATTGCGTATG
CAGCTGGATCACCATTGTTTAATTGGCGATCAAATAAATCCCCTGTAATAATCAACATAT
CAATATCTTTTAAAATTGTATCAGGGACAGTTTTCCAAAAGTTATTGATGATTAATTCAG
TAGGAGTTTGGCGATGCCCCATATGGACATCGCCTACTGTTAAATATCGAAATATGCCAG
GTTTCTTTGTTTTATCAGTCTTCATATTCGTCACCGTAAACACATTCATCATTCTCAGGT
GTATCTACTTGCACACCTCCAGATTTAGATGAAGATGCATTCGGTAACTCATATAACGGA
GTACCATCAGTATTTTTTAATACCCGACCATATGCTGCTAAGATTTGGATTAACACTTGT
AGAAGTTGTTTTTCCAATGGTTCATTATAAACACACCGCTCTAAGAATTGAGCTGCTTCT
TTAGCAAACCCACCGCGTGGATTGCGATTATGTTCTTTATTATAATAATCCATTACATTA
GAAATACTAGCAACATTCTCACCAGATGAGATTGCAACACTTGGACGAGGATAAAGTGCT
GGAACTGTAAAAAGTACAACTGCTTCTTTGGTATTAACACTTGCTACACGTAATGGATTT
GATAGCTCATGAACATATTTACGATATTCATCTAATGCACCAGGTGCCCACGGACATTCA
AGTAACGGTAATATATTCTTTACAAAAACGTCCTCATGGACAATAGGTATCTCTTTCTGA
TGAATGCTAACCATGGCTTGCATTGCATCCATGATCATGTTATCGCTATCTGTAACTCTA
GTGTTCGGTTCCGGAGTTGCCATCTTCTTTACCCATAACTTGTTCAATCTGTTGTTTAGT
CAGAGTAGTGATGATATTGAAACGAGCTTCTTCTTTGTTTTCCATTAGTTGAGGATTACT
ATTTAGGTAATCAATCAACGCATCGCAGTTTTCAGTAGTGAACTTATTAGGTGTTCCAAC
AGTAGCAAACATATAACCTTTATCACCGCCTTTACGAACAATGACCGAGTCCATATGTTC
TTTCTGATTAACTCCATCTACTTCAACTAGTGAGAATACTGGTTTAGTATCACCATTGAT
AGCTCGCATTGCGTCATCAACTTCACGCCGAATACTTTCACCCATATTGCAAAGAATATC
TGCAAGAACTCGACTAACGCGAATATTAAATGTTGCAGTTTCTTCAGCTTTAACTTTTAC
CAATTGTTGGAAAGACTCATCTATAACTTTAGCATGTTTCGTTAGATTCCTAACTGTTAC
TTCTAGCATATGAATTCGTTTTAATAGCATGGAGTTCATAGCTTCGCTTTCATTTTGTTC
AACTTCACTAGTCATTTATTCACCATTATTAAGTCTTGCAATTTCAAGAATATTAGAATA
TTGATAACGTACGAGCTTACCGATCTGAACAGGTTTACCTTGAACTAATATAGTAGCAGT
AAATGTGATGGACATTTTACCATCACCTGTATTTGATTCATCTTTTACTATTACATTAGA
ATCAATTAGTTCATTACCAAATACGGTATTAAACTTATCGCTTAAGATACGTCGTAGTTC
TATCTCTAATTGTGGATAATCATTGGCATATACCTTCAACAGATAAGGTAATGAAGTACT
TCTTTCCCTATTAATAATATCTTGGGATGGATTAGATAGAATGAAGTATGAAATCGTTTT
ATCTGCTACTTGTTCAGGTGTATCAATCCAACCACTAACAGATACTGTTGGAATCGTTTT
TGGCATATTGAACTCCAAAACTAAAGAAAAAAATTAAGTTAAGATGGGAGCCAAATGGCT
CCCTTTATATTACTTGATACATACAGTTAAATACTTACATTAACTAGTATACTAATATTT
ACATGCAATTAGTTCAATGTGATACACAATAACAGCATCTTCTTTTTTCTTGAGTGTACT
TGTTAAAACGATATCGTGTTCAGTAACACATGCATGTACACTAATGGGTCTGGTAGTAAT
TTCACTACTTGTCCCTGAAAAATGGGTTGTAGCTACATATAGCTGGGTATCATCAATTAA
CCCATTCGGATCTCTACCAACATGTACAAATGGTGGTCTGTTAGCCACCTTAGGATAGTC
GTCTTTAATAACAAGATGGCCATCTTCAGATGGGTAAGCATAGTCATATCCAGTCACATA
GATACGTAGAGTACCATTTTTAAATGCTTCTTTAAGATAAGAATATTCTTCAAGATTAAA
ACGAGTTGAATCAACTACAGCAGTAGTTAATTTAAGAATAGGGTGCAGTGGCATTGATGG
GTTATTGTGCTTAATTAATAGCATCGGTATTTTCCTTAACGTAATAAGCAATATGTGGAT
GGTTCCACAATGGACTCTCATCACAAAAACTATAACTATGTATTTCCTCGCCAGTATCTA
ATTTAACAGAGACCACTTCACCTTCAATAACTGTAAAAATAAATTTAGTATCAGGTGAAA
CATAATCACCAGTTACATAGTCATTATCAGGAAGATACAATAGTCGTGTTTGATTATCGT
GTTTAAGTTCTATTCCATCTGTACTATTAAAATACATATATCCCGTACCATCTGGACTTT
TTTGAAACATCTTTAGTTTATTTGGTACGAGTAGATCAATTGACATAGATCTCTCCTAAA
CGATAGAAGAGAGCCCCTGTACGGGACTCTCATTTATTTAACCTAGAAGATTACCTACGA
CTGAAGTTGGATCTTCTTCACCTGCGTCTAGATGCCCATCAATAGCATTCCACATACGGA
TGATGTCAACTTTTTCATGCAAACTAAGTTTACGATCACTCTTGATATCTTCGTGATAGT
TTTTATAACCGAATTCTTTCTTATCGGTATACATAACTCCATCCATCACGCGACGATAAT
CAAACTGTCGCTCACCTACTGCATCACCTTGGATGTTATTATAAGAACCACTATATCCAT
CAACTTCATTATTAAGATACATAGTTCTTAGACGTTCATGCGCCATAACCCAACGTTGCA
TAATCGGACCAGCTGTTTGTAGTTCAGCTAATGTCTCAAGATACACAATGTTGTTCGACT
GCCATAAATTATCTTTCTTTGTGGTTAGATTCCGCAGTGCTTGAACAGCAGCTGATTCAC
TAATCATACTGTACATATTTTGTGCAGTATTGAAAAAGCTAGCAGCAGCAGTTGACATAG
TTGAACGCAGTGCCTGACTACGCTCACCCAACCATTGGGTTGTTTGCTGTGTAAGATGGC
CACCTGCACAGATGTCCAAGAGGTCTACACCATGTGCATCTACAATCATTATTCACCTCA
GATACTGTACTCATATACCCAGCACAGATTAAATAGACGTTGCATAATAGGTTACCTCTT
AAAGCTTAGAGATCATTTCGACATATGCATCGACAAACCTTCTGTTTACCACTACATCAC
CATTCCCACGAGTGATATATCTCCCAGTTAATATAGCTACCGATCTGGCTTCACGTTCAT
CTCGTTCGGCTTGTGATAAACGTTCCGGGTTACGTCGAATAATTTCACGAATATTAGACA
TTGGGTATTCCCCTTATCACTATTGCACCTTAGTGATATAAATCTGAAATTGAATTCAGT
GTAACTTCACGGCATAGTTGTCCAAGAGGTCTACACCATGGGCGTCTATAATCATTATAC
ACTTCAGTTATTGGCCGTATATGACCAGCATAGATTAAACGGATGTTGCATAGGTAAATT
AAATGTGTCTAGTTCCTGACCGCACCAATTTACAGCTTTTGTAACACCTTCAACAGTTAC
AGTTAATTCAACATAGATGTCATGTTCAGGTGCAGTTACCACAATAGCGATTGGATCATC
ATGTTTAATACGGAATCCAAACTTAGGATGATGGAATCCCGTATACTCACCTTTAGTATT
TTCTAGAATACATTCAAATAGATTAGTTTCTTTATTAAGTTTAAATGTAACTAGTTTATC
TGAATCTTTAAGTAAACCATTATCCCCAAATACATTGAACCGATAATGCTTATGGCAGAT
ACCAAGTGTTTTTGGTACATCATGGGAATTATCGATTTGCTTAGTACATTCGTTGCATTT
ACTATCCATCTTTATTTACCTTTAATAGATTTGTTTATCAAAAGAACAATTGATTGGAAT
AGTGATAACTTCATTAGTAATTACATGTTTGCCTGTCTTTTTAAAGAATTGGGCAAATTT
TGAAGTAGCTGGAATTACAAAGGTATCATCCCGATCATTATTTGACATATTGTACAGAGT
AAATTTACCGTCTAAATCAAACCTAATTTGCCATTCAATATCACGTACTTTGAAATCTAT
ACCAAATCTTAAGTGATCATCAATCAATTCAGAACCACCGTATTCATAAGCTCCGGTGAA
ATTCCAATTAATTTCTGCGAATTGGAAAGTTACAGGATGTTCGCCATATTCATCTTGTAT
ATAGTAACCATCATGAGCTTTCCAATAGATAACTAAATCATGCATGCTACCAGCCTCAAA
GAAATAATAGGGGAGTTAGACTCCCCTATTAATTTATTTTGCTTTTAGCCACTCTTCTAA
TGGCGGTAAATACTTCTCATGTGCCCAGTTAATAATTGTTTCTACAACAGGACCCTGTAA
TTCAAGGTTACCTGAGATTTCATGTGGTTCATCAATAGATAGAACCCATGTATGTGGCGC
GATGCGTTCAGTTGCTTTGGTTAAATACACATCGGGCATTAATGTTAAATTAAGTTGCAC
TATCTTCATACAGCATCGTCAGCGACATGTCTAAAAATCATCTGGGTCATTTTCATATTT
AAAAATGAACCCATTAATTGGTCGACGGTCTTTCTTTTTGAGTTTGCTAGTTACTACTTT
CTTTGATAATCCAAGATCGAGATATGCCTGCTTGGCTGATTTGTATTTCAAAATCTCGCC
AGTTTCCACATTTTTCACAAGTGTGGTCTTATTTTTTAGCCTGACTATACTCAATGGATT
CATCCCAGGTTGGCCATTCTGAACCAACCCGACGTACAATGTACCTATCCATAATGACTG
GATAAATTGCATGTCCGCTAACTTTCAAAGAAATCGTACCTACACTAACCCCTATCGCCT
CACTTGCTTTCTTTTGCGAGTCATATTTCCTGATTTCACCCGTAACGATATCCTTTATCT
CAATACCGTCGTGCTCAAGAACAATTCCGTCCGTGTTAATTCCGCGATTTTTCAACATTC
TCATGATCGCGGTATTCTTTGCTGATCCAATATAACCGGTTTTGATTGCATTTCCATGCA
ACACATTTTCACTCTGGGTTACCCATTCTAGGTTATCGGGTACTGTATTGTCACGATCAC
CATCTATGTGGTTGACCACTTTCTCATTGATGGTGTTATCGTATTCAATAAATGTCAGTG
CAGCTACACGGTGTAAATTCGTTCCTACATAGGTGCCATCATCTCGACGCAATCCAAGAT
CTCTGTATCCAGTTGTCGCCAAACTTGTTAATTTAGCTGAACCTTCAAAAAGACTTATGA
CCTTGAGTTCATCATTGATTGCATAGCAGGTAAAACTAGGGATGAAATAATATCCTGGGA
AGGTGGGAACCTCCAATCCTTCTTCCGGGAATTTCCAAATCAAGTTTCTAGGATGAATAT
TGTCTTCATCTCCATCAGAAAACAATATTTCGCAGCGGCTAACAAACTCTAATGGCGCAT
GCCAGCCTTTAAAACACACTGCTATCAACTTAGTAACATGAATGGGTAGTCTATGCCATG
GTAATTCAAGAACGTAATGTCGGTTTATTGGATCTATCAATTTATCCAAAATCGATCATA
GACTAACCCATCCAGAGTCAGTCTATAACGCCCGAATGTACCGGGAATCACAATTTCTCT
CGATTTGGGTATACGACTGTAAATAAACACATATCCTCCATTGGAAGACATGTCGCTGCT
GTACCGTCCACTTAATATCTCCCGACATTAAGTAACTGCTCTAGCTTTCACTAGATGGTG
AGACTATATCTTCTTCCCAGAGGGAAGCCTTCTATTTCGGACCACTTGGCCCTACTCTAC
TCCCTTGGCTGCTTTCGCAGTGGTTTCGATAGTCGTTGAACGTTCTTCCTATCTCGCCTC
ACGGCAATACGTAGAAGCTTCGCTGCTGATTGTCCAATCCTAACACTTTTCAAACCGTTA
CATTTTAGCTTTCGCTATCTGCTTTGGTAGTTAGGCTCTAAGGAGGTTCCAGCAATTAAG
AAGGTTAGGAGACCAATTAAAATCTCCGTCGAAATCTGCGTTTGGAGCCTTAAGGCATAA
AACTGACATGCTGATAGAATTATCATTAATGTCATCTTTTACTTTAGTAATAAAGAACTG
CTGCGTAGATCCACGTTGAAGCGTTGGCAATATCTTCAGTTAGTTCGTTAGGCTAACCCG
CACCATTACGTGCAGCTCTAGCTCTCGCTAGACGTTGAGACTATATCTTCACCTTTGGCT
TTACCCAGTAAGGTGCTTCCCACTTCGAGTCCACTTGGACCCTACGTGCCAATGCACTAG
TCGTTGAACGTTCCACATATTACAGTTAAGTAACTTAGCGGCTTAGCTGCTGATTGACTC
TATTCAGTTATTCTTAAGCATTCATAGAACCTTTCGGAACTATTGTAGCTAACTGACCTA
ACGAGTTGTTCCAGCAATTCAAGAAGTTTTGTCGAGTATCATTTCTGATACAAGGAAACC
CATATTAAGTTAAGTCGCTACACTAATTGCTTAGGTGGTATGTAGCTTCTCTCTACGCGC
TTTGTATCTATTGTAATCATCAATGGATTTGTTAACCATTTCTTGGGTTAGGTTAGAAAC
GTATTCGAATAGCCGTTCCTTATCATCGACCCTACAGATAACAACACCGTTGAATAATCT
TAGATCTGGTTTCTTTAAAATGTCCTGCACTGACCCTTTAATTAACCCGGTTCTAAGTTG
TAACTGCGTTAGGTTTACATATCGTCCAAAGGTGTTGGTCTTGAAGTCTATGCAATAAAC
TTCAATTGCATTCTTACGCTTCTGTGGGTGATAATCCCCACTTACTTCGAATACATATTT
ACCTCTGAATGGTTTAACTAAATGACCCAATACGACATGTAACCCTTTATTGTGACCAAG
CTCTAGGAACTCGGCTACTTCTTTCATCGAGATGAAGTTATTTACTTCACCAGTCAGAAT
GTTTGTACATTTAACAGCGAGTGCATGTTTATTGGCACCGGTTATAAATGCATGCTTAAC
ATTACCGCTACGAGTTTCCCACTCTAGGTTAAGGTAATGATTATTATGCTTAATGGTATC
TTTGTGGTTAACGTCCAACTCATTAAAGTTTTCAGGTAACCCATGAAACGCCATGCACAC
AAAACGATGAACATAACGAATCTTTCTAATACCGTCATCATTGTGGATGTTAACGGTAAG
GTAAGTACCTTTAGTTGTCTTATTCTCGTGTTGTGAGATTAATTCGCCTTTAAGGTTCCT
AACTTGACCATGATCACTGACTTCGTAATTATTGAAACCAGGTATTTTACGCCATCTTAT
TTCTTCATTCATAATTCCTCATTAGAGATTCCTGGTTACCACACCCAGAATCTGTAGCGC
TTTAACTTAATAGATTAATTTCGGTGAAAAGTACAACCCATCCCTTTATAAGGGGCTGCT
TCTGCAATTAACTCTTTAAATAGATCTGCGATGATTTGGTTATATTGAAGTACATTCTCA
TATACAAATGAGAATGCTTCACGAGTTGTCATGTTAAACTTAGCTTTTAACTTGTTAGTT
AGATGATACTTTAATAATTGACAACCTACTCCCCAAGGGATATGTAATTCATCATAATCA
TGCGGGTCACTAATAGAAGTGATTACTGCACGTGCCGTAGCGTTGAGACGAGCACCAAAC
ATGTGACGCCGAGCAAGACCAGGCTTTTGAGCAATTCGTGATTTTGCGTAGATCTCGTAG
AACTGGCCATATAGTCTTAGTCCTCGCATAGTCCTATTCTGAGCTTTAATTGGACTAAGT
GGTACAGATGATGCATCAATACTAGCGAAGGTTAAGGTAGCATCAATTGCTGCTTCAATT
GGTTTATCTAAGTAAGTACCAGATGTGGTTGATTCTGCTACGAAACAGAGTTTAGAAGGA
ACTGGTAAATATTTAGGGAATAACTTATCTTTATTCTGAGCAACGAATTGAGCGAATTCG
GATTTATTATTAGAGATAATATTTGCATCTAGTAGGAATTGGAAGATCTCATTGAAATTA
TCAATGAAGTGATTTAATCCTCTTTCAAATCCACGATGCAGTAGCCTATCTACTTTTCTG
CGTGTTTCTTTAGAACCAATAGATTCCACATCGTATCGATAAGAAGTATCAGTTAGATAT
GCTAAGAAATCGAATTCTTTAGTTACTAGATATCCGGTTAGCATAATGATTAAACGTGGG
TTAATTAGACTACGTACATGTTTTGGTGTACGAACCCACATTGATGGTTCGATAGGACGG
CTAGAAGTATTGACAACTGGAGTATTACAAATATCACAGATTACTCCGAGTTTATGTGCG
TCTTCGATTGCTCGACAATCACAGCTTACACTACTTTCAATTGCTTCTGAATCTTGGAAA
TGAGAATAGAAATGCCTATCAAATTCTTCTTTCTCATCTGAGTTACTCGTATTGTAATCA
TTGGCATAAATTCGTTTACCCGTAAATTGATCATGAACTTCATTATGATCAACAACTTTG
GCATAAAGACCCATACCAAACTCCTATTTATCGATTAACAAAAAAAGGAGATATAAGCGG
CCCCCGAAGGAGCCGCTATATCAGTTTTTACAATGGACCACCAACGGAGCCTGGCCCGAT
CCTAGCGTCATCTACAAAGCTTAACCTCCGCTCTTCTCTGTAGCATACCGGCACTAGTAT
CATTTACCATCCATTGTAGTTTTTGATCACACGTCACCTTTTGAGTAACTCCTTTCGGTT
AAGTGATCTATCATCCTCGTGTAGTGATAGGTCGACCTAGACTAAGTCCACTAGTCGATT
ACTTCACGGAACCTACTCCTAACCGTTATCACCGAAGTGAACCAGGGTCGCAGTATTAGA
ATCGGGATGTTACTTATTTAACATCCCTATCTACCGAGGTATTTCCTATTAGTACCAGGT
ACCCGGTGCATTATAGGAAGGAGCGCCGTAAGTTTGACCCATGCTGCCAGCCATACCAAC
TTGTGCACTACCAGAAACAGCCATGTTACCCATACCGGTGTAACCAGCGAAGCGCTGTTG
ACCGAAGTTGGTAATTAGGTTTTCCATGGTTACAGTTACACCAGCGGCTGCTGCTGCGGC
ATCCATTGCGGTGATGAATTTCGGGTTAAAGGTTAGACGACGAGCACGACCAGTATAGGT
AACGTTACCTAGGTACATCTTGTCGAAGCCTTTACTGTTATTCATACGACGCACAATGTG
GTCATTACCTACCTGAGTAGCATACCAGGTCTTCCACTCTTGGTCATTACCTTCAGACAT
GTTCATGGCACCTAGAACGTCAAGATCACGACGGTCGCTTAGTTCGCCATGCTCATCAGT
GTAGTGACCGAGGTCTACTTCATGATGGTTGTCAAAGATAATCGGACCAGCTTCAGCTAG
GTTATAGAACTGACGGAAATCAGTGCCGTATAGGTTAGTTAGAGCTTGGAAGATCAGGCT
AACTGCACGAGCTTGGTTTACGCCACCAGCTGCATCTAGGATGACTTGCTCAATAGCTGA
GTTGTCGCCCATTGGATCGATATCGATCTGGAATGCAGGGTTCTGGTTAACCATCTTGTT
CATTAGCATTACGAAGTTGCTGTCGTCTGCCATGAATGCTTCGGTGCGAGTTTCAATCGC
TTTACCTAGCTCTGAGTAATAACCAAGTGCACCAATATCACGCATCTTGCTAGTAGCGAT
CTTCGGCATTAGGGTACGAGCCCATGCCTGGGATGCAGTTGCACGATACGCATTACCGAG
TGCGAACAGCCACATTTCTAGAGTCCATGCTTGGATCCAGGATGCCTTACGAACGTCAGT
GATAACGATGTAAGGAGTGAACTGCGGAGGTAGAGCTGCACCAGGTAGAGTTACGCCAAA
CATACCTTGTTGCTGGCCAGTGGTCGGAGTCCAATGTAGATCTACGAATAGACTAACTTG
GTTTAGCTGGCTATCGGTATCATAGAACTCGTTCTCAGGTACGTTGGTTTTCTTACCACG
GCTAGTGGAGATGACCATGTCAGAACGGATCGGATTACCACAGCTATCTTTAACCGGAAT
ACCATTGTAGTCTAGGTTGCAGGTAAGCTGTTCGTTTTCTGCTTTGATGTGGGTCGCAAT
AGAGAACGGAGTCTCATTGTTACGACGTGCAATAGCATCTTCACAGATGTTAACAGATTC
AACTAGTAGGTTCTTAACAGCTAGTTCATCTTTGAAGTCGAAGTCAGCATACACTGCACG
GGGACCGGCAGATAGTACTTCTAGACCAGGGATGTTGTAACGGTTCCGTAGGAACTCGCC
GATCTTGCTCCAGTATTGAGCAGTGAATACATCACGCGGTAGTACCTTCTCTTCAATGAT
GTCGTCGGTTAGACCATTGTTGATTTTGTGGGTACGGGGACGGATGCGAGTACCAGGTAG
GTTATCGAGTACTAGAGTACGAACGAATGCCTTCATGGAACCATTGATATCTTTAGCTAG
TTTAACAATGAGTAGACCAGCCATACCAACTTGTTGCGCATCACGATCGAAACGATGGAT
ATCGAAGTCATCAGGTAGGTCCTGATGGCGGATTGCTTCTTCTTTTACTTTAGTGAATAC
TTGTAGGGCATCTGCTGAACGAGCATCAGAGCCATCTAGTCGACCAGAGCGACGCATTAG
ATGGTTAATGCCAGTAGTAGACCCAGGCGCGCCAGTAGGACGTTGAGCTTGACGCTGTGC
AGTGGGTTGCGGAGCAGGAGCAGTAGCTTGAGTTTGAACGGTGCCGATTTCGTTTTCGTT
AACAGCCATTTTAAATACCTTTACGATTATGGTTTCTTGATCAAGAAGAACCTAAGTACT
TAGTATACTAGATTCAATTTAGTAATATAAATCTCAAATTTTTTTCATTACAACATAGAC
GCTATAATCTGCTATCAGCAGATCGTAACGAACACTATGTTCACTACATATTATTACAAT
CTGAGTATTATTTATTTTTCATTTTACAATTTCCATATCTTCTTTTTTGAGTTTATGGGT
ATAAAGTTTGAATCCAGAAACTAATTTACGTCAGTCCTGTATTAAATAATAACTATCAGT
ATTTTTATAAAATGCAGTTATCATTTACTTTGCATATATTTACGAACTGTTCCAAAAAGA
GAATGGTACAGTTTGTATAGTTTACTAACTGGATCAACTTTATTATTATCAGTTGTGTCA
AATTTAGCAACAAATGGTGAATGTGCTGAAGCAGATGTTTCATTTACTTTAACTACACTA
CCAAATCGATGCGCGACTAATAGCTTAACTAATTCCTCATGGAGTTTAGTAGCAGTTGCT
AATGGATTGGGGTCATTAGGATCTATCTTAACTCTACGCTCTACATTGAAAAAGTTAACT
ACAATTTCTTTGCTAGTCATGTATACAGTAAGATCTTTGTTATTACCTAAATATAGTGGC
GGGTCTTCAACATTATTTTTAGCCATTGTATATTCCTATTTGTAATTACTACTGTTGTTA
CCAAACTATGATATAGATCTCAATTAATTTTTAATCGGATAATAACCATGTATAATTTAT
TTAAGAATGCTCCGTCACGTAAATTGGGGCAGGTGGTTGATCCTAACATCCATTATATCC
GACGAATCTACGCTGAGCAAATTAGGGACGTTAAAAGTTATTATAGACGGGCACCGAAGT
ATGTTGAATCTAAAAATATATTAGCACAAATGATTCGACATTTTAACGTAGAGTTACTAA
GTGATGATGCTACTTTTATAAAGAACGTGGACGATCGTTCACGTGCTATTATTCGTTCAT
TTGGTATTACATCATCTTTAAATAAAGGTAAGGTTCATGTAGGTGGTGTTACACTTGGCC
CTCAGACTGAAGAAGTTTTAGTATCCACATCAGAGAGCTTTGATCTAAAAGATCTAAATA
AAACATGGTATAAACTTTCCCCTGTTACGTATCTGTATCATACACGTACTGATACTAATT
TACCTATCATGAACAATACCACGCAGGGTAGAGGCTATGGTGTAACTCTAGTAAATATAC
CAATGCTTCTTGTGATGTACCGTTACTGGTATCGATGGCAAGTTGAGAAGAATCCTGATG
AAGTTGAAGATACATATAGGTTTATAGGATCATTTGTATTGCCAAATATGGTTGACTCTT
ATTTAGATATTTCTTTCTTTAACAGGTTAGCAAGGAATGCTTTAGATATTAAAAATCCAA
CATTCCCTATACCGCATCCATTTTACATCACTGATATGAATCCACGTATTGATAAACTAT
GTACAACTATCAATAGAGAATCCATATTAAAGGGTGTAGACATGGAGGGTTTATCATGGA
TAACACCAGCTATCGTACAATCTAATTTGTTCGATATGATGCGACTCCCACGAGAACCTA
TTAATAGGAATAATGAATGGGCTTATGTATTGGCTCGCACACCCTTCATTAAGTATCTTG
TAGGACAGCTTTTAAAGAACACTGGTTATGATCAATCTTCTGTTAACGCTGTATTAATTG
ATCTTATAGAAGCATCTAATGATCAAGCATTTAAGCAACAAGCAAATAGCGAGTTTGTAA
AAGCTCAACAGGCACAAGTTGATTGGATGATAGATGCACTTAAAAGAAAAGAAATGTGAC
ATAAACCCCCTCCTAAATTGGAGGGGGGTTATATGCCGTTTTATTAGAAATTACCTTTCT
TCATTTTAGAAAGTAATTTTCTACGTTCAGCTCGGTTAATTCCTGGGATAGCTGGCAATA
GCATTTTATTACTAAACTTACCTGGTCGACGGTCATGATTTAGTAATTGATCAACTACAG
CTTGGTCACTGCTTTTAACTCCATTATCCATTTCATCTAATGGATCCATAATAGTTTTAG
CAGTTAGCCTTAGATGCGTAACCAGTTCATCGAATGAACTATCAGTATTAATAAGTTGTT
TATCAACACCTTCTTTAAGAGCTTGTTTAATAGTTACGTCTTTTACTAGAATATTATCAC
ATTCCCAAAGAGTTGTATGATGGGTATTTAGCATTAATTCAAGTGAGACATTATTGTAAT
CATTGTCAGCACTTGAGTGCCAATTGAATTCATCGATATAGTCATTTAGACTATTTAACT
TTGGTGGTTCGTTATTAGAGGTAATTAAACCAATTGGTTGAACATCATCTAGTGAACTTT
TATCACTAGTAATTGCAGTTGTTTCAGTAGTAACATCAGCTTCTTTTAATTCATTAGAAT
AAGTTACATATACCAATTCCTCAGTACCATCATCCAAAAGAACATAGGCTGTTGCTTTGG
TGGAATCATCAGGGGCTAATCCATGGATCCTAATACGATTATCATTTACTAATTTACTAT
AATCATTCCAAGTGATTACACGAACTTCATTAGTCTTTTCCATTATCATCTACCTCTTTT
AATTTATCTTTAATATATTTTCGGATAAAAGCTCGTTTCTCTTCACGAGTTAATCCCGAT
GGTACTGTTGGTAATGTGATAGTGCCGCTATTAATTGCCTTCTTCATCCGATCTAAGTCA
AAATTAAATTCGGTATGATTTGTCATTATATTGAACCATATTAAGTTAGTAATTGGCAAT
CAGACTATATAAATCTTAATTATTTTTTAGTATATAAGCCTCCCACTAGGGGAGGCTATA
TTTCTTAATAGAAGTCGGAGATGAGTCGATCGTTATTTTTATCAATAAGGAAAATACCTA
GGGACTCTAAGGTTAGATAGAATACACCCATAGTATTTCCAATGATAGTTCTAACGTCAG
CAACACGCGTAATAACTTCTGGAATACGACCAGTTTCTACTACTGTACTCGGGATATGGA
AATCACCAATACTGGTTTTATTTTCTTCAGTAACCCATGCTTGAATACGAGCAGCTAATT
TCTTATCTTCGATACCATCAATCCATTCTTTGATCGCAGTTTTATTATTAAGAGTAACTG
AGATCTTTACATGTGAATAAGGTGGATCACCAGCGTCACCAAATGATGGAGCAAATACAG
TCTTCCACACTAATCCCTTTTTATGGGTTGAGTTATCTTCAGATTTATAAGACTCAGGTC
GTTTAGTTTGACCACTTGTCAGATACTCAGCTTTACCACTTCTAACAGACTCAATAATTT
CTCTTTCGATATCACCAACTTCTTTTAGAATTGAAGCCAAGTCGATTTTCTCTTCAGTCT
TTACAGTTTTAATGATATCTTCCATGAGTGTTTTAGCACGACTATTAATCTTCTTAGGTA
CTTTACTATCCCTTAGTCCTACACCCTTGATCTCCATTCGTGACTCATTAAACATTACCC
CTTCTTGGGCATCCTGTGATGCAAAGTAATGTTTAGAACGAGTAGTTAAGGCCAGTACTG
CGAAATAATATTCGTTCTTCATTGCAAATAGACGAAGCTTATCTTTAGATACACCCATAT
TAGCGGATTGAATCGCCAAGATATGCATAACTACTTCAGACACTAGAAACACTAGTGCGA
ATACTAGTCGCTTAGCTTCATTGCTAAAAGTTACTTTACCAAAGAATTCCTCAACCCACC
ATTGTAATGTAAACATAGTGGAGTCAGTATCTGAAATAACAGCAGCTCGTCTATACACAG
TTGGAAAAGCATGAATGCTTGATGGTACACATTTAGTCAAGAAGAAAGCTTCAATCAATA
GACGATATTCATTAAGAGTTTCAGCGATATTTCGACCAGTGGCATAAACTTGGTTTAATG
TATCTGGATCTGACTCTTTTAGTTTAGCTTTAGATCGACCTTTAACAGTATCAAAGCAAA
TAAAGCTAGCCAATAGATCCATATCACCATCATAAGTTGAATATTCTTCTTCAGTGATGA
TTTGTTCTTTTGTTCCAACCTGGCTTAATTTAATTAGAAAGCTCTTGATTAATTCTTTAT
TGTATTTATATAAGTGATATAGATCACCTACGTACATAACTGCTGCTCGTTCGACTGGTG
TCATCCCAGTAGCTAGTTTTAAAATCTGCGCCGTGTAGTACTTATTTTGCCAATAGTGTT
TAGTTGAATAAAGAACCATATCAACTACTTCTTCTGGCGTTGGGCAATGTAGATTAAAAT
TAGTTACTGCTTTTTGAATTAACTCCATATCAGCTACATTAATAGTGTTTACTAAATTAG
CTTTTGTGATTTCTGGAGTATAATAATGCCTATTACCCATAATGAATTTTTCATTATTTG
AGTTAGCATAAGAGGTACCAGTTCTACAGGTAGATGTTAAACTAGAGTGTGTGGACTTAT
AATAAAGAATAGTGGCAGCAGATACAGTACCGCCTGAATATGAGTTATTGTTAATTTTGA
AGTTTTCTTGCTCGCCTTTACGTACTTGTGCAAGTTCTGTTTTAATTTGTGAATTCTCTT
TATCGCCGGCCTGTAGGAAAGCAGCTGCTTCACGTTCAGCCTGCATCTGTTCACCTTTAA
CACGCTTACGGTTTTTCACACCTTCGGCAATATAGATGGAGTGCGTAGACTGTCTAACAG
ACTCTGGTAAATATGCGGTCATTGATGGAGATAATAATAGATTCTGTTTCTTAACACGGT
TAAGAAAAGCCATAAATGAAACCGTCTTTAATTCCCTATCACCAAACTTATTTTTATCTA
GAATAGTTGCTAATGGATTACGAAGTGCATATTCACCATTTTGACGTAATTGTTCTTTAA
CAAATTCTTTACATTCTTCTAAACTAATATTTAATTCATCTTTTGTCATTAATTGAAGAT
ATTTGGCATTATCATCAAGACATGCATTAATGATATCAATATCACGATGGTAATCATTGA
CATCTTTTAAAAACGGATTTGGTTGGGCAAATGCGGTCATTTCACATATCTCTCAGTATA
CTTATTTTTTAATTATAGTGAATGATTTCTCTCTTTTTAACTAAAAAAGAAAAGGAAAGA
ATGGGTACCCTATACGGGTACCCTAAATTGAGAGGTAGCACATCAACTTACATTCACATT
AGATGATGTAAGTAAGTAAAAATAAAACAACATAAAAGGAGCCATAATGGCTCCTTTATA
GTTATTCATATGTCACTGAAGTTGGTGGTGCATTATTTTGTCGCACTGCCAATAGAATGC
GATCATACATTTTATTATCGACATCATCCCATACTAATGTCAGACGTTTACCATCTACAC
GTTCTAATGTATTCGCAACAATCCAAGGGATACCAATAAACATCACACTTTCATTATTGG
CCATTCGTACACGAATGTAATTGTATTGTGTTGGATCAACTGGAGCTGACCCTGCTGGTA
AACTTGGATATACTTGCTGCCCAGCAGCAGCTACATCAAAACCCATTGCAGATGCTACAG
ACGCAGTAACAATACCTTCGACTGTAACGTTCTTAAAATTAGTACCTAGGATAGCATATG
GATAGACCTCGAACGAGATCCGATCACCTGCTTGAATGTCTCGAATATTAACGGCCATGG
TAATACCCTAAAATAGTTGGTTACATAGGGTATTACTCATCTAGTAATACTACTACACCA
AACAGACCTTTCGTATCCATTGGTACTGTAATGAGTACATGTCCATCTGGATACTTTTCT
TTAAAGCTATTGATAAAGTCGGTGCAATATTCAATAACATATCCTTTAGTAGAATAATTC
TCTAACTCAATATCTTCATCAGGATCATCAAATGAGTCATGTCCATACATCAGCATATGG
ATGTATATGTCATTATTAAGCGTATCCATATAACTTAACTTACGTGATTCTTCAAATAAA
TCAAGTGGTGTTGGACAATCTGGATATATCATTCGCTTTGTATATTCATGAAGCAAATGT
TGCGAATATACTTCTGACTCTTTAGATTTAAATACGATAAATACTCGTTTTTCCATATCG
GTTCCTAGTAGGAAGCCTATGTGAACCGATAGGCTTTCCTATTTGCGTCAATTGTTAATA
TGTGTCACTTAATCCACATCATTGTAACAGTCCATACCAAAGTCTTCACCTTCTAGAATA
TAACCCTGATTATGTTGATCAACTGCTATTGATTGGATTCTTTCTACACCATATCCATTC
TGATAGAGATATGTTATTTCAGATTGTAGTTTACTAGCCAATGTTTTACACACAGTCGCA
AACCACATATTAAGAATACTCAATGCATATTTATCTTGCATAATCACAGGTGCTACTGAT
GGAAAGTATTCTTGTAAATAATAGTCAACCCCTATGATTGGTTGAGTAGGCTGTTGTGCT
AGAAATTCAATATATGCATTAATCGCCTGATACCATAACTGTTCTATAGACTGGTGTTCA
GTATATGGGTAAGGGGTATTGTAAGATTTATTGACAACTACATCAGCCATTTTATTAACA
ATGGGCCAGATATCACTGATGTTAATAACTACGAAACTCGTCAGTCCTTGTTGACCTGGA
GTTATGTTATTAGCATCATTTATTCGCTGCATTTCTTGTTGTCTATAAAGAAGTGGATGC
ATCATGTTTGAGACCTCATAACTAATTTAGTTACCATCTTATATTTATCTACTTCTATAC
ATTTAAGACTAGTAATAAGATTACCTGCTTGAATAACTTGTTTTTGGATTATCTCTAGGA
AATCAGTTCCAAATAAATCCAATACCCTTTCGATAATCTCAGCAGATCTTAAGTGCCAAT
TGATGGTATTAGGATTTCCCTGATGATCAAATACCATCATACTCGTCGGTCTATCTGGTT
CTATATAGGTATCGCTTAAGTAATCTTCTAACCATTGCTCTAAAAAAGTAGGCCAATCTA
ATCTCTGCCAATGGTTAAAGTATTCTACATATGCTGCTAGAAGTCTATTAATACACCACA
GTACAAAATCAGTCGGCTCATCTACACCTAGCTCTTCCGTAAAAGAACGCGGTTTCAAAA
TTAGCAACAATTGCATATTGACTCCACTCTTTACCAGTGACTATAGTTAATAGAACAACT
TTATTATCTATTACTCTAATGAACTTATAGTCTATTAAATTCTTCGGTGTTCTATGTAGT
AATTGGATTCCAGTTGTATAAAACAAATTAATGAACTCATCCCAATCTGGACCGTTTCCT
AATTGTATTGGTATATCCACAAAAGGAATAACCGTATTATCCTGTTGTAATTTAGACATC
ATCTTAACGACCCATTGAGTTAATAAGACGATAGCTGTAGCTTCATCCTTATTTGTTAGG
TAAGTAAATTTCTTTATCAATGTGATTAATTGTGGGAATACCACTTCGATTGGCAATACA
TATACCAGACGTTGTACTTCCTCCGATGTACAATCCATCTCGTGCTTCTGTTTCATACCA
CAGTTCCTTAGTATTTGTTGGAATGAGGTGCATGATCTGCTCAGCGCCCTCATCGAGAAG
TTCTATTAGTTTATGCATGAAATGAATGTCTTTAGCATATTCATAGAACATTTCTTCTTT
AACGACCATTTGCATAAGCTTCTGTTCTGCCAATGCAACAAGCAAATGCATTGCTTCGAT
ATAACTAATATCTAAAAAGTCTAATACAAATCTTAGATAGTTATCATCTTTAATAAGTTT
ACTTTTAGTCGCTCTTAGTAAAAAACGAGGTTGTGGTGTGAGTAATGGTGACGTATAGAT
CTTCTGGTTTCTCAGTATCATAAATAACCCTATTTAACTGGATGTTACCAGCTGATGGTA
GAACTACCCGGTTAGAACAATTATACAGATAAGTTGTTAAGTCAATATCACAAATATCTC
TATAACCCAGCATCTGATTAATTAGTTCTATGATATCATTAGATGTGCTACCTGATGCCC
TCTCAGACGCTGCTGCCAATACAATTGTTAATGCATATTCTGCACCAATACTATTTTTTA
TAGATTTCCAAATATCAGAATGTTGATCTTGTACAAAAGATCTTACTTCCATTCTGATAC
ACATTGATGTGTCTTTATCATTAGATATGTTCATTATAAAAACCTTATAATCGGTATAAA
GCCCCCCATAAGGAGGGCTTTATTTAGTATAGATTATTTTCTAAAATGTATCGCTCAGCT
TGCGCTCGCTGATCATCAGGTAAATAATCACCACGTTGATATTCAAATACTAATTTCTCA
ATAGGATAAGTTGCATTCTTATTCTTGTATACAGAAATTACATAGCTATGATACTCTAGT
TCATACTTAACATCATGCTCAATTCTGGTTGAAAAATATGCCATGTTAATAGAACTAACT
AGAGGGCTAATATATTTCTCTTCAAATAGTATTAAATTATCAAGTCCTAAATTTGCAAAG
AAATCATGGTTAACCTTAGGTATTAGATAAGATACTTTAATATTACTATATTGTTTAACA
ATTACACCAATAAGTAATGCAGTTAACTGTTCTTGAATATATTCTACTGCGATGTTATAT
GCATGGTTAGTAGTGATGTGGTCTTTATTTAAAGTATAGCTACTGGCACTAGTAGTAGAT
AGCTGAGTTTTACGCCTAGATTCACAGATGCTATAACCTAGTGCTTGTAACTTAATTTTG
TCAAAGCCATGGAAGAATGTACGGAGAAATTCACTAAATAAATAGTTTAATGGGTTCAGT
AGGTTAAGTGACACTACTGCATGATCATCGCCATTATCAATCCCATGGACACGATCATAT
TCTGCGATACGATAATCTTTGCCACTGATTAAAGTTATAACTGAACCAGCTTTAACTACA
TCCCATTGGATCCATGTACCACTATCGATATATTTAGCTACTTGTTTATAGATATCTAGA
TAGATAGTTTCTAGATAAGGTAGTTTCATTAGATTTTCTACCCAAGCAACTTTATTACTA
TCTACGTCAGTTACAGCACAATTAAAAATAGCTGCTGCCATCTTAAGATTGGTGTACTGG
TCTTTGGCTGTTTTATATGAAACTAAACTCTCTACTATATTTTCAACCATTAGATGTAAA
AACTGTTGAGCCACATCTTTACTCAATTTCACCCCAATCGCTTCATCAATTGAAGCTGAA
GCTGTAAGCTGACCTAGTAAATCGTATATTGCTTTTTCTTGGTGGCGTGTATCAAGTACA
ATGAGCTTATTCGACATTATATTTCTCCAATTAATTTTTTGGAATAGCGATCCAAACTGT
GTTCTTTAACCGATAGAATATTACAAACTTACCTGCATTTACTCTAGGTATAATTTTTAA
TATATCCCAAAGATCAGATGCGGCTTCATGATTATCATGAAACCATTGGATTTCTTGAGG
GGTATTGTTTATTGGGCTATATCCAGTAGTATACATGAATTCAAAAGAGTCAATAGAGTA
TTTAAAGAAGTCATCAATTAAGCTGTCCTCTACTGATTTAAATATATGCTCTAATGTACC
ATAGTCATCTGGATGTATTCCAAAGCTTTGGCACCTAATTGCTAAACGCGCATTTATTAG
CTCTTTTGGTGATCTTGGTACTAACTTGGCTATTACCCTCGTTACATCTATTACATACAG
GTCAGCTACTTGGGGAGTAGTCACAGTACAATCTCTCATTAAATGTTCAGGAACATGATA
TAGATCTAAAAATATTTTTAGTCAAATCTGTTCGTGGTAAGCCGATGTATATTATACCTT
AGTTGGGTATTATCTCCATCGAAAAAACGCGTACAGCGCATTATAGGACGTTTTTATGAA
TCCAATTACTAAGGCTTTACGTGATATTTCTTTTAAGATCCCAAAACAGATATTAAATAC
TGTTTTCCTATCTAGCGAAATGTCAGGCTGTGGTGCAGCTATCTCACTAGAAACCAGGAT
ACGCGAAGCTGTTATTGAACCACGTGTTATGTTAGATATTGATTTAGTCGGTGGTTCAAA
AGTATTCATCCCACTAGATTTCCCAGTGCAAGCAGAATATGTTGACCCTTATACAGTGGT
TTATTATATTCCAGACGAATACACTCAGCAACGCCCAATTATCCAATGTTACAGTATCCA
TTTTGGAGTATTAGGATTCCACACTGCTGGCTATGCTATGCACTATAACGAATCAAGTAT
GGGGGCATTAACACGACGTGTACTAGATTCAGCTAGACAGTTACCAGTTGCCCAAACAGC
ATATATCAACTTAATTAACCCACACACTGTCATGGTCAGGTATATCAATATTCCTAACTA
CTCATCATTCCTTGCTTGTCGCGTAGGTAATGATGAGGAGCTAAATACCATACGACCTAC
AGCCATACCTGCATTTTCAAAACTCATTGAGTATGCGGTTAAGTCATACATCTACAATGA
GTTATTTGTATCTATGGGTGAGGCACAGTTATCAGGTGGTGCTGAGTTAGGTGTATTCCG
TGATAAGGTTTATGAATATGCTGATGCTGAAGAACTATATCAGGAACAATTAATGCGTTG
GATGAAAATATCCAGGCAGTTCAATGATCCTGAAGGTAAGCGACACCATATTCGGACAAT
CACAGCCGCTCAATAAAAAAATAAAAAAAAAGACATAATGCCCTCCCATTGCGGGAGGGC
TTATGCCATTAAGATAATATCTAAATAACACTATATTTAAAAACATACTGGTATAACCCA
TCATCAATTAATACTGTTGAGAATCTTTTTGATGAGGTCACTAGGTGCAATACCAATGCC
TGAACAATACCGTCTAAAGTTATTACACGGTTGGCATGCTTTCTTTTCAACGATATTTAA
TTCAAAGCAAATATTACCACGTGAATCTAGAATGGATATAAATGCTTTGTTACCTGTTAC
GCGATATGTCTTACCGAATACAACAGTTGTTTTATATGCAAGGCTATTCATGATTATTCC
TTTAACATAGATTATTATCATGTTTGTAATATAGCCTTTAAATGTATTTAAATATTTATT
AAACCCCATAGCGATGAGCTATATATGTTTTTAATACCTCATACGCCTTATCAGTTGCCA
TGGAGCTATTGACAAAATCAGAATGATCAATCTTAATAATCTTACCAAATGAATCATAAT
CGATAGTTGTAACCACTATTCGATCACTATCACGAATCAATAGTTTATTTTTATTTAAAA
CAAAAATATTCCATTTCTCGCCATCGGCTAAAATAACATCCTCATCTTTAAGATTAAGAC
GAACAAACTGTTTTACATAAATACTTAACTGAGCAACTGTTGACATAATGATTTCTCGAT
ATCTGTTAATGAATTAATCATATCTTGGACATTAATGGTAATAACATTTTTATAAGTTAA
ATAAGTTGGATATGAACCCATAAATGTAATAGTCCCACAGATATGATTTTTATATCTAAT
ATCAATACACCCGTCATATCTCCAGATAAAATCAAATGATCCTTTAGTGTCGTGTTCAAG
TGTAAGAATAGAACCACTAACTAATTTAGGATATTGTTCAAATTCAGACATTACATAATC
TGCCAGTTCCTTAACCTCAGGATAATCAATCAATAATAGCATATACGTAAATCTCAATCA
GTTAATATAACACCCCATCCAGGGTGGACTCTTTCGAAAAATGGCAATGTATTATTTCGT
ATACTGTCTGCATCTATATTTGGAATGTGAATGGTACAACAGTGGGGGTTACTGTCAATC
TTGAAATAGAAATTATCTTTTAAATCTTCAACCTTGGATGTTTGCAATGGATATAAAATA
CCATTCCCCCAAACATACCAACGTCTATACAGCTTATCGTTTAACCGCTTCTCAATAACC
GAATGCGATTTGAAACGAGGAACTCGCAAACGCATCCCGTTTACTTCAGCCCATTTGTCT
ACCCGATCATAGCTAATAATTCCATCCTTAACCATTACAAAGATAAATCCCTCAACTATG
TTGGGGAGACGAACTACGTGTTTCAGAACCATAGTAAACGGAGATAACGTCAGATCAAGT
TCAGCTGCCATTTGCTCGTCGAGCTTTTTAGCAGTGTTCTTTGCCATCGGTTATCTCCTA
ATATACAGATTATTAATTATCGCTAGTTATTTAATATCTGCAAAATTCTGATTTACTTTA
CCCATAAAAATACCATCAGCTCTATCGTCATAATAAAGCTTCCCTGCTTTAGCATTACAT
AAAACTCCATAAATGTGACCATTGTAACCATCAAAATTAAATAAAGATGGATTATGCGGT
ACATGAATGAATCCCATTTTATCGCACAGAAACTTAGCTACAGCAGCCGATCGGCTAATA
CCTGCCTCACAGTGTACGAATAGGTGTTGATCCTTACCAAGAGCTTCACAGAACGAAATA
ATACGTTGTGCATGACGATGATCAAATAGATCATAAGAAGAATCAACATAATCCTCAATG
TCATCAACATTGATACGTAAATAATTAAGATGTTTTGCTGAATAAAGTGGGACTCTACCT
TTCTCCAGTAGACAGATCATATTTCTAGGGGAATAAAATGATTCAGCCACATGCAAAGGA
ATATAAGTAACTGTATTTACTGGCTTCATATTACACCTCGTTACAAAAAAAATAAAAGGA
GCCCCGAAGGGCTCCAGTATAAGTTAATCGAATACAAGACCACTACTAGTTGCTTGGTCA
TCCACATCTAGAGTAGACTGACGCTTACGCATATTAGCTTTGGCTCGATTCATCATTTCA
CGACGTTCTTCTAATCGTTTACGAATATCTTCAACAGATAGGCTAGTGATCACAAAATGC
ATTTGATCGAAATCATATTTCTCGGGATCGGCATAACCAGTAGTACGAGTTAAAACAGTG
TTAATGGATACTTCGCGATCAGGGTCAGTGTATAGTGAGGCAATAGAAATTGGATCCAGA
ATAGCCTGGGCTTCTTGACGATCAAAACACACGTGTAATTGGACAGTCTGCACAGGTACA
TCATGGTGCTTATGGAAGTGTGCCCAGTTAGTGATATCCATTAGATCAAGACTTTGGTGT
TCTTGATTAAATAGAGTTACTAGTGCATCTAGTACTTCAAGAATGTTTTGGTTCACCATT
GACTGCGGTACACCTTCAACATTCTCATGATAGTTAATAATCATCGGAACTTTACTAACA
GAGGAAACCCCTTCGAACGTCTTAAGGCTACCACTACTATTTCGAAGACTAATATCACTG
TCGTAAGATCCAATGACCACACTTACAACTACTTCACCTTGTTGTTGTAGATGACTGACA
AGGGAAGGACCAATAGTTGAACCAGAAGCACCACCCATTGAATACACTACAATGTTAGCA
TCCCCTGCTGGGAAATGATGGGCAATGGCAGGAATGTGTGCAGAGATTAGTTCTGCGGCT
GCCTTACGATCTTTACCCATACCGATAGCACGTTTACGCGCGGTCTGGTCAGCTAGCTTA
GTATCCGCTTCAATAATGATCGTATTATCGTCAGTATTATGTTTGTGTTTATTCTGTACA
CTGGTATCAATGTAACAAACATCCTCATGATAACCATGAAATAGTTCACCAATACGGAAA
CCAGCACCGCCACAAAAGTAAATTCGAGTTTTTACTTTAGACATCATTACACCTCATTGT
ATTTAATATAAATTCATATACCAACTTGTAAATGGTATCAGTGCTTAAAAGTAACGATCT
CTTCATGAGTTCGTTCTTGATGAACAATTAATTCATTAACCACTTTACCAATATCTGATT
TAAATTCAATTGCAAGCTTATCTCGACACATATAGGTTTGCCAAGTGCCACCAGACTTAA
CACAATCAATAATCATGTTAAGTACATTCTTAACTTCAATCTTACTGACTTTATAACGGT
AAATACTTAGAAGTTCAGTAAGCCATTTCGTTTTTGGGATTTTGGCATTTTTACGTTCTT
TAGAAATATCAAAAGGAATTAATGCAATGATCTCACCATCTTTGTTGGTGAATTCGAATA
CCATACCTTTTGTATTAGCTAAAGGACGAATATTGTAATTACCTTTAGCACTGCATACTT
CTTCAGATAACTTAACTAACCTATCTAGTTGAACATCAGATGCATGAATGTTATCTTCTT
TAAATGCTTTCTTTAGTTCATCATGAGTTAGTGGAGTACGTACTAGAGTGTAATCTTTCT
GTTCAGTCATTTTTCATTTTTTCCAATTTTTCTATTTTCAAATTAAACGGTATTGGCTTT
TTATAGCCAATCCCTTATTACTAAAAGTATTAGATTCTCGAACTAAGGATGGGTTATACC
CCTTCTCTAATAATAAGGAATATTATATGGGACCCCCCCAAAGGGTCCCCCCCACATAAT
ATACTATTTATATAATTTTCTTTTTAGAAGAAAAGAGATAAATAAGAGAAATACAGTCTA
TTAAAGTAATGAATTTCTCAAAAATTAAATAGTACACCGCGCGTCCTTTGCGTCGGATTG
GCCTGCCAGACGATGGTCCGGATGTGCGGTAATAGCGGGTTTTTCAAAAATACTTGCATC
GCAGTTGAATTGAGCGGTTGATTATAACGTGTGATCTTAATAGTAAATAGAACTTCTTCA
ACATCTGAATGACATCCTTCGAAACTAAAATATACTTCAGTCCGATCAATAAACTGATAC
ATTTCTACAGCTGGTTCAGCTGGATCTTTAAAGGCCAACTTATTAGCGAAATAATCAGTA
TGGGCTAGAATTAATTCTTTTACATTATTTGCATTCTTCTTATCAACAAGGAATTCAAAC
TCTTCAAGGTTATCTGTTTTGAAAATACCTTGATAAGATGACGACTCGATTAGTTCATCA
CTGATATCATCAACATGAACGCCCGGCATAAATTCAATGACTGGTAGAATAAGCTTGTTA
CCTCCTACGCCATCAGCGATATGAATTCCAATCACATTACCATCACTAATGTATTCGTCA
TCACCGCAAATAACGGCGCCGGCCGCTAGATATAAATTATCAACATATTTCAGATTATCG
GCAATAATGCGGGATAGCACATATTCTCGCACCATGGCATCATCTGGACTTGATTGCGGT
TTAAACGTATAGTGGGTGTTATGACTACTCATGGTCGCACATGTAACAAAAACTGAAATA
TCTACTGGATTAAAAATCCAATGAATAGAGATATCCTCTAATTTATGTGGATGATGTAAA
CCATCTAGCTTAGATAGGGCTTGGAGTTGTTCTTCCCAGGTAATGTTAAATTGAGTCATT
TTTTCTAACCTCTTCATTTATTAAAAATAATACACTTATTTTACTTTAGCAGTTGATACT
TCACATACATTAACTAATTGTTCAAATAACGGTTCGTTATCTGGATCATTAACTAATTTT
GATGTATTAATAATAATACCAGAATACAGCATATCGGTTACGTCTCGATCACTTGTTTTG
CTGGTTACACCTTCCCACTTCCATCCATAGTAAATTAAATCAAATGATGAACCAATGTTA
AAGATTAATTGAATAAATTTGCATGTACCATTTTCTTTTAACCAATCAGGTCTATATGGT
TCATTTTTATAAAATTCATGCATGAGCACTTTAAAATCAGTTTTAATGTTTTCATGTATA
ATGCAATCAATAGAATCAAGAGCTTCATCAAGAGCTGATTTAGACTCAATAATGATTTCT
GATGTATTGGTAGATTTTAGTGTAATCATACCTAGTTCATCTGAGTGTGCTCTTCCGATT
TGAAATACATTGAGCTTACCAATTGGTAAGGTGGCTTTTAATGGTAGGTTAGTATAGTGG
GATACATCACTTTTATAACTGTCTTCATATGACTCAATTAAATTAAGCCATGCTCTTTCA
ATACGTGTATTCTCAAAATGCTGTTCTTCTTCTAGTAATCTAGAAATTAGATGATTAATG
TATTGAACATTATGACCAGCGCCTGTAATTGAATTATATTTTGTATTACCGGTAATTCCC
CATTTATTATTTTCTTTATTTATCCAACCGTAGATTACACTGTTTCTAGGATATTTATTT
TTAAAATTATCGTATATTTCTTTTAGATGTGGATTAGGGGTAAAACTAAAAGTAGTTGAT
TCAGTCATTATAGATACCTATTTGAATATTGTTTATTATTGCAAATTAGTAATATAGATC
TTAAACTTTTTTTAACTCAGCATAAAGCCTCCCCTAGGGGAGGCAATATGTTCATTTATC
TATTTCAGCGTTTGGATCATACCAAGGTAATAAACTTAGTAATGCCGGTTGTTGTTTCTT
CATATCCTCTAAGATTGTTGCTTCACTTGCACCTTCTAACATAGTGGTGAAAGTAATCAT
GTTCTTAGAGTTATATATCGGGCGTAGACCATTACCAAAGAATTTCACCTGTTGAACCAT
TATTTTGAAATCAGGGTTATCGACTACATCCGGGATATAGATAGCAGGTGGTGCACTGAA
ATAACGTTTAATTAACTCAATAGCCCAATACATTGGTAAATCACCAAACTGTGATTTCAC
TGTAGCTAAGTTTAATTGGAATGCTTTAGGTTCTTCAGGTGGTGGTTCCACCGGATCAAC
TGGATCAGTGGGTTCTTCAGGGTCAGTAGGTTCAGTAGGCTCTTCAGGAGCCTCTGGATC
GCCTTCTGTTGGCTTTTCTTCAGATGTGTCACCACTTGGATCACCTGTATTACTTTCATC
TTCAGAAGGCTCCTCTGGCGTTTCTATGGGTGCTTCCGGAGTTTCCTGATTCTCCGACAG
ATCGGACTCTTCTGATTCCCCATTTCCAACTTCTTCAACAGGGCTTGGGTCTTCTGATTT
CTCATCTGTAGATGTGTCACTTGGGGTTGTAGTTTCTGAATCTGTCGTAATGTTTTCATT
TGCATTTGATTCAGTAGAAACAATTTCAGTAGGAGTGGTTTCAAGTTCATTGTCACTATC
CTCTACATTAGTTACTTCATTAGAAGGTTCACTAATTGCTGATAATGCCACAGGTGCACT
AGCTGCACGAGCTTTTCTTTTAGCTATGGTTGTACCTTAGATAGAATAGTATTTATGGTA
AGTGACTAATTCAGGATCAGTTAAATTGATACTTTGAATAGCCATATTCAGGATTACATA
GGAGTCCATTGTTCTACGGGTATATCCGGTTAATTCCATCTTGTTATATTTCTGTACATG
ATCTGGAACCCAATCACCAAAGATAACACCCAGTTTATGAAAACGATACACCAATCTGGA
TAGTGCACGTTGGGTTTCAGTCGGGAATATATCGGGAACATTTTCGGGAACAGTACATTT
ATCACTAAATGGAGTAATAATAGGACCACGATGAATGGTGTAGCTAAGCTTAAAGATAAC
AGCACCAAGTTCGTCATTAATACGCCTAAGTATACGATAACCATAATCGGTACGCTCAAG
TTTATATACATTACTAGGATCAGCATCTGCTAAACCTTCTAGCATGTCGTCTAGTGCACG
TTTGATACCACGGTTATAGATGAGTTCACGTTCTATGTAATCTCTCTCAAAAGCGGCCTG
AGGGCTTTCTTTCTCAAATTCCTTAGTTACTGACCAAGAAATTGTTTCACCAGTTAATTC
ATCAGTTAAAACATACAGATATGTCATAGAGTCAACGTATGAATACTGGATAGAGTATCG
TGATAAATCACATGGCTTAGCATTTAATCGTTCTTCTTTTAAATAGAAAAACATATCTTG
TAAATGATTATAAGTTTGGCAGCCACCACCAAGATGATCAATATCAATAGACGTATCTGG
CTTGACTGGAGCTTTGCAGCAACTCATTACACACCTCTACAATTATATTTGAGTTATTGT
ACTACAGATGATAAAAGAGAAAATACATCATAAGCCCTCTCCTTATGGAGAGGGCATTAT
ATTTACATAGTGTTAAAACACGCATTAAATTTAGATAATGAGCAGCCAGTATAAATTAAT
GATTGCTCTAGTTGTATGATGACATCGCCATTACTATCAAAATCAGCAGCAATAAAAGTA
GATTCAGTTATCTTAAAAGTAGATGGCAGCATTTCTAAATAATTAAATATTGCTTGATTA
TCGATGTCATGAGGATTTAAATCCATATGTTACCTATACATGGATAGTTATGGACCAAAT
ATTAACAGGACTGCATAAGACAATTGTGATTAAATCTCTATGAGTTCTTTGATAATGATT
GGAACACATAACTTTGATTAACTTTAATTTTTTAGTTTCTTTATTTTGAATTTCTTTGAT
AAGCGATTCAATAAGCTCATTAGTAATAAGTGAATGTCCATCAATAAATGGTTTTATACT
TACTAGCGAATCTTTGTTAAATAGAATTGCATCATCATATTCGCATGTAGCTGCGGTCTT
GTATATGGATGCAACTTTATTAACTTTGTCATTAGTGACTATAGAAAAATTAAATTCACT
CTTAGTTTTACTTAACGATGTTTCAAATAATTTATATTTATCAATAATAGTTTTATTATC
AATAAATGATTTCATCCCAATATTTAAATCTATGTCCAGTGTGTAAGAATCATGCATTAA
ACCAATTGATAGTTGAGTCTCATTACCACTATTGTTAAACTTAATATTATTAACTGAACA
TATTTGATTATTAATAAATGATAATAATAGTTTAGAAATAGTTTTAATAACATTAACTTC
ATAATCATTATTAGTACAAGCAAGTGCAGTTGTCATGTATCGACTAACTTGACTAACATC
TAGTTTCTGATAGTTACTACCACTATCTACCCATGGGTCAATATTTAAAAAAATCCTTAG
ACTAGCTAGAATATCAGAAGATGTTTCAGAATAAATGAAAAACACATTATTATTAAGATC
ATCTTTTAGCCAACCACTAGTTCTAAGATTTTCTAGATCATTTTCATTAGATACCAATTG
ACTTAATTGCTGAATTAAACTAGGGTTAGCTAGGTTTAATAAACTAATCATTTTATAAAC
CATTTTATTATCCTCTAATTTATGTAATTACATGGAATAAATAACATAAGTACTTTTATG
ACTTATGATTTGATTCTCCAGGAGGTTTTCCCAATGGCAACTATTAAAGAAGTCCTTGAT
AGAAATTTTAAGGATGTTCAATTTGATCGAGATTTATGTAAAAGAATTATTGACTTTACC
ATTAGTTTTATGAATAGGAATGCCGATCATTCTGCTTTCTTTGGCGGTGTATTACTAGGT
GTGCAACAAGTCAGATTCTTCGATACGGATCGCGAGATCTGGTATGATGATGTTTTACGA
ATCGACGAAGCATTGTTAGTTCAAGATTTTAAGTCTGTTGAATTTATTGATCCCAACCAT
CGTGTGATGTCAGATGTATTCAATCACCTTCCTGCCTATATTTGTTCTAGGTTATTGAAA
ACAACTAATGTTCCTTTGAATATTAGGCATGAAGCAATGGTTAGTTGTTTCATGGTATTG
CATTTTAAATATCTAACATCTTTACTTGTACCACGCTTTAAATATCCTGCACGTAAAGAA
GTAGCCGAAGCTGCATTCGCCGCGCTTAATTATCGATTTGACATTAAGACAATTGGGTCA
TGGGAGAAACTATTTAGGCAACGGGCCGAAGGTATTATTGCACCTGATTCTATTTATGCA
CCTTTCTTAACTGGTAAAACACAAGAACTGGATTATTGGTCTGGCCGTGTGGTATCTGAT
ACTCAAACACGTCTACGTGAATTAATCAACAAGTACTATGATGTTTATATCAGAACACTG
CAATCAGGTGGTAAATTAGTTATTTCATCAGACATGGCTGTCAATTCCGATGGTGAACAG
ATTCTACGTGATAAGTCTACCGGGTATCGTTCATATCTTACTTACATCCATCAAGTTGCG
CAACAAGAACAAAATTTCATTAGACCTGAGCTAGTTGGTATTATTGAAAAGATAATGCCC
ACAATGCCACCAGAAATGTTCATGGCGACACTTAGACACCTTTCCCGTAACATCGGCCAA
CCTAGGGCACAAAAGCTTGAGAAACTCGTGGATGAGTGCTTACTGTATGCTTTCGATTAT
ATGCAGTCATTACGTACAATGGTTGCTAGAAATAATGATCTACAAACATTGCTTGTAAAA
ATACGAGCAAAGATAATGGCATCTAAAACAGAAAATGCTCAAGTTATTTTCATGCGTGAA
GAAGGTGAGAAGTTAGTTAGGGATGCGACTAATTCTAGGGTACCTGCATATATCGCGGCA
ACTAGGACTGGGTTGATGTTGTATTTAATTCTACGTGCTATGACTAAGAATTACTATACA
AAAACAAAATAAACATAATGCCCTCCCGTAATGGGAGGGCTTATGCTGTTAAGTAACATC
AGCTAGATTGATCATTACATAAATTTTTCAGAGCATCGTAAATAGCCACATAAGTAAAAA
TAGTTAAACTATCTTTAATTTCCTTACGTGGGGTGCCACTGAATCCACCCCATGATACAT
TTGTATTACTAACACATACAGTGCTAATAGCTGGCTCATGATAGCCACCATCTACTTTAC
GCTTTGCATAAACCTTAGCCGTAATTTCTCCACGTAACTTTTTGAAGAAAACTAGAGCAC
CCGGAACGACTATACGTTTATTACCAATCTTCGTAATAAATTCTTTACGATTAGTAAAGT
GCTCTTTTAATAAATCATTAATAATTTTAGTTAAACTATCAATTTCATTCTTCATCGACA
CGTTGTTAGTTCCTTACAAATAAACTTGATAACGTGATCACTTACAAATTTCAATGGACC
AGAACAATTAGATTTCTCAATAGCGGTTTCAGTGTATGAAATTATTTGATTTCCACTTAA
GGATAATGAATCAAATAATTCATAAGAGTTATTATTTTTATTCTTAAAGGATACTGCATA
TTGACTATTTTCACTATTGATGCAAAATAGGTTAATAGCTGGGATAGATACAGAGTTATC
TGAAGTATGGCTATTTTCCGTAATATTCCAATTACTGTATACAGTGAAAATATTTGCAGC
TAGTAAAAGGTTGGCATTAACCGTTTCTAAACTAACATACTCCACAGAAGTACCTTTATG
CCAATATAGTTCAATTGGATTAACCCAAGTATACCCAATGGTTTTATAGAAGGCATGCAT
TACATATTCTCGCCAACGTGTGGGTTTCTTATTATCAGTTAAAACACACGTAATATTGCC
TGACGGATAAATGTGGTGGATGCTACATGCATTAATTTCATCTCTAACATTAGTTGTTTC
AGTAATATTAGAATGATCCTGTTTGAATAGCCATTTTAAATGACTATTAGTTGTGTCATC
ACCTTCCCCATTTGCAATGTCATTTTCTAACCACTTTTCAAATGTAGCTTGTCTATCAGG
TGTTGGCATTCCCCAATAGGTAGTTAAAATGAAACGATAATAGTATCGAATAATATCATC
ATCGTTAAGAATATTTTTATCCAGTTTCCAGTGATGTTGATACTCGTATACTTCGCTTGG
AATATTATTTACTTGATACCATGTATTAATTAATACTTGAATTGTTAATTGCCGAATACT
CTCACTTGTTAGAGTGTAATATTCCCTGCTATGCGGCTCTTCAATTGTCACTTCTTGATC
AGTATAACCAATGTATTTAGGTTTACCATCGATGAGTGCAAAATTAACATTCCATGATGT
ATCATTACTAAAAGTGATATTAATAAAACCTTGTTCAAAGCTAGGCTTCACTACGAAACT
GACATCATCCTTCTGATATGTCATTTGCGTTAGAATACCAAAATTAATTTTAGATGTAAA
TTCTTTATCGAAGTTATGTTTATAAAGTAATCCTAGTAAACAATCAATTTTATTAATAGT
GTGGTTTTCTAATAATAAATCAGTATTTGTAAGTTCATTAATTTTCATTTTAACCTCTCT
AAAATTTACAATCTAGTTTTACTTATTGAATAAGCAAACATGTAATATATATCTTAATTA
TTTTTTAGCATAGTTTTTAACAACATAAAGCCTCCCCGAAGGGAGGCGATATGCTTTAGC
TTTTTTGGTTAGTACCGACAGTCTGCTCATTCTTTTCAGTTTGAGCATTTCGCCGAAGTT
GACTAACCAGACCATTGCCAGCCGCAACTACATCGGGAGAAAAGAATGCATTGATATCAA
AGTTCTGATCTTGGTTGCGAGTCTTCCCAGGTGGGACCATCGGATATACTTTTGCCATTA
GCTTAATCCTACACCGTCGTAAGTTACCCGATTACCAACAGCACGTTCAATCTGGTCTTG
AATACCATTACGTGCACGTAGTACGTCAGCAGATAAACCACCCCAGTCTAATTTCTGGTT
ATTGGGGTTCATACCGCGAATGTTTAGTTTACGGAACATTTGACGAGCGTATTCTTGTAC
ACCTTCAGAAACATCAGTCAGTGCAGAGAACTCAACGTTTAGATCAAGGTTCTGACCAAT
CTGTGATGCATCTTTACGGTTTTCCCAAGGACCAGTAGTTAATGGGAACATGTTGGTGCA
AAGATAAGCGTTAACGCAATCCTGGAAGGTAGGATCAGGTTCTACGAATAGAACAGTCAT
ACCGTAGAAGGTAGCGTCGTATTTCTCAACCGGTACAATGCCATCTGATACAATACGCGG
TACTTTGGTATTCTCATCTGCAATACCATAGTTGATCCACCATTGTAAGAAACGTTGGAT
AGCGCGGTTTTGTAATTCCCAGCAACCAAAGTTTGGGTTAGACCGGGCACGGGTTACGTT
AGTAGCCGTTTGGATAACTTCACCAGAACCACCCCAGGGTGCTTCAGCGTTATCTACAGT
AAGCGTACGTTGTAAACCATCGATGGTACGAGTATGCGTTTCAACGAAAGCCTTAAGACA
TGCAACTAACCAGTTAGTATTACTAGCATACTTAAAGAATCGTGGAGCATCTAGTAGGAA
CGGCACTAAGTTACGTGCCACATATGGCGTGTTAGTAGCCAGGTTAGCTAAGTCAGGCCG
AAATACGTTAGTACCAGCCTGAGCAAGGTTTACAGTGTTACGGGCACCACCAGCACCATA
ACCAGTCTCGGGTGCCATCGGATCATTATAGCGAGCCATTTAACTTTTCCTCATTCTGAA
AGCCACCCACTCATTGCTGAGCAGGTGGGGTTTCAATACGAACAGTTTCCAGATTGAAGT
TCAACGTGGTCCGCGGGTTATTAGCCTCAACAGTTACGTTGCAAGTCCAGCTGGTGCCGT
TATTTGCGTCAATCGGAGTGATCTCTGTACGCGGGATAATGTTGACACGAGTACCGAACA
TATCACGTACTAGATCGAGAATATACTCGTCACAACGCTCAACTAATTGTTCAGGTGTTA
GTGTAGCATTACCGCTAAATTGAGCGTGTACTTTATGGATCAGTCGGATTAATACACAGC
AAATATTAACAGTAATCGGTGATAGAAGTACAGATGTATCATCTAGCATTACTGAACGTA
GGCACGGATAATAGGAGCTACGATGGTCATATGACTGACTCCAAGTAGCACCATTCGCCC
AAGCTTGTGCCCGTACACGATCATCAAAGAATTTAACATTTAGATCTTTAACAAAAGTAA
CACGGTTATTAGGACTTACATCCATTTCCATACCTGGAACTAGATTACCAGTTCCAGCAC
CTGCATAGCGTGCCCATGACATAGCTACGTCTAAAAGCTGCGGTACATATTTACGATAGG
TACCATCCATTAGTTTGCCAGATTGCATTACAATCATAGCGCGACAAACACCAGTTCCGT
ATAGAGTGGATTCTGGGAAAGCTTTTAGGCGAGTGATAATCTGTTGAACACGACTTAGTT
CAGTAGCTTCATCAGGTAGACGACTATCAGTTTCTACGAATGTAGTGAAGAAATATTGTA
GATCACGCCGCGCACTTAGTACGCGCATTGCCCGATATTTTGATTCCATCGGAAGGCCAG
TGTCATAAAGAACACCGAACTGATATTCTGCAATGTTATTATAGCGATCATTTAATTTGC
CAAAGTTAATATTTTCAATATCAACTAATTTGGCATATTCTTCTAGATCAGTTGTACCGT
CAGTACCACCACTAGCATAGATGTTACCATCTTTACCTAACGTAATGCCGCCATCTAGTG
GACCGAGTACTTGAATACCCTGGTAAGGATCACCATCAACAGCTAAGAAGGTTAGGAAGT
CAATTTCACCAGGTGCAGTAGTATGCGCAGCAGCGGCTGGGTTAACTCGCATCTCAGTGT
CATAAATCATCTGACGAACAAGATCAATGTTCTCATGATAGACGTAGAACTGAGAGAACG
GTGAATAAAGTGGACTTAGGCCAGAGACTACACCATCATCTGAGTAAGAATCAACTAATA
CATCACCAACATAAAGGTCAGCGTTATACATATCGCTGTAAACACCTTTATCAAATGTAA
TGTTTAGATAATCTTGCTGGTCAGCAGTTTTGACAATAACTGGACTAGTGCCTACTTCAG
GCTTCTCAATTAACTGAATACGGAATTGACGAGTTTTAAACTTCGCCATAGCTGCTTCAT
CGAACTCTTCGATATCAGCAGTTGTAGTACTCCATACACGCATACCGTTAGAATCACCTA
ATTTACCAAAGAAGGAAACCGGTGCTTCAAATAACGGATATACTAGTGATTGAGATCCAT
CTTTATCTGATACTAAAGTACCTGGTAGTACACGTTGTGTACCTACTTCAGAGGTATTAT
CTTCAATAAGAATAATACGTGCCTTTAGACCATCAACTTTATCAGCAGTCGGGACTGGTG
CATTACCAATGTCACGAACACTGTTTGGATAGTTGAATCCACTAAGACGACGAATCGTAA
GTGGGATTTCATCTTCCACAATTTCAATAGCAACGATTAGCCGTGATGGGTTGGCTGCAT
CTTCAGGACGTAGACGCTTAACATAAAAACCATTACCCCTACCTAATAGGTTAAGGGCTA
ATAGTGATTGTGTATTGAAAAACTTACTACGTGGATCAAGCGATGCTTGACCATAGATGG
ATGCAAAACCATCATCAGAATCACCGACATAAGTGGTTTCAGTCGGCCCTGTCTCAGTGA
AGAGACGTAATAGCGGACAGTGTTGTGCGAACGTGATGTCCGGACGGATCAGGGGCCGGC
GGCTACGATCCCGGATACCATTAAACACAACCCTAGGGACAGCGTTGTAATATGCCATCT
TTTAATTCTCCCAAGTTGGAGCTTTGAACTCGATGTTATGAGTGTTAACTAACAGTCAAT
CATAGATATTAATCATAGTTGACCATACTAGTTATTTTTTACTATCCATTTAAGGAGTAG
CGGTAATGTTTTTGCTACCGTATGAAACTACAGTTTGTAAAACTTTATACAATCCCACCG
GCGGTGGAAAATTATATCCTAAACAATATGTTGATCAAATTGAAAATGCGATCAAGAAAG
CCAATGTGTATCTACCTATTCCACCTGTTGATGCACGTAATGGTGAAACGCTAGAACATA
GTGGACAGATTACCCCAGTTGATGATTTTGAGGATATTAAGAAATTTACTCAAATTGTCA
ATATCGGTGATCGTGATAATCCTAAGCTAGTCGTTGATGCTCGTCTATATAAAAAGATTG
AACAACGTACTGGTATTCCTAGGATTATTCAGCAGAACGAATGGCAGTTCCAATATATTC
GGATGGCACTTAATATCAAACTATTACGTGAAGGCCCGGACTTCCTCCATCGCTTAGGTG
ATATCCCAGTTAAAGTTTTCTATAATTGGATCTCAGGTATCCTAACACAAAAATACAACC
TACCACCTGAATCAACCCAAGCTATTTGGGTAATCTGTGCTGTTTATTACTTTGCTATGC
AAGATGATGATCTAACAGAACCAGGTCAGGAACGTGATCGGTTAATACCAATTATTTCCC
GTCTTACATATATTCCAGCTGGTTTTATTGCGGATGTTATTGATACATTAGGTCCACTTC
ATAATGCCGGTGATCTAGCTTATGAGATTTCAACTAATGGTCGTTCAATCAGGATGGGTA
AACTAAAATTCAGTGATCTACAATTATTAGTATCACCGAGTTGGTTTGGTACCGCTTCCC
GTGAAAACGTAGGTGTGGCACTAGAACACATGCCAACTTACATCACACTGATCTACATGG
CATTAGCTGATCGCTCATACCGTAAAACAGTTTTAAGTCAGAAAGTTGAAATGATTTCAC
GTTCTGATGATGCAAGTCGTTTTATTAATCTAGTGAATGAAGCTGTAAGTAGCCAATTCG
TTAAGTAAATATAGGGGTGAATCAATGAACGCATATCTATTGCGGCATGCGATTGATAAC
GTTTGGTGTAACCCAGCCCAGGACAGACAGTTTGTTTATGAACTGAAACAGCTTACCCCA
CGCTACGGCGTGAGGGTAAACTGGGTGGTTGATTACACCCGGTATAAGCTACCAGTCCAA
AGTACACGTGATTATTGGCATCTTTATCAAATTGGTAAAATGATTCCTAAACACCTGGGC
TTGCCTAAGGTTTACAATAAGTGGATGAGCCTAAATGAATTGGCTCAAAACCATTTAAAT
TTAGCAGACGTTTATGTAAATAGTGGTATTAATTATTCACGTAATGATACATACGTTTTA
ATAACCAGTTCGCAAAATCTTTTAATTGCTGTTAAGATAGATCCATTATTCCCTGATCTC
GATGAAAATCAACCCTATCTTCATGTTTATAACAATGCTTACTTTCAATCAAATAGATCG
GATGTAGCTGGACATAGATGGTTAGTTTCTGAATCGTATCGAGTTAAAACAATATCTGAA
TTAACTCAATTTCAGATTAAGATAATGGATACCATAGCATCTAAAGGTGGTGTTCCTAAA
TACTTTGTAAATGGTAGATATGTTAATGAGATATCTCCTGTTACAGCAACAGTCGGTGAT
GTTTGTGATTTCATTCTAGATCCATCCATTAAAAGGATGGTAGATTTTGATCTACGTACA
TTACCTGTTTTCATGTCAGAAATAGATAGTGAAAGAAAATATATTTTACACTACACTGAT
AAGACTGTGCAAACAATTGAGTTCTTTGATGATGTTGAAGCATATATCTATCAGCCATTA
GGTAATAATCGTTATACTGGTGTTAATTACCATCATAACGAGAGCCGTTGGATGCGGATG
CTTACACATAAAGATTATTCTATACCAACTGCACGGATTGATCAGTTTAAAGCACTTCAT
CCAGAAGATCCTCGACGTGGTGCTGATCCTACTCGTTGGCCAAGTCAAAACTGGAAAGCA
TTAGATAATCTAGTATTTAGAATCTACATACATCATTCTGGTTATGATCGCCCATTAGTT
GCTGATTCACATCGTATTCAAATTCTGTATCGTTTAAAATCAGAAGATATCATAAGGGCT
ATGACTGGTGCAGATTCTGGTAATCCTTTATGGCGAGCTGAAAATCTAGAGCAATCACCA
TATTGCTGGTTCATGTCAGCACCATCTAGTTTCGTATACCCATTAACATTCAATCTACCT
GAAGAAACATCGCCTAGTAAGGTAGAAGCGCAGAATATGGCTGGTGATGTTTTTGGTTAT
TATGAAGCAGCTAATATTCAAGGTTATAATCCAGCTTGGGTTTATAATGATGCTGGTCTA
AAGACCGCTGATTTACGATACAACTACTGGCTAGATGCAACTGTATTTGAGTACGATGAG
AAAGGTATCTTATTAGGTTATAATTATCATACAGCAGGTCGCAAATATTTCCCTAAAGAT
AGTCGTTGTGCATATGTTGAATGCATTAATGGTAAAGGAAGTGTAGATCTACATGAAGCA
TATGGGAATGATCCCGTGCCTTTACGTGATGGTGACAACTGGCGAGTTTATGTTAGTCCT
GTTTGGGCTGGCGTACCAACTGGCGAATGGCAAGATATAACAGACCATCCAGATCGAAAC
AATTGGGGTTTTTATGATGATACCACTGATGATAAACGTTGGGTTTGGATAGCTAAGTCA
AATGAGTGGTATGGCCTAGTAAGAACCGATGAGTACTTCTATCTAAAAGAATTAAAGTTT
AATAAAACTGATGGTATCATTAAATGGAGTATACGCAATACTGAAACTCATAATGGTGTA
AAAGTCGATAAATTGATGGAGATACCATTTGGTCAGTATGATGTGTTTGTAAATGGTCGG
CCTATCATCGAAGGTCTTGATTACACGCGTGAATGGCCTCAAACTGTATTATGTAATCTG
GAATATTTAAATGCAGATCCAAATGCAGTTAATACGATTCTTCTACGTGGAACAGGTTTC
CCAACACCAGATTTAAAACCATACGAACCTGGCGAGATTGGTTTCATTGAGTATGGCGTA
TTGTCTAATGATGGTATTTATAAAGTACATTCAAATAAACAATCACGCATAATCATTGAT
GGTCATTATCGTGACCCTGCTGATCTTGAATTCCAAGAAGATCAAGGCACTACTGTTATC
ACTGATGAACGCAATGGTGCACCATTCCAAATACAAACACCACAGGCTCGCTTCCGCGAT
GTTTATAATGATGATTACCAAGCTAGGATTAAGGATGATGCACGGGATAAACAAGTCACT
GATTTTATGACTGAATATTTCCCAATGAAACCTCAACCTAATCCGGACAAGATCGATTAT
AGATACCAGGTGCTTTCAGCGTTTTCATGTAAGATCATTCATGATATCGTAAAAGAATAT
ATCAAACCCCCATATCAAAATGGACGGTATAGTGACGATGATATTGTTAAGCAGCTAAAA
GATTACGAGTGGTTAGCAGCTTATGACATTATCAATAAAGGCTACAACAAAAATAAAGTT
GTAGTTTATCCACATTGGTATACTGAACCTGTAGAACTAGATATTTTCCAATGGGAATAT
TTAAATCGTATTCTATCGATATATCTACGTGAAGTACCGCCACTATCCTTGTTCGTTAAG
ATTAAAAGGAATCAACCATGACAACGTCATATGAAAGTAGCCAGTACCAACCACCACAGC
ATAAAAACCATTTCTGGTTTAGAGGTGATATTGTCTCATATGCTGGTGAGACTGGCAAAG
CAATCCCTGCTAAAGGAGATTTAGTATTTGACGCAGCACAAGGTTGGTTTATTGTTCGTG
AAGTTGATGAAACAACTGGGGTATCTATCTTAGATCCATGGTACATGCCCCAAAAACCAG
GCAATGAAAATGAACAAAACCTACTAGTTGCTGTAGGTCCAGGATATAGCTCAGAATCTT
ATCGGTTATTCCTAGATCAGTCTGTAACACCATTTAATATTTGCCCAGACCGGCGATTAC
ATTTTTATGGATCAATGGTACATGGCTATAAAGTTTTCCTAGGTTCAGATATATCAGAAA
TACATGGTAAAGTGATTTCCCTGTTCTATGATAATGCTGGTAATTATCTAGGGCCAACTA
TACCAGTTGAATCAGTACCCGATCCATTGACTCAACAGAATGTTGTTAAAGCGTTAATGA
ATGGTAGGACTGCTGAGAAAATGCAAAATGGTGAACGTGTAACTCTAGTAGCTTATGATG
ATGTGGGTGGACCTGTTTCGATTGCTCAACTCGTTGTAATGAATACTGAAGTTATAGCGC
AAGAGGATACCTCTAAAAAATATGTAGGTGGTATCACTATTGAATCACCATTCATCTCAC
CAGCTGATCCAAAAGTTATTGAGTTCCCATTAAACGTACCAGTTGAATCATTACCGATGA
TGGGTGCTGTTCATTACCGTGATGGTAAGAAGCATGTGATGAATATTGATGGTACGGCAA
TGGCAATTTATGGTTTACGTAACTATATTGCCACTGAGGAAGGACAAGAGTTTAAATTAA
CTTTATCTTACCAATTAGCACAAGATGAGTTATCATACTTATCGACTCCTTCGGCTAACC
GTCGTATTCAGGAGACATATACAGCGCGTACCACGCCTGTACAGGGTGCTTACAGTTGCC
GTATGTTTGTTTATCCAGCTTGGGTTAATGAGGCAGTAGGTTACAGATTAGAATTCTGGT
TAGCCAATATTGATCGTCAACAAATTTGGAATATTACCCCATATGTTGAATTAGGTGCAA
ACTCAGCACCCTTTAACCCACGTGGTTATGGTACTATCCAAACACTAACATATGCGGTTA
ACTTAAACCAAGTTGATGGACGATTCCTACCAGTTCGATTTGCATCTACTTTCCAAGTAG
CACTATTGAGCGCTGGTAATAATCGGAATGCTAACTGGGAAATCTATTCACGTCCTGACC
AAGGCGAAGCATATGGTCGTGATCTTAAAGCCGATATCGAATTTATCAATGGTAATCTTT
GGGATCTTCGGTTAGCTAATGGCGCACAGTCACAAGCTGCCTGGCTTAAGAAAATGTACT
TTGCTGCTGAGCCATTAACTGGTCCAATGGAAGCTACTCCCCCAACACCTACGCATTTCC
GGGTGCGTACAGTGCATAACGAGTATGAGTATACGGTAAGTCAATGGAACACTGCATTGC
GCATTAATGCTCAAGATATGACCGATGGTGCTTTACTACAAATCACCTGGATTCGTCGTG
AGTATGATACCGACCTACAGTTAGCCATTACCGCATTACCTTGTTTACAACGTTAAATAT
AAAGCCCCCCTAGGAACCACCTAGGGGGGCTTTATAACGTCTTTTAACACATTATCCATA
TGAGACTATACTTTACAATTGCCGTTCAATACGGCTTTATTATGGCACATTAAAATAAGT
ACCTTAAAGGGCAGTGTATGGACGTTATACTTTTCAATAGTGATTGGGATAAATACTACA
GCGCTAATGTTGATCTTACTACTAAAAATAAATCTTTTATAAAGTTAGCTTTAACTTATA
AAAAGATGGGTATTAAAAATTACAAATTTATACTAGCTATATTGGACCAAGGTTTAATTG
GGGTGGATCCATATGACCCTAATCTTAGCGAAGAAATGAAGTTCCGTATTAACATGGAAT
GCAAATATAATCCTTGGTATTTTTTTAGGGAAGTGGCAAGAATCCCCCCTAACTCGGGTA
ATAATCCAATTCCATTTCAAGCTAACCGTGGTAATATTGCTTTATTCTGGTGTTATTTCA
ATCACGTAGATTTTGGTTTATTACAGCCTCGTCAGACAGGTAAGTCCGTATCAACTGACG
TGCTCAATACAGGCATGATGTATATCTGGGGTGAGAACACTAAGATTAACCTTATTACTA
AAGATAACAAACTACGCAATGCTAACATCGAGCGTCTAAAAGTAATGCGTGATTTGTTAC
CAGAGTATATTCACTATACTGATCCATTAGATGCGGATAACTCCGAATTGATGACATGTA
TTAGATTAGGTAATAGGTATCTAACAGCTGTTGGTCGAAATGATGTTAACGCAGCTGATA
AATTAGGTCGTGGTCTTACTGTACCAAATATGCACTTTGACGAACTTGCATATATTAACT
TAATTGGTGTTTCACTACCTGTTGCACTTGCGTCAGGTTCAGCAGCTCGTGATGAAGCTC
GCCGTGAGAACCAGCCTTATGGTAACATCTATACAACTACAGCTGGTAACATCACTACCC
GTGATGGTGAATTTGCATACCACTTCTTAACAGGTGGCTGCCCATGGTCAGAGGAATTCT
TTGATCTACCAGATCAGAAAACTCTACATCGTGTTGTAGAAAAAGGCACTACTGGAAAGA
AACCTCTAGTTTATGGTGCATTTAACCACCGTCAATTAGGACGTACCGATGAGTGGTTAT
ATAACACACTTCGTGAATCAGGTTCATTCGGTGAAATTGCCGATAGGGACTTCTTCAATA
TCTGGACAGTTGGTGGTGAAGGTTCACCCTTATCATCAGATGAGAAAGATAAACTTAAAA
ACAATATGCGTGAGCCAAGCTGGACAGAAATCACGGATGATGGTTATACACTTCGTTGGT
ATATACCAAAAGATGAAGTAGCCTCACGGATGATGAAGGGTAGGTTCGTTATGGGAACTG
ACCCATCCGAACTTCTTGGCGAAGATAATGATGCCACTGGCACAGTTGTAGTTGACGTAG
AAACACATGAGGTTATGTGTGTTGGTAGATACAATGAATCATCAGTGCCATCAATGGGTA
ATTTCTTTGCAACAATGCTATTGAGATATCCTAATATTCTTTGGATACCAGAACGTAAAT
CAATAGGTATATCGTTAATTGACCATGTTATCTTGATTCTACATACTAAAGGAGTAGATC
CATTCCGGCGTATCTTTAACAGAATTGTCAATGAATCATCAGAAAGAGAAAATGATTTCA
GAGACATTCAAACTCCGCTATCAGCAAGACAACCATCGTTCTATGATAGGTTTAAACGTT
ATTTTGGCTATGCAACGTCAGGTACTGGCGAGTATTCTCGTGATAATCTATTTAAGGTGG
CATTACCATCAGCAATGCATTATGGGGTAAGGACCATCTATGATAAACCACTTAGCACGG
AGTTATTAGCACTTACTATCCGTAATGGTAGAATTGACCATGCTAAGGGGAACCATGATG
ACTTAGTGGTATCATTATTATTAGCCCATTGGTTATTAATACAAGGTAAGAATTTATCTT
ATTATGGTATCAATGTTCCCATCTTAGGTAAATCAAAATTACGTGATAAAGAACCAAGTC
AACTTGAAAAATATCATGAAGAGAAAGAACAGCAAGGTCGTAAAGAATTTGAAGAGATAA
TTGAGCAGCTTCGCGGTGAAAAGAACCCGATGATTGCAGCTAAATTAGAAATGCGTTTGA
AACAATTGTCTAAACGTGTTAATATTGATGATAGCAGTGGTGTAGGTATTGATGCCATGT
TAAATCAAGCTCGTGCAGAGCGTACACGTAGAGTGCGTATTAACAGATATTCTAGAAATA
GTTGGTATTAAACAAAAAAAATAATAATACGTTACCCTCTTCGCAATGAAGAGGGTATTT
GAAGTTAAGTAACTCTAGCGAACAAAAGATTACTGGAGTTAGAATTAATATGATAGTTAG
ACGATCCGTCAATCCATTTAGCATCGGAAATCTTGTTTACAAGCTGACCATTGCCAATGT
AACCTAGGAAAGTTTTATTATCATATACAGCAATTTTGATTAGCTCATTTCGTCCGTAGA
CTAGTGGCTTACCATTGAATGCTGTTACTTCTAAACCAGACATGTATCCATAAATTGGAT
CAGGTCTGGAACTTGTTTCAAAGACTTTCATTCGAAGTCTCCTCTGCAAGAAAGCCCATT
TTGGGCAAAAAATGTCAAGCTTAAACTTGACGACATATAGCCCTCCTGGTGCGACAGGAG
GGCTAATCTTTTCAGAACGTACGGTGTTGTTATAATACTGCTAGCGAAAATACCCATGAT
AGGTAAGTAGCTAATCCGCAGTTCTCTTGGATGATACCTACTGACAGTGTAGTTGCAAAT
GTGCTGTTTGGAACCCCACTGGTTTAGCGGCCAGTGGGAATTCCTTTTATGCCGCTTTTT
CTTTCTTTATTGGATATTTCTTATATTTCCAAACCCACGTTCTTACGTTGGTAGGTATCT
TCCTTCTTATTATATTCGTAGTTCTCGATGAGACGCTTGTTGCTGAACTCGGACATCGAC
CGCATCTTGTCCATGATGGCCTCAGATATTTACCATGTTCATAAATTCGGCGTAGTTATA
GCCAGTATTCATGTCGAGGTAGTCTTGTTCGACCATGTCGTCATTCAGGTACTTGACGTA
AGCCTCGGCGTGAGCAGTGGCTACCGACTTGGTGAAGAAGTAACGAGCGTGTTGACGGTT
CTGTTCGAACCGACGCAGATTAGTAGTGGCCTGACCGCCGAAACCATTGACCTCCATTAC
CCCGTAAACAGTACGATCATCCAAAGTAGTGATCTGTGCAATCACTACACGAGAAGCATT
TTCCGAATGGTCGATGAACGTCATACTGTAAATCTCGATTTCTTTGAAGTCTTCCATTAG
AATTTCCTGTTAAGAAATGAATAGACAATCCCTACCCGAAGGGAGGGGCCTTATATCGTT
ACTTGCTGTGAAGATTTTTCTCGCGTACAGCGTCTGTTAACTGACGTGGCGGGTTGAGTT
GATCTGACAGGAGCTTCCGGAGGTTATCCAGACCGATTACTTTCTTCTTGGATATTGAGA
TTATTCTCTTTAAGTAATTTGTTGAGGTGGTATTTAACCTCCACCCATTCTAACCTATCC
ACGTCAATCCACTTCCGATAACCAGGTGCAGCAGGTTTGAGGACCTGGGTTTTCCAGCGC
TGAAATCGGAAGTTGCCGACACGGATATCGGTGCTCTCAACAAAGCGAGGTGGGAGTATA
CTGAGGTCAGTCAGTATCCCTCGTAGCTTGTCAGAGTTCATCAGTAAGGGCGACTTGAGT
TTCGAACACCGAAATGATCGATCATTCGTTGGAACAACTTGCCAAACGAGAACTTTTCAT
ACTCGCAGTCGATATGGAGGACATCAACGGTGTCCACCGGTGGATTATAACTACTGCTCA
TTGGCCATCTCCTTACGTTTGTTTTTGGCCCAGTTCTTTTCATAATCGGAACGGATGAAC
TTAGCGCCGGCGGCATTGAGCCAATAACGCTCCCCTTTGAAATAACGTTCCCGTTTAGCA
CGGGTCAAGCGAAGAGGATCGAACATATCTTGTTCCTGACACATGAATCGATTACGCAAG
AAGCAGTTATCATTCAGGATTCGAACCGAGTACAGTTGACCAAGCCGGCAGTTAGCCAAC
TTGGCTTTCTCGGTACGTAGCCGAGTTTCGTATTTCTTTGCATCTTCGTGATGATTGCGA
GGATGTTTCTTTCCAAGTTTCCTAGACATTATGTTTTTTCCTATTCTGTTGTTTTACGAA
AAATCTTATCATGGAACGTCATTGCCAACACGACGCCTATCGAACCACCTAGACCGCTGG
ATATGATAGCAGGAAGACCCACTATTACAAATAGTCCAATGGTGGTCAACTCAAGGGTAC
TGATACCCCATGAGGTGAAGAAAGCAGCTTTTTTGTACCCATGAAAGATGTTTCTTTGCT
GAAAGGTTTTTACAAAGGCATAGATGAATGATGTAGCTGTACTTAACAGTAGTAATATCC
AGATAGGTAGATCATGATTAATGTTAATCACTTCTCTTGTTCCCACACGGTGGGCTCACA
TTTAGTATGTTGGACATTATGAATATCTTCCAGTTGTTAGATATTGTGTTCCTTCGTATC
GATGGCCGCCAGCGCCACGATTTCGACATGATAAGGATGATCCACATTATGGCTGATACA
TGCCTCGCATTTGAAGATACGCCACATTCCATATGAAGGTAGTTCACGGTTTTTCAAATA
AGCATAATCGTGAGCGGCCGCAAGATATTTTTGTTGGATGGAAAACTCACAAGTAATGTA
ACCTTTCTCACGAATGAACTCATTGGTCATCTGATCGAAGATCTGTTTAAGTTCATCAGT
AAGGGCGACTTGAGTTTCAAACATTGAAATATTCCTTTTGATATATTAAATAGTTTTGAT
TATTTCGGAGTTATTGAATATTTAGGTTACCATCTGCTCTATATAGACTCGGCTGATTTC
ATTTAAGTAAGCGAGTGCCAATAGAATACCAGCCCAAATAACAATTTTTCGAGTAGTCTG
TTTTTCTATTACACCTGGGTGTGATTCACGAATACTACGGTGACAAAATACATAAAACAG
ACATAGACTCATTGTAACAAAGATAATGGTAAACCATCCCAGATAGATCATTTTCTTCAC
CTATTTTTTATAAATAGAGTTTATAGTTTAGTTAGGCGGATGATTTCTTCTAAAGCTGCA
TCAACTTGATAATACGGCCATGAATCAACACGAGGTTCGTTATCAAAGTATGTCCAAAGT
TGAATACCTTGGAGTTTATTGTAGAATACATTGATTCTGATTCCTTCATGGCCAACAAGA
CCTTCAATGTAAGCTGCACCATGCACATACGTAACATGTAGTGCGTACTCGCTATCTTCA
CCAATGTATTGTATAGGTCTGGAGTAGATGAAGGAATAAGGATTAAATGGATCAGCCTGA
GTGATATGTTCGAAGAATTCTTTCAAACTAGATTTTTTCATATTGGACATTATATTTTCC
TCATGATTTATTAAGTATACCTCCTAGAAGATAGACTTAATACTATTCGATGTTATTTTA
AGACACCTGCCTTGTAACCTTTAACAACAGTTCTAGCTTCTAATGCACTGATTTCGCCAA
TGTGTTTACGCAGTGCTTTAATAGCGTTGATGGTAGGTTCATTATCCGCGATATGTCGCC
AGTTTCCCTTACCCTTAATGAGTTCAACATCCTGGACATATAGACCATCTTTAACTAGAT
CCATTGTTTCTACCAACTTTTCGACTTCTTTAGTTAGATGCGTATGAAGACTGGCGAGGG
ACATGTCAGTCATTTGATGCACATCATAGAGAAAATCCCTATTATCAGAAGACGATAAAA
TTTCTTTGGTGAGTTTATTTACAGTGTCAATGTTTGCTGCAATTGAAACTAGCAGTGCAG
CATATTTCTCTGCATTATAATTCATTAATACGTATTCCTATTTAAAATTGGATTTAGTGT
AATGCGGTAATACTGCGAATATCTAATTCATGTAAATCATATTTCTTTAGGAACTCATTC
ACCGCTTGCTGAATGGTGTACCCACTCGGAGATTCCCACTGAGTACCATTAAAAGCAATA
ATACGATAACCTACCATTTTTTATTCCTCTAATCACAAAATAAATACCCTCCCGAAGGAG
GGTATTTATATCGTTACCAATTGATTACAGCGACAAACTCAAATACCCGACACAATTCTT
TATCACGATGGGCATGATGAAAAGATTTACCTTCAATACCGCAATACGGATAAAACAATT
TTCGGTGTTCCGTGTCGCCGAACCAGAAGGTGGTGTTATTGGAATCAACCACACCATGGA
TAGCCACATCACGAATGTACTGAAATACATGAACTGCCTTATCAGTGATCACTCGAAGTT
CTACACCGTGTTCCTTCATCATAGCCAGGATGTCTTCCTTGGTGTAATCTGCTTGTTCAG
CATTGGTCATGATCTTGTGAGTCAGAGAACGCTTTTCCTTGTCACCGAAACAGATACAGA
CCAATTGCACCAACTGACCTTCTACCTTGAGACCACTGGTCGGTTTTAGTAGAACGAACT
CATCATATTTGTAATCATTGGTGACAATCAGTGCCGACTTATTGCGAGTGACAATGCCTA
GGTTATACGACATAATTAATCTTCCTCATTTTAGAATATGTTGAACCTTGTGGATGTCTT
CCAGGCTATGACCAAGACCAGTGGCTTTGTAAGAATGTTCCTGATCAACACGCTTGTGAT
ATGCAAGCAGTGCATCTACTGGATGTTCAGCGAAGAAGTATTCAGTGTTGTTGCAGTCCC
AAGCGAACTCCCATTCACCATCACGAATCTCTATTTTGTTAGCACTGAGGTAAGTGATAT
TGATGGTATTTACAGTGGCATTACCACCAATGAGGTAGTACGATCTTTCATTGCTACGAA
CATCGATCCTGCCGCCATCGATAGTATCCTGATAGTAGAAACCACGGTCGAAGAATAACT
TCAGCATCGAAGTGAAGTAGCTGTTCAATACAGCTTGAGCATCTTTCTCTTCGACCACCC
ATTTTCCATCAACGATGTTATTCAGGTAATTACGATCATGATTGATCCCGACATTATCCA
CGATGTCAGCGTAGTTCTTATGGAACTTAGCAATCTCGTACAAACGTTCCAGAGCAGCAA
AACGGAAAGGGCTGGTGGGATGCAGAGCCAGAATGATCCGACCTACGCTCCACAAACCGG
TCCCGTTGACCTCGTAACTTTCTTCGAGGTAACCTTTGATACCACAGTTAACTTCAAGTT
CATCTGCTGGGAAACCGGTGCGGAGCCGCCTACGGATAGCCAATAGTACCGATTCGGATT
CTTCCGGATCGAACAGGTCTATGCCGAAAGTAATCCAGCTACCACGTACTTTGTGCTGGT
ATGCCAGGCGTACAGTATCACCGAATGGAGAAATGAACCAGCGTTCGTCTTCCGATACGA
CCAAATGGCAACCACATTGCCACAGGGTACTTAGAGGATCGTTAGAGGCGAGAATGCGCT
TGGTGTCGTTCATATCCGACGAAGTAGCATCTGGACTGCCACATAGGTGTGTCAGAATAA
ACAGAAGCGATGATTCTGTAGTTTGAGCACTCAACATTGCGCGAGTGATGGTTGCATTGA
TGATCATGAGATATCTTCCTTTTTGCAGTTTATGAATATATTGCTAAATAAAAAAAAATA
ATACGATGTAGAAAATAAAAGGGAGTCCGAAGACTCCCATTATTGCGTGTAATCTATTTG
ATAATAACCGCTTTAGAAAGAGTTTCCATACTTCCAATAACTAATTTGGATTTCTCTTCT
TCAGGGTTCTCCATTGCTTCATGCAGGAGTTCACCGATGGTTTTGGGTTGAACATCATCA
CTGATTTCACGCGAGCATGCCGAGTCGTCGTGCATATCAAACACATTCAGATCATCATCG
TCCAGTGGCGTGCTGTCATTCCATGAATAGCTTGATTCATCGGAAGAGTCCGAAGACTCA
CCGTACAGCTCATCGAAGTCATGTTCCATTTCTTTTTCATACATCTCGCTTATTGCTTTT
TCGTCTTCTTCTTGCGCCTTACGACGAGAAGTCTTGTAGAACGGCAGCAGGCGTTGCAGG
ACTTTATGGAACTTCTTCTTGCTGATCAACCAGTTAACCGAGTAGCGAGACAGCTTTACT
TCAGCTGCTTTGATGGTCGGCTCGAAATACAGCGCATTGACCAGCGCGTCCAGTTTGTAC
AGATGATCTTTAGACGAATTTGCATAAGCAGGATGTTCCTGAAGAGCGAACAACACAAAC
GAACGCATGTCAGTGATATAGTACTGACTATGATGTTTGTTGACTTCGACGTATGCCGGA
TCTACTACGCGGTTATAATCGAATTCCTCTGCCCATGTAAGGCTACGTACTTTAGTAGGA
CGAGGACCTTCTGCCCAACGAACAGTCGGATGTTTCTCACGGATGTTCCAACCATGATTG
CGATCATACTCGGTTGACTCTTCCTTGAACCAAGTGAAGTTACCTTCACCGCGCGACACA
ATACCGCGACACCAACTGACGTTGATTGCCTTGTTACGGCTTTCAGTAAGATCACAGGGC
ATGTCCAGAATAGCGACATAACTGTTATGACCATTGGCCACAACGCCGAGGACAGTTGCT
TCGAATGTCCCGTAGCGCTGGCCAGGATACACACGTTCGTTCTCATCCAGGTAACCAAGG
GTGCAGGATTCGAAGATAGCACGAGTACCGGCAGGGAAATGGGCACGACCGAAAGGTTGT
TCAGCCTTTCTTTCCTTACGACGGCTGATAACCCATTCCAGTTTTTCTTTGGACAGACGG
GTTACGGGTTCACGATATTTTTTCATGGTGTATTTTCCTTCTTACGATTAATTTGGATTA
TGTACATCCCAATGCATCCTTTCCAAGATACATTGGGATGGGGTCTCCGAAGAGACCCAT
TTTTTATTCCTGGGTCAGTTCTTTGACAGTGCCCTCGGGCGCTGGTTTATTCATATCACA
GAAGCTGAGAGAGAATTGGTAGCATGAAAGTCTTTATCTTATTGCGATACATATTCACCC
ATGCATTGGACTCGTCCACATGAGTGGATGTGTACTCAGCTACCTTAATATATTCTAAAA
AGCCAACAGCATCCGGACCTTGATCAATATAGACCATCTTGTTGAAAAGACCTACACCAA
TGAGCGATAAACGCTCTGCTTTTATGAAGGCGATGGTATCTTCGATGATAATCTTTTCCA
GGTCCTTGTACTTAACATGGTCGTTATGGATTAATGCCAACATGTCATCAACCTTTACAG
TCAACAACATGTCTTCATTACGACGGTTGACCAGTTCATTGGTCTGTTCTTGGGTGAACA
CATAGTCCACGTTTTGGAATACTCTCGACAGACGACTATCCATTTATTATTCCTCTATTT
AGTTACGAGCATTTCTTGACCATTGGGACCGATGCGATGACCGAGGATAACAGTTTCATT
AGAAATAGTTTGCTCTTCAAACTTAGCGTTTACAAACAGTCCGCCAAAAGTCAAACCAAT
AACCACTAGTGCGAATGCTTTCATTTTTAAAGTCCCTTTTTACATTAATTGAAGGTTAAA
TATCAAGTTGTAGTGGTTATTACATATCGTGCAGGTAGGTCTCATCAATGATGGCACGGG
CTAGCTCAATGAGGCTATCTTCATCAACAACGATGTTACCATCTTTCCAGGTAGCGCTAT
GGGATAACCAGTCACGGTAATCCTTACCTCGTACAGGATTGATGCATACCTGACGAGTAG
CTGCATCACGGTCGCCACGGAAACCCATGATAGCACGTGCACGACGGATTGCTTCTTTCT
CTAAGTCTTCATCAGATTTGAAAGTCAGGAAAGTTGCCATAGTTTGATTCCTTTTTACTG
ATTTAATAGGTTTAATTACACTGTTGTGATATACTGGTTAAAATGCTTTGAATGTATTAT
TTTATTTCCACAGACGACAATAGTCATCTACATGTCGAGTGATTTCTGCAACAGCCCTAA
GTCGCCGCCGACCAGTTAATTTAGAGCAGTGTTTATTAACTAATGTATCAACAGTTTTAC
GTAACCTAACGCAGCGTTCTTCGTTAGGTATATCTTGTTCGATAAACAAGCTAGTTAATT
TATGGATTTCCATATGGATTTTCTTACATACATCGTTAGCCGATTTTTCTTCATTTAATT
TGTTGTATGATTCGACTAACTTAACAATGGTTGTAGCAACAGTAGTTGCGGCAACAAAAA
TTTGAATACCATTGATAAAATTTTTATTAAACATTTTTAAACTCCAACTAATGTATTAAA
AAATATGTCTATATTGAAAATAAAGCCCTCCCCGAAGGGAGGATGTATGTTGCCAATAAG
TGTTACTAATTTATTTAGTTAGTTTGACCATACGTTCTTTCAAAGTTGTTTACATCATAA
CCTGAGTGGTGTGTTGCAGAACCACCTGAAGTAGTATCTGTAATCTTAACTACCACTTCA
TCATTTTCAATGTAAACCGACAGTTCATCTCGCATAAAATCCACGTGGTCATTTCGAGAA
CGTAGATATTTCATTACAGCTTCACCACTGATTTTACCGTCTGCCATTATTGTTTCCTCG
ATTGATTCCAGTTAAAATTATCTTTTGCATTTTTAGTGAAGAATTCGTTACCCCAACCAA
ATGAGTATCTAAAGTCCTTGATTACACCAACATCCGAATCGATAACCTTATATTCAATGA
CTTTATCACTATTCTCCAGAATGACTAAGATCGCGATAAGATCTTTAGGATCACCTACAG
TCAATTCATGTCTTTCAGACTCATCTCTGTGTATAACGATGTACATGGTACTTTATCCTC
TAATTACACCCATTTAGTTACTGGGAGTTCGAATGTTGCGCAAAGATCGAGTACTTTACG
ATGTTCCAAGTTATTTTCTGGATAGAAGGTAATGGGTGTAGTCGGACTACCCCAATTAAC
ACGACATCCAGTTTCTTTATGGATTTCACCAAGTGCGACATATGCACCTGCGCCATCTGC
ATAACCCATTTGATTAGGGTTAACACAGATTTGAGTAAAGCGCTCTGTCAATACTCGATT
AATAGTGATGGTCAGGAGACGAACGAACTCACGGGTCTTTTCGATGTCGTCTACCCAGAA
TTCCAGTAGGATCCATTTCCCATGAGGGTTATCATACCCACCTTGGAAGAATACACAGAA
ACCTTCTTTTGCTTTATAAAACTCGTGATTGGTGATGTTGCATTCAACTATTTGTTTCAC
TGCATGCCATACACGCTCGGTGACATAATCACCATAAAGTTCGATGCAGTGACCACGGCC
AGGGAATTGTTCTTTTTTAGAAAATTTAATTTCGAACATGGTATAGATTCCTTTTTACAT
TAATTGAAGGTTAAATATCAAGTTAGTAATATACTGTTTAAAATGGTTTGAATGTATTAT
CGGTAGAGGAGGGTTACCCTTTTAACTTTTTAATACTATTGGATAAGTACATGACACTAC
GGAAAATTTTGAATCTTGAAAAGTATTCACGTACTTCTTTTTTAGTCAATACAAGTCTTC
CATCTTTGTTTACATTATCACCAAAGACAATTTCCTGAGTATTCTTCCATAAAGATCTAT
AAAGATAACAGCCATGTGGTGCATCACACCAATCATAGATCTTAAGTTCAAATTCAACTT
TGTCGACAATGTCGTCATCAAATAAAATAGTATTTCCTTTACTATTTAACTCCATTAACT
CATTTGCACAATTTATTGCAAACTCAACCGCTGCCCAGGTTCTACCACCACTGAACATAT
CTTTACTTAATTTATTTAGATCGATAGCATTATAAGTTGTTTTCTTAGTGTTTTTCATGG
ACTGCACTCCTCAATAATAAATAGAATAATCTATATTTAATTCACCTTTGTAATATACCC
TTTAAATACATTTGAATACATGACATAAAGCCTTCCCCTGGGGGAAGGCTATTATTATGT
TTTAATCCAATGGGTATATGTACATAGGTCAGTTTCTTTAATAATGGTTGATTTCCATTT
ATCCCATTTAATGATTTCTTTAGGGAAATAAACACAACTACTATTATCGATTTCCATCTT
GACAGCAGTAATGTACATTTCGTCTAAAATATCCTTTTCAATGACTTCTTTATAAAGTTG
AGAACCGCCAATGATCCATGCATCCTTTCCAGTTTCTTTATTGAAATGAGTGGCATAAAC
AATAGCTGCACTTAGATTAGGGAGTACACCTAATTTATTATCATAGGCTACTGGGTAATT
ACCGTTACGATATAAACTTGATGATACAACAATGTTATTACGATTAGGTAACGGTTTACT
ACCTAAGGATAAGAAAGTATTTTTACCCATGATAACACAGCAACCAGTTGTCATTTCTTT
AAAGAAAGCTAAATCCTCAGGTATATGCCAAGGTAATAGATTATTATATCCAATGACACC
ATTTAGGTCATGGGCAACGATTAATTTAATAGCCATCAATTTTCCTTAGGCCAGAATTTA
CAAGATACTTCATTATCTTTAAATTCAATAGCCCAACTACAGCAAGTGAAATTAGTTTTA
AACTTAAATGTAGACGCTAATAGGTAATTAATTTGAGCCATTTGTAATAGTGTTAATTCA
CCATATACTTTCTCATCAATCAATACACCAGGTCTTTTATGTTTAATAACTAATTCATTC
AACTTAATATCTTGTTCAATATAGAGGTATTCCACTTCGCCTTTTTCTAGTATAGGTATG
TCCCAAAGAATCAGACTAATCTGTTGTGTGAATTCCTCAATACTAGTTGTAATATCTAGT
CCTTCATAACCTATCAATAGATTTATAAGTACTTGTAGACTAATCAGATTAACAGGTAAG
TCTTTTAAATCGTCTTCTTTAATAGTTGTTCTAAAATAAACAGTACCATCTAAGTTTTTA
ACTAATAATCTATTTAATAGATGATTGACTTCTGATTTAATCTGATAATCCAGTTTGATC
ATTAACTAATTCCTTGAGTCTAGTCATGCCATTGCTAACTTCAATAATGTAACTAAAATC
AAAACTATATTTCATCATTTTGAATTCTTCTTTAAAGTTAGCGATATTTCCAGACAGAGC
AATTTGTTCAAATCGACTAAAATCAGTGATAGTTTCATCGAACATTAGTTCTCTTGCGGT
AATATCTGGATCATAGTTAGGATGATCAACTACAGTGCCTTCGTGATTTAAAGTACCTTG
AAAATTCAAAGTCTTCTTAACATGGTCTACAGTTATTTTCCAGCTAGTTGCAATATATCG
AGCTTTAACTAAATTAGGAGCATACCTGTAAATACACCATTGTAGTGGAGTGCCAACAGT
TTGCGTATCATGCGTGGCTTTTATAACTTTAATAAAATTCTCAAGTTGATCAAATGTAAC
ATTTCTGGTTACACCATCACTAGTGGTTATATTAAGAGATGGTCGAAGACCAGTACCAAG
ATCATTGGTGATGATTATGTCCTCAACTAAAGCTTCATATAACTTTAGTCCTTCTTTTCT
GGATCCAACATCATTGCCCATTGTTTAGCTTCCTCTTTTGTTACCAGTTGATAAAGTTTA
TTAAATTCATGTTGACGCATTAATTTGTAATCTAATATACGCCCATTATTTTTACGAATT
AAAATAATAAAATCACCCGGACAGACAATTTTATCTTTATCTGAACCCACACGTAAAATA
CCATGACTAGATTCAGTTAGATTACATAATGGGCAAATCGCATTACTAAGAGTAGACTCT
TTAGTAGGTATAGCATAACCAACAATAGCACCTTGGTTGATATTATTGATAACGCCATCA
CCAGGTATATCACCGTTCTTTTTCCATTCGATTGCTTCAATTGGATCGGTGTATCTCGGT
AGATAAAAAGACATTCTTAATTAACTCCGATAAATAATAGTAGTATAGTAAAAAGCACTT
AGGTATATTTCTATTTTAAGTTATATTGCTGATAAAATAAAACCTAAGGGTACATAGTGG
GTTATTGTTTAAAGCTCTTAAATAGCATGGTAGAACGATATTGGACTATATAGCTGTAAC
CCTTAAGGCACTAAGGCTACAGAGGTTTTTAAGATAAAGATATCCAATGTACCATAATAT
GCTTTTACAATAGATCAACTAAATTCATTTAAAATGGGTCGTAAAAATACTTAGTTGATC
TAAAGATATGTTGAATACTTTTTCACATGAACTTACGACTCACAATTGGAAATTAACAAT
GTTAAAAACTATCATTAAACTAGACGGTACTGAAGAAGCATACTCACCTGCTAAGATTAA
TGGTTGGGGTGAATGGGCAGCCCAACATCTTGGCGATAAGGTGGATTGGAGTAGTGTTGT
GATGGATGCTGTTCAAGCTCTTGGTGATAAAACTTCATCACAAGAACTACAATTACAACT
TATTGAAGAATGTCTAAATCGTAAGACATGGTCTTATTATCTAATGGCTGGTCGACTATA
TGCGATTTATCTTCGTAAGAAGTTCTATGGTCTAAATGGCATCCCAACTGTTAAAGCGCT
TCAAACCAGGATGCGTAAAGATGGTATCATTGTTAAATTAGATTATAGTAGTAAAGAATA
CGCTCAGATTGAAAAGATCATTGATCATGATCTTGACCTACTTTGTCCGCATTTTTCACT
TCATCACATTCGTGGAAAGTATGCTCTACGTAATCGTAAAACTGGTCAAGAATATGAGAC
TGCCCAGTTTGTATATATGCGAATGGCAATGGCTCTAGCTGAAAAAGAGCCAGCTGAAAC
TCGCATGACCCATGTGGAGAATTACTATAAACTACTTTCTAATAAAATTCTTAGTGCCCC
AACACCTAACTACGTTAATCTAGGTACTAAGCTTCGTGGTTTTGCATCATGCTGCCTATT
TGCTTCTGGTGATAATGGCGTATCACTGGCAATGGGTGATTATATTGCCAACATCATGAC
CCAATCATCAGCAGGTATTGGTGTTAACTTAATGACTAGGTCAATTGGTGATCCTATCCG
TAATGGTCTAATCATTCACCAAGGTAAGAAACCATACATCGATGTAATGGGTAAAGCAGT
AAGGGCTAACCTACAGAATGGTCGAGGTGGTGCTGTTACGTGTTACTATAGTGCTTTCGA
TCCTGAAGCAGATATGATTACCCAGCTACGTAATCCACGTTCTACTGAGGATAGGAAGAA
CCGTGATCTTCACTATGCATTCCTAAGTAATAAGTTCTTTGCTAAGAAAGCGGCTCAGAA
AGATGGCATGATCTTTGTATTCAATCCATTTACTGCTCCAGATTTACATGATGCTTTCTA
TAGTGGCGATATTGATAAGTTTATTAAGCTTTATGAAAAATATGAAGCGGATCCTAAATT
TGAGAAAACTTATGTAAATGCTCGGGATCTTCTCAAATCAATGCTAGTTGAAGCATATGA
GACTGGAACCATCTATTCAGCTCAAATTGATGAACTCAATCATCATACGCCATTTAAAGA
ACCTATTTACAGTTCTAACCTATGCCTTGAAATCGCAGAACCCACTAAGCCTTACTATCG
AATGGAAGATCTTTATTCTAGTGAGGATCACGGGCGCGGCGAGATTGCTACTTGTTCACT
GGCTGCTATTGCAGTGGATAATGTTCCTGATAAGCAAACTTATGAAATGGCTGCTTACTA
CGCACTTAAGATGATTGATTATTGTATCCTTAATGCAGAGTATGCTTTCCCACACCTTGC
ACTAACCGCTAAGAATCGAATGAGCGCTGGTGTTGGTATCATGGGTCTAGCCACGCATAT
GGCACGTGCTGGTCTTAAATATAGTAGCGATGCTGGTAAAGCTGAAATCCACTTCATTGC
TGAACGGCATATGTACTTCCTCATTAAGGCATCACTTAAGATTTCTAAAGAACGCGGGAA
TGCGCCTTGGATTCATAAGACTAAATGGCCAGAGGGATGGACTCCACGTAAGACTTATAA
TAAGTCAGTGGATACTATCATCGAAGGTGGCTTTGAAGAACTTTATCCATGGGATGAGCT
AGAGAAAGAGATTAAGGAGAATGGTGGTATTGCACACTCCGTACTAGCTGCATACATGCC
TGGTGAGGCATCATCTAAAGCACTAGGGTCAACTAATGGTCCATATCCGGTACGTCGTCT
AATTCTGAATAAGACTGATAATGGCGCACGTGTGTTATGGGCTGCTCCATATGGAGATGA
TGATTCCTATGTGTATGAATCAGCTTATGATATCCCCACTAAAGATCTTATTGACTGCTA
TGCCATTATTCAAAAGTGGACTGATCAAACAATCAGTGCAGACCTCTATCGACGCATTGT
AGGTTCGGAAAAGATCTCTTCTAATGAAATGCTAAGTAATCACTTCTACATGGTGAAACG
TGGAATGAAAACCCGGTATTATGTAAATCTAGAAACAGCGGCAGGACTTGACATTAAATC
ACTTGAACGTGCTGTTGAGGTAACTAATGCTGAAGTTGGGTGTGCAGGCGGTTCGTGCAC
TCTTTAAGTGTATACACCCTCCCTTAATTGGGAGGGTGTTATTCCCAATTTATACTAACC
TCTATTATTTATTGTAAGAAATATTTTTAAATTGTAAAGGAACCGATATGTCTACTAAAT
CTCAACTACCAAAGAAAATCTTCAATGCTGCTAAGAGTGATTATCATCTACCGGAAATTA
TTCTTGGAGATGATCCAGGTCTACTAGATTCAATCCACACTCATTATCCTAAAATGTGGG
AGCTATATAAGCGTCTAAAGATGCTTGATTGGGATGAGCTAGAATTTGACTTTTCCACTT
GTCTAGTAGAATTTGAAACGTGTGATAAATCAACTTATGACATGATGATTAAGACACTGG
CCTGGCAATGGGAAGCTGACTCTGTAGCCAGTCGTTCCATTGTTAATATTCTATCACCTG
TCATGACAGATTCACGAGTATGGGCAGGATATGTACGTATTAATGATAATGAAGACGTAC
ATGCTTTAACTTATTCTGAAATTGTACGTAATAGCTTTAAAGATCCTAAAGTTATTCTAG
ATGAAATTCTTAGGGTAGAAGAAGCACAAGAACGGATGGTTGCAGTAGCCCGTACTATGG
GTGAGGCGCATGATGCAGTTCACGCGTATGCTCTTAATCAGGTACCCAATGATCAAGAAC
TTTACAATAAAGTATTCATGTTCTTCATCGCTCTGTATTTCCTAGAACGTATCCAGTTCA
TGGCATCCTTTGCAGTAACCTTTGCTATTGGTCGCACCGGTGCATTCCAGCAAATTGCAG
CTGCTGTTAAGAAGATTGCCCAAGACGAATTCGAAATCCATGCGCAATATGGTCAAGAAG
TTATTCGTGCACTACTGGCAACTGAACGTGGTAAACTCGCTTACAGTCAATGTAAAGATA
AAATCATTGAACTACTATGGGAAATTGTAAAGACTGAAGTTACCTGGGTTAATTATCTAT
TCTCTGAAGGTCGTGAACTGACTGGTGTTAATGCGACTAAACTTATTAACTGGGTACTTT
TCAATGCTAATGCCGCAGCAACATTCCTAAGTATTGAAAATGATGTTGTAGAACAGTATC
AAGTAGAGTTTAAAGAATCAGCTGGATTTGATTTTGTTTGGCCAGAGAAGAACCCACTTC
TTTATATGGAAGACTACCTAGACATTTCATCAACCCAAGCTTCTCCTCAAGAAGAAGAGA
AACCTGATTACATGGTCAACGTTGTGAATGATGTTGGTGAAGAAGAAGAATTCGAAGTAG
ATTTCATGTGATGAATAAATTAATTCTTGCACTATCCTTAGTACTCGTATCTATGACTCT
TGAAGCCAGTGAAATTAAAGCAACCACTAGTGGGATTATTAAACAAGTCGATTGCCAACT
TGTAGCTAAAGATAATAGTATCGCTGGTGCAACTATTGGAGGCACCGTCGGAGCTGCTGC
CGGTGCTGCGGTGGGTAAAGCACTTCTTGGTAAATCGGGTAGTTGGATTGGAGGTCTGAT
TGGTGGGGCAGCTGGCGGTGCTATCGGTAATAACATGGCTGCAACAGAAACTTACCAATG
TAAGATGATCATTGAAGATCATGGTAAACTGTACATGAATCAAATCGTCACAAATAAGAA
GTTAGAACCAGACATGTCTGTAACTATTGTAGAAATGTCTGATGGTACCTACGAAATAAT
GTAGACATAATGACCCTCCCTTAATTGGGAGGGTTTATGCTAACAATTCTATAGCACTCT
TATTAACAGTCATCAACGAGAGAGTAGACATGAATAAAATGCTAAACTTCCTAAACCGTA
CGCTATATAGCGGTACTGAAAAAGTATCTTCAAAAGCTACACCAAGTCTAGAACACTTTA
AAACAAATGTTGAACAAGTAGATAAAAAGATTCTACAACCCTTTAGTACTAAATTTAAAA
CCATTCTAAAAGAATGTTACAGTAATGAAGAGTGGGTTGAAGAACAATCATTTATTGAAG
AACCTATTGATCTTGGTTCAGCTGCACGCGGTCTTACCGAGCGCGGTATTATGCGTGGTG
ATTGGGGACGCTTAGCGCATTCCACTATTAAAGAAGCAGAAGGTATGATGCGTACTTATA
GTGGTCGTCTAAATGAAGATATGGAGGCATCTGAAATTAATGAAGTAATTCAAGATATGC
CTTATAACTTCACAGCTGGCTCAGCTAATACTAGCCGTTTAGAAGAAGATGATTCTATTT
TTGTTGAAGCAGATACAACTGTAGTTGAACCTCTGTCTAAGCAGACTCTGCCAAAAGTAG
CAGAACTTACTAACCAACTAGTAGAAGTTTATAACCGAATCACTGAAGAATTTACAGAAA
CTGGTATTGCTAAAGTTGAACAAGTTGAACAGCCAGCAGTTCTTGTAGCACTTGGTGAGA
TCATTAGTAGTTTTAATAAACTAATTGATGCTTCATGTGGTGCTCTACCAGTGGAAGAAA
CTGTTATTGTAGAGGAGGATCCGTTACCTGCCATTGTTACTGGTCCAACTACTGAACCCA
TTGATGGTGAAATTCCACCTGTTGATGCTATTAATAATTCTGCGGCATTAGAAGAGTTTA
TTGAAGAAGTACTAAGTACTAATCCAGAATTCATTAAATACCAAAGTATGAATGATAGTA
ATATTGATTCATATCTAACTGGAGATGACTGGATTATACTAAAATTTAAAGATGGTTCTT
ATTATTTATACAATGCCCAGAGTGCTGGTGAAACAAATATAGAAATCATGAAAGATATGG
CCGAAACTGGTAGTGGTCTTAATGGTTTTATAAATCGAGTTATTCGTGGCGGGTATGTAG
AGAAGTCCATCATTAATACTCCCGGTTTTATACAAGTCTCAAATGAAGGTCTTATCGACA
CCATTAAGAGTATCTTCGACTCTGGCCCTGAGATCAATCTTGATAAAAAGAAGAAAGCTA
TTGCTACACTGAAAAGTACCTATGGTGATGCCAAATGGGTGGCAGAAGCAAAGCTTAAAA
CCGGCGAAGTAAAAGTATCGAGTTACGAGACGTTCGCGTCTAATGATCCTAAGACCGCAA
AAGCCAATATCACTAATGCTGTAGCTAAAGCTGATCGTCACAACCAGAAAGTTCTGGATG
AGAACAGTAAGATACTAGCCCCCGTGGTTAATCTGTTCAAAGGGAATGATTGGCAAGACC
CTAAGAAAGTCGCTACTCTTCTGGCTGAATGGAAACCCAATGTAAAGACAAACTTCCAAG
AACCCAAATGGCCAGATAATGCTGGTGCTAAGATTAAGTTGCTTGATGCTACCGGTATTG
TGGAAGCAGCTAACGTTATCATTGACGTTCTGTCTAACAACACTAAGACTATTGCGGACA
CTTGGGTAGCCCAGTATCCTGACCTATCTGTCAATGGGGATATCTACAAACATAAGCTAT
CCACTATTAAAGATAAAGATGCTCTTGAGGATGTATATCTGCTGGCTAAAAGGGCCGATA
ATGCCATTCTTAACATTCATGCATCGTACGACGACGCTAAGGTTGAATCTACATATCGAG
TATTTGCTATCGACATGTTGAAACTAATGCAAGCATCTGTCAGTAGCTTCCCAAGTGGTT
CAACCGTAGAGAACACCAATGAACGCAATACGGTCATTGGTATTGAAGAATAACTTACTG
AGTATGTAATGATATGTAGAAATGATCCCTCCCCCTCGTTTCTACATTAGTCATAATAAA
TCGGAGCCAGTCCCCCTTCCAACTGGCTCCCAGAGAGTAAAACTCTTTGCTGGGCATTAA
TGACGATATATCGCCTCCCTTCGGGGAGGCTTTATATTTTGTTTTTACGTGAGTATGTTA
AAATATGTACAAATAACATAGAGTAATTATAAAATGATTAAGAATGAACTTTTACCAGGG
TTAATCTATGCCCAAAAAGAATTTGATAAAATTGCAGCTAATGTAAAAGACTATGATAAA
TATAAAAGACGTGAAGCTGGTAGGGCAAGCGCCGTTTTAAGAAGTCTAGTGAATAATATT
GTAAATCAAAATAAACCATCTTCACTTGAACATGAAGGTAAAGTTAGTACTACTAATACT
AATGAATATTTAGAAGAAGTTAATAATTACTTCTTTAACATTAATAATATTAAATTAATT
TCTCCTAAACTCATAAAAGAGAAATTAACAATTGATCTAATGAATATTTATGTTAAATGG
AATATGATTGGAGTGGCTGGCCGAAATGATGTGCCAATTATTGAGCACAGAATTAATGAT
TGGTGCGAAGTGACCGATGTCTACATTAATGGTAATAAGATAACTTCTTTACAATGGCCA
CGTTGAATTTAAAATAAGTTGTAATAAAATACCTAGTATTACATGTTATGTATTGAAGCA
CAATGCCCGAATGGTGAAATTGGTAAACACAGAAGACTTAAAATCTTCCGGCTACGGTCT
TGTCGGTTCGAATCCGACTTCGGGCACCAATTTAAATACGGAGTGTAGCGCAGTTGGTAG
CGCACCTGCTTTGGGAGCAGGATGTCGGGAGTTCGAGTCTCCCCACTCCGACCATTTTAA
TAATAGGTAAATAGGATGGATAATAAATGGATATCATGGGAACATCAAATTATAGGAACA
GCTCTTTACGCTATTCTTAGTGACCCTGAATTAACTAATATTCAATTAGCTCAAGGCTTA
CACTATCTAACAGAAGCAAAGTCTTCTGTATTGCATGTTTGTAATAATCATATTACATTC
ACTGTAACCTATCCACATGGCACATTTAGAACCAATGTAATTAGAGAGTGCCCTGTTAGT
GATACAAATACATTCAAATGGTCAGGTGTATTAGTCCGTCAAAAAGACGGAACATTCCTA
CCAGAATAAATAAAAGGGCCTATAGCTCAGTTGGTTAGAGCAGGCGACTCATAATCGCTT
GGTCGCAGGTTCAAGTCCTGCTGGGCCCACCATACACTAGCCTCCCACTTGGGGGAGGTT
TTATACTGTCTCATTGAGGAAAACATGAATACAGTAATAATGTTAGTATTATCTATCAAA
GTTGGATTATTTGGTTTCACTTCGACTAATGAGAGTAAGATCCTATTTGAAAATAGGGGA
CAATGTATTTCTCACCTGGATATGCTGGAGCATAAATACAAGTCTCTTGAAACTATTCGA
AATGAGAATACTCTGAAGATAGCCGACAGAGATAACCATTCGATTTATATTTTTAAATGT
CCCTAGGAAAAATATATTGAAACAACCATTAATCCGATTCTTAGGGGTGGTGCTAAATAC
CATTGAATTTTATTTCAAGCATACGGAGTAAATATGAATAATTTAACTGAATCTCAGTAT
AGTGTATATTCCACATTTTGTAGTCCAAAATTAATGTGCATGTCTCTTACAGATGCGTTT
ATTGAAATTCATAAACAACATCCAGAAGTAAAGCAATGTATTAAGAAAAAGATCAGTGAT
AGCGAAACTCAGTTCATCTTTATTTTTAAAGATGGGACTGATAATCTGATTATCAGTCGT
AAAACTGAACCGTGCCCTGAACTGGATATTCCCGTAGGGGTTAGGGTCAAGCTGTCTAGT
GAGGAACTTAAGAACATCCTTGCTGAACATGGTAGCCCTAAAGATGACAATTATTTTAAG
CACTGGACTGACCGACCATAATAAAAATATTACTGATTGTATAATACTATGTAAGAAGTC
ATGTCCATACGTTCGCGCTCATAGTTCAGTTGGTTAGAATACCCGCCTGTCACGCGGGTG
GTTAGGGGTTCGAGTCCCCTTGGGCGCGCCATTTAATTCCGTGATAGCTCAGTCGGTAGA
GCAAGTGACTTTTACGATTTCATTGGTAAGTACCCGAGTGGTAGCAGGGAGCGGACTGTT
AATCCGTTGGCGAAAGCCCACCGTAGGTTCGAATCCTACCTTACCAGCCAAATTCTAAAG
AGTAGCTTCGGCTACTCTTTTATGTTGCCATGGGTTATCTTATGAATTAAAATGATTTAC
TTGAGAGCACACTCATGTTTGAATTACTATTATCGCCAGATATAGGCGAAGAGTTACCCT
TGGTTGGATTTAATGAAATTATTAAATTAGGTGATCTACCTGTGGCACTAGCTGGTACTA
TGTCATATGTGGATGGAAATACACTATATGTTGGATCTGGACATCATACTGAAGGACAAA
CAGCCGCAACTGTATTCAGACGTTTTACCATATCACCATTCGCAGATGCAGGTGCAACAG
CTTCTGGAACATTCCTACCAGGTGTATCCTTAGGATTTGGAACATTACATAAGAATAACT
TTATTGTTTATGGCGGTATTACTGGATGGAACGCGGCGGGTAATGGTGGTACGGGGACAT
CTAACTTTATACAACATTTTGATATAAGTACAGGTAGCAGGGTTAAACGATATAGTGGGC
CTGTACCATTTTGGGGTACAGCATCCGCATCAGATGGTAATGATCTTATTTTGTGTGTAA
ACCCAGTTGGGGTAAATGCAATGCGGTTAAAACCATCGGGTAAGACATGGCTTAGTGGAC
AAGATTATTCAGGTGGTGCTCGTTCAGGTCAACAGTTATTCTTCTATAATGGTTACTTTT
ATCATTTTGGCGGCTGGGATAATACAAAGAACATACCTAATCTTGAAGTTTATCGATATA
ATGCAACTACCTTAGTATGGGAGCAAACACCTTGGATGATTATACCTGCTGATAAAGGAA
CAATCTGGCAAGGTAACGGTTATGTAGATGGAGAATATTTTAATTATATTAATGCAGTTG
ATGTCAATGGCGTAACTAAAATGTTTGCACAACGTTTTAACATTAGGCGTCGTAAATGGG
CTGAACCATTAGAACTTGGCATAGGGTTCCTTAATATATCGTCAATAGCTAAAGGTCCAG
ATGGCAGTATGATTATTGTTGGTGGATCTAAAACGGCAGTTGGTGGTGGAGCATATATGT
TGAAAAGCCAATTATTAACTGGTATATATCAAGTTAAAGTAGCCCCATTAGTTCTCGATT
AAACTAAAGATAAAAATACTAGTGCGATGATATTATGTAGGATTACTGTATCTAGATAAT
CCTAAAGCTATCCACTAAATACACTCGGTGGTAGATTTAATTATAGAGTGTGTCTGAGTG
CCAGGGGTTTGCCACCCCTGGCTATATTATGTCAATGAGTGTATCTCGTTCTTTATAACC
ACTATTGATATTTAATATAGGTGAAGGGAAGGTGACGAGGCCTTTCCATAGCCGGTATAT
AACCTAATTTAATAAAGGTTATATACTACATTGTAATAAAGGAACTTCTATAATGAATAA
CACAAATCAGTTTGTAAAAACTATTGTTGATTACCAGGCTATCCTGGATGCATTGATATA
TAAAACTAATGAGAATTGGAATAAGTATGGGATGCATTATCATCTTAATGATGTCAGTCA
TATCACTATACGTGATCATAGAACAGTAGGGTTTTTTATTGGTCGTCAATCTGGTAGTAC
TACAGCATTAATGGAATTTTCAAAACGCTACCCTGGTGAATGTTTGGCAGTATTCACCGA
CGAAAGAATTCGACGTGGTGCTCAAGATAAACTATCCAAAACACCAGATAACATTATTCC
ATATTTAACTACATTTAAACTACGTAGCTATATCCATAAATCTGGACTATCAACGATCCA
GAAAGACTTTAATGAAAATTTACTTTCAGATATCCGCTATATACTTGTAGATAATGCATC
TTATAATCTAAATTTACGAGGTATTACTGATAAAGAATTTAATCAGTGGGTAGCTGATAC
TTTTGGCACTAGTGTGATTGTAGTTCGTTTTAGTTAAAATATAAATAGGTAAAATAAAAG
CCCTCCGTCATGGAGGGCTTTATGTTGTTATGATATGACATTGTTGTCTAAATAAAATAT
AGTACGCATATGCGAAGTCATTTTTAATGGCTTTGCATATGTCATTAACTATAAATTATA
GTAATTTTTAGAAGAGGTATTTAAATGTTTGAGGCGTTATTATTTACACCACTTGTTAAT
TTAAAACCTGTTGTGGATGCTATAGACATATACGAAGGTAGTTTACCACCTTCAGCAAGT
TTCCAGGTTATCGATTTAGATAATACCGTGACTGAACCAGAGGTTATACCGGATGATAAT
TGAACTTAAATGGCTTGATCCTAATAAAACAAAATATCACTCCACTGAAATTTATCGAGC
CGATTACGAATTCGATAATATTGAAGATGCTGAATTAATTGCTACAGTTGGACCAGGGAT
AACATCTTATCAAGATACAACCGTGGAACATAAAAAAAACATATTATTATCGCTTTAGAT
TGATCTATGGATCACAAAAATTTGAAGCATCGGATATGTTCACATTTACAGCTATCCCAT
ACACTGGACCTGGTCCAGCTATTTTCCAATTTGGAGATGAACGTTTTGGTTATTTTGGAA
AGTTCCCAATGGACTTACCGTCATTACCATCGTTTAATATGGTTCGTGAAGCATTTAGTT
TAGGGCCAATTACAGGTAACTATCTAGAAGTAGCAGAACTTCATAAATTCGCAATAGGTG
GTACTGTTAGAGGTACCTTCCCATATCCATTGGCAATTGGGTCGGAACTACCAAGAGATA
GTGAAATCATGCAAACATTAATTAATGGTGGTGCAATTCCATTAACAATAGGTTTACATG
ATTGGAAAATAATAATACCCTCAGCCGAACATCATAATAATCCAAACCATAATGTGCAGC
ATTTCCCAGGTGAACTGCGGTCAATGATGGCAGTTATTACAGAGATGTATGCTCGTGTTG
AAGATGCTGAGACAGGTAACAAAACAGGTGGTAGACTTACATTAAACAGTGGTTTAAATA
TGTTCTATGACCCATTGAGTGCCAGATTTCCAGATACCGAAATAAAATATATAATTAGTT
CAGATTGGACTGATAATGAATGTACAGCTATTAATTGGACAAGTAGAGATGCAGTATATC
CATTAAGAGCAAAAGAACTACATACCGAAGTAGTTAATGTAGCCACAGATGACGTATGGA
ATAAAATGCTTATTTGGCCAATTCTAGTTTATACAGGTCTAACTAAACCGAAGGAGGTGT
AGCCATGAGAATTGAATTAACTGACTATAATTCAGATCCATCCACTACTATAAAGATATA
TCGGCATTATTTCCCATTTGATGCAGAAGATTTGCCGCCCGTACTACTTGTATTACCTGG
TAATACAAGTAGCTACACCGATGAGACCCTCATACTCAACACCATGGTTTATTATAGGGT
ATCAGTTGTACATAATGGCAATGAAGTAGTTGGTCCTTTATACACAACAATGAAACGTTA
TTATACTGGACCACAGATTGATAAGAATACCCCAGATAAAATCTTACGTGGTGATAGTTA
TATTGGTCGATATGGTACATTACCTTTGAACGTCATATTGTCAATAGATGAAGCTAAAAA
ATTAGTACCATCATGTACTGTACTTGAAGGAATCGATCATGAAACAATAGAGGTCGAGAA
GTGTAGTTATCATGGGAATGTGTTATTTATTTCTAATACACCTATTTTCTATGGTAGTCC
TGAAGATCTTTATAATGATGGTGCTCTACTATCATACGGAGACGGATTAGGTGTATTCAG
CCGCACATTTTACGATTCTTTAACAACTAAAGTTGAGCAAGGGAAACCTGTAACGTATAA
CAATTATTCATTTAAGTTCAGACTGATGACAACAAGCGAGTATGCTGAACTATATGTTAA
GTTATATCCAGATTCCCCTATGGGTCTTACATGTGATCGAGTTATAGGTAATTGTAACAC
GCATTCATATAAATCACCAGTCGTAGATATCGGATCACAGGGTAATAAATTCAGAACAGT
TGGATTAGATGGAGTGTCTTCAGACACAGAGTGGACTACTAAGGGACCACTTTATATGGT
GGTCGAGTTAATGAATAGAGGTGATATGGCATGGCCAAGCCCTGATGTAAATATCATTCC
TGCTGATCCAACTGATCGCATGATGTTCTGTCAATCTGAAATTATTAACGGACGTGCCCA
TTTCTTTGGTGGTGTTTATAGCCATTTTAATTCATATGCATCGGCCACTACTAAACATAT
CAGTATTAATTTGGATGGATCTGATAGGCAAGTACATGCGGATATGCCAGTTGGGGTATA
TAACGCATGTACTTGGGTTAACGGGAATAAAATCTACTGTATGGGCGGCGCCAAAAAGAT
AGGCAGTAGCGAATATACGTATGCTGAATTATACAATGACATTCAGGTATGGGAGGATGA
TGGTACGCCAGAAGGTTCTTGGTCAACTATAACGAATAGTTTACAATTTTATTATGGTGC
TAGTGTTACGAAGTATTATGATAAAATCACTGATCGTCAAAGACTTTTATATATTGGCGG
GTATGGTAATGTAATTCCTTCCGGACAGTATAATTTTGCAACAGCGGATCTGGATACATT
TGACGGTACATTTACAGTCTATAATCGGTCTAATTTCTACGACGGTGGCGGCGGTCCAAT
AGCACCACATGAAGATTATTTCTTATGGGTTGCTCCAGAAAGAAGTACTAATGGTTTTAC
AAATGCGACATATAAATTAACTATACCTGAAGACACTGACTCGTATACATATAGAACCAA
GATAACTCAACAAGGTACAGAATTACCAACCACCAAAGGTGGTAAAATTCATATTTGGCG
AGATACTATCTTCATGTATGGCTCGGCATCTCTTGATGAGAAACAAGGTGAGTTATACTT
ATATCAATTTGTACCTAATGAAAACCGCTGGTTGAAAATAACCGTAGCGATACCAGGATT
AAGTAATAGCAGTAGAGCATCTTTAAATGGAGCGTTTTACGAAGATAAGCTCGTGCTTAT
GCATAATCAACCTTGGCTTAATGAATCGTTTACATCAAGTTTCTATGTTATTCGATTAAT
CGATCCAATCGATGCACCAATCATCCCAGTCACAGATGTACAAGTTGGCACAAGTCGGAT
ATTTACAACTTATGCTGAGGTTTCGATATGATTACCTTTAAAATTAGGAATCCTAATTCT
AGATCAATTACAAGTATTGAAGTATACCGCGAGTTATCAGGTACACCTATTCCGGATACT
CCTATAAACCAACCAATCCAGAAACTAGACGGATTCACGACAGCTATTATTGATCGTGAT
GTGGTAATTGGTAATGTTTACGATTATCGCATTAGGACTGTAAGAGAAGGCGGCGACACA
GCCATTAGTCCAGTATTTACATTTAAAGCTGTTTCGCAATATGGACCAGGTGGTTTCTTA
GAACCTATGATGCAAGCAGATGGAGCTGCATTGTTAGATAGGGTAACTAGTCCGGAGCTT
CCTAGTCTTCAAGATCTATTAATGATGTTTAATGGGCAATCGGAAAATGCCATAAATTGG
ACTGATCAATGGACAAAAATTAAACATAAATCAGATATTTACTTTCTCCCATATATGGGT
TATATCGAGATACCGATAGACAAATTATCTGAGCTAATTAATAATGTAGATAATAATAGA
ACTATAGTTAAAGGTAATTGTAAATATTCTTTTTATGTCCCATCATTGCCAACTTCGTTC
TATATGCAAGTTATGGATGGTCGGTTTGTTGGACCATATACACTACCAGCTGATTACCCT
AGAAAATCAATGATCCGGTCAATTAGTGATCAGGTTAATAAAAATGCAGATGATGTCAGG
GTAATTATGACTGTAGTAGGTGCGAATTCAACTAGAGCATTTAAGGATCTGAGTAAGTAT
GGGTCATCAACCATTACCACTTTTAACCCAACTATTGATGCTAATGTTAGTTGGTATTGG
AAACCCATATTAAATTTTATCCAAGAATCAGGAGTAAATTAATGTACATCACTCTTAAAA
ATGTTAGAGGTTGGCCTGGTTCTACACCGATCGTTACATTTTGGTCGTATACCAATGCGA
TACCTTGGGATTACAATAACCTACCACCCCACTATTACTATGATTGGGATTTAATAGCAG
GATTCTGGCCATCCAGATACCCTGAAGATACATGCGTTTGGTTGAACTTAAAATATAAGG
ATATATTTGGTCAATTAGCTTATACAGGACCAATGCGTACTCTTAGTGTAAGTAGTAAAT
GGCAAAGTGTATTGGGATATACAAAGCTTGAAGCCGGTGATGGACAATATGCCAGGTTGA
CAAAGTCATACAAAGGTGAAAATAGACCAACCCTTAGACCAATTGGTTCTTCGCCATATA
GCATCCCAGGAATGAATCTTGATGAGGCTCCAACCCCTTATTTTATTGATAATGAGTATG
TAGCTTTTCATGATACATCTAGTATTGAGAAAGCTGTCGCTGTGGCTGTTTACCATGCGG
GGTACATGGGACGGTCTGGAGATATGTCCGATGTATGGCCGAGTGAATTGATAGAACATA
TAGGTGGTCCAACTCAACAAGGACGTATAATAGATATGCAGGACGTTAAGTGGGAGGTAT
ATGTTCCAACATTAAACGAATTCAAGCGTTATATGGGTACAGCTTTTCAGACAGGCATCC
AAACAGACCAGTCCCCGGTAACTAATTTATCACTGCCAGATAATGTAAAGTTCCTCACAT
CAACACCATCTCCAAGAAATAATACCATGTTAGTTGCTATCGATAACGATGGTATTGAAT
ATGATGTTGACATAGCTAATGAAGTTGATGTGATTTTTGTATTTAGACCTATACTAAAAT
AACTTTATACGGCATAAGACCCCTCTATATAGAGGGGTCTATTTATAAGTAAAAATATAC
ATAAAGTCATTATTATTTGTAGGTTAACCCTACATATAACTCAACTAGAAACCGGGCGTT
AATAAAATTTAACACTACGGAAAACATAAAGGTGACTCAAATGGCTTTTCATTTTAATAC
ACTAGATCTAGACGACTTTATTAATAATTCAAATAAAGTATTAAAAATAAATGTTGGCAA
GCAAACATTAGTAGCTTATGATGTTGATAAACTAAATATTGTTAATTCAGCTATTAATAA
ACTAGTTTATAGCGACCCTATAAGTGAAGAACATATCGAAGCATCACTGACAGATGATTC
ATTAAAAGAGCTTAATAATTATCTACTTAATAATGGCATTCAGAATAGCGCCAAGATGAT
GATTGATGCTGCTCTAGAAATCAAAGAATATTATAAAAATTACGATTCTTTTAGGCCATG
TATTTATAAATTAAAATCAATGCATACCAAAAATACAGATGATGTGTATCTAATATTTAT
TCTCGGTGAATCGGACAAGTTATTTGATACTATGCAAGGTTATATTGTTAATAAGTCAGG
TAATCTAATCAGAACTACTGTTCAGTCATGGCGATTCGAGAAAATTGAATTCAATGACTT
AGCCGAATATGATCAGCGTTGGTTATTTATTGAATCTTACACTAATGTTAAGCAAACGAA
AAATGATATAGCCAAAGAGCAAGAAGCCGAATTAACTAAATTTATTTATTATAGTACCCC
AAGTTAAAACAAATAAAAAAATAAATATAAGAGCTAGCCCATAATGGGCTAGCTCTATGT
TATCTTACTCTGCTACATCTTTTACATCGGGTTTATTATTTCCGCGTACTGCTCTCACGA
TACCATACGTAAGAATAAGAGCACCTATGGTGCCAATAGTGGCGCTAACAATAATACCAA
GAGTTTTTGGATCAATATTCATATCTAGTACCCTATTTACAATTAATAGAAAATATTACT
TTTAGTAATATCACATTAGTAATATACTGTTTAAAATGGTTTGAATATAACTTTATCTAT
TATAATAAAATAATACTTTCATATACACTATATGTAGAAGATTACGCCGCTATAGCTCAG
CTAGGTAGAGCAACGCACTTGTAATGCGTAGGTCCTCCGTTCGATTCGGAGTGGCGGCAC
CAAATTTACTGAGGTTTAAACTATTCTTAAATATATTAGTTAGGATGGGATTGAACGCGA
AAGACTCAGTAAATCATATTGCCCCTCCATATGGAGGGGCTTATGTTTGTCATTAATCAG
GAAAGTATAAAATGAGTCATCTATTATTTATCATCCAAGAATATATTACTAATAAATTTG
AGATAACACGAATTGATATGAAGCCATGCAATAGAATGTTACGCGTATGTTTATACGGTC
AACATGAGGGAAAAGGTTTCGTCCGTGTAGATTTATGGTCAGTTGGTTATCGTATTACAA
AGAAATAATACTCCAGATTATTTAATATGTATTAATCAAAGGAGTTTATAATGAGTAATG
AAACTAACTATCTAGGTTATGAATGGAAAACAGATATAACTACTTCAAATCTTAATAGAG
TAGTTGATTTATATACACTTGAATTAGCATGGCTAAAAGAAGATTTTAATGATACTCTTT
TTATAAAGTCTTATAAAGCACTAGAGGGTCTATTAGAGGAACCGTCTAGGGCAATCCATG
ATGATACAGTGCCCATTCAAGATCAATTAGATGAATTAAATACTGTTTTTAAATTAGTAT
TTGGGAAAGACAATGATGTAGAGTTATCAATTAATGATGATTCAATTATTGTAATCGGCA
CCACAGATGAAACTAAAGAAAAGTTAGAGGTAGAGGTGCGTGAGTTTGCATATAGAAAAT
CATTAATTGATGAACGTTATCCAGACATTGTAACGGATTAAAATACTTACAGCTATCTAG
TATGTAACTAGGCGGATTGCCATTTGTAAATTATCTATTTAATCAAACTGAGGAAATACT
AATGAAACAATTCTTTCAACTACTTCTAAGCCTACTTTTCAAACTACCGGTTCTATCATA
TTTTGCTGAGAAGAAACGACTAGAGAAAGAGAAAAAGGAAGAAGAAAAGCGTCAGCAAGA
ACAACGTCAAAAAGAACTACTTGACGAACAACGCCGCGAACAAGAAGATCATTATCGAAA
GACAGCTTACGATCGCCTAGCAAAACTTATTCATACTCGGTGGTATGATGAGTTTAATGC
ATATGAAAAGAAACTAGTTGATCTCGCTGTATCGAGTGGTAAAGCAGTTAGTGTTAAGTA
TGGTAAAGTCACTAAGATGCAACACCCTCAGCAATTTAAACTACTTAATGATTGGCTGGA
TGATATCCCAGTAGAAGATTATTCTAAGTGAGTTTAAATAAAAAAATAAAGACATAGCCC
CTCTCCACATGGAGAGGGGTTTATGCCGTAATTATACACTTACTTTGATAAGATTTTTAA
TATCAGTAAAATGGGTATATGTTGCCTTTTTATCTTTGTGAACCAGTAAGCAACAGGTAC
CTTCATTTACTGTATTGGTTTGTTTTAATGGGATATTTAAAGCATATTCAAACAGATCAT
CCATCCAGTTAAATACCATGAACTTATTGATAAGGACATGATCGAAATCAATTAATCCAA
TAAACGAATCGATCTCAGATTGATAATACTCTACAACCATTTTAGACCATTCTTTGTTTA
TCGCCTCAATGACATTAGGATTATTTATTTTATTAAATAACTCAATACGTTCCTGAGTAG
GTAAACTGAGGTAATAATTAATATTACTCCTAACGATATCTGGATTAGTGATATCCATTT
TTTCTGGTAAGTTATATTTACTATCTACATAATTCTTAGTTTCTGGTGTCATTCTCCAGC
AACCATAATCAGCAGTAGTACTTGATGGATCTATTTTAGTCTCCACTACTTTAGAAAATA
CATCTACATATAAATTAACAGATTCGTATACATCAGGTCTGATTGGATTAAAGTTTAAAC
TTTTAATAAGACCCCTAACTGGCCGAGTACCAATTAGACAATGCAATTCAAGCTTAACAT
TTTCCTCTTTAGCCATCTTAAATATATCAGCAAAACTGAGCTTTTCTGCCATTTAGAATA
TACCTTTTATTAATTGGACAAATAGATTAGATACACATAGCAATAGAATAAACCATAAAC
CTAGTCTTTGTAATTTATCTATACGCTTAGCTTTTTGTTTCCTCTTTAATCTAACTAATA
ATGTATTTTTAATATCATTCATTAAATTATCTCAGATTTTTTAGACGGTGTATAAACCCT
CAGTATTACATGTTGTTATGATATTGTAAAGACGATGCCAATAGCGATCATACGTATTTT
TACGTTTGTAGCTATTGGTGCGGATGACTAGATTGGCGACCCAGTGATTCTGATCAATGG
TCTCATGTAGATGCCCGAAATAAGCCCCCAGTTCAATTTCGAATGGGATCTTACACTCAC
GCCCCTTACACATTGCTTCACGCAATAAACGCATTGCAGAATTCTCTGCATTGGTATCTT
TAAATAAAAGACTAATGTATCCAGGGCTACCACCAGTTTCTTCATCAACATGGCCTGAAC
AGCAATAAATAGTTGCAACATTGATAAATTGATTAAACCATTTAATTAATGGCCTACATG
CTTCATCAATAAGATCTTCATATTCAGGTTTATACATTACTTTAACAGTATCATCTGCAC
GTTGTTTCACATGATGAAAATATTGCTCCCAATTTACATTAGTGTAATATAGCATATCCA
TATTAGAATTACTCATACAAATCAACCCCATATGTTATTGGCATATTCGCGGTCATTGAA
AATGCGGGTAAGATCCATCCGCTTTTGAATGCGCGCTAGTATCCATTTTAGTTGTTTAGT
TGATAGTTCTTGATTTTGCTTGTACTCACCTGTAGCATGGATGTAATAATCAAATTTACG
ACTTACAGTAGTAGTATGTGCACATGCAATAGCAAATGCTTCAAGTGCAGTTAGTTTGGT
TGATCCATTATCATTACAATTAGGGATAAACTCAAGTACACTATTAACTGCATCTACACC
ACTACCCAAATGAGCCCAATCTTTCTTGGTGATGTTGATACCCATGTCTTTAAGAATAAC
TGTATATATGTCACCATTCTCCATAGTGAACATCATAGAAGTATTACCATTAAACAACCT
GCGTTCTATGACTGGCAAGATATTTACTTCAACTTTAAACCAATCAGCTAAATCAACACC
TGCGGTAAGGGCATTCAATAGATCATAAACATTATCTATAGTTCCAGCAAAAGCCATAGT
TTTAACACGTTTACCTTGCCAAGTAACTGTCGGATGAAAAATTATCTTGTGGCAATCATT
GTAATATACATCTACACCTTCAGCTTTAGCAGTTAGATCAATATAACCGTTATCTAATAC
TGGAAGTCGCCCAATGCTATCAGTTGCATTATTCCGCTCAATTAATAGGCAGTCTGCAAT
CATGACTTTACCATCATTTACGATATATGTCATTTTCACTCCTTATAATAAAATAAATAG
GCTTAAGCGAAATAAAGCCCTCCCCGAAGGGAGGGCATTATCAATAGGTGGGATCTCGGC
GGCACCTTCATTTGCGGACGGGAACGCTGAAGGGAGATAGTCCGGGAGATCCCGATTTGG
GTATTCTTTACATATGGTATTACTGCAAGTATTTGTTCCCCGCAGTATAATCACCATTGA
TCCAATTTTTGATTACAACACTGATCCCTTCATTTTCTTTAACGGTAACCCCCAACTCAA
GATCGATTGATTGATAAGCTACAGCCGCCAGTGTTAACCGAAGATAAACGGATCCATCTT
TTTCGATATCGTAGGATACTACGAACCCGTCTTGTAGATTTTGTAGCTGGAATGTATTAG
CATGTTGTGGGTGGAGACGATCCATATTGTACTCGACGACATCTAGTAGATATTGCCGAG
CATCAGCAGCATTATCAAATCCGAGATCGCACAGACCGATCGGATAACGAGCTTGCGTGC
ATGTAACTTTCATAGATACGACGCGGTGTTGTTTGCACATTATATTTTCCTCTTAGATGT
CAAAAACCCTCCCCGATCAGGGGAGGGTATCTCATTGGTACTTCTTCAGCAAAACTGAAA
CTAAGATAAAAGAGCAATCGTAAAGGAACTACATAGAATAGTGTATTTATTTTACTATTT
TTTTACACAACCTCTTTAGATGGGGGTTCTTTATGAATCCAGTCTAAAGTTGTTGGTAAA
CTATTATCTGATCTGAAGGTACACACCGTCCGGAACCGAGAGTGATCAACTACAGATAAT
TGCCCGTTAATCGCAACCTCATATTGCTTACACAACTTCACACACCTTTTCTCTAGATCA
GTTGGTTCGTAACCTTCTTCTCTTGGTTCAGTGTATGGTCCATCCCAAAAGAATATTAAT
CTAAGATCAATCCCAGATTTATACTCCGCTTGGTATGAATATTGATGAACATCAAAATCA
TCTAATTCTTCAACTAGATGTGATGTTATTGTTCTAGGATGTTCATGAGTTAATTCACTG
TATATATGTTTATTTCTAGGGTTTTGTAAATCAACTACGATAAGTTGACTAGTCATTTTA
ACTTTTATCCAGATGTCTATTACGAGTGCGTTCCACTGCTGTAGGTTCGACAGTGGCTCG
CGGTAGATAAGTAATCTCCGGGAAAGTCTTATTAAGAGCATCTTCAATTAGCTCTTTAGC
AATCTCTGCATCGATGTAATGGTTTAGAATAATACCACGATGATACAGGAAGAAGCTGTA
TTCATCATAACCATGGCGAAGACTTACTTTACCACGATACCCACCATAGGTTAGAGTATC
TTCGTTAATTTCATTCCGATAAATATCAAGTTGGATATTATTTTGAAGATGCATTTGAAT
GAAGTCATTACTATCACCGCCGAATAGGGTTGGTCGTTTCCGACCAGTAACAACATCTTG
GATGGAATCCATTAGTCGGTTAATGTCTGTTTCTAATCGTTGGATATCACATTGAGAATT
CATTGCTTCATGGAAACGAGTTACAACTGCATTGAATTCTTCTTCAGTCCAACCACTGGT
ACGTTTTTCATAATCAACAATGATAATACCGAACCAGGCATAACCGTTAGAATGTTTTAC
TTTAACACATAGATGAGGTACATTATCAGCACCTGACTGATGGTTAACAGTGTAATTATT
ATTACGGCTGATTACTAGTTTGATAAAAGGTAGACTAATGATTGACGCCATGGAAATATT
CCTTTTGTATTTTTTAAATTAAAGTAGGAGTTATTATTGAGTTTCAACTCAATACATCAA
TAACTCCAGTATGTTTTCAAAATTAAATACATGTATTTACAGTTACATTGTAACCAGTTA
GTTTTGCCAACAATACAAATTAGCGAACCTGTTCGATTTTTTCAGAAACAATGAAAATAT
GTGCAATACGATCCTCATCAACATACCGATATCGAAATGAGGTATAACTAGATGACACAT
CCGTATCGTTTAAATAGGTGATATTAAAATCATCTACTTCCCTACCATTAGCAGCCGCAA
CGGTATTTAGAATGTTGAGTAGACTCATTTTACAGATTCCTTTTTAGTATATTAATATTT
CAATAGACTATTGATAAAAGCATTAGGGTCATAACGGTTATTTGAAAAAGCCGCCATTAC
ATCACACCCTGGTTGATCTTTAAATCTGATATCTTCAGGAATATCGAAGTCAGGGAACAT
TAGTTTAATATACTCCCTAACCTCTTGATCTCGTAGATATGGAATCTCCACCATGTAATC
AACGCGACCTTTCCTTAGAAGTGCTTTATCAATATTCTCAGGATGGTTTGTAGTTAGAAT
GGTCATAGTTTCATCTAGTGGAACAATGCCATCTAGACCATTTAATAGAGCTGACAAAGT
TAATCCGCTAGCTGGTTGCTCATACGTGATTCCATTCTTCATACACTTCTTTAATTTCCG
CCATCGGCGAATTGTGGCAAATAATGGATCTTTCTCATCGGCCCATTTTGAAACACCCAA
TTCAGCCATAGCATAAGCACTTATCCCAAAACCTTGATCATCACTATTTTCATCAAAGAT
ATAGATGGTGTCATTAAATCCAACTACAGAGTATCCTTCATAGTCATCTTTAAGATCAAC
CCATGAAGATGGATATTTTTCAATTAAATCAATAAAAGAATATTTCCTCTCTAATTCCTT
TATCTCTTTATCTAGTTTTACAGGATCATTTAAAACGCGATCTTTAACTGCCGGGGTATC
ATCGAAGTCCTCAATTAAAAGAATATTACCTTTAGGTAGTGTAGTAAATGCCCGCCTAAG
ACTATCACTAGTCATAGAGGCTAGTGATAGCGCACTAACATTTTTATTAAAATGTGAGGC
AATTGCTTTACTGATCGAGGTTTTACCAGTACCAGGAGGACCTGTTAAAACACAAGTGAA
TTTATAGGGTAGTCCACGATCATCGTACCATTTCCGATCAGAATAGAATTCTTCAATCTT
ATTAAGGAATTCTTCTTTGATCTCTTTACGCAAAATCACCGTGTTGATATCGCGTTTAGT
AACTTCGATTTCATTACCCCAGCGATTACCATCCCAATGTGAGATAGTAAGACCCATTTC
ATTTGGACGCCAGTGGTACGCATCAACTAAATCAATAATAAGTTTACTACTTCTAGATAA
TCCACGGATAGTAATGTCCATCTGGTCTCGACTGGCATTTTGGCTATCCCGGCGTGCTTT
TACAAACCAGAATAACCTACCTTTAAACAGAAAGAAATGTAAGCCAAAACCAACACCAAT
TTTCTGTTTAGTTGCACTATAACTTTGCTCATCAACGTTGGTTGAAAAACGGCGATTAAA
TCCAGCTAATGGTTGTTTAATATACCACTCCATAAATGAGTCAAAGTTTTTCTCATTTAA
ACCATTACCAGTATTAGTGATATGTAAACTAACTGTAAATTGATTTAATGTGAATCTAAC
TATCTTACTTGGTAGACCTCGGATAGTGGTCCATAATACACCACCAATCGCCATACCAAT
CGCACCACCCATCACCATATTCTTTTGAGAGATATCAATAAACATGGCATAATAGCTAAT
GAATGTATCAATCATTGTAAATTCCTATTGAATAAAGAAAAAAATAAAGGGCTCCGAAGA
GCCCTTTACTAGTTAAAGTTTTACAGAATAGGTTTTCTCGAACACTTGGTGAACAAAACG
ATCTGACGCATTATCCCAATTCTCACGACCATCTACACCAAAATAGTGAAATTCTAGTTC
AGTTGGTCCACCAACAAAATCATTTTTAGCAAAGCTAATGACTTTACCATTAGTGAACTT
GACTAATACATCTACATAGAAACCATTGTATGCCCATGCTTTGGCAATTAGTGCAGATTG
CCAGCTATATTGCTTATTGTAAAAATCACGGATAGCACACATTGTCCTAAAATTAAAATT
ACCAGTTCGGATAGAAGTAGAGATAATATTCGTTAGTTTATTAGCTCGATTTTCCTCGCC
AATAAGGTAGATACCTGAAATATCAGTATCCGATACCATGTCTAAAGTAAACGCCATATT
GGATAGTGGTTTAGCCCCATATTCATCGGTGAAGAGAGTTTTAATAACTTCTTTACCTTC
TCGCCGTGCGAACATATTGAGATAACCATCTTCAATATAAGACATATGGCTGGGTTGGAT
ATATTCGCTGGCATCTACTACAATGTTAGTAGCATTTAGATTAACCTTTAGATGATTCAC
TAGGTCATCGATCTTTTTAAAACGTTCGGCTTGTCTAGCGGTATTGAGATCCACGTACAC
CACGGCATCAATATAGTCTAGCGCCATTGGGCTGTTCTGCTTATCAATGTCAGCGTTAAT
ATATACATAATGACCAGCACCAGGACCAAAACAGGTCATGATATTTTCACGTAATTTGAA
TACATCGTTATGATGAATCGGTAATCCAGCACCACCAACAATGTTGAAGCCAATGCGAAT
TGTATTAGAACGACGTGCCATGGTTATATTTCCTATGTTAAATTAATGAATTATTTATTC
AATTACCGTACTACATCTGTGATTAGAATAAGTTCACCATCACTATTGATAACACAATAG
CTTTTTACAATTTCGATGCGCTTACTAATAATCTTATTCTCTAGATCTACTTTTACGATA
TTAACATGAATTACCTTATTCGATTGATCATGCATTTCTTTAAAAATGCATTCGATATTT
TTACCAATGACAGCTTTTAATTTTTCAATGCTGTCAATGGATGAATATTTAAAACTATAT
GCTTCTGCCAGGGTTTTTGCATTACCCACAACAGTAGTTAGGGTACCCCAATAATCATAA
ACATACAGCGCAGTATCACTATTGGGATTATCGATAATTTCAATTATAAAATCCACTTGT
ATCTATCCTATTAGTTTATTGATAGTACACTTGTAATATAGATCTTAAATAAATTATGAT
CTAACCATTTAAAATAGATAAAGACTCTATATGCCAATGGTGTATTAGTTCTTCTTTACT
AATAATATTATTGAAGTAGTTTAATCGATGCGATACCATTAATTTAGAATGCATATCGAC
TTCACTACTTACCTTAATCACCCCAGTCTCTAATAATTGTAGAGAGCCAATATACGCAAA
TAAAGTTGGTTTTAAATACCAGTGTTCCCTATATTGTTTTTGGTCAGGTACATTTCCTAC
AATGTAGTCAGTAGAATTACTTCGGTATGCGTAAATAGTTGATTTAGAACCAAGTAAATT
AAGATCGATATCATAACTCCCGCTTTTAGCTGCAATACATTCAATAACTGATGGGGCCAC
ACATATACGGTTGGTTACACTGTCTTCGCCATCAGCTCTATTACTTGGAATATATGGCCT
TAATAAAACATAATCCCCAAGGTATACTGTCGATATGTGATACATAATTAACCTATTTAA
TTAAAACAGATAACGCTCTGTTACTGAACGATATTGTGACATGATGTTTAATGGATCAAC
ATCCACTGGTGCTTTAGTGACAGTTAAGCGAGCACCTTTTAAAAATTCACCAATATCTAA
TTTCCTTACAGTAACATCATGGATAGTGTTTTTCGTAATCCACTCAGTATCAATGCTATT
GTGAGGATTATTATTCTTAATCCATTTGTCAGAGTCCCTAAATGGAATGGTATAAACATA
CACAACCATTTTTAAAATATCATTTTTATCGATATTATTTTGACAATAGATTGTAATATC
CCTATCAACAGTAGCATATCTATCACTATCAAATAACTTTTCGATAGCGCTACCAATACC
TAAATAAGATGCATCTTTTTCAGATGACGTTGCATATAGGTATTGGTTAGACTCAATACC
ATCCCACATAACAAGTTCACCAGATCTTTTAAATCCTGGCATTAATTCATCTTGTTTATA
AAGAGAACCATGGTAAAGTAATTCTGGTTTATTCATAATAAAATCCACTATGCTTTATAA
GAGTTATGGTTATCAATTAACTTCAAGGTAGCAGTGTGGCCGTATTGAAGTGATTGGTTA
CTGTTTCGATCAGCATATGCTCGATGTTGATTTTGTAGTATTCCGGTTCTTCGTTCAATT
CCCAAATGACAGTCGCACGTAATAGTGCAGGTGCTATGAATCTAACTACCTTACCATGTG
TGTCAAACTCTACATTAAACATTGCATAATGTTCAGTGGTATGATCTTTGTAAAAAGTGA
AAGTACCAGCGTAACTGCGAACCTCTATTTCACCGTGAACACTGGAGTAGACTTCCTGGT
GGAATACTTTACCTTTAAGGCGTGTATTCTCTTCAATGATATTTACCAATTCTTTCAATT
TGTCTTTATCTAGTTGCATTTGTAGGTTCCTATTAATAAATGTATACGATGGATATCTTT
TACGATTTCCGGTGCTTATCCATATCTAGTTTTACATCAGCATATATCTTGAATCTATCA
GATAGACTATCCATATCTACACGCAATGCCCGAATATGCATATGAGTATCTTTTGGTTTA
CCTTCAGACTCCCATACGGCCTTTACGATATCAAATACCACATTGTTCAAATAGTATTCG
ACTTCATGTTCATCAAGCAAAAGAGTGTCATGGAAACCAATACGTGATAAAATGGCGTAA
TCACGCTGATTAACATCTCTCCAACCTTTACGTACTAAGTAACTTTTACAGATTCCATCT
TTTTTACAACTTTTACTAATAATTTCTATAATGAACTTATCATGCCATTTATAATCCATA
ATCATTACTCCTTCATTTAATCAGCTTCATTACTAGAGGAACAGATAGACTTATGCCATT
GGCCATTTTAGCTAGGCTATGATAGACATCGTCTGCTTCTTGATTATTGGTACAAGGTAT
AGAAATGGCATCACCATAGTAAGGACTGTAGTTAATACGGTCGCCATACACACCACTCAC
AAAACTATAATCTAATCCATCCGTAGTAGTCATAATAACTACAATATGATCATCCCCAAC
TATACCGGATTCTAAATCCTCACATAGTTCCAGTTTTTGTTGCACACCTCGTTGACAGAT
CCATACCTCACGGTATTGATGATGGGGATATGGATTACATGCTACATCATTTTCACTAAC
GAAGTACATAGTCCCCCAGTTGTAAGACTGGAATTGGGTTGTTTGTTTGTGGGCTTCCCA
TACCTCCTCCATACAAAAGGTATGTTTGTTAACCCATAGACCATCGTTATTAAGACTATC
AACGATCCCATCACGGACACGTACTACACGGAGTGTTTTAGTCATCTACGTAACCCTCAT
CAGATACATAAGTGCGTTCAGGGCTGAACACATATTTGATGTTATTAATTACAGCTGTGA
TATAAACCTGATATTCTTTTGAAATAGATTCCATCAGTTCAATAGATACGGAATTTGAAC
TCTGAATAAGTTTATTTACTTTCTTTAGTTCATCAAATTCTAACCGACCGTAGATTACAC
CAACATCTCTGAAACTGATAACTGGATAGTGTTTCCAAATATAACAATTCAGTTTACCAA
GATGAATTACTTGAATTGTTTCATCGCACCATTGTTTCTTTTTCTTATTGTAGTATTCAA
CACGACGTTCGATGAAGACGCGAGTACCAGAAACAGAGGTGTACGCATTCAGTTCATAAC
CATACTTATGGTCAACGTTATTTGGATCTTGGCGCCATTTTGGAATGAATGCTTTCATTT
GTATTTTCCTTTTTACGAAATAGAAGCCCTCCCGAAGGAGGGCAATTATACCATTATTAA
ATTACGAGTCTATATACTTCTTTTAATTTTTCACCAGGGGTAGCTAGATAATCTACATTA
TATTTAATAGCAATTACACCCACAAGTTCTTTTCTCAGTAGCTGTTCTACCTGATCGCAT
AATTTATCAATAGCTCCATCTTCTACATCCTCGTATAATGGATATTGACTATAAATATTA
TCAGGTGTAAGTACAGACAGTTTCAATTCCCCATCTTTACCAATGACTGGGTGATATGTC
TGTTTAAGACCTTTACAATGATAGTGATAGGTAATGCGCTGTTGTTTAGTGGTCATGTAA
AGATACCTTAAATAAACCGTTGGGTAGTTCCGTCTTTAAAGACAGTTAGTTGATTAACAT
CTTTCAATGAATTATCTGCAATATTCTTAGCCAATAACAATAGATCATCGTGATATGTTT
TAATGAGAACTTTAAGCCTCTCCCTTTGTTTATATCGGAAACCTTTATTAATCACAGTAA
CTCCATCTGGATTATTTTTATAATTCAGTACAGTGATTGGTGTATTCGGTACAAAGACGG
CTAGTTCGTCATCGAATACAATGTAAACCATCACTTTATAATTATTAACCGATTCTTGGA
ATTGTTGTGTGATTACATTATAGTCTTTTGTGCGATGGCTAAGAAACAGCTCACTATTAG
CTCGGCAGAAACTATGTCGCGCTTTTTTATCAGCACTCATAACAGCGATACCACGGATAA
TGAATTTATTATCTAAGGTATATTTTCTTTGAGACATCGCATAGTCGACTACATCAACAT
CGCCGCCAAATAGCACGGCATTTTCACCGTATCCACAAATAAGAGCCATTTATAATTACC
TGTTTAAAAATTATATTCAATAAATTGAAGAGTGAATAGTTCACTACATTCCCATGTACG
ACTTGCTAATTTATATTTACCTTTAACAGGTAAGTCAAAATAACGTAATGCTTTTTCTAC
TGAACCAGCCTCATGATTAACCAATAGAACATTCCTAGCCTTCAGAATATTTGAACAATT
TACAAGTTCATAATCATCTCGCTTAGGTGTCAACATAGCTACATTAGTGCCTGGTTTTCT
AGCATACCAGAGCACCTCATGACCCTCTGGATTAATAATAGAGATATATAGCTGACGTTC
AGCGGGTAACCACAAGTTACGTGGATCATACTCTCCATTAACATCAAAGAATTTAAATAT
CATAGAGTTGGAACCGGTTTTGATGGATCAGCAATTAGTGCAGATTCTTCTACATAAACC
ATTGCGAAGTTTTCACCTTCTTTACGAATACCAGTAGAAGGTGTTTTCGATGGATCATAA
ACGATAATTTGGCACTGCTGTCCAGCTGTTAAATAAGTTGGGCCAGTTAATGCATTGGGT
TTTGGATGAGGAATATCAATTTTAGTAATACAATATTGACCAGTACGAAACTCACGCATT
ATATTAACCTCTTAATGAATAACTGTTGCAGAAGGACGACATTTCATAGTTTTAAAAATG
TCAGTAGGATAGATATTACCAGCTTGTCCGAAAGGTTTATCGGCTAGAAGAAACTGCGGA
CAAACTGTTTGTAGATAACCACTATCAGTGGGTAGGAATCCTTCAATATCACTGATGAGA
ATTTGCCAGATGGGCAATTGGTTATAATAGTCATCCATATTGTAGTATGCGTGGAGTTTA
CCACCGAACCCATATACAAACTGATGAGTATCAATTAGACGAATACCAAATTTAGGTTGT
CGTCCCATCCTTTGATCTTCTTCAAATGCGCTCAGTAAATCTGCAATCTCTACGCTACCC
GTGATCTGTTTCACTTCTTTTAATTTTTCAAAAATGGCGTAAAAGGTATTTACTGCCGAT
TCCTTAGTGATGCATGCAGTGATCACCAGTTCAGGAAAGAGTGGATCTTCTTTATTACAC
CCACACGTGTAATAAACAACATTGGGACCAATATCACCTTCTCGTTCAAGCACGGCAAAA
GGCTTTTTGGATAGTTTCATTTGCTTTTCAATTAACTGTCTGCTATTCTCACGAATAGAT
GCATATTCGTGACTGGCCTTAAAAATAGGCGCTTTGTAATCTATCTCTTTTGGTTCTACT
AGATCACTTGCCGATTTGATAACTAATGGGTTACTCATTGTATATTTCCTTTTCTCATAT
AAGAAAAGGGAGGTATTACCCTCCCTAATTAAGCATTAGATATTTAGGTTCAGTGGTGGC
TCGGATCTTAGCAATAAGTGCTTTCTGATCACTAACCTTACTAATGGGGTATTCTAATAA
ACGTAAATCCATGTCCCGATGTACTAAACCCAACATTGCCTTATTTTGATGACTATTATT
ACGTAATGCAGCGATATGTTTATCAACTTCATTACTAACAGTCTTTGAAAGCCCTACTAA
ATACTTCCCAGTTGCAATGTGTTCAATAACATAAACACCTGCTCCAACTATTTCGCTTCT
TTTCCCACGACCGTGCGAAGTACTTGACCAACTATAACACTTATCATCAACTATAAACAT
GGTTAATTCCCGTCCTATAACTATCTAATTGGACGGTCAAGTATTAGCTACCAGTCCATG
AATACTTATCATAAACTAACCACATTGAATGGATCTTATTGATGATATTGAAAATAGGTT
CTTGGACATCTTCAAAATCACCTAGATCAGCTGCCAGATTGGCCATATATTGGAATAGCC
GCTCAGTTGATACATCCACCAGATTGGCAAATGGAATACTGGCATATGGGATTTGCTTTA
ACATACCAAACTTATAAATTTTAGCAAACCATGCATCCAACATTGCAACATCTGCTTTAA
ACATCCAATAAGTTTCAGCTGGATATGTGCCGTCATGGATAGAGCAGAACAAATCACCAG
TAATCGTTTCAACAAGCATTTCTACTTCAGCAGAATTAATTTCGATCATCTCAACCCACC
TTTAGTTTACTGCAATATAGTCTAAACTTAAGAATATCCTCTACTAAGTTAGACCATAGA
GTATACCTATTTCGTGATGCATTTACTTCTTCAATCTTAGCATTTATATTTTGGCGAAGA
ACTTTGTAATGCTGATACGCATTAGTCGCATGTTTAGAGAATAAGATAATAATATCATCA
GGACTTTTATTTAGCACATCAGCTAGTGAATCATCATCATATATTTCAATAAGCGCATTA
ACGATACGATGATGAAGATGGGTTAGTAATTGGTAAATATGATATTCGCTACCAATATAA
TTACCTTTTATAAAAGCTCTTTTACTGTAGAGGATCTGTGCAGTATCTCTTACGCATTCA
AGTTTCATTGTGCATTAACCTGATGTAGATGAACATAAAATCCATTACTAGGACCGCTAT
TAGATGGATATACTAACAGATGGTCTCCATTAACAGACTCGATTATAACTACAGTGCCAG
CTTTGCATAGTGTAAGATCATAGCCGCATTCACCGGTGCTATTAATAATATCTGTATTGA
CTACAACTTTGTCACCGACTTTAAATGAATTTGACATATATCACCTCAATTATAGGCGTG
AAGAATATCATCCATATCTGTACATTGTACAGTTTCAATTTGATTATAATTGTCGGCATT
TAAAACTCTAAAGCCGCCTTCGGGATTACCTTCATCAACGCCTAATACAGTTAGGTCATC
TCCTTCATCGGCAAACTTCCATTCTAGATAACCATTCTTTTTACAAGTACGCATGATCCA
AATATCCTGTGTTGCTTGTAGAATCATACCAACTTGAACTTTATTCTTAGCGATAAGACT
AATACCATTAGCTAGCGCGCTTGCTAGACAGCTCACTGTTGAACTCATTTCTTTTTACCT
CTTTACAACTCTTCTCTTCAGTCAGCCATGACTTGTTAAAGATGCTATTTATGATAGGAT
TGAATTTCGGTGATTCTTTCTTTTTCTGATCGGTATCCATCGATATCACCTATATTGATA
ATTTTATTAATACCAATTAGCGTACTACCTAAAACCGCTATAACTAAAAACAATGCTACA
TATTTCATTCCTTCCATTTGTGATACCTCAGATAATATTTATTTTATTTGGCATGGTTGT
TATATAACTTTAAATTTTATTATAATAAACTATTACCCCGTATTAAGGTAATAGTTTATT
ATATTTATTATAGTTATCGATAACCCATTGGCATGACATATGTTTAGGAGTATCACCTAG
TAATTTGGTGTACGTTAGAAAATATATTACTTGGGCTTTGTTAAAGTCCAATCCAGCTTC
TTCCCAGATATAACGATACTGTTCAGTTTTATCCCCATGTGCAGCAACCAGACCAGCGCA
TGCATCAATGTATCGATCCTCACCAGAGAAAACAGCACTTATGAATTTATCAGTATTATC
CAACATATACTGAGTCGATTTGATTACCTTATCATTCATTTTCATTTGAATATACCTGTT
TATATAAAAGGATATAAAAGCCTCCCCGAAGGGAGGCCATTATGACGTTACATTGTGAAA
CGAATTGACACACCACAAATAGGACTATTGTCATCTGTCCAAATTTTTGCAGTACCATCA
TTTTTAATAGTTAAACAATAACCCCAACGGAGATTATTTATTTTAGTGGTATAACCACTT
TTGAATACAGCCATGTCTTTATAAAGCTGAGGGATAGTTGGCACTACCTTTGACTGCATT
TTACCAGATAAATCACGATAGTAGAGTTTATCTACAATCACATTATCAAATTTTTTATAA
TCACCAAAGACTTCAGATAGATTAAACTCTACCCCATCTGCTTTCAATGGAGCTGTCATA
AAACTACCTTGCTCAGTTGTAACCATACTGATAGCAACATCATGGTCTAATAGAAATCGA
GTTTCACCATTAGTTTTCTTATATACTGGGAAACTTCCTAGCGGTTGTAGAACACTCATT
ATTAACTCCTGATTGAAGGTCTATAAAATTGACCACTATAGACCCATATAGCATTCCACA
CCAATCTTTTTTATAACCATATCAGACTCACTATCAACATATTCATTATCATGGTAATAC
TCATCTGAATAGCCAATAAGTTTTTGTTGATTGTGATAATTTAAAAAGAAAATAACACGC
ATTACAATACATGCATACCCAGGATGATCAAGATACAGTTCTTCTTTATCTAGATCAAAG
AAAAGAGTTTTTATTTGAACTGGTTCATTTGTGAAGATTAACTTTATTGGATTATCTAAC
TGAACTACCTTAGGGTCACTATCGAAAACCGTAGTATCATGTTTACCTAAGTAATACGTA
ATTGATTTAACATAGTAAGTTATATCCTCGGTTTTCTCTAACCGGGTTATACAATAGCCG
GTTATGACTGAAAGAACAAACAATGATAAAATAACTAATATTATCTCAGTCACATTGACC
ACCCATATAAAGTGTTGTTATATCGATACACTGCGAAACAGAATGCTTACATGGAATAAA
TATTTCATCGATGGTTAAATCATCATCAATAGGTTGAATACTTTCTAATCTACCAAATCC
ATCACAGTTAAATTTAAGTGCTGGATAACCTTCAATACCCATGCTAATCCAACTATTATT
ATGTTTAGTTACATGGTTGAATTTAAAATTACGCATCTTTCCTAAAAATTCAATTGTCCA
TGTAGCATCCTTCTGCTTCTTCAAATGGTAATATTTATTCATTAGTATTCACCATTATTA
AGATTAGTTTCATTCTATTAAATAGATAGGGGGTCCAAAGACCCCCATTTTCATAATTAA
TTAGAATGCAGAAATGGTCAACTCATTGCACTTAACGGCAGCGAGTTTTTCCCAATTTTC
TTCATGTTCCATCAAGCATTCAATTACAACATGCATGGACCATGACTCGCCATCAATATT
TGCAAATACAAAGAAACCATCATCAAGCTCAGTAATGCCACTAATCAGATTATTTTTGAT
CAGGTAGACCAATTGATCATTAACGATTTTAACTTCATGATCAATGAGATGGCTGGCATA
TTTGCTAGTGATATCAAACACTTCTTTTGATGAAAGTGATCCATCGCGTTTGACGGCAGT
TAGGCTCCAATTAACCAAAGACGGTGAACACCAATGAAGACCATGCACATTCATCAAAAC
TGGATGTTTGTTATCCATAGCTTTTACAGAATGAGCCCAGTACTTTTCATCAATATACAT
TTATATTTCCTTTTTTTCCAGATTGATTATTTTATTACAATGCCTTCAGCATTGCGTACT
GCTTCTTTAGCAAATTGAACATCTCCAAATACATGTTCAATAAACTTTGATGCATCTGAC
CCAGCTACATCGCTTGATAGTAGTTTCACAGTCTTGGTAATCTTACCAGGATAATAGAGG
ATGATATCCCATCCATCACTTTCTTTAACAGAGCGAATTTTTAGAATAGGCTCTTCTTTA
CCAAGGTGTTCAAGACTAACACCACTTTTGACAGCGGCTTCATATACTACATCAGAGATA
GACATATTAGTTACCTTCTTAAAAGAAAGCGGTCATGCGACCGCATCTTTAATCAGCGAG
ATAATTCGCGCTGATGGTTCGCGCCAAAATTCAATACGTACATTATTGAACTGGCTATCG
TGATGCATAAAAGCTGACGATTGCTTTGCTGCATCGTGATTCATAATAGAACGTAGCTGG
ATAATATTTTTGTCTTTGAGTCGGATACCATCCAGATCAATGACTTTCAGTCCAGAGTTC
ATAGTTTTTACAGTTACATGTTGCATTTACAATTTCCTTTTAGTTGGATTAAAAATCTGT
TAAACTTCTTTTATAAGTTATTTCGGTTTTGCGATAGCTTTAATGAGTTCCTGTTCAAGT
TGCACGAGTTCATCATCTGCGATGTCGCGTGAAACACACATACACCGTCCTGGTCGTTTA
TACCAAGTAATGCAGACATCACGCCAAATAAGCGCACCTTCATCGATATCATTGAGCCTG
AAAGTCTTTAGCTCGTCAGTGTTAAGAGAAGGACCACCAGGATAAGGATAAAATCTTTCG
TAATCAATTCCACTATATTTCATCACTAGGGCATATAGTGGTGGTTGAACCATTTTCCAA
TATGAAGCTAATTTAATATCCTCACTTGGTACAGATGCCCAATACTCTTTTTCTGCTTCA
TCCATTTCTTCTGGTTTAGGAAGTCCACCAATCAGACAATATGCATGTTCAGCAACTTCA
ACTAAGCGTGTATTTTTCATAGCTTAATCCTAATCTTTGTAATACCGCAATCGTGCTCAC
CGAACTCGACTTTTCTCCAGCCCGCTTCACGCAATCGTGCTTTAACAACCTTCATTACAG
ATGGAGCAATTTCATCCTTATGTTCGACAACAATTTCATAACTGCGAATAAAATGGAAGT
TGTTGAAACCAGGTAGGTCTTCAAGCATCTTTCTCCGAATACCCGCACAGACTTCATTGG
CTTGCCGTTCAACCAACATTGCATGAATTTCATTTTTGGTCGGAATAGAGCAATTGCCAG
TTTCTATTTGAAGAGTTTTGATTGCATTTTCCAAAAGAGATTTTGAAATACCAAGAGTCA
TCGTATCAGTTCCCCGTGAGTTAAATAAATTTTAATACGCGTTTTGATGGTTTATTATTT
ACCAATAGATACAATTACCTCTTTTAATTTACCATGGTCATTAATTGCCATCTGACAAGT
GGGGCACGGATACGCATCCAATGGCCTGCCATCAGCACCGACCCGCGCAACATAGATCCT
ATATCCTTTTCCTTTGGAGAGGATGATAGAACGCAACTCACTATGCATCTCACAACGTAG
ATCAGACAATCCTGCTTTTAGAGAATAACGTTTCTGAACAGGATGTGTTTTACCATACAT
ATTTTGGCTTTCACTAATAATCCTGCCACGCTTATCAGTAATGACTGAATAAACACGTTG
ACGATGTTTTACATAAGGGATCTCGCGACACTTTTCCAAAGCATAATCCAACATAATTGA
TTTACCTATTTGAAATTTAGATAGTTTTATCACATTTGTAATATACTGTTTAAAATGCTT
TAAATACAAAAAAATAAAATATAAGCCCTCCCGCAATGGGAGGGCAATATAATAACTATT
ACAATTTATTCGCCATTTTACCTGGTACTATAACCCAGTGTAAATTACATTTAATGTGTT
TATTTTTAAGAGACATTCCTGAGATACACACATCCTCTTTGGTTATGCTAATTTTAAATA
AAGCTTCCCATTTATTAGCTGTGATGATTTCATCAACAGTGTGATGGAATTTAAGTGTGT
TATCTAATACTGAAATATAAAAGTCTTTATTGGGTTGACCTTTAGTTAAATCAATTATTA
ACCTATTATTTAATTTAGGTGAAAAAGTTGTTAATGTACCATCTTCATTATTTTCAGATA
TATAAGAGCAACCAGTAACAATGAAGACATTATCATTTACACTATTGGCATAAATTATTT
TTGGGTGTTTAAAAATATTAATTAGATAAACAATAATTGCTGACAGGATTAGTGCAATTA
CTATATCAGCCATTATTAACCCCTATTAACAAAGATAAAATAATCTCGTAGTTCACCAGC
TAAATCTAATGGTTGATAATTATCACTACCATCATCATAAGGGATATAATCAGCACCTGC
TTCTACAGCCGCTAAATATTCTTCCTCACCAACTGCTGGAATTAAATGAAACGCTGGGCA
ATCGTCATTATTGCCATCTAGTATATCTAGCATGCCTGTAATGACACTATCTGCTACAGC
CCTGTCCCGATCAGCTGGCTCTGTAAATTGATCAAGTTTAGAGTTATTGATAGCATTATC
ACGCATATTGACAACACTACTAATAAATAGTTCTTTAACTTGTTCCTGTGTTACAGGTTT
AACATTACCATCGCCTTCAAAATAGATCATAATTACATACCTTGTTATCAGTAGCTTATA
AATTTATTAGTTCATTAACGCATTATTCGACTGATATCATCAGCTATTTGTGATTGTATC
TTTTCCGCTTTTTCTTTAGCTTTTTCTACTAACATAGCAAGTTGCTCTTGCGTAAAATTA
CCCGAGTAATACTTAACAGGTTTCTCACCTGCTAAACTAAGGAAATGGTTCAATAGTGCA
TGTGGTCCTTTGAAAGGTTCCGTAAATGTCTTAGAAACTTGATAACGTAGATTACCAGAA
ATATCAACTGCATCACACTTTGCACGGATATTATCTTTTAAATTTTCAACTGGGTTTACT
GGCCAATAATTCTCACCAATACTTATTAGGTAGTCAATATCCTCTTGAGTAACATTAGGT
ATAAGTTTGAATCCGGGTATATTTGGATCATTGCCATCTAGGCCAGCTGCGATGCTATAT
ATAAATCCATCAAGCATATCTCTGATAGATAGTGCCTTATCTGTATTTTGAGTATCGGTA
TACTGTTTATGCCAGTAATTACGTGTGCTTTGAAGCCAATTTACAAAGAGAGAAGTTTGT
TCCTCTTTTGTATATTCTCTCGGATCATCTACTTTCTCCCATTCTTTTTGTTTATTTATC
TTAGGAGTCTTAGTTTTCTTTACAGGTTGTTCAATCGGTGAATCTGAAACCTTTGTTTCA
AATCCTATACCAACTATAGTTTTATCGGTTGTATTGATCATAGAACATGAAACCTTTGTA
TATAGTTTGGTTTTATGCGTTTAGTAAATTGCGCTGAATGCTCAATGTAATAAATGCATT
TATCATTAAGTGAATACACTGTATAATAGTTTTTAGCACATATATGTCGTATGCCAATAG
TAACACATATTTCCCAACCGATCTTTGTAAATACATAACCTAACTGATTAGGCTCTATTT
CTAAACATCTTTTGTCGAAGTTAACCATTGTGGCTTCCTCGGAATAGTTCGCAAGTACTG
AACAATAAGTCGCGCGAATTCATCCATATCGTCTTCAACTAGAATTAACTCATCCAATGT
ATACCAGCCATCATCAGTTGTTTCAATTAGATTAGAATCATCTTTGATTTCTCTATCTAA
GATGGCGTATAATCTAGTTGGTCCAAAATCAGGCTCTTCTATAACCCCAAGATGTATCAA
AGAATTTCTATTATTAGTATCATCTGTGATAGTTGGCTGAACACTAATATTGGAAGTTTC
CTCTGGGAAGTTATAACTATCTAAAATAGCAAAATCAGTTAATTGTCTAATAGCCTTTCT
AGTGAAATCATAAACCTTCACTATGGGATGAATCACATAGGTTAGTTTATCATTAACTAA
ACGTTGCCTTAAATTAATAAATGCGCAGTTATTCATCGACATACTCATGTACCATTTTAA
TAGTTTTCAAAAATACTTCTTGATTATTACTATCATCTGCTTTTAGAATAACAGGATGAC
ACATAAATTCATTTTCCAATGATACTCGATAATATTCATTAATTGTCTGAATGAAATCAA
GCGGGGGATAAATAATAATATCCTTATCTGTTTCGGCGTAATTAACTACATTTGGTTCAT
GTTTAAATTTGGATTTATTTAATTTATAAACAAAGTATTTTATATCATTTTTATCTACTT
GTGATACAAATACAATTCGTTCATACATTGATAAATCAATATCTTTACCAATATTGATAG
ACAGTGGCATACCAATAAATGAGCGTTTAATATTTTCATTCATTTTTTAATTACCTATTT
AATAGTTTAATGTTGTATTAGTGTAATATAGATTTCAAAAAAAATTTAATGTAAAAGCCC
TCTCCTTTGGGAGAGGGTTTATGACTTTATTTATATTTTTTCTTATGAGTGTGATAAATA
TAGCTAAATAGCATACCGATTGACATTGATACAGCTACCATACCCACATAGAAAAGTTCA
TTACTAAGTTGCATTAGATTTCCTCAAATAGTTTAATTTATCCCACCTAGTTTTAACCGT
CCAGTCGGTTAAAAAGAGGACCCGAAGGTCCTCTAATTATTTCTTATAACTCTCAGGGCA
TCCCATTTTGAGTTTGCCATCCTTTGGGTCTTCAGCGAATAATACTTCATCTGGACACTT
CTTAACAGGAGTGTCCTTCGCCTTATCTTCCCATTCTACAACAGGCATTTCTAATTTCTT
ACGAAATTGATACGTATCATAAAGAACATACGTAATCATTCCCATAAGAGAGATTACCCA
TACCCATGCAACAATTAGGATGATTCTAGTTATCATACAACTCCTCGCCGACTATATTGA
AACACCCATTACTGAGTTTTTCAGTCAGCATCTCACCAAGATCACTTCCCTCGCTCTTTG
CCAAAAGCTTAAGTGCGGGATCAGTGAGATTTTGGTATATTACATCGACATTATCCAATG
CGATATTAATAGCGAGTGCCTTATCACTCTGGTTTTTATCTATGGCAATGGATAGTTCAT
CTTGCAGTATTGCATACATCTCCAATGGCTCACCGCCATTAGTGTAGAATTGAACCATAT
TCTTTGCCAAGCTGTGCAGCACGTAGCAAGATGACACGGTTACGGGTTGTGCATTAACCG
ACATTGCTACCATAGTGATAATGAGCGAGAAGACGTATTTCATCCTATTTTCCTTTTGAA
AGTATTCGATATGAATTAAATGAGTTCAACAATTACCATGTGAGTGAATTTCTCACCAAT
ATGCTTCATGCAGTAATTGCCATAATACCCGCTGATAAAAGCTGCCAGATTAGGAGTAGT
CACATTGTAACCTTGACTACGGATATAATCATGGATCTCTTTACTGTTAAGGTCATTATT
GATTTTATTAAGACTATTGAATACGAATGTTAACGCAATCCAATTATTGGTATCCAACTC
ATCTTTGAGTTTAGCAGAGTTGTTGAGCATAAGCACACCAACTTCATCTGCGCTATGACC
TTCACTCATTCGAAATACAGCTTGTTTAGTAATGGTTTCAACCATTGCACACGACTGCAC
AGTGAGTTGAGCATCTTTTTTACCAATCTTGATTTCAGCCATTGCGGTATTGAACATGAT
ACCGGTAATGGCAATGAGACCAGCGAAAGCTGCTGCAATTACATTTTTCATTTTAAATCC
TTCTTCATTTATTTAATGGTAAGAATAGTAATTTTTTCAGGATTTACTGGAACTTTAATT
TTAGATTTATGTTGGAGAAACTCAACAACCACCATAGACTTATCCATTTTATACACTTCA
CATACTTTGACAAGCCTATCTACTACGCAACCAATTTTACCGGCATTTTGAGAAAAGAAA
GATTTTGCTTTTCTAATAGCCTCGCGTTTCTCAAGCATTTCCATATGCATATTTTCAATA
TGTTTATCGCGTTGAACACGATTAAACTTACGCATATGTCGTTCGATCTTACTAATTTCA
GAAGAGTGTAATGAATAAGCAGCAGCTTCTTTCAGTTTCTCATATACTTCCACTGCCATT
ACCACGTGATCATCTTCAAAATTTCTTTTCATATCTATGCCTTTTTTACTGTTTGATTAA
ATTTGACACTGCTTCTTGTTTACATTGAGTATTCATCCATAATGCAATGTCTTTGGGTTC
TTGTCCAGTTTCCACCATATCGATAATGATACGGGTATTACGTTGTGCTTTCTCACGGGC
ATTTAACATATCAGGATCATTAATAATGAATTCCCTACCCATAACTGGATCTAATCCAAT
ATAAGCTTTAGATGCAATAGTTCTCACCATTGCTTCATATTGGTCACATCCACGTTCAGT
TAATACAAGGTTATCTGCATTGGCACTACCGCCAAATATCAAACCTAGGAATCCATAGAA
AAAAGTGTGACGTATTGTCTTCCAACAAATACGCATCTCTTCATTTTTTGTATAAAGCTT
TTCCCAATCAAACATTAAATAGAAACTTTCTTTTTGGATAGAAGGGTGTAACCAAATAGG
TTTAATACTTACTTCAAGAAGTTTACTATTTGGATTCTTACCGATAAAGATAACTTCAAC
AATGGCTCGTGCTAAAGCATCTTTTTCTTTACCATTCCATCCAAATTTTATTTCATCAGC
CGTAATGTAAAGACGTATTCCATCACCCACGTATTTGGGTAACCAAACAGCTTTACCTAG
TTGCACTGGAACATCATTAGGGATTCCCCCAACCATCCAGGTGGCCTTGGTGAAATGAAT
AGCAACATCATCATTCAACCATTTTAGAATAGCTGGCCTTACCCAACTATCTACCAAATT
ACCACATGTCAATGCAGCTAGCATAGCCATGAGCATAATGACAATATAGATACTAATCAT
AGTCTACTCCAAGTTAAATTTAGTACCAATTGCTTCAACACAATTTTCAGCTAACCTATC
TAGGATAGCTCCATTGGTAAATCCTTTTCGTTTATTTACTCGAATATCAACTAAGTTTAA
CTTGGCATATACCGCACCTGCATAGAACTCCAAGTCATCCATATCCTTACGAGAGATCAT
GTCTAACTGCATCATTATCTCAAATTGGGATAGGTTCTTATCATAAAGCTCAGTTGCTTT
TTGTGAAAGATTTACTGCCACATTACATGATTTCTTAGTAATACCCACTTCTACAGCGTG
GGCAGGCTGACATGCAATGGACAGGATTGGCAGGAAAAGGAATAATAGCCAATTCTTAAG
CATGTTGTACCTCCATTATTTTAGAGTAAGTGGTTTTGCTTCAAACGCACCTGCCGCTAC
ACAACGCTGGAAATAACCATTAATGATGAATTTCTCAGGAGTGGTCTTATAGACTTTAGC
AGCATTTGTTGCACGTGGCAAACTTGCCTCAATTGCCATTTTATCTACCTTAGACCATAG
GTAAAATAGACCAGTGAGTTCTTCAACTTTAGACTGGTTGATCTTTAGATCTTTAGTGTT
ATTAAGGATATAATGAATCCTTTCATAAGTCTTCATTTGCACTTCAGTTACAGATGCACC
TTTTACAAACAATTCATCTGCCTGTTTTACAAACTCGATGCATGAATCACGATTAGTGTA
CATGACAGCCGAGTTAGCAGTGATAGTTACAATAGACAACATAAATGCAATTACATATTT
CATAATGAATTACCTCAAAATTAATTTTATTAACTATTGGTTTCACCACGAGTATTGAAT
TCTAAATCCAATACAACTTTAGTAAATTCTTCCAGTGCACCACCCTCGTAGATGAGTGGG
TCACTGTCACCCCGGTACATGCTACCTACCGGTACTTGACCACGAAACACCATCAGCATA
TCGTTTTTTACTATGACATGATGTTCATCCCCGACAGCAATGTCTTGGATGTGTCTCCAG
CGTTCACGCAGTAAGCTGCAAGCGCACCATACGGATTTCAAACGTTCAGTCATTGTATTG
CCCTCTTGGTTATTAAATTCATATTTGTAATATAGCTTTTTAAATAGTTTAAATACAAAA
AAAGATAAATAAAACCCTCCCCGAAGGGAGGGTGTATATTAAGTAATTATTTTAACATAC
AATTTCATTATCAGAAAATAGTTTATTATATTCTTTTAGTGCATCCCCACCAACCATAGT
ACACCTGCCATAGAGAACAGATATAGCCAGATCTTTAATAGCGGGGTGACAATTATTTAA
CTTACTGATTAGTTGAGCTTTCTCAGTCAGATACAGTTCAGCAAAATCAGGCTTATCATT
AACAATCGACCACATGTTTGAAATGGAATCTAGTAACTTAAGAGATTGACCACTCCATGA
AATGCGAGAAAGTCTTTCATGTTGTAAACTAATCCTAGTTTTACGATTACCTTCGTAAGT
TACATTTGTTAGTTCAGTTACTAAATTAACAACAGGGATTGAGAATAGTTTAGTTAGATC
AGAAGCAGTTACAGCAGTATCCTCAATTACATCATGTAACCATGCTGCTTTATAAGCAGC
GTGAGATAGGTCGAATAATTGCGCCATCTCAGCTACTTGTAATAAATGATGCCAGTAAGG
TTTTCCTGAACCATCTCGCAATTGACCAACAGCAGTATGTGCCGCAATGGCATATGATGC
AGCCATTGCATTACCCGCATTGGAATATTTGCCATTACCTAGATGGTTAGTTAAGAGAAT
ATTCTCTTTCCCTCCTTCTAGAATATCGATAATTTCATTCATTAGCATAGCTATATTCCT
ATTTAAATTATTGGAAACCTAATATAGGTCTTCACTAAATAAATATTTAGTTACTAATCT
ATTTTCTAATTCAGCTCGATATAACTGATTATTATTCTTAGCGATAATTACTGCTTCTTC
CCTAGAGTGGAAGTTTTCATACTGATCAATAAAACCTTGTTCATGATCATCTCCTGCATA
TTCATGCAATACCTTAAGACCACCATACATTAGCTGTTGTTGAGTCATTATGATTGATGA
ATGCCTCGCTGCAATGAAATAAACATCCTTCCATCTATTGCAGGCGGCAACTATCCTTAA
TTTCACATCGGGATGTTGTTCCCACCACCATAGACCAGTTTCCTCATTCCAATACCTTTC
TTTCATTTGAGTTTTTCTAAACTCACCTTGTATGTCTAAAAGTCTAAATGCCTCTTCTTT
AGACATAGCTTGTAGAATAGGATACTTATCCATTTAATACAGATACCTAATTGCGAAATA
GAAAGAGCCCCGAAGGGCTCTTATTATACAAGTAGCTTTTCAATAGTGAAGCTTGTCCAG
ACCATTGCCCTATAACCAACACTCTCTTCAGTATAAGTGATATCATCAAACTTATTAGGT
GGCATGAAACCACGTTGTGTACTAGCATCAGTCACAACAAGATTAGAATCTTTTAAAGTT
TCATTTAGAGCCTTAAGTTTATCCTTACTAATTGATCCCTTAACATGGAACTTGAAATAA
GCATATCCTTTATGATTGATACGACAACTGTCAAGAACAGTTTGTTTATCATTCATTTGG
CTCATCAGATCACTAATAACTGCATCATCTTCAGGAGTATCGTCATTGACAATATTGATT
AATTCAATATAAGTTGGCATTCTTTTTGTTTCCTTAATTAGATTGACTACATAAGATAAT
TTTTTAATTAATATTCTATTAAATTTTTAATAGGAGCCCACTGTGATTAAATGAGATAAT
ACAGTTATGTCCCATCATGGCAATATGATAAGATATTATATTATCACGTGTATCATTTGA
AATCTTGTTCACCTTAAATTCATTCATGATCGCATTATGAATGGCTAAGTCTTTTTCTCG
ATACTGCTTTGAGAAAAACATAATATCATTATGATTAATATTGATAGTATCCATTGTATT
AAGAAAATCTTCAACATCAAACTTTTTAATAGTTTCAGATTTTCCAACACAACGCTCGGT
TTTATCAATTAATGAAAAACCAATAGTCCTAATTGGTTCATCCTTATTATGGAAGCTATA
ATTGGTAATACTCATACCTACCAATAGTCCATTTTTATCTACATTAACTACTTTAGAATC
AGTGTACTTATAAATATCATTGATTGCATAACCAGCTGAATATAATACATCTTTAAAATG
ACTAGTTACTTTATCAATAATAAAGTTTTGATAATCTGATTGATATAGTTTATCACTATA
TTCAAGCTTTTTGATAATATCACTTAGTTCATGATACACATTGGTAAAACTATTCGAAAA
ATAATTATCTTTTATTCCTAACCTAGTTAACAGATCTAGCTGGATTTCTTTTTTAGATAG
CTTCGTAGAATTACTAAGTTTTTCAATGACGGATGTAATCGTACGTAGATCAATTGCTAG
TGAAAGTGATTTAAAGATATTATTAATTGGTGTATCAGATAGCTTTGAAATAGCTATTAG
TAGATTATCTAATTCATCATTAGTAATTTTCAGTTCTTTCACAATGTCATTATATTCAAT
TAGTTTACCATCAATAATATTACAGATTAGAAGTTTATGTAATTCTTTAATATTGGTATT
AACTTGTAGATTATTCATTTTTTTACCTTTTAGTTAATTTAAAATTTATTTACAGTTCTA
AGAAAATATTCAGATGGATCGTCGAAGAAGACAATACTATCAACAACACTATTTTGTCGA
GTACACCTTACTTTGATAAGATTATATTCTCTGAATTTATTAAGACCCCAAGCTTCTTTA
GCACGATGGCGTAATCCACTACCAACAATAGCCCCATACCTAGTTTGTTCAACAAATAAA
TAAACATCCCAGCCATGATGATTATCATGGTATTTTGCGTAGTATTTATTGTCTTTAGTT
TTGTTTAAACTAAGACTATACCTTTCTGTCTGTAGTTGATTAATATTCGATTTAATCATT
TCATTTAATTTGGGTTCCACTATATGAATGACATCGCCAGCAGTAGTGGCCATTACCTTT
AGTTTGTGATGCACGTTCGTAAGAACGTCCTCCCAAATGTTAGTAACTACGTCCATCTAT
TAAAACCTCGACAAATTAATTTGATTTTGTAATACACACATGTAATATATATCTCAAATA
AAATTTAGTGCAATAGCCCTCCCCGAAGGGAGGGCCATATGCTCTTATTATCGATTAACT
ACACGATAATTATCAATGATAAACTCGATGTACTGCTCGACATCATGTAAGAAGCAGTTG
TAACTATACCGATCAAGTTTATTTAAAGCTTCAGTTAGAACTGGTTCGATATAGATATTT
ATTTCAGCTATTGATGGAATCTCAACATACTTGTTTTCATATTCGGTAAGCTGCTCCTTT
ACCTTAGCCATTTGCTTTTTGTAAATGTATTCCATCGACCATTCTTTATAAATGGAGCGT
GCCCCATCAAAGAGATGGTAACCAAATGCGATGCCACCTAGTACCAGAACACCAGTGGAA
ATAGTTGACACATTCATGATATATCTCCTTTTTTCAAGATTGAATTCATATTAGTAATAT
ACTGTTTAAATATGTTTGAATACAAAAAAAACACAATATAAAGGCCTCCCCGAAGGGAGG
CCTTTATGGTAACTTTGTAAAAGACTTATTATAGTTGAAAGTCTTTATCTTCTTCAATGA
TTTCATAACCTGGTAAATTAACTATATTTTTATTTAAACGGACTCGTTCATCTACTAGGT
ATTTAACTACTTTAAGTAATTGACTTATCGACAGGTCATTATTTTCATCAGTATCTGATT
TAAAAATACGACCACTGGTAGGTGAAGCATTCGTAAAACAAATACCTTCATTATCTTTAT
AAATATCATAATACCATACGACATATGGATTATTGGTATTACTTATGACAAATACTAAAA
ACAGATCTTCATCATCACCTTGACCACTCAGGATCCCGATATTTTTTCGATGCGTTATAC
CACCTGTAATAACTAGGCCGTTGACATCATCACTGAATTTAAATATATCATACCCACCTT
CAAGTTTACCATTTTCAAAATACTGTAATAGCGCACCACCTACTACAGCACCCCATGAAA
AGAAGTTATCTTTATTTAAATGCTCAACCATCTTAAAAATCCTCTTTAGGTTCAGTGATC
CGAGAATAAAGAACAAACATATCAGTTAGGATATTTCTATCTAGAATGGTACGTGTAAGT
AAGATTCCATATTGAAATATACCTGTAGTATACAAATTATAAAACAACATAAAAAGAGAG
AGGATAAATTCCTCTCTCTTTATGCAACTAAATAGTTTTATTCTTTTTGCTTTTCTTTAG
CTTCACGTTTAGCTTTGAGTTCTGCATTATTAAGCCGCCAAGCAGCTTGCATAGTAGCTA
GATTGACAAACTTAGGCTGTTGCTTATTTTTAACTTTCATTTGCTAATTCCAAATTTATT
TCAAATGGTTAATCTAAACTATTAAATTGCTTCATATTCTTCATGCATAATTTAATCCTC
AGTCAGCGTAGTATAATCTACATAAAGATCATTGAGTTGCTCTTTTAATGAAATATTTAT
TTTTAATTCATTAAGAGTAATCCTATTCCAATGTTTACGCGGATTACCACATAGCATACA
ACGAGACCTACCGCAGTTAAACGGATTATGTTTACGCCATTTACCTTTAGCCTTTGCTGA
AGGATTAGTGTCATTATAAAGACTCGCGGCTAGTGAAACATAATAATTAACTATACGAGT
ATTGTGATGACGATTAAGTTGTACAGTTAGTTTGTTGCTTGACATATTTATACTCCACCT
TAATACCCCTACCCCAATATGGAGTAGGGTAAAGTATGTAGTAGAGTAAATTGAATACAT
TAACTTTTAACATTCTAAACTTCATTAATTTTCCACAATATCCTGGATGGTAAGTAGACG
TTCACGCATCGTCAGTAAGAAATTACGATAAGGTTCACCGGCGTCCAAAAAAGGTGGCAT
GTTTAACCTATTAGTAACCTCTTCTAGAAATTCTTTATACTTATAAAGATTAATAGTGAA
TTTATTACAATCTGGATCAGTAGTAGTTGGGATAATATAATTACGAATAAGGTAGTAAAT
ATGAAAACTGCTACCATATTCAATTAAAGTATAAGGATTATTATCAATGAATTTCTGTGC
TTCTTTTTCTCTTAAGGCGAAATCTTCTCGTTCAACGATTTCATGAAGTAGTGGTCCTAA
GAACTGCATTGGAATCACACCATACGTTTTAGTGAACTTCTTAATATCCTTATATATTAC
TTCTGGATTAGGATATACCTCACGAGTCCGACCAATATTTTTAACTAGATATAGTTTTTC
TTTTACTAAGATATTTTCGATGCATAGTTCATCTTTAAATAGCATAAAATATTTTCCTTT
TAATAAGATGGTATTAAACTTTTTTCTTTATTGACTAATATTTTCTTCGTTCGGTACCCT
GTAGTTTCGCGAATGTGTCGATCTTGTACTGGTCAACAAAATGCCAGGCTAGATCGGTAA
GTGCATAGATGTCCGGACGTTCGTATTTACCGAAGTCTGGTTTCTCACGACTCAATTGAA
AATCGATGAACTGATCTAATATAGGGCAACGATCTATCAGATCCTGTTCAGTAGCCCCAA
CCTTATCATTAACCAGTTCTTGGATACGTTTATAAAGGATAGGATCTGTTAGAAGAGGCA
GTAGATCATGGAATACCATCGGTGGATGTTCCAGACTTTCACGGACCCACATTGCAGCCA
ACAGAGGACGAAGAGAATAGAAGTATTTCTTCATCCGGACCTTCTCATCTGACAACCAAC
GCATTTTGGTTTGTTTGGCCATCGAGATATAATGCATGTAACTCGATTCAGGGATGAAGT
GCTTTAGCGCAGCATCACGGATTAACTTGTAGTCCCGATCAACCTTACGGTAAACGATTG
GGGAGTTTAGCCATTCCAAGAGAGTGGGGTTAGACTTGTAAGCAAGTTGAAGAGTCTTAG
TGATACTCCAACCAGCCAGGTCTAGTTCTTTAGACACCATCTCTTCAATAGTATCACGTG
CTTCATTATCCACACGAACATAATGACTCGGATGGTTAATGTAAATGAAACGAACGTCAT
AGTCACTATTGGTTGAAGCAAAGCCCCATCCACGACTACCGGACTCACAGGCATAGATAA
CCTTTACACCATATTTCTTTTCGATGCGATTAAGTTCAGCATCGATGATTGCGCGCATCT
CATCGCTAACTGGATGTCGTGGATCAATCATGTGGTCACCTTAATCATTCGAAATTTCTC
TTCTTTACCGATATCCAGATACATGAACATCGAATTTCTCCAAGGGAAATAAAGCCTCCC
CGAAGGGAGGCATTTATGTTAGACAGCAACTTTGCCAGCAATGTGTGGATGATATTCGTA
GTTCTCGATAACAAAATCATCTACACCAATTTCCCGAATATCTTTACCAATAGCTTCTTC
TTTAAAACGAATGGTAGGTAGAACCTTCGGTTCACGAGTTAATTGAAGATCGGTTTGTTC
TTGGTGGTTAGAGTAAATATGTACATCACCACCAGTCCAGATAAACTCTTCAGCTACCAT
ATTCATTTGGTTAGCTAACATATGGGTCAGCAGACTGTAGAACGTGATATTAAAGGGTTT
TCCTAAGAAAGTATCGACGCTCTCTTATGTTCACCCAGGGTCGTTAATCCTGAGCCGTTT
ACTTAAATGACTTTATAACCAGAATACTTTGGATTCTTGCTGTTAATTCGGTATGTTATT
GTACCCTGTGATACTCCGAGAGCCTTAGCTGCATCGGCCTGGGATAAATAAACAACACCG
TCCACTTCAAGTTTGTTTGAGTTAGTTGGCTTTATACCGCGTTTGTTATCCGTCAACTTT
TTTCTGGTTTCTTCGGAATGTGTTTTACCGTAGAATGGATTACGTTCGCCTGTTCTCTGA
GAAGCTATCTCCGACATCCTGGCTCTTGTCTCATCACTAGCTTTAATGCCAAGACGGTAA
GTGTTACCCGTGTGTAACTCGGATAGAGCTACCTTAACTTCTTCTGTGTGAGTACGACCA
TACATCCCATTCTGCTCACCTAGTCGACCATACTTTTCCTGACGAGCCTCGGCTCCAATC
TCATCAAGTCTGATACGTATTGTATCACTCCGCTTCTCGATGATTTGTTCTTTGAGGTTA
TGCCTGGTTAAGTTATCACCACCCTTGGAGTGTCTACCAATATTAAGAAGGTTACCTTCT
TCTTCATGCTTGGTTATCCACATCTGTTCGAGTGTAAATGCTGATTCCCGACTTTCACAA
CGATAGTACTTTAACGAGAATCTTTCACCGTTATCGTATTCTTGTTGTAATGGGTAGTTA
TGGTGCTTGGAATTACTAAGGTCTCTGAAATGTCGTTGAACGCGCTTAACTAAATCTCGC
GCACTTCCGACATATACCTTTCCTTCTTTGGTGAATAGGCAATACACATAAGCTACGTCA
TTAATAACACCCTCCTTATCTGGCGGAGTCATTAAGTAAACCGCTGCATGTCTCCATGCA
GGACAGACTATATCACGATCTCTTAAAAAGAGACCCCTACCTTTTCGGACCACTTGGTCC
TACTCTACTCCCTTCCTCTTCTCAGAGTGGTTTCGATAGTCGTTAGGCATTTATGACCGT
AGTCAATTTAGCACGGGATTGTCTCATAGAGAGTTTCCCCGTTTAGATAGGTTATTCGAC
TAGGATTGCTCCTAGAAGGCGCTAGTATTTAACGCTGGTAGAGTTGGCAACTCAATGCAC
GTTTAGGAACACCAAGTTTATCCCACAGTGCGTGATCAGTTTCCGGTTGCGCACCTTCAA
GGAAAATACGTTGAGCTTCGGTGTCATTAAGTTGGGCCCAGTCATACCGTTCTTCTAACG
TCAGTTCACGAGTCCAGAACTGGAAAAAGCTATGGCACGGTGGGAGTGACTGTTCATCGA
CAAGTTGTGGTGCCCATGCACAAACGATGATCCGACGAGAATCAGGGTTAGTCTTAAGTT
GATTAATCACGTCTTGAATCTGATCGATACGTCTAGTAACAACACACCGATCATTCTCCA
TGCAAGTACCTGGAATATCAACTACGAAGTCAAAGCCACGTGCCTCGTAATCAACCCACT
CGTGTTTTGGGATCACACGAGTATCTTCAAGATCACGCCAAGTCTTACCGTAAACAGCAC
CTAGATCGCCGCCGACCAGGCGTGCTGGTTCTTTATGCGTCAAAGCACGATACAGCCGTT
TGTTATATTCAGTATGAATACCTTCAGGATTTTTGGCTTCATCGAATCCTTCAGTAGCGA
GCCTATTCCACGACTTTTCACTAACTCCAATGACGTATTGGATTTCCTCACGGTTAACTG
GATGTTCATCCCAGAAAGCCTGAATAGTCTCTGGGGCCATACCCGACTCGAACTCCACTA
CAGTCTCGATGATTGGAGTAGAGTCATCGTTCATGCAATGGACGTACATTACGAATGGGG
CACCAATGAGGGTAGTTACTGCTTTGAGAACGGCCTCATCAGTCATCGGCGCATATAACG
CGGTATTTGGTTTAACCCATTCATTCCAAATGCGTACACCATTTTCAATTAAATAATCTA
CACGAGTATCACCAGAAAGCATCCAGAATAATTCATGGATAACGGATGGAAGATGAATTT
TCTTAGTGGTTACTACGGGTAGAACGTTGTTACGTAAACTATAACGTTCTTGTTGACCAA
ACATGCTTTGGGTGCCAGTACCAGTACGATCACCTTTATGTACACCGAGTTCTTTAACTA
AACGAATTGAATTAAGATATTGTTCCATTACTTGATCCTTGGATTGTCCACTAGTCCACA
GTTGAACCAGAGATGCTTATCATGGCGGCGATCATCACTAATAGACATGATCGTACCCTT
ACCATCTTTAAAGTTAATAATCAATGCTCGAACTCGGCCATTGGCAAGAGCATTGAAAAT
TTTGTACGACCTTACACTGAATGCGATTCTTTCATCAAGACGATCCATACTTTATTCCTC
TAGTCTTACTGATAATCACAGATCAACCTCTATTCGTTTGTATTTGATTTCAACCGGAAT
ACCTAATTCATTTTTTACAAGATTAAATGAGTTTTTTGATGGAGTGTATGTAGCAACTAT
AACGGCTTGACTATCAGCTACAGCTACGTCTCGCCATTCCATGTGCATGTTATCGAAATC
ATCTTTACCCATGATCAAGTAATAAGACGTATCATTATGCGTAATGGTATTTTCAAACGG
TCTAATTACAAAAGCACTTAAACTAGACGGTATACGGTGTCCTACTTGTTCAAGTGTAAC
TGTATACATAAAACCTCCTAGACGGTATACGGGAGTCCCGAAGGACTCCCTAAGCGCAAC
TAACCAATCGGCTAGTATCTTACCTATATTATTAGTCTATCAGAAGTATTTTTATTATAG
ACCAATACGAGTCAAAATTTCAGCCCAACCTTTCTCTTTGGCATATCGCAATAGGCTATT
TTCAATATGGGTTTCACTATAGACAATAGGTTCATGGCCACCTTGCACATGATACATTGT
TAATGGTTCCACCACATGTTGACTAGACCAAGCATCGTATAAACGATCATTGATTTTAAT
CACCACATATACCATCAATTTATCGTCGATGTCAAGATCAATACCTGCTACAAATGCATT
AACATTGGAACGCACTTGGTCAGCAGTTAGTTGCACTTGCAACCCGCCATGTACTTCAAA
TTCGATATTACAGATATTCTTATTTAACCAAGTATACACTGCCTGATTAGCATATGTCTC
AATTGGATCACTACTACGTAAATCTAATAATGGTCCATATGTTTCTAATGATGAGACACT
GGTATCAACTAGGTCATTCAATGAGTATTCGGTACTAGAACAACTATCTAAAACACAGAT
AGTGTTATCCTCGATATGGATAGGTAATACCTTGAATACCACATTAGCAACATTAGGTAA
TGACGTATCTGGTTCAGAACTATTGAATGGAGTATAAAATAATCGATCAGCCACTTCAGT
ATCAATAGTTGATACATCCATTGGAATGAATCCATTAAGATCTTGTTTGATTACGTCATT
GTGTCCAATTCCAGAAAAAGATAAATACATTTTCTCAGACATATCCGTACCTATTTATTT
ACACAGATTAGAGTATGTTCTACTTTACTGGTACTAGAAGAAAAATAACGTAATAGAACA
CCATCGCTGATTTTTACAGTCTCTACATTATTACGAATAGCACTTGCTTTAGCTAGTTTT
TGAATACGCTCAATCGCGCTAAAACAAGTAGTCATGTCAGAGAACTCAGTGGTTGACTGC
GATGCACCATTGGCTTGATAACCAGCGATACTGATACTACTAAAAAGAATTGCTAAAGTT
GCATTCATTTCTTACCACCTTTAATAACAGTGAGTTTTGGTGGACCAGTAGGACGAGAAG
GTTTATTAATAATAATCTGCGAATTGGAAGAATTATTGAAATCCTCAACCATCTTATTTA
CATCATTGTCAAATGAATTAGATGTATTATCACCCGAGTCCACAGTTGATTCACTAACCA
CATTTGAAATAGGGTAACTGAGGAGTTCCATCATAAGTACATTGATGTTATCCTGATAGT
TTTCATTACGTGTACAATTAATAACCCGCTCACACCAATCCATAATTAATTCTGGTGCAT
TCTGTTTAATTGCAGTTACCGCTACTCTAATATGTGCATCTAATTCTGGATTACTAAATT
CAGTTACTCTGTTAATGGCGCTTTCAATCATCTGATCATGCTCATTCTCAGATGTATTCC
TTACAACTAACTTACCATTAGTGGTATCGGCTTTATTTTTAAAATTAACCTCAGTGACAT
TACCTACGGTAGTTAATACTGGGTTTTTGTCAACTACATTATTAACAGTTCCTTCTAATG
TTGCACGAATAGTATCTGCATTGAGATATCTATCTACTAGCGGCATTTCAAAGAATGCGT
TAGAATTATCATCAATAGGTGTAACTACACCCAATACTGCATAAAGAGGAAGTTTAATAC
TGACCCCAACTCCATTTACTGTTACATTGAAAGAGATATATTCGTCACTGATATAAAAAT
TCTTTGCACAGCGTGGATGGATATTGAAAGTGATAGTGTGATCTTCTTTCGCATGCTTTA
GAGCTACCTCAGATAAATCAGGTACTAATTGAGTATGTGCTACAATTAGAATATCTCTGA
AGTTATTCTCAAGAAACCAATCATAGGTAGCTCGAAGAATATGGCCAGTAAGTAATGATT
TTACCTTTAGCATATTTGTATTACCTTTAAATAAATTATAGGAAGATATCTTCTACTGAG
GGCACTATATTCTTTTCTTTTACAAGTTCAGTAGATTTGGTATAATGTGCATTAAATAGT
TTCTTTAAATCTGCTTTTTCATATTTTACAGTATCTGCTAATTTCCAAATAGTAAGACCT
AGATGAATACTAACTGAATTAACATGTTTTACGCATAGATCAATTAATTTGTTAAACTCT
TCTAATTCATTTTTAATCAACGATGCATCTAAAGCCTGGTGCAAACGATTAGATAATTTA
ACTTTATCTTTGTCAATTGTAAAGTTATTTTCAATATCCTTAGCACTCATCGGTGGTTCA
ACTTTATACCACACATTGGTTAGTTTACCGTCCACACGCGTCGTGATGGGATGTTTATAA
TCAGTCCAACTTCTATGTAGATTATGTTTATCATCAAGAACATCTACAGTAACTTTCTCA
TTACCTGTAAAGAATGGATACATTAGGTTGCCTTTATTTTATCAATGAGTAATTTTAATA
GTTCATCATTAGTTAAATTGTCTTTATTTTTAATAATGTCTTTTAGTTTAAACATATTGA
AAGTATACCACAACCACTTATGAATTATATAACAAATCACTATAACTAATAATAGTAGTG
AATGTAATGCTAGCATTATAACTACCTATTTTGATATGACTTGACCAAATATGTAATATA
GATTTCAAAAAAAATTTAATGCAATAAAGGAGAGCTAAATAGCTCTCCTATTTATGCTTA
TTTATTTTTCTTTCTTGACTTATCTTTAATAACTAAGTCTAATGCTTCAAACATTTTAAC
AGCAGTTAAATGGTCGATAGAGTATGCCTTTTTACGTAAACGTCCAATAGCTACTGTATA
CTGGTATTCAGATAAACCATCACTCATATTGTCAATAATTGATAGTGCTTCTTTATAAAG
CCTGTTACACTTTATCGTGTGTTTGATGGCATAATAACCCCAGTTAATTAAAGCCATCAT
TATAATCAGAAACAAAATACCTATAATTACTGTCATGATATCATTCATTTGCTGTTTCCC
TATTATTACATTTTAATATAATCAATGAGTTGATATCGTAAATAAAAGATGTAAAAATAT
GTTGATTTGTTATTTTAGATGATTCTTTATCTATAATGTCTTGGACAGCATTTTGATAGA
AAAAATAACTTATGCCATTGGGCATTTTATCTAATACATATCTAACCTTATAAACACCCA
TCCAGTGAATATAATGATTTTTTATATTAATAATGAAAGTTACACATGTTGATATAGCCG
TTAGTGTTATCATTGATATAACAAATACTTTATACATTTGCAACCCACAGATTCTCATCT
AGATGAAATAAACCAGCCCCGAAGGGCTGGTATTTATTTTACAACCACAGCTTTACCGTA
GTTGTTATCCATACAGGTTTTAAATGATGTAACGTATGCATCACGTGGGCTTTGTCTACG
TGAATACACAACGTGCACCTCTTTAATTGCTGCTGGGAGAACATTGTAAAGAACGTTATC
TTTGTTCTTCACGCCGTACCCTTTTAGGGATCGTAGGTTTTCTTCCCATTGTAAAATGGG
TGTTTTACGCTGACGTGCTTCTGCTGTGGCATGTGCTATAAGTGCAACACCATTACAGTA
ATTCTCGTCAACTACCAACGATGCAGCTAGTGGGTTTGATACGTATAACAACGTACTTAA
TACGGCCACTAACTTCATTACGTGTGTTAAACATTGCATCACGTTGTACCTAGGTTGCTA
AGATCAGAATATAAAGCTACTAAAATAGCCTTAAATCATTCTACAACCTTAACGTTACCA
TCTTCAATAACTAGATCTTTACCTACAACATCGGCAAGATCTAACTTATCCTTTACTTCC
AGTTTCTTGATACTGTGTACAAAACGGAATACAGATAGACGAAGAATTAGTTCTTCTGGT
ACATCAACAACATCAATAAAGTATTCTGATGGGTTGATATCACAATTGACATATTCCCAA
CTTTGTCTTAGTTCAGTTTCATTCTCAGTTACTTGTGCATCAAGAACTTCTTCAAAGTTA
ATATTGGCAATATTAGTATCATTATCATTCAGATAAGTAAATTGAAGACTAGCATTATTA
ATGAATTCTTCTTCAACTACAGTTAGTGCACGTTGAGCGTGAATGGGTAGATGATATCGA
ATACACCCATTAATAATAACCTCATTTGCCTTTTCAATATAACGTTCTTCTTTGAATTTT
ACATTAGATACGACGTAATCTAGGATTGCCTTGTAATATTCTTTATGATCTGGGTGGCTC
TTTTCAATCCACTGTCGGCTACTTACGCTAATTTTTTTCTCGGCTAGAAAATCTCGGAAT
TTAGCACGATCGATATAAGCGGTACGTTTTTTCATCAGTAGTTCCTATTATTTAACATTT
CTAGGTGTTAGTTCGATATGTGAACAGATCACATAAGATGCACATGCCAGTACATCTAAA
CCATAATACTCATCATAATGGAAATATCCTCTGAGATTACCTTGTTTAGTAAAGGTGTAT
TCATCAGCTAAATCTAACTCGTTACTTGTCTCTAGCACATTGATTAATTTATCATATTCA
ACTTTTAGTGCTATATCAGTACAGATTATTTGCTTGATATATTTATCAGAGTTTGGATCA
TTTTTATAATCTAAAGTATCAGTTAATACAGTGGTTAATGCTAGAATAACATAGTCATGG
TAACTTTCCGGTTGTTTAATCTCACCTTCAGGTATTTCAATCCATTCCCAACCATGTCGT
AATATATTTAGCATTACATGCCACTTACTGGCTTCATTACTAACCACAGCTATTGGTGAA
TCACTATAAGGTTTATACAAACCTGTTGTTTTTAAAAGATCTTTTTTAATAGCAGCTATA
TATAACGGGCGGTTTTCTGACATTTTTATAACTCACATATATTTCAAATGTTTGCCAATT
GGTTTATTAGGGTTATTCATATAACCACGACCTGCTGCTGCCCTAATTACTACTTTCTGT
ATATCTGTTAATTTTCTATAATTATCACCAATTAATTTAAGTATGATACTTTCTATAGAA
TCATTAGTTAATTCAGAAGGATCTGTTACTTTGAAATAATTATATACTAATTCTCTTTTA
ACTAGAAAATCAGCTCTTTGTTTTTCTTTAATTCGTTTATAACGTTGAGATACATGGATA
AATGAAAATAAAACAACTATCCAGAATAAGGTCAAAAGTAGGTACTCATAATCTACATTC
ATTTTTAATCCCTATCTTTTTTTCAAATTATCTTGTAACTCAACTAGTGTTCTGTTTAAT
AAACTAATTTGTTTCTGTTGTTCACATACAAGTTCACTTAACGTATTAATCTGCGCCTGT
AGTTCTTTAGTATTAGTATCAATAACATCAAGAATATATTCTTTATTTTTCTTAGGCATT
CTTTGCTTCCTCAACGTTAATTCACCGAACGGCAGAAAAGATGACTGGGCAGCCAACACT
CTTTTAGAATGTCTTACGAAGTTTAATTGGTATAAGCATTAAATAATATCTCTTGATGCC
CATGTACCATCTACAGAGTCATTTACAAATGGTGCACGATCACCAGGTATTACTTGAGCC
CATAAATCCACCTCAAGTAATATCCGACGACATGTATAAAAAGGCTCTACTTCTACCCTG
TATGAATTATCTTTAGGATCAATATACACAGTAGCAACTAGCTTAGGCTGGGCTCTTCGC
CATTTATCCCATACTTTAAGAATTACTTCGCCTTCCATTCTGGCTATACGTGGAGTTATC
TTTACCTCACTAATGATTAGCCCATCTGGACCTACCACTCGTTTAGGGGATAGCCATTTA
TGATAACCAATATCATTAATCCCATTCTTTGAAAATACCGAGTCTTCAAGATATCCTCTC
ACACCAGGGTATAACGGAACCATTGTAAGCATATGACACCTATAGTGATCCATTGGATGC
CCATAAAACTGAATAAGGATTTTGAGTTAATTGGAATCTAATTGCAAAGTTTTCGATTTC
CCAAAAACGATCAGTCTTATTCCACTCTAATTCCTTTTTCGCCAGATCAACATTAATTGT
TGATATAATACGATTACGCTCTTCTTCAAATACAGTAAACCTGACGATTGCTGGAAATAA
GCAATCTCTTTCTTTATAAAAGTTTAAAGTATTCTTATCGATATAACCTGGTCCTGGAAT
AATTTCAAGGATATTAAAATCAGTCCATCCTAAGTCTGGTGTACCTAGATCAGGTAAATG
TTTTAAAACCCCATTGCGTATAAAGGGTTTTCCATATTCTAGTGTGACTTCATGAAAAGA
AGAACCATAGATTTTCATACAACCACCATGCGTTTTAATTCATAGGGTTAGTATGTTTAG
TACTGTTTGAATTTTATTTCATTACAGACGATCGCTTTGTTATGCCTGGTGTTTGAATAT
TTTAGACTAGTTTCAATAAATGCTTTCTTTTTCATATATACACTTACGCAAAATAAATAG
GGACTCTCATTAAGAGTCCCTTAATATTAAACGAATAATAACTCCAGTACTAATACTGGA
AATTTATGCCGATATTGTGGATGTACATACGTATGAAATTTATGTGTGCCTAAATGGAAT
TCAACGTACAGTACAATAAAATCTTTAGCTGCATTTCGATCATGTACGTAAACAGTGAGT
ATTTCACCATCATCATCTTTCTTACGTCCATAATCAAAATAGTAATTTTTGTTATCAGCT
ATCCCTTCAGCATTTAATTGAATGCCTCTTACATCGACAACATTTAAACTAGAATCTTCA
TCAAATAAAGATCTAGTACTACCATGGTAAACCATTAATGGATCTAATTGAATCTGCATG
TGTTAGTCCTCTACTATGTAGTGAGGCACATGCCTCACTTATATTGTTTTAATTTTCATA
TTAATGATCAATTAAAGTTAACAGTGCTATAAGCTTTGCCTAAAGCTGTAAATAGTTCTT
CATTAAGTTCATCAAATTCTAACCAACCATTCATTTCACCTAAGATAGTTTTAACAATCG
TAAGATTAACAATCTCAGAGCTAGGAATGATATCTCGAATTCGCTCATGGAAATTAACTA
CTTGTTGAATACTTACTTTAGTATCATTGACTGAGATATCATCATCAGTTAACTTATTAA
TAGCCATCATCGCTATACGTGAGAATAGATCAGTATGTTCTTCCTTAACAATTTCCCATG
CTGCATTAAAGGAGTCAAGAACATCTAGATCATCAGAATTTAGTTGCTTTTGCATTTTAA
CCTCTTAAACATTATAACACATATACCCCACAACATAAGTCATGGAGTATATGCCTCGAA
GGGTACGTAATAGAAATACTACTACATAGTATTGTATTTACTCATTAATAACAGATACTT
TGTTATTCTCAGCTTTACTTTTAGCCGATAGCGCTAAATAGAGACAACTATACCAAGTGA
AGTAGTCTCTTGGGAAATAGCCCTTTAAAAAAGTAGCCATATTTTTAGCACACGCTTTAT
TAATATTCTTATTAATGATTTTCCTCTCATTCATTATTTTACAAGCTAACAATGAATAAT
GTAGTTGATTATTAGTACTTAGGTCTTCAAGGAATAGTTCTTTCACTGCTTGTGGCTTAG
AACTTAGACACATTATAATTTCTTGAACAATATAGTCATTCGTCACTGAATGAATAAGTT
CAAGATAGAGTGAAAGATTAATCGAATACAGATCTTGTTTATTATTGAAAATGTATCCAT
TTCTATCTAATGTAACTAATTTCTTATATGTGGGTGACATCTTTTCATTAACTATTTTAT
AAAAAAGACTAATCACTAATTTAATCATTTTAATAAACTTCCATCTACTCTATAAAAACC
ATGGACATCACGTTTACCATTTAGACTAGGTATCTCATATGGATCAGTTGATGGATTTAT
CAGTTTGATTTTGCATTTATCTAAACGATCACGCAAGCTGATATTACCAGATAATGTTAA
ACCACGATACTGAATATCGACATTATCTAAATCAACAATGAGCGTACTAAGCTCCCCATT
TGTTAAGTATGGTACCACAACCGAGTCAATTACTTTTGCAATTGTTTTAGCTTCAATATG
TATCCAGTTTCCAATATGATTAACAATCTCTGGAAACTTATCTAGATTTAATGTGATGCT
ATAGTCACACCTAATTTTGATAATTCCATTACTTGACCAATAATGCATTGCACTATTGAT
GCCACCTAGATGATTCTCCATAGGTGTGAACTTTAGCCGTCCATTGAAATCTATAAGCAC
TTTTGAGTTATCACACGCATCAACTATTCTTGACCGATACATAAACATATTAAGTAGTTT
TCTATTGATGGCCACAGTTGTACCATTAGTTAGTTCAACTACGACGATATCACCATCTGC
TTCATAAGACTCTACAATTAGAGTTATTGGGTTACTATCAAATTTGGAATTATCAGATAA
CCCATCCATCACAATCTTGTAATTACTATTAACTGATTTAACTTGATCTATTAAAGATGG
ATCAATGAAATCCTTAGTGAGTGATTTTGATGTGAAAATTACAGCCACATTTAGATCACT
ATTGATTGGTGTATTTAAAGGTAAGTATTGTTCATTAGAAAAATAGAATAATTTAAGATC
TTCTATATCAGTTAGATTAATAATATCTAATGAATTATTAGTTACTTTATACATGAATGA
ATCAAAATTTTGTTCAACAAAATAAATCATCCATGTCTTAGTTTCTTTGGTATAATAACC
CCATGCTAAACTTCGATCATGTAATTTACCAGATAGCATTCCTGTGAAGTAACCTGACAA
AATATCAATTTTATCAACATCTACATTTTCTCTGATGTATTTATCATTTAGATCAGCTAT
CTTTTGTTCTAATGTATCTACCTTTTTTGGTTTGTTAAAACTATCAATAACATTTTTTAA
GTACTTAAACATAATCATAAATTACCTTTTGAGTATAAACACGGAATAAAAGCGGTGTAC
ACACCGCTTATTGTTCAAATTTGATTTTGATATATCCACCACCAACATTAGGCTGCTTAG
GTCTTAGCCATCCTAGATTAAGATCCATATTCAAAACAAACGTATACGCTGTTACTCCAT
TTTCATCATCAACACTAATGGAGTGCCATCCATCATGATTTTGATCTTTAAGAGAATGGA
TTTTGGGATATAGTTGATTATGATTCAAATCAAATACTGTAACATCTCCAATTGATCTAA
TTGGATAACCGCGAGATCCTAAATGATGGGCTCCTCTGCGGTCCAGTGAAATAATAGCTT
CGTTCACGATAATTACCTCATGTAGATGTTTCCATTAGTATATGCTAATCTATAATGTAG
ATCTCAAATAAAATTTAGTGCAATAGCCCTCCCCGAAGGGAGGGCTTTATGACAACATTA
CCGCTTACTCATTATCGCAACTAGTTCAACTTTAAATAATGCTTCTTCGACTTTACGAGA
AATAGCATTTATTTGAACACGTTTATTATTTTCACGATCTAATGCAATTGTATTTTTTCG
TGTACAATAATCGTGTAAACCCAGTTGTCGCCTAACATAGAAAATAACAATTAATTTCAA
AACACGAATATATCTCATAACGCGCTTGAACATTTTATAAATCCTCAATAATTTAAGGAA
TTGCTTTTAAAGCTTCTTCGCGTTCAGCTAAAAGCTTATCATATTGTATTTGTAATCGGT
TAATTTCCGCACGAGTTAATTTTGTCTTAGCATTAGGTTTCAACTTATGGTTAATATCAA
CGGTAATACCATGGTACCTAAATAGAGCTTCCATTTTGGCTATTTTCTTTTCAATATCAT
CCATAGCTAAAATCCAAAAAAATAATAATGACTAAGGGGACCAGAAGGTCCCCTATATCG
TTAGGCAGTAATAGCGCGGCGGAAGACTGAATCAAACTCTTCCTGGTATGCTTTAATCAG
CTTTGCAGTTTCTTCACAAATGGTATTATTACCATAAAATGCATCTGCAAAAGATTCTTG
CTTACGTACTTCACAACCGAATAGCTCAAGACCATTTTTTGCTAGCTCAACTTTGAACTT
AGCTAATTCGGTCATACCAATTGGTTTGGCCCGCCCTTCTTCGATACCATTTTCTTTTAG
TTTAATATTATCACATACACTGCAATGACGAACATCTTTTTGTTTGTTCTTACGGAAGTT
AGGACTTGATTTAGTTTGTTTTGCAGCAATGTCCATAAAGGTTTCCCTAGAATTTTTACG
AACCGCAGCTTCATGATTCTTTTTAGCTCGATTTTTCTGTTTCTTCTGAACCGGCTTCGG
CTGTACACCCAGCATTGAACCAAGTTCACTCCAGGAAGAAATAGTATTGCGTGACATGTA
TTAGAATCTCCTATATATGTTGATTATATCACATTTGTGATATACTGTTTAAAATGGTTC
GAATATATGAACCACTATGATGGTCAATCATAGATAGAATGGTTCATGAATAACAACCCG
ATAACGAGTGTTATATTGGTTAATTGGAAACATATGAACAATCTTAAAATGCAGTTCAGA
AACCAAATTAAAGAATTCAGCACGCATGTTATCAGTTGAAATATCAAATGAGATTGTATC
ATTAACTGCACAAAGTTCTAAATCAGATGCCTTTGTAATTTCAGTTAACCGACTGACAAA
CTTTTCTTGGTTAGGACTAAGCATTATTAAAGTCTCGCTGTTGTGATACCAATTTGATTT
TGATATTTACCATTACGTTTTGCATAACTAACTTCACATGGGGCATCACCTCTAAAGAAC
ATAAACTGGGCAATACCCATATCAGCATAAATTTTTAGAGGTAGTGTAGTGCAGTTTGCA
ATCTCAATAACGACTTGTCCTTTGAAACCTGGTTCAATAGGAGTGACATTAACAATTGCA
CCTAGTCGTGCATAGGTTGACTTACCTGTGCAAATAGCTAGGACATCATCTGGAATATTA
AATGATTCTACAGTATGACCAAGAATATAACTGTTAGGTGGAATAATACAATAATCACCA
GTCCAATCTACTAAGAATCCATCACTAGGATCTAATGGGTCAATAACTGCACTTGTTACA
TTAGAAAAGATCTTAAAATTACGGGCTAGCCTCACATCGTACCCATATGAACTAAGCCCA
TAACTAATAATCTTTTTACCATCTTTTTCACGGACCTGATGACCAATGAAATCCGTAATC
ATTGGTTCACGACCTTTAGATAAGTCAGTGATTGTTTTATCAGATAGAATAGCCATTTTT
AACTTTTCCTGTTAAGTAATCATCCCCAGAATCGGGCGATGAAGTGAACACACTCCATCT
TTTTAGTTTCTTCCAGGAACTTGAAATAACCATCATCCACACCATTGCTCAAAAATAGCT
GGCGATAGGTTTTTTCCGAAATGACTTCACCATTAACATCTTGATCGAACATCGTTGGCT
TCTGATCATAATCGAAGTTCCAGAGGTCTTCCATATTGAAGATCATTGTCAGTGGGTACC
AACCATCTTCACTATAATAGGCATATGGTGTTAGATTGTCCTGTTTGGAATTGACAATTT
GTGAAAGGCGTCCTTCAAAGTTGTTCCATACTGGATGCGAGTCATCACTGACGAGTGTAC
CAAATCCACAGCCTTTAAGGGTCGGTCGACAGAATCGGTTGGTATACTCGTTATTATCGG
TTAACCAATCCCAAAGATAATAGCAACGGCTAAAGTTAAGAGCCTCGCATGTATGCATGT
CAATGAATTTAAGCACACGTGCACCATCTGAGACACGTGTAGAACGTTTGGTAACTACAG
GGAAAACATCAATACCCATTTTAATCATCCAGTTAACAGAATTTTTCTTGACCAGGGATT
TTACCGTCCATAATCTCTAGTACCCAACGGCCAGTCATCAACTCACGTGCAACATCACAT
GCCACGCCGCCAATCTTCTCAGCTACTTTGTCTAACAGAATTGGATGATAGTTGTGATCA
CCCATTACTGCGAACTTGCTTTCCCGTTCTATTTCAGCAAGTAAATCAGTGTCAACCCAT
TCAACACGCCCATCTGGATAGACGATATTAAGACCATACCACTTACAATAGTTTTCGATG
GTTACAACTGGAATGTCATAAACACCTTTAAAACACAGCGGTAATTTACGATCTGAGTAA
TGAATACGTTCAGTCATTTGCCATTCACCAATTCAAAATAATTTTTCAGTAGATTTTCAG
TTATTGGACGAGTATGTTCTTTAATCGATAATTCCTTACTGTCCATTTCAATTTCATATT
CAGAAATTTGAAATTGCTTATGTGGCACTGAACCATCTAACATAATGACCACATCTGCTA
AGGCTTGATCCACTGCACATTGCAACAAATTATTAACTTGTTGATTATCAGTAAGTGAGT
AATCATTGATGGCTTTATTACGAATGTCGTATAGCCTTTTACTTAAAGGAGTGTTACGTT
CAATATGTTCTGTTCGCCACTCTTCTAAAATCTGATCTGTACTTAGTTTGGATAATGAAG
ATCCGTCAAGTTTCTTCCTAACTTCATTTTCTGAAAATGGTTTATCATGCCAGAATTGAT
GATCTTGTTTCATAGTAAATCCTTTTTTATAATTTAGAAATAGGTTTTAATACCGTATCT
TTTTCATTAAATGGATTATAAACTTTATGTGGATTAATCATCTTTAGTTGCTCATTCTTT
CTTTCTAAATAAGCATCCGATGGTTTAATTGTTTTAGTTTTCCAAAAGTCATCGATTGGT
TTTAACTTATCCATTTCTGACTGTTCGTTATCAAAAGGAGTTTCCCGATTTCTTAGTCGG
GTCGAATTGAATAGGAGTCGTAAACTCCCGTCGTAATTCCTCATCTAAATTCTTCTCTTG
AATTAATTTAGGAAAATGTTTTCTAACTGTTTTAATTGAAGCTATTAAATTGGAATAGGC
TTTTTCTATGGACATTTTTATAACTCAAAATACATTATAAAGACAGTGGCTAAAACCACT
GTCTAATTATTTCCCATAATGATCCACCTATAACTGCACTTAGAAAAAGCATTTTGAATA
TGGGTGGTTCTTCTTGTTCATGTTGCATATCTGATTTATTATTTTCGCTAGCCATTTTCT
TTTACTCTAATGCCCACCATATTCTCTAATTTTCTCACCATCCCAAAACTCATAACCAGT
CATATTGTGATTAAATGGCTTTATTTTAGTAAACCCACTACGATAAATCCAGAAATGACC
AGTGCCTCTTTTTAAGCGGCACTTATACATTTCTTTTTTCTCATATTCCGTGGGAGGGCT
AATTTCTAGAACTTCAAGTTCATCACCCCGTATACAGAATAGCGCTGGTGGATGCTCACA
GGATGCTTTCATACCGAGAACACCTGTTGACCAAACTGTATCACCTGGCTTAATAACAGT
GATGTCATTTGATATAGGTATTACAGGAATTGCACACATATTAGATGCTCCAGAGTAATT
ACCTCCAAAGAGCAAACACTGATATATTCATAGTCTGCTCTTTAGTAATATTTAAGAAGT
TGGAATACTCATACTGGAGATAAACTAATGTCCATTTATACTAATCTCATTGTGTATAAT
AATTCTTTTTGAGATTATCCATATCGTAAGAATCATGTTTGAACTCAAACTTCTTAGCTA
TCTCACTCGAAAAAGCTTGTTTACGTTTTTCTTCTTGTTCACGGATATCGTAAGGACCAT
TCTCTTTAACAAGATATTTTTTATACGGCCCAGTTCCTTGGATATATCCATCAGGAATAA
TGGTTGTGAGATATTTAGCCTCTACCAACCGGATGCACCGTCCATAACCAACTTCATTGG
CATGACGAACTTGGATTAGTTGTCTATTCTTGGCAATACGTTCAGATATGCATGGGAAGT
AAACCGGATCATCTCCTTTTTTACAGTTACCCATATCAACCTTTGCGTACAGAATTTTTC
TATGATTGGCTTCGAAGAAAGCCCGAGCTTCACGAATACGTTGGGACCGAATAGCTTCCT
TAGTTCTGACATTCTTTAGATGTGCAGGTTGTAAAAAAGGATTCATCTTTCGCACGTGAC
GTTGGATAAGTTTTGTCGTGTAATGAGGTGAATCATTAGCCATAGCCAGTAGGCTATTGT
ACATCTCAATCGACATGATTACTTGCTGCGGGTCATTATTGCGCTGCATGCTGATCTCCG
TATGTGAAATAATGCTTTACTTTAAGGTCATCAGGATTATAAATAACACGTTGGAATTCA
TGTTTCTTCGCTAGTTTAAAAGCAGCTTACTTTTCCTTAGAAACCTACACGACTTGCTTT
GCGTGGGCTACTGCAAGAACTAGTATTCAATGCTTCAGCTAAAGTAAGATTATTACCGAC
AAACTCAACTGGATCTAGACCATTAGCTAAACGAGCTGCAATCGCTTGTTCAGCAGTAAG
TGTTTTGAAGGTAAGAATATCGAAAGCACGGCCAGGTCGAACTAAAGCTTCATCAACATC
ACGAAGAGTCGGAAGATTAGTAGAAATGATAATCTTGGTCTTAGTAGATACTAGACCAGC
TGTAGCATTTAATAGCCCTGACATATTACTATTACCATCTACACGTTTAGCCACCATTTT
ATCAGAGTCTTCTGTAATAATGAGTGAATTATCCTCAACACCACGAATAAAATTAGTCAA
ACCGGCGTGGCCAAGTACATCATCTTGATCAGCTAGATACACATTACGATCATTATAACC
ACGAGCCCGAATAATCTCATTGATATAATTAGACTTACCAGTACCAGGAGGACCAATCAA
TAGTAGAACATTAGATTCAGATGCAGCGAATTCAGTCCAGATTTCAGCTGGAGATCGATC
TAGGAATGGATACATTTCCATCATATTTGGATTGTAGTTACGAACAGAAATAACTTCACT
AAAGCTATATAGTTCATTATTTTGATAGTTAACCCGAATTCTTTTAATACTAGGTGGACG
AGTCTGATCAAATACTTCTTCAAAACGTAATTTAAATACTTTGATCAGTTCTTCATTGGA
GTATAATCTAAAGTTCTGACTAGTGGTACCTTCCATTCCATTCATTAGGAAACCAATGAT
TCCTTCATCATCTAGTAATGCAAAACCACCACTAATTCTGAATGCTTTCAGATTTAAAGG
GCTTATAACTTCATCACATAGTTTGTAAATTTGTTCTTTGATGAATTCTTTTCTATCAAC
TCCAAAAGGATAGTGAATACGTAGATCTTCACTATCTGAACTAATTCCCTGTTCATTAAG
AGTATAGCGATATGCATTGGAAATTGCACGGCCATTATCAACCTCCATAGCAATGGAGAT
AAAACGATCAATCCACTTCTGCTGGACTAACGTAAGTTTGCTATTACGATAATTCATTTG
ATGTTCCTTTTCTAATTGATAATCTAATTTGAGACGATATAAACCCTCCTACTAAGAGGA
GGGTTTATTTTGGCTATTATAACTAATTCATTTGAGAAAATTAATTATTATATTCATCCA
TCAATTCAGGTAGACGGCTATCTCTAGGATCTAAGATTTCAGACCAATCAATTGGCATAT
ATGGCTCATCAGATTCATTTTTATTTAACCATTCTTCTAGTCTCTTTACATCATCGGACC
AGTATAGGTTATACATAGTTGGCACATTTGCATCTAATGCCAACTGCCATGCTGTATTGG
TGCCGCCACTAATTACAGAGGTTGGTGTTTTCTGTTCGCCATAAGCAATAATAGTTGAAA
CAGGTTCACGTAAAGTATGACCATGAATTTGAAACACATTACGAGTATGTAGTTCAATAC
CTGTTCTACCTAAACCATTGAAACTTCCCCGAGCTGCACAGGCCATTGCCATAGCTGTGG
GAATGGTATCAGTATATACAGAGGCATCATGGAACCAGGCTAGGTTTTCGATACGTCTAC
CTAAAAAACCATTTCTGATAATGTAAATACGACTTTCTACAGTGTCCCATTTATCAGACT
GAATTGCACCATACCAACCAGCTCTATCTGATCCATATGCATCACCTGATGAAATACCTA
TACCTAGATCAACAAATGTTCTTCCTGAGCGTATCATCAATTCTAGGGCCCATGTAGGGG
CATAGCGACTACCGACAATAGCTGTCATTGCCATTACCTAACCCTCATTAGAAGACGGAT
ATCACCGTACTTAAGAACGGCATTCCGGACTTGATCAATAAACGGTTTAAATTCTTTTCG
CTGTTGATCAGCCGTGAGTGTATAAGTTACTTTGATAGGTTCATTAGGAACATTGCAGAA
AGATAGATAACGCGATACTCGACGTAGATTACGTAGGTGATTCTTACCACATTTAGGCCC
GACTCGTTGTAACCGACCGTCGATGACCTTCCAATCATAATTAGCTTTGAGGTTTAGAGA
ATAAAAACTTTCAGTTAGATTCTTATAATACTGTAATTGTTTTTGAGAGATTTTATTAAT
TTTTGATTCATCTGTTACAAGACGTGGGCTATACCGTGAATAGCAATCTGGTTCTGACAT
ACGATCCCAGGACATAAAAGCATATAGACTCATATATACCGTTTTAGTAACTGGTTTAAG
CTCACTCATACCTTTTAGAATAGCTTCACCACTAATCCATGCATCACAGCGTGGATCATC
ATCATAACTACACCACCATTCTGGTTTCTTTTTCAGGTCATCAGGAACACCATTATGGTA
AGCAAATGGTTTTACATCCCAATAATTGCGAATATCAAGTAGATATGCATAAAGATCATA
ATTCTGTGTTAGTTCAATATCTTCGATACCGATCCATTCATCGTTTACTTTAGCTTGAGT
TTCTGAATATACATAATCACCCATTTTCTTTATTTCCTATATTAGATTAATATAAATGTT
TTATAACAACTATTTCAATATTAAATAACGTATTATTGATTGTTTTAATTTACTACTCAA
CCTGATAATGTGTAATACGTCTATCTATATATCTCTTTTCTCTGGTGAAGAGCATTAGCA
TTATTCCAAGGTAGAGTCCTTATTCTATTAAACATATAAGTAAATAATCCCTCCCCAATA
AAGGGGAGGGTATATGTTATCTTAATTCATGCTTTTGAAAGATCATATTTAACAGCTTCA
TCTTTCATAGGAGTCAATACAATAGTTACTTTCTCAAAACCATGTGTATCCCTACCATTA
TAAGAAATATCAATATTCTCCCAATCCTGATTAACTACTTCAGTTTGTAGAATATTTTTA
ATATTGATAGCATCTGTCTTATCAAACCATTTAAAAACCTTTGGGTTAAATACAAGTTCG
AAAGTAGTTTGATATGGATAGCTTTTCTGTTTAACAAGCCTACTAGATGCTAGTGATGTA
TTCTCAACGTTTCGAATAACATCAATCATTGCTTTACGGATAGTAAGTAATTTACTTTTC
AGAACCAAATCAATAAATGCTGGATCAGTTGTCATTACTTCTTTTACAGATGGAAATGTG
ATTTCCATAGCATCCTCTTATAGGTTAATTATTAAAAGATAAATCATAATCACTTTGTCT
GCGATTCGGATTAACAGGATCAGGATATTTTCCAGTTTTGATAACTTCTTTTATAGCTCT
AAAATCACGTTCTAGAAAATCTAGTTTTTTAACTAACTCTTTTTCTAATTCTTTATTGAG
TAGTCTTCTTAATTTGCTCATCTACGTATACTCTTACAATGAACCAATTGATTAGGCCAA
AACCTTTTGGTGTGATGGTAATGTCATCTTTATATCCATTTTTAGTGATTACATTACGTA
ATACTTTAATTGCTTCAGGTGGAATAAGATTAGCATTACCGGTAACACTATACTCGAAAT
AATAACCTACACCTGGTAAATAACCTGATGCAATCTGGTTAGTACCTGTGATATTACTAA
CGTGTTGGAAGTCACTAAAAGCATTCTTGTAGAATAACAAAATATTAGGAATTACTTCAT
CCCTAATAGTAGAACTAGTAATGGCATCCGCCGATGGAAAATAAGGTACTAATTCATCTG
TTACATTGGTGCTATTAATATCGGACATAATCTTTTCCTCATTATAAAAGGATAAATATC
TTTATGGTAATCCTGTAATATAGATTTAAATTAATTTATAATGAATGGCATAAAGGAGCC
CCTGGTGGGCTCCTTTAATGTAATAGGTCCGACCCCTATCTAGAGGTCGGTGAGTAGCAA
GGCACCGTAGTTGGGGTTCGTGTCGTTCCTGACGATACTTTAGTACCCCGGTTTATTTAC
GACTGGATCACCCCCTTAAAGTGCAGTGCGTTTTACGCAAACTGCCTTGCTCTATATCAT
TGCACTGTGCGTATTTTTATTCGTTAGCTAATCTTATGACTTAGAGTTATATTTACATAC
CCCTTAAGGAAAAATGCAATGATAGACTCATTTAGGAAGCTAATGGGCTCGCTATCAATT
ACTGAAACTGATCAAGAAATTATCATCAGTGGTTTTGATGGTGCTGCATTCATTAGAGAT
ATTAACAAGTATTGGCGAACCACTAAATTAGCTACGCAGTTATTTAACACGGTTAGTCGT
CGTAGTATTTCATTTTATAAATTCTTTGCTCCTGAAATATATTACATGCTAGAAGCAGTA
AAGAATTATAGAAGTAGATACATCTCTATTAAAACTGTTAATGCCATCCGTGAAGCAATG
CTTCAATATACCTGGTTAAAAAATACTCGTCCTGTAGATACCAACTCTGTACCAGGTAGA
CTCAATTTCAAGATGTTAGACAAATTGACATTTACACCAGATGAGTCTCAAAAAGCTTAT
TTTGAAAATTATAATTATCGAATTGACCAGTATGGTTTACGTGGTGATATTGTTGCAGGT
AAACCTGGTACTGGTAAGACATTCATGACGATGGCTATTGCTGAAATGGTTGAGTCGGAT
ATCATTATAGTTGTTTGTGAAAAGAAGTCTATTGATCTAATATGGAAGCCATCTATTATT
GAGATGTATAAGAAAAGACAAAAAGTATGGTCGACCATTGATGATAAAGCCTACAACGGT
CAAAGAATACTTATATCCCATTACCAGGCGCAAGATAAAATAATTGATCTATTAAGAAGC
GGTATATTCAAAGGTAAGAACATTACGGTGATTCTGGATGAATCACACAATATGAACGAT
CCTAATTCTGCACAATCTCTTAAGTTCCAGCAAATATGTTTTATGTCTAACTCTAATAAC
AGATTATTAGCGTCAGGTACCCCTGTTAAAGCACTTGGTTCTGAATTAGTAAGTGCATTA
AGAGTATTAGACGATCTCTTCACCAATGAGGTTGAAGAACGTTTTAAGAAAGCATTTGGA
AAAGAGACACAGAAAGGATTAGATATTGTCCAACATCGCTTTGGGTTAATTGCGTACGTC
ATTGAAAAGAAAGATACTGAGGTTTTACCACCGAGGATAAAAGCGTATCGTATCAAGATA
CCAAATGGATCACAGTATACTCTTAATGCCATTAAACGTGATTGTGAAGCTTTTATTAGA
GAACGTGTAAAGTTCTATGCTGCGCGCCGACCAGAAGATGAGAAGTATTGGGCTAAACTA
ATGGCGATACACGAGAACTCTCTTAAGACCCAGGCGCAGAAAGATGGTTTCGCTCGCTAT
AAGAATTTGATTAAAATAATTCAGAAGAATCAAGACCCGCGATACATTGGTGAAGAAATT
AAAGAATCTAACCAATATGAAAAAATGTTTATAGAGCCAACTTTACCTCGTAATGAAATA
GCTGCTTTTAGAGATATTAAATCAGTTATAAAATACGTTATGCTAAAGATACAAGGTGAA
TGTTTGGGAAGGGTTTTAGGTGCTAAACGCATTCAGTGCCATGTGGACATGGTACCGTAT
ATTGACTGGGTTGGTATCACTGAATCAACTATGAAGAAAACGATTGCATTTACATCGTTT
GTTGAGGTTGTTGATACTGTTGATAAGTACACCAATAAATTAGGTATGCATCCGGCGGTG
GTGTATGGTAAAACCAATGACAATTTACCTCAAATAATCAGTAGATTTGAGAAAGACCCT
AAATTAAATCCATTGGTAGCTACATATGCTTCCCTTTCAACAGCGGTCCGTATGACGATG
GCTGATACTATGGTGACAATTAATTCACCGTTTAGGCATTACATTCTAGAACAAGCTATT
GCTCGAATTTACCGTAAAGGTCAGGATTCACAAACAGTCGTATATCAATGTACTCTCGAT
ACAGAAGATGAACCGAATATCTCAACAAGGTCAGATGACATTCTAAAATGGAGTCAGGCA
ATGGTTGAAGCTATTATGGGTATTAAATCTCCATTTGAGATCACAGAGTCATTAGAAAGT
TATGTTGATAAAAATAATAATTTTGATGAAAATAAAATCATTTATCAGATGCTAAAAGAA
TCATTTGAGAAGTATGATATCGCAATTGATTTTAATGACTTTGATACACGTATGTATAAA
CCATATCAGCCCGCATATATGCGGTTTTAAAGGAGACAGAAATGTCTGATCCACATTCAT
CTAATACTGGTGAAGATCTTATTCTTGGGCCTGAAGCCACCAGTGAAGAAATGTATAATG
CGATTTTAAGGTGGGTTGACAAAGTAACAACCATCCTAGAATCTGGTGGTGGCCCTGGTC
CAGGTCCTAGTACTGGTTTAAAACCAATTCTATTTCAAACTGGTACATTAAACCCAGGAG
ATGTAGTTACTTATGATCTTTCCAAAGTTATTACTTCTATGGGTGATTATACTCTGGCTA
CAGCCATCGTTGATCTCAAGATTATCGACCCTGAAACTACTAGTCCTACTAGTGGTAAGC
TTATTAGCGCTTCTGCTAGTATTGGTTGGGATCTCGATTCATCTACAGGGATTTTAAATA
TCCGTGGGTATAGCCTGTCTGTACCAATCACTTATTATATTCGAGTTGACTCACCTATTA
AGACGGCTGATATTCCCGTGTATACCCCGTAAGGAGTGAGAAATGCCTTCTATTGTCCCG
TCGCAAGTGCCGGGTCCTGTGGCACTAAACTCTAAAGTTAAATTTAACTTTAGTTCAGAT
ATTCAGAAGGTGTATTATACAGTGAGCCCGGAGTTACCTCCGGTGCTCTCTGAATACATT
GCTTATGATGGTGACCCTGCAACAGGTGGTAGTCCATTTATTGCTGTTGTGCAAGATGGC
ACTGGTAATGTTCTTTATGATTGTAGTTTTCCAAAATTCTATAATTTAAACTATGATACT
TACCAAATTCCGGCTAATACAACAAACCCTCAAGATCTATGGGGATGGGTAAGATATTTT
TATAATGCTATTGGGTTTATTAGGAATGGTAGTAAGTACGAATCAACTGGAAAGAAATTC
TTAGTAATCACAGATAGTAATCCTAGTGAACCTTATTCAGTCCTCAATACGTCAGGTAAT
GGTTTTAAAAAGATACTAGATACGGCTGCTCGTGTACAAGATTCTACCTATGATGTTTAT
TACCCAGAAATGATGGGTGGCACAGTAGACCTTACTTTAACAGATCTTAATAAGTATTAT
GCATGTATTCTATTCTCTACTAAAGATCATAATACTGGTGCATTAACTCCTGAAAGAGTG
AGTCAGGCGACTATTGATAATCTAGCTACTTGGCGTAAATCGGGTAATGGTATCTTTGTT
ATAACTGATTCTGCTTCAAGAGATTACACTAATATTGAAGATGCTAAAGAAAATAATGGT
AGTTTTGCATTTACCGCTAATAGACTAGCCTTTAATTTTGGTTCTTATTTCTCTGGTGTT
GTTGATAGGTCAGATGTTCAATATACTGTAAATCAGATGCTAAGCTGGTCACCAAAAGCT
GCATCATCTGGTTTGTTTACAGGTATGAATAAATCTATTGGTAATGGTCAAGTAACTGAC
TTTGTTATCAAAGGTGATTCAAGTGAAAGTGAGATTAAGCTTGTTACTTTCCCAGCGTAT
AACAAAGAAGATATTCCTGAATTTACCTTCGATCATACTGGCGAGTACATTTACAATTTT
CTTTGTACGACATCTGAAAATCCTAATAAACCGTATGCTATTTCATTTAGATTCCAAGTT
GGTGTAGATGATGGTATCTTTGTTGCACGTGATAATGGCCAAGCTATTGATACTACTCAG
ATACTAACAGCTAAGCGTTATTTTGATCTTAATCTAGGTTACTCAAATGGTACTGACCAT
CAGGATTATCTAGGCGATATTCAAATAAATGGCTATTTAGTTGGATCATTCTTATTTAGT
CAGGGCATTACTCAGTGGGATCTGATAACTCATGGATCAACTCTTTTAATCCATGATAAT
GATCAGATACGTATTAACGTACGTGTACCTTATACTTACACAATGCAGCATCAAGTGGTA
CTTAGTTCAAAACCTGCTTCTCTTTATAATGAAGCATCCCTTATAGGTACAATGGCATGG
GATGATTATAAAGGGATAAGTAAAGCTAAAATTTATGAATATATTAATCTTAGCATGACC
AAGTATATGTATCGTGTAGCGAATGCATATAAGAATAACATTAATATAAAACCAATAGTA
TTGACTGCATTACGCCGTACATTCCTTGGTGAGACATTAAATACAGCTAATGTATATGTT
TATGATAATAACTCCACATGGGGTAAACTATCACAACGTGTCCAGAACGGACGACATGGT
GATATTGTTATCTTTGCAGATACTTCTAAAGTTGTCCAGTATGATGAACTGGCTTCAACA
CCTGGCTGGTATGTAATGAAAGATCCAGGATTAGCTGATGTAACAGTAGCTAAACTAGTT
CCATTCTTTGGAAATAACCGAGCTATTAAAAATAGGTTAAAGACTGGTGACATTCTTGAT
GAGACCACTGGTCAACCAATTTCTTTCCATGCTAATTGGAAAATAGAAAATGATAAACTT
GTAAAAGTTTAACGGCATAAGCCCTCCCGCAATGGGAGGGCATTATGTTGTTTTAACCAC
GCGTTATATTAAATAACCGTCTTTTCTGATCAAGTAATAAAATAGCGCTTGTGGTAATTT
TAATGTTAAACCTACGTTTAGCTAAACTCTGAGCTACTAGTTCAATAGCATCAACTAGAT
CGATTGATTCTATATCACCTGTATTTTTAGATTTACTAATTGGTTTACTAAGAATTTTCC
ATCCCTTAGTAAAATTACTAGCTGATGGCTTTTGACCTATCTTGCTACGAAAAAATAGTC
CAACTAATTTACTAGCCAGTTCACGTGGATCATTAAAATCTGCTTCTGGTGAATGTAAGG
TAATATTACTGTTTGCCAGGAATTCATGGTTCTCCGCACGATCCTCGATAGTTTTAGCAC
GTGGATCAAAATCCCAACAATTAACAGTTATATTTACGTCATTACCATTACGACGTAGAT
CAATACATCTAACGAAATCAGTCATGTCAAACTTACGTTCAACATCACGCATGCCATTAG
CAATCTTCTCTAATTCATCGTTATCAGTAATACCAACTTGCTTAGTAATATCAAAAGTCA
AATCGGCTAAAACGGATTGAGGAAAACTGGTTGCGATCAGCAAACGCAGTACGATTTCTG
GACGACTTTTTCTATATTTGTCGCCTGACATCTTCTCAAAGGTGTTAGTCATGATGGCTA
TCCCAAAAAGGTTAGTTCACACCATTTAAACGCTCCCAGTCATCTAGAGTTGCACGAGCA
TCAAATAGTGCGTTGTGAAGCATTTTAGAACCTTTTCCACTTAGCTCATGATTTAATTCA
AATATAGTTGGTAACTCAACAACTTGTTTACCTTTACCCATATCCATAGCTTTACCAGTG
TAAACCATATCGTTAACATGATTAGCTATAAAATGTAATCGAGCAAACTGACCAAGAAAC
TTGGCTAGTCTATCATGGAATTCTTCTAAAGAGATTGGTTCTTTTTGAAGAATAGGAATA
ACGTTTTCTTTAACCCATTCATCTTCAATATTGTCATATGGTAGAACTTCGTAGAATTCA
CAACGTCGATCTTCACTAACAATACCGAGTGAAATAAGAGTACCAGTAGGCGTATCAAAT
TCTGTATCAACAAAAAGTAACATAGTAGTTTTCCTTGTACGAAAGTCTTATCATAACAAT
AATGCACTTACGTCAAAAAGAGAGAACCATCATGGTTCTCTCCTATAAAAGTATTAGACT
TTTAGTCCACGAATATTGAAGATTATTTGACAAGCTGCAATTATTCTATTTCTAGAAATC
TTTACAGGAATTTTATAGTGATTGAGTACAGCTTTAGGATTCTCAACACTCATCAATTCT
TTAAGTAATTCGTCGCTAATTTTATTATGTGTTAGGACAAAGGTTGCGACATCGTGGTCC
CAGCATCGTTTTATTGCACTACAAGCACGAATATACTTCTCGGCTAATTCGGGGCTAAAA
TCTAATGCTTCTAGCCATTTCTTACGACCAGCAGTGTAAATCACTCGTGTACGAGTATTC
GATGGGACAGCTACAATATAACCTGTACCTAGTGTCCTACGCTCTTCAATGGGTCTGATG
TTCCAATCACCAATCATCCTAATTAATGTAGATAGATCATCATTAGCACCATAGTAACCA
ATAGGTAAACCTAAACTCACTTGTGTATCTATCTGGTCTACAGTTTGATGTGTAATAACC
ATTGTAGTTCTCTTCCCAATAGGGTTTTATAAACTTCCACAAGACTAAACTCCATTATAG
CCCTATCAGATTGGGTAATAAAACTTAACAAAAAAAATATTAAACATAAACCCTCTCCCG
AAGGAGAGGGTAATATATAACTTTAGCCGTACATTACAAAAGGTACTTCATAAACAATTA
GATAAGTATACATCAGTTTAAAACCAATGTGTAACCACTGATCTTGATTAAATGTAATTT
TATTATCACATTTTAAATAATCAATAATGCAATGAGCAATTGTTTCAATAAGTCCTAAAA
CTAATGAACCAGTGATGAAATAAACTGCACCGCCATGCATAAAACTATGCGCGGGTAATA
CCCATTTCCAATATAGAGAACCTAGTTCAGTGGTATGATCTTTAGCCTTTGCAATAAAAT
CATTTTGCAATGAAAAATCAAAGATGAAGTGTGCTACAATTAAACCAAAGAATAATGCCA
TTGAGCTGACACTGATTAATTCCATTTTATTTCCGCCTAGAAAGGTATTTCATCTTCAAC
AATGTGTGAATAGTCGATATTATGTTTTTTACATAATTCTTTAAACTTATTTAATTTAGT
TTCAGCTTCTAATAGATCAGAATGTAACTTTGTCTTATAGTTTTTACCGAAGTCAGCTGG
GTTGAATACGGAATTGTAATAAGCGTGTATCTCAGAATATTCTTTCTTTATAAAATCAAT
AAATTTTGGATCATTGCCATCACCAAGATAACTAATTAAATGGCCTAGATCATAATTCTG
TCGCCTAGCTGCTTTTAATATAAGTTCATTAATCTCAATTAATTCAGACGTATTACACCG
CCTTCTTTTAAAATATCGGATAACTTTATTAAATAGCATATTAAGTAATCCCTAGCCATT
GTAAATATCCACTATCACTTAATCCACGATAATAATCATCTTTATTTTTACGTAAATAAC
AATAAACTATTTTACCCATTAATTGATAACTAGTTTCATTATCAAAGGTAAATTCTTTTT
CTAATCTATTATTACTATCCACTAATGAGATTACTACATTACCGTAACGTACTGTTAATT
TCACAGTTCTAGTGGAATCAGCATCATGAAAGATATAAGTCAACCCATAATTCAATTCAT
TATTTAATACTTGAATACTAGTAGACCTATCAGATTTAATCTGAAGTGGATGTTCGACAA
TCGCCTTGAATGCTTTATTGCATAAGAGAATGTTTTGTATTTTTAATGAATCCATGACAT
TACCTGATTTAGTATTTATAATTTACCTATATCGAATTAGAATTAGGTAATAGTGGGTTG
ACTCAGTAGTCTTCAATCTAATCAGATCAGTGTTTAGACTGAGATTGAGTATTAAGCTGT
AGTGACACTTTAATCAATGACGTATTATGGATTCCCGGCAAGCCAGTTTACTGCACATGT
TGCTGCTTCATCAAATTGATTCAATGAGAATTTGGTTATCCGAACAGCCTGTGCCATATT
AAGAACTTTAAATTCACTATCACCAGACTCAATATAAGATATCATGGCATGATGATCATT
ACTAGTATCATAAACTGCAATGCGGATATCCTTGCCCCTTTCAACCACATATGCGATAGA
TCGTTTTGTTACAGGGTTATCGATGACATTAACTGTTTCACGTAATGTAGCGTCAATCTG
GGATGCATTAATATGGGTACTAATAGCATTCAGCAAATAATGCTCAATCTGTCGCCCTGT
TTCAAGAGTTAGAGAACCCATTGTAATCTCCTTTTAGAATCCAATATAATCTAGGAACCC
ATGGTAATCGAATGTTCGTGTTGGGATACTTGTATCCGAAAAGAACGTATTTATTTGTTT
AGCTAATTCAGTATGGTGATGTGAATTATCTTTGTTAATCTTAGTAACCACTTTCTCATC
AAAGAAATAGGTTACGCACATATTTAGTGGTTTATCTATATTGATTTCAACAACTACCTT
ACGAACCGTATCCTCAGACAAGAGTGTACACTTAGCGCCCCACGGTGAAACCTCTATTTT
AATATCTACATGAGGCTTGAGGTTATCATGATATTTCGTGAGTTCCTTCAATACATCATC
CATCGTTTGGATGAGCTTTATATTGGTAATATTCATTATAACCCCTCATAAATATCCTTT
CGTTTTGGATTTATGCCTGCAACCTTGCCTTTACTATTATCGATGTAACAAAGGTATTCA
TCACAAATGGTAGTCTTTACAAAAGGAATCAATTGGTTTAGTTCCAATAGAGTGTACTCT
TTACCCAAATATTTACACATCTGGGTCAATGGACTACACATCACATTATAAATTCCCTTA
GTATTTTTTAGAGAGAGGTATTCGCCATCAATATCTCTATCATTATTTATCATGTAAATA
TCACGAATAGGATAATTATCTTTTAATGGTCCGTTACCAAACTTAACACCATGTACACCA
TTGGGTCCATTTAGTAGATAATAACCAGTTGGCACAGACCAATATGTGGTGCCTTTAAAT
CCTGAAAGTAAACCATACGGCTCATGATAATCAGAAGTAAAGCATTGTTCATCATTACAG
ATGACTTCTTGATACATCGAATAATTAACAAGAATCTGTTTAGTTGGATCACTTGGTTTA
ACTAATGGAATAACTGAATATTCTGGATAACTAGTCCATCCGCAACTACCAAGTCCAGAT
TGTTGGTCGAAGTTACATGGAGCAATTCGCGGAGTATGATTAGAGTAGAATAGTGGTACA
CCATAAAAAGCAAGTGGTTCGTAATTAACACTAGCTTTAGTACACATGCCGATACTAATC
AGAAATACCGCACAAAATACACGCATCGATAATTTAAACATCAGTGCATCCACCCTCGGT
TTCCTTAATTGCCAATTTTACTAATACATTAACCCATGCACCTTCATCGAGCAGCCAACC
AGCAGCGCCTGTACCATGCTCGGCAAATCCAAGACTATCTAGATAATGTAGCATGAATGT
AGTTGGACCAAACCCATATTTCTTATTACATTCATTACGTTTAGAAGTATTCCGACCTTG
GCGATATATATCCAATACATCATCAATATAACGCAACTCCGCTTCTGGATAACCACAGCC
ACACAGGTTAAATAATTTGCGTGTAATGGTATTGTATGTATCACTATATTCTTTTTCATT
ATAATCAATGAAAGAGCCTACCCGTTTGAATAGTTGCTTATCGGATGCTAAATCATCAAA
ATGACCAGGGTAATCTTGAGATGGAAGTGCACCTAGCGCCATTAGTATAATAACGCGCGG
GTCTTTACGTTCAACTGATTCAATGAGTACTTCAAAGTTAAGTTCTCGTTGCACCACATT
GTTATTGCTGTTGATATTATTCAGCAGAATGTCCATAGCTTCTTGACCATGCCAACTACG
GATGTTAGGTTGGTTATTAGTATAACCAGTAATGGTCAATTGGTTAGCTGTATTTGATTC
ACCAACAGTAACATGTAGGTAATAAGGACATTGTTCTAATTTCTTAGTAATGCCAGTTGA
AGATTTATTACCCTCAAGTAGTCCAATATCGCCATGACCGATATACAAACAGATACCTTC
AGTAGAAGGTTGAGTGGTGTAGTTAAAGTTCAAGGATTTAAACATACTCTTTCCTCTATT
ATAAATGATTAGCTATTTACCGTTTATATAAAGATTAAATGGACATTGACACATGAATTC
CGCATTTAGAATACTTTCGATATTGTAACTAATTGATTTATCAGTGATGATGTTGATATC
TAAATGCCATGTCTTAGTATTTTGAGTAACTAACCTGTATTCAATGTCAATACTAAAACC
TGGTGCATCAACTGGATCATCATCTCCTTCATTCTGATGGATAAAGTATGAAGATAAAGT
ATACCTGGCTTTAATATATTCATCTGGTTTTATAACTTTTAAAGGGGCATTGATATGTGA
AATAAAACCATGATAATTAAATTCTTTATCCCTAATATCGATAACTTTAGGAGTAACCGA
TACATAGGCTGGCCATTGTGGTAGATCTAGTTCACCATGTTTAGATTGGTCTTTATCGAT
AATGTGAAAAGCTCTAACAATTCTATTGAACACTTGATATACCTTTTAACAAAAAAATTA
TAAAGAAGAGTCCCCGTAATGGGGACTCTATTATATCGTATTTTATTTTTTATCGCGATT
AGATGGTGCTAGTTTATAAAACTTCCGGAATTTATTAATAGACAATGTAAATGAATCTTC
CCTACCATCCGGATAACCAAGATAAACATCTCGGCGAGAGAGTTTAATTAGCTTATGCGC
TGTAGACACGCCATTTCGTTCGACTAGAAAAGTACGACCAATATAATCTTCTAGGTTATC
TAGATTAGTTGCAGGGATGTATTTAGATTCTTTCAGAGTCAGGGTAGTGCTCATTTAATT
TCTTCACCAATTACGAAATAATAAATTAATTTTATTTCCAGAGTGTTTTACCAATTAGAT
CATTTTCTGCAATGAATGCATCTAATTGATGTCCATAGTAAAGTAAACTATGAATTTGTT
GATTTAGACGTTCTTTATTTGAACCTAAGAACATCTTGTGCTTTTCACTATCAGGTAGCT
GATCGTAAACTCGTTGGCAGTACTTCTGTGCCACAACCTTACCAGATGCAAGGAAACGAT
CATCACCTTCGAATAACTGAGGGATAATATTTTCATCAAGATACTTACTGATTTCAGCAT
CTAGATCAAAATTAATAGTGTCATCGGTTGTTGCGACAGTATCTTCTTGCTGTTCATCAT
TCCATGGTATTTCAGAACTTACACTATCATCAAACTGATCACTAGTTGTAGTTTTAGCTA
GTTGTTCATCAAATGATGATTGTTTATTTTTTTGTTTATCACGCAGGAAAATAATTGATT
CAACAATCTCATTAACAATGATGTCTAGATCGCGATTGGTATCTACAACAATTGAACTAG
GTTGATTAATTAAGCGTCCATAACCTTCATCTACTTTATTAAAGTAACTGGAACCCTTAG
ACTCATAATAGTCAAGACCTCGGTCGCGCATTTCAATTCGTTTTTTACGAACTTCTTCAG
GAGCAGTTAAAAAGAAAGTAGCCGGTTCTGGAATAGAATGTAGTGTTCCTTGAAGAATAT
CCATAAATAGTTTTGATAGGTATGGATTAGTTTCTTCAAAAGGTACTACATTAAGAGCAT
AAGTAGATGCAATGAAACGTTCAGTGATAACGATTTTATCATCTTTTAAAGCAGGTCGAA
TAATGTTTTCTACATTAAGCATCCTACCTGCACTATGCATCAATACATCGAATTCTGGCG
GACGAACTTCATCTTTAAATTCTGGCCAAATTAACATTTGTCGAATCTTTTCCGATGTAG
CTGTACCACCTGGTTCACGCATTGCTACTACATCGTAATTGCGCTTACGTAGTTCCTCAA
CAACCATCTTTGTAACAGTTGTTTTACCAACACCTTCAGTTCCTTCGATACATACAAATA
AACCATTCATTACTAATCCCTCATCTTAAGTTTAATTACTGCTTCAACATTATCTTCTAA
ATCTTGTTGCGATAATTCTAATGAAGCTCTATAACCTAAGCTATATAGAACATCAACTAA
TACATCAATGCTTAAAGCTGGTAAATTAAACATTTTAATATTCTCACTAAGTTCATCAGA
GATGCCTAGTTTAGTGAAAACATCATCAATCAATACTTTTTGAGAAATGGCTTTTTGACG
GGCAGTTTGTACAATTGCAATAACTAGATTATATTTGATATTGTCAGAATGTGATTTTTT
ATTATCACCATGAACTTCTTTAAATAAATTCTGATCCGCCATAATACTCAGTTCCTTAAA
TTATTAAAATGTACTATTTAGCCATTAAACTTTTTTTAATATAAAAAATGACCAGTGGGC
AATGCACCCACTGATCTTATTATATTGAAATAGCTAGTATATATTAGCTATTGACATCTT
CAAAGAAGCTGGCTACACTTTCAACAACGTCAGTCATAGCGCTACTCTTATATTTGGTAT
CTACAACTGTTACAAGAGCACCACTGCGATCAATGATACCAGAAACAGTTGCACCGCCTG
CATTATAAGAATAACCAAGCTCAGCAATGCTTTCATTTTCTTTCATGTACGGTGCGGCTT
TTTCAGCAAATACATAACTTACAGCCGCTGCTAGTTGACCTGCACCTTCATGTACCTGCG
CATACTGCTCGGCAGTAACGCCTGCTGGTTGAAGAACAACTTCTTCAAATAGACCGTCAG
GGATCTCAAATACCCCGGCATCAGACATGCTTACTTTTTCATTTAGGTTGTGGGTTTCGA
TAAACTTCTTGGTGGTTTCGATGGACATTTTAGTACTCTCTCTATTTACGGTGGTGGTTT
AATTTACATCTTTTATCACAAAGCAACTACCATCATTAAAGACAACCTTAATTAGATTAT
CAGTTAATGGTCGTTTATATACAACACATTTTACATCGGGGAGTTTAACTACTTCTTTTA
AAGCATCTGGCATACCAATTAGATTAATGGTAGTTAATACTTTATTCAGTGGGCCAAGAT
GTGAAGTCAGTTCTTGCGCTGACTTAATCAAATCTACCATTGTTTTGTCTTTATCTACCA
TGCTAGTTACTCGATTTGATATATGAAGTACATACGTCACCCAGGTCTTCACCAGAGTCG
ATCATCTCACGAGCATCACCGATGGGAGTACCAGGGTGCTCACGCTCGGTTGCTTTAGCC
ACGTGTTCAATTGCAACTGGAGACCATCGAGCGGCAACAGCCTCACATACACCGATCGAA
TTCACTCTACGGATCATATGTGCACCAGTATCAATTACACTGAAACTGAGTTCACTAATA
ATTTCCACTCCTTTTAGTTTATCGATACTATCAATAACCTGCGTACTAATTAGATAATTA
GCTGTATCGTTATTATACGCAAGCACTAGTTTGAAACTCATTAGGGAGTTATCTTTATCG
GTGTCCCCAGCGATTAACTCATGATTTGAAGTGAGTGTATAGATACAACTACTCTCCTGA
GCAGTAATTGGTGATCCTGGTGAAGAACTATATACACTAGGTGCATTAGCTGTATTTAGT
GCAAACTTATTTACAGTGGAATTAGCGCCAGGAACTTTATAACCAATAGGTCTGGCAGTG
AAAATTACAACGCTCATTGTGATTCCTCTTTACTAGATAGATGGTTTCCTAATTACACAC
TAGTAATATAAATCTCAAATTTTTTTTAATACGAAATAAAGCCTCCCCGAAGGGAGGCAT
TTATGTCAATTACCACTATAGGTACCTTCAGTACCTTGTTCTTTACGTCTTACGAGGCGT
TGATGAATTGCACTAAGAGCACGGTCCATTGAATTAATCACTACCTGATTCTCTTCACAT
GCAAATGGGCCTGCTTGATAACCTTCAAATAAATCTTTAACGATTTTAAGTAGACAGTCA
GCAGTTAGGCCATTTACACCAACTTCTGGAACAAGTCCATTCTGCATTAGGATAACACAT
TCAGTCAGATTATCACCAGGCATAGCAGCTGGGTTAGTCTGAGCATTATAACCAGTTACC
ACATAACGTTTAGTTGCACCTGATTTCTGATCAATACCACTTGCTTCAATATTTAGACCA
TTTTCATCAAAATGTTCATGGCCATAAATACCTTGCATAGTTACCCATCCACCATCTTCT
TGATAAATCCAATCTACCCCATTTACATGATAAGTATCACCTGGTTCAGGGTCAGTTGGA
AGATCTTTAGTAGTGGTTAATGCACGGATACGCGCATATTCTTCAATTGCAATTTGTACA
TCAGTTGCAGTAATTTTCTTCTCAGGTTCAGTAGCTTCATCTTGTTGAGACTTATCGCTA
CCTTCCTTTTGATCATTATCCATATCGGAAGAATCTACTGTACTTTTAGCTTCTTTACTT
TCTAAGGAAGCTAACCACTCAAGATTAGGTTTAGACATACTTACCTCAATAAATGAAACC
ACCAATGTAAAGTGACACTACTACATCGGCAACCCGAGGGTCTTGTGAATATTTAGATTT
GACTAGTTTAATAAGTTCGGCTCGATTTTTAATGAAGCCTAGTTTCTTATTATCAATTTC
TTCAGCTTTAATTAGGAAGTCATCTAACTTCTTCTTACTACCCAGTAAATTAATCACCAT
TTGATCAATAACTGCATTAGAATTTGATAGTTTAGTTAATTCTTGTTCAAAAACTTCCTT
ACGTCCTTCTGATCCAATTAAAGCTAGACCGATCTTTTTAAGTAGAGACATGTTAATTAC
CTATTACATTGGTTTAAGAATGGTTACAATAAATAACTATTCCAAGTATTTAGAAGCGAC
CAGTGGTGTTAGCAAATGCGGATAGTGCACGTGCTGGTTCACCATCAAGAACCCAAGATA
AGAAGTTAGATGGGCTTCTCCAGTTCTTAAAGGATTGCGTAGCTCGTGTTATATTTAGGT
TCATACGTTTTCCAACATAATAGAAATCAGACCATGACTGAGATGTCAATGTGGCTGTAT
AATCTTGGAATAAACTTTGTTCATCCCATACTGAACCATCAGTTAATGCTAGACCTACAC
CAATACCACGATCACCATCTGCACCAATCCCTTCTGCAACTGCTTCACCAATTGTTGCAC
CAGCTGCTTTAAAAGCAGTGTCAATAATCGAACCGTCAGCAAAACCTGCCTTTAGTGGGA
TGTGCATTATCTTAGATAGATCTTTAACGTTTATTTGAACTTCACAGTTAAGCATGTGAT
TTTCAGCATTCCAACCAATATTACCAGTACCCCTTCTAATGGTAATTGAATCAACAATAC
CTAGTTGACGCTGTACCCGTCCTTGATGGAATATCTGACAAATAAATGGTGATGTATATG
CTGACCTACCAGCTGAACGAGGAAGCCCCATCGGTAAGATCATGGCTAATGGGATAAAAA
TATCCAAGAAGCGCGACATCACATTGCCATAAGGACATACTAAAGGAATTGTGTAAGATG
CTGTTGGCAATGAAGCAACTGAATTTTCCCAATGCTCGGGTACATCAACAAATGCGGAAC
CCGCTAATGTAGCCAACCCAGCCATGTTAACTGAATCTAAAGCACCACCAATGAAATCCA
TTACGCCTCTAGTAACATTACCCATCATTTCAGTAACATTACCTTGCATAAAGTTAAATT
GAGCAGCTCGTCCTTCTTGTACTTTACTATTTAAAGTTTGAGCTACACCAATGTCTGTTG
TGGTATTGGTAAATGATTCAGATACTTCTCCATTATGTTCAACCCGGAAAGTTACAAATT
GTGAACCATCCCGCTGAGCGCCAACTACGAAATCATAAACATTTTTCAACTTAGACCATG
ATGAAAAGTTTTCAGAATCAATTACACCAGCACCAGGTTTTCTATCAACTTCAATATAAC
TTAATAGATAATCCCGCGCAGAAACTTTTGAGTCGCCATTATCTTTAAAAGAGCTACGAA
GATAGTTATCTACCACATTATGTAAATCATCTTGGTTGCGTGCTTTCTCAACCAATGTAT
CCCATATCTTTTTGGCTTCATCGGATTTACGCTGTGTTCTACCTGATAAAGACATTATAT
CAAAAGAACCATCTTCCCTTATTAATCCAGGGAACATTCGATGGAAGTTAGCTAGATCGG
CAGGGGTTACTGTTTGACCAGGTATTTCTAGGTCTTTCTGAATACCATCGAAAACACGTG
GTCTAATACCTAAATTAATAGCAAATTCGTTAGCTAATGTATTCACAGCTGACCAGTAAG
CATGCATAGTCGGTTTAAAATAATACCATTTAGAAGGCTGTGTTTTGGCTAGAAAGTTAA
TAACCCTATTTGCTGCCGTGACTCCAATGATAAATGGCTGTAATGGCAATGATACTAAAT
AACCGGCAGCATTACCTAACTCGTACCAAGCTCTTGATGTTGTACCAGAGTTAGCGAATT
GTGCAGCTGACCGATCATAGAAGTTTGTAAAGAAACTAGCCCAACTACTAAAACGAGGAA
CACCGAATGACATATGGATTAATTGGGCTGTGTCGTCATGCGCCTCACTATAATATCGAC
CCATTCCTAAATGTCGTTCATCATAAGTCCTGCCAATTCCTGGTAAGCGTATATCTGCAT
AACGGGTATACTGGGGTAATGGATTAATTGCCCAGTTACCGCCAGGAGATGTGTTAGTGA
ATTTAAAAGCAGCTGAGGCAGCATTACGTCTTTTTGATCTTGAACCAAAACCAACTTGGC
GTTCATCTGGAGTAGGCAATAAAAATGCTTGCCTTAACCAAATTGGTTTACTATCTGTCG
CCATAGGAATCTCCTTGACAAAATAAGGAACCTCCCAATAGGGAGGCTCCTTTAATAGAT
TAACGTAGTCTAATTGGCAATGGACGGTTATCCACTACCCGACCAGGACGTTGAGAGGGA
GCTATAGTATTAGCTTGCTCTTGTCCAGTTTGTGCAGATGGATTAGCCGCTTTGTTTTTG
ATGTAATTAAGCATATCGGCATGGAATTGCTTCAGTACACTTAATTGCTCACTTGCTATA
TCTAACATGGCTTTATCTTGACTGGTAACTACTTTGCGCTGTTCCTCTATTTGAGAGGCG
CGCACTGTAGCCTGTGTTGCCGCACCGGTATCTACAACTGGCTGTTGACCAGAAGACACA
TCAGTCGCAGATGAAGGTGCACCAGGTAGCATCATTGATGGAGCATTCGTCCTTAATGGT
GATGCCATACCAGACATTATCTTCCCAAGTCCAATTTTATTATTAGAACTATCTGTTATT
GATGTATCAGCAGCACCACCGATAAGCTCCTTATCAGTAGCCATATCTTTTTGTTTAGCA
GTTGCTGCATCATTCATCAACTCTTCTGTTGATTTAGGTTTACCAAGTGATTGACTATTG
GCATTCTTACCGCCGCCAGTTCTAAACTTAGCAACCTTATTCTCCATGACCTGATAGATT
TCACCTAAGGTTCTAGCGCTACCATCCGTTTTAAAGAAGATAGATCTATTAGCCCTAGCT
TGGTCAGGGAATATCTCAGCACCAATAGAGTTCTGGTCACGTGTAAGGAACTTACGTGCA
CCAGCTGGACCCATGAAGTGCGCTAGATAAAGATCCACATCAGTGGGTGCCCTACCTAAT
GCATTTTCTAAATACTCATAATTGTCACGAAGGAACATCGCGCCCATAAGAGCATTTATA
CGTGGGTCAAACCGTAGTGAACCATTTTCATCATCAGCAGGTATACCAAACTTAGCTCCA
TATTTAGCAAGCATCCCTTTCCAGGTACTCTTAATAAATTGGAACCAGCCAGCTGCTGAT
GAGGTTGGTGCCTTGGCATTATAGTTGAATGATGATTCAATGCCACAGAAAATATTTAAT
AGTTCCACTGGTACACCAGTCATTTCTGATACAGCTTTAAATGTAGCTTGAGCTGCATCT
CTAGACTTATTAGATTGCGGCATTGGGATATTCTCCCATACACCACCATTCCCAGTTGCT
AATTGACCATAAGTCTGACCAGTTGGTGTTAATTCACCACCTGCCCCTAACTTATACTTA
GCCGTATAGTAAGTATCAGTTATCCCTTCTTGGGCTCTACCAATAACTTGACTAACTCGT
GCTGATGTATTTTCCATCCAGTTAGTTACTTTGGTCGTGGCACTATTGAATGTTGATTTA
ACATTATCAATAATCCGACTAGCAAAACTACGTCCTGAAGCTGCATCATTCTTACCTGCT
ATCTGTGCAGATTTAGATTTACCTGGAGCTTCAGCTACCTCAGTTTCAGCTTCTTTCTTT
ATCTCATCAAGACTTATTTTAGCTAGATCTTCTACTGAATCCATATCGCCAACAATATTA
AATATTGATGGTGCTTTCCATATTGAAATACCTTCATCATCAGTTGCACCCATTATCGCA
TTAGCAATCTGGACGCGCTGAGTTGCTGTTAATGTTTCTATTTGGCTAAGCGTTATCGAT
GTAGATACTGATCGAGCAGTAACAATAAATGCTTTAAATACAGGTAGGAATCGACGATAT
AACCAATCGACCACTTTAACACGGTCAGGGCCATCGCTAGTTGTAACCATACCAAATGCT
TGGCCAGTCCACTTAATTAATTCCTCTATATCACCTGTATAAGTAGCTTTACCATTAGCT
ACTCGCATTTTATCATTAACAAGACTTTCAATTGATAATAATCCATTTACAGCAGCTAAG
TCTAAACCTTGCATTCCATAAGCATGTACACGTAATGCTTGCAGTGAAGTTAATGAATCA
CTTGCATTTGCTTTAGGAATAATTGTGTCTACTGTTGTAGTTACCATTTTACGTATAGAT
TCAACTGGACTAACCGCTTTAATTGCTTTTCCAATAGTTGAATCCCTAAACCAATTAGTA
ATATCAGTTGACTTAGACTTAATCGTAGTAATAGTATCATTTATCACATCAGTAGTACTT
TCCTTAAAACTACTGGAAGTTGCTTTAGGTGCGACAGATGTTTTCTCTGTAGCGCTATAC
TTGTTTAATAATTCCTTTTCTTTCATCTGTATAGAAGCAACATCAATTGATAAGTCCTCT
TCACCAAATGGGTTATTAAGAACTAGATAAGGTGTATTACCTTCGTATGGGAATGATACT
GATCTAATGAGCTGTCCTTTTAGTTCATTAGGTACTTTACTATCAACCTCACTGATATCT
ACATCACTACGATTAATTTTGGATAGTCCTTTAAGCCATGCCTCAAAGATCGGTTTAAAT
CTGAAGTCGATCCAATTGGCTAAACGCATGATAGCTGGGCCATCATCAGCACCATATCCA
AATATCTTAATAATGTCACGTGCATTAAGAGCCCTTACATCAAAGGAGGCATCTCCACCA
CGTGTAGCTACACTAGATACAAGTTGTTCTAATTCAGTAACCTTGTTCATCTGATCAGTT
GTTGTAGCACCGTACTGCATCATTCGATAACTGATTAAGTCATCTTTGAATTTCTGACTT
CCTCTCCAACCTAATACAGGTTTTATTAAGAAATCTTTAAGTGCCCACATAATCGGATTA
CTGGTTACAATAGCTGCACCAGCTAATTTATCATTTGTTGATGCACCAGTACCACCAAAA
CTAGCATATGCAGCAGCTGTTGATAATGCTCCACCACCAATAGCTAAAGCACCTCTACCA
AGTATTTGGCGGGCAGCAGCCCATTTCCCACCTTTCTTACCACCAAGACCTAGGTCTTCC
AATAATCCTTCATCTTCCTCATCGCCTTTCTTCTTACCAAAGATATTCTTTAAGGTATCA
CCAAGTTTACCAAAAAGACCTTTTGATTCAGTGGTTTCTTCATCACTGTTATTTTTACCA
TTTAAAGTATCGCTAACACGTGATGTTAGATCTTGCCAGGAACCACGGCGAGGTTTATTA
GCATCTGGTTTCTGATTACGCAGTTCTTCCCATATACCTGCTAATAATTGATCAGTGGTG
GTATCTAGAGCTTTCTTATCTATCTGTGTTTCTACTTTACCAGTAAATGCACTAAAACGT
TTCTTGAAAGGTGATCCAAGTAATTTATATCCTAAACCCGCCATTCCTTTAGCAGTATCT
ACAGATTTCTTACCTAACCACTTCCAATAACGCATTGACATATTAGCATAACCACGGGCT
AGATTACCTAAACCAGTTCGGAATAAACCCTTATTCTTAGCAGCAGTATGCTTAGACCCA
TCAGAGTTAATAAGTTCAGGTATTTGTTCTTCATCAACAACAATGTTACCATCCCTATCT
AAAACTGGTCCATTAATCTCATCGAATGATTTTAATAGGTTACCAGCAGTTGTATAATAC
TCACCTGCTTTTAGTTTAACTTTCTGTAATACTGGTTTTAACATACCAGGTAAATATGCA
TCCCGATTACTTAGAATATCCTTACCTAATCCAATAGTAAAGTCTTTTGCTTTATTAGCA
ACTGTACCCATTGCATTAAAGATTTTCTGAGTAGTTGATCCACCACCTAGTACATTTCGT
TGTATCCAGTTAGATGCATCTGCTACATTCCTTCGCAGATTAAAACCATCTGACCGTATT
AAACCTTTAGCCAGTTCTTGAGCAGTTACGACTACATCACCAGCCCTGTTAATAATAGTA
CCTTTGGCATCTTTCAAATTAGTTATAATTTGATTAGTTGCTGTATCGATATACTCACCG
GCTAGTAGATCACGTCCAAGAATAACAGCTTCTTCACGGCCAGGTATATACCAATCTTTA
GCCATATCAGAAACATTATCTAAACCTTGAGTTAACCGTTCTCTAGCATCACCAGCGATC
TGTGCTGCTTTATCACGTAATTGACTAGCAATATCAGTTGCCTTGGTTAATAAATCACCA
CGTTGATTATAGAGACCAGTAGCTACTTCTTGAGCAGTTACTACAGTTATACCAGCTTGG
TTTTTAATTTCACCAGTAATATCAGATATTTTCTCTACTATCTTACCAGTATTAATATCA
ATTAAATTTCCATTTAAAAAATCCATTCCTTTGATAACTGGAGTAAAAGTATTAGCTTTG
GTATATAGATCAGCTATTGATTGATTGACTATATCAGAATATCCATTGAGCTGATTACTA
TAGTTAACAGTACGGCTATACATATCGCCAGCTTGGCCGTATAGTTGATCTCTTAAATCA
CCAAAGTTCATTGACGCTGGAATGTTTATATTCCTACGTAATTCGCTAAATTGATTAGGT
ACATTATCCCTTACATAATCCGTAGCTAGTGATACTTTTTGTTGTGCGTAATTATAGCCT
TTATTAGCTTGCTCTTGGGCCAGTTTAGAGGCACGAATAACTTGCTCTTGAGCTTGACGC
ATTTTTCGTTCAGTTTGTGACTGATACATTGGGCCAATAAAGTGCGGGTTATTCTGATCA
CCTAGTGGGGAGAGTTGCTCTGGACCACCATTATCAGAAGGTGGTGTATCACTATAACGA
TCAAATGGATTATCATTACCCCATCCTTCGTATTTTACTTCTGAACTCATTAGTTCCCAA
ATACGTGGGTAATTAATACGATCAATACCCTGCTCGGTTATGATAATACCCATTTCACGT
AATTGATCAGTTTTACCAGCATTGGTTAAACGCTCAATTTCTTTAATAGGATCACGTGAG
AAGTTTCTAACGTCTAAGAATAATGTAGATATTTCTTGACGCCTAGCAAAGTTTTCATTA
GTGCTTGCCATCCTACCATCAGCGCCAATATTGTATTGCCGTTTAATATATTCCGTTAAT
TCTTTAATCGCCTCACCGGACACTTTAGAACGATCATACCCACCAGCACGAGCATATGCT
TCGGGGTCAAAACGTTTATTCATATTGGCATCACGAATTAAACGTTCAGTTAGCGCTTTC
CTAGCATCTATTGATAGTTCTTTATTTGGGTCAAATGCTTCAAGAACATCGGTAAGAGCA
CCACTCACACCTCGTACAGTAGAACGACTAACTAGACGACGTTCTGTATTTGCAGCTGAA
TCCTCTACAGACATGAATGAACCAGTTGAGAGATCAAACACTTCACGTGTAATATCATCA
CGACCAGTCCGGATCATTCTTGTTTCTTGTAGTAACCTAGATAGATATCCAGGAATAGCT
TCAACTATAGTACGTTGTGTTAGTTGATTAAAGCCACTGTCCTGACCAATTGTTTGATAG
GAGCCAGTCTTTAATCGATCTTGTAATGTGAACTGTGGAATAAATGGAGCCATAATGTCA
CGAAGAATACCACGAGCACCATAAGTGTTTTGGTAATTATTCATGTATTCTTGTGTCCAG
GCAGGGAAGTTATCAAGTAAATAAGATGCTTGATTGTGTCGACCACCTAGATAGTTATTT
GAAAAACGTTCCACACCAGGACGAACCATTCTACCTAATACAGGTATAACTGAATTCCTT
GTCCAATCACCAAGATAAGAGCCAGCGAATTGACCAGCTATATTGTATCTATTCTCCCAT
GCATCCGCGCCAAACATGTTTCCAGCGCGAGCTGCATCTGCAAAGTTACGGATATTTTCA
TTCGCAAAGCGAGTAGCATTATTAGTTACGGCTGATCCAAAATTACCTAAGAAATTTGGT
AATGTTTGAATAGCGTTGTCAACAAAACTATTCGATACCCGTTGAGCTGCATTAAAGCCA
ACAAGATCCTTCATTGATGACTTTTTATGATCGGGAATCCCTGTGTTTCTAACAAGTGCT
GCAAAACCATTATTTAATAATTCCATGGTAGCTTCGGTTTGCTGTGCAATTTTCCGAAGA
TGCAGCATAGAACGAAACTGGATTTCTAATCCTTTCTTTTGAATACTGTAATTAACTTGT
TCATTATAACTAGCTAATTTAGATAAATTATCAACTGTTTGACCCATAGCCTTAGCCATC
CAGTCAAATCTATCAGCTGATACCTTATCGCGTATTCCACGTTCAGCTTGTCCAATAGCG
AATCGTTTAACTTCACCCTGTTCAGCTTTTATAGCTAAATCCCGTTGAACAAGAGTTACT
TGATCTATAGCAGATTTAAGTTCATCCTCGTCACGTTCAGCTTCCATGCGACGACGGATT
TCTTTATAATTTCTATTTGAATCTTCAGCGTACTTATAGTTTTCGATTTGACTTTCTAAT
CGATTATTAAGTCTGTCAAAAACAGAAGCTGGAACTTTATCTTTTAACTTAACTGCTAAA
TCTTGTGCTTCTCTTGTTAAAAAGAGTAAATCACTAGCACTCGTACGTTCTACAGAATCT
TTGACATCCCGAATAGTTGACATCGTATCTTCATATACACCGAACATACGTGAAAAGCCA
TCTGGTGCCGCAGACCGCATGAATGCTTTTAAAGCTTGTTGTGGTTTAACAGAGTCAAAG
AGTCCTTGTTTAATACCATTAAAAAATTCAGTGACAGGTGATCCCTTTTTAGCTTTCGGT
GTATCACCAAAAATATCATCGATATCACTTAAGTCAAGAATATTACCGGCTTGCCCTAAT
GTTGTACCGGTTGCACCAGTTTGACCTTTGGGCAATGTTACCTTCTTGGCCATATTAAAC
TCCATTGACGATTAGTAAAGTATCATATCGTTGTTTAACTTAGTATTACCAAAATAGGAA
AATAGAAAATGAAGAAGGCATTAGTTCCTTTTAATATTTCTTTACTTATTCCCACTAAAG
AACAATTAAAGTTCTTAGGTCAAGTAAAGAGTCATGAGATATTTGAAGGTTTAGGTGGTA
ACTTTCATGAAGATGGTTTGTTCTCTGTTTCCATTTTTGGAAGAGTTGGATCAACTGATC
GTGAGAGTTTATTTGGATATATCCATTTAGGTTTAGAACTTATCCATCCAGCTGTATATC
GAAACTTAATTAAATTAAAAGCTTTCTATGAAGAAATTATTCTTGGTAAGGCATTTGCAG
TATTTGACGAATCTATTAAAGATTTTATCCGCGCCGATGAACTAACTGGTAAAACTGGTT
ATACATTCTTTTTTGATAACTGGAAACGAATTGACTTTGGGTCCACTAAGTCAGGGGTTC
GGCTATCAAGGTTACAATTAATCGAAAAATATAAAGATAACTGTATTTTTAAAGACATGC
TTGTAACACCAGCCGCTTATCGTGATGCAGAGATTGATGAGAATAATCGTGTTGAGATGG
ATGAGATCAATGAACACTATAGGTATCTATTAAATCAGTCGTTTGGTGTACCTGATAGCT
TTGGACCAAATGATGACTTGAGTATCTATGATCGCAAACGAGTAGCAATGCAAAACACCG
TATTAGCTATTTATGAACATTATGAACGACTGCTATCCGGTAAACGTGGATACATTCAAT
CTAAATGGGCATCCCGTCGTATTGCCAATGGTACACGTAATGTCATTAGTTCGCTAGATA
CTAATGCTGCTGATCTTGATTCACCTAATCGACCAACATTCAAAGATGCCGTAGTTGGCT
TACATCAAGCAGCTCGTTCAGTTGCACCGCGTACAATCTTTGCACTAAGGCAAGGTGTAG
TTGGTGAAGTGTTTGATACAGTTACTAACACTGTACAGCTCATCAATAAGAAAACACTAC
GTTTAGAATGGGTCGATATCTCTAACGAAGATATGGATTTATGGGGAACTCCTGAAGGGC
AAGAACAAATCATCAATGAGCTAGATGTCATTGAAAAACGCTCACGTGCAATTGAAATTG
CTGATCATTATTTAGCACTTATCTACGTCGATGATAAACAAAATTTCAGGATATTCCGCA
ATATTGACGAATTACCTACAAACCTAAATAAGAAGTTTGTAAGACCTATCACATACGCTG
AGTTAGTTTACCTCAGTGGATTAAATATGTGGCGAACCAATTCAGCATTCGTTACACGTT
ACCCTGTAGAAAACTATAATAGTTCTATTCCTTGTAAAATGTACGTTAAAACTACTGTAG
TAGGTGAACTACGTTACCCATTAAATGAACAATGGGAACGGGACGATGCATTGGAACCAG
CGCTAGAGTTCCCAGTATTTGAACTAAATAAACCTGCACAGTGGCATGATTCTGTATCCA
TATCACCATCTATTCTAGCGCCATTAGGTGCGGATTAACTGTAGTACTGTATATCTGGTT
ATCCTATAGAAAATTAATAGGGTAATAACCATGTTAAATAAATCTATTGAAAATCTTTGA
TTTAAGAAAGTTCCCTACTCTGAACATGGTTATATTTTTAGAGATTATGCAGTAAAATAC
ATAAGTCCACATATTCCGAAATGAATATGTTTTACTCTTCTAATTGCTGGAAACTCCTGT
TAGACTTAAGTGCTACAATTATCACGAAAGTAGATAATGAATGCTTGACAAACTTAAGGT
AGGGCCAATCAGCAGCGAAGCCGCTAAGTTACTTAACTGTAATATGTGGAACGTTCAACG
ACTAGGGGGTTGCGCCCCGTAGGATCAAGTGATCCGAAATGGAGAGCACCTAAACAGATA
ATGCTGTAGGTGAAGATATAGTCTGGTCTTCTAGCGAGAGCTAGAGCTGCACGTAATGGT
GCGGGTATCAATTAACGACTGATACTGAACATAACGTTTGACGGGGACACTGTATCATTT
AACGCTGTTTATTCAGAGGAAGCTATCGCTGAAACAGATGCTTTCTTTAAGAAACGTATT
GCATATATACATGCCGATGGGCGACTGGCATTTAGTGTTGGTGTCCATACACTCAATTTA
ACTCTTCGGTACATGACTGGGGAACCCAAACAAAGGGATTAATCATGCGCTTAAATATTA
CACAGTTTCTGAAAAATTATAGTGTACGCGAATATGCTAAACTTCAGTCACCTAAGTTGC
ATGCTCTAAATAAACTTGATCTACCTTTCGAATCCATTTATCAGTTCTTCGATGGCAATA
ATGCAGTAATGGGTCCTAGTCAAACAGACCCATTATTCTCCAAACATCAAGGTAAAGTAT
ATATTGAGCATGTTACTGATATGCTTACATTTGAAGGTAATCCACGTAGGACTTCAAATA
TCCCAGCAACAATGATTCAAGAGTTTCGTCGCCAGAACCGTTTCTTTAAACCTTTACGCA
GTGATACCGGTTTTAAATTATCAAACCAGAACATTCTGGTAATGAACTATAATTTAATTA
ACCCTCAATGGAATTATATGGCTTCTTATAAAGCCACATGGTTCCGTTGGTCCAATGACA
TGCGTACATTCTGGAGTGGCGTAGCGGCTGCATGTGAACGCTTTCCTGGCTGGAACCAGT
TCATTGATGTACACTTACCTGAGTTAGTTCCACCTCCTTCTTCTTTTAATAAACTACGAG
GGGGTTTAACTCAAGATCTAGCTAACCAATTCAATACACCTGATATTCTAAATATATTTG
ATTTAGTTCGCTGGGTTTCTGATGATCGTGAAAGTTCTTTCATGAATGTCATCGATGCTA
AGTATTACGGTAACATTAATCTTTTATTCCGTGTACAAAGCTCATTCTTTGTTATTAACT
TAGGGATGCTTGATCAATGGCGTCGTGATCCAGAAATTAAAGATGATAAAGGTTATGATA
CCCAGCAAATAGCTAGGCGCCTTATTTCACTAGTATCAGCGATGGTTGAATATAACCAAG
GTAATACTTCATTAATTAAAGAGGATACGCCTATCTTTAATGAAGATATTATTGTTGATG
AAGAAACTCCGGTTAGCTATGAAGAAGGTGAAGTAGAAGAGGTTGAGGATGTTGAGTCTA
ATAACGTTGAAGAAGAAGTAACTGAAGTTGATGTTATCGATGATGTTCAGTTTGACCCAA
CTAACTTAACTAACTTAATTGATATTGGTGCAATTGAGGTAACATATACCCCACCACCAG
AAAGTGATCTTGAGGTAACAAAGCTTATCATTGAAAAAGATCCACTGGAATCTTCACCTT
CTCGCCAAATTAAAGAAGAGAAGGAACTCATCGTAGCAGATGTACCAGAAGATGAGTTAT
TTATCAATACCGAAGTTGATGAAGAAGATAAACTAATCGATGCCATTAGAGCTAAAGCAT
ACGACATGTATCGCGTGAATATGATTTCTGCGAATACATTCGAGCAAGCTCAAGAAGATT
CGATTATGTATCGTCGTCTACCTGATCCATTTACCGATCCTAATGATGAATCAGTAGAGC
CGATGACGATCGCTGAGGCGATGGAATATCATCCAGATGATCTTAAGATTCCAGAAGACA
CCACTTTTGAAGATAAGCCAACCATTGTTGATAAATCAATGTTGGGTAGTAAGTTAAAAG
CCATCCAACGCAAATATAACAAGGTCTTATTAAAGAAAGATATTTTAAACAGTGTTTTAT
CTGTTCAGAAACAAGGCGTATCAGTAACTAATTACAAAATTGAAACTGTGCGCGATTCTG
GTAACCATTACCAAATCCACAAAGTTACATTAAAGCCTATTCGTGGTAGAAGTTCACAAG
TGATGTTCCGTATCCCCGTCATTGATAATGACGGTAGGTTCATGTCTAATGGAGTAACAT
ATCGCCAACGTTTACAACGTGCTGATATTCCAATTCGTAAAGTTAATCCACGTAAAGTTG
CATTGACCTCTTATTACAATAAGACCTTTGTGACTAGATCTGAACGTGCTGAAAACAACT
TTGATAACTGGTTAATCAGAGCGATTACCAACCGTGCATTAGATGGAAGTGATATGTCTG
TACATTCAGTTACCTATGCTGAATTAGATCAATCCAACTATGTTCTACCAAGGGTGTACA
CTACTTTAGGAACAGCTTTCAAAGGCTTCCATAATTATCGTAATGTGTTCTATTTTAATT
TCAAAGATCGTAATGAATTCTTTGCAAAGCAAGGACTACATATCGAAGATTATGAAACTG
ATGATTTGATAATGGTGGGGTTCAATGGCACTGATGCTATCTTGTTAGATAAGAATTCCA
TCTTTTACATGAAAACAGGTAATGAGCTAGAACCTATTGGTACTATTACAGATATACTTG
GCTTAGATTTAACCAAAGCACCGTTAGAAGCTATTCTAATGTCAATTGGTGGTAAAGAGC
TACCATTAGGTTTCATTTTAGCTTATCACCATGGCTTAAACAATCTACTTAAGAAGTTGA
ATGTTAATTATCAGCGTCATCAGCGTGGCACTAGGATTGTTGTCAATAGTACGGATTATA
CATTAGCGTTCGCTGATGAGATCCTAGTATTTGATCGAGCTGATTATAAAGCAATGCTTG
TATTAAGTGGACTCAAACGTTATCATAAATCACTGCGGCGTTATTCTGTTTATGATTTCA
ATAAACGAGATGTTTACTTCAGGGTTCTTGAAGAAGCTGGATTAAGTAACAGATTTACAC
GTGATATTGACACATTCTTCTCAGCATGGGTCGATCCAATCACTGAGGGTCTACTTAAGG
AGATGGGTGAACCAACTACTTTTGAAGGATTGCTTTATCGGTCTGTCGAATTATTAACGA
ACGACTGGAGTCCAGCGGAAGTAGATGGTGCGTATATGCGCTATCGTGGTTATGAGCGCA
TGGCA

In another aspect, the invention provides an TIVP-H6 bacteriophage targeting Pseudomonas aeruginosa. In certain embodiments, TIVP-H6 binds a molecule of type IV pilus. In certain embodiments, the TIVP-H6 bacteriophage has a genome comprising a nucleic acid sequence comprising SEQ ID NO: 3.

TIVP-H6
(SEQ ID NO: 3)
GTGGCGGCCTGGACAGTGTGGTCGCTGCCACTTATGTCAAGAAGGTCATCGGGGTCGAGA
CCGAGCTCATTCACTTCTGCTACGGTAGCCGAGCGGAAGGCCCTGAGGTGATTGCTGTGC
AAGCTGCACCGGCCCAGCAGCCCTGGCAGCAGCCCGCAGCGGCCCAGCAGCCCTGGCAGC
AGCCCGCAGCGGCCCAGCCCTGGCAGCAAGCGCCCCAGCAGCAAGCTCCGGTTCAGCAAC
CTGCTCCGCAGCAAGCCGCTCCGGCACCCCAGCAGGCAGCGCCGGCGGGTTTCAATCCGC
AGACCGCCACCCCGCCCTGGGCTCAACCTCAGCAGTAAGCTGACCAGCATGGAGGGCGCA
TTCGAAAGAGTGCGCCCTCTTTTTCCGAGGAGATGTGATAATGGCAGATGATGCTGACCG
CGCACTCAAAGACGCGGAAGTGCTGGATGAGGCGCATATTCGGGAGATAAGCGCAAGAGC
GGCAAACATGCCCAAGGGTGTGCCTGGTATCTGCAAGCACTGTGACGAACACTTCGAACG
ACTGGTTGGCGGGCACTGCGGACGGTGCCGGGACTTGCTCAAGCTCCCCTAAGCCCTCTG
AGTCATATAAGGAGGCGTGATGCCTAATGTAATTCTCGCGACGCACACTGTCGCAAAGAT
CGACGCAATGGTGAAGGCGGATCAAGGGTCTGCCTTCCGTGAGTGGCTCGGAAAGGTGAT
CGGGCACATCGGTGACGCATACCGCACGGGTAACGAGGGGCACCGCTCGCACATGGGCGC
ATCGCTCATTGGCGGGGAATGTGCTCGCGCGATCTGGTATAACTTCCGCTGGGCAACGAA
ATCAAACTTCGAGGGCCGTCTGCTGCGCTTGTTCAATCGCGGACACCTGGAGGAAGCACG
TTTCATCGCAATGCTGCTGATGATCGGTTGCGACATCTACCAGCAAGACGAGAACGGGAA
CCAGTTCCGTATCTCTCACGCAGAAGGACATTTCGGTGGGAGTGGAGACGGTGTCGCTGT
GGGGATCCCAGACCTGCCTCCGGGCACTGCTGCACTGACCGAGTTCAAGACTCACAACGA
CAAATCCTTCACCGAGCTGAAGGCCAAGGGTGTGCGTGACGCCAAGTTCGAGCACTACGT
TCAGATGAACGTGTATATGCGCAAGATGGGCCTGGCTGTTGCGCTCTACCTTGCAGTGAA
TAAGAACACCGACGAACTCTACGGCGAGATCATTCCGCTCGACAGTGCTGTCGCTGATCA
GTTCCTGGACAGGGGCGACAAGATCGTTTGGATGTCCGAGCCCCCGAACAAGCTGAACGA
GTCGCCTGGATTCTTCAAGTGCAGATGGTGTGACCACAGGCCTGTCTGCCACCTCAAGGC
TGCTCCCGATAAGAACTGCCGGACGTGCGCCTACAGCGAACCCGTCGAAGGTGCCAAGTG
GATCTGCCGCTTGCATGATCAGCACATCGACAAGGAAGTGCAGCTGACCGGCTGCAAGGA
CTACACGCCCCGGAAGGTGTTCGGATGATCCAAGCTCGAAGCTATCAGGTCGAAGCGGTC
AGCGCGATCTATACTTACTTCGGGAACAACACGGGGAACCCTGTTGTCGCTATGCCCACG
GGTACGGGTAAGAGCGTCGTGATCGCGATGTTCCTGGAATCGGTGTTCAAGTATTACCCG
AACCAGCGGGTGATGATCCTTACTCACGTCAAGGAGCTGATTCAACAGAACTACGAGAAG
CTGATGGGCTTGTGGGCGTTCGCGCCCGCTGGTGTGTATAGCGCAGGCCTGAACCGACGG
GACGTGCATGCGCCCATCACCTTTGCTGGTATCGGTTCGGTCGCCAAGAAGTGGGCCATG
TTCGGCCATGTGGATCTTGTCATCATTGACGAAGCGCACCTCGTAAGCCCCAGCGAAGCG
ACCATGTATCAGACATTCCTGTCGGGCCTGATGAGCATCAACCCGAACTTGAAGGTGATC
GGTCTGACTGCTACACCGTGGAGGCTTGGGCATGGCAAGCTGACCGACCCTGTGAAGAAC
GACAAGGGTGAAGAAGTCCCAGGCCTATTCACGGACATCTGTTTCGACATCACAGGCATC
GAGGCGTTCAACCGACTCATCGCCGAGGGCTTCCTTGCGCCGCTGGTGCCCAAGAGCACC
GTGACGAAGCTGGAAGTAGATGGCGTTCATATGCGCGGCGGCGAGTTCATAGCGAGCGAG
CTGCAAACTGCTGTCGATAAGATGGACATCACAGTCGCAGCAGTCAAGGAGGCTCTAGAG
CTAGGTTGGAACCGGAATCACTGGCTGGTGTTCGCTGCCGGTGTGGAACATGCTATCCAC
ACCGCTGAGATCATGAATGACATGGGGATCCCGACTGTTGCGATTCACTCCAAGATGGGT
GACAAGGAACGCGACAATGCGATCAGGGACTTCAAGGCTGGGAAGTACCGCGCGGCAGTC
AATAACAACGTGCTGACCACAGGGTTCGACTTCCCTGCCATCGACCTGATCTTGTGTCTG
CGCCCGACAGCTTCGGCGGTGCTGTGGGTGCAGATGCTAGGACGAGGAACGCGCCCAAGC
CCTTGGACAGGGAAAGAGAATTGCCTGGTGCTTGACTTCGCGAACAACACACGCAGGCTC
GGACCGATCAATGATCCTGTGGTTCCTCGTCGTAAGGGTGAGAAGGGTGGAGACGCACCC
GTCAAGGAATGCCCGTGCTGCCGCACATGGGTTCATGCCAGCCTGCGCTGGTGTAATGGA
CTGATGCCCGACGGTTCGAACTGCACTTACGAATTCAAGTTCCAGACCAAGCTGAAGCAG
GGCGCCAGCACCGCGGAGCTGATCAAGGGTGACATGCCCGTCGTTGAAGTGTTCAAGGTG
GATCACATCACCTATATCGAGCACAAGAAGGACGGCAGGCCTCCAATGATGAAGGCCACC
TACTATTGCGGGTATCGGATGTTCGAAGAGTTCGTCTGTGTCGAACATACGAACTATGCA
GGCAAGAAGGCGCGCGACTGGTGGCGTGCGCGTAGCGACGAGCCTATGCCCAGCACTACG
GCGGAGGCGCTGGAGCGTGCTGACAGGGTACGCACGCCAACGCACCTGAGAGTCTGGATC
AACAAGAAGTATCCGGAAATCCTTGCGACGTGCTTTGATGGCACCGCGTTCGGCACTCAG
GAAGCAAGTGACGGTGACGAAGGCCCCAGCGTGCAGACGCACCACAGCGCACCGCTTGAG
GATCAACGTGCAAGCGAACCGACCAGCTATGCGGATCTGGACGATGATATTCCGTTCTAA
TCGATTTTTGGATGCCGATAAAAATCTTTTTTACGGGCAAGCCTGAAAAAGGGCTTGCAT
CTTTGTCCAGTTCGTCTATAGTACGATTCATGGATGCAGCGGTGCATCAAACAACGGTCG
CAAGACCATCAACCTCTAGGAGCTTAAAATGATCAACTTCAACGAAATGGGCAAAGTGGA
ACTCCGTCAGGCTTGCAAAGAAGCTGGCATCAAGAACTATGGCAAGATGAATAATGACGG
CATGCGTGCCGCGCTGGAAGCGCACTACGCAGAGGCCAAGGGTGCGGAAGAAGCTGCGCC
GGAAGCTGTGGTCACCGAGGAAGTGCAGGAAGAAGCACCTGTCAGCGCCCCGAGCGGTTT
GGCGACACTGGTTCAACAGATGATCGGTGCCAACGAGAAGAAGGAAGAGGAGCGCAGCGC
TCCCGCCGCTGCCAAGCGCACCAGCAGCGGCCTCAAGATTGAGAAGGCACGCGAAGAACG
CAACGGCATCAAGAAGCCCTCCGTGGGCGGTCAGTGCCGCGCTGTGTGGGACGCGCTGGA
CGACATGGTGGCAGCAGGCACCCAGCCCACCGCCAAGCAAGTGAAAGCGTTGGCGGAAGA
ACGCGGCTGGAATCCGAACAATGCGTCCATCGAGTTCTACCAGTGGCGCAAGTTCAACGG
CATCCGCGGCCGTCAGTAATAACAGCAACGAGGGGCGAAAGCCCCTCCGGAGGCATGTCA
TGATTCGGTTTCTGTTAGTGCTTGCAGCCATTATCCTTGTGCTCTATCTGATCTTCTCCG
AGGTGTTCGCTACCGCTGAGCGCTTCAAGCACAAGGCAGAATGCCGACAGCCGCTTGCGG
CATACATGCACGGAGGGGCGCCCTTCAGATGTGAGGTGAAAGATGTACGTTAGTCTCGAC
ATGCAGAACATGAGGATCGTCCATAAGCATTCGTCCGTGAATGCGGTGTGCGGCCTGGTG
CATATTGAACTCCCCGACGTCGCAGTGAATGTCTGTCCGATCGACATGACAGTCAAGCAC
AGGACCGACATGGAGATCAAGATGCTCTTCCGATCGTGCTTCCCAGGGCAAGCTGACCAC
ATGCCCGTCGCGGAGATGAAGTCCAAGATCCTCCAGTTCGCTGAGGAGTTTCCGGTCACA
GACTTGGACGAGCTTGAAGTCAAGCGCCAAGCTGACAGCATTCGGGATGGCGACAAGAAG
GCCTACAAGTATGTGAAGGGTTCGTTTCGTGCGTCTCGACCTGCTGAGCTGTTTGCCGAC
GCTACGGGCGACGCTGCGCGTGCTAGTGCTACCGTACCGGCCAGCAGTGCTGCGCGGCCT
GTGCGGCCCGCTGCCGCCCCGCGCGCTGCAACTGGTTCCCCAAGGGCCAGCGGTGTGCGC
GAGAAGATCTGGGCAGTCGCTGACCGCATGTGGGAGGAAGCTGGAAAACCGATCGAAAAG
AGCACAGTCCTGGCGCTGCGCAAGGATATCATGAACGCTTTGGAGCAGGATGGGGTGAAG
CGCACCAGTAGCTCCAATGAGTTGGGCAACTGGCAGAAAGCGAGGATCACGTAAGATATC
CGATTTGCCCGCTTGCACTCATGCTCCCGGAGTGTTACATTGCAATCCCGTCCATCAACG
GACTTTATTCACTTTCACTGGAGAAAGACCATGACCGAGAAGACCGCCGAACAGATCGCT
GCCGAGCAGGCCGAAGCCCAGCGCAAGGCTGACGAAAAAGCTGCCGAGAAGGCCCGCAAG
GAAGCCGAGAAGGCTGAAGCCAAGGCCAAGCGTGAAGCCGAAGCTGCCGAGAAAAAGGCT
GCCAAGGAAGCGCAGAAGGCTCAGGAAAAAGCCGACCGCGAGGCCAAGAAGGCTGCCGAG
AAGGCTGCGAAGGAACAGGCCAAGGCCGACGCCAAGGCTGCCCGCGAAGCCAACCGCATG
CCGGAGCAGAACGGTATCCGCCGTCCGAAGCCCGACACCCTCTGCGGCAAGGCCTGGGGC
ATCTTCGACAGCGTGTCGCAGAAGAACGGCGCTCCCGCGTCCATCGGCGAGTCCATGGAA
CTGGCCAAGGCCGACGGTCTGAACGAAGCCAACGTCCGTGCCGAATACGCCCGCTGGCGC
AAGTTCCACGGCATCACCGGCCGTATCGAGTCCCCGAAGGCCGAAACGACCTCCGCTGGC
GCCGAGCGGGCCTAACCTGCTACAATAAAGCGCCCTGGGCATCCGCCCAGCGGGAAACCT
GCAAAGGGGCAGAGATGCCCCTTTGCTATCTACCCCGGAGGTTCCATGAACTTACAACCA
ATTGAGAAGCGCACCAAGAGCGCTGACGGCCTGCTGTCCTTACACAGCATCTTTCACACC
ATCCAGGGCGAAGGCCCTTTCTGCGGAACCCCTTCCGTATTCGTTCGACTCGCAGGGTGC
AATCTCCAATGCCCCGCATGCGACACCGACTACACCCAAGGCCGGCGCGCTGCAAGCGCC
CAAGAGATCCTCGACAAGGTGCAGGAATACCAGTCGAGCGGCCTTGTGGTCATCACCGGA
GGCGAACCCTTTCGTCAGGACATCACACGACTCCTCAACGTACTGACCGACGCGGGCTTC
TACGTCCAGATCGAGACTAACGGTACGCTGGAACCTGTCGAGTACCCTTACAGCACCATG
CCCGACATTCGAACGGGCGTCTATGTGGTGTGCAGCCCCAAGGCGGGCAAGGTGCATCCT
CGCATCAACGACGTCGCATGCTGCTTCAAATACGTGATCGCACATGACAGCATCCATGAT
GATGACGGTCTGCCCTTGCGTGCTCTGGAACATCGCGCGAGTCCGTATGTGGCCCGACCG
CCCAAGAACTGGGCACGCCCAATCTACCTGCAACCGATGGACTCCAAGGATGAAGAGCTC
AACCGCCTCAACCTGCAAGCGGCCATCGACTCCTGCATGAAATTCGGTTACATCCTTCAA
CTACAGATCCACAAGATCATCAACATGGAGTGAGCTATGTGCGCAATCATTGGCGCCTTG
GTGTGGGGCTTGACCACACCTGAAGCACGGTCACAGGCCAACAGCCTGCTGAACCATATT
GTGGCACACAGCCATGAACGCGGTCGCGACGGTCGGGGCTTCCTGACGAACTACGGTGAC
GACATCGTTATCGAGAAGAGCACCGACCGCAAGGACGCGAAGGACTGGGCGCCTGTCGAG
TTCTTCAAGGGCGACGTGTCGTCCGCTACCTTTATCAGCAACCTGCGAGCCGAACCGACA
ACCGAGTACGTCGCTGACAAGTCCCAGGACGATCAGCAACCCTACAGCGCGGGGCACTGG
TCCATCGTGCATAACGGCACCATCGCCAACGATAAGGCGCTGCGCACTGGCAAGGTGCCC
ACCCGCATCGACAGCGCGGCGATCGCGGAAGTCCTGGACGCCAACCGCTGTGACGGTTCC
AATGCGTACCGGACCGCTCTTCACTTCATAGAAGGTGTGAAGAAACTCAAGGGCAGCTAT
GCGATCCTGGCGACACATAGCGAGCAGAAGAATCATCTGCTGGTGGCAGCAAACTACCGC
CCAATCTGGTTCGTCGTAACCGAAGTCGGCGTCTTCTTTGCAAGCGCGCGCCACTACTTT
CCCGACAACATGACTCCGCGCATGGTCGATCCGTATTCGGTCATGAGTTTCTACTTCAGC
GATCATCTTGAGATCCGCGGCGAGTCCCTGTACGACGTGGGCGCTCGTTCCAAGGCGCTC
GTCGTGTGTAGTGGCGGCCTGGACAGTGTGGTCGCTGCCACTTATGTCAAGAAGGTCATC
GGGGTCGAGACCGAGCTCATTCACTTCTGCTACGGTAGCCGAGCGGAAGGCCCTGAGGTG
ATTGCTGTGCAAGCTGCCGCTGACGCGCTGGGCGTGCCCTGCACCGTGTTCCCGCTCCCC
GTGTATAAGCCAAGCGACTCCCCGCTGCTGCAAGCTGACAGCAAGATCGCTGGCGGGGAG
GAAGGCGCCGAATTCGCTCACGAATGGGTGCCTGCGCGGAACCTTCTGCTGCTCAGTGTG
GCTACTGCCTACGCGGAAGCGAACGGATTCGACACTATCGTCCTCGGTAACAACCTCGAG
GAAGCTGGTGCGTATCCTGACAACGAACCCGAGTTCATTGCCAAGTTCAACGACCTGCTG
CCTTTCGCAGTTGGCGATGGAAAGCGCATGCGAGTCATGATGCCTGTGGGCAACCTGATG
AAGCATGAGATCGTGGCGCTGGGGCACCGCATTGGCGCACCGCTCGACAAGACCTGGAGC
TGCTACCGTGCGGGCGAGCAGCATTGCGGGACGTGCGGTCCGTGCTACATGCGACGCACC
GCGTTTGACATCAACGGCCTCGAAGAGGTCATTTCTTACAGTAACGACAAGGAGTAACCA
CATGCCCTGGCAAGCCAAACGATATCACGACATCAGCTGCGGACACCGCGTCTTCCAGCA
CGAGTCGAAGTGCGCGCATCTGCACGGCCACAACTATCGAGTCCATTTCACCTGCGAAGC
GGAAGAACTGGACAACATCGGCCGCGTGATCGATTTCAGCGACATGAAGTCTCGCCTGTG
CATGTGGCTGGAAGATAATTGGGATCACAAGACGCTCATCTGGGAGAACGACCCGTGGGC
CAAGGTGCTACCCGAAATCGACCCGACGATCGTGATCGTTCCCTTCAACCCTACCGCCGA
GAACATTGCTCAACATCTCGTTGAAGTGATCGGCCCGCAGCAGCTCGCAGGCACCGGCAT
CAAACTCGTACATTGTGACGTCGAAGAGACGCGCAAGTGCTCCGCTTCTTTTCACGCTCA
CTGAGGACACACCATGCAACTGACTCTGAATCATAACGACGTGCAAGCGCTCGCGCAGCG
GGCGGCACAGGCCATTCTGGCCTTCGGTGCTACCATTGGCCGCACCCTGCTGGCTTACCC
CGTACCGCGCGGCGGTGTGCCCGTTGCGTACCTGCTGCAAGGGCTCGCGGGGTTCAGCAT
CACCGACGACCCCGAGAAAGCCGATTTCTTCATCGACGATCTGATCGACAGCGGCAGCAC
GATGGAACACTGGTGCGACCAGTTCCCAGGCAAGCCCTTCTTCGTGCTCATCGACAAGAA
GACGGACGAAGAGTTCAAGGGCCGCTGGATCGTGTTCCCGTGGGAAGGCGACGCTGAGTC
AGGCATCGAGGACAACATCCGCCGTCTGCTCCAGTACGTCGGGGACGATCCTGCCCGCGA
AGGGCTGCTCGAAACTCCGCATCGCGTCGCAAAAGCGTGGCGTCACTGGTGCGGTGGCTA
CGGTAAGGATCCGAAGGCGATCCTGAAGGTGTTCGAAGACGGTGCTGAGAAGCACGACCA
GATGGTGACGGTGCGCGACATCCCCATCTATTCGCATTGCGAACACCATCTGGCGCCCAT
CTTCGGCACGGTGACGATCAGCTACATCCCGAACGGTCGTATCGTCGGGCTCAGCAAGCT
GTCCCGCCTGGCGGACATGTATGCACGCCGCCTGCAAGTGCAGGAACGGCTCACCGACCA
GATTGCGGACGCTCTGTTCGAGCACCTGGAAGCGAAGGGCGTCGGTGTGGTCATCAAGGC
TCGCCACATGTGCATGGAGTCGCGTGGCATCTGCCAGCAGGGACACCACACCGTGACGAC
TGCTCTTCGCGGTGCGATGAAGGATGAGCCCGACACCCGTGCCGAGTTTCTTCGGCTTGC
CCAGTAACAGCTAGTTCGCTATAATGTAGGGACGTCTTCGGATGTCCCTACATTTTGGAG
GTCTCATGAACATATTCATGGCGGCCGTTTATACGAACGGCTATATGGAAGGTCAGGCGC
GCCATGCCAAGCTGACGGAGCACGAGCAGAACATCTGCAAGAATCTTCCTCACATTCTTG
AGTCGTACCACTACGTCGGCAAACAGCGGTATGTCGATGACATGCGCCGCGATGGTGCTC
AAGTGTTCCTGGACTCGGGAGCGTTCTCTGCTTACACGCTGGGCGTCGAAATTAGCGTCG
CTGAGTATTGCGACTATATCAACCGCAACAGAGACATCATCCGCGTCGAAGATGGTGTCG
TGATGGCATCCGTGCTGGACGGCATCGGTGACCCATTGCAGACGTGGCGCAATCAGCTAG
AGATGGAGCAGCGCGGCGCCAAGCCCCTTCCCTGCTTCCACGCTGGGGAAGACGAGCGAT
ATCTTGAATGGTACGTCGCGAACTACGAATACATCACGCTCGGCGGCATGGTGGGCTCCA
GCACACAGCAGCTCTGTATCTGGCTGGACAGAATCTGGGACCGCTACCTCACCGACGGTA
GCGGCAATCCGAAGATCAAGGTGCATGGCTTCGGTATCACGGCCGTTCCAATCATGGAGC
GTTATCCGTGGTATTCCTGTGACTCGTCCTCTTGGATTCAGTCCGCTGCATTCGGTAGCA
TTATCACCCCAGGCTACAGCCAGAGCACGATGGCGCTTCCGATGAGTGTATCGGAGAAGT
CCCCCAGCAGGCATGACGCTGGCCAGCATGTGACCACACTGACACCCATCGAGCAGGATT
ACGTTCTGCAGATGCTCGAAAAGCACGGTTTCACGCTTGAGCGCCTTTCCACCGTGTATG
AGTCACGCGCCGCCTACAACCTGTGGGCGTTCGGGGTGATCAACACAATGATCAACGCTG
CGCACTCGAACACGTTCCGGGCGCGCATTCAGGAGCTCTTCTAATGCTTAAAGAACTCAA
GTTCGTGCAGGGCGCAGTCGCTAAGAAAGACCTCCTGCCCGCCATGACTCACTTTGCCAT
CGAGGGTGGGCATGTGCGCTCGTATAACGGCACGCTGGCGCTGTCCAGCCCCATCCCATT
TGACATTGACTGCAAGCCCAAGGCGGGCCCGCTGGTGCAGGCGATCGCGAACTGTAACGA
CACAGTGACGCTGAGCATGACCCCAGCGGGCAAGCTGCGCATTCAGAGCGGTAAGTTCCG
GGCGTTCGTGGATTGCATCGACGGTGAAACGCCACACGTCATGCCTGAAGGTGATCACGT
AGATCTTGACGGTGAGCAGCTTCTGAAGGCGTGCAAGACGCTTGCACCGTTCGTCGGAAA
CGATGCGTCCCGTCCTTGGACGAACGGCATCTTACTTCGGGGTCAGTCTGCGTTCGCCAC
GAATAACGTGTGCCTGGTGGAATACTGGATCGGTTCGCAGACACCCTTCACCGCCAACAT
TCCTATGGCGGCAATCAAAGAGATGATCCGTATCAACGAAGCGCCTACGCACGCACAGCG
CACCGACAACAGCATCACATTCCATTACACCGACGGGCGCTGGATCCGGAGCCAGCTGTA
TAGCACTGACTGGCCCGACCTGTCGAAAGTGCTCGACAGACAGTGCAACCCGAGTGACAT
GGACTCCCGTCTGTTCGAAGGGCTGGAAGTGATTAAGCCGTTCGCTGACAAGCTGGGACG
CGCGTTCTTCCACGGTGAAGCAGTCAAGACCCATATCGACGAAGGTGAAGGCGCATCGTT
CGACATGGTGGGCTTCCCGTTCGAGGGTGTGTATCAGATCGAGATGCTCAAGCTGCTGAA
AGATGTGGCACAGAAAATCGACTTCAGCACGTACCCGAGCCCCTGCCTGTTCTACGGTGA
CAGGCTCAGAGGTGCAATCGTAGGCATGCGCCTGTAACATGTAACCCAGCGCCACGGCCC
GCTTAATGGCCTTAGATGAGGATAGAAGATGCGAGCTGACGCTCTAGGACTGTTCTGGGA
GGATCGACCGCCACCTCCCAAAGAGAAGGCCGAGAAGGCCAAACGCACACCGCCGGACCC
TGTGTGGTTACGCCCCGACTACCTGCCGGGCCTTGAAGAAGCTCTACGCTTCCCCATGCT
GGTGATGAACGACGCGGAGGTGTTGCACGCTGCAATGACCCGCGACAGGCTGCTGTTCGA
CATCGAGTGTTATGAGAACTATTTCATCGCCTCGTTTCGATCGCTCGCTACAGGCAAAGC
GGTGTTTTTTGAGATGTACGATGGGCATCCGCTTGACGTCGGGAAACTGCGCTGGGTACT
GGAGAACACCACGACCGTCGGGTTCAACTCCCTGTCGTACGACTTACCGATCACATTCCT
TGCACTTGCGGGCAAGCCGAACGCATTGCTGAAGCACGCGACGAACCAGATCATTCTGGA
GGGTTGGCGCGGGTCCGACGTGATGAAGCAGTACAAGGTCAAGGGTGTTAAGATTGATCA
CATTGACCTGATCGAGGTGGCTCCGCTCCGCGCCAGCCTGAAGATCTACGGTGGCCGTCT
GCACGTTCCGCGAATGCAAGACTTGCCGTTCCACCCTGACACCGTACTCAGCCCTGAACA
GATTGCCTGCGTGCGCTGGTACAACATCAACAGCGACTTGACGAGCACCGCGTTCCTTCA
TGAGTGCTTGCGAGATCACCTAGATCTGCGCACTACGCTATCGAATGAGTCTGGCATTGA
CCTTCGCAGTAAGTCTGACGCGCAAATTGCTGAAGCTGTGATCGCCGAGGAGATGACCCG
TCGTATGGGCAGGCGCCCTGAGAAGCCCGTGATCGAGGTTGGCACCGCATACCGTTACAG
GGTGCCTCACTTTATTCGCTACCAGTCGCAGCTGATGAACTGGGCCCTGAGTGTCGTTGC
GAATGCGAACTTCCTTGTGGATGAGACTGGTTCCATCGGCATGCCTGAAGAGATTAAGGC
GCTCCAGCTTGATATCAACGGCAGCGTGTATCGCATGGGCATCGGCGGTCTGCACAGCAG
TGAAAGCACAGCAGCGCACCACAGCGACGAAAACTACATCCTGATCGACAAGGACGTGAC
GTCGTACTACCCGTTCATCATCTTGAACCTCGGCCTGTACCCGCATCACCTTGGCCCGAC
GTTCCTTCACGTGTATAAGAACATCGTCGATCGGCGTATTGCAGCCAAGGAACGGGGTGA
CAAGGTAACGGCTGACTCGCTCAAGATTACGATCAACGGGTCGTTCGGTAAGCTGGGCAG
CAAGTATTCGATCCTCTACTCCCCTGACCTGCTGATCCAGGTGACGGTGACTGGCCAGCT
GAGCCTGCTGATGCTGATCGAGCGGCTGGAACTGGCAGGCATCCATGTGGTTAGCGCGAA
CACTGACGGCATCGTGATTAAGTGCCCTCGCAAGGGGCAGCACATCATGGACCAGATCAT
CGCGCAATGGGAACAGGACACGGGCTTCCAAACTGAGGGAACCATTTACAAGTCGCTCTA
TTCCCGGGACGTGAATAACTACATCGCAGTCAAGGAAGACGGTTCTACGAAGTGCAAGGG
TGCCTATGCCAACCCGTGGGCCAGCAAGAAGAACATGGCCGAGCGCTTGCACAAGAACCC
GACGAATACGATCTGTGTCGAAGCTGTGGAGGCACTGCTGACGAAGAACGTACCGATCAT
GACCACAGTGCGCGGGTGCAAGGACATCACGAAGTTCGTGTCCGTGCGCAGCGTCAAGGG
TGGCGCGGTCAAGGTATGGGATCGAATTCCGCCTCCCGCGCATAGTTCGCAGGAGGAGCT
TCTGAAGCTGGCGGGGTTCGTACCGTTCTCGAAAGATAGCTGGGTCAGGGAAGGCGACAC
CATCGGACGCGCTGCAATGAACACCGAACGCGCGTACAAGGCGGCCTGGGACGAGCTGTC
CAAGCCTGGTCTGACCGAGTATCTGGGCAAGTCGATCCGCTGGTACTACGCGGTCGGGGT
GCAGGGTGAGATCGTCTATGCAACGAACGGGAACAAGGTGCCCCGCTCGGAAGGTGCTAA
GCCCATGATGGACTTGCCTGACGAATTTCCTGCTGACGTCGATTTCGAGTGGTACGAACA
GGAGTGCGAGAAGATTCTGCAGGCGATCGGATATACCTCTAAGGCCTGATAAATGGGCTT
GTCGGATTTTGCCGGCAAGCCCATTTTTGTGCTTGCATCTGCTCAAGAAGTGTCCTACAG
TTGCATCACGTACTAGGAAAGGAGGCGGATATGAACTTGATTACCTTCACAGCTCTATGC
GTCTTCGACGCCTGGATCTGGTTGCATAACCTTCACATCATTCTTGGAGCTTGAAATGGA
CGAAACCCTCGTCGAAGAGATCGTGCTGCGCTACCGCGCAATCGTGCCTATGACCACACT
GGGCGATCACCTGCTGTTCATGAAAGTGCTTCACGCGCACATGACGCACCCGCTTCAGCT
GGATGCACTGCTGATCGCGACCGACGAAGACATCGTTCATGACGTTGAAGGTATTGCGCA
ACACTTCGACACGAATGCGGGCGTTTATCGCGAGCTGTTTTGGCCGCGCTACGCTGCGCA
GTGCGGTGGCCAATGCAACCATGCGGGCCACTAGCCGATCGCGGCCTGTACCCCCGAAAG
CGCGGCCCACAGTTTAGTTCAACACGGAGAAAACAATGCAGAAAGCCAACACCCCGTTTC
GCACGCTCGCTATCCTCGTCGTGATCGTGCTTCTGGTAGGAATCGTCGGAAAAATGGATT
ACGAAGACGAGCTCCTCGAACTTGACATGTACTGCCAGAACGTGCATACTGGCGTCTGGC
CCGACTACGAGGGCAGCTATAAGGAGCAATGCCATGAAGGTCATCGCAAGACTGATCGTT
AGCGCGACGCTCGCGCTTGCTGGTGCTGCGCACGCCTGCGACAGGCAATGCACAGCAGCC
TGGTTCAACAGCGACCCGAAGACCCGCATCAGCGAGACCGGGCCAATCCTGTCCGCCTAT
GGCGAGGAAGGGAATGCAGGCATGGAAGCGGTGTCGCGCTGCAAGATGGACGTCGCTGTG
GTCATGCAGGGCAAGCCCGCCTATCAGCAGTTTATCAAGCAATGCTATCGTCTGTATCCT
GCGCGAGTACGGGATCAGATGTATCATGTCGATTATCGGTACGTTCCAACGGATCGAGTC
GATGTGTATATCCATATCGACAATAAAAAGCCCCGCTGAGCGGGGCTTCGTTTAGATGGC
AATGCCTGCGGACCATCCTCCGGACTTGTAGGCTGTGAGCTTCTGCTCGTCCTCAATATA
GGCTATCCAGCCCACTCGTGGCGTGCCGAACACCCAGGCCGACCCTGTCCAGACAGCGAC
TTTGCCGGCTTGCCCTGCCCATGCACCAGTGGGCGAAGCTGCCACGATGTATGTGTCGCC
CGATGCGGGGCTTCCAGGCGGTGTTGCGAGATCCCGATCCTTCACTGACAGCTGAGTGCC
GAAGCGCCCCAGCCACAGAAGGTTCGCGTCCATACCAGTGTTCCAGCCGTTCTCACCGAG
AGTCCAGCCGTAGCTCATTCCGCTTCGCGGTTCAGTAGATGCTGCCATTATGCGCCTCCG
TAAGAGTAACCATATCCGGCACCGTAACCGGACCGAGTGAATTCCCAATCGTGCATCTGA
AGGCTTTGAATACCATCACGCACGGCCCACAGCTGAATTCTCAGCCTTCCATAATTGGAC
CCCATCTCTGCAAGAGTGAAGGTGTCCGTAACCACACCAGTCAGCCCCGCATGCGTCACG
AGCACTGACCCCGCCTGGGTCAATAGGCGGCAAGTGTAAGTCGTGTTTGCTTCCGGGCCG
ATGCTTGCTGCCTCTGTATCAACGAGCGTCGCGGTCTGCTGCAAGCGGTCGCGATGCACC
CATGTCACAGTGATGTTCTGGTCTCCCCGAACCGTGTCCGGATAGGCCAGACTATTGATG
CGCAGCTTCTGCGGAGGGTAGGGGCGAGGCGCTCGAGCCTGGATCGTCACCGTTTGCGCT
GTAGCGGATGCTTCTGCAAGAGTACCCTTGGCCGTAGTGGGCAGAAGTTTGATCCGAGCT
ACTTCGCCTTGAGCGTACTCCACCTGATCTGTCTCAAAATACACATCAGGGAAGAACACG
CGAGCGCCTAGCGCATGTACCGCGGGCACAGAATCAAGCACCCCGCGTCCCACAGTCATA
GACGAGCTACTGATCGCGGTAACGATGACCAGTTCATTGTCGATGACTGCATAGGTGTTA
ATCTTGACAATGTCCAAGTCCATCCCGGACTGAATCGGGATCACCGTCTGGTTCGGGGTG
ATAGCAGCACTGAGGACAGCAGTAGGGCAGAAATCGACCGTGCCGGCCTCCTCGTACGCA
GTGTTCGTCGGGTTCGTATAGAGCTTGGCGTTACCCGCGTCACTCGTGGGCCGCACACCT
GTGGCCACCACGAAACCCGCTGTCGTCGGAATGCTACGCGCGTCAGTTTCACCCATGCGC
TGACACAGCTCCCAGAACGGCGCCTCGATGACGCAATGATACGGGCAAGGCGCGGGATCA
TTGTTGGGATCAGTCCAGCCGCTAGGCGGAGGAGGCGCATAGATGGCGCTCGACAGGGCG
AACACGTCTTCTACTGCGCTGATCTTGACGACGTTGCTATCGAGTGCGCCCAGCTCCACG
TTCGCCACCCGCATCACAAGCTGGCTGATCCCGTAGCGGGGCCAGCTAAGCACAAAGACG
TCACCAATGTTCAGCGATGCGGCCTTGCGGTTGGCGTAGATCGTGCCGCTGGCAAGTGGA
ACGGACAGCGCCTTGAGTGACCGTGCCGCTACCTTCGTGGCGATAGTTCCGTTCGTGAAT
CCTGGAAACTGCTTCGTCGTACTCACGACAGACTGCTGCTGTGCAGCCAGTGCGATGTCT
TGCACTGTGATCGAATTGTTCTTTCCTGTGCTTGCGTCCCAATAGACGACAGTCACCGAA
TTGACGAGCTCTCCTATGGTGTTACGTTTGAAATCCGAAATCTTATCGACAGAGGACTCG
TCCAGCACCAGCAACGAATTGACATCGTATCCACCGCGCGCGAGCTTGAGCACAAACTTT
CCTGACGTGCGATCCACGAACAAGGAACCGTCGATGTGCTTCAGTACCTCCTGAATGAAT
TCCTCAAGAGTTACTGAACGATCCCACAGGAGCGACATGCCCATGCCTTCGCTATACATC
TGATCGGCAGCAGCCTGAAACGTAGTGTCATCCACATCAGCTTCAGGATAGCCCATGCCC
CAATCGGGGTCAGTGAGGCACTCGCGGATGATGTGCGCTGGGTTCATATCCTGGCCGATC
GCTGCCTTCGCGATGTACCATTGAGCGATTCCGTTCTGGCGAACCAGTATGCGCTGGCCC
CAGAACGATGGGCGTTTCAGATACGGGTTCATGCCAAGATACACTTGACGGAACACAGCG
CTGACCACACCTCTGAAGCCAGGCACCATCGACCCCAGCTTGCTCACGAGGTAGCTGTTC
GGCGTCTGCGTGTCGCTACCCATGTCGATATCGACCGTGCCCGATACACCGCCTTCGCGT
TCCTCACCGCCGAACAATGTGTCAGCACTGATAGTGATTGCGCCGCCTGTAGCGTTACCA
GTCCAGGCAGTCTTTTCGTCGAACTCTACCTTGATGATCTTGTCGATCGGTCCATGACAG
AATACCATGTGCATGCCGAGATAATACTTATATCCGACTGTCTGCGCTTTGCTACTTCCG
CCCACGAGCCACCTCCACTGCTGCAAGCGCCATTCCGTCATTGCTGGTGGCTTCCAGCAG
CCTATCGGAATCGATGCCGTTGTTCAAGAAATCGGTATAGTCCAGGCCATACCGAGCGAA
CAGCTCCCGCACGCCCCGCGCACAGTAGCGCAGCTCGCGACAGTGTTCCATTCGGACGAT
CACACTGTTCGTCACTTCTTGCCTCCCTTCTTCTTCACAGGGACAGTTCGAAGATCTCCG
TACCACACCACATTCGGCCCGTCCATCTTACGGGTGCCAAACAGCACAGGGATTTCGCGA
CCGACTTCTGCTGTGGGCGCCTGAATCTCGTCCAGTCCGGCAGGAGGACGAGTCTCGGGC
TTGGGCATCATCGCATAGCTGACAACCAGCGCGACGATGAATACGATTGCATAGACCCAT
GCCATATCGTTCTCCTAAGCGATGGACGAACCGCTATACGGGTTCTTGGCTGGGATCCAA
GGGAACCCGCCGAAGTTGTCCAGATTGTTGAATTTGTTCTTGCACGTTTCTTTCAAGTGA
TCGCAGCCTGGGTAGATGGCCACAGTCTGTCCGCCCGCAAGGCTGGCCAGCGGTCTTGCC
ATCGTTACGGTGTCACCCGCATGGGCAGTAATGAATCGCGCGCTGCCGTCTGGGGCGACG
ATCATGCCGCCGGTGTAGTAACCGTCGGGCTGCAATGCTGCGCCCGCTACAGCGACGACC
AGCCCGCTCGCAATCGACAGCACAGCGCCCTCATGCTTGTAGAGCTCGCGATTGACGTTG
CAGCCCTTCAGGTACAGCGTGCGCCTGCACCCGTATTCGAAGCGCGCACGCAGGCCCGGG
CGCTTGATCGATGTGAAGACGGACTCGCACTGGACCTCGATCTGGTTACCGCTGGTCTTG
GCACCAACGACACGCCCTTTCCAATACACAATGAACTGAGCGTCCGGATCGCCATAGTGG
CCGCGATAGATCGTTACTGTGGTCACGTCTTCGGGCGCGAACGCGAGGTACTGTGAAGCG
AACGCATCGTCCCGAGGGAATACCAGCTTGATGGAATCCTTGAACGTGTCGGTCGTCTGT
TTCACGCTATCGCGCTTGACAGGTGACGGAGTGAAGGTCTGCCCCAGCCGGATGATCTCA
TTCACTCCGCTGACGAAGTTCCAGCGGGAAATCCCCTGCACGAATTCATAGAGCTCGATC
GGGGTTCCTGTTTCTAACGACGTCTCATAGTTTGCATATGTCATGACTCAGGTGTCTCCG
CTAGTGCGATGGAGGTCGATACCTGACCGACATCTCTGTGATTAAACGTCACCAGATCCG
TATCGAATCGGACGTGGGACATGAAGCACACAAAGTCGATATCGGTCGTTGCAAAGGCCG
TTCCGATCTGTGCGCTCAGCGACAGCGCTTCGTTCCCGTCGTTATCGGTGCTCCCGGACG
TTACTCGCGCAAAGATGCGAGTTCCGTTGAGGAGCTGGATCATGATATCCTTGACGGTGT
AGTATAGCGGATAACCGATCGGACTTACAGTGATTGAGCTTGCGGTGCTCGCCACGTCCT
GCAATATGACCAGATCACGGTTCCAGGAGGGCAACCAGAACGCGCGCTGCTTTCCGCGTC
GTGCATGGATCCATTTGCGCAGCTTCCACACGTCAGCTTTATTGTTCTTGATGAACGAGA
TCGTCTGCATGAAGCGCACCCAATTCGACGCAATGTCAATCTGGATGGGCCCTGAGCCGT
TGTCGAACATGTCGATCGTTCTGACAATCTTTTCGCTAAGGTCGCCCACCACAGCAGACC
GATCCGTCATAACGGGCTTGCCCCGATATGTGGGGAATGAACCATTATCGCCCAAGTCCT
TGTTCTGCGTCACAAGGAACTGCGCCTTAGCGGTGATATACTTGTTCGCCGCGCGGCTGT
ATTCGACCCCGCTGAGCGTCCTGGCAAAGCGAAGGGGCGCGATATAACACTTGGGCCAAT
TCTTGTCCAGGGGCAGCTTAAGATTGACGCCAGTAGGGGTCACCGTCGTGATCTCCAGCG
CTGCCAGATTGGTATCGGATGCCCACAGCATGATCAGGTCGTCGTCGCGGTAGTCAGCGT
TCGCAGTATCGAACGCAATGAACGACGAACCAGCGGTAAGACCGTTCAGTAGAGGAGTCA
CTTCATCCCAGACTGCAATGCCATAGACGCGGTGTGCCCATTGCGTGCTGATCGCTTTCG
CTCGGCTGAACTGCTGCTCGTCCAGAAGGAACTTGTGTGAGAAAGACTGGCGGGGAGCTT
CGCGCAGCGCAAGACGCTGTTCGTTGTTGAAGCTATCGAGCAGATCGGTCTTCCATTCCA
TCGTCTCGTCGTACCCTGTATCGGGCACAAAAGGCCACAGCACCACACGGCGACCAGTCA
CCGTGAGCGTAGGCGTTTCGGACGGGAAATGGAAGCTATACACAGCATCGACGATCGGAG
GGCCGTTCGTGCTAATGTTGATGGTATAGATGCGAGCTTCGACAGCCGCAAAGTAGGTGG
GCGCCGCTTCGGGTTGCGTGAGAGTAATGCCTTCAGTTCCGACCTGTGTCAAAGAGGACA
ACAGCTTAGGCGTAAAGTAGGCTGACCACACTTCCACGGTTCGAACCTGTGCAGACAATA
GGTTGCCAAGTGCGATCACGCCCGGATTCACGTGGATGCGATTGTAATAGTCGTCCAGGA
AAGACTTCTGGCGATATCCCGTTAGTGTGTCCTGGGGCTCAAGAGAGATTGGCGTATTGT
CTGTGATCGTCCCGTATGAGAAACCAGGCGCAAGCATGACAGGACGATTCGGGGGAACCG
AACCATCTACGAAGTCAAGCTCGTCATAACCCGCCGCAATCTCAGGCGGGTTCTTCTGGC
GCCATTTGTTGTAATCCTGGACCCCGTAGGTCGTGATCACTGCCATGTTACACCACCTTC
AGATAGGCGATTCCGCGCTGGAAGCTGCGCCCTCCCTTGTTGTACCACGGGAAGACCTTC
CAAGTGTCAGAGCCTAGAGTGAATTCGTCGCCGGGCAGATAGTTTGTCATATCCATGTAG
CGGATGCCCGGGACGACACCGATAGGGTGCAGATAGAGGTTCGCTCGATTGACAGATACG
ACATTCGGAGTCAGCAGACCGATATTGTTAAGAGGATTAGGGGCCAGGTCTCGAATCATG
CGGTCATGAGTCCCTCCCCCTTGGCAGGCCTGAGCAAGGTGTGTCGTGGTGGATTGTGTG
CCGGACCCTGCCCAGTTGTCGAATGCGTCAAAAGCACAGCGCACAGCTGAGCCAGATCTC
GCGGAAGAAAGAGGGACTTCAGCGGCCCGGAACGGGACTTCCTCCTGGGACCAAGGGGAG
CCAATTTCGCTCCCAAGCCACGTCGACGACGTGGTGGCATTCGTCACGTGCTCCGCGCCG
GTCGCGTAGAAGAAACGACCACCACCGGGCGTCGAGGCGTTATAAAGATCGAGCGAACCG
AACCCGAGGCGCTGAAACACACCTGTTGAAATCTCAAGCTCCAGATAAATCGTCTTGCTG
TCCGGAGCGAACAGGTAATAAGATGGAAACGGCCCGAAGTTCGTCACCATGGGCAGAAAA
GCGTGATATTGTTCGCTGCCCGCTGTCCCCGCTGGACGCACAGGGTATCCAGGCTGACGG
TCCCAGGCGCTGCCTCCCGCGTATCCATCGGAACCATTCAGAGTGATGCCATATTTGCTG
GCCTGCGTAGAGCCATTGACCAGCAATGAACCGTTCTGGCAGGAACGAAGATTGAAATAG
GCTGTACCTTTCTGCAGACAAAGCTCTTGGCCCGACGCCCACGCTACCCAGCGATTGACT
GTCCAGCCTTGTGCAATAGCGAACAGGCGCAGCTTATCCAGCAGATCTGTCGCTCCGGTT
GCGGTTCCAGTTTCGTATGCCATTCAATTCTCCTTAGGCGGGAAGGGCCAACGCCCAGTA
TTCTTGAACTTCGGTACGCGCGACGTTCTGAAAGACAATGTGGTTAGTCCCGCCTACTGT
TGTCGTGTTCTCGGAAGAGTTACTGAAGCCCGAGATAGCGAATACACCCTCGAGCTCGCC
CCATACTGCCAACGTCGGAAGTCGTTGCAATAGGATAAGCGGTTGAGTGATATAACCCCC
TCCTAGAACCTCGCGATATGGAAGTGTTCCGTCTCCGACAGTAGTGCTATGCACACAAGT
AGGCCAGACACCGATCGCAGGTCCGCCCGTAGCAAAAGGCGACGTAGGGCTCATCGTCAT
CCGCGTGATTGTATTCGGATTCGAAGACGTTGCTCGTTGCCCTGTTGAACGCCATGTCCC
GTCAGGAAGCCTTATGTAAAGAGTCGAATTGGCGAATTGGACAGTAGGTGAAGAGCATGC
GGGCGTCGGGAACACGCTGTGAGTGAAATCGTTATACGAATAACGCCATTCCACGCCCCG
AGCGCTGTTGCTCGGAATCATTGACCCGCCTACCGCTATCGGATAAGGATATTGGCCTGG
CGTTGCGTACGGCAAGAAGAACCCAAGATAGCCCCCCTCGAAACTTGTGGAGACTTTTAA
CCCGAACCTGAAAGAACGACCTGTCGCTGCAAACCAATAAGGCATTGATGTGTTCCAACA
AGGAACCATCGGGCAGATCATGGGATTGACGTCACCGTAACTAGACAACGCGCCAGGCTG
CATAAAGAAATCTAGTACGTTGGCGTCATATCCGGTATAACCCATCAAGAAGAGGTTATA
CCAGCCGTTCGAAGCGTCGTATTCGCAACGAATACCCGTGTAGATATTCTCAGTTCCGGA
ATTGCCGGGCGCTTTGAGAAGTGCCTCACTACCGAAATGATTCGCAATGGTCCCATCATT
CAACTGAAGTAACAAAGAACGCCACGACACCGTGGCGCCGGAACCACTTTGCACGCGATC
AATCAGGATACGCCAATACACATGAGCGCCCGGTGTTCCGGGCACAGCGAAATCCTTCAT
CTCGTTCTGAGCGTATGTTGGATTCGAACTTACTGTCAGTGCGGTAGTCCAAGTCGAATT
ATCGTCAGAGTATTGGAGACGGAAGTTCTGTAGCATCGTCGAGACGTCGCTACTGTTCGG
CGCCCACATACGAACATTCTTGATTTCTCGTGCTGCACGCAATGTCAATGCGATGTAGCT
GGTGCCAGAAGCGTAGCCAGTGCATTGCGTATAACCCTCAGCGGTGTTCGGGTTATTGGT
GTTGAGGGAGCGTGGCGTATAGCGAAACGAGTGGAGGATCTTCCGGGCCGTTGCGGAAGT
AGGCTCCGTCATGTTTGTCGTCATGCCTGCTACGCCATCTCGGAATTGGCGATTCACAAT
CCAATCTTGATTCGCCGCAACGAGCGCAGCATTCGTCGTGAGGAAGGAAATGATCTTACC
GAACAGATCCTCCAAGTTCGACGCGGTGCCTACTTCGTTTGCCATTTAATTCTCCTCTTA
GTAGCCCAGCGCGCGTTGATTGCGCTGAACGACGTTCATAATCAACTGTTCGCCATCGTC
AGTTCCGAGGTAATCTCCGACAATGGACGGATCCAGCACGTTGATGATCTTCGGTGTGAC
CACAGTAGGCTGAGCCGCTGCCTGGTCGCCCTGACCCTGTGCTGCTGTACCCTTGCGAAC
TTGTGTGGGCGTACTGACCGTCACCTGCTCACCGGGCGTTGCCCTAAATGCTACCATTTG
TGAGTCTGCTCCACCAGTACCCCCGACGGTGAAGTTACCGCCGGTCATAAAGCCCTGAGT
CTGCTGGGAGCGGATCGCTGCCACCTGCGCCATACCCTGCGCCACCACGACAGCAGCCAT
CGCCGCTCCGTAGATACCGCCCTGCGCCAGCGCCTTAGTTGCGCCCTCGTAGGTGTTCAT
GATCGCCTGGGTGATTGCCGCAGCCTTACCGATAGCAGCCAGCTCCTTGATGTTGGAGCT
TTGCAGGCTGGCAAGCCCGGAGAAGAAGTTCCGCATGTTCTGCGTCTTCAACTCGTTCTC
ACGCTGCGCCAGCTGAACCTTGGCGTTCGAGTAGTCCTGCTCGCTGATCAGATCGTTCTG
CCTCAGCATGTCGAGCTGGGCATAGTATGTCTGGATCAACTCCAGCTGAGCCTGCATCTG
GCCTTGCATCCCAGCGGTGTCGATACCCATGCCTCCCAGCATGCCCATCACCTGGCCTTG
TGCGTCGCCCGCAGTGAAGCCACTGCTAGGGTTGGCCAGCAGGTTGTTCATCGCTTCGAT
CTGTGTGTTGAACGCGCGGCGCTGCTCGACGCTGTTCGCAAGCATTTGATCCTGAGCTGC
CACCAGTTCGTTCTGGCGTTGCAGCATCTCGATCTTGCTGCGAAGCGCAGCGGTTTCCTC
TTGCGTGAGCGTGATGCCCTGCGAGCGCAGATCCTTCTCGATCTTCATGTACTCGGACTC
AACCTGCCGCGCACGCGAAGACATACCGAGCAGACGGATCTGTTCGTCCATCTCGCGATT
CACCTTGCCCAGCGGATCGATGATGTCCTGATAGTGGAACTTGAGGAGCTCGAGGTAACG
TGCCTGATCCTGTGTGGTTATCAAACCCTTGGCGACCGCCTTGTTGAGCGTCTCCTCAGC
CTTTGCCATTTCCAGCTTCGCACCTTCGACCGGAGCGATCGTGTTCAACAGGCTGCGCAG
TTCGTTCTTGAGCTTCTCAAGTTCTTTGCGCTGCTTCTCCAGTTCCTTCTCGTCCGGGCC
GGGGGCCTTGAAGGTGCCCATCGCTTGGTTCAGATCCACGCCGGTCTGGTTGCCCAAAGC
TGCGGCGCGTTGCTTGCCGATTGCCTGCGCACGGGTGAAGACGCCATCGACCCACTTCTC
CATGAACCCGCCTTGCTGCTGGAAGCCGTCATCGATAGACTGCGCGATGTTCTGACCGTA
GGTCTCGAAGAACTTCGTATCGACTTGCTTCCGGTCAAACTTGACGGTCTCAAGCAGATC
CATCCCGACTGCGCTGCGCATCTTATTCACACCGTTGATCACGGTATTCACAAGGTTCTC
GACAACACCGACGACCGCGTTATAGGTGCGGTTGAACATCTCCTTGATCGCGTCAGGCAC
ACCAGCGAAAGCCCTGACCACAGCGATGCCTAGTCCGGTCAGCAGCCCAGCGATCGCGTC
AATGGTGCGCGCGATACCCTTCGCCACCCCAGCAAAGCCCGAACCGACACCATCATAGAA
GCTGGTATATTGGGCAAGCCAGTTTTTCGTTTCCTGGTCGGTTGCTTGTGTGATCGACTG
ATACACGTCCGCAACGATGTCGCCCAGCCCCGAGAACGCGCTGGAAGCGAGCTCCTTCAG
ATCACTGAATACGACCGCGATGTCCTCGCCCACAGCACGGAACACATCGCCCAGCGTAGT
GACGTCGTCCAGGCCTACGTTGATCTCGTCCCGCATGAGCGTGAGGTAAGTCACGACAGC
AGTGAGCGCGATCACAACAGCGGCCAGCGGATTCGCGGCAAGCACGGCCCACAGCGCCCG
CACAGCGTTCGTTACGGCAGTGATCGCCGTAGGTACGAAGGCAACAGCGATCGCGGCCCC
TACCGACAGCGCTGCCGCTGCAACCATGTCCATATTATCAGCGATCCACTTCATCGCTTG
GCCCAGCGTGTTACTGATGCCCAGTGCAGAATCGGTCTGGCCAATCCAGCGCATGAAGGC
GTTACTGATCAGCGTGAAGCCCTGCGAGATGGTGAAGGATGCCTTGCCGAACTTGGCGTC
AATCTCTTCCGACCCAGCAAGCATGGCATCGAAGAACGCCTTGGAGGTGAGCTTGCCGTC
CAGCATCATCTTCCGGAGCTTGCCGATGCTACCCTCGGCGCCGTCAATGTGGTTCGCGAC
GACTTGCAGGATCGCAGGCGCACCTTCCAAAATGGAGTTGAATTCTTCCGCTCGAACGAT
GCCGCTTCCCAGCGCCTGGCCCATCTGCATGAGCGCGCCTTGCGCCTGCTGTGCGCTGGT
GCTGCTGGCCGCAAGCGTCTTGCCCACGTTCTCCGTGAATTTGATCAGCTGGGCCTGGGA
TGCGCCAAGCTCTTTACCCGATCGCGCGGTTCGACTGTAGAGCTCGACGATATCCGTGAA
GCCCTGGCGGGTGTTCTGTGCTGTCTTGTAGAGCTGCTCCTGCACGGCAGCAGCTTCCGC
GATGTCCTTGGTCGCGATGCGAATCTGCCCGGACGCGCTTGCCCATGCGTCGGCATACTT
GCGGATCTGATCGACTGCGATAAAAGCAGTCAGCGCGCCGAGGGCGCGCTTGAGGATATC
AACACCGCTTGCAGCTTTCTCAGCGGTGTTGCCTACGTCCTCCAGGTTCCGCTTAACGAC
CCGAGAACCGTCTTCTCGGATTCGAATGTCAATAGTTTCGGTCGCCATTACAGCTCCTCT
TTCAAAGCGTTCTGCAACAGGCTTCCGGCGCCCTGCGCAGCAGCGACCCCGACTAAGACT
GCCTTCTCAACAAAGCCCGACGGGGCTTGTGCTGACCAGCCGTCATTCAGCCGTCCGATA
TATGGAAGGTTGTTCGTGATATGGATTGCGGAACCGCCCTTGTACTTAGCGATCTCCGTC
TGACCTTCTGCGATTGCAGCTTGACCAGTTTGATCGAGTGCGTCGCGCGTCCCACTGGCA
GGCCCACCGAGATTGACCTGCCAGTTAGAACGCGCGCGACCCGTATCGACAGGGGTGGCG
ATGACCACAGCGCTATCAACAGCGAGCGCGACCTTCCGCGTCATGGCATCAGCGTTCTTT
TCGATACGCTTACCCATCACACGAATCCGTCCCGCGAACTGTCTCAAGTCGGCCATCTAT
TTGCCCGCTCCTGTTTGTTGCGTGGCGCTCTTCAACTTCTTCGCTTTGAAGTCGAGATAG
ACCTTGTCGAGTGCCTGTATGTGGTAGAACAGATCCTCGCGTTGCTCTCCCTCGATCTCG
TTTGCGTCCGCCCACTCCTTTATGGCGAGCCAGCTGATTGGACCTTCTGTGTTATAGCCA
GTTCCGCGACATGTGGTCAAGTCAAGAAAGGCCATATGGTACATCTCCAGTCCTAGCAGC
AGTTCAGGGGCATTGGCAATCTTCTCCGGTAGCGGTAAGCGTTGTTGAACGCACTGTTGA
ATGATGAACTTTTCTGTGGGGCCCTGCTCCAGGTAGTAGAGCAGGACGTCCGTTAGTTTT
TTGCGTCAGCCTCCCGCACCTCCGCGCGGAAGAGTGCAGCACGCTGGCTCTGCTCCTGGA
TGTCACGGAACAGGTCCGGCAGGTCGTCGAACAGCTTGATGCAGTTCTCGACCGAGAATT
CCATCGCGTTGCCGTTCTCGTCCTCGACGTTCTCCCAGCCAATGACGACGGTCTCGGCAT
ACACTTCTTTGACGATGCGCTCCACCAGCGCGGATTCCATCGTCTCGTTCTGGATCTGGC
GACGGTAGGGCTTCACGCGCGCTTCCATCCGTTTGGCGTAGGCGTTGTTCGAACCGCCCG
CACGCGCCACGCGAATGCAGATCGGCTTGCCCTTGCTGTTTTCACCGTACTCGAGCAGGA
TGCCGTCCTTCTCGAGGGTGTTGTCGGTCTTGAATTGTTTGTAGAGACTCATTAGATCAC
CTCATGGTGCAGTTTCAGGGCGCGGGCACCATTGCTCGCGCCCCTGTAGCTTACACGCCG
CCAGCCACGTCAGGCAAGTAGGAGAAGCTCTGGAAGAGCAGGGTGTGGCCGAATTTGGAT
TCAGCCGCGTTGTTTTCCAGAGGCAGAGTGATCGCCTGGTCTTGCTCAACCGCCAGACGA
CCATCACCCAGCGACAGCAGCGGGATATCCCACAGCAGCGCGAGGTTCTTCTTCAGCATG
ACGATATCGACCGTCACGTCGCTGTTATTGCGCACAGCCTGCACAGCGGTGACATCCGCG
AAGTAGGCCGTCGTACTGCCGCCCACCTCGAAGGTGCCCGCGCTGGTGTCGAATGCGCCC
AGCACGCCGATCGCCTTGTTCGGGCTCACGTTGTTATTCACCGACAGCGACATTTCGGTC
GCAAAGGCAAACAACGGAGTCGGAGCGGCGTCAGTGCTGCTGGCCAGCGCCAACTTGATA
CGGCTGAAGTCGCTGGAGGTGTTGAACGCGGAACCCGGAACCAGCGTGGGGCGGGTGCCT
GACTTCACGCCCTGGGTGCCAGTGCGCTGTTCGTTATCCACCGCAACGAAGGTGAGATCC
ATCGTCACCTTGTCAGCCTGTGCGATGTTGAGCGTCAGCTCGTTCGGCACAGCGCCCACA
AGGTATTCGGACATCGTACCGTTGGCGTCGGCGCCCAGCGTGCGCTCCACTTGGTAGGTC
CGGCGCTTGATCAGCGTCGGATCGCTTTCGTTCTTCAGCACAGAACCGAAGAAGATGCGA
ATCGTCTTGCCGGTGCCAACTTCCGCTTGCGGGGTCCAGGACACCTTGTCGAATTCCAGG
TATGCAGCGTTGATGACGCTCACACGAGCGAATCCACGGTTGTTCGTGAATTGCGTGCCA
ACAGCATCGCCGCCCACATACACCCATTCGCCAGCAATCAGCCCCAGCGTGGTCAGGTCG
AATACACCGCTGGAACGGGTCAGACGAACAAGCGAACCGTTCATCGTAACGTCCAGAGTC
GCGCTTGCGAACTGATAGCCCACAGCTTCCAACTTGGCAGCAGCAGGCGGGGACGCTTCG
TCGGAGATCACCTCGTTGATGACCACAGCGGTCGCAGTGCTCGCGCTGGCGACGCTCTTC
AGGCCGTTGTTGGCGGTGACACCGAAGCCCGACGCCATGATCAGCTGGTTGGCCAGGAAG
CCGGTCAGCCCTGCTGCGGCAGCGTAGGTCTTGGACGCGCCGGTGACACCAGTGATCGGC
GTTGCAGCCGTGTTCATGGGTGCAGTGGTTTTCTTCTCACGGATGTCCGCGAAGAAGAAG
CCTTGCAGCAGGCGGGTCGTGTTGTTGAAGGTCAGATCCTGGTTGAAACCGCCGGAGGCG
TCCAGGTCAGTCGTCACGCCCTTCTTGCGCTGGCGCGACGGGTTGATGGGGTTGCGGGCC
ACGGTAGCAATCTGGCCGCCGAAGTCCGCATAGGAGTTCGGTTCCAGAGGATACCAGACC
GGCGAACCGGGAAGGGTTTTGAGAGAAGCCTCTTCGGCGAACCGGAGGCCGGTTACGTTC
GAATCGATCTTGTTTGCCATGTGATCACCTCACGTCGTCATAAGAGAAGGTTGCCAGGAC
GTTCAATTGCTCAAACGCCCCACTCGTCCCAGCTTCATTCATCCGCACGTCTCGGAACCA
GACATTTGGATGTCGGGCGGCCTGGAAAGCATCCACCACGAGCTGGGCTGCATCGTATCC
GGCCTTGGAGCCATCTCCCACGGGGGCGAACACTTGGATATAGACTGTTCCAGTGCGCGA
CCAGCGCTTCGCACCTTGATCGTCGGATAACGATGCTTGACGACCAGTTGCGTGCCGGAT
TGTAGCACGAGCCCACACTGTCTCGCTAGTGGGAGCACTGCCGGGCACATCAGTCCAAAC
GGCAGGGAACCCCGTCGGATCCCATGCCGCCTTGAAAAACCCGAGGATGATATCGCGCGC
TTCATCGAATGTCATCGCTTCACCCCGAATACATACAGGACGACCAGCGGGCCAGGCTTG
AGTGCTTGCGCCCAATCGATATTCCAGCGAACACCGTCGTCAAGAATTGCGTGGAAGTCT
TCCAGCGACACGTCTGTCGGTGCAACGAGTGCGACCTGCTCAACACGATCCAGGAGCTCT
TCCTTGACAATATCGCGCCCCAGGCCCGAACCAGACGCAGGCACGAACACAGCAGGCAGC
ACCTTGGCCAGCGCTACAGCAGGGGCGCCCGGGCCCTTCCAGGGCTTGCTAGTGTCAGCG
GCGGTAGCATCCAGCTTCTGGAGTGTGACCTGCCTACCGTTCTCGGCGATCAGTTCCTGT
GCCAGCTGGACGAATTCATTGTAGTCGGTAGTCATAGATCACCTGATAACCTGGCTTGCC
GAATAAACCAGCCCCCGCAACAGCATATCCGCCGCAGGGTACGGACGGAACAGCATAGGG
GTGGCGCCAAGGCCGGTCTGCGGAACAGCGAATTCCGTCTCGATCGGGCCGACCTTCTTT
TTCGTTGCGACAACAGTGAGCCCGCTGGCATCAACCTTGAGATCAGGCGCCAGTTCAGCA
GTCAGAGCGCGCAAAGCGTATTCGCAGCACGCGCGGCGCAGCTTCACAGGGATATCCGTG
TCAGCGAAGTCGGCTGCACCAGTGCGAGGCCATGCCAGAGCCTGTGTGGTCGTCTTCTTG
CTACCGATGAAGCGCTGTCCGAAGCGCCCCTCGATGTAATCTGTGGCCTTGATCAGCGCG
GACTGCTTCACAGCATCCGCACCCGTCCAGCCTGTCATTCCGCGGTCAGCAAAGTACCCA
TCAGCTTCCGCGATAGTGACGTACGAAGTGGCGTCTGCCTTTGCAGTACCGTCTTCAACA
ACGAACGCCATTATGCACCTCCGATCTTAGAGAAGCGACCCAGGCCGGTATCGAGCAGCA
CTTCGAAGCCAGCTGCCGGAGTGACCACAGCAGCGCGCTCGACATAGTCCAGCGTCATGG
ATTCGCCTGCCGCGAGGGCCGGAGTCTTGATCGTTCGGTTGCTGGTTCCCGCGATGGTTG
CAGCGGTAGTTCCGGCAGTTGCGCCGGCTACGCTCTTGAGTGCGAGGCGCGCACCAGCAG
GCAGGGTGACCGCTCCGACGGCAGGAGTATCGATCAGCGCCGCGAATGCTGTGCGGTTCT
GACGCCCTTTCCGGTTCTTTGCGATCACCTGTGCCTGCTTCATTGCTTAGCCCCCGCGAG
TGTTGCGACGTGCGCGAGCAGCGTCGATCGGCGCCTTCAGGTTGCTTGCCAGCTCCTTCA
GGTTGATGCCGCTTTCGCGGATCATCTGCTGGCGGGCAGCACGCGCTTCCAGGTTCTTGC
GCTGGGCGGCCAGATAGTCCTGGATGTCGTTGCCGGTGCTGCGCTGCGGACGCGCTTCTT
CCAGCTTGACGCGAAGCTCGTCTTCCTTCGCACGCGCTTCTTCGAAGGCCTTATCAACCT
CGGCCTTGTGCGCGCGAATCTTGCTCACGAGCTCTTCTTGCTCGGCCAGCGTCTCTTCGA
GCGCCGCGATGTCATCGGCTGCCACGCCAGCGGAACCTTCCATCTTGGGCTGTTCGGCTT
GCCCAGCATCGAGGTCATCAGCTCGCTGGTCGGATTCGGTTGCCTGCGAGGCTTGCGGGG
CCGTTTGCGGGGCGGCAGGGGCCGCGCTTTGCTGCTGCTCACCGCTACCCCCTTGGCCTT
GTGCTTGCGGGGCCTGCTGTTCGGCGCTGGGCGCGGTGTAGCCGGTTGCAGTGGTACGGG
AAAAGCCAGGCGCCGCCGCAGCGACGGAATCGCGGGTCAGGGTCTGGTCGGATGCCAGCA
TCTTCACGGTATCCAGGCGCGGGAGACCGTCGGCTGTCCAATGGTTGTCGTTCTTGACAT
CCAGCTTGAGCAGCGCTTCAACAATCTTGTTCGTCATGAGTCATTCTCCGGTGTGTGGTT
AAGAAACGGACGCCCGCATTATACTGACCACATGCAGGCGTCCGCTACCCTTAGTCGTCG
CCGAGAACGCTATAGACGATATGGAGCTCACCGCTGGCAGTCAGGGACTGGCTGTCGGCG
CTGATGTTGGCATCGTCGATCAGCAGGTTCAGGTTGAGTTCCAGGGATCCGTCGGTGTTG
TCCAGGATCACTTGCGTGGCATTGGTGCCACGTGCGCGCGGGGACACGCCAGCGGTGGCT
GCACCCAGCGCGGTCGAAGCAATGATATCCACTTCGGAACCGCTCAGAGTGGCGTCAGCG
GTCGGCGCGCTGCCGATGCTGTAGTCACCGTCGAACGTATCCTGCACGCCAGCGGCGGTC
GCCTTGGTGATCTGCACGTACGACACCGCACCGAGGAACAGGATGTTCCCTTCCGGCAGG
TCGCCCAGGACAGCAGTGCCGAAGCCCACGCCGGTCGCGCCATTGACAGTGATCGGGAGC
TTGTTCAGCTTGACGACCTGCTTGACAACGTCTTGACGGGCAGGGGAGCCCCGGCTGGTA
GAACGAGGAAGGCCTTTCATGATCGCATCTCCTTTAGAGGGTTAAATCCGAAACAGGGGT
CTTGCGACCCCTGTTCCAGTGCAGATTAGAACTCGCGGGTGATGAGACGCGCGATCCGGA
TCTGCTTCCGCTCCGAGAAGACACGCTTCCACGAATCGGCGTGAGCCAGGTTGTTCGTGG
TGGAGGCGTTGCTCGGGCCACCGTTCGGCGGGGTGCCAGCGTAGGCGTGGCCGACCGGAT
GGATGATCCACTCGGTGCGGTTGAACAGGATGTCCTGGCCACCGCCGTTACCTGCGCTCG
GCTTGCGATCGACTTCCGTCGGCACCTTGGGCGAACCCATGCCACCGCGAACAGCACCAC
GACCGAACAGCCAGGTGTTGAACACACCGCTGGAGCGAGGCACGCCGTCATCGACGATCA
CTTCGCGGCCCAGGAAGGTCGGGATGCGAACCGCATTGCCATTGACGCTGTCCGACACGA
AGTCGATCAGGTTGTTCTTCAGCATGCGCGCATAGACAATGGAATGCACCATCACCATCG
TGAGGTCTTCCATGCTGTCGCCCATGGTAGCGGTGGCGTCGATGAACGACTCCGCGCTGA
AGTTCGTCACACCATCCACGAAGCTGGCGCCGGAAACGTCATGGGTCATGTCGTTCTGGA
CGTGCTCGGTGCCGGTGGGCGCTGCGGCGTTGTCCGCGAACACACCGTTCAGGGTCGCAA
CGAATGCGGCCTGCTGGCGGCGGGTCCAGTAGTCGGACACACGGTTGGCGATCGCTTGCA
TCGGATCCGCACCAGCGAGGTCGCCGGACAGGTCCATGGAGCTCCAGGAGTTGTTCCGCG
ACAGGCGGACCTGGATCTCGGTAGCAGTGCCGATCTTGTTGGGCGTGCTGTCGGTCGCCG
GATCATCGGTCGAGACGTTTTCGGCGTCGTTGTCCAGATCCTTGAAGGACGGCTCGTTGA
AGGTCAGACCGCCACCCGCGAGGGCGCTGTTGAGTTGGGCATCCAGGACGATCGCGCCGG
AACGGATCAGGCGGGACTTTTCCTGGGTCATTTGCTGGACGTAGGGCGAGAAGATCTCGG
GAACGACGACGTCAGCGATACGAGTTACACCGGAAGCCATATGAATTCTCCTAAAAAGGT
TCGGATCAAGTTTGCTAGACTCGAATCCAGCACCCCATGGCCTGGCGTTCGATCGTTAGT
ATGGCTCATGCCACAATGATCGCAATGTAGCATGAGCCAGTCTAGCTTGCAAGCCCCTGA
GTTTGATTATTTGCGGGGCTGCGGGCGCGGGCCCCCGATGGTAGTGCCAGCGCTCTTGGC
CATCTGCTCGGCACGGGAACGGTTCTCCTTGAGGATCCGACCCTGCTCCGTCATGTTCCA
GCCATCAGCGCTCCACGGGTTCGCACCGCCACCGCTGCCGCTACGGTTGCCACCAGCGCC
ACCTCCTTGCGACGGGCCCCACCAGTGAGTCTTCTTGGACTGCATCTCGGTCAGCCACAC
GGTGGCATCGATACCGGGAGTCACGCCCACGCCGTCACGGGTAACGACCTTGCCTTCTTC
ATTGACTTCGAGCATGCGCTCGGCGTACAGAAGGGCGTCTTCCACTGCACTGGACTGGAA
GCCCTGTGCCTTGCCCACAGCTTCGCGGACAGCGTCGTGGATGGTGCGGGTCTTTTCCTT
GGACTGGAACTGCTCGACGACGCCAGAGAGCTCCTGCACGCGCTGAGCCAGCTGGCCCTT
CTCGCGCTCGAGCGGGGCGACACGGGTCTTGATGCGGCCTTCGACCAGTTCGTTGAGCTT
GTTCTCATCCAGCTTGCCTGCGGCCGCAGCTTCCAGTTCCGGAACGCGGTCCAGGGTTGC
CAGCACGTCCTCGATCTTACGATCGCCGAGCAAGCCAAGACGCTCACGGACGGCCTTGTG
GTCATTCCGTTCCTTGGTGAGCGCACCCTGCAGACGTTCGACATCCGCCTGGGTCTTCAT
TCCTTCCACCTGGAGTTCGAACTTACCGTTCTTCTCCACATACTCTTTCTTGACGTCTTC
GGGAAGGTCGTCGATCGAATCAAGAATCGCTTTGAGAGCCATGCTCCATCTCCTAGTCAA
ATGCCACTCATGTGGCGGGTTGCTGACCACACCATTGTGATCAATACTTACTCGGGTCCA
GCCCAGCGGCACGGAATGCGTCTGCGTGCTTCTGTGCAAGCTCCCGAAGGGTTAATTCGT
CTCCGTTGCGATGAACGAACTTGTCCAGTTCCAATCCACCGTCGCGGAACAGCTTTGCCT
TCGTTACGCCCATCACTTCATCCTGGAACGCAACAGACTGACCCTTGAGCCACGTCTGGT
AGCTGGTCGAGGCAGGGATCGGCCCCACCAGTTCCCGCACCCTTCCGCGCGCCCACTTGT
CGTAATCACCTTTCGTTCCACGCGGCAGCGCGTCACGAGAGCTGATGTCGCCCAGGCCGT
TCTTGTCTGCATATTCCTTCACCAGGATCTTTTCCGTGGTAGGCTTTGCAGGACGGTCAC
CGGCGAGCGTACCATCAATCGCGGCGATACGCAAGGACCGGCAGTTGAAATGCAACGGCG
GGCGCGGACCCTTGCCCACGGGGAACGTCTTTCCATCAAGGCTGCGACAGATGGGCGTCG
TCCTGGAGTCCAGCGTCGCCACGAACTGTTCCGCGGTAAGGATGTCCGCATTGTCAGCGA
ACCATGCGTCGCGCGCACCGTTGGCAATGTGCTGCACAGCAGTCCGGGTGATCGTTTGGA
TCTGCCTACGGGTGATCTCCGTTACACCGTCAGCGCCTCTGAGCGTACCAGTCCCGACCA
CACGTCGCGCGATGGTCGCCATATCCTCGCCTGCTACCATGCCCGCCTGGATGGAGCTAT
GGATGCGCCGAATGTCGTCCGCTGCCATCGTGTCTGCCCATTCCTTGAGGATGCGGCCCT
CGAACGGGCGAGACAGTGCGATCTGGCGCAGGAACCGCGCGCTGGGCATGACGGTCTCGA
CCGCTACAGGCAGCACCGTCTTGAAGATGGCGTCAAGTTGAATCGGTTCCTGGTACGACA
GCTGAACCATTTCCTCGGTCAGGAACTTGGTTGCTTCGTCCCACGACTCTTTTCGGATTG
CGGCCAGCGTACCCTGCAAGGCTTCCAGACGCTGCCACTCCACAGGCTTCGTCAGACCTT
GCATCGTGCGCAGCTTGTCGCGAATACGACGAGCCAACTCATCCTCGCTGGCGTTCAGAA
TCGTGGTCATGCGGTTGCGGACATACCCGCTATAGCGCAGGATGTAGATCTGATGACGCA
CCAGTGCGTCCAGAAACTCCTCGTTGGCTGTCTTGGCCATGGGTTACGCTCCCGCGCCGC
CTTGCGGGGGCTGCTGTTGCTGCTGCCCACCTTGCTGGCCCTGCTGCCCCTGCATACCGG
GCTCCAGCGCTGGGTCGCCCCCTGCGCCCGTACCGCCGCCCGACGGTGCGTTGGCGTCCT
CTTCCCCGATCTCTTCCATCTCGGCATCGAAGTCCATCTTCGTTACACCGCGATCCACCA
GCATCGCATGGATGGACTTCTTGGACAGCGGTGCGCCCATGGTGCGCGCGGTCATGAAAT
CGACTAGATCCTTACCTGACATCTCGAAGTCAGCGAACTCGAGGTTCGGAGTGACTTTCA
CGTCGTCGGGGTTGGCGCCCATCCACTGAGCGCACATGCGCAACAGCGATTCCAGCGCAG
ACGCACCAGTCATCGCAATCTGGTTCAGCGTCGCAGTTTGTGCGCCAATACGGGTCTGCA
GAGCGGAGCCGCTTTCGGTGTTGTTACCGCCAGAGTTGATCAGCTGACCCGAACGGGTTT
CTGCACGCTTGCGGTCGTTCTCGAGCGCCTGGCGCTGCTCGGACAGGCCCTGCGAATTGA
CGCCGATGTACTTCGCGTCACCGCCTTGCTCCACTTCGATCATGCTGCCTGCACCAGTGC
GGAGCGGTGTGCCTTCGTCCTCAGCGGCATCGGTTTTCTTGACACCGCCCACGACGACCA
GCGTGTCCTGGCCCTGCATGAACAAGTTCTGGCGGTAGTCCGCTTCGCCCCGATACACTG
CAAGCGCAAGCCTGCCCAGGCCCAGGAGCGGAGGTTCGTCCGGCGTGCTGACGATGTCCT
TCGTATTGACGAACACGAACGGAATCTTATCCAACGTCACGCCGCGCAGTTGCGGAGGCT
GCATCTGTGTCTCGTCGTACGATGCAGACTGGCCGTCGTTGTTCGTGAAGACGCCGACTT
GATACACTGCACCAGCGCCCTCAGCTTCGTTCTCGTCCTTCGCGCCTAGCTGGAGGATGC
GATACTTGGTCTGCGACACCCATTCGAAGTCCGTGCTGCGACGGAAACCGCTTTCGTCCA
GGACCACAAGGTTCAGTCGCGCTTCACCTTCGTCCGCCTCGCCGTCGTCCCAGTTCCGAA
TGGACTCAGCCACGTACATGGCGATATAAGGCATCGGATTGGTCGGATCGGGATTCACGG
GCAGGTCAAGCAGCAGACCAAGGCGCCCGGTGACAAGCTGTTCTTCGTTGATGCGCCGCA
GCAGCAGCTCCAGGGGCTCACCGTAGGCGGTAGCCTTGTCGCGGAGCGCTTCCATGGTAG
CAGGCAGCTCGATCGTCGGGGTCTTCTGCCACATGAGGCCGATGTAGGCTTCCACACCTT
CTTTCACGTAGTCGTGGAACACCGCGCGGAGCTTGTATGCGTCATAGGCCTCGCGCCCAG
GCTTGCCCGAATCCATGCCGTCCAGTCGCATGCCCTTGGTCGGCGGCAGGTACGTCTCGC
CCTTCGCCTTTACCGCGCGCTCACCTTTGTAGAGGTCGCGCATGGTGACCCAATCCTCTT
TGAATTCCGAATACTTCGGATGAACCGAATCAAGTGCCATTTTGCGCCTCCGTCAATAAG
TGCCCGTAACTGTACCCGATTTCGTAATGATTCGTCCAGCGGGCCAGTGCCAGTCAATAA
AATACCCGATCGCTGTGGTGATATGCTGATACTGGTTCTTCTGATCCTCTTGGAAAGACG
AACCTGCCTGGAGCTGCACAGTGCTCAACCCCTTGTGGCACCACAGCGCAGTCTGAGGAT
TCACCTTCAACCTCACTTCGCCTTTCGCATTGCAGATCATCGCGCGCACAGCGTTCTGGC
GGTCCTTGATAGCAGGGTGCGCAGGACGCACGCGCCGCTCGAACTTCCAGCCGTGCAAGC
GTAGCACGTCTTCGATCTCGGTGTAGTCGGATTTGTGCCCGTGCTTCTCACCCGCGCGAC
CTGCCGGGTCGCCGTAGATATAGACTGTCTTGTTCTTGTGTTCCTTGTACTTCTCGACGA
ACTCGATTGCCGACTGACGGCTCACCGCACTCTCAAGAACGATCTCATCTAGGAAGAAGG
GAACTCCATTGCGGATGACCACAATAGCCGAGCTCAGCGGGGTGAAGTTCTGGTCGTGCG
TCCAGTGCAGCGCTTCGTGAGGGAGGATCCGCTCGGTTGTGAAATTCTCCTTGGAGTAGT
CCTCATAGATACGACCGCTGGCGGTCTCGAAGCTCGCTCGATATTCCTGATTGTACTGCT
TCCGCGACATGGTGCGCTTCGCAGCTTCAATGACGTCTGGCGGGAGAATCTCTTCCGACG
TCCAGTGGAACAGCGCCCAGTCGGGGTCGTTGGCGGTGCGTGCATATTCCGCCATGTCAT
AATAGTGGTTCAGACCGTCGGGCACGCCAATGAACCAGCACCAGGCCCGATAGTACGGGC
GCGTCGGATTCACGGTGTTGAGCGCGGGCATGATGTTCGCTTGCAGCGCCTCACCCTTCA
CGTCCGCAATTTCGTCGATCACACCGCCTGTCCAGTTGATACCTTCAATCCGCTGGGGCT
GGTCCAGACCGATGATGTGAATCTCGGTTCCGTTGGGCAGGTAGATCTTGAGTTCGGACT
CGCTAGGCTTCTTGGGGTGCATGCACGACAGCGTCAGCGCCTTGAGGTCGTCCCACCAGA
TCTTCTTGGCCTGGTTGTATGTGGGCGCGGCTGCGAAATACTTCTCGCCCGGATACCACA
TCGCCTGACGTGACAGAAAACGCTTGGCGCGTTCCGTCTTGCCTGAGCGTCGTCCTGCGG
GCACAATGGGGAAGCGCACACCGTCTTCAACGGCACGCAACAGCCGAAGCTGAACCTCGT
GGTCCTTCAGCTGATACCAGCGCGCCATCTGGCGTTCTAGAATGATCTTGCTCATACAGG
CGCCCTCGTCGCAAACTCGCGGAAGGCGTCAATGAGAGCTTGCTCACCTTCTTCACCGCC
GTCAGGACGGTCAAGGCCGTATATGCTGGCCAGCTTGGCGGCAGCTTGTACGCGGGAAGC
GTAGGGGCCATTCTGTGCAGCCTGGCGCAGCACGCTCAGGATCAGGGCACGGTCTTCCTT
AGCTTGGGCGTCTTCGTTTTCAGGGGTCGCGCGTTGCAGTTCAGTGATCCGGCGTTGGAC
ATAAGCCTCGCCCATGAACTTCTTCGCGTACTCCACGGCGAAAGCAGCCTGAAACCCTAC
GCGAAGGCAAGCCTGGAAAGGATCGAAATCCTTTATGTACTCCTGAACGAATAGATCACG
AAGGGCCTTTTCTTGACCGGACGGTTCCGGCTCCATGAGCCGAGGATCCAGATATAGACC
CGATGTGTTCTCAGCCATGTGGGAACTCCAATCGGATAGTTAAGGCGGATCACCCGCCCA
ATTTGATCGGAGTATAGCGATTCTGAGGCCGGATACGCAACCAGAATATCAGGGCGAGAT
TTTGCGGCAAGCCGGCAATTCTGCAGGCTTTTGGCCGGTGTTCCACTCAATAAGGCGGGC
CATTTTGGACGCGCATTCGCAGAAGGACGTTATCCAGTCCTGACGCGAATCAATGAGGTC
TTTTGTCACTACCAGCTGACGATCCGCTGCCTGCTGGCACTGCTGCACCAGCCCCTCGGG
AGGTACTGCCCGTTCCACCTTTGTCACTACCCGAATTTCCGGTGTCGCAGGTGTTGTTGA
GCAAGCAGGCAAGATCAGGAGGGATGCGCTGATTAAGATAATTCCGAACAGTTTCATTTG
ACTTCTCCAAGGTTGCGAGACGGTTACGCGCGCGAGCATCGTTGTCCGCCAGCACCTTGT
AATCGGTCAGCAGCCCCGTGAGGGCCTGTGCATCCTGGAGACGAAGTTCTTTCAGCTCGC
CGATGGTTTCCTGCTGCTGCTGATTGACCGATTCCACTGCGCCCAGGCGCTGCTGAACTG
TCTGAAGCTCGTTCTGTGTGGTCAGCAGCGATTTCTCCGTGCGTTCCTTGGATAACCACA
TGCTGAAGGTGAAGCCGGCCGCAGCCACTATCACCGCGATCAAGACATACTCGATGACGA
GCCTGGTCTTGTTCTTGAAGAAGCCCAAGGCACTTGCGAGCCAGGGCAGCTTAGTCAGCA
GAAGGGTGATCATCGGCGCGCTCCCGGATGGGTTCGGAGGGTTGCTTCTCGATACCGAGC
TTCTTCAGCACGATGGACTCGAGCATGCGGATCGTGGCGTTAGCCCCCAGCCAACCTGAC
ACGCCAACGATCACACCCGTCCATTGCTCGGACAGGTTCATCGCCTGGCACACCAGCAAC
ATCAGCAGGCCAACGAAACCAGCCGCGCCACCCTCCAGAGCTGCGCGACTCCACTTGATT
TTCTGACGGCTGTCGATGGTTCGCATGAGATGCCCCATCATACCTCCGAACATGGCGAAG
AGCCCGTAGGCCAACGCCTTAATCCACCAGCTATTCCACGGATCTTCCGGCAACATATTT
GGACCCCTATTCGATTGATTTCGACAGAGGCCCATGCCCTGTTTCGCGGATAGTACCACA
TGCGAAACGCATCAAGCTACAGGAATCAAACTACAGCTCAAGCGCGAACCGATCCAGCAT
GCCTTTATCAAACTTGGCGCGGACCTTGCTCGCTGCTTCAGCCTTCGTGATCTTCCCGTC
GCGATTCGCATCCAGACCAGCGTTCTGGCGGTAACTGATCTTCGGATCCAGGAACAGAAC
GGAATCCTCTGCCTGCCCCACGAACTTGGGCATCAGGATCGCCATATACATGTCGCTGAG
CGTACCGATGCGGGAAGCATAGGGGCGGAAATACTTCTGAACGTAATCCAGCTGATCCAC
AGCGCTCATGGCTGCCAGCTCCGTGGTACTGGTGCCCAGGCCTTGCGCAGTGCTGGGCAT
GAATTGGATGAGTCCGGTGGCGCCGCTGCCTGCTGCATTGCGCACGCTGGGGCTGAAGGT
CTCGCCGGATTCGAACGCCATGCAGGACATGAGCCAGCTGGCATGCGTTTCACGGTTCCA
CTTGAAGCCATCGCAGATGGCAAACACTTTGTCACGGAAGGCCTGATCGACCTTTTTGCC
CCAGGCAAGTTTCATATCCAGCTCCTGATGAGGTTGATCGCGAGGATAATCACTGCCCAG
AGTGGAATGCTCAGCAAGACGCCATTCACTACACCGCGAGCCGCATCAAGATCGTCTTCC
ATTTGTCAGACCCTCACGAAAACTGGCCCGAGTGTAGCAGGCTCGGGCCAGTAATAGCAA
GTTGATCAGCTTAGTGGGTGAGCCAATACCCCAGCGCAGCGGCAGCAGCCAGCACACACA
GCGCGATCGCATGCGCGCCAATACCCATGGGCCACCGCCAGTACGCGCGCGGCTGCTGCG
GCCCCTGCTCGTCGCGCAGTGCTGCCTCCAGTTCCTTGGCCAGCAACTTAGCTCGCTTGC
GCTCCTCCCGCGCCTTGAGGCGACGCTCGTGCTCCTCCATAATTGCCTTAGCGGTCTCTG
GTGACATTGGAAATCGTGGAATCACTTTGCGTCTCCTATTGGCGCGCTAGTGCCTACGCG
CAGATGGTTCTTCATATCCACAGGAATCACATCGAACCCCATGTACTTCCTTGAGTCCGG
TCCGGTTACTTGCAGATCTGCCTGGTGTGCGTTGGCGCGAATGAGGGCCCATTCCTCCCG
ACCCACATACAGCAGGTCAGGCCAATTGTTCCCGTCACGCCCGAGCATCTTTTCCTGGGC
TGTAGCCTGGCGCCACACCTCGTTTTGAATGTCTTGATATTTCATGCTGCCCTCACCACT
ACGCCCGAACCGACGTAACCGACGCGGATCTTAACAGGTCCGCGCTCCATGACGATGTGG
GGCTCTCGCTGCACAATCTCGAAACCCTCACGCTTCTTTGCCTCCACTGCTTCGCGGAGC
TCACTCTGCAATCGCTTTTCCATAATGATCAACCTTCTTGCCGAAGAAATAGGTGCCCAC
ACGCAGACGACCGTCGTCCAGCTTCACGATGTTTGTGTAGTAGTCGCCTCCAAGACGAAT
CCACTGATTGGAATCATCGATCTTGGTCTGAACCTCGATGCGACAGGAGGTGGCCTGGAG
CTTCACGCGCATACGACGCGAGGTGATCTCCTTGGTGACCGGATTGCGGCGTTGAACGTC
TTTCTGGTAGTGCCCGAAGCGGTCGGCTTCCCATCCGCATTCCTTGAGCACATCGATCAG
CTCTTGCTGTTTCTTCGCCATTGTGGTCTTGCCGCAACGACGCCCGAACATCACGATTTC
CATGGTGTTCTCCTTTGGAATTGAGTACATGGGCACAGTATAAACGGGCGTTACAGGATG
CGCAAGCCCGTTTATATGGTTTCCCTAGTCCCGGAGAAGCATGAGCTGGCGCTGGAGCAT
CTGCACCGACGCCAGGCTGTCGAGCATCAGATCCAGCAGTGCATGGAGGTTCTGCCGGTT
GAGGTCGGCTTCCTGCCCGTGGTTATCGAGACAGGCGGCCTGGTACATGTGCAGCGCTGC
ATCGTAGCGTCGCTGATATACGGTGTTCTTCAGGACTTCTTCCTGCATCGCCTGCAGAAG
CTGTGCTCGATCCATCATTTCGCCACCTTACGAATGATCTCGACCTTGCCATGCACTGTC
TCAGTCTCGACCTTGCCAGTCTTGTGGTTCTTGACCGGAATCCCGAAGACTGCCTGCTTA
TAGCGAATGACGTAGCCATGTTCGACGTCCGCCACAGTGCAACGGGTCTGACGCTTACCA
TCCAACAGCACGTCGAACATCTTGCAATAATCCATCGAGTTCTCGCTGATGGACAAGCGC
TTCGGGTGCATGTTCATCATGCCTCCACAGGTTTCCAGGCACCCGTCTGGAGCATGCGAC
GCATCCAGTTATAGGCGAAGGTCTCGAAGTCCTCGTGGCTGATTTCACAGCGCATAGGGG
AGTGAGCCTGACCACACTGATAGGCCACGTTCGCCTCGATCAGGATGCGCCACTTGCGCT
TGCCATTCTGGCGGAACACGACGATCGGGCGCCCGCCGAACTGCTGCGCAGCGGTGACGC
ACTGCTTCCACCATGCGTTCAGATCCAGTTGCTCCTGACGCTTGACTTCCAGGCACATGA
GCACGGGGTTCGTGATGTCGCTGCCGCCGACTGCGGACTGGTTCTGGTTGCGCTGGAAGA
TCGGCTTGGTGGGATAGGGCAAGCCCTCGTCCTTGAGCGCACGGTAGGCAATGCCGTTGA
GCCAGTCGCAGACCTCACGCTCCCCGCTTTGCCCCTTCTGTCTGATGTTTATCGACATCT
AGTTTTCCTCGTTATTGAACAGGCGTACAGTAAAACAGGGCTCACCCGTTCATACAAGAT
GAGCCCTGCAAGTTATCACGGAATCTCGCAGGCGCCGGACGTGCAGGCCAAGGTCTGCAT
CGCCACGGTGTTGTCCGTCTTCTCGTAGGTGGCCAGATCACGCCAATCGACTTCCGGAAC
AGGGTGTTCCTTCTGCCACTGAGCGAACTCTTCAGGCGTCATGTCCTGATAGGGCGCCTG
CTGATAGGTGTGATCGGTATGTGGAAGGAAGCTGATGCCCGACACCTCGTCGAAGTGCTC
ATAGACCCAGGCGCCTACCGACAGCCATTCCTCGTCACGCACGGTGACAGTGACGGAAGG
CTTGTGCTCACACCACTCACGTTGATACAGTAACCACAGTTCCAGGGCCTGCATCGCAGT
CACCTGATCACGGGTCACAGCACCATCTGGCGCGGACATGGCGAAGCTCACGACGGCCGT
GCTGTCCGGACGCATGACATCGTCTTCGGTGACGAAGCCCTTGTCCTTCATGAACTGGTA
GAGCGGATCCTTCTTATCGACGCGCACCGTGCGGATATAGTTCCGATTGTGGCGTGCGTG
GATACCGCTGGCGGTGTTGCAGAGCTGGCTCACGGTGCCCGACGGCTTGACGCAAGTGAT
GGCCGCAGAAGGCTTGATGCCCAGAGCTTCCGCCCACTCGGCATTCACGAGACGTGCGTG
GTCACGCATGCTGTTCAGGGTGTGCGTGAGCTTGTCGGCGCCCAGCTGGCCGGAGGTCAG
CGCGTTGTCCATGATGCCCGTCAAGCTGACGCCCAGCAGAGCTTCCTCGGCAGTGTTGCG
CGCCCAGGCTTCCGACACGAACTTGAAGTCAGTGAGGGTGGCTTGCAGGGTGCCCAGGAT
GGTGGCGATCTCGACCTTCTCCATCAACGTGACCATCGTGTCGTCTTCACGGATCACGAC
TTCGGACAGGTTGCAGAGTTGCTTGTCGCGCAGGATGATCTCGGAGCACGGGTTGCAGCC
GTAGGACAGTTCCTTGGAGCGGCGACCCCAGCGCGAAGCCTGTGCAGCAGCGGCGCGACG
GTTGAAGATACCGCGCTCACCGGACTTGGACTTCACGAGCGACAGCCACTCCTCCATGAA
GATCTCGGCACTCGGCTTCTCGGTGTAGGCCACGGAGTTGTTCGCCAGGCCGCGTTGCGG
GTTGTCGTTCCACCACTGGCCAGCCTTTGCGTCACGCATGCGCTGATCCGACAGGTTCGA
CAGACTGATGAGCGCACTGCGACGCACACCGCCCACCACGACGATCTCACCGATCATGCA
CAGCAGGTCATGAACCTCGATGCTGTTCAGCTTGCGACCCTTCGCGCGCTGGAAGGTATC
GACGGTGAATTTGAACAGACGCTTGAGCGGATCCGGGCCGGACGCACGACCGCCGAAGAT
CTTGAGGCGGGCACCAGCGGGCCGGACCTTGCTGTAATCGATCTTCGGGATGTCACCGTT
CCACAGATGCCCGAGGAGCGCATGGTAGGCCTTCGCCCAGCCTTCCTTCGAGTCCGCCAC
GACGATGGTGTCGTCGGTGTTGAGCAGTTCGTCAGGTACGGCGGGCAGCTTGGTGACTTC
CTGGCGCTCGCAGCTGAACCCGACACCAGTGCCGCACATGAGGATATAGAGGGCCTCAGC
GAACGAACGCTTCGTGTTGATCGCCAGATAGGAACAGTTGAACCCAGCGAGGTTCTCACG
CTCCAGCGCCTCACCGGCAGTCATCAGGGCACGCATAGAAGGCATGACCTTGAGCTCGAG
GATCGCGTTCTTCACCTTGCCCTGGAGCACCTCGCTGAACTGGTGCTTCGTGCGCTTGTC
GAAGAAGTCGATATAGCGCTGGACTGTTTCCTCCCAAGTCTCGCGCCGCCCCAAGTCATC
GCGCCAACGTGAATAGCGCGAGATGTGAATGTATTGTTGATAGAGATCCATCAAGGTTTT
CCTCTTGCGGTTGTGAAAGCGGTATGGTACGCGATCGATCTGCGCTGTCACAATTCCTAT
TTGCCGCCCAGCTCACGAGTGATCGCGTACTCCTTGGCGCTCCTGCACCCGCCATGATGA
CAGATGCTTCCACCCTGCCCCATCTGCGAGCCACAGCAGCACAGGTCAGAATCGATCTTG
CGATAGCGCCACAGTGCAAGCCAGCGCCTCCAGAACTTCGCCTTGTAGAGGGCAGTCGAT
GCCGCTGTGCTACCGGACACCGCCAGGAACACTTCGAGGACGAGCCATGCAGCCTGTGCC
CAAGACAGATTCACCCCATGTAACCACACAGCTAGGCCCACAGCCGACGCAATGCAGGTC
AGCGCCACTACCGCACTGATGGCGAACAACGGCGCGACGCTGCGCTCCTGCTGCGCCTCT
ACCCATGGACACTCCGACTGATCGTGCCCTACCCCTCCGCACAGGATGCACATCTTCATT
CTTCGTCCCCTGCGGATGCCAGCTCGATCATGAGGCGGCGGGTGACGTGCTTGCCCACAG
TGAGGCCACTGAACTTAGGCTGCATCTCAATATCCACGAGCAGCTTGTGGCGCTGCCAGT
TGAAGGGCACGCGCCCGTCCATGAGGTAGTCCCACAGGTCGCAGCACTTCTCAGGGTCGA
ACAGATCCGTGTGAGGCACATGAAGGCCCATCACGGACTCGAGCTTGTTCTGGGCGTCCA
GGAGGTCGAGCTCGGGCAGTCCGATCTGTCGCATAGAGTCTGCGATCGCTGCGCCGTCAC
GGTGCAGAAGCACCTTCCGAGCGGGGTGCTTGTTCAGCCAGTCCGTCCAGCGCCAGATGC
CAGTGCAGGACACGCCCACGGGCACAGAGGCCTTGAGCTTCTCGTCCCATTCGGAATAGT
GAGTGTGATAGAGCGGGTCGTGGTGGCACAGCACCGCATCCGTCGTGAATAGGTTGGCCG
CCCAGGTTGTACCAGACCGGGGCAAGCCGATAATCATGAAGTCGAGCATGGCGTGTCCTT
CCTGTTGAAGTATCGACGAATGACGTAGCTCCGGGCAACGGACAGCGCGGTCATCCACGC
CGTGATCTTGAAGTTATTCTCCCACAGCATCGGAATGTCGTGGAACCAGCAGATCGCAGC
AGTGGCAAGCATAGCGATGACCAGGCCAACCAGTGTATTGACGACAGCCTCGATGAACGA
ACTTCGCTTTGATTGTCCGTTCCACGGGCAGTTCGGCGCGGTGTGACCTTCGGCACCGCA
TAGCGTGCAGACCTTCTTCATATGATGGGCTTTCCGTTGTGGCGATACCAGTGCGGTTTC
AGAGCGATCTTCTCTTGGATACGTTGTCGCACCAGTGCAAGCGCTTCGGGCGTCGGATCG
TAGTCCTTGCGGAAGCGCTCGTCCAGCGCATCGAACACACCGTCAGGATCGAGCAGACTG
TCGCGGTTGAAGTTGATGCCCCGACGCTCCAGCTCCGCCCTCAGCAGGGCATAGCGCTTT
CGCAGATACAGGGCTTTGTCGTAGAAGAAGCTGACGTGTCCGGTGTTGAGCGTGTAGGCC
TGGGGCACAGCTTCGAGCACAATAGCCTGACCCATAAGGGCCTGGACGTATGCGTCGCGG
TAGCCGTGCGCCTTAACGTCACGCGCCTCTGCTGCCCGCAGACTGCGGCGCAAGCTCTTG
GGCACCATCTTAATTTCGCGGAACTCCGCGAACAGGTGCTGATCCATCAGTTCTTCGGGT
GGAACAAGGTTAATTCGAGTCATCGCCAATCCACTTTCTTCAATCTAGTTGAGGGGTTCG
GAATCGTCATTGCGTAGTCCAGGCACACCAGCCGCCCGTCGAGCATGCCGAAGTTCTCAG
GCTTCAGGTCACTCAGGAACGCTGGCACTTGCTCAGGTAGCACCGACCGATCTACTATAG
GCGAAGCCTTGCGCTGTAGCAAGATGCGCCCGTCTGGTGACAGGTATTCACACGGAGCGA
GCCAGTCCGCCACCTTCTTGTAGTGCTCGTGATCGTTCCAGAATCGCATCTCGAGCACGT
TGGCGAAGTAACGCCAGTTCTCTTCAGGCTCCACCTTCACGACTAGATCGTCGCGCAGGC
GACATTCGAAAACCTTCCGATGGATACCCTCGCCGAGCAGCTTGCCACACAGCAGGTTGA
AGGCGTCTTCGTATGTCGCACCGTTCATCAGCTGCCCGCCTGCTTGCGCATCACGACACG
GAACTCTTCCTCCGCATCGGAATTGACCCAGGCTTCCTCAGGGCTGCGCCACGCGCACCC
GTTCGGACTGGCGCCGTCCGCTTCACCGCGCATCTGGAACCCTTCCAGGAACGCCTTGCG
GATGCGGTGCTCTTCGGATTCGATGTTCGCCTTCAAGGCTTGCAGGTAGTTGGCGAGGTC
AAGAACTTCTTCGATGGCATGCTGCACCCATTGCCCGAGGTTCAGATCTTCGCGTTCAGT
CGTGACACCATACTTCTTGAGTCCGACGACGCTGCGATCCAGGAGCATCCGGCGGTTACG
CTCGACGTTGGCATCGGGTGAGGCGGTGACCACAGTGACGCCCTTCAGCAGCTCTTCCAG
CTTGGTGGCGGTCACCGGAGTGATATCACCGCTCGTCACTGCATCCTGAAGGTACTCCAG
GAACGACAGCACGCCTGCGCACTGCACACCGACGCGCACGGACAGACTTCCGACTTGTTG
ATTCATGCTTACTCCTTGAACGGGTTGGCGCCCGACACTGCGATGGGCGGATGGGTAGTG
CCCCAGGCGTGGAGCCAGACGTGTCCGGTTCTCATGACCTCTGCGAGCTCGTCTGTGCTG
AGTTTCCACTTGGAGATGACGTGAGGCTTGCCTTCGGCGTCCTCATAGCGATGGATGGGC
AGGTCAATGACCTTGCCTGATGCGCGGTCCTCGGGCGTGCCGAACAGCACCGCATTCGCT
TCCGGAAAGGACACAGGCCAAGTCGGATCCGATTCCAGCATGGCATCCAGCGCCTGAATC
TCTTCGGTGATGGAGTCGTCCTTGTCGGGCAGGTTCTCGGAATGCCACTTCAGGAACTGC
CGATACTTCTGAATCTCGCGTTGGATATAGACAGGGTTGCCGTCGAACCCGTTGAGCTGA
AAGAAGGCGCGTGCTGCTGCGAACAGGTCGCACAGCTCCAGAGCGATACGCTCGTCCAGA
TCACCTTTGCCATCCCAATGCTGCACCGCGCCACCAGTGCCCATGATCTTGCCCGCGATT
TGCAAGACCTCACCACATTCTTCGACTAGCTTACTCAGTCCAGGCCACGTGCGTGATCCA
AGGCTGAAGCTACCGTCGCCAGGCGTTCTGAAATCGTTCATACGATTCTCCAATAGTCAA
GAGGAAACGCGGCCCAGGCACCAGTCCCAGGCCGCGCACCATATTCGTAGGCGTGTGGGG
CCTACCCGCTTGCCCGCCCACCCACATGGGCACGCACACGGTTTCGCTTCGCTCTATCGT
CCTTGCGGGTAAGGCCCATATCCGGGCTCCGACAGAGCTAGGCTCGGCGGTTTCTGTTGC
GTCCGGTGAATTATGTGGTCTCCTGCCCCGCCGGAAGGGGAGTCCCAGGCCTCGCAGATT
CATCCACCGTGTATAGTGGAGCTCACTGCTGCGCCGTGGGCTGTTACTCGCTGCATACTA
TAACGCCCACCGTGCCGGAGCACAAGCGGGCGTTTTGGATATCTTGCAAGCGCTGAGCAC
CGTGATGACCACAGTGCCACGCTCGTCAGTAGGTGAAGAAGTCTTTGCAGAAGCCGAGGT
ATCCGCCCGTCGAGTTGCTGCGGCGCAGGAGCTCGGACAGGTTAATGCAGCCCACCCAGC
GACCCCAGGCTTCCACGTAGAACACCACATAGTCAGCCGACGGAGCGTCTTCCCGACCTT
GCTTGTCGAAGTAGGTCGCAGCAGCCTGGGCCATCTTAGCGGTAGCCTTCTCCGCTGCTG
CCTGGGTGCCGTAGTTCTTGCACGGGTTCTTATTGGTCGAGCGATAGTCTTCGATGCGTG
CGGTCAGTTCGGTGATGATATTTGCCATGTCAGTTCTCCTAGTGCGTTGCGGTATGGTGT
TAATGTAGGACGAACTTCAGACCTATGCAAGCGACTTTTGCAAATCTTTTGGACCTAACC
GAACACATTAACACGCAAGCCGTGAGCCATAGCTTATAGGTCGGCGTTTCAGCAAACAGG
TCTGGCTGATCGCGCACGCGCCCGCATTATGCGGGATTGCTCTATACAATATAACAAATA
AACATATTAACACTTATATAGAGTAGGCTTATCTAAAGGAGGGGGATCCTCACCCCCGGC
TGCGCGTATTGTATGCCACCCGAGGTTGTTTCGCCGCCCCGCTAAGCCCCTGGCCTCCTT
TATTTCTTGCAATACAAGGCCCCATGCCTTAGCTTATGCACTGTTCAATTCATTCCAGGA
GTTTTTATCATGGCTCAACTTGATTTTGACGCTACCGCGTTCCCCCAGCCTGCGCAGCAA
GACCCGCTCAGTTTCTTCAACCAGATTGACAACAATGATCCGGATCCGTCGCTGGTGCTG
CAATACCTGCACGCGCTACACACGCAAGCGCTGGCGCAGGGCATGGAGTGCGTATCGGTG
GAACAGTTCTACACCGACCAGGCCATGGAGCAGTGGCGCATCCTTTGGGCAGAGCGTCCG
AAAGACGTTCAATTCCCTGCCCATGTGGATCTGCTTCAGATCAAGAGCATTCTGTTCAAT
GCGCTGACGCCGTTCAAGTGCAATGCGACAGGCATGGAATGCTTCAGCATGCTTGACCGT
GTGAAGCGCGCTCACCAGATTCAGTCCGTGTGGTTAGCTGAGCAGAATCACCAGGAGGAG
ACACCAGCGCAAAAGCGCGCACGTCAGAACCGTGAAGCTCAGCGTCGCTTGGCATTGCGT
TCGAAGGACGATGGAAGCCCGGAGTCGGAGCACGCCAAAGCGCTCAAGGCTGCCTATGAC
GCTTACCTGCTCGCGTGCAGGCAGCGCAAGGAAGCGGAAGCGCAGTGGGCTCAGTACGTT
GCAACCAAGAAAGCGGAGTACGAAGCTATCAAGGCGCAGAAACCGCTCAACTAAACATGT
GTTGCGATAACACAGCGAACGCGCTACACTGCTGAGCCATTCGGGTGTCGCTTTCGCGCT
CCTAGTTTGCCGGGGAATCAATAATGACGGAAGTTGAGAAGACTGCTCAGCAGGAGTTTG
ACGAGATCTATATCACGTCAAGCGAAATCGGCCAAGAGCTTGGTGTGTGCCGAGCCACTC
TAGTTAATGCGCGCCGTCGGGGCCTGCTGCCCGATCCGATTGTCGTTAATGGCGCGCAGA
TCTTTGTCTGGAAGCGTGATGTCGTTCGCCCTTACCTTGACGCATGGAAGCTCATGCTCC
AAGCGCGGAGGGGCGAGCTGGTATGACCACACGCGCTCAAGCATGGGCGCGCCTTCCAGC
TGAGCTCAAGTACCTGCGCCAGTGGTGTGTTGCTGGTGCTAACAAGGCTCCGCTGTCGGT
CGGCCCTGACGGAAAACTATTCAACGCCTCTGTGACTCAACCGTCGCAGTGGATGGACTT
CAATACGGCTGCCCAGGTCGCATTCGCTAACGGGTACGACATCGGCTTCGTTCTGCATGA
GTCGGACCCTTACTCGTGCATCGACTTGGACGTCAAGGACGCTCAGAACTGCGCAGATAA
GCCCGAGCTCTGGACGACCCCCGAACAGTTCGATCTGTTCTATCGCATTATGCTGGGCTT
CGACAGCTATGCGGAAGCGTCGCGCAGCGGGAAGGGCTTGCATATATGGGTCCGGGGCAA
CATCGGGAAGGGTGTGCGCCGCGATGGGGTGGAGATCTACTCGCAAGAGCGGTTTATCAT
CTGCACCGGCAGCGTCGTCCAGGACAAGCCCGTTCGCGACTGTCAGCCTATGCTGCTCAA
TATGGCGTCGCAGATGCGGCCCAAGGAACAGGCGGTTGATTTCGCGCTGCAAGAGCTGGA
ACAGGACGAAGACGACTGGTCGGTTCTGGTGCGCGCTGTCAATGCAGGTAATGCGGACAA
GTTCTGCGGGCTGTGGCTGGGGCAATGGGAAATGATGGGATTCCCGAGCCAGTCTGAAGC
TGACCTCGCGCTCATGTCCATGCTCACGTTCTACAGCCCATCGAATGCACAGTGCAGGCG
CTTGTTCCGTGACTCGGCGCTGGGTAAGCGGGAGAAGGCAGTCAAGGACGATCGCTACAT
CAACCGCACGCTGGCCACAATCCGATCTCGTGAAGCACGCGAGAAGGCAGTCGATGCCAG
CGCACTGGTACAGGCGGCCGATACCCTGCTGGAAGCGCGCCAGGCTGCTATGCAAGAGAT
TCAGCGCTTGCAAGGGGGTATGCCCGCGGCTGGTGCCCCACAGCAGCGCACAGCGGTCCC
GCTGCACGTACAGGGGCACGGTGATCCGGTTCAGCTACCCCAGCCCACAGCGGTCGCCGC
TACGCTGGCAGGCCCCGTGTCCGCTACCGTCGTTGAAGCTGGAACAAAGGGTCTGCCTTG
GCCGCCTGGGTTCGCTGGGCGAATTGCGCAGTACATCTATCAGAGCGCACCGCGTCCAGT
CAAGGAGGTGGCGATCGTTGCATCGCTTGGGCTGCTGGCGGGCTTGTGTGGTAAAGCCTG
GCACATTCCGCAGTCCGGTCTGAACATGTATATCATTCTCGTCGCACGTTCTGCTATCGG
GAAGGAGGCGATGCACAGCGGGATCAGTTCGTTGATCCGCTCCTGCACCGCCAGCATGCC
TACGTTCCACAATTTCATTGACTTCACGGACTTTGCTTCGGGCCCTGCTCTTATCAAGGC
ATGTGCCAACAATCCGTCGTTCGTGAATGTGTCTGGTGAATGGGGGCGCAAGCTCAAACG
CTTGGCGCATGACGACCGAGATGGGCCGCTCAGCACACTGCGCACGCAGATGACGAACCT
CTACCAGAAGTCGGGCCCTCAGTCCATCGTTGGCGGTATCGGCTACTCGAACAAGGACAA
CAATATCGCATCGATCAGCGGTGTCGCCTACAGTATGATCGGCGAGACCACACCGAGCAC
GTTCTACGAAGCACTCACCGAATCGATGATGGAGGACGGGTTCCTGTCCCGCTTTCTGAT
CATCGAGTACGACGGTGACAGGCCTGCTTTGAACACGTCGCAGGTGATGCACCCTGACGC
GGCGCTCCAGGACGCACTCGTCAAGCTCGCATTCCAGGCGCAGAACTTGATCGGCGGGCA
GACGTCCCAGCCGCTTGGGCGCACCGAGGAAGCTGCTCAGATCATGTGGGCGTTCGAGCA
AGAGTGCGACAAGGAAATCAACAGCACCGACGACGAGTCCCGACGACAGATGTGGAACCG
AGCTGCGCTCAAGGTGCTGCGAATTGCGGGCTTGCTTGCGGTAGCCGACAACTGGATCAA
CCCGTGTGTCACGAAGGAGCACCTGGACTGGGCACTGGACGTGATTCGCAGGGACATCCA
GATCATGAGTAGGCGCTTGGATACTGGTGACGTCGGTACAGGTGACACAGCGCGCGAACG
CAAGCTCATGTCTCTGCTGCGCGACTACCTTGCGAACCCGCTTGCAGCCAGTTACAAGGT
GCCTGAGGAGATGCGACAGAATAGCATCGTTCCGCGGAGCTATCTGCAGATCCGCACAGC
GCGCGTGGCGTCGTATTACAACCATCGCGCAGGGCAGAACGCGGCGCTAGACCTTGCTAT
CCAATCCGCTGTGAATAACGGCTACCTCATGGAAGTGGATAAGGCGAAGGTCGCAGAGGC
CTACAACTATCATGGCAAGGCGTACCGCGTGATCAAGCTGCCCGACTACGCTGCTCAAGC
GAAGAAATCCTAAGCCGATGCAAAATATCCAGAAAATGGGCTTGCCGGCTCGCGTGTGTT
GCGTTACATTAACGCTCATGCAGTTCAACCAGTAGATGGAGGTAAGATGAAACTCAATGT
GAAAGCTCTTCACCCGGATGCCATCGTGCCGCAGTATGCGACCGTCGGCGCCGCGTGCTT
CGACTTGCATGCGCTCATGGAGAAGCCTTGGAAAGAGGGCGAACAATCCTATGCGCAAGT
CAATCCGGGTACGCCCACCGTGATCCGTACTGGGCTGGCGTTTGAGATTCCCGACGGTTA
CGTGATGCTGATTTTCAGCCGCAGCGGCCACGGGTTCAAGAACGACATTCGCCTCGCCAA
CTGTGTCGGTGTCATCGACAGCGACTACCGGGGCGAGGTGCAGGTCAAGCTCACCAATGA
CAACAGCGACGGCAATTTCCTCACGGTGAAGCATGGTGACCGCATTGCGCAAGCGATGAT
CGTCCCTGTGGAGCGTGTGGAGCTGGTGCAAGTCGATGAGCTGTCTTCCACCGAGCGAGG
TGAAGGCGGGTTCGGCAGCACTGGCACCGAGCGCAAGTTCTTCGGCATGCCTCAGGAGTA
ACCACAGCCATGACGCTCATTCCCGAAAACCAAGTCAGCCAGGCCGATCTGGCCGAGTGG
TATAAGCTCCAAGAGGAGCTGAAGAAGGTCAAGGCGCGCGAGATGCTTCTCCGCACCAAG
ATCTTCGGGTACTTCTTCACCTCCCCCGTGGAAGGGACGAACAGCTATGATCTTCCGGAC
GGCTATGTGCTCAAGGGTAAGCACACGATCAACCGTGAAGTGGATCCTGGTGCGTTCCAG
GCGATGCGCGAACAGTTCTCCCAGGCCGGCATCCACCCCGACGCCATGGTGCAGTGGAAG
CCTTCGCTCAAGCTCAAGGAATACCGCGAGCTGACCGCCGAGCAGATCCAGCTCTTCGAC
CAGTGCCTGATCGTGAAGCCCGGTTCGCCTGCGCTGGAAATCGTGCTCCCGAAGAAACGT
GCGGCAGCGGGGGCACAAAATGCGGGTGACAATCATAGCTGATGCGTCGTTCTGTCCTGA
CACGAACGTCGCCGGTTATGGATATTGGATTGCATCCGAACGTGGCAAACAAGGTGGAGG
CGGTGAAATGAAGTGCCGTGTGGAGGGTAACATCGCAGCAGAGATGCAAGCGGTAGTGAA
TGCCCTCTACATCGCGCACCGCCTCCAGCTTGTGCAGGCAGAGGACGAAGTTCTTATCCA
GACTGACTGCATGGCTGCAATTGATGCGTTCGAGGGTAAGCGGAGGAATCTGCCTGCCCA
GGAATGGGAAGCGGTGAAGGTACTTCGCCGCCTGCGTTCCGACCATAGCCTGAGAGTCGT
GTTCCGGCATGTAAAGGGGCACACAGGCAAGAAGGAGGCGCGGTTCGTTACGAACCATCT
CTGTGACAAGCGCGCCAAAGAAGCAATGCGCCGAGCTAGGGCTCGGGTTCAACTTGGAGA
AGTGCAATGAATCAGAAACATCTGTTGGCGCTGCTGCAAAGCGGCTACACCACGATCGAA
GTCGTGTTCAACGACAGCATGCAAGACAGCCTGGGCTCGGGTCGTACCAAGTCCAAGGCC
TACACCTACAAGGCCCGCCTGGATGACAACATCGAAGTCGGCGATCATGTGGTCGTGGAT
AGCCCGAGCAAGGGCCTCACCGTTGTCGAGGTGACTGCTGTTCATAAAACGGCGAAGATC
GACCTGGATGCCCCCTTCACTTACAAGTGGATCGTCCAGAAGGTTGATCGCACCCGTTAC
GACGAGACCAAGGCCAAGGAAGACGAATTCCTGGAAACCATGATGGAAGTCGAGCGTGTG
CATCAGCGCGAGCTGTTGCTGGCCAAGTTCAAAGATCACCTGCCGGAAGGTAGCGAGGCG
CGCAAGCTGTTCGAGAACGCCACGCAGAAGGTTCTGGAGGTGTCCAAGTGATCCCGAACT
ACAGTGGCAAGGGCGCTATCGCTCTGGACGTGCTGGATCACGGGTTCGTGGCCCTGCGCA
ACATGTCGGGCCCGAACCGTCGTCCGGATGCACCGTTCGACGCAGACGATATCGATCCGG
CACAGTCCGCTCGCATGTCGTTTGACCAGATGGACAGCGACCGCACCCGCGACGCGGATC
TGAAGCTGGCGGACTACCTGATGCGCAACTGGCACACCAGCCCGTTCGAGATGGTGGAAA
TCTGGCTGGAAATGAAGATGCCCATCTTCGTCGCTCGGCAGTTCGTGCGCCACCGCACCG
CTACCATCAACGAGGTCAGCGGTCGCTATGTGCAGCTTCCGGCGGAGTGGTACATCCCTG
AAGTCGTGGGCGGTAAGGCTGCCAACAAGAAGCAGGGGCAGGAAGACAACCTGGACAAGC
CTACTCAGGAATGGTTCCGTCGCGTGCTGGATAGTCAGTGCCGCCTGTCCTATGACCTGT
ATCAAGAAGCTCTGGAGCGCGGTGTCGCTGCTGAGCATGCGCGGACGCTGCTGCACCTCA
ACCACTACACGCACTGGCTCTGGAAGCAAGACCTGCATAACATGCTGAACTTCCTGCGCT
TGCGCGACCACAGCCACGCACAGATCGAGGCGCAGCGCTACGCCCAGGCAGTTGATCAGC
TGCTGCGCCAGTACCTGCCGAACAGCATGGCGCTCTATGACAAGTATCGTCGTCTCGGAG
GTTAAGATGAAGCCCATGCTCGCGGTGGAAGCACCGAAAGAAATCAAGTTCCCTGTCTAT
GCCAGCGCCAAGCTGGACGGCATTCGCTCCGTCATCAATGAAGACATGGCAATGTCGCGC
ACCATGAAGCCCATCCCGAACGGGTTCGTCCAGGACTACCTCGGGCAAGCCCTGTTCAAC
GGGCTGGACGGTGAGCTCACGGTGGGCCCTGCAAACGACAAGAACGTGATGCAGGCAACG
ACCAGCGGCGTCATGTCCCGCGACGGAGAACCAGATTTCACCTTTTGGGTGTTCGACTTC
TTCACGGATGCCAAGATGCCCTACGGCGAGCGCTTGCGCCTGATGGAGCGTGCTTTCAAA
GACGGCGCGCTGGGTAACTACCCTCGGATCCGTTTGCTGAAGCAGACGCTCGTTCATAAC
GAGCAAGAGCTCCGCGCATTCGAGACCATCACTCTGGAGCAGGGCTTCGAAGGTGTGATG
ATCCGCGATCCGAAAGGCATCTACAAGTTCGGTCGCAGTACCGCGCGCGAAGGCTATCTG
CTCAAGGTGAAGCGCTTTGCCGACTCGGAAGCTGTGGTCATCGGTGTGGAAGAGCTCATG
CACAATGCCAACGAGGCAACCCTCGATGAGCTTGGATATACGAAGCGCAGTTCGCATCAG
GACGGCAAGGTGCCTATGGGCACGCTAGGCGCGTTGAAGGTGCGTGACGTGACCACAGGT
ATCGAGTTCAATATCGGTACTGGCTACACGGCAGCGCAGCGCGCGGAACTGTGGGCGATG
TGGCAGGCTGGTACGCTGGCTGGCAAGATTGCCAAGTACAAGCATTTCGAGGTTGGCGTG
AAGGAAGCCCCGCGCTTTCCCGTGTTTCTGGGCTTCCGCAACCCTCTTGACATGGGAGAA
GCGAAATGAAAATCAAGTCCGCTGCTCAAGTGCCGCCCATCATTGGCAATTTCTCTGACG
AGGAATTGATCCGGCACGTTGAGCACTACTATCCGAATCTGAACGGCCCGCTGCGCACGC
TGCTCACCCGCTTCGAATCCCGCGTGTATGGTACGCCGGAGCAAGTGCAGCTCGAGCACC
AGGCCGGCCAACTGCAAGACTTGCAGGAAGGCGACAACGAAGTGAGCTGCCCGCATTGCG
GTTCCGTGCTCCAGATTGACTTGGAGATCGAATCGTGAAGGAAGCCTACATCGACGCTCC
GCGGTTTCACAAGGCGTCGCTCAAGCTGATTGCACTCTGCGACGAGATCGTCACGGACTA
CAATGGGCAGGGCTACCGACTGACGATCCGCCAGCTTTACTACCAGCTCGTGGCGCGTGG
GCATATCGAGAACACAGTCCGCAGCTATGACAACATCGTCGCGCTGATGACGAATGCTCG
ACTCGCCGGTCTTATCGACTGGGACGCTATCGAAGACCGCACGCGCGGCATTCTGGAGCG
CTCGCACTGGTCCAGCGGTAGTGACATTCTGCAAGCGTGCGCGCAGAGTTATCACGAAGA
TCTGTGGTCGGATCAACCGAACCGTGTCATCGTCGTAGTTGAGAAGGAAGCGCTGGCAGG
CGTGCTGGAGCGCACTTGCAAGGAATGGGATGTTCCGCTCCTTCCTGCTCGCGGATATCC
TAGCGCCACCACGTTGCGCGAGCTGGCCAAGACCCGTATAATGCGGGCAAGCCAGGAAAT
TGTGGTCTTGCACCTTGGAGACCATGACCCGAGCGGCATCGACATGTCGCGCGACCTGCA
CGAACGCCTAGAGCTGTTCAGCAGGCACCGCGTGAGCATTGACTTCCGCAGGCTGGCCTT
GAACATGGATCAGGTTGAGGAGCAGAACCCTCCGCCGAACCCTGCCAAGGTCACTGACAG
CCGTTATGAGTCCTATCGGGCGCAGTACGGTGAGGAAAGCTGGGAGCTCGATGCGCTCAG
CCCGCAATACCTGCATCGTCTCGTGAGCAAAGAAGTTGAAAGTCTGGTAGAGTTCAGCCT
TTGGAACGAACGCTGCAAGGAAGTTGAAGAAGTACGGGGTCGGCTGCAAAAGCTCGCCGA
CGAGTTTGACAAAGGAGAAGGCTGATGGCTTTGCAATTCACCACAGCTGAGCAGGCGTCA
CAGCTCTCCGGAGTGAAAGCGCTGGTCTATGGTGGCGCGGGTATGGGTAAGACGGTTCTG
TCCGCCACCCTGCCTGCCCCCGTGCTGATCAGCGCCGAGTCCGGTGTGCTGTCGCTCCGC
AAGGCTAACCTGGAGCGCCTGTTTGGTGTGGGCAATCCGAACATCTGTTACAACATGCCC
ATCATCACCGTGACGAACGTGCAAGACCTGACGGACGCCTATGAGTGGTGTGCTCGAAGC
GCGGAAGCGAAGCAATTCGCAAGCGTCGGTCTGGACAGCATTTCCGAGATCGCTGAGGTC
GTGCTCAACAATGCCAAGCGGCAGGTCAAAGACCCGCGCCAGGCCTACGGTGAGCTGATC
GAGAAGATGGAGACGCTGATCCGCCTGTTCCGTGACCTGCCCGGGAAGAACGTCTATGTG
GCTGCGAAGATGGAGCCCATGAAGGACGAGATGACCGGTGTGGTGAAGTACGGCCCGTCG
ATGCCCGGTTCCAAGCTGGGTTCGAAGATCCCGTACTTCTTTGATGAAGTATTCCGCCTC
GGTGTCAATAAGACGCCGCAGGGCGAAACCTATCGATTCTTGCAGACTCAACCCGATCTG
CAGTTCGAAGCAAAGGATCGGAGTGGCGCACTCGCTCCGTTGGAACCTCCTTTCCTCAGT
GCGTGTTTTGCCAAAATCCTAGGAGCTTAAAAGCCATGGCACAATTGAACTTTGATGCAA
CCCAAGTCGCCCCGGATCTCGGTTTCGAGACCGTCCCCGCGGGCTGGTATAACGCGATGA
TCGACGAGTCGGAAATGAAGCCGACCAAGGACGGCTCCGGTGCTTACCTCCAGACCCGTT
TCAACATCATCGACGGCCAGTACGCCAACCGCAAGATCTACATGCGCCTGAACCTGCGCA
ACACCAATCCGGTGGCCCAGGAAATCGCCTACAAGCAACTGTCGGCGATCGCGCACGCTG
TCGGCGTTCTGCACGTCCAGGATTCCAGCCAGCTGCACGGCCTGCCCATGAAGATCAAGG
TGAAGCTCCGCAAGGACACCTCCGGCCAGTACGAGGACTCCAACGAGATCAGCTCGATCA
AGAACATCAACGAGCAGGTCGATATGGGCTCCCAGGCTGGTGCCGCCCCGGCGGGCTTCG
GCGGTGCGCCGTCTGGCGGCATGCCTCCGGGCTTCGGTGCTCCGCAGCAAGCTCCGGTTC
AGCAGCCTCAGCAACAGTGGCAGCAGCCGCAGAACTTCGGCCAGGCGCCCCAGCAGGCTC
CCGCACCGCAAGCCGCTCCGCAGCAGCCGTGGCAGCAAGCACCGGCCCAGCAACCGGCAC
CCCAGCCGCAACAGGCCCCGGCGCAAGCTGCACCGGCCCAGCAGCCCTGGCAGC

In another aspect, the invention provides an LPS-TLTL bacteriophage targeting Pseudomonas aeruginosa. In certain embodiments, LPS-TLTL binds a molecule of LPS (core). In certain embodiments, the LPS-TLTL bacteriophage has a genome comprising a nucleic acid sequence comprising SEQ ID NO: 4.

LPS-TLTL
(SEQ ID NO: 4)
TGGTCACCAATCGGCGGGTTATCCTGAGAGTTAGTTAGACCCTAGAGACTCGGTTGTTGA
ATGGTCACAGCAAACTATCGAGCCATTCCCAATGCCTGCCCTACCAGGGGCTTGTATCTC
AACCCCATCAGATGGCAGGGACGTGCCTATGTTAGTAGCAAGGCGGCATCCAGTGGTCCG
TCTGTATTTACGTGGTGGACCAATCCTATCGTAGGTACAGCTTTGTAGTTGGAAGGCTGC
GACAAGGCGCCCTATCTACACCGCCCCGGACCATCACGTCCGCTTACTGTCGAGGCTACG
CTAATCCCGAAGTCATGCGTTATGCCGTTGACTTGGGGGGCCATCTTACCCTTCCCTATC
CCTTGTCAACCCCCGATGCCTGCCGGTCTTATACTTGGGTGACAAGGCATTCCCCTTGGT
GGCAGCCCCGTTAGTGTCCGTAGACTCGGGGATGAGTGGCCCCTACGTGGAACGTGGTAG
CCGATAGGCCAGGTTCCTGGGGTTGTCAAGCTAGGGAGTTTCCCGTTGCCCTATCGAGGA
CACCTACATAGAAACGGCGCCATTAGGCCATATTCTTTGGGGTTGTCTACTGGTTTTCAT
ACAGTCTTAAGGACTATCGGTCCCCAGCAAACCCCCGGATATAGACAGTCTTTGATTAAC
ATAGGGTATCGTGGGATGTCAATAGGGGATCAAGGGGTTATCAAGGGATATTCCCCGATC
CACCCCTGAATACCCTAAGATTTTTCTTATGAATACCCTAGGGTCTATCCGATGAACGGT
AGCTCAGGGGTTGACCCTGGACTAACCTTATGGTATCCCTAGGGAGGCTAGGAGGCTATA
CCCTGAGGATAGGCCACCCCACTGTCTGGGGGGACTCCCTGGGGGGATATTCCCCAGATA
CACCGGAGGGATAAAATAGACCTTTGGGGTATCCCCGGTTATAGGCCCTGGATCCAAAGG
CAACCCCGATACCTGTTGGTATATTACTGGATAAAGGGCAATAGGCCAGGACATAGCAAA
CAGAGGTAGTCTAGGGTATACCTATGGAACAACCTCAGGGAGTACCTATAGAACCTGGAT
CAAGGGAACCCTATGCCATCACTTACCCCTTACCAGCAACCTACGAAGGGTAGTCTTAGG
GTATATCTATAGAACCCTATCATACCGTTCGTCGGGTAAATCATGTGATTATCAAAGAAA
TATCAAAAAAGTACTTGACAAGGTATCGAAATGGGGTATAATAGACCCATAGGCTCTTAA
GATCTTTCTTCCTTTGACTTACCTCTTACTAAGAAGGTTACCTTAAGGTTATCTTAAGAT
AGTCTTAGCGAACTCTTCCATTCCAGCAAAACCCCTATCAAATCCGGGTCTTGCGACCAA
AAATAAAAAAAAAGATGAAAATTTGACAAAATAGAAAAAAGTGTGATATAATAGTATTAT
AAGGTTGGAGAAATTAACCTTAAGGGTGCTTTAGAATTAATTGGGATGTAGCTCAGTTGG
TAGAGCAGGGAGCCGATAACTCTCAGGTCGTAGGTTCGAGCCCTACCGTCCCAACCATTA
TGGCCCATTAACTCAGCGGTTAGAGTGCCTCCCCTGTCTAGGGAGAAGCCGTCGGTTCGA
ATCCGACATGGGTCGCCATTAACTCCTCGTAGCTCAGTTGGTTAGAGTGCCTGTCTTGGA
CACAGGAGGTCGTAGGTTCGAGTCCTACCGGGTGACCATTTAGAGAGATCGGAGCAGTGT
ATGTCAGACGAAAAGGTTGTCTCGATTAGCTGCCCTTTCTGCTAAAGAAAAGCTAGACCT
CTATTGTGAGGCTCTGGCAGATGGCAGGGCGAATAAAACCCAAGCGTATAGCCGCTGGGT
TCTCCTAATCACGCTCAACGCAACGTTGCTGCATACCATCGAAAGCATGCCGAATACATC
AACGCCTTTATCTCTGAGCGCATCGGGAGTCATGTCCCCATGGCGCTTCGTGTGGTTGTA
GAGATTGCTAGCAACCCAGAAGAGAAAGGTGGATTCTCAAGGCAGCCCAGGATATCCTGA
CCGTGGAGGCTTCTGGAGCTAAGCAGAAACTGAGCTACTACTAAGAATGGGGAAGATATG
TCCGCAGAGGATCTAATCAGTGAAGTTCGACGAATTCCTCGACGAAGAGCCAGAGTTGGC
TAAAGTCATTTCTTTTCCCAACCTTAAGTACCTTTGATCTAATCAAGCGAAGAGAGGGGT
TGGTTCTAGCGGGTACAAAGACTCTCGGGAAGATAATCCGGCGGTTATGGCCACTTGCAA
CTCCCTGGGAAGACGGACCGATTAATTCTAGCACGAGCAGAGACTTGGCTAGAGAAAGAT
AGTAAGGCAGCCTATGATGCAAGCCTCCGCTAGTATTATCTCAGCTGCCTTTTTTGCACT
CCAGAGCTATTCGATGCCCTCTAGTTAGCGTCAACTTCCAGCTGGGTACAGCGTGGACGA
AGAAGTTCCCCGGGCGACGTGGAGCCTTCTGAGCCGGGAGTTCGACAGAGCAGCTTGGGA
GGCCGAGGATAGTGCCTGGGCTAAACAGAAACCCAGTGCGCGTAAGGGACCCTCCAACGG
CCCCTGTGGCGTGCATCAACAGTAGGTGGTTGGCGGTGGTCTAATGGATACCCAAGAGCG
GCTACGGAATCTAGTCAGGGAGCGGCTGAGCGGCAGAAGTATTCGCATGAACCAGTATAA
TTCTTATGGGTGGCAAGAGAAGTTTATCGCAGCCTCGAACTGCGCCCAGTTGCCGGCTAT
GACTGGTAACCGCTGTGGTAAAACCTACACCGGGCCTTTATCATGGCTCGCCACCTCACC
GGTCGCCACCCCGGAGTGGTGGACTGGTAGAGTATGCATCGTCCAGTGAACTGGCGGGGA
TCTCCACGGATACCACTCGGGATATTCTTCAGTCCGAACTACTAGGTGACTGGAACCCTG
AGGCTTTCGGAACGGGGATGATCCCCAAGGAAGATATCGTAGAGACGATTCGTAGGGAAG
GTAAACCCGGATGTGTGCAAGCAGTAGTTGTTAAGCATGCCTCCGGGGCCTCTCGTCCCT
TATCTTCAAGTCCTACGAAATGTCCCAGGACAAATTCATGGGTACTGCCATCGACGTTAT
CTGGCTCGATGAGGAATGCCCCAAAGATATTTATACCCAGTGTGTAACCCGAACGGCTAC
TACTGGGGTATTGTATATCTGACGTTTACCCCAGAGCATGGCCTCACGGAGATCGTGAAG
GACTTCCTCCAGGATCTTAAACCTGGTCAGTTCCTTGTCCATGCAAGCTGGGAAGACGCC
CCACACCTCAGTCCAGAAGTTAAAGAGCAGCTACTCGGTATACCTCCAGCAGAACGCAGG
ATGAGGGCCGAGGGTGTTCCTATGCTCGGATCTGGTGTAGTCTTCCCCATTCTGGAAGAG
AAGTTTGTATGTGAGCCTTCCAGATCCCCGATCACTTCCACAGGATCATCGGTATCGACC
TTGGGTTTGACCACCCTAACGCTATCGCCTGTGTTGCTTGGGACCCTGAGAAAGACAAGT
ATTACCTCTATGATGAGAGGAGTGAGAGTGGTGAGACCCTTGGGATGCACGCTGATGCTA
TCTACCTGAAGGGTGGTCACCAGATCCCGGTAGTTGTCCCCACGATGCCTTTAAGCACGA
TGGAGCAACCTCTGGTCGTAGATTCGTAGACCTTCTTAAAGATGACCACAACCTCAATGT
AGTGTATGAGCCCTTCAGTAACCCACCGGGTCCTGATGGTAAACACGGGGTAACTCTGTA
GAGTTCGGCGTTAACTGGATGTTGACCCGTATGGAGAATGGTGATCTGAAGGTGTTTAAT
ACGTGTACGAACTTCCTAAAAGAAATGAAAATGTACCACCGAAAGGACGGAAAGATTATC
GACAGAAACGACGATATGATCTCCGCTACCCGGTATGCCCTGTTGATGGCTTCCCGGCAT
GCACGTCCTGGTGCTGTACGAAACAGTGGATACTACAGGAGTGATACTGCAAAGTTGACC
CCTGATTGGTTTGGGAGTATTGTCTAATGGCTAAGCGTCGTCGCAAGATTAAGCCTATGG
ATGATGAACAGGTACTTCGTCATCTAGACCAACTTGTTAACGACGCCCTTGATTTCAACT
CTTCGGAACTTTCAAGCAGCGTTCTGAGGCCCTGAAGTATTACTTCGGAGAGCCCTTCGG
TAACGAGCGCCCTGGGAAGTCCGCGATTGTATCTAGGGACGTTCAAGAGACTGTAGACTG
GATTATGCCTTCTTATGAAGGTATTCACGTCAGGCGGTCAAGTAGTTAAGTATGAACCTC
AGACTGCCGAAGATGTTGAACAGGCAGAGCAAGAGACTGAGTATGTGAACTACCTCTTCA
TGCGTAAGAACGAGGGGTTCAAGGTAATGTTCGACTGGTTCCAAGACACTCTGATGATGA
AGACCGGTGTTGTAAAGGTCTATGTAGAAGAGGTCCTGAACCCTACCTTCGAACGATTCT
CTGGTCTCTGAGGAAATGGTAGCGGATATCCTGGCTGATCCAGACACTGAGATTCTAGCA
CAGAGTGTGGACGAGGATGGAACCTACAGTATTAAAATTCGCAAGGACAAGAAGAAGCGA
GAGATTAAAGTCACCTGTATCAAGCCTGAGAACTTCCTGGTTGATCGGTTGGCTACCTGC
ATTGATGATGCACGCTTCCTCTGTCACCGTGAGAAGTATACCGTAAGTGACCTGAGGCTC
TTGGGTGTTCCCGAGGATGTACTAGATGAGCTTCCATACGATGAGTATGAATTCTCTGAT
AGTCAGCCAGAAAGGTTGGTACGTGATAACTTCGATATGACTGGCCAACTCCAGTACAAC
TCTGGGGATGATGCTGAAGCCAACCGTGAGGTATGGGCCTCTGAGTGCTACACCCTTCTG
GACGTAGATGGGGATGGTATCTCTGAGTTGCGCCGTATCCTGTATGTGGGCGACTACATC
ATCAGCAACGAGCCTTGGGATTGCCGTCCCTTCGCTGACCTGAATGCCTATCGAATTGCC
CATAAGTTCCACGGTATGAGTGTCTACGATAAGATCCGAGACATTCAAGAGATCCGTTCG
GTGCTCATGCGCAACATCATGGATAATATCTACCGGACTAACCAAGGGCGCTCTGTAGTC
CTGATGGTCAGGTTAACCTCGAAGACTTGCTGACCAATGAGGCAGCGGGTATCGTTCGGG
TTAAGGCCATGAACTCTATCATGCCTCTAGAGACTCCTCAGCTGTCTGGGGAAGTCTACG
GTATGCTAGATAGGCTAGAGGCGGATAGGGGTAAGCGGACAGGCATCACTGACCGTACCC
GAGGTCTAGATCAAAACACCCTGCACTCTAACCAAGCGGCTATGAGTGTAAACCAGTTGA
TGACTGCTGCTGAGCAACAGATTGACCTGATTGCCCGGATGTTTGCAGAGACTGGTGTTA
AGCGTTTGTTCCAACTTCTGCATGACCATGCCATCAAGTACCAGAATCAGGAAGAGGTCT
TCCAGCTACGAGGCAAGTGGGTTGCTATTAACCCTGCCAACTGGCGAGAGAGATCCGACC
TGACGGTTACCGTTGGTATCGGCAACATGAACAAAGACCAGCAGATGCTCCACCTTATGC
GTATCTGGGAAATGGCCCAGGCTGTAGTAGGGTGGTGGTCTTGGGGTCCTAGTCTCTGAG
CAGAACCTTTACAATATCCTTAAGGAAGTCACAGAGAACGCTGGGTATAAAGACCCGGAT
CGCTTCTGGACTAACCCGGATTCTCCTGAAGCTCAACAGGCTAAGGCCATCAGGGAGCAG
AAGGAAGCACAACCTAAGCCTGAAGACATCAAAGCCCAAGCTGATGCCCAACGTGCCCAA
TCGGACGCCTTGGCTAAGCAAGCGGAGGCCCAGATGAAGCAGGTAGAGGCTCAGATCCGA
CTGGTTGAGATCGAACTGAAGAAGCAAGAGGCTGTCCTTCAGCAGAGAGAAATGGCCCTT
AAGGAAGCGGAACTACAACTTGAGCGTGATCGTTTCACTTGGGAACGGGCTCGTAACGAA
GCGGAGTATCATCTTGAGGCAACTCAGGCTCGGGCTGCTTATATCGGAGACGGTAAGGTA
CCTGAGACTAAGAAGCCATCTAAGGCAGTGAGGAAATAATCTTGGGAGAACGTTTTGGCT
ATAGTCTAGTGCTGACTGATGCGGCACAACAGATGGTACGTGAGAACGTTCTTCCTGAGC
TTTTCCGTATTGTCCAAGAAGAGATCGAGGGAGAGTGGAAGTCCACGGCACCTAAGGATT
CAGAATCCAGGAGGCCATCTACCACGAACTACATGCCCTCAACCGCAAAGCCAACTTCGT
ATTCAGGTCCTGGATCTGATCACTCTAAAGGAGTATAAGTAATGGACCTTGAGAATCAAG
GCATTGATGGCTCTGAGGCAGCGGAAGCTCGAGATTTGCTAGGTGATGACCTCCTGGCCC
CGGTGATGAAACCGTGATGGTAAAGTCAGGGGAAGAAGAGGAGGAAGAGGTAGTAGACGA
AGGTACTCCAGAGGACACCGAAGAGGTTGAATCTGAGGAGGAAGAAGAGGGTACTGAGGA
AGAACCAGAGGTTGAGTCCGAGGAAGATCCTGAGGATAAACTCTTCGAGATCCCGATTGG
GGATGAAGTCTACGAGGTTAACTTCGAGGAACTCAAAAGTGGCTACCTTCGGAATGAAGA
GTTTGTCACCCGACAGGCTGAACTGGAAGACCAGTATGCAGCTAAGTTCGAGGAGCTTGA
TGCAGAGCGCTCTAATCTACTGGCCGAGCTTGAGCAGTATGCTGTTACCGCTATCGCTGG
TGCTAATCAGTATGACAATGTAAACTGGGAGCAGCTTAAGGCCCAGGATCCAGAGAAGTA
TCAAACCCTTCGACTGGAAGCCCTGGAAGCCCGAGACCGAGCCCAGGTTTCATCAAACGT
CGTAACGACATTAAGGCTATGCAAGAGAAGCGAGCCGAGATTGTCCACTCTGCCTACGTT
AAGCGTCAGACAGAGCTAGCCAAGAAACTCATCCCTGAAATGACTACGGATGAATCTTGG
GGTGACAAGATTGTGTCTTATGGCAAATCGATTGATACTCCGAGGATGAGATCCGAGGGA
TATCTGATGCCCGCCAACTGGCTGTACTCGATGCAGCACGTAAGTGGGCTGAATCTCAGG
TCCGTCGCAAGGCTGCCCTAGAGAAGAAGGAGACTGAGCTACCTGCCGCAGTTAAGCCTG
CTGCTCGTAGGGCAAGCCTCAGAAGGGGTTCCTAAGCGGGTTAAAGCTGCCCGCGCTACT
GAAAGGAAAGACCAGTCGGTTGAAGCCGCTGCTGCTCTCTTCTCCTCTCTCGATATTTTC
TAATAGAAGGAAACGGTAACTTATGGCAACTCAACTAATGCCGTCTCGACTGTAGAGTCA
ACGGTAGCGCGAAGACCTTATCGATATCATCACAACATCGCGCCTATGACACCCCGTTCG
CGGTTGCGCTATCGGCAAGGGCGTAGCTACCTTATCACCACGAGGTGGCGAAGCTGATGA
ACTCCGCCAGCCGGTAGAACTTACCCGAGTGGAAGGTGAGGATGCAACTTCATCAAAGGC
TGGTAGCTTCACCACCATGCTGAACAACTTCTGCCAAATCTCCGGACGAGACCCTGCAAG
TCACCGGTACCGCAGATAAGGTCAAGAAGGCTGGTCGTAAGAACGAACTGGCGTATCAGC
TGGCTAAGAAGTCGAAGGAACTGAAGCTGGACATGGAATACGCCATGGTCGGTGCCCCTC
AGGCTAAGATCCAGCGTAACACCACTACTCCGGGTCAGATGGCAAACATCTTTGCCTACT
ACAAGACAAACGGTTCGGTAGGTGCTAACGGTACCCTGCCAACTGGTGATGGTTCCGACA
CTGGTACCGCTGGTGACCTCCGTCTTCTGACCGAAGACATGCTCCTGAATGCCTCTGAGG
CTATCTGGCGCAACGGTGGTCAGGCTAACTCGATCCAGACTTCGAGCTCGATCAAGAAGG
CCATCAGCAAGAACATGAAGGGTCGTGCAACTGAGATCACCCTGGATGCCTCGGATAACC
GCATTGCGCAGACCGTGGATGTCTACGAGAGTGACTTCGGTAAGTACACCATTCGTGCCA
ACCGCTGGTTCCACGAAAACACCATGTTCATCTTTGATCCGAAGATGCACGCCCTCTGCT
ACCTGCGTCCGTTCTTCCAGCACGAACTGGCTAAGACCGGTGACAGCGAGAAGCGCCAAC
TGCTGGTTGAATACACCCTGCGTGTTAACAACGAGAAGTCCGGTGCTCTGATCCGTGACG
TCGTAGCCCAGGTCTAACCCAACGGCCCTGGGGCCCTTTGTTTCCTTAGAGGTACATCAT
GAGTATCCAATCGACTTATGCAATTGACGAAGACCTTGGGCTCTTTATCCGAAAGGATAC
TCAAGATGTTGAGTCAATCCTGTCCGCTAACGCCGAAGAGCGACACTGGTGTCAATAGCA
ACCGCAAGGATAATATGCGCAAGGTAGCAAGTATCCCCTTGGTTGTCGTAGAGGCACTAC
GGAATCGTCCCATGGCTGAAGGTGGTCCTATCGATCTAAACCTGATTGGTTGTGACCCAG
ATCATGCCGCAAGATTTACCCGTTGGCTTAATGACCGAGATAACTATCGGATGCGGACCT
CTGAGGCACGGGTGTAATGAATGGCTACGATCAATAACGTAACTGATCTCGCTATTGCTG
CTATCCAGTGGTCTGACCGTCAGGATCTTACCCAAGAACTTCTGATGTTGTTCATTGGGA
ACACTACTGACCGTCTTAACCGATTGCTACGAGTGAGGGAGAACGAACACTTCGAAACTC
TCATGGCCTTCGGTGGTGGCATTGAGATCCCTGAGCACTTTGTTGCTCTCCGGTCTATCA
CGGGTGATTCTCTTATCGGTGGTCGTACTCTCCAGTACATCACACAAGATATCTTCACCC
ACTATGTTAACTACAACTACCAACCTCAGGGTGTAACCTACTACACTCGGTTGGGTAACT
TCTGGCGAGTATTCCCCGTTGTTCCTGACGGTGCCCCGTTCATTGTGAACTACTGGACTG
TACTCCCAGAGCTAAGCCTCGCTAACCCAACGACTTGGGCTCTGACGAAGTATCCGCAGA
TTTATCTCTATGGTGTCCTTGAGCAGATTTATCTGTACACCATGGATGAAGCTCGGTCCC
AGTTCTGGGGTCAGAAACTCGAACGGGCTGTTATGGAACTCCAGAACGAGGAGAATGCCG
CTGACTTCGCTAGCACTCGACTGGCGATCAAAGACATTGAACGTTAAGGAGCTTCAGATG
CCTATCTTTCTAAAACCCTCGCAATCAACCTTCTGGCTACCCTCTTCCCAACCAAGGTAA
TCGCTAAGTCCATCGTGACTGGTGCTAAGGCTTTGGCGGAGAAGACTAACTCTAAGGTTG
ACGACGAGTTCGTAAAGTCCTGGAAGACAACCTTAAGGAGAAGACGGAGGATGCAACTTC
TAACTGATAACCTGGGCAGCATCGTCAGTGCCCTGGTCGTATCCTCGGTAGTGACGGTAG
GTTCCTCGGTTGTCGGTCAGAATGTTCATCAGGCTCTGCTTGAAAGAAACATCCAGGCCA
CGGAGAAGCTCACCCAAGCGGTGAGCGATCTCCAGATTTCTATGGCAATCTTTGGGGAGC
GGTATGTCACCCGAAAAGAAATGAAAGAAGAGATAAAGGAGGCCAAGAATGGGTCTTGAG
GTCGCAACTTATATTAACCAGTTGGTGCCTACGAACCCTACCGGCTCTGATCTGAAGTCC
TTTGGGGATGACCATCTGCGACTCATTAAGAGTGCTATTAAGAACACCTTCCCCAATATT
TCTCAGGCTGTCACTGTAACGGCAGCCCAGCTAAACGCAGTCGCTGATACAACCCAGTAT
GTTAAGCCTGGGATGATCGTCATGTGGACTGGTGCCCTAGCTCAGATCCCAGCAGGTTGG
AAGCTTTGTAACGGCGTAGGTACTACCTCTAATGGTATCCCGATCCCTAACCTGATTGGG
GCATTCCCCTGGGGTATCGACGGATCTTCCCAGGCTGTAGGTACTAGAGGCGGTAGCGCT
AACATTGTATGGGATGGGTTCACTGAAGGGACTGCCCTTACCCTAGCCCAAATCCCATCA
CACACCCACACTTGGAGAACTAGGGTGCTACTACCCGCATCGGTTCTGCGGGTGACTCTG
GTGCTCTGACGGGAGGCTCTGGTAACGCAGCCAATACCAACCTTGAGACTGGCCCTGCTG
GTCAAAGTCAGACTCACAACCATGCAGTAAAGATCAACATGCCGTTGGGTAATATCCCAC
CGTTCTGTTCTGTGTTCTTCATCATTAAGAACTGAGGTATACAATGGCCCTAGAGAGACA
AGAGGTCAAGAACCCTACGGGGATTGTGACCGATATCGCCCCCGCTGACCTGCCACTGGA
GAAATGGTCCTTCGGGAACAACGTCCGGTTTAAGAACGGCAAGGCTCAGAAGGCTCTGGG
CCATACGCCTATCTTTGATACAGCCCAGGCCCCAATCTTGGATATGTTCCCTTTCATCCG
GAATAACATCCCATACTGGCTCTTGTGCTCAGAAAAGAGGCTTTACCTGGCAGATGGGAC
TACCATCATTGATGTGTCCCCAGGACCTTACGCTGCTAGTATAACTAATAGGTGGTCTGT
AGGATCGTTCAATGGAGTTATCTTTGCTAACGATGGGGTTAACCCTCCCACCACCTACCA
CCAACTGAGAGTGTGTTTAGGGTTCTACCTAACTTCCCAGCGAATACAACCTTTAGACGT
CTTAAGTCCTTCAAGAACTTCTTGATTGGATTGAATGTAACGTCTAACTCTATTGAAATG
CCCCAGATGGTCTGGTGGAGTACCTCAGCTGATGCTGGTGGTGTCCCGGCCTCTTGGGAC
CCAACGGACCCTACTAAGGATGCTGGTCAGAACACTTTGGCTGATACCAATGGTGCTATC
GTGGATGGTGTGAAGCTTCGTGACTCCTTCATCATCTACAAGGAAGACTCTGTATACTCC
ATGCGGTATATCGGTGGATTGTATATCTTCCAATTCCAGCAATTGTTTAACGACGTTGGT
ATCCTCGGGCCCAACTGTGCAGTAGAGTTCGATGGTAACCATTTCGTTGTAGGCCATGGT
GACGTCTATGTCCACAACGGTGTACAGAAACAGTCCGTGATTGATGCTCAGGTCCGTAAG
TTCTTCTTCTCTGATATCAACCCGGATAACTACCAACGAACCTTTGTGCTAGCTGACCAT
GTGAATACTGAAATGTGGGTATGCTACTCCTCGACTCGATCTGAGCCTGGGAAGCATTGC
GATAGGGCTATCATTTGGAACTGGAAGGAGAACACTTGGAGTATCCGAGACCTCCCTAAC
GTCCTCAGTGGAGCCTATGGCATCATTGACCCTAAGACTTCTAACCTCTGGGATGACGAC
TCCAACCCTGGGATACCGATACGTCGGTCTGGGGTGAAGGTTCGTATAACCCAGCGAAGT
CTAGTATGATCTTCACGTCCTTCCAGGATGCTAAACTCTTCCTCTTCGGGGAGACTTCTA
CGTTCTCTGGACAATCATTCACTAGTACCCTTGAGAGATCCGATATCTATCTCGGGGATG
ATCGAATGATGAAGACAGTGAAGTGCCATCATTCCACACATTACTGGTAACGGTACTTGC
TTAACATCTGGGTGGGTAACGCTCAGGTACAAGGTTCTGGTATTCGATGGAAAGGCCCTT
ACCCATACCGGATTGGTCAGGACTATAAGATTGACACTAAACATGTGGGCCGGTACATAG
CCTTGAAGTTCGACTTCTCTTCGGAGGGATTGGTACTTCAATGGATACACTATTGAAATG
GCCCCGAAGGCAGGTATGCGATGAGTCAAAAGTACAGCCCTTCGATTCCACCACAGGAAG
AGGAGGAACTGCTGCCCTTCCTTAATGAAGAATTCGTTAGGGTTGGACAGACTCTTAATG
ACCTAGCTGACGGCTACTGGGGAACTCTATGGAGCCCCTAAAATTCAAGCCAGGGACAGT
AAAGTATTTTGCCCCAGGTGGTAGGTCAGTATCCGGCATCTATCATTACGACTTGGATAA
CCAATGGAGACTCGCAGGTACTAAACCCAAGGACCTGCCGGGGATTTTATCCTATTTACC
CCACAGAACAACCATCAGCCGATGGGTACCTGTGCCTATAGGATGAACACTGCTAAGGAT
GAAGTATGGATAACCATGCTCATGTCTGGTGGTAACTACACTAATGGAGCTACTGTCCTG
GATCTTCCCCAGGCGTATTGGCCTCCTGCTGAGTTGTTTATCCCGGCCTATTCCAGCATC
ATCCCAGCGCAGAGCACTATTACTTATCCACCTCCGTCTGACCCTAACGCACCTCCGTTG
GATCAAGTCTTTGATGTTCTCAACAGGGCTACTATTCAGACTGGGGTGGTTAACCAAGCA
ATGTTTAAGATCACAGCTAATGGCCGTGTTCTAATCCAAGGAATTCCCCAAGGAGCAGTC
TTCGGGGTACATTCACATTCCCCTGGTGGTGACTCCATGATTCATTTGATTACTCGTGAG
AATATCGATCTGCTTCCTACCGTAGTCCCTGCTCTGGCCCGAGCCTTCAACAGGACGGAC
CTCGGTAAGTTTTGTAGACTTCGAACACTTGGTTCAATTCCTGGTTAATTATGAGGCCTA
TGTCTTCTACCAGGAAGAGAGCGGCTACGCTGGTGTAATTCAAGTGTCCCAAGCACCCTG
GGTAACATCCTTCACTTCTTCTGGAGTGGTAAGATGCCTGGGAATGAAACCCCGGTAATT
ACTCGGAGGTAGACGACTTCCTCGACAGTTCGCCCAACGAGTTAATCCGTCGGTTTATCC
AATGCGAAGGTCGTCGGGGCTGAAGCCTACCCAGAGAAACTCGGGTACACCGAAGACTCC
GTATCCTTCTATCGTGAGGTAACTCCAGATGAACTTCCTCCAATTTAATGTAAAGCGTCT
GATGGGGTTCGATGTAGGGGATGCCTAGATTCTCCGGGCTTACAAGGGTGGTAGTAAACA
AGACTCTGTCTCTACACCTATCAGCAAGGTAACTATAATGAACTACTCTCGGGTGCCAGT
GATTGGCTACATAGCAGGGCTTTGACTAATTACGAGAGCGATCGAACTTTGACCAGTGGC
TGACCAAAATGCTCTCCAACTTGAGTATCAGCGAAGGGGCACAGGTGGTCTCGGGGTGCC
CTTCAGTCTCTCCTGGGTAGCTCAGGTGTTAGCTCTCTGGCTGATTACCTTGGTCCTTAC
GATCCCAACAAAACTGGCTTGGCGAACGCCATTGGTGCAGCCAACGAGCAGATGCAATGG
GACTTCGACACTACGGTACGTCCAGACCTGCGAGCAGGGGCCACTAATGCTGGTCAGTAT
GGGAGTTCCCGAGCGGGTGTAGCAGAAGGTATTGCTACCGCTAGATTGCTCCAGAATCAA
CAGAATAACGCCTCCCAGTTGGCTTTCCAAGACCAGCAGGCTTACAACCAGAATAGGTTG
AATACCCTGGGTAACCTGTCGGCTATTGCTAAGGGCCTCGGCTCTGGTAATGCCATGCAG
GTAGATGCTGGTAGCATGCTTCAGAACCAGGAGCAGCAGGAAATCAACGGGGCTCTACAG
AAGTGGGCCTATGAGAATAACGTCAGCCTTAACGACCTACTCGCCTACAAGGAGCTTATC
TCCGGCGACATGGGTGGAACCAATGTTACTACCTCCTCGGGTGGTGGCGGTGGTGGTGGC
CTCAGGTCTGCCCTGGGTGCTCTCGGTGGTGCTTCCCTGGGTGCTCTCTTCGGTGGCCCA
GCGGGTGCTTCTGTTGGTATGAATGCTGGTGGTAGGGTCGGGGGCCTTCTGTTCTAATGG
CCTCCCTTCCTCAGAAACTGTTCGCTATAGGACAGAACATAGGTGGTGGCAGGAGCGGGT
TCAGTTGAGTCGTTGGGGGCTCCTACCGCCCAACCCACCTTGGGACAATGCAATCGGGAG
AGTGGACAATCAAACCCTTTTGGCGCAATGGGCGGAGCAGCACTCGCGGCTCTCCTCGGG
AAAGAGTGGAAGTCTTCTTCAGAAGCAGTACCAAGCTTTTTCTGTCGAAGGGGCTCGGGG
AGCAAGCGAGCGAGGCGCAGCGGAAGTTGCAGCAAGTATGGGGCAGGTGGAATCGCTTCT
AGCGCAAGAGAACTCGGTTTCGGACAGCAGCCAAAGTCTGGAATCCTAAGTAAACTATTT
GGAGGTTAATCCTATGGCCAGCATGGCTTATGAAGGGTCCCCAATTCGCCCTAGCATCCT
CCGGGCTGCTCAGAATGAACTGGACATGGCGCGAATCGCAAGGAACAAACTGCCGCTGGC
TGTAGAGGGCTCAGGAATCCCTGATCGTGTCCGCCGGGCTGCTCTCAGGCTGCCCTAGAG
AACCCTAATCGTTGGTCTCGGGTTGCCAAGAGGTGGCCCCTGCGGCTGAGGCTACGGGTC
GTGGAGCCTTGGGCCGTATCGCAGGTATCCTCGGTGGTCCAGAAGTGTCTGTTGGTGCCC
AACTGGCTGTAAGCCCAGGAGAACTTTGGGATGCAGAGCGGACTCGTGCTGAAGAAATGG
CTCAGGCTAGTCAGGTTGTAGAGAACAAGGGTCCCGGGAGGTTGCTCAAGAGGCAAACCA
CTGGCCTATAGGGTAGGGCCAACGTGCAGCCCAGAACGCCACAGGGGTTCTACGGGGCTG
AACTCCTGTCCCATGGGGTAAATCCCTAACCAACCCCTCTATCGGTTCGAGATCACTCCA
GAGGTGGCTCTGAGGCTGGTGCTGCTGTAGCAGATGAGGAGGAGGCTAATCGTCAGGTTA
TCCAACAGGGTGCAGCTGAGGGTCTTCGTACTGGTGCTGTAAGTCGTCCTGAAATGGCCC
AAGCTGTAGTTGAGGCTGATGCCCAGCGAGAAGGTGTAGAGCTTAAACCCCAAGAGCTTA
AGAACCGGGTGAACGAAGAGTTGACCCAGATGAGGACTATGGACAATGATGACCTGTCTC
GTTATGTATCTTATGCTCTCATTGGTACTGGTCTTCTGGCTTCGGCCCTAGACAAGACTG
GTAAAGCTGGGGATATGTTCGCTGCTTCCTACGAGCGTCAGCTTGATCGTAATCTACAGG
CTGGGATCAACCAGCAGAAGATGGCTGCCGCAGCTGCTGATCGTCAGATCAAGGAAAGGA
TCTGGAGCGTAAGGTGGCTAAGGATGCTGCTGATGTACGTCTCGGGGAAGGAAACCTGGA
GGTCAAGAAGGGCACCCTTGAGAAACCTCTCGTAGAAACACTGGTCTTCTGGATCGGTGG
GCTGAGGAGGCTGCCTCGGGGAGAGCCAACCTTGCCCTAACCCAGCGTGGACAGGATCTG
GCGCTAATCAGCAGGCCAACTACGGGCTGAAACAACCCGTCGTGGCCAGGATATGTCTCA
AGAGAATCCAACGAAAGTTCTGCTGTACGTCTGAAGACTGCTAAGATCAGCGCTCAGGCA
CGGCGGCAGCTAAGAGCAGCCGAGGTGAAACTGTTACTACTAGGACGCCCTTGGTATCCT
GAGTGAAGTCAATGGGTTCTCAGGCTCTGGGTGGTAAGAAGCTGAGTAAGACTGCCCAAC
AGGCTATTGCCACTGTCGAAATGAACTTCGGGCTAATCCGGGGGGGGCTAATATTACTTG
GGTGTTATTATCCAGCGGGAGGCTGCTAAGCTTCAACCTACGGGAACTGGTTTGGTGCTG
ATCTGGACTATCCTGCTAACCCGATAAAGGTACTTGACAAAGTATCCAAAATGTTTATAA
TGAACCTAGGCTCTTAAGATTCGCTTCTTCCTTTTGACTTGCCTTACTAAGAGGTTAGTA
TCCTAAGATACATCTTAAGAGCCTATTCTCTTCCACAAATAAAAAGGTGACTATGGCTCT
TTCTCCAGAACTGAAAGCCGCTATTGATGCGGAACTGGCTGAGGTCAAGGGCCTCGACGT
AGCCGCTGAATTGGCTTCCTTGAATGAGCCAGAGGTTATCGAAGAGGCACCCCAGGAAGT
AGCATGCCTGAGCCTACCCCAGACCTGAGTGCCCCTAGTAACTCCGGCTGACCCTAATTC
TATTAGCTCTGCGATTGGTCGCGGGGTTGATACCATGCAGTCTAACATCGGTGGTACCAT
TGCTACCCTTGGTGAACTGACTGGCAGTGACTACCTGAAAGACTACGGCACACAGATGGC
CGAGGAGAATGCTCAGGAAGCGTCCCAGTACGGAACCCCTAATGTTCGGTCTTTCGCTGA
TATCCAAGATATCCCATCCTTCGGTTCGTTCCTAAAGAATAACATTGTAGAGGCTCTCCC
TTCGATGGCCCCTGTACTTGCTGGTGGTGCCGCAGGTGCTAAGGCTGGCTCTGTCTTTGG
GGCCCCAGGTCGTATGGGTGGAGCGCTCATTGGTTCCTTCCTGAGTTCCATGGGTATCAA
CGTAGGTGCCCTGAGTAACCAGATGAAGGAACTAGACCCCGATCAAAGCAACCCATGGAC
CGCAGTCCTAGGTGGTGCTGGTCTGTCTGCCCTGGATACCGCAGGGGCTGGGGTTATCGC
TGGTCCCCTGTTGAAGCACCTTGGGAAAGACGGGGGGGCGCCACCATATGCTGGTACAGT
CCGGTCTACCCAAACAAACCGCTATCGAGGCTGTTACCCAGGCTGGTAAACACGCTGTAG
TCTCTGGTGTAGCTGAAGGTGTTACCTCTGGTGCCCAGCAGGCCCTCCAGGACACTATCG
CGTATGATGCCGTTGGGCAGACCCAATCCCTGAGCAGTTCATGGATAACCTCCTGACTGC
TGCATTCACTGGTACCGCTATGGGTACTGCCGGTGGTGCTGTATCCTCGGGGATGGACAC
CCTGGGTCGTCATCAGGACTCCGCTGGATCTGCCGTGGTTGACCCTAACGCCCCTGCGGC
CTCCTAAGGCTTCTGAGTTTCAGGCCTGGGGGTACGATCAAGAAGGCTTAAGGATGCCCT
GGAAACGAAGCTACGTCCCCTCCCTAGAGCCACTCGCCAAGGCCTCCCCTATCGCTCGTG
AGTTCTCTGAGACCTTCCGTGCTGATATGAGTGGTAAGCGGGCCTCTGGTAAGACTATCT
TTGAGGACCAGGAACTACAGGCAGGTAAGTGGAACTCTGAACTAGATAACATCTTTGAGG
GTAAGTCTTCCAAAGAGATCGATAGGATTATCGCTGATACATCCGCTGGTGTCAACACCC
TGAGGCTACCCGTCTGCGTTCTTTGATGGATGACGTAAGGAATGAGGCTGTTAACCGTGG
TGGCATGTCTGTTGGGACCATCCCCAACTATATGCCCTTTGGGCTGTCTCCTGAGAAGGT
TCAATCCCCAGAGTTCCTGAACGACATTACTCCGTACTTCCAGAACCGACAAGCTGCTGA
AGATGCTGTAGCTAACTGGCTGGTTGAAGTCTCGGATGATACTCGTGGTAACACTGCCCT
GAGGTTAACCGATTGGTTACTCAGAACCAGCAGACCGGGGCTTGGGAAGTAGACCCTCGC
TACCGTATCCAGGGTGATCCAGATACTCTACGTGGACGGTTTGCCCAGAGTGATGCAGTA
CCTAAGTATGGTCAACTGGAAGAGAGTCGGGCCTTTGGATCTGTACCTCAGGAGATACTC
AATAAGTATTCTCTTAATGATACTCCTAAGCGTCTCCAAGAGATTCGGGACTACTTCGAG
GGCTTCCACCGGATTGCCTTTACCGAACGGTTTGGTATCAACGGGGAGAAGGCTAACGCT
AAGATTGCATCTGCTGTAGCTGAGGCTCAACGGGCTGGTAAGCGGGTTACCAAGGAAGAG
GTGGATCGGATGTATGACCTAGTGGATGCCTATAATGGTATGCACGGTCGTATCAAAGAT
CTAATATCAAGCTGGCGGCTGTTACGTCGAGATTGCCCTCGTGCTCTCCAGGCTTCCCTC
GCCGGCTTCTCGACCTAACCGAGTTCAGCCTACCATTCGCTAAGGCTGGGGTTATGCCTA
CCCTTGGGGCTGTACTCCCAACCATGGGTGAGGTAGTAAGGCAAGCAGCCCGCAGGATCT
ACAGTGGGATTCCGAAGTCTGAGACCGGTCGGTTTTATGAGTGATATGAACCACACCCTG
GCCTCTGCTACCTCTCTGATGGCTGATCGGGTTGGTGCTGAGGTGTTTAACTCTACTATC
AAAGACGATCCGTGGTCAGTTCCTCATTAACGGCTTGTCCATCCTAACCCATGTTAACCG
AGTGTTTGCAACAGAGACTGCCAAGCGGGTCTATCAGAACAACCTGATGGATCTAGCCGC
AGGGCTCCCCTTCAGTTCTGCCAATGGTGCTCTAAAGGTTGCTCAGCTTCGGGAAATGGG
GGTTAACATCAGTAACCAGCAGGATATCTCAAGCTAATCTCCCCAGCTACTCCATCTGAA
GTCCTGATGGCTAACAACGTCAAGACTTTGGCCATGCGTCGGTTTGTAGATCAGGTAGTC
CTCGACCCCACCTTCGCTGATAAGCCTATGTGGATGAGCAACGGAAACGTTCAGATGTTC
TCCCTCTTGAAGGGTTACCCAGCAGCCTATGGTAACATCATCCTACCGATGTTCCGTAGA
CGAATGAGCCCACACTTCGCTGGTTCCTGACCAACGCTGGTATGGGTGCCGCAGGTGTAG
CCTTCACTCTGGTCTGATGATGAGTTCCATCCAGGTATCTCCAGATGAAAGCTTCGTCAA
TCATTAAGTTCGGTGGTCTAGCCGAGAGATACCGCAGTCCGAACAACGGATGATGGACGG
TGATGCAACAGATGCCACCCAGGCTTCGCGATTTACGACATGCTGACGGGTTACTGTCGA
GGGACTACCCCCGCAGAGGTAGTCTTGGGTCCAGTAGCTGGGGCCGCAAACTGAGGGTGC
TATGGCTGTTGGTAAAACTATCGCTTCGTTTGGGGACGACCCATCCGCAGGTGAAATCTG
GAAGTTTCTTGTATAAGAACTCCAGCACGTCCTTTCTGTTTCCGGTATGGAAGCGATGGA
AGATGCTCTGGATCTTTATGAGAAGTAATATGGCAACTTATAGAAACTGATGCCGCTGGT
CCTGGTGGCCGTGCCTACGGTTGATGTGCTAAAGTCTTCCGACGTAATGCCGATGGGGAT
CTCATTGAGATTACCTCGGATGCCGGTCGGGTGAATACTTCAACATCGTTACCGCTACAG
AGTTCCTCCCTGACTATTATGCAGTTAAGGAGAACGCAGCCCTGGTAGTAGAGAACGTTA
CAGCCTCCTTCCCGGAGAACCCCTCAGAGGCCGTTATCCCCTCTGTCCTGGTCAATACCC
TCGTGGGTGCTCCTGGGGAAACCTCGGGTGGCAACCGTGACCCTGGCCACGTGATTGGTC
TGTTCTCTGAGACCATGATTGAGGCACCTTGCGGTACTGCATTCGGTAGTGAATTCCGAG
TAGACCCTCGTCGGGAACACCTGGGTGTTTACGTGGCTATTAAGCACGTGATTGGCCCGG
ATGACCAAAACGGTGGTACCATCGGGGATTACATTATCGAGCAGTTTGATGACATGCGTG
GTCCTGTGAAGAACATTGGTTCTCTCCAACAGAACTACCTGGATCCCCGCCTTGTGACTA
CTCACCTTGGTGGTAATGTCGTCAACACCCAGCAGCTTACTCAGAACATTACCCTGACTA
AGCAGCAGTCTGGTTGGTTCTTCATGTCTCTAGGGACCACGGATATCACCGTGACCCTTG
GCGGATGTAACCCAGGTTGTCACTTCCACTTTATCCAGGGATCGGCAGCTAAGATCAAGT
TTGCTGTATCTCCGGATAAGGCTTGGTACGCTAAGGGTAACCAGACGGAAACTGATGGTC
AATTCGGGAATGCACTGTCCGTGTATATCCCTTCGGTGGTACCGTGGGTACCTTTAAATC
GGCAGCAATTAGGAGTTAGGTTATGGCAGGTTATATCGCTGACAACGACGGCAATAGTGT
ATTCGTTATTCAATCGAAGGCTATTGCTGTACGTAATGAAGATGGGACCTTTCGACAAGT
CATTGGCCCGAAAGACTTTGAGCCAGTGAAATGGGAGGATATTGAGGGAAGCCTGAGCTT
CTGGAGGTGGGTGCTAAGGCTAGCGAGGCTAAACCAGGTAACTGGAAGCCCAAGGCTAGC
GAAGTGAGTGGCCTAGATGCAGCCGCGGCTTACGTCCAGGGTAAAGGTCGCTGAGATCCC
GTGGCTTCCCTATACGGACACTCACGAGGCCCTCGTGGATAAGTTCAACACACTGCTGAA
CGCCTCTCGGAGCTAAAGATAAGGCCCCTTGGGATGATACCCTTGGGGCCTTTTTTTTTC
GAGACCTCGATGGATACCTACAGTGAACCTGCCCAAGATAATACATCGATGAGGAGTCTG
GATATTTCTCCCAGGGTCAGGAGTAACATCAGTCAGCTATATCCTACATAAACCAGCAGG
GCAAACAAGGCTGCTACCGCAATGTAGGTCCAAAGGGAATCCACCCTTACCCTTTTGATC
TTCGCGCATAGCCTCCAGTCTTTATAGAAGACGTTTGACCGTTGATCTACGATATCCAGC
ATCGAGCTAAGGAAGAGATATTCTTCCGTAATCTCTCGGACTTTATCAGAAGCAAGCTCC
AGTTCCGCTTGGACCTTCGCAAGATTGCTCTCCGCCTCTTCAAGTGCCTTCTCCAGTTCA
GCCTTTGTCATAGTACACCACCTTGTCACTACCCTTACGACATACTCAGGTCAATGGTCC
AACTCATGGTCGGGCACTTGATCCGAATCATCTTGGGGAGCCATCAACTGTTCCAATAGC
TAGGTGGGAAGAGGTCTTCTGTGTACCAGAAAGACTACCTTGTCTTCTCCCACCTCTAAG
TTCACGATCCTACACGATGCTTTCATAGTTACCCAAGATATCGGAAGGTGAACACTGGGG
ATGTACAGGATCTCCAGGACCTCGATGGAATACCATCCACCTTAAGCCCCTCCTCTGCAC
CTTTCCCAAGCTTCATATCAGCCAGAGTTCTGCGGAGATCGGCGTCTTAAACATCAACCC
GTTGACCGAAGTCATACTCAGACTTAATGTAGTACCAGGTCTAAGAGCTTTTCACAGGAA
TTCTCCACACTCATCAGCTTGAGGTCATACTCTCCGTCCTTGTGGTTCCGGAGAAGGGCT
ACCACCTCGCCAGTGGTCGTTACCCTGGGGCCCTTTGTAGGACTCATCATGGGTGTGCCA
AAGCTACCGACTACAAGCCCGTGGATTCGCTTCTTGCCCTTCTGCTGCCTCGATGTGGTG
CTTGGATCCTGTTCATTGGCCTTGGACGGGAAGCTGCGCTTGCGTCTTATTGAGGGCGTG
TAAACTCCGCCGTATGGCCGACCACTGTTGGGATTGTAGAAGTAGTTAGTGGGCCGGCGA
CACCTTCGACTTACCACGTCGAGGAAATCATGGACAATCCAATCGGACAATCAAAATGAT
CGTGCCCAATAAGGCAACCCAGGCAGGGTTGATTTTTCATGTGCAAGCGATACGCTCTTC
GTGGTTTCCGATGAAGAAGTGATTTCTGGTCGGTGAATGCGCTTCTTACAACCCGCTTGG
TGTTGCTGTAGGCGGCGGCGAAGAGGATCGAGGCAGAACTCGCATCGCATCATTACCAGC
TTGTATGTCAGCGAGGACTCGACGGCCTTCGATCTTAGCGGTACCACGGTCGTAGCTTGA
CAGACTGGGCATGTCCCAATGGTCCCCAATATGAACAATTCGCTTGGGGCGGTTACGTGC
GATCCACTCCCCAAGGTTCCGAGGTGATCGATGTTGACCTCGGATCGAGCAGATTTCAAT
GGCGAGTGTCTTCATTATACTTCTTACTCATTAGGGAAGGACTCCTTGAAAGTCTCTAGA
GTAAACCAGCGGAAGCCGTTACGCTCGGCCCATTCCGCCATGGATTGCTTTGTGCCATCT
TTACGCTTGGAAAGCCAATGGCAAGCTTGTGTTAGGGCGCTCGTAAACACAAGTTCAGTA
TCTGGGGGAGAGCTTCCCTCACCCAGATATTGCCAGATGCCTCTGAAGCTTCCTGGAGAA
TTGCCCTTGACCTCACGACCAACAGGTTCTTCGTCGATCCACAGGGTTGTATTGTCGATA
CCTTGAAGTCCGGCGTAGCGATGACGTATGGCACCGGCTCCGGCGAACTGCATCCAAGGC
ATCGTCTCGTGGACGATCCTCTCGGTTTCGCTGTCGTACTTCGTCCCAATCAATGACTTC
AGTTTGGGTCTGGGCCTCCTGCTTTGCCAATGAGCCCTCCTTCTTGAAGCTTACGGACAA
CCGATAGCGTCCCCTCAGTGAGGACAGAGTTAGACCGGATCAGCAATGAAGCCAATCCCC
AGGCACACCCGGAACTCCGGATACTTAAGTTTGATACGTTGGATGAGACAACGCGCACCC
AACTTGATTGTCAGTTAGGGGTTGAGAATGATCCAGAAGGGGTCTAGGACTCGGTTAAGA
GTAATGCACTTCCTTGCTTTATCAAACTCATAACCCTTGAACGGTAGACTTGAGATTTCA
GTCCACGGACACTTATGGGCCAGCATGCAATCTCACACAGTCCTACATCGAACTCTGCTG
GATGGGAGTGGGTACTCACGACAAGGTTGACGGGTAGGTCAACACCAATGGATTCCCACA
CATCAAACCCCGCAGGATACCCATCAGGCTTATCCCCAGTCCTACACCGGACCTTCTTGT
GGCCCAGCTTGTTGTAGAATCGAGTGACCTCTACCTCTAGGGTATCGTCATCACAGGGGA
TCAACCGCGGTCCAAAGCGACTACGCCGAGTATCAGTGGGGTCCTCCACATGGCCTACGG
ACATAGAGGTCGATATCCTTAGGTGCAACCCGTTGTCTGCATCGAAGGAAATACCAGCGA
CCTTGTAGTCGAACCCAAGACCTTCGAGGGATCTCAAAGAGACCTTCCACAGATCCTCTG
TGCTTCATCAATAGAATATTTCTTCATCGTCATGAAGTCTCCCAATGTGTTTCTTCAGTG
AGCAGTGATACTCGTTACCGGACTGCTGAATATCAAATAAAGAGCGGTCCCGAATGAGGT
AGGGGTGAAAATTCTCCAGAAGGTATTGCTCTTTCCAATTATCCGTACTGCTTCTGTTGG
TAGGCGTAGCGCAAGCTGCGAAGAGATCTCTCGCTAAGCTCCCCAAGCAAATCTGCCGCT
GTCTTTGGTCCGTCCCTGGGAGGCCTGGTATGTTATCCACGGAGTCACCAACAGGAGTTG
CCCGTAGAGGAATGCCGTACCGTTGCCCTCAAGCTTCCATGATTTCTGCGGCTTCTTAAC
GCCATACATTCTCCAAGAAGCGGAGACCCTTCCAAGACCATCAACTTGAAACGGTCCCAA
CTCCGGCTGGGAATCTCCACAGGGCCAACTGTAATGGTAGCATCCTGGGACTTGGCGGAT
ATCCTTGTCTCTAGAACACGCTGTAAAGTTATCTCCCTCTTCGATCCCTCGAATTCCGAG
CCAGTCATCAACCTCGATACCGTGGAAAGTGATTGCTCCCCACTTGTCATAAGTCTGGCC
GACACGGTTTCCCAGTGATGAGGCTTCTCAAGACCGATTCGATACCCTTATATTGCTTGA
TTTTCGCAAGCTCCAGCCTAAAATTTCCTTGGCCAGTAAGGTAGATTTCACACTCATCTG
CATTTACCCTTTTGATAATTTGTTCGACTTTATCATCGACGAGCTTATGGACCTCTTCCT
CAGGCCATGGCTTCTCCACCTGGATATCGAAGATGTGTTCCTTCGACATAGCCACGTGTC
CCAATTCGTAGCGAAGCTAAAGTACGTCGCCATCAATTCCTGCGATCATACTACTTACCC
TCCTTACGCTGGACTACTCCCAGCATTGAGCCCACGCTCTGCCGTACCTTATGTTCTGAA
CAGCATTATATGGGAACCCTAGTTTAGAGATTTCTGTATTCCCTAGACCACTTTTGATTA
ATCGGATGATCTCTTTGGCAACGCCCTCGTCTATAGTGGCAGCTGGGTTCTTGCTACCTC
TAAAGTCTGCGTTTCTCCCTTTCCTTGATCTATCCGTGTTGTTGTCACCCTTAGTTCCAA
GGAATAGGTGATCTGGTCTTACACAAGATGGGTTATCGCACTCGTGAAGTACATATAGTC
CTTCTGGAATCCCTCGAAGTGCAACTCCCAGGAAAACCTAGGGCGTACCCAACTCGTTTG
TTCCACTTGAAGCGACTTCTACCGAACTTATCAGTAGATGCTGTCCAGTTCCAGCAACCC
TCAGTTTTATCAACCTTGGACCAGAACCTCTCTTCAATCATCACGTTACCTCTTCTACGA
CCTGAGACTCTTTTAGCTTCTCTTCCCAGAAGACACGAAGGGCAAAGGCTTCACCCTTGG
CTCGATACTCTGGGTGCTCTACCACCGGGTTAACCAGTGCCTCGTAGCCCTTCCAGTCGT
AATATTCACTCTCTTGACGCTTGGCCTTGTCCATAGCTTCCTCCGTTAGGCTGTAGCCTG
GGGAAGCCCCAGGTCTATCGACGCCAATCACAGAGTAGTTGCTAGGAACAGTCTTAGCCC
TTGGGTCCAACAGAAGTTCCATTGGATTCCTCCTCTTGCTGAATGACGAGTTCAGGGATG
CTAGGGCGTTCCCACACTTCGTGATACCAGTCAGGGTAGTTTACTTTGGGGGGTCCAAAG
CTTCACCCTTGTGTCGCTCAAGGAGGTGTCTCCCGAAGGCAGCGTCATATCGCTGGACTG
TTATAGAACAGACCTCAACCCCACTGACTATATTTGCTGGCTCCGAAGTTAGCAGCGGTG
CCCACTGCATATAGAGCCCTCGGGAACGCTTCGAAGATGATTCCAACATCCACCTTACCG
CTATCAAGTTTAGCGCCCGGCTCACCGGATTGATTCCATTAGGATCGATACTCTACTTGT
TAAGATTGTATGGAACTCACCGCTGGCGCACTTTACTCTCGATTTCGAATTAAGCAGTAG
GGGCAGCCTGACCGAAAATACCCCGACTCATGATCCGCTAGGCCCTTAGATAGGAGCGTC
TTCGTCATCGAATACCTCGAATACGTCCTCAGGCTCTTCCTCGGTGTTGAAGGCACCACC
GGAACAGCGGATCGTCATCACCGTCGCCACCGGATACGATCTCGTGGAGTCGTACACCGC
TTTTGATCATAGGGCCGTCATAGTACCCGCCTCGTGGGCGCTTTTGGGTCTCGATCAGGA
TGCCAACTTCACCAGTAGAGCCACCCCACGGGTTGGGGCCATCATACGGCTTGTTGTCCG
AGGTCAGAACCACCGGCTTCATTACAGCAGACTTGCGCTCACCATCTTGCATCAATCCAC
GTGCTTCTTCTGCTTGACCTTGAACAGGTACTCGCCATCACGCTCTTGGTATTTGATGGT
ACCCTTCAGACGGGCATCTTTAGTTCCATCTGCTGTACCAGCTTGTCGGCCTCTTCCTTG
GTCGGAATGCAGATGGTTACTTGGTAGACCTCACCGTACTCGGTATCCGGAGTTACGATG
TAGGGAAGATCAGCTTCGCTACCGGGAAGGTGAATCGTTGGGAAACTTTCTTAGACATAT
CTTAAACCTCTCGTAGGGAAGAAGGTATTATACCACAAGGGGCTGAAAGTCAGCCCCCAG
ATGTATCAGTGTGTGTCATACCATGAGCGACCAGCGATAGAATCACTAGCCAAAGGACAG
TTAACCCCGAGGAGTTCCCTCAGCATCCCGAACGCAGACTTCCATCTGCTCACGTAGCGC
CTTTGAGATCCTCTGGGTGGCATTCCCACTGGCCTTCGTCGTGGATATCCAAGACTTTCC
AAGCCCTCAGGTTTAACCTACGAACCCGCTCGTCTAGGAGCACCATGGCCCACTTCATGA
CAATAGCACCTGCTGCTTGTAGGAGGGTGTTCAATGCCTTATGGATCATCACGTCGCCAG
ACTCAGAACGTCGCATGGTTAGCTTTTCGTCCGTCTAGCCCTTCGAGATAACCTCTCTCG
GCTTCCTGCTTAGCCACTCTCAATCAGTGCAGCTTAAGGTCAGGGTTAGCCTCAAGGAAC
TGGGCGGAGTCTAGCCCGTCTGCCCTGGTGCCTCAATGATAGTCCCGATCTTGGCATCCC
CAGCACCGTAGATGAACGCATCAAAGTTCAACCAGTGTCGCTACTACTGGCCCGCCTCTC
GGCTGCCCCAAGGTTTCCCCTGGGGATCGGACTATATCATCACCCTGTCTTCCAGGGGCT
GGGCGCTTCGAACCCGCTTGGGTCCTACATAATAGTCTCTGCACCTTTACACTTGAAGAT
ACTCAGCAGCCTTCCGAAGAAGCTCTGGAGAATCTTTCATCAGTCCCAATACTCTGTTAC
AGTTGTGACACAAGAGTCCACGAACCTTTACTACAGTTGCATGGCAGGGTCAACGACCAA
GAATGCAATGTGCTTAGAGGGGTCCATCAGGAATCCTTCTCCCCGCAGATATCACACTTG
TGGTCTCGTTCATCAGCCATAGCCCTTACTTCATCATACGTGAGTCCATAGACTCTGGTG
TAGTAGGCATTGATCCTACCCTCCTTGCACTCGTCTGAGCAGTAGTGGTGGCTAGGGGGG
GCGTAGGCTGGAACTCCGAGGAGCACTTTCTACAGTTCTTAATCTTAAACCATCCATTTG
GGTACTTCATAAACACCTCCCATAAAATATTAGTATAACACATTCTACGGGAAATGTCAA
GTGTCTTGGCTCAGGATTCCCATATTAAAGGGTTCCCTGAGTTCACCCAGTTTTAATCGA
GGGTTTCCCCAAGTCATCCATATCTCACAGATGAAGGTCTTAGCATCGTCCCGTGTGGGA
AGCCCTGCGGCTATCTGGTTGGCCGTGTGTACATCACCCTCGACCACCTCTTTGGTGTAC
TCTGGGTTGTTGATGTAGGATGCAAGCATCGAAGCTCTAGACCAACACCATCGTAGCCCA
CGAATACCAATTGATTCGCTGGGACGTATGCCAGTACTTTCCTACAGTCTTCCACTTACC
CTTCTTGAGTTCCTCCACGATGTTAGTGAAGGGCCTAACCCTTTCGTTCTCCCCAAGATC
ACGGCGGAGGACAGTCCATTTCCATTCACCAGCCTCAGGTGTCCCCTGGAACAACCCACG
GATTTCAGGTCCGAAAGGAGAACGAGCAGCGGGGATATTGACTACTACGGTGCCTCATGC
GGCCCGTAGGTGTTACTGAGGGGTTAGCACTCCCTCTAGGCGTCCATCAGGCCTCACCTG
ATCTACCATGCAAGGGACCGACGGTGGGCCCAAACCATTCGCTGCATCACCAGCTTTCCA
AGGTCGCCCTGGACGGTATCCATCGACTCTTCCGTCATCTTCGGAGTCGTTGGGACCTTA
CGTTGCTTTCGAGCTTTCTCTTGTTTCCCCAATGGTAGTTTCCCAGGGATGATTCCCTGG
GCTTCGTCGGAGTCGTCCAGATTTGCTCTGTCGAGGATCCCTCAATGTACTTATTCAGAT
TCATTAAGGTCAGATCACGTAGCTTCTTGCCATCAGTGCCGACAGTAATATCCTTGATGT
TCCATTGGTCAGGTATCCATCCCTGATCCAGCATCCAGTCCTTGAACTTGGCAGTCTTAC
CAAGGTCGAAGGGTACATACTGATTGCAGAGAAGGGACCAGATACCTCGAAGTGCCCAAG
ACGTTGCCACAGGGACTGGAGTCTTTGGTTCGGCTTACCACTCTTTAGGAATGGCTTGCT
GAAGGCCCCAGCCCTTACGATCATCATGGGCATCTGGGGACAGCCTCAAGGTCAATCTTG
AGGATTCGTTCGCTAAGCACATGGATCAGCCACTTGGCCCGCTGAGTCTGGAAGTAGACT
CCACGTTTACTCTGGCGGCTGATGATATCAGCTACCTTGTGCTCAACGAACAGTGACTTT
CTCCAATCCACTTAATCCCCTTAAGACGCTCCGCCAGATTCTCTACAACTGTCTCCACAG
CTTCGTGGGATACACCCTCCAGGTAGGCGAGGTAGTCCACGGTACCACTGCGGTCCAGCT
TCCATTGCTGCTCCAGGAAGGCCACAGCAACTTGCTTGAGCAGGAAGTCATCTACCCGCT
CCATGAGGATACTCTTGATCTTGCTCATGTAATCACCAGTCGAAAATGAAGTAGGAACAC
CCACGTTCACAGGCCAGGTCTACCAGGTACCAGGACAGGTCGATCTTGCGCTGGATATCC
TTAATGTCGCTCAGTTCTTCATCGGATGGCAGATTCACTACCCTTCTCCTTCATGGATCA
AAACTCCATTTCTTCTCTTCTTGAGAAGTAAACAAGAAGGTCTCCGCGGATCTAGCTGGG
TATTCCTGTCGATGTAGAATCGTCGTACGAGCTCAGATCCTTCTCCCGTACGGCTTTATT
GCGTTTAAGGCGGTAGGAACTCCAGATCCAGGTGAGACCTGGTAGCTCACCAGGAATCAG
CGGGGCTGTACCCTTCTCCCGAAGAAGGGTAATTTGGGGACTGCTGAGGAACTTCCGGAT
GTTAGTCCTTTGTATCTCCATGCTCTTATGGAGGGCAGAGGCCATATTACACAGGTACTT
ACCACGGACATGTTTGGAATAGCTTACATGGTCACTCATTTGTATCTCCTATAGGGGTAA
AGCAACCCCGGACACCTTAAGGTTATTATACCACACTTTTCTGGATTTGTCAAGCCTTCT
TGTGGAATACTTTATCGTACCACTTGTAGAAGTATTGAGCAGAAGTAATACCAAGGGCAC
CTCCCCACCCGGACACGAAGATAAACCATCCGATATCAAATTCTGGAAGATCCCACGTGA
TGTAAAGCAAACTGAGCCAGCGTGATACACCAAGATACTACAAACCCAGCCTTGATCTTA
TCATCCCGAAGGAGTTTAGAGTTCAGACCCAGGAGAAATACCTGGAAGAATGCACTCAGA
AGATGACTAGGACTTGTAGGGTTATTGACATGACTCACACACCTCGGGGTCTAGGCCGCA
GGCACTACCCAAGGGGAAGTCATCAGCGTCGATCTCAGGGCCACCCAGCTACCAGGAATT
CCTTCTCCAGGGCGATCTCCTTAAGCGCCTGCTCTTCCAGGCTTTCCTTCTTACTGGTCA
TCATTGATCTCCTCTACAGAAACCCATCCACGGACAGCACCGATCTCGTTAGCTCGGACG
GTCATGGGGTTGGCATCGGAATCACCAGCGAAGAATCTGATGATATCCAACTCTATGCGG
TAGAAGTCTGCGTTGAACACCTTGGTGTCCTGGAATCCCACGTTAACTCGATACTTCTTC
ACTCTTACCTCCCAGCAGTTCTTCCAGATTAGCCAGTACCTTCTGGGCCTTCAGATTGTC
CTGCATGACCTGGGTACGCTCCGCCTTCAGAGGAGACTGATCTCGTTGGCCAGATCCGTT
CAGATTCCTCCGAAGACTCTTGGATGAAGGTCTTCAGGTTAACAACTTTTAACGATAGAG
GGCTGAGGATTGCTGATAGCAGCTTTCGATTGAACATAATGAGTCCTCGGGGCCCGAGCC
CCTCTTCATTACTTTTCTTGCGAATGCCTTTGGCCTTACGATCGCCCACCCGATACCATC
GGCGGCGTTGAAGGAACGTACGGACGCGAAGGTTAGAATCCCCGTTTATACACCATCTTT
AACACGTTTGATATGATCGACAACCTCTGGGTTCGAGCTTCCCTGCCAAGACGCTTTCCA
CCTTTCAGGCGGGCTCGGTGCCGCTGAGCATCTCCTGACTTAGAGCCCACGCGCCCGTTT
CGCCGCGTCGAATAGCCAGTTCTCTCACGCTTGTAGTTCCTCACTCGCTTTTTTAGCCAA
TCTACAGTCCTCCATTCAACAAGCTCTTGCCAGAATCATGCCAGTACCCGGAGGAGGTAG
TTGAACAGTCGTGACAGCCACTCCATCCTGAAGGGTATGCTCAAACATTTCCTCACAGAA
CTTGGAGAAGTCGTTGAAGTCACCTTGTGACACTTCAGGAACTTACCCAGGCTCCGGGAA
TGACCCTGGGGCCTATCTGTAGAATCAACCTCGATGCAACCAAGGTATCATAGCTTCGGC
CGGGGGGGGTATGATAATCCCATGAAGTTTCTCAAGGACCCGGTTGTCGTAGTCAAGGAT
GTTGTGACCGACTACGAACTTTGCATGGGCGATGATCCTGTAGAAAACATCCATTTCCTC
GGGCCGGAATCCCCGGACTACCCCAGTCGGAATGTCTACAACAACAGCGCACCAAAGCCT
TGTGACCGTATTGAAGAGGCCGTCCCCTCTAGGTCATAGATAACGTAATCAAATTGGGGT
GTCATCGTCAGCAGGTACCTCGTCGTAGCTGCCCTCACGTTCAACCAAGCGACCCGTCTC
TGGGTTATACTTGGTAAACACTAGGCCAGTGTTACCGTAGTTCCTGTCCTTGATTACACG
GATTCTGCTCTCGTGCTTCTCTTCCCCGTCAGCCTGCTTGTTCCGCTCGAAGCCTATCAT
AAGCTGACACCAACGCTGCATAGCTCGGGAGCCGGTGAACTGGCTTTCCTTTACCTCAGC
GCCCTCCTCGTGGGTACGATTACCCTTGGGCGGGTTAAGGTGGGAGAAGATGAAGATCCG
AAGACCCAGCTCATCGGCCATACCTGCAAGCTCTGTACAGATACGGGCGATCTCCGTGTT
CATTTCGGAAGGACTAAGGTGGTTGGTCATGGCGGTCATGTTATCCAGCAGGATAGTCTT
CACATCCATGGCTACTGCCCAGAAGCGAATACATTCCTTGATATGATCCCAATCGTTCTG
ACCCTTGTTCTTCCACATGAAGAGCTTGCCACGGAGTTTACCCGCAGCATCTTTAAAGGC
TTGCTCATCCCACTCGATATCCGGTCGGTGGAAGGGAACATTAGCTACCTTCCCGCGATA
TTCTTCAGTGTCATGGCTACCTGCTCTTCCAACAGGAATGTCCCTACGTTCTCCCCGTGC
TCAATGCAATTCCAGGCAGCAATCTCGTGGGCCAACAGGGTCTTACCGATACCAGTACCT
CCACCTACCCCGATGATCTCACCATCACGCTGACCATAGGTAAGGTTGGTTAGACCTTCC
AGGGATAGCTCTTACCCCACTTAGGTGGTTCTAGCGCCTCAGCGTATACCTCATCGACAG
TTACGGAACAGTCCGGAGACTTAACCTTGCTACCGAATCGGATCATCTGGTAGACCTCAT
CCCCGATTACGCATAAGCATGTCGTTAGCATCCTTCAGGGGAAGCTCTACAAACTTACAC
TCGGGGTACAGGGCACGTACCTTCTCTACTGACTTACGACCCTCGTCATCAGCATCATGG
CAATAGATGACCTCGTTGAACCGTTCGAGTACACCAGATTCCTTTAGGAATGCGATATCT
CGTCCAGCGGAACCAACACCAGAGGACCAGCTAATGGCTGCCTGACGCAGATGCTTCCAC
TTCTCGGGCAGCTTCGACTCCATGATCTGAGCCACAGACATTGCCGACAGTTCATCCTCG
CAGATGACTAACCGCAGGTGGCCCATATCCTTATGCCGAAGAGTGTTATAACCGAAGGGG
TCAACACCACCCTTGGGACTACCACGATAGTAGAAAGCCTTGGGGTCTGGGATTCGGACG
TTAAACGCTCGGTACTCCCCATCGCGGAATGGATAGTAATGCTCGATAACGTCCTCACCA
TTTTTCTTGTGATGAACCAACACGTACCCGTATCGTTCGGCGATTACGTTTGCTTGATCT
TCCGGTATCGAGACTTTAGTTGAATACTCAAGGACTTCGTTGACTAAAGATGGATGATAT
CTCTTCAGTTTCTCCTTCGTTCAGAACCTTGAATCCTTCTTCGAACTTCATAGTGGCCGC
AACAGTTTAACTACCGAAGCGGGTACCTTTCTCGCTGTCTACGACAAGATGAGGTGATTA
CCTGTTTTATCCCCACCTTTAGCTCGGCAACCCGGACAAAGCCGTATCACCGATAATCTG
CTTCACTTATTCCTCCATCTGGCTCTTGAGTACCAAGCTCGGTACAGCATTGACGTTACC
CGGCAGGGGTCTTGAGGAGTTGTTACGAAAGTACCGTAGACCTTCTCCAGTTCACGAGTC
GATCCTCCGATCTTCTTCAGTCGCTCCACATGAAGCGAATACCAACAGACTCGTCACGTA
ATTTGCCATGGCTGGTCTCCGGGTTATCCGAGGGACTCCGGAATCTTGGAGACCTCGATG
GAGATCTTGGAGTGTCGTTGGGGCGACAAGGCCCAGGATCTCCTTGAGGGAGCATCTTCT
GGAGCATGTAGCTCTTAAGTCGAAGTTGTTCGGGGCAATAAGGTAGACTTCTACTGCTGT
CTACGGTCATTTCATTCATTATACTGCTCCTTGTCGTTTTGCCAGGTGTGCCTTCAGTCG
CCATCTTGGACGATATCCATCGCCATATGGCTATCCTGATGGTACACCAGAATGATTCGT
TCCCCGATCCGGAAATCCTTGAGGGTACGATCATACCAACCCGGATAACCTCAGAACGTC
AAGTTCTCCAACATTTCCTCCCCCTTCTCACCTTCGGAGTTGCCAGATTTCCCCACTACG
ATGAGATCGCGCGGGAGGCAAAGGAGAGGATGGGGAAGCCTCCAGCGGAGTACACATCAG
ACCCGCTCGCAGGATACAGCATTGCCGACAAACTCGGCCCCTTCCAGAAGAGTGGATGGT
TGGTACAAACCAGACGGGGAAATTACCATAAACGAAAACACGAACAACCATTAGATAGTC
TCCTCGAAGATTTTGTTGGAAGCCCTTGACGTAGGCCTCCAGGGCGCTGCCTTCTGCATC
AGGCCCGGCGCGGTGTTGTTCCAGGACAAACGCCCGGCTAGTAGCGTGCTACGGTGATGA
TATCCCTCCAGCGCCGGGGGGCCAGGCTATACAACGGATGGCTTCTGCCGCCGCCGGCAT
AGCCTGCTCCAGCTTCATCGATCCATTGCATCGTTGACGCCATAGACCCAACCGCCCGCT
ACGTTACGTGATGCTTTGGCCTTCGTTGGTCACCATATCGTTGGCAACGGCGGAGCTTGA
CCTGGAGCAGGGCAACTGGATCATGGACTCCGGAACTCGGTACGCTTACCTGACGATGCC
CTGGGTGAACAGTTGGGCGTTCTCGACGACCTCCGGAATGGTATGTTGGATGTGTACCGA
GGTAGTTACCCCTGGCGACGAAGGGCGTAAATGGCCTGCACCTCTGCGTCGTTGATGCTA
CAGATCAGATGAATACCCTGCCGCTATGACCATTGAGGTCGGTACGAGCGTACATCCGAC
CACAGGAGTCCAGGATCGGGTGGACATTGGCCCAGGAATCCGTCCAGCGGGGATCAGATG
TGGGGCGTTGGCCTCGAAGTGGCGGAAGAAGTCCAGCTTGTTGGTGCAGCGAGCAACCAT
GGCGGGTCATTGAGGAAGACCGGGGCGATACCCTGGAAGGCGTCATTGCCTCTGAACCTA
CAGTACCCCAGTTGATTACCGCAGTACCCGCGGCGTTGGGGTGAGCCCCTCGGACCGAAG
GATTCGGGCACCAGAACGTTGCAATTTACGGCACCGGAAGCTCGGGAACGCTGGCAGATG
TAGAGCATGGCATATCTCCCTGTTTCTGTTGGGGTAGTAGTGAAGCCAGCAATAGCCCCT
GAGGTTAAGTTGAACCCATGTACATCGCCAGGAACCGGCATGGGTCGGCCAGGGGCTATT
ACTAACGCACTACTTTTACTCAGTAATCTTCGAAGAGTCGATCACGATGGCCTCGGCAAT
TACCACCAGGGCAGGTACAAGGAATACCCTCGTCGGATTGGCATCGTTGAGCTCCTGTAC
TCGCCGAGCTCGACGGATCGACTGGATCTTGTCCAGGGTGTCGCGGGAGAAAAGGATACT
TACGAGTTGCGTCGGTTTCCTCCCTGGATCACTACTTGACGCTTGAGAGTTACTTGGGTG
AACGGCCTCCTATTTATTCGATTACGCATTCGCCGCAGATGAACTCACACTCATCCAGGC
TACCGCAATGAACAGCACCCTCGTACCGTCAGATGACGTTACGCTGGTCATACGCGGTAG
GGATCTTACCACGGGATGCACCCGCTTAAGGACATGAACAACGTTGTTGAACGTGTGTCC
CTGAACCTTCAAGGGTAGCGATGTTGGCCGGAGGGTATTCCCCAGGTGCCGCCGACTTGA
ACCCTGGGTTTAGAATGGGATTTCGTCTCCGATGAGATCGTTCAATCCTCAAAACCGGGG
TAGTCTCCCCAGGAGTCGGCCGATGCTTTGGGTCCAGGCAACTTACCGCCAGATCCTCGA
CAGTAACCTTGAGATCACGGTCTTGGCCGATGAAGTAGGCGTTGGAGATACTTCCTCGTA
GGACGGTGTAGCCGTGGACTTTGGCGACATTGATGCCATGAGCTTCGATGAGAAGGTTTT
CTTCACGCTCGATTCCAGTGATAGTTCCGTACTCGCTGCCGTTTGCGTAGGCCTCGATCT
TGACAACATCGAAGGTGACGAGCTGCCGGTGCTTGAAGGTGCTACCTGCTGCATCGAGAT
TGTTGTTGATCCGGAGGGTGTTCCTTGCGTACGCATCGCCAGACGTTCCATACGACGAGC
CCGCTTTGCTGCCTTTCGTTCCCGCTTGGCTGCCTTGGCTGCATTTTATGGCCCTCGTAG
TCGGAGACACCGACGTAACGACCCACCATGCCGCTGCTTGCCTTTCGCTTTCGGGGATGG
GAAGGTCGAAATGTAACCGAGTGGTCTGGGGTTCTGCCTACTCCTCCGGCCTCCGTGAAC
GGGATAGTTACCAGGACTTCAGCCTTCAGTTCAGCGATGGGAGAACGAAGGCGGATCTTA
ACGTTAAGGACGCTCCAGGAAAACCTTGAGGCATACCAGGCTCCGGATGCCCACACGATG
GTACCAGTGGTGGTCACCGCCATGAACAACGGACGCTCATCGTTGCGGATGAAGTTCAGG
GTACGTTCCTTGAGTCCCACCAGACAACCGCAGCAGCACCAGAACTTCTCTCGAACGTCT
TCTTAACCGTGGTTGGCAATGGTGGCGCGCACAGCATGTTCGAATCCACGTTGTACTTAT
CATTGCCATCCAGCAGGTGCAGGCCCCAGGTATCTACGGTGCCGTTATGTACCATGGTGA
TGTGGTCTACCTGGAAGGGTGGGCATTCCCATGGTCCGTCTTGTCGCCCATCGTGGCGTA
ACGGTTGTGACCCATGTAGATGCGAGCCCGACCCTTGTCCAGGAACTCCTTGGTTTCAGG
ATCAGCCAGGAAGTCGTAGGCATCTACTGCCCGCTTGTGAATTCCTACCTCGTTCTTAAC
CGGGTCGATCTTGGCCAAACCGGTGGCGTGAGCACCACGGACAATATCCACGGCAGGAGG
ATTTGAGAAAAGGCGATTTCGTTATCACTCGCGTTAGTTGTGGCGCAAGCCTACCAATCC
ACACATTGTATTTCTCCTTAGTACATATCTGGTTCGATTGTTACGCCGGTATGGCGCTGA
AGATTGAGGGTTATCCATAAGGCGCTACGAAATCTTGAAGAGCCTCTTGGTCACTCCAGA
AGGTCTCGTACACGCGGTGACCAATACGACGCAGAGGATACCGCAGTGATCGCTGTATCA
GTCGAACCTGGGTACCTGGCACCGTGCCAAGTGTACCCGCTCTGCTGCACCATCGAGTAG
TCTGGTGGTCGAGCTAGCAGCAATACCGATCAAAACCCTCTGACGCTCACGTTCCTATGC
TCCGGAGACGTCGCTGACGGTTCTGTTCATCCTCCAGAGGATCTACCCCAGGCGAACCAA
AGCCCATGGTTGCCACCGCGGTAACTGGCGATCCCCATTTCACGACCACCCGGAGACATT
TCATAGTCCGGAATGCCCCAGGAAGGCACATCCCGGAGAGATTCACCTCGGAAGCACGGA
ACTCTTCCAGGGGGTTTCTGCGTGAAGCCATGGCGAAGGCCTTTAGACACAACATACGCT
GCATCAGGCCTACCATTTCCTGGCAGTAGTCATGGCGTGAACACCACGAGAACTCGATGG
ATCGAAAGTTGTGGAGTTACCTGGAGGTTCAAGGCCGAATACTTGGGCCAGGGTAAGACC
CAAGACTTTCTCTCAGTCTGCCTTCCGGGGCCCGGGATACGGCGACCGAGGTCTGGAGCC
AACCAGTCGTTGCTGAAGAACGGGATGCAGGGGATTCACTATCCCGACGATACGGCTACA
GCGGTGATCGAAAACATCCTCGAATGCCATGTAAGTCAGGACCGTTCGTTCGTACACCGG
CCACTCTACGTCCCGCATATCCATGTGCAGGTGGGTGGAGCAACGGAAGGTTGGGGCCGG
GGAGTTTCCTCCATCGCACTCACAAAGGATTCAACCCGAGTGATTGCCTTCTGGCCAGAG
GCGGGACCGAATACATGCTCCATACCATCCCTCAGAGATCGTCGCCCTTCAGTCCACCCG
TCCACTTCCGGCCAGTTGTTGAGGCCTTCCAGTTCGACTTCGAACCCGAAGGAAGCAACC
GCAGGCAGTTCCTCGTGGTGTTCCGGGGAAATGATCTGGTTCTTACGGAACCCTATCTGT
GTGCCAATAACTGACATAGCAAGTCCTCCTGGTTCTTGAATTGTTCATCTGCGGACAGTC
GGCCATCTACCATCTGACCAACCTTCACCTTCCAGTGAACTACCCCATCCTTGATGAAGA
AATCCCGGTGGGAGGGCAGAGATCATTTCCTCGGCCTGGGTGAAGATTCGGATCATCATT
TCGCCGGTGATAGCCTGTTGGGCATGGTTCAGCAGGATGTTCCTGGGGTCCACACCCTTA
CGCTGAGTCGGTTGTTCACGAGGAAGGATACATAGGTATCACGAACCCACGGCCAAGACG
TCATAGGTACCCACTGGAGGACACGCTCTACCTCGCTGTAGACAGCCCACCGTAGTAGTT
ACCGCCGTCGAGGGCTGATACACGTTACCGAGGAGACAGAAACCCATCAGATCGACGGTC
GTCACCAATGGACTCGGTATCGTCCACGTACACTACCAGGCTCGCCGTCCCGCGGAAGAG
AGCATGGAATTGGAGGTAGTACATGTTGAAATCTCGGACAACCGATGGCATTCAGGTCAT
TCATATTTCACCCAGCGCTTGAAGTTGCAGCTACCGCCGCAGCGCCGTCGTTCTCGTTGA
TGATTCGCTGGACTTCATCGCCAGATACGATGAGGACGCCGCTCCTCCAGGAGGTGCTTG
GAGGTGTAAGCCTGTACTGCCATTCATAGACCGCGGTCAGGTCTGCGAAGATCAGAAGTT
CGACAGGCTGCGGTATACCGCCGTAGGGCTTATAGCGCATTGCACCAGCCTTGCCGTACA
GCTTCTTACGCTGCGTTGTCTATCGGGATGACAGACGCCAGTCCCAGGAGGTAGTCACAC
ATCTGCATGATGACCTCGCGGGTGTTCGCCAGCGACTCGCTCGATGTGGCTGAAGCCGAT
ATGGACGTGGCCGCCAGCGGCGGTGCGCGCAGGGTCGGAGGTGCATCCTTCGGCATCGTT
CTGACGACGAGTCCAGCAGTTGTAGTCCGGCTGCAACCGAAGGCAATAAGCCTGGGGCCA
ACGGAGGAGTTTCCTCGTCGGTACAGGTGAGACGACGGAGTTACCAAATGATCAATTCCA
TGCCGAAGGGTTGATCCTGGGCACCAAAAAAGTCGGACGGAAATGATCGCTTGACGAACT
CTTCCTTGGTGCTGGCCGGGTCGATGTTGTACTCAGAAGAACGTTGTCTTCTTGGACGGC
ACCACCGGCTACAACCAAAGAGCGCTTCCTTGGTTCCACCGATGAAGCCGATGACGCTCT
GGGGGTTACGGTCGTAGCCGACGAAAACTTCGGGATCTGCACCGATCAGGGGATTGCGTT
GTTCATTGCATGTTCTCCAGTTTTCGAGGATTTCGGGAAGGTCGTAATAATAGTGGGTAC
CGTGGGGAGGAATCTCGTCCGGGATCACCTAGATCGATAGTGATGGGGAGCCATGATGGA
AGGATTCGATCATCGTGTACCTCATCAACCCATCGCACTCGATCAGGTAATCGTCCTTGA
GTAGCCTTTCAGAGGGGATCATCATGGTGCTTGAGCTTGTCCTTGAACTCTTCTCGAGTT
CGTCCATGGATACTCTTGATATCGAAATAGGTGATATTCTTCCACCCACCTGTCAAGTGG
TCCATGATCTCACTGATGTAGCTATCCTCTACATCATCCCCATTGGCTTCGCACTGCTCC
TTATACTCCCAGCGGTCACGGGGAACATCGCCGGATAGCTGTCGCAGTGGACAGTGCTAC
CGACAGACACAACCGCGAGGGTCAGAAGTTCTGCGTTGAACCCCAGGACTTTATCCTGAG
CTTCCTTTTGAAAACGACGCATCTGATCCCCGATACCCCAGAGACGATCACCCATACGGA
GTGCGCAGATGATTGCCGATGCGTTCTTCTTGAGGTCCACACGAACGTACACAGCCTGTC
GGCGGCCAGGGGTGCTGAAGTGGTTCTGGTCGGAGAGGTTTTGGTCTCGTCAGATCCTGT
CCACCACTTGGTTGGCCAGTCCTGGGAGGCCGAGAGGATTCCGCCATCGGTGATCCTGTT
GACCAGATCACTGTTCAAGAACTCCACCAGCTTACCGGCAGTGTTCTCATGGGTGAATAC
CAGAAGGTGTCCAACCCCGGTTTCCCACACTACATCGGACAGGCCCCGTTTCGTCTGGGA
AAATGCGCAGAAGCAGTTGGTATATTCAGCCATTTTGTTAACGCAGCAGTGACCAACCTC
GACATCATGAGTACGTTATGAATGCATAGAACATTTGATTTCTCCTTGGTTTGTCAGAAG
TTTTTTCTCGATGAAGTCTTCGAGACCCAATTAGAGATATCCTTTATGTCACGACTCCAG
CCAATCTCACCCATGATCCCCAGCATGACCACTCCGTATTTTTTCACAGGTATGAGAATA
AAGGCGGATGAAACCTCCACCTCGCTATCTACGGTCATGTAGGTTCTTTCCGAAAACGGT
TGGCTGAGGGGCTATGGTAACCACCTCCACCTTGTCCAACTGAAGATCTTCAGGAATTCG
ACCGCCATGGTGTACTCCTTACCCACTCACGTCCACCTGGAACGATTGGCCATCCAACGA
CCGAATCGCATAGACTCTTCAGCGGATTGGATGTGGATATCCCCTTCTTGAGATCATCCC
AATCGCTACTGAACATGTAGTTGGAGAGTGGGTACCCGGTGTAGCCATTGGCTATCAGTC
CGTGGAAGGACATTGCGAGGTAGGACTTCCAGAAGCCATACTTCTCAGCAAGCTTCAGGT
GAACCTCCTCTTCACGACCTTGGTAGTCGAGTCCGCTGGAGATCATCTTGAAGAGGAATT
TCATGATGGGGTACCAGGAATCCAGGGTGTGTACCTTGGGGTTGAAGTAGATACCGCAGA
CCCCTTCTTTTCCGTGATGGGTGGAAATCACTTCCGTCCCGTTAGAATACTTCTCGAAGG
GGAATCCTGGTCTTGCTTTCGTAAGACCAGGAAGTGGACCTTCGGGATTTTGAAGGCCTT
GCCGCCGATGGTGATGGTCTTGAGTTCACCTTTGGGTAACACTTTGTTGAGTACATCGAT
GAACTTCTTGCTTCGGCGGGGAGCAGATGAACCAACCGTCGAGCTTCTATATCCGTGGGT
TACCGACCTTCCATGGTACCGAAGCAGGAGGAAGAGGTCATTACCTTGAGGTGGCTTTTA
CCCTCGCCCTTCTTGACTTCATACCGAAGGCATACTCTTGATACGGGCCATTTGTTTTCT
CCTTGGTTGAGTTAGCCAGCAATACCCCAGGACAGGAAATACTAGGGGTATACAGCGACC
TCTAAGTTACTTAGAAGGTGCCACGGCGCTGTTGCTGCCTTTGGTCGGTGCCGACACCTT
GGTGGCCTTGAGGTTGCCGTTCAGGGTCAATTCACCGTTGGAGCAGAGACGCTCATAGAA
ACCTTACGCTTACCCATTGTTCACCTCCTTAGCGAACGTTGATGAAGGGCATGGCTTGGC
CGCCGGTGACGTAGGTAGGCAGCTTGCCGTCCCACTTCTCGATGGCATTCAGTTCTACCA
CGCCGGGGTTCTGGCGGAGGGCTTCACCCTGGATGCGGAGGCTTTCGGCCTTACCTTGTG
CCACCAGGATAGCCGCTTGCTTCTCACCCTCGGCTGCCGCGATAGCCTTAGCTGCCTCGG
CCTGGGTCTGACGCAGTTCGTTCTCACGCTGCATCGCCGTCTGAGTGGCCTTGATCTTGG
CATTGATCGCCTCGACCACCTGCGCCGGCAGACCGATCATACCGTTGAGGTACAGGCTTT
CGACGATTATGCCACGGGTGGAGAAGTGCTCACGGACGGCAGCTTCGACTTCCTTGACAA
AGGCTTCCTTGCCAGGACCATAGATGGCCTCGGCGGACTTCTTGCTGCTGGCGTTGTTCA
CGGCGTTACGGACCACCTGCGGCACGTTCACGCTTACGATCTCGTTCATGCCCTTGCGGT
AGGTCTGGAACAGCAGGGGTGCGCTACCCGGAGCAGCCCGGAGGTGACGCCGATGGGGAG
ATCCAGCTTCAGGCCGTCCTTGTCCTGGAGCGATACAGATCGAAGTTCTGGTTGAAGATT
GAGACGAACAACTTTCCTCGTTCAGGTCAGGAACTTCCAGCCGACGGGGTTCCTTCAGAT
CGACGCCGGTCGCCCATCATGTTGACGACGACGCCGAGGCAGCCAGCGGGAACCTTGGAG
CAACCTGCGGTGATTGCGGGCAGCTTAATGGCCAGGAGGCCGAGTTTTCTTCGGTTTTGC
TACTTTAGCCAAGAAGAGGTGATGTAGACCATGCAAAGAAATGAACATGACGGGCTATCA
ATTTACATAGAAGCTATCGTAGGGAACACCAGATCGGCAGAGGCCTTCTATATATGTATG
TTGTCGTAGAGGTCAGGGTATCTTCTCCGATGCCCTCTAAATCCTTTGGATTGCCTCACG
GAGGCGGCCTCGCATCTGTTCCGTCATAGTACATCAGACCTCCACCGTCAGGACTTCACC
GGCTGGCAGTACATTGAAGAACAGGGACAACTCACTTGGCGAACACGCCATGATCGAACC
ATCGCTGATATTAATCATGGAAATGAACCCACGAGCAAGGGATCTTTCTGGCCGGATGTA
GACCTGGGAGTTCTTCTTACGTTCGAAGTACAGCGGGAGGGTTGGCTTAACACCCCTCGT
TACCTTAGCCATACTACACCCCTTCTTGCCCGAGAGTTTACCGGCGTAGGAGTACAGATG
GCTGACAGCCCAGAGGAGTACAGGATGGAGAGGACCAGGAGGCCAGCACCCATGGCGTTC
ATCAGGGTGTTAGCCGGGGACAGCATCTGGGTACTACCCAGAGGATGCTTACCCATGCGA
TAGCCAGAGCATTAATTCGAGTGTAGAGCTTGAGGACCATTGTTCTTACCTCTTTCTTAA
GATGAAGAAGATAGTAGGTTAGAACCTTAGGTATCCTAGGGATATAGCAACCCCCGACAC
CTTTTAAGGTTATTATACCATACCTTCCAGCAGAATGTCAAGGCCTAGATGTTACAGACC
TTTACAGCATTTTGTCGAGGTACCTGAGTGCTCGACGGTACTGGTGAAGTCCTGCAAACC
ACCTAGGTGGATTACGGAGAACCCCATGGCGATGAGGACTTGCCGCAGCCTTCACGTTGA
TGACGTGGGACCCGGACCTTGGTCGAGTGGTTGTCGGATGCCCAGGGTTGTAGTGATGCC
CATGCGGCGGAGGATGGTGTCGAGCACACCTGCTCGCTGTTCACCGTGAAGACCATCGTA
CCGTGGCCCTTCGTTGATTCACGGATAGCGCCGATCAGTTCCTTTGGCGAAACCACGGGC
TGAACCCATTGATTATGCCGCGTATCGGTCAGCGGTACCCGAGGGTCCCATGCCGATGTA
GATTTTGCGGTACAGCAGGAAGGAAAGTTCTGCGGTGCATTAGTAATCTCCCTTTGATTA
ATCGTTTGGTTGATAAATCTATTCCGTCAATGCCAGAGCCAATAAGCACCGACACAGCGC
CAGGAAGCCCAGGGGAGTATGATGGTTAGTAATAGGAAGGCGTCATGCTTTCATCACCTT
CCGCTTAACATCACCACAGACTTTACACTGAAGGTCGTAATAGACAGACGAGGCTACTTC
ACCACCCCTTTGTGCATGTACGAAAGGACCAGTCCTGATGATCTTGTGAAGTCGTGGACC
CTGAAGAAACAGCTGAGACGACCGAAGATCACTCAGTCCTCGCGCAGTTGCAACAACGAC
CATCTTTGGGGCCGAAGACCGGCATACAGTGGGGCAAGGCCTCACCAGATTGATCATAGC
CTCTGGCGTGGCGCAGGTAAGCCGATCGGCTGGACTCCATACCTACGGCTGCCAGGGTGA
AACCAAGCTCACCACGGGGCTCCTGGGGCCTTCCTGGAGGCTCATGTTGAACCCCGCTTT
TCTACCCGACGCTTCATGCGGCGCTCCTGAGCCATTATCTGGCCTATCATGGGTTTATTC
TTCATACGGCCCCACATGCAGGCATTCACCGACAACCACTTGGCCGGGAATAGATCAGGG
TGAGTACCGATATCCTTGCGCGAGGATGTTGAAGCCATCCTTGTGAATAATGGTCACGAG
CTCGCCCTTCTTGAGATAAACCAAGTCTCCAGACCCGGAATCACCCGAATGGACGTAGGC
ACCGTCCGCCATCTTGCTGATATGAAACTCAAACCTTGGCTTTGCCACCGCTACGGTGAA
AGAGGTATTGGTCATGGCGTCACCTCATTGAACCCGATGAACGATGTGGTCCACCGGGAT
TCCGAGTTCGAAGAAGGTTTCGGTGATGAACTGCCGAAGTTAATGACCTTTACGTGGCGT
TCACCGTCCTTGCCACCAACCAGAAACTTGGGGTTTAGTGGAGTTCATATAGATGATAAT
ACCTACACGTTTGCCGAAGATAGCTACCCGGATGTGGGTAACGGCCAGCCGGGGTACATC
TTGGGGTTAGGCTGGAATCGCCGGATGCTGACCAGGGACTCCAACCAGAGGAACAAATAC
AGGGCGGGCGTCATAGTACACCTACCTCACGATGCAAAGGGATGCTGTCCAAGGTCGAGG
ACAGGACGAAGAACCACCCACGCTACCAGCAACAACCCATTGGAACGTGAGCGTATGGCG
GCACATGCAATATACTTCCATTCCATCACCGCGCCCTGTTCTTAACCAAGGATGCCCGCT
TGGCACGGAGCTTGTTCAACTCAGGGTTTTACCAAGGTGGCTACCATTGGTATTGGTAAG
CTCACGAGTTACCTTGGCAATCTTGCGGTCGATAGCTTCGATGGCTTGCATGTTGCGGCT
TACCTTGCGATAGTCGTTGCGTTTCACGGGATAGTTTCCTTTGTTGATTTCCCAAGACTC
CCCAGCCTGCAACCATAGGTTCGGTGGGGTTTCGGCCAGGAATCACCATGGCCTCATCAG
TTGGGTCAGTTGATACCCATCTCTTCCTTCCAGTCACGGAAGAAGAAGTATCCGATGCCT
ACACAAGACACCACGATAGCAATAGTCATCGCTTCAACCTCTTAATCAATTCTTGCTGTT
CTTGCTTCCACCTGGGATCGGGGTGAACCCGCAGCATCCTCGTGAGCTTCTGCAACCGAT
TAACCATGAGTGCCCTTTCGTGGGCCGCTTCGTAGGGTGACTTCATTTGGTCCCACCCCG
CAGAAGCCCATGGGACGGCCTTCCACATAGACGTCTGCAGGCGAATTTACCATCGACGAA
AGCCCAACGGTAAACCACCTGTTCAACCCCGTTGCTATTGTACGAAATGAACGGGACAAA
CGCCCTTTGCACGTAATCGTTTGTAACAGCTACCAGTGTCACCAAAAGGTCCACACGGTT
ATATCCCTCGGTTAGTTCATCCATACAATACACGACCTACCATGTACTCTAGGGATATAA
ACCAAGGGGCGTCATAGTACGCCCCCGGGCCATCATAGGTAATGCCTTTCTGCGTATCGC
TTTCTGCTCCTAGATACCGCCCACGGCTTTACCATGGACAGATACTTTCGGATCTGTGCC
ACAGCTGGTGAGGGGTGCTTTAGTAACCTTGGGAAGAACCGCGCCACCCACTACCGCATC
AGGCCGCCTCGCCGTTGTAGAAACATCTGGCGCAGCAGGGTATTCAGCTGGATCTCACCT
GGTCAGCAGTGACCTTTGACTGGTCCTTGAACCGCTGTCCAGTTCCTTGTTGCAGTCCTA
AATTGTTTTGCAGGAGCCGTGCTTGAAGATCGATCGGCTTGAAATTGTCAGCGGCGCTTC
CGCCGGGCCGCAGTCGGACCAGTAAGTGCTCAGGGATGCCGGAGAACAATGCCGCGCCCT
CGGTTTCCTCTTGGCGAACGCCCCAGGCGCTGTTTGTTGCCGTCGAACTTGAATTCTTTT
TTTCAGTTCCGACCACTTGATCGGAGCGTAGCGCTCGAAGTAGCGGCGCAGGCTGTTGGT
CTTAGCGCCCTTCGGCATCATGGCCAGCATCGCACGGATAGCCCGAACATCACCATGCTC
GTGAGCCAGTACGGTAGCCGAGGCCGCCAGTTGTTGGGTCGCTTCATTCCAAGCCTTGTT
GAACGACTTCAGAGCTACCAGACCAGCGGTGATCTGATCCTTGCCCAGGATTTTGTCTGC
GGTGAGTACAGTTTCATAAGTATTTCCTTTTGTTTCAGGAGTTGGGAGAATGTCAAGGGG
TTGCCCAGACGTGGAGCTACCAAAGACCGACTTCCGATTGTTGCGATGGATATCAGTCTT
AGCAGTCTTGCGACCGCCTTTGTAGACTGCGGTCCTTTGTTTCGGTTTGCCTTTCACTTT
AATTCCCTCTAGAGGATACATTGCAAAGGGTTAGTCCTTTGTAATATATCCTACCTAACT
CCGAGGTTACTTGTAGAGGGAGCCGGATGGCTATCGTAGGGAGTAACCTTACCTGTTAGG
TAGGGATGCGTCTTACCCAGGACTCTCGCATACCGGATCGATAAGCTGGTAATTGAGGAT
TCGGATGGCTACCCTTTGACCTTGCTTTTCAATAGTCTAACCGCCGTTCCTGGGTCCGCA
ATCAAGCGGGTTATCCTGGGGGGATTGGTTAGACCTAAGAATCGGTTGTTGAATGGTCTA
GCTTAAACTATCGGCGGCATTGCAATGCCTGCCTACCAGGAAGCTTGTATCTCAGCTATC
CAGCGAAGATGGCTGGGGACGAATCGCTATGTTAGAGCCTTGGGCTTGGCTTGTCAAGTG
GTCCGTCTGTATTTACGTGGTGGACCAATCCTATCGGCTGGAGTCACAGCTTTGTAGTTA
GGCTATAAACAAGGCGCCCTATCTACACCGCCCGGACCATCACGTCCGCTTACTGTCAGG
CTACGCTAATCCCGAGGAGTCATGCACGTTATGCCGTTGACTTGGGGCCATCTTACCGCT
TCCCCTATCCCTTGTCAGCCCCCGATGCCTGCCGGTCTATGCCCCAGGTGACTTAGGCAT
TCCCCCCCTTGGTTGGCAGCCCCGTTAGTGTCCGTAGACTCGGGGGGGATGGTGGCTTCT
GGGGTGGAACGTGGTGGCCGATAGGCCGGGTTCGCGGGGTTGTCAAGCCCCTAGGGGTTT
CCCCGTTGCCCTATCGGTTTTCCCTCAAGGCGGCGCCATTAGGCCATATTCTTTACACAC
AGGGTTGTCTACTGGTTTTCATACAGTCTTGGACTATCGGTCCCCAGCAAACCCCGGATA
GACGGTCTTTGATTAACGCAGGGTATCGTGGGATGTCAATGGGGATCAAGGGGTTATCAG
GGATATTCCCCGATCCACCCCCTGAATACCTAAGATTTTTTCTTATAGAATACCCTAGGG
TCTATCCGATGAACGGTAGCTCGGGGTTGACCCTGAACTAACCTTATGGTATCCTAGGGG
AGGGGCTAGGGCTATACCCTGAGGATAGGCCACCCGCTATCAGGGGGGC

In another aspect, the invention provides an TIVP-27 bacteriophage targeting Pseudomonas aeruginosa. In certain embodiments, TIVP-27 binds a molecule of type IV pilus. In certain embodiments, the TIVP-27 bacteriophage has a genome comprising a nucleic acid sequence comprising SEQ ID NO: 5.

TIVP-27
(SEQ ID NO: 5)
CAGCCGCTCTCATTTTGCAATGAGAATTCGTACAGTTCATCATGCGCCTCCTTGAAAGTG
ATTTCTACTTTCGGTATGCGGATGCATGCTATATACTTGTATAGCCCGCTATCCCGTTTT
GCATGGCACACGAATTGCATAAGCATGACAAGGGTAACCTCGAAAGCGGAGCTTTCAAAG
TGGTCTGTGAAGTGGTGGCTCTACCTCTATTATAGTATTTGCGGACCGGAAGTAGTGACA
CTGAAAACATTGACTCTGCAGCTCCAGTAAATACGTTAAATCGAAAGGCTGATAGCTGCC
GCTATCGAGTAACCAACATTCACAATAAGGAGTGCAATGAATGACAAGCAAACAATCATG
CCAAGATCCATTACGCTAAGCAAATCTACTACGCCATAAAACGTCATCGCAGGTATGATG
AGATATGCAAGGAATGGGTCACACAGGCTTTGAAGCGTTCTATGATTGGCGGTGAATGTC
AGGGCACGCTACCGCCTAGATAAGAATATCCTCTTCCCGGGAACAAGCCTATTCGCATAC
GTGCGAGTTCACGCTCACATGCATACACTCTTCACCAACTGTGACAAGACAGCGAATAAC
CGTGCCAATACGTGGGATAGTTATATCCCTTAGGCGTCCTGCCTAGCACGCTATGACAAG
TACGGGATGCTACACGTTGCTCAGTGGTGACACTTTGAAAGAAGTCTACCCTGATCGTTC
GACGATCCGATGTGTTTTCCATGGCATGGCAACATGCCGCCATTAGCAGCCATCCGCGAA
TGGCTGATCTCTATCGATGGAGGATGTCTTCACAGTGTGAACGTTGTTCGCTGCTGTTGG
AGAGAGCGGGATGACTTGGCGCGACATTGCACAGGGAAGAGAGACAACCCTTTGCAACAA
CATTTGATAAGTCGATGGTCAACCCTTCGAGAGTGCGAGAACTTTGTGTGGAACTCCGAT
AGCGAAGCTTAATAAACGTATATGCATATTCCATGCCAACATGTGGAAAGATTTCCCCAC
CCGGTGGGCGGGAGCCCGTTGGCATTAGTTTGTATAGCTTTAGCTGTCGCAGCGGAAAGA
CCACGGCCAAATTCATTATGGAATTTATTAGGTTAGAGAGACAGGGTTCGTGGTTAAGCA
GCTCCGGAAATAGAACCACAACTCCCGAACAGGATACACTCGGACGTAGGCCACACTACA
TCACTATTTTCTCCCAGCCAGAGTAAACGCACCTTCAAGCGGTCATTATTTCTGGAGTGG
GGATTTCGTCGTGTGATACAATAGCAGCTTCCGCTTGGTAATAGGAGAATGGCCAATGCA
AATCTATAGGATTGGTATTGGGAATAAGCCGGACAACAGCTGTAGACCGTAATCCGTAGA
TTCGCTGCCCGGGCTTTTGTCTCTACTGATTGTATTCCATGTCCCTGCTTCCAAGCATCC
CTGCATAACCTTGCATACTCTTCGTTAATCTGGTCCATGTACTGCAACGCTCGTTCTGCT
CTACCGACTGAACCCGTTGTGACGGAGAATCTTGGTTCAAATTTCATCATCCCAGAAGTC
TCTGACCAAGCTTCCCAAGCCACTGCTTTTCAGACTCTTTATGCAATCTCGATAGGCTGA
TGTTCAATATTGCTCAGACATTAGACCCTCCCGAACGTTACGATCACGCCTTTGCTTTAT
TAGGGTTGCTTGTAGCTCATCCAGGAGAGGGGCTTACTGCCCACGATGATGATACTTATC
TGCCTCTCTAGAGTCATCACACACTTAGTCTGAAGGTAGGCCTATACAAGGCGTACATAT
CGGGTTGCCACTTCGCTTCGCAGGCCTGTTCCAGAAGCTAATGAACCGTCTTCGAATGAT
CCCGATGTACAGGCACCGGGGACTATCGTCTGGCCCATCTACGGAGATAGGGTGTTCCAT
CCACTCCCCTGATCTTATCAACGACGCCCTTCCTACTTCCTTCAATTGAGCGTACTGGTT
AAAACGGGTTAACGCACCAAACTGTTCGGCAAGCGGAAATGTTTATCGATCTCTTTAGTT
TTCATTCTCTTCCTCCATGCTCCGCAATGATTTTCAACGCTTGAAGCGTGTAGGTTACGA
AGCTCTGTGTAGACCAGTGTCACACTCATGAAGCGTGTACGCGGTCACTTTCTTCAGTGA
CGAAATGGAAGTCTACCCCTTCGAAGTCGTAGGTCGATTTCGAGTATCCAATGGCCTTTG
AGTTCTTTTCCTCCGTACCACAGGAATGTCATTGGACTTCCGTCGCTTTTACGAGTGTGT
AGTCCAGCAGATTATCTCCAACCCACAGTAGCACTTTTGAGCCTTGTCGTACATGAAGCC
GTTTCCGCCCCAACCGATACCATAAGCCTTAAGAGGTGCTGTTGTATCGCAATGTTCTCT
TCGTGGGGGGTCTTCTAACAATAGTGTAAACTTCACCAAACGCTCTTTCACCCTCTCTCA
TTCCTCCTGTTCGTGCTTTATGGGAGTGTAAACGTTCCCAGTCACTGTCAAGTTTCTGGG
GATAGACCCAGACGCTTCAGCTTTTTAGAATGATATCCCACTCATCGCCTTCTTGGACCT
TCTACATACTTTCCATCATCAACGCACGGTCAATCAGACCAGCCTTGTAGTTATCCCGGC
TACGCTTATACAACAGCTTGTCGATGTAGTCCTTCTTATCTGCCAAAGCGATAGCCTTCC
TGGCCATATACAGCGTATTCTTCCGAAGTCATCCACGGCATATTGGTCAGTACGAGGTCA
GCGGCGTCCCCTGACCAAGGGGATATTCCTCCATAGAACTTTACCGGTATCAGGTTCGAT
TCGCAGCTCTCCACTGTAGCACTGTTCCGGTCTTAGAATTGGTAATATGCACCTTCTCAT
CGATACGGTGGGCCATCATCGAAAATTCCACGTTGCGGCTCCATGATCCATCGCTTCCAG
TCGAAGAATGTAGCACAAATAGCTTTCCAACTGGCTCCGACCTGCAACCGTCTTCTTCAG
ACGTTAATCTGCTTAGCGTTTATATACTTTACAGGCCGGGAGGTCGGATCATAGCACCTC
TTACCTGGAACCTCAGCCCGAGACCCCAACAACACTTAGGCTCATCGTTACTTTTCCTCT
CGAACCATGCGAACAGCTTCTAAAACATATCCAGTCCTCCTTCCTGTGATACAATTTAAA
TAGTCTCCACACTTTTCTAGGAGATTAACGAGGAATAGCATATTCATAAACGGTACGGCG
ATAGGCCATCATTCCAGCCAACCACCGCTTCGGGCCTCTTCTACGCTCTCGGTAATTTTC
CCTCCACCCAAGAAGGGGGCGTAGAGATTCCCATACGAACATCCCTATCACAGAGTAGAA
CAATAGGCAGACCACTCCGGACGACTATGCATGGCGAGAATGAGAAGCCCCACGGTCTCC
ATGGTGCGGATCCCTCTCTTTAAGTAGTTTACGGCTGTTCCAGATAGACAGAGCCCACTT
AGGTAACAACACCACTTTCAAAACGCGCTGCTGTATGTTGCGTAGAGTAGTGCAGTTAGC
ACACTGCGTATGTATTAGGCCATAGCAGTCCCAGTACAATGGCAAAAGCCCGATACCCTC
TCTGTGATCTTTTAGTCGCACTAGCTGGGAAGTTATCTTTGAAATACTCCGTGATACCCA
TGTAGGTCCATTACGAATAATTTCACGCGAGCGAGAGGACGAATAGCATTGGGATTATTC
CATTACCCTCCTTTCGAATAAGTCGAAGTAGGCTCACGTAGAAGTTGCACCACGCCCCGC
CTATGGCCCAGCCTTCCAATAGTCTTCCCCTGAAACCTTGTTCGGAGGGTTATTCTCGAA
CATGGATAACACGCATGAATCAGCTACTTCGTCCAAGAGGTGCATTACCGAGTTAAGCGG
CCCGATTCCAAAGGTCTTATCTAGCCACCCCTTAAATCAACCGCACGTCTGGAGACGGTG
ACGCTGGAGTCACTGGCACAGCGCCAATCTTCCGAATGTAGCACACATGTATGGTTCCTC
GTTCTCGAAGAATGCCCGCATGCCCTCCTTGATTAGAGGTGCAATAGAATTAGAGTTCAT
TGTTTTTCCTTCCTTTGAGCCCCACTAGGCTCTAAATTTTTTGGTTCGACCTGTCAACAC
ATCACAACCCAGTTGTACAGGTCAGCCTCGTAAGCCGATGGCAGCGATTAATTCAAGAAG
AACGCATATCAATAGCTGCGTATTTGGTTGCGAAGCTGTTGATGTACGTTATAAACCCGT
TGTCGCCCCGGCTAAACAGGGCGTATTCCCTCAGTCCCATCCTTGAACGTATCTAACGCG
ATAATATACTTGTCGAAGATCACTGCGGCTTCCTCCCGGCTGATGTAGTAATTGTATTCG
TCCTCTTGACGAGGAACGCACGATAGAAGCCTTTCAAAGTTCCCCTTGACCAGCACCAGA
TTGCTCCTCCACCTTGCATGTATAGATATACATGGTGTTTGCTACTGGATAACAGGATCA
TCCCTGGGACGAACATCCAGAGCAGAGCATAGCCTCCATTGGCATGCTTAATAAGTGTGC
AACTACAGCGAGCAGTATTCTGCATCGCGGCCAGAACCCAGACAGCAGGAGCGACCGGCA
CGAATGCAGGGCGAGTGTAAAACGCAATAGCTACGAGTTTCTTGGAAATTCGGCTCACCC
TCCTTCTTGGTTCGTCTGGGGAACATCCGGCTCAGTAGACGAATGTCTTGGATACTTCAT
GCATTCTTAGCTCATCTTTTGCAGAACCCGTCAATCAATTCTCATGTGCGAATTCCGCAG
CCTTGTAGTAGCGATTAGCTTCCGTCTTGCCTCCACTCCGGAAGGAGGTGGCTGCCCAGC
TGCTGATATTACGCGACTCTTGCAAAATTGTCAAGAGCTTTTGCAGAGAGGGATTGACCT
TCGTCAGGTCTGTACCAATCAAGTCTACACCTTGAGAACGTCAGTCCACCCACAGAGGGT
AGCTCCCCATCAAGCTTCGACCATGACTTCGTACATTTGCTGGAACAGCGTTGAAAGAAA
AGGGCTTTTCGCCCTCTGCTGGTATCTAGAGGAGGAACAAGTCTCTTCTTCCACCTTGTG
AATCAACCGATCAGCATTCGCCCTATCCGAGACTTTTAGCTCCTCGATCTCTTCACATGT
CAACATACGGAGGGCACACCTTAGATTTCGATAGTGACGACGCTCTTCGCATGTCAGGTT
AGCAGGTCCATACCATCTGAACATGTTGTTGATATCGTATCGAATCAACTGAACTTCTGC
CCGTCGCACATATCCACGTCGAACCACGCCACGACCCATCTGACGTAATCTTCAGGTCGT
TCTCTTCGCCATCTGCACTCTAGATCAACGTGACTGCCTCTAGAACGTAAACGTCTTCGA
ACCCTGTACCTTTTTTTTTTTTTTTTTTTCGTTCTTAAGTCGGATGATTCGAGCGCGAAT
CCATTGCATGCACCTGCGGTTTGGGTCTGTGATATCGCAGACCAATGAACCTCTGGAACC
TCAACTGGCATACATGATTGTACCTCTATTTGTTTTACAAGAACGCCATGATTCTGTCTC
TGTCTGCATGGCGGTACAGATTGAAGCAAGGCACGCCTCTAGAGGACTAGCTGCCAAGCG
GTCCTAGTCCCGCCTTTCTGAATCCCCGTTGTGGTCTGTCTTGTTCCAGCACACCAAGAA
CTTCAGGCGTGTTGAGATCGATTCTCAGCACTTGGAAAACGTTCCGAGTGTGGGCTCGTC
TTGCGAATGGACTCAGAACAGGCTCTCCATCATTTCTGACAGAGTTCCGTGCCGGGTGTA
TCCGGCTACAATACCCCAGCCTTCCCACTGCCCGGCCACTCTTCTGGAAGCGATTAGGAA
GCTCCTAGAGTCTCAGCATCACATACTGATTGGACCCGCCTCCGCAGATAGATTTCGTCG
TAGTCCCTCCTCTTTTGTTATTGGATATATAATCGTTGCCAGCCTCGAACGCTGAATCTG
CTCCCTTTCGTCCTGACCGAAGAATAAAACTCGTTCCAGTTTACGACTCTAGCTCTTCGC
GAGAATGCCCCAATTCTTGTTAGGCGTGCATTCTACTCACCTCGTATATCATCTCATCGT
CTCACCATTTGTCCCAGTTCTCCGTATGTAACCCAGTGGTGACGATAACTCTTCTTGGTT
GGTGTACTCCACTAACACCGGATAGAAGTTGTCTTCGTAGTCTATACCACCACCTTGCAT
TCACATCCTTCTGGGAACAGTGTATCCATCGCCGTTCATGGTTAAGAGCATTCCCCGGTA
CACGTCTTTTATCCTAGCTCACTCTCCAGACTCTTCAAACTTGAGTCTCAATTTCTCGAT
TATTTGAGCTTGCAAATGGAGAAGTTCGTACGCTGTTGTTGTCCAGATCAATAGACATGT
CACCTTTCCTTCTAGCTTTTGAATTCTTTTGTCCAGCTTAGTTAGATGCTTATCCAGCTT
ATCTAGCTTTTGACCTATTAAAGTTGAAAGTCGAGCCCGGGCCAACCAAGTCCCCTTTAT
ACTTAATTGCACCCCGATCATATATCCTCCTATTACAGTTCGTAAACGTATACCTGAACT
ACGAGCGTCCCCTCGATAGAGGCTCGACATTGCTCCAGTCCTCTCCGGCTAAACCGCAGC
CACCTCGGCAGACCTACACAACTCCGAGGTAGGAATCGCCCGAGCGTCAGCCTCTGCCTG
CTGAACAGAGACATAGTGTACGGCTTGGCCCAGTGCGTCGCTTTTTTTCGAGTGTAGACT
GACCCTCTTCTCGGCCATATTGTATCCGCGATTATAAATCGATCCGTACTCGTTCATTAG
AAGGTCAGGGAGCTTCGAGTTTACCGTCCCTTTCACCGTTGTTTGGTCTGTTCGTAGGCC
TCCGGGAATTGCTCCGATTTGCTTTTAGCAATGTTGCCGCCCATGGTGTTGAAGCAATTA
GCCTGATGTGCAATAGCGTGCAACTTCCTTTTGTTTTTTGAACGCGGCGCAGGTCGCCCT
TCGGTATTTAATCATTTGAACTCTTCTACACTTTGTGTATCTTGATATCCAAAACTCTCG
CATCGGCTCGATGTTATCGTACATCGAGTCTAATGCTGCGAATGTGGAGACCTCAGTACC
CTTTTCCCAAACTTGAACAATCTATGTCCTTATTCTGGTCGGCTCTTGCTCGCAGAAGGT
TGTAAAATAGTATTTAGCCAACATCCTCACCTCCTCTTATACGCCGCTCTTAATCCGATA
ACGGATAGATTCCAGATCATTGTTCTTCAAATCTTCCGTGTCCCATTCTGCCTTTGAAGA
ACGTCCGCAAGGAACCAGTAGCCTCCTCCTCTTCAGATACATTATCTTTCAACTGGAGGA
ATCGACTCATTTTGCAGGTCTTCAAGTACACTTTCAGGAGACCTTAGCCTCGACTTCTTC
TTGCTGTCAGTTTTCGGGTCTTTGAAGATTGCAATGCCTTCACCGTTCACTTCCGTAGAA
GCTTACCGCGAATCCGAAAGTATCCTGGTGTTGCACTGGTAAGTGTAACTGCCAACTCAA
ACACTACAGTTCAGGGAAGCAAAACCTTTCTCCTTCGCGCCTTAAGATTTTAGCTGCTCG
GCGAGTCGTAATGGAATCCCCATAGATCAAACCGATATGAGAATCGAGCATCTTATAACC
CGCTTCATTCACAGTGCCGAAGGTTTCCCACAGAACTTGATAGCTCCTTTACCTGCGCTC
GCCTACTTCTTTCTGAGTTTGTAGCCAATTACATACTCGTATTGATCCCACTCAGTGGGA
ATAACCTTTGAAGTACTTATCTTCAAAGTTCAGAACAAATGCATCGTTTCCATTTACTGC
ATCCATGATATCCCAGTGTTCGAGCGACTCGACAACTTCAATATCACCCAGTCCGCAGAT
AACCTCTACAGGATCACCAGAGTCTGGACGAATAACCAGCTTGCCATCACGCTCCATGAT
CTGATCCTGATGGACGGCAGAATTTCAGTAAAAGCACGCCCCAGAAGTCGAAGGTTCGCC
ACGTACGACAGAATACCACTCGAACTGGGAACTTACCCATCAGTTCGATAAGGAATCGAC
GTTCTGCTTCACGGCCACCTTCACCTTCCCCATCCTGCATTTCTCAATACGGAGATGTTG
CTAGTAGCTACAGCGTGTTCAGTCGCAGGTACGGAGCAAGCTACCAGACCAGTGCGCTAT
ATAGTAGTCCTTTCGGCGTAGATAGCCGCTGCCAAGGTGTCCGAGCCTACGAACGAGGTC
AAGACCAACACCGCTTCTTCGGGCCGCGTCTTCAATCCCACTCATACCCCGGGCACTGAA
GTCGTGAAACTGAACGGACAGCAGGAAGTCGTCGTAATAACCCAGTGTGGTTCGCGTAGT
ACCGGGCCCATAGCTGCGTATTCTGCGGCAATAGGTTGCGTTGGTTGCGGTCTTCCAAGT
CATAGCCGAAATCAGGGTCTCATGGTAGTTCACCAGCCAGTAGAACTCAGGCAGGGGGTA
TTCTGATAGCTAGCAATAATGCCCATAGGAACGCGCTAACGCCCCTCGGTCAAAGACTTA
ACCTCCAGAGGCAGATAGCCGAGCTTGTGAAGGGCGCGAAGTTGCCCACGGCTTCCATTT
CCCCGGCCAAAATAGCCAGTCAGGAACAACCGGGAATACCGGGCCAACGGCGTTTCTTCA
GCCGCGAAGAAGCCTTCCCAAGACTCTACGATCTCCATCCAGCGGCCCGTCCGCCGAATT
TGAGAGTGCCGTCGTAGAAGTCGGTGGCATGCGTCTTTCGATAAAATCTTATCGGAGCGA
GGTCAGGCTGCTATAGACAAGCTGAGTTCCTTTTCCGAGGCACATGGAGCCGTCCAATCT
TTGTACCCATCAGTGGAAAGATGCGCAAATTTTCATGTTATTTCCTCCTGTTGTGTTGAA
TAGGCCCAGACTACATCTCTGGGCCTCCTGTCAATATTCGACTTCTTCTACTGGCAGCGT
CAAAGCATTGTGGTAGATGCATGGAAGGCGGCAATGGTTGCTTCTTGCTGTGGAAGTCTT
CCAAAACTTATCTGCATCGTCTTGTACCGAGCGTCCAGTTCCTCCTTGGTGTCGCGAACT
CCACCTGACAAATCTCTTCTCTGGAGCCGAACGCTTATACTCTGCCAACCAGATAATTAA
GCTCACAGGCGTTCCGCCTTGAGTATTCAAAACCGGAATGACAGTCAGCGCTTCGTGCTC
TTTTGAGGAGCGTAATCCGAGGTGTGATGATCCGAAGGAATCCAGCAAGCAGTCGATACC
CTACTGAAGATTCCATGAGTGACGAACAGGTCAGGAGCACTCACACTTTACTGTAATTCT
CTTAGGGCCTTAGGACACGCTTCGGAATGTAGCACCTCCGTCACAGATATCGTCGATCAC
CAGAAGCGGAAGTTCAGGATCGATATCCGAAGGATCTCAGAATTTCAAAAGCCTTCGATT
TTACCGGTCTGGAGATCGCGCTTCTATTGCAACGAACGAATTACAGCGGCTGCTCCGGGC
CTTTCTTTTGCAAAAAAACTTCGGTCCGCTTTGTGAGCGTTCGATATCAGGGGCTACGAG
TTGATATTGCTCCCACTCTGAACGGCTCAGTAGGCCACCGAAGACCGAAGATTGACGGAC
AACTTCGACGTTGTTAATCAGGGTGCAGAGTTGCGTCGCTGTGAGGGTCTACAACGATAA
CTTCGGTCATATCCGCAGAGTTCAGCATGTTTGCAAACACTTTCAAACTGAAGGCGTCGC
CCGGATTGCACCGACGCGGTCTTGGCGGAGCATACGGGACATAACGCAGAAGAGTCATGC
GGATTAATCATTTTGCTCGATCGCCTCGGTGACGTTGAAAGAGCTATGATATCATCAATT
ATTCTGAATCTGAGCTTTAACGTTTACGAACGCATGGCTTTCGATGCCGCCCACTATTAA
CGCCCACTTCGCCGCCCGGGAACGTCCACATTAATACTTTGCCTCTTGCCACGGACGGCT
CTTCATAACACCTTCCATTTCCCGAAGGCAACCTACAGCATAACTACATCCTCCTCACAG
TTTCACAGAGAAGCGATTCCGCGCATTGCTTCCATCGTGCTTCCGGGAACACCGTGTATT
GTGGACTTATTCCCATGACGATTCTCTACAATCAGAGAGATAACAGTGTAGCCAAACTCT
TCAGCCATGTCAATGTATGTGCTACCTCTCGCCCTGGTACTAGTATTGATACGATAACCG
CATCTAGTCCTATCTCCATAGACCGGGTCGCGCTTTGTCCATTAACTCTTTGTGAGATTC
AACTACCCTCAGTTTTCCAATAATACTTGCGAACTCCATTTGGTAATCGTCCGTTTCGCA
GATAGATTCTGCCGGCCTGACTCCCAAGTTTTGTTGTTATACAGAATTCGCAAGAGAGGA
CTTCCCGATTCGGGAGTCGAACGATATACATGATCTTTTCTGCATACTTCCTCCTTTAAA
TTCGAGCCATATCGAAGTAGGTTCGATCAGAAGTCGTCGCTGCATCGGTCATTTCTGAGA
ACCGAGCCTCATCCACTTCATTTACCAATAAGAAGTTCAACAGAGGATAGAACTCTTCGT
CAAGCGTCTGCTTGAATCCAGCCGGGTCTTGCACATCGCATTGACCCTTTTTTTTACTAC
CTTGCATCCTCATGATAAACTTCTTTTAAGCCGAGGTAGTGAGTTGACTTCAGACTTCAC
TGTCCCTAGCCCTGTTTGTACCCATGTAACCTTTTCACAAGAACCTCAGAAGACCCCTTG
TATTGTTCCTCCCCAGCTACACTCTATGTGCTCGGACGAAGAATCTTTACAGAACTGTGG
TCCGTTACACCTAGGCGTGAGAGTCAAGAATATATTCCTGAAAGCGGCTCCCCCAGTGAA
GATATCGCGGATACCGATCAAATATCTTCTTCTTCCTCGACAATGTGAGGGTCGTCTGGA
GAGCATATCTCGAAGTTATATGAAGCCTGCGATGTAATCATCTGGGGTTATTCGGACACA
TTTGACCAAGGGCAACGTAATCGCCGGTCAATGACCCCGTAGTAATTGAAGATAAGGGAG
CCATTGTGCTTAGTCACGGAGAACAAGCGAATCCATTCACTTTCTCAATCAGCCGCACAC
GGAATTTAGGATGGAGGAAACTCCCCAGCACCATTCTCTCCCAGATTAAACAACACCTTC
TCGAAGCGGGCGTACACCAATCACCGGAGTGCTGGTCAAAATTACGACCCCGTGCTCCAG
AAGGAGCGGATGCTCATTCCACAGGTTCCCCGTAGACCTTCCGGGCATACTTTTATAACG
ACATACCGTTGAAGTGCTTTAACAGTCACCAGCCAGATAGCGATCAGGTTCCGTACCGGT
AATGTTGAGATTACCTTCCATCCTACTCATTTCAATTCGACAATTGGGCAGAATTTAGCC
TTCTTTGCCTTTAGATAATGATACCAGAATCCTTCATCCTTTTACTGGACGATTTACCGA
CATAAGCGAGAGAATCGGTAGAAAACCCACACGCTATAAAGACGCTAGATGATTCCAATC
CATACGAAACCCCACATTGTGCCAGAGTCCCACCTAGAACATATATCCGGTAAGTGGACC
GAGAGCCAGAGAATACAATACAGCCTACAACAGCAGCGAAAGCGCGCAACCGTTAACGCC
AGCCACACGACGAGGAATACCGGTCTTCGCCAGAAATAAGAGCAGAGGGTTACAATTTGC
CAACCGCTTGAGATAGCCGAATAGACCGGGTCTCGAACGTACGATGGAGCCGCATATGCC
ACGAATTTTATCAATTCATGCTTGTTCCTCCGGGAAGATGTTGAAGTTAAGTACGTTATG
CTCTTCTGCTCCATGGAGGCTACCATCTTATAACCTCCGAGCTTGTGAACGATCTCCAGT
ATGTACCAATAGAGAATTTTAATAACATGCTGCTCATCAGATTGAGATTCGGCTCTGATG
GGTTCCACAATCGCCTGCCACTTCCCGTTGAGTCAGCCCAGACCGATGGATGTAGTGTGC
AGCAACAACCATTACAGTTTTCTCTTTGAAATCCTTGACCGGATCATTCATGTTAGTCCT
CCTATTGCTCAGCGTTACTTTTGCCATCGAATACTGAATTGAGCGATCTCAAACTGATCC
GTCCAGCGGGTGTCCCGGAAAAGCCACTCTTCAAAAAAGTCCCGTCTTTGTTTAATATCA
GAAGTCATGAGGGATTATGCGAACTCCGTCGTCATGGATCAAAGTTCCACCAGAGTGTTA
GCTTTAGGTTTAACATCCCCGGTCACGCGAAATCAACACATTGACCATTTTCCCTCCTTA
TTTATGGTGGTCTACAACCACAAACTTGCTTCGCATATATTCCCCTCACTTCGTCGTAAG
AGAATACCTGATGATCTGCGGATTGTCAATACCTACATCAAGCAAGTGCTACCCTTTGAA
GGCAGGCTGCCGTGAGTGTGGCCGCAGGTGCTGCTTCCATGATGGGCAGTATTCCACGTC
TCGAAAGGATAGTGGCACATGACGATCTTAACTCCGTCAATTCGAATCTCCTGTACTCAC
GAATGTCTTCTACGAGAGATTGCTATCGCAGCGATCAACTTCCACAGACGAAAGTCGCAA
TGATTTCCCTTCAGGAAGGTGTGATACGGCTATTCAACTGTTTGATGATATCCAGCACAG
GCTGTAGCTTGTTCTTTCTGCAAATGCGAAGTCTCCCAGATGGTACACTTCGTCAAAAGG
CCTACCTGGGGAGTTCCACCGAGAAATAAGCTCCTCGGTTTGCTCCTCAAAAGTCCACGG
CCTGTTGCAATACTCGATCACATTCTTATGGAAGAAGCGTAGATGACGAAGAGAAAAAAT
ACACATTATGCCTCCATTTCAAATTCGTAATTTTGTTTAACCGTGATGAGTCGCATATGG
CTCCCTCCCAGCAGATGTTCACCCGGCTCCATACTAGCCATAGCCTTGAACTCTGATAAC
ATATCCCGACGCTCGGTGGAAATCTACTACTCCAGTAATGGTCCGCACTAATCTACCTTC
GGTACAGATGCTCCCGACACCCAGCGCCAGCGATCCACTGTATCTTCTTCAGTAGCTCTT
CTGGAGACCACTTCTGTCTACACCCCGCAACTGAAGAAAGAGTAAGGACGAGAATAGTTG
TTGATGGCTTCCTCTACAGCCGACTCAGCCTGCAATTTAATGAACTCCCATCTTGCAAGA
ACTTTTAATCTCGGTGGTGACCGGGGTGTAGCTGTACCGTTCTTTATATTCATTCCACAC
TGTACTGTTCTCCCACATAGCCTTTGAATTTGTCGGAACGAGTTGTCTTCTTTCCGTGCC
TTAAGTTTTCCGTGGAACTGATGCCTCCCCTTCGAGCATGCTCAGTTGGTTGCTATACTT
CTGGATCCGCTGAGTTACCACTATCACAGTATTCTGTAAGACTTGCGATAGTATGCAAAT
TTCACTTCTCTGCAACCGGATGATACGGCAAGCTTCGATATGGTAATTTACGGTAGCTCA
GCGAATTCTTATCGAGAATGACCAAAGCCTTCTACGATGAGTATCACGCCCATGGACTCA
AGTTCTCCCGTCACCCCTGTATCCTCCGTGAACGTCGTGCAGGAAGCAATGCGAGAAGAG
TCCTCATGTACGAAGGTCGTAAGCCTGACCTGGTCCACATCTGATGCTGTACACGCAGAC
TGTATGTAAATCTTTCGATTCAGATGGTAAGTACGGAAGTTATCTCTCCGTACCAAGGAC
CTTCGTGGATTTTGAGCGCATCCTCAATAGCGGCGATACCGCTGAGTGTTCATAGTCTTT
GTAGAATAACGTGTGGTTGCTCATCTTGATTGTGCGCTTGTATTTCCTAGCCCACCGAGG
AAGCTGCCATTAGCCCGTCTCACCTTCCTCTCGTTGAGGTAGTGATCGGCTCCTCTCCTG
TTGGATCGACAGTATACCCATATAGGGTTGTATGCAATATGTTTTCGCTGCCTCCCCACC
ACCCTGTCGTTCTGACACCTTCTGCCGCATACCCCATCGCAGGAATACTGGCACGGAAGC
AGAGCACTCCGGCTACTGCTCGGCCAATTCCGTCGTTTCATTCTGTTCACTATTGGTATG
AGCTACAGCACCTGACCGATCTCCGGTTCTCCCCGGGGCCTCGAAGTATACAGCTTCCAG
CCAACAACCTGGTATTTGTACTACTGGCAGTGCGCTTTGCTTTCTGCGATTTGACCGACT
GGCTTGAATTCCACCACAGGCTCCGTGCCGTATTTTAGACGAGTATTCCATGTCGAGGAA
CATCCCAATGAAGGCTAGAAGGACTATCAAAAAACGAGAGCTTCTAAGGTTTCCATAACA
CCTCCTTAATGTTAGCCCGCCTTGTAAGGGCTGTATATCACTCGGCTATTATACTTCAGC
CCAAGTTGTAACGACGGCGGTCTTTGTTGGGAACTCGACCATCATCGACTGGAATGTGAA
TGCGTTGCCGATCCTTCTGATTTTGCATTTTTCACAATCTTACTAGCCTGCGCTCTTGCC
ATATAGTTTTCCCGGACTTACAACCAGATCGGCTGAATTCTGCACTCGGGATTTCGGCAC
TGTGCCGGGCCCAGAAGCCGTACCAATACCGTCGATGTAGTACATTAAGGTACAGGTTGC
AGACGCACCGCAGTGTTGAAACTCAGTTTACTGATGTGCTTCGCTCCCCGGGTTTCAGTT
TCAAACAACCTGAAGTCATGTAATTGAATTTGTAAGTGAGAGCATCCATGAACTGAATGA
CACTTAAATCTGGATAGCAGAAGTATCCAAGAGCTGGACTTCTCAGCAGATCTTCGATTG
CAGTGGTCACATTGACGCAGACTCGAAGTTGAACATTCTATATCTCCTATTCTTCAGGAC
AAATACACCGCCGATTACCGGGTCAGCGCGAATAGTTTACCGTTGGGAACCGAGCACGCA
AGCGTCTTTTGAAACGACTTTGAATTTGCACACAGTCAGCCACTTTACCCCGGTAGTTCT
CGCAAAATTATGATCTGGTCTTTACGGGCTTCTCAAAGTCGCCATACGTCACCCTCGCTA
TGTTCTCGAAAGGGTCGAACACCACCAGAACAGCGTTTCCATTCTTGTCAACCACGTAGT
TCACGATCTGCATTTGTCGAAGAACTCTTTGTACTTTGAGACCGCTGGAAAGCGCTTACC
AGATTCGTGTACAGTTCTCTATTGCGCCACATGATACCCTCATAACCGTTCTGACGGTAC
ACATCATGAAGACGTTTCAGTTCTTCAAAGGTGGAGCAGAATACGTACTCAACAAAACGA
ATGCCTCCCCCCCAGACCACTGTGTAGTGCAGACTGGAACTGAAGTCTACCAGACGCTCC
TAAATGTTATCTTTGCTATACCAGCATTACGGAACCTCGTCTGCGCCAGCACTGTAAATA
AAGTTGACGTAAGGTCCACTTCAGGAGCATCGATCATACCCTCCTCCGTTCTCTAGAGGG
TAAGCTTTCAAGAGCCGGCCTCATCCACTTATCGTAAGCATCCTCGCTGGACCGCCGCCG
TGAGTTGATATCCTCCAGCACCAATACCGTGACAGTTCGCCGTGTCCCTAATATCATCAC
CCTTGCATGATCTCTTCGAGTTCTTGATGATGTGCGGTACGTTGTGCTTGCCCATACGAG
ACGCTACGTCACCTGTTGCCGACCTTTCTTAGCCATGCATCGCAGACCGGTCCAGCTAGA
GAACCATAGCCTGCTTTATACTGCAGGATCGAGCGGCTTGGCGAGCATAGCCATTACCGG
TGACCGTTGAGTTCTTCCTTGGTTGCACGATAACCCTCAAACTGCTTGCTCACCTTGACT
GAGCCTCAGACACGGCCTCAGTTCTCTACAGAGCTGTTTCTGCTTGCCCGCACGGATGTT
GGTAACCCCGGAGAGGTTTGCCTCCTGATGTTCTTGCCGTATTTCACGACGATATTGAAG
CTACCGGTCTTCCTCCTCTCTCCGGCCACCTTCTACAGTCCACTCCTTGAACCCGCCTTT
CTTGTCGAGTCCATACAGTCGTTGAAAATAACTCATTGATCCTCCTCGTATTTGATGACA
AGAACATCATTCTCCGTAGGTGCCTTCAAGACCGTCGAGCCGATATTCGTGCCATAGTAC
ACAACTACTTTTGATCCCGCAGCCAAACCGCACCGAGATAGCACAGCAAGAGTGAGTGAC
GAACAGGTAGCCCCTTGGGCACCTCAGGGCTGCAAGAGCCCTCAGTGCGTTTCTCGGCAT
GCCTCACACCTCTGCCTGTTAATTCTGCTCTTTCGTCCTCCAGCGCCCGTTTAGGTGCTC
TGGAAGGGCATTATACCCGCAACCGATGATACGGTTGGCGGCGCTTTTACTTAGAACGCA
CCAACCTTGAACGAGTCGAGTCGTATTTCTAGCGAAGGCCTCAGCGGCCTCCATGTTTTA
ATTATGCTTGACCTTTCACGACTAGCTCCACTAGAACCCGCGATCTCCTCGTAGTACCCC
CTCTTCAGGGCACGCGAATTCATGTAGTACTCTCGTCAGTGTCTGTCAAATAGACAACGC
TAGGCGGGTAATAGCTCCAGTCGGGTCTCCGTGTTCAATCTCCGCTACCTTCGTTGCCAT
ACACAGTGAAGAGTTCAGAGCCCTCGATCACATCGTCTATGCGCATTACAGTGGGTTCAG
TCGGTTCAGTCATATCTCCTCCTTAGTTTTCTACTTTACAAACGGCATCGCCCCGGCGAA
GGGAACACTTGCACTGATGGCTTTCCGTCTAATTTGTCCCACGCTTCAGCTTTAACACTG
ACAGTCTTTAGATTGATTGCAACCACCTCGCCTACCAGCATATCCATTCCAGTTCCGCTC
AGGCAGAATGCTACTGCGTCGCCGATCTCGATATCCCTTCCTGCTACGTCCTTCATTTAG
AAATTCCATCTGCACCCTTGATTAAAAAATAACGAAGCGTCAGAGGGATCATCATCGCTG
CCGTAGGCCTTTGCCCGAAACTCCTGCTCGTATTTATTCACTTCCGATGGCTCGCTACTA
CCTTCAAGGGTACTGTATAGTTTGCATGAATTTCCCAAGGTCCGGTCGTATTGTTGCCGA
CAGGTAGCCAAGAAGCGCTACCAGCATATCAATCTCTCTTCGCTTCCAGCTCCAACTTGT
AACCATCGTAATCGTATACTTTCATATTTCCTCCTATCGCTTCTGTCCGCTTACGTCAGT
ACGATTTTAATTCTCCGAATAGTTCTCACCTTTATCATCGTAGACTTTATGAGCCCTTCT
GCTGTTTTATAGCAACCGAGGTAAATTCTCTTACCTTCATACCTGATCCGGGCAACGTAC
CTAAAAGATACCGGATACCCGTTTGTATTACGGTAGATGGGTTTATTCTGCTGCTGCTCC
GGAGTCCGTCGCCCACCGGCAATTAGACTTGTACACCCTTTGTCGAGTCCTTTCTATCGA
GAGCGCCTTTTCTCGGGTCGTTCTCCATGTCCTCATAGAACCCCTCGAATGTCTTCCACT
TAGGATCGATGGTAACATCCTTATAATATTTGTAACTCTTTTGTGCTTTGCCTCGGTGTA
ATATTCTGCACGATCATTGTCCTCCACAAGAATTGTATGTCGGAGTTCCGTACATTCCAT
GGGGAGTGTGATAGTAGTTTAGACTGCATGCACCGCAACTCTTATTATCTCCTACCTTGA
GTGCTCCAGTTCTAATTTTCGTACTGTTCTCGCAGATGCATTCTCCGCATAGCCAGTTTA
AACTCTTGAATTACCTCGCTCCAGAGCCCCGACTACCCCGGAGATATGTGAAATCGCCCC
GTAGTGGTCTTTAACCGCCATCCTACCTCCTTACTTGACGAGTGGTTACGCCGTAAGCTG
GCTTGCTCCTCGCCCGTTGTGTTTCTTCTGCGCTCGGCTGGCATTGACGCAAAATGTCTC
GCCATGGCTCTTTCGTCTATCCACGCTTACCTTTAGCCTCAAGTTGGAGACGTGTGTGGA
CAAAGATGTAGTAGCCAGATGCATCCTGATAAAATCAGCGGCCGCAAAGGGTCCAGCGCC
GAAAACTCTTCGAACGATACCACAGTCACCTTCTTCGCTTTGCAACAGTGTATTGACCAA
ATACAGCCCTTTGTTACGTAGTTTATCTAGCATCTGAGTAAAGAGATCAGCGATTCTCCT
TTCGTGGCCGTAAACGAGTTCGCTCAAAGAGTGATAGGTGTTAATTCCGTAGAAATGCTT
GTTGCACAGAGCGCTCACTGCTTCTTCGTACTGCTCTTCTTCCAGCTGCCACGTCCCACT
AATTGCAGAGAGTATCTTCCCCTGCTCGGCTTAAGTTTCCGACCCACAGTCGTAAGCTTC
CGCCCCTGCAGTTCGCTACATCCTTCCCACACACAAGCTCCCCACACTTCCACTTCAATA
TTTTCAGACATTCTACCTCCTCTTTCCTGTATCTACAGGGAATCTTATATAGACATATTT
CTTTTCCGCCCTGATGTCTTCAAAGACCTTCCGTCTTCTGAAGATTCCTTTAGTGCTCTA
TCCAGCAAAGTACATCCTTTCGAACCCTGATGCTCTTTGATTACCGTCCAGTTGTGCCTC
GAGGGTGATTTCGTTGGCACTGGCTTACGCCGAGTCGTTCATCTGCCCCGTACGCCCTCC
AGAATAAGCTCCCATACTTCGATCATCGGTAAAACCTCCTTCTTCCGGATATTGTGATTA
CCCACTTCTTTTCCAGAGACTGGACACGTAACGTCCACCACACCCGTGTTTACCAGAGGG
TCAACATGGTCCAGTGTTCCTGCATCCGGGAGCAGGAAGTCCCGGTAAGTCGAGATACCT
TTTTACGGATTGTATCCGCATTAGTATTGCCCAAGACACCTATGAAGTGGAGATCAAAGT
CCGCTGCCCAGTCATCCCACTCTTGTCCAAAAGATAACCCAAACGATCTCATACGCCCTC
CTCAGAATTTACATAATACTCTTGACCGTCGATGATCTCGTATTGGAGGCCCCTCTCGCT
CAGTAAAACGCGAAGTCTGATTTTCAACGCCCCGGTGCACACCCTTTTGCAAGTATCGTT
AACACAGTCTCAGTGTTCTAAACGCAACTTGCAATATTACAGAACGAACCCTCCGAACTC
TGCCCACACCGACAGTAGCCTATCTGCCTGCCTCAGACATAAACTCAAGCTGCGGGTCAT
CCGCCGAAGCTTTGAGTCTGCTCATACACCCGTGACCTCCCTTTGCATCCTTTCGAGTAT
CACTTTCCAGCATCACAGAGAGTGCCAGCTCTCTCCAGAGAGTGAATGTAGTTTCCAAGT
AGGTCCTACCCTTGGAGATAATCTTCTGAAGGTACTACTGCTTTTTGCTGTTGTTCGGTG
TCAGTCTTCGTCCCTTCAGTTCCTAATCAATTCAAACATTCCTGTATACCCTCCTTAGTG
TAAAATGCATTGTCCAGCCCGATAATACGTACTCTGAATTTTCCCCATTGCAAGGCGTTT
CTATCAGAAAATTTGAAAGCTCGGGTATCCAGTTCGTCTCCTCCGGATGTAGAGTTCTTT
TCACCGGCTTTGACTCGCTCCATTTTCGCTCTCAGTTAGAAGTCCTGCAGGGAACATGCT
CTGAAATATAACGGTCGCAATTCATATTCTGTTGAATGCTGCGTTTTTTAGGTTGTTTTT
TGTAGTCAGCGGGAACGACTCCGAAAAGATGGGAGTATGGATCATAAAGTATGAATCGAT
CGATCGGACACGGAATCACAAGCAAGTGCAATTGCCGTCACGGTATTGTGATGCACACTC
ACCTTCCAGCCGAGCATGAACCGGGGTCACATTCCCCTGATCGCAATTAATGAGGACGTT
TGCAGTCTCAACAGACCCGCCACAACCATTGATTTGTGATGTGCACCTCGTCATTATGAC
CTTGCGCTTTCGCTATCCACAGCTCTTCAAAGTTAAGGTCTCTAGCTTATTTACGCTATA
GGGAAGCACTGATTGTTATTCTGCCAACAACGCTCTGTGTACAAGAAACAGGCATAACAC
CTCCTTTATTGAAAAATGGGGCCATAGGCCCCTTTGGCAGCTACCTTCGGTAGCAGTCTT
TGGCTGTGGTTTTAACCCAGCATAGCGAAGAACGGTAGAAGCACTTCTCTTTACTCGAGT
TCTGCACTGGATGGCCTCAGCTACTTCAGCGCCTGCACGAGATACATTAATGAACTCTTC
GGTTCGAACGTACTTGTCCAATTCTTCACAAGTGTCTTTGAACTCTACACGCCCAAGCAT
CGCCTTGCCTGCGGCCTTCTGGTGAGTAAGGTTATGAGGTTTTGGCAACCTCCAGCGCAC
GATCTAGAGCGCCTTGATCGATCTCCAGACCGTTTTGAGCTGCCAATAATGGTTGCGGTG
CTTTTACGCTCTGCAGCGTAAGTTCTTGAAGAATGCACAGTTGAGCTTTTGTTAAATTTC
ACCAATTTTCCTGTGCCACGACGGCGGGTTTTAGGTTTTTCCTCTCAGAGAAACTTCCGT
TTCAATCTCTTTCGTATCAGTCTCTCGCCTCTGCTCCTCTGCTCTTCGCCTCCTCGGTGG
TGACCCGGGTCATCTTTACGGTCTTCTTTATGAAGCTCATATTCGCGAATCCTGAGGTAT
CTTCTTTGTCGATCTTCGGAAGCCTGATCGAAAGCGTTCTAATGCCATCGTGAAGACTGA
AAACATCCCATTCTGCATCAATCACCGCTTCCGTCGCTCTTTCAATTTCCGCGTTAAAAA
TGCCGTCTTGGCGGAACATTTGACTTGACAGAGGTTCCGTACCGGTAAATCTTCAATCGA
ACGCATGACTCGAAACCTTCTTCCATGTGAGCGTGAACGGACCGGAGCCACACGATTGGG
TTTTCGTACTCGACGATATTAACAACTTCTTGCATTATTCTTTTACCCTTTGCGGTTATG
TTTACAATACCAATCGTTTTCAGGAATGTCAACCCATTTTGTTCTGGTAAAGGCTTGACA
TAGAGGAAATTCAGGTATTATCACTGATACCAGCGGAGATATGTCTTTGCGCACTTCACC
GGCCCACGCCTAGCGCTCATGAAGAGGAATCCAATAGCGCAACCTGAACTCGAATGCTGT
TAAAGGGTGGAGACGATTTAAAAGCGGAATGATTAGCAAAACTGATGCAGCCTGCTGCCC
TTCACCCGAACTCATCTGCATGAGTAAGAGGAATTGATAGAGCCATACCGTTCTAGGGCA
GATAAATTCGTGCTGGCGAGCCTTGGGACTCTAACTCTTCCTAGCCGAATCTATCTGCAT
TGGGTGTTCTCCTGATACATACACAAATACTCACCACAGATAATCCATCAATCAACCCAT
AAGGAATTTCTATGAGATGGGTGTTGTGATAGCGGTAACCTCGTTTGAGACTCCAGTCAT
CAATAAACATACTGGTAAGGAAGACGATCTATGCCCTTTGCCGTAGGTTTGTTCTCAATC
CCACACTGGATCATGAGTATTTTGGTGGTTGGTATCCAAGCGAAGGAGTTATCGTCTCTC
CCTACCGGGGACTAACATTGTGAGGGTAGAGTGCGCGTACTCTGGACAATAAGGAAATAG
TAGATTACGGCGAATAATTCTTTAAGGGTTTGACAACAGGAGATTTATGCTATAATCGGA
GTATATGGGGCTTGGCTAAAGCTTCAGAGGATTTATGACTCAAGAAGAAACAAGGGCGTA
CCGCCCTTAATAGAATGCCGAGCTAGCCTCGGGGTATCATTCAAACAGACCTGGAGCAGA
CCAGACTTCTAAAGAAGAGCTTTGCAGAGAACATTAGGTTCCTGGCCAAGAGCAATCTGA
CAACCGAGGTTTCTACAAATGAGAATCAAGCTCAAGAAAGAGCTTGATATGTTCGTCGCT
TACTGCCGACATTGCACTCGCGCATTTGCAAAAGCGAAACAGAATCAGGAGATAATGAGA
ATTGAAGTATATCGCTCCCGGAGTAGTTCCTCTCGCGTTTTGATTAATTCGTCGCTTCGT
TCAATGGTAGACGCCAGACTTTGAATCTGGAGATTTATGTTCAATTCCATCAGTGGCTAC
CAAATGTGGTAAAGAACATTCACCCCGACAAGCAATGTGTCTCGCCTCAGGACAGCGAGT
AAGAAATGTCCCTCCAGAATTAAGGGTCGGTGATGTAATTGGCAACCTTACTGGATTCCG
TAAATCCTCGTATTCTCTGCTATTCGAATCGGAGTCGGCCTGCCAAATTATATGGAACTT
GAATCCTAACGCTCGGCCGGAGAGCTATACGCGCTTTAATTTGATGTCTTTAGAGACCGG
TATTCAATTGGTGTTAATGGAGGAAGAGTAAGGCCCTGTATAGTGACAATAAATTGCTCT
CGTATAGTGTAATGGATGCACAACGGTCTTCTAGCTTCGGGTCTAGGTTCAGAGTCCTAC
GAGCTACCCGAGATGGACGGAAGCTAAAGTGGATCAGCATCTGGCTTTTAACCAGACTTA
TAGTGAGTTCGAGTCTCACCCGTCCAACCAAATTTAGTGTGAGTAAATCAGGATAGGTTC
CTGAGGCCCCCCGCTGGCGAGTACGTCTGTTAACGCGGATGTGGATCGAGACCACTGCTC
ACGCCAAAAATCACTGCCAAAATAGCGGATTTCATGTTGACAAACCGAATTTTCATGTTT
TAACAACACTTTACCGCATTGAAGAAATGACGAAGAGCAGTCCTGAGTGGTCGGGAACTA
GTCTAGAAAACTAGGCCGCTGTGATGTATCCAGGAGGGTTCGACCCTACTGCCGCCAAAT
TAAGGCCCCTTGTGGGTGGAATTGGTACACATCAGTCTTAGAAACTGACGCCGAGAGGGA
TTGAGGGTTCGGGTCCTCCAAATACCAAATTAAGAATCTCGTTATTTGGTTAGTTCGAGT
CCCCAAATACCGGTCACCAAGGTCGGCTCCATGGCGGCCAAGTAGTGAGAGAGATAGGGA
CTGACGAAGGTAGCGCTGCGTTTTCAGACCTCCCCGGTGCCATAAAAGCTATCATGAAGG
GTTGACAAACAAGAAATTCATGATAATATAGGTACATAGAGTACCAAATGTAATATAGCC
GGATAGCTCAATTGGTAGAGCACCCTAATCGATAATCGGATGGTTGAAGGTTCAAGTCCC
TTCTCTGGCTACCAGAACTTTACAGCCCATAGCTCAGTCTGACCAGAGCAAGCCCCCGTT
CAGGCAAGGTCGTCGGTTCGAACTTACATGGGTCGCCAACAAGGTCGTTGTAGAGATAGA
TGGTTACTATCACTGTCGCAAACCCGGACGCCATAGGTTGAGGTTCAATTCCTTCCTTCG
ACCACCAAATAGCGAGAGCGTTAAGACTGGCTGGTGCTGTCACTGGCTCGCGCAACCGAA
CACAAGTGGTTCGATTCAATCTACCAAATTGCAATCACCTGAGAACCCTATGGTGGGTTC
AGTGTCGCTAAATTCATCTCGGTAAGGGAAGCGCCTAAGCTGGCACGGTGGGTAATTGAG
TATTGTGAGGTTACTGGCAGCCAGTATAAATCGTCCGGCTCGGGCCCGTTATAGCTCCTA
GATTTGGAATTAATAGATGCTGTGGCCATAGAGGTGATCCAGTATCTCCCGGAGGCCCGG
GTTAACAACCCATGAATTTAGCCCGCGTTGGCCGAGAGGTTTAGGCGGTGGATTGCAAAT
CCGTCTCAATATCGGTTCAAACTGATACGCGGCTCCGTCATATTGGGATGTAGCTCCCAG
TTGGCGGTATATATTGTTAACTTCAGGTCGCAGGTTCGAACCCTGCTCGTCCCGTTAATT
TCAAAAGAGATTCTTTCGCTTTGTGAAGCAGAGTCGAACGCCCGGATACGGGCCTTTATT
ATTATCTCCTCGTAGCCTCAGTCTGGTTAGAGTGTCTGATTTGGAATCAGAAGGTCGAAG
GTTCAAATCCTTCCTTCCGGGTGACCACAATTAGGGAGTACATAGTGTGAAGATCAAATC
GCGTATGTCCAGCGAAGACTGGATATCATCGCAAACGAAACAAATGTCCTTTTGCTGTAC
GCGGCCCAATTACGTAATGGAGTTGGAGGCGTCGTCAGAACTCAAGGAAGCGACTTCATG
GTGTCTCTAGTGTAAAAATAAAAAGCGGGTATAGCTCAGTTGGAGCGGGCCTGCCTTCCA
AGCAGTTCGTCGTCATTCGAGTCCGTCTATCCGCTCCAAACAATGCATTTGAAGCTAACC
AGGTAGAAGCGCTATTGAAATTCCGTGAGGACTTGATCGTTACCAAGCAGATGCACCAAA
TAAAGCAAGCAGCTATAGATTAATTGGTTGAAATTCAGACTTTCAATCTGGTGTTCCGGG
TTCAACTCCCCGGTAACCGCCTTTAATCAAGTCCAAGCCGGAACATTCCTCCGTATGCTT
TCAGCCGGCTTGGCATTATTCGTGCGGAGATTGTGTAGCCTCGGATGCTGCCTTCGGTTG
TGACCCGATTGTTAGTTCGAGTCCCGCCTCCAACCAATGTCGTGCAATGGTCAGTGCTGA
CGCTTTAGCTCTGATCGTGTTTCTCAAAAGTTGAGAATAGCGGGCTCTCGCAGCAGAGCG
ATAGGTGGTTCGATTCCACAACATGACACTCTTTCCTCCTCGCCTGTGCTGCTGAACGGT
TTTCCCTCCTTCCGTCTCACAGGCCTCCTATTCCCCTGCCCTTGCTATGTTTATGGTGAT
GCACCGGCTTTGTAACCCGGCGAATTCTGTTCCGTCAGGAATGGGGCACCACTCCACTAT
CATCAAAGAACCTCGCAAGCTCCCCGATAGGGTTCACACTCTATAAGGTTGTAGCATGGA
AAACACTAAGGTTCCTGACAAAACTTCGACCAATCGCGCTAGCGACAAAGAGGCCAGAGA
GAATTCTCGATCTCAGCCCTCATGTTATTGGTCCAAAATCTTTCAAACAGAAAGAAGTTC
CTCACAGACAGCGATGCAAAGATCACCGTATTCGGTGGTGCTGCTGGTGCTGGTAAGTCT
TATCTCGGTGTAATTGACTTCATGAAATAAGTATCCATGATCCTAACTTCCGAGGTGTAA
TGACTCGTCGTACCACCTCCTCAGATTAGCGCTCCGGCGGTCTGCTTGAAAGCAACAGAC
CTAGCCTGGGCTTCGTTGACCCTAAAGTCGGCGCGGAAAGACAAAGACGGCAAGTTTTGT
GTTCTCTTCTGGTGCAGCGCTTCCTTCGACACTTCGAACAAGAATCTGACAAAGACCTTC
CAAGGTTGGTTGCAAGTTCCCAGTCGACGAAGGTCAGCAGTTCACAGAAGGCATGGTGAC
ATACCTATCTCGTATGCGTAACCTCGAAATGCTCGGTTGTTCCTGCAATACGAAGATACA
TGTAACCCCGATTACAATTCGTTCCCTTCGTAAATGGATTGAATGGTGGCTAGACCCCGA
CACAGGAATCCCCGATCCCTGAGCTGCCTGGCGTAATCCTCTGCTGGTTTGTACGCAAAG
GCGGTAAGATGTTCTGGGAGACACTGCTGAAGAGTGCATTGCCTCATGGTAATCCCAGTC
TAGCGCCTGACGATGAAGATCAGGTGAAGCCCACAGCTTCAAATTCATCGTTATTAACCG
TTTCGATAACCCAATCTTGATGCTCAACGACCCCTGAGTATGTAGCTAACCTTTAAAGCC
CTTCCTAGGTAGAACGAAAAGCTCCCTAGACGAAGCTGGCACGCTAGAGAAGAGGAGCTG
ATACTTCACAAGGGACTGGTGCCGGAATCGTTCCCCACCCGCCTCTATGGAAGCTTAAAA
GGGTACGCGGGGACATCAGCGGTAGCACGAATCCGAAACCAACAGAAACCCCGACTGACA
GCCGGCGTGCTGATGAGCCGTACAAAAGAAGGCTTGTTCACTGTCGAGATGTTGTCAGAG
AGCAGGTTGTTCGGAGGTGTTTCGACCTCATTCTAGAAACAGCTGCCAAAGACGGTCAAG
ACGTTTGAAATTCAAATCCCATGTGACCCGGGGCTGCTGGTGCTTTATGCTGCCCAGCTA
CTCAGGAGACCTAGCGGATCATGGATTCCACGCCAGACCGAAGACAACTAACAAATCGAA
AGCTACTCGCCCCGTTCGTATCTGTAGCTGAAACTAAATCAAAAGTCGCAGGAGATTGCA
ACAAAGATTTCACTTCGCGGATGAAACTTGAGCGATTCGGACGGAAGTGGGCTATCAAAG
ACTGCTGACGCTACATCAGATGCATTTAATGCTCTATCCCTTTGGCAGCTTGATCCCCTG
ATTTCCTACCGCCTGATATATGACTCAACCAACAAATCACCAAACCAATTAATAGGAGGC
GGCGCCGGCCGAGACTGAAAAAGACAGCACCGGTATCCCGCGACTTCAGGCGAGATTGGT
AGTACAGGCCTTAAACAGTCAATGGTACTATTCTGGAAAGAACGCCGACCAGAACTTCGG
TTCCTTAGGGCATGCAGAACATTCCAATGATGGCAGAAGACCCTACCATCAAATCAGCCT
TTGATCTCTTCGAAATGATGATGAGCAGGGTTGACTGGGAAGTTGATCTAGGAGTTGACC
CCCCGACGAGGCTATGAAGGCCCGTGGTAAATTCCTGAAAGAGTGCATGCATGACATGGA
GCACAGTTGGTACCCTTCATCAAAGAGGTTACTAGCTTCTACACCCTACGGATTCTCCGT
ACATGAAATGGTGTTTAAGCTGACGTGAAGGATAACTTTGCATCGAAGCAATAATGACAA
CAAATTCGGTGCATTAAAGTTGCCAACCGCTCTCAGTCTACTATCACCAAATGGTTGTAC
TCTGAAGACGGTAGAAACTTCCTAGGGTGTGAGCAAAGTCTTGCCAATGTTATCAATGGT
GACCGTTACGTCAACTTAGGTAATAAGGACGGAACTGTAGAGATTCCAGCTAAGAAGATG
TTTTTTTTCCGGGTCGATGCTAAACGTGACAAGTCCTTGAAGGTAACAGCCCTCCGTGCG
TGTTTAACGCTTGAGATATCGAGTGGAGATTGAAGAGCAAGTATCCGTTGGCGTAACACG
CGATATGAACGGTATGCCTACGCTCTATCTGCCTCCTCGATATATGTCCGAAGATGCCAC
AGAAGTTGAGAGGGCAGCTCCACGAATACTGGCTTAAGCGAGTAATTGCAACATTCAAAT
GAATGAACAATCTGGACTTATCCTTCCTCGCTAAGCTGACCCAGAAAGCCGACAACCCTG
TTCAAATTCGAACTGACTTCGCAGGGCAGTAAGATGCTATTACAGATTTTCTCATTCGTC
GCTGGGACAACAAAATTCTGCAAGCCTTGTTTGCTGCCGATATGCTGAAGATGGGTCAGG
ACCAAGTTGGTTCTGCTACTCTTCTAGCTGGTTGGGCATCTCAACATCATGGCTATGGCG
ATTGAATCGCGGCTCCGCGAAATCAAAGACGTACCAGATAATATAAGCTCATTCCTACGC
TGTTCGCACTGAACGGTGATTATAGTCCAGACTTCAAGCTCGAAGAGCTGCAATACGGTG
AGCCAGATGAAATCGATCTTGAAGAGTTCTCGAAAGGGAATTCAGAGAATCGGTTCTTGT
CGGTGGTCTTGAACGTGACCGTGAGGTTTAAACAAGATTCGAAAGCTCTCAAGATTAAGC
CTCGACCAGACGACGAGCCTGTTGATTAATATCATGGGTGGTCAGTCTCAAGCAGGTAAA
GCTGGAGTAGGTAATGGCGCATCAACATCCGCAGCCGGTAGAGACAACGCCGCTGCAAAT
AACGCATAAGGAGCATTGGTGAATGCGACTCTTGAAAGTGTTATTGAAGCTCAGTAATGC
TGGGCTCCTGCCACAAGGCGGAAGAAGCCCCGGCTGTAGAGGTCACAAAGTCCCTAGACA
ATGAAAAGGGATGGCTTTGTTTGTCGTTCTTGAGCCCCAAGAAGCGACCCACGACTGATC
TACACGTAACAGACACCTACTCCGAAGAGGAAGTAGAGAAAGCTTGCCATCAACTTCAAT
ACCATTGCAACGTAGCTAATATCTTCCACAAGATTGAAACACAGGAAGCTGAGATTGTCC
AGTCCTTCATCGCTCCATCCGCATTCACACAACTGGACGACGGAGAAGAGAGATCAAGAA
AGGTACATGGCTCCAGTGGTGGCACTTCCCTGAAGGCTCTGAAGTAGCCGATGCACCATG
GGAAGGCGTTAAATCTGGTGAATTCACAGGCGTCTCTATCGGTGCCAGAGCTACAGTCGA
GGATATCGAATGACCGTAAAAAAGCAAAACGTAAACTGACCGATAATCAAGTTGAACACG
AGGTGCTCATGTCGCTCTGGTCAGTAAGCTCCAAGGTGGTGCAGCCAACGGGCGGCACAC
TACTCTAATCACAAAGCTACCAATGGCGTAACTGAAGAGCTTTATCGATAAAGCTGCTGA
AGCTACAGTCACAATGCAAAGTTCTCGAGTTCTCTGAAGTTCTTCGATGTATTATGAAGA
TGCCGAAGTTCTCTGCTGCCATGGATATGGCAGAACGGAGTATCCAGAACTAGATCACAA
GACTTCGACCAGCGGTCGAGTCTATGTCCATTCTAAGTCGGTTTATAAATCGGTTGACCT
GAATCAAGCCTTCCGAAAGTTACTCCCCGAGTTGGCATCCCTTTCCTTAAAAGGGATCAG
GAACTGCTTGAAAAGGCTCTGGAGGTAAATCTCCGAGCAAATCCAAATGAAAATCCATCC
AAGGAGATACCAACATGGAAACTATCCTGAAATCGGCCCACCTAGAAGCTCTAGCTGACG
CTATTGCAGTTGAAAAGCTGCTGGTGGTCGCTGCTGTTGCAGAAGTTCAGAAGCAACCAG
ACGCCGCTAATGTAGAGCCGCCAGCTCTTCGTGAAATTTCAAGGCTGCTGAGGCAGAGAA
AACCGCTGCTATCGCTAAAGCCCGTAAAGAGCAACTGGCTGAAGTCCCTCCCGGCCGACC
GCAGAAGATAACTTTCGCCCTGGAAGTTCGACGATCAAGCTTTTAACAAACAGTGGAACG
CCCGGCATTCTTAAAGCTGCTGCCGACCAAAACGACATGCTTGTCGAGAAAGGTGTATCG
GGCGCTGGGGAAGAAGACGACTTCGTTGTCGCCGGTGTTGCCGCTATCCTGAAAGTTTCC
GCCACTTTCTGCCCAAGTAATATTTCCAGCCGGCCCAGGGAATTCCAAATGGCTCAAACA
TTATCGCAAAAGATCATCAGCGTCTTAGCAACTGGCTGAAAGAAGAACAAATGGGCCACC
GTGGCCTGTTCTACACTCGCGAAACTCTGCCTGTCGCCGACATTGATGTTAAAACAACTG
GTTCTTGTCCCTAGACAGCACTGGCAAGTTGGTAACTAAGGCTACTATCGCCGACGCTAC
CAGCATCCTATTTAATACCGACCTGCACGACTATGCCAACGCTCAGATGAGCCACGCTGT
CGTCCTTGCTCGCGGTTGCCGTAATCGGCTCCAAGGCTGTTATCTTCGGTGCTGACGTTG
ACGACGCTGATAAAGTTACCGTCTTTTAAAGCCAAGAACATCTTCGCCGTTGACCAGATC
GAAGTTGGCGCATTGAGAACGTTACCTTCGCGTAAGCCGAGGGTCGTTCCAGTAAATAAA
TACTAGGAGTAACTTTAAAAAATGGCTAACACCCCGTTCTACCTAAACGATGGTCAGTTC
TACATTGCCGACCAGACTGAAACCTGCTTATCATCCCGAACACTTGGACCCCGTCGAGAA
CATGGGCCGTCTTCACAAGTGAAGGTGTGACTCAGAAACCGTCCAGTTCGAAGAGATCGA
AACTCGCTACGGTCTGGTTAAGGATGCCATCCGTGGAAAACTCGTCACCAAGTTGCTAGC
GACCAACGTCGTCAACTGCGTGCCTTCGCGATTCCGCACTTCAACCACATCTTAATTACA
TCAATTCAGAAGATATTCAAGAAGCGCGCTGGTGCTGACCGCGAAGAAACTCTGAACGAG
GTTCGTGCTCGTAAGTTGAAACCATCCGTCGCAATCCGAACTGGCTAACACCGCTGAAGT
TGCTTCCGTTAGCGCTATTAACAGGTAAGAGCTACGCCCCATCCGAACCATCGAGTACGA
CTGGTACGACCTGATGGTAAAACTCGTAAGGTTGTCGGCTTCGACCTGACCAACCCTACC
ACGTTTGACGTTATGGGCAAGACCGAAGAAATCTTCGTCCATATGCAAGACAACAGCCAA
GACGGTCTGATCCGTGGTGACTCGTTGCCCTGTGCTCCTGGAAGTTCTTTCACCGCCCTC
TGATTAACCATCCGAGCATCAAAGAGTTCTACAAAGCTACCAAGGTTAGTCCTCAATACT
GGCGTGAACGTCTAACCGCTCGCGGTCTGACCTTCGCTTCCGTGAGTTTCTACTTCGAAA
CATTCACTTCATCGAATACCGTGGTGTTGACCCGTACGGTAAATCGTTCTGATCCCGGCT
GGCGATGCTTACTTCATCCCAACCGACAGCGGTGACCTGTTCGCTCGTTACTTCGACCGG
GCAGCACTTTCGATGACCTAGGAACTGTAAAGAACTGTACGCTACCGAGCGTATGGTTGA
AGACCGTCGTTCGATCCTGATCGAAACCGAGTCCAACCATCCACGTTCTGCGTCGTCCAC
AGATATCGTTCGCGGCACTGTGAATGCGTAAGACTTTGTGGGAGGTTTCGGCCTCCCCTT
CGTCGGGGAGGGTACCAAAATGGCTTAATTGGTGATCCAGCAAACAACCCTATCGACCGT
CTACGAGAGATTGTAGGGGACGTGGGAGCCTCCGCTCGATGCTTTCCGACGAAACCTATC
AATGGGTGTTGGACAAGAACGAAGGCAACGAAAGACGAGCCGCACTTTGAACTTATGCGT
ATGATGCTCTTCCGCCTGACAAGCGAGGGGAATGAGAACGTACAGGTGATATCGAAGTAT
ATGGTGCTGAGTATTTCAACAACTACTTCGAAAGCTCTAGCTCATTCTCAAAGACCGAAT
ATTACAATTTCTTTGGCCGTGCCATATGCTGGCGGTATCTCCAAATCGGATAGCTGGCTA
ATGACCTAGACCCCGACAACGTAAACACGGAATTCTACATCGGTTTTGCAAGACGGAGAA
GCTTAACCAGTGCAACCCCGGTCCTCAAGACCTTAGGCATGGGTGTGATTATCTTGGCAA
GCTTCAGTTTTAAAAGATCGCCGTCGCCTCACATCCTTGATTAAGAGGGTGGAGGCTCTA
GACGGAACAACGGTAGAAGTTGGTTTCTTCCCCTCGAGGACCGATATAAACGTCTCTGGA
AATGTTCCACCAGTTCACAAGTTGCAGCTTACAACGAATTCGGTACTACTGCAACCCAAC
CCGGCCATTCATGGCTCCAACCTTGAAGAATTCACAATCTCAATTCCACTACGCAAGACT
AATGAAGTCTACGTTTGAAAACGCTATCCGCGATGGAAGACAGGTTAATACTCCTGAAGC
AAGAATGGTTGCAGAGCAGATGCAAGTTAACATTGACGACTACCCGGCTCTAACAGTCCC
GCGCAGCTTACAAAGGGTTCAACGATCCGTTGTTCCGATACTGGCAAGATTTTGAGTCTG
TCAAGTTCCAACCATCGGAGGCAATAATGCTCTATCCTACCTTTCCATGACCAACTTTGT
CAAGCTAGATTTGATTAGAAGAGGGCAGCCCGGAGACGACGGTTTTGGAAGACCTACCCT
CCAGTTGAGACAGTAGTGACAATCACAGCTAACGTTCAACCTATCGAAAAAATCGACAGA
CACTCGTATCCTTCCAGAGGCCGACAGAAGCAAGGCTTTGCTTCAGGTAATATCTAGAGG
CGAAGAGATTCGCCAGTTAAAGGAAGGACCGGAGGTGGAGCGATAGATTCATGTGGAAGG
AGAGTTGTATTGAAGTTATGAAAGTCATCAACTATTCGATGGGAATCCTAAACCATTACA
AAGCAATTTGTATGAGTAGAAGAGGAATAGTACAGCATGATCCATACAGAGCTAGAAACT
CTCTCTATCTTATGATAAAAGAGTTGTTCCCTGATTGGCGTGTCATCCAAGCATACACAA
ATAATCAGGAACCGCAAAACCCCTTATCTGGCTATCGATATTAAGAAGCTAGATGAATGG
GAAGGGAAAAATGTCTCCAACATGAGCGATCCCATTAGGTCCCTTCGCATGGAACTGTTC
AAGTTCAACAGGACTTCGCAGCCAAGGTCGTATTCGAACTAGTTGGTAAGTATGGAGAGA
CAGCTGTCTCTGACATGGCAATGGCGATTACCGGGCTCTGCGTACTCCAGCAGGACACGC
CGCCCAAAGAAAGTTTAATCTATCCCTTTTCAAACTCCCTAGCACAAGACGAGTTCCTAT
GCTAAGAGAAACAGATATGTACATGTTCTATCAAGTTACATGTGAATTTGGATTCCTCTT
GTGATAGAAACCACCCTAAGAGTTTGTTGTTGGGCTGATATTCACGGCGTTTATTACGAC
GCTGGTCGCCCGGGTCACATTATTGAGTCCCACATCGACATAAACTTCGAACACTAATAG
GGAGACAGGAATGACCAACTGTCTTTACCGATGTAATTGACATTCAGATCAGCCGTGAGA
CTGTTGCTGTCGCACAGACAAACTTCAACGTTCTCTGTTCATCGCTTTCACACAAACTTC
GAACGTGCCAGAGTCTACAACAGCCTGAAAGGTGTCGTTGAAGACTTCGGAGAATCCGGA
TCCAACATATCTTGCCGCCGTTCGCTACTTCGGTCAAGCTTCCTTAAGCCACGCAGTCTA
GTTATCGGACGCAGACAAGTTCCTTCTGCTACCGTCTCTGTCAGTGCTGTTCAGGAAGCT
GACAAAGCTACGTCCCAATACCGTTAACGGACTACCTGTCAGCTATGTTAGCCAGCAAGC
ATCTATCAATTGCTACTGATTACAGGCCTAAAGGCAGCTTATGACGTACCCGCAGCCGGC
GTAACCGTCACAGACAACGAAGACGGAACTCTAACCGTCGCTTCTAACGAAGACTGAGCC
TGAAGGTTTCTTCTTAACCTGACCATATTGCTGCCCTACTGAAGGTTACGGCTGCTATCA
CAGCAGCAGTTCAAGGGAGAAAACGACGAATGGTATGCGTTCTGAGCATTGACAGCCACG
CCGATGATGATATCATGGCAGTAGCTACCATATCGAGGGCAATCTCAAGAAAGTGTTTAT
CGGAGCCACTGCTCAGCTAACACCAAGACTTCCGCAGAGAATGACATTACTTCGTCCCTG
GTTGCTGCCGGGTTCCAAACGTACGGCTCTAATTCATCCAAACGCTGACGCTCACTCCCT
GAGTGCTTTTGGTAGGGTACCAACTGCAAAGAGCAACTGCAGCAAACACATGGACCCATA
AAGCTCTGGCCGCTGTAGACGCCTACCGTCTGACTCCAACCGAGTCTACCAACCTGAAGA
ACAAGAAAGTTACCACGTTCGAGCGTGTTGGTGGTGTCAACCGTACATCTGGTGGGCTAT
GGCCGGTGGTGAATGGATTGACGTAATGATCTTCGTAGATTGGTTGGAAGCCCGCATGAC
CGAGCTGTCTGTGGTCCCGCATGGCCAACCTGAAAATCCCTACGACTCTGTTGGTGCAAC
AATTCTGGAATCGGAAATCAGAGCCCAGCTTAACGAAGGGAATTCGTGTTGGTGGTCTTC
GCCATTGAAGCCCAGCTCCCAGTCTTCGTAATTCGATGTTCTATCCATGTCTCCGAACAT
GAGAGCACAGCGTATCTTCGAATGAGTTTGAAGCCCGTCTAGCAGGCGATTCACTTTGTA
CACATCCGAGGGACCGTGACCGTATAAGTCTGGTCCTTCCCCATAAGGAGACTCATAAAT
GGCTGTTCAACGTCTAGCTACATCTTCCAGCCGATGTTACCATTGTCATCACCACCCTGC
AACTGGTGATATTCCTCGGCGGATTCAGCGAAGACAGTATCGTTAACATTGAAAGAAAAC
GCCGGACACTTTACGTGATGTATACCGAGCCGACAACACATCGACTCGCGTCTGTCCCGG
TTCCAACCACCTAATCTCAACCAGTTCGGTCTTCCATTCAGTATACCGCACCTCCGTTCC
ACCGTTCTTCCTGCCGTTCCAACCCCAAATGCCATCACAACCTGCAAACTCTAATCGGCG
GTAACGCCGCTGAGCCCGGTGATGCGGATACTCTTCGTAACCTAGGTGTCACACTCAGAC
CAGCGCTGGCTGTAATATAGCGCTTATTAATATCGACGGGAGCTGCCTTAACGGGTGGCT
CCCTTGTTGTTTCTGGAGGATAGAAATTGATTACCACCCACTGCGACGTGGTTGTAACTC
TCGCTGGTATCCATTCCGTGACAGGGTACGCAGAGGGAGAGTTTATCAGGAATTGTAAAA
GATAATAGTTTCATCAAGCACGGTTCGATGGATGGTGAAATCGCAAGGGTCTACAACAAA
GACCAAAGGCTGGCGAGTAGAGTTATCAATCATGCAATCCTCGCCGACCAATGATATCCT
TTCGATGCTTTACAACGAATATTGCAACCCAGTCGTACCCCACCGATGATTAAGGACACG
AAGGGTTCAGCTCTGGACAGCATGGGTGGAAGACCTACCTTAGGGTATCTTTCTCTTCTT
GACAACTGCATCCGCACTTTGGATTCTAGGTTCGAGGTCGCCATGCGAAATTATTGGCGG
AAACGTCGATCAAAGTTTGGTCGAGCAAGCTTTCATCCTGTTGGGAAGCTCTCTTCGCCA
GCCCTTACGCAATTCGCTGGAGGCTTCTAATGGCTAACACGGTATTGACATATATAGCCC
ATCTGATGTTAAAATTCGTCCTTGTGGGTATGCTCACTGTAGTTTCTTTCGAAATGTCTT
GGCTTTCGAGACCCACAATAACAATGGTTCGAGGAATCAGAGGGCACCACACCCGTCATT
GAGACCTTTCTTGCGCAGATTAGGAATCGAGGTTCTTCAGACTTCGGTGTTCAATGATGC
ATTATTCTCGCTAGTAGAAAGTTAGCATCGAAGAACACAATCCGCCCGAATCACCCTTGT
CTGTCAAGATTCACGGTAGCAATTCATGATGAGCAAGCACACAACGCTTATGTCAATGGC
CACCCTAGCATAACATTCACCGATGGAACCGGAGAACAGGTCTGACCATTGATGTTCTGA
CTGGACCGATGGCACGGTATGGAAACCAGCAAGTCGGCTTCGACGTGTTTGAACTGTTCA
TGCTCTTAGCTACCCTTGCGATAAATAAGTATTTATGTTCGGAAACCAACGTACCGTCAT
CCACAATGGGACAGAATACACTATCAACCTTTCTGCCAGCAACCTCGCGGTATCAGCGTA
CTGAAGCAGCTAACCAGCTTGTTGGGTCCATCCCCGTTGGGCATCGCTGACCACGACTTC
TCTGGAGCCATGAAAGAACTGTTCGATAACATCGACAAGGTTCGTTGAGAATTTGATCCA
GAATCTGGATGCCGACAGTTTTCAGCTGTCTGATGGCAATCACCTTCGACACCGAATTCG
CTGGCAGTATGACCTGCTGTACGCACTCACAAAGACGTGATCGAGTTCAACTTCGGCTCG
GTTTTTCTAATCGGTTTCGCAGAGGCACCGACGGTATAAAGACTGGGATGGCAGTCCGAG
ACTTCAGTCACTACCGATCGAGTTCAGAGTGATCGGAGGGTTAGAGGCTCAACAGAAGTT
CTCGCAGCATTGGGAGATTTATGTGGCTATCACTCACCCTCTCTCAAGCTAACGACGTAT
GGGAACTGAGTACCAGTCCCTACCACAGAAGATTAAAATAACATCCTGAGGTTATAGACG
TTCACGATACTATCACCAAGTGGCAAAGAACACGCAGAGGCTGATAAGAAATGATTACAA
AAGGATTGCCCAGCTGGGAACTTAATTTACAGTCGATTCCCGGCCTCACTGCGTTTTGAC
AAGAAGCTCGCTGGGGTCGAAGCCCGGCTAAGAGAATTTCCAAGCTGACGAACAAGCGGT
TTGAGTTAAGCTAACTCTAGATACAAAACTCTGAGAGGAGCTTGCCAAGGCAGCCACTCA
AAGCGGTAGTCTTGAAGAACATCGCGGTAGATGCTGCCGCTGTTCGTCTTATCTCTGAGA
AGCTTCAAGAAAGACTGAATGCAACGCCGATCAGGTTGAGTCTGTTAGGCTAGACCTTAG
CGGTATCCGAGATCAGAAAAAACTTCAAAGACGGCTCTCGGCTGCATCTCGTCGATCTAC
CGGTTGAACTGGGCTTGCCTTTCAGGCTTTAGCCACGCTGTATGAATGGAAGAAGCGCAC
AGAGTCGTTTTAAAATTCATCTGAATGTATGACATTAGCCGCTCTAAATTGCCCAGAACG
TACTGTTCATCACCTATGGATGCGTATTAAACGGTCTTGTTGTCGCTACGCCTCAAATTA
GCTGTTCAGACCGTGCCCATTCGTGATCAAGATGTTCTGAGCAAATCAGACGCGAAGTGA
GAATCCGCATTGACCTAGAGTCTTCCATCCGAGGAGGTGGTGGTGGATCGAGAGGTACTT
CCGGCCATATCCGTCAAGGTATGGGAATGGGATTGGTAAAGAGCTAGCCGGTGGGACGCG
GGTTTACTTCGCCCTTGGTGGTGCGTTCGCCATTATGCAGCTTAACCGTAAAACCAAGAA
CTGCTATCATGCCTAGCGATGCTTTCGTCGGAGGCGGAGCAATAAGGTGGCTTTCAAGAA
TCGTTACCCTCCGTGATATTCAGGGTCTCGACGACACGAGCCCTTGGTAGCTCCTTATGT
TAAGACATTGATACGCTGGACAGGCATCGGATTTCAGCAGGAACAAGTTGACAGCATCTT
CCAGTTCATGGCTGAATATGGTCGAGTGATGGCCTTGACGGCGAAGCGATGAAAGGCTCC
TTCCTCGAGCGGTTGAGCAGATGGGCAAAGGGCGATTATGTCCGAAGAATTGAAAGGTCA
GTTGGTCGAAAGATTCCCGCTGCTGTAGCTCTGATGGCCAAGTCTCGCCATCATGACTAT
CAAGGAACTGATGAAAACGATGGAAGACGGAAGCTTAAAAGGATGTCTCCGATTCCATTT
TGCCAGAACTCGAAGAAGCCCAGGAAAAGCGCGCTTTGGATGTTGCCATGTACGCACCGC
AGCTCAGCAAGGTCGATCAGTTCGATGGAACCGCACTATCGAAGCATCTACTGGCGGATT
CGATAGAGGTATGAGTGACTTCTTTAAGATTGCTTCGCCTCAAGTACGAAAGCCTTCCAG
TAACTGCACTTGGTGGCGGGTCTGAAGCCTTATGAGTATTCGTCAACGCTCTGGTCGGTA
TTGAGGGAGCTTGGTTCCCCAGTAAGAACATTGCCGCCAGTTCAACACGACCGGCAATAG
GGCTGACTCTACCACGGCTCAAAGTCCTAGCCTTGCTGACCCCTACAAGGTTGATCTCGC
TATTAGCCAGTGCGTTGGCACTGAAGACTTCCATTGTCTCTTGAAGGCGTATATTCGGCG
TTCGGGGACCCCTTGAACAACAACGCAATGCCTACATTCAACCGAAACCAAATAACGACC
CTGACGGTATTTTCTGTTGTCCCGCTAGCTGAAGCGCTCAAGCACGAAGAGTTCAACAAA
ATGCTGGTCGCTAATGATGAGAGAACTTGCTGCCATCATGGAGTTCTTTCAACAGTGTTG
TAGAGAGATTCGCAATAGCCGGCAAGTACAGGGACGCTGCTAAGATTGGAAGCCGGAGGA
GACAGAAGTAATTATAATGTCGAACATCGACACGATGTCGCGTTCAATCCGGAAGATGTA
AAGACAAGGCCACCTTCATCGGTGACAGGCTAGTAGCTCAGAACATTGACGCGATCCAGA
CAGAGACTTATACCCGTGCAACCAGTCCTTGACTCCAGACCAGTTTGGGTATATGATGCA
ACGTGGTCAAGTTAGAAACGAAATGAGGCGCCCCTCAAGAAGAGTGCGCCATTGACATTA
GTTTCAACCTCACGTTTCTGGTATCATGCTCAGGGTAACGTCATGACAACTGAAGCGCAA
GAAGCGAGTACGAGATCGTTAGTAACGTTCTTGAGGAAGAAATTCAGGACTTCGGCAAGT
TATAAGGAATCAAATGACAATTGCTATCAAAAGAGAGAACGGAGACCTAATCTGGTTCGA
CGTTGTTACAGAGTTCGGCCGTGGCTCTGTGAGTTCCAACCCAATCGAAACGGGAGGAAA
AATCACAGACCACATTACTACAGAAACCCAGTCTTCACTCTTACAGCCGTCAAACTTCAT
CTTGAACAGGCCCGTTATCAACGACAACGAGGCCCAGACATACAAGATCAACAACAACAA
AGAGCTCAGAATACGCAGCCGGTCAGTGTGCCGGCTGTTATCACAACTTAGATTTGATCA
GCCGAATTTTCCCGGAAGTTATTACCAGTTTATTCCTCCTGAGATTCCTGCAACTGTCCT
CCTCAAAAGGGTGATAAAGTCGCTTATGATATTGAGCGCCAACTGATCGACATGCAGCGT
AATGCTGAGGTCTTCAGTTTGCTGGACTCGAGAAGGTATCATCTGGGATCAAATCGAAAG
ATGCATCTTCACCGACTTGTCTTTCACTGAAAACGTTGAGACAGGTAGCAGTCTTCAACC
TGTATGACACCGAGGGGCCGTGACATTCACAGACACCGCTACATTGAAGTTAGGTGAACA
AGGGAAGAAAGACAGCCAAGAAGGAAAGAGAGACATCAAAGATGGTGAAGGGGCTAACAA
CGCTACTGGACAAGACCTCCCACATTCAAGCGTAGCCAGATGAAAGAGGCTCAAATTAGA
ACTCGTTGGCAGGTGATTAATGAGCATGACTTACATCGACACATCTCCTTTGGTACCAAG
ATAGAAGTATAGATACGTGGTAGCTCTGAGGGCATTTCCCGTGTCCTCAATTCTACTGGA
ACAGCCGCTCTAGGCAGTGGCACATGGACATATTCCGACGAGGAGCTTAATCCAATCCTA
ACAGGGTTGGCATTGGTTCCACAATACCCGATAATTGCAGATTACGCCATATACAATATC
AGTTTACTGGTACTTTGTCACGCCAACCTAGAACAGGTAGCACGATGCAAAGCGACATTG
TACCTCAGTTTTGAACTTCTGTATGTTAATGTTGATTTGGAGGAAGATGCAGAGGAGACA
ATACGACAGAGTATATAAATTGACGCTCGGTAATACTGAGTCTGGTCAGGAGTAGAGATT
ACAAACCTTCATCCTGACGGAAGTGTTAACAGAAGGACTTCAGTTCAGGTTCGATATCTC
TAAAAGCTCCGATAACAAAAGAGCGGCAACTCGGCTACTGTTGAGATATACAATCTGTCG
ATTGCAACTCTAAACCTTCTTGAAACCGAATACCTTACTTGCAGACTAGAAGTGGGGTAT
AAAGAAGCTGGAACTTCCGTTGTTCTAGATGGTAATGTTGTCGAAACATCCACAAGAAAG
AGTGGAAACGACTACGTGACTCAGCTTATTCTTGGTGAGGGTTACACTGCTCTAACTGGA
GACTAAGCTGAAGGGCACAGTATCTCCGGGCAAAACTGTAAAGGATGTTATCGAAGAGAT
TCGTCTTCAAATGCCGGAGTGGACAGAGGAGCTTATACAATTTCAATTGCACAACCCGAT
CACGTACGGCTAATAACCCGTGGTTCGGGCTATTTCTAAACAGCGTGTGTGAAGCGTAAC
AATATTGAATGGAACATCAGTGGAACGTTCTAACGTCACAGACGTTAACGGACCAACAAC
GAAGTGTTCAAATGGCTCTCGTAAACACGAGAGACCGGTCTGATCGGATATCCCATTTCT
ACGCCAGTGCCAGCGAACGGCACAGAAGAAAGATAAGCGGGAGACGGAGAGGCGTTCACT
CCAAATGCCTTCTCAATCCTGAGCTAACTCCCGTTCTTGTCCGGTAGAGTCGGATACAGA
TTGTCAGGAACTTTCAGAATCAACAACGTCCGAATCTCGGTGATACAGAGACAACGAATG
GTACACTGAATGTTGGTGTTCCGATTCCATAACCAAGAGGATATTGACCAATGAGGAGAA
CAGGCCTACAAGAGCTTCTGAACCTGCACTCGTCCACGGAAGGCTCGAAACAGTATACTG
CCATACCTTGTGTGGTATTGCGCGTTCTGGACGATTTTAGACGCCTTTCTGTCGATGTTA
GACCAGTGGTCAATGACCTGTACAAAGACGGCACATCAGAAGAGCAACCAGAGATTCTAT
CTGTCCCCGTCATTATGCTGGGAACAGCTAACACCCTTATCAGCTTCCCATTGAATGTCG
GGGATACGGTGTTGTGTACTCTCAAAGAACTATGGACGTTTAAAGGCTCTGCCACTGGAC
AGCCCTCACACCCAAACGATCTTCGTAGTTCAACATGGCGGATGCCATTGCCATTCTCGG
GTTGTTCACACTTCCCTAGGTCGATGAACGACCCTGCAAGACATATTGGCCACATGATAC
CGTAAAAGACCTTACTACTGCCCGCTACCAACCTGATGACGGGACAAGAGGCGGAAGTTA
GATCAAAGCCAATGGAGATATTCTAATCAACAGCCGCAAGACTATAAGCATTAATGCTCA
AAATGTCAACGTCATGTGCTTCACTCACTGTCGATGCAGCCCATACAACATGGAACGGAA
CATTGCACATACAGGTAACTACGTGCAGACTGGTGGAACTTCTACATTCAACGGTATCTC
CATTCCATACCCACAAAACACGGTGGTGTTATGCCGGGCGGTGGTGTTGATTGCTGTCCC
ACAAGCTAAAAGGAGATCATATGGACTTGTTTGTCAACCCCGACACACATGACTTGGTTT
TTATAATGGTGAAGCTCCAGTCACCAACGAATGGTTGATATTGTTGCTCAGCGACTAAAG
ATCAAACTGTACACATTCCTAGGTGAGTGGCTCTTGATGATAGGATCGGCATCCCTTTAC
TTCGAGAGGATTCTTGGTAAGTCTAGGAGCCTTCCCCGCTGCTGACGCGACATCCATCTG
AGATTACATCCCCGGCGTCCTGAGATACACATCTTGGCAGTCGGGGATCGACCCCTCACC
ACGTGAATACCCATGAGTTCACAGCCCAGAACCACAGACAATGAAATCCTTCCCATCACT
TTCAGAATGATAGGAGTTTAATATGGCTGGAATTACAGCAGAAGGTCTAACGATTAAAGA
TTCTGGACCAAATCCTGACAGACTACAGAAACGTAGCCTCAGGTCTTCGCCGACCTGTTT
CAACTGGCACGAAGTTGACACGTCTTATAATCCTGCTCTAGCCGTCTGATCGGCGTAGTT
GCCCATCGACGAAGCTATCTGGGAAGTTATCCAGATGGTGTATAACTCCTCTCAACCCGC
AGCCGCAACTGGCGGCTCTGGACAACCTAGTGTCCTCGGCAATTAGCCGCAGCATGCCGC
CCGTCCGACCCGGCTCAGGTTGTTCTAGAGGGCAACATCGACACCGTTATTAACTCGCCT
CCCGCTAAGCATGCCGATTTCCAGCAGTACAGGCCGAGTGTTCCACCTGCTACAAGGCGT
GATCCTCACTCCTAAAGCTTGCTCTGGTGTTGGAATCTTCCCTCAGACAGTAACGACCTT
CCGACATAATGAGCTTAAGATGTACGTTGATGACATTAACACAACGTCTATCAAGTACAA
TAATAAGCCCTTACCACTGGTAATAGTAACATCCGAGTCCAACTTCTAGCAGGACTGGCC
GCAGACGTAGCTACAAATCACGGAAATACCTTGACAAGCTACGAACAGAATGGCATAATT
TTCATAGTACCCATTGACCCATTCACCAAGACTTTCTGAAGAAGCCCAACCTGAGTATAT
TCAGAAGGTTAGAAAGCTTGGTATCGCTGTTGACGACGTTATTGGCCCGTGGTGCCTCAG
CAGCCTAGCTATCGATACGATCTCCATTCCTATTGCCGGATGGGACAGCGTTATCAACCC
TGTTCCAGCTATCACTGGCCGTCTGCGCGAGACCGACGATGAGCTTCGCGAGATTCCGAA
ACTCTACCCGTACAAGCCACCGATTCCTTGGAGTCCCTAATCGACGGTCTGATGAACGTA
GAAGGGGTAGAGGATGTGCGATTTATTGAAAATACCGACAACCCAGACCCCGTTCATGGA
GTCCTGGACACTCGTTCCCTTCCAATCGTTCTGTGGTATTCCCACAGAGGTTGCACAGTC
CATCTGGCTGAACAAGCCATTCGGGATTGGTTCTGTCGGAGATACAGAGGTTCAGGTAGT
AGACAGTCTAGGCTACACTCAATAGTAACTACCAGCGACCCGTCGAGGTTCACCGAGATC
AAGATCAGTGTAACAAACACAGGATCGATGCCAGAGAACATTGAAGATCCACGGCCAAGT
TGTAGCGTACGGACCGAGAATTACAAGATCGGAGACGACGTAATTTATTCTCGATTCTAT
GCTCCAATCATGGAAGTCCCGGGCTTCCAAGTTAACAGCCTGACGATTGCCAAGAAAGGA
CAGACTCAAGGCATGGCGAACATCGAGATCGGGTTCAAAGAAGTGGCTACATTCGCCGCA
GCAGATATCACCGTAACTACGGTGTAATGATAGAAGGAGAATTCCATGGCTGTCAACCAA
TTTGACAGAGAAGATTATTATCTGAGGTGGCCCGGGAACGGTCACTGAACAGTTTAAAGA
GAAGCCGATTCATCGCTTCCTGCGCGTTGGCTGTCTGGTAAGTTTGATATCCAGAATGCA
CTGAAGACCTCCAGACTCCGGTCTCTGGACACAGCCACCGGAGACATTGTAAGATTAATT
CACGCGGTCTAGTGTACCAAGATATTTTCAACTATTTTGATTCGCTGGAACGGAGCGTGC
AGGTTCTTTCGAAGCCTGTCGGACCCTAACGGTCGGTGCTCCATGGTACTCGGTGTCGGT
GCCCAACTGGTAACGCCAGAGAGCCGAGAGCGATGAAGAGTATCGGATGATCCTGAAAAT
AAAGATCATCAAGAACAGAACAAACTCAACCCCAGAGCAAGCTATCGAAGCCATAAATCT
GTATTCGGTTCCTGAAGTATTCCTAGAGGAGTACGCCCCGCGCCGTCCGTATCGGTATAT
CGGTAAGATTCTAACGAACGTAGAGCGTAGTCTTCTATTCGACCTAGGTAGGTGCAGGTG
CATTGCTCTAAGACTATCGGGGGTTAACTACACATACACTGAGTTCCAAGCTGGCCGGGT
ATTTGCTACAGAAGGCTTCCCCGGGGACAAGGCGTTGGAGACCTAAAACGATCCCACTGT
TGGTGAATTCGACCAACCTAGTGACATAAGAGTTATAAATGGCTGATATCGGTCAACTAC
CTATTGAAAATATTTTCCACAGGCGGGGATATGGTAGCCCCGACCCCGCGCAGCAGCAAG
GCGGATGGGTATTCAGTCTTCCTCGCCAATGGTGGAACTGGAAGTGGAACCTTCACCGAT
AATACTAACCTTAGCGTATTTGCTACCAGAAGGGCATCCCAGAGTGGACTAGCACTCAAG
AGTATATTACTGCAAACAAATCGTTCTGCACAAGAAACGGTTTCGTCTACAAGGCTGTCC
AAAATTCATATTGCGGTAGCGACCCGGCCACCGTTAGTTCTAACTGGGTCCGAGCTTTCG
CAGACTTTTACCACAGCTAGCTCTGCACTTTGGAAGCCTGACTCCGAGAGAAGGAGGGAT
TCCTCTTCATTAGCGCATCTGGTGCAGGTGTTTGACTCTCTACGGCTTATGGATCGGCAT
GCTGAACGTAGCCAACGCAGCGGAGCTAGAGACTACATTTCGGCTCAAGAGAGTTCTGTT
GTCCTGTTCAACCTGGGTTCATTGTGATCTTCAGGGCTGCCAACGCGGTTCCGTACTTCA
ACACCGACACCCGATGGCTACCTTCAACATTACAGCGTTCGAAGAGGCCTAGTCAACGCG
GGTGATGCAACCAACGCAAAGAACCCTTCCTAGGCCTTCCAGCTAACTCTGCTACTATCA
CGGTTGATCCAGCCAACAGAGTTATCACACACACTCTGGTCTATAGAGACGCTGCTGGTA
ACTTCAACGCTGGTGTGATTACAGCCACTCTTTCTGGTAACGCGACTACAGCCAGCAAGC
TCAGGCTCCAGTTACGATTAACGGCGTAGCGTTTCGACGAAGCCAGAACATTGTTCCCGG
CCTAGGATACAAGCTACGCCGGTGTTAGGCGAGGCTCCATTAATGGATTAACATGACCGA
ACAGATAAGACTACCCAGTTGGCTCTTAGAAATAAATTGATACAGATTGGATTAGCTCGG
TGGCCCTGGATGACAGCTCGTTGGTGCTTCACTTAGAGGGCCAGAAGGCTAACGCCACAC
TCGCGGGTTAATCCCCAATGTTGTGTTCCACGCGGAAACCAGTATGGACTAGATTACCAA
ACGCAAGCCAGAGAGCGGCCTTGGTTGACAGATCAATATAAATGCAACCGTCCTGTACAA
CGCAGATAAGAGCAGGTACTTGGTTGCAAAGATAACGGTGTTTCCCGGTATCTGGGACGC
AACGACTAACACCCCTATGGCGTTTTTTCTAGACTACTAGGCCAGTTCTCGACTACGGAA
CAGTCCCTGTTGCACAAATACAGGATTGGCCAACCCGCGACAATTCCGGTGATCCGCCCA
AATCTGACTAATCAGATTGCGAGAGCGGAAACAGGAGATATCGCAGTGCCGCTGCGTTAC
AGCGTTTGGCGCTGTTCTGGGAACAGACCATATGAGTAAGAGCCAGCACTGGTAACGCTC
TCACCTAGTATTCCAGAGGAATGATGGAGCAGAGGTAGGGCTTTATTTGGGGAACTGGCT
GGCCAACTCTCTGAACTTCAGGATCGCAGGCGGTGCGACAGGCATGTCTCAATAACAAAC
TCTGACTGTAGTTGGCAGGGTGAATGCTGCGACACTCACCCGTCTGGCAACGTCAACTCT
GGCAACAGCGTGATTGCTGCCGGGAACGTGCAGGCTGCTGGCCGTGTACCGCGGCGATGC
TTTTATGAGCAGCAACGGCGCAACCTTTACTGGCGGGAGCTGGCTCTGCGGGACTCTTCT
TACCAACTGGTAGACTCTGTACACGCTGCAGGAAAACTGACGTAATGACTGATGCCCAAA
TCTTGGCTTCGCCACTTCAGAGGTAGTGCTCAGGCTGTGGGGAACATATGTCTTGCGATT
GCAGCCAGCGGAAGCGGGGATGTTGGGACCGTGGTAGCTGGTACGTCTCTGTCTGTCTCC
GTCAACTACCACGGAACTACGCTATCCGGTAGCTGCTAGGCATGGGTAGGTTCGGAGCCG
GAGGTTCTAACCAGATTACCTTGTTCCAGAAGGTTGCAGATTATCAAATAGGCCCGTAAG
GGCCTTTATAACTGGAGGTGATTTTAATGGAATTCAGAAACGTTGTAAAATTAAAGCGGG
GTTCGAGGTGAAATAAACCATCCAGAAATTGATGGATTCCTTACGGCGCTGTAGAGGGAT
GCCCTACGTCTCACCCATGTAATGTGAAGATCGCCGGAAAGCGATTGAAGATGGCTCTAT
GGAAGTGGTAGAAAAAGAGACAAATATGATGACTTTGAAGCCAGATTGTGAGAGACAACG
ATAACCCGGGCTGACCGTCAAGTAGAGATTGCTATCGACGATGAAGACGATAAGAGAGAA
GGCTTGGCGTAAGTATCGTTCTGCACTACGTAAATGGCCAGAGCACAAGAACTTCCCATC
CGAAAAGAGCAAACCTAAAGCCCTAAAGAATAAGGCGAGGTAATATATGCCAAACATTAT
GAAGCCTACGGGTATTAACGCTATCTGGTCTGAAACGGCCAGAAGGTTGACCCGGGTGCT
GTAAAAGTTGGGCTTGATGGTGTGACAGAACACTACCTCCCTACCAAACTGCCAACTTCA
TTGAATACAAGCAAGACTTGTTCAACGCGCACGTTAACCAGCACGGTATTCCCGAATGGG
ATAGCGTCACAGAGTATCAGGGTAACCTGAGCTACACTCAAGGCGCTAACGGGATCATCT
ATAAGTGCCTTTAGAACCCATTCGCAAGATTCCAACTGACCCTGAACATTACAGCCGGAT
ATTGGCGGTGGCGTTTGAGGATGCGGGAGAGCTAAGGTTCGTCAACCTTGATCGGGGCAT
GTAACAAACTACAACACCTCCTCTCTGGATTGGCAACGTAGTTATTGCAAGACAAAACCT
AAGTGTGTATAGTAAGCTGAAGGCGACGCCCGGTACGCTATGAAGCACGGGAACGGCTCT
ACTGTGCCAGCGTCGCCACACTGCCAATAATAGCCCAACTCTGACCGCGATCCCGCTGAG
CCAGCTATCGACTTCCGTTCCTCCTGCTACCGAGACTGTAGCCGGGGTTATGGCAGTTGC
TACGACTATCGAGACAGAAGCTGGAGCCACGATACCAAGGCTGTGAGTCCGCTCAAGGCC
GCAACAGGTGTATCTGAAGAAGAAAAGACAACCTTTCCGGTCTGTCTAACGTCACTGCCG
CAAGAGCTAATCTAGACTGCCGACGGCGACTATGCCTTCTTCTACATTCCTGAAGGCAGG
TAGCAATCTGGCAGATGCCTAACAAGGCGCTGGCAAGATCGAATCTTGGAATTACAAGTA
GTGCTACCCAGCCGGAAACCTATTTCTTGAGGAGCGCTCAGAACCCCTAGCAGATGTACC
TAACAAGGCACAAGCTAGAAGTCAACCTAGGCCTCACTGGGATGGTTACAACAGACCCTG
CGGTGATGAAGGCAGACAACCTTGCTGGATTGGCTAACACTGCTACCGCTCGGTCCAACC
TAGGGGGCTAGGCACAGCTTCTACTAGAAATACAGGAGACTTCCTTTGTCGTCTGGGGCT
AACCTGTCCGACCTCACGAACGTCCAAGCAGCTAGGAAACAACCTAGGGTTGAAGGGAGC
GGCTACGCTCGATGTATGGGGTCTTCCTGCGAAATACAACTGCTATGGACTTCAGTCCAG
TCAGTCGGATATTTCCAGAGGATGGGCTCGACTACCAAAACGGCCTTCTGCTACAATGGG
GAACTGGTCCCCGGTCTGTCTGATGACACAAGGACGAATATCCAACTCCCAGCACGAATC
TTGAACGTTCAAAGTGACCGTAATGGGGACGTTTAACAACCCATCGGCCCGCGCTTCATG
ACGGATATGTGGAGCAACACCGGGTTTAGAGTTAGCTGCAACTGGGGCAACTGGTCTTAC
CCGTTCAACTGGTTTGCTATCACTTCCACCCTGTAATTCAGGAGTATCAAAATGGCTCTA
ACTGAGCAAGACTTCCAATCGGCTGCCGATGATCTCGAGTCGATGTTGCCAGTGTAAAGG
CCGTCACTAAAGTAGAGAGTCGGGAGCGGCTTTCTACTTTCTGGCGTCCCTAAGATTCTA
TTCGAAAGGCACTGGATGTTCGCTTCCTCAAAAGCGGTGCTTTGTGGTCATGACCCTGAA
ATAAACGACGCTTCTGCAACCCTAAAGCTGGAGGATACCTCGGCGGACAACAAGCGGAGC
ACGAACGTCTAGATAAAGCAGTCAAATGGATAGAGACTGCGCACTTCAAAGTGCCTCTTG
GGGCCTATTCCAGATTATGGGATTCCATTGGGAGGCACTAGGTTATGCGAGTGTTCAGGC
ATTTGTCAATGCCCAGTACGCCAGCGAAGGATCGCAACTAAACACTTTTGTACGCTTCAT
CAAGATCAATCCTGCAATCCACAAAGCTCTGAAATCCAAGAACTGGGCGGAATTTGCCCG
GAGGTATAACGGGCCTGATTACAAGAAAAATAATTACGATGTTAAGCTAGCAGAAGCCTA
CAAATCCTTCAAGTAAATCTAGGAAGGTTCCTAATGAAGAAGCTATTGAAAAGCTGGAAG
TTCTGGGCTGCTGTAGGCTCTGCAACTTTATGGCTCTGGAGGCCATGGTTCATCTTTGAG
GCCTATCCTTCCTCCGGCGTATTCGCCGCAGTAATACACTGGTAGCTGTCGGTGTAAAAT
TTCGGCAGTGTTTGTAACTGCTGGTAAAAGTGGAGGAAGCGATTTCGGAAACCATAGAAG
ATGGCTCAATAGAAAATGATACTAACTAAACTCAAAACTACGCCCTCGTAGTAGCTGCCG
CAATGGCAGCAGGGTTTGAATATGTCGTTCTCTTTGTACGGCGGCTTAGCTGTTGCGAAA
AGAATGATATCAATCTCTTCAAGCTGCGTCTCGACAGATGGAAGAGCAGTTCAGAAAAAA
GAGCGAGCAGGGTGTGAGATCACCACTGGGAGACTAGAGGCACATATAGCCCAAATGAAG
AGCCGACGACAGGAGCTTCTACAAGGCCACCTGTGTGAGCTAGTCTCTACCAGCAACCCA
AAGGGGCCAAGAAATGCCACAGAGACGATCTCTGGAGGAGCCTTCTACTGGGGATAGTCT
GGACCCTGCTCCTTACAAGCTTGCTCGACAGGCTTTCTGTTCCACAGTACCAAACGATCC
GCTGGTATTGCCCCGGCAAACTGACCGAGGAGGGTTAAAGGTATACCGAGTGGAGCAGAT
ACAGTTAAAGGGGCTCGCTAGAGCATTCTAAATACTAACGGTGCTGCCATGTGCAATAGT
CAACTTCACAAACAATAAAGCTATGGGCAAGAAGAGCAGAGGGCTCTATATGGAAGTAAA
GACGGCAAAGGCCTAATCAAGGTAGCTACTACACGGGGAGTTATCTCTCATAATTGCTAT
CCTAATATGGCTTGGTCTGGATTCTAAGTTCCAGTCTTCCTAAGCAGGAAGAACGTATCT
TTGCGATGCAAAGAGATATGGTATCCGAGGCGAAACTTAACGCACTAGAGTCGAGATTCA
ATAGCGACTGAAAGTGGGGTGGTAACGTAAGAAGAGAACAACAAGAAACCAACCGGTATT
TGATTTTGATTATGAATGCCGTCAGGGGTCGGGATACAGAAACTCAATAACTATAACCCG
TTGACCCACACTGGGAGACAATATAATGACGAAACAAGACTGGCCGCTAGTTTCAAAGCG
GTCGGGGCTTTGTTATCTCCCTCCTTGTCTGTATGGCTATTCTGGCTTTCACAGCGATGA
CTCGAGGCTCGGTTTGACTCTGTACGCCAGACCTACGAGGTCGAATTAGCGAAGATCTGA
GCGGCAGTATGAACTCAAACTGAACAGACTACAGGATCAGATAAACTCGGCCTCTCCGTA
CACACAGGATCCCGGCAATCCCGTTTGCAATATAGAAGAACTCACGACTCGCTTAAAAAG
GAAGATTAGCCGAACTTGAGCTGAGGAGAATAAAGAAACAAGAGTAATTAAGCCCCGGAA
GCGAAGCTTACCGGGGCTGGCCCCCTGACCTGTAGGTTACTTCCACACTTCGTCAAACAG
CTTGTCAATTTTTTTCAGAGATAATCTTGTTTACCTTTTCTTGACGCTCATTATCTTCTG
GATCGGAATGGATAAGCTCGATGTCGGCAGCGTTACCCAGTCCAGCTCCCGTAATGGGGC
CAGCACCTTGACGTTCAGGCAGGTCGCTGATCTGTCAACGCCTGCGACAACCCGGTGGGC
GCGAGTTGTGTTATCGAATGTACTTCCGATGGCCGGTTGGTACAAGTCGCTTACCTCTAA
CTTGCTTCTTAACGTCTTCCAGTTTCCATTTCCTATGCCTCCTCAATCTAGTTTCATTAG
CGTATTGAACTGGTGCCTCAAAGACTTGAAAGCCTGTGGGCCATCGTCCATCCGCTCCTT
CATGGATTATGATGGTGATCAAATTAATATCAGTCCTCAGTCTCCTAGTAACACAGAGCT
AGGAGTCTCTTTTATCAAGCCACAAAGCAGCAAGTGCAGCACCTCCGGCATGGAAGCCTT
GAAGCTGGAATCGTAGCTTCGATAGGCGTTCATACGTGGCTTTGAGGTCGGGATAACTTA
ACTGATGTAATATTGGCATCATCGTCAGCGTATGCTGATCCCCTCTAACTACATGCCAAC
AGCTCATACTACACTTGCCTCTATATCGTTGCACCATTACACCTTCTTGCCGCCTTCTGC
AAGACATTACTTACTCTCGCTTAATGGTCGGAACGGGTCATGGTTGTACTCGATCTTCTC
GGTAATTAGCCTGCAACATCAAACCCCGTACATTCGCATAGTCCATGATGCGAATGATAG
CGTCAGCCAGTTCCACCTCTTCCATCTTCATCTGTGGCAAGTGATCGTCCATCAAATCCT
TACGGACGCCTTCAGCTTCCGCAATTTCAGTGGTAACGAGCATCAGCATATGCAGAGCAT
CACGTTTAATCAGCTCGCCACCAATGACCAGATCGGTGTTCCACTTGCGGTTAACAACCT
CAAGACCAGTCATCATTTTGTTAAAACCGCTAACAAACTCAGACGGACTCTCGTTCGAAT
AGTTCACCGAACGTTGGCTCTTGATGTTCGGCAATTGCGAGGTGTGGAAGGAGCCACTCC
ATCCAGCGTCATAGCACTTTTGATAAGCTCTTGTTCATCAATGTACGAGGAAGACGCCCT
GCTCCTCAAAGCTGACTAATAGCTTCCAGCCCCCCCCCTCGACCCAGACGCGCATTACCA
ATAAGCCTTGGATCGATTCTCAGAGAAGTCTTGTGGAGCTTCTTTCCACGACCTCCATCC
TACCCTACCATTGCCAAAATACACAAGGAGTGGCACATAGCCTTCTCGCTTTCATATCCG
GCGTTTCTATAACTACAACAAGCGCCGGAACTTTCTCCCATCTGGACGTTCAGTTAGTAC
GAAGTCTCCCCAAGTTGATATTCTCCGACTAGCTGGGCGAGAGATTCCTCGATATGGTCT
TTAACTTTGCTCATTAACTTCTTCACTCCTCACAGAACATTTCAAAATGGCAATCACAGC
ATACCAACATGCCGCCGTCATTTACAATTTCTTCGACCGGAGAGATGCTTCCACAGCATT
CACCCAGCCGTTCGACAGCATCTGCACAGGCCCAATAGTCATCTTCACCCTCCTTCCTCC
TCCTTCAGAAGGTACTCGTTGCTAATAACCTTGAAACTAAAGTCTCGGCTCTTATGCTTT
TGAACGCGAAACCTTCCGAACACCGTTCAACGCAGACTCTCCATCTAGCCATTTCAATTA
CATCTGCATGTCTTTTGGAACGCCACGATCCCACATGTACACCGGCACATGTTTAACATC
CTCTTCGCCGTCATCAGCTAGCGCGTTCAGGATTCCCAGAGTGGCCAGTCGCTCTTCTGG
AAGATAGTAACGACTCTCCAGTATTTGTAGATATCGTACACATGGTATTCCGGTTTGTCA
ACTCCCTCGAAGTTGCCTTGAATATTCGGTGCAACCAGTTCACCCTGAAGGGCGATATTT
AGACCAAACTCCGGAGACGTTCGCAAGATTAAGCTCTTCTGCCATCGCGGTGAAGTGATT
GGAGTCTTTCTTAATCTACACGCTTCCATACGACCCTTAAAGAAGTTCTTGACAGCCGTG
AAGGGCCGATCTTCTCGTCTTCTAGACGGAAACCGACATTTCGAGACGCCACGCCTGCTT
CTCCGTCCTTGTGCCAGATAGTGAGAGACGAACCGTCCAGATTTGAATGTCGCTTCAAAC
TTCTCGTCCTTCTGATGAGCCTGATCGTAAGCCATCTGAATATTCTGTACGCGAGCCTGA
TGATCCGTCTTCGGGATGAAGTTGGGGAACCCTAGCTTGGAAGTGTAGTCAGCTGCCCTT
GTTGCCTCCGTTGATGGCTACCTGCTCGGAAGGCTTTACATACTTCAGGATGCCCATTCG
ATCCGGTAACATCTTCGTCTACTTTCGGCTCTGGACTGCCCACGCTGCATGAACAGGTAC
GATCAGTCCTTGGCTAAGCTCTTTGCGAAGCTTCATGGTCTTGATTCGAGAATACTCTTT
TCCACCTTGTTCGACCATATTACGGCCGCAGGAGATTCAAAGATAGGGTTGTCTACAGGG
ACCATAGGTCCACTTCAATGTAGATTGCCAGATCGCCAGCCTTATACTCCGTTTGATTAC
GCACTGCCAACCTCAACAAGCGCCAGATCAGGCTGTCAGCGTTAGGATGGGTCTTCACAT
CGTCGATACGCACCACTCCGGGCTGAGTACGCCGGTCTTGGGAATTGCTCATATTTCCTC
CTTTATTTTTAAAATTTTACTAGATTAATTGTCAAATAGCCGAGATTAAGGGCGTGCTCA
GGCTGCGAAGATTCTCAGCGTCCATGCGGTCGCTTGAATTGACGCATGGCGCCATGTGAT
GCCCTCTTTGTTTCTAGCTGCAATATAGGCCGAGGAACTATCTTCCTTTATTAACGAGCT
TGCAAACTCAGTCTTTCCCCGAACACTATCTCGCTAACTTTTGGGACGGCTCTCGAATAT
CAATCTTCATCGATGCTCTCCTTGTTAAAATTGTTAGATTTGATAGATTACTTGTTAAAA
CCAACCGAGGTCAATAGCTCGTTCTAGGCTCATGTAGCAGATTCTCAGCATCTAGAAGAT
CACGGATCGGCATCAACTCCACTATTCCCCGGTAAGCCATCTGCGATGTACACAGGTACG
TCAGTATACTCGCTCTTTAAACCCGCAGGCACAGGCTACATTGTCTCCGAAGACAAATCT
CGCTGACTTCCCTTGCCTCGTCTCGAAGATATCAATCTTCGTATGTTTTCTCCTGTTGTG
TTGAAGTCGTTGTATTCTGCATGGATTCATCTGCGCCTGTGTCAATAGCCTTGACAGAAA
ATAATCCGTGGTAAAGTGTAATGGAGAGAAATGTCCGTCTCACCGGCCAACGGTATAGCG
TATGAAGGAAAGCTGCAAGCGCATCCTGAGTCGAAGATCTGAGAACAGGGCAGCGACGAC
CCGAAAGCTCAAGAATATAAGGAGCCCATGCTCCCCGCTCACCGACTCATCTGCATATGC
GTATATCTGCATGAGGCGAAGGAGCAGACCAGAGGCATGTCTTAGGCGAATCCGTATGGC
AAGCCTTGGGACGGTCTGCCTCCACTACCGATCTATCTGCATACAAGAAGCTTGACAGTA
TCCTGTAAGCCGTGTAGAGAGTAATCAAATCAACCGCAGAGGTGTATAAATGAAGCATAT
TTTATTCTGCGTGAAGACCTTATCGACCTACTACGTGATCGTATGAAGCGGCTCTAGAAG
CTGGCAGGGGTGGACAACTGGGACTGGTATGAGGCCTCGCTAATCGACTGCGAGATGCTT
CATTGACGATGGCCGGGGCTAGAGTCGAAAGCTCTTGCAGGTGCTTCCCAACCGTAGGTG
CGTGACCGTGGAGAGTCGAAAGCTACCTTCAAAGACCCTTACCCTAGGTTCTTCGGCCTT
CTCGTAGGTATAGCGAAACCAACGACTTCTGCATGGTGGTGAGCTTTGCGTCGTATGGAA
CACCACCTATGAAAAGGCACACCAGCACCTGAAAGTGAAGTGCAAGCGCGGTTACCGGCA
GGGGCTACTCAGAAGCCTTCAGCTACGCAGACACTTGGTGCCCTAAGACCCGGATGCTCT
ACCACCGGGGAGGGATACTAGGATAAGACTCTTAGGCAGTTTGCTGGGGAGAAAACCCAC
GGTGGTTCTGGATCGCGGCATGCTGTTGCAATCGTCGACGGAGTGTTGTACGATCCACAC
TAATGACAAAGAGACGTACCATCTGGCACTGCTGGCAAGTGTAGTAATGGCCGGGGGAGG
CACCGAGAAAATCGAGAGGAAGCCTGATCAATCTCACCTCCTGTGAAAGGCCCCGGACTG
GTATGTCTAAACACAAATCTGAAGTACAAAGAGGAGGATTTATGGAAATTATTTCGAGAG
GGACGCTTCCTCCAAGGGCTTTGTTTACTGCAAAGGTGTCACCATTGCCCTAAGCATTCT
CAGGCTAAGAGATCGGAACTTGGGTATGTTTCGTTCTCATTCTGTCGCCCCGGGAGACTG
GGTATTATTTCGCAGAGTGCCCTGTCTGTCACGAAAGGTGTCTGCAGGTTGCGGCTCCTG
TCATTCCTGTGGCACCGGCCACCACGGGTATAACAAGGAGCGTAAATAATATGGACCAGT
TAGCAGGATTCATGGGCGAACCTTTCGTAATTGCGTTCGACTACGACGAGACGTCAGTCT
CTACCCTCTTGCCTTCGAATCGTCATGGATACCTTCCAGGGGCTTTGGAGGCGCTTTCAA
TGTGATCGTAGTCACTGCACGCCACCCTGACAACAGCGGAGGTTAGAGTGGCTACGGGAC
TACAAGGTTTACGATCTATTTCACGGCACAGAAGGCAAAAGCGACCATTCATGGAAAATC
TTGGAATAAAGTCTCTGTATGGGTTGACGACAACCCGTTCAGCGTGGATAATGATGACCT
TCCCTCCGTGGACAGTGGAGCAATACGGTACTACATGGAGGCAAAAGCAACAGGCTATCA
ACAGAGGAGTAAAGCATGAGCGAGATTATGTGAAGGTTGAAGTGGGAGATCGGGTGGTCC
TTAAAGGCTATTGCGATACATACACTGGATGTGCAAAGCGATGGGCGCAGGCGCATGGAC
GTTTGGATGAACTGCTGGCTACGAGTTCGTAGAGAAGATAGCTGGGTCCACACCGACGAA
CTGGCGGAAGCGAAAGGCACCGTTGTGATGACTGAACTGGGACTGGAAGGAAAGAGTGCT
GTGCGAGTACGTCTGGAACGCCTACACTGACCACAGAACAGTTGTTGACACTCTGATCTT
TACATCGCATGTAAGGCAGCAAAAGCTCCCGTAAGCACCCTAGACAGGGCCTTCCGGTTT
GTGACCACGACTACCTTATGGAATCTGGCTGCTCAGCGACTGAATAAGGTCCGTCCGAGC
AGCTACGAACTGGCCAAGTCGTAGAAGTGGACGTGCCTGTGAGGGTGTAAATCAAGACGA
AACCTCGCCTATCGTAGTGTATGTAGTAGAGAAGATTTACGATAGGAAGGTAGCAATGCA
CCTCATTCTACAGTATGAGGTAGCAGGGGCGCTAATCAGGACGGTGGTGGAGCAGGGAGA
ACTCATTTCTTGGATGAAATGCAAGGTATGTACTTGACAGATTCAACTGGAGGTGAACAA
TGGGTTGTATGACCAGTGGCGTAAGTATATGAGCGTTGTAATCTTAAGAAGAAGAAGATA
ATGAGCGTCAGTGCATGCGGCTATTGAGGCAGTTGGCGAACTCGATCCTATCAAGATTTC
CTAGATTCTTTCTCTATTAAAACTGAAGGAGTTATTGTAGCATGGAGTCAGGTAACGTGA
TGAGGATTGTAGTTGAAGCACGTGGAGAAACAGAGCTACTTCATTTCCACTGGAACGGCT
TTGGGATGTGATGGACACTGCTTGTCTCGAGTTTTATATCAAACCTGGTATGTTCTTTGA
CGGATTATTCGAGAGCAGGCCTGCTTGGAGCTTGATTTCCGAGGCGTTGCCGTTTCCGAT
TCTGGGAAATGAAGACGAAGGCGTGGACGGCAAACTCATGGAGGGGCAAGTTGCAGATTA
TGACCAACTCCTTCGAGTACGTCTTCTTCAAAGGAGGGTTTGAGTGGCTAGACAGACTTA
CGAGTCTGCATCCAGATAGGAGCGGTCGAGGAGGCCGTCCGTTCTGAAACTTGAAGGGGC
GATGGAAGTGTTGGCCCACTATATGGATTATGGGCAGCCTGCCGGAATGGAAAGGTTGTT
AGCTTTGTGGAGCTTAAAAGGCTCGGAATAATGAAATGCAAGAAATATCCCGACTTCTTT
GTGTCCCAGATGAAGTGGCTCCATGGGATCGAAATGCAAAGGGGTGTTTACGACATTCCA
CATTCCACTTGTGTGCCAAGTGGACGGACTATACAGGATATGTCAAAATGACGTAATGGC
GTCAACTACATGAAGCATGGGTGGTAGAAGTTGACCGTGGAGACTGGCAAGACCAAGAAG
TTATGGTCCATATCCGCTGAAAAGGGAACTTCACACGTTTATAGGAGGACAAATGAAGCG
ACTCTGCTACCGGTGTATGCACCTTCGAGGGAGCCCACCATGCACCGCCATAAGACGGGC
ATTTGGTATAAGGGTTGTAGTAAATGTGGATCGGGAGTATTCTATGGCTAAGAAGAACCC
TGTGTTTAAGTTTCTGAAGACACAGTCAACAAAACCCTCGGTAGAGGGATAAAAAAACCA
AATACAGGCGGAAGCCTAAGCATAAACAAGAAGGAGCGATTAAATGAAGAAGGGGTGACA
AAGTGAAGATCAATGATGACGCTGATACCATCTTCCGGGGCCGTGTTGCCACTATCATCG
CAGTAGACGAAGAGGGTTGCGTTATTGGTGTGGGCAGCTACGAAGACGAAAGCGAGGAAG
CTTATCGGAAGTACCTTCACCTGTTCTTCGAGCGTTCTGGAGTTGTCAAGCAGGTGTAGG
AGGGTATAATATGAACGGACCCGAAGTGCAGATGGTGCGGAGAAAACTGAAGCTGAGTTC
GTAGACGTTGGGCTTGGATGGACGCAACAAGTCACTGGAGAATATGTCACCAGTGTGGAG
GTACGAGAACAGTTACTACCAGAACGACGGAAGGCTCACAGAGGTTGAGTTTGCCACCCT
GCGCAAAGGTGGTCCGTTCGAAGATTATGCGAATTTCTCGCCATTCAATCATATGAGGGA
AGGCGACTCGCCATGAAAGGCTACACTGAAACCACAATCTACTGTACATCTACTTCCACA
CGTTCTCTCAGAAATGAATGAGTGGTTGACAGAGAACGCTGTAGATCAATGGACAGTGGA
AACTCTGGGCTCCGGCTATCAGGTGGCTGTTCCTATGGACCGATACTGTACGGGACCAAT
ACGTCTCGGAGAGGTTGACACGAACACAAAGGTGATGCTAAACTCCGTGGTTGTCTGAGT
AAAGGTTTTATGTCCAAAATGACCATTATTTAAAATTGCGGCTATGCACGGTATCGATTT
GTATTCTGACCACAAGACCGCTTGTCCCCAATGCCGGAAGAAAGGCGCTGACAACAGCGG
AGACAACCTAAAAGGTCTATGGTACTGATCGTAATACCGGGGAAGAACACAAAAGGTGCC
TATTGTTGGGCGTGTGGGTACACCTTTGCTCTCTGCTGAAGAGCAGGGTGCGACTGGGGA
GAAGACGAAGCAGGAGTGGAAATTGTGGGAACTGAATTAACCCGCAGAGATCACAACGCT
CAAATTAAAGAGCAATGCTCGTATGATCCGCGTGGTTATCGTGGCTTGTCTAAAGAGACT
TTGCCATTTCGAGTGCTACATGGTTTCGACCCGAGACTGGCGAGATTGCAACTCAGCTAT
ATCCTACTACTACTAATTACGAACTCACCGGTTACAAACAGTGATCATGCCGAAGAACTT
TCAGAGCCTATCGGCGAGACCGGAAGGATTGCGAGCTTTTGGTCAGTTCAGGTTCAAGAA
TAGCAACAGCAAGAAGGTGCTTATTGTAGGCGGTGAGGTTGACCAGCTATCAGCATTCCA
GATGCTCTCAAGGAGTACGCATTAACGCGCAGGTGCGTGCAAGAGTGATTATGAACCGAC
TCCAGTGGTCAGCCCCTACTATCGGTGAGAGTGGGGCATGGAAGCAGCTACAGGCTCAGT
ATGAGTGGCTAGACCGGTTCGAGCAAATCATTCTCTGTTCGACAGCGATGACGCAGGACG
AGAGGCAGCAGACAAGGCAGCCGATAAGCCTGAAGGGCGTTCATGATGGAAATGAACCGT
AAAAGACCCACGAGCATTTGAAAGCTGGGCGTCAATCGAGTTTGTCATGCGATTCTATCG
AGCCCGTACATGGACCCCTGGTATATTGTAGGGTCTGATGCACTCGAACGCTCGAATGTA
TGAAGCTGTGCAGGTTGAGAAGATTCCTCTGCCACCGTTCGCCAAGAACAGAAGGCTATG
GCAGGAGGGCATCCCGTTGGGCCGGATTGTGAATATCGGCGCTGCATCGGGCATGGGTAA
ATCTACTGTAGTGGATGAGTTCACTCTGTTTTGGATTTATAACCGCCCCTCATATGCCGG
GTACTGTGACTCTGAATCGGACTCGGTGAGTACGCAATCAAGCTGCTGAGCCGTAAGGTA
GGTGAAGAAGGCAGACTTGCAATGAGACCGTTGAAGAAAGCGCGGTTCCTTGATCGGCAA
CGACGCTCGGCACGACTTTAGCAATAAGGAAGACGGAAGCCCACGCTTCTATCTGATCGA
TGACCGAGACGGAAGCCCAGCAGCCCTCTCGCTCAAATCGAACAACTGATTATTCAGTTT
GAGTTGCCGTTATTATTCTGGACCCGCTTCAGGATATTCTGGACGGATTGAGCAATGAAG
AGCACCGGTGTTCATGCGCTGGCAGAAGGGTATGACCAAGAGCCTCACCGTGACGTTCAT
CAACATCAACCACGTTCGTAAGAGTGGTGGCGGTCAAAGGCTAACTCGGTGCGGAACTGT
ACGAAGAAGATTTCCAGGGTCTTCGGTCCATCTTCAAATCTGGTGCAGCTAAACATTCTC
CTGATGTGGCGGAACAAGGACGCGGAGAACGAGTTCGAAAAAGAACCTGATTAACGTGAA
GCTCTCTAAGTGCCTGGACTGGCCGCACTGAATGATGGGCAATTGGTACTACGACGTTGA
GACTCATACAATCTGGGACTTGACGACTATCTGACCGATACCCCGACAAGCGCCGTGAAT
ACGAGGCTGATGGCTCAGGAAGGAAGAGTGGTCACGGAATACCAAGGTTGACAGAGGGCG
ATGCTTATGTATCATCCCTGTCGCTTTACAGGAGGAAAGCGAAATGCAACAACGATTTGA
AACAATCGAGCAATACCTCGAATGGATGGAAATAACAATTTCACCAAAACATGACTTGGG
ATATCGAGGCCACAGGCCTGTTGATTTAGAAACGTCTATCGATACACCGCCAGCCCCTAC
AAGATTAAGCCTAGCTACAAACACCACTGCATTGTAGTGGGGAGCACGGTGGCAATATTC
TGGCAGCATTCTACGATAAGGCCGACCTACGTCTTTGACGGCGAGAGTACACTGAGAGAC
TGCCGAAAGCGTCTTCCATGCCCACACTGAAGAAGTTGTGACTCTCAAGGAACACTCCGG
TAGAGTATATTCACCCGCCGCTGAAAGAGAGTTCACAACTATGTGCTCAAGCGCAAGATC
GCTGCTGCAGGCTCACAACATGATTTAACTTCGACCTTTATGTCGATGAAGCTTGTGGAA
GATATGGACTTCACTCATCGGCGTAGAGCACAGGATGGCGGTCTGACTACGTGGTCTGCT
GACACCTGGATGGCAGTTAGCGGGTATTCGACGATACCCTCGTTATCTCCAAGACTTGTA
ACCCTGACCTGTTCGGCGGCCAGTCCGGATAAGCTGTCTCGTCGCAGGTGGTGACACCAA
GATTGACTTCCGTAAGCATTTGGCACCGGATGCTCGATTCCTCGACTCTCGCCGATATGC
CTCTTACCACTCGTATCTATGACGTAAAAGGCGAACACCGCCAGCATGGCATAATACTGG
TTGCAGGACCGACGAAGATTACCAACTGTCCGACCGTGACAACTACATCAAGAATTGGCT
ATTGTCCCCGATTCAGTTGGAGAAGAAGGTTGCAGAGATCATCACCTATCAGGAGCACCG
AGGGTTTGTCTCTGAATGCTGATCTCTGTGAAAAGCTGGTGAAAGACTTTGGACGCCAAG
ATGGAAGAGCGTCGGGCCCGGTAGAGCCTGTGCTTCCTCCTCTCTGACCAGCTACCAAGA
AATTCATGGCAGACTACACCCACCTGTTCGACAGTTCAAGAAGAACGGGAGATCACCACT
TTACCTACAGAAGTTCATTGAAAGATCAAGCTCAACAATTGAAAGAGGGCGATGATCGGA
CTATGATCTTGAGGGTAAGGGAGCATTGCTTTCGCCTCTCCTGGCAGACGTACCTCTGAA
AACGGAGATGCCTGCTAAGATTGGAGATACAACTCCATCGGGCAGTGTATTTATGGCGAG
TTCGGTTGATTCCTCTGAGTGGAAAGAGACGGACCTCACTGTCGACCGAAGAAGATCAAG
CTGAAGCCGATGAAACATCGTTGCTAAGATCGATCGCTGGCTGGATGTAACGTATAGCAC
AGCGTACAAGGAAGAGCGGCTGACGGGTTCAGATTACATGAAGATCACCCTCGTAGCACC
CGCGCGTATGTACGTCAGAGGTTGATTGATCGTGCAAGCGGGGAGCTGGCACTCACCAAC
CCTAGCTTCACAAAGGGCCAAGAGAAAGAAATGTGTCCAGACTCGAACGCATTGCAGAAC
AGTTTCCGTCTGAAGGATGTGATTGAGTACCTGACCTTTAAGCACCGGCGAAGCGCAATC
CTCGGTGGCGGCATGGACTGGGGACGAAATGGAGGATGGAGGAGCCGAAAAGTTACCTTG
TAGCATTCGTCAGACGGCAGGATTCCCTAATGCCTGCTGACACTTGCGGGCTGCTACTAG
CCGGATGAAGCACCGGAAGGTTGCTAACGTTCCTCGGATCACCCTCGTTGTACGGGCTGA
GCTGAGAGTTGTTTGGGTTGGAGATGGGTTCTTCCAATTGATACGACTTCGATTCCTTAG
CACGTAAAGAGTCGGCCTACTGCTACTCCGTAGCACGATCCGACAAAGGAATATTGCAAA
TCTCTTTATGATGGAGAAGCCATTTGACGTTCACACAATGATGGCTGCTGCATTTCTGAG
ATCATTGGTCGCCAATTTAACCGACTTGCGAAAACGTAAAATATGGGTAAGAGATCACCC
ATGTAAACTGTGTGAATTCAGGAAGACTAGAACAGTCAATCCTGACAACGCCAGAGTGAT
CTAGAAAGGTGCAACGATTATCGAAACCACGGCGGAGCCGGAAGGGAGTAGAGTAGGGTC
ATGACCCGAAGCGCCAGGGCGCGAGAGCGTCATGATATAGTCTGGTCTGCATGGGGAACA
TGCAGCGGTAACCTGAGCAAGGTTACGGGGATGATTAACGACCCTCCTGAACACAACGGT
ACATACGAGCTCGCTGGTAAAGTGGCGAAACCATTGGTGCTGACTTGCAAACTGGTCAAC
TGGTGTTTGATGCTTTCGGGATGCTGCATCTCATTGAAAAGCCTGGAAGGACGATTCGAC
CAAACAGTGGTGCCAACGGTAAGAAGTATATTGTTGGAGTGGACGGTCGCCGGGTTCCTA
ACTGCTCGGCTCACGCTATCCTGAACAGCTTGTTCCAGAGTGGCGGTGTTATCTGTGCTA
AACGTGCTATGGTGATTCATGACCAACCGCTGGAGAAAGAAGGTCTGAAGGTTGACTTTT
CAAAGAAGACTGGAAGAACAAGCCGGAATTCGCGCCAACAAATGATCGCGTACCACGATT
GTACCAGCAACTCCGCCAGCTTCCAAGATGTTCCTTGGTCCTTTGTGGTGGCAGCGCCAG
TAATGGCGCGAACAGGAGGAGGCGGAGAAGAAGCCGGAAAGCTCTAGCCTCAGGCTTTAC
ATGACGAACAACTGGCTAGCACTGGCAAGGTGTGGAGTGCGATATCGCTACAGCAAAGGT
GTGAAGGCGGGGTGTTTAGAAGCATTGCCGCGCTGGAGAACTTGCTACTGAAGGCCGTGC
GGAGAGCTGCGGGAGAGTTCACTTACTCCTCTCGAGTCATCGAGCTGACCGCTGGCTACA
TCGTTGGCCTATCTTGGGCCGATTGCCACTAAAAAATCAGTATTGTAAGCGAGAAAGGGC
TTCTGACAGGCCCCTCCGCCTCCTGCCAATGGCCAAGATAGTCTGCCAAAATAGATACAA
GGAGAAACACAATATTCTGGCTTTCATTATTCATCCGGTTAATGACTGTATTCCCCAGCC
TTCGTCGGCTGGTAGCTTTGCCCCTGTTCGTCACCATCATCGCATGGGTTGCAGGAGGGT
TCGTTTACTTTGGCACGGCCGAGAGAATGCGGCAAAGCAAAGCTCACACTTCCAAAAAAT
CACCCACAGCCCTGGATACTGCCGTTCTCCCATCATTGTGAAGTCGGCCATCTGCGTCCT
TTCACAGACCGCATGGTGGATGGTTGGCGGAGTACGCCGCATCAGAGCCTCTATCAGACG
CTACTCAGCAGGTTCTCAGCGAATTCAATTGAACTGGGCGTCAAAGCCTAGCTACGGGCT
AAGCAAAGAAACTCTGAAGGCTGTTGAAGGCAGCAGCGAGTGATACGGTAATCACAGAAT
AGGAGGTATGGTATGAACACGGCTCGACTCTACGTCTACGGCAAATCGTCCATGGTAGGA
ATCAATTGCTGACAACAAGGCCAATCGTATACCATACCTCTAAAGGCTTCTGAGGTATCG
GAGACACCGTTAGCCCACAAAAGCGTGGGCTAACAAATACAAACAGAAAGGAGGTAAATA
ATATGTCTGGTGTTATTCCTGACCATCGTCAAACTCACTCAAGTGGATGACGTCACTGTT
GGTTGGCCAAAGAAGCCTGAGACATGGCGCAGTCGCAGTAGATGGACGATATCGGTCTCC
CCGGAAGGGAAATCCGGTTCTGCTCCCTTCCATCATCCATTGACCGGAGACGTCAATAAT
GGACTTGGAGATTACCAGTCGATGCCAGCGATGCACGACCGCTTCGAAATTGTAGCGGTC
CTGGCAAGAAGTGATATGTTAAGGCTGTTGCCGCACGCCCTGACTTGATTTCCCACTACT
GGTTATTTCGGAACCAGCCCAGCAGTACGGAAGGCTTCGAGTCGTGGTGTTGGTTGAAGA
GCATGCACGAAGGGGAGCCCGTTCTGCATGATTTTTTAAAGGACGCGTTGACATTGGCGG
TGAAACCTCTGACGAGTTGCAGTTCCAGACTCTCTATCCCGAAGTTTGTGCTGGAGGAGT
TCAAAGAAGTGACGAGGCAGAGGTTGTTGAAGCCCGGGTTGACGAAGAGAAGAAAGAAAA
GGTTGCAGCCTGGGCTGTAATGGTTAATGCTGTTCTCGATGCAGACGGAGTCCTCTTAAA
TTCTGCCTCAACTTCGCGAAGAAGCGCGAGCCATGGTGGGGTGGCAAGGCTGAGGAGTCT
CGATTGTAAAGTAATCAAAGGAGGAGCACTTTGAAGACCAACTGGAATGTCCGGTATAAT
CGTCAGTCAAGTGGCTGACACACTCGCCCAGTCTAGGGTACTTAAATAGGAGAAATAAAA
TGGCTTTTAAACTGAACGTAACTACCAACACCAATCCGGCAACGGCGGCATTCGCAAGGA
AGTCGATTGGGATGCTCTGGAACAACCATGTCGTTGAAGCTGCCGGCACTGCCGCTAAGG
CGCGTTCGATCCCGGTTATATCTCCGGTATCATCGATCTCGGCGAGCAAGAGCAGAACGA
TGCAGAGGTTGTGTTCACTGGCACTGCCGAAGATGAAACTAAATCATCAAGGAATATCCG
AACACTACTTCGCTGGGATGGACTGGACAAGCAGACTCGCAAAAGCCTGTACGTCTGAAG
TGTTACCGCAGCGCGATATGCAACAAATCGCGATCACTGTGGACTTCCTCAGATCGAGGT
AGACAAGAGCCTGTACCTGACCGGTAACAGCAACAAAGCTCCGCTTCGTGTCCCAAAATA
ACTATGCGACTTTACTCTGCCGGGTCAGAAGACTAAGATTGTTGGTCAGTCCGTTTACCA
AAGGAAGTAGAAGCACCTGAACGGCAAATGGGCGTTCGCTAAGAACCCATGCTGCACAAA
CTGGTTAGCTACTGGCGTCATCAACGAAGACGAACTCTTTCACCAAGGACCGCGTAGGCG
GCTTAAAGCGGCTCAGTTCCAAGTTCGAGTGTGGATGAAGCCGGGCAAAAAACGACATGA
AATTCCTCACCGGGATATCATCTGGTCGGTATGGTGCCTGAAGGTGTTCCGGTCCCTGAA
CTGGATGAGAAGTACATCTACGGCGTGAACCTGTATGGTGAGAACGACGAAGAGTAAGTA
GCTGCTAACTGGTGTACAACGTCATCATCAAGCGAGCCAAGAACTACGAAGGTTCTGATA
TCAAGGCGATGACGAAGCTGTTGAAGGCACGCGGTGGTAACCGCTCCAGTGGCGACAGCG
AAGAGCAAGCTGAAGATAAGCCGCGCAGACCCAAGCCAACCTAAAGTGGAGCCGGTTCAA
ACCAACGTAGATGATGACATTCCGTTCTAAAAGACGGTGGTGTAAGACAAGGGGCTTCAA
CCTTTTCAAAATTCAAAGGAGGCAACACATGCAAATCAATTTTTTTGGTCCCCATCTTTC
CTTCTGGCTGCTATCGTTCTGTATCGAAATCTGCCGGAGTTCTCGCTAATCTTCTGACTG
TGGCAATCTCATGCCCATCTGGATTCCTCTTGTAATTGTATTGTCGATTCTCGCAATTGT
TTCTTGTAATTGTGGTCTGATTTACGCCATTCGGTGACGTAGTAAACGAACTTCTAGCTT
CTTAAAAAAGCACTTCCGGAATAAGTGAGGACTAATGGATGAGTAGTTTGTTATTGTTCG
AGTACCTGAGTAAGACATTCTGGGTAAAAGGTCCAGTAAACAACCAGTTCAGCGGGTGAC
CTGTGGGAGCGAAATCTCTTCAAAGATTTGAAGAACGCTGCGGAGTGTATCATTAAAAAA
GGAGAGCCGGTGGCTTATGCTGCTCTTCCTAACAAGGTAAACCCCTGACACTCTACAAAC
AGGAAGCGTGGGACATTCAGGTTGTACAGTTCTAACCTGTTGGGGAGCTTCGCCCCTTTT
CATTGGAGATAAAATGAGTGAAAAGGAAGAGGATATTATCGCCTATGACCTATTTCGCCT
TTGCGTGCAAGTGTCAACGAGACCCGCTCCATCATTGTCAGAAACAAATTGACGGGAGAG
AAGAGGAAACTTTGGAAGAATCGGACGACTTGGCGGGAGAATAACAAAAGACGAAGAGGG
ATTCAACGCGAAGACAACTTCGAAATTGAGGACCATCAAGACCCCAAGCACGTGTCCTAC
GGTATCTCTGTTGTTAAAGACCATGATATCGACCGAATCTGCCGCCAAGCTGGGTGCAAG
CAGTTCAAAGAATTCTCCTGTCCGGCCTGACAACTTCCAAGGGACTCCATTCCCTCCCGA
AAGAGTACGAGGAGATTAAGGGTAAAAAAGACCTTCACTCGTGGCGGCAGGTACAAGGGT
GGTAAGCGCGGGCAGTGATTTCATGCTCTTCAGCTAGGTCAGCTAAGGCAGTATATGGAT
CGAAGCGTATGACACCATCATCCATCCCGGAGAAGCGGACGACTTGGATGGCCGAAATGA
TAAAAGCAAAGAATGCGTAAGCTACCCAGAGGGGAGACTACTCAGCGCGTGATCGGGGCT
ACCATCGATAAAGACGCAGACGGCACCTAGGGGATGGCTTTGTAACTACGAACGTGAGCC
TGTTCAAGTCAAGTTCATTTCAGGCCTAGGGATCGCTCTACAGACCTCTATAAGGGCAAG
GTGAGAGGCGAGGGACGTAAGTTCTTCTACTTCCAGAGTTGCCAAGTTCGGTTTAATCCT
GTTGACTGCTACAGACCCAAGCCGACCTGTGCGTAGGAAAGACTTTGGAGAAGTGGCGGC
ATGGCCAACGTCATCATCCCTGCACAACGGACAAGGAGTGCTGGCAAGCAATCTACGACA
CCTAAATCGTGGTATCCTTGAGCCTGTCACTACACAGCATGGGGACGACACAGAGCACAC
CAAAGATGCAATCGATCATGCAAATGTATTGTGACTGTGCTCACATGCAACGCTGGGCAG
GAGACAGAGCGTGGACTGTAGGGCGGTGATGGGGAACCTGGGGTGTAGACCTCTTGTGGG
GACGGCAGAGAATGAGCGTAAGGCCTCGCCCCTTGGGAAGACTTCCCCGGCATTCTGGGA
GACTGAAGCTGCGTTCTTGAAAGCACAGAGGAGGCATTCGCCGCTATCTCTGGTCTAAGA
AACCCAGTCAAGCTTGAGTTTGCTAAACTCGAAGGATTAGATGTAACTAACCCCAGCGAA
AAGAGCAAGCTCAGGAATCCCACTGTATGGGGCTATGTGAGCAGTGTGGAAAGCGAAACC
TCAAGCTAACGTCGAGATTGGACCACAAGACAGGGGAATTCTCCCTGAAGCGAGGGAGGA
CATCCAATCGTTCGTGGAAGGTGTCAGTTTGTCCGCATGAGGATTTTCAGTGCTGTGCAA
AACCGCTATGAGATCAAGACATGCCGAGCGCTACGGGCTGGACCCCGAGGAAGCAGAGAT
ATCGTAAAAGCAGGCTATTGCAATCCAGAAGCAAAAGGGATATGATAAGGTCTTTGCAGG
AAGTGGGAATCAAACCCGCATCCGCAGAGCTTTAGAAAAGCATGGTGATCATCGAAGCTC
ATCTTAAATCAAAGAAGGAGGGTATATGAGAGAGAAGTTTCTAAAATCATGCGCAAGCTG
CCCATCAGGTGGCCTTTGTCTTCTGGCTTGATTACCTGTTCTTCTTGGCTCCGGTTGTAG
CAGCATTAATTGCCGGAGTTAGACTTTTGTACCGCAAAGGAGCAGCTTGGAAGAGGGCAA
TTTGTTCTGTTCACGCCACTCAGGGCGCGATTCAAGCTGCATTAAAACGGGGAAGGAGCT
GGAGTTATGACCAATCTAGCTACATATGTTATCCGCCACAAAAGACTAAAGAGCTTTGAC
TAATTCTAGCGGGGAAACGTCAATGGACCACGAAAAGGAGGAGCGGCCCAGTTTGAGGAA
CGCATATGATAGTACCACACTGGGAGCGTACTATTAAAAAAAAAGAGATATGGAAAGTGG
CCCGACCTGAACATCTTTGATTGTAGAGAGACGGGCATCCATCGTCTGGCTAGCTGGGAA
GCAGCATCTTTTAAAAATCAATCTGTTTTGAGATTGTTGATATTGCTGAATCCTTGAAGC
ACGAAAGCCAGTTGGCGCTCGACCTACTGAAAGATGTTATTAGTTCGAAGTGCCAGATCA
CCCGGAGACGGATACGTCCGACTCACCATTAAAACGTCCACATACGACGACATCAAACTA
CTGAAGGAGGCAAAATGAGCATGACTGACCCCTCGACAGTATTTCATCAAACAGAGCTGG
AGGAAGCAACGAAGCTTCTAGAAAGAGCCCTCAACCTTCTAGATAATGTCCATTGCTACG
ATACAGATGTATGAGGACATTGCAGCAATTCTCTACGGTCAAGCATGAGGAATAATAAGG
TGCAGCGATATTAGTAAACTAGCAGAACTTCGGGACTTGATCGAAGCTGTTCTCTATTCG
ACGAGGCTCCTTGGTTATGATGCAGAAAAACGGACTATATGGATACGGACTTGTACAAAC
GTATCGATGCATTTTGTATTATCAACCAAGGAGGCGAGTGGAGGAAGTAGAATGAGCAGC
AAGAAAACACTTTGTAATCTCTGACAATGCAAAACCGGGTATCAGTCTGGATTACATGGA
AGCCATCGAAAATATAAGTCAGGAAGTAAAACGTCCCGATGTTGTGATTCACATCGGGGA
CCATTTTTGACCTAGCTACCTGTCATCCCACGATAAGGTAGCGCCCTCTGAGGGCCGTAG
GATAGCCGAAGACGTTCAAGTCGGACGTGAGGGTCTGATCGGTTGTTTAAGCCTGTGGGA
GCTACAGGAGCGTCAATCGGGCTAATCGGAAGAAGGTGTACCAACCTCGGTTGATTTTCA
CACTGGGCAACCACGAAGAAAGCGGGCTGATCGAGCAGCGAACGATAACCCCATATTGTG
TATGGCACCGTGGGTAGCGATGAGCTAGGGATCTGTGGACTACGGTTTTGGCGGTTGGTC
CCGTTCCTCGCTTCATTAAAGTAGATGGCATCTACTACGTCCACCATCTTCAAAACCACT
CTCCCCACTGGCAAGCCTCTAGGTGGTACAGCGAACACGCTCAAGGCTGCTGGTCATTCG
TTTCCAGTCATGGGCCACCGTCAGCTTCTTGACTGGGCAATCCATCCGACAATCGACGGT
AAACAAAGGCTTAAGACAGATTGAGCGTGCTATCCTCACGACGAGGCTTACAAAGGCTAT
CAGGGCAACAACAATCACTTCCAAGAAAGCCATGATTCACGAAGTGAAGGATGGTTTTGG
CCCGCTGACGCCAGTGAGTCTAGACTTTATGATGGAGAAAGGAGGTTTCAAACGACAGTT
TATAAGTTCTGGTGAATGGGACATCGGTATCAATGAATGTTCGTGGCGAGACCACCACGA
TGGAAGAAGATGTAGCTAAAGCCTTGTCAGACTGGTGGATTGAGGATACCATTGAGGAAC
TAGGAGGGCGCAGGCCTGCTCGGCTCGACTCTGTTAAAGTGATTGGTTAATAGGATGGGC
ATATTGAGTTTTTCCTTTGGGCGGCCCCTTGCAGGCCTTTGTTCTTTATGTTTAGTATCA
CTCGCGAGCTTTACCGCCGAAGATATCGAGAAAGCACTTCAACATGCGAGAATAACGAAG
GCGTCAAGCATTCTTTCGAGTTGACTTTCGGAGACGGTGCGCGACACTGCAACAGCGGCG
CTGATAAAATCGACCGTAAATCTAAATAATCGAAAAGGAGATATAAAATGACCTATTTCT
GGGCCCACCCCTGAGCAAATGGAAGGCGAAATGGGTATGTGAACCCGGAGCAGCGTACTC
TGGTAGAGAAAGCCTGCACCGACCGTAAATCTTGAAGATTCCATCGCACCGAGGACGGTG
AACTGGACTATGGCATGCTTGCACGACAATATCTGCACCAGCTTGGGTGATCTGCCTCTG
GTAAATGTGGCCCTGACTTCCTGAAGCTGATGGATTTTGTTGCTGCTATGCTGGAGACCA
AAGACGATCTCCCGGATTCCCCGCACATGAACCTGTCCTTCGGCCCTGGACTGCACTGCA
ATCGATTAGCGACGAGAAGTACCAAGTGGCCGAAGAGATTGTAAAAGAAGACCCGACCAT
GACGATTCATGAGTTGACTCAGCAGGGCTAGATACCCCATGGCCTTCCTAGAGTCAGTTT
GGGTATCCATAATTGAAGGGATATGTCACTATGAGAGCGATGTGCCAAGATCGGCAGCCC
GGACGGGTTTCAAGAGTTGAAAGATATGGAGGAACTGGCAACGATATCATCGACGTTCAT
GCTGGGCAACCGATTTCTGGTAACGGTCGCAACCTTTATCATTATCAATAATATTGGGAC
GCTGATACCATCCCTCACGGCACAGTTCTCGGCTTGTTACTGAGAAAAGTGCGATATCCA
AAGGAGAGTTGGTCGTAAGGCTGGCGAGTGGTGCAGATGACGAGCACTCTGTTAAGGTAT
GTAGATTCTCTGATTTCTTCCTCCTGGGAGAGGTTATAACCCAGCCAGTCTCGGCGACGT
TGAACAAGCGTATGATGTTCTCTGTTCGAAGACCCTCCGGCGACGGCCCTTTGGTGAATC
TGGATTGGCTCTCCTTTAATAAACAGTGAGCGCTACTAATGGACTTTTCTGATTGCTGGC
GACCCCGCGCGATGCCACTATTCAACATGGCTGTTCTTAAAATGTTCTGTCGCCTTTACA
CTGCCGGACGATGAGGAACTTTCAGCTATGTCTGGGAATCCTTGAGGCAGGAGCACAAAG
AAGTTATCGAATCCATGCAGTGACGAGGCTTTAGCCAGAGGCTGACAGGACTCCACCGAA
GAGCTTGTTGCACTGGGTAAGAAGGTTGTGTTCAACCCTAGAAGATTACCTAGGCAAAGA
AAACTATGCCACATTGCTACGGCCAACCACAAGGAGGCTGTAGGTAGCGGAGGCTCCTAG
TTACTACACTGTCAAGGTGAACAACCCCACTACGAAGGCTAACATGCCGTATTTTGCAGA
GTGCAACGATATCATCGAAGGCTGATATGCGATTCAGGCAACATGTTCAAAGCGTTGTGG
TAATAGTGGCAGCACAGAACCCAGTATTGAAGGCCGGTAACGATGCCATCCGGGACCACG
AGAAGTCATCTCTTCGCTCAGTTGGAGATCGATAGATTTCAGAGAAAGGGGTTGACATTC
TCCCGAGGCTTGCGACAATAGGCCCGTTGAAACTAGCTGAGGTGTAGATATGATACGGCG
AAAGAAACAGAAGTTCTTTACGGTGCATCCTATCCTCGCTAGCGAGATCGTCTGGCCTCA
CCTCGCAATGCTTCAGTAAAACAAGGGAGCCGGCCCCTTATTGTATCAAAGATTTAATAA
GGAGAGCTTGGCAATTTTAAAAGTGATTCAACACAGTTCTCGGATCGGCTCTTAGGTACG
AAGAATCACAGATTCATCACTACATTCGAACTGGAGTATCCCCGGTTTATTCACAGTGAA
CTTATGACTCACCGACTTTAGTCGAAATGTTGCAGTATCGTGCCATCTCTGCTGCCCATA
TGATCCGACAGGTGGAAGAGAATCCCGCGATGCCCGTCCACCGGAATGAATCAACCGCGT
AAGCTGAGTTCGAACTGATGGCAGTGTTTGCAGCGTAGCTCAAAGTACCGTGAAGATACG
CTTCGCAAAGAGTGCGGCTCTGATCGCTGGGCGCAATTCCAGTAGACTCCAATAAGCAGG
TTAACCGTCTCCTTTGATTTCTTCGGCCTCCAGATGATGAAGACCGTTGTTACAGCCACG
GAACTAATAGATAACTTTCTTCTACCTGCGTTGCTACAAAGACGCACAAACTGGAGATCA
AGGTTCCCGCTGAGAAAATGTATGAGGTTCTTATTAAAGCGGGCCTCATAGAGGCTCTAT
ATGACGGAGATACGTTCCTTATAAAATCGGTTCCGCATTAGGATGGTAAGCTGCGTTTAC
GCAGGATTGGTAACGGGCAAGAACTGAGCGTAGAGGATGCTTATTACAGGTCTTGCTAGC
TGCTGTGCTCAGGTTAGTTAGCAGGAAGAACGACGACTCTGGAGAAAGTCTTTTAATCTA
CAAAACCGATTGGTATTATCAGCTCGTCTACCGGGTTCGAACAGGCAACAATCACGGATG
CTCCGCATCTCGACAAATCAAACGGATGCGGGAGATGGTGACGCACTTGAATCAATATGG
TGACCTTCTGGTCTGGTAACTTCCGAGGTTTCCATCCAACATCGTCAACTAATTCAGCGG
CACGTATAATGATACTACGGCCAAGAAGAGGAAGGAGTTTAAAATGGCAGAATATATCCT
GAGTCTGGAAGACCTCATGCTGGTCGATGGCTTCAAAGAGGCATTCCAGGAGTAATAATG
AGAAGGTAGTGAGGGAGCATCTAGACCAACGGCTGGATGTTAAACCACTCGTATGAAATG
GTTTTCTGTCAACATCGCACACTGATTGGCCGGATCGTAGAGGGTCTGGCTTGATCTCTG
GCTTTGAGCGGACAGATAGCAAGTGGTTGGAGCCTCGGTTGCGCCCCTCTGGAAGCCTTC
CATATTGCAGCGGTGATGATAGTAATCTACGGTTCACTCTTCAAGATGACCAGAGGGATC
GACAGAGGCAACGTTCCACAAATTAATGCAGCAGGAGTAGATATGGAAGCTGCCAGCGCT
CTCGCAGATGAGGCCGATATTTTCAAATAAGATCATTAAAATCAGGACTCCAATCGGAGT
CCTATGCCCGAAATATCCGTCATTGTCGAACTAGCAAAACCGTTAAACTCAGGAAGAGAA
GCGGTGGCCCCAGCGAAATGAAGGTGAGCTGGATAAGTTGAACCTGAAGTTTGAACCTCA
TCAGCTTCACGCTGTACATCGTCCTTCAACTGTTCCTCAAATACGAACTGATCGTAGGGA
GGAGTTTTACGGGTAGTGAAGTCCGAACGTCTCGCCCAGAGGTTAAACTAACGGCTTCGA
TCCTGCATGATGGAATTGGCAGTCCACGCTGAATCTTTACGCAACCAGATCAACGTAGTG
CTGGATGGATACAGATGTGAGGACTCATGTAGCTTACAAGGATTAACCGAGCTTATGGGT
CGAATACCGCCTGAGCCCTGTCCCCAGGCGATGAAGACGATGTTCTTCGTAATTATCTTC
AGCCTAACAGAGACTGCCCTTCTGTTCAGTTCTTTCACATCCGAAGACTTCCAGTGCAAC
GGGTATAACGATATCCCTGTGGTCATCGCACGCGGGCTAACCAGAGTGCTGTGGATGAAG
ACTTGCACGGTGTAGTTTCGCTGGACTATCAACCACATTTCACGCAGAGATTGGGAGCAT
TGTCTGAAGATACTCGGCGTGTCGAGAAGATTTCTCCGACAAGCTATCGACCATGTATAT
GCTCATGAATGTCGTATTGTCGATATGGCGATCCCGGGCGGAGAACTTAATGGGGAGTCG
GCTGAAAACTATAAAGCGTTTGTTCGTTATCGTTTGAATGTCTGGTGCCGAAGACTCGGC
CTAGAAGATCCACTTCTCAAAAAAACGATGACACTGATCAAGGATTGGTTCGAAATGAAC
ACTTATGCCTACAAGATGATCGACTTTCACTCCGGGTATGGGGCATGGAATACGAACCAG
GGTGGGATAAGAGAATTGGCAAAGGCTTGGGAAGAGGAGCTGAAATATGAGTAAGAGAGA
CACGTTCAACTATTCCGAAGCTAGAAGAACAGCCAAGCAACCGGCGAGACTCCTCTATGG
TATACGAGACGGCCGGCTACCAGCTGTTCATGAAGAAGCATTCGAACGAGGGCGAGCCCT
GTACGCCTCTGCTTCCAAGCTGTGTAGCTCGCGCCATGGCTCAGCACGCCTTCTGCGCGG
TGTATCCTGAGTGGTGGGAGGAGACGAATACACTAGAGGCAAGGACTGGGAACAGGTATT
CTTCGACGTTATGTGGGACGGTTTTGTAAGCCCATCCACCCGCTTCTGTCGAACGGTGGG
CCTGCAAGAAGGGCACTACGGTATCTTGTGCTGGCGGTCTGATGGACAACAACCTCTATG
ACCGTTAAGCAATGTAATGACAGAGATCGCAGGTGCGTTAACGAAGCACAGCCATGGCAA
TGCTTTAGCCTGGACCAACTGGCCATCAGAAGGAGACGCTTATCCGAGGCGGTGAGTCTC
TGAAGGTGTATTCATTGGCCCGGGACGTTATCAACGTCATGGAAGAGGTAGTAGCACAGG
GTTCTAGGCGTGGTAGCTGCATACAGCATCAACCCCGCCGGAGATCTCTTCTGGAACGTT
ATCGATCACCTGTATAAGCGCACAGAGTCGAACAACGTCGGATGGCTTCTTAGACGACCG
AACGGTAAGGATATGGCCGAGAAAGACCCGGAGACCTTGAAGCGATGTGGAAGGAATGAT
GTTCGTCAAGCTGGCCAGGAGGAAAAGGCTACTCACGTTTATCGATAAGATGAATCGGCA
CCTTTGCAGAGCCGTTTAAACGGGCGGGTCTACGCTGTGAAGGTCCAACCTTGTCAAGAG
ACTGTTCTCCCGGCAAATGACTGGTATACTTTTAGCTGGGTGTTATCCTGAACTACAACC
TCGAACTCTTTACCGTTCTTGGCCTAAGCACCTTGTGTTTATTGGTCAGGTTATGTCTGA
TTGCAACATCAGCGAATACCCAGCAACCATGGGATGAGTGCGTCAGCGTTCAGACCGCCG
AGCACCGGGAGAAGATTTTACCATCCACCATGACTTCAGGGCGCTTGGCAGCGGTGTTCT
AGGTTCCACACTTCTGCCAGCAGAGCGATTCTCGGTCTGGTAGTATGGATGCTGCGTTCC
TGAACAACGCCATCTTCAAGGGTATGAAGGATAAGCGAAGCCGTAACAGTTGCCAGCTGG
AGGTTCTAGGGGGAGCCAGAGGGCTGCCGTGGACTAGGGAAGCGTAATGCCACTACAATG
ATGATGCCGCCAACTAAATCGACGGCAGAACTCATGGCTAGCTTCTGAGGGCATAGACTG
ACGTTTCGCTATGTGCTTCACCAAGCAGAGCGCTGGAGGTGAATTCTTCCGTGTCAACAA
GGTGTTGCTGGAGATTATCCAAGAGCGAGGCCTCGATTGGGAAGAGGCAAGGCAGATTAA
CGAACGTAAAGGAGGTTCAGCACGTAGACTGGTTGACTGACCACGAAAAGGCTGTGTTCC
GTACCTCGCCTTTGAGGTGAGAATGGAAGACTACCTGCGTCTGTGCTCTCAGCGGCAGAA
GTACATCGACCGCTGTCAGAGCATCAACCTGTACTTCACGTCGAACGACTCGCCGGCTTA
CATTTCGTACATCCATCGTCTCGCCATGGAAGACCCGAACATCCTATCTTTGTACTACAT
CTACAGTATGAGAGGAGTGGAGATATCTCCAGAACAGAAGAATGTGAAATGTGCAAATCA
TTGGCCCGGGGCTTGACAGCTTTCCGGCCTTTGTGTATTCTGAGCACATCAAGGAGGACT
AAAATGAACAGCCCTCTGAAGTGTACTACACTTTGAGCGCAAAGCGTGCTTCCTCGTACA
AATAATATCTTGATAGATAATATGGGATGGACTGCCACAATCCGTCGAGACCACTGGCGG
GATCAACCCTGTATGGGTTGTGGAGTTCACGCGATGAACAGCAAACCCGCAAAGAGTTCG
ATAAATGGTGGGGACAGGACGAGTAAGAAGAACTTCGTAAACTCTGCGCCAAAGGATGGG
CGGAATATATCTGGATGGCTATCGAAGAAAAGTGAGTGTTGAGCTTCCCGCGCCAGAAGG
TGGATTTATCGACATCCAAAAGTACACAATCAGATCGCTGCAAAGAAGCTATCCGCGAAA
CAGGGACTGGAGGTGTTTTAATGAAAGAATATTTTAAAAAGCGACTAATGCAGACCAGTG
AACCGTTCGATTCAGCGACGCAAGCTATGGACGAAGCGATTGCGGGAGGGGCTATACCGC
GAATTCACTTGCGTAACTGTACCCGGGAGCACAATGACGTAGAGCCCGATAAACCGGTAT
TCAATGGACAGGCAACGAATCTTAGTTCCAAACTTCTTTGAGGAGTGGGTAGTATAATGG
AACAAACTATTGATGGGCCCGGTATATTGTGCTTGATAACGAAGGTCGGCACGTTCTGAC
CTTTGTGGCGAGAAGGCCGCCTATAACCCGCTTAGCGTTCAGTGCTGACAAGCATGGTCA
AATCCATGTAAATGGTGTCACAGGCGTTGAACTCGGAGTTCGTCAAGGATCGCTGAAGTA
TTCCTTGTATCCGGCTACATCATCCCAGAAGAGTAAATACATGCAAGTTATGACCGACGC
TTCAAGGCGAGTCGGTGAGTTAGAAAGAGGTAGCCGAACCCGCTAAGGTGGCTATCGCTA
TGTGATATGCGTGTCGTGAAACCCTACTGGAAGATATCCAATGGCGGTATCAAGACGAAG
CTATCTATACTCTACCGGCTACGTTTGCCCTTGTTGCGGGAATCTGCAAACTATGGGCAT
TTCCAAGGCAGCTTCGATAAGAGCCCCCGCCTACCAGCTTCAGTACCGAGCCGGAGTGAA
GCATACAGGCTTCCATGTTGTAGACTCCTCGCTAGGGCATTAACATGTGCTACGCCTGCG
GAGGTAAACTGTACGTCCGAGGAGTATGCACTATCTACTGCCCCTGACTGCGAGAAGGCA
GAGAAAGATAAACCGTTCGTCAGGATGCAGCTTCTACAGGATATCGCGACAGCAAGAGGG
TTGACAACGAATAGAAGTGCTAGTAAGATGGGTCAATATCAGACAGAGGAGGAGCCATGG
CTACAACAGAGAAGATCATCGAGTATGTCTCGGCCTGTATCGAACGCTTCAAAAGAGGAC
GTAACTGGGTACTGGAGAACTTCCGTGACAGGGCGTTATCTAATCGCACAGGGTATATTC
ACGGGCTTATCACTATGGCTGAAATGAATGAGCAGGTGAGTATCGAGGACCGCGACAAGC
TCCGTAAAAGTCGTGAAGCTGCTGAGGCTGTGTTGGAACGTACCTCGACTCCAAAATTAA
TCCAGGGAGGTGCTCTGATGAATAAGGAAAGAGATTGTGCAGGCTTTGATTGTCGCTGTC
GCTCTGACCTCGCAGCAATAGGTTTTGTAGTCGGGATTACTGGGTGTTGGCCAACAAGAG
CGCGCCCGCCGAGGATATTGTCGTGTCTACCGAAGACGGCACCATCAATCTCGGTCGCTC
CGTGAAGAGCGAGAAGCCGCTGTATAGCATGTGCATCAGGTAGAAGACCGCAAACCTGCT
GTGAATTCACCACCAAGGACCGTGTTCTTAGCGAAGTGAAACAGGTTATGCGCCCGGGCG
AGGCTACGGCCCGCGATATTAAGATTGGTAAACGCGGCTGTGACGTATAAATTGGTGAGA
ACGGGCAGGAAGCAACTTTACTACCCCGAAAGGTAAGTATCTCCCGGTTGGCACCCCGTC
TGAAGTACATATTCAGGTATTATTGCGAATCTGTTCGACATGTGTACAATGACTGGCCAA
ACAAAACTTCGTGCTAAAGATGTGAGGAGGACTCATGAGCACAGCCTAAGAAGTAGAAAC
CGTCCGACAAGCCCCAAGGCAGGTGATGTTGTTCTCGACTGCGATCTGATCGGTAAAACC
TTACGATAGATATCCCACGGCAGGCTGGAAGCGATTCCTCCGCAACATGTACGACAAGAC
TAAGACCGTCACCGTCAGCATGGAGTGGTGAAATGGCAAGAACCGTCAGCGCGTAAACTT
CCTACCTGTCAAATTCGGACGGTCGAGTAATCGTCCGATTCGCAAGAAGAGTATTAGGCT
GGCTTGGGGAAACAGGGTAAGAAGTGGCATTACCACCTACGGATGCGAAGGCAAGATTTC
AAATCAGAGGGTTTAAGACACCTCGCCGAGATCAAGGCTTGATTCTCCGGTCCGCAAGCC
TGAGATTACCAATTAGCCGACGAATGGCAATGGTATGTAGGAAATTCACCAATATAAGGA
AAACTAGCAATGACTAGCCGCTGGAAAGAGGCCGTATCGTAAGTACTGGTGCGTCGTTCG
CGCAGGAACTCGAAGAGTTCAAGAAGCTAGACGCTTTGCACCAAATCAACGCATCCGTGC
ATGGTTTGATGCTCATATCGACTTCTAGAACGCCACTTGGCTATTCGGAGAGTCCGGCCA
AGCTCACCCAGACGACCTTTGGTAATTAGGTAATTAGGTGTGCGTTGTAGGTAGAGATTC
AAATCATGGTTATGGCGGGGACGTTAATATCTCTCAGCCCGCCCGCAAGCGCAAGCCTTT
GCCTGAAATTTGTGGTTGACAAGGTTGGCCGGAATATAAACTTACGGCATCCAACATCAT
GAGGAGGGAAGAAAATGCATTCAATATATCGAAGCAACTATGGGTAGCTGCGCGAGTTGA
AGGGCGACTATAAAGGGACGCTTTTGGCATACATGTCTCAAGTGGACTTCAATAAAGATG
AAGCATGCCAGCTTCAAGAAAAAAGCAGCAGACTGGTATGAGCTGGGCAGGTAAGAATGC
CGAGACTTGCTCATAGACAACATCCCTATCTCGGGGCATTCATCGGGGACAGTAAGTTGG
AACGCTGGGTCACTGAGAACAAATATTTCCGTGTGACGACCCTAGGGGCTTTGTTCTAGA
GGTTCCTACCGAATATCTCGACCTTGCTCTACAACCGCACAGTAATCAAGGGAGTTATTC
AAGAGCCTTGCGTGTGGGTTCGATGGGCTCTCAGCACGTTCTACTGCCGGAAGGCTCCTC
TCACAAAGAAGCTTTTCGTAAGTTTCTGTAGAGCGAAAGAGGCGGCCAAAACTGTTAAAG
ATATGGTTGCAGGATGCACCTTCTTAGCTATGGGACAACAGAGGAGCAAAACTAGTATTG
CCCACCTCGACAGGTGAAGATCACTTGCTAACTCCTAGCTAAAAGGGCCGGTACTCTAGC
CGAGGGTGGGCGCGGTCGGTGGGGTTACCACCATCTAGAGAGGGAAATCCTGTGGGATAG
AAAAAGAATCTCACTGGAGAAAGATTCGAAGTGGGTTAGTGTGTTCCGCTAAGCCGGTAG
GAAAGCGGTGGAGTATCCCTGATTTATCCCCAAGTTCCAAAGGCAACCGACATTCAATCT
GGAAGCGTCCCGGATGAACTTCAGAATCTCGACCTAGTGAATGGTGTAAAATGGAGTACA
ACGAGAAAGACGACCGGTCCCACCTCCTATTACCTGTGCCGGAGAGGGTAGTTAAGGTAT
TAGTGTAGATGATCCTCTATGATAATCTGCACAGCTCGGAGGTGCGGTGGACCCGCGGAA
TGGACCGTGGTCGGAGTAGAGTGGCGAAAATAAGCCACCATTATTTAGGAATCGTTCGGC
GAGTACGGGCTTTTCATGGCAGGTTATTTTCAAATTATTTTTTGTGATTTCGGGAGTTTC
GGCCAATATGGGCAATATGAAATTAGAAAGGCCTCAGAATGCTCGCATAAGAGCCGTTCG
AGTGTAGGTATGGTGAGCTACCACTTAGCCGTAGAAGGACAGTGTAACGCTTAATATATG
CTAAATGAAGATTGACAACGGTAGCGCGTATGCGTATTGGCTAGCGGATATGAAAGAGTT
TGTTTCTCAGATGGATGCCACAGAGATTGAGAAGAGTGGGCTTGCATAGCATACCTCTAA
TTAGCTGGTATCAGCGAAGAAAGCAATTCGCACAGAATTAAACGGAGGAATACGAGGCGA
TCTAAGCCAGGGACCGACAGATTTTAGGGACTGGATACCACAGGCTGCAAAATTAGAACT
GGGCAGATTTTTGTGGAACTGGACAGTTTTTTCACAGAAGTACAGAGGAATTTCCCGAAA
TTTAGCCGGATTTCAGAAGGGCGGATCGTTAGCAGGCTATTTCTCCACTGGTTAGCACCT
TTAATCGTTAGCACACCAACTAAAAATTCTAGGACGGACCCTCAGCTGGCACGAACGTTG
CAAGCCCGCACCAGTTTCCCGGCAGGCGTAGCCCCGACTACCCGTCGTGCCAAGTGATAT
GTAGTTGGGGACTCGCACCCCGCGTTTAAGATGCGCCCATTCTCTACCATGACAAACCCC
TTTGTCAACATAATTTTGGCGGCACGTCTTTGCAATTGCATTAGCGTGCCAATTCCAAAC
TTCAGTTCCCTCGGTGTTTATCTCCGGTGCTCGGCCTCCGCCTGTGTTGCCCGCCCGCCT
GTCATCATTACAATAATAAGCCATCACAAGCAAGAAAATTATAGAGAAAATCTACATAAA
ACAGGTTGACAGGCCTTTATATATGCACACGACCACATTCAACGGAACGGCAGGTGTTGC
GCCATGCTACATAGTGAAGCCTCGGGCGATGGTGGCCCCACAAGAGATGGACAAGTGGGA
GCCTCCTCGAATAAGGTTGCACCAGTTTCAACGCAGGATGAAACGACGCGAAATGCTGTA
ATCGCCGCTAAAAGCTCATATAGAGCTAGGTTACAGCTACGTATTCGCCAATGGAGTGCC
TGTGGTATAGATACCATCCGCCATGAAATAGCTCACGCTAAGGCGGGACCGATCATGGTC
ATGATTCCGTATGGAAAGAGTGGTGCGGATTGATTGGCGCTAAACCCGAGCAGTACGCCA
CAATAAAAGGAAAAGTAGTCGTCTAATTGCGGCGACTGGCCATCCACTTGGGTGGCGGCC
GCTTGAACTCACTCAACCATTACGGTTTCAGACGGACTAGCATGGCGAGAAAACAGTGCC
GAGGACGGCCTGAAACGTTGGCAGGTTAATTTGGGTGAAGAACGAAAATAAGGTTGACAC
AGCTTGGGCCATCATGTAGAATGGCCCCGCTAAAAGGGAATCACCCTGAGCACCCACGCA
GGAAAGCAGATATGAAAGCTCTTGTAATCATCGGGCACTCTACGCGGCTTTGGCTAATCG
TATGTATTTGATCGTATTGCGGTAGATTTGAACCGCTACTTCCCTATCGAGAAAGCGAAT
GGGAAGACGGCGTATACATGTATCGTCGCCGCACCTATCTGGTGAAGCATTCGAAGGGCG
CTGTTCCGTTGGCACAGCCTTTACCGCAATGGGACCGTCCAGTGTCGGGTTATCTTTTGA
TCGAAATCATCGAACCTTTCCGCACAGCATAAGGGAGAGATTCAGAAATGCAAGCTTGGA
ACGTATACCAGAATGGCCAGTTGATCGATACCGTTTCTTCGATATCATGTGTGCTGCCGA
AGAGGTGCGGAAATCGTTGGCCAACCATGACGGGTATCCGTCTGATATCACCGTAGAGGA
GGCCGAGTAATGGATAACCTGTCCGTTAAAATCGTTCTGTCGGAACTCAGCTACGTTTTG
ATTCAAGCCTGCCTTGCTTGGCCGAACGCTCGACCTAGACGAGGGTTGGACGATATAATT
TAATCTTATTGCGGATGAGATCGCTTTCCGCGACGGCCTGCCGCCTTTCGCAGACGACTG
CGATAAACAGAATGGATGCTCCGTCGATGGGAATCGCTCCCGGTGGTATCGTGTACAATA
CTGGCAGGACGCACCGATTGTCGCTCCGGTCTATCGAAGTAAAACGGCATGAGGGGAAAC
TGATGACCGTCAGAGTAGGAGATATCGTGGCTATCCGGGGCGACATTCGAAAGCTCGACG
GAACATGCCAATACACGCCCGAACAATTGGTAGGTGGTAAATCTCGCTACGCCGGCATCG
GGAGAAATATGGACTGCCGAGTGAGTAAGATACAAGGTGATATGGCCAAGGTGGAGGGGC
TTGGCGCGGGATCGAATTCTATGTGCTCATTATTAGTGAAATATAAACGCTTGACAACCT
GGACCCAATTAGCTGCAAAATAGCACCCCAGACAACGAAGACGCCGATAGCGAGGAGAAA
GCAGATGCAATGTTTTTCAAACCCGTACCCAAGCTCCGTGCAATGGCCACCAATGGCCGC
AAAGTGGTAGACAACGGCGCTCGCTGAAAAGCGCTGGGCTGTCAAGCTGAACAAAAAAGG
ACTAAGTAATGGCTAAGTCTGTTAGCGTAGACCGCAGCAAGCGATCATTCTCGACGAAGC
GAAGGATGGGAGTTTCTTCTGTGGAGTTTATCAGGCGGACGGCACCGTTCGGAAATGACG
TGTAAACGTCAGATCAAGTCGGCGCATGCTAAACGGCCAGTTTGCTACCCGCAAAGCCCA
CCAGCGCCGGCAAGCCGCACCTGTATACCGCCGCCGAAGTGCTGAACATGGAAGAGGGTA
AATCCGCATTCCGCACCATCAACCTTGAGACCCCGATCCCCAGCAGTAAATGGAATCGAA
TATAATTGATTAAAAGTTTGATATTTCAGGCCACCATTCTGTAGAATGGCCATCAAGCAA
GAATGGCTTAAACAGAAAGGAGATTAAAATGGCACGTTCTATCAAGGCTTTCCGACTTCG
ATTCCGCTTACCAGTTCGAGGCCGCCAAACGTTCCGAACGTAAAGAATCCGCAATCGCAC
AGTCGCTAGCCGAGGCCGCGCTTTCGACTTCCGCGACGATAAAGGTTTGTGCCTAGCCGG
TGGGCCGATTCGCCGGCAATCCTAAGCCCATTGTCGATGGTTTTAGATTCAACCCCTGCA
GCGGAGATACCAAAATGGTTCGTCAATGCGTACCCCGTCCCGATGCTCGCCATGCTTGCC
TGCATGGTTGTTCGCCATGCCGGAGTTCGCCGGTCTGCCCGCTCAAGGACATAAAACTCT
TTAAGACTTCGGGACAGTGGTATGTTAGTTAGACGACAGCGGCTCCCGTCGGGTGCAGAC
CAGACTGAGGATGAATTCGCTGCCGGGACAAGATAGACCCCTACCTTAGCAAGCTTGGCG
AAGTCGATAAGCTAGAGGGCCAATTGCGACGGGTCAAGGATGCAACGGACCCGGATGAAT
ACAACGAAATGGCAAGAGTGCTGAAAGCTCGACGCCGGAAAATAATGCGGCAGAGGGCCC
GGGCCTACGGTCTACCAGAGGAAGAGCCTACCAACGTCAAGGCTACGTCCCGGCAGAAGA
TTACCGCAGGGTGATGAGGCTGAAATGGGTCGAGCCTCCGGATGTTCTGGGCAGTCCTAT
CTTTTCGTCATTTTCGTATGGTTCATCTCAAAAACAGTTGACACGCCACAGTTACTGTAG
AATGGGCGGCACGAGGGATAAGCCCGACTGAAAAGGAACAACGATATAACTGTTCGCGTG
CGTTCTCCATTGAAAACTGGCGATCATCGAAAAAGCTGGCGGAGTGGTCTATACCGCCGG
CTGCCTCTGGCTTCCTACTATGCGAAAGAAAAGATTGTCTAAGGCTTGACAAGTCTCGCG
TTATTTAAAACCAAGAATACATCCGCTCACCACTCACAAGGCGAACCAAAATGAATTTCC
ATCTTAACCTCGTCTCCCTATTCTAAAACTGGTCCGATCCTGTCAGCACGTCTCCACCGA
AGACACTTGTCGGACGCTTGCCGCTCAGGCTAAAAGGCTGCTATGCCTCTGCGATTCGGT
TAATCTCCATTGGAAAAATAAGCAATGGTGAACGTGGCGTAGAGTGGAAAGAGTTCCTTC
AGCAGGTGCGGTCCTCTCCCGTGAGTTTGTGGCGTCATAATCAAGCTGGCGATCTCGTAA
GGTCAGGATGACGGCGTTATCGATAGGATCGCCCTGATGGAACTGGTGAAGGCTAACAAA
GGTCGTGCGCGGCTTCACCTATACGCACTACCCGATGAACAATTTCATGAACCGCCAGCA
CGGCATTGTCGGCCAATCGTAGCAGCTTCACTATCAACCTGTCGGGCAACAATGTAGCGC
ACGCCGATGAACTGGCCGACCTGAACATCGCCCTGTAGTGACTATTCTCCCGATGGACGC
GGAGGAGAATGGCTTTACGCCGAAAAGGACGTAAAGTGGTAACTTGTCCGGCTGAAAGCG
ACAAGGTGAATTGTAAAGTCGTGCGGCTTGTGCCAGAAGTGGCCGACCGGGAGTATATTA
TCGGCCGGTAGCACGGCACAGCAAAAAGAAAACTGTCGATCTCGTACTTTAAGGGTTAAC
ACAATGGACGGGCATCTGTTTACGCACCATTCAATCGGTAGCAAGATGATGGGCCTAAGA
ACGAACGTGCGAATTTTGGCGACCAACGGATGCAAAGGCTGAGTCTTGTATCAGGATCAC
AGAACAGGCACAAGAATTGTCGTATATGACGCTCTAGGGGCGGGAGGGTATGTCAGGTCA
TGGTGCAAAGCGGATGGAGAAATTCACAAAGGAAACTCGCAAAGGCCAGCCAGGGCACCA
CGACAAGGCAGCTACAAGCTAACCGCGTGGGGGGTGAAATTGGTTTCCTCTCAGTACCAG
ACGACAGCAGGCTGCTTACAAAGGAGGCAACTGATGCGACAGCTTAATAAATCCCAGCTA
AAGGCCTTGGGGCCTAAGATCGACCTAATGTAGAGGCTAGCTTCTACTGGCCGAGGAACG
ATCTTATAGACGTAACCCACAGCTCTACCTGCTACATTACAAACAAAGCTGGACCTGGCA
TCTTGCCCGGATCAGCTATCGCAACTCCCGATTCGTTGCATATGGTTGCTGAAGTGTCCG
AGGTGACTGGGAATGTCATTGCAGCATGGGCGGAATGGAAAGGGGTCAACCGGGCCGCAA
TCAACGCTCTGAGTGGTATGGGTATCATCCCTGGCTGGGCATGGGCACAACGATGCTCTT
AAGACTCTGCGTGATATCGAGCGTTACCAGTGACGGCCTCTTACTCGACAGAGAAGCAGG
GCCAGGTGGAGCCATCATCCGTACTGAGCCCGGTTATAGAGGTGACCGGTACGTCTTTAA
ATTCTGTATCAATCAAACACTTGCAGCGCTCTTAAAACGGTAGCCAAAATTCCGGGCTTG
GCAAGCGCTTTAGGCTGGCCTACACACTGAAAGATGACAGAAAAATAAATGCTTGACAGC
CTAGGCCATTTCATCACCAATAGAATGGCCTCAACAAGACAAACCAACCATCAGGAGGCT
AGAAAAATGGAAGCTGTAGCTCCAGTTCAAAGTGAAGATTACGCAAGCGGTGATATACAC
CGCCGCCGTTACAAAGACGTTTGCAGCGCATACGTAGTGAAGCGTTTTCGGAACTAGCCT
GGAGCGAGCTATTGCAACTCCATTATGGGGACCGTTCGAGGATAAAATCTGCAATCGATG
ATGCCGCTAAGTCTGCTATCTAACTGAAAAAAGAACCTGAGGGTAATCTCCGCTATCCTG
TATGAAGGACCAGTTGGTCTAGCTATCAGCCGCATTCGTTTATGTGTGCTTGTGTTATAG
AATGGACTTAGGCGGGACCAACGGTCTGTAGTTATGAAAATTTATCAGGAATTGCTCAAC
CCCGAAGAACAGGACGAATGCTTCCCCACGTTACGCCAAGCTCAGGGCAGAGCTTCCGGG
GTTTATCATAAGGAGGCGTGGGATCCCAGGAAGGTGGGATTTCTTCATTTCCCCATCGGC
CTATTATGCCGAGGTAATATGGTGGAAACTGTCATTGCACAACTAGAGGAACAGGGCAAT
GACTCCAAGCTAACTGGAGCAAAGGCGTCCGGGTTGTCGGCGGAAAGCAGAGTGGGTGAA
TCACACTATTTACCACGAT

In another aspect, the invention provides an SFA1-1 bacteriophage targeting a Shigella spp. Bacteria. In certain embodiments, SFA1-1 binds a molecule of OmpA. In certain embodiments, the SFA1-1 bacteriophage has a genome comprising a nucleic acid sequence comprising SEQ ID NO: 6.

SFA1-1
(SEQ ID NO: 6)
GTACTATATCAATATCAACTACTGATACAGAAAACAACTTGGAGAATAAAATGGAAAACT
TAATTATCATCGAGCAATCTTTCAACGATTATGGTATGGCTTACGGTTATCGTGCGATAA
TGGAAGATTCTCGTGGATGTGTTATCGATATTGCTGAATGTAAAGATTTACTGCAGCTTT
TGAAGATTGTTCGTAAAAATTGGGATTGTGAAAATATTAAAGTTCGAGTTGTTACAGAAG
AAGAAACTGTTTTTCATGATGTAAAATTCGCCAAAGGTGCTGCTACTCTTCTGAAACGTA
TCGCTCCACTGTTCAATTAATGAGGAAATTGTAATGAAACGTAAAATTGTTCAGAATTGC
ACTAATGACGAATTTGAAGATGTATTATTTGATCCAAATTTGGTAGTAGTTCAAAAGGAA
CATACTAGCAAGTTTACTCACTTAACTTCGGTTTATGTGTATGAGAAAGTCGGTGATAAA
CAGCCAATTTACGGTGTATTTCGTGAAATTACTGAAGACGGCACAACTTACTGGAAGGAA
ATTTATTAATGGCTATTAAATTTGAAGTTAATAAATGGTATCAATTTAAAAATAAATAAG
CTCAAGAAAATTTTATTAAAGACCATACTGATAACGGAATCTATGCGCGACGTTTAGGTA
TGTATCCTTTTAAAATTTTAGATGTGGATGCTCTTGGGCGTCCTATTAAAATTTTGTCAT
CTACTGGAAATTTAGTACTATCTTCTGGTAAAGATGTTTTGGATGAAGACTTTATTTGGC
TTTCAAAAAGTGAAGCTGGGTTCTTTGATGAAGTGGAAAATCCATATCAGGCAGCTGAAG
AGCAAGAGCAGGAAGAGCAAGAACAAATAGAAGATTTCACAGAATTCCCGGTAATGAAAG
TTACTATTGAAAATGATGAACAGGCATGGTCCTTGTATCAGATGTTGAAAGCTCACTTTA
AGGAATAATTATGCCGCTTTATGATTATAAATGTCAATCCAAAGACTGCACAAAAGAATA
CGAAAAAATCAAGAAAATTTCTGAAAGGGATACTGATGTATGTCCTGATTGTCATCGGAT
TGCTATTCGGTTAGTTTCCGCTCCTAAGCATGTGAATGGTGGTTTTTACGACTTACTTAA
AGGGTGATTATGTTTAAAATCGGTAAGAAATATCGTATTCGCGAAGGTGAAGAACAGAAA
TTTCTACTTTCTGCTAGTAATAGGAATAGTTCTATTAATGTTGTAATATTGACAAACGAA
TTTATCGTTGAAGATATGAAAGGTCATAATGTTACAATGATTGGTACAGCATCTGGAAAT
GCTGGAAAAACTCTTCATAGTCTGCAAAGTAGTGTTCTAATTTATGATGAAGAATTTGAC
TTTTTCGAAGAAGTCACAGAAGATTTTGATTTTGAATGCACTATTACTATGAAATCTGGC
GACCCTCTTTCCTTTACAGTTAAAGATGAAAGAAGCCGCTTGAGAGTTATTAGTCTTCTT
CAAGCCATTAAATTTGAGTGAAATTATGAAATATGTTAATCGTTCTATCGCAGCATTAGT
ATTAGCAGTGTCTTTAATAGGATGTACTGATGCTGATAATGCAACCAAAGTTTTGTCTTC
AAGTGGTTTTACTAATATTGAAATCACTGGATATAATTGGTTCGGTTGCTCTGAAAATGA
TTTCCAGCATACTGGATTTCGTGCTATTGGACCTACTGGGCAGAAAGTAGAAGGAACAGT
ATGTTCTGGACTATTCTTCAAGGATTCGACTATTCGTTTTAAATAAAAGGCCTTCGGGCC
TTTAGCTTTATGATTACCGGAGTATAATATTCCCGAAACCAAACGAGGATAAGTGATGAT
TAAGAATGAAATTAAAATTCTGAGCGATATTGAACATATCAAAAAGCGTAGCGGCATGTA
TATTGGCTCTTCTGCTAATGAAATGCATGAGCGCTTTCTGTTTGGTAAATGGGAAAGTGT
TCAGTATGTACCTGGTCTTGTTAAGCTTATTGATGAAATTATCGATAACTCAGTAGATGA
AGGTATTCGTACTAAGTTTAAATTCGCAAATAAAATTAATGTTACTATTAAAAACAATCA
AGTAACAGTTGAAGATAACGGTCGCGGTATTCCACAAGCGATGGTCAAAACACCTACTGG
TGAAGAAATTCCTGGTCCTGTTGCTGCCTGGACTATTCCAAAAGCAGGTGGTAACTTCGG
TGATGATAAAGAACGCGTCACTGGCGGTATGAATGGTGTTGGTTCTAGTTTGACAAACAT
TTTTTCTGTGATGTTTGTCGGTGAAACTGGTGATGGTCAAAATAATATTGTAGTTCGTTG
TTCAAATGGCATGGAAAATAAATCATGGGAAACTATTCCTGGAAAATGGAAAGGAACTCG
TGTTACTTTCATTCCTGATTTTATGTCATTTGAAACTAATGAGCTGTCCCAAGTTTATCT
TGACATTACACTAGATCGTCTCCAGACACTTGCAGTAGTTTATCCTGATATTCAATTTAC
CTTTAATGGTAAAAAGGTTCAGGGCAATTTTAAGAAATATGCACGCCAATATGATGAGCA
TGCTATCGTTCAAGAACAAGAAAATTGCTCTATTGCGGTTGGTCGTTCACCGGATGGTTT
TCGTCAATTAACTTATGTGAATAACATTCACACTAAGAATGGTGGCCATCACATTGACTG
TGTTATGGATGATATTTGTGAAGACCTTATTCCACAAATCAAACGTAAGTTCAAAATTGA
TGTGACTAAAGCACGCGTTAAAGAATGTTTGACTATCGTTATGTTTGTCCGTGATATGAA
AAACATGCGATTTGATTCTCAAACTAAAGAGCGTTTGACTTCTCCATTTGGTGAAATCCG
TAGTCATATTCAACTTGATGCTAAAAAGATTTCACGTGCTATTCTAAATAATGAAGCAAT
TCTAATGCCGATTATTGAAGCTGCTTTGGCTCGTAAATTGGCAGCAGAAAAAGCAGCTGA
AACTAAAGCAGCTAAAAAGGCTTCTAAAGCTAAGGTTCATAAACATATTAAAGCGAATCT
TTGCGGTAAAGATGCTGACACTACATTGTTCTTGACTGAGGGTGATTCAGCTATCGGATA
TCTTATTGATGTTCGCGATAAAGAACTTCATGGTGGTTATCCATTGCGCGGTAAAGTTCT
TAATAGCTGGGGTATGTCATATGCTGATATGCTTAAAAACAAAGAACTATTTGATATTTG
CGCAATCACTGGATTAGTTCTCGGTGAAAAAGCTGAAAACTTGAATTATCATAATATTGC
TATTATGACTGATGCTGACCATGATGGTTTAGGAAGCATTTATCCTTCTCTGCTCGGATT
TTTTAGTAATTGGCCAGAATTGTTTGAGCAAGGACGAATTCGCTTTGTCAAAACTCCTGT
AATCATCGCACAGATTGGTAAAAAACAAGAATGGTTTTATACAGTCGCTGAATATGAGAG
TGCTAAAGATGCTCTACCTAAACATAGCATCCGTTATATTAAAGGACTTGGCTCTTTGGA
AAAATCTGAATATCGTGAGATGATTCAAAATCCAGTATATGATGTTGTTAAACTTCCTGA
GAACTGGAAAGAGCTTTTTGAAATGCTCATGGGAGATAATGCTGACCTTCGTAAAGAATG
GATGAGCCAGTAGTTTACTTTACCACAAGGATGTGGTATAATTAATTGGGCAAATGAGGA
TATTGAAATGAAATCATATAAAGTAAATTTAGAACTTTTTGATAAAGCAGTTCATCGAGA
ATATAGAATCATTCAACGCTTTTTCGATATGGGAGAAGCCGAAGAATTTAAAAACCGCTT
TAAAGATATTAGAGATAAAATTCAATCCGACACTGCAACTAAAGATGAATTACTAGAAGT
TGCTGAAGTTATTAAGCGTAATATGAATTAATGAGGAAATTATGATTATCACCACTGAAA
AAGAAACAATTCTTGGCAATGGTTCTAAATCAAAAGCATTTAGCATCACAGCATCTCCTA
AAGTATTTAAAATTCTGTCATCTGATTTGTATACAAACAAGATTCGTGCAGTAGTCCGTG
AATTAATTACTAATATGATTGATGCCCATGCACTTAATGGAAATCCTGAAAAATTTATCA
TACAAGTTCCAGGACGATTAGATCCGCGATTTGTTTGTCGAGATTTTGGTCCGGGTATGA
GTGATTTTGATATTCAGGGTGATGATAATTCTCCTGGGTTATATAATTCATACTTCAGTT
CATCTAAGGCTGAATCTAATGACTTTATTGGCGGGTTTGGTTTAGGTTCTAAATCTCCGT
TTAGTTATACTGATACATTTAGTATTACTTCGTATCATAAAGGTGAAATTCGTGGTTATG
TAGCTTACATGGATGGTGATGGTCCACAGATTAAACCTACATTCGTAAAAGAAATGGGTC
CAGATGATAAAACTGGTATTGAAATCGTAGTTCCAGTTGAAGAAAAAGACTTTAGAAACT
TTGCTTATGAAGTTTCTTATATCATGCGACCATTCAAAGATTTGGCTATCATTAATGGTC
TTGACCGCGAAATTGATTATTTTCCGGATTTTGATGACTATTACGGTGTAAATCCAGAAA
GATACTGGCCTGATCGTGGTGGATTATATGCTATCTATGGCGGTATTGTTTATCCTATCG
ATGGTGTTATTAGAGACCGTAACTGGCTAAGCATTCGCAATGAAGTGAATTACATTAAGT
TTCCAATGGGTTCACTTGATATTGCTCCATCTCGCGAGGCTCTTTCACTGGATGATCGTA
CTCGTAAAAATATTATTGAACGAGTTAAAGAACTCAGTGAGAAAGCATTTAATGAAGATG
TAAAACGATTTAAAGAATCCACATCTCCCCGTCATACATATCGTGAATTGATGAAGATGG
GGTATTCTGCTCGAGATTATATGATTAGTAATTCAGTCAAATTCACGACTAAAAATCTGT
CATATAAAAAGATGCAGAGCATGTTTGAACCTGACAGTAAGTTATGCAATGCAGGAGTTG
TGTATGAAGTAAATCTTGACCCTCGACTGAAACGCATTAAGCAAAGTCATGAAACTTCAG
CCGTTGCATCAAGTTATCGTCTGTTTGGTATTAATACAACAAAAATTAATATCGTTATTG
ATAATATTAAAAATCGTGTTAATATTGTTCGTGGATTAGCACATGCGTTAGATGATAGTG
AATTTAATAACACTTTGAATATTCATCACAATGAGCGTCTTCTGTTTATTAATCCAGAAG
TAGAATCGCAGATTGATTTACTTCCTGATATTATGGCGATGTTTGAAAGTGATGAAGTTA
ACATTCATTATTTGTCAGAAATTGAAGCTTTAGTTAAAAGCTATATTCCAAAGGTAGTTA
AAAGTAAAGCTCCTCGTCCTAAAGCTGCTACAGCATTTAAGTTTGAAATTAAAGATGGGC
GATGGGAAAAAGAGGAACTGTTTACGCTCACATCAGAAGCAGATGAAATTACTGGTTATG
TAGCGTATATGCATCGTTCTGATATTTTCTCTATGGATGGTACTACATCTCTTTGTCATC
CATCTATGAATATTTTGATTCGTATGGCTAATCTTATTGGTATTAATGAATTTTATGTTA
TTCGTCCGCTTTTACAGAAAAAGGTAAAAGAACTCGGTCAGTGCCAATGTATTTTTGAAG
CTTTGCGTGATTTATATGTAGATGCTTTTGATGATGTAGATTATGATAAGTACGTAGGTT
ATTCAAGTTCAGCTAAACGATATATTGATAAAATTATCAAGTATCCTGAGCTAGATTTTA
TGATGAAGTACTTCAGTGTAGATGAAGTTTCTGAAGAATATACACGACTCGCTAATATGG
TTAGTTCATTACAGGGTGTATATTTCAACGGTGGAAAAGATACCATCGGTCATGACATCT
GGACAGTAACTAATCTTTTTGATGAATTATCACATAATGCTTCAAAAAACAGTGATAAAA
TGGTTGCTGAGTTTACCAAGAAATTCCGTATTGTTTCCGACTTCATCGGATATCGCAACT
CTTTAAGTGATGATGAAGTTTCTCAAATCGCTAAAACTATGAAGGCCCTTGCGGCCTAAT
AAGGAAAATTATGTACAATATTAAATGCCTGACCAAAAACGAACAAGCTGAAATTGTTAA
ACTGTATTCAAGTGGTAATTACACTCAGCAGGAATTAGCTTATTGGAAGGATGTATCGGT
TGACACAATCCGTCGTGTTTTGAAAAATGCTGAAGAAGCTAAACGCCCTAAAGTTACTAT
TAGCGGTGATATTACAGTTAAAGTTAATAGCGATGCAGTTATTGCTCCAGTTGCTAAATC
TGACATTATTTGGAATGCATCTAAAAAATTCATTTCAATTACTGTTGACGGTGTAACTTA
TAACGCAACTCCTAATACTCATTCAAACTTCCAGGAAATTCTTAATCTGCTTGTAGCGGA
TAAGCTGGAAGAAGCTGCGCAAAAAATTAACGTTCGTCGCGCTGTTGAAAAATATATTTC
CGGTGATGTTCGAATTGAAGGTGGAAGCCTGTTCTATCAAAATATTGAATTGCGGTCTGG
TTTGGTTGATCGTATTCTCGATTCGATGGAAAAAGGTGAAAACTTTGAATTTTATTTTCC
GTTCTTGGAAAATCTGTTGGAAAACCCAAGCCAAAAAGCGGTATCTCGACTCTTTGATTT
CTTGGTAGCAAACGATATTGAAATCACCGAAGATGGTTACTTCTATGCTTGGAAAGTAGT
TCGTGACAACTACTTTGACTGTCACTCAAACACCTTTGATAACAGTCCGGGTAAAGTAGT
TAAAATGCCACGTACTCGTGTGAATGACGATGATACACAAACTTGTTCTCGTGGTTTGCA
TGTGTGTTCTAAATCTTATATTCGTCACTTTGGCAGTTCAACTAGTCGAGTTGTAAAAGT
TAAAGTACATCCGCGTGATGTAGTATCAATTCCGATTGATTACAACGATGCTAAAATGCG
TACCTGCCAATACGAAGTAGTTGAAGACGTTACTGAACAATTTAAATAAGGGCTTCGGCC
CTTATCGCATTAAGGAAAATTATGTTAGGTTATCAAGCACGAGTAAAAGAAGAATACGAT
CAATTAATGCTCAAAATTAATGCACTGAGCAAATTTTTAGAAAGCACAAAGTTTCTAACG
GTTAGTGCAGTTGAGCAAGAATTACTGCTTTCACAGTTCATCTCAATGAAATCTTATGCT
GAATGTCTAGAGAAAAGAATTGCACAATTCAAATAAAATAAGGGCTTCGGCCCTTTTGTT
TTAAGAGAAATTATGATTTATATGAATATCAGTGATTCCGACATAAAGGAAAGTTTAAAT
GCAGAAAACGAATCCTGGGTTACAGAGACTATTTCAGATTCCGACATTTACTCTATCGAA
CAGCGATTTAACCAATGAAATGAAGGTCAAAATTGCTGATACAGCAAGATACTCTTTAAA
ACAAAATCCGAACCAGGATAAGGCTGAGGTCATCGAAAGATGTCGTATCGCAGTGTATGC
TGAGTATGTTGTAGCAGATTGGTTAAGTGGGTATGTTAACAAAGGTCAAGAAGACGTCGA
TGATCCGTACACGTATGCATGGGATGTTTTAGCACATCCAAAATACTGTGGGCTCCGTGT
AGAAGTTAAGACACATCAAACTGACTCACGTTGGATTTCGGTAACAACAGGATGCAGCGG
AGAGTATCCATATGGTTCTGGAATAAATCTAGGGCCCATTCTAAATCATCAAGTCGCGGA
CTGTATAATTATATTCAACACTAAAGAAATTCATCCAGGTGTCATCCAGTACACTCCGAA
GTTCATCGGTGACAGAGAAGACCTTCGTAAGGTTGTAAGAAAAAGCAACTACAACGGATG
GTATCTTTCCATTTAAAAAATTTCATAAACTAGTTTACATACCACAAGGACCGTGGTACT
ATACAACTATCAACTACAGAGGAACAAAAATGAAATTTAAATTCTTTTATGTTAAACATA
AAATCACAGGTGAATTTGTTCATGTACAATATTCTGAAATTGACGATGCAACATCATTTA
TTTCATTGGGATTGTCTAAATATGAATCAGACCAACCATATCTTACTGATAAATTGCAAC
AAGAGCGTTTGTTGAATGGTGATTTAAACAATTCTTGGGAAGTTCTTATTTACGATGATT
TAGAGAAAGCTATTAAAGAAAATAATCTTGAAATCATTGAGATTGAACTATGAGTTCATT
ATGGTGGTGTTTTGTTTGGTTAATTAGTATTCCAGTAATTTGTTTAACATTTACTTTTGT
GATGAGGTTATTATTAAAATTTTGAATTCTGTACTTATTGCTTGTGCGTGGTGGGTTGTG
CAAGTTTCAGCGGTAGTAGTTGGTATTCACATTTATTACGAATATTTTTAAAAAAGTTGT
TTACAAGACTGTTCCTCTGTGGTACTATTACCCTATCAACTACGGAGGAACAGAAAATGA
AAAAGATTGTTAAAGCTATATGGAATGTAGTTATAATACTAATAGTTTTGAGTATATTCC
CAATCGTTTTAATGATTGATGTATTAAACGTTTACTTTGGATTTATGTGAGGAAAATATG
AAGCGTAAACGCAGTGCTTTTACATTTATTGAATGGTTTTCGATAATATTTTTCCGGCTT
TATTCATTTTCATGCTGATTTTTGCCTTAGGTTCAGTTGTAGTTGGAATCTATTTGATGA
CAGTAGTCGGAATTGATATTCATCAAAATGGTTTAAAATCCGTAGTTGAAACAATTTGGA
ATGGTGTAAAATGATGAATTTGCTGAGCGGTTGGTTTTATATTCTCATGTTTTACATTGG
CGCAAATTTCCCGTATTGGATGGGATGGTCAACAACTGCATTTGGATTTTATACTCCTTG
AGGTGAATTATGAAAATCTTTAAAGATGTAAAAGTTGGTGAAATTTTTTGTTTAGATAAT
GGTGATCAGTTAATTCGTATTTCACCTCTTAAGAGCACTAGCGAGAAACCGACAGTTAAC
GCTACTTTAGCAAATAACAGTAATGAACGTTTCTGTATTGAAAATGATACTGAAACTTAC
ACCGTAGAAGAATTCTGGGAATTAAGCGTCGCAGTCGACGCTTAATTTATCGGCCGTATG
TATTCATGCGGCCTTGGAGTAGAAAATAATTTAGAGGAAATTAAAATGAAATATATGACT
GTTACTGATCTGAACAACGCAGGCGCTACTGTTATTGGTACAATCAAGGGTGGTGAATGG
TTTTTGGTAACTCCGCATAAAGATATTTTATCTAAACCTGGATTTTACTTTTTAGTAAGT
GAATTCGATGGTTCATGCGTATCTGCACGATTTTATGTAGGTAATCAGCGTTCTAAGCAA
GGATTCAGTGCAGTTCTAAGTCATATTCGCCAACGCCGGTCTCAACTTGCGCGTACTATT
GCAAATAACAATGTTCCATACACAGTGTTTTATCTGCCTGCTTCTAAGATGAAACCTCTG
ACAACGGGATTTGGAAAAGGTCAGTTAGCTTTGGCGTTTACTCGTAATCATCATTCTGAG
TATCAAACACTTGAAGAAATGAACCGTATGTTGGCTGATAACTTTAAATTTGTTTTGCAG
GCATATTAATGAGTAATTTCCACAACGAACACGTGATGCAGTTCTATCGTAACAATCTTA
AAACCAAAGGCGTCTTCGGACGCCAGTGAGGAAAATATGAATATCGCAAAATTATTAGGA
GTTATTTCATTTATTTGTTGGATAGTAGCATGTGTTTTAACTATCTGTATTGATGCCAGC
AGTGTGTTTTCGCAAGCTTTAGCTCAGGGTATGTGTGCATATTTAACATTTGTGTTGTTA
TCTACTAATGATTAAGAAAATCTTGGGCTATTCATTAGCCCTTGCTATTTTATTGATAGC
ATTATATTACGGAATAATGTTCGGATTAATTCAAGTCGTGCTTTTCATTTCTGATGTTAT
TATGGCACTACATTCACTAGTATGGTAAATTTATGCAACTGAATAATCGCGATTTAAAAA
GTATCATTGATAATGAAGCATTGGCTTATGCTATGTACACGGTTGAAAATCGTGCCATTC
CAAATATGATTGATGGATTTAAGCCAGTTCAACGATTTGTTATTGCTCGAGCTCTTGATT
TGGCACGAGGAAATAAAGATAAGTTTCACAAACTCGCTTCTATTGCAGGTGGTGTAGCGG
ACCTTGGATATCATCATGGTGAAAACTCTGCGCAAGACGCAGGTGCTTTGATGGCTAATA
CTTGGAATAATAACTTTCCTCTATTAGATGGTCAAGGAAACTTTGGTTCTCGTACCGTTC
AAAAAGCAGCAGCAAGTCGTTATATTTTTGCTCGTGTAAGTAAAAATTTCTATAACGTAT
ATAAAGATACTGAATATGCTCCGGTACATCAAGATAAAGAACACATTCCGCCTGCTTTCT
ATTTGCCTATTATTCCTACTGTTCTTCTTAATGGCGTTTCCGGTATTGCAACTGGTTATG
CAACTTACATTCTTCCTCATAGTGTTTCTTCTGTCAAGAAAGCTGTACTGCAAGCTCTTC
AAGGAAAGAAAGTAACTAAACCGAAGGTAGAATTCCCAGAATTTCGCGGTGAAGTCGTTG
AAATTGATGGGCAATATGAAATTCGTGGAACATATAAGTTTACTTCACGAACTCAAATGC
ATATCACTGAGATTCCGTATAAGTATGATCGTGAAACTTATGTGAGTAAAATCTTAGACC
CACTTGAAGATAAAGGCTTCATTACATGGGATGATGCTTGCGGTGAGCATGGTTTTGGAT
TCAAAGTTAAATTCCGCAAAGAATATTCTTTGAGCGATAATGAAGAAGAACGTCATGCAA
AAATTATGAAAGACTTCGGGTTGATTGAGCGTCGTTCTCAGAATATTACGGTTATTAATG
AGAAAGGAAAACTACAAGTTTACGATAACGTAGTTGATTTAATTAAAGATTTCGTTGAAG
TTCGTAAAACTTATGTCCAAAAACGAATTGATAACAAAATTAAAGAAACTGAATCAGCTT
TTCGTTTAGCCTTTGCCAAGGCACATTTCATTAAGAAAGTAATTTCAGGTGAAATTGTTG
TTCAAGGTAAAACTCGTAAAGAACTGACCGAAGAACTTTCTAAAATTGAGATGTATTCTT
CTTATGTTGATAAACTAGTCGGAATGAATATTTTTCATATGACTTCCGACGAAGCAAAGA
AACTTGCTGAAGAAGCTAAAGCTAAGAAAGAAGAAAACGAATATTGGAAAACTACTGATG
TAGTTACCGAATACACTAAAGATTTAGAGGAAATCAAATGAGTCCATTCATAGGTATTAC
AAGCGCTGCATTAGTATCCGGTAGCATTTTACTGGCGGGTTTAGGTGTTGTTCCAGCCGT
AGCAGGAGGTCTTCTTGCGTTCTGCATTCAACGTGTTATCATGACAGTTATCACAGTCAT
GCAGTAATTTTAGGGAGAGCCGAGGCTCTCCCTTTTTTATTTCAAAAATTTTTTTCACAA
AACGGTTTACAACCAAAGCATACTGTGGTACTATACAACTATCAACTACTGATACAGAAT
TACGGAGATTAGAAAATGTCTAAAGTAACTTACATCATCAAAGCTTCTAATGATGCTCTG
AATGAAAAAACTGCTGCGATTTTAATTACCATTGCTAAGAAAGATTTCATTACAGCTGCA
GAAGTTCGTGAGGTGCATCCAGATTTAGGTAACGCAGTAGTTAATAGTAATATTGGGGTA
TTGATTAAAAAAGGCCTGGTGGAGAAATCTGGTGATGGATTAATCATTACTGGTGAAGCT
CAGGATATTATTTCAAATGCAGCAACTTTATATGCACAGGAAAATGCTCCGGAACTGCTG
AAAAAACGAGCAACTCGTAAAGCTCGTGAGATTACTTCTGATATGGAAGAAGATAAAGAC
TTCATGTTAAAACTTTTAGATGAAAATGGATTTGTTCTTAAAAAGGTTGAAACTTATCGT
AGTAATTATCTCGCTATTTTAGAAAAACGCACTCACGGAATTCGTAATTTTGAAATTAAC
AATAATGGAAATATGCGAATTTTTGGATACAAAATGATGGAACATCATATTCAGAAATTT
ACTGATATCGGAATGTCATGTAAAATCGCTAAAAACGGTAATGTGTATCTTGACATTAAA
CGCTCGGCAGAAAACATTGAAGCTGTAATCACTGTAGCATCTGAACTGTGAGGAATAAAT
AATGAACAAGTTAGAAATTGTCAATGAACTTCGTCGTTGCGCAGAACCCACTCAAGAGGG
TTGGGACATCTGGTACCATGGAGCTTATCTTGGAACTATTGCTAAAGTTAGAACTGGGCT
ATATTTCATTATTCGTGAAAATTCTGAATTACCTAACGGAGTAGCTTATGGCGGTGTACG
TAAAAACTTCATGGCAGCGATTAGTACCTTTGTTCAATCGGCTTACGAAATTTACCTTGC
TGATTATAAAGAATTCCAGGAATCTCAACCAGTTATTCGCTCAATTGGTGTTAACAAAGC
TCAACAGAAAACTTTGTGGCAGCGTATTAAAGGATGGTTTAAATGAACCCATTTATTAAT
CGTTTAAAAATGCTGAATGTTCCTTTATCTCGTGAAACTCCAGAAAGTTTTGTTGAAAAA
TTTAAAGCGCATGGTTATAAATGCACAGAAGAAGATATTCTAAAAGAAGTTCCTGAAATA
TGTTGGCAGACTGCATATTGGGATGAAAACCAAAAGTATCAACGACGAATTGTCTGCGCG
GCCAACCGTTTTAAATTAAAAGATGGACGAACTCTTATTATTCCAGGTGCTCGTCATTAT
TCTAAAGATATGGCCGAAGTTTTAGATGTAGTTAAACCTCAATTAGTTACTCAACAAGTT
TGTGATGATGACCAAGGGTTTATTGACCAATATAGTAATTATTGGACACGTGAAGAAGCA
ATGATTATTGCAACTTACGCTGGACAAGTACGTATTGAACGCGGTGGTAGTGAAAAAGAA
CTTTACTCTGAGGACCTTTACTAATGAATATTAAAAAGTTTCAAATTGATGGAATTACTA
ATCAAATCCAGGCACTGGAATATGCTAATAAAATGATGTCAAGCAATTGGGGAATTTATA
CCAATGAGCCGGGATTTAAGTTCTGCGATATGGAATTCACCAAAAAGCTCGTAGGAAAAG
ATTATGTATGCCCATTTAGTTCTCCGGTAAATGGAATGCTAAAACCTGCTTTACGCGATC
TTTATATTGCGATGAACGAAGAAATGATAGAAGAGCTAAAACGTCAACTGAAGGTGATTC
AATTTGGCCAGGGAAATTAATTCAAAATCTGATTATTTTAATTCTCTCAATGATAAAGAT
AAAAATCTAATACGGCATTTTATTGTTGAGATGGGATATACCGATACACGCGATTTAAGA
GAACATATATTTGAATGTGGTGTAGCTAAAAAGTTTTCATTCACATGCAAATGTTTAAGA
GAGGTAATTCAGCACTATGAACAATTTAGTCGCAAAACATAATTTTAATAAAGCTTCTGT
CCATAAAGATAAGAAGAAAGCGTTTAAAGAATCTAATCGCAAACAGAAACATAAGGGGAA
GGTCTATGATTATTGATTCTCAATCCGTGGTTCAATACACAATCAAAATTGATATTCTAG
AAAAGCTATATAAGTTTCTACCAAATTTATACCACTCAATTGTCAATGAATTAGTTGAAG
AACTACATCTCGAGAATAATGATTTCTTGATTGGAACTTATAAAGACCTCTCAAAAGCAG
GATATTTTTACGTAATTCCAGCTCCAGGAAAAAGTATTGATGATATATTAAAAACTATAA
TGTTTTATGTCCATGATTACGAAATTGAAGATTATTTCGAATGAGTCATAATCTTGAAAA
TGTAATCGAGCACAATGTAGCTCAGGAACGTGAGTCGTTCAAGGAATTCGTAGAAAAAAT
TTTTGAAGAAAATAACACAGACCGGTTTACAAATCAAGCGTCTGATGATATTATAACAAA
GTCAACTAATTGAGTGGTATAGTTAATGAATAAAAACATTGATACAGTTCGTGAAATTAT
TACTGTTGCGTCTATTTTGATTAAATTTTCCAGAGAAGATATTGTTGAGAATCGTGCTAA
TTTTGTTGCATTTCTGAATGAGATTGGAGTAACGCATGAAGGTAGAAAGTTAAATCAGAA
TTCATTCCGTAAAATTGTTTCTGAATTAACTCAGGAAGATAAGAAAACCCTCATCGACGA
ATTCAACGAGGGTTTTGAGGGTGTATATCGATATCTAGAGATGTATACGAACAAATAATT
ATTTAGCCCTTCCTAATATTCTGGCCGCCTGAGCACATATTGATTCAAGACGGTCATTAC
TTATATGATCATTTCTATACCAGTACATGGTTATCGTTCCAGCATAGATATTATCCAAAT
TAAAATATGGACAACTGTACATGTAGTTTATTTCGGGAGTAGGCTTTTTAGTTGGTAAAA
AAGCAAATTTTGAGTTTGAATAATAATGACGTCCATTTAAATGAACTGCATATTCATCCA
TAGTTTTATCAACAGGATATCCTCCAAGTGATTTTTCAGTTATTGTTGAAGGTAATTTTC
CTTCGTATGCTATAATATCAACAAAATAGTTTAAGTTTTTAGGGCGGAAAGAATACACCG
CACTAAAGTCTGCCTCAGATGATATATGAACTATCTGAAGTTGTTCCAGGGCGACAGATT
CAAAGCGTGCATTTCTTTCCTTTTCAATAATTTCACTGTATGTTTCATACTTTGATTGCT
TATAGTACTCAAAGAAACTATCTCCCCTATACCAAACAATCGCCATTATAAACAAAAGAA
TTACGACAGCTACCCGGGAAGCAAGAACTTTCCCGGTAGCATTATCTTTGAACAAGCGAT
CTAGAACACCCAACAGAATATCAGAGGGCGAAAATGATATTCTAGGTGCTGCCATAGACC
CTCCTTTTAAGGATATTTATTCAAATTATACTCTTGGTCCGTAAATTGTTCCAACAGCAC
CCCAAGTTGGAGAAGATCCAAACACTGCATGACCAGACGCACCACCAACACCAACTTTAA
GAACTTTACCGCCGTCAGACCATGCATTCTGACCAGCAGCACCAGCATTACCACCTGCGC
CGGAGTGTGCGTTACCATAGTCATAACCACCACCCGGACCTTCATACGAAGCAGCACCAC
CGGAATAACCGCCGCCGCCAGCACCACCGAAAGGTCGACCGCCGCCGCCACCGATATCTT
GCCAGTTTGCCCCTGAGTGTGGACGATATGCAATACCACCGCCGCCGCCACCGCCAGCAG
CTATAACACCGTAGTTCCAAATACGAAGCCGTCCACCGATATCATTTTGAATAACATGAC
CGCCTTGTGTACCGTTACCGTCGCCTCCTGAGTGAGTGTATCCACCATTACCACCACGAC
CCCACATATGAACACCACCATGAATATTAAGAACAACATATTCATTAGGCGTATCCCCAT
ACATAAAGAAAAGTGGCACATCTTTAGAATAAGAAACGATATCACCAACAATATTGAATA
CAATGGGTGCACCGCCAGCTTCAAAGCAACGGTCGCGGAACCATTGACCATTAAAGTTAT
GGTTTGCTGTTACGGTATGAATAATCTCTCTTGAGCGTCCGGCGAATTGGCTCATCCAGC
AAGGAACACCCAATCTTAATTGACCAGCCGCTTGACTCATCCATCGTTGTCCTGTTTCAT
TTACGGATGAACTTCCAATCCAACCGGGAACACCAACTACTGCCATAATTTTCTCCAAAG
GGGCAAAGCCCCTTGTTGTTAGAATTTAAAATAATTACTAATTTTGCGAGCAATTTTAGT
GAAGAAAGGCGTTTTCAATTCTTTAATTTCTTCGCTCATTTCTTGGATAGCCTTAATTAA
AAGAGCATTCACAGCGGAGTTAGAAATTGTCAGAACATCAGATCCATCAACTTCAACTTT
AGACACAGCTTCCGGTAATTCTTTTTCAAGATCCTGAGCAATAATACCAACTTCACGTTT
AATAACACTACGATCTTTAATAGACTTAACCTTATCGTATGTATAAACTTTCAGTCGGCA
TACTTTTTCAAGTGCTCCGTTTTCTAGCTCTTTTTTATTAATCTTAAGACGACCATCGGA
ACGAATATACACATCGTTAAAGTTACCATTACCAGGAGAATAGAAAGTACCGTCGTAATG
AAATTCAAATACCGCGTTAGGGTGTCCAGCCGCTGGCGATGCCTCCGCAGATCCAACACG
AATAATTGCTTGTCCCCAACTGTTATTGACACGACGAATACCAAGATCGACGGCAGTTAC
AAACCCTTGATTGGTGATTACACTTCTTCCTTTAATAATTGGATAATAACAATCCTGTGA
AACATAACCATGATCGACGAATATTGGCGCTTCCACTTGCCACTGACTACCCCAATCACC
TGGTTGACTAGCAGTCCAACGTTCACCATGAACATATAATCGAACGTTATTGACATGGGC
CATTTCGACGCCTTTTGGACTTAGCGTGATATCTCCTGCGTTTGTAATGTATACACCAGC
ATTAGTAGCATAACTATATAACGCTAATTCATTATCAGCACCACCCTTACCAATATACCA
GTTACCAGTCCCGTCTATGTCACCACGAATACGCAAAGAATTACTAGCAGTTGTTGATAA
TACAATAGAATCACCCGGACGTTTAATAACCAATTTACCATCGGATTCAATTTCACCAAA
AAATTTAGTCTTAATTGCACCAGTCGCTAAAGTACGTTCAGTGGAAAAATGAGCGCCTTT
GCTGTCTTCTAACTGAATAATGTTATCACGCGCATTATTACTATCACCCCAATGGTTAAA
TTTAAAATAATCTCGTGATCCTAAACGTCCGGCGTCAAAAGTCATCGTGCCTTTAATGTT
AATATCACCGCCAAAAAATGCGGTGAGAGAATCACGTTTTAATTTTAAAACGTTAGCACC
ACTAGCACCACCTAAAGTAACATCGCCAGTTCTTAATTCATAACGGAAAGGTCTATTTGT
ATTAAAGTTTCCATCTTGATCATTTTCGTCGGTGGAAAGAATATAAAGACTCTCTCCATC
ATTACGGAAAATTGTACCAAATTTTCCGCCATACATTTTAAATGCATTCAATGAACGAAA
TTTAACTTCTGTATTAAAAGTTGTAGTTGATTTAAACGTGGTTGCAGCACCGAAGTTAAC
TGCGCCATTAAAATCTTGTTGAGCAGTCCAAGTATGTGATCCGTCTTCAAGAACAACGCG
TCGAACTGTACATGTTCTACTCCCTGGATTACCGAATACACGAACGACAAAATAACGATA
GTTTGTCTGGGATACAGTTCCACGCCAAACCTGAACAGTACGTCCTGTGCTGGTGTGCTC
GCTAGGACCAACAGAAAACATAATTAAGTTTCCGTCAATCGTGCCCCAATTCATATTAGC
TGGCATGTTCTTTATTTTGTTCAAAGGAACACTAAACATACTACCAGGGACAAAATCAAA
GGTTTGCCAGTCTAACTCAGCTAAGTTAGATTTAATACCGCCCATACCCAAAGTAATTGG
TAGAGAATAGGATGTGTATACTTCGTTCCATTCACCCCACACACCGCCGGTATATGCGCG
CTCAAAAATACGATCGCGGGTAGTTGCACCTGTTCCGGCAGTAGTAAATTTTTGGAAAAT
AGCACCACCAGAAGCACGCTGTTTGACTTCCAGCAAACCAACAATAACGGCTGGTAAACC
TTCACCAGTAGGACAATTAATAACGGCATCAGTGCCTAAAATATTATATAACCCAGGAGT
TTTAAAATCATTCAGGTTTCCGTCATAGAAAGTCGCTGGTGTGACATTACCGACTTGTTG
CCACGGTCCCCATTTAGGCGCGGAAACATCCCACGCGGCAGCAAGACAACGAGTATATAC
AACACCAAATCTGGTTGTATATCTTTGTGTACGAGAATAGTTTCCACCTTCATATACTTC
CAAAAGACCTTGAGCTGACGACCCCTCTTCTGGGAAATTTCTGTCAAAGGTAGCAATGGA
ATTTGAACTATTTCGCCACAAACCAAGGTGTTCAGCGCCTCCTAGATCATTAAGATCAAT
TGTTGTTGACAGTGGACGAGTACCGGATTGAATATTTCGCCACACACTCCACGGACCATT
TACTCCGTTCCATGTAGCAGTCAAGGAACGAATATAAACATTTCCGTATCTAGTTGTATA
TCTCTGAGTACCAGCAAATTGACCGGCCACAAATACTTCTAGCACACCTACAGCATTTTC
TTCTGGAAATTTATTTGCTGGTTGAGCGTTGGTTGAAGTTGCTTTCGACCAAACTCCAAG
ATATTCTTCAACAGGCCCATACGTATCTAAATTAGCATCAGTAGGTAATTCGCCATTATT
TTTTATAAATGTTGTGCTATTGACATTTATGTCAGATGGAGTTAAGGTAATGTCCTTTGA
CCCATCAAAAGATATACCATTAATTTTCCTAGGTGTTTGCAATTTAGTAGCTGTGCTAGA
ATTTCCAATTAAAGTTCCAGTTATATTTTCTGTAACCTCTAAAGACCCATTTATAGTTCC
TCCATATTTTAAACCTAATTCTATAACACTACCTGAGTCATCTTTTGTGAAAATTGTTTT
ATCTTTTAAGTTTATAGCCAATTCACCTTCGGCTAATACTGAAGCAGCAGGACGTTGACC
TGCAGTTTTGCTTCTTTTAAATTGTATTTGTTTTAAAGTAGCCATAAGTCCTCTTAATAA
TAGCCGAAATCTTGAACAGAATCCTTAATTACGATTTGGTCAAATCGTGGAACGTGTGAA
GGTTGAGATGCAGGATTCTGTGAAAAAAGGTTTGGCGCAGTTAAATTTCCTGTCATAGTG
TCTCCAGAGCGTAATACCCTAGAGTTTGCATTTGCTGTAACAGTATTTATTGCTCCATCA
ACATAATCTTTGCGAGTAACATCAGATGCTGCGGAAGGAGTAGATGTAACAGTTACCGAT
GGAGCCGTTACAGCCCTGGTAAATGTTGTCAAACCTTCTGGCGTAATAGTAATTTGTCCA
GTAGTGATTTCACTACCTGCAGGTCTAAAACTAATACCACCGGAAGAGCCGCCAACTACA
AGAGTATTACCAGTGCCGCCACCAGAACGAATTCGGATAGGGGTTTCATTATTACTTAAT
TGAAGATAACTATTTACCCCGTAATTAATAGTAATTGGACCGGCGTAAGTACCACCATTA
GCTTTAGAAACGAAATCGTTATCAGCTGCCTGCGGTTTATTATATTCTGAATATATTTTA
AATGATTTGTAGAGTACATCGTCACCGGCTGAATTCAATGGAAAATTTCCTTGATGCCAA
ATGACAGATCCACCCACAGTTGAACCTACTTTTAAATCAGCCATTATATGCCCCTTTATT
TTAATATTATTTATAAAGAAAAAGGGAGCCCGAAGGCTCCCTCAGTTTAAACTTCTCTAA
ATTCTTGCCCAAACACTTTGCCAGTTTTAGAAGAGGCTTGTGTAGGAAGTACCATTATAT
CTGGAGGTGAAGCCGATTCACAGACATAATTAACGCGAATACCGTTGACGCCAAATTCGA
CAGGTTTTGAAATGCCGCCATTTCTTGATACTTCAGAAAAGCTTAAATTCCTCATTCCGC
CTTGACCAGCTTGAGCAGTTCTCTGCGCATATATAGTAAATCCGACTGCATTTTCAGGAA
CAACTACATAATCTTCCTTTAATTCCCAAGATCCAGCTTGTCCAGTAAACTCAGCTTGGG
TAGAGGAAATATATCCATTTGATGCATCATAAAAACGAATGGATATGTTTGTAGTTCCAA
GATCAAGTAAATCAGCATCAGCATATAACTGTGCTTTAAGATAAAGAACATCACCAGGAA
TTAAATTATAGTCAGAAAGTTTACTTATAGCAGCTGAAGTTGGCAATCGCGCAATTTCGT
TATTAGTTCCACCAACCGCTGACATAAATTGCTCGACACTTTCATATGTTCTTTTAGGAA
ATCCCGTAGCTCCGACGTCTTCTAAACTATCAAATACAACATCTAAAATAGTTTGATAAT
CATCTGTGCTTTTTCTATTACTTAGTTTAACATGCTCTAATGCAATAGCTCTTTTAGAAG
AAGTATAAAAAGCAGCATATGATACGTCAAATCTTGACAATACTGAATCTGATGGAAAAA
CTGAAGTTCCTGCTCCCCTTAACCAAGATACCACTTCAGGAGGAAAATTAACCTTTCCGC
TAGTTAGTATAGCAACAAGTCTATTGTTTGACAAAGAATTCATGAAACTAACAAAAGCAG
CTGATGTAGTATTATTTGAAGTAGAAAAAGCATATGACTTACTATCAACTAATGCTCCTG
TTGAAGGGTCAAAAACTCTTAAATGAAGACCTGCGCTAAATGTTTGATTTCCGACAGGGT
TATCCTGAAATTTAACATATGGTCCTGCTGTAGAAAGCGGGCAAGAACCCGCTATACTTA
TTTTGTATCTTACTGAATTACTTTCCGATAAAAATGGCGTTTGGACATATCCTTGTCCAA
ACTCTGCCATAAATTTTTCCATAATACCTCTTATTCAATCCACTCAAATTTAACAGTTTT
ATTCACTGGGTCAGGAATAATGCGAACATTACCAATTCGCAAGAAATCACGAATAGTAAG
ATTACCTATTATTCCATTATCAGATGGGATCGCTCCTATATCTGAAGGTTGAGGAGGGTT
ACCTCCGTCAAATACCTGAACAAAACTTGACCAAGAGTTTTTGGTTTTCTGCCATGTACG
TGTCCAGCGAGTGGTACGTGCTTCTGGAGTCGTTGGATAAGTAATCCAATCTTGGTAAAG
TGAATCAAGCGTGTTACCAAACTGAGTCAATGTACCAGGAGATTTAACTTCTTCGCCACG
TTCTAAGTATGGAAGCCCAGTCACTTCATTAGTTTTTTCAACCATCTTAAAATAACCCGG
GAACTGGTTATAAGTGGCTGAATCATTAATATCGATTGACCAGAATCCTACAGTATCAGA
TGTTGGCGCACGGGTATATAAATCAGAGGTTTTAGTACCCTGAGAACGAATTCTCGAGTT
AACAGTTAAACCACCTGAACTTATAGTAGCACCTTTGGCAATGATTAAGCTTTCACCAAT
CGTTACTTGACCAGATGCATTATTAATAGTTAAAGGACGTAATCCATTAAATCCACCGGT
CTGATCGCCAGATGCAGTAAGCATAAAATAGGTGTTAGCACCATCATTACGAATAAAGAA
TCCATAATCACCGTTTATTGCTCTAAAAGCATTCGATGATTTACTGATAAATTCGCCATT
AGCAGTAACTGAACTACCGAATGTTGCAACGCCATTCGCATTCAATGTTCCTGAAGCATT
CACATTTATCGGCGTTACTGTACCGTTTATATTAAATGTTATATTACCAGCTTTATTACG
CTGAGAATAAAAATGACTAGAAGTTTCATCACTAACTTCAAATACAGTAGAACGTGTTGT
GTCCGATTCACCGCTGAATTGATTTCCCCACACTCTGACTGTCATCATTTGAGCTGGGTT
CGTCCCAGTTTGAGGTCCTTTCTCAAAAATCAGACGAGTTGCCGTTCCAGTATTAGAAAT
AGTTAATGTACTATTTGCCGAAACTGATCCACCAAACGTAGCAGTACTAGATGATACAAG
AGGGGCACTCAGATTCGTTTGTTGCGTTAAGGTTAGTGAACCATTAACCGTCTGTGCAAT
ATCCCTACGAATGAACTGGAGTGAATCTAAACCATCTAATAAATTACTATCTACAGCTTT
TGCTTTTAACGGCAAATAATTTGCTAATACGCGGTTTAATTCATATGGTGATACTGCATA
GTTATTTTTCTCATATAGTTCTAAGTCCTGTGTAGAACCAGCTGTATCATTACCAACGAA
CGTAATTGAACCAGATGAGGTTTTAACAAAACCTCTTATCGCAGTAGTAGCTGCCCAAGT
AGGTTCACTCTGCACAATCCATTTTAAATTTTTTGGAGATACAGCAGTATTTGCTGACGT
TCCAGCTACAGTTTCAGACCGAGTTGCAACTTTAATAACGCCTTCCTGTGCTTCAGTAGA
TTTAGTACCTAAAAGCTTTTTAGGAGTTATTAGAACATTATCCAATGTTCCTGCAGCAGC
TTCAACTTGTGTAGCTACACGAAGTGTACCACGCTGTGTCTCATTTGCTTCAAGAATATT
AAGGGTATAATGGTCCCAGAGAGTTCCTGATTCAACTAATCCAGATAGAGCAACAACAGA
AGTACGATCAGTACTATTAAATCTGGTTTTAATTTTTAATGGTGTAGAGATACGAGTATC
GTCGACGCCTGCGTCGAATTCAACTTGTGTAGCAATTTCAGCTATACCAGTTAAACTTTC
AGTTGCTTTACGGTCATTTAAAGTTTTAGGAGTGACTGCACGAGTATAATCAGTTCCTGT
ATTAACTTCACTTTGTGTAGCAATTTCAATTAAACCAATTCTTGCATCAGTTGATGTCTT
TTTATGTAACGTTTCTGGCGTTACAACAGCATTTGCCCATCCTTTTTGACTCTGTCCAGC
AATTACTTCACTTTCAACTGCTAAAATTACTGCACCTTGTTGCGTTGGAGTAGCTTTATA
CTGATCCAAAGCTTTAGGCGAAACAACTAAATTATTAGTGTTTTTATTATAAACGTTTGT
ACCATTTAATTCACGACTAGAAGCTGGAGTAGCTCCTGCGGTAGATACAAATGTTACAAT
ACCGGATAATGATTCAGAACCTTGACGAGCTTGAAGCTTTTTAGGAGTGATGATTGTAGT
ATCATCAGTACCTGTATTAGTTTCCTGCTGCGTAGCAATCTCAGCGACACCTCTACGAGT
TTCTGTAGCAGTTCTTTCATTCAGCTTTTTAGGAGTGATGATAATATCATCAGCAAAAGA
GAATGTGGTGTTCTGATTCACTTGAGCAGTAGTTGCTATTCTTGCGATACCTCTACGAGT
TTCTGTAGCAGTACGATTAGCTAATGTTTCTGGTGTAATTGCTAATTCTTTTTGTGGAGA
ATTTTCTAAATCAGCATTTGCTTGAGCTTGTGTAGCTAAAGCAATTACGCCTAATCTTGC
TCTAGTAGAATTATTTAAAGAATCGACTCTTTCAACTGTTGGAACGTTTTGCTGTACAAC
CCAGTATTTTCCATCAGAATCTTCTATATAAGCAAGTTGCAAAACTGGTACATAATTAGT
TTCACCATTAAAAACTAATTCTTGAACTGTTACCCATTCAGCTTCAGGCGGATATTCTGA
ACGTTTTGGGAATTGCAGCAATTGAACTGAAGAAGCAATTTTATCTTCACCGGCAGCTTT
GATTTTAACTGTTTGTCCTTTTCTCATGTAATTCATGGAAATTTTAACAGTATCACCAAC
AGAAATATCAGTCGGAAGCTGAAGTTCAATTGTTTGGGTTGTTCCATTATTCGCACCAAA
TACCATGACTTCTTCATTTGGACGAATATTTGAATTAGTTGTTATAATGCGTAAACGTGC
TTTACTATCCCCGTCAAACAATCTCCACAATTTCTCATTATCGTCAAACATCAAGAAACC
GTCAATCGATGTACGGCCTTCAATGGAATGAGTTCCAACTTCTTGCACTGACGTCGTTTC
ATCATATGTCGTAACAATTGTATGATAAAGTGGATTTAATTTATCTAAATCAACGAAATT
AATGATATCTCCATGATTGGCAAATCTTGGAAGTTTAACATTAATCGGTGCGGCAGAAGT
AAATCTACGTACGATAAAATCATTAGATTGTGCTTGATATGCAGTCGATGGAGTAACAAT
TGCAGCTTCTCTGCTATAATCAGCAATATACATTTGCCACAAACGATTATTAAAAATGAA
TACCATCTGTGACTTTGGATGAGTCATTAAAACTGAACGTACCTGTTCACCTCTAAAATT
GACAATACTCTGATTTGAAGAATTTATTTTAACCTGATTTATGCCAGGTTTTCCACCGAT
ATCTTGAATTATTACGGTTTCTCCATCAAGCGGAGAAGGTGGTAAAGTAAACTCAATGTC
ATTGCCAACTGATGTATCTACTGAAATTGCTTCTCCGGATTTTAATTGATATGGTCCAGA
TGATACGGTTGTATATACGGCATCAGTACGTAATGCTTTCCAACGAATTCTATTAAAACT
TCCAGCAGGTTTTGGAATATTATCCGTTGCTGCCCAAAAACGATTATTATAAATGATTAC
AAAATCTTTTAAATATCCACGAGTTGGATCATATTGTTGAACTGTGTTTTCTTGAATTAA
GTAATCAACGTTAACACCGTCAGTTCCTACGGTACGATCAGCTAAAGCTACGTTGATTAT
TTTATCGCCACCTGCGTCCAGACCGTCTTCTGCTCTGAACTTTCTTTTAATCTCGGCCAT
TCTCCCGGGCTCCTATTGTGTTTTCAATAATAAGTATTTATACTTGTTTACTTTAAGATT
TGGATAGTATATAATAGAAATCTCACTAATTGAACGAGGTTCATATGGATTTAGAAATGA
TGCTGGATGAAGATTACAAAGAAGGAATTTGCTTAATTGACTTTAGTCAAATTGCGCTTT
CAACTGCTTTAGTAAACTTCCCAGATAAAGAAAAAATTAATTTATCAATGGTTCGTCATT
TGATATTGAACTCAATTAAGTTTAATGTCAAAAAAGCGAAAACTCTTGGATACACTAAAA
TTGTACTGTGTATCGATAACGCGAAATCTGGATACTGGCGTCGTGATTTCGCTTATTACT
ATAAGAAAAACCGTGGAAAAGCACGAGAAGAATCTACTTGGGATTGGGAAGGTTATTTTG
AATCCAGCCATAAAGTTATAGATGAATTGAAAGCTTATATGCCATACATTGTTATGGATA
TTGATAAGTATGAAGCAGATGACCACATTGCTGTTCTTGTTAAAAAGTTCTCTTTAGAAG
GACATAAGATTTTAATCATTTCATCGGACGGTGACTTTACTCAACTTCACAAATATCCAA
ATGTTAAGCAATGGTCACCGATGCACAAGAAATGGGTTAAAATTAAAAGCGGTTCTGCTG
AAATTGATTGTATGACTAAAATTCTTAAGGGTGACAAAAAGGATAACGTTGCTTCAGTTA
AAGTACGTTCTGACTTTTGGTTTACCAGAGTTGAAGGTGAACGAACTCCTTCAATGAAAA
CTTCAATCGTTGAAGCTATTGCTAATGACCGTGAGCAAGCTAAGGTGCTTCTCACAGAAT
CTGAATATAATCGTTATAAAGAAAATTTAGTTCTAATTGATTTTGATTATATTCCTGATA
ATATTGCTTCAAACATTGTGAATTACTATAATTCATATAAATTACCACCGCGTGGCAAAA
TTTATTCATATTTTGTAAAAGCGGGTCTTTCTAAATTAACTAATAGCATTAATGAATTTT
GAGGTGAATAATGGCTAAAAAAGAAATGGTTGAATTTGATGAAGCTATCCATGGCGAAGA
CTTGGCTAAATTTATTAAAGAAGCATCTGATCATAAACTGAAAATTTCCGGTTATAATGA
ACTGATTAAAGATATTCGAATTCGTGCCAAAGATGAACTTGGCGTTGATGGTAAGATGTT
TAATCGTCTATTAGCTTTGTATCATAAAGATAACCGTGATGTGTTTGAAGCTGAAACTGA
AGAGGTAGTTGAACTTTATGACACAGTTTTCTCTAAATGATATTCGTCCGGTCGATGAGA
CCGGTCTTTCAGAAAAAGAACTTTCAATCAAGAAAGAAAAGGATGAAATAGCAAAGCTTC
TTGATCGTCAAGAAAATGGATTTATTATTGAAAAAATGGTAGAAGAATTTGGAATGAGTT
ATCTTGAAGCTACAACAGCATTTTTGGAAGAAAACTCTATTCCTGAAACTCAATTTGCTA
AATTTATTCCTTCGGGTATAATTGAAAAAATTCAGTCAGAAGCCATTGACGAAAATCTTT
TACGTCCTTCTGTTGTTCGTTGTGAAAAAACTAATACATTAGATTTTCTACTATGATTAA
ACTCCGCATGCCTGCTGATGGTGAAAGATACATTGATGGTAAATCAGTTTATAAATTATA
CTTAATGATAAAACAACATATGAATGGAAAGTATGATGTTATTAAGTATAATTGGTGCAT
GCGGGTGTCTGATGCCGCTTATCAAAAGCGAAGGGATAAGTATTTTTTCCAAAAGCTATC
AGAAAAGTATAAATTAAAAGAATTAACTTTAATTTTTATTAGCAATCTTGTAGCTAACCA
AGATGCTTGGATTGGTGATATCTCTGACGCTGATGCATTAGTATTTTATCGTGAATATAT
CGGACGCTTAAAGCAAATTAAATTTAAGTTTGAAGAAGATATTAGGAATATCTATTATTT
TAGTAAAAAAGTTGAAGTTTCTGCTTTTAAAGAAATCTTTGAGTATAATCCAAAAGTTCA
GTCGAGTTATATTTTTAAACTACTTCAATCGAATATAATTTCGTTTGAGACGTTTATCTT
GCTTGATTCGTTTTTAAATATAATCGATAAACATGATGAACAGACTGATAATTTAGTTTG
GAACAATTATTCTATAAAGTTAAAAGCTTATAGAAAAATTTTAAATATTGATTCACAGAA
AGCTAAAAATGTTTTCATTGAAACTGTGAAATCTTGCAAGTATTAATTGCTTATTATAAA
TAGATTATAATTATCTCACTGACCAGCTATGAGGTCATACATCGTCATAGCACCAACTGT
TAATTAAATTAAAAAGGAAATAAAAAATGTTTAAACGTAAATCTACTGCTGAACTCGCTG
CACAAATGGCTAAACTGGCTGGAAATAAAGGTGGTTTTTCTTCTGAAGATAAAGGCGAGT
GGAAACTGAAACTCGATAATGCGGGTAACGGTCAAGCAGTAATTCGTTTTCTTCCGTCTA
AAAATGATGAACAAGCACCATTCGCAATTCTTGTAAATCACGGTTTCAAGAAAAACGGTA
AATGGTATATCGAAAATTGCTCATCTACCCACGGTGATTACGATTCTTGCCCAGTATGTC
AGTACATCAGTAAAAATGATCTGTACAACACTGACAATAAAGAGTACAGTCTTGTTAAAC
GTAAAACTTCTTACTGGGCTAACATTCTTGTTGTAAAAGACCCAGCTGCTCCAGAAAACG
AAGGTAAAGTATTTAAATATCGTTTCGGTAAGAAAATCTGGGATAAAATCAATGCAATGA
TTGCAGTTGATGTTGAAATGGGTGAAACTCCGGTTGATGTAACTTGCCCGTGGGAAGGTG
CTAACTTTGTACTGAAAGTTAAACAAGTTTCTGGTTTTAGTAACTACGACGAATCTAAAT
TCCTGAATCAATCTGCGATTCCAAACATTGACGATGAATCTTTCCAGAAAGAACTGTTCG
AACAAATGGTTGACCTTTCTGAAATGACTTCTAAAGATAAATTCAAATCGTTTGAAGAAC
TGAACACTAAGTTCAGTCAAGTTATGGGAACTGCTGCTATGGGTGGTGCCGCAGCAACTG
CCGCTAAGAAAGCTGATAAAGTTGCTGATGATTTGGATGCATTCAATGTTGATGACTTCA
AAACAAAAACTGAAGATGATTTTATGAGCTCAAGCTCTGGCAGTTCATCTAGTGCTGATG
ACACGGACCTGGATGACCTTTTGAATGACCTTTAATAGATTATATTACTAATTAATTGGG
GACCCTAGAGGTCCCCTTTTTTTATTTCAAAAATTTTTTCACAAAATGGTTTACATCCCT
GTTCTTCTATGGTACTATACAACTATCGGCAATACTGCTGACGATTAAAGAGGAAAACAA
TATGGCTAAAGTTGATATTGACATCGTTGATTTTGAATATATTGAAGAGATTATTCGTAA
TCGTTATCCTGAACTTAGTATCACAAGCGTGCAAGATTCTAAGTTTTGGAGTATTCAAAT
CGTTATTGAAGGTCCTCTTGAAGACCTCACCCGCTTTATGGCTAATGAATACTGTGATGG
CATGGATTCTGAAGACGCAGAATTTTACATGGGACTGATTGAACAATAATTATCGAGGGG
CTATCAAGCCCCGTTAAAATGAGGAAACCGTAATGTATACCGGTAAAAAGTATGAACTTG
TTCCAAGACTTATTGATACATTTATTAATTATAGCCCACGTTCTAATTCATCAATAGTTA
AAGCCATTCGAGAGAATGGCGGATGGTTTGAAGTTAAAGAAGTTTCCGTCGCTAATGGAT
ATAAAGCAGTAAAACACATTGAATGCGCAAATGGAAAACATTTTTACCTCAAAGATAGAC
TTTTAGAAATTTGCGAAGACGAATTTCATTGTTTTCGTGAGTATAAAGAACCGACTTCTG
AAAAAGATGAAGTCAAAGACAAGGTTTATGGCGTAACAAAAATTCACTGCATTGTAGACG
AAAACAATGTAGATGAAATCATTGAACTTTTGCGAAAAACTTTCAAAAAGTAGTTTACAA
CAGGGTAGTAGTGTGATACTATTACCCTATCAATTAAGGAGAATAAAATGAGATTACAAC
GCCAAAGCATCAAAGATTCAGAAGTTAGAGGTAAATGGTATTTTAATATCATCGGTAAAG
ATTCTGAACTTGTTGAAAAAGCTGAACATCTTTTACGTGATATGGGATGGGAAGATGAAT
GCGATGGATGTCCTCTTTATGAGGATGGAGAAAGCGCAGGATTCTGGATTTACCATTCTG
ACGTCGAGCAGTTTAAAGCTGATTGGAAAATTGTGAAAAAGTCTGTTTAAGGAAAATAAT
ATGATTTTTGTATTTGAATTTATGAATGATGAATTCGATTATGCAATTTTTAACGCATTG
CATAATCCTGATTTAAGTGAATTTAATGAAATGTTTTCTGACGCTTTGAGTATGTCAGAA
GAATACTGTGGAGAATGTCAACGTGTTTGTGTGACAGTTTTTGAAAACAAAGAAAAGACA
TATGAAGAATTATTCTTTGATGCTAATAAAGCCACTGAATGGTTTCTTGAAAGGGGTTTT
GCGTAATGATTAAATTGGTATTCGCTTATTCTCCAACTAAAACGGTCGAAGGCTTTAATG
AATTAGCATTCGGTTTAGGTGATGGTTTACCATGGGGGCGAGTTAAAAAGGACCTCCAGA
ATTTTAAAGCTCGTACCGAAGGTACAATTATGATTATGGGTGCTAAAACGTTCCAGTCAT
TATCTACATTGCTTCCCGGTCGTAGTCATATTGTGGTGTGTGACCTTGCGCGTGATTATC
CTGTAACTAAAGACGGCGATTTAGCACATTTCTATATTACTTGGGAGCAGTATATAACTT
ACATTTCTGGCGGCGAAATTCAAGTGTTAAGCCCTAATGCACCATTTGAGACTATGCTTG
ATCAGAATTCCAAAGTAAGTGTAATTGGCGGGCCTGCTCTGTTATATGCTGCATTACCTT
ATGCGGATGAAGTAGTTGTTTCTCGCATCGTTAAAAGGCACCGTGTTAATTCAACAGTTC
AATTAGATGCAACTTTTCTTGATGATATAAGCAAGCGTGAAATGGTTGAAACGCATTGGT
ATAAAATAGATGAAGTAACAACTCTTACGGAATCAGTATATAAAAATGAAATACCGCGGG
GTGGAAAATATGAATTTTAATTATTACCCTATTCTATTAGAAAAAGACACGAAACAGCCA
AAATGGCAAGGTCCTCAGTTTATTAAAGGTGTCTATCAATTAGTAGTTCCTAAAGACAAG
ATTTATAGCAGTTGTTTCACTGAATCCGCTTGTAGTATTTTTGGTAATAGTTCTCCGTAT
TGGAATTTTGACATAAAGCTGGATAGAAATATCGATATTTGGTTGAAAGCTATGGATATT
GGTAATATCACGTTTGATGAGAATAATTATCATATTATTGGTCGCTTTTCCAAACGTGGC
AAAGAATTATACTTCACGCCTGAAATTGAGAGAAAATTTGATGCTAAACCGTATTGATTA
TTAGCTTCATAAAATGTATTATAATTTATATAAGGTACTAAATGAAACAATACCAAGATT
TAATTAAAGACATTTTTGAAAATGGTTATGAAACCGATGACCGAACAGGCACAGGAACAA
TTGCTTTGTTCGGTACTAAATTGCGTTGGGATTTAACCAAAGGTTTTCCTGCAGTAACAA
CTAAAAAGCTCGCTTGGAAAGCTTGCATTGCTGAGTTAATATGGTTTTTATCAGGAAGCA
CAAATGTCAATGATTTACGATTAATTCAGCATGATTCATTAATTCAAGGCAAAACAGTCT
GGGATGAAAATTACGAAAATCAAGCAAAAGATTTAGGATACCATAGCGGTGAACTTGGTC
CAATTTATGGAAAACAGTGGCGTGATTTTGGCGGTGTAGACCAAATTGTAGAAGTTATTG
ATCGTATTAAAAAATTACCAACTGATCGACGTCAAATCGTTTCAGCATGGAATCCAGCTG
AAATCAAGCAGATGGCATTACCGCCTTGTCATATGTTCTATCAGTTTAATGTGCGTAATG
GCTATTTGGATTTGCAGTGGTATCAACGATCAGTAGATGTTTTCTTGGGTTAATTGAGGC
CTGAGTATAAGGTGACTTATACTTGTAATCTATCTAAACGGGGAACCTCTCTAGTAGACA
ATCCCGTGCTAAATTATAGGACTGCCCTTTAATAAATACTTCTATATTTAAAGAGGTATT
TATGAAAAGCGGAATTTATCAGATTAAAAATACTTTAAACAATAAAGTATATGTAGGAAG
TGCTAAAGATTTTGAAAAGAGATGGAAGAGGCATTTTAAAGATTTAGAAAAAGGATGCCA
TTCTTCTATAAAACTTCAGAGGTCTTTTAACAAACATGGTAATGTGTTTGAATGTTCTAT
TTTGGAAGAAATTCCATATGAGAAAGATTTGATTATTGAACGAGAAAATTTTTGGATTAA
AGAGCTTAATTCTAAAATTAATGGATACAATATTGCTGATGCAACGTTTGGTGATACATG
TTCTACACATCCATTAAAAGAAGAAATTATTAAGAAACGTTCTGAAACTGTTAAAGCTAA
GATGCTTAAACTTGGGCCTGATGGTCGGAAAGCTCTTTACAGTAAACCCGGAAGTAAAAA
CGGGCGTTGGAATCCAGAAACCCATAAGTTTTGTAAGTGCGGTGTTCGCATACAAACTTC
TGCTTATACTTGTAGTAAATGCAGAAATCGTTCAGGTGAAAATAATTCATTCTTTAATCA
TAAGCATTCAGACATAACTAAATCTAAAATATCAGAAAAGATGAAAGGTAAAAAGCCTAG
TAATATTAAAAAGATTTCATGTGATGGGGTTATTTTTGATTGTGCAGCAGATGCAGCTAG
ACATTTTAAAATTTCGTCTGGATTAGTTACTTATCGTGTAAAATCTGATAAATGGAATTG
GTTCTACATAAATGCCTAACGACTATCCCTTTGGGGAGTAGGGTCAAGTGACTCGAAACG
ATAGACAACTTGCTTTAACAAGTTGGAGATATAGTCTGAACTGCATGGTGACATGCAGCT
GGATATAATTCCGGGGTAAGATTAACGACCTTATCTGAACATAATGCTACCATTTAATAT
TGCATCATATGCTGCGTTAGTTCATATTGTAGCTAAGATGTGTAATCTTATTCCTGGAGA
TTTGATATTTTCTGGCGGTAATACTCATATCTATATGAATCACGTAGAACAATGTAAAGA
AATTTTGCGTCGTGAACCTAAAGAGCTTTGTGAACTGGTAATAGGCGGATTGCCTTATAA
ATTCCGCTATCTTTCTACTAAAGAACAATTGGAATACATTCTTAAACTCAGGCCTAAAGA
TTTCGTTCTCAAAGATTATCAGTCCCACGGTGTCTTGAAAGGAAAAATGGCGGTATAATT
TTAATTTAATTGCGAGGATATATGATTTTACGATTTAAAGATACTTCTGACGTTGTTCTT
TTTACACTTCCTAATCCAAGTGAGCTAGAAGTTCCAGGACCAAATCAGCCTATTATCATT
TATGGCAAAAAATATTATACTCATAAAATGACTCGTGAGTATTTTGATAATAAAATTTCC
ACAGTTAAAACTTCTTCTGACTGTTACTACGATATTACTGTTTTAACGGAAAAACAATAT
GCCGAATTATCACCGCGCGGGCCGTCTATGTCGGGTAGTGAATAAATACAAATCCGACTT
TGATGTTAATATTCACCGTGGTACATTTTGGGGAAATTACGTCGGTAAAGATGCTGGCAG
CCGGGAGGTTGCCATTGAATTATTCAAAAAAGATTTTATACGTCGAATTAAATCCGGAGA
AATAACTAAAGCACATTTAGAGCCTTTACGTGGAATGAGGCTAGGATGCACATGTAAACC
AAAGCCGTGTCATGGTGATATAATAGCTCATATAGTTAACCGATTGTTTAAAGACGATTT
TCAAGTTGAGGACTTATGCAATTAATTAATGTTATCAAAAGTAGTGGTGTTTCTCAGAGC
TTTGACCCACAAAAAATTATTAAAGTTTTATCTTGGGCAGCTGAGGGAACATCAGTAGAT
CCTTATGAATTATATGAAAATATTAAATCTTATCTCCGTGATGGAATGACAACTGATGAT
ATTCAGACTATTGTCATTAAGGCTGCTGCGAATTCTATTTCGGTTGAAGAGCCTGATTAT
CAATATGTAGCTGCACGTTGTCTAATGTTCGCTCTTCGTAAGCATGTTTATGGGCAGTAT
GAACCGCGTTCATTTATTGACCATATTTCTTACTGTGTAAATGAAGGTAAATACGACCCT
GAATTGTTGTCAAAATATTCTGCAGAAGAAATTACATTTTTAGAATCAAAAATTAAGCAC
GAGCGGGATATGGAATTTACTTATTCCGGGGCGATGCAATTAAAAGAAAAATATCTCGTT
AAAGATAAAACCACTGGTCAAATTTATGAAACTCCACAGTTTGCATTTATGACTATTGGA
ATGGCATTGCATCAAGATGAACCTGTTGATAGACTAAAACACGTTATCCGTTTTTATGAA
GCAGTATCCACTCGACAGATTTCATTGCCAACTCCTATTATGGCTGGTTGCCGTACTCCA
ACTCGGCAGTTTAGTTCATGCGTTGTTATTGAGGCAGGTGATTCATTAAAGTCTATTAAT
AAGGCTTCTGCTTCAATTGTTGAATATATCTCCAAACGTGCTGGAATTGGTATTAACGTT
GGTATGATTCGTGCCGAAGGTTCTAAGATTGGCATGGGTGAAGTGCGTCATACTGGTGTT
ATTCCTTTTTGGAAACATTTTCAGACTGCTGTTAAATCATGCTCACAGGGCGGAATTCGT
GGCGGCGCTGCTACTGCTTATTATCCTATTTGGCATTTGGAAGTTGAAAATCTTCTCGTT
TTGAAAAATAACAAAGGCGTAGAAGAAAACCGCATTCGTCATATGGATTATGGTGTTCAA
CTGAATGATTTGATGATGGAACGATTCGGAAAGAACGATTACATTACTTTGTTCAGTCCA
CATGAAATGGGTGGCGAGCTTTATTATTCTTATTTTAAAGACCAAAACCGTTTCCGTGAA
TTATACGAAGCAGCAGAAAAAGACCCTAATATTCGTAAAAAGCGTATTAAAGCCCGTGAA
CTATTTGAATTGCTCATGACTGAACGTTCAGGAACCGCAAGGATTTATGTGCAGTTCATT
GATAATACGAATAACTATACTCCGTTTATTCGTGAAAAGGCGCCTATTCGTCAGAGTAAC
TTGTGCTGTGAAATTGCTATTCCAACAAATGATGTGAATAGTCCTGATGCTGAAATTGGC
TTGTGCACGTTATCTGCATTCGTACTAGATAATTTTGACTGGCAAGACCAAGATAAAATT
AATGAATTGGCAGAAGTTCAAGTTCGTGCTCTTGATAATCTGTTGGATTACCAAGGATAT
CCAGTTCCTGAAGCAGAAAAAGCTAAAAAGCGTCGTAACCTTGGCGTAGGTGTTACCAAC
TATGCAGCTTGGCTGGCAAGTAACTTTGCTTCTTATGAAGATGCTAACGATTTAACACAT
GAACTATTTGAGAGATTACAGTATGGGCTCATTAAAGCATCCATTAAGCTCGCCAAAGAA
AAAGGACCTTGCGAATATTATTCAGACACTCGTTGGTCTCGAGGCGAATTACCTATCGAC
TGGTACAATAAAAAGATTGACCAAATCGCAGCTCCAAAATACGTTTGTGACTGGTCGTCG
CTGCGGGAAGACCTTAAGCTCTTTGGCATCCGTAATAGCACATTATCAGCACTTATGCCA
TGTGAGTCATCTTCCCAAGTTTCTAACAGTACAAACGGTATCGAGCCTCCACGTGGACCA
GTCTCTGTTAAAGAATCAAAAGAGGGCTCCTTTAATCAAGTCGTGCCCAATATTGAGCAT
AACATAGACCTCTATGATTATACATGGAAATTAGCTAAGAAAGGTAATAAACCTTATCTT
ACGCAGGTAGCTATTATGCTGAAATGGGTATGTCAATCAGCTTCAGCGAATACATACTAT
GACCCGCAGATTTTTCCAAAAGGAAAGGTTCCAATGTCAATAATGATTGATGACATGCTA
TACGGATGGTATTATGGCATTAAAAATTTCTATTATCATAATACTCGTGATGGTTCTGGT
ACTGATGATTATGAAATAGAAACTCCAAAAGCTGAAGATTGTTCATCCTGTAAATTATGA
TATAATTTGACTCACGGACGAGTCACCAACTATTAACTAAGTGGAAAATTTATGAGCACA
GTTTTTAATACAAACCCAGTTGATGTTTTAAAAGAACCTATGTTTTTTGGTTCAGGTCTT
GGTATTGCGCGTTATGATATTCAACGGCATCGAGTATTTGAAGAACTTACAGAAAAGTCC
TTATCATTTTTCTGGCGCCCGGAAGAAGTAAACTTAATGATGGATGCTGCACAGTTTAAT
AAGCTTCCTCAGTATCAGCAAAATATTTTTACTAACAACTTGAAGTATCAATCACTTTTG
GATAGCATTCAGGGTCGTGCGCCGTCTGCTGTACTTATGTCATTAATTTCAGACCCAAGC
CTTGATACGTGGGTTGCTACATGGACTTTTAGTGAAACTATTCACAGTCGTTCATATACA
CATATCATGCGAAATCTTTATACTGATCCATCGAAGGTATTTGATGAGATTGTATTAGAT
GAAGCTATTATGAAACGTGCTGAATCCATTGGACGTTATTATGATGATGTTCTGATTAAA
ACTCGTGAATGGGAAAATGCTAAAGAATTTGTAGAACTATCTAAAGAATCTCCTGATGCC
GACTTTCGTTTAAACCGAGCTATTAAACAAGAAGCCGAAGCTAAACGTGCTTTAATGAAA
TCTCTTTACCTCTGTTTGCACGTTATTAATGCCTTAGAAGCTATTCGTTTTTATGTATCT
TTCGCATGCACTTTTAACTTCCATAAGAACATGGAAATCATGGAAGGTAATGCCAAGATT
ATGAAGTTCATTGCGCGTGATGAGCAGCTTCACCTTAAAGGCACTCAATATATTATTCGT
CAACTTCAACTTGGCACTGATGGCGATGAATGGGTTAAAATTGCTCAAGAGTGTGAACAA
GAAGCAGTTGATATTTTCATGGAAGTTAACCGCCAAGAAAAAGATTGGGCAGTTCATTTA
TTTAAAGATGGTGATGTTCCTGGATTAAATACAAATAGCATGTGGAGCTTTATTGATTAC
TTAACTGTATCTCGTATGAAACAGTGTGGTCTTCCATGCCCAATTACCGATGCTCCAATT
AAACATCCATATCCTTGGATTCGTGAATATCTTAATTCTGATAATGTTCAATCCGCGCCA
CAAGAAGTAGAACTGTCATCTTACCTTGTTGCACAGATTGATAATGATGTTGATGATAAA
GTTATGATGAGTTTTAAAAAATACTTTTAAGGAGTGGGCCGCAAGGCCCATTTTATTATG
AAAGAAATTGCAACAGAATATTCATTTATTAAATATACTGAGCTAGAATTAGACGACAAT
GGAAGTATAAAACAGTTATCTATTCCAAACAAGTATAATGTAATTTATGCTATTGCTATA
AATGATGAGCTTGTTTATATTGGAAAAACTAAAAATTTACGCAAAAGAATAAACTATTAT
AGAACTGCTATTAACCGTAAAGACAAAACGTCTGATTCTACTAAATCTGCATTAATTCAT
GCTGCGCTAAAGGAAGGAAGCAAAGTTGAATTTTACGCCCGCCAATGTTTTAATCTTTCT
ATGACAAATGAGTTAGGTACAATGACAATCGCAACGATTGACTTAGAGGAGCCACTATTC
ATTAAACTGTTTAACCCGCCTTGGAATATTCAACATAAGAAAAAATGATGCTTCCACATG
GAGTGTGGTACTATATTCAAAACACAAAAGAGGATACACAATGCAAGAACTTTTTAACAA
TTTAATGGAACTATGTAAGGATTCACAGCGTAAGTTTTTTTACTCAGATGATGTAAGTGC
ATCTGGAAGAACTTACAGAATTTTCTCATATAATTATGCATCTTATTCTGATTGGTTACT
TCCAGACGCATTGGAATGTCGTGGAATTATGTTTGAAATGGATGGAGAAAAACCGGTAAG
AATTGCCTCTCGTCCCATGGAAAAGTTTTTTAACTTGAATGAAAATCCATTCACGATGAA
TATCGATTTAAATGATGTTGATTACATTCTAACAAAAGAAGATGGGTCTTTGGTATCAAC
TTATTTAGACGGTGATGAAATTCTGTTCAAATCAAAGGGTTCAATCAAATCTGAACAGGC
TTTAATGGCTAATGGGATTTTGATGAATATTAATCATCATCAGTTGCGCGACAGACTTAA
AGAATTAGCCGAAGATGGATTTACTGCTAACTTCGAATTCGTCGCTCCAACTAATAGAAT
CGTTCTTGCTTATCAAGAGATGAAAATCATTTTATTGAATGTTCGTGAAAACGAAACGGG
TGAATACATTTCATATGATGATATTTATAAAGATGCTATTCTTCGTCCGTATCTAGTTGA
ACGATACGAAATCGATAGCCCTAAATGGGTAGAAGAAGCTAAAAATGCAGAAAACATCGA
AGGCTATGTTGCTGTGATGAAAGATGGTTCTCATTTTAAAATTAAGTCTGACTGGTATGT
GTCTCTTCATAGCACAAAAAGTTCATTAGATAATCCAGAAAAATTGTTTAAGACTATTAT
TGACGGTGCATCAGATGATCTTAAAGCAATGTACGCCGATGATGAATATTCATATAGAAA
AATTGAAGCATTTGAAACGACTTATCTGAAGTACTTAGACCGAGCTCTATTCTTAGTTCT
TGACTGTCATAATAAGCATTGTGGTAAAGATAGAAAGACTTATGCAATGGAAGCGCAAGG
CGTTGCTAAAGGTGCTGGAATGGATCACCTGTTCGGTATCATCATGAGCCTATACCAAGG
GTATGATAGTCAAGAGAAGGTCATGTGTGAAATCGAACAGAATTTTTTGAAAAATTATAA
AAAATTTATCCCAGAAGGATACTAAGCTGTTTACAAGTCCCTCGTGTTGTGTTATAGTAG
TCTTACTGACATAACATGAGGACTTTATGATGGATTTGCAGCTTATTACTACTGAAATGG
TCGTTGAAGCATACGGTGATACTACAGATGGGATTTCTGTATTCAAAGGAAATCGTCGAG
TTGGATATATCACCGATCTTAAGAAAGATTTAGCTAAGCAAGTCAAGCGGAAAACGACCA
TTAAAGAATATCGAAATCGTCGTCTTGAGCAAGCCCGTGATATGCTTCCTGATGCGGTTG
AAGAGATGAAAGTCTTTTTAGAAAATCAACTCGCTAAGTATGATTGTGAAGTGTTCATTA
ATCAGACTCAGCCTAATGTTCATATCAATAGCTGCAAATGCTATATCATCGTTAATCCTT
TGACTGGAAAGCATCGTCTTGGAATTAGTAATCCAAATCGTAGTGCATCGGATATGGCAG
AAGATGTTGAGGCATGCTTTAAAATTTCTAAATCTCCGGCTGAACATCATATTTTAATTA
ACGGTCTTTCTCAAGACGATATTATAGAGGTTATTAAAACTTTATGCAATTAAGTAATAC
AACAGCTGGATTGCTATTACTTGTAATAGCATTTGGCGGAACATCTATTATTTTAAAAAA
TAAGGTAGAAAGATTAGAAACATCAGTTACTGAAATTACAAAAACAGCCAATGAAAACGC
TTTAGCATTAGATAATTTGCGAATTCAGTATAATTATATTGATGCGATGAATAATAAAAA
TCGTGAGGCAATTGCTGCTATTGAGCGTGAAAATGAAAAACTGCGCAAAGATGCAAAGAA
GGCGGATGTGGTGGCTCATAAGCCAGGATTGGTTGAAAAACAAATCAACAACTCCTTCAA
CAAGTTCGCAGAAGACATCCAGGACCTTTCTAAATGATTAAACTATCAGCAGTAATATTA
TCTATTGGTCTTCTAGTTGGTTGTTCTACAAAGCCTCTAGAAGTAAAGAAAGAAACAGTT
CATCCTAATTGGCCTGTACAAATAAAGTCATATGATGAAGCTAAACTATCTTGGCAAGTT
AAAGTTATTGATGGTAAAGCTTGGGTCGGTATGCCATTTGAAGATTCCCAGGAATTTCGT
ATTTGGCTTAATGATGTAAAACGATATGTACATGACCAAAAAACTATGATATGTTATTAT
CGTCAAGAGCTAAAAGAGGATAAATGTAAATGATTACATGGCATCAATTTGAACATCTAA
AGGGATTAATTTATGAATCCGAGATGGCTGCAATGATTTATGGACGACAGATTCAACGAT
TAGAATCTTTACCTCCAACTAATGATGTTTTATTAGCTCAATCACGTGCTAATCTTAAAA
ATGAATACCAAAATAAGTGGGGTAAAGCATCTAAAGACCTGCATGATTATATTCAATCAT
TGATTGAGAAAAATAAATGAAAACTCTACTAGAACGTTATATTGAATGCTCAGACCGTTA
CATTGACGTATGCCGTGAAAATGTATCAAGCATTAGCGAAGACATTGAACATGCTAAAGC
TTTAGATGATGCTGGTAAAGCCCTGCGAAAAGAAGCGAAAGCTCGTGGGCTTGATATGTA
TCAGCTTAAAAATCACATGATAAAATTTATTTCATCTAATGTTCAAAGTAAATCAGTGAA
TCAATCAACAGCTGAATTGTACAAAGGACGGCGTGAGCATAATATTCGTATTCTTGAAGT
TTTCTTAGGAATTAAATGATGAAAAAGATTATTTTGACTATTGGCTGTCCTGGTTCAGGT
AAGAGTACTTGGGCTCGTGAATTTATTGCTAAGAATCCCGGGTTTTATAATATCAATCGT
GATGATTATCGTCAATCCATCATGGGTCATGAAGAACGCGATGAGTATAAGTATACCAAA
AAGAAAGAAGGCATCGTAACTGGTATGCAGTTTGATACAGCTAAAAGTATTCTGTACGGT
GGTGATTCTGTTAAGGGCGTAATCATTTCAGATACTAACTTGAATCCTGAACGTCGCCTA
GCATGGGAAACTTTTGCCAAAGAATACGGCTGGAAAGTTGAACATAAAGTGTTTGATGTT
CCTTGGACTGAATTGGTTAAACGTAACTCAAAACGCGGAACTAAAGCAGTACCAATTGAT
GTTTTACGCTCAATGTATAAAAGCATGCGAGAGTATCTCGGTCTTCCGGTATATAAAGGG
ACTCCTGGTAAACCAAAAGCAGTTATTTTTGATGTTGACGGCACGTTGGCAAAAATGAAT
TGTCGTGGTCCTTATGACCTTGAAAAATGCGATACCGATATTATTAATCCAATGGTCGTT
GAACTATCCAAGATGTATGCCCGTATGGGTTATCAGATTATCGTAGTAAGTGGTCGTGAA
TGTGGAACTAAAGAAGACCCAACGAAATATTATCGTATGACCCGTAAATGGGTTGAGGAC
ATTGCTGGCGTTCCATTAGTTATGCAATGTCAACGCGAACAAGGTGATACTCGTAAAGAC
GATGTAGTTAAAGAAGAAATTTTCTGGAAACACATCGCACCACATTTTGACGTGAAATTA
GCTATTGATGACCGAACTCAAGTAGTTGAAATGTGGCGCCGCATCGGTGTTGAATGCTGG
CAAGTCGCCTCGGGAGATTTTTAATGGCTTTGCACCATGAAACTTGGGATATTATTAATT
GGCTTGAAGAAAATTATGTGAGGTAAATATGTTTCCGACTTACTCTAAAATCGTAGAAGT
AGTGTTTAGCCAAATTATCGCTAATAATATGTTTGAAAAACTTGATAACGCAGCTGAACT
TCGAATCCACGCTCAAGTGACTCATGTATTGAACACTTTGCTTCCAGACCGGGTGGATTC
TGTTGTTATTCAACTGTATCCTGGGTCAGCGCATATCATTGTCGTATTTGGTCTTGATGC
TGAACTAGTTATTCAAGGCGACATTCGCTTTAAATCTCAAACCGCGGAATTCAAAGCGAT
TTAATAGTTTACTTTACGGTAGAGTTGTGATATTATAGCTCTACCAAAACAAATGAGGAA
ATTGAAATGAGCGAATGGTTTGAAGAAGATAAGGTTTATCGCTTTAAAACTGGGTATAAA
GATATTTTTAATGAAACTTGCGGGGCTAATAAACGAATTGCCCAGTTTATTGATGAAAAT
TCATTTAAAGTAAAAATTGACCCTGCAAAGAATGTTATTAGCATTAAACGCGAAATTGAT
GACTGCTGGTATAAAGCTATTGATGTAATGGGCGAATCCTATAAAGATAGCCCGTTATTT
TCAATTGCTTATATGTTAGAATATTCTTTTTTCGAAGAAGTTCAAAAAGATGATTCTGTC
AGTAAATTTGAAATTAAAACTGATAAAGAAATTAAGTGGAAAGTAGTAGGTATTACTGGT
TGTATGTTCTATATCTATGCTCAAACTGATACGAAGGAAGAAGCTAAAAAGAAAGCTCTA
GAATATCTTGAAGAGTATGAAGAAGGCCCGGTAATGATTACTCAAGATGCTGAATTAGTT
TCTGTCAAATTAGTTAAAAATGTTGAAAGTAAGGAGCTGGGATCAACATGCTAAGTGAAA
AACCAATTACTGTTAAAGAATTCCAAGAAAAAGTTAAAATATTTGCGCAGGAATTAGTAA
ATAAGGTTTCTGAACGATTTCCTGAAACATCGGTTCGTGTTATTACCGAAACTCCTCGTT
CAGTATTAGTAATTGTGAATCCAGGCGATGGCGATCAAATATCGCATCTTAAACTGGATT
TTGATGGATTAGTTGAAGCACAAAGGGTGTATGGCGTACTATGATGAATTTAACTGATAT
AATTGATAATTGTCTTGAAAATGATACTGGTGATCATAGAGCGCTTGATTCTGAAACAGC
AAAGTTCATTAGAATAACTTTAATGAATGATACTCTGGTGAATAGTATTCATCCTTCTGT
GTATGATGCTATTATTGTGACGAAGTATCCAGTTGAGCTTCACAAAAAGATGACTGGCGC
AGTTTTTATTGATAAGAAAAACCGCTTTAAAGATGGGCAGAATATAATTAGTTCTGTTAT
TAAAAGTATAACTAAACTTCGTCACGAAATTTATCGTGTTGAAACTGCTAAATCTGCTTA
TCTGGTGATTATGAAATGAAAGCGAGTACAGTACTTCAAATTGCATATTTAGTATCGCAG
GAATCAAAATGTTGCTCCTGGAAGGTAGGAGCAGTAATTGAAAAGAATGGACGTATTATT
TCTACTGGGTATAATGGTTCACCCGCAGGGGGTGTGAACTGTTGTGATTATGCTGCTGAG
CAAGGTTGGTTGTTGAATAAGCCTAAACATACTATCATTAAAGGTCATAAGCCTGAATGC
GTATCATTTGGTTCAACTGATCGTTTTGTCTTGGCGAAAGAACACCGCAGCGCTCACTCT
GAATGGTCGTCTAAAAATGAAATTCATGCTGAACTAAATGCAATTTTGTTTGCTGCACGA
AATGGTTCTTCTATTGAAGGTGCTACTATGTATGTAACACTTTCTCCTTGTCCAGATTGC
GCAAAAGCGATAGCTCAATCTGGTATTAAAAAGCTGGTTTATTGCGAAACATATGATAAA
AATAAACCTGGCTGGGATGATATTCTGCGAAATGCAGGTATTGAAGTGTTTAATGTTCCT
AAGAAAAACTTGAATAAGTTAAACTGGGAAAATATCAACGAATTCTGTGGTGAATAATGA
AATTTCGTTTGGTAAAGCTCACAGCAATTAGTTCTTATTCCAACGAGAACATTTCATTTG
CTGTAGAGTATAAGAAGTATTTTTTCTCTAAATGGAAGCAGTATTATAAGTCAAACTGGG
TTTGTATTGATAGACCACATAGTTGGAAATCTGATTTAGAAAAATGCCAAAAATTACTTT
CCACTCTTAAAGAACGTGGAACAACTCATATTAAAACTGTAATAGGTAAATAAATGAAAC
TGACGACTGAACAAAAAGTAGCAATTCGTGAAATTTTGAAAACTAAATTGTCCATGGGTG
TTTCAAACGTAGTTTTTGAAAAGTCTGATGGTACTATTCGTGTTATGAAAGGTACTCGTG
ATGCAGACTTTATGCCAACCATGCAAACTGGTAAATTGACGGAATCTACTAGGAAAGAAT
CTACTGACATGATTCCAGTATTTGATGTTGAACTTGGTGCATGGCGAGGTTTTTCTATTG
ACAAATTGATTTCCGTTAATGGTATGAAAGTTGAGCATTTGCTCCAATTTATTGGTAAAT
AAATGCTTTAAGAATTATTTGTTATTATTAATTCATCTGTTAACAAAAAGGAAAAACGAT
GTCTGAAGTACAACAGCTACCAATTCGTGCTGTCGGTGAATATGTTATTTTAGTTTCTGA
ACCTGCACAAGCCGGTGATGAAGAAGTTACAGAATCAGGACTTATTATCGGTAAACGTGT
TCAAGGTGAAGTTCCTGAACTGTGTGTAGTTCACTCTGTCGGTCCTGATGTTCCTGAAGG
TTTCTGCGAAGTTGGTGATTTGACTTCTCTTCCAGTTGGTCAAATTCGAAATGTTCCGCA
TCCTTTTGTAGCTCTGGGTCTTAAGCAGCCAAAAGAAATTAAACAAAAATTCGTTACTTG
TCACTATAAAGCTATTCCGTGTCTTTATAAGTGATATAAATAATAATATGAATTGGGTGT
CGGAATAATAAGTTAACCGAACAATTCTATGTGGTAGTCTACAACTGAGAGATCTGTCGA
AAGAAGATGAAATTCAGAAGAACGTGACTACCGAGTTTTAATCTCTAACGAGAATTTTTA
AATGATTAAACAATTACAACACGCTCTTGAACTGCAACGAAACGCATGGAATAATGGTCA
CGAAAACTATGGCGCATCTATTGATGTTGAAGCCGAAGCTCTTGAAATCCTGCGTTATTT
CAAACATCTGAATCCTGCTCAAACTGCATTAGCTGCTGAGCTTCAGGAAAAAGATGAACT
TAAATATGCTAAGCCTTTGGCTTCTGCTGCGCGAAAAGCAGTTCGTCACTTTGTGGTAAC
ATTGAAGTAATTTATTGGAGATTCACTGCCTTAGTGTGAGCTAAATCGAGGAGCCGTCGA
ACTGTCTGATTAATGATTTGCGAATCATTATAGTTTTAAGACCCCGGCAGTTTTACGGTG
TACCTCTTGAATGTGAATGATGACGGGTTTATGGTTATCCTGGTCGTTAAATATCCAAAA
ACCTATGTTCCCCTTGAGGGCTTGCGCAGGCAATGCCAATAAGTCCTGCATTTTCATTTA
AAAGAGAATTTATAATGGCAAAACAAGCTAAAGCAAAGAAAGCAGTTGAAAAGAAAGTTG
GTGATTCTAAACGCGCTGGCTACAAGCGTGGGTCGAACTCTCGTATCAATCAAACTGTTG
AGAAGATCATGCGCCGAGCACGTGCGGTTCTTCGAGATGATGCTTCTCGTTTTGGTAAGC
AGAAAGCATAAGTTAAGGACTCCTTCGGGAGTCCTTTTTTATTTTCCAAAGATTGCATAA
AGTTGTTTACAGTATGGTTCCTTTGTGATAGTATTATCTTACACAAACAAAGGAGAATAA
AATGAAAACGATTAATCTGAACGCTACAGTTAAAACTAAATGCTTCAATGGTAAATATGA
TGAAACTATGTGGTTCTTAATGGCAGTTGAAGGTGATATTATTGAAGTAGAAACAACAGA
AGGTATGGGAACAGATTTCACCTTTACAATTCAAGTTCATAATTTCTTTACTGGTTGGAT
TTATGAATTGAATACAGTAATCGTTGGAAAAATTGAACAAAATGAATTAGGTGAATGGTA
TTATGTTACAGCTCGCCAACGTGCCGAACGCTTAATTGAGAAGATGAAAAAAGTTGGTAA
ACTTGACATGCAGCATTGGAAAGTAGTAAAATAATTGTTTACTTTGGTACAGGACATGAT
ATTAACCTGTACCGCAATTAAACATCTTGGAGAATAAAATGAACTACATTAACTTTGAAC
GTAAATATGTTTCTAATGGTATTGCAGGTTCTATTGATACTATTTGCCTCTGGAAACATC
AAAATGGATCAGTATGCGAAATTGAACAGTATATGACTCCTAATTACGTTTATATGCGAT
TTGAAAACGGTATTACAGTTTCAATCACGATGGAAGGTTCCAACTTTAAAATCGCTCTGG
ATGATGATTTCCGTGAACGCGATTTAGGGACTCATCCTTGCTGGAATGGCGTTAATCGCA
AGCTTTTGGTTAAAACTTGGATTCGTCATATTCTGAGTAACAGAGCTAAACCTGAGCATC
TTGAAGCAATCTTTGATGTAGTTCTTAACGAATTTGATATTTAAAATAAATGAGGGGCTT
TGGCCCCTTACTGAGGAAAATGTTATGTTTATGACTACTTATTTTGATACCCGCAAAAAT
TTCTGTGAAGTTGTTTTCTCAAAGGCACCTAAAGACCTTCCTGCACATTTGCAACCTACT
AGCGAATCGATTAAAAACTACGTTGATGTGGTTTGTCCTTTAGAGTTTCGTACTGTAAAT
GGGCGCGATACTTTAGCTATCACTAAACTCAATCGTGAAATTGACATTGACCCTTCAATT
GCACGTGAAATTAATAGTTCTGATATTAATGGCGGTAATGTTAAATCGCACGGTTTTCAG
ATGAGGTTTTAATGAAATTCTTTTTAGGTCAAACTGTTGAATTAAAGGGAGTTGGTATAC
CTGGATTAATTTCTAAGGTTCTACCTTCGTTTAAATGGAGTGGTATTCAAATAAAAGAGG
CTTATATTGTTTCTTGGGTAGATGGAAATGAAGACCTTCGTATGGGCGATGAATTATCTC
CTATCTACGGATTAAAGGAATTAGTATGAATATAATTAATAAGATTTTTGGAATTCAGTA
CATTAAGGTCACATATAAAGTAACAGATAAAAATCCGTATACTGATGAACACGAAGAACC
GCAAGTTAAGTCTATTATATTAGAAAAAGGCAGTGACTGGCCAGTTGAATTTCGTCTACC
AAACTGTGGTCATTGGGCTGATGTTGAAATTATAAGCATTGAAAATGTCTGAGTTAGAGA
TTAGAAGTAATTTTAGGTGGCCATCATGTGCATTAAGTAATTTCGCCCATTGGCCTTTCG
TTATGGATGGTATTCAATTTGGAGGTCTTGAAGGATTCCTCCAAGGATGCAAAGTGAAAA
ATGTTGAACAACAACGTCGTATATTTGGTTTATCCGGGCTTGCCGCCCAACAAGCTGGAC
GAGCTTATGCTAGAGCTCAGGACCGTGGCACGTTGTTCTGGCTTGGAGTTCCATTTTCAA
GATACTCTCCAGCGTGGAAAGAATTATACACAAATGCATATTTTGAAGCAGCGATCCAAA
ACAGGGGCTTTCGTGATGCATTACAAGCCTCGAAAGGAAAAGTTTTGAAGCACAGCATGG
CTAGTGGTCTAACAAAAGATGATACAATACTAACCGAAGCTGAATTTATTGATGTGTTAA
ATCTATTAAGAGACTCTCTATGAAGCCAACTATTTTGACTGATATTGATGGAGTATGTTT
AAGCTGGCAATCAGGCCTTCCTTATTTTGCCCAGAAATATAATCTTCCGTTAGAACATAT
TTTAAAAATGATCCAAGATGAGAAATTTATTTCTCCAGGTAAACTCTTTAATTGTGACGA
AGAACTTGGCGTTAAGCTAATTGAAAAATACAATCGTTCGGATTTTATTCGATACTTGTC
TCCATATAAAGATGCCCTATGTGTAATTAACAAATTAAAAGAAGATTATAATTTTGTAGC
TGTTACAGCATTGGGTGATTCTATTGACGCTCTGCTGAATCGTCAATTTAATTTGAATGC
TCTTTTTCCTGGTGCCTTCTCAGAAGTACTGATGTGTGGTCATGATTCTTCAAAAGAAGA
GTTGTTCAAAAAGGCAAAAGAAAAATATAACGTAATTTGTTATATTGACGATCTCGGTCA
CCACTGTGATCACGCGAGTGAAATATTAAGTGTTCCTGTTTATTGGATGGCTCGAGGGGA
ACGTGACAGTATTCCAAAAACTGCTCAGCGAGTTTATACATGGAACGATGTAGAGAATAA
GCTTTTTTCACCAAAGGAAAATAAAGAAAGTTTTGATAGTGAAAAAGCTATAAAAGATGT
AATTGAAAAGATGATTAAAAACGATTCTTTTCCTTGGAACACTACCTGGAGAACTCCTGG
ATTTAATCCTTATAATCATCTATATCATCCATATCAGACGCATCCGTTTCAGACATGGAA
CTATATTAAACCCGGCGGTATAGAGTATTTGTATAATAGACCTACTTGTGGTGATAATAT
TTTCCAAGGAGCATTCTAATGTTTGTTGTTCACACTATTTATGAAAATGAAGGTAATACT
ACACGTGATTACGGTCACGTAAATCAATTTTTAGATGCAATCCAGAATTCCGATCTCAAA
AAGACGAACGAATTTTTAAAAAATGCGTAGAGCAAGGTTTCATTTATATCAAGCACTGGA
TGCAAGGAAATAAAGTTAGAACCACATATCATAAGTCTTTGACTGAGCTTAATGATGAAT
TGATTTATAATAGAGCTGTAAACCAAACTCTGAAGGATGAATAATGATTCTTAAAATTCT
GAATGAAATAGCATCTATTGGTTCAACTAAGCAGAAGCAAGCAATTCTTGAAAAGAATAA
AGATAATGAATTGCTTAAACGAGTATATCGTCTGACTTATTCTCGTGGGTTGCAGTATTA
TATCAAGAAATGGCCTAAACCTGGTATTACTACCCAGAGTTTTGGAATGTTGACTCTTAC
CGATATGCTTGACTTCATTGAATTCACATTAGCTACTCGGAAATTGACTGGAAATGCGGC
AATTGAGGAATTAACTGGATATATCACCGATGGTAAAAAAGATGATGTTGAAGTTTTGCG
TCGGGTGATGATGCGAGACCTTGAATGCGGTGCTTCAGTATCTATTGCGAACAAAGTTTG
GCCAGGTTTAATTCCTGAACAACCTCAAATGCTTGCAAGTTCTTATGATGAAAAAGGCAT
TAATAAGAATATCAAATTTCCAGCCTTTGCCCAGTTAAAAGCTGATGGAGCTCGGTGTTT
TGCCGAAGTCAGAGGTGATGAATTAGATGATGTTCGTCTTTTATCACGAGCCGGTAATGA
ATATCTAGGATTAGATCTTCTTAAGGAAGAATTAATCAAAATGACCGCTGAAGCCCGTCA
GATTCATCCAGAAGGCGTGTTAATTGATGGCGAATTGGTATACCATGAGCAAGTTAAAAA
GGAGCCAGAAGGCCTAGATTTTCTTTTTGATGCTTATCCTGAAATTAGTAAAGCCAAAGA
ATTCGCCGAAGTAGCTGAATCACGTACTACTTCTAATGGAATCGCTAATAAATCTTTAAA
GGGAACCATTTCTGAAAAAGAAGCTCAATGCATGAAGTTTCAGGTCTGGGATTATGTTCC
ATTGGTAGAAATATATGGTCTTCCGGCGTTTCGTTTAAAATATGATGTACGTTTTTCTAA
ACTAGAACAAATGACATCAGGATATGATAAAGTAATTTTAATTGAAAACCAGGTAGTAAA
TAACCTAGACGAAGCCAAGGTAATTTATAAAAAGTATATTGACCAAGGTCTTGAAGGTAT
TATTCTCAAAAACGTTGATGGGTTATGGGAAAATGCCCGTTCAAAAAATCTTTATAAATT
TAAAGAAGTAATTGATGTTGATTTAAAAATTGTAGGAATTTATCCTCACCGTAAAGACCC
TACTAAAGCAGGTGGATTTATTCTTGAGTCAGAGTGTGGAAAAATTAAGGTAAATGCTGG
TTCAGGCTTAAAAGATAAAGCCGGTGTAAAATCGCATGAACTTGACCGTACTCGCATTAT
GGAAAACCAAAATTATTATATTGGAAAAATTCTAGAGTGCGAATGCAACGGTTGGTTAAA
ATCTGATGGCCGCACTGATTACGTTAAATTATTTCTTCCGATTGCGATTCGTTTACGCGA
AGATAAAACTAAAGCTAATACATTTGAGGATGTATTTGGTGATTTTCATGAGGTAACTGG
TCTATGAAAGCTTACTTAGAAACAATTGTCATTGCTCAAAAAGAAGGTGGAGATGTTTCT
ACTTCTGTATCACAAGTCATTCTCGAATTTGTAGATGCATATGCTTATAATAAATTCACA
GAAACATTTGATGCCTATGAAAAAGGTCCAAAGTTTGAAATATATCGTACTCTCTTACCG
TTAGATTATTAAAGGCCTTCGGGCCTTTAATTTTATAAATAGAATAAACACTAGAGAGGA
TATGATGGAACTAATTACAGAATTATTTGACGAAGATACTACTCTTCCGATTACAAACTT
AAATCCAAAGAAGAAAATACCGCAAATTTTTTCAGTTCATGTTGATGATGCAATTGAACA
ACCAGGCTTTCGTTTATGTACCTATACATCTGGAGGTGATACTAATCGCGATTTAAAAAT
GGGCGATAAAATGATGCATATTGTTCCTTTTACATTAACTGCTAAAGGTTCAATTGCTAA
ATTAAAAGGTCTTGGTCCAAGCCCAATTAATTATATCAATTCGGTTTTTACTGTTGCAAT
GCAAACAATGCGTCAGTATAAAATTGATGCTTGTATGCTTCGTATTCTTAAGTCTAAAAC
CGCTGGTCAAGCTCGACAAATTCAAGTTATTGCTGATAGACTTATCCGTAGTCGTTCAGG
TGGCAGATACGTCCTTCTTAAGGAACTCTGGGATTATGATAAAAAGTATGCATATATTCT
TATACATCGCAAAAATGTATCACTAGAAGACATTCCAGGAGTTCCGGAAATTAGTACCGA
GCTCTTTACTAAAGTTGAATCGAAGGTCGGTGATGTTTATATCAATAAAGATACTGGAGC
TCAAGTAACTAAAAATGAGGCAATTGCAGCATCTATTGCACAAGAAAATGATAAACGTTC
TGACCAAGCTGTAATCGTTAAAGTTAAAATTTCACGTAGAGCAATTGCACAAAGTCAGTC
GTTGGAATCTTCTAGATTTGAAAGTGAATTATTCCAGAAGTATGAATCTACAGCAGCTAA
TTTTAATAAACCAGCTACTGCACCTTTAATTCCCGAAGCAGAAGAAATGAAAATTGGAAT
TAATTCATTAGCTTCTAAAACAAAGGCAGCAAAAATTATTGCTGAAGGAACGGCAGATGA
ACTTCATTATGACTATAAATTCTTTTCAAAAAGTGAGGTTAATGAAGTTTCTGAAAAGAT
TAAAGATGTAATTCTTAATGCGATTAAAAATGAACCAACTACTTCAATAAAATGTTTAGA
GAAATACGCAGCAGCCGTTAATCAATTCTTTGAAGAATATAAAGATAATTGGCTTGATAA
ACATAATAAAACCCGTAAAGGACAGCCAGATGAAGTCTGGGAAGAAATAACTAAAAATTC
CTGGAACGCAGCAAAAACTAAATTCCTCAAACGAATGATTTATAGCTTTTCTGGAATTGG
TGCAGGTCCAATGATTGATATTACTATTGCCCGTGATGGTTCTAAATATACTCCATCGCA
AAAGCGTGGCATTAGAGAATATTGCGGTTCAGGATATACTGACATCAATAATCTTCTTTT
AGGCCGCTACAATCCAGAACGATATGATGTAATGAGTGAAAAAGAAATTGAATCTGCTAT
AAATAACTTAGATTCAGCTTTTGAAAATGGTGACCGTATACCAGAAGGCATTACAGTTTA
TCGTGCTCAAAGTATGACTGCTCCTATATATGAAGCGTTAGTTAAAAATAAAGTGTTCTA
CTTCAGAAATTTTGTATCTACTTCTTTAACTCCTATCATTTTTGGACGTTTTGGAATCAC
ACATGCTGGTATTGGTCTTTTAGAACCAGAAGCTCGCAATGAATTAACAGTTGATAAAAA
TGAAGAAGGAATAACTATTAATCCAAACGAAATAAGAGCGTATAAAGAAAATCCTGAATA
CGTTAAAGTTCAAATAGGATGGGCAATTGATGGAGCTCATAAAGTTAATGTTGTATATCC
AGGAAGTCTCGGAATAGCAACAGAAGCTGAAGTTATTCTACCACGCGGATTGATGGTCAA
AGTTAATAAAATAACTGATGCTTCTAATAATGATGGAACAACATCTAATAATACAAAACT
CATTCAAGCTGAAGTTATGACTACGGAAGAACTCACCGAATCGGTCATTTATGACGGTGA
CCGTCTAATGGAAACCGGCGAAGTAGTTGCAATGACAGGTGATATTGAAATAGAAGACAG
AGTTGACTTTGCATCATTTGTTTCATCAAATGTTAAACAGAAAGCAGAATCATCTCTCGG
AATTATTGCGTCTTGCATAGATATTACAAACATGCCTTACAAGTTCGTTCAAGGATAAAT
CATGGAACTTATTACAGAATTATTTGACGGCGCTTCGGCGCCGGTTGTTAACTTAAATCC
TAAGCATAAAATACCTCAAATTTTTGCTATTCAAGCCGGTGAAGAAAGCGTGCTTCCTGG
ATTTAGATTTTGTACATACACCTCTGGCGGTGATACAAATAAAAACGTTAAGCCAGGCGA
TAAAATGATGCATATCGTAATGATAGGTGTTAATGAGAAACTATCATTAGTTAAACTTAA
AAACTTGGGTGGAAATCCAATTGGTGTCATTAATGCGGTTTTTGATACTGCTCTTCAGAC
AATGAAACAATACAAAATTGACGCATGCTTATTCCGCGTACTAAAGAGTAAAACAAATGG
TGCAGCTCGTCAAATGCAAGTTATTGCCGACCGTTTAGTACGTACTAAAGGAGCAGGTCG
ATATGTTCTTTTAAAGGAAATCTGGGATTACGATAAAAAGTACGCATATATTATGGTTTA
CCGTAAAAATGCTAATTTAGAAGACATTCCAGGTGTACCTCCTATTTCAACTGAGTTATT
CACAAAAGTTGAATCAAAGGTTGGTGATGTTTATGTAGATGTTAAAACAGGTAATGCTGT
TCCTAAAGCTGTTGCTGTTGCTGCTTCTATTGCTTTAGAAAACGATAAGCGCACGGATCA
AGCTGTTATTCAGAAAACTAAAATTAGTCGTCGGTTAGCAGCACAAGCTCAATATTCTAC
TGTCGATGCTTCACTTCAGGGTGATAGCTTCGCTGCTAAGAAATATCAAGAGTTTGAATC
TAAAGTTCCGGTATATAAAGCAGAAGGACCAATGAACTCTGGCGTTATTCAGGTTGGTTC
AAACTTCAGCAAAGGAGCTATCGGTGGTATGAGAAGTGCTTCCCGTTTTAAATCTAGTGA
TTATGAATTAGAAAGCTTCCGAAATCATATTGCATTGGCTCATGCGCGTTTACGTGATCC
ATCTATCAAGTTACAGAGCGATATAACATATCAAGGTTCTCAAGAATATTTAAAGAATAA
AGAATTTTTTGATTATAAAACTGATAAAATTTTAAGCGATCTTGCTGACATTAATATTTC
TAATAGCTTTGATGTTATTAAGAAAATTGTCAATGATTTAGTTAAAGGTTCTAAAGCCAC
ACCAGATGAAAAGGTAGCTATTGTTCAATTTGTCATGAATGGCGTTTATAAATTGATTAA
TGAGTCTGCTTCTCAAGCGTATGAATATGCAAGTACTGAAGTAACTCCAAAAGGATTAAC
TCAAGCTGAGTCTGATGTAATTGAAGATTATTGTGCAGATTCATATGTTGAAATGAACTC
GTTCCTTTTAGGTAAACCGGATTCTACCCGTGAAGAATATATGGAACGAGCTATTAAACA
CATCGAGACAATGGATTCTGCCTTTGCCAAAGGATCAGTTCTTCCTCCAGGAACTACGCT
TTACCGTGGCCAAGAAGTTACCTTTAAAACTTTACGTCATAATATTGAAAATAAAATGTT
CTATTTCAAGAACTTCGTATCCACATCGCTTAAACCAAATATCTTCGGTGAGTATGGTAA
AAACTATATGGCCCTAGATGATTCCAGTGCAGTATTTTCTGGTGAAGGAGAAGGTTCAGT
TGACGCAGAAGATTTGATGCATATGGGCAGTCACTCTGCTTATGCGAATGAAGATGCTGA
AACTAGCGTAGGTATGGTAATTAAAGGAGCTGAGCGAATCAAAGTTATCGTTCCGGGTCA
TTTATCAGGATTTCCATCAGAAGCTGAAGTTATTTTACCACGTGGAATTTTGCTGAAGAT
TAATAAAGTGAGTACGTACATGATGAAAGAAACTGCTTATAACAAGTATCTAATCGAAGG
CACAATCGTTCCTCCTTCTGAACAATTAGAAGAATCGGTATATGATGGCGACCATCTAAT
GGAAACTGGTGAAGTTCGTCCAATGACTGGATTTAATCAATTCCTTGTAGAAGAATCAAA
AGAAGAGGAAAACGAAGTTTCTCAAATCTTGGCTTCTTTGGTTAATATTAACGGAATGTC
TAAAAAGTTCAAAATGTAGTTTACAAGTCCCTCGTGTTGTGTTATAGTAAACTTACTGAC
ATAACATGAGGAACACAAAATGAAATCTTCTTTGCGCTTTTTAGGTCAAGAACTTGTAGT
TGAAGGCGTTATTCCTGCTGATAACGCCTTTAATGAAGCAGTTTATGATGAATTTATCAA
AATTTTTGGAACAGATAAAAAGTTCGGAATTTTTCCTTCTGAAAATTTTTCAAAGCCAGA
ACAGACTGAAAGCATTTTCCAAGGTGTAGTAACAGGCAAATTTGAGTCAGAAGCTCCGGT
AAAAATTGAAGTTTATATTGAAGACAGTTTAGTTGCTTCAGTTGCTGCTTTTATTTCATT
CCGTAAATAAAAACATGGGGACCGAAAGGTCCCCATTGTTATATTGCTCCTAATATTTTA
CTTTGCGAGTTGACAATTCCCGTCATAGTATTAATGTTTGAAATGCTTCCAGCAGTTCCT
CCTAATCTACTAAGTCGTGAAAGCGAATTAGATAGTCCAGTAACGCCTCCGCTATTTCCG
AGAACGCTTTGAATACCGTTTATAGCAGCAGATTCAAGCCATTCAAGCGCAGCTTGTCTA
TCAACTGCTCCAGCCTGCATGACTCTATACGCAAAAGTAACATCAAATGTAGTTATTTGG
TTATCTCCATCATATGATAACTCAGGAGCGCTCACTGACACTGGAATGCATCCGGTGAAC
ATCACTGCAGTATGAGGCAATCCATTACGAGAATGAAGATTAACTTGAATATCTGCCTCG
ACATCCTGTGGCAAAGCACGCAGTCCAGTTACTGGGTCTTGAACAGCGTTAACCCAGTCT
TGCATCGCACGATAGTTACAAGCTTCTGAATCCATTCTAAATGAAATAACCAAAGGGTCC
AATTCTCTCCCAGTTATACGAATATTAGGAGAGTTATAATTCCAGTCAGTTTCATAGGAT
AATCTATTCTCTGGCATTTTTACAGAGTATATCATCAATCCAGATGAATTATATGCCATA
TTAAAGAAGTCAATTAAATATGTACCAACTGTAAATGAGCCCAATAAACTTTGAACTGTA
CGTTGACTCATGGCACCAATAAGATATTTACTAACCCCTGATTTTCTTATCAGTTTTTGT
GTGCCAGCAGTAATTAGCGTGGTAATTCCCTGATTAATATCGCCTTGAGACAATCCTAAC
CAGTCTGAATTTAGACCCAAGTTATTATAAGAAAAGTTGCTAATTGAACTTATCAACGAA
GAGCTTTTAGTTGATGGAGTTGTCGCAAAAACGCAGCTAAACATATTATTACGTTGGAAA
TCTGCGTTTATTGCTTGATTATTAAATTCCTCTAAAGAATACATTAAAAAGTCCCCGCAT
ATAAAGAAGCACGGTTTAACGTGATAATTTCTCTCATAGTAATCTCAAGGGTAAATGTAC
TAGGGAGGTTTGGAGCAATAGCTAATCCGTTAAAGTTACCATTAGGTGTTTTATCAAATC
TGATACTCTGTATTTGACATGGACCAAATATTTCCGTTTTTCCATCAAACTTAGATGTTG
CACCAAAGTTTTTCACCATCCAAATTGTCGGGTTTGAAACTACTAGAACGTTAGTTAAAC
TCGATGTCATTTTCTCAAATAGTGTTTTATTTTTAGCTGCGTCTTCCGGAGATAAAGGTT
CAATTAACGTAGAACGATACCAATCATCTAAATACCCCTTTATTTCAGCAGCATATTGAG
ATTTACCCGTTTCACCATAAGAAAAATAGTTAAAATATTGATAGATATTAATAATAGCCA
TTAAATCTTCTGTTGAACGTGGAGTCAAATCCCATGTAAACACTTTAGTTCTATTTTCAG
CACCACCATACATACTTCTGGCTGTCGTATAAATCTGTTCATTATTATCAGCCATTATAC
CTTGTGTTATACTTTCCAGCGCCCCAAATACTGCGGTTGAAGCAATATTACTTAGCACAC
CAGTAGCAGTACCTCCGCCTCTACTGATAAGGCTTTCTTGAACATCATTAAATCTATGTG
ATGATGTATCAACATCAGATTTAGATCTCGGTAAAAGAATGTTTGCGATAGGAGCTTTAC
TTATTGTTCCTGAATTATTATTTGATATTAATCCATTTGATAGTTTTGATACTGTACTAC
TAATAGTGTTTCTGGCTGTACGTAAAATACTCGAAGATGAAGAAGAGTAGTTAGATCTCA
TCGATCTAAGACTTCCAGAATCCCTAGATGACATATTGTATGCAGTAAATAATAATCCAT
TCTTATATAGATCTGTCACCTGGAAGTCCCCTGTAGTGTCATTACCACTAGCACGCCCAG
TTGGAAACTGGGCTGTGTATGTTTTAGTTCCTACTTCTGATTTAGTACTCTGCCCGGCTG
AAATTTTCTCACCGGACTTTTTAATTAAATCAGCAGTTATTTCTTTAACAATTGCCATAT
TATTCCTTAATTAACCCCAGTCGCATCAAATACACCAGGAGCAGTTGTGCTTGTGACAGG
TGTCATATTATGAACGACAGTATTTTTCTTAATAACATTATTAGTATTATTGATCGAAGG
AGATGCTTGTTGAACAGGAGCTTGTTGTGCTTTGTTCTTTTCTATTACCTGGACTTGTTT
TGCTTCTGGTGATTTAGCAGAAGTTTCAGGTTTGGCAGTAGGCTGATTTTTCTTGAGCTC
TTGATAAGTAGCATCAATTTTAGAAAATCTAGCAGCAAGTTCTTTTTTAACTGCCGGTGA
ATTATTTAAATCAGGGTCATCCATCCGTTTTTTAAGGTCTTCATAGGCAGATTCAACTGA
TTTAACTGTTGAGTCTTTACTCATATCAGCTGAATTAGCGTATTCATCAAAACGATTCAT
AGCAGCACGAGCTTCATTAGCTTTCATTAAAGCAATTTTAGCTTCCTCCGGAGAAAGTTG
CTTTAATTTTTCTTCTTCCTCTGCCCTCTCTTCATCAGTCGTTAGTGCTTCTTTATTATC
AACGCCACGAATCCAGTTAGATGCACGAGTTTTCCAGTTCGCAATTTTGTCTAACCCTTT
TGCTATCGGACCAAGTTCTCCATTCATTCTTTTATATTGATAGTTTGCAACTTTTTCTTG
GTCTTCTTTGTTAAGAGAAGCATTTGTAGTATTTTGGAAATTTTCTAGTGCTCTTCCTTC
TACTTCATCAGCAGTATCCTTCATGCCAGGAATGACTCGAAGAATTGCAGCAGATAATTT
AGCCATTCCAAGTTGAATAAGTTCACCTAAATTATAAAGAACCTTTCCAAGTCCTTCTAC
AATAGCTACCGTCAAACCGCCCCAATCTCCCGCTTCCCAGAATTTTTTAATTTCATCAAT
AGAACTAAAGATGCTTTGTAATAAAGGACCCCATGTTCCGGTTTCGCTAGAGAATTTAGT
AAAGTCTGTACTAAACAAATCCCAAGCCTGCGAAAATTTATCTGACCAATATTTAAAATG
AACCATCAACAGGTCTATTCCGACAACAACAGCCAATATCATTGCAGTCATTTTAGCAGC
TTCAATAGCAGCGCTGACGGTATACTTAAATAACATGCTTGATATTTTATCAGTAATTGA
AATGGATTTCTTAAATCCAAAATCAACAGTCTTTGTTAATTTATCTAAAGCTTGAGATAA
TTTTAAGTTAAATGCGTCTTTTTTCTGTTTTTCTTCTGGCGATTCTTGTTTGGGCTCAAC
TGGCTGAGGGGTAGGGAAAAAATCAGCATCAGGATCATTATTAACTGCTTCAGGAGCCGG
TAATAAAGGACCCACAGATTCAGCTGTATCATCCTCAACAACTTTAACAGGAATAGCGCT
TTCAACCGTGGCTAAACTAGTTCCTGTTTGTTGAATTCCGGCTGTCTGAATTTTTTGCTC
TAGTAAACTTGTTAATTTATCTAATTTGCTCCCGAGTGATTCACCGATTTCTTTATTAAT
ATTGTTGCCAATTTCAACAGTTTCAGCAATTAACTCGGAACCAGCAGTAGTATCACTCAC
TGCGTTTTCTACGTTGTCAATTGCTCCAATTATTTCATTCGATTTTTCTTCGACAGTTTG
AGCAATTAATTCAGAAGCAGCTTGAGCATCATCCAATTTCGTAGATATATCGTTAAGTCC
AGATAAAGTATTGGAAGCGGATTTAGCCGCTTCCTGTGTTGGTTTATTATCTGAAATAAC
TTTTCTACGCATCGTTTGCATTTCTTGTGGCTTTTTCATTCAAATAATCCAATAATATTG
CCAATTCCAGTTATTGGACCATTAGGGCCAGGAATTGCTAAAGTTGTAAAAATGTCATTT
GCCCATTTTAAAACGAATGCTGGCATCTCAAGAAAATTAATTTCTTTAACTTCATCATTG
ACCTTAAGCAAGCATTTGGATAACATATCGCTCACAGTTAAAAATTGTTCAAATTTTCCA
GGAGGTCTAAAATAAAATGTATTTCCTTGGTATTGAAATTCTAATCTTTGGCACACATAA
ACATCATTAATGTCATAAGTATAACCATCTATTTCTTTACGAGATTTAATCTTTCCATTA
AATTCCAATAAATGAATAGAAACGAAATCAACTTCTGCCGGTGATAAATTCGGACAAATA
GAATCAATAAGAAGTTTTAAATTTTCATCAGGACCTTTAACATCTTTTAAAATGTTATAA
TGTTTAAGACCCATTTTAGGAATAGAAACTTCTTTATTGCTTATTGGAAGAACTACTTTC
TTCAGTGGTAGTATCAGATTTAAATTCATTTTTAACCTTAACTGGGTCTATTGTTTCCAG
TTTCGTTGCATTAGTGAACATATATAGATGAGTTACTGAATTATTATTGGATAATTCATG
GATGACTTCATCAACGTAAAATTCTGTTTTAAATTGGTTTTTACTATCATTAAAAATAAT
TTTAACGCCAGGTGTCAAGTTAAAATTACCGACAGTAGAACATTTAGCATAGCCATCGTA
TTGCGCCATAGTTTGAAGACGAATAGCTTCTTCATATCCATTCCTATAAGTCATTTCAGA
ATAAGCACCTGACCTTGACACTACAATAGAGTTTTCACCTTTTCCTGTAGTAATCATTGG
CAATGAAGAATCTAAAAATGAATGCGCATAGATAGTAGCATTTTTCATTGGGTCACGTTT
GTGAGGATTCGATTTAGTCAACCAAACGAAATCATATGCTAATGGATATTTTAATTCTTG
GATGAATTGACCTATTAAAGATGGCTCACCGACAATCATTGGGTATGGTTCTTGATTTAT
CATCATATCATAGTCCATCATGTTAACTCCCATGATGTCTTGCCATACAAATACAAATTT
GTCACTTCCTACAGCTAGAGCAACTTCTCTGACATATGACAAATAGTTTTCAAATGTACT
AGTCCACGGAATATCAGGAACATAAGCATTTATAGCATTTATTGCTGGAGTCAATAGCGT
ACGGTCTTGATAAATGACGCCAAGCATTTCCTTTATAGATTCACCTGCATCAGGGAAAAA
CGGTCTGCCAAATTTAAGATTTTCTATAGAATGAATAGTTCCCAATTCAATAGCAATGAT
GTTATCACCCTTTGAATCTACTGACACGGAAAAATGCTTACATCCATAAATTCTTGTTTT
AACATTATTAATATCATTTGCATTAGCTACAGAAATCTGAATTATTTCATTTCCATCCAT
TTTTGTATGGATGTTTTTAGAATCATAAAACTGCAGCATTCCTTCATTTCGGCCATAAAG
AGAATCCCGCATAGTTAACGTGGTAATAGTAGCAGCTAATTCAACAAATCTATTATTACT
CCAAGCGTCGTAACTATCAAACAATTTAACGCTGAGATTTGGATATCCTGGGCGTTGCAA
CATACTCATTATTGTTTATCCTTCTCAATCAGTTTTAATACGAATCCGCGCTCGGCAGGA
ATCATTTTCATTATTGAATTTAAGCTATAATTACTTTTTACAAGTGTGTGATTAATTTGA
TAAAAAGTAAATATTTCATCTGGGTTAACTAATAGCTTAAACACATCTACTATATCAGTG
TATTTTTTTACATACTTATTACAACACGACATATGCAATGTTAAATTAATAGGATTCATT
GCATCGAGAATTTTTTCTAATGTTTCTATCTCGATTGCATCAACAAGTTCTATTTGACTT
GATTCGCTAATTTCCTTCCAATCATACCATGTTTCATCTACTTGAACAGAATGAATATTT
TCAGTAATCATCTTTGCTTTATTTTCATAAAACTCAGAAGGAAACTTTAATTTAATTTTA
ACATTAGCTACATCAAAAACAGGTTCCTTTAATTCTTTTTGATATATTTCAAATGGAACT
GTCTTTTCTTTTTTACATTTTGGACATACAAATGTGACCGGTACTTTAGTTTTACCTATT
GACCCTACAAATACCTGCAAAAATATAAATGGTTGCCAAGTCTTCGGATAGTCTCCAAAA
TAATCGGCAATTAAGTCAGCAATTATTTCTTTTTGTTCTTGTGGTGACCGATGTTCTATA
TCGTTTCGAACTAACAAAAAATCTCGATAATCTTCTACCGTAAATGGTTTAAAACGATGA
ACACCATCTGGTAATTTACAACGAATAATGTTTGCCATAGATGCTCCTTTTATTCTATTT
ATAAATATGATAAATAAAGGAGCTAAATATGTATGAATACAAATTTGATGTGAGAGTTGG
TTCTAAAATAATCAACTGTCGCGCATTTACTCTTAAAGAATATCTAGAACTTATTACTGC
TAAAAAGAACGGTTCAGTAGAAGAGATCGTTAAAAAGCTAATTAAAGATTGTACAAATGC
AAAAGATTTAAACCGCCAAGAATCAGAACTCTTGTTGATTCATTTATGGGCACATTCTCT
TGGGGAAGTTAATCACGAAAACTCCTGGAAGTGCACCTGTGGAACTGAAATACCAACCCA
TATAAATCTATTACATACACAAATAGATGCACCAGAAGACCTCTGGTATACACTGGGTGA
CATCAAAATTAAGTTCCATTACCCTAAAATTTTTGATGATAAAAATATAGCCCACATGAT
AGTATCATGCATAGAAACGATTCATGCTAACGGGGAAAGCATTCCAATTGAAGACTTGAA
TGAAAAAGAGTTGGAAGATTTATATTCTATCATCACAGAGTCAGATATTGCAGCTATAAA
AGATATGCTTTTAAAGCCTACTGTTTATTTGGCTGTTCCAATTAAGTGTCCAGAGTGTGG
AAAAACCCATGCTCACGTAATAAGAGGCCTCAAAGAGTTCTTTGAGTTACTATAATGGCA
AATATTAATAAGCTTTATTCTGACATTGACCCGGAAATGAAAATGGATTGGAACAAAGAT
GTTTCCAGATCACTTGGATTAAGGTCAATTAAAAACAGTCTTTTGGGAATTATTACAACA
AGAAAAGGTTCAAGACCGTTTGACCCTGAATTTGGATGTGATTTATCAGACCAGCTTTTT
GAAAATATGACTCCTCTTACTGCTGACACAGTTGAGCGCAATATCGAAAGCGCAGTAAGA
AACTATGAGCCACGTATTGATAAATTATCAGTTAATGTGATACCAGTTTATGACGATTAT
ACTCTGATAGTAGAAATACGCTTTTCGGTCATCGATAACCCTGATGATATTGAGCAGATA
AAACTGCAATTGGCTTCCAGTAATAGAGTATAATGCTTCACGTATAAACGTGGTATAATG
AATCTAAGTCCATCCAATAACAATTGAATAGAGAACAATATGAGATTAGAAGATCTTCAA
GAAGAATTGAAGAAAGATGTGTTTATAGATTCGACTAAATTACAGTATGAAGCAGCTAAT
AATGTGATGTTATATAGTAAATGGCTTAATAAGCATTCAAGTATTAAAAAGGAAATGCTT
AGAATTGACGCACAGAAAAAAGTTGCTCTTAAAGCTAGATTAGACTACTACTCGGGACGA
GGAGATGGTGATGAATTTAGTATGGATCGTTACGAGAAATCAGAAATGAAGACAGTTCTA
TCAGCTGATAAGGATGTTTTAAAGGTTGATACTTCATTACAGTATTGGGGAATTTTATTA
GATTTCTGTAGCGGAGCTCTTGATGCTATCAAATCACGTGGATTTGCTATTAAGCATATT
CAAGACATGCGAGCATTTGAGGCTGGAAAATAATGAGATATAGCATTGATGATGCTTTTA
ATTATGAAGAAGAATTTGAAACGGAAATTCAATTCTTAATGAAAAAGCATAATCTTAAGC
GTCAGGATATTCGTATCCTAGCCGACCACCCGTGTGGTGAAGATGTCCTTTATATTAAAG
GAAAATTTGCCGGATATCTTGATGAATATTTTTATTCTAAAGATATGGGCATTGATATGC
ATATGAGAGATATGGGGTAAATATGACAGATTATAAAGAAGTATTTCGACTATTAGCAGA
GATGGAAGCTTGCTTAATCCGTGTACAAGAAATGCGGATTAAAATGGGTTTATAAATATT
ACTATATTCAAAAGGAGACAATCATGTCAGATAAGATTTGTGTTGTCTGTAAAACTCCAA
TCGATTCTGCATTGGTTGTTGAAACAGACAAAGGTCCTGTACATCCTGGGCCTTGCTATA
ATTACATTAAAGAACTACCAGTTTCAGAAAGTTCGGAAGAACAATTAAATGAAACACAAC
TTTTGCTATAGTGTGACCTTTAGTCTATAGTTTTGGCCCTTCCTTTTTGGTTGGGCCTTT
TTTAATTTAAAAGCTTTCTTCTACTTCATCGTCTGAATCTTCTAATTCAGCTCTTTTTCC
TGCCAAAGCATCTCTCACAGAGATGTCATCAGTATCTTTTAATTCAGTTTCTTTGACTCT
TTTCTTGTAATAAGCTTCAAGTTCTTCTAAACCTTCTAATGTTTGACAAGAGGCAATTTT
ACCCATAAATTCATCAATAGAAGCTTCATAAAGAAATTGTTTAAATTCTAGTAACATCTT
TTTCTCCAAAGGGCCGAAGCCCTTATAAATTAACTGTTTTCATTACGTAATTAAATTTTT
CATCTGCGTAGCGCTGAATACGATCAATGCCGTGTTTTAAAAGATAGTTCAAATGAACGT
ATTTCTTTTTCGTATTAGCAGATTTTGGCTTAACACCGCAATCATCTATGAGGTCCCAGA
CCGTTGCGATTGTTTTAGAACCATGCTTACGTAATACGCGACCTATTGTTTGCAATACAA
TAATTTTAGATTTAACACCGTGCGCTAAAACAACGTGATGCAGATTTTTAACTGAAATAC
CAGTAGAAAATACACCATAACTAGCTACTATAATTATTCCTTTACCGTTTTCAGCTAAGG
TTTTCATTACATTGCGTGTTTCAGTATCAACTTCCCCTGATACGTAATAAACTTTATCGT
ATTCATTTTTAATTAAATCGAAAATAGCTTTACCGTGCGATACGTGTTTAAACATGACAA
AAGCGTTTTCATCTTTTTGTGCAAGCTTAATAGCTAATTTAGCGATCCATTTATTTCTTT
TACTAAGCCCAGTAATAATTTTTATTTCTTCTTGGTAAGTTTTTCCCTTTAATTTAGTAG
TGAACTCATCGGGATAGCGAAGAAAAATACTATTAATTTTTAACTCAGTTACTTGTCCAT
CTTCCATTAATTTAGAGGTCGTTACTGGCTTAAATATTTCACCGAACATTCCAACATACT
GCATGATATTGGCTTTGCCATCACGTAATGAACCAGACAAACCGAATTTGAACATGCAGT
TATTTAAACCTGATATGATAGATGAAATACTTTTTCCTGTAGCAAGATGACATTCATCAT
TCATCATCATTCCAAACTGTGAGAACCATTCTTTCGGTTGTTTTACTACAGTTTGCCATG
TACCAACAACGACCGGTGCATCATTTTTATATTTATCATCTTTTGATGCTCCACCACCGA
TTTTCTTTATCATTGCATGACTGAATAAACGATAGTCAACAAAGTCATCAGCCATCTGAG
TTGTCAGAGCAGTTGTTGGAACAATAATAAGAATTTTGCCTTCATAATTTTCCAAATAAT
ACCGAGCAAGCAAAGCTTGAATTAAAGATTTACCTGCGGATGTTGGAAGATTAAGAATTC
TACGACGATTAACTAATCCTTCGAACACTGCATCTTTTTGATACCAATGCGGTTCAATTC
TTTTATTTCCTGAATAGATTTCTAATTTAGAAAGCCATTCATCAAAATCTTTTCTTGATA
ATTCTTCTTTTTCGTTAATCTGTGGGTCAATCCAGGCTTTATAGCCAAAGTTATCGCAGA
ACTTTTTAATTTGCCCGACTAAGCCGAATGGAAGAAGACGATTATAATCTAAAAGACGGA
TTCGTCCATCCCAGTTGCCATATCTGAAGCGAGGGTTAAATCTATATCCATCGGCCTCAA
ACGAAAAGAAATCTCTTAATTCGTGGAACGTGCTCTCTTCACAATCGATGCGTACATGAC
TGAAGTCGTGAAAATGTACTTTAATATCCATAATTATGCCTTACTAAATTTGCCTTTAGA
ATCTCTTTTCATGAGACGACCTTTAATAAATCCGTCGGGAATAATACCGTCTGGTTGTAT
TAATTTATTTATTGCGCCATTATTGACCCAAAAAGTTCCTGTGGTAGTCGGTTTGACTTT
ACATCCTTTTCTGGACTTTCTATTAGGATGAACCGTCCCTTTTACAAATCCTTCTGGAAC
AAGTTCTCCAGGTTTAATAAAAATATTTTTAGTTCCATTAGTGTAACAAGTTGTACCTAA
TACCGTGCCCGGTGAGTTTTCAAATCTCTTAGCGGAAGATTCTTTCATCTTTGCTATAAC
CTCTGTAGTCATAACAATTCCACCAATTCCACCAGGTTTCATATTATAATAATTTTTGCT
TTTTATTATTTCAGGAGTTATAATTTCTTCTTCATACATGTACGCTTCTTCGGAAGTTTT
AAACTCTTTTAGTATTGTTCTAGAGAAATTGTTTTCACCATATTTCTTTATAGCCTGCTG
AATTGCCTTACCGGAACCAAGGTAACCATCATTCAAGTCATCAGTAGAGTGTTTTCCTAT
ATACTTTTTACCATTTATTAGATTTGTTGTTTCATATACAAAGTGGTACATACTATTTTC
CGAGTAATAAATATATCTATATTTATACTGAGGAAATATTATGATAGATAAAGATTATAT
TGCAGAGCTGAAGGCTCTTGATGATAATAAAGAAGCTAAAGCTAAATTAGCTGAATATGC
TGAACAGTTTGGTATAAAGGTCAAAAAGAATAAATCTTTTGATAATATCGTTAATGATAT
TGAAGAAGCTCTCCAGAAGCTCGCTAGTGAACCTATGCCAGAGACTGATGGGTTATCTAT
TAAAGACTTAATTGATGCTGCTGATGCCGCAGACGGATTAAAATATGACGATGAAGAAGT
CAATCCAGAGGCAGCACTTCTGATTGATTCTCCGGTTAAATCTGACATTAAAATTGAAGT
AGTAGAAACGGATAAAATTCCTGAAAATACCACTGTTTTAATTGAAGATACTCCTTTTGT
TGAAGAAAAATTCGAACAGGCTGTAGCTGAGATTATTGAATCTGAAAAGCCGTCTGTATT
TACTCTTCCGGAAAACTTTAGTCCGAATCTTCAGCTGATTGGAAAAAATCCAGGATTCTG
CACTGTTCCTTGGTGGATTTATCAATGGATTGCTGAAACCCCAGATTGGAAATCTCACCC
AACTAGTTTTGAACATGCGTCGGCACACCAAACTTTATTTAGCTTAATTTACTACATTAA
TCGTGACGGATCAGTTCTAATTCGTGAAACACGTAATTCTTCTTTCGTAACATTAAAATA
AGGATAACTTATGGCTTTTACAGTTGATATAACTCCTAAAACACCTACTGGAGTTATAGA
TCAGACTCAGCAGTTTACTGCTACACCCAGTGGTCAAACTGGAGATGGACCTATTACATA
TGCTTGGACTGTAGACGACGCTCCACAGGAAGAAACGTCAGCAACTTTTAGTTATGTACT
AAAAGGACCTGCCGGTCAAAAGACTATTAAAGTAGTTGCAACCAATCAAGTTGCAGAATC
TGAACCTGAAACAGCTGAAATTAGTACAACTATCACAGTTCAAAATAAGACACAAACAAC
TACTTTGGCAGTAACTCCTGGTAGCCCTGATGCTGGAGTAATTGGAACCCCAGTTCAATT
TACTGCTGCCTTGGTTTCTCAACCTGATGGAGCGTCTGCTACATATAAATGGTATGTAGA
TGATTCTCAAATTGGTGGAGAAACTAACTCTACATTTAACTATACTCCAACTACAAGTGG
AGTAAAAAGAATTAAATGTGTAGCGCAAGTAACTGCAGAAAATTACAATGAAAAGGAAGT
TACTTCTAATGAAGTATCATTGACAGTTAATAAGAAGACAATGAATCCACAGGTTACATT
GACTCCTCCTTCTATTAACGTTCAGCAAGATGCTTCGGCTACATTTACGGCTAATGTTAC
TGGAGCACCTGAAGCAGCACAAATCGAATATTCATGGAAGAAAGATTCTTCTCCTGTAGA
AGGGTCAACTAACGTATACACCGTTGACACCTCATCTATTGGAAGTCAAACTATTGAAGT
TACTGCAACCGTTACAGCAACCGACTATGATAGTAAAACAGTTAAAGCAACAGGCCAAGT
TCAAGTGACTGATAAAGTTGCTCCGGAACCAGAAGGTGAATTACCTTATGTTCATCCTCT
TCCACATCGTAGCTCAGCTTATATCTGGTGCGGTTGGTGGGTTATGGATGAAATCCAAAA
AATGACTGAAGAAGGTAAAGATTGGAAAACTGAAGATCCAGATAGTAAATACTACTTGCA
TCGTTACACTCTTCAGAAGATGATGAAAGACTATCCAGAAGTTGATGTTCAAGAATCGCG
TAATGGATACATCATTCATAAAACTGCTTTAGAAACTGGTATCATCTATACCTATCCATA
ATCTCAGGGGCTTCGGCCCCTTTCTTCATTTTGAAAGCACACAAACCACAATCAGAAAAC
GATGTATATAATGGCACCAACTCGATAACATGAGATTGATTATGAGAACTGAGGTTGTGG
TGTTTACACTCTATGAGTCTGGAAAGTCATTCATTGAAATTGCTCGTGAATTGAACTTAC
AGGCAAAGGAAGTGGCTGTATTATGGGCTCGAGCTATGACTGCTAAGAATAAATTTGAGA
CTCGAGAAAAAGTTGTCTATAGAAAAAGACATATTAATAAGAAGGTGAAAAATGGAACAG
TATGATCTTTATGAAAATGAATCTTTTGCTAATCAATTACGCGAAAAAGCACTTAAAAGC
AAACAGTTTAAGCTAGAGTGTTTTATTAAAGATTTTTCGGAACTTGCTAATAAAGCAGCT
GAACAAGGTAAAACATATTTTAGTTATTATTGCACTGCTCGCGATAAATTGATTACTGAA
GAAATTGGTGATTGGCTGAGAAAAGAAGGATTTAGCTTTAAAGTCAATAGTGATCAGCGT
GATGGTGATTGGTTAGAAATTACATTTTGAGGATTAATTATGTTTAAAAAGTATAGCAGT
CTTGAAAATCATTATAATTCTAAATTCATTGAAAAACTTTACAGTTTAGGATTGACTGGC
GGCGAATGGGTAGCTCGTGAAAAGATTCACGGTACAAACTTTTCATTGATTATTGAGCGT
GATAAAGCGACTTGCGCTAAACGTACTGGACCGATTCTTCCTGCTGAAGATTTCTTTGGG
TATGAAATTATTCTAAAGAATTACGCTGATTCCATTAAAGCAGTACAAGATATTATGGAA
ACTTCAGCGGTTGTATCTTATCAAGTTTTTGGCGAATTCGCTGGACCTGGCATTCAGAAG
AATGTCGATTATGGCGATAAAGATTTTTATGTATTTGACATTATTGTTACTACAGAAAGT
GGTGATGTGACTTATGTCGATGATTATATGATGGAATCATTCTGCAATACATTTAAATTT
AAAATGGCTCCACTTTTAGGTCGCGGTAAATTTGAAGAACTTATTAAATTGCCAAATGAT
TTAGATTCTGTAGTCCAGGATTATAATTTTACAGTAGACCATGCTGGATTAGTTGATGCA
AATAAATGCGTTTGGAATGCCGAAGCAAAAGGCGAAGTATTTACTGCTGAAGGATATGTA
TTGAAACCTTGTTATCCTTCTTGGATGCCTAATGGCAATCGTGTAGCTATTAAATGCAAG
AACTCTAAATTTAGCGAAAAGAAAAAGTCTGATAAGCCTATTAAAGCTAAAGTTGAACTG
TCAGAAGCTGATAACAAATTGGTGGGAATTTTAGCTTGTTACGTTACACTGAATCGTGTG
AATAATGTTATTTCTAAAATTGGTGAAATTGGTCCGAAGGATTTTGGAAAGGTGATGGGA
CTAACTGTTCAAGATATTTTGGAAGAAACTTCTCGTGAAGGTATTACTCTAACTCAAGCG
GATAATCCTTCTTTGATTAAGAAGGAATTAGTTAAAATGGTACAAGATGTACTTCGTCCG
GCTTGGATTGAATTGGTAAGTTAAATAAAAAGGGACCTTTCGGTCCCTTTGTTTTATTCA
TCAATGATAATTTTTGGTAGCTTAACACCTAATAAAACAGACAAATCTGAACGACCCGCC
ATTTTATCCATGTCTCCACCATCAATTACTCTTGCTTCTTTTTCATCTTTTGCTACAGTG
TAAGGATTTGCAGATAAAGCATATCTAACTAATAAACCGATAGATGGCTGCAAACTTTCT
GGATCAACTACAACTTTAAATGCACCTACATGTTCAGGGTCATCTAAGTCAAGACCTTCT
GTATACGGAGCATAGAAAATTGATCCAACAATTTCTTTTTCACCAATATTTTCTACTACG
CCAACGATTACATAATCTAATGGACTGTTGGTATCGCAATAAAGCGGTAAACCATTAGCT
AAGAACCCGTAGGCATTTTGTGAAAGATATTTGTCATCTTCTGGTTTATGTTTTAACCAA
CCTGATGCAGCAAGAATCGCAGCGGCACGAGCTGAAGCAACGCAGAACGTTGCTGTATAA
GTTGATTCTTTTTGGATATGCGAAACCATTTCGCACACCATTCGATATAATGAACGACCA
GCTTCAGGTGCAGATGCATAACTCAAATCAATGAATCCAGTATCAGTAATTCCTGTAACT
TTATAGCGTTTTGACACTGTAATCAAAGACTGTAGAATATCTTTATTGATTTCATCCGCC
ATTTCAGTTGCAAGCAAATCTTCCAAGAAATTAGGAGCATCGAATCCATTTGCTTCTAAA
TCTTGTGCTAATTCAACTGTGATGCCAGTTTTAAGTTTACGAGATTTAACTGCGGTTTGC
CATTTATTAATCTGGAATCTAGCATCAGCAATTTCGCTATCAGAGCTTTCAAATTTGCTC
GTTGACGCTGCATCAGAAAATAGACGAACCTTTAAAAGAACAATTGCAATCTGAAGAGCT
AATTCTAAATCGCTTTCTTCAATGGTAGCAAATGGTGTATCTTCTAATACTTTATAAACG
ATATTATTATATTTGAATAAATCGCCTTTATTGAGAGTTAATTTAGACTCTTCTGTTAAT
TCTGTGATTTGTTCTCGGTCTACATATCCAGCTTCGCCAGCGTAAGTAGCACCAGTTTTA
AACGTAAATTCGTTATCTGGGTTAAGGTATTTAATACCATAAAAAGCAGCAACAGGTTGA
TTAGTTCTTTGCGTTGCTACAATGTCAGAATATATTAATTTAGTGGTAGCGCGAGTCAAA
GCAACGAGATTTGGGCGACCGATTGAGTTGCTATTCGTTGTGGTTGATTCGCGCAGAAGT
TCGTTGATTTTAGCCATTGCGCTTTCCTTTTGGTTTATATGATTTATTTATACCATAAAA
ACAACTAAAGGGACCCGAAGGTCCCTTAAATCGTCAAAGATTAGATACCTTTAACATATA
CACGGCGGAAGTAAGCGTTTTTACCAAGGCTGTTCAGAATAGAAGGCATACCGCTCTGGA
TGCGAGAAGCCGGAGCCTGAGCAGCGGATTCTGCAAATGGGTTGATACCGATACCGTAAC
GAGTTTTGAATCCCATTACTGGTTGGAAGTTCTTCGGATCGGAACCACGCAGCGGAGTCA
GAGCTACATATGGAGCGTAGTAAATACCAGCATCCATTTCGTTCGGGCCTTTATAACCTA
CAGTGAAATAATCCTGTTTAGCATACTGGTCGATATATACACGGTATTTACCACCCAGAA
CACCAGCAAATACTGACTTGGTAGTATCAGTGTTAAAGCCGGTAGCCAGACCCTGTGCAG
CATAAGAAATGCCAGTATCAACTGAAGCCAGAACGTTAACTACGTTACGGGAAGCGATAA
TGAAGTTACCTTCACCACGACCGGTCTGACGAGCAATTTCAACTGCTTCTTTGTCAATCT
GGAACAACAGAGCTTTAAAGGATTCACCCGCCCAGCGAGCACCACGAATATCAATTGGAT
CCTGGAAGTCAAATACACCAGCTTTAGAACCCGGAGTCAGGGTCATACCAGATTTACCAA
CCTGAGCTGAGTAGTTAATCCAATCAACAACTTCACGGTTGATTTCCAACATGATTTCGG
TAGCCAGAATACCAGACAGTTCAGCATCAGCATCCATACCGTGAACAGCGCGGAGGTCTT
GTGCTAATTCGATAGAGTAAGCAGCTTTCAGCTGACGAGATTTAGCTTCGATAACTTGTT
TATCGATACGGAAGCCCATTTCATTCCATGGGTTATCGGTGGAACCGTTGAAACTTTCCT
GAAGTTCAGCGATAGAAGTAGCCATACCTTCAGCGATTTCTACCAGTGCACCAGCTTCCA
TTTGTTTCTTAATTTCAGCATCTAATTTAGCTGCATCATCTGCAGTAGAACTAATTGTTA
CTTGAGCAGAAGCTTGCAGATACACAGTACCAGTGTCCTGGAAGAAGTGAGTATAGATAT
CACCTACTGTGGTTTGTGTGCTAGCAGCCAGAGCTGTGAATTTCTTAGCAGCACCCTGAC
CAGAGAACATTGCATCTGGACCATACATTGGGTGGAATGCTTCTTTAGCGCCGGCAGCGA
TTGGGTCTTTACCATATACTGCGCGCAGTGCGAATACCTGACCAGTTGGGCTGTTCATCG
GCTGAACACCGCAAATATCGAAAGCAATCAGGTTAGGAATAGCACGACGTACCATACCCA
TAACAGCTGGGCCAATCTGAGTTACTGCGCCAGAAGTCTGACCAGCAGCGATGTTAGTAG
CGTTGTAACCGTGGTCACCACCGATTTCAGCTTCTGTTAAGAAAGAACCGAATGCCTGAG
CAATTTTTTCGTCTTTATATTCCGGAGCTGTCTGGAAATCTTTTTCCTGGTTTTCAAAGA
TTTTAGCGATAATCGCTTGTTTGCTATTAGCAATTTCCGGTAAACCTTCACCTTCCAGTA
ATGGCTTCCATTTGTTCAAAAGTTCAGCTTTAGTTTTGATAGTCATTTGTGTTAACCTTT
AAAATTAGAAACGAGATGCGACTTTCGCATATACACTTACAATATCTTCTGCACCAGGTG
CAGCTTTATCTTCTACAGCTTCAGTGACGAAATTCAGTCCGGCTGCTTCAGTATCAGGAG
TATTTATACTCTCAGTAATAGTGCTTTCATCTTTATTAGACTTCTTCACCATTTCTACGA
TGGCACTCAATTTATTTGAGAATTCATCTGAATAATCCATACCTTCGACCAGAGCAGAGA
CTTTTTCTTTTTGAGACTCAGTCAGATCTTTAGTACTTTCGCTCAATGCCACTTCACGCT
GCACATAATTGATATATGCGTCACGCTTATTGAGTTCTTCGAACAGACGAGCTGATTCTT
CTTTATGTTCTTGCAGTTCTTCTTCCATTTCAGCTACAACATCAACTGATTCTTCTGGAA
CAACAACGTTGTGTTCAACGAAGAGCTCTTTCAATCCACCAAGCATGGATTCAAACAGTT
CGGCTTTGATACCTTTATCTACTGCTAATTTATTTTCAGTGAGCCATTCTTTTGCAAGAT
GGTCAAGGAATTTAGAAGCTTGCTCAGCGATTTTCTTCTCAGCTTTTTCTTCTGCTTCTT
CTTTATTTTTTTCTACTTCTTCTTCTGCTTTTTCAGCAATTTTAGCGATATGAGATTCAG
CTAATTTAACGGCGTGCTGCTTGACGGTAGCTTCGAATACAGTGCCGAAAGTTTCTTTTG
CTTCCGGAGAAATATTAACTGATTCGAAAATACTATCAAGAGCAACGGAAGCATCAATTT
TTTGCGCTTCAGCAATCAGTTGTTCTTTAAGCATTTTGTAGTCCTGTTGTTTAGATAATA
ATATTTATAACGCTTTTTTCATGGCCTCTGCGAGAGCCATATAGGCGTCATCGGCACTTG
TATCGGCTTCCGCCGTCTGTGATTCGGTAATTTCCTTAGGAGTTACCCATGCATCTGGAG
CACTTGGACCCCATACTGCATCAACACCTACAGTTAATTTGAATCCTTCGTTTACGATAC
GATAACCTTTATTTGTGTCAGTCAATGAACCTAATCCACGAGAAGAAACTCCTGGAATCC
ATCCGGCACGAATATTAGCTGCTAATTTATCTCCAGGACCGTGGTCACCTTCAATAACAC
GAGCTCGTCCGTATACGTCATTTCCTTTCCACCACATATCTTCTATAATGATAGCGGCTT
GCATCGGGTCAACATTAGCGCGTGGAGGATGATTTAATTCTCCGAGAGCTTGTTTAGTTA
AAACTTGCTCATTAATATAGTCTTTTACCGCTTTTTCTAATATACGTTTTGGATAAAGAC
GTTTATTTCTATTGACGACTTCCGCTTGCATGAATATTCCTTCGATGTATAAACCCGGTT
TTAAACCTAAGTCTTTTCCATCGTGGGATTCGAGCATTGGCACACCATCAATAATTTCGC
CAGGTTGACCCCAAGTTTCAATTAGTAATTGGGGTTCATTCATTAGCTTAATCCTAATGC
TTTACGGCGTTTAAGAGCTTTTTTACGCTTACGCTGAGCACGAGATTGACCTGCTGGATT
GGCAATCTTCGTTTTGGTAGCTTTGCGAGCAATTTGTCTACGTTTTGCTTTAGACAGCCC
AGTGGTTTGAAATGCATTACGTTCACGTGTTTTGCGGTCTTTAGTACGTGTAATTTCACC
ACGAGCTGAAACATGCTTAACAATGAATTCATTTAACGGCATGTTTTCATTAATAGAAGC
TAATGCAACTGCTAAATCAGTTTCATCATCAAGCATATTCTCGACAATTGTATTTATATC
GTCTTTATTTAAAGCAGAAGACAATTCGTCAAAGCGACCCTGTGCTTCAGGAATAAGTGC
TTCGACATTCTCGAGAACTAATTCATGAGTTTCAGGGATCAGAAGCATTATTCATCATCC
TCGTCTTCGTCAGAGTCTTTGTCTTTTTTGTCGTCTTTATCATCACTATCTTCGTCTTCG
TCATCATCCTCGTCATCAGGTTCTTCACCTTCGATTAAGAAATTACGAGCGATAGCGATT
TTTTCTTCTTTAATTAAATCAGTCGTTCTTGCAGCCATGGCTTCAGCAAATAATTTACGA
GCGGCTACGAGGTCGTTTGATTTAATAGCTTCAATTAAACCTTCCATTAAAAATCCTCTT
GTTCTTGGTCGGGGTCTTGGAAACGAGCCTCTTTAGACTCTTCTTCAATTTGCTTGGCTT
CTTGTTCTATTTCTTCATCAGTCATCTGCAAAATATCTTTCATAGCAGTTCTGTGAGAAA
TATATTTACCAATAAATGGTTCTGCCATGGTTAGCATATTAATTCTTCGTTCCAAAATTT
CTGCTTCTTTGAGCTCAGCAAAGTAGCTATCCCGATGAAATTCTATCTTAATATTATTTA
TTTCATCATTCCACTCATCTTCTGTGATTATACCTTTAAGCAAAAGATTTGTTTTAAGTG
GATCTAGGAAAACTTCTTCAAACTTGTGCTGTAACTCACGAATAAATTTAGCAAACGTTA
ATTCATCACGTGTAATGCTAGTTCCAGAATCAAACATCACACCGCCTTGTTGGTCTTGTG
GAATGCGTGAAAGAGGAACACGTAATGCCATGTAAAGAGCTTGTCTAAACCAACGAATAT
CTTCCATATTACCAGTATTATCAGCACCAGGAAGAGTATCAACTTCTGTCACAGCTTTAC
CATCACGGCGCTGCAACCAATAGTCTTCGGTCATAGACATATTATGTTGTTGATTTTTTA
TTTTACCTGTTGATGCATCATATACTACACGGTTTTTCATCGTGTTCATAACATGTTGCA
TGTGCTCAGCAGCTTTACGAGCAGGCATATTACCTGTGTCTACATACCAAACACGACGGT
CAGGAGCACGAGTAATGCGATAAATGACTACAGCATCTTCTAATAATTTTAATTGGTTAG
CAGGTTTAACAGCACGATGTAAATACCCGATGATATTTTTGCCACAGCAATCGACTAATC
CAGAATGGGCATAAACAACAGCAGCTTTTGGAATTTTTATTTTTGTGCCAGCTTCATACA
TTCTACCATCACATGCATATGACTCATGGGCAGTATCATATATAAAATATTCTTTGTAAC
CTTTAACTATTTTTGTGCCAGCTTCAGTTTCTGTTATAATTTCGCGAACATACTGAACTT
GGCGAGGGTCTAATCTACGTAATTCTTTTATGCCTTCTTTTGGACGTTTTGGATCAATGA
TTTTATGAAAGAAAATTCTTGAATCAACATACCAACGTCTAAAATGATCAGAACCTTTTC
GTTGAAATGATAAATGATTTAATACATCATTAAATTCATCTAACATCATATTTTTAATTT
TTGGGCTAAATTTAGATTTATCCAAATTTAACGCTACGACTTCAGTATCATCTTCGTAGA
CGATAGCATCTGAAACGATTTCTGAAACTGCATTATCCACTTCATAGTTATTCATGAGAT
TACGATATGTATCAATAAGCTCACGAGTAGTTTTCATTCCTGGTTCATATGAACCAAAAA
TTGTTTGGAATGCAGCATTATAAGGAGAAGCAGCTTCGTTCGAGCTTACTTCAAATTCTC
TTGCTCCATCATCAAGCTTTGGGGCTGTAATGGAAACAAGATCTTCTTTTTCTTGGTCTT
TAAAATTTCGTTCGTCCATTTTAGCCCATGGAGCAAACAAACTTAATACATTAAATTTCA
TTGTATTCTCCAAATGGGAATTATAGTTATATTTATATGGACTTCTCTGCTTTAAGCAGG
ATGGGGATTTCTCCCCATTCATTTTTATTCCCAATAATCGAGAGCAAGAGTTACTTCAAA
GGTTTGGATTTCATTGTTTGAATCCCAATCTAATTGAAGTTCACCCACGTTAGTAGGCCA
CAGACCTTTAATTTCAATTTCTTTTGTTACTGTTTTAGCATCACGAGCATATTGACGAAC
AATAGCGCTCTTTTTATACTCTGCAGGTTTTCCACCAGTAATTTCGTTTCCTTGCCCAGC
AGCAATGCTTTGCCAATCAACAAACTTCTGGCGTGCATCATGCGCCTCATCATTCATTAC
TGTAATAGTCCAGTCATCGAATGTACGATCGCCTGCTACGTTAATTTTACGGTTCATAAA
TCCGACTGGAATTTTTTCTACAATACCAGCTGGTAAAGCAGTGGCTTTACATTGAAACGT
AAAATTTTGTCCAAGATAAGAAATTTCTACTTGGAATAAGTTAGGTCGAGCAAAATCACC
AGATTCAAACGCTCGTGTTACATCATCTACAAACATATTAGCCTCTGTAGTATTTATATC
CCTATGTTTAACCTAGGGCATATAGAATTAAAGAATTAAGGATATAGTGTATTTATATGG
CCTGCCGAAACAGGCCTTTAGAATGCACCGTATTAACCTGCAAGACCAGTTAACTCATCG
AAATCTGCACCAGTAGCAGTTGCGACGAAATTCAAAGTAATATAATTTATACTACGCGCC
GGTTGGATGTAGAATGTTGCAACAAACTCATTTCTATCAATTACTGACGGAGTGTTATTT
GTTGTATCGCAAACTACACGATATTCATAAATTCCACCGAGAGCTTTAATTCCCTGTAAG
TATTGGGCAGTTTCTGTGCGGAATGATGAACGAGTAAACGCGTTGTTTAATTCGAACAAA
CGATATTTTGAACTACGTCCGATATTCGTTTTCAACATATTAAACAGACGACGAACGTTA
ATACGATCAAATGGAGAAGGAACAGAAGTAGCTGTTTTATCACCATACAATACGTAACCA
TCACCACCTGTACCAGTTACTGGGTTGATAGCTTCTTGATATAAACGGTCGCGCTGAGCT
TGGCGAGTTTCAATAGCAAGTTTAATAACGTTAAGAATCTGACCACGATTATAACCAGCT
GGAGACATCCAAGTCTGAGAAACGTTATCAGTTCTCGCGCATAAACCAGCAATGTCAGCT
GCTAATGGAACCCAACGATTTACATCATTATATTTGTCATACTGATATTTGTAGTTACCA
TCAATTGCTGCGTAGGTTGAACTAATATTAAAGTTATTATCAGTATATGAACCTGCCGCA
GTTCTCCAGTTAACTAAATTATCTACTGCACGAGTTACAGGAATTCCAACAACAGTTTCA
CGCGGTGGTGAGCACAATACTAAGCAATCTTGGCGAGCATCTCCAATTGAAACGACGTGT
TTTTGGACAGTAGATGCTGTTTCTAAAGATTCACCGGCACAAGAACCCGCAATAAACAGT
TGAACGTCAACAGATTCACGGTCAGCAAAGAAGTCCCAAGCTTCCATCAAATCTCCTGCT
GTAACTTCAGCATTTGATGATAATCCACCAGAAAGAGTTAAAATTCCAGAGAATCCTTCT
GGCCAGTTTTGTGCAGTTGCAAAAATATATTCTGAACCACCTTTTGCGAAAAAGTCATCG
ATATAGATGTTACTATCGTAAATATCTTTTTCACCACGCTTAGTTGAAAGAACAACGCTT
TGAACAATAGCATCATTTCGACGAACTATAATAGCGTACTGTGAGTCAGTCTGCGGCCCA
TATCCAAATACTGCCTTGGCAGTAGATGCGCGTGTTCCGCCACCTGGATAAATTGGGAGT
AATGCAGAAGCACCTTTTGCGTAGTCAGCTTTAGATACGATTTCAATTTCGATTTTATCG
CCCAACTCGCCTGGATAAAGAGCTACTACTCCTGGAATTCCATATTTTTCGAGATTTGCC
TGAAAGTCAACCGCTGTCATAGCAGCTTCAGCATTTTCAATCTCAGCTAATAAAATACCG
GAATCAGTAATAATTTTTCCAAGAGTTATTACCGCAGCTAAACCAGATGAAGACGAAGAA
ATTTCCGCGGTCCAGTTAGAACCTAGTTCTGGATATTCACCGACTTCTTTCGCTTTAGCG
ATAATTTTTGCAGTAGGAATATTAATTTTCTTAATTTTTCCATCAGCATCTACTTCGGTA
ATTTTACCTTCTGTTTCAATGTCTTCTGAAACATACTTAACTGTGATTTTATCTCCAACC
GCGTAGTTACTACCTGGGGTAGAAATAGTGTATTCAATATTACCAGCAATCGGAGATGAG
TTTTTAGCGGTATCTCTATCGACAGCGCGCACAACTCGCAAATCATTTCCATATTGTAAG
AAATTCATTGCAGACATAAAATAATCAGCAGTTTCAGCTGTAGGTTGACCAAAAGTATTA
ACTAAATCTACTTCATTTGTAACCTGTTTAATCTGAAAAGCAGGACCCCACTGGAATTTA
CCGGCCAAAGCTGCTGTACCAGTAGAGTTATTAACCACGGTGCTTTGAACCGTAGTTTCT
TTGAGCTCAATGCCCGGAGATAATAAAGTCATTTTTAATCCTCTTTAATATGCTTTAATA
TATTTATACCATTGACATACCATGAGATACTGGAACATACTCAGCAGAATGAACCGAATC
AACAAATATAACTGGAGCGTATTCATCACCCATATCTTGAAGTTCTTTTGAAAACACTTC
AGATGCTAATCGCATGTCATCTTTGTCAGCATAATCAATAAATTTTGATTGTGTTGATAA
CCATCCAAAAATCACTAAAGACATGACTAAATCGTCATGATAACCTTCTTCAGCTGCCCA
AGACACGCCTTTTTCACTAAACGTTCTAAACTCTTGAATAGTTGCACGGTGATGAATAAT
AAGCTTATCTTTTTCAATAAGGTCTTTTAATGTAGAGCATCCTACTGCTTTCGTTCGTTT
AGTTTGCTTCATTCCTAAATCAGTATATGAATCGCAAATAACGCCTTCGTATTCTAAATC
CATATAAAGTGATTTCGCAACTGACACACCAGTACTATTTAATTCAATATAAACTGGGCA
TTCATTATATTCTACTAAATATCGCATAACGATGTCAGGTAGAATTAAATGAGAAATAGT
GTTTGAGTGTAAAACACCAACCTGCTCCCACACATCATCGGTGACATCAATAATATGTAA
AGCATGATAATCTTGCCCACGACCTTCTGAGCAGTCTAGAGTTGCAATATATTTTCTATC
TGGTTCAGGTTTTTTAAACTGATGAAAACCATGATCATCTGGTGTTACTTCTATGAAATC
CATAACAGCTAATTTCATTCCTGAAATTAATGTACCAGAAGTCCCTTCAAACGCTGCGGT
ATGTTCTTGACGAAATTGAGCTAAAGTAGAACCATTAATGGTTTGTATGCTCCATTGCCA
TCCATCGTCAAAAATATCTTCATCATTATAAAGACGTTCTTTAACTGAATTCCAAATAGC
AGTGTATGGTTCAAACCCTGATTTACCTTCAACAGCAGCAGTCCAAATATCATAAAAATG
ATTTAATCCATTAGGAGTCGTAGTAATAATAATTTTCGAACGACGACCAGATGAAATTAC
TGGTTGAATAGCAAGCCAGGAATCATGGAAGTTTGGAATAAATGCGCATTCGTCAATATA
AATCATTGCGAACGAGTTACCACGAACTGCGTCAGGAGAGGAAGCGTAGGCTCCGATTGA
GGAACCATTATCAAGTTCAATAGACCCTTTATTCCATTCAACAATTCCTGGCTGTAAAAA
GTCAGGAAGCAGTTCAATTGCTTGCTTAGTACGATCTAAAACTTCTGCCGACATTGAGCC
TTTATGCGCCAGAATACCTACAGCTTTATCCTTGTTAAAACATACAAAATGTGCAAGGAA
AATAGCTACTACGGTTGTTTTACCGAGCTGGCGTGATAGATTACAAACAGTCATACGTTT
AGATGACATTATTTTGAGCATATCACGCTGATAGTCACGTAATTGAACCTTTATAACACC
ATAGTCAATATGTGTAATAGCACAGTATGTTTCTGCAAAATAAACAATATCGTCTCGGCA
TTTTTTCCACTCCTCTACCATTTCACGAGTCCATTGTGTTTTAATATTAGCTCGTTTCAA
GTTAGGAAGACCCATATACCGAGATCTTTTATTATTCTTATCTTTATAAGTTTGAAATAA
TTCAGGCTTGTCAGAATTATTTGGAATTTTTACTATCTTGTGTAGACGAAGGTAATCACT
GAATTTTTCAGGATACCATTTTCCATCCCACTGAGATTTTATCCAATGAATTCCATCTTC
ATCTTTTCTTTCAGCTAAGCTTGGGTGTTTTATTAAAATTTTTCCAGCTTCATTTAATGG
ATGGAAATCATTTAATGCATTAATCGGTTGTTCCATTTATCACCTTCTCACGAGCTTCTT
GAGCCTCGTAAGCATCACCAATTTCGTCCATTAATTCTGTTGGTGAACCCATGAATACTG
TCGCATTTTGAATATTCATTTGACCTGTAGGAACAGCACCTTTAGTGCCAACCTGCTCAG
ATGTAATATCTTTCATATCTTTATGAAGCTTCAGTATTTCTCTGTTCGTCGTAGTCATTT
GCCCCATAAGAGTTGCAAATACTTCCATGTGACGAGGAGAATCAGCATTCTTTGCCGTCT
CAAGAAAAATCTTGGCTGCGTCCATTAGCATTTGCTGTTGAAAATGCATATTTCGACGAA
CTACTCCATAATCATCTTCTAAGTCAGGAGTACGGTTTTGAGGATTGCTTTTAACTTCTA
CTAATTGCAGAGGTTCATATACTTTAATTTCCTCCCCGTCAATTCCGGGGAGGTCAGAAA
TATCTAAAAGTTTGTTTATATCAAGACCTTCCATAATAACCTCTATGTTCTTGGGCCAGG
AGGTTCTGGCGGTGTTGGTCTATTTACGTTGCTAGTGAAAGTTTGTTTTACCGTTCCATC
CCAGTCTTCTGGGTTAATATCTCGAGGAACAACTTCGCTATCGACAGATTCAAAAACACC
TTCACCATCAGGCAAATCTCTTGTGTTGGCGTGAAAATCTGTATAAGTAGTACGAATTAA
TCCTTCTGCATCATCTACCGGAGGATACATCCATCCATTTACTTCAAATGTTAGTGACCA
TTCAATTCTACGACGAGATAAATTATCTCCATCTATAGATTCGTCTATAGCAGCAGACAT
CAGTACAATTTTAATATCCCTTTTAAATGGAATATCATTTCCAAACTGTTCGTACATAGT
TGTATTAAAATGAGGCTGAAAATATGGAAGAATTTGTTCAACTATTTGAAACATATCATC
CTCATAGCGAGTAAATATACTCAATTCATAAATCATTTTAATAGGAGATGGATTATATTG
TGATACTACAGAAGTTGCGCCTTTTTGTAGTAAATTTTGATTTAAAATGTTTGTTTTAAA
TGGAGCATTATAGCTAAAATCAACTAAATGTAAATTTATACGAGGTAGAATAGTTTCAAC
TTTAGCCACATCTTCTTGTGAATTTATCGATGTCCATTTATTTAATTTCATCATGAAGTG
TTCCTTTGATGCATACGTAATAGGAACACGTATAAACTTATCACCAGATTCTAACTGACG
TTTGATTTGGATATTTGAAAACAAATCGCCCATCAAGGTAGCATATCGTCTAAAAGACGA
ATTATAAAAATAACCAAACATGATTTCTCCTAATCTGGGCTTTAATTAGTTTATAATATT
TATTAATCCATGAAATCATTATCAAATGGGCTAGATTCAAAAGATTTGCCTCTGTTATTG
ACAACAACATAAGGTTCAACATATTCTTTAGCTTCAGAATTAATTTGATCTACTTCAGCA
TACTGATCAATATTAATATCATGAATGCCATTAAGATTGCGAACAGGATTTAATTCTAAT
TCACTGAATTCCGGAATGTTAATTCCTTCATTTTTCTGTAGAACTGGATTAATTTCTTCT
CCAGAATAAATGAATTTACCTGCTGTAATTTTACGAATAGCGTTTTGGCCTAATTGATAA
AATGGATCATATGGTTCAACCCAGTTAATTTCAAATAAGCTGTTATCCATAGGAAAATAT
ATCAAATCACCTTCCTTTGGTTCTTTTCCGTTGACTTGATGTTTAAACAAATTTGGATTA
ATGGACAAAGTTACTTCATCCTGTACTTGCATACCAAAATTACTAAAGAATGATTTAGCT
CCTTCATATCCTTCAAATGAATTTAAATATGCAGCGAATTTCCAAGCTTTAGTAAATTTA
TTTTTTAAGTCTTCGCCGAATATCAAATCAGGGGAAACATACTCTCTTGGAACATAATAG
CATTCTACACCTCGCATTTGAATGCTTTCAGCTACTAATACATCGGCCAATATTTGACTA
TTTTTATAATGATTGAAATTTACATAAGGATTTAGTATTTCAGTTTCATTAGTCTGAGAA
TAACCAGTACGATTTTCTAATTTAGCAAAAAGATTTTTATCATAAGTAGCCATATTAACC
TACCAAAATTCCAAATGGAGGATCAAGCAAGTACAATTCTTCGCGTAATGCTTCTTTTTC
TAATCGAGCTTCTTCTATTAAGCGCTGTCCATCAATTGTAACACCTCCAGGAAGCATCAT
ACCTTGGTGACGTGCTAAAATTTGGCCATTCAATTCTTTAGCTAAAGCTGTAGCATAGTC
TTTCACCCAACGGTTATTATAAGCACCTTGCTTAACATTTGGATCTTGACCAACAACACG
ACCTACTAAATTATGGTCTGGATTATTATATCGTTCAGATAATGACCAGCTATCTTGTGG
GCCGACTGTTCCATATCCTACTGTATTTCCAACCATTTTATTAGTATCAATATATGATTT
AGTCCAGCTTTCTACGATAATTAAATCATATTTTTGGAAGTTTCCCATGACTTTGAGTTG
TTCATTTGCTGAATTAAACCAAAAGTCTGGAATAGGCGAAAGCATATCTTGCATCATTCC
CATGTAACTGGTTAACTGAGTAAAATACCCTAAATCAGCCCCAAAGGCATTTGGTCCATA
AAATCTATTGCAAGAAGTCCCCATGCCGCCATTAATACCAGCCATTCCTAAAAGAAAGTC
AGTAAACCACGGATATGTAGCGTTTCCGTCCATTGACGTTATTGACCCAACATTCGTGCG
GAGAATGCGAGTTACTGCGAATACATTTGAACCTCTTAAATCGAAGACTCCGGTTTTATA
CTTTTCTTCGTCATCTCCTACATAAAATACATGAAAACCTTTGTTAAGTCCATCAAAATG
GTATTCACCGTATAATTCTAGGGCACGCTGGATGCAATCGTAGATTTGATCGGGTGTTAA
CTCAACATTGATAATTGGAGCCCCTAAGCGTCTTAGAATGACATCTTTGAGTTCCTTTGG
ATTCTGAGAATTATATCCTGACATTTAAAATCCTTTGGGGCCTTGCGGCCCCATGTTATG
CTGGTGATAAAAAGGTAGAAAGTAGTACCCATTCGCCGTCTTTACGAACGTAAGCTTGGC
CATCTTTTGGAGCTTCAGGAATATAACCTGCTTCTTGTAGAGCTTGAACGCTGCTCTGTA
AAGAGGATATATTGTCTTTAGCTGTATTTACTTCATGTGTGACTGCCGCAATGTTAGTTT
CATTCGTTTTTACAGAATTAGTTAATCCTCGTTCTTCAACAGTTGCTCCATCAGGATTAT
TTCCATTTATAAGTGAAGTAAGTTCAATAACTTGACCTTTAATTCCTGTTCTATTATTTC
CTATTTCAACTTGTATATTTTGAACATTATTGTTTAAAACTGAAACTTCTCCTTCAAGCA
CTGAAACTTTATTTAATAGAGATCCAGAAGGAGACGGTTGTCCTCCACTGGAGTCAGTTC
CAACAATTTGATTTAACCACGAAACATTCGCTCTTAAACCAGACGAAGTGTTTTCACCAA
CAATTCCGTTTAAAGACTCAATAGAAGTTGTGTGATTTTTAATTTTTCCTTTAATACTAG
AAGGAATATCATCTGAACCGATAGAAGTTTCAATAGCCGTTAAGCGCTGTTTAACACCTT
CACTCTGATTTAATTCATTGTTAATAGCTTCAATGCTACTTTTGTTATTATTAATTTCTG
TTATTATAGAAACATTTTCTGGGTATCCAATAGAAGATTTAATTTCTGCTATATCAGAAT
TTATTAAAGTTTGTTTATCATCTATAGTGTTTAATCTATTATAAATATTTGGTCCAAAAT
CTACCGGTTTATTACCAAGTTCATCACGTAATTTACCAACTTCAACAGTCAATGATCCTA
CGTCAGATTCAGTAAATTTAGTTTCTAATTCGCTTAAACGAATGCCTTGTGAAGATAATA
ACGTACTATTTGTTATTATTCTGTGTTTCATTCCTGTGCTAGCATTACCAACAACAGGAA
GACCGTTAATGTCTTGGCCAGAATATTGTCCTAATTCCTGTTTAATCCACAGTAAATCAT
TTCTAATTGTTCTATAAACAGAGTTTTCTTCATCATTAAATGGACCTATATCAGCAATAA
TTTTGTCAACTGTATTATTAGTTCCAGTTAATATATCAGTGTGTTCACTAGTAAGTGTCT
TTAAATTTGATATGTCATTTTTATTAACACTGATCTGCGATAGCGCTTCTATATCTCCTG
ATACGTCTAAAACTCCTTGAATTGTTTTAATATCTTTCTGTGATGTTTCAATGGCAGTTT
TAAGAATTCCCACATTTTCATCAAGAACTTCGACGTTTTTAAACACGGAAACAATTGGTC
TATTCATTGATCCATCATTTCCATACTTAGTACTAGCTCCTAATATTTCTTCACCATTTT
TAATCCATGAAATACGTTCCTGACCTTCGTCAGGAACGCTATCAACAAAAGGTAACTCTT
TTAATTCAATCATTTGTAATCCTTAGTAATGAACTTTAATAATATAGTTTAATGATATAT
TCCATGGTCTAGTTTCATAACCAATTAATCCTTCACGATTAAGTGTACCATGAGCATCTC
TCGGTCCACCTAATTCAAATCCATCATTAGTGAAGTATGAAGCATTATCCCAGTCGGAAT
ATTTTCTAGTTCCAAGATATCCTTGATAAACTGACGCGCCAAATGGGCCTTCACTTCTGT
TATATTCTCCCCAACCACCGGCGTGTTTATGGTATGACATTTGTTGTGCCTGGACACCAC
CAACATGCATTCCGTCACATCCTACGCCAAGTCTATCCTTTCCATAACCATCTTGTCCTC
GTTGATTTAAAATATGACCGCCTGTGCCGGCTCCTCTGACGAAAAGACCTCGCATATCAG
GAATACCAGGATTATTCCAATCACCACCAAATCTTGTTCCAACTACGTTTCTATAATCAG
GAAATTGGTCACCTGACACGGTTCCACCGTGACACATAGACCAACCTGGAGGTGCTGAAT
CACCTGCAAACATCATAATAGTCCCAACTGGAATTCTTTCGGACGCATATCTTTCAGTGG
CAACAGGTTGATCGCCATTTTTCCACATTCCACCACGCGACCAAATTCCGTTTGCTGTAA
GATGGTCTAAATTTAAAGAACCATTAATAGTTTGTCCACCACGAGTATTAATTACATCTG
CGTTCCATGCTAATGCTCCAGAACTATCACCTTCAGGGGCAATACCGGCTTGCGTCGTTA
ATTTAACTACTCCACGCATAGAACCGGTAGCTCCTCGACCATTTAGTGTTTCACCTGTTA
CTGCAACACCCCCCAAATTATTATTAATTTCAGTCTGTGTTCCTAAACGTATAACACCCC
TATATTCATCTGTTGCAACAGAATTCATAAAGGTATACGGGGAAATTGCATATCCTTCGC
GGAGAGTTCCTTGACGAGTTTGCGCAACAGTAGATAATTGAACTACTCCTCTCACCGATT
CTGTTGCAGTGTCTTCTGACGGAGCTATTTGTGAAATTAATTTTATTGCAAGTTTTTGGG
TTTTTAATGGAGTCATTGCCGTAGTATCATCTACGCCGGCCAAAGCTGCAGGCGTAGATG
ATATTTTAATAACCCCGTTTGATGACTCAGACGAATATCTATTTTGGAACACATCGTCAG
TATGGTATTTCAGTTTTTGCGGCGTTATTGATGAATTATTATCTGAGCCTTCTAACGTTT
CTTCATTTGTTGAATAACGCGTTAAACCATATTTAGTTTCAGTAGCATTTGGGTATAATA
ACCTTGTTGCTAATGTAGCTGGCGTAACGATTTTAGAATTATTAGTTCCATCTAAAACCT
CTTGTTCTGACGCAATCATTGCTATTCCTTTAACTTCCATAGTTGCATCAGGAATACCAT
TTACGCCGATATTACTTATTTTTGATAATGCAGACTGCACTGTCGTAACAGTGTTAGGAA
AATTCGATCCAGTAGGATCGAATTTAACATATTTTGATTCATTTGATACGTGCTGATATG
TATTGTTACTCATGCTACTCTCTCAAAATAATAAAATACTGTAGGTTGTGTGTGACTATC
TAGCGTAACAGTTTGCGCACCTAATAATTGCCAAGTTCCGTACCCAGGTTTAGAAGGAGA
AATTATTTCCTGTTTGAAAGTAATTCCTTTATCAGAGTATTGTTCTAAGATATGTTCTTG
GTTATCTAAATACTTAACTTGTATTTTATTTCCTATGGTAGGATGGTCTTCAAATGAATC
AATTGCAATAAATTTTACAACCATTTCCTGTAGAGCTACTCTAACGGCACTAGTAAATTC
GATTGAAGTCATTCCATTAGTAGCTTTAACTGGAATACCAAACATGTTAACAATAACTGG
ATCACCTGGTTTGGCTGAAGTATCAGTAACGGTTCCTTCAAATGACCAATAGTCTGTTTG
TTGCACGCCAGTAGGAGATACTCCGCTTATATTAACTAATACAGCTCCAACCTGTTGCTC
TGCCATATTTCTAACATCATTGATAGCAGATTCTACATTTGGGTCATAAAAACCTTTTGC
TATTTGTGAAATTGTTACAGCGCCTACTGGCTGATTATTCATAACCGAAATATCATTTTT
CTTAGTTCTAAAACCAAGAAAATCTGCTAAGCGGGAAATAACTCCCGCTTTATTATTAAG
TAAACTCATTATGCAATCCTTATCCAACGGTATACAGTAATAGACGGTTGAATATTACTA
ATATTAGCAGGAGGAGTATGTGAAGAGTTTGTTGTTGCGTAATCTTCACGGTATTTTGTA
TATATAGGACCAGTTTCATCTGGGTCATATTGGCAACCCCCAATAATAACCGATCCATTT
TCGTCTTCAATTAAAACTCTTTCGTCGGTTTTAGTTGCAGGAAGATTTGCGTTTTCAAGC
GTTACTGATGTTGTTCCAACTGTTCCACCGGCAGTATGTGAAGGATTTCCGCTAGAATCT
AAATCATTATTGTTTAAAGCAAAGTTTGGATCCGTAACATCATCATTCCATCCTACTAAA
ACTTGTCCTTTACCAAATAATTTCCATGAACCAAATCCCATATAAGTCACCGGATTATTT
GGGTTTATAGCATTTTCATAAATTGAACCAACAGGATAAATGCTGTCGAAAATAGATGAT
ATTGAATTATACGTTCTAGTATAAGAATCAACTTTTTCAACATTTGGCCAACCGATTTTA
TCAAAATCTGTTAAGGCCACATCACCAATAACTTCAGTAGATGATCCTTTGCTAACATAG
CGGTCATCAGTCAATTCAATAATGTCATCTTTTTCTAAAAGAGTTCCTAAATCGTTATTG
AACCATGTTATAACGATAATGTCACCGGATTCAAATTTTCTATCAAATAAAAGACTGGTA
GGTTTTCCATCTTCATATTCTATAGAATAATCTGTGTTTGATTCTTTCCATTCGCCACCA
AGAGATATACATCCTTCTTGTGTATCTGAACTAGCGCCTTCACACAAAAACAGAGGATAT
CCAGCTGTTCCAGCCTGCTGCTGTAAAATTCCATTAAAACGAACCTCAAGAGAGTTAGGA
TTAATGGGTTCACCAGGAATTAATCCAAATGCAGAAAATGGAATTGATTTCATCGCCGAT
AAATCAGTAACATAAATGCTTCCTTCTAAAGAAGTCTTTGATGTTAGTTTTGAATCTAGT
ACTTTTATTTGGCGTCTAGTATATGAACTTCTCCATTGTGATACACCATCCATAAATGTT
TCAATTTGAACAGTGTCACCAATATTACAAGGCTGTCTTAATCGAATATTAAATCCATCA
AGAGGAACTAATTCGCCTTCATTTTCGCCCGGAGAGCCAAAGTCACTGTTTTCACTAAAT
ACATCACCGTAATACAATTCGTTACCACGATGTTTTACTCTGATGTTATTGACATTATAA
CTAGTTCCATTGAAAACATCTAAAAAGTCAGTTTGTCCTTGAACTTCAACTAAAAATTCT
TTGCGAGCTACATTACTAATATCTGAACTAACAATTTTGTCAATTTGTTTGTTTTTGACA
TATTCCCAACGTCCTGGAGCGCAATAAACTAACTCTAAATCACTAAATTGAACATTAATT
TCAACCGATGATGACGACCCTTTAATTGTATCTCCGGAAGCAGCTACTAAAGTAACTGGA
TTGATGTTCCATGTAGCGAATACGTCTCTAGCTCTAATTACCTTGTTGTAATCATTAACA
GTTCCTTTAGGAAGTTGTAGAGTTACTCTTCCAGACGAAGTATTAATGGCATATGATTTT
CCCCATTCTGCCGCTAATGTTTGTCCAGATGAAGCATTATAAGTTTTCCAGGCACCGGCT
GAATATGGAACATCTCCATCACCAAGTTCATAATAAAGTTCATCAAAGTTTTCATTTATT
TTTATACCACCTTTACGCAGGTAGTCACCGGTACCATCATCAACAACATTACCGATATTA
ATATTTTGTTTCATTATTGAGCCACCCCGATTTTCTGCGTAGCGATAACTTTAACTGCTG
CTCTCATACCGACAGTTGAAGAACTTATAGTCGCTGTTACATAATTTGTTTTAATACTAA
ATGCAATATTAGCGATTTCGTCTTCTTCGGTTTCATTCCCAACCCGCATGACAGCATATT
CAGAAGAAATGACCTCTGAATTAACAGTATCTACAAGAATATTTATTTCTGCTGTTTTAA
TTTTTCTTCCATCTACTGATTGGCATGTAACTAACAATTTAGCCATGTTATATTCAGTAC
GATGAAATAGCGGAATGTCAACTGATCCAGACGTAGAAATATTCCATGTACCTTCGGCCG
GTGATTCCTTTTGGCCAAACATACTTTCAATAGAATAATTCCAAACTGACGTAGAATTAT
CAGATGAAATACAACGTAAAGTAACTTTACTATATGGGCTAGTTACTACCAAATTACCTG
AAACACCTTTAATTGAATCAATCGCTTGAATCGTTAAAGGATTGGTAACTGATATAGATC
CATTTGAGTTAATAAATTCAACGCAATCACCAAGTTCGCCTCTTTCAATGATAACTTTAA
CACCTACAGCAGAGGTATCAATATCATGTCTAGTACCAACTTTCACCGGAGTTGCGTACT
CTGTGATAGAATGCTTTTGATAATATCCAGTAGCATGGATAATTTGGCCGTTAGCACCAG
TGCCATTTGCTACTGCCATTTTACGCTGATCGCCAAACGCGTTATAAATTGCGTTAAAAT
CACTATTAATTTTATTACCACCGTCGAATAAGATATCACCAGTAGAAGCGTTACCAATTT
CGCCGGTATCAATCAATTTCTTTGGTTCTTGAATGAACATAGCGGTTTCCTTATGAGTTT
ATAGTATTTATAAAGAAAAAGGGAGCCTATGGGCTCCCTTAATTTAAAATGTAAACAGAA
TATTGATTTCTTCTGTTTGATCCATTGCCATAATAATAGGTGGCCTATTTTCCATATAAA
TCATTTCGCCCGAATGCCTCATTAAATCTTCCGGATCATAGTAATCCTTTTCAGCTTTAA
CGTTTGGGTCATTTGGATGAACTTTAGCTTCAAGAGGATTCGTGATTATCGATATTTGTC
TAAATCCTTTATTTCCTGGCAATGCAGCTTCAGGAAAATAAACTGAATCTAAGTATGCTT
TAAAACGAATAGTATTTGCCTTAACCCGGTAAATTAATCCAAAATCATCTTGCTGCCAAG
TGAGGTTATCTTCATACCCCCATCTAGTCGGATCTTCTTTTAATTCCTCAGGCCAAGGAA
CCACGATATATTCATTCGTGCATCTGTTTATAGATACATCAGGCGGAATCTCAAAAAGAT
ATTCCCATACATACCCGTCTCCAGGTTCGATTGTTCCTTCCGCGTCTCCTCGACCTTCAG
GAGGAGTCATTGACCTAACAGAAGGAGTCCATTTTCCACCTAGCTTAAGGCATTCATCCT
TATTAGTTAAAGACTCAATTGAGCACATTCCGGTATCAGGAACATCTAAACAACGATATA
CTAACCAGCCTGCGCCTGATTCGGTAGCGTTGTAAGGAGCTGAGTTACACACTACAATAT
CGTTAATTCTAAATGTGTATGGATCCGGATATCTAGTATCTCCCCAATCTCGACGAGGAA
TAACTGCATCAAGCATAGATGGAAGAACCTTCACGGTTCCCATCATATGAGTCCACATGT
CAGTTACACCTAACACAGAATCAGTGGGATAAGGTGGGGCAAATCCCACCTCATTTTCGT
TTGATGACCACGGTTCTGATCTTCCAAATGTAATAAAGATAGTGTTTTTATCCGGACCAC
TTCCAATTGAATTATAAAAATTCAACATTTTTTCTGTTCTAAATTTTGAAGTAACTATCG
CACGATAGATAACACTTGAATCATTCATCTATTTTAACCTGTGTTGGATTTTCAGGGTCT
CTTGGATTTCCTATATTATCTTTTAGACGTTTATTAACTAAATCCCTAAACTGTGCAAAT
GTTGTTCCAGATGCGTCAAATAGAGGACTCATTAACTTGCGTCGTTCAGAAGGCAATTGA
CCTTGAAATATTGAATTATTATTTTCAGCATTATAGTCATTAGGAAGAGGATATTTTACA
CCAGCCATTGGGCCAGGTTCATAAATTGCTTCTCCTGTTACTGAATCATGTTCAATTTCA
CCAGTTGGAGTTAATTTAGCTACTCTATCAGCATATTCAGTAGGCAATCCAGAATCCCAC
TTGTAGTTTTTGTATTTATTAATTATAGTCTCTGTATGTTTAAGAGTTAAACCGACATTA
ATAAACATTGTTAAAAGAGTAATTGCTATAAATCCAAATCCTACTGGATGAACAAAGCGA
ATAACGTCAGATTTCCAGCGAGAAGAAGGTAAATTGGATTTAATTTTCATTACATAATAT
GATCGGCTTCTATTAATATAGTCTATATTATTTTGAAGCAAATCCTTTCCTTTAACACCA
CGAATAATTTCGCCTTCAAAACTAGGGAGTCTTTCTGCTTTAACTTCTTGACCGGCAATT
AATCTTCCCAAAAGATTATGAATAGTTACAGTCCATTGCAATTTACCGTTAGAATAACTT
CTTTCTATATAAGTAACATTACATCTTCCTGTAGCTGTATAAATCGTTTGTCCTACTAAG
TCTTCAGTCAAAGAATCAGATTGAATGATTATATCATATTCAGTACCAGCCCCAGATTCA
ATTTCAATTTCAACTTCTTCATTATAAAGAACTTTAAAAAGAAATTTGTATGATGCTTCA
ATTCCTTTAGTAGAATAAAAATCATAGCTACGTGATTCAAAAAATCTTGCAACAGCATCA
CGTTTGTCAGCATTTAAATAAATGTTTCTTTTATATATCTCTGACCACAAATATTCCCAT
GCATGCTCTTCTCGTGGATATTTGTTACGAATTAAATTTCGTAAATTGTTATACTGAGTT
CCATATCCATCAGAAAGATATTGAATATATGCTTCGCAAAATGCCTCAAAATTCGAATCT
TGTAGTAAATAAGAATCAGGCATCATTGTGCCAATCAATGGTCTTAAATCAGGGTCAGCT
AATCCATGCTCTTCTTCTGGTGACCAAGGAGTTTCACGCTCTTGGTTTTGTAAAAATGCT
TTTAAAAATATTCCAGTTGGCTTCCAAATTATTTCAACCGTGTCTCTCACTCGATAGTTA
AAATCATAGTAAGAAATTATTTCACCAGAAGATTTATAAAAAAGCATCCCAGACGCATAT
TTTTTAAATCCCGTGAATTTAACATTTGGCATTACTATCGTACAATCACCATCATCCCAG
TATTCATGAACTAATCTATCTGGCGATGTTTCCGGAATATTTTCTATAACTTTAGTGTAT
TTTAAATCAGCATAAACTACTACAGCTCTGTCAGAGTTGTTTATCCAACAACGTGTGTTA
GATTTTTTAGACCAGTTAAAGAATGGTTCAGCATAATATTTCATTGGTTGCGGAGCAAAG
GTTTCCCAATCAGACTTTTCATCTGCTATAAATGCCATCATATGATAATGCTTATCAGCT
AACCATTCACGAGGAAATTCATATTTAACAGCACCGATTAACTGATATTTCGCTGCAGTT
TCAGGGTCATTAACAATGTTATCACTTAAAAATTTAAAATTACTCGAAGACAGAGAAACT
AATTTACCATCAGTTGACATATTCGCATATCCAGGTTGAATACGTCTTCTTTCTTCTTCA
GTGTTGCCAAAAACTCTTTTCCACGTTTTTGTATCATGATTTAAAACGTATATTCCTTTA
TCATCAGAATCAATTATTTTTGATGTTCTAGGATTAGCATTTAATGTTTCAACTTCACCA
ATAATAAGAGCAAAAACTTTATCACCGATAGAATCCATTTTATAGCATACTGCTTTAGGA
TTTCCGGTTATAGTCATTGTATCAGGTTCAAAAAGTCTTTCCGAATATGTTGGAGATAAT
GGATCAGAATCTATAGGTGCATTACTCGTTTTTATGTATCTAACTTTGTCTCTGGCGACA
ACGTAAATGTAATCATCGGTGCAAGTAATAGCTTCAGCTATACGATACACATTTGCAGGT
AATGTAGCATAAGTACCAAAGATTTCAACATCAAATCCCAAATGAAGTTGATCACCAAGT
TTAGCGAATGTTATATCCTGCGAACTAAATCTGACATCATCAGCTGACCATCTAACATCA
GTAGATTTGCGGCCATAGAAAATCTTGTCGTATCCTAGAACGTATGTTGTGCTCGCAGAT
TGGTAATATACAGTCTTAGATAAAGGATATCCTACACGGTCATTGAAGAGCTTTACAGCT
TTCCAAGTTTGTCCTTTATCATTAGATACTTTAACTACAGGTTGATAACGCTCAAAAAGA
TATAGAATCCCTTCTGATTCCATCAAATAAACTCGATTAATATCCTTACATACTTGCTGA
ATAGAACCTTGTATTTCATGATACTCATTTTCACCAATAATAAAATTACTTATTGATGAA
ACATCAGCATATGATGGGCTGAATTGGAATGATTCATTCATCAATGCAGCCATTATAGTA
TCATTATTAAAATTAACATAGTCAGAATTATTAAGAGTAAATTTTTCCTGAATGAATTTA
TTAGCTAATTGCATTTCAATCATGTTTTGAAATGTATAAGCGTTTGTAGCAAAAGTTTCA
AACTCTTCGGTATAAATCCAATCAGACTGCTCAAAATCCTGTGCAGCTGTAGCTACTCTA
ATAATGTACGATGTCAATGGATTAGCATTATCAAAAAAGAAGCTGTTATTTGCAGTATAT
CCTAAGTTAATCCATCGGTATTGATTGCTCGGGAGATTTTCCCCCGAGTTTGTTTTTGTC
TCAGCGATTTCTACAAAATAGTAGAAATTAGCACCAACGTCATCCCAGCGTACTTGCACC
TGATTTGCGGATAACTTGGAAATTCTGAGACTAGTGACTGAAGGTGCTTTTACTGTCATT
GTGATATAGGCTCCAAATCGATAGTTAAGTATTGTGGACGTAAATCATTTTCAAATACAA
TCAGTGAACCATCGCGTGTGAAAATAACATCGTCTATTGGATCAGAATATAGTTCAATAG
TCTGAACCTCAAATTTATCGGATGTTAAATTAATTTTAGCGATATTCCAATAAATCATGT
CAGCTGGATAATTTATTTCACCGATAACATAGTATTTGTCACGTCCATCGGAACTTGCTA
ATTTATTAAAATCATTGCCTGTATACGGCTGAATGTTTTCATTTTCTGTAACATCGCCAG
AAGCAAATGGACCAATAATAACTTTGCCAATTCCTTTAGAATCTCGGTCTGTTGATACTA
TACGAACGTCATATAATACATCTTCTTCTAAACCAGTATCAGGATTTACAACCTTTCGTC
CAGAATTAAATGAAAACGTATTAGATTTCATAGAACGATCTTTTATTTGATTATTGTATT
TAATACCTGCTTCAGGCGTTTTATAGAAGTTTTGTACTTCACGGACAATTTGAATTGTCG
CGGATGAGCCAATGATAGAATGATCTGCATCATCTACGTATGTCAGCATCTTAGATTTAG
CAAAAGATGAGTTAAAAATTTCTACATCTTCGGTATAATAACGATCAATTTTATCAATTA
TTTGACCTTCGAGCCACTGTTCTGATTCTTGCAGCTTATTTAAAGCATATGTGACTTTTA
AATTAGTCTTAATAAAAAGGTAATTAGGAGAAATAATTGATGGTGTAATAGGAGCTAAAT
TATAGTCTTTGAGATAATTTTTAATATCTTCGCGTTGTACAGTAGTTAAATATAGTCCTG
ATTTAGGTTTAGCAGCAATAAATGCATAACCAGGCTTAGTAGAATCAGTGAAAGTCTGAA
CTGCTTGAATAATAGAACCAAATCTCTCTGAAACGAATGTATCATAGTCAGTCGCAGTTA
CGCATCGTTGCTGAGTTTCGCGTTTAATAGTACCCAATTCGCGAATACGCTCAATATCTT
CTGGATCGCCGCCGCCATCTGCCCCAACAAAATCTGGGTCATCATTTGGATTTTCGTTAA
TATTGATGACAGTTATATTTGTTAATGTATCTGCGTATGAAAATCCGACTGCACCATTCG
CATCTGCACCGTTAGTACTGATGTACTCAATAACAATCGTAGAGTTCTGGGTAGGTTTAA
GACCTCCAATATAATTAGCGGTCAATGCTCCTTCTGCAGCATTAACAGAAATCTCACCTT
CACCAAAATAAAATTCAGTATTTCCATCAATAGTTTCACGCATGTAGTAAATTGTTGATG
TAGAACCAGCATGAACCATTGACTTTCTTGTCCAGTTAATCCATTCTGCTCCATCAACAT
ACAGCTTAACTTGGTTTCTATCTATATTTTTGTCATAAATGATAATAGGTGTTAATTTAT
CATAAATGATTTCAGTTCTTACTATACGTCCCTGGGCTAATTTTAAACGCGGAAAATATT
GGTTATTTTTATCGCGAATAGCAATAACATCTTCGGTAGATACAAAGTTATAAGGATTAA
CAGAAGTATCTTTTGCATATGCTAAAAAGCGAGTACCGCGAGGAATAGTAATGTAATTTC
TATTCAATGCGTCTGTACAAGTTAGCATAATTTCAGTTTGAGCAGCTGATTTTGAAGTAG
GTAAATATCCATTATCTTGTGCAGCTTGAACTACTGAACTACGCAAATTAGCAGTACGCA
TAAAGCTTTCATACAAGGCAGCATTACCAAACTGCTGAATGTACAATGTATTATAAGCCA
AAAGGTCACACAGAACGTTTAATCTTGAGCCTTCAAAATCATAATCCAAAAATTCATTTT
GACCATTAAGCCATTCAATGAGGTTTTGTTTTATTTCAGCAAATGTACCCCCGACGAATA
TCTCGGGAATAGCATTTGCTGTTCTTGTTAATTGATAATTTACAGGGGTATTTGCCATTT
TAAATCCTATTTAATGAATACTTTAGATGATGCCTGAGCCACAGTATCACCGCATGATAT
TGGATCAGCCATTTGAACAGCTTTCTTTCCAGTGACATATACCTTAGAAGTTCTAGGTTG
TGTCACTCCGCCATGTGTTTCATATGGCTTTTTAATTTCTGTATGTTCTGTAATTGGATC
ACCTGCTACGAGAACAGCAATTCCTCCAGTGAATACTTTACTTTGTGTAGCATTCACAAC
TGTTGGAGGCCACGCTTCATGGCCGGCAGTAACACACTTATCATAACTTAATCCTGACAT
TTATGGCCTCTCATATACATAGCTTCTCAATTTATTAGCCCAACGACTCCAATTTCCAAC
TATAGTTTTTGTGTAATTTTTAACAAGAGTTTTTCTTATTGGAGCCGGAGGATTCGTTGG
TTCAGTAGTATCAGAAGATGACCTAAAATTACTGCCAGAACCTCCAGATTCACTTTGTTC
TTGGTAGTCATATATTAATGTTACTTCGTAGGTGAATGTCTTCTGGAGGTTTTGAGGAGC
TTTCCATAGATATAACTGAGTATTAGAATCAGTTGGAAGGTCTTCCCATGAAGCAGCAGT
TTTAAATTCATCATCTAAACGATATTTCAATGCGTCATTTCCAAATCCAAATACAGATTC
ATACGTTCCATATAAACGATTTTCTTCTACTAAAACCCCAGGAGTTTCTTCGTAACTAGT
TATATTTATAGATACTAACGTTTCACCTGTTTCTAATTGAGCGGTAAAGGTGACGTCGAT
AGAAGAACCTTCCATGGATTCTCCTAAATCAGCGCTCATTGGAAGTATATTAGCCAATGT
CAATCCTCGATCCATCAATTGTGTATTGACCAGATGAAATAGAACTCATAGATGCCATTT
TTTCTGTCCAATCACCACCAACGTCCCAATCAACTGTCCCAGCAACTTTCCAAGAAAGAT
TTCCATTTACTGTATTAGTCTGATTTCCTTCAACTAAAGTGGTAGCATCTCCTTTAACTG
TAATGTCAGCATTACCTTCGACTACAATAGTAACATTACCTTTAACTAGGATAGTTCCAT
TACCTTCAACTGTCTTAGTTTCATCGCCACGGACAAATATTGTGTTGCTTCCATCTATTT
GGTGAAGACGATTATCCATGTTGTAGTAAATTTCTGATCCACCGACGTTAGTCTTTTTAT
CACCGGCTACCAAAAAATTACCATCAGCATTTGTTATATCATACAAATTATCAACAGTTT
TTCTTGTTCTTCTTCCTGACGGTGATACTTCCTCATACGTTCCAGTCGGATGAACTAATC
TGTATCGTTCTTGCCCAGGGGTATCATCAAATTCCTGAATATGACCGCTTTCAGTTTCCA
TTGTGTGAACATACGGATATTCACCTTTATATGAAGAAACAGGTTCTTTGAATAAAATTC
TCGAATCATTCGGAATAGGAGGGTCAGCTGGATCAGAAGACTTTGCTACAGTAGCAGCCA
TTGCTGACAAAGACCTAGCTGGTGTTTTAACTTCAATACCGTATGATTCCAAATTCCCTG
TAAGAATAATCATGGTAACACGGGATGCACGGCCTTTTGTTTGTTGATACCACAATGAAT
CACGCCCGGCTTTATATGCTTTTTCCCAATCTCCGGCTAACATAGCAGTTAACATTGTGT
TAAATTTAGCTACGCCGCCAACACCCATCTGAAATGCCATATTTTCTAACGCCATTTGAC
GAGAACGATTAACTGCTTTCCAAACTGGGCCAACTTTAGAATGTGATTTAATGTCACGTT
GCATATCAGCCAAATCGCGTTCAAATAAAGTTACCGCTTCTTCCATTGTAATAGAACCTG
GATTTCCGGTAATTTCACGACCAACTTGTTTTGATAAAACTTTATTAATTTGGGCCATAT
CACGAACTGGCTGCTTCATGATAAGATGACCAATACCAATTGTCGGATATCCTTCGGTAT
CCCAATAAACTTTTAGTCTTAATCCTTCATCGCGGCGAAGCATTTCAGCCATTGACATAT
TTGGATTATCATCAGTTGGAATCTCTGATAGCGGTCTATCATCGGGATTTATCGCAGTGT
CTAAATTACTGTCTTGGATAACGTTAGAAGACGAATCATATCCTACTTCTCCGCCTTGGT
TTAATACGTTAGTATCATTTCCTAGACGTCTAGGATACTGTCCAGTTGGGTCAGAAAATC
CTTCAAGTCTATTAGGTTTTTCGCGAACTATCCCGCCATACGTGCCAAGGACAATTCCGT
TAGTTTTCCATTTGTCTAAAAAATGACCATATACTCTAGTTCCTTCTACCGGACCAGTAA
CGGAGCCTCCAATTCCAGACATTGCTGCAGAAGTTATAGGTTGAATAACTGACATCCATG
GTAATTTATCAGTTGGAATACCCATTACATCACCTTGTGCTCTTTGAGGTGGATGCAGAC
CAACCACACGAACACGAACACGACCTAATTTTAATGGGTCCATTCTATCTTCAACAACAC
CAACAAACCAATTAAGGTTATTACTTATCATTTCCATAAGATTTCTCCATTATACGTATA
AGGTCGTTCATAAATGAATTAATGTCTGATTTTGCTATTATTTTTATTTGACGAAGTTTT
TCATTTTCAAGAACAGCATCTTCATACGTATCGACCGCAGCAAGCGCTCCTTCATATTGA
GGATATTTTCTAGCTTTATCGCCTTTGTCATACCAAACATATGGATTATCATCGTATGAT
ACTAAATTATAAAATTTTTCACCGTTCTCATTCACATGATATACTATTTGGTCTCCACCT
ACATTTTTGTATTTTTGTATAGATGCTTGATAAGCAGCTTCCTGCGAAGTAATCCATCCA
TAATACGGGTCATAATTATCATTACACATCAATAAAACCCAATACAACTGTGGATTTCCA
TATATAGTATTTGCTAATTCTTCCGGGCGTGGTGAACCTTTAATATAATAAGTACGTAAG
CGGTATCCCGCAAGAGCGCGTTTAAAATAGTCTTTATAGTTTCTAAAAATATCTGTCATA
GGAATAGTCGGCGCGTTTTTATTCACCGTTTTGGCCGCATATTCAATCGGATCAAAAAAT
GTAAAGAGCATGGGCCCTCCTGTTTATAAATATATTATCTATTTATAAGGAGAATCCAAT
GACATATTCTGGAAAATGGGTTCCTAAAAATATATCAAAATACAGAGGTGACCCTAAAAA
AATTACGTATAGATCAAATTGGGAAAAATTCTTTTTTGAATGGTTAGATAAAAATCCAGA
AATTATTGCATGGGGTAGTGAAACAGCAGTAATTCCTTATTTTTGCAATGCAGAAGGGAA
AAAACGTAGATACTTCATGGATATTTGGATGAAAGATTCTTCTGGACAAGAATTTTTTAT
TGAAATAAAGCCTAAAAAAGAAACACAACCACCAGTTAAACCAGCACATCTAACAACTGC
AGCGAAGAAAAGATTTATGAATGAAATTTATACATATTCAGTTAATACTGACAAATGGAA
AGCAGCTCAAGCTTTAGCTGAAAAGCGTGGAATAAAATTTAGAATTTTAACAGAAGATGG
ATTACGAGCTCTTGGCTTTAAGGGGGCATAATGGCTATTTTTCAAATAATTAATGAAAGC
ACTCCCCAAGTTCCAAAGGTTAAGCAATCATTAAACGAAAAGAAATGGATTCAGATAGGT
CTTGAATACAAAAAGGCCAAAGCAAAAGGAATGACAGGAAAGCAATTTGCTGAAGAAAGA
GGAATCAAATACTCTACGTTTACTTCGGCAATGTCAAAATATGCTTCAGGAATTAAAACA
GCCGAAAAGATTCAAAAGCTTGAATCAAAACCAATGAATAAACTCAATAAGCAAGAAAGA
CAACTGCTTATGATAAATTCATTCAGACAAACATTGCGTGATAAAATTCGTAATGAAGGC
GCAGCAATTAATAATAAAACCAGAAAGTGGTTTGCTGAAACTATTAAGCAGGTAAAAGGA
CATAAAGTTGTTCGCCCGCAGCCGGGACGAATATATGCTTTTGCTTATGATGCTAAACAC
AAGGAAACTCTTCCTTATTGGGATAAATTTCCTTTGATAATTTACCTCGGTTTAGGTAAA
CACAATTTAATGTATGGATTAAACTTGCACTATATTCCACCTAAAGCTCGTCAACAGTTT
CTAGAAGAGCTTTTAAAGCAATATTCAAATACACCTACTATTACTAATAAAACGAAATTA
AAAATTGATTGGAGTCAAGTGAAAGGATTTAGGGGTGCAGACCAAATGATTAAGGCATAT
ATACCTGGTAATATTATGGGTAGCCTTGTTGAAATCGCCCCGAAAGACTGGGCGAACGTT
GTGTTGATGCCACTCCAAAGCTTCTACTCCAAAGGAAAACGTTTCTCTGCAAACAAAGTT
TGGAGCAATGTACGATGAATTATGAAAAAATTTATAATTCACTAATTGATAGGGCTCGCA
ATAGAGCCCTTACTGGATATAAAGAAAGACATCATATTATTCCGAAATGTTTAGGCGGTA
GTAATGATGCTTCTAACTTGGTTGATTTAACTCCAGAAGAGCATTATGTTGCACATCAGC
TATTAGTAAAAATACACCCAAATTCTTTAAAATTATCTTATGCAGCCATAAAAATGACGG
TTTCAACTAAATATGTTAGGAGAAATAATAAAGTTTATGGTTGGTTAAAAAGAAAACATG
CAAATAATATTTCAAATACACAAACTGGACGAAAATATTATAATAATGGTAAAAGATGTA
TAAAATTGCATGATGGAGATATAATTCCTGAGGGATTTATTTTAGGAAGAGGATGGAGTC
CGACACAAGGAACAACTCATTCTAGAAAAAATGACACATTTAAAAACAAAAAGCTTCAAT
CTGCTTTAGCTAATAAACGGTGGGAAAAAGAAAAGAAAATAATGTTAGATAACTTTGGAG
TTAACACTATTGATGAAGCAAAGAAAATTGTTCTTGATTTTAAATCAACTTGTCATCCAA
GATATTGGCGAAAGCCAACTTTAGAAAAATTTCCATTTATGACTTCTGCTCGTTTAAGAA
GTCTAATAAATTAATTCTATTATCTTCCATTCTTTTCTATTGTTTGTTCTAATTGGAATT
GAATGGAAGGGACTTAGACCCATTATACCACCAACATTTATAAAGCATTATGAGGAATAT
ATGTCGCAAGCACTACAACAAATTTTTAACCAAGCAAATACAACTAACTTTGTAGTATCA
ATACCACATAGTAATACTACATCTGCTTTTACTTTAAATGCTCAATCGGTTCCTATTCCA
GGAATTAGAATACCTGTTACTGATACCGTGACTGGGCCGTTTGGACTGGGCCGAGCACAA
CGTCCAGGTGTTACATTTGAGTACGACCCACTCATTGTGAGATTTATAGTTGATGAAGAG
CTTAAGTCGTGGATAGGAATGTATGAATGGATGCTAGGAACTAGCAACTATCTTACAGGT
GAAAATACTGCCCAAAAAACAGGTCCTGAATACATTACGCTTTACATTTTAGATAATAGT
AAAACTGAAATCGTGATGTCAATAAATTTTTATAAGCCTTGGGTTTCTGACCTGTCTGAA
GTAGAATTTAGCTACACTGAAGATTCAGACCCAGCTTTAGTATGTACAGCAACGATTCCT
TATACGTATTTTCAAGTAGAAAAAGATGGCAAAATTATAGCAGAAGTTTAATGCTTCAGT
TTCATGTGTTATAATCTTAACTAAATTTGAGGAGAAACATATGAAACTAATCTTTTTAAG
TGGTGTAAAGCGTAGTGGAAAAGATACTACTGCAGATTTTATCATGAATAATTATTCTGC
GGTTAAATATCAACTTGCTGGTCCTATTAAAGATGCATTGGCTTATGCATGGGGTGTATT
TGCAGAAAACACTGACTATCCTTGCTTAACTCGTAAAGAGTTTGAAGGAATTGACTATGA
TCGTGAGACTAATTTAAATCTGACTAAATCAGAAGTAATCACGATTATGGAACAAGCATT
TTGCTATCTTAACGGTAAAAGCCCAATTAAGGGGGTGTGTGTGTTTGAGGACGAAGGACA
AGAATCAGTTGATTCCGTAGCATGTAACAAGATTATTGACGTTATAAATAATATTGAAGA
TCAATGGTCAGTCCGTCGTCTGATGCAAGCCCTAGGTACGGATTTGATTGTTAATAACTT
CGACCGCATGTATTGGGTAAAATTATTTGCTTTAGATTATCTTGATAAATTTAACTCAGG
TTATGATTATTATATCGTTCCTGATACTCGTCAAGATCATGAAATGGATGCGGCTAGGGC
GATGGGTGCTACAGTAATTCATGTAGTTCGTCCTGGTCAAAAATCCAATGATACGCATAT
TACAGAAGCTGGATTGCCAATTCGTGATGGCGATTTAGTAATTACAAACGATGGTTCTCT
TGAAGAACTTTTTTCTAAAATTAAAAATACACTAAAGGTACTATAATGTCTGAACAAACT
ATTGAACAAAAACTGTCCGCTGAAATCGTAGCTCTGAAGTCTCGTATCCTTGATACTCAG
GACCAAGCGGCTCGTCTGATGGAAGAATCCAGAATTATGCAAGGAACTTTGGCTGAAATT
GCTCATGCAGTAGGTATCACTAGCGACACTATCAAAGTTGAAGAAATCGTCGAAGCTGTC
AAGAATCTTATTGCTGAATCTGCTGATGAAGAATAATGGAATTTAAAGACTTTTCAACGG
GTCTTTATGTAGCAGCTAAGTTTTCAGAATTAACACTTGATGCGCTGGAAGAACTCCAGC
GCTCTTTACGTGTTCCTAATCCAGTTCCTAGAGAAAAAATTCATTCGACCATATGTTATT
CAAGAGTAAATGTTCCATATGTTCCATCGAGTGGAAGTTTTGAAGTAGCTTCTTCTGGAC
ATTTAGAAGTATGGAAAACACAAGATGGATCGACTCTTGTACTTGTGCTAGATTCTGAAT
ATCTGCGCTGTCGACACATGTATGCGCGGGCACTAGGTGCTACACATGATTTTGATGATT
ACACACCGCATATAACATTGTCTTATAATGTTGGACCCTTATCATTTAGCGGTGATGTAC
AAATTCCGGTCGTATTAGATCGTGAATACAAAGAGCCTCTTAAACTCGATTGGGCAGATG
ATTTAAAATAATTTCACAAAGTTGTTTACACGGGGTTTTAGTTGTGATACTATTACCCTA
TCAAAACAAATGAGTGATTGGAGAATAAAATGAAAACATTTAAAGAATTTATCAATGAAG
CTACTTTTAACGAAAAAGTAAAAATTAGCAAAGCTGTCAAGACTTTGATTGCTGACATCA
ATTACCTATATGATATTGATTTAACTGACTATGTAACAGTTGGTAACAAACGTAACTTTT
TCGTGTTTGACATCTCTGAACTTGACCAGAATCAAATTAACAACATTGAGCGTTTTGCTA
ATGATAAGGGTCTTCGTCATGAGTCAGCGGGTTATAAAAAATACGCAATTTATTTAAAAT
GAGTTTACTTTTAAATAGGGTTGTGGTACTATTATCTCATCAAAATTAATTAGGAAAATA
AAATGAAAACATTTAAAGAATTTATCAATGAAGCGGCTGCACCAAAGACATTCGTTATTA
ATACTCAGGCGAGTCTTGACGATGAATACGCGGAGGCAATTCTGAAGTCACTTGCTAAGA
ACGGTGTTGAAGTAATTGCTTCGGACTTTAAGAAAGGGGCTTCCGAGATGTTTATTTCTA
TAACTAAAGGATCTAAAGCTAAGATTAAATCATCATTCGGTGTTGCTCGTACCGATCAAA
TCGACAATCATGACTTTAAACAAACTGGTGCTAAACGACAGAATACAATTGCATCACGCG
GAATAAAATAGTTTATAAAACAGTTTACTTCTGGGTAGTAGTGTGATACTATTACCCTAT
CAAAACAAATGAGTGATTGGAGAATAAAATGAAACGCTGTGAATTAATTCGAAATGTTGC
TATTGCAATTTCTGCTTCCGCTTTTAGTTTTTCAATGTTTGTTGGATTTATATGTGGATT
ATTGACTACAGCAGAAAATGTTTTTTCACTTGTAGTAGCATTTTTAATTGGTTTAATCGC
TATTGTTATGGATAAAATTTCTAAAGGTGAATAAATGAATATTAACGAATATTATGTTTA
TGCTGGGGATTATGCAAATCCTTCTCATTTTGAAGGTAATTTAATACCTGATAGAGTTTT
TAATACTCCTTTTGAAGCGTGGAGTTGGGTTGAAAGTAAAAATGGATTTTCTTATCGTTA
TGTTGAAGTAACTGATTGGAAAGGAACTAAATATCCTAAAGAGCATTATTATGTAGACCC
ATCTAAAGTAAATTTTCTTTTGTTCGCAGGCGATAATTACTATCCTTGTGGTGGATATGA
TGATTTAATTGCGTATGCTGAAACAGAAGATGAATTACGTCAAATTATTGAAGACAATAA
ATCAAAACGATTTGGCGATAGTTTCGACTGGTGGCAAATCGTAAATGCCCATACACATAC
TATTGTTGATAAAGGTTAATAATGATTCTTTATGCGAAAGTATCATCCATTGAAAATGGA
TATAAATATGATCAAGAAGCAGCTAAAGCTTTGATTGATGATTATGGCATTTTAACATGT
TTTGAAGTTGAAAAGGTTTACATTGACCGTTCATCTTCTCAAGTTAAATTAGTGAAGGAA
GAACGTAAATTTAATACAGTAAATTTTGATTTCTTTATTGAAACAGAAAAAGGTCCTCTT
GAATATGATATTTTCAAGAATCCTTTGGGTCTTGAATGCATCGTAAATATGTATTATTAT
AAATGGTAAATATGCTTTAAGAATTATTTGTTATTATTAACTCACACCGCACTGATTAAT
ACCCTCTATCATCAAGGGTTCTTGTTTGAGAGCCTTTGCTAATAATTGGGAATTAGCCAA
GTTGGTAAGGCACTGGATTTTGATTCCAGGATGCAAAGGTTCAAGTCCTTTATTCCCAGC
GCGAGAATGGTCAAATTGGTAAAGGCACAGCACTTAAAATGCTGCGGAATGATTTCCTTG
TGGGTTCGAGTCCCACTTCTCGTACCAAAGTTTGCGGATATCGTATAATGGTATTACCTC
AGACTTCCAATCTGATGATGTGAGTTCGATTCTCATTATCCGCTAAAATTTAATTTACTC
CGTGTAGCTCAGTTTGGTAGAGCGCCTGATTTGGGATCAGGAGGTCCAAGGTTCAAATCC
TTGTATGGAGACTGGAGGCGTGGCAGAGTGGTTTAATGCACCGGTCTTGAAAACCGGCAG
TCGCTCCGGCGACTCATAGGTTCAAATCCTATCGCCTCCGTAATTTCGCTGATTTAGCTC
AGTAGGTAGAGCACCTCACTTGTAATGAGGACGTCGGCGGTTCGATTCCATCAATCAGCA
TATCAAGGCCCTGTAGCTGGAAGGTTCAAGCAAGCGACTCATAATCGCCAGATGGTGGTT
CAATTCCACCTAGGGCCATATTTTGGGAACATAAATACTCTTAGAAATAGGAGATATTTA
TGTTCCATTACACTTATAAAATAACAAATAAAATTAATAATAAAATCTATATAGGCGCTC
ATTCTACCGAAAATTTAGATGATGGATATATGGGTTCTGGTAAATTATTAAAAAGAGCCC
AAGACAAGTATGGTATAGAAAATTTCTCTAAAGAAATATTAGAGTTCTTTGACGATAAAA
CCTCTATGTTTGAGTCAGAAAAGAATATTGTGACTGAAGAGTTTTTAAAGCGCCCTGATG
TTTATAATTTGAAATTAGGTGGTGAAGGTGGTTGGGGCCATTTAAACACTAAAGAATATT
TGGATAAAAAGAAAGAAGCTTCCTTAAAAGGGGCTGCTGCTTTTAAAACTAGATTTGAGA
ACGACCCAGACCTTAAAGAAAAATATCGTAAAATGGGTTCAGAAAGGTTTAAACAGCTTT
GGGATAATCCTAAGTATAGAGAGAAATTTTTAAATACTAAATCTTTCTTGGGTAAGACCC
ATACTCCTGAAACTATTAATAAAATGAAAGAATCTCACGCCAGAAATAAACACCAACAGG
GCGAAAAGAATTCCCAATTTGGTATGGTGTGGATTCATTCATTAGATGAAAAAGTTTCTA
AACGTATTAAGAAAACTGACCCAATTCCAGAAGGATGGTTTAAAGGTCGAAAAATGAAAT
TTTAATTACGGGGCATAGCTCAATTGTATAGAGCAACGGACTTCTAATCCGTAGGTTGAA
GGTTAGAATCCTTCTGTCTCGACCAGACAAGAAGCCGGGTCGCTACCGGCAAGTCGTCGG
ACTGATGTTCCCTGTTTAAGGACTGGCATTAATAAATCTTTATGTTTTGTTAATGCCCCC
TTATATCACAGCAGAAACGGCGCACCGAATTATCGATTCGAGGAAATATCTTTGCCATAA
GCCGAGTAGCGTTTTTGACGGAACGTTCGGATATGGTCGAGATATGGCCTTTTAAAAATA
TTGAGTAGCGTCAACTACTTAATAACCGGGTTCGAATCCCGGCGTTTCGTACAAACACTT
GCCTTAGCAGGTGGAACCCCGACAAGGTTGCCGCAAGGCTTAGCCCCAGCCGAAAGGTTG
GGGCTTTTTGGTATCTAGGCCTTTCTGGACCTCTCTAGGCATCATTTAGTTTCTACCCTT
TATAATATATTATCCTATCCTTTAATTGCCCCTTCCCTCCCTAGAATTCCCTAAAACTTT
TTAAAAAATTTTTCACAAAATGGTTTACATCCCTGTTCTTCCATGGTACTATATCAATAT
CAACTACTGATACAGAAAACAACTTGGAGAATAAAAT

In another aspect, the invention provides an SF60B bacteriophage targeting a Shigella spp. bacteria. In certain embodiments, SF60B binds a molecule of OmpC. In certain embodiments, the SF60B bacteriophage has a genome comprising a nucleic acid sequence comprising SEQ ID NO: 7.

SF60B
(SEQ ID NO: 7)
AATTTAGCTTTAGCTTCTTTGTTATCATCAAGAGCCTTCAGCTCTGCAATATAATCTTTA
TCTATCATAATATTTCCTCAGTATAAATATAGATATATTTATTACATGGTATTTAGACAT
GACTGACATTAAAGTACATTTTTATGATTTTAGTCACGTGAGAATTGATTGTGAAGAAAG
TACATTTCATGAACTCCGTGATTTCTTTAGCTTTGAAGCTGATGGGTATCGTTTTAATCC
GAAATACAAATATGGCCACTGGGATGGACGAATCCGTCTTTTAGATTATAATCGTCTTCT
TCCATTCGGCTTAGTCGGGCAAATTAAAAAATTCTGTGATAACTTTGGCTATAAAGCCTG
GATTGACCCACAAATTAACGAAAAAGAAGAATTATCAAGAAAAGATTTTGATGAATGGCT
TTCTAAATTAGAAATCTATTCAGGAAATAAAAGAATTGAACCACACTGGTATCAAAAAGA
TGCAGTGTTCGAAGGATTAGTTAATCGTCGTAGAATTCTTAATCTTCCAACATCCGCCGG
TAAATCATTAATTCAAGCTTTGCTTGCGCGATATTATTTGGAAAATTATGAAGGTAAAAT
TCTTATCATTGTTCCAACAACTGCCTTAACAACTCAGATGGCTGATGACTTTGTTGACTA
TCGTTTATTCAGTCATGCAATGATAAAGAAAATTGGTGGCGGAGCATCAAAAGATGATAA
ATATAAAAATGATGCACCAGTCGTTGTTGGTACATGGCAAACTGTAGTAAAACAACCAAA
AGAATGGTTCTCACAGTTTGGAATGATGATGAATGATGAATGCCATCTTGCTACAGGAAA
AAGTATTTCATCTATTATATCAGGTTTAAATAACTGTATGTTTAAATTTGGTCTATCAGG
TTCATTACGTGATGGCAAAGCCAATATCATGCAGTATGTTGGAATGTTTGGTGAAATATT
TAAGCCAGTAACGACTTCTAAATTAATGGAAGATGGACAAGTAACTGAGCTAAAAATTAA
TAGTATTTTTCTTCGCTATCCCGATGAGTTCACTACTAAATTAAAGGGAAAAACTTACCA
AGAAGAAATAAAAATTATTACTGGGCTTAGTAAAAGAAATAAATGGATCGCTAAATTAGC
TATTAAACTTGCGCAAAAAGATGAAAACGCTTTTGTCATGTTTAAGCATGTATCACATGG
TAAAGCTATTTTCGATTTAATTAAAAATGAATACGATAAAGTTTATTACGTATCAGGAGA
AGTTGATACTGAAACACGCAACGTAATGAAAACCTTAGCTGAAAATGGTAAAGGAATAAT
TATAGTAGCTAGTTATGGTGTATTTTCTACTGGCATTTCAGTTAAAAATCTTCATCACGT
TGTTTTAGCGCACGGTGTTAAATCTAAAATTATCGTATTGCAAACAATAGGTCGCGTATT
ACGTAAGCATGGTTCTAAGACAATAGCAACAGTCTGGGACCTCATAGATGATTGTGGCGT
TAAGCCAAAATCTGCTAATACGAAAAAGAAATATGTTCATTTGAACTATCTTTTAAAACA
CGGCATTGATCGTATTCAGCGCTACGCAGATGAAAAATTTAATTACGTAATGAAAACAGT
TAATTTATAAGGGCTTCGGCCCTTTGGAGAAAAAGATGTTACTAGAATTTAAACAATTTC
TTTATGAAGCTTCTATTGATGAATTTATGGGTAAAATTGCCTCTTGTCAAACATTAGAAG
GTTTAGAAGAACTTGAAGCTTATTATAAGAAAAGAGTTAAAGAAACTGAATTAAAAGATA
CTGATGACATCTCTGTGAGAGATGCTTTGGCAGGAAAAAGAGCTGAATTAGAAGATTCAG
ACGATGAAGTAGAAGAAAGCTTTTAAATTAAAAAAGGCCCAACCAAAAAGGAAGGGCCAA
AACTATAGACTAAAGGTCACACTATAGCAAAAGTTGTGTTTCATTTAATTGTTCTTCCGA
ACTTTCTGAAACTGGTAGTTCTTTAATGTAATTATAGCAAGGCCCAGGATGTACAGGACC
TTTGTCTGTTTCAACAACCAATGCAGAATCGATTGGAGTTTTACAGACAACACAAATCTT
ATCTGACATGATTGTCTCCTCTGAATTATATATCTATTTATACAACTCTCATATGCATAT
CAATGCCCATATCTTTAGAATAAAAATATTCATCAAGATATCCGGCAAATTTTCCTTTAA
TATAAAGGACATCTTCACCACATGGATGATCGGCCAGGATACGAATATCCTGACGCTTAA
GATTATGCTTTTTCATTAAGAATTGAATTTCCGTTTCAAATTCTTCTTCATAATTAAAAG
CATCATCAATGCTATATCTCATTATTTTCCAGCCTCAAATGCCCGCATGTCTTGAATATG
CTTAATAGCAAATCCGCGTGATTTAATAGCATCAAGAGCTCCGCTACAGAAATCTAATAA
AATTCCCCAATATTGTAATGAAGTATCAACCTTTAAAACATCCTTATCCGCTGATAGAAC
TGTCTTCATTTCTGATTTCTCGTAACGATCCATACTAAATTCATCACCATCTCCTCGTCC
CGAGTAGTAGTCTAATTTAGCTTTAAGAGCAACTTTTTTCTGTGCATCAATTCTAAGCAT
TTCCTTTTTAATACTTGAATGCTTATTAAGCCATTTACTATATAACATCACATTATTAGC
TGCTTCATACTGTAATTTAGTCGAATCTATAAACACATCTTTCTTCAATTCTTCTTGAAG
ATCTTCTAATTTCATGTTGTTCTCTATTCAATTGTTATTGGATGGACTTAGATTCATTAT
ACCACGTTTATACGTGAAGCATTATACTCTATTACTGGAAGCCAGCTGCAGTTTTATCTG
CTCAATATCATCAGGGTTATCGATGACTGAAAAGCGTATTTCTACTATCAGAGTATAATC
ATCATAAACTGGTATCACATTAACTGCTAATTTGTCAATACGCGGCTCATAGTTTCTTAC
TGCGCTTTCAATGTTACGCTCAACCGTGTCAGCAGTAAGAGGAGTCATATTCTCAAAAAG
CTGATCAGATAAATCACATCCAAACTCAGGGTCAAACGGTCTTGACCCTTTTCTGGTTGT
AATAATTCCCAAAAGACTGTTTTTAATTGACCTTAATCCAAGGGATCTAGAAACATCTTT
GTCCCAGTCCATTTTCATTTCTGGGTCAATGTCAGAATAAAGCTTATTAATATTTGCCAT
TACAATAGCTCAAAGAATTCTTTGAGTCCTCTTATTACATGAGCGTGAGATTTTCCACAC
TCTGGACATTTAATTGGAACAGCCAAATAAACAGTAGGCTTTAAAAGCATATCTTTTATA
GCTACAATATCTGACTCTGTGATGATAGAATATAAATCTTCCAACTCTTTTTCATTTAAG
TCTTCAATTGGAATGCTTTCCCCGTTCGCGTGAATCGTTTCTATGCATGATACTATCATA
TGGGCTATATTTTTATCATCAAAAATTTTGGGGTAATGGAACTTAATTTTAATGTCACCT
AGTGTATACCAGAGGTCTTCTGGTGCATCTATTTGTGTATGTAATAGATTTATATGGGTT
GGTATTTCAGTTCCACAGGTGCACTTCCAGGAGTTTTCGTGATTAACTTCTCCAAGAGAA
TGCGCCCATAAATGAATCAGTAATAGTTCTGATTCTTGACGGTTTAAATCTTTTGCATTT
GTGCAGTCTTTGATTAGCTTTTTAACAATTTCTTCTACGGAACCGTTATTTTTAGCAGTA
ATAAGTTCTAGATACTCTTTAAGAGTAAATGCGCGACAATTGATTATTTTAGAACCAACT
CTCACGTCAAATTTATATTCATACATATTTAGCTCCTTTATTTATCATATTTATAAATAG
AATAAAAGGAGCATCTATGGCAAACATTATTCGTTGTAAATTACCAGATGGTGTTCATCG
TTTTAAACCATTTACGGTAGAAGATTATCGAGATTTTTTGTTAGTTCGAAACGATATAGA
ACATCGGTCACCACAAGAACAAAAAGAAGTAATTGCTGATTTAATTGCTGATTATTTTGG
AGACTATCCAAAGACTTGGCAACCATTTATATTTTTGCAGGTATTTGTAGGGTCAATAGG
TAAAACTAAAGTACCAGTCACATTTGTATGTCCAAAATGTAAAAAAGAAAAGACAGTTCC
ATTTGAAATATACCAAAAAGAATTAAAGGAGCCTGTTTTTGATGTAGCTAATGTTAAAAT
TAAATTAAAGTTTCCTTCTGAGTTTTATGAAAATAAAGCAAAGATGATTACTGAAAATAT
TCATTCTGTTCAAGTAGATGAAACTTGGTATGATTGGAAGGAAATTAGCGAATCAAGTCA
AATAGAACTTGTTGACGCCATCGAGATAGAAACATTAGAAAAAATTCTCGATTCAATGAA
TCCTATTAATTTAACATTGCACATGTCGTGTTGTAATAAGTATGTAAAAAAATACACTGA
TATAGTAGATGTGTTTAAGCTATTAGTTAACCCAGATGAAATATTTACTTTTTATCAAAT
TAATCACACACTCGTAAAAAGTAATTATAGCTTAAATTCAATAATGAAAATGATTCCTGC
CGAGCGCGGATTCGTATTAAAACTGATTGAGAAGGATAAACATCAATGAGTATGTTACAA
CGCCCTGGATATCCAAATCTCAGCGTTAAATTATTTGATAGCTACGACGCTTGGAGTAAT
AATAGATTTGTTGAATTAGCTGCTACTATTACCACATTAACTATGCGGGATTCTCTTTAT
GGACGAAATGAAGGAATGCTGCAGTTTTATGATTCTAAAAATATCCACACAAAAATGGAT
GGAAATGAAATAATTCAGATTTCTGTAGCTAATGCAAACGATATTAATAATGTTAAAACA
CGAATTTATGGATGCAAGCATTTTTCCGTGTCAGTAGATTCAAAAGGCGATAACATCATT
GCTATTGAATTAGGAACTATTCATTCTATAGAAAATCTTAAATTTGGTAGACCATTTTTC
CCTGATGCCGGTGAATCTATAAAGGAAATGCTTGGTGTCATTTATCAAGACCGCACATTA
TTAACTCCAGCAATAAATGCTATAAATGCTTATGTTCCTGATATTCCATGGACTAGTACA
TTTGAAAACTATTTGTCGTATGTTAGAGAAGTTGCTCTAGCTGTAGGAAGTGACAAATTT
GTATTTGTGTGGCAAGACATCATGGGAGTTAACATGATGGACTATGATATGATGATAAAT
CAAGAACCGTATCCAATGATTGTCGGTGAGCCATCTTTAATAGGTCAATTCATCCAAGAA
TTAAAATATCCATTAGCATATGATTTCGTTTGGTTAACTAAATCAAATCCTCACAAACGT
GACCCAATGAAAAACGCTACTATCTATGCGCATTCATTTTTAGATTCTTCATTGCCAATG
ATTACTACGGGAAAGGGAGAAAACTCTATTGTAGTGTCAAGGTCAGGTGCTTATTCTGAA
ATGACTTATAGGAATGGCTATGAAGAAGCTATTCGTCTTCAAACTATGGCACAATATGAC
GGCTATGCTAAATGTTCTACTGTCGGTAATTTTAACTTGACTCCTGGCGTTAAAATTATT
TTTAATGATAGTAAAAATCAATTTAAAACCGAATTTTATGTTGATGAAGTTATCCATGAA
TTATCAAATAATAATTCCGTAACTCATCTATATATGTTCACTAATGCAACGAAACTGGAA
ACAATAGACCCAGTTAAGGTTAAAAATGAATTTAAATCTGATACTACCACTGAAGAAAGT
AGTTCTTCCAATAAGTAATAAAGAAGTTTCTATTCCTAAAATGGGTCTTAAACATTATAA
TATTTTAAAAGATGTTAAAGGTCCTGATGAAAATTTAAAACTTCTTATTGATTCTATTTG
TCCGAATTTATCACCGGCAGAAGTTGATTTCGTTTCTATTCATTTATTGGAATTTAATGG
AAAGATTAAATCTCGTAAAGAAATAGATGGCTATACTTATGACATAAATGATGTTTATGT
ATGCCAAAGATTAGAGTTTCAATACCAAGGAAATACATTTTATTTTAGACCTCCTGGAAA
ATTTGAACAATTTTTAACGGTGAGCGATATGTTATCCAAATGCTTGCTTAAGGTCAATGA
TGAAGTTAAAGAAATTAATTTTCTTGAGATGCCAGCATTCGTTTTAAAATGGGCAAATGA
TATTTTTACAACTTTAGCAATTCCTGGCCCTAATGGTCCAATAACTGGAATTGGCAATAT
TATTGGATTATTTGAATGAAAAAGCCACAAGAAATGCAAACGATGCGTAGAAAAGTTATT
TCAGATAATAAACCAACACAGGAAGCGGCTAAATCCGCTTCCAACACTTTATCTGGGCTT
AATGACATTTCTACAAAATTGGATGATGCTCAAGCTGCTTCTGAATTAATAGCTCAAACT
GTCGAAGAAAAATCGAATGAAATAGTTGGAGCAATTGATAACGTTGAAAGCGCAGTGAGT
GATACTACTGCTGGTTCTGAGTTAATTGCCGAGACTGTTGAAATTGGCAACAATATTAAT
AAAGAAATCGGTGAATCACTCGGAAGCAAATTAGATAAATTAACAAGTTTACTAGAGCAA
AAAATCCAGACAGCTGGAATTCAACAGACGGGAACGAGTTTAGCAGTAGTAGAAAATGCT
ATTCCTGTTAAAGTCGTTGAGGATGATACAGCTGAATCTGTGGGTCCTTTATTACCGGCC
CCCGAAGCAGTTAATAATGATCCTGATGCTGATTTTTTCCCGACCCCTCAGCCAGTTGAG
CCAAAGCAAGAATCACCAGAAGAAAAACAGAAAAAAGAAGCATTTAACTTAAAATTATCT
CAAGCTTTAGATAAATTAACAAAGACAGTTGATTTTGGATTTAAGAAATCCATTTCAATT
ACTGATAAAATATCAAGCATGCTATTTAAGTATACCGTCAGTGCTGCTATTGAAGCTGCT
AAAATGACTGCAATGATATTGGCTGTTGTTGTTGGAATAGACCTGTTGATGGTTCATTTT
AAATACTGGTCAGATAAATTTTCAAAAGCCTGGGATTTATTTAATACTGACTTTACTAAA
TTCTCTAGCGAAACCGGAACATGGGGTCCTTTATTACAGAGCATCTTTGATTCTATTGAT
AAAATTAAACAACTTTGGGAAGCGGGAGATTGGGGTGGATTGACAGCAGCTATTGTTGAA
GGACTCGGAAGTGTTCTTTTTAATTTAGGTGAACTTATTCAATTAGGTATGGCTAAATTA
TCTGCAGCAATTCTTCGAGTCATTCCTGGTATGAAGGATACTGCTGATGAAGTAGAAGGA
AGAGCATTAGAAAATTTCCAAAATTCTACTGGAGCATCTCTCAATAAAGAAGACCAGGAA
AAAGTTGCAAATTATCAAGATAAACGAATGAATGGAGACCTTGGTCCGATAGCAAAAGGA
TTAGATAAAATTGCTAATTGGAAAACTCGTGCATCTAACTGGATTCGTGGTGTAGATAAT
AAAGAAGCACTGACTACTGACGAAGAACGTGCAGCAGAAGAAGAAAAATTAAAGCAGCTT
TCACCTGAAGAAAGAAAAAATGCTTTAATGAAGGCCAATGAAGCTCGTGCCGCGATGATT
CGTTTTGAAAAATATGCTGATTCAGCTGATATGAGTAAAGACTCAACGGTTAAATCAGTT
GAATCTGCCTATGAAGACCTTAAAAAACGGATGGATGACCCGGATTTAAATAATTCACCG
GCAGTTAAAAAAGAACTTGCGGCTAGATTTTCTAAAATTGATGCTACTTATCAAGAGCTC
AAGAAAAATCAACCTAATGCCAAACCTGAAACTTCGGCTAAATCACCAGAAGCGAAACAG
GTCCAAGTTATTGAAAAGAACAAAGCACAGCAAGCTCCTGTTCAACAAGCATCTCCTTCA
ATCAATAATACTAATAATGTTATTAAGAAAAATACTGTCGTTCATAATATGACACCTGTC
ACAAGCACAACTGCTCCTGGTGTATTTGATGCGACTGGAGTTAATTAAGGAATAATATGG
CAATTGTTAAAGAAATAACTGCTGATTTAATTAAAAAATCCGGTGAGACAATTTCAGCCG
GACAAAGCACTAAATCAGAAGTAGGAATTAAAACATACACTGCCCAGTTTCCAACTGGGC
GTGCTAGTGGTAATGACACTACAGGGGACTTCCAGGTAACAGATCTATATAAGAATGGAT
TATTATTTACTGCATACAATATGTCATCTAGGGATTCTGGAAGTCTTAGATCGATGAGAT
CTAACTACTCTTCTTCATCTTCGAGTATTTTAAGTACAGCTAGAAACACTATTAGTAGTA
CAGTATCAAAACTATCAAATGGATTAATATCAAATAATAATTCAGGAACAATAAGTAAAG
CTCCTGTCGCAAACATTCTTTTACCGAGATCTAAATCTGATGTTGATACATCATCACATA
GATTTAATGATGTTCAAGAAAGTCTTATCAGCAGAGGCGGAGGTACTGCTACTGGTGTGC
TAAGTAATGTTGCTTCAACCGCAGTATTTGGGGCACTGGAAAGTATAACACAAGGTATAA
TGGCTGATAATAATGAACAGATTTATACGACAGCCAGAAGTATGTATGGTGGTGCTGAAA
ATAGAACTAAAGTGTTTACGTGGGATTTGACTCCACGCTCAACAGAAGATTTAATGGCTA
TTATTAATATCTATCAATATTTTAACTATTTTTCTTATGGTGAAACGGGTAAATCTCAAT
ATGCCGCTGAAATAAAGGGGTATTTAGATGATTGGTATCGTTCTACGTTAATTGAACCTT
TATCTCCGGAAGACGCAGCTAAAAATAAAACACTATTTGAGAAAATGACATCGAGTTTAA
CTAACGTTCTAGTAGTTTCAAACCCGACGATTTGGATGGTGAAAAACTTTGGTGCAACAT
CTAAGTTTGATGGAAAAACGGAAATATTTGGTCCATGCCAAATACAGAGCATCAGATTTG
ATAAAACACCTAACGGTAACTTTAACGGATTAGCTATTGCACCAAATCTCCCTAGTACAT
TTACTCTCGAGATTACTATGAGAGAAATTATTACGTTAAACCGTGCTTCTTTATATGCGG
GGACTTTTTAATGTATTCTTTAGATGAATTTAATAATCAAGCAATAAATGCAGATTTCCA
ACGTAATAATATGTTTAGTTGCGTTTTTGCGACAACTCCATCAACTAAAAGCTCTTCGTT
GATAAGTTCAATTAGCAACTTTTCTTATAATAACTTGGGTCTAAATTCAGATTGGTTAGG
ATTAACCCAAGGTGATATTAATCAGGGAATTACCACGCTAATTACAGCTGGCACACAAAA
ACTGACAAGAAAATCAGGGGTCAGTAAATATCTTATTGGTGCCATGAGTCAACGTACAGT
TCAAAGTTTATTAGGCTCATTTACAGTTGGTACATATTTAATTGACTTCTTTAACATGGC
ATATAACTCATCTGGATTGATGATATACTCTGTAAAAATGCCAGAGAATAGATTATCCTA
TGAAACTGACTGGAACTACAACTCTCCTAATATTCGTATAACTGGGAGAGAATTAGATCC
TTTGGTTATTTCATTTAGAATGGATTCAGAATCGTGTAATTACCGTGCAATGCAAGACTG
GGTTAATGCTGTTCAAGACCCAGTAACTGGATTACGTGCTCTGCCACAAGATGTCGAGGC
AGATATTCAGGTTAATCTTCATGCTCGTAATGGATTACCTCATACTGCGGTGATGTTCAC
TGGATGCATTCCAATATCAGTGAGCGCTCCTGAGTTATCATATGATGGAGATAACCAAAT
AACTACATTTGATGTTACTTTTGCGTATAGAGTGATGCAGGCTGGAGCAGTTGATAGGCA
AGCTGCGCTTGAATGGCTTGAATCTGCTGCTATAAATGGCATTCAAAGCTCTTCTGGAAA
TAATGGAGGTGTTACTGAACTATCTAGTTCGCTTTCACGACTTAGTAGATTAGGTGGAAC
TGCAGGAAGCATTTCAAACATTAATACTATGACAGGGATTGTCAATTCGCAGAGTAAAAT
ATTAGGAGCAATATAACAATGGGGACCGAAAGGTCCCCATGTTTTTATTTACGGAATGAA
ATGAAAGCAGCAACTGAAGCAACTAAACTGTCTTCAATATAAACTTCAATTTTTACCGGA
GCTTCTGACTCAAATTTACCTGTTACTACGCCTTGGAAAATACTTTCAGTTTGTTCTGGC
TTTGAAAAATTTTCAGAAGGAAAAATTCCGAACTTTTTATCTGTTCCAAAAATTTTGACA
AATTCATCGTAAACTGCTTCATTAAAAGCATTATCAGCAGGAATAACGCCTTCAACTACA
AGTTCTTGACCTAAAAAGCGCAAAGAAGATTTCATTTTGTGTTCCTCATGTTATGTTAGT
AAGACTACTATAACACAACACGAGGGACTTGTAAACTACATTTTGAACTTTTTAGACATT
CCGTTAATGTTAACCAAAGAAGCTAAGATTTGAGAAACTTCGTTTTCCTCTTCTTTTGAT
TCTTCTACAAGGAATTGATTAAATCCAGCCATTGGACGAACTTCACCAGTTTCCATCAGA
TGGTCGCCATCATATACTGATTCTTCTAATTGTTCAGAAGGAGGAACGATTGCACCTTCG
ATTAGATACTTGTTATAAGCAGTTTCTTTCATAAAGTAAGTACTTACTTTATTAATCTTT
AGTAAAATTCCACGTGGTAGAATAACTTCAGCTTCTGATGGAAATCCTGATAAATGACCT
GGAACGATAACTTTGATTCGCTCAGCTCCTTTAATTACCATACCTACATTAGTTTCAGCG
TCTTCATTCGCATATGTAGAATGACTACCCATATGCATTAAATCTTCAGCATCAACTGAA
CCTTCTCCTTCACCAGAAAATACTGCTCCGGAATCATCTAAGGCCATATAATTTTTACCA
TATTCACCGAAGATATTTGGCTTGAGTGAAGTAGATACAAAGTTCTTGAAATAGAACATT
TTATTTTCAATGTTATGACGCAAAGTTTTAAAGGTAACTTCTTGCCCACGATAAAGAGTA
GTTCCTGGAGGAAGAACTGAACCTTTAGAAAATGCAGAATCCAGCGTCTCAATATGTTTA
ATAGCTCGTTCCATATATTCTTCACGGGTAGAATCAGGTTTACCTAAAAGGAATGAGTTC
ATTTCAACATATGAATCTGCACAATAATCTTCAATTACATCAGATTCAGCCTGAGTTAAT
CCTTTTGGAGTTACTTCAGTACTTGCATATTCATATGCCTGAGCGGCAGATTCATCAATT
AATTTATAAATTCCATTCATGACAAATTGAATAATAGCTGTCTTTTCATCTGGTGTAGCT
TTAGAACCTTTAACTAAATCATTGACAATTTTCTTAATAACATCAAAGCTATTAGAAATA
TTAATGTCAGCAAGATCGCTTAAAATTTTATCAGTTTTATAATCAAAGAATTCTTTATTC
TTTAAATATTCTTGAGAACCTTGATATGTTATATCGCTCTGCAGCTTGATAGATGGATCG
CGTAAACGTGCATGGGCCAATGCAATATGATTTCGGAAGCTTTCGAGTTCATAATCACTA
GATTTAAAACGAGAAGCACTTCTCATACCACCGATAGCTCCTTTGCTAAAGTTTGAACCA
ACCTGAATAACGCCAGAGTTCATTGGTCCTTCTGCTTTATATACCGGAACTTTAGATTCA
AACTCTTGATATTTCTTAGCAGCGAAGCTATCACCCTGAAGTGAAGCATCAACAGTAGAA
TATTGAGCTTGTGCTGCTAATCGACGACTAATTTTAGTTTTCTGAATAACGGCTTGATCC
GTGCGCTTATCATTTTCTAAAGCAATAGAAGCAGCAACAGCAACAGCTTTAGGAACAGCA
TTACCTGTTTTAACGTCTACATAAACATCACCAACCTTTGATTCGACTTTTGTGAATAAC
TCGGTTGAAATAGGAGGTACACCTGGAATGTCTTCTAAATTGGCATTTTTACGGTAAACC
ATAATATATGCGTACTTTTTATCATAATCCCAGATTTCCTTTAAAAGAACATATCGACCT
GCTCCTTTAGTACGTACTAAACGGTCAGCAATAACTTGCATTTGACGAGCTGCGCCATTT
GTTTTACTTTTTAGTACGCGGAATAGGCATGCATCAATTTTATACTGCTTCATTGTTTGA
AGAGCAGTATCAAAAACAGCATTAATGACGCCAATTGGATTTCCACCCAAGTTTTTAAGC
TTAACTAATGATAGTTTCTCATTAACACCTATCATTACGATATGCATCATTTTGTCGCCT
GGCTTAACGTTTTTATTCGTATCACCGCCAGAGGTGTATGTACAAAATCTAAATCCAGGA
AGCACGCTTTCTTCACCTGCTTGAATAGCAAAAATTTGAGGTATTTTATGCTTAGGATTT
AAGTTAACAACCGGCGCCGAAGCGCCGTCAAATAATTCTGTAATAAGTTCCATGATTTAT
CCTTGAACGAACTTGTAAGGCATGTTTGTAATATCTATGCAAGACGCAATAATTCCAAGA
GATGATTCTACTTTCTGTTTAACATTTGATGAAACAAATGATGCAAAGTCAACTCTGTCT
TCTATTTCAATATCACCTGTCATTGCAACTACTTCACCGGTTTCCATTAAATGGTCTCCG
TCATAGATTACCGATTCGGTGAGCTCTTCCGTGGTCATAACTTCAGCTTGAATGAGTTTT
GTATTATTAGATGTTGTTCCGTCATTATTAGAAGCATCAGTTATTTTATTAACCTTGACC
ATCAATCCGCGTGGTAGAATAACTTCAGCTTCTGTTGCTATTCCGAGACTTCCTGGATAT
ACAACATTAACTTTATGAGCTCCATCAATTGCCCATCCTATTTGAACTTTAACGTATTCA
GGATTTTCTTTATACGCTCTTATTTCGTTTGGATTAATAGTTATTCCTTCTTCATTTTTA
TCAACTGTCAATTCATTACGAGCTTCTGGTTCTAAAAGACCAATACCAGCATGTGTAATT
CCAAAACGTCCAAAAATGATAGGAGTTAAAGAAGTAGATACAAAATTTCTGAAGTAGAAC
ACTTTATTTTTAACTAACGCTTCATATATAGGAGCAGTCATACTTTGAGCACGATAAACT
GTAATGCCTTCTGGTATACGGTCACCATTTTCAAAAGCTGAATCTAAGTTATTTATAGCA
GATTCAATTTCTTTTTCACTCATTACATCGTATCGTTCTGGATTGTAACGACCTAAAAGA
AGATTATTGATGTCAGTATATCCTGAACCGCAATATTCTCTAATGCCACGCTTTTGTGAT
GGAGTATATTTAGAACCATCGCGTGCAATAGTAATATCAATCATTGGACCAGCACCAATT
CCAGAAAAACTGTAAATCATTCGTTTGAGGAATTTAGTTTTTGCTGCGTTCCAGGAATTT
TTAGTTATTTCTTCCCAGACTTCATCTGGTTGTCCTTTACGATTTTTATTATGATTAGCA
AGCCATTTATCTTTATAATCTTCAAAGAATTGATTTACAGCTGCTGCGTATTTCTCTAAA
CATTTTATTGAAGTAGTTGGTTCATTTTTAATTGCATTAAAAATTACTTCTTTAATTTTT
TCTGAAACTTCACCGACTTGACTCATTGGAAAGAATTTATAATCATAGTGAAGTTCATCT
GCCGTTCCTTCAGCAATAATTTTTGCTGCCTTTGTTTTAGAAGCTAATGAATTAATTCCA
AGCTTCATTTCTTCTGCTTCGGGAATTAAAGGTGCAGTAGCTGGTTTATTAAAATTAGCT
GCTGTAGATTCATACTTCTGGAATAATTCACTTTCAAATCTAGAAGATTCCAATGACTGA
CTTTGTGCAATTGCTCTGCGAGAAATTTTAACCTTAACGATTACAGCTTGGTCAGAACGT
TTATCATTTTCTTGCGCAATAGATGCAGCAATTGCCTCATTTTTGGTTACTTGAGCTCCA
GTATCTTTATTAATATAAACATCACCGACCTTCGATTCAACTTTAGTAAAGAGTTCAGTG
CTAATTTCTGGAACTCCTGGAATGTCTTCTAGTGATACATTTTTACGATGTATAAGAATA
TATGCATACTTTTTATCATAATCCCAGAGTTCCTTAAGAAGGACGTATCTGCCACCTGAA
CGACTGCGGATGAGTCTATCAGCAATAAGTTGAATTTGTCGAGCTTGACCGGCGGTTTTA
GACTTAAGAATACGAAGCATGCAGGCATCAATTTTATATTGACGCATAGTTTGCATTGCA
ACAGTAAAAACTGAATTAATATAATTAATTGGGCTTGGACCAAGACCTTTTAATTTAGCA
ATTGAACCTTTAGCAGTTAATGTAAAAGGAACAATATGCATCATTTTATCGCCCATCTTT
AAATCGCGATTAGTATCACCGCCAGATGTATAGGTACATAAACGAAAGCCTGGTTGTTCA
ATTGCGTCATCAACATGAACTGAAAAAATTTGTGGTATTTTCTTCTTTGGATTTAAGTTT
GTAATCGGAAGAGTAGTATCTTCGTCAAATAATTCTGTAATAAGTTCCATCATAGCCTCT
CTAGTGTTTATTCTATTTATAAAATTAAAGGCCCGAAGGCCTTTAGTAATCTAGTGGTAA
GAGAGTACGATATATTTCAAACTTTGGTCCTTTTTCATAGGCATCAAATGTTTCCGTGAA
TTTATTATAAGCATATGCATCTACAAATTCGAGAATGACTTGTGATACAGAAGTAGAAAC
ATCTCCACCTTCTTTTTGAGCAATGACAATTGTTTCTAAGTAAGCTTTCATAGACCAGTT
ACCTCATGAAAATCACCAAATACATCCTCGAATGTATTAGCTTTAGTTTTATCTTCACGT
AAACGAATCGCAATCGGAAGAAATAATTTAACGTAATCAGTGCGGCCATCAGATTTTAAC
CAACCGTTGCATTCGCACTCTAGAATTTTTCCAATATAATAATTTTGGTTTTCCATAATG
CGAGTACGGTCAAGTTCATGCGATTTTACGCCGGCTTTATCTTTTAAGCCTGAACCAGCA
TTTACTTTAATTTTTCCACACTCTGACTCAAGAATAAATCCGCCTGCTTTAGTAGGGTCT
TTACGGTGAGGATAAATTCCTACAATTTTTAAATCAACATCAATTACTTCTTTAAATTTA
TAAAGATTTTTTGAACGAGCATTTTCCCATAATCCATCAACGTTTTTGAGAATAATACCT
TCAAGACCTTGGTCAATATACTTTTTATAAATTACCTTAGCTTCATCTAGGTTATTTACT
ACCTGGTTTTCAATTAAAATTACTTTATCATAGCCTGGTGTCATTTGTTCTAGTTTAGAA
AAACGTACATCATATTTCAAACGAAATGCAGGAAGACCATATATTTCTACTAATGGAACA
TAATCCCAGACCTGAAACTTCATGCATTGAGCTTCTTTTTCTGAAATAGTTCCCCTTAAA
GATTTATTAGCGATTCCATTAGAAGTAGTACGTGATTCAGCTACTTCGGCGAATTCTTTA
GCTTTACTAATTTCAGGATAAGCATCAAAAAGAAAATCTAGGCCTTCTGGTTCCTTTTCA
ACTTGCTCATGGTATACCAATTCGCCATCAATTAACACACCTTCTGGATGAATCTGGCGG
GCTTCAGCGGTCATTTTGATTAACTCTTCTTTGAGAAGGTCTAATCCTAGGTATTCATTA
CCAGCTCGTGATAAAAGACGAACATCATCTAATTCATCACCTCTGACTTCAGCAAAGCAC
CGAGCTCCATCAGCTTTTAATTGGGCAAAGGCTGGAAATTTGATATTCTTATTAATGCCT
TTTTCATCATAAGAACTTGCCAGCATTTGAGGTTGTTCAGGAATTAAACCTGGCCAAACT
TTGTTTGCAATAGATACTGAAGCACCGCATTCAAGGTCTCGCATCATCACCCGACGCAAA
ACTTCAACATCATCTTTTTTACCGTCAGCAATATATCCAGTTAATTCCTCAATTGCCGCA
TTTCCAGTCAATTTCCGAGTAGCTAACGTGAATTCAATGAAGTCGAGCATATCTGCAAGA
GTCAACATTCCAAAACTCTGGGTAGCAATACCAGGTTTAGGCCATTTCTTGATATAATAC
TGTAACCCACGAGAATAAGTCAGACGATATACTCGTTTAAGCAATTCATTATCTTTATTC
TTTTCAAGAATTGCTTGCTTCTGCTTAGTTGAACCAATAGATGCTATTTCGTTCAGAATT
TTAAGAATCATTATTCATCCTTCAGAGTTTGGTTTACAACTCTATTATAAATCAATTCAT
CATTAAGCTCAGTCAAAGACCTGTGGTACGTGGTTCTAACTTTATTTCCTTGCATCCAGT
GCTTGATATAAATAAAACCTTGCTCTACACATTTTTTAAAAATTCGTTCGTCTTTTTGAG
CTCGGAATTCTGGATTGCATCTAAAAAATTGATTTACGTGACCGTAATCACGTGTAGTAT
TACCTTCATTTTCATAAATAGTATGAACGACAAACATTAGAATGCTCCTTGGAAAATATT
ATCACCACAAGTAGGTCTATTATACAAATACTCTATGCCACCAGGTTTAATATAGTTCCA
TGTCTGAAATGGATGTGTCTGATATGGATGGTATGGATGGTATAGATGATTATAAGGATT
AAATCCAGGAGTTCTCCAGGTAGTATTCCAAGGAAAAGAATCGTTTTTGATCATCTTCTC
AATTACATCTTTTATAACTTTTTCACTATCAAAACTTTCTTTATTTTCCTTCGATGGAAA
AAGCTTATTCTCTACATCATTCCATGTATAAACTCGCTGTGCAGTTTTTGGAATACTGTC
ACGCTCCCCTCGAGCCATCCAATAAACAGGAACATTTAATATTTCACTCGCATGATCGCA
ATGGTGAGCGAGATCGTCAATATAACAAATTACGTTATATTTCTCTTTTGCCTTTTTGAA
CAACTCTTCTTTTGAAGAATCATGACCACACATCAGTACTTCTGAGAAGGCACCAGGAAA
AAGAGCGTTCAAATTAAATTGACGATTCAGCAGAGCGTCAATAGAATCACCTAATGCTGT
AACAGCTACAAAATTATAATCTTCTTTTAATTTGTTAATTACACACAGAGCATCTTTATA
TGGAGACAAGTAACGAATAAAATCTGATCGATTGTATTTTTCAATTAGCTTGACACCAAG
TTCTTCATCACAATTAAAGAGTTTACCTGGAGAAATAAATTTCTCATCTTGGATCATTTT
TAAAATATGTTCTAACGGAAGATTATATTTCTGAGCAAAATAAGGAAGGCCTGATTGCCA
GCTCAAACATACTCCGTCAATATCAGTTAAAATAGTTGGCTTCATAGAGAGTCTCTTAAT
AGGTTTAACACATCAATAAATTCAGCTTCGGTTAGTATTGTATCATCTTTTGTTAGACCA
CTAGCCATGCTGTGCTTCAAAACTTTTCCTTTCGAGGCTTGTAATGCATCACGAAAGCCC
TTGTTTTGAATCGCTGCTTCAAAATATGCATTTGTGTATAATTCTTTCCACGCCGGGGAG
TATCTTGAAAACGGAACTCCAAGCCAGAAGAGGGTCCCACGGTCCTGAGCTCTAGCATAA
GACCTTCCAGCTTGTTGAGCGGCAAGCCCGGATAACCCAAATATACGACGTTGTTGTTCA
ACGTTTTTCACCTTGCATCCTTGGAGGAATCCTTCAAGACCTCCAAATTGAATACCATCC
ATAACGAAAGGCCATTTGGCGAAATTACTTAATGCACATGATGGCCACCTAAAATTACTT
CTAATCTCTAACTCAGACATTTTCAATGCTTATAATTTCAACATCAGCCCAATGACCATA
GCAAGGAAGACGAAATTCAACTGGCCAGTTAGGGTCCTTTTCTAATATAATAGACTCAAC
TCGTGGTTCTTCATGTTCATCAGTATATGGATTTTTATCTGTTACTTTATATGTGACCTT
AATGTACTGAATTCCAAAAATCTTATTAATTATATTCATACTAATTCCTTTAATCCGTAA
ATAGGAGATAATTCATCACCCATGCGAAGGTCTTCATTTCCATCTACCCAGGAAACAATA
TAAGCCTCTTTTATTTGAATACCACTCCATTTAAATGGAGGTAGAACCTTAGAAATTAAT
CCAGGTATACCAACTCCCTTTAATTCAACAGTTTGGCCTAAAAAGAATTTCATTAGAACC
TCATCTGAAAACCGTGCGATTTAACATTACCGCCACCAATATCAGAGATATTAATTTCGC
GCGCAATTGAAGGGTCAATGTCAATTTCACGATTAAGTTTAGTGATAGCTAAAGTATCGC
GCCCATTTACAGTACGAAACTCTAAAGGACAAACCACATCAACGTAGTTTTTAATCGATT
CGCTAGTAGGTTGCAAATGTGCAGGAAGGTCTTTAGGTGCCTTTGAGAAAACAACTTCAC
AGAAATTTTTGCGGGTATCAAAATAAGTAGTCATAAACATAATATTTTCCTCAATAGGGG
GCCGAAGCCCCTGTTTTATTTTAAATATTAAATTCGTTAAGAACTACATCAAAGATTGCT
TCAAGATGCTCAGGTTTAGCTCTGTTACTCAGAATATGACGAATCCAAGTTTTAACTAAG
AGTTTACGATTAGCACCATTCCAGCAAGGATGAGTCCCTAAATCACGTTGACGAAAATCA
TCATCCAGAGCGATTTTGAAGTTGGAACCTTCCATCGTGATGGAAACTGTAATACCATTT
TCAAATCGCATATAAACGTAGTTAGGAGTCATATACTGTTCAATTTCGCATACTGATCCA
TTTTGATGTTTCCAAAGGCAGATAGTATCAATAGAACCTGCAATACCGTTAGAAACATAT
TTACGTTCAAAGTTGATGTAGTTCATTTTATTCTCCAAGATGTTTAATTGCGGTACAGGT
ATATAATATCATATCCTGCACCAAAGTAAACAATTATTTTACTACTTTCCAATGCTTCAT
ATCAAGTTTACCAACTTTTTTCATCTTCTCAATTAAGCGTTCGGCACGTTGGCGAGCTGT
AACATAATACCATTCACCTAATTCATTTTGTTCAATTTTTCCAACGATTACTGTATTCAA
TTCATAAATCCAACCAGTAAAGAAATTATGAACTTGAATTGTAAAGGTGAAATCTGTTCC
CATACCTTCTGTTGTTTCTACTTCAATAATATCACCTTCAACTGCCATTAAGAACCACAT
AGTTTCATCATATTTACCATTGAAGCATTTAGTTTTAACTGCAGCGTTCAGATTAATCGT
TTTCATTTTATTCTCCTTTGTTTGTGTAAGATAATACTATCACAAAGGAACCATACTGTA
AACAACTTTGTGCAATCTTTGGAAAATAAAAAAGGACTCCCGAAGGAGTCCTCAACTTAT
GCTTTCTGCTTACCAAAACGAGAAGCATCATCTCGAAGAACCGCACGTGCTCGGCGCATG
ATCTTCTCAACAGTTTGATTGATACGAGAGTTCGACCCACGCTTGTAGCCAGCGCGTTTA
GAATCACCAACTTTCTTTTCAACTGCTTTCTTTGCTTTAGCTTGTTTTGCCATTATAAAT
TCTCTTTTAAATGAAAATGCAGGACTTATTGGCATTGCCTGCGAAAGCCCTCAAGGGGTA
CTTAGGTTTTGAATATTTACCGACAGGATAACCATAAACCTCGTCAACATTCAAGAGGTA
CACCGTAAAACAAAGTTCGACGGCTCCTCGTTTTAACTTCACACTAAGGCAGTGACTCTC
CAATAAATTACTTCAGTGTTACCACAAAGTGACGAACTGCTTTTCGTGCGGCAGAAGCCA
GAGGCTTAGCATATTTAAGTTCATCTTTTTCCTGAAGCTCGGCAGCTAATGCAGTTTGAG
CAGGATTCAGATGTTTGAAATAACGCAGGATTTCAAGAGCTTCGGCTTCAACATCAATAG
ATGCGCCATAGTTTTCGTGACCATTATTCCATGCGTTTCGTTGCAGTTCAAGAGCGTGTT
GTAATTGTTTAATCATTTAAAAATTCTCGTTAGAGATTAAAACTCGGTAGTCACGTTCTT
CTGAATTTCATCTTCTTTCGACAGATCTCTCAGTTGTAGACTACCACATAGAATTGAAAC
TCAATTCATATTATTATTTATATCACTTATAAAGACACGGAATAGCTTTATAATGACAGG
TAACGAATTTTTGTTTAATTTCTTTTGGCTGCTTAAGACCCAGAGCTACAAAAGGATGCG
GAACGTTTCGAATTTGACCAACTGGAAGAGAAGTCAAATCACCGACTTCGCAAAAACCTT
CAGGAACATCAGGACCAACAGAGTGAACTACACATAGTTCAGGAACTTCACCTTGAACAC
GTTTACCGATAATAAGTCCTGATTCTGTAACTTCTTCATCACCGGCTTGTGCAGGTTCAG
AAACTAAAATAACATATTCACCGACAGCACGAATTGGTAGCTGTTGTACTTCAGACATCG
TTTTTCCTTTTTGTTAACAGATGAATTAATAATAACAAATAATTCTTAAAGCATTTATTT
ACCAATAAATTGAAGCAAATGCTCAACTTTCATACCATTAACGGAAATCAATTTGTCAAT
AGAAAAACCTCGCCATGCACCAAGTTCAACATCAAATACTGGAATCATGTCAGTAGATTC
TTTCCGAGTAGATTCAGTCAATTTACCAGTTTGCATGGTTGGCATAAAGTCTGCATCACG
AGTACCTTTCATGGTACGAATAGTACCATCAGACTTTTCAAAAACTACGTTTGAAACACC
CATGGACAGTTTGGTTTTCAAAATTTCGCGAATTGCTACTTTCTGCTCAGTTGTCAGTTT
CATTTATTTACCTATCACAGTTTTAATATGAGTTGTTCCACGTTCTTTAAGGGTGGAAAG
TAATTTTTGGCATTTTTCTAAATCAGATTTCCAACTATATGGTCTATCAATACTAGTCCA
ATCAGTTTTATAATACTGTTTCCATTTAGAGAAAAAATATTTCTTATACTCTACAGCAAA
CGAGATGTTCTCATTAGAATAAGAACTAATTGCTGTAAGCTTTACCAAACGAAATTTCAT
TATTCACCACAGAATTCGTTGATATTTTCCCAGTTTAACTTATTCAAGTTTTTCTTAGGA
ACATTAAACACTTCAATACCTGCATTTCGCAGAATATCGTCCCAGCCAGGTTTATTTTTA
TCGTATGTTTCGCAATAAACCAGCTTTTTAATACCAGATTGAGCTATCGCTTTTGCACAA
TCTGGACAAGGAGAAAGTGTTACATACATAGTAGCACCTTCAATAGAAGAACCATTTCGT
GCAGCAAACAAAATTGCATTTAGTTCAGCATGAATTTCATTTTTAGACGACCAATCAGAG
TGAGCGCTGCGATATTCTTTTGCCAAAACAAATCGATCAGTTAAACCAAATGATACGTAT
TCTGGCTTATGAGCTTGAATAATAGTATGTTTAGGCTTATTCAGCAACCAGCCTTTCTCG
GCAGCATAATCACAGCAGTTCACACCACCTGCAGGTGAACCATTATACCCAGTAGAAATA
ATACGTCCATTCTTTTCAATTACTGCTCCTACCTTCCAGGAGCAACATTTTGATTCTTGT
GATACTAAATATGCAATTTGAAGTACTGTACTCGCTTTCATTTCATAATCACCAGATAAG
CAGATTTAGCAGTTTCAACACGATAAATTTCGTGACGAAGTTTAGTTATACTTTTAATAA
CAGAACTAATTATATTCTGCCCATCTTTAAAGCGGTTTTTCTTATCGATAAAAATTGCGC
CAATCATCTTTTTGTGAAGCTCAACTGGATACTTCGTCACAATAATAGCATCATACACAG
AAGGATGAATACTATTCACTAGAGTATCATTCATTAAAGTTATTCTAATGAACTGTGCTG
TTTCAGAATCAAGCGCTCTATGATCGCCAGTATCATTTTCAAGACAATTATCAATTATAT
CAGTTAAATTCATCATAGTACGCCATACACCCTTTGTGCTTCAACTAATCCATCAAAATC
CAGTTTAAGATGCGATATTTGATCGCCATCACCTGGATTCACAATTACTAACACTGAACG
AGGAGTTTCGGTAATAACACGAACCGATGTTTCAGGAAATCGTTCAGAAACCTTATTTAC
TAATTCCTGTGCAAATAGTTTAACTTTTTCTTGGAATTCTTTAACAGTAATTGGTTTTTC
ACTTAGCATGTTGATCCAAGCTCCTTACTTTCAACGTTTTTAACTAATTTGACAGAAACT
AATTCAGCATCTTGGGTAATCATTACCGGGCCTTCTTCATATTCTTCAAGATATTCTAGA
GCTTTCTTTTTAGCTTCTTCCTTCGTATCAGTTTGAGCATAGATATAGAACATACAACCA
GTAATACCTACTACTTTCCACTTAATTTCTTTATCAGTTTTAATTTCAAATTTACTGACA
GAATCATCTTTTTGAACTTCTTCGAAAAAAGAATATTCTAACATATAAGCAATTGAAAAT
AACGGGCTATCTTTATAGGATTCGCCCATTACATCAATGGCTTTATACCAGCAGTCATCA
ATTTCGCGTTTAATGCTAATAACATTCTTTGCAGGGTCAATTTTTACTTTAAATGAATTT
TCATCAATAAACTGGGCAATTCGTTTATTAGCCCCGCAAGTTTCATTAAAAATATCTTTA
TACCCAGTTTTAAAGCGATAAACCTTATCTTCTTCAAACCATTCGCTCATTTCAATTTCC
TCAATTGTTTTGGTAGAGCTATAATATCACAACTCTACCGTAAAGTAAACCATTAAATCG
CTTTGAATTCTGATGTCTGTGATTCAAAGCGAATATCGCCTTTGATGACTAGCTCAGCAT
CAAGACCGAATACGACAATGATATGCGCGGAACCTGGATACAGCGTAATTGCAATAGAAT
CCACTTGGTCTGGAAGCAAAGCGTTCAATACATGAGTCACTTGAGCATGAATTCGAAGCT
CAGCTGCGTTATCAAGTTTTTCAAACATATTATTAGCGATAATTTGGCTAAACACTACTT
CTACGATTTTAGAGTAAGTCGGAAACATATTTACCTCACATAATTTTCTTCAAGCCAATT
AATAATACCCAAGTTTCATGATGTAAAGCCATTAAAAATCTCCCGAAGCGACTTGCCAGC
ATTCAATACCGATGCGACGCCACATTTCAACTACTTGAGTTCGGTCATCAATAGCTAATT
TCACATCAAAATGTGGTGCGATGTGTTTCCAGAAAATTTCTTCTTTAACTACATCATCTT
TACGGGTATCGCCTTGTTCGCGTTGACATTGCATAACTAATGGAACGCCAGCAATATCCT
CAACCCATTTACGGGTCATACGATAATATTTCGTCGGGTCTTCTTCAGTTCCACTTTCAC
GGCCTGAAACGACTACGATTTGATAACCCATAAAAGCACACATCTTAGACAGTTCAACAA
CCATAGGATTGATAACATCGGTATCGCATTTTTCAAGGTCATAAGGACCACGACCATTCA
TTTTGGCTAGTGTACCATCGACATCAAAAATAACTGCTTTTGGTTTACCAGGAGTCCCTT
TGTATACCGGAAGACCGAGATACTCTCGCATTCTTTTATACATTGAGCGTAAAACATCAA
TTGGCACTGCTTTAGTTCCACGCTTTTCGTTACGTTTAACTAATTCTGTCCAAGGAACAT
CAAACACTTTATATTCAATTTGATGGCCAAGCTCTTTGGCAAACTCTTCCCAGACCTTAC
GTCGTTCAGGATTCAGATTAGTATCTGAAACAATTACACCCTTCGTTGCGTCTTGGCAGA
GAATCATGTTAGCAACATCATGTTGCATGTAAGTTACGATGCCTTCTTTCTTTTTGGTAT
ACTTATACTCATCACGTTCTTCATGCGCCATAATAGATTGGCGATAGTCATCACGATTGA
TATTATAAAACCCGGGATTCTTAGCAATAAATTCACGAGCCCAAGTGCTCTTACCAGAAC
CAGGACAGCCAATAGTCAAAATAATCTTTTTCATCATTTAATTCCTAAGAAAACTTCAAG
AATACGAATATTGTGCTCACGCCGTCCTTTGTACAATTCAGCTGTTGATTGATTCGCTGA
TTTACTCTGAACATTAGATGAAATAAATTTTATCATGTGATTTTTAAGCTGATACATATC
AAGTCCACGAGCTTTCGCTTCTTTTCGCAGGGCTTTACCAGCATCATCTAAAGCTTTAGC
AGGGTCTTCATCATTTAATACTATTCCACGATCCAAATCCATGTATACTGCGCCATGACA
TGCATCAATGTAACGATCCGAGCATTCAATATAACGTTCTAGCAGGGTTTTCATTTATTT
TTCTCAACCAATGATTGAATATAATCATGCAGGTCTTTAGATGCTTTACCCCACTTATTT
TGATATTCATTTTTGAGATTAGCACGTGATTGAGCTAATAAAACATCATTAGTTGGAGGT
AAAGATTCTAACCGCTGAATCTGGCGTCCATAAATCATTGCAGCCATCTCGGATTCATAA
ATCAATCCTTTGAGATGTTCAAATTGATGCCATGAAATCATTTGCATTTATCCTCTTTTA
ACTCTTGACGATAATAACATATCATAGTTTTCTGATCATGTACATATCGTTTTACATCAT
TAAGCCAAATACGAAATTCCTGGGAATCTTCAAATGGCATACCGACCCAAGCTTTACCAT
CAATAACTTTAACTTGCCAAGATAGTTTAGCTTCGTCATATGACTTTATTTGTACAGGCC
AATTAGGATGAACTGTTTCTTTCTTTACTTCTAGAGGCTTTGTCGAACAACCAACTAGAA
GACCAATAGATAATATTACTGCTGATAGTTTAATCATTTAGAAAGGTCCTGGATGTCTTC
TGCGAACTTGTTGAAGGAGTTGTTGATTTGTTTTTCAACCAATCCTGGCTTATGAGCCAC
CACATCCGCCTTCTTTGCATCTTTGCGCAGTTTTTCATTTTCACGCTCAATAGCAGCAAT
TGCCTCACGATTTTTATTATTCATCGCATCAATATAATTATACTGAATTCGCAAATTATT
TAATGCTAAGGCGTTTTCATTGGCCGTTTTTGTAATTTCAGTAACTGATGTTTCTAATCT
TTCTACCTTATTTTTTAAAATAATAGATGTTCCGCCAAATGCTATTACAAGTAATAGCAA
TCCAGCTGTTGTATTACTTAATTGCATAAAGTTTTAATAACCTCTATAATATCGTCTTGA
GAAAGACCGTTAATTAAAATATGATGTTCAGCTGGAGATTTAGAAATTTTAAAGCATGCC
GCAACATCTTCTGCCATATCCGATGCGCTACGATTTGGATTACTAATTCCAAGACGATGT
TTTCCCGTCAAAGGATTGACGATGATATAGCATTTGCAGCTATTGATATGAACATTAGGT
TGAGTCTGATTAATGAACACTTCACAATCATACTTAGCGAGTTGATTTTCTAAAAAGACT
TTCATCTCCTCAACCGCATCAGGAAGCATATCACGGGCTTGCTCAAGACGACGATTTCGA
TATTCTTTAATGGTCGTTTTCCGCTTGACTTGTTTAGCTAAATCTTTCTTAAGATCGGTG
ATATATCCAACTCGACGATTTCCTTTAAATACAGAAATCCCATCCGTAGTATCACCGTAT
GCTTCAACGACCATTTCAGTAGTAATAAGCTGCAAATCCATCATAAAGTCCTCATGTTAT
GTCAGTAAGTCTACTGTAACACAACATGAGAGACTTGTAAACAGCTTAGTATCCTTCTGG
GATAAATTTTTTATAATTTTTCAAAAAATTCTGTTCGATTTCACACATAACCTTCTCTTG
ACTATCATACCCTTGGTAGAGGCTCATGATGATACCGAACAGGTGTTCCATTCCAGCACC
TTTAGCAACGCCTTGCGCTTCCATCGCATAAGTCTTTCTATCTTTACCACAATGCTTATT
GTGACAGTCAAGAACTAAAAACAGAGCTCGGTCTAAGTACTTCAGATAAGTCGTCTCAAA
TGCTTCAATTTTTCTATACGAATACTCATCATCAGCGTACATTGCTTTAAGATCGTCTGA
TGCACCATCAATAATAGTCTTAAACAGTTTTTCTGGATTATCTAATGAACTTTTTGTACT
ATGAAGAGACACATACCAGTCAGACTTAATTTTAAAATGAGAACCATCTTTCATCACAGC
AACATAGCCTTCGATGTTTTCTGCATTTTTAGCTTCTTCAACCCATTTAGGACTATCAAT
TTCGTATCGTTCAACTAAATATGGACGAAGAACAGCATCTTTATAAATATCATCATATGA
AATGTATTCGCCTGTTTCATTTTCACGAATATTCAATAAAATGATTTTCATCTCTTGATA
AGCAAGAACGATTCTATTAGTTGGAGCGACAAATTCGAAGTTAGCAGTAAATCCATCTTC
GGCTAATTCTTTAAGTCTGTCGCGCAACTGGTGATGATTAATATTCATCAAAATTCCATT
AGCCATTAAAGCCTGTTCGGATTTGATTGAACCCTTTGATTTGAACAGAATTTCATCACC
GTCTAAATAAGTTGATACCAAAGACCCATCTTCTTTTGTTAGAATGTAATCAACATCATT
TAAATCGATATTCATCGTGAATGGATTTTCATTCAAGTTAAAAAACTTTTCCATAGGACG
AGAAGCAATTCTTACCGGTTTTTCTCCATCCATTTCAAACATAATTCCACGACATTCCAA
TGCGTCTGGAAGTAACCAATCAGAATAAGATGCATAATTATATGAGAAAATTCTGTAAGT
TCTTCCAGATGCACTTACATCATCCGAGTAAAAAAACTTACGTTGTGAATCCTTACATAG
TTCCATTAAATTGTTAAAAAGTTCTTGCATTGTGTATCCTCTTTTGTGTTTTGAATATAG
TACCACACTCCATGTGGAAGCATCATTTTTTCTTATGTTGAATATTCCAAGGCGGGTTAA
ATAGCTTAATGAATAGCGGCTCCTCTAGGTCAATCGTTGCGATTGTCATTGTACCTAACT
CATTTGTCATAGAAAGATTAAAGCATTGGCGGGCGTAAAATTCAACTTTGCTTCCTTCCT
TTAGCGCAGCATGAATTAATGCAGATTTAGTAGAATCAGACGTTTTGTCTTTACGGTTAA
TAGCAGTTCTATAATAGTTTATTCTTTTACGTAAATTTTTAGTTTTTCCAATATAAACAA
GCTCATCATTTATAGCAATAGCATAAATTACGTTATACTTGTTTGGAATAGATAATTGTT
TTATACTTCCATTGTCGTCTAATTCTAACTCAGTATATTTAATAAATGAATATTCTGTTG
CAATTTCTTTCATAATAAAATGGGCCTTGCGGCCCACTCCTTAAAAGTATTTTTTAAAAC
TCATCATAACTTTATCATCAACATCATTATCAATCTGTGCAACAAGGTAAGATGACAGTT
CTACTTCTTGCGGCGCGGATTGAACATTATCAGAATTAAGGTATTCACGAATCCAAGGAT
ATGGATGTTTAACCGGAGCATCGGTAATTGGGCATGGAAGACCACACTGTTTCATACGAG
ATACAGTTAAGTAATCAATAAAGCTCCACATACTATTTGTATTTAATCCAGGAACATCAC
CATCTTTAAATAAATGAACTGCCCAGTCTTTTTCTTGACGGTTAACTTCCATGAAAATAT
CAACTGCTTCTTGTTCACACTCTTGGGCGATTTTAACCCATTCATCACCATCAGTGCCAA
GTTGAAGTTGACGAATAATGTACTGAGTACCTTTAAGGTGAAGCTGCTCGTCACGAGCAA
TGAACTTCATAATCTTGGCATTACCTTCCATGATTTCCATGTTCTTATGGAAGTTAAAGG
TACATGCGAAAGATACATAAAAACGAATAGCTTCCAATGCGTTGATTACATGCAGACAAA
GGTAAAGAGAACGCATTAGTTCGTATTTGCATTCTTCTACATGCATAAGGCTATTTGAAT
ATGAGTCCAAATCATATTCATTCTCTGAAAAACACCTAAAAATTTCTTTTGCGTTTTCCC
ATTCACGAGTTTTCTCAATAACATCATCATAATAACGACCAATAGATTCAGCGCGTTTCA
TGATAGCTTTATCTAATACAATCTCATCAAATACCTTTGACGGATCAGTATAAAGATTTC
GCATGATATGAGTATATGAACGACTGTGAATAGTTTCACTAAAAGTCCATGTAGCAACCC
ATGTATCAAGGCTTGGATCTGAAATTAATGACATAAGTACAGCAGACGGTGCACGACCCT
GAATGCTATCTAAAAGTGATTGATACTTCAGGTTATTAGTAAAAATATCTTGCTGATACT
GAGGAAGCTTGTTAAATTGTGCAGCATCCATCATCAAGTTTACTTCTTCAGGACGCCAGA
AAAATGACAATTGCTTTTCGGTTAAATCTTCAAAAACTTTATGACGTTGAATATCATAAC
GCGCAATACCAAGACCTGAACCGAAGAACATAGGTTCCTTTAAAACATCAACTGGATTTG
TATTAAAAACTGTGCTCATAAATTTTCCGCTTAGTTAATAGTTGGTGACTCGTCCGTGAG
TCAAATTATATCATAATTTACAGGATGAACAATCTTCAGCTTTTGGAGTTTCTATTTCAT
AATCATCAGTACCGGAACCATCACGGGTATTATGATAATAGAAATTTTTTCCGCCAAAAT
ACCAGAAATACAAAAGGTCATCAATCATTATTGACATTGGAACCTTTCCTTTTGGAAAAA
TCTGCGGGTCATAGTATGTATTCGCTGAAGCTGATTGACATACCCATTTCAGCATAATAG
CTACCTGCGTAAGATAAGGTTTATTACCTTTCTTAGCTAATTTCCATGTATAATCATAGA
GGTCTATGTTATGCTCAATATTGGGCACGACTTGATTAAAGGAGCCCTCTTTTGACTCTT
TAACAGAGACTGGTCCACGTGGAGGCTCGATACCGTTTGTACTGTTAGAAACTTGGGAAG
ATGACTCACATGGCATAAGTGCTGATAATGTGCTATTACGGATGCCAAAGAGCTTAAGGT
CTTCCCGCAGCGACGACCAGTCACAAACGTATTTTGGAGCTGCGATTTGGTCAATCTTTT
TATTGTACCAGTCGATAGGTAATTCGCCTCGAGACCAACGAGTGTCTGAATAATATTCGC
AAGGTCCTTTTTCTTTGGCGAGCTTAATGGATGCTTTAATGAGTCCATACTGTAATCTCT
CAAATAGTTCATGTGTTAAATCGTTGGCATCTTCATAAGAAGCAAAGTTACTTGCCAGCC
AAGCTGCATAGTTGGTAACACCTACACCGAGGTTACGACGCTTTTTAGCTTTTTCTGCTT
CAGGAACTGGATATCCTTGGTAATCCAAAAGATTATCAAGAGCACGAACTTGAACTTCTG
CCAATTCATTAATTTTATCTTGGTCTTGCCAGTCAAAATTATCTAGTACGAATGCAGAGA
GAGTACACAATCCAATTTCAGCATCAGGACCATTCACATCATTTGTTGGAATAGCAATTT
CACAACACAAGTTACTCTGGCGAATAGGTGCCTTTTCACGAATAAACGGAGTATAGTTAT
TCGTATTATCAATGAACTGCACATAAATCCTTGCTGTTCCTGAACGTTCAGTCATGAGCA
ATTCAAATAGTTCACGAGCTTTAATACGCTTTTTACGAATATTAGGGTCTTTTTCTGCTG
CTTCGTATAATTCACGGAAACGGTCTTGGTCTTTAAAATAAGAATAATAAAGTTCTCCAC
CCATTTCATGCGGACTGAACAAAGTAATGTAATCGTTCTTTCCAAAACGTTCCATCATCA
AATCATTCAGTTGAACACCATAATCCATATGACGGATACGGTTTTCTTCTACGCCTTTGT
TATTTTTCAAAACGAGAAGATTTTCAACTTCCAAATGCCAAATAGGATAATAAGCAGTAG
CAGCGCCGCCACGAATTCCGCCTTGTGAACATGATTTAACAGCAGTCTGAAAATGTTTCC
AAAAAGGAATAACACCGGTATGGCGTACTTCACCCATGCCAATCTTAGAACCTTCGGCAC
GAATCATACCAACGTTAATACCAATTCCAGCGCGTTTAGAGATATATTCAACAATTGAAG
CGGAAGCCTTATTGATAGACTTTAATGAATCACCTGCCTCAATAACAACGCATGAACTAA
ACTGCCGAGTTGGAGTACGGCAACCAGCCATAATAGGAGTTGGCAATGAAATCTGTCGAG
TAGATACTGCTTCATAAAAACGGATAACATGTTTTAGTCTATCAACAGGTTCATCTTGAT
GCAATGCCATTCCAATAGTCATAAATGCAAACTGTGGAGTTTCATAAATTTGACCGGTGG
TTTTATCTTTAACTAGATATTTTTCTTTTAATTGCATCGCCCCGGAATAAGTAAATTCCA
TATCCCGCTCGTGCTTAATTTTTGATTCTAAAAATGTAATTTCTTCTGCAGAATATTTTG
ACAACAATTCAGGGTCGTATTTACCTTCATTTACACAGTAAGAAATATGGTCAATAAATG
AACGTGGTTCATATTGCCCGTAAACATGCTTGCGAAGAGCGAACATTAAACAACGTGCAG
CTACATATTGATAATCAGGTTCTTCAACCGAAATAGAATTCGCAGCGGCCTTAATGACAA
TAGTCTGAATATCATCAGTTGTCATTCCATCACGGAGATAAGATTTAATATTTTCATATA
ATTCATAAGGATCGACTGATGTTCCTTCAGCTGCCCAAGATAAAACTTTAATAATTTTTT
GTGGGTCAAAGCTCTGAGAAACACCACTACTTTTGATAACATTAATTAATTGCATAAGTC
CTCAACTTGAAAATCGTCTTTAAACAATCGGTTAACTATATGAGCTATTATATCACCATG
ACACGGCTTTGGTTTACATGTGCATCCTAGCCTCATTCCACGTAAAGGCTCTAAATGTGC
TTTAGTTATTTCTCCGGATTTAATTCGACGTATAAAATCTTTTTTGAATAATTCAATGGC
AGCCTCCCGGCTGCCAGCATCTTTACCGACGTAATTTCCCCAAAATGTACCACGGTGAAT
ATTAACATCAAAGTCGGATTTATATTTATTCACTACTCGGCATATACGGCCTGCGCGGTT
GTAAGTGTTCATATTGTTCTTCCGTTAAAACAGTAATATCGTAGTAACAGTCAGAAGAAG
TTTTAACTGTGGAAATTTTATTATCAAAATACTCACGAGTCATTTTATGAGTATAATGTT
TTTTACCATAAATGATAATAGGCTGATTTGGTCCTGGAACTTCTAGCTCACTTGGATTAG
GAAGTGTAAAAAGAACTACACCAGAAGTATCTTTAAATCGTAAAATCATATATCCTCGCA
ATTAAATTAAAATTATACCGCCATTTTTCCTTTAATTGGCAGATGAGATACATAGTTGTT
AAGAACAAAATTATGAGGCTTTGCAAATTGACTCAACCAAAATACCTGCGATTCTGTGCC
CCATTCATCAAATTCATCTGGAAATTTTAATTGTAATTCGCATAAATCCATAGGTTCACG
ACGTAAAATTTCTTTACATTGTTCTACGTGATTCATATAGATATGAGTATTACCACCAGA
AAATATCAAATCTCCAGGAATAAGATTACACATTTTAGCTACAATATGAACTAACGTAGC
ATATGATGCAATATTAAATGGAAGACCGAGGAAAACATCTACCGAACGTTGATACCACTG
GAGGTCAAGGTATCCATTACGCACATTAAACTGATAGAACATATGACAAGGCGGTAATGC
CATATATTTAAGTTCAGCTGGATTCCACGCAGAAACAATTTGACGCCTATCATTTGGCAG
TTTTTTAATACGATCAACAACTTCTATAATTTGGTCTACACCACCGAAATCACGCCATTG
TTTACCGTATATAGGACCAAGTTCACCACTATGATATCCTAAATCTTTTGCTTGATTTTC
GTAATTTTCATCCCAGACTGTTTTGCCTTGAATTAATGAATCGTGCTGAATTAATCGTAA
ATCATTGACATTTGTGCTTCCTGATAAAAACCAAAGCAGCTCAGCAATGCAAGCTTTCCA
GGCGAGCTTCTTAGTTGTTACCGCAGGAAAACCTTTAGTTAAATCCCAGCGTAATTTAGT
ACCGAACAAAGCAATTGTTCCTGTGCCTGTTCGGTCATCGGTTTCATAGCCATTTTCAAA
AATGTCTTTAATTAAATCTTGGTATTGTTTCATTTAGTACCTTATACAAATTATAATACA
TTTTATGAAGCTAAATAATCAATACGGTTTAGCATCAAATTTTCTCTCAATTTCAGGTGT
GAAATATAATTCTTTGCCACGTTTGGAAAAGCGACCAATAATATGATAATTATTCTCATC
AAACGTGATATTACCAATATCCATAGCTTTCAACCAAATATCGATATTTCTATCCAGCTT
TATGTCAAAATTCCAATACGGAGAACTATTACCGAAAATACTGCAAGCGGATTCAGTGAA
ACAACTGCTATAAATCTTGTCTTTAGGAACTACTAATTGATAGACACCTTTAATAAACTG
AGGACCCTGCCACTTTGGCTGTTTCTCATCTTTTTCTAATAGAATAGGGTAATAATTAAA
ATTCATATTTTCCACCCCGCGGTATTTCATTTTTATATACTGATTCCGTAAGAGTTGTTA
CTTCATCTATTTTATACCAATGCGTTTCAACCATTTCACGCTTGCTTATATCATCAAGAA
AACTTGCATCTAATTGAACCGTTGAATTAACACGATGCCTTTTAACGATGCGAGAAACAA
CTACTTCATCTGCATAAGGTAATGCAGCATATAACAGAGCAGGCCCGCCAATTACACTTA
CTTTGGAATTCTGATCAAGCATAGTATCAAATGGTGCATTAGGGCTTGACACTTGAATTT
CGCCGCCAGAAATGTAAGTTATGTACTGTTCCCATGTAATATAGAAATGTGCTAAATCGC
CGTCTTTAGTTACAGGATAATCACGCGCAAGGTCACACACCACAATATGGCTACGACCAG
GAAGTAATGTAGATAATGACTGGAACGTTTTAGCACCCATAATCATAATTGTACCTTCGG
TACGAGCTTTAAAATTCTGGAGGTCCTTTTTAACTCGTCCCCATGGTAAACCATCACCTA
AACCGAATGCTAATTCATTAAAACCTTCGACTGTTTTAGTTGGAGAATAAGCGAATACCA
ATTTAATCATTACGCAAAGCCCCTTTCAAGAAACCATTCAGTGGCTTTATTAGCATCAAA
GAATAATTCTTCATATGTCTTTTCTTTGTTTTCAAAGACTGTCACACAAACACGTTGACA
TTCTCCGCAGTATTCTTCTGACATACTCAAAGCGTCAGAAAACATTTCATTAAATTCACT
TAAATCAGGATTATGCAATGCGTTAAAAATTGCATAATCGAATTCGTCATTCATAAATTC
AAATACAAAAATCATATTATTTTCCTTAAACAGACTTTTTCACATTTTTCCAATCAGCTT
TAAACTGCTCGACGTCAGAATGGTAAATCCAAAATCCTGCGCTTTCTCCATCTTCATAAA
GAGGACATCCATCGCATTCATCTTCCCATCCCATATCACGTAAAAGATGCTCAGCTTTTT
CAACAAGTTCAGAATCTTTACCGATGATATTAAAATACCATTTACCTTTAACTTGTGAAT
CTTTGATGCTCTGGCGTTGTAATCTCATTTTATTCTCCTTAGTTGATAGGGTAATAGTAT
CACACTACTACCCTGATGTAAACTACTTTTTGAAAGTTTTTCGCAAAAGTTCAATAATTT
CATCTACATTGTTTTCGTCAACAATGCAGTGAATTTTCGTTACGCCGGAAACCTTGTCTT
CGACTTCATCTTCTTCAGAAGTAGGTTCTTTATACTCACGAAAACAATGAAATTCGTCTT
CACAAACGTTAAAGTAAAAATGCTTTCCATTTGCGCATTCAATGTGTTTTATTACTCTAA
ATCCATCAGCAGAGAAAGCTTCTTTAACTTCAAACCATCCGCCATTTTCTTGAATAATTT
TAACTATTGCTGAATTAGAACGTGGGCTATAATTAATAAATGTATCAATAAGTCTTGGAA
CAAGTTCATACTTTTTACCAGTATACATAATGTTTCCTCATTTTAATAGGGGCTTGATAG
CCCCTTAATAATTATTGTTCGATCAGTCCCATGTAAAATTCTGCGTCTTCAGAATCCATG
CCATCACAGTATTCATTAGCCATAAAGCGGGTGAGGTCTTCAAGAGGACCTTCAATAACG
ATTTGAATACTCCAAAACTTAGAATCTTGTACATTTGTGATTCTAAGTTCAGGATAACGA
TTACGAATAATTTCTTCAATATATTCAAAATCAACGATGTCAATATCAACTTTAGCCATG
TTATTTTCCTCTTTAATCATCAGCAGTATTGCCGATAGTTGTATAGTACCATAGAAGAGC
AAGGATGTAAACCATTTTGTGAAAAAAATTTGAAATAAAAAAGGGGACCTCTAGGGTCCC
CAATTAATTAGTAATATAATCTATTAAAGGTCATTCAAAAGGTCATCCAGGTCCGTGTCA
TCAGCACTAGATGAACTGCCAGAGCTTGAGCTCATAAAATCATCTTCAGTTTTTGTTTTG
AAGTCATCAACATTGAATGCATCCAAATCATCAGCAACTTTATCAGCTTTCTTAGCAGCA
GTTGCAGCGGCACCGCCCATCACAGCAGTTCCCATGACTTGACCGAATTTAGTGCTCAGT
TCTTCAAACGATTTGAATTTATCTTTAGAAGTCATTTCAGAAAGGTCAACCATTTGTTCG
AACAGTTCTTTCTGGAAAGATTCATCATCAATGTTTGGAATCGCAGATTGATTCAGGAAT
TTAGATTCATCGTAGTTACTAAATCCAGAAACTTGTTTAACTTTCAGTACAAAGTTAGCA
CCTTCCCACGGACAAGTTACATCAACCGGAGTTTCACCCATTTCAACATCAACCGCAATC
ATTGCATTGATTTTATCCCAGATTTTCTTACCGAAACGGTATTTAAATACTTTACCTTCG
TTTTCTGGAGCAGCTGGGTCTTTTACAACAAGAATGTTAGCCCAGTAAGAAGTTTTACGT
TTAACAAGACTGTACTCTTTATTGTCAGTGTTGTACAGATCATTTTTACTGATGTACTGA
CATACTGGGCAAGAATCGTAATCACCGTGGGTAGATGAGCAATTTTCGATATACCATTTA
CCGTTTTTCTTGAAACCGTGATTTACAAGAATTGCGAATGGTGCTTGTTCATCATTTTTA
GACGGAAGAAAACGAATTACTGCTTGACCGTTACCCGCATTATCGAGTTTCAGTTTCCAC
TCGCCTTTATCTTCAGAAGAAAAACCACCTTTATTTCCAGCCAGTTTAGCCATTTGTGCA
GCGAGTTCAGCAGTAGATTTACGTTTAAACATTTTTATTTCCTTTTTAATTTAATTAACA
GTTGGTGCTATGACGATGTATGACCTCATAGCTGGTCAGTGAGATAATTATAATCTATTT
ATAATAAGCAATTAATACTTACAAGATTTCACAGTTTCAATGAAAACATTTTTAGCTTTC
TGTGAATCAATATTTAAAATTTTTCTATAAGCCTTTAACTTTATAGAATAATTATTCCAG
ACTAAATTATCAGTCTGTTCATCATGTTTATCAATTATATTTAAAAATGAATCAAGCAAG
ATAAACGTCTCAAACGAAATTATGTTCGATTGCAGAAGTTTAAAAATATAACTTGACTGA
ACTTTTGGATTATACTCAAAGATTTCTTTAAAAGCAGAAACTTCAACTTTTTTACTAAAA
TAATAAATGTTGCGAATATCTTCTTCAAACTTAAATTTAATTTGCTTTAAGCGTCCGATA
TATTCACGATAAAACACAAGTGCATCAGCGTCAGAGATGTCACCAATCCAAGCATCCTGG
TTAGCAACCAGATTGCTTATAAAGATTAAAGCAAGTTCCTTTAATTTATATTTTTCTGAT
AACTTCTGGAAAAAATACTTATCCCTTCGCTTTTGATAAGCGGCATCAGACACCCGCATG
CACCAATTATACTTAATTACATCATACTTTCCATTCATATGTTGTTTTATCATTAAGTAT
AATTTATAAACTGATTTACCATCAATATATCTTTCACCACCAGCAGGCATGCGGAGTTTA
ATCATAGTAGAAAATCTAATGTATTAGTTTTTTCACAACGAACAACAGAAGGACGTAAAA
GATTTTCGTCAATAGCTTCTGACTGAATTTTTTCAATTATACCCGAAGGAATAAATTTAG
CAAATTGAGTTTCAGGAATAGAGTTTTCTTCCAAGAATGCTGTCGTAGCTTCAAGATAAC
TCATTCCAAATTCTTCTACCATCTTTTCAATAATAAATCCATTTTCTTGTCGATCAAGAA
GCTTTGCGATTTCATCCTTTTCTTTTTTGATTGAAAGTTCTTTTTCTGAAAGACCGGTCT
CATCGACCGGACGAATATCATTTAGAGAAAACTGTGTCATAAAGTTCAACTACCTCTTCA
GTTTCAGCTTCAAACACATCACGGTTATCTTTATGATACAAAGCTAATAGACGATTAAAC
ATCTTACCATCAACGCCAAGTTCATCTTTGGCGCGAATTCGAATATCTTTAATCAATTCA
TTATAACCAGAAATTTTCAGTTTATGATCAGATGCTTCTTTGATAAATTTAGCCAAGTCT
TCGCCGTGGATAGCTTCATCAAATTCAACCATTTCTTTTTTAGCCATTATTCACCTCAAA
ATTCATTAATGCTATTAGTTAATTTAGAAAGACCCGCTTTTACAAAATATGAATAAATTT
TGCCACGCGGTGGTAATTTATATGAATTATAGTAATTCACAATGTTTGAAGCAATATTAT
CAGGAATATAATCAAAATCAATTAGAACTAAATTTTCTTTGTATCGATTATATTCAGATT
CGGTAAGAAGCACCTTAGCTTGCTCACGGTCATTAGCAATAGCTTCAACGATTGAAGTTT
TCATTGAAGGAGTTCGTTCACCTTCAACTCTGGTAAACCAAAAGTCAGACCGTACTTTAA
CTGAAGCAACGTTATCCTTTTTGTCGCCTTTAAGGATTTTAGTCATACAGTCAATTTCAG
CAGAACCACTTTTAATTTTAACCCATTTCTTGTGCATCGGTGACCATTGCTTAACATTTG
GATATTTGTGAAGTTGAGTAAAGTCACCATCCGATGAAATGATTAAAATCTTATGTCCTT
CTAAAGAGAACTTTTTAACAAGAACAGCAATGTGGTCATCCGCTTCATACTTATCAATAT
CCATAACAATATATGGCATATAAGCTTTCAATTCATCTATAACTTTATGGCTGGATTCAA
AATAACCTTCCCAGTCCCAAGTAGATTCTTCTCGTGCTTTTCCACGGTTTTTCTTATAAT
AATAAGCGAAATCACGACGCCAATATCCAGATTTCGCGTTATCAATACACAGCACAATTT
TAGTGTATCCAAGCGTTTTTGCTTTTTTGACATTAAACTTAATTGAGTTCAATATCAAAT
GACGAACCATTGATAAGTTAATTTTTTCTTTATCTGGGAAGTTTACTAAAGCAGTTGAAA
GCGCAATCTGACTAAAGTCAATTAAGCAAATTCCTTCTTTGTAATCTTCATCCAGCATCA
TTTCTAAATCCATATGAACCTCGTTCAATTAGTGAGATTTCTATTATATACCATCCAAAT
CTTAAAGTAAACAAGTATAAATACTTATTATTGAAAACACAATAGGAGCCCGGGAGAATG
GCCGAGATTAAAAGAAAGTTCAGAGCAGAAGATGGTCTGGACGCAGGTGGCGATAAAATA
ATCAACGTAGCTTTAGCTGATCGTGCCGTAGGAACTGACGGTGTTAACGTTGATTACTTA
ATTCAAGAAAACACAGTTCAACAATATGATCCAACTCGTGGATATTTAAAAGATTTTGTA
ATCATTTATGATAACCGCTTTTGGGCTGCTATAAATGATATTCCAAAACCAGCAGGAGCT
TTTAATAGCGTACGATGGAGAGCGTTACGTACTGATGCAAACTGGACAACTGTTTCATCA
GGACCTTATCAATTAAAGTCAGGCGAATCAATTTCAGTTGATACTGCAGCCGGTAATGAC
ATTACATTTACTTTACCATCTTCCCCAATTGATGGAGATACTATCGTTCTCCAAGACATT
GGAGGAAAACCTGGAGTTAACCAAGTTTTAATTACAGCTCCGGTACAAAGCATTGTAAAT
TTTAGAGGTCAGCAAGTACGATCAGTACTAATGACTCATCCAAAATCGCAGATAGTTTTA
ATTTTTAGTAATCGTCTGTGGCAAATGTATGTTGCTGATTATAGCAGAGAAGCTGCAATT
GTAACACCGGCAAGTCTTTATCAGGCGCAATCTAACGATTTTATCGTGCGTAGATTTACT
TCAGCTGCGCCGATTAATATTAAACTTCCAAGATTTGCTAATCATGGCGATATTATTAAT
TTCGTTGATTTAGATAAATTAAATCCTCTTTATCACACAATTGTTACTACATATGATGAA
ACAACTTCAGTACAAGAAGTTGGAACTCATTCCATTGAAGGTCGTACATCGATTGACGGT
TTCTTGATGTTTGATGATAATGAGAAATTGTGGAGATTGTTTGACGGGGATAGTAAAGCA
CGTTTACGTATTATAACAACTAATTCTAATATTCTTCCAAATGAAGAAGTTATGGTGTTC
GGTGCGAATAATGGAACAACCCAAACAATTGAACTTCAGCTTCCGACTAATATTTCTGTT
GGTGATACTGTTAAAATTTCCATGAATTACATGAGAAAAGGACAAACAGTTAAAATCAAA
GCTGCCGGTGAAGATAAAATTGCTTCATCTGTTCAATTGCTGCAATTTCCAAAACGCTCA
GAATATCCGCCTGAAGCTGAATGGGTAACAGTTCAAGAATTAGTTTTTAACGGTGAAACT
AATTATGTTCCAGTTTTACAGCTTGCTTATATCGAAGATTCTGATGGAAAATACTGGGTT
GTACAGCAAAACGTTCCAACTGTGGAAAGAGTAGACTCTTTAAATAATTCTACTAGAGCA
AGATTAGGCGTAATTGCTTTAGCTACACAAGCTCAAGCTAATGCCGATTTAGAAAATTCT
CCACAAAAAGAATTGGCAATTACTCCAGAAACGTTAGCTAATCGTACTGCTACAGAAACT
CGTAGAGGTATTGCAAGAATAGCAACAACTGCGCAAGTTAACCAGGATACTACATTCTCT
TTTGCTGATGATATTATCATCACTCCTAAAAAGCTGAATGAAAGAACAGCTACTGAAACT
CGTAGAGGTGTTGCTGAAATTGCTACACAGCAAGAAACTAATGCAGGAACCGATGATACC
ACAATCATTACTCCTAAAAAGCTTCAAGCTCGTCAAGGTTCTGAATCACTATCTGGTATT
GTAACTTTTGTATCTACTGCAGGTGCTACTCCAGCTTCTAGCCGCGAATTAAATGGTACC
AACGTTTATAATAAAAACACTAATAATTTAGTTGTTTCACCTAAAGCTTTGGATCAGTAT
AAAGCTACTCCAACGCAACAAGGTGCAGTAATTTTAGCAGTTGAAAGTGAAGTAATTGCT
GGACAAAGTCAGGAAGGATGGGCGAATGCTGTTGTAACGCCAGAAACGTTACATAAAAAG
ACATCAACTGATGGAAGAATTGGTTTAATTGAAATTGCTACGCAAAGTGAAGTTAATACA
GGAACTGATTATACTCGTGCAGTCACTCCTAAAACTTTAAATGACCGTAGAGCAGCTGAA
AGTTTAAGTGGTATAGCTGAAATTGCTACACAAGTTGAATTCGACGCAGGCGTCGACGAT
ACTCGTATCTCTACACCATTAAAAATTAAAACCAGATTTAATAGTACTGATCGTACTTCT
GTTGTTGCTCTATCTGGATTAGTTGAATCAGGAACTCTCTGGGACCATTATACCCTTAAT
ATTCTTGAAGCAAATGAGACACAGCGTGGTACACTTCGTGTAGCTACACAAGTTGAAGCT
GCTGCAGGAAAATTAGATAATGTTTTAATAACTCCTAAAAAGCTTTTAGGTACTAAATCT
ACCGAATCGCAAGAAGGTGTTATTAAAGTTGCAACTCAGTCTGAAACTGTGACTGGAACA
TCAGCAAATACTGCTGTATCTCCAAAAAATTTAAAATGGATTGTGCAGAGTGAACCTACC
TGGGCAGCTACTACTGCGGTAAGAGGTTTTGTTAAAACTTCATCTGGTTCAATTACATTC
GTTGGTAATGATACAGTCGGTTCTACCCAAGATTTAGAACTGTATGAGAAAAATAGCTAT
GCGGTATCACCGTATGAATTAAACCGTGTATTAGCAAATTATTTGCCGTTAAAAGCAAAA
GCTGTAGATAGTAATTTATTGGATGGCCTAGATTCATCTCAGTTCATTCGTAGGGATATT
GCACAGACAGTTAATGGTTCACTAACCTTAACAAAACAAACGAATCTGAGTGCCCCTCTT
GTATCATCTAGTACTGCTACGTTTGGTGGTTCAGTTTCGGCAAATAGTACACTAACCATT
TCTAATACTGATGGAACAGCAACTCGTCTGATTTTTGAGAAAGGACCTCAAACTGGAACG
AACCCGGCTCAAACGATGACAGTCAGCGTGTGGGGGAATCAATTTAGTGGGGAATCAAAC
ACAACACGTTCTACCGTATTTGAAGTTAGTGATGAAACATCTTATCACTTTTATTCTCAA
CGCGATAAAGATGGTAATATAGCATTTAGCATTAATGGTACTGTAATGCCAATAAATGTG
AATGCTTTAGGTTCTTTGAACGCGAACGGCGTTGCAACATTCGGTAGTTCAGTTACTGCT
AATGGCGAATTTATCAGTAAATCAGCAAATGCTTTCCGTGCTATTAACGGAAATTATGGT
TTCATTGTTCGCAATGATGGATCAGTAACGAATTTTATGCTTACTGCATCGGGTGATCAG
ACTGGTGGATTTAATGGATTGCGTCCTTTAGCTATTAATAATCAATCTGGTCAGGTTACA
ATTGGTGAAAGCTTAATCATTGCCAAAGGTGCTACTATAACTTCAGGTGGTTTAACTGTT
AACTCGAGAATTCGTTCTCAGGGTACTAAAACCTCTGATTTATATACCCGTGCGCCAACA
TCTGATACTGTAGGATTCTGGTCAATCGATATTAATGATTCAGCCACTTATAACCAGTTC
CCGGGTTATTTTGAAATGGTTGAAAAAACTAATGAAGTGACTGGACTTCCATACTTAGAA
CGTGGCGAAGAGGTTAAATCTCCTGGTACATTGACTCAGTTTGGTAACACACTTGATTCG
CTTTACCAAGATTGGATTACTTATCCAACGACGCCAGAAGCGCGTACCACTCGCTGGACA
CGTACATGGCAGAAAACCAAAAACTCTTGGTCAAGTTTTGTTCAGGTATTTGATGGAGGT
AACCCTCCTCAGCCATCTGATATTGGTGCTTTACCATCTGATAATGCTATAATGGGTAAT
CTTACTATTCGTGATTTCTTACGAATTGGTAATGTTCGCATTGTTCCTGACCCAGTGAAT
AAAACTGTTAAATTTGAATGGGTTGAATAAGAGGTATTATGGAAAAATTTATGGCAGAGT
TTGGACAAGGATATGTCCAAACGCCATTTTTATCGGAAAGTAATTCAGTAAGATATAAAA
TAAGTATAGCGGGTTCTTGCCCGCTTTCTACAGCAGGACCATATGTCAAATTTCAGGATA
ATCCTATCGGAAGTCAAACATTTAGCGCAGGACTTCATTTAAGAGTTTTTGACCCTTCCA
CCGGAGCATTAGTTGATAGTAAGTCATATGCTTTTTCGACTTCAAATGATACTACATCAG
CGGCTTTTGTTAGTTTCATGAATTCTTTGACGAATAATAGACTTGTTGCTATATTAACTA
GCGGAAAGGTTAATTTTCCTCCTGAAGTAGCATCTTGGTTAAGAACCGCCGGAACGTCTG
CTTTTCCATCTGACTCTGTATTGTCAAGATTTGATGTATCATATGCTGCCTTTTATACTT
CTTCTAAAAGAGCTATTGCATTAGAACATGTTAAACTAAGTAATAGAAAAAGCACAGATG
ACTATCAAACTATTTTAGATGTTGTATTTGATAGTTTAGAAGACGTTGGAGCTACAGGAT
TTCCTAAAAGAACATATGAAAGCGTTGAGCAATTTATGTCAGCGGTTGGTGGAACTAATA
ACGAAATTGCACGTTTACCGACTTCATCCGCGATAAGTAAATTATCTGATTATAATTTAA
TTCCTGGCGATGTTCTTTATCTTAAAGCGCAGCTATATGCTGATGCCGATTTACTTGCGC
TTGGAACTACAAATATATCTATCCGTTTTTATGATGAATCGAATGGATATATTTCTTCAA
CACATGCTGAGTTTACTGGACAAGCTGAGTCATGGGAATTAAAGGAAGATTATGTAGTTG
TTCCTGAAAATGCAGTAGGATTTACGATATATGCACAAAGAACTGCCCAAGCAGGTCAAG
GTGGCATGAGGAATTTAAGTTTTTCTGAAGTATCAAGAAATGGCGGAATTTCAAAACCTG
CTGAATTTGGCGTCAATGGTATTCGCGTTAATTATATCTGCGAATCTGCTTCACCTCCAG
ATATAATGGTACTTCCTACACAAGCATCGTCTAAAACTGGTAAAGTGTTTGGGCAAGAAT
TTAGAGAAGTTTAAATTGAGGGAGCCTTCGGGTTCCCTTTTTCTTTATAAATACTATTAA
AATAAAGGGGCATACAATGGCTGATTTAAAAGTAGGTTCAACAACTGGAGGCTCTGTCAT
TTGGCATCAAGGAAATTTTCCATTGAATCCAGCCGGTGACGATGTACTCTATAAATCATT
TAAAATATATACAGAATATAATAAACCACAAGCAACTGATAACGATTTTGTTTCTAAAGC
TAATGGTGGTACGTATTTAAATAAAGTAATTTTTAATAAAGGAATGACTGTTCCCGGCGC
GGGGTCAGGTGATAATGGCATTTTTGCTGGTCCTGGTGATAGTGCCACGTATGATACTAC
AAATATGGATATTGTTTCGTGGTATGGTATTGGTTTTAAATCATCACAAGGAACAGCAGG
TAGAACTGTAGTAATTAACACACGAAATGGAGATATTAGCTCAAAGGGCGTAATTGAAGC
ATCACAAATCCGTGTAACTTCGTTATCACCAGTAAATAATTATGACCTTACGCGGAAAGA
TTATGTTGATGGAGCAATAAATATTGTTACAGCAAACGCAAACTCTAGGGTATTACGCTC
TGGAGATACTATGACAGGAAATTTAACTGCGCCAAACCTTTTTTCACAGAATCCTGCATC
TCAACCCTCACATGTTCCACGATTTGACCAAATTGTTATTAAGGATTCTGTTCAAGATTT
CGGCTATTATTAAGAGGACTTATGGCTACTTTAAAACAAATACAATTTAAAAGAAGCAAA
ACTGCAGGAGCACGTCCTGTCGCTTCAGTATTAGCCGAAGGTGAATTGGCTATAAACTTA
AAAGACCGTGTACTTTTTACTAAAGATGACCAAGGAAATATTATTGATTTAGGTTTTGCT
AAAGGCGGTAGTATTGACGGGAATGTTATTCATACAGGTAATTATAACCAAACTGGCGAT
TATACTTTAAATGGCACCTTCACTCAGACAGGTAATTTTAATTTAACTGGTATTGCTCGA
GTAACTCGCGATATTATTGCTGCTGGGCATATTACGACTGAAGGCGGAGAATTTATTACA
AAAAGTTCAGGTACAGCGCATGTTCGTTTTCATGATTCCGCCGACCGTGAACGCGGTATT
ATTTTTTCTCCTGCTAATGACGGTTTAACTACACAAGTAGTTAACATCAGAGTTCAAGAT
TACAAAGCTAGTTCAGAAAGCACTTTCGCTTTTAATGGAAATGGTTTGTTTTCTTCACCA
GAAGTTTTTGGGTGGAAATCTGTATCAACTCCGGTAATTTATACCAATAAAGTTATCACC
AATAAAAAAGTTAAAGGTGATTATGACATCTATTCGATGGCAGACAGTGTTCCATTGTCT
GAAAGCACTACTGCTATTAATCATCTCCGTGTTATGCGCAATGCTATAGGCGCTGGTGTA
TTCCATGAAGTTAAAGATAATGATGGAATAACTTGGTATGCCGGAGATGGCTTAGACGCT
TATCTTTGGTCGTTTACCTGGTCCGGTGGATTGAAAGCAGGCCATTCTATTTCTGTTGGT
ACTCCTGGTGGCAATAAAGGATACTCTGAATTAGGGACTGCTTCAATTTCTCTTGGCGAT
AATGATACCGGGTTAAAATGGTACCAAGATGGTTATTTCTATGTAGTAAACAATGGTACG
AGAACTTTCCTTTACAGCCCTGAATCAACTGTAAGCCTTAGAAAAATGGTCATGGGTTAT
TCTGTAAACGGCAACGATTTAACTACGCCGCCAACAGAAAACTATGCTTTGGCTACTGTC
GTGACATATCACGATAATAACGCGGTTGGAGATGGCCAGACTCTTTTAGGATATTATCAA
GGCGGTAATTATCATCACTATTTCCGCGGTAAAGGTACTACAAACATTAATACTCACGGC
GGTTTGTTAGTTACTCCAGGCAATATTGACGTTATTGGTGGTTCTGTTAATATCGATGGT
AGAAATAATGCTTCTACGCTGATGTTTAGAGGTAACACGACCGGAGCTAGTTCAGTTGAT
AATATGACAATTTCCGTGTGGGGTAACACGTTTACTAATCCTAGTGTAGGTAATCGTAAA
AATGTCATGGAAATTTCTGATGCAACTAGTTGGATGAGCTATATTCAAAGACTTACTACT
GGTGAAGTAGAAATGAACGTTAACGGTTCATTTGAATCATCTGGTGTTACTGCTGGAGAT
AGAGGAGTTCACACAACAGGCGAAATTTCATCTAGAGCAGTGAATGCTCTTCGCATTTGG
AATAATGACTATGGAGCCATTTTCCGCCGTTCAGAAAACAGTCTTCATATTATTCCTACC
GAGTTTGGACAAGGTAAAACCGGTGATATCGGTCCACTTCGCCCGTTCCGCATGGACCTT
GATACAGGTAAAGTAAGTATTCCTAATATGGATACTACTAATTTGGTTATGAACACAAAT
GGTTTTATTAAATTTGGCGGTCATGGAGCGGGTGCCGGTGGTTATGATATTCAATATGCT
CAAGCAGCTCCTGTTTTCCAGGAAATCGATGATGATGCTGTAAGCAAATATTATCCTATT
GTTAAACAGAAGTTTCTAAACGGTAAATCCGTTTGGTCTTTAGGTACTGAAATTAATTCG
GGTACATTCGTTATTCATCATCTGAAAGAAGATGGTTCACAAGGCCATACGTCTCGTTTT
AATCAAGACGGTACTGTTAACTTCCCAGATAACGTTTCTGTCGGCGGTGGTGAAGCTACT
ATTGCTCGCAATGGTAATATCTGGTCTGATATCTGGAAAACGTTTACTTCAGCAGGCGAC
GTAACTAATATTCGCGATGCAATAGCTACTCGTGTTGCTAAAGAAGGCGATACGATGACA
GGTCGTTTAACTCTTAAAACCAACTCAGATGCTGTTGTTATTGATTATCCGGCAGATGAA
GCGGGTTATATTAAGGGTAAAAAAGGTGGTGCTGATAACTGGTATGTCGGTAACGGTGGT
GCTGATAATGGTTTAGCCTTCTGGAGCTTCCAATCCCAAGGCGGTATTAATATTAACCCT
AATGGCGAGGTTATTTTAGCCCCACAAGGTGCTGCAATGTTCAATATAAACCGCGACCGT
ATTCATATGAATGGTTCCAACTGGGTTGCACATAAATCTGGTGCTTGGGGCGACCAATGG
GGCTTAGAAGCACCTTATTTCCTTGAGTTTGGTAGCGTTGGCGAAGACAGTTATTATCCT
ATCATTAAAGGCAGATCTGTAATTAGTGGTCAAGGATATACTACTTCTGTTGACCTTGGT
ATTAGACGTACACCCCAAGCATGGGGACAAGCGATAATTCGAGTTGGTAATGCCGAAGGC
GCTGACGGTCCTGTGGGTATTTTTGAATTCCATTCGTCTGGACGATTCTATAGCCCTAAT
ATGGTTCAAACTCCTGCAATCGGTGTTGGTACTGATAATGGACTTGGTGCTCCAAGTATT
GCAATCGGTGATAATGACACGGGCTTATCACATGGTGGTGACGGTCGAGTTAATATGGTT
GCTAACGGGATTCATATTGCATCTTGGTCTAACTCTTACCATATTCATGAAGGTCTTTGG
GATACCAATGGTGCTTTGTGGACTGAAACAGGAAGAGCTATTATTTCTTTTGGCCATTTA
GTCCAACAAAGCGACGCCTATTCTACATATGTTCGAGACGTTTATGTTCGTTCTGATATT
CGTGTTAAAAAAGACCTTGTTAAATTTGAAAATGCTTCCGAGAAGCTTTCTAAAATTAAC
GGTTACACTTATATGCAGAAACGTGGAACTGACGAAGAAGGTAACCAGAAATGGGAACCT
AACGCAGGTTTGATAGCTCAAGAAGTTCAAGCTATTTTACCAGAATTAGTTGAAGGCGAC
CCTGATGGTGAAGCTTTACTTCGTTTGAACTATAACGGCGTAATTGGTTTAAATACAGCT
GCAATCAATGAGCATACTGCAGAAATTGCAGAACTTAAGTCAGAGATTGAAGAGCTTAAA
AATTTAGTTAAATCATTGTTAAAATAATAAGAGGGGCTGAAGCCCCTTGGAGGTTTATAT
GGCAGTTGAAGGACCGTGGGTAGGATGGTCTGCGAGAGTACAAACTGGAGACCCTTGGAT
GGGTGGTGCCGCACAGAGATTGAGAATGGGAATTCCGTTTTGGATGAGTAATATGATAGG
TCGATCTGTTGAAGTAATTCACACACTTGGAGCAGACCATAACTTCAATGGTCAATGGTT
CCGAGATAGATGTTTTGAGGCAGGTAGTGCACCTATAGTGTTTAATATTACTGGAGATTT
AGTATCATATTCTAGAGATGTTCCTTTATTCTTCATGTACGGAGATACACCTAATGAATA
CGTTCAACTGAACATACACGGCGTAACGATGTATGGTCGTGGTGGGAATGGTTGGGCCGC
TGGTGCAATTGGGGCATCTGATGGCGGTGTATGTATCCAGAATGATATAGGTGGACGTCT
GCGAATTAATAATGGCGGAGCTATTGCCGGCGGCGGTGGTGGCGGTGGTGGTTATTCACA
AGCCAACAACTGGGCGGGAAAATATGTTTGTGGTGGCGGTGGTGGTCGTCCATTCGGCTT
AGGCGGAAATAATGGTGCTCGATGGCCTGGTGGTAATGCTAGTTTGACGTCTCCGGGTGC
TGGCGGGAATACTGGTACAGGGTATTATGCAGGTGGAGGTGGTGAAGTTGGTCAGCCAGG
ACAATATGCTAATCCAGGTGCTGGTTATTCAACACCTCCGACAAGTCCTGGCGCTGCTGT
AGCAGGATCAGCTCCAACATGGCAGAATGTTGGAGCAATATATGGTCCAAGAGTATAATT
TGAATAAATATCCTTAAAAGGAGGGTCTATGGCAGCACCTAGAATATCATTTTCGCCCTC
TGATATTCTGTTGGGTGTTCTAGATCGCTTGTTCAAAGATAACGCTACCGGGAAGGTTCT
TGCTTCCCGGGTAGCTGTCGTAATTCTTTTGTTTATAATGGCGATTGTTTGGTATAGGGG
AGATAGTTTCTTTGAGTACTATAAGCAATCAAAGTATGAAACATACCGTGAAATTATTGA
AAAGGAAAGAAATGCACGCTTTGAATCTGTCGCCCTGGAACAACTCCAGATAGTTCATAT
ATCATCTGAGGCAGACTTTAGTGCGGTGTATTCTTTCCGCCCTAAAAACTTAAACTATTT
TGTCGATATTATAGCATATGAAGGAAAATTACCTTCAACAATAAGCGAAAAATCACTTGG
AGGATATCCTGTTGATAAAACTATGGATGAATATACAGTTCATTTAAATGGACGTCATTA
TTATTCCGACTCAAAATTTGCTTTTTTACCAACTAAAAAGCCTACTCCCGAAATAAATTA
CATGTACAGTTGTCCATATTTCAATTTGGATAATATCTATGCTGGAACGATAACCATGTA
CTGGTATAGAAATGATCATATAAGTAATGACCGCCTTGAATCAATATGTTCTCAGGCGGC
CAGAATATTAGGAAGGGCTAAATAATTATTTGTTCGTATACATCTCTAGATATCGATATA
CACCCTCAAAACCCTCGTTGAATTCGTCGATGAGGGTTTTCTTATCTTCTTGGGTTAATT
CAGAAACAATTTTACGAAATGAATTCTGATTTAACTTTCTACCTTCATGCGTTACTCCAA
TCTCATTCAGAAATGCAATAAAATTAGCACGATTTTCAACAATATCTTCTCTGGAAAATT
TAATCAAAATAGACGCAACAGTAATAATTTCACGAACTGTATCAATGTTTTTATTCATTA
ACTATACCACTCAATTAGTTGACTTTATTATAATATCATCAGACGCTTGATTTGTAAACT
GGTCTGTGTTATTTTCTTCAAAAATTTTTTCTACGAATTCCTTGAACGACTTACGTTCCT
GAGCTACATTATGCTCGATTACCTTTTCAAGATTATGACTCATTCGAAATAATCTTCAAT
TTCATAATCATGGACATAAATCATTATAGTTTTTAATACATCATCAATACTTTTTCCTGG
AGCTGGAATTACGTAGAAATATCCTGCTTTTGAGAGGTCTTTATAAGTTCCAATCAAGAA
ATCATTATTCTCAAGATGAAGTTCTTCAACTAATTCATTAACAATTGAATGGTATAAATT
TGGTAGAAACTTATATAGCTTTTCTAGAATATCAATTTTGATTGTGTATTGAACCACGGA
CTGAGAATCAATAATCATAGACCTTCCCCTTATGTTTCTGTTTGCGATTAGATTCTTTAA
ACGCTTTCTTCTTATCCTTATGAACAGAAGCTTTATTAAAATTATGTTTTGCTACTAAAT
TGTTCATAGTGTTGAATTACCTCTCTTAAACATTTGCATGTGAATGAAAACTTTTTAGCT
ACACCACATTCAAATATATGTTCTCTTAAATCACGTGTATCGGTATATCCCATCTCAACG
ATAAAGTGCCGTATTAGATTTTTATCTTTATCATTGAGAGAATTAAAATAGTCAGATTTT
GAATTAATTTCCCTGGCCAAGTTGAATCACCTTCAGTTGACGTTTTAGTTCTTTTATCAT
CTCTTCGTTCATTGCAATATAAAGATCGCGTAAAGCAGGTTTTAGCATTCCATTTACTGG
AGAACTAAATGGACACACATAATCTTTTCCTACAAGCTTTTTAGTTAATTCCATATCACA
AAACTGAAATGCAGGTTCATTAGCATAAATTCCCCAATTAGTTGACATCATTTTATTGGC
ATATTCCAATGCCTGGATTTGATTAGTAATTCCGTCAATTTGAAACTTTTTAATATTCAT
TAGTAAAGGTCCTCAGAGTAAAGTTCTTTTTCACTACCACCACGTTCAATACGTACTTGT
CCAGCGTAAGTTGCAATAATCATTGCTTCTTCACGTGTCCAGTAATTACTATATTGGTCA
ATAAACCCTTGGTCATCATCGCAAACTTGCTGAGTAACTAATTGAGGTTTAACTACATCT
AAAACTTCTGCCATATCTTTAGAATAATGACGAGCACCTGGAATAATAAGAATTCGTCCA
TCTTTTAATTTAAAACGGTTGGCTGCACAGACAATTCGTCGTTGATATTTTTGGTTTTCA
TCCCAATGTGCAGTCTGCCAACAGATTTCAGGAACTTCTTTTAGAATATCTTCTTCTGTG
CATTTATAACCATGTGCTTTAAATTTTTCAACAAGACTTTCTGGAGTCTCACGAGATAAA
GGAACATTCAGCATTTTTAAACGATTGATAAATGGGTTCATTTAAACCATCCTTTAATAC
GCTGCCACAAAGTTTTCTGTTGAGCTTTGTTAACACCAATTGAACGAATAACTGGTTGGG
ATTCCTGGAATTCTTTATAATCAGCAAGGTAAATTTCGTAAGCTGAATCTGTAAAGGAAC
TTATCGCTGCCATGAAATTATTGCGAATACCTACTGGAGCATCTTTACTTTCACGAATAA
TCATGTATTTACCAGTCTTAATCTTTACGATAGTTCCAAGATAAGCTCCGTGGTACCAAA
TATCCCACCCCTCTTGAGTAGGTTCTGCACAGCGACGAAGTTCATTGATAATTTCTAACT
TGTTCATTATTTATTCCTCACAGTTCAGATGCTACGGTGATTACAGCTTCAATGTTTTCT
GCAGAGCGTTTAATGTCAAGATACACATTACCATTTTTAGCGATTTTACATGACATTCCG
ATATCAGTAAATTTCTGAATATGATGTTCCATCATTTTGTATCCAAAAATTCGCATATTT
CCGTTGTTATTAATTTCAAAATTACGAATTCCGTGAGTGCGTTTTTCTAAAATAGCAAGA
TAATTACTACGATAAGTTTCAACCTTTTTAAGAACAAATCCGTTTTCATCCAAAAGTTTT
AACATGAAGTCTTTATCTTCTTCCATATCAGAAGTAATCTCACGAGCTTTACGAGTTGCT
CGTTTTTTCAGCAGTTCCGGGGCATTTTCCTGTGCGTATAAAGTTGCTGCATTTGAAATA
ATATCCTGAGCTTCACCAGTAATGATTAATCCATCACCAGATTTCTCCACCAGGCCTTTT
TTAATCAATACCCCAATATTACTATTAACTACTGCGTTACCTAAATCTGGATGCACCTCA
CGAACTTCTGCAGCTGTAATGAAATCTTTCTTAGCAATAGTAATTAAAATCGCAGCAGTT
TTTTCATTCAGAACATCGTTAGAAGCTTTGATGATGTAAGTTACTTTAGACATTTTCTAA
TCTCCGTAATTCTGTATCAGTAGTTGATAGTTGTATAATACCACAGTATGCTTTGGTTGT
AAACAGTTTTGTGAAAAAATTTTTGAAATAAAAAAGGGAGAGCCGAGGCTCTCCCTAAAA
TTACTGCATGACTGTGATAACTGTCATGATAACACGTTGAATTCCGAACGCAAGAAGACC
TCCTGCTACAGCTGGAACAATACCTAAACCCGCCAGCAAAATGCTACCGGATACTAATGC
AGCGCTTGTAATGCCTATGAATGGACTCATTTGATTTCCTCTAAATCTTTGGTGTATTCT
GTAACTACATTAGTAGTTTTCCAATATTCATTTTCTTCTTTTTTAGCTTTAGCTTCTTCA
GCAAGTTTCTTCGCTTCATCGGAAGTCATATGAAAAATATTCATTCCGACTAGTTTATCA
ACATAAGAAGAATACATCTCAATTTTAGAAAGTTCTTCAGTCAGTTCTTTACGAGTTTTA
CCCTGTACAACAATTTCACCTGAAATTACTTTCTTAATGAAATGTGCCTTGGCAAAGGCT
AAACGAAATGCTGATTCAGTTTCTTTAATTTTGTTATCAATTCGTTTTTGGACATAAGTT
TTACGAACTTCAACAAAGTCTTTAATTAAATCAACTACGTTATCGTAAACTTGTAGCTTT
CCTTTCTCATTAATAACGGTAATATTTTGGGAACGACGCTCAATCAACCCAAAGTCTTTC
ATAATTTTTGCATGGCGTTCTTCTTCATTATCGCTCAAAGAATATTCTTTACGGAATTTA
ACTTTAAAGCCAAAACCATGCTCACCACAAGCATCGTCCCATGTAATGAAGCCTTTATCT
TCAAGTGGGTCTAAGATTTTACTCACATAAGTTTCACGATCATACTTATACGGAATCTCA
GTGATGTGCATTTGAGTTCGTGAAGTAAACTTATATGTTCCACGAATTTCATATTGTCCA
TCAATTTCAACAACTTCACCACGAAATTCTGGGAATTCTACCTTCGGTTTAGTTACTTTC
TTTCCTTGAAGAGCTTGTAGTACAGCTTTCTTGACAGAAGAAACACTATGAGGAAGAATG
TAAGTTGCATAACCAGTTGCAATACCGGAAACGCCATTAAGAAGAACAGTAGGAATAATA
GGCAAATAGAAAGCAGGCGGAATGTGTTCTTTATCTTGATGTACCGGAGCATACTCAGTA
TCTTTATATACGTTATAGAAATTTTTACTTACACGAGCAAAAATATAACGACTTGCTGCT
GCCTTTTGGACAGTACGAGAACCAAAGTTTCCTTGACCATCTAATAGAGGAAAGTTATTA
TTCCAAGTATTAGCCATCAAAGCGCCCGCATCTTGTGCAGAGTTTTCGCCATGATGATAT
CCAAGGTCCGCTACACCACCTGCAATAGAAGCGAGTTTGTGAAACTTATCTTTATTTCCT
CGCGCCAAATCAAGAGCTCGAGCAATAACAAATCGTTGAACTGGCTTAAATCCATCAATC
ATATTTGGAATAGCACGATTTTCAACCGTGTACATAGCATAAGCCAATGCTTCATTATCA
ATGATACTTTTTAAATCGCGATTATTCAGTTGCATAAATTTACCATACTAGTGAATGTAG
TGCCATAATAACATCAGAAATGAAAAGCACGACTTGAATTAATCCGAACATTATTCCGTA
ATATAATGCTATCAATAAAATAGCAAGGGCTAGTGAATAGCCCAAGATTTTCTTAATCAT
TAGTAGATAACAACACAAATGTTAAACATGCACACATACCCTGAGCTAAAGCCTGCGAAA
ACACGCTACTGGAATCGGTATAGATAGTTAAAACGCATACTATTATCCAACAAATAAATG
AAATAACTCCTAATAATTTTGCGATATTCATATTTTCCTCACTGGCGTCCGAAGACGCCT
TTGGTTTTAAGATTGTTACGATAGAACTGCATCACGTGTTCGTTATGGAAATTACTCATT
AGTATGCCTGCAAAACGAATTTAAAGTTATCAGCCAACATACGGTTCATTTCTTCAAGTG
TTTGATACTCAGAATGATGATTACGAGTAAACGCTAAAGCTAACTGTCCTTTTCCAAATC
CCGTCGTCAGAGGTTTCATCTTAGAAGCAGGCAGATAAAATACTGTGTATGGAACATTGT
TATTTGCAATAGTACGTGCAAGTTGAGACCGACGCTGGCGAATATGACTTAGAACCGCAC
TGAATCCTTGCTTAGAACGCTGATTACCTACATAAAATCGTGCAGACACACATGGATTAC
TAAATGGACGACCATCTAATTTACTTACTAAAAAGTAAAATCCAGGTTTAGATAAAATAT
CTTTATGCGGAGTTCCTAAAAACCATTCACCACCCTTGATTGTACCAATAACAGTTGCGC
CTGCATTGTTCAGATCAGTAACAGTCATGTATTTCATATTAATTTCCTCTAAATTATTTT
CTACTCCAAGGCCGCATGAATACACACGGCCATTAAATTAATCGTCGCAGTCGACGCTCA
ATTCCCAAAACTCTTCTACAGTATAAGTTTCAGTATCATTTTCAATACAGAAACGTTCAT
TACTGTTATTTGCTAAAGTAGCATTAACTGTAGGTTTCTCGCTAGTGCTCTTAAGAGGTG
AAATGCGAATTAACTGATCACCGTTATCTAAACAGAAAATTTCACCAACTTTTACATCTT
TAAAAATTTTCATAATTCACCTCAGGGAGTATAAAATCCAAATGCAGTTGTTGACCATCC
CATCCAATACGGGAAATTTGCGCCAATGTAAAACATGAGAATATAAAACCAACCACTCAG
CAAATTCATCATTTTACACCATTCCAAATTGTTTCAACTACGGATTTTAAACCATTTTGA
TGAATATCAATTCCGACTACTGTCATCAAATAGATTCCAACTACAACTGAACCTAAGGCG
AAAATCAGCATGAAAATGAATAAAGCTGGAAAAATATTATCGAAAAACCATTCAATAAAT
GTAAAAGCACTGCGTTTACGCTTCATATTTTCCTCACATAAATCCAAAGTAAGCGTTTAA
TACATCAATCATTAAAACGATTGGGAATATACTCAAAACTACTAGTATTATAACTACATT
CCATATAGCTTTAACAATCTTTTTCATTTTCTGTTCCTCCGTAGTTGATAGGGTAATAGT
ACCACAGAGGAACAGTCTTGTAAACAACTTTTTTAAAAATATTCGTAATAAATGTGAATA
CCAACTACTACCGCTGAAACCTGTGCAACCCACCACGCACAAGCAATAAGTACAGAATTA
AAAATTTTCATAATAACCTCATCACAAAAGTAAATGTTAAACAAATCAATGGAATACTAA
TTAACCAAACAAAACACCACCATAATGAACTCATAGTTCAATCTCAATGATTTTCATTTT
ACTCAATAATCCGCTAAAAGATCGACGATTGCATCACGAAGACACTTATTCGCAATACGT
TTAAGAGCAATTTCAGTTTCTGGAGCATTTTGAATAATCATCGAATTCAACTCAGCCTCA
GCAAATTGAAGTTCTTTAACTAGTTCAGCTTTATATTTCCCTTGCCCTTTCTTAGCTTTT
GCCCGTTTTAATTCAGCTGTAGTAAACATTTTATTCTCCAAATCCGTATCAGTTGATAGT
TGTATAGTACCACAGTCCTTGTGGTATGTAAACCGGTTTGTGAATTTTTTTAAATGGAAA
GATACCATCCGTTGTAGTTGCTTTTTCTTACAACCTTACGAAGGTCTTCTCTATCACCGA
TGAATTTCGGAGTGTACTGGATGACACCTGGATGAATTTCTTTAGTGTTGAATATAATTA
TACAGTCCGCGACCTGGTGATTTAGAATGGGCCCTAGATTTATTCCAGAACCGTATGGAT
ACTCTCCGCTGCATCCTGTTGTTACAGAAATCCAACGTGAGTCAGTTTGATGTGTCTTAA
CTTCTACACGAAGCCCGCAGTATCTTGGATGCGCCAATACATCCCATGCGTATGTATACG
GGTCATTAACGTCCTCTTGGCCTTTGTTAACATACCCGCTTAGCCAATCTGCTACAAAAA
ACTCTGCGTACACCGCGATACGGCATCTTTCGATAACTTCTGCTTTATCCTGGTTCGGGT
TTTGTTTTAAAGAATATCTTGCTGTATCAGCAATTTTGACCTTCATTTCACTAGTCAAAT
CACTGTTCGATAGGGTAAATGTCGGAATCTGAAATAGTCTCTGTAAACCCGGATTCGTTT
TCTGCATTTAGACTTTCCTTTTTACCGCTGAGATAAGCGTTATATACTTTAAGAGTGCCG
TAATAAATTCGGTCATTTTCATCTAAAGACTCACGGTCAAGTTCATCGAGTTCCTTTTTA
TCCATGACTATAATATCATTGCAGTAAAGAAACCCAAGTTTTTCGTGACCAAACTCAAAT
TTATTACAGTACACAATATTAGCGTGATGATTTCCATGCAGTTTAATCTCTTTTATGTCG
GAATCACCGATATTCATACAATAAATCATAATTTTTCCTTAAAACAAAAGGGCCGAAGCC
CTTTATTTTACTTGAATTGTGCAATTCTTTTCTCTAGACATTCAGCATAAGATTTCATTG
AGATGAACTGTGAAAGCAGTAATTCTTGCTCAACTGCACTAACTGTTAGAAACTTCGCGC
TTTCTAAAAATTTACTCAGTGCATTAATTTTGAGCATTAATTGATCGTATTCTTCTTTTA
CTCGTGCTTGATAACCTAACATAATTTTCCTTAATATGATAAGGGCCGAAGCCCTTATTT
AAATTGTTCAGTAACATCTTCAACTACTTCGTATTGGCAGGTACGCATTTTAGCGTCGTT
ATAATCAATCGGAATTGATACTACATCGCGAGGATGTACTTTAACTTTTACAACTCGGCT
GGTTGAACTACCAAAGTGACGAATATAAGATTTAGAACACACATGCAGACCACGAGAACA
GGTTTGTGTATCATCGTCGTTCACACGAGTACGTGGCATTTTAACTACTTTACCCGGACT
GTTATCAAAGGTATTTGAGTGACAGTCAAAGTAATTACTGCGAACTACTTTCCAAGCATA
GAAGTAACCATCTTCTGTAATTTCAATATCGTTTGCTACCAAGAAATCAAAGAGTCGAGA
TACCGCTTTTTGGCTTGGGTTTTCCAGCAGATTTTCCAAGAACGGAAAATAAAATTCAAA
GTTTTCGCCTTTTTCCATCGAGTCAAGAATACGATCAACCAGACCAGACCGTAATTCAAT
ATTTTGATAGAACAAGCTTCCACCTTCAATTCGAACATCGCCGGAAATATATTTTTCAAC
AGCGCGACGAACATTAATTTTTTGTGCCGCTTCTTCCAATTTATCTGCAACAAGCAGATT
AAGAATTTCCTGGAAGTTTGAATGAGTATTAGGAGTTGCGTTATAAGTTACACCGTCAAC
AGTAATTGAAATGAATTTTTTAGAAGCATTCCAAATAATATCAGATTTAGCAACTGGAGC
AATAACTGCATCGCTATTAACTTTAATTGTAATATCACCGCTAATAGTAACTTTAGAGCG
TTTAGCTTCTTCAGCATTTTTCAAAACACGACGGATTGTGTCAACCGATACACCTTGCCA
ATCAGCCAATTCCTGTTGGGTGTAATTACCACTTGAATACAATTTAACAATTTCAGCTTG
TTCGTTTTTGGTCAGGCATTTAATATTGTACATAATTTTCCTTATTAGGCCGCAAGGGCC
TTCATAGTTTTAGCGATTTGGGAAACTTCATCATCACTTAAAGAATTGCGATAACCGATG
AAGTCGGAAACAATACGGAATTTCTTGGTAAACTCAGCAACCATTTTATCACTGTTTTTT
GAAGCATTACGTGATAATTCATCAAAGAGATTAGTTACCGTCCAGATATCATGACCAATG
GTATCTTTTCCACCATTAAAATATACACCCTGTAATGAACTAACCATATTAGCGAGTCGT
GTATATTCTTCAGAAACTTCATCTACACTGAAGTACTTCATCATAAAATCTAGCTCAGGA
TACTTGATAATTTTATCAATATATCGTTTAGCTGAACTTGAATAACCTACATACTTATCA
TAATCTACATCATCAAAAGCATCTACATATAAATCACGCAAAGCTTCAAAAATACATTGG
CACTGACCGAGTTCTTTTACCTTTTTCTGTAAAAGCGGACGAATAACATAAAATTCATTA
ATGCCAATAAGATTAGCCATACGAATCAAAATATTCATAGATGGATGACAAAGAGATGTA
GTACCATCCATAGAGAAAATATCAGAACGATGCATATACGCTACATAACCAGTAATTTCA
TCTGCTTCTGACGTAAGCGTAAATAGTTCCTCTTTTTCCCATCGCCCATCTTTAATTTCA
AACTTAAATGCTGTAGCAGCTTTAGGACGAGGAGCTTTACTTTTAACTACCTTTGGAATA
TAGCTTTTAACTAAAGCTTCAATTTCTGACAAATAATGAATGTTAACTTCATCACTTTCA
AACATCGCCATAATATCAGGGAGTAAATCAATCTGCGATTCTACTTCTGGATTAATAAAC
AGAAGACGCTCATTGTGATGAATATTCAGAGTGTTATTAAATTCACTATCATCTAACGCA
CGTGCTAATCCACGGACAATATTAACACGATTTTTAATATTATCAATAACGATATTAATT
TTTGTTGTATTAATACCAAACAGACGATAACTTGATGCAACGGCTGAAGTTTCATGGCTT
TGCTTAATACGTTTCAGCCGAGGGTCAAGATTTACTTCATACACAACCCCTGCATTACAT
AACTTACTATCAGGTTCAAACATACTCTGCATCTTCTTATATGACAGATTTTTAGTCGTG
AATTTGACCGAATTACTAATCATATAATCTCGAGCAGAATACCCCATCTTCATCAATTCA
CGATATGTGTGACGAGGAGATGTAGATTCTTTAAATCGTTTTACATCTTCATTAAATGCT
TTCTCACTGAGTTCTTTAACTCGTTCAATAATATTTTTACGAGTACGATCATCCAGTGAA
AGAGCCTCACGAGATGGAGCAATATCAAGTGAACCCATTGGAAACTTAATGTAATTCACT
TCATTGCGAATGCTTAGCCAGTTACGGTCTCTAATAACACCGTCGATAGGATAAACAATA
CCGCCATAGATAGCATATAATCCGCCGCGATCTGGCCAGTATCTTTCTGGATTTACACCG
TAATAATCATCAAAATCCGGAAAATAATCAATTTCGCGGTCAAGACCATTAATGATAGCC
AAATCTTTGAACGGTCGCATGATATAAGAAACTTCATAAGCAAAGTTTCTAAAGTCTTTT
TCTTCAACTGGAACTACGATTTCAATACCAGTTTTATCATCTGGACCCATTTCTTTTACG
AATGTAGGTTTAATCTGCGGACCGTCACCATCCATGTAAGCTACATAACCACGAATTTCA
CCTTTATGATACGAAGTAATACTAAATGTATCAGTATAACTAAACGGAGATTTAGAACCT
AAACCAAATCCGCCAATGAAATCGTTAGATTCAGCCTTAGATGAACTGAAGTATGAATTA
TACAGACCAGGAGAATTATCATCACCCTGAATATCAAAATCACTCATACCTGGACCAAAA
TCTCGACAAACAAATCGTGGATCTAAACGTCCAGGAACTTGTATGATAAATTTTTCAGGA
TTTCCATTGAGTGCATGGGCATCAATCATGTTGGTAATCAATTCACGGACTACTGCGCGA
ATTTTGTTTGTATACAGATCAGATGATAGAATTTTAAATACTTTAGGAGATGCTGTGATG
CTAAATGCTTTTGATTTAGAACCATTACCAAGAATTGTTTCTTTTTCAGTGGTGATAATC
ATAATTTCCTCATTAATTCATATTACGCTTAATAACTTCAGCAACTTCTAGTAGTTCATC
TTTAGTTGCGATGTCGGATTGAATTTTATCTCTAATATCTTTAAAGCGGTTTTTAAATTC
TTCGGCTTCTCCCATATCGAAAAAGCGTTGAATGATTCTATATTCTCGATGAACTGCTTT
ATCAAAAAGTTCTAAATTTACTTTATATGATTTCATTTCAATATCCTCATTTGCCCAATT
AATTATACCACATCCTTGTGGTAAAGTAAACTACTGGCTCATCCATTCTTTACGAAGGTC
AGCGTTATCTCCCATGAGCATTTCAAAAAGCTCTTTCCAATTCTCAGGAAGTTTAACAAC
ATCATATACTGGGTTTTGAATCATCTCACGATATTCAGATTTTTCCAAAGAGCCAAGTCC
CTTAATATAACGGATGCTATGTTTAGGTAGAGCATCTTTGGCACTCTCATATTCAGCGAC
TGTATAAAACCATTCTTGTTTTTTACCGACCTGAGCGATGATTACAGGAGTTTTGACAAA
GCGAATTCGTCCTTGCTCAAACAATTCTGGCCAATTACTAAAAAACCCGAGCAGAGAAGG
ATAAATGCTTCCTAGACCATCGTGGTCAGCATCAGTCATAATAGCAATATTATGATAATT
CAGGTTTTCAGCTTTTTCACCAAGAACTAATCCAGTGATTGCGCAAATATCAAACAGTTC
TTTGTTTTTAAGCATATCGGCATATGACATACCCCAGCTATTAAGAACTTTACCGCGCAA
TGGATAACCACCGTGAAGTTCTTTATCACGAACATCAATAAGATATCCGATAGCCGAATC
ACCCTCAGTCAAGAACAATGTAGTGTCAGCATCTTTACCGCAAAGATTCGCTTTAATATG
TTTATGAACCTTTGCCTTAGAGGCCTTTTTAGCTGCTTTAGTTTCTGCTGCTTTTTCTGC
CGCCAATTTACGAGCCAAAGCAGCTTCAATAATCGGCATTAGAATTGCTTCATTATTTAG
AATAGCGCGTGAAATCTTTTTAGCATCAAGCTGAATATGACTACGAATTTCACCAAATGG
AGAAGTCAAACGCTCTTTAGTCTGAGAGTCAAATCGCATGTTTTTCATATCACGAACAAA
CATAACGATAGTCAAACATTCTTTGACACGTGCTTTAGTTACATCAATTTTGAACTTACG
TTTGATTTGTGGAATAAGGTCTTCACAAATATCATCCATAACACAGTCAATGTGATGGCC
ACCATTCTTAGTGTGAATGTTATTCACATAAGTTAATTGACGAAAACCATCCGGTGAACG
ACCAACTGCAATAGAACAATTTTCTTGTTCTTGAACGATAGCATGCTCATCATATTGGCG
TGCATATTTCTTAAAATTACCCTGAACCTTTTTACCATTAAAGGTAAATTGAATATCAGG
ATAAACAACAGCAAGTGTCTGGAGACGATCTAGTGTAATGTCAAGATAAACTTGGGACAG
CTCATTAGTTTCAAATGACATAAAATCAGGAATGAAAGTAACACGAGTTCCTTTCCATTT
TCCTGGAATAGTTTCCCATGATTTATTTTCCATGCCATTTGAACAACGAACTACAATATT
ATTTTGACCATCGCCAGTTTCACCGACAAACATCACAGAAAAAATGTTAGTTAAACTAGA
ACCAACACCATTCATACCACCGGTGACGCGTTCTTTATCATCACCAAAGTTACCACCTGC
TTTTGGAATAGTCCATGCAGCAACAGGACCAGGAATTTCTTCACCGGTAGGTGTTTTAAC
CATCGCTTGTGGAATACCGCGACCGTTATCTTCAACTGTTACCTGATTGTTTTTAATGGT
AACATTAATTTTATTTGCGAATTTAAACTTAGTACGAATACCTTCATCTACTGAGTTATC
GATAATTTCATCAATAAGCTTAACAAGACCAGGTACATACTGAACACTTTCCCATTTACC
AAACAGAAAGCGCTCATGCATTTCATTAGCAGAAGAGCCAATATACATGCCGCTACGCTT
TTTGATATGTTCAATATCGCTCAGAATTTTAATTTCATTCTTAATCATCACTTATCCTCG
TTTGGTTTCGGGAATATTATACTCCAATAATCATAAAGCTAAAGGCCCGAAGGCCTTTTA
TTTAAAACGAATAGTTGAATCCTTAAAGAACAGCCCAGAACATACTGTTCCTTCTACTTT
CTGCCCAGTAGGTCCAATAGCACGAAATCCAGTATGCTGGAAATCATTTTCAGAGCAACC
GAACCAATTATATCCAGTGATTTCAATATTAGTAAAACCGCTTGAAGACAAAACTTTAGT
TGCATTATCAGCGTCAGTACATCCTATTAAAGACACTGCTAATACTAATGCTGCAATAGA
ACGATTAACATATTTCATAATTTTCACTTAAATTTAATGGCTTGAAGAAGACTAATAACT
CTCAAGCGACTTCTTTCATCTTTAACTATAAAAGAAAGAGGGTCACCAGATTTCATAGTA
ATAGTACATTCAAAATCAAAATCTTCGGGAACTTCTTCGAATAGGTCAAATTCTTCATCA
TAAATTAGAACATTACTCTGAAAACTGTGAAGAATTTTTCCATCATTTCCAGATGTTGTA
CTAATCATTGTAACATTATTACCTTTCATATCTTCAACGATAAATTCGCTTGTAGATATT
ACAACATTAATAGAACCATTCCTATAAATAGCAGAAAATAGATATTTCTTTTCTTCACCT
TCGCGAATGCGATATTTCTTACCGATTTTAAACATAATCACCCTTTAAGTAAGTCGTAAA
ATCCACCATTCACATGCTTAGGAGCAGAAGGCCGACGAGTAGACAAACGATGACATTTAG
GGCAAACATCATTTTCTCGTTCAGAAATCTTTTTAATTTTTTCATATTCATGCCCACAAT
CTTCGGATTGGCATTTATAATCATACATTGGCATAATTATTCCTTAAAGTGAGCTTTCAA
CATCTGATACAAGGACCATGCCTGTTCATTATTTTCAATAGTAACTTTCATGACAGGAAA
TTCTGTGAAATCTTCTATTTGTTCTTGCTCTTCCTGCTCTTGCTCTTCAGCAGCCTGATA
TGGATTTTCCACTTCATTAAAGAACTCGGCTTCGTTGCTAGAAAGCCAGATAAAGTTTTC
GTCAATGACTGTACCTCGTGATGAATATCCAACTATGCCAGTAGATGTCACAATTTTAGT
AGGACGCCCGTAATAGTCAACATCTAAAATTTTAAAAGGTTCCATACCTAAACGCCGCGC
ATAGATTCCGTTATCAGTATGGTCTTTAATAAAATTTTCTTGAGCTTGTTTATTTTTAAA
TTGATACCATTTATTAACTTCAAATTTAATAGCCATTAATAAATTTCCTTCCAGTAAGTT
GTGCCATCTTCAGTAATTTCACGAAATACACCATAAATTGGTTGTTTATCACCGACTTTC
TCATACACATAAACAGAAGTCAAGTGAGTAAACTTGCTAGTATGTTCCTTTTGAACTATT
ACCAAATTTGGATCAAATAATACATCTTCAAATTCATCATTAGTACAATTCTGAACAATT
TTACGTTTCATTACAATTTCCTCATTAATTGAACAGTGGAGCGATACGTTTCAGAAGAGT
AGCAGCACCTTTGGCGAATTTTACATCATGAAAAACAGTTTCTTCTTCCGTTACAACACG
AACTTTAATGTTTTCACAATCCCAATTTTTACGAACAATCTTCAAAAGCTGCAGTAAATC
TTTACATTCAGCAATATCGATAACACATCCACGAGAATCTTCCATTATCGCACGATAACC
GTAAGCCATACCATAATCGTTGAAAGATTGCTCAATGATAATTAAGTTTTCCATTTTATT
CTCCAAGTTGTTTTCTGTATCAGTAGTTGATATTGATATAGTACCATAATCAACTACTGA
TGTATATAGTTTTATGAAAAAATTTTAAACTTTATGCACAGCGAGCTTTGCTATAGTGTT
TAATCCAACTTTCAGGAATGACTTTGTATGTTCCTAAAAATACCGCGTTGTACAACTTAA
CGCCATCTTCTACCCATTGATCGGTAATGTATGCACACATAGCACGAGTACGATGTTTTT
GAACAACCCCTTCCTCATCTTCGGTAAATTCGAATTCATAAGGAGCAATGAACTTAATAG
CCTGACCGAGTTTCCACTTAAAGTCTACACCTACATGCGAAGTATCAATCGTTTCAATTC
CTTTAGCAGGAACAGCTTTCAAAAACGCAGACTCAAGAAATTTCGCACGAACATAGCCAA
ACTGAGGTTTAGACTTTCCATCTTTAGGAATGATACGCACTTTTACTTCAGAATCTTCAT
CTTTAACACCGTGTTTAAGCTGAATGCTTACAATTTCAACCAATTTTCCTGCTGCTTTAG
AACGGGATTTATCAGATACACGAGCAATTTCACCAATATTAATAATCATAGTTATCTCTC
ACTTGTTAAAAAGATTTTATACTCCACGGGACCATTATACTCTGGTCCCAAGAGTTTGTA
AACTATTAATTTAAAATAGCTACCACCGCACTACGAGGTACTACGCTAAAATCACCTGCA
TGAACAACGTTCAGAAGTTCAACACCATCTTCAATCCATTGGTCTGTTACCCAACCACAG
ACCGAAATAAATGGAGTCTTGATAGCTACCGCATGAGTTAGAAGGTCAGTAGGATCAACT
TCTTTGAAGAGGATAAGCTCTGCTTCAAAGACTATAGTTTTAATTACTTCATCATTATCT
AAACGGTGTACTCGAATCACGTAAGCTTTATTCTTATCATACCAGTTACTTTTTGAAAAA
CTTTTAACCACAAACTCGCCTTCACCAAAAGCATTAGTCAATGCTTTATAACCCAAATTT
AGTTCAGTAGTTTTAATTTCAGTTTCAACCAGTTTGTAGATTTTACCAATTTCGATGGTC
CGAGCAGTCATAAAGGTTTCCTTAATTTCTAATGGTTTAACAAGAGTGCCGTACATGCTT
GCTATATCCATGTCAAGCAGCTTATATACAGGATTCGGTGCGTATTCAGGATTATAATTA
AATTTCATAATTATCTCATTTCAATAAAATCTACGAGTTCAGCATGCGATTTACGGAACA
TTACTTGGTGCCCGCCAATGATAACTTCATCTTCAGGAACTTCGTATACAGCGAGATAAA
ATCCTTTAGAAGATAATTCTTCCCGCTCTTCTCGTGTGAACCATCTCATCATATCATATT
CACTAGCGAAAGCAAAATGATAAAGAGCTATAAACCATCCTGGAATATGATATTCTACTC
CAACATAATCTTTCTTGAACTTAGTATTAATTACTATATTAGCGTTTTTAACTAATAGTT
TATCTTCGTGTGGTAAAGGAATTCTTTTATTATTATCGCTATGATGCATAAAATTAGGTC
TGTCATAACCTACGTGTAATAGCCACTCTTCGCTCCATGAATCTATTATACTCCTGTACG
GCGTTATTTGAACACAAAGATTTCGACGTATTGTTATAGCATCTTCATAATCAAGAATAC
TAAACGATGATTCAACACGATAAATTTTCATTTTATTATCCTCAGTAGCTATGGTGTTAT
AATACCATAACTAACCGAGGAAGTAAACAACCTTTTATCGTTTAGTTGGAAGAGACAGAG
GATCGCAATCTTCCTCTGACGGAGCATCTTCAAGACCCATAGCATATCGCAAAGCATACT
TCATCATCAGGATGTCTTTCGCACAATCATGAATAGAATCATGTGCAACGAATCCATCTA
AAGTTCCCTTTGGAAGAGGACATGTAGTCATATCACGAACAAGCAGAAGTGCTTCAATTC
TGGTACGAATATCACGCTGATTCCAGAATTTACAAGGTTCTAACTTAAACGTATCAAGCT
CATTCTCAGAAACGCCGTTAAGACGTTGAATATCACGAATGAGATCGACTAAAATTGGAA
AATCAAATGACATTCCACGGCACCAACCATGAGATTTCCAAGGATCGATATTATGCGCAT
TAATGTAATCATTGAATTTTGCAATACCATCAATAGTGCTTACATCTTCATCTGATGGTG
CAATATTTTTTCGAGCTTCAGGGGATTGATTCTTCCACCATTCGATAGTACTTTTAGTAA
AAAGACGGTGTCCTTTTTGGCTTTTTAAATCAAATTTGATTTTAATGCCACGTGAAACTA
ATTCATCAAATGTTTCAACAACTTCCGGGTTAGGGTCAAAAGCAATTACAGCCAAATCAA
TAACAGCTGCTTTTTCACCGCTTCCCATTGTTTCAAAATCTATAATAAAATCAAACATTA
AATTTTCCTCGCTAAATCACGAATTTGACCTACAGTATAATCTTGGATATAAACTTTATT
AATGGGCTCATCAATAAATTTTGCCATAGATTCAATATCTTTTTGTATCTCTTCAAGACT
GTATACTATCTTTGAAGCTTTTTCGCGAATAGTAACATTATCAGGTCCTGGATTTTCCTC
AATGACAGCTTTAACATTTGTCATAAGAGATTTAAACTGATGCAAACTTAATTCAATCAT
TAATAATCGCCTTATAAAGATGGCTAATTTCACCTAAAACATAATCACTGATTGTAACAG
TTTTTACTTCACCACAAAAGAATTCTAACGCAATCAAATCTCGTTCAATTTCTTCCAGTT
GAAGCATTAACTTGCCTGATTCAATTTTCACGTTTTTGCGATTTTTACTATAAGCTATTT
CATAAATTTCGCTCACTTTATCTTGAAGTAGATAAAACTGGTCTTTAGTTATTTCCATGA
ATAGCTTCCTCAAATTTAATCATACATAAAACACATCATAACGACCACGAGTGACACCAA
CATAAAGAAGCTGTTGAGCCAATTCAGCGTCTGCATAATGAATACAAGGCGTATAGATGA
AAGCACGGTCCACAGACATACCCTGAGCTTTATGGAATGTTGATGCAGGAAGTGCTTTCA
CTTTACTAAACTGCGATTTAGCATCCCAAAAATCACTCCACGGAGCTTTTCCGCCTTTAT
TCCAATTTTTATAAGTTTCTGCCGTTTTACCTAAAAATAAGTTAAACTTATACAGCTCTT
CATCAGATGAAATTATTTTAATCTTTTCACGATAATATTCATCATCGCCGTAAGTTTCTA
CTGTTAAATCCCAATGACGAATTAGATATTCTCCAGAAACACCACGAGCTTTAACAAATG
TTGATGTATATTCTGCTTCTATAATACGAACTAATTGTCCGTTATTAAAAATAATTTCTG
ACACAGGCTTTCCATCAATTTTATATGTTTTAATTAATGGTTCCTGCATTACAATAATTT
CACCGACAATAAAATCTTTATCAGTTTCAAAAATCTTTTTACGAATAATGCTATTTAACT
TATCAACAGATTTATTCGTAAATGCCATTACACGATTTTCAAACAAATCATCTAGTGATT
TGACGATTGAAAAATAATTTACCATAAAATCGCGTAAAGCGGTATCACCAATAAATCCAC
GTACTCCATGCCCGTCAACAATTTTATCATAAATCCACTTACCGTTGCGAACATCAGTAG
CTACATCAATAATAGGAGCATTACTGCGTTTAACTTCAGTGAGTTCACATTGATAAAAAT
CTTTGTGCGTGAAGAATGGACTGATATAAGCAGTGTTTTCTCCTGGGTCAACAGGTCTAA
TTTGCTTATTATCGCCTATTCCAATTATAGTACACCAAGGTGGAATAGTTGAAAGCAGAA
TTTTAAATAGCTTTCTATCATACATTGACACTTCGTCGCAGATTAATACCCTGCATTTAG
CTAAATCTGGTACTTCTTTTTGTTCAAAAAGAACGTTTTCTTCATATGTTACTGGGTTAA
TTTTAAGAATGCTGTGAATAGTACTTGCTTCTTTCCCAGATAGTTTTGAAAGAATCTTTT
TAGCTGCATGAGTAGGAGCTGCTAAAATAATACCAGTTTCACCTGTAGATATTAAAGCTT
CAATGATGAACTTAGTAAGAGTAGTCTTACCGGTACCAGCAGGTCCATTAATAGTTACAT
GATGTTTCTTTTCTTTAATAGCCCTCATAACAATGTTAAAGGCATTTTTCTGGCCTTCTG
TCAAATCATCAAATGTCATCGGAAAGTCCCTGCAATTGGAATACTAACAATACGCCCAGT
ATCTAAAATTCGCTGATATAATCTTCGTGTGTCTACATCAGGCTTAACATGTTTAACTTC
TATTTTATTAAACCAAAATTTACGTGGAGTCTCAACTAATCTTGGAATTCCCTTACCTAA
AGCTAGTCGATACTGCTCTTTAAGAGTGGTAAATACTTTATCAGCAATCTTCCATTCAAA
AAATATAGCAGGACGATGTTCATCAAGCGGAACTGGCGCCGTAAATCCGTCTTTGTCTCG
GTAAACTATCGCATATACATAAACCATATTATCCTCGGATAAGTTTAAAAATTGAACAAT
TTAGCGGATATCCTCTTTTCAGTTTAAGTTTATCAATAAAAGACAAGTTTTGATACCGCT
CTACACCTTGAATAATTTTATCACACATATCATACTGCATTTCTGCTTCTGACAACTTTT
TCACAATTTTCCAATCCGAGCCTTTAAGAAGAACGTTCAATTTAACAACTTCAGCGCCTT
CTGCTATACGAGAACCATCAATACGAGCTTTAAGTGCTATAATCGTTAGCTTAACGTCAG
AAGTTTGTTTTGATTTAGAAAGCTGAGAAATGTGCTCAATTCGGTTTTCACGTTTTTTCT
GTATAGCTTTAATTTGATTATAAGTCTTTTTGATTTTAGCCCATTTCTTTTCATCTAAAT
TTAGTTTATGAACTTTTTTCGCAGATGAACGACCAATTCGCAAAGCAAATAAATCACGCT
TTTCAATCAACTCTTCTAAAGTATAATCAGAACGAAATGTATTGTACTTTTTCTTTACTG
CAATAACATTCCCTTTAATGTATCCAACGTTATTATCAAAACGTTCTAATGATAATTTCT
CTCCTTCAATACGATTATCAAAAGGTTCTCCTGAGTAAGCGCAAACTTTCTGATCTAAAA
TGTTCTTAATGTAATTGAAGTCTAAGTTAAAATCTTTAGAACGTCTTTTGGCAGATGCCT
GAGTATGCTCTAGACGACGTTTAATTTTACGAATTTGATTATTAGACAGTTTCATATTTT
TCTCACATCTTACGGACGATTTAATACTTTATTACTATAACATTTTTACCTTAACTTGTA
AACAACTTTATGTAAAAATGCTTTAAAACTTTCGTGGTATAATGAATCTAAGTCCTTCCA
TTATAGATTAAATCCTTCAAAATCAAGAGTATAGATAGTGTATGTTGAATACTTTTTGTA
CTCATATCTATCTGCAATTCTAAATACACTTCCAGCTGGTATCATTACTTCTTGTTCATC
TGAAACTAATTCCATATTACGATAGCGATGGCTATCCGGAAACTTAAAACTTGGACTATA
TTCTTTACAACGTAGAGCTTTTATAGCATACTCCTGGAAATTAAATACCATAGGAGCTTT
GAATTCAAAAATAACTTGCGTATTGTATTCTAATCCAGAAGCAAAATGTAGAGCTATATT
TTTATCATATGAAGCTGATACGACTTTATCAAATGTAATAATATCAATTCCTTGATTTAA
CACCTGCTTAGTCTCAGCTGGAACACCTCTCCAAAGAGGTTTATCATTTGGTTCCAAACG
AGATTTGATTATTTCATTTAACCAAGTATGGTCATCTGGTTTGTTGGTAATACAATGAAT
TAAAAGTTCAATTTCAGATAAATTAAACCCTTCAGAAAGTAATTCTTCACGAATAGAAGC
ACGCACCGATGCATCCATTGATTTTATTTTAAAATCTTTTAGTTGCATTACTGAGTATTT
CATTCAACTACCTCAATATCATAAACTTTAAATGTTCCAAATGAATCGTGTAATTTTTCT
TTTGAAATAGAAGTTATTTTATACTTTCCAATTGGAATCATCCATTCTTGTTCATGTACA
ATCATCATTAAGTTATCAGTACGCTCTGAATCTAATCCATCAGTATCTTCATACGTATAC
TTAAACTCAGTATTAGGAGAAGAAAGTATAATATCACTGATATGGTCAGAGTAATTAAAA
GCTTTATCAGTTTTTAAGCGAAGTACTATTTCAGTGAAATATTCAGCATAAGAAAAAGAA
CACGCTGTATGCAAACTAGTAGTAAATGAATCTACCCTGTTCGTTGAAAACACTTCTCCA
ACTTGTAAATCTTTAATAAGTTCTTTTGTCGATTTTGATATACCACGATATAGCTGATAA
GGTGATTTAGTTAAATGCTTTTTAATGATTTTATTCAATTGCCGATGAAGAGCTTCATTC
TTTTTAGCTTCCATACATTGCCAAAGAACAGACTGCTCAAAATCAGTAAATTTTTCACAG
ACCTTTTTATACATATCATATTGAAAATCAACGCTTTCAGCTTTTATAGATAACTGTTCA
ACATCTGCAAGATTAATAATCATGATAGCCTCCGTATACTTCAGAAGCTATCATATCATC
GTTAGAAAGGAAAGTAAACAACTTTTTGAATTATTTTGCCCAGGGAGCCCAAGGCGGAGG
GTCAAGATGGTATGAAGCTAGTTCTTCTAGAAGAGCATCTGGGGCTTCAATTCCATAATT
CTGTAATACTATACGGTACTCTTTCTTATAATTACTAGAATCATTCTGGATATTCGTAGA
ATGATTATCTTCTAACATCTCAAATAAATCCATATTAATTCCTAGCGATAAAAACCAAAA
TGACGATTAGCTTCAATAATCTTTCTTTCTTCTTCTGACATTCTCCAGCGTAGTCCAACA
TCAAAATGAGCCCAGACCATCTTAACAAATGCATTTATATCTTTAATATCTTCAATAATG
AATGTTTTACTTTTAGAAGGTTGACTAACTAATTTAACTTTATCGTCTTCAAACAGGTCA
AAAAGACCATATTCATGAGCTCTTTTATAGCCTTTAATAGTTAAAGCTTCGGAAGAAGAA
AACGGTGAATCAGTATTCTGCAAAATATCTTCAGTATGCTTTATAATTAGAATAACATTT
TCTGGATATTTTCTTTCTTTTATATCTTTAATTAAAATATCCGGATTTTCTGCTAAAGGA
ATAATTAATGAGCATTTTTTACCAAGAGTATTTACTGATTTACCTTCTTTAGACATGAAT
TCTACTGAATAATGTTTTGCTACTTCAATCATGTGATTTCCTTTTGCCTACTAATGGACC
ATCGGGAATTTTGTTTTCCTGGATATATTTCTCATTTTCTTCCATCATTTTACTACCGAT
TTTAAGAAGCAAATCCAACGCTTCATTTGCTTTAGCCTTGGCCTCTTCTAACGTCATATC
TCTGTTCACGATTTATCCCCATAGATGTCTCTCATCAATTTAAGCGCTGAGCGTTCTAGT
TTCTTTTCTTTCTCAGCACTAATCATTGATTTCATCCATTCTTCTGATTCGTTCTGCATT
TCTTTATTTGCCTGTTCAACCCAACCGTCATCAATATACATTGAGTTTGGTCTATTGAAC
CATTCAAGAATCTTCTTGAGAGCTTTCATTTATTTTACCTAAAACAATAGTATGGACACC
GTCAAATTCATGAATTTTTAGCAAATTTGGATTTAAATTATTCCATAAAGAGGTAATAAA
ATATGATAGCGCACTTTCGTCCGTAATTATAAATTTATTTCCTTTATCTATTTTCCAATC
ATATATTGAATCATATGAATAGAAGGATAACGGTTTATTATTATAATATGCTTCAGCAAC
ATCAATATAGTTAGATTTAGTAAATGCTTGAATTGCCATAAATGGAGAATTTTTCACAGT
TTCAATAATTCCGATCTTTTTAAGTTTTATTTCAACATCTTCTGGAACTGGCATTGAAAT
AAAATCTTCTACTAGATACATGTCATTGTCATATCTTTTATCAATCATTACTGCTACTGT
AATTGGAACATCCTTGACAAACGCCATACTAATACTATTGATAGACATTTCAAACAAAAT
TGCTTCCATAATTTTCCTCAATCACAAGATGTAGATGAACAACTAGAATCACAAGAACTT
CCACATGAATCGCCCGTCCATACATGAACAGGAACATTAGTATCATATGAATCAGAACTA
GATTGTGTATTCTGTGTGTTAGATGATGTAGTAGGTACTGACCAGCGCCAAGGATTTTTA
TAATATTCTTGGGCTTCTTCATAAGTCATAGTAACTGCTTCGACTGTTCCATCTCCCATA
TAAACATACTCAACTACTGTTAAAGGAAGATAGTCATTTGAAATAGGAACTACACCTTCT
CCAGGAGTTGTAGAGAAAAAATCCGTAAAGAAACTTTTAAACCAATTAAATATAAACATT
ATAAAAAGCCTCTTTGAAATTCGACTCTTTGAAATCCACCCCAGTCAGTGTATCGCATTT
CAGCATTGAACTCGACTGAACCATCTACATGCATCATGAATGAATGCGTGACGACATTAC
AATCCCAGCTTTTCATACCCCATTTGTCAGAGATAGCTTTATCAAGAACCTTCTGGAGGT
CTTCATTTGGTTTAATCCAACGATTTAACATAGTGCTCTCCTCTATAAGATAATTCTATT
ATACCATACTCATTGTGGAAAGTAAACCGTTTAAATGAAAAAAGGACTCCCGAAAGAGTC
CTTGAATTATTAACCGCCAGTTACTTTCCACAAATCTTCGTTTGCAGCAATCCACGCAGC
ACGTTGATTTTCTTCATATACAGTAGTATATGCTGCTTTTTCTGAAGGGAATGTCTGGTA
ATGAGCGCCAGAAATTTTGTGAACGTCAGAATAAAGAGGGAAAGCTACTGAAATTTCCTT
TCCTTCAATTTTCTTATTTCCATCTAATTTCTGCTCAAATGTTTTGATATTAACATAACC
GCGAGTACTAGCCATGTAATTCTCCTTTTATTTAAATTACATGATTATTTATAATAATTC
TCACGGCTTTTAAGTCGTCGTTTAAATAAACGAGCTATTAATAACCTCGGTCTTGACGAG
CGAAATTTTCGGCATTTTTCAGGTAATAAAGTTTAAAGATTTCTTCTGCTGTAAGACCAA
GACCCTGGAACATGTTCAGAACGAAATGAAGAATATCAATCATTTCGAATTTAATTTCCA
ACTGGTCTTGCGGAGACAAATCAGTAATTAGAGTTTCACGACGTTCACCATGTTGAGCTT
TCCAAGGCTTCCATACAGCAGATGCATCTTTTTCGCCATTGCTCATACCGCCAAGAGATG
TCAGAAGTTCACGGAATTCATCGTCAATATAATCTTTCTGATTACGTAGCCAATCAACAA
CTTCACCAGCAGTAGCCAAATCATCAGGATGACGGTTATATTCAGGTTTATCTTTAGCTA
AACGAACTTGCAGAGATTTTTGCATATCAAGCATAACTTGCAGCGGGTCTTTTTCATCAC
CGAGAATATCCCAGTATTCATTTTGAGCTTTATCAGCGCCTTCGATCAGTTGACTACATT
CGTTAAAGTAAGCCATTATTTTTCCTTTCAATTCATGGGTTAGTAGATTAATTATACAAT
AAATATATAAAGCAATAAGGAGGACATATGGTACAAAAATTAATGGCACTTGTTAATGCC
ATAAAAGGTAATAAAAAACGTATAGCTTTTACTATTTCTGCTATGGTAGGAATTTTACTC
TGGAACTTTATTCTATCACCTGTTGCAATTGCACATGGTATTAATATTCCAGTAGTTACT
CTTGATACATTTGTAGATTTAGCATTTGCTTTAGTTGGGTTAATTTAAATCTTAGCATAT
TTAGATAGCCGCATTTTAGCCATCAACCCCTGGGCAATATTATTTTTCATATATTCCATA
ATTTGTTCAGGGGTTGCACCTTCCTTTCTAATCATATCATTGACATCTTTTGATTTCCAG
GGAGATTTATCCCAAAACATAACCCTTTCTCCTGCATCAACTAATTTAGTCATTCGTTTG
ATAGTGTCAGGATGACGAGGTTCATTATCTAAAACCCACACACGTCTATCTTTAAATGGA
ACAACTTCTAGGTCTAATTGACCACCCGTAATAGCTATACCATTTTCAATAAAAAGTGAA
TCTATAGGTCCTTCTAGAACATATACATCACCATCTTTAACTCGTTCGACTCCATAGATT
TTTGTTGCCTCAGGATAAGCTTTGATGGTGATATATTTTTGAGGAGCATCTTTCTTTAAT
GCACGCCCTTGAAACGACTCGGCTTTTCCATTAGCATTATAAATTGGAATAACAAGACGA
GGCTCAGGTATTTCCTTTTTATATGTTCCCGGTGCTATGCTATTAACTAATTTAGGCCAT
TCAGTTGTAAACCAAAGATATTTCCATTTATCCTTTGGAATACAACGAGCTTTTACGTAT
TTTATAATTGGATGGTCTTCCGCCAGTTTATCTAATCTAACACATGACGGAAGAGATTTA
ATTATTTTCTTCTCAGGTTGTTTAGGAAGTTCTTTAGGTTTTTCTACTGAACGACTTTTA
CCTTTTTCTTTTCTTATTTCAAAGATATACTCACGATATAAATCAGGTTCAAACTCCTTT
AAATATATTCCGATTGGTGCATGATAGTTACAGTTATAACAATGAATATTTCCTTCATTA
TTATCGCCGTAATACCATCCACGGGCTTTATTTTGGTCGGTTTTTGAATCTCCACAAACA
GGGCATCTAAACCGTAATTTAAAAGTTGAACTATTATTTACTTGTGTGAATTTCGGTAAA
TGAGCTAATGCACGGTATGCAAACTCATTATCAATCCAAGGTATTGATGACATTTTTACT
CTTCTTTTTCTTTAGATTCCTCTTTTTTCTTTTTAGGAATCTGTTCAGGACCTTTATTTA
CTACAGCACCTGATGTTGTTCCAGTGGAGATATTTTCAGGATTACCGCCTGAATCTCCAG
CTACCATATCTTCTTTGATAAATTCTTTATATGTTTTCATATTAACCTCTATTCATAAAA
GCATTAAAAATTTGGTCATCAATAGATGGCGCAGTAATATTTTCTGCATCATTTAGCAAA
TCATTAATTTCATCAGATAGCATAACACATGTACCTTTATTTCCTGTAATTGTTACTGCA
CAATTACGAACGTTTTCTTTATCAAATTTTAGGTTAATATTTTCGTAGTGAAAATCCTTT
AATGCTGAATATATTTTTAAAATGAAATTGTCTTTTACTACAATAAACCCAACCTCACTA
TTAAAACGTGGTTCATGGAAACTGTAATAAACCTTTTCATCTATAAAATCGTATGAAATT
TTTTCCATTTTTAAATATGAAAATTCAGAACTATACATATTAACCTTTATTCATAAAAGC
ATTAAAAATTTGATCGTCAATAGAAACTTTTACTTTAGGCTGTTCTTCAAACGGTAATTC
ATATCCACATATTACAATTTTGTGATCGATACCAAAATACGCAGAAGCAATATGATTAAT
ATTATTTTCAGTGAAATCTAAATCAACATCAATATCTTTTTGACTAACGCTCAAAGGATA
ATTAATACGAATAATTCGATTATCTTTAACAAAGATTCCACCGACATACTCTGTGTTGCG
TTTAAAGTTTACACGTTTCCTAAATGAAAAATATTCAGGTTCTTTATGAGCCCGACTCAT
AGGACACAATGAGTAGCTAGAATAAGAGATGTCAAATCCTACACCTTCAATATGAGCCAA
CGCGTCATGATTAAACCAATTATAATCATATGCCAAATCCATTATATTGTAATATGTGAA
AGGCACCGGATTAACATCATGTGTTGTACCTTTAAAATCTGCCCAGGCAACGATTTCGTT
ATCTTTAACAATTACAAAAATTGTGCCAATAAGATATCCACGGTTATTTTCATCAGGCGT
TATATTATAACATTGTGCATCAGGAAATTTACCGTATTTAAACGGTGGATTTTTGTGCAT
ATAATAAATCATATTATTCACCTGTGATTTCGGTTACGATATTTTTGTTATTAAAGTTTT
TATCGCAATACAGAACATAATTATACTGCATTACACCACCAGACTTAAGCTGTTTTTGCA
CTTCAGCTTTCATTTCAGGACGATCGCGCTCAACGATATTCATAATATCCGCTTCAATTT
GAGTTTCAACTTCAGTCTGATCCGCAGTCATAGACCATTCACACAAATCTTTATCATAAC
CTGCCATAGCAGGCTGAGCAGCACAAGAAGCCAAAGCAAAAATTGCAGCAAAGATAAATT
TTTTCATGATAATCTCCTCAGTAGTTTATGTTTATATAGTATCTCAATTTCCAACAAAAG
TAAATAGTTATTTTAAAACTTCTGCGTAATCACATGTTACAAACTGTTTCTCTAGCTTGA
CGATTTTACGAAAGTATCTTTTGCATTGACGAATCTGCCGCTTCGTAGGACGTACAACAA
ACTTAATAAATTCCACTCGACCAAATGGAGGACTTTCTTCTGCTGGAATATCTAACACCA
ATTCCCACGTATCTGCAATAAGTGCTTTGAATTGCGTATTTTTCCTGACGTTATACGGAG
TAGGTTTAAATAAAACAATATGCATATTATCCTCGGCAATCTACTTCACATACTTTCTTG
TCATCAATGAAAGCTTTAACTAATGCTTTATTAACTTCAGCATATTGAGTAGTGGCCCAT
TGAACGTCATTTTTCATCATTGTGGTTTCTTTAGTAAACATGCTTTCATTCTTAAACCAC
CCCATAAAAACTACCTTTACCAATTCCATAACAATATCCTCATTTAACCAACAAGACTAC
TATACCATAGTCTCGTCAGCTTGTAAACTAAAATTTTAATTCATTTGCCAAAGCATCTAA
CTGAGCTCGAGTAGATTCATTTCTTTGATAGCGATTCTGTTCAGCCTGAATCTGTTGTGA
ACCTGCTACTTCACTCACTTCAGTTGGAGTAGAATCTTGTTCAATTTCTACCCATTTTTG
ATTTCCTTTTTGAACACCCATCAAAAACTTATTCCACTTATTCTTATCGCCATATCGTGA
TTTGATTTGCTTAATGAGTTGTTGTTCAGCAGCTGCTAGCTCCTCGGTTTCAATGACCGC
AAGCATAAAATCGGCTGTTGCTGGAAGACCAGCAGATTCTGCAATATCGCTCATGTTAAC
ATCGGAAGAGTCCCAAGCTTGTTTACCAACCTGCGCTGCAGTCCAAAGAACAGTTTCGGT
TTCAACAGCAAGAGCACGCAATTCCTCTGCAATAGCTTTAACAGTTGTGTAACTATTTTC
TGAATAAACTCTGATGCGACAAGATTTACAAATACCTAGATAGTCGACAATAATGATTGT
TGGAACAAAATTCTTTTTGAGCTTCAATTCATTCAAGAGAGACCTGAATGTGTTAGCATC
TGCCCCACCAGTAGGGTACTGTTTAACGATTAAACGACCGAGAGTAGATTTCTCACGCCA
TTTTTCCATTTTTCCTTTATACTCAGCGTAAGAAATATGCCCATCATCAATGTCATCAAG
AGAAACATCAAGCATATTAGCGTCAATACGCTTAGCACAGACTTCTTCTGCCATTTCCAT
GGAAATGTAAAGAACATTGTGTCCGAGCTGTAAATAATCTGCTGCTAATGAACACAATCC
TAATGACTTACCAACGTTAACGCCAGCCATTAAAACGTTCAGCGTTCCAGTCTCAGCTCC
GCCTTTAGTAATTTTGTTTAGAATTCTGAGTTTAAATGGAACCTTACGAGCTTTATTCAT
ATAAGATAGCCAACGTGCTTCGTAGTCATCCATCCAATCATGACCAACGTAACTATCAAA
TGAAATAGATAATGCCTGGCGCATGATGTCAGGAATAGCACCAACATCCGGCATTTTCTT
ATTTCGTTTTTCCGGAGGAAGCTCAGCATTAGTTTGAATTTCGATTATTTTAGACGTAGC
ATTAAACATCGCCCTTTGCTGAACATATTTTTCTGTTTCTTTTACTAACCAACTGTGGTC
TTCTGGAGAATCAGTTAGTTTTGAAATAAGTGTTTTTACACCAGAATATTCTGTTTCAGT
AAATGAACTATTTTCTAATGCAACATTTAACGCATTAATAGATGGAACGCTATGGTACTC
ATTAACATGAGATTTAATTAATTTGAATGTATTTTTAGCTGGACCACTTTCAAAATATTC
TGAATCCATATATGGCCAAACTTTTGAAAAATAAGCTTGATCAAATATGAGATGAGAAAG
AATAATTTCTACCACACTTACTCCTTAAAAGAATTTAAATTTTTTCTTTGACCTTTTATT
AAATGCATCTTGCAGTTGCATTGTAATACATTTTTCTACATGAGGAGCTAATTCAGCTTT
TCTTTCTTGGTCAAGAACAGCAAAGTCCATTACAACCTTTCCATCAACCCAATCCAGTTT
TGTTACATATACTATATGCGTAGAACCATCTTCTAGTTTAATGACAATCTCCTGGATAAC
ATTTTCCATAGCGGATTTAATTATCTTAAGAGACTCATTAAAAAGACGTTCTTTTCTTTC
TTCTTCCCCCTCCGAAGAGGGGGATTCATCGATAATTTCTAGATCTAAATCTAAATCATC
TTTATTCATTAAATTCTTCCATATCACTTAGCTGATCTAGGTCAGTTTCTAAATCAGCTG
CTGATTTACTTTTACTTTCTGGAGATTTAAATTTTTCAACCTTTGAGTTAATCAATTCAT
CAACTTCAGCTTCAACAATTTCATTACTATCAATAGCACCTAACTGATAAGCACGCTTAA
TAGCATCACGGAATGGTTGATGCTTAAATAAAGGACCCCAGAATGTAGTGCAGTTGGTAT
CTTTTGCACGCCAAGATTTTTCTTCGCGAATCATCTCGCCAGTTTCTTCGTCAAGAAATT
CACGAGCATACCAGCCATTTTTAGGTTTTACAACAAATCCTAATTCTAGAGCCATATCTA
ATAATCCAGAATAAGGATCGATACCACCGTCAAATTTAACATCAATAAAGAATTTACTTT
TTTCTTTAACGGTACGAGATTTTTCTACGTTTAGAACAAATTGATACCCCTGAAGATCAG
AACCATCTTTAATCTGGCGTTTACCGATAATGAATACGGTATCAGCCGAATACATTACGC
CTGTACCACCTGTCATCACGGTTTTACTAAACATTTCAATTGTTTCAATTGTATGGTTAA
CCGCAACACATGGAATATTTTTAATGCTAAAATAAGGAGTAACAATACGGAATAATGACT
TCAGTGATTTAGCACGAGTCATATCTGCCACAGATTTTTCATTCAAGGCATCTTCCGTTT
CTTTCTTAGAAGCCATATTACCGATTGAGTCGATGAATACTATAACCTTTTCACCACGCT
CAATAGCTTCAAGCTGATTCACCATATCAATTTTCAGCTGTTCAACTGACTGAATCGGCG
TATGAATTACTCGTTCCGGGTCAACTCCCATGGATCGCAAATAAGCTGGAGTAATACCAA
ATTCGCTATCATAGAATAGACAAACCGCGTCAGGATATTTGTTCAAATATGCCGCAACCA
TAGTCAAAGACATATTTGATTTAAAGTGTTTAGAAGGCCCTGCGAAAATAGTTAAACCAG
ACTGCATACCGCCATCAATCGCACCAGAAATAGCAATATTAAGCATTGGGATTTTTGTAC
GGATTACATCCTTTTCATTAAAGAATTTAGATGTAGTCAGTTCAGCAGTCATTTTAGAAG
TGGAAGCTTTAATCAAACGGGATTTTAAATCTGCAATAGACATTCATTTTTTCCATAGGC
ATCATTATATTTTCCTCACTGGTTAAAGATAGAGTAATTATAACACAATAAATTTAGGCA
TTAATCAACTGCTATCGGATGAATAGCATTAAACTTATGAAATGCTTCTGATTTTTCTTT
ACGCGAAACACACATGCGAAGAACCTTTAGTGGTTCATCTTCTTCACCCAACGATTTTCG
TTTTTCAATATTAGAAGTTTTCCAACGAGCTTGTTCTGGAAACTCTCTATTGATTTGCTC
AAGCGCTCTATTATGTTTAGAATTACTACGAATTGAACTGCACCCGCCAGGAGCTTGTGC
TTTTCCAGATACAACCAGATATTTGAACAAGGCCAAATGCGGATAACCTTGATTAATTAA
ATTGAGAAATGCATACATATCTTCGCATAAATCAATTTTTCCATACCCAATTTGTTCTGT
CGTAAGTTTTCCGAGGTCATACCATGTATTCGTGAATCCGTATGAATTTTCACGATAATT
ACCCCAAGATGAAGTAATTTTAAAAATTGGTAAACGAGCATGACCATGATAATACCCGCA
ATCCATGGCATCCTCAACATATTGAATCAATTCATAGAACTGTTCACGAGTCAATTGATT
AATTTTATCTACACAACGACGATCATCTCTTTTTCGCATTGAACTCATACGAATAGTAGT
ATCATCGTCAATCATCCAGATTCGTTGACCTGCATACATATCAGTAATTGCTTTACGAGT
TCCAGCAATTCCATTTACATCATCAGGAATAGTTATAATTTTAGCTCTAGACCCATAGGC
ATCATAATAAGCTTTTTCTTCGTGTTCACGTACTACAATATGTGGTTCATAATCAGTCGG
AAACATATCAAGGGCAGAAACTGCCCCTACACGTTGATAGCTTGGAATTACGAATTGAAT
CATTTCCACTCACCGTTATAATCTTTTTTCACAATATGAGTTTCGCCAGTATTCCACCAA
TGATCAACCAAGTAAAAATGACGAGAATATACATGAAGACTTCCAACATTCCATATAATA
GAACCTGCTTTATACTGACGAGTTGAATCGCCTGCATTCAAATCAGATACTAATTTATCT
AGTACGTATTTTTGCCATGCATAATCATTACGGAATCCGAAGACCACGTCATTTGAGCGC
ATGCTTACTACTGCATTGACTTTCTTATCACGAATCAGGTACTGTACTGTATTAGTACAC
ATAAAATCTGACATACCATCTTTATTGTAGTCAAATTGCATGGATGGACGAGTATAAATC
ATGATACCACGTCGAGAATCAGGATTTTGACCAAGTTCAGCTAAACACATATCATACTGG
GCATAGTTATCTTCTGACCAAATAGCCCAACCATAATTCGAGTTAATTTCGCCTTTAGAA
GATGCTACCTGCTGCCAAATCTTTGGTGTTTCACCTGGAATATCTTTAACAAACAAGCTC
TTAGATTTATACCATTCAAGTTCACGCTGAATGTATTCATCATTAAGAGCGCCAAAAATA
AACGGTTCATCTGCTACAAATGATGCGCCAATAATTTCAATAGTTTTAACACCAGTTTTA
TCAACTACGAAATCTTTTTCCTTTAATGCAGTTCCCAAATAAAAACGGATTTCTTCAACT
GTCATAGAGTCATTAATCATTTAAACCTCAATTGATGCATTCATATTTAAATTGTAACAG
TAATAAACCCCAACCTAAAATAATAGTTGGAATCATAAGAGGAACCGTTACACTATAGTA
TATACTTATTATAATCATCAAGATTAAAAGCAACACTGTTATAATTTTGCTTTTCATTCC
TTCTCTCTGATGATAATTACCTGATTTGGTTGTGCAGACTTTTTAGTTTCACCTGCAATT
GACCAAATAAATGTAATAAACCAACCAATAATTGACCAGTTAAACAGTAAAGATGTGAAA
AAGATTCCTACTGTCGATTTTGACCCACGCATCAAGGCGATAAACCATGGAAGCATGTAT
ATAATAATAGCCAACACGCCTGAAACTAAAACCATAAAAATTGAACCTGCTACTAAAGTT
TCCATGTTTTCCTCACTTAGTCAAATTTTTTACGCATGAATTATAAGAATTCACTACATA
CTCCATCGGAGCATTTTTACCAGTACGCCATTGGTAATTATTAGCCCAATTAGCCCAAAG
CTCGGCGCAGTAGTTTTCAATTTTTTCTTCGCGTGTAATTACATCAGAATTACGATATGC
TTGAGCAGATTCATCTGGACGAATAGCTTCGTCAAAATTCGCCTGCATTTGTTCTACTGT
CTGTTTTGGAGCTTCTTTATAACACTTGACATTAGGATTATAAAATTTGCTTGAACAGTT
TACAATTTTTCCTACATCAGACTGATTTACTACCGGTCCTTGAGCTACACAACCAGTAAG
ACCTAATGCAATAACCAAAATAGCGATTTTCATTTCATTCTCCAAATCCGTATCAGTTGA
TAGTTGTATAGTACCACGGAAGAACAGTCTTGTAAACAGTTTTGTGAAAAAATTTTTTAA
TGAATCCAAAAGGTCCAGAATCATCTGTTTTTCATAAGTATAGATTTATATTACTTGTGT
GAAAAAGGGACCCGGAGGTCCCTAGATTTATTCTATCAGCCAAACAGGAAGTCTAACGAA
GCTTTTTCTTCATAGTCCATACCAGCCGATTCACACATACCCGCAAGCGGTTTAACAAAC
GATTTTTGGAACAAAGTTGAGTAGTCAATCCAAGACAATACGTCAGAACGAATTTCTTTT
GGAAGTTCTGTTCCCGACGGCCAAGCAATGCACTTGTCACCAAATGGATTTCCTTCACGC
AATGGAAGAACCATTACCTTATTTCCATCCAAAATTGGAGCTACACCTAAACCGCTAACA
GCTCGACGATAAGTTAGCACACCACGAATATGGAACGGACATTTAAATCCTGGCCAACCT
TTATCATCATATTTCGCTATATCGTTCGCAGTTTTTACTTCAGCAATAACTTTATAGTCG
AGTTGACGATATTCTTTCTCGAAGTTCTTGTAGTATTCTTGGACAGACTCTTCGCCTTCC
TGAAGAATACGACGAATACTTTCTTCGAGAGCTTCTTGTACTGCTTTTGGTGTTGAACTC
TGCTGAGTTTCCATACCCATGATTTTTAGATGTGGTTCAGCAAATCGCTTATCTTCCATA
TCATAAACGTTCAGAGCATAACGTTTTTTCGCTTTCCAAAATCCACCAACACCCTTTGAA
CCAAGCGGAGGACAAGAAATAGCTTCACGGTCCATATGCATCAGATGCTCGCGGTTATTC
ATATAATCACATAACTCACGATATGCAACATCAATCATAGGTTCCATCTTTTTCTTACCG
AACTGATTCATGAATTCAACTAAATCATTTTGTTCTTTAAAGCGGTCAAGACCTACTTTT
TCAATAACTTTATCTACACAAACATATACCGAATCAGTATCACCTGCTGCGATGAAATCT
TCACCATCGGTTCCACATACTTTATTCAGATATTCATTAATTTTACGAGCAATCCACTGA
ATACCAACTTGACCAAAAATTGTGATAGCAGTAGCATTTCGCAAATCATAATAGCGGAAA
TGAATGTTACCAAGAGCACCATAAAGACTGTTAATAAGAATTTTACGGTTCAGCTGATTT
GTATTAGCAAGTGTAGCCGCTTTTTCACATTCTTCAATCAAACTATTGAGAACAGATTCA
GTATAATTCGATAGTTCATTTAAGAAATCATCACTGAACTTAACATATCGTTCAACTTCA
GGTTTAGTTGAACAAGACCCTGCGCCTTTCATAATAATCTTTTTAATAGCTTCGGCATTC
ATTTCTTCAGCGAACATTTTCTTTTTCCAATCTTTACGCTGGAAAAATACTTTAGCGATT
TCCTTTGGAATGATACCTTCTTGATGCTTATCATACATCCATCCATTCGGAGAACAAGAA
TATTCATCACTCGGTTTAGGAGCTGTTCCTGCGATATATTCATGAATTGGATGAACTTTA
AACTGACCACGAATAGTCTCAGGACTAATGTTAACCTGACGAATAATGCTCGGATACAGA
GACGTCAAGTCAAAACTCATAATATATCGACGAGCAATTGGTTTAGGTTCAAACACAAAT
GCACCTGGAAAACTCTGTTTAACGTGCGAACCTTGTTGAGGAATAACCTTGTGTTCACTT
TTCAGTGAGTTAAAAATAATAGCATCCCAAGTTTTAATAGGACTCATTACACCAGAAAAA
GGCATTTTAGCGTAATAAGACATACTTAAAACTAGATCGATAAACCCGCGAATTTTATCA
ATTGCTTGAACTGATTCTACGTCAATGATGTTATAACTGATGTATCGTTGATGATTAGTC
TCACGAAGTTTATTAATAGGACCGTCGTATGGTAATTTGCCCTTTTTGGTTTCATGTTGA
GCAACTGATTCCAAAGAGAATGACGGCAAATTAGTAAAAGCGAATTTCTTGTACAAATCT
AAATAATCAAGAATAGATACGCCATCAATAGAATAAATTTCTTTGCTACCGTACATATTT
TGAATTAGTTTAGATTTTACCCGACCGATTGGAGAGAAGCGTTTCATACTACGTTCGCCC
AGAATCATTTTAACACGATTCATGATATATGGAACGTCAAATCCCTCAATATTCCAACCA
GTAAAAATAGCAGGTCGTTTCTGTTCCCAGAGATTGATATATTCCATGAGCATATCACGC
TCATTATCGAATGGCATATAAATTACTCGGTCAAGAATTTCTTGAGGAACTTCATCGCCA
CCTTCACAGTCAAGTTTAGCAGCTAACTTTGCATCCCATTTTGATACTGAACCGTACATT
GAATTCAACAAATCAAAGACGTAAAAACGATCGTCAATTGAATCATAATGAGTGATAGCA
TCAATTTCATATTCAGCTTTCATTGGGTCAGGAAATTTATCACCAGTAACCTCAATGTCA
CAGTTAGCTACACGAACAAATTTTCGGTCATAAACAATTTCTGAACCGTATGTATCACTG
ATATAAGCGAGTTTAAAATCGTTCATACCGAGAGCTTCGAGACCGATGTCTTCCATTCGT
TTCATCCAATCTCGAGCATCTTTCATTGATGGAAATTTTTGAGGAGCGCAATTTTTACCA
TAAATGTCTTTGTATTTTGATTCTTCCTTACAGTGTCTAAACATAGTTGGAAGATATTCT
ACTTCACGAGTACGTTCCTTTCCGTTTTCATCAATATAACGTTCAACAATGTTATTTCCG
ACTGTTTCGATAGAGATATAAAATTCTTTCATAGATATTCCTTAGTTTATAGCCCGAGTT
ATTAGGCTCTTGATATATTATACTCCAAATAAGGGGCCGAAGCCCCTTGCTTAATTACCA
ATCGTATATTTAGGAACGAGTTTCCATTCATGTTTTTGTTTAAAAGAAATAACTCGGAAG
TTATTAGTTAAATCTTTCATAAAAGTTCTTTGACCAGGAACAATTTCAATCAGTCCCCAA
TCTTCTAACAGCCATGCAATCGAATCACGACGAACTTCATCTTCTTCTGTCATTTCAACT
TGGCGACCATCCATACGAAGCATTTCTTTAAAATGAACGATATAGTATAGTCCTTTTTTC
TGGAGAATATGACAGGATTGATACAGAACTTTATCTTTATTATTAGCAATTCCCATACGA
GTCAAAGTTTCTTTAACTTTCAGAAAATCTTCAGGTTTTTTAAGAGTAATTTCAATCATT
TTACCATTCCAATGCTAGTTTTTTGAGTTGTTTCTGTTCTTTTACATTCTTAGTCACTTC
TTTCAAAAAATCATCCGTGACTAAACCTTTTAGTTCTTTTAATACTAAAGGAAGTTTTCC
ATTTTTAGTAAGAATTGATTTATAGTTAATTGCATCATTTGTATTAACTTGATACCGCTT
AGCAAGTAACTTAATAATCAATACTTCGGTAGAATCTTCAACCAGTTTTGCCCATTTACC
ATATCTTTTACCCCGAGGAACAGCAGCCATCAGATAATTAAAATGGGCTTCATCACTTAA
TCCAGAACCGATTAAATTCATTGCATACACTGCCGGCATGCACTCTGGAAATTGTGATAA
TGCATTTTCAACCATGAATTTTGAATAATCTTTTTGAGCAATAGAGCATTTAGTTTTATT
ATTAATAGCTCCAATTATTTCAAAAAACTCATTTTCAGCTTTTTCTTTAAAAGAATCAGC
AGCGGATTGGACAGCTGTCCAATCTTTTGAATACCAAGCAACTTGATGCTCGTTTAATTG
AATATCATCTTCAAATAAGCTCATATCACTTCCACTGCATTTCGCATGCCAATTGAATGA
AAAGATATGCTAAATGTAACTCAGTATTAGCCGCAATTCCATGATACTGGTTATTTTCAC
CAACAATTTCATACATACGAATAATACTTTGCGGAGTTACACGTGAATAAATTTCTTCGG
CAAGTTTACCTACGAACCACGAATAATCAGCTGCATATTTTGGTGCTAAAGCTCTGAGTT
GTTTAACATCTTTATTTTTGAGAGACTCAAGAACATCATCAATAGCACCACGATCGTTAG
TAACCAGTGATAAAATACCAGCATCCAAAACACCTTTAGATGAATAACTATCGAGCTCAC
CAATAGTTTTACGAAAATCAGGAAAATTCTTTTTAACCAAAGCTGCTACAACTTTCATAT
CAGCTATAGCAATTCCTTCACGCTTGCAGATTTCAGTCAATCGACGAATCATCTGCTTCA
TCATTTCAATTTTATCTTCATCGGTTGGTTGACCGAACGTAATAACTCGGCAGCGTGACT
GAAGCGGTTTAATAATACCATCAATATTGTTAGCAGTAATAATAATACTACAGTTTGAAC
TATAAGCTTCCATAAAGGAACGAAGATGTCGCTGAGACTCTGCTAACCCTGAACGGTCAA
ATTCATCAATAACGATTACTTTTTGACGACCATCAAATGAAGCTGCGCTAGCAAAATTAG
TCAAAGGACCGCGAACGAAATCAATTTTACAATCTGACCCATTCACAAACATCATATCAG
CATTTACGTCATGACACAATGCTTTTGCTACGGTTGTTTTACCTGTTCCTGGAGAAGGAG
AATGAAGAATAATATGTGGAATCTTACCTTTACTTGTAATAGATTTAAAGGTTTCTTTAT
CAAAGGCGGGAAGAATACATTCATCGATAGTAGATGGACGATATTTCTGTTCAAGAATGT
GTTCTTTTTCATTTACAGTAATCATAATTTCCTCATTCAAGTTTTAGTGTAAATTATAAA
GGGCCGAAGCCCTTTATTAAAAGTCGTGGGTAGAATCAGCTTCAAGAGCTACTACATAAT
TCGCATGTTCACCTTCAAATTTAGCAGCGCCTTGTTTACCTTTTGCCCAGAGCAGCAGTT
TATAATTTCCTGGTTGCATTTTCATATTTGCCATATTGATAATGAAATTAAATGTATTTT
CACCATCATAATCGCCAAGAGTCAAAGAATATTTAACACGGGTCAAAGCAGAATCTTCTA
CTTTATTAAAACCGTTAATTACGATTTTACCTTCTTTTACCGTGATAGCAATTGTATCAA
TCTGCAGACCACGAGATACACGCAACAGTTGTTGAAGGTCTTCAGCTTTAATTTCAGTAA
CAACAGATGCTACCGGGAATGGAATTGGTTTATTAGGAGCAACTACTGTACTCGGATCGG
CTGCTGGCCAAAAAATTGTTGAGCGGGCATCAGCAATTTTAATATTTCCATCTTCTGACT
GTGAAATTTCTGCATCATCATTAACTAGAGACAGAATACCGAGAAAACCGTTCAAATCGT
AAATTGCTACATCAAAATCAATAACGTCAGAAATATTTGCTTCCGCATAAGTTGTACCAT
TAACTGCGCGAGTCATAATAAATTGACCGGATTTAAGCATAATACCGGAGTTAATAGTAG
CGAAATTTTTCAGCAGAGCAGTAGTATCTTTAGACAGTTTCATGTAATTTCCTTCAATTC
AAATGAGATTTAATTTTATAACTAATTTAATAAAGCAATTAACGATTAAAATCAGCTGCG
ATTGTTTCGGCAACAATTTGAGCAGCAACAATCAAACGTTCATCTGCATTGCCGCAATAT
TCATCTTCAAGGCGTTCACCACATGAAGTCATAATAAATTTAGCACCGGCGTTTAGGGAT
TCTGTAGTATGTTTGCGCATTAGTTCAATCCATTTATTACTTACTTCACGATCGATAGCT
TCATAATACGCATGACGAGCAGGTGCAGATTTAATTTTGTTCTGAATAACTTCCATTGCG
TTATCAGAAAGAGACAAAACCCATGCTCGACGAATTTTATTTTGGTTTTGTGGATTTGAT
TCAGAACGCACGTGTTTTGGCTGAATATCTTTTACATCAACAGTATAATTCACAGTAATT
TTAGTCATAATACACCTTTAGTCATAATAATCAGTAACAGTCCAAGCTTCATTTCTATTG
GACATTATTTTTGTATATTCTGCTTTAAATGCATTCCTAAGCATAGATTCAGTAACTATA
TGCTCTTCATTAGAAAAATTATTTCTCAGAATATATCGTTTTATTTCAGGAATAGTTAAT
AGATGTTGTCCAGTTGAATATTCCAATGTTTTTCCTCCATAGAGATTATACTCTAATAAA
TTAAAGCATAATCTCTTATAAATTAAATCATTACAGTAAATCGACCAACCTTCTTCATTT
GAAGATGCTGACCATATGCTTGCGGGTCATGGTCTCTATGTGAAATAACAAAAACATTAG
TATTTTTTAAACTATCTAAAATAGTTGAAATAGCTTTTACACCTTCAACGTCGGTCGCTG
AATCAAAAACTTCATCAAGAATTAGTGTGTTTATTTTAACACCTGAAACTTTTTCAGCAA
TATCACGCCAAGTAAATAAAAGAGCAATATCAATTCGTGCTTTTTCACCTTGACTGAATG
AAGCATAACTAAAATCTTCACGACCACGGGATTTAATTGTCTCATTAAATTCTTCATCTA
ATGTAAACACATAATCCGCTTCCATTATTTTAAGATAATGGTTAATCTGCTTATTAAATA
ATGGAATGTACTTTTTAATAATAGCACCTTTAATACCAGAATCTTTGAGCATATCAGTCA
AAATTCCTCGGTGGTATTTTTCCATTACTAAATTAGTTTTTGTCTTAACAATTTTATCAA
GTTCTTCTTGAAGCAGTGCTATTTCATCAGCATGGTCAATAAACTCAGAAGATGCTTTTT
CTATAGCTGCTTTAACTTTTTTAGCTTTATCTACCGTCGTGATTAGAGATTGCTTTTTAT
TGCGAATATCATTTGCCAACGACTGCTGGGTTTTAATATTATCTCGGTATTCATCAACAA
GAACTTTTAAATTATCACGATGTGTTGAAAGCTGTTCAAATGAATGCGTACATTCAGAAA
CTTTATCTTTAATTTTAGAAACAACTTTATCACCGGAACTTAATTGTGACAAACAGGTTG
GACATAATCCACCTTCGTGATACATATTAATGACTTTATTATACGAGTCAATTTTTGATT
TAATTAAAAATGCTTCTTGACCGATTTTATTAAATGCATCAGTCGGGTCTTCATCCAAAA
CAATATTAACTAATCTTTCATTAGCTTCTTCTATTTCCGATTTTAGCGTTCTAGCTTCTT
TTGCCAAATCATCATACATATTTTGCAGACGTGTAAGGTTGTCACCCGTTAATTTTTTCT
GGCGTTCAACGTTATCATTATATATTTTAATTTGTTGGATAATACTATCTTTTTTAACAT
CAAGCACTTGGTTTTGTGAATTTAATTCACGTATTAGTGCTTTATTAAGCTTATCCATTT
CAGCTAATGTTCCTACCTCGAGCAGGTCTTCCACAAGCTTTCTTCGCGCAGGGGTCGACA
AACCCATGAAAGGGGTATACCCTGCTGTACCAAGGACAACAATCTGCTTGAAACTGGCAT
ATGACATTCCGATAAGCTGTTCAAATTCTGCTTGGAAATCTTTACTGCTGGCAGATTCAT
TAAGACGTGTACCGTTAACGGTGATTTCGAAAACGTTTGGTTTTTGTCCTCTTTTGATAT
AGTACTTTTTCTCATCATATTCCATCCACAGTTCAACTAAAAGTTCTTTCTTATTTGTGC
TGTTTATTAATTGACCTTTCTTTACATCACGGAATGGCTTGCCAAAAAGCCCAAATGTGA
TGGCTTCTAACATAGTAGACTTACCACCGCCATTTCGTCCAGTAATAAGAGTTTTTTGAA
CCTTATCTAATTGAATGTCAATAGGATTTCCACCTACTGACATTATATTTTGATACCTAA
CTCGGTTTAGTTTAAAATTCTTCACAAAAGATTCCTTTTAATGTATCTTTTAGACCATTC
TATCATATCATCATAATCTAAAAAGTATTCATCAAATTCAGCCATGCAAACAACGCCTTG
TGCTGTTTTTGATGTAATATAAATTATTCCAACATATCTAGAATCTTCTTCGGTATAATC
AATGTTTGCTATGAATTCATCATTAATGTCAAATGTCGAAAACTTCACAGTATGCATCCT
TAATACAAGATACAGCCATATCTTGTAATGATTTAGGTGTGTCATTATCTAAAATGTTGA
AGTTAAAAGATACAGCCCAGTCAGTGCAGACCGTGACCTTTTTAATACAATTATCTTCAA
CCTCTAGCGGTTCAAACCAATATTCAATAATTTCTTTATGCCCAATATCATCTTTTACTT
CGCATTCAAAATGCTGACTCATCATAACATTTTTAAATTCATCAAAAGTCATTGTGTTGC
CTCTACATATAACTGATTTGCATATTGAATAAGTGCTTCGCGATCAGAATCAGTGATGTC
TGGAATTGCATTAATGTATTCTTCCATCAATGTCTGAAGAGATTGAACTTCAACTTCTTC
ACTATCATCTGACTCGACAGAGTTATCAATCTTTGACACAACTCGTAATGAATGCACAAC
TTTTTCTAGTTCAGATTCGAACTTCGTCAGATTTTTGTCTACTTCAGTTACTATAACACG
TACTGATAGATTTGTAAAATCTTTATAGTCAATTTTTCCTTTAAATGGATAATGAATTCT
ACGATGCCAGGTAGTATTGTTTGGAATAAATTCCATTCGTTCTGTTTCTGTATCAAACAC
CCAGAACCCACGAGGGTCATTCTCGTCACCTGCAGTTAGTGTCCATGGTGTCCCAATATA
TCTGACGTTAGCAGCCTCAGAAATAGTATGGAAGTGACCAGACCACACCTCTTTATAAGT
CTTAAGGAAATCAGGTTCAAGACCGTGAGATTTCATTCCTTTATAAAAATAAAATCCATT
CAGTTCCCAGTGACCAACACAAAAAGAAGCAGATGAAGTTTTGATATGCCCGAGAATTTC
GCCAGTATTTTCTTCACACATCCAAGGAATTAAATCAATTAAACATCCGTCAAAATCTAC
TGTAGTAGGCTTATCATATACTTTAACATTAGGATATTTAGCCAAAAGCTCAGTAGAAGC
ATTTGGATGCATTACATTTTTGTAGTGGAGATCGTGATTTCCTACAATAGTATGTAATGT
GATTCCAGCATCATCAAGCATTTGAACTATTTCACGGGCAAACTCCATAGTTTTATGCGT
GATCGCTTTTCGCACATCAAAAATATCACCGTATTGAATCCATACCGTAATTCCATTTTT
CTTAGAATATTCTATCGCTTGCTTAATTCCATCAATTTGAATACCGCGAATCCACTCATC
ATCAGCTTTAACGCCTAAATGCCAATCACCCAAGTTTAGTATTTTCATGGTGTCTTTTCC
ATCCTAATTTAGCTGCTTTTGAAACCAGTTCTTTAATTTTTCCTTTTGTCCATTCAGGAC
CAGGAGGTTCATGACATAACATTTGCTTAACTCCGTTATTATACCATTTTTTACCTTTAT
TATGTGATGAAGCTTTAAATTTATTTGACCTTCCTTTTACATAACCTTCTGGAATATAAG
GCAAAAAGGCAGATTTTCCTGTTAATGGATCATGTGAATGTATATTTCCTTTACAATTAT
GAGAACCATATAATGGATGATTTTGTCCTTTATTATCTCTTCCAAATGAACCTTCTGGAG
CAGCATATGACATATTAAAGAATAATGGATTTTTAACTGCATCATGCAATCGTTGATAAT
ATAACTCGCATTTAATAGCTTCTTCACGCGTATCAAATTCAGCTAAAATGCATGAATCAA
ATAAGTGAGGATTATCTGAAAGCTCTTTATTATAAATTTTCTTATACTTTTTAGATAAAA
CTGAACCATGATATCCAGCTAAATGTCTATCTAAAGACGTAGATCCAATATAAAAAGGAG
GAAGTTTATCTCCACTATATATTGTTAAATAAGTTATATATTTTTTCATATATCAAGAAC
CGTCATTGAAATGCAAAATAAAATTATTGAAATAAATCCATCTGGAGTACTAAAGAACCC
AATCCAACATGCTCTGGTGAATAGATAAAATGCAAGAAAAAGTATCACATATCCAAGAAA
TATCATTATATCAAACTCCGTATAAAGCTAAAGGGCCGAAGCCCTTTATTTTGTAATAAT
GTCAAACTGCTCTTTAAAGCAGAAGCTTGAATCTTGATGCTGATACAAAAATTCATAAGC
TTTTTCACGTTCGCGGTCATAAAGAGCTCGGTCAGATGACAGTTCTTTAATACGTTCAAA
TGTTGATTCCATATCATTTTCATCAAACCAAATGATACCGCTATCATGCGAGGTCAAAGG
AGTATTATCAACACGGAATTTTAAATTTTCGCCAGTAGATTTCCAAAATACTGGAATTGT
TCCACACGCGCCAAGCTCGAGATGAGTATATTCTAAAGAACGTTGTAAATATTTTTTGTC
CAACTTACTCAGCTGATAACCAAATCCGGATTTACTCATGCGTTCAAGCATTTCGCTATT
TACATAACGGTCAAGAATTTGCGTTGGCAAATTAGGAGCAATTTTAATTTGGTCTACTTG
ATGAAGACGATAATACTCGTATGGAATTCCTTTTTCTTTAATAGGAATGAACGCTGGAGA
ACGTTCCAGACCTTCCATAATAGTACTTAGTCCTGCAGGTTTAAGATGTTTTTCATGAAA
ATAAAACATCTGATAAAAACCTTTCCATGTAGTTGTACGACCAATCCAACGGTTGATATT
CATGTTAATTTCAGAAACATCTTTCCAGTATGTTGATCGAACCTTTGCAATATCCATCGG
AGGCTGAAAGTTATATACGGTCGGTGCTTCTTCAATATCATCAAACAGAGAAACTGTTTC
AGGATACCATTCTTTCATCAGAACTTTATTAAAATCGCCATTATCAGAATGACTAAAAAT
AACATCAGCTCGACGAACAGTTTCTTCTAATCCCAAATTTCGACGCAAAGAAAGAGAAGA
ATGGTCATGTTGATAAACTACAACACGAACAGAAGGTTTAATGTTATCAATAATTTTTTT
ATAGTTATTAATAGTGTCTTCTTCAACTGAAGTAGCAGGAACTGAATTGATAATTAGAAT
ATCACAATCATTTACCAGCTTAAGTGTTTTATCATATTCTTTTGCCAATAAAACCGGAAT
TGAAAATGATTTATAATCATGCGCGCAATTACGAGTAAATGATTTATCTTTAGCATAAAC
CAAAGTTACTTCATGACCATTTTTAATAAACCAATCACGTTGCTCAAGAGAAAATTTAGT
TACACCGCAACCTTCAAGACCTCGAGCCATAAAAATACATACTTTCATTTAATATCCTCA
TTGTTTTGGTTTATTTTACCAAAAATTTATAAAGCAATATAGGAGCCGAAGCTCCTATCC
GCATAATACGCCATACAAAGGCTCGTTAGAGCTTCTAAATTTTATACGCTTATATGTTAT
AGTTCCTTCTGCTTTAGCTTTATCATGAGACTCTTTAAATCGTCTCATCATTTCCTCTTT
AGAGGAACGAATTTTATTATCTATTTCACAAGTCTGGGCGTTCATCTTTCACAGCTGCCA
CCATTTTTTGACTTGATAGGAATCAACCCACACTTTCATATTAGGGTCTGCTCTTACAAT
AGGAGGATTAACTTCTTTAATTGAACTATCATGGTATTCCTTTTCAGAAATTTTCACGCA
AAGATGACCATTCAAAGATTCAGTAAATCCTGCATTAATTTTAAGACGCTTGAATTTTAC
GCGTGTAAGCATCAATCATATCCTCAATCTGCGATCTAGTAGTCTTCCAAAGAATACTAA
TGAGTTCATCGTTATATGGCTGTTTTAGAATATCCCGACTTTTCTTGATAGCATATTCGT
ATTGAGCAAAATTATTGTTTTCAGCTGCAATTTGAGCGTGTTTATAAAGACGGTTCAGTT
CGCGTTTGTTTTTAGACAATAACTTATTTGCTTTTCTTTCTGCTTCAAGACGGTTCTTTT
CTTCAATAGAAGAAATAAGCTTTTCCACTTCATCATTAATTTCGGGTTTATCAGTCATAT
TATTTCTCTAATATAAAATAAAAATCATCATCTGTTAAATGATACCGATAGTTTAATTCT
ACACCATTAGATTTAAAAGCGGTATCATACGGATTTTCTGGATCAATATCGATGTCAAGA
GCTAAAACTTCCCTGAGATACATTTTAAGTAAATAGGGAATAGCTTCAACTTCAGGTATT
TCTTCCAAGAATCCGGAGAGGTTAATCGTTAGCCTCATATAAAAAATCCAAACTAGGAGA
ATCGTCTACAACACTTTTCTTTTCAGCCCCTGGTGTTCTATAGGTTGATTCTTCGTAATG
CGTCATTTTATCGTAGATGTCTTGAATAAAAGTTTCATCTACTAACGCAACCATATCGTC
GTCACGACTGTCATAGACATTGTGAACGAAGTAACTATATTTCTTTGCAACTTCCTTACG
TTCTTTTTTAATACGTTGGACGAATGCATTAAAACAAGCTTGAGTTATATATGCATGTGG
GTTTTTATATTTCGTTTCATCAAAATTATGAAGTCCCTTAATAGAAGCTTCTATACCATC
TGCAATCATTTCTTGTTTCCAAGACTGGGTGTATCCTGAAAAGTTGAAACGTTTAGATAA
GCCTTCTGCAATAAGCATAATGGCTAATCCGATAGTATCATTCTGACGAACTACTTTATT
TGGGTCTTTATTATTTGCTAATTCTGTTTTCCAATCAATAATAGCTTGTAAAAGCTCTTT
ATTGTTTACGTAATTATATTTAGGCTTAGTTTCTGACATTTTCACCTCTTAGCTCAATTC
ATAGATCTATTATATCATAATATTTGAAGATCTATCTTAAAGCATAGAGGATATCAGTTA
TCTAAGTAAGCAGTATGCTTGGAATACTTTCTCCATCATCTTTTTAAATTCATGAATATC
TATATAATGACTAGTTAAATCAGCATGATATAAAAAGTCTAAAAACTTTTCATGTGTGCT
AATGTATTCACTTTCATTTAATTCAGAAACATGACTTTCCCAATATCCAACACCAGTCTT
ATTTGGGAAAAATGAAATTTTCATGTATTGGTCAACATAGAAAATTCCATAATTTTCAAT
AGTAATAGAAATATTAATAAATTTCATACATTCATCAACTTAGCCGCTTCAAGAGCTGCA
TCTAGTGAATCAAATTGGTCAACGTATTCAATCAATTCGCCGTAATTAGCGTATAACCAC
CATTGGCTAAATTCATACTCAAGGATGAATCCGTTTCCTTCAATTTGGGTTAAACCAATG
CCATTTGTATTTACTTCATATCCGGCAAGACGTAAATCGTTAATAAGAGCTTCGTTCATA
TTCATACCTTAGCAATTTTCAGATCAGCAAATTTTTTCTTGCGTTGATTTTTCATACGAC
GAATAGTTTTATCGGAAATTTCATGTTTTTGATAAGCTTTAGATTCTACACCAAAAGCTT
TAACATCAAATTCTGACAAGATATATTGAACCAACAATTCACGGACAGTATTGCGCCCAA
TTTTCTGATCATTCCGTTTCATCGTCTGATGAAGCTCTTTTTCCCATTTATCCAAAATTT
GAGGAGTTACAATATCGCCTTTTTCTAGCAGAGAAACTACTTTATCATATGCATAAGAAT
TGATGGCGTTTTTAATTTGAATAGTCATACATTATCCTCAATTACGTTAAAATTTTATTA
TCCAAAAAGGGCCGAAGCCCTTAGCTAAACTGTTTGGCGCCCTTCCAGCCTTCGTACATC
ATTGCAGCTGATATTAATATTGCTCCTGCGCATTTTGAATATTTGTGATTCCAAAACCAG
TTCAGAAAAACTTCGTCATCAATACCTTGGGCTATCATATTTTCAAAGAACTGACGACGA
GCTTCGGCAATTCGTTGCGATAATTTAGATTCAGGATTCATTTAAATTTTCCAGTTACCG
TTTTCATCAATAAATTTAATCCAGTCATTTACTGACCACTTGGTCGTATCGCCTTTTGGA
GCAACATTTAAAGTGTATAGTCCTTGCTTAAAAAGCATTCGTTTGATATTCATATTTTCC
TCAGCTATAACGATAACACTCGTTTGATTTACGTTTAGCAACTCGTTGAGAAATATTATA
ATCAAAATCATCGTCAATGTAAACTGATTTTTTCAACTTTCTTACTTCACCGCGTAATTG
ACGATTTAACTCATCTTTAACTTCTGAATCAATACCTTTCATTCTACGCCATTTATCTGA
GCGAAAAATGTTTTCAACCATATCTTTATGACTTACCCCATCAGGAGCTTTACGCTTTCC
GAAATAGTCATAATCACGCACTTTTAAGTCTTTACGACGATACGTTTTACCCATGGAGTT
TAATTTCCTTAGCAACTGAACTAAATACAGCACGGTCACAAATCATGTGTTTATGTAACT
TAAGTATAAGATAATAAACATCATAACCATTTACGTAAGTAACGCGAACAGCATTACCAT
TAATAAGATAATACTGTCCCTTTTTAATTTCTTTATCAATCATAACCATATCAATTCCTC
AAAGGCAATTCATGTGTTAATAATACCACGGTTTGAGCTCGTTGTAAACAACTTTGTGAA
AAATATTTTAGGGAATGACAAGAAGGGAACGATAGCTTAGAATGGTAATATACAGAATGT
GAGAAAGAAAGGCCCAGAGGGCCCGTCTTAGTCTTCTATGATATCTCTATCATATCCAAG
TGAAATGAGAGTTTCTTTGAAGTGTTTAATGTTCTTTTGTCTAGAATCATTGATGAAAAT
GACTGGATAACGAATGTTAAAAGATGTGAATCCAGCGCGTTTAGCAAGAGATACAATCAG
TGGACGATCATACTCAATCTTACCATTATTTGTAAGAACTTTATAGAAAGTAAAAGGAGC
ATTGAGCTCCTTTAGAAGTTTTGTAACTGATTGACATCCAGGACAACGACCTACTTCATC
TGGAATTCCATAGACTTCAATCTTGTTTTGTTTCACAACTATTCCTTACTGAGGGCAGCA
TTCAGCTTGTTAGTAATTTTATCCAGACGCTCATTAAACTCACTAGAAGATAAGCCCTTT
TCTGGAGAAATCAAACTAATTACGAAAAATATAGCAATAAAAGGAATAAGGAAAATAGCT
CCAACTGCCATAAACAGAAAGAATGTTACAGTTGTAAGAAAATCAGCTAAACCTTTACGA
AATTTATACATATTTTACCCTTAATTGATTAACCAAGCATTGATAAGCACTAAACTATAT
TGTGAATAAAATTCTGGACCAAAATGAAAAATCATATCATTTATAGTGTCCATAATATAA
TTCAATTTAATCATGTTTCCACACCCCATCGGTATTTGACCAAAGTCGCTGATTATCTGA
TCCTCGCCACAGCTTTTTGGTCGGAAGATTTTTCTCATACTTCCCATCAATAATAACATC
AACATATTTAAGCATTTCTAGTTGTTTAATATCTTCAAACTTATATCCTGTCCACAACCA
AATGCTTTTATTGGGATAAAGATTTTTAATAGTTTGAACCACAGAGTGAATAACATCTCG
GTTATCGGGATAGAGAGGGTCTCCCCCAGTTATAGTTAATCCTTCTATATAATCATTATT
CAAACATTCAATTAATTGTTCTAGTGTTTCACCAGTGAATGGAACGCCATTTCTAGCATT
CCATGTTGATTTATTATAACACCCTTCGCATTTATGCAAACAACCTGTAACGAAAAGAAC
GACCCTGCAACCAGGGCCATTCACAAAATCGCAAGGATAAAATCTATCATAATTCATTGG
TGTTTAACCCTATGCATGATTTCTTTATTTTTACCGAGATTAAATCCGCGTTCGTTCGGA
TTTCCCAAATAACCACATGTTCTTCTTATTGTGTTCATCTTTTTAGGATCAGTTTCTCCA
CAAATAGAACAAACAAATCCGTTTTCAGTAGGAGTCATTTCATGGGTACTTCCGCATGTA
AAACACTTATCTACTGGCATATTAACACCAAAATAATCTAAATGTTGTGCAGCATAATCC
CAGACAGCCTCAAGACCTTTTAGGTTATTTTTCATATCAGGAAGTTCAACATAAGAAATG
TGACCACCTGTCGCAATGAAATGATATGGGGCTTCGCGAGAAATCTTTTCAAACGGAGTA
ATATTTTCTTCTACTGAAACATGGAAACTGTTAGTGTACCATCCTTTATCGGTAACATCT
TTTACACTTCCATATTTTTCTGTATCAAGTTTACAGAAGCGATAACAAAGGTTTTCAGCA
GGAGTCGAATACAAACTAAAAGCAAACCCAGTTCTTTCAGCCCACTGTTTAAGACGAGCA
TTCATTTTAGTTAAAATTTCTTGTCCAATATCACGACCGACAAGAATATTCAATTCATGA
ATACCAATGTATCCTAAAGACACTGAACTTCTACCGTTTTTAAATAACTCAATTATGTCG
TCATCAGGTTTAAGACGAACCCCGAATGCACCTTCTTGGTAAAGAATAGGAGCAACAGTC
GCTTTAACTCCTTTTAAGGAACTAATTCTACACATCAAAGCTTCAAAACATAAATCCATT
CGCTCATTGAACAATTCAGTAAATTTCTGTTCATTGAACTGTGTTCCAATATAAGAATCC
AACGCAATACGAGGAAGATTCAGTGTTACAACACCAAGATTATTACGTCCATCAAGAATT
TCATTACCAGTCGAATCTTTCCATACACTCAAGAAACTACGGCATCCCATCGGAGAAACT
GGAACTGAAGAACCAGTAATAGCTTTATTATTCTTAACCGAAATAATGTCAGGATACATT
CTTTTGCTTGCACATTCTAACGCAAGTTGTTTAATATCATAGTTCGGATCATCTTTATAA
AGATTAACACCTTCTTCAACGAACATAACAAGTTTAGGGAAAATAGGAGTTATCCCGTCA
CGACCAAGACCTTTAATACGATTTTTCAGAATTGCTTTCTGAATCATTCGTTCAGTCCAG
TCAGTTCCCGTACCAAATGTAATTGTTACAAAAGGTGTTTGTCCGTTTGAACTAAAGAGA
GTGTTTACTTCATATTCATAAGCTTGAAATGCATCGTATACGTCTTTTTCTGTTTTAGAT
TGAGCATAATTCAACGCATCGGCGATTTGCCATTTTTCTGCATCCTCAATATGTTTTGCA
TAGGTGCGTTTAACATAAGGAGAAAGTACTTTATCTACATTAGCAAAAGTCGTTCCGCCG
TATTGATGGGAAGCAACCTGTGCAGTAATTTGTGCCATAATTGCAGTAGCAACGCCAATT
GATTTAGGAGTTTCAATCTGCGCATTACCAAGCTTAAATCCGTTCTCAAGCATTCCTTTT
AAATCTACTAAACAACAATTAGTAAATGGAAGAGCAGGGGAATAATCAATATCATGGAAA
TGAATAATTCCGCTTTCATGCGCTTTCATAATAAAAGACGGGACCATATTTTTGGCAATG
TGTTTAGACACAATACCAGCCATAAGGTCCCGTTGAGTTGGAAAAACACGAGAATCTTTA
TTAGCATTCTCGTTTAAAAGGTCTTTATTAGTTTTATGAATCAATCCTTCAATTTCTTTT
TCAATTGTCATTTTAAACTCTTTCTAAGCTGCTTCTTAAATGAAGCTATTAACTGTGTTT
TGGTGTCAGATTCATTATATTCAAATCCTCTTTGAAGCATCTCGGCCATCATTTCCTCTT
TTCCTAAACGAGAAAATTCCTTTGATTTATCTCCAACAAAGTTAGGGTGAATATTATTTT
GGGTGTAATCGGATTTTAAATAAGTAAGTAAATTTTCTAACCATTCAAGATAATCAACAC
CTTGTCCCTTTAAGCCAGAACGATTAAATTTATGTTTCATTTGACCTTCTGCGGCATTGC
AGAGATTACAAAGCAATCCACGTACCTTTCCTGCTTTTGGTCCATTTAATTCATGGTCAT
GGTCGAGGTGATTAGCTTGAACATCAGGATTTAGTTCTCGTTGGCAAATTAAGCATTTAC
CGTTTTGTGCATTATAAAATTTTTGTTTTTCTTCTTTATATAATTTGCCAGTCAATAACA
TAATAAACCCTTACCTTAAATAGATAAGGGTATTTATTATTTTCAAGTATTGTAAAACAT
TTGATTCAATCGCTTATATTGCCGAATCTTTTGGTCAGAAAAAGAAATTTGGGTTTCAAG
CCATTCAATATATTCTGCCGCAGCTTGCATCAAATTTCCTTCATAGCCATCGTTATTTTC
TTGTGCAGCTAATTTAGCTAATGCGTATGAAATACGTTCACCTTGAAAATCAGCTTTAGG
CTTCTGAACAACTTGATTAGTTCTCTCTACAACTTCTTCAATTTCGCCATTTTCTACTGA
TTCAGTATTCCACAAGCACCAATACGTAATTGGCTTATCGTAGATGTTAATAATCTTTCC
ATCAGAAAGTTCAATTTCAATAATTCCAGTATCAGGCTCTATATCATCTTCACATTCTTT
TGCAAGTTCACGAACTTTAAAGACAGTACCTGCACTAAGTTCCGGCCAGTAATTACACAG
CCCTGTATCAGCACGATTAATTCTAAACCACTTATCTACTGTAATCATGCCCCATCTCCA
TATCAATTAAGTCATTTATCGTTGGTTCATTATATACTGTTTCTTCATCAGTGTAAACCG
GTTCTTCCGGCTCTGGCTCTACAGTTTCCCATCTAGCCGCCCACCAAGGTTTTATAGCGT
AATCCATTCTCGTACTGTCTGAGTTACTACACGTTCATGGGGCTCGACTAGTTCAACAGT
CGGAACTGCATAATACCAGTCAGAATGATAAGAACCTGACCGAGATTCATTTACCGCTAC
ATGTACATTATGTTTTGGACTAAAGTAAACACACTGACGATATTGGCATTTACCATCTTG
CACCCAGTCTTCTATTTCGACAGGTTTAAGATAATCCGAAAAATCGAAATCATAATTTTC
TGAAAATGCATCATGGTCTTCGATGATTTCACTCAGAACTGCATCAACATCTAATTTATT
TTCAATAGTCATTTTTTCACCAATTAATTTCGCAATTTATTAAGTTCTTCATAATCAATC
CAGGATTCTAATCCTGATGGCAAATCAGATTCAATTTCCCAAAGAAGTTCTTCAATTTTA
TGAAGACGATCTAGTTCTTCGGCTGGAATAGTAACCATTGACGGAGCTCGTGATACATTA
ATATCGTAAATCATATTTACCCCAATTTAACCATACAATCGCCGTATTTCCACTTAGAAA
TAGACTTTTCACCATTAGAATAATAAACTTCAAGTTTAGCACGATTATTTTTAATTTGAA
TAACCTTTGCTGTCATCAAAGTTCCATACCCATAATAAACTGCAACTTCATCACCTACAT
ATACCGCGCTTCCGCGGTAATCATGAATATAATCAGTTCCTTCGAGAATCATTTAAAGTA
TTCCCGCAATTGGTCAAATCCACCAATATGAATTCCATCAGGAGCAAATACCTGAGGCAT
CGTCAATCCGATTTGAGTATCACGACCTAGTTTAGTCAGAAGCTCAGCGATTTTCTCATC
ATCAAAAACACCTTTTTCCGGCATAATGTTGATAAATTCAAACGGCTGTTTCTTCACAGT
CAAAAGACGTTTTGCATTATCGCAATACACACATTTGTGGATGTTGCTATCATAACCATA
TACTTTAAACATATTATTCCTTAATTCCTAGTACTTGTTTAAAAGTCTCGTCGTAATCAA
GACTTTGACCCGTTTGTTCTTTATGTTTGTATATAATATCACTTACTTCTGATAGCATAT
TTTTATATGAACGAGTTAAAGCAGATTTAAGCACGCTGTATCTATCAGGAAATTTACCGT
GTTCATTATAATAAGCTATTGCTAATTCACGGACTGCCTTTTCAGCAGTCTCCATATATT
CTTTACGCTTGGTCATTTTTAATCCAAAATATTATTAGGCATTGGTTGAACACTCACAAG
TTCTTTAAAGAACTCAGTAGAGTCAGGCAATTTAACCATTTCCGGAGCATGCTTACTGAA
TTTATGTTTACATTGACGGCATTTATATTCAAGCTCTGGTTTACGCCAGTTAATCAATTG
AACTTGTTCAGTACCGCATTCAGGGCAATTAGGAACGTTTTTAGAAGCTCTTTCACGTCG
TTCAACCATAGCCATAATAGCGTCCCAGGCCGGCGGTTTAAAATCTAAATCGTCGCAGCC
GTGAATCTTTCCACGCATTTCCATATCATCTTCCTCACCAGCCATAATAATTTTAATTAA
ACTGGAATTACTTGAAGCTGCAATGTCTTCTAATAAACGCTTTTTCATTTCAGTACCTCA
ATAGCATCACGTAAACCATTTGCTTTTGCATTAAGAGCTTTTAACAATTTGGTGTGTTCG
GCAATCTGGGCTTCAACTTCAATCAAACGAGCATTGAGATATTCGCGTTCTTCACTCAGA
TTATCAACCTTTTCTACTTTAGGTTCTTTTACAACTGGCTTATATGTATTAGGGTTCTTC
AGTTTAATTAGAATGCGTGAACTGTACAGCATTGAACCATTATCTTCGGTTTCGTCATAC
ATTTCTACTACGTCCATCATCTCCACCATCGTGCAGAGTTTATAAGACAACGAACGAATA
ATTTTGTTGTGCTTAGTAATTTCTCTAGCTGTCATGGTGCCGAGATTACGTGAGTAATAA
TAAGGAACCTGGATATTAATTAATACTGATGAACCACCTGGCTTGTGTGTTACTTCATCA
GCAATAACCCAATCCTTAACATGAACTGTATTTGTACGGTCATCGCCCGCGGTAAATAGT
TTACGAGCATGCTTAAAAAGCATATTCACTTGGTCATTGAATTGACAAGCGAATTTATCA
GCAAATTTAGTTTCGGGAATTGTAGATAACCAATCAGCCAAATGGGTCTTTTTAACATCG
TTAACAATAGTAAATTCAACCAAATCGGTTATAGATGTCTCAGATTTAATGAAGCTCGGC
TTCATAAAGTTTTTAATGTTATCACTAGCTACAGTAGAAGATAACAGACGAGAACGTTTA
AACCATTGAAGCAAATTGCCTAACGAATGAGAATAAGCGTTTTGAGTATCTTTACAGATA
TGATTTTCCTGGCCAACATATTGAAGGAAATCACGAAGAATATTATTGATTACTGCTCTG
TGTCCTATCTGATAATTACGGTGTTTAGTAATAAGACTGTCAAAATAATCAATATAATGT
TTACGGGTTTTCATGTTCTTCTCACTTGGTTAATGATTTGTACTCCGAGCCATCCTTGGC
TTTAAATTACTTAATTAACTGTAAAGCTTGTTCAAGACGATCCAAGCGATTAACAGATTC
TTCCCAAATCCTTTTAGCCTGCTCATATTCTTTCTGCGCTTTATTAGAAATTTCTAGAAC
TTCTTTATAAGCTTTTTCTAATGCAATAACTTCTGGACGAATTTCTGCCGGTTCAGGTGA
ATCGTCAAGATTATCCATTAGTTCCTCAAGGGTAATTTCTTCTTTAGGAGTATTCACAAT
TTCATCACATTTTTGCTGGTAAATTTCTTTATCAGTCGGTGAGCACGCACACTTTACTTC
ACGAAGACTAATTACAATTTTATATCCTTCCCATCCGCATATATTCTTAAAAGGATATGT
ATGAATATTTTCTACTGTTTCCATACAAAGTAATGCAGTCTCTAACTGATGAGTTATAGT
GCTAGCAATTGAGAAAAATTTATTAATATTCTTTTGGTTAGCGTTTGGTGTAAAAAATCC
ATAATTCACAAAAATAGCTACTTTATTTTCATCAAGCTCATATTCTTTTACGATAATCAT
ATCAGAAGCCAAAGGATGAATTTGACGATAATCGCCATAACATAATTTAGAAGCTGATTT
TAGAATTTGTCCTTTGAAAAGTCTGAAATTACTAATCCAACGACGAGTAAAAATATTCTC
AGGGTCTTCTTTATTATTAAGATGATAAGAATTAACATCACCGAACCAATTATATCCTAC
TACATCTTTGGTTCGTTTAATTTCTTTCCCAAAATTACCAAGAATATCCCGATTAACCAA
ATATGAAAGAAGACGAGACTCTTTAAAAGTTTTTTTGATAACATCTTTATCTACACGGCT
AGAAATTTTACGAATTTCATGAATAAATTTAGTAGAAGAAACAATACTTGCATCAACACT
ATTGAGCCATTGATTGATAATAGAAAGAACGATATTTTGACCAAACATCGGTATAGCTTT
ATCATCAATAACTCTATTGAATGATTTGATATATTCGTTACGAGTCATATTAATCTCCTC
AGTAGAAAGTAAGAACATTATACCACATCCTTGTGGCAAAGTAAACTAGTTCAGTGCATT
TAGTGCATTGTTCAGTTTAGAACGTTGCTTTGTCAGATTTTGAACCCTTGACTGAGCTTG
TTCTAACGCCTTTTCAGCTTCTAGCACTTCATTGGTCGCTTTAACGAGTTCATCATCTAC
TGCCTTAAGAGACTTCTCAATCGCGTCAGCGTGCCATTTTTCTACAGGTTTAAAATTTGA
ATTTTCAGTAGGAAGAAAACTCACTTTATTAAGTTTACATGCATCTATATTGCTTGAGCT
ATACATCCAAAATTTAGGATCGTTTGATAAGTAATTGGATAAATTTAAATTTAAGTTATC
TTGTACTTCCTGCTTTTTCTCTTGCGTGACTTCGTCCTTTCTCAAAAAATCGTATTTAAA
TGAAACGATCATATCAAGTTCATACGTTGTTGTGTCTAACTTAAATCGTTCAAAACGAGG
TAAAATCTTAGATTGAACAGCAGCAACAACATCCATATACTTAAATGCTTCTGTCAACTG
TGATTTAAGGCATTCACAAATTGAAAGAGAATTTTTTGTATTAGGTTTAAAGCTAATTCG
TGCAGTTCTATTATTTTCTTTTAATGGTCTTACTTCCATTTGAAGAGTATAACCATCAAA
TTTCAGATTCTTTAAGTTAATATCAGAACCTTTTAATCGACTAGCAGTAGACAAAATTTG
CTTTAATTGTCTACGGAACAGAGCAATAAATCTCCATTCAATACGATAATGATTTGGATT
GAAAAATCCAGATGATAAATCGACCTTACTTTGACCAACGCCTTGAACAAATAGAGGATT
TTTATAATCAATAGTTTTACAGAAATCGGTGAGCTGCTCACGAGCAGTCATTTGTGTAAT
ATATCCCGCTTTACTAAAATGACGTAACCATTCACTGCTATTCATATACTTAAAGTGAAT
ATCACGATTAACTTTATCAGGGTCTAAATTATTTTGGCACAAAAATGTCATAACATCACG
AGTATAGCTGGCATTACGAACCATATCTTCAATTTGAGAACGAGTTTTCATGGTGTTCCT
TAAGATTTAAGTAAATCAACAATTTTAATTAATTTTTCACGCTCAGATTTAGCTTTACTA
CTCAATCCAGATAGTCTGAAAATTTCATCGTCATACTGTTGAATAGAAATATTCAGCTCT
TCAATCTGCTTATTAAAATAATCAATTTGTTCAGAATGTTTTTCGTTACTACGAACTGGT
ACAGGTTTTGTAGGCAATTTAGTTGAACTGGATTCATCCGGGCGATAAATTAAAATACAA
CTTGAACCAATAGGGCATTTTGCACCAGATGAATATTCTAATGTTCCAGCTTCTTTTAAA
ACTTCAAAAGCTAAACAGAGATGATGCCCCATATTAACATAATCTGTAGAGCGAGCTCTG
ATGTAATAATCATCTCCGCGAATACTAAATTTAAAGCATTTAAGGTCTTCTGTATTATTA
GTTTTAAAAATCATTTGTTTATCTAGACCTTTAGCTAATCGAGCGCCTAATGCTAATAAT
CGTCGTTGATTTTCCCACAAATCCGCAATCATTTGATCAAATGATAATTTCGGCATTAGT
CGACCATAAAGGTCGTATCCTTTATTATAATTACGAAGAATTTCACTCGCATTAATACGA
GACAACGCTCCAATTTTATTAAGGGCTTTCATAATGCGATTAGATAAACTCATTCCAGAT
CCGTTTGTTCGCTGCAAATGTTTTTCTAATCCAAATCCAATATCAACTTTAAATTTATCT
AAAATTTCAGCATGAACATCTCTGTCAATAACATTCAAATCCAAAGTTGTGTTAAATCTA
TGAAAAAATTTATCTGGCTCTCCACGACGAAATACAGTCCATTCATTCATCATTTTTTTA
TTAACTAAAGATTTAATTACTGCATTGACATTATTAATTACGACTGACATATTTTCCTCA
CTCAATTTCAATTTTACTAAATACGCAGAATATGATGGAAAAGACTATATAAGCACCACA
CACAGATTGAACTAATACCATTCCAAAGAACCAAACAATATTATCAAACAATGTCTGTTT
TACGTCGAAAGGACGTAAACTTACAGTAATATTATCGCCTTTTTCTATTGATGAATACAT
CTCTGGTGAAATATATTCGCTAAATCTATAACCGTCATTGAGTTCATATACAGCAATAAA
CGATAAACTAGACCCCTTTCCTTGAGTTCCTGTAAGGGTATTAACTACAGTAACATCATA
ATCTTTATAATGCATATAATCATTAATTGCGTAATAACCATATGCAATTACTATACATAA
ACAACATATCAATAAATTCATTCTTTTAATTATCAACTGTTTCATAATAATCTCAGTTAA
AAGGGCTTAGAACCATTATACCATCCTTGGTATAAAGCGGTTATGCGAGTACCGTCTTTA
ACCGTTCTTCAAACTTCCGAAGAGTATTCTGGCGTTCAGCTCTTTGCTTTTTGTAAGTTT
CAATACGCTCTGAAATGAGAGTGTATCGTTCATTTACTGATTCTTTCATAAAATCAGGAA
TTTCACGAGAAGCTTTAATCTCATCAAATTTATCAATAACAGCTTGCTCTTCAGCAATTA
AGTTATCATACATCAAAATATCTTTCTTGATGAACTCAATATCTTCTTGAGTTACACGAG
ATAATTTAGATGCTTTATCCTTTTTGTACTGTTCGTTAGTATCACGAGACCAGTGTAATG
TACGATTTTTATTCGTATTCTTGTAAATTTCTACAATACCGATCTCATCAATAATAACGA
TCCAATTCCAACGGGATTTGTAAATTTGTCCTCCATCAACAGTGATTTCACCTCCGATTG
AGATGTCATTAAAAAACTTGCTTTGTGCTTCAGATTTAAATTTACCATCGTTGTAATTTA
CCAGGTTGAAAATATCTTTAGCGTTCATTTTGTGTTCCTCCGTAGTTGATAGTTGTATAG
TACCACAGAGGAACGGTCTTGTAAACAACTAAAAGAAACTTCTTTCACAATTTTTTCCAC
TGAACCATGCGCTCACTGCTTTCTTAGTCTCAGGAGCAGTGTTATCCATAAACCATTCAA
AGGCAGCCTTTTTATGATTCTGGAGGGCTTCTCGGGCTTTAATCTGCTCACGGTCTATTA
ACACTAACATATGAGCCTTTCTTGTCACCAGGGGCTTCTTATGATTCTTGGAGTATTCCC
AGTCATTTGTCCAACGCATTGTCGTTGCGAACTGAAATACTGCTTCTTTAATTTTAGTTT
CGTAAATTTCACGAGCCTTTGAGTATAACATCATTACCTCCATTTACCAGTTTAATTCTA
GTCATCTTTTTGATGGCAGTCCATATAATCTATTTCTGAACTGCCTTTTTGTCTTACGAT
TCTTCTAAATGTCTTTTCTGAAAAGTTGATACCATACTACGATAAGGTTCATTTGGATAC
CCGTGTTTTATAGCTTCCTTTGTAAATTTTCTCCATCCGTATTTAGTTTCTTTCCAAAGC
TCGAATAATTCATCTTCATACTGCCATATAGTTAAATGAGAACGCTTTGCAGCTTTAATT
CCATTAATGTGCTTTTCTTTTACTGTAGGATTGCTTAGTGTCTTTTTAATAATATTACTA
ACTTTTCTAACAAAAGCTGGGTCCTTATATCGTTGTGAAGTTTTTATTGAAGTGTTTAAT
TTAGCTTCCGGCGTGGATGTGACCTCTTTCATCTTTTTCTTCATATGCTCTATATGATTA
GGATTTTTCCATAAATTTATAGAATGCTTTTTACATTTTATCCTAGTTTCATCTGCAACC
TTTCCTCCTTCACCCCCATATGACAGGTTAACACAGTTTTTTCCATATTTCAATTTACCT
GCCATGATATATAATATTTCGGCTTGTACCATTTTATCATAATTATCAAAAACTTCTAAA
ACCGTTTTTGATAATATATGGTCATTTGACACTTTTACACGTTCTATATAATTTCCACTA
CCAACATACGAATCATTGATGTTATTAGTTATATGTCGACCATAATAGTATTTCTTAATA
CTTGTCTTTAAATTTAATATTTCAGTTTTATAGATAGTAAAATATTTTTCATTTTAATAA
TACCGTTTCAGCAATTTCTTCCCAATGGTTTTTGTCGGTCATAATAAAGTCAGCAAGATA
AAGAGCAGTACGCAGTGAAACATTGCGTAAACGATTAACATTGACTTTCATCCATGATAA
TGCTTTATAAGTTTCTTCATCAGAAAGACCGCGCTTTTGCATCATGTCAGTTGAAAGAAT
AACATCTTCAACCCTGACCATAATTTCTTCATTAGTGTGAACACCCAAATCCAAATAAAC
TGAGCGGGACACTAATGCTTGTAAATGTGGGGCAAGTTTAGTACCACGGTCTAATTCGCG
GTCAATATCAACGTTTGTGATAAAAACAATCGTTCCTTTAAATTCAAACTCACGGTCAAT
GCCTTTTTCTTCTAAGTAAGAAGACGCAGTGCTCCAGCAGACTTTACGGGTTTCTCCAGT
GTCCAGAGCAGCTTTCAGAAGATTAAGAATGTCCATATCAGAGAAAACATCCACATCATC
AATCAAAAGGACAGAATTCTCTTCACGATTATTCCAAAGCTGCTCATAAAGACCGATACC
GGAGATTTTACCGTTAATGCTTTTATATTCAATGTATCCATTATCATTTGCTTTATTTAA
AGCTTTATCTAAAGAATACGTTTTACCAATACCCGCCGCACCAGAGATAATTAATGAACG
AATGTTTCCGTTAATAATACCATTCGTCATCATTCCCATAACATTAAATCTTTTATTAAT
GCGGGTTTTCATATCTTCATATGATTCTTTAACTTCTTCAACTTTTACACCATCATATGA
AATATCTGATTTGTAAACCCAAACACCGCGACGTTTACCGTCAATTTCAACAAAAACTTT
ACCATCTCCTTGTGCATCTACCGGAGCATTATCTGGGAACCATTCGCCTAAGAGCTCAAA
AGTTCCAGAGATTTCTTTACCGAAGTAGATACCCTTATTGATAGTTACAGTTTTCATTTT
ATTCTCCAAATCCGTATCAGTTGATAGTTGTATAGTACCATAAAGCTTTATGCTTGTAAA
CAGTTTTGTGAAAAAATTTTTGAAAATAAAAAAGGGGACCTCTAGGGCCCCCTTTGTGTT
GATTTATTCCATTTCTACTATATGCATGTTCTTTTTGCTATCAAAATATATGTTTTTAAC
TATTAACTCAGAATTAATTGGCAGTAAAAACTCTCTTTCATAATCTACCATACAAAGATT
ATCATAAAGCTTGTTAAGCTTATCATCGTCAGTTTGGCCAGATTCTTCAATTGCGATTAA
ATTTTCTAAAATGTCAATCATAGCAGTCGAATAGTCAAAAACGTTTGCATTTTTTAACAC
TACTAAAACATTAGTCATATAGCGTTCTGCAGCAAACGTTTCAGCTATATGAGACTCACT
GCTAAAGGAAGTTATTCTTTTAAATTTGATTCTCCTATTAACTTCAAACTTATAAGATAG
CACTTCCTCTATTGTAAGTCCTCTAAAAAGTATGTCTTTAATCTCAAGTTTGTCTTTAAT
GTAATCATTAACACGCTTATGATTAAAATCATTAATTTTGTTGCCAAGGCATTGCATTAA
AATATGCATTGTATTCTCCTCAATTTTTAAAGAGCAATTAATTAACTACACATATATTAT
AATATGCTTTGCGACAGAAGTAAACAACTTTGTGAAATTATTTTAAACAAAAAAAAGGGA
GCCCGAAGGCTCCCTATCATTTATAATAACTTCGGTGGTTTTCAAGATAAACCCTCTCAA
GGAAGTCATCCCAGAAACCCATGTCTACTTTTTGCTGCATACCGTTCTTAGAAGCTTCAA
TGGATGCTGCTTCTACTTGATCGACCACATCTTCCAAAAACTCTTGAACAGTTTTAAATG
GATGCTTACCCAACTTCACGTCGAGAATAAACGGAGCATCTTGGAGTGGATAGACTAAGT
CGCCAGTTTTGTAAATTTCCAATAGTTGAAGTCCACCACGACAAGCATGACTCAGAGCTT
TCCAGTCAATACCTTCATTGGCTTCGGCCTTACGAGCACGTTCACCATATTCAGCATCTA
ATTTGTTCAGTGACTGCTTAAGCTCAATAAGAGAAAGCGTTGTCTGATATTTACGACCCA
ACACAGTGTAGAACGTTTGTGGACCGGTTTTCTCATGATTATGGAATACCCATTCGCAGA
ATTCGTTCTCTGGAAGACGATGCTTAATATCTTCAACTTTAGTGCGACGCTGCTTAATAG
AACCATCTTCTTGGTAATCAACCCATTGCTCAGGGATTTGATTAACTACTTTCAATACAT
CGCGTAATGCAGCCAAACGAGAACCCTTGACACCGTATTTAGAAGCTTGCTTGCGGACAT
ATCCTAAATATGATTTCATGTTAGTCGTATAAAAACGAGAACGGTTGTCTTGAATAAACT
TCCACACATCAGGCAAATCAGATTTAACCACTAGTTCAGGTGGAGTGTGAAGCATATCCA
ATGCTACAGTTTCACCATCTGCTGCTAATTTAAAGAAATACTTAAGACTGTATAATTCAT
GGTCAATATCATCTTTAGTGTTTTTAGATGATGTATTGTTAGTGTTTTTACTCATATGCT
CTTTAACATTTCCGATAAGAATATCACGAGCAGGAGGAACAAAGATTTCTTTAAAATCTA
CATCAGATTCTGGGGTAGAAGTTCCATAAAGATGACTACCAAAATAGCTTTTCATTACTG
TTTTCATCATTCAGCCTTATATTCAATAACAGGACATACTTTAGCTTTACGAGCTTTTAA
AAATTCAACGATAATCGATTTCTTGGGTTGAATAGGAGGTAAACCTTTATACGCTCTATC
AATATTTTCTACGTGTAAAGCATAATCCTTTTTCCATTTATAATCTTTATATTTTTCACG
CAAATGTGCAGGAATAACAAATGAACCGATAATAAGTAAAACAACCAAAAGCGCAAGTAT
AAATGCTGATGGAAGAAGAGTCCATAAACTACCGAAAATAAGCCAACGTTGTAAACTTTC
TGTACAAATAATTGTTAACCCTACGAGTATAGCACACCCAAAAATAGAAACGAAAAATGT
AGGAATGGCAATAGACCAAAAATATGCGCATAGTGTCTTAGGTCGTTGCCATTCATCGTT
AAACAATTTGAATAATTTATAGTGCCAAGAGTTTTCATTAATAATCATAATTATCCTTTC
ATTGAAGGCGTAACGGTTGTTAAATACTTAATCATAGCTTCAGCTTCTGATTTTGATAAT
GAAATTTCTCCATTGTTTTGACGAATAGAAATAAAATCCGGATGACGGTCACCACCAGCT
TTTAAAACACACATATAAGTATCAGTTTCATCTTCAATATTTGAAATGATTGAAGCAGTT
CCACATGAATGTGGTTTTTGATAAATTAATGCAGCTTTTCTTCCGCCATTTGACTGTTCA
TAAACTTTTAATACATCAAGCAAATCAGTTCTTTTATCAATAGCTAAAATGTTGTTTTCA
TCTTCAATGTTAAATCCTTCGGATACTTCATAAAGCGTTACCCATAAATCACCTATATCG
ATAGTAGCATTTTCCTTTCCATGACTTTCAAGAGCATGACAAATTTTCTTGATTTCATCA
TAACTCATTTTAATAATATCATCTCCTTGAATAAGGAAGATGTCATTTTTAAATTCAACA
CGAATTAAACCACTTCCACTTGAAATAAACATATTTCCTCACTTTGAAATCATAGTTGGA
ATGACAGAATCAAGATAAGTCTTTAGCGCAATAGCTTCGTCTTTTGTAAATGTGACAATA
TGTGATCGAAAATCGTCAATTTGGCGAATAGTTAAGACATCTCCATCTTCATAGCATTTT
GATACGTTTAAAACAGTTTCATCATTCTGATTGCAAGCGTTAGTAATAATAGCATTGCAA
TTTTTAAGCCATTTTAAACTGTGTTTTCTTTCATTAGAATCATAAAAATATTTAATGTAA
GTTATAAAATTATCCCAATTATTAATTGATAAGCACATTGACTCACTTTTAATATTAAAT
CCTGGGCATGAAGAATAAAAATGAATTTTGTGCTCATCATTGATGCTTACTATTTTATCA
GTATAAGGATATTCAATTTGGGTTAAATGAATGATTTCACTAGGCGTAAAATATAGCATA
TCGTCTTCTTGCGTCAAACGATACATGTTATTTACTTTTTCTATAGCCAATTCACCAAAA
AGCGGACTAACTTCTAATACTAATCGTTTCATATTAATCCTTTTTATTTGCGCTGATTTG
TTCTAAAATTTTAGAGTATTCCTTTGTATCAGATTTACGTACAATCGATACACTATACGG
CATACTATCGGTGTCTTTTATAAAGCGTTCCAAATGACACTCTAATAAAATTTCTGGATT
ATAAATCGTGTACATCAAATTACCTGTTGCTCCACGATATTGACAGATTTCTACTGATTG
AACATCATTCCATAATTTTGTTAAGATCAAATCACCTACTTCTAATTTAGGAAAGCCCAT
GTTATTCACCAATTAAAGTTTCAATATAAGAATCAAGATTTACTTTAGCTTCTTCCATAA
GAGTTTTGTATTTAATCTTGTTGGCATGTCCGGAGAAAGCTGATTCATCAATAATTGTTG
ATGTATGAACAGCTTTACTATACGTCTCAATTAATTCTCTTAATTTAAAAGCCTGCTCAC
TTGTCATTACATACTCCTTTGAATAATATCAATAATGTTGTTCACCAGATTATAAGTAAA
CATTGGGTAATTATATTGAATCATCACATACACAACAAACAAAACTTTCATTCTCTTCTC
CTTGACAGTTGACAAGATTACTATACCATAACCTTGCCAACTTGTAAACTGTTAAATGAC
GTTTTCAATAAAATTCTGGAGTTTTGTGTGAGCATCAACCATGATTTTCATTTCTTCCTT
TGCGAAAGCTATGCCTCTTTCCCGAGAAGAACACTCATAGTCAGAAACTGCATTAGCATA
TTCTTCAATTAATTTCATTAAAAACATCTGCTTTTCAGTTTTCATTATTCCACCTAATCA
TTTCAAGATATTGAACTAACTTAGCTTTAGATTTATCCAAATCCTTTTTAGCTGCTTCTA
TACCGTCGTATGAATATCCTTCACAATGCTCAACTGCTAATTGATATGAATCTATTTCAA
TATCACGTGCTAATTTAATGATTTTTTCAAACTGTTCGCGTGTTAGCATAATTAAACTCT
CGTATTATGATCGATAATTTCATCAAGAAACATATCTAACGCTTCTAAAGCATTATCCAC
TTTAGTTTCAAAGTCTTTAATGCCTTTATTCGAAATACCCGATGAATACCAAGCAAAGTC
AATCAGCTCTTCATCTGCTTTACGAATTAAAGCTACTAATTCCTTAATTTTATCCGCTTG
TTCGATACTAATCATTATTCCACCACATATGAAAGAGAGAATATTGCACACGCCAAGTGA
GTTGCAACTTCATCACACATATTATAACGTTTCTTAAGAAGTTCTACAAGTTCTTCACTA
GTAACTTCATCCATGTCGACGAAAAAATCACCATTAATGATGACGTAGATATTTCCTTCT
TGATTAAGTGCTTCAATTTTCATGATGTTCTCCTCTTTATCCGATGGTTGTATAGTACCA
CGGCTCAAACGAAAAGTAAACCGGTAAAATGAAAAAAGTCTCCCGAAGGAGACTATTGTT
ATTCGAGGGAAAGAAGATACTTACTCTGGTAAAACATTCCAGTAATATCATCTATCGTGC
TTTGAATGGCTGGAGGCATTTCTTTATAAATGCTGTTAGATTGGTCTAGTATGCGATCAA
TCATTTTAACTGTATCAGTAGGAAGTTTACTAGCATCTGGAATTGAAGGTGTGTATTTTC
TACCAGAATACCCCAAATATTGCTCACCAAATTTATCAATCAAATCTGGCAACTCAGAGA
AAATAAAATCGTATGCTTTGTGTCTAGCATAACTTTTAGTTTCAAAATGTGCAGAATGAA
AATAAGCTTGTGCAGCCATTAATAAACCTAAGTATTCATCTGCCTTTGAAGGTTTTCCAC
TTTGTGAAAAGTCGCTGAATTTCATTCAGTCTCCAATTTAATGTTCATAATTCTAGCGTA
TGATTGTGCCATCTCCGCGCCTCGTTCTATACATTCAAAATCAGAAGAGCACGGGTCATT
TTTATAGGTCGTTCTCATAAAACTATAGAATTGTTCAGACGATTCTACGCTTTTATTTTC
AAAAAGTGTATAAACGTGCCTAATACCAGATTCCATAAATTTATCAAAATGAGGATCGAC
ATTCGCTTCAATCGATGGAGATAAAGCAAATGACAATCCTAGCATGGCAAATAGTGCCGT
TGCTTTTAAGGCCATAAAGGCCTCCTATCATTTTTGTCCTGTATTTACTTTATGCCGATG
CACGGCCTTAACTTTATCAAGGTATTTTTCAAAATTTCGCAATCTAGTATAGTCTGCCGG
AGATTGGTTGAGTGATACTTCTCGACGCAAAGCTGAAATGATATTTCCAACTTCCCTACG
AATTTCATCTAATTGAAGCACAGTAAGATTGCGAAGTTGCTTTTCAGTTAATTGTAGCAT
ATATACCCCTTTAGTTAGATAAACCTATTTATAACTTTTGCACTAACCGAGTTTTTTAGT
TAATTCATTCCAATGTTTTCTACACAAAGAAACATAAATTTCATCACCAATACAGATTTG
ATTACCTTCTTTAACTGGTGTTCCATCTTCCATTAATCGAGCTGTCATAATAGCTTTTTT
ACCACAATGACAAACTGCCTTCAGTTCAATAAGTTTATCTGCAATCGCTAAAAGTTCTTT
AGAACCTTCAAATAATTTTCCAGCGAAATCAGTCCTTAGTCCATAAGCCATAACAGGAAC
ATTATATGTATCGACAATTCGGCTTAATTGATGTACCTGTTCAGTTTTTAAAAACTGAGC
TTCATCTACAAATACGCAATGAATATCTTTTTGTGCTTCAGCCCATTTATAGAACTCAAA
AATATCCATATCATCCGTAATAATATTCGCTTCCTGTTTAATTCCAATGCGAGAAACGAC
TTCACAGACAGAATCGCGAGTATCAATAGCAGGCTTAAGAACTAATACACTCATTCCGCG
TTCTTTATAATTATGCGCAGCAGTCAAAAGAGAAGCAGATTTTCCAGCATTCATTGCTGC
ATAAGTAAAAATTAAACTCGCCATATCACCTTCTTAAAGCGTATTCACATAAGCTATTAA
TTCGTTTTCTTTCTTATCAAACCGATCAGCAAATTCTTCCTGCTGTTCGGGAGATAGCGG
GCCGTATTCATCATAAAAGGTGTTTGCTTCTATATGCTCATCTAGAAGTTCATGAATAAG
CTCAAATAATCTACTTTTCTGACTAATGCTTAGACTCATAACCATTGCACCTTTAAACAA
TATTCTTCTACATGCTGTTTACGTTTATTTTTATCTATAAAAGTATATTCAACATATTCG
CCGATATACCATTTATCGGTGTTGTGTTGACTTTTTATAGGGCATTTAGTTACTCGTGTT
TCGGTCCAGTGTCGAATAATTTCTGCTTTGTTTTTAGGATCAAACGGATGCGGGTAATGT
GCTTTCATAATCCACCACTATTAAATCAATATCAGGAGTAAACATGTCGATTAATTTAGA
AACCTTATCCCAATCACCGCCAGCAATACCACAACCTATGCGTGGAATGTAAATTACTGG
TTTAAACAAAAGGGTTTCTGCCTGTAGATTTAATTCTATCATGCAGTTTACTAAAGCACC
ATAATCAAGATTAGGACCCGGTTCGTATTGCGTATACAGGTTATAGCATATACCAAATCC
CGTAGGACTATGTTTAAAAACAATAGACATATCGCCTAATTTATCACGAGAACCGTACTC
AGTAGTGGTTTTATCTATTTCTAAAATTTTAGGATAGGCTCTTGCTAATTGACCCGCTAC
GCCAGAGCCCATTGTGTGGAAGCAATTGCACCCGTGCGCAATAATATTACCTTGAAGAAA
TAGGGCGACAATATCGCCCTTGATATATTTTACAATCATCTAGTACTCAATCCTCGATTA
TAAGAATCTACCAAACGGTCAACCATTGAATGACAAGCGGCTTTATCTTTCTCCTCCGCA
ACTGAACATTCTAAGGTATTCCACTTTTTAGCATATCGTTTTAATAAAGTATCGTTCTTG
TATCTACTTGTTTTATCTCTTTCTCCGTCTTTATATGCATATATTAATTTCTGCGCAAAT
TCAGCTTGGCATGCTTTATTTTTCCCACAATAATCCGCGGCAGTACGGTTTACATACTCT
CTAATTTCAGTATATGATGTATCTGCTAACGCAGAAAATGAAATTAATCCTATACATAAA
ACCAAAATTTTAGTCATTTACTATTTCCAAAAGTTTATTATTTTTAAGGTAATTAGCCTT
TTCTAGGACTTCAGAAGCATATTTAGAACCAGCTTTAACATTCCATCCCGAATTATAAGA
GGATATTGCTTTTCTTATATCGCCCTTATGTATATTTAACCAATAAGAAAGTTCAATGTA
CGCCCAGGAAGCTGAATTGGATCGTTTATTCAACATTCTTTTTATTTCAGCATCGGTCAT
GTTATAACCAAGTTCCTTAACTCTTGCTCGCATAGTAGGCAAATAATTTTGGAACATTCC
ATAAGCGTGATGCTTTGGTTTAGATTTTAAATTAACTCCGCCAGAGCTTTCTTGCCATAA
AATAGCAGCCATTATATGACCTAATCCGCTCTTGTGGATATTTTTGTGTGTTTTATATTT
TCCATCCTTAGAAAACTGTTCCCCGAATTGATACGCGTAACGCATGTTATCGAGTTGGAC
ATTACTGAAAGTATGCTCGGAGCTATGTGCCATCATTGAAATGGCCAATAGACCAGCGAG
TAGTGCTTTTCTCATGCTTACCTCATTGAGTTTTAATTACTGCTTTAGAAGCCTTTCCTG
GTAAACGACGACTGTTAATAATTGCCATCCTGCATTGAAGCGACGGGTCTTTGAACTTCG
CATTAGGTTTACAAACTGTAAATCCAAGCCAAAGATTTCCATCTGTGATTTCTAAACGTC
CAGGACGATATTCAACTCCATCAATAAAATCCTCGTCAATGTCAGGACGCGGAGGCATAC
TCAGGAATTCATTAACTTCTAAAACATGGTCTTTTATTTTGTGGAATAATTCAAAAACAT
ATGTCTCATCAATCTCCCGTTGAATTGCACGATCAAGAAGATGCTGAGAATATTTTAGAT
GAAACGATGAGACTCCTGCTGCTTTTGATGCCTCACGAATCTCATTGTTAATTTGACGAA
ACTCCGACTCAAAATGACGACGAAGCTTATTTCGACGGATAAAAACTTCTGTGTTGATAG
TCATGTTATTCTCCTCTTAACTGATAGAAAAATTATACCACAGTCAAGAGGAAAAGTAAA
CAGTTATTCTTTAAATCTAATCAATTTATTCATAGACTTTGAAACTTCTGCACGAACCTC
ATGTAGATTTTTGAGCTGTTCAAGGCGCTGCGTATAGTAAGCAATTTCATCTTCTTCGAG
ACAGTCCTGTGAATCTTCTTTAAGATAACGTGCATAGTCCTGAAAAGCGTTACGGACTAC
TTCCTGGAAGTCATCAAGACTTTGAATTTTCTTAGGAGCAACAGATACACGACGAGGGGC
AGTATAATACTCATAACCAAACCCTGCGCTTAATTGAGCCATTAGTATTTTTCCTCTGGT
TGGAACACAGCACGGCAAGCCCACATACTTGCTTCTTTGAGTTTCGTTTTAGCAATAGTT
AACTGATCGAGGCTTTCGGCGTAATTCTTCGCGAGTTCATCATCTTCACAGCTATCAAAT
GCTTCCCAAAATTCATCGTATAAAGCATCAAAGATAAGTCCTAAACGAACTTCAGCGTCT
TTAATAGCATTTACTTTACCGATTTTTTCGTCGGTATGTGGCTTATAACCTTTAATATCT
TCAATCATATTTGACTTCCTCACCAGTACATAAATCATATTCAACTAAACGAATAGGTTC
ATGAATGCCATATCCCTGAACAGAAATTTCTGTCGTAGGATAAATTCCATCAATATCACC
CATATTCCAGGCTTCATTAAATTGTTGTTCGCCTGAATTACTAAACCATTCGGCGAAAGC
ATTTAGCACATCTTCAGAACCTTCAATAATTATCTTTGCCATTACAAACTCTCGGTAAAG
GTACGAGCGATAACGTCGCGCTGCTGTTCCGGAGTCAGAGAGTTAAAGCGAACTGCATAA
CCGGATACACGGATGGTCAGCTGCGGATATTTTTCCGGATGCTTAACTGCATCTTCCAGA
GTTTCACGACGCAGAACGTTAACGTTCAGGTGTTGACCACCTTCAATTTTAACTGTAGGT
TGTGTTTCAATTTCAATTTCACGGGCATGCAAACCGTAGAAAATTTCCGGGTCTACAAAA
GAGTCCTCTTTAAAGGTTTTAGAGACAATAATTCGTGCTTGAATACCATCTTCAAAATAA
ATAGTACCTTTATGTGTGCCTTCAAGAATTTGATATGCTTTCATATAAACCTCAATTAGA
AAATAAATTTATCCAAGATTGTTCTTTAATTAAAAATGGCTCAGAATCATATGCCATTAA
ACTTTGGGTGATTAGTCCTTTAAAAGGTCCATCAATAAATTCCATGGTAAAATATGGAAT
TTTATTCATTAGCCGTGCATTAGGAGCAGTGCACAAAACTCTGCATCCTTTGAATACGCC
TTTTTGTAATTTGTATTGCTTGGGATAAAATTCGCTCAAAATGTTATTATTTGCCAAAAT
TTCAAAATGATTCACCAATTTATTTTTAATAGTTTTTGGCGAAAAATAAAGATATTCGAA
AAGCTGTGTGTCTGTCATCATTGCATTCCAATCACAAAAAATTGAGGACGAACTGTTTCA
CCTGTTTCAGCATTATAGCTTCGACAACAGTGTTGTACCGTTTTAATATGGATATTGTAA
ATCTTATCCATATCAGGAGCTTTAATAGGTTCGTCAATTATGTACAAGATACGAGAAAGC
GATAACCCACGGAATTTATGACCTTCATTACCGATAAAACTACGGACAGAATCAGTAAAT
AAACGAAAACGTATATCATCATTAGAATAACGCGAAAATTCCTTTTTAATGTTATTTGCA
GAAATTTTAGCGTAAGCTGAAGTATTAGAAAGAACAATAACTGTTCCGCCATCATACAGC
CAATTAGCAGCAAAGTTAGTCACAGCAATTGATTTACCGGATTGACGTCCACCATCTATA
CGAAGTGTGCAGTACTGTTTGAGCACATCTTCCATTGGTGGAAGATAATCATTCTTACAA
ATTTCTTCTACTCTAGCATCAGAATGGTGTGTAAAAGCATTCATCAGGGATAGATAAGGA
CCAGTTAAAAATGTTCTCATTTTGTTCTCTCTAGGTTTGGGCCATTCCATGGCGCATGAA
TTGTCCATTTCTGTATTTACCCATTACCGCGCTTGGGCTCGACCTTATTACAGGTTGGCG
GGAATCCCTCAAAGAATCATGAGGTCCAGCTTGTTCCCATATTTCTTATTTGCTTATTTG
AACGATTTAACGTACTGAATAGAACCCTTACTATTCACAAAGGCAATTTTACCATTTTTA
TTATACATTGTAGCAGTACGCTGATAACCATAGGTTTGTTTACCAGATTGATAACCAACT
AATGCAAAATTATCTTCACCCATTTTATTCTGTTTAGCGCGAATGCGTTCATGACCATGT
GCCATAAGAACCTTAGCATCACCAGTTGCAATGGATCGACGAGTTTTTTCATCTTCAACT
GTATTAACAAAGTCGTCAAATGAATCAAATTTAGCGTGAGATTTATTATACAGTTTTCCA
TCTACTGCATAATTGTATCCTTGTACATCCATCTCAACTTCTGTACCATCATCTAATACA
GCAGTAATTTTTTGACCTTTTTTAAATTTCAAAAGATCAGTAAAAGATTCAGCAATAATT
TGAGAATTAATAGCTGCTTCAGTGATAAATTCTTGATATGTTTTCATGGGTTTCCTTTAA
ATGTAAATATTTTTACCGTAATACTTATACCATTGTGGTTTCATTAAAATTTTTTCATCA
AGTCGTTGTTGACTCAATTTAATAGCTGATTTGCATGGATTATAATCATTTTTCCATTCT
ACTGGAATATCGCTAATGTCAGAAACTTCAGTATCTTTTATACTGAATCCGCGTTTTAAG
CATTCCGTTATAATGTCTGATTGACGCTTTCGCAAAAATTCTAACTTATCGTAAAAGAAA
GTAACATGACCAGAACCTAAAATAAATTTAGAAGATATTTTAAAATCTTTAACGCGCTTA
CCGTTTGCCACATGCTTACGAACTATACCAAAAACACGTGGCAATTCACGATATTCTGCA
ATTAAATGCTGATCAGTAAGTTCAGATACTAAAGTCAAATTAATACGAGTCATTTTATCC
CTCCAAGTAGATGTGAATATACTATCACAATTCTAGGAGAAAGTAAACAACTTTATAGAT
TTTTATACGCGTCCCAAGTGCCAGTTCTAAACGTTGCAATGACTCGTTTTGCGCGATTAG
GTGTTTGATTATACCATCTACTTTTAGCTAAGTTAACTGCTGCTTCATCCCAGCGTTTTT
GTTGAAGCATACGTAAAGAATTAGTAAATCCTGCCACACCGGTTTCTCCCATTTGGAAAA
CCATATTAATCAATGCACAGCGACGAACAGCATCAAGAGAATCATAAACTGGTTTTAATT
TAGCATTTCTCAAAATTCCACGAACAGCAGCATCAACATCCTGATTAAAGAGTTTTTCAG
CCTCATCTTTTGTAATTACACCATTGCAATTACGCCCAATAGCTTTATCTAATTCAGATT
TAGCAACATTAAGTGATGGACTTTTTGTAAGCAAATGACCGATGCCAATAGTGTAATAGC
CTTCTGTGTCTTTATAGATTTTAAGTCTAAGACCTTCATCTATACGTAACATTTCAAATA
TATTCATAATACCTCCTAAGTATTTATAGAAGGTATTTATAAAATTAAAAGAGGTTGTTC
ATTATTCGGTAAAGTGAAGGACCCATCACATATTGCCACTGAGTGCGAGGAATAAGAGCA
AAAGCATCCATCTCTGGAATCATAACGCCATCTTTATTTTCAAAATAAGACTCACAATGG
CAATTTCTAAACATCTCATGCTCTACTGGAATCGTATAATAAAATAACTGTAGGTCTTTA
TTACTAGAATATTTAAATACACCTAGGTCTTCTAGAAGGTCTGGATTATAATTGCTAAAG
CCAGTCTCTTCTAAACATTCTCTTCGTGCTGCATCTAGCGCGCTTAAATCAGAATTTTCT
ACACGGCCCTTTGGAATATCCCAGCGATGGGCCATCATTCCAGGCTTACGAGAACCGGTA
ACCCGACCCATAAATAAATCTTTATCTTCTGTCATGAAGATAATACCAGCTGATAATGTT
TTCATTTTAATTTCCTGCAATCACTCACAAACTCTTTCATTGATTCATTTTCAATATAAG
ACATGTAGCTATTATATTCTTTTAATTGTATTTTGTAATCCTTTTTTCTTTGCCAATTTA
TTTTAAAATTATCATAATGAAAATATAACATGGTACCAAAGAATGAAAATAATGAAATAA
TTTTAGTATAGTGAAGTTCACTCCAAACTTCTGTTATATTTACTGTGCCGTCGATGTTTA
AAATAAGACAGTCAATTAACAATCCAAAAAGACTACCCGCAAGAGCTGCAGCCAACGCCA
CAGCAAAAATTAAAAATGACTCAGGAAGCGAATATTTGACTTTATTTAGTTTTGGCTTTT
GCATCGTGATTCCTTAACAAATTTCATAATTTCATCAAATTCATGCGCAGCAAGTTTAAG
CTGGTGTTCCTTTTTAATCTTTTTGCACTGTGCTTTCCAATCACGTACGCGTTTACGATA
ATGTCTTCCTTGATACCAGTATCCTACCCAATTTACAGGTACTAATAAAAATGGAACTAT
CATTGGAAGAATTAATGTTGACGCAAATATTATATTAGATTCAATATCAGTCATAACATC
TATAACTATTCCAACCCCCAGTAGAATTACAAATGATACAGCTATTACAGGACCTATTAA
TACATCGGTAGAAATTATCTGGCGCTTTAATTCATACTTTAAGGGTTTACTTGGAAGATG
CATTGATGGCTTTGACATATTCTCTACATTCCTTAACAAATTTTTCTAATAATAAATCGC
TTTCAAAATTGGGATTTTCTACGAATTTATCAAAAAGATCATCAACAATAGTTAAGATAT
TCTTTTTACTAAGAATACGTTTATTTTCATGCTTCGTTTCAGAATCAACTATAAGAGTAA
AGAAATATTTCTTGCCCTGAAATTTTACAGTAGTATCAATATAGAATAAATTTGACTTTT
GTAAATTACGTTTAAACCATGCGTCACTTAAACTATAAACGCCGAGATAATCATAATCGT
CGTTTAAATAACAGACCGTCCATTCAGGAGAAACAAAATCAGTAAATTCAGCATCAAAAT
CGCATGTTAATAAATGAATTGATTCAATACTGTTAATAAGTATTCCAGGACGTATTAAAG
ACTTTTTACCTCTAGAAAATCTTCCAGAAAGACTTTCATCAGTTTCATATGAAGAACCCC
AATAATAATTACGTCCTTCTGCCATACGTTTAAGAGCATTCAATAATTGGTCTGGAACAT
CAACTTGTCTTTGGAACTCTTCAAACATTGAATTGAAATCACTTTGCATTTTCATTCCTA
TTTACTCCAAGTAATAGGGGCCGAAGCCCCTTATCATTATTTCAGAGAATTAATATATTC
CTGAACATCGGCAGAGGTAGTTTCAACCCCAGAAATATTGCCATTAAAGGTTTCAACTCG
AGCAAGAGTATCTTCAATATCAACCTTAGTCAGCGCTGCAATTTCAACTACATCATCAGC
AGTACTAATTCCAAGGGCATTTGCTGCACGAGTTTCACGGATATATTCCAATTTAACCGC
AAGTTCTTGACGAGCATCATCTAACTCAACTACTTTCTTAGCGATTTCAATTCGCATTTC
AGCATAACCATCAGCTTTAGTAGTCAACTGCTCAGCTGTTCGACGATATAGTAAACCAAG
TTTAGCATGCATTGTTACATCTTGACCTTCGGAAAGAAGCTTGCGAATTTCACGCTCTTT
TGATTCGGCTTGTCGATTCTTTTCAATAACAAGTTCACGAATACGTTTTTCTTCATTAAT
AGATTTAACAGAAGCAGTTTTTAGGTCTTTAATTTTATCAAGTAATTTTGCTGCTGCAGC
AGTATACTGTTCTTCAACAGATAGATTTTTAGCCATAGCAGAACCAAGTTTAGTGCGAAT
AAACTCAACAATTTTCTTCAGTGTGTTCATAGTATTTCCTTTAGTTAATTAAGTGTTTTA
TAATCCGTGGGAGTATTATACTCTACTCCCAAGAGTTTGTAAACTTATTCTTCGTCCAAT
TCATCAATAAATTTATCGATACTACTATTAATAGACTCATCGATGATTAACAAAGCATCA
TCATCCATAATATTAATATTTTCTAAAGCATCAATATAAGCTTGATTGACTATAGACCAT
GCCCTTTGAAAATATGGGGCTTCATCATCAGGACTGAACCAACGTACACCTTCAAAGATA
CGTTTGGCGTAATCTAACACCCATTGTACAGGCATGTTTTGAGAACGTTCGTTAAACTCT
TTAAAATCCTTAGATTCAAAAAGCTCTTCAGGATAATTATTTCTATTACAAAAAGCTTTG
CTAAAGTTGCGTTTCATAATGTTCTCCTCAAAGTTATAGGCTCATAATATCTCAATCATG
AGCCTGTGTAAACTTATTTCATATTATTGAAATATTCTTCTGCGATTTCGTCGTTATCAT
GGTAAACTTTAGAAGACAGTTTAACATAACCTTCTGCCGTGAACATATTAATCACAACCT
TTACAGTATACCACTGACCGTCTTCATTACCCATTACTGCATAGGTTTCAAACATCGGAT
GATCAGGACCGATAACTTTAATATCATTCACTGTACGACCGAAATCTTCTGAAACACATT
TCATAAAAAAGTTGAACAGTTCGCCGTAATTATCCATTTCGTTCTCCAAGTTGTTTTCTG
TATCAGTAGTTGATATTGATATAGTACCACGGAAGAACAGGGATGTAAACAGTTTTGTGA
AAAAATTTTTAAAAAGTTTTGGGGAATTCTAGGGCAGGGAGAGGAAATTAAAAGGTAGGA
TAATATATTATAAAGGGTAGAAACTAAATGATGTCTAGAGAGGCCTGGAAAGGCCTAGAT
ACCAAAAAGCCCCAACCTTTCGGCTAGGGCTAAGAATGTTATTTGATTTGTTTAGCAGAC
CAAATGCGGTCTTTAATAATTTTTTGGATGTCTTCAACATACTCAAGAGAGTGAACATGC
GGGTTATCTTTGAAACTATAAGCACGAGCTAGCTTTTGGCCTTCAGTTTTGATTACTAAA
AGCTCTTTAAGGATAGCTTCATATTTGGAGATAATTCCTTTAACAATATTTTTCTCTTGT
GCGACTTTAGGATCTGCTTTAGGAGCAGGTTTACCTGAAGCCTTTGCAAAAGCAGCTCCA
GTAGCAACTAAACTTTTCCAAGCCATACTAACTGCATTCCCTGTAAATCCTTCGGCTTTC
ATATCACGAGCAAATTGGAATCGTGATTCATCGGAAGCATCTTTATAGGAATATTTACCC
GCGGCAATGGCAGCTTTTGCAACAGCTTGAATTTCGGTGCTAGATGCTTCATTTAACACC
GCTTCATTTAAAAATTGAGCATATGATTTCATCTTATTTCCTGTTTTAATTCGTGGTTTT
AATATACTTATTTATACTAAAAAGCCCCAACCTTTCGGTCGGGGCTAAGCCTTGCGGCAA
CCTTGTCGGGGTTCCACCTGCTAAGGCAAGTGTTTGTACGAAACGCCGGGATTCGAACCC
GGTTATTAAGCAGTTGACGCTACTCAATATTTTTAAAAGGCCATATCTCGACCATATCCG
AACGTTCCGTCAAAAACGCTACTCGGCTTACGGCAAAGATATTTCCTCGAATCGATAATT
CTGTGCGCCGTTTCTGCTGTGATGTAAGGGGATATTAACGAATCATAAAGATTTATTAAG
ACCAGTCCTTACTCAGGGAACATCAGTCCGACGACTTGCCGGTAGCGACCCGGCTTCTTG
TCTGGTCGAGGCAGTAGGGATCGAACCTACGACCCAGGACTTAGAAGGTCCTTGCTCTTC
CTTCTGAGCTATGCCTCGGATTTGGCGGACGTGATAGGATTCGAACCTATAACCAATCGC
TTAACAGGCGATCGCTCTGCCATTGAGCTACACATCCAAATTGGTGGGGAGTGATGGAGT
CGAACCACCCGAGTCGCAATGACAATGGATTTACAGTCCACACCGCTACCTCTACGGGAT
AACTCCCCTAATTAATTTGGTGGCCCTGGGTGGAATTGAACCACCATCTGGCGATTATGA
GTCGCTTGCTTGAACCTTCCAGCTACAGGGCCTTGGTGCTGATTGACGGAATCGAACCGC
CGACCTTCTCATTACAAGTGAGTTGCTCTACCTACTGAGCTAAATCAGCAAAACTGGCGG
AGGCGATAGGATTTGAACCTATGAGTCGCCGGAGCGACTGCCGGTTTTCAAGACCGGTGC
ATTCAACCACTCTGCCACGCCTCCAGTCTCCATACAAGGATTTGAACCTTGGACCTCCTG
ATCCCAAATCAGGCGCTCTACCAAACTGAGCTACACGGAGTAAATTAAATTGGAGCGGAT
AATGAGAATCGAACTCACATCATCAGATTGGAAGTCTGAGGTAATGCCATTATACGATAT
CCGCAAATTTGGTGCGAGAAGTGGGACTCGAACCCACAAGGAAATCATTCCGCAGCATTT
TAAGTGCTGTGCCTTTACCAATTTGACCATTCTCGCGCTGGGAATAAAGGACTCGAACCT
TTGCATCCTGGAATCAAAATCCAGTGCCTTACCAACTTGGCTAATTCCCAATTATTAACA
AAGGCTCTCAAACAAGAACCCTTGATGATAGAGGGTATTAATCAGTTCGGTATGAGTTAA
TAATAACAAATATTTCTTAAAGCAAATCAATCATTTTAACAGTTGGCAAAACGATTTCTT
CTTCAATATAAGCGAATTCAACACGTTCAACTACACGCAAGAAATAGTAATCATAGTATT
CAGAAGCATAACCAAGAATATTAATGAGACCTTCTGATACCAAATATCCATTCTCATGGA
ACAAGTCGAATACGCGGGCTTCGTCTTCTTCAGATTTATCACCAAGGTCAACTTCAATAT
CAAAGACCTTTTTAATGAATTCCGGGGTGAGCTGATCAGAGAACAGTCCTTCAACCAAGT
ATTCATAGATAATAGATGAGCAATCGCTGAATAGTGTATTACCACAATAATGTTCAGCTT
TGGTACTAGAATGACGACTCTTAAACTTCTTTAAGAGATATTTAAACTGTTGGAGCTCAT
GCTCTTCTACGCCAAACAGTGTTTTGGTTTTATAGTTATCACCATCATTTTCCCAGGTAG
TGACATCAATTACGTAACCCTTAGGAATTGTGCTACCTAAACGAATATACATTTGCTCAC
CATATTAATGTAAGTATATTTAATACATTCAAGACCCAAAGGATTCTTGAAAATATCATA
TTCAAGAGGACCTTTTTCCGTTTCAATAAAGAAATCAAAGTTAACTGTGTTAAATTTACG
GTCTTCCTTCACTAATTTAACTTGAGAAGATGAACGGTCAATGTAAACCTTTTCAACTTC
AAAACATGTTAAAATGCCATAATCATCGATCAAGGCTTTAGCTGCTTCTTGATCATATTT
ATATCCATTTTCAATGGACGATACTTTCGCATAAAGAATCATTATCAGCCTTCATCAACA
ATAGTATGAGTATGGGCATTTACAATTTGCCACCAATCAAAGCGATTGAAACCATACATC
GGTTTGTTTTCATTTTCTTTGATAATATCACGAAGTTCATCTTCAGTAGCGGCTTTCGCG
ATTAAATCAGCATATCCACCACGAGGATAATAATTATCACCTGCAAAAAGTAAAAAGTTT
GGTTTAATGTTATTGAACTCTTCAAGAGTAATAACTGTTCCTGATGGTAGTTGAACAATC
TCTACATATTTATATCCTAATTTTTGAGCAGCATCTACTGCTTCAAGCAAAGTATCATGA
GCCCATCCAATTTGACAATTTGGCTTTTTAGATGGATTAGCCTGATAATCCGCATACACT
TTATACATTCTATTCACCTTTAGAAATTTTATCCATAACGATAGCAATTAAACCAATTAA
AAATGCTACTACAAGTGAAAACACATTTTCTGCTGTAGTCAATAATCCGCATATAAATCC
AACAAACATTGAAAAACTAAAAGCGGAAGCAGAAATTGCAATAGCAACATTTCGAATTAA
TTCACAACGTTTCATTTTATTCTCCTCAGTAGTTGATAGGGTAATAGTATCACTACCCCA
TCTAAAAGTAAACGGTTTTATAAACTATTTTATTCCGCGTGATGCAATTGTGTTCTGCCG
TTTAACACCAGTTTGTTTAAAGTCATGATTGTCGATTTGATCGGTACGAGCAACTCCGAA
TGATGATTTGATTTTAGCTTTAGATCCTTTAGTTATAGAAATAAACATCTCGGAAGCCCC
TTTCTTAAAGTCCGAGGCGATTACTTCAACGCCGTTCTTAGCAAGTGACTTCAGAATTGC
CTCTGCATACTCATCGTCAAGACTCGTCTGAGTATTAATAACGAATGTCTTTGGCGCAGC
CGCTTCATTGATAAATTCTTTAAATGTTTTCATTTTATTTTCCTAATTAATTTTGATAGG
ATAATAGTATCACTACTAAAACCCCATGTAAACAACTTTGTGAAATTATTTTAAATCTTC
TAATCGTTTCTTCATCTTAGAACCGTTCTTAGCGATTTCGCCTGCATCAGAGCATAATGC
TAAAAGCGCTTTAACTTTAGTTTGGTCTCCTTTAAAAGATGAAGGATCGACTGCAGCAGC
AAGTTCTTTAATACGGGAGAAAAGACGCTCAGCTTCAGCTAAAGCCATTCCAAGCTTTAC
TTCCATACCATGAGAAGATTCAGTAATAGTAGTTTTTGTAGCAAACTCTTTGAAAGTTTT
CATTTTATTTTCCTAATTAATTTTGATGAGGTAATAGTATCACTACCTCATCAGTATGTA
AACAACTTTATGAAATTATTTTAAATCATCTGCCCAATCGAGTTTAAGAGGCTCTTTGTA
TTCACGGTCAAGTACAACCGGAATTTGTACATCACCGCTAAATGATAAGGGCCCAACATT
ATAAGACAATGTTATATGCGGTGTGTAATCATCAAAATCATGTGTAGCACCTAGTGCCCG
CGCATACATGTGTCGACAGCGCAGATATTCAGAATCTAACACAAGTACAAGAGTCGATCC
ATCTTGTGTTTTCCATACTTCTAAATGTCCAGAAGAAGCTACTTCAAAACTTCCACTCGA
TGGAACATATGGAACATTTACTCTCGAATAACATATAGTCGAATGAATTTTTTCTCTAGG
AACTGGATTAGGAACACGTAAAGAGCGCTGGAGTTCTTCCAGCGCGTCAAGTGTTAATTC
TGAAAACTTAGCTGCTACATAAAGACCCGTTGAAAAGTCTTTAAATTCCATCATTCTTCA
TCTTTTGCTTCATCTGCAGATTCAGCAGTAAGATTCTTAACAGCTTCAACGATTTCTTCA
ACTTTAATGGTATCGCCAGTGATACCTACTGCACGAGCAATTTCAGCCAAAGTTCCTTGC
AGAAATTTGGATTCTTCCATCAGACGAGCCGCTTGGTCCTGCGTATCAAGAATGCGAGAT
TTCAGAGTTACGATTTCAGCAGACAGTTTTTGTTCAATAGTTTGTTCAGACATTATAGTA
CCTTTAGTGTATTTTTAATTTTAGAAAAAAGTTCTTCAAGAGAACCATCGTTTGTAATTA
CTAAATCGCCACCACGAATTGGCAATCCAGCTTCTGTAATGTGTGTATCATTGGATTTTT
GACCAGGACGAACTACATGAATTACTGTAGCACCCATCGCCCTAGCCGCATCCATTTCAT
GATCTTGACGAGTATCAGGAACGATATAATAATCATAACCCGAGTTAAATTTATCAAGAT
AATCTAAAGCAAATAATTTTACCCAGTACATGCGGTCGAAGTTATTAACAATCAAATCCG
TACCTAGGGCTTGCATCAGACGACGGACTGACCATTGATCTTCAATATTATTTATAACGT
CAGTAATTTTGTTAAATGCTACGAAATTAACTGATTCTTTTCCTTCGTCATCAAAAACAA
ACACACCTTTAATTGGGCTTTTACCATTAAGATAGCAAAATGCTTGTTCCATAATCGCGA
TTACTTCTGATTTAGTCAGATTTAAATTAGTCTCACGATCATAGTCAATTCCTTCAAACT
CTTTACGAGTTAAGCAAGGATAGTCAGTGTTTGCTGCAAATACTCCCCATGCATAAGCCA
ATGCATCCTTAATAGGACCAGCAAGTTGGTATTTAACTGCAGAATAATTGCTCATGATAA
AATCAGCAGTAGTATCTTTTCCACTACGCTTTACACCGCTTAAAAAGATTAGTTTCATGT
GTTTCTCCTCAAATTTAATTAAGATTATAACACACAAAACTGAAGCATTAAACTTCTGCT
ATAATTTTTCCATCTTTTTCTACTTGAAAATAGGTGTAAGGAATCGTTGCAGTACATACT
AAAGCCGGGTCTGAATCTTCCGTATAGCTAAATTCTACTTCAGACAGGTCAGAAACCCAA
GGCTTATAAAAATTTATTGACATCACGATTTCAGTTTTGCTATTATCTAAGATGTAAAGC
GTAATGTACTCAGGACCTGTTTTTTGGGCAGTATTTTCACCTGTAAGATAGTTGCTAGTT
CCTAGCATCCATTCATACATTCCTATCCACGACTTAAGTTCTTCATCAACTATAAATCTC
ACAATGAGTGGGTCATACTCGAATGTAACACCTGGACGTTGTGCTCGGCCCAGTCCAAAC
GGCCCAGTCACGGTATCAGTAACAGGTATTCTAATTCCTGGAATAGGAACTGACTGAGCA
TTTAAAGTAAAAGCAGATGTAGTATTACTATGCGGTATTGATACTACAAAGTTAGTTGTA
TTTGCTTGGTTAAAAATTTGTTGCAGAGCTTGCGACATATATTCCTCATAATGCTTTATA
ACTGTTGGTGGTATAATGGGTCTAAGTCCCTTCCATTCAATTCCATTTAGAACAAACAAC
AGAAAAGAATGGAAGATAATAGAATTAGATATTTGACCAGACTTTGTTTGCAGAGAAACG
TTTTCCTTTTGAAACGAACTGCTGAAGTGGCATCAACACAACGTTCGCCCAGTCTTTCGG
GGCGATTTCAACAAGGCTACCCATAATATTACCAGGTATATACGCCTTAATCATTTGGTC
TGCACCCCTAAATCCTTTCACTTGACTCCAATCAATTTTTAATTTCGTTTTATTAGTAAT
AGTAGGTGTATTTGCATATTGCTTTAAAAGCTCTTCTAGAAATTGCTGACGAGCTTTAGG
TGGAATATAGTGCAAGTTTAATCCGTACATTAAATTATGCTTACCTAAACCAAGATAAAT
TATCAAAGGAAATTTATCCCAGTAAGGAAGAGTTTCCTTGTGTTTAGCATCGTAAGCAAA
AGCATATATTCGTCCCGGCTGTGGGCGAACAACTTTATGTCCTTTTACTTGCTTAATAGT
TTCAGCAAACCACTTTCTGGTTTTATTATTAATTGCTGCACCTTCATTACGAATTTTATC
GCGTAATGTTTGTCTGAATGAATTTATCATAAGCAGTTGTCTTTCTTGCTTATTGAGTTT
ATTCATTGGTTTTGATTCAAGCTTTTGAATCTTTTCAGCCGTTTTAATTCCTGAAGCATA
TTTTGACATTGCTGAAGTAAACGTAGAGTATTTGATTCCTCTTTCTTCAGCAAATTGCTT
TCCTGTCATTCCTTTTGCTTTGGCCTTTTTATATTCAAGACCTATCTGAATCCATTTCTT
TTCGTTTAATGATTGCTTAACCTTTGGAACTTGGGGAGTGCTTTCATTAATTATTTGAAA
AATAGCCATTATGCCCCCTTAAAGCCAAGAGCTCGTAATCCATCTTCTGTTAGAATTCTA
AATTTTATTCCACGCTTTTCAGCTAAAGATTGTGCTGCTTTCCATTTGTCAGTATTAACA
GAATATGTATAAATTTCATTCATAAATCTTTTCTTCGCTGCGGTTGTTAGATGTGCTGGT
TTAACTGGTGGTTGTGTTTCTTTTTTAGGTTTTATTTCAATAAAAAATTCTTGTCCAGAA
GAATCTTTCATCCAAATATCCATGAAGTATCTACGTTTTTTCCCTTCTGCATTACAAAAA
TAAGGAATTACTGCTGTTTCACTACCCCATGCAATAATTTCTGGATTTTTATCTAACCAT
TCAAAAAAGAATTTTTCCCAATTTGATCTATACGTAATTTTTTTAGGGTCACCTCTATAT
TTTGATATATTTTTAGGAACCCATTTTCCAGAATATGCCATTGGATTCTCCTTATAAATA
GATAATATATTTATAAACAGGAGGGCCCATGCTCTTTACATTTTTTGATCCGATTGAATA
TGCGGCCAAAACGGTGAATAAAAACGCGCCGACTATTCCTATGACAGATATTTTTAGAAA
CTATAAAGACTATTTTAAACGCGCTCTTGCGGGATACCGCTTACGTACTTATTATATCAA
AGGTTCACCACGCCCGGAAGAATTAGCAAATACTATATATGGAAATCCACAGTTGTATTG
GGTTTTATTGATGTGTAATGATAATTATGATCCGTATTATGGATGGATTACTTCGCAAGA
AGCTGCTTATCAAGCATCTATACAAAAATATAAAAATGTAGGTGGAGACCAAATAGTATA
TCACGTGAATGAGAATGGTGAAAAATTTTATAATTTAGTATCATACGATGATAATCCATA
TGTTTGGTATGACAAAGGCGATAAAGCTAGAAAATATCCTCAATATGAAGGAGCACTTGC
TGCTGTTAATACGTATGAAGATGCTGTTCTTGAAAATGAAAAACTTCGTCAAATAAAAAT
AATAGCAAAATCGGACATCAATTCATTTATGAACGACCTTATACGTATAATGGAGAAATC
TTATGGAAATGATAAGTAATAACCTTAATTGGTTTGTCGGTGTTGTTGAAGATAGAATGG
ACCCATTAAAATTAGGTCGTGTTCGTGTTCGTGTGGTTGGTCTGCATCCACCACAAAGAG
CACAAGGTGATGTAATGGGTATTCCAACTGAAAAATTACCATGGATGTCAGTTATTCAAC
CTATAACTTCTGCAGCTATGTCTGGAATTGGAGGTTCTGTTACTGGACCGGTAGAAGGAA
CTAGAGTTTATGGTCATTTTTTAGACAAATGGAAAACTAACGGAATTGTCCTTGGCACGT
ATGGTGGAATAGTTCGCGAAAAACCGAATAGACTTGAAGGATTTTCTGACCCAACTGGGC
AGTATCCTAGACGTTTAGGAAATGATACTAACGTATTAAACCAAGGCGGAGAAGTAGGAT
ATGATTCCTCTTCTAATATTATCCAAGATAGCAATCTAGACACCGCAATAAATCCTGATG
ATAGACCACTATCAGAGATTCCAACCGATGATAATCCAAATATGTCAATGGCTGAAATGC
TTCGCCGTGATGAAGGATTAAGACTAAAAGTTTATTGGGATACCGAAGGATATCCGACAA
TTGGTATTGGTCATCTTATCATGAAGCAGCCAGTTCGTGATATGGCTCAAATTAATAAAG
TTTTATCGAAACAAGTTGGTCGTGAAATTACTGGAAACCCCGGTTCTATTACAATGGAAG
AAGCGGTAACTTTATTTGAACGTGATTTAGCTGATATGCAACGTGACATTAAATCACATT
CTAAAGTTGGCCCAGTTTGGAAAGCAGTTAATCGTTCTCGTCAAATGGCGTTAGAAAATA
TGGCATTTCAGATGGGTGTTGGTGGTGTAGCTAAATTTAACACAATGTTAACTGCTATGT
TAGCAGGAGATTGGGAAAAAGCATATAAAGCCGGTCGTGATTCATTGTGGTATCAACAAA
CAAAAGGCCGTGCATCCCGTGTTACCATGATTATTCTTACGGGAAATTTGGAATCATACG
GTGTTGAAGTGAAAACCCCAGCTAGGTCTCTATCAGCAATGGCTGCTACTGTAGCTAAAT
CTTCTGACCCTGCTGACCCTCCTATTCCAAATGACTCGAGAATTTTATTCAAAGAACCAG
TTTCTTCATATAAAGGCGAATATCCTTATGTGCATACAATGGAAACTGAAAGTGGACATA
TTCAGGAATTTGATGATACCCCTGGGCAAGAACGATACAGATTAGTTCATCCAACTGGAA
CTTATGAAGAAGTATCACCGTCAGGAAGAAGAACAAGAAAAACTGTTGATAATTTGTATG
ATATAACCAACGCTGATGGTAATTTTTTGGTAGCCGGTGATAAAAAGACTAACGTCGGTG
GATCAGAAATTTATTACAACATGGATAATCGTCTTCACCAAATAGATGGAAGCAATACAA
TATTTGTTCGAGGCGATGAAACTAAGACGGTTGAAGGTAATGGAACTATCCTAGTTAAAG
GTAATGTTACTATTGTAGTTGAAGGTAATGCTGACATTACAGTTAAAGGAGATGCTACCA
CTTTAGTTGAAGGTAATCAAACTAATACAGTAAATGGAAATCTTTCTTGGAAAGTTGCTG
GAACAGTTGATTGGGACGTTGGTGGTGATTGGACAGAAAAAATGGCATCTATGAGTTCTA
TTTCATCTGGTCAATACACAATTGATGGATCGAGGATTGACATTGGCTAATATACTTCCA
ATGAGCGCTGATTTAGGGGAATCCATGGAAGGTTCTTCTATCGACGTCACCTTTACCGCT
CAATTAGAAACAGGTGAAACGTTAGTATCTATAAATATAACTAGTTACGAAGAAACTCCT
GGGGTTTTAGTAGAAGAAAATCGCTTATATGGAACATATGAATCTGTATTTGGTTTCGGA
AATGACGCGTTGAAATATCGTTTAGATGATGAATTTAAAACTGCTGCTTCATGGGAAGAC
CTTCCAACTGATTCTGACACTCAGTTGTATTTGTGGAAAGCTCCTCAGAATCTCCAGAAG
ACATTCACTTACGAAGTAACATTAATATATGACTACCAAGAACAAAGTGAATCTGGGGGT
TCTGGCAGTAATTCTAGGTCATCTTCTGATACTACTGAACCAACGAATCCTCCAGCTCCA
GTAAGAAAAACTTTAGTTAAAAATTATACTAAAACTATAGTTGGAAATTGGAGTCGTTGG
GCTAATAAATTGAGAAGCTATGTGTATGAGAGGTCATAGATGTCAGGATTAAGTTATGAT
AAGTGTGTTACTGCTGGCCATGAAGCGTGGCCTCCAACAGTTGTGAATGCTACACAAAGT
AAAGTATTCACTGGAGGAATTGCTGTTCTCGTAGCAGGTGATCCAATTACAGAACATACA
GAAATTAAAAAGCCATATGAAACACATGGCGGAGTGACACAACCTAGAACTTCTAAGGTA
TATGTCACGGGAAAGAAAGCTGTTCAAATGGCTGATCCAATATCATGCGGTGATACTGTG
GCTCAGGCATCATCTAAAGTATTCATTAAATAGGATTTAAAATGGCAAATACCCCTGTAA
ATTATCAATTAACAAGAACAGCAAATGCTATTCCCGAGATATTCGTCGGGGGTACATTTG
CTGAAATAAAACAAAACCTCATTGAATGGCTTAATGGCCAAAATGAATTTTTGGATTATG
ATTTTGAAGGCTCAAGATTAAACGTTCTGTGTGACCTTTTGGCTTATAATACATTGTACA
TTCAGCAGTTTGGTAATGCTGCTGTGTATGAAAGCTTTATGCGTACTGCTAACTTACGAA
GTTCAGTTGTTCAAGCTGCACAAGACAACGGATATTTACCTACTTCAAAATCCGCTGCGC
AGACCGAAATTATGTTAACATGCACCGACGCATTGAATAGGAATTACATTACTATTCCTC
GCGGAACTCGCTTTTTAGCATATGCAAAAGATACTTCTGTTAATCCATATAACTTCGTTT
CTACCGAAGACGTTATTGCTATTCGTGATAAAAATAACCAATATTTTCCACGTTTAAAAC
TTGCTCAAGGACGTATAGTAAGAACTGAAATCATTTATGATAAATTAACACCTATTATCA
TTTATGATAAAAATATTGATAGAAACCAAGTTAAATTATATGTTGATGGAGCTGAATGGA
TTAACTGGACGAGAAAATCGATGGTTCATGCTGGTTCTACATCAACAATTTACTACATGC
GCGAAACGATTGATGGAAATACTGAATTTTATTTTGGTGAAGGTGAGATTTCCGTTAATG
CGGCAGAAGGAGCTCTAACTGCTAACTATATTGGAGGTCTTAAGCCTACTCAGAACTCAA
CTATCGTTATTGAATATATCAGTACTAATGGCGCAGATGCGAACGGAGCAGTCGGATTTT
CATACGCAGATACATTAACAAATATAACTGTCATCAATATTAATGAAAATCCAAACGATG
ACCCAGATTTTGTTGGGGCAGATGGCGGCGGCGATCCAGAAGATATTGAACGTATTCGCG
AATTGGGTACTATTAAACGCGAAACCCAGCAACGCTGCGTAACTGCGACTGACTATGATA
CATTCGTTTCAGAGAGATTTGGTTCTATTATTCAAGCCGTTCAGACTTTCACTGATTCTA
CTAAACCTGGGTATGCATTTATTGCTGCTAAACCTAAATCAGGATTGTATTTAACTACTG
TGCAGCGTGAAGATATTAAAAATTATCTCAAAGACTATAATTTAGCTCCTATTACGCCAT
CAATTATTTCTCCTAATTATCTTTTTATTAAGACTAATTTAAAAGTCACATATGCTTTAA
ATAAGCTGCAAGAATCTGAACAGTGGCTTGAAGGTCAAATAATTGATAAAATTGACCGCT
ATTATACTGAAGATGTAGAAATTTTTAACTCGTCTTTTGCCAAGTCTAAGATGTTAACTT
ATGTAGATGATGCAGATCATTCTATCATTGGTTCATCAGCGACTATTCAAATGGTTCGTG
AAGTACAAAATTTCTATAAAACTCCTGAAGCGGGTATTAAATACAATAATCAAATAAAAG
ACCGTTCTATGGAATCTAATACGTTTTCATTTAATTCTGGACGAAAAGTTGTAAATCCTG
ATACTGGTTTAGAAGAAGATGTATTATATGACGTTCGCATAGTATCAACAGACCGAGATT
CTAAAGGAATTGGTAAAGTTATTATTGGTCCATTTGCTTCTGGCGATGTTACAGAAAATG
AAAACATTCAGCCATATACAGGCAATGATTTTAATAAATTAGCAAATTCTGATGGACGCG
ACAAATACTATGTTATCGGTGAAATAAATTATCCAGCTGATGTGATTTATTGGAATATCG
CTAAAATTAATTTAACATCTGAAAAATTTGAAGTTCAGACTATTGAATTATATTCTGACC
CAACCGATGATGTTATCTTTACTCGCGATGGTTCACTGATTGTATTTGAAAATGACTTAC
GTCCACAATACTTAACTATCGATTTGGAGCCTATATCACAATGACAGTAAAAGCACCTTC
AGTCACTAGTCTCAGAATTTCCAAGTTATCCGCAAATCAGGTGCAAGTACGCTGGGATGA
CGTTGGTGCTAATTTCTACTATTTTGTAGAAATCGCTGAGACAAAAACAGACTCGGGGGA
AAAACTCCCGAGTAATCAATATCGTTGGATTAATTTAGGATATACAGCAAATAATAGTTT
CTTTTTTGATGATGCTGATCCATTGACATCGTACATTATTAGAGTAGCTACAGCTGCGCA
GGATTTTGAGCAGTCTGATTGGATTTATACCGAAGAGTTTGAAACTTTTGCTACGAATGC
TTACACATTTCAAAACATGATTGAAATGCAATTAGCTAATAAATTCATTCAGGAAAAGTT
CACTCTTAATAATTCTGATTATGTTAATTTTAATAATGACACCATAATGGCTGCATTGAT
GAATGAATCGTTTCAATTCAGCCCATCATATGTTGATGTTTCATCAATCAGTAATTTTAT
TATTGGTGAAAATGAGTATCATGAAATACAAGGTTCCATTCAGCAAGTATGTAAGGATAT
TAACCGAGTTTATTTGATGGAATCAGAAGGGATTCTATATCTTTTTGAGCGCTATCAACC
CGTAGTTAAAGTATCTAATGATAAAGGACAAACCTGGAAAGCTGTAAAGCTCTTCAATGA
CCGTGTAGGATATCCTTTATCTAAGACAGTATATTACCAATCTGCGAACACTACATACGT
TCTAGGATACGACAAGATTTTCTACGGCCGTAAATCTACTGATGTTAGATGGTCAGCTGA
TGATGTCAGATTTAGTTCACAGGATATAACATTTGCTAAACTTGGTGACCAATTGCATCT
AGGATTTGATGTAGAAATTTTTGCCACTTACGCGACTTTACCTGCGAATGTATATCGTAT
AGCTGAGGCTATTACTTGCACGGATGATTATATCTATGTTGTTGCCAGAGATAAAGTTAG
ATACATAAAAACGAGTAATGCACCCATAGATTCTGATCCATTATCCCCAACATATTCGGA
AAGACTTTTTGAACCCGATACAATGACTATAACCGGAAATCCTAAAGCAGTATGCTATAA
AATGGATTCTATTGGTGATAAAGTTTTTGCTCTTATTATTGGTGAAGTTGAAACATTAAA
TGCCAATCCGAGAACTTCAAAAATAATTGATTCTACTGATAAAGGAATATATGTTTTAAA
TCATGACACGAAAACATGGAAAAGAGTTTTTGGTAATACCGAAGAAGAAAGAAGACGTAT
TCAACCTGGATATGCGAATATGTCAACTGACGGTAAATTAGTTTCTCTATCTTCGAGTAA
TTTTAAATTTTTAAGTGATAACATTGTTAATGACCCTGAAACTGTGGTGAAATATCAGTT
AATCGGTGCTGTTAAATATGAATTTCCTCGTGAATGGTTAGCTGATAAGCATTATCATAT
GATGGCATTTATAGCGGATGAAAAATCTGATTGGGAAACTTTTACGCCTCAGCCGATGAA
ATACTATGCAGAACCATTCTTTAACTGGTCTAAAAAATCTAACACGCGCTGTTGGATAAA
CAACTCTGATAGAGCTGTAGTAGTTTATGCTGATTTAAAATACACCAAAGTTATAGAAAA
TATTCCGGAAACATCACCAGATAGATTAGTTCATGAATACTGGGATGATGGTGATTGCAC
TATAGTAATGCCAAATGTTAAATTCACTGGATTTAAAAAATACGCATCAGGAATGCTTTT
CTACAAAGCCTCCGGTGAAATAATTTCTTACTATGATTTTAATTATCGTGTGAGAGATAC
AGTCGAAATTATTTGGAAACCTACTGAAGTATTTTTAAAAGCATTTTTACAAAACCAAGA
GCATGAAACTCCTTGGTCGCCTGAAGAAGAGCGTGGATTAGCTGACCCCGATTTAACACC
ATTAATTGGCACAATGATGCCTGATTCTTATTTGTTACAGGATTCGAATTTTGAGGCATT
TTGCGAAGCATATATTCAATACCTTTCTGATGGATATGGAACTCAGTATAACAATTTACG
AAATTTAATTCGTAACAAATATCCACGAGAAGAGCATGCATGGGAATATTTGTGGTCAGA
GATATATAAAAGAAACATTTATTTAAATGCTGACAAACGTGATGCTGTTGCAAGATTTTT
CGAATCACGTAGCTATGATTTTTATTCTACTAAAGGAATTGAAGCATCATACAAATTTCT
TTTTAAAGTTCTTTATAATGAAGAAGTTGAAATTGAAATTGAATCTGGGGCTGGTACTGA
ATATGATATAATCGTTCAATCTGATTCTTTGACTGAAGACTTAGTAGGACAAACGATTTA
TACGGCAACAGGAAGATGTAATGTTACTTATATAGAAAGAAGCTATTCTAATGGTAAATT
GCAATGGACCGTAACTATTCATAATCTTTTAGGAAGATTAATTGCTGGTCAAGAAGTTAA
AGCAGAAAGACTCCCTAGTTTTGAAGGCGAAATTATTCGTGGGGTTAAAGGAAAGGATTT
GCTTCAAAATAATATAGACTATATTAATAGAAGTCGATCATATTATGTAATGAAAATTAA
ATCCAATTTACCTTCTTCTCGTTGGAAATCTGACGTTATTCGTTTTGTTCATCCAGTAGG
ATTTGGATTTATAGCAATTACCCTTTTAACAATGTTTATTAATGTTGGTTTAACTCTTAA
ACATACAGAGACTATAATTAATAAATACAAAAACTATAAATGGGATTCTGGATTGCCTAC
TGAATATGCTGATAGAGTAGCTAAATTGACTCCAACCGGTGAAATTGAACATGATCCAGT
AACAGGCGAAGCAATTTATGAACCTGGTCCAAAGGCTGGTGTAAAATATCCTCTTCCTGA
TGACTATAATGCTGAAAATAATAATTCAATATTTCAAGGTCAATTGCCGTCTGAACGACG
TAAATTAATGAGTCCTTTATTTGATGCATCTGGAACAACATTTGCGCAATTTAGAGATTT
AGTTAATAAACGTCTAAAAGATAATATAGGAAATCCAAGAGACCCTGAAAATCCAACACA
GGTTAAAATAGATGAATGATTCAAGTGTTATCTATCGTGCGATAGTTACTTCAAAATTTA
GAACAGAAAAAATGTTGAATTTTTATAATTCAATTGGAAGTGGTCCGGATAAAAACACTA
TCTTTATCACATTTGGAAGATCAGAACCATGGTCATCAAATGAAAATGAGGCGGGCTTTG
CCCCGCCTTATCCAACCGATTCTGTATTAGGTGTAACTGACATGTGGACTCATATGATGG
GAACAGTAAAAGTTCTTCCATCAATGCTTGATGCTGTTATTCCTCGCAGAGATTGGGGAG
ATACTAGATATCCGGATCCATACACATTTAGAATTAACGATATTGTAGTGTGTAACTCAG
CTCCTTACAATGCTACTGAATCAGGCGCAGGCTGGCTAGTATATCGTTGTTTAGATGTTC
CTGATACTGGAATGTGTTCAATAACATCTTTAACTGATAAGGATGAATGCCTCAAATTAG
GTGGAAAATGGACTCCTTCTGCTAGGTCAATGACTCCTCCTGAAGGTCGAGGAGATGCTG
AAGGAACAATTGAACCTGGGGACGGATATGTTTGGGAATATCTATTTGAGATTCCACCTG
ATGTATCTATAAATAGATGCACAAATGAATATATCGTGGTTCCTTGGCCTGAGGAATTAA
AAGAAGACCCGACTAGATGGGGATATGAAGATAATCTCACTTGGCAACAAGATGATTTTG
GATTAATTTACCGTGTTAAGGCAAATACTATCCGTTTTAAAGCATATTTAGATTCAGTTT
ATTTTCCTGAAGCTGCATTGCCGGGAAATAAAGGATTTAGACAAATATCGATAATCACGA
ATCCTCTTGAAGCTAAAGTTCATCCAAATGACCCAAACGTTAAAGCTGAAAAGGATTACT
ATGATCCGGAAGATTTAATGAGGCATTCGGGCGAAATGATTTATATGGAAAATAGGCCAC
CTATTATTATGGCAATGGATCAAACAGAAGAAATCAATATTCTGTTTACATTTTAAATTA
AGGGAGCCCATGGGCTCCCTTTTTCTTTATAAATACTATAAACTCATAAGGAAACCGCTA
TGTTCATTCAAGAACCAAAGAAATTGATTGATACCGGCGAAATTGGTAACGCTTCTACTG
GTGATATCTTATTCGACGGTGGTAATAAAATTAATAGTGATTTTAATGCAATTTATAACG
CGTTTGGCGATCAGCGTAAAATGGCAGTAGCAAATGGTACTGGCGCTGACGGCCAAATTA
TCCATGCTACTGGATATTATCAAAAGCATTCTATCACAGAGTACGCAACTCCGGTGAAAG
TTGGTACTAGGCATGATATTGATACCTCTACTGTTGGTGTTAAAGTTATCATTGAAAGAG
GAGAACTCGGTGACTGTGTTGAATTCATTAATTCTAACGGATCGATATCAGTTACTAATC
CTTTAACAATTCAAGCCATTGATTCAATTAAAGGTGTTTCGGGTAATTTGGTAGTAACTA
GCCCATATAGTAAAGTAACTTTGCGCTGTATTTCATCTGATAATTCTACGTCAGTTTGGA
ATTATTCTATTGAAAGTATGTTTGGCCAAAAGGAATCACCGGCCGAAGGTACATGGAATA
TTTCTACATCTGGATCAGTTGACATTCCATTATTTCATCGTACTGAATACAATATGGCTA
AATTGCTAGTTACTTGTCAATCGGTAGATGGAAGAAAAATTAAAACGGCAGAAATAAATA
TTCTTGTAGATACTGTTAATTCAACGGTCATTTCTTCTGAATATGCTGTCATGCGGGTTG
GGAATGAAACTGAAGAAGACGAAATCGCTAATATTGCATTTAGTATTAAAGAAAATTATG
TAACGGCGACTATAAGTTCTTCAACTGTCGGTATGAGAGCAGCAGTTAAAGTTATCGCTA
CGCAGAAAATCGGGGTGGCTCAATAATGAAACAAAATATTAATATCGGTAATGTTGTTGA
TGATGGTACCGGTGACTACCTGCGTAAAGGTGGTATAAAAATAAATGAAAACTTTGATGA
ACTTTATTATGAACTTGGTGATGGAGATGTTCCATATTCAGCCGGTGCCTGGAAAACTTA
TGATGCTTCATCTGGACAAACACTAACGGCAGAATGGGGAAAATCATACGCTATTAATAC
CTCTTCTGGAAGAGTAACTCTACAACTTCCTAAAGGAACTGTTAATGATTACAACAAGGT
AATTAGAGCTAGAGACGTATTCGCTACATGGAACATCAATCCAGTTACTTTAGTAGCTGC
TTCCGGAGATACAATTAAAGGGTCGTCATCGTCAGTTGAAATTAATATTCAATTTAGTGA
TTTAGAACTCGTTTATTGCGCTCCAGGACGCTGGGAGTATGTCAAAAATAAGCAAATTGA
TAAAATTATCAGTTCAGACATTAGTAATGTAGCTCGTAAAGAATTTTTAGTCGAAGTTCA
AGGGCAAACAGACTTTTTAGATGTTTTTAATGGAACTAGTTATAATGTCAATAACATCAG
AGTAAAACATCGTGGTAACGAATTGTATTACGGCGATGTATTTAGCGAAAACAGTGACTT
TGGCTCTCCTGGAGAAAATGAAGGCGAATTAGTTCCTCTTGATGGATTTAACATTCGATT
AAGACAGCCTTGTAATATTGGTGACACTGTTCAAATTGAAACATTTATGGATGGTGTATC
ACAATGGAGAAGTTCATATACAAGACGCCAAATAAAAGTACTGGATTCAAAACTAACATC
AAAGACTTCTTTAGAAGGAAGCATTTATGTTACTGATTTATCAGCGATGAAATCAATTCC
ATTTTCTGCATTTGGATTAATTCCTGGTGAACCCATTAATCCTAACTCTCTTGAGGTTCG
TTTTAATGGAATTTTACAGCAGCAGGCTGGAACAGCTGGATATCCTCTGTTTTTGTGTGA
AGGTGCTAATTCAGATACGCAAGAAGGATGTATATCTCTTGGTGGCGAATGGAAAGAATC
AAACACCGATTATTCTATAGAATATGAAGACGGAAAACCTATCAGTCTTTTATTTGATAG
AAAATTTGAATCCGGTGACATTATCGTTATAACATGGTTCAATAATGATTTAGGAACTCT
TTTAGAAAAAGATGACATCATTGAATTGACTGATGACCGTTATGTTAGTAAAGGATCATC
TACTGAAGTTACTGGTGATGTGGCCTTAACAGATTTTGATAAAATCGGTTGGCCAAATGT
TGAAAAAGTTGATTCTTATACTAGAACGTATAATTCAATATCATCTATTTTCGACAGCAT
TTATCCTGTTGGTTCAATTTATGAAAATGCTATAAACCCAAATAATCCGGTGACTTATAT
GGGATTTGGTTCATGGAAATTATTTGGTAAAGGACAAGTTTTAGTAGGATGGAATGATGA
TGTTACGGATCCAAACTTTGCTTTAAACAATAATGATTTAGATTCTAGCGGAAATCCTTC
ACATACTGCAGGTGGAACAGTTGGAACAACATCAGTAACGCTTGAAAACGCAAATCTTCC
TGCAACTAAAACCGACGAAAGAGTTTTAATTGAAGACGAAAATGGATCGGTTATTATTGG
AGGTTGCCAATATGACCCAGATGAAACTGGTCCTATATATACAAAATATCGTGAAGACTA
CGCAACAACAAACTCTTCACATACTCCTCCTGCTAATATTAGTAATATTCAACCGTCTAT
TACTGTATACCGTTGGATAAGGATTGCATAATGAGTTTACTTAATAATAAAGCGGGAGTT
ATTTCCCGCTTAGCAGATTTTCTTGGTTTTAGAACTAAGAAAAATGATATTTCGGTTATG
AATAATCAGCCAGTAGGTGCTGTAACAATTTCACAAATAGCAAAAGGTTTTTATGACCCA
AATGTAGAATCTGCTATCAATGATGTTAGAAATATGGCAGAGCAACAGGTTGGAGCTGTA
TTAGTTAATATAAGTGGCGTATCTCCTACTGGCGTGCAGCAAACAGACTATTGGTCATTT
GAAGGAACTGTCAATGATACTTCAGCCAAACCAGGTGACACAGTTATTGTTAACATGTTT
GGTATTCCAGTTAAAGCTACTAATGGAATGACTTCAATCGAATTTACCAGTGCCGTTAGA
ACAGCTCTACAGGAAATGGTTGCAAAATTTATTGCAATTGATTCATTTGAAGACCATCCT
ACCATAGGAAATAAAATACAAGTTAAGTATTTAGATAACCAAGAACATATCTTAGAACAA
TACTCTGATAAAGGAATTACTTTCAAACAAGAAATAATTTCTCCTTCTAAACCCGGATAC
GGAACTTGGCAATTATTAGGTGCGCAAACTGTTACGCTAGATAGTCACACACAACCTACG
GTATTTTATTATTTTGAGAGAATAGCATGAGTAACAATACATATCAGCACGTATCAAATG
AATCAAAATATGTTAAATTCGATCCAGTAGGATCGAATTTTCCTGACACTGTCACGACAG
TGCAGTCTGCATTATCAAAAATAAGTAATATCGGCGTAAATGGTATTCCTGATGCAACTA
TGGAAGTTAAAGGAATAGCAATGATTGCGTCAGAGCAAGAGGTTTTAGATGGAACTAATA
ATTCTAAAATCGTTACACCAGCTACATTAGCAACAAGATTATTGTATCCAAATGCTACTG
AAACTAAGTATGGTTTAACGCGCTATTCAACAAATGAAGAAACATTAGAAGGCTCAGATA
ATAATTCATCAATAACGCCGCAAAAACTGAAATACCATACTGACGATGTGTTCCAAAATA
GATATTCGTCTGAGTCATCAAACGGGGTTATTAAAATATCATCTACACCTGCAGCTTTAG
CTGGAGTTGATGATACTACGGCAATGACTCCACTGAAAACACAAAAACTTGCAATAAAAT
TAATTTCACAAATAGCTCCTTCCGAAGATACTGCATCAGAATCTGTGAGAGGTGTAGTTC
AATTATCTACTGTTGCGCAAACACGTCAAGGAACTCTCCGCGAAGGATATGCAATTTCTC
CGTATACATTTATGAATTCTGTTGCAACACAAGAATATAAGGGCGTTATACGTTTAGGAA
CACAATCGGAAATTAACAGTAATTTAGGAGATGTTGCAGTAACAGGTGAAACACTAAATG
GTCGAGGAGCTACCGGTTCTATGCGTGGGGTAGTAAAATTAACGACGCAAGCTGGTATTG
CTCCTGAAGGTGATAGCTCTGGGGCATTAGCATGGAACGCAGATGTAATTAATACTCGCG
GTGGACAAACTATTAATGGTTCTTTAAATTTAGACCATCTTACAGCAAACGGAATTTGGT
CGCGCGGCGGGATGTGGAAAAATGGCGATCAACCTGTTGCCACTGAAAGATATGCGTCAG
AAAGGGTTCCGGTTGGAACTATTATGATGTTTGCTGGAGATTCAGCTCCTCCTGGTTGGA
TTATGTGCCATGGCGGAACTGTGTCAGGAGATCAATATCCTGATTATAGAAACACAGTTG
GAACGAGATTTGGTGGCGATTGGAATAATCCTGGCATTCCTGATATGCGAGGTCTTTTCG
TCAGAGGAGCTGGTACTGGTGGTCATATTTTAAATCAACGAGGGCAAGATGGTTACGGGA
AGGATAGACTTGGTGTAGGATGTGACGGAATGCATGTTGGCGGTGTTCAAGCACAACAGA
TGTCATACCATAAACATGCTGGCGGTTGGGGAGAATACCAAAGACATGAAGCTCCATTTG
GCGCGTCCGTTTATCAAGGATATCTTGGAACTAGAAAATATGCCGACTGGGATAATGCTT
CATACTTCACTAATGATGGATTTGAATTAGGTGGACCGAGAGATGCCCTTGGTACACTTA
ATCGTGAAGGATTAATTGGTTATGAAACCAGACCATGGAATATATCATTAAACTATATTA
TTAAAATTCATTACTAAGGATTACAAATGATTGAATTAAAAGATTTACCTTTTGTTGATA
GCGTTCCTGGCGAAGGTCAGGAACGTATTTCATGGATTAAAAATGGTGAAGAAATACTAG
GAGCTAGTACTAAGTATGGAAACGATGGATCAATGAATAGACCAACTGTTTCCGTGTTTA
AAAATGTTGAGGTTCTTGACGAAAATATAGGAATTCTTAAAACAGCATTAGAAACATCCC
AGGAAGATATTAAAACCATTCAGGGTATCTTAGATGTATCTGGTGATATAGAAGCTTTAT
CGCAAATAAGTATCAATAAAAAGGATATCACTGATCTTAAAACGCTTACGAGTGAACATA
CCGAAATATTAAATGGAACTAATAATACTGTCGATAGCATTCTTGCTGACATTGGTCCAT
TTAATTCGCAAGCAAATTCAGTTTATAGAACGATTAGAAATGATTTGTTATGGATTAAAA
GGGAATTAGGACAATATGCTGGGCAAGACATTAACGGTCTTCCTGTTGTAGGAAATAGCA
GCACGGGTATGAAGCACCGTATCATTAATAATACAGATGCTATAACTTCCCAAGGAATAC
GTCTAAGTGATTTGGAAGAAAAATTTATTGAATCTGATGTAGGTTCACTGACAATTGAAG
TCGGAAATCTTAGACAAGAACTTGGACAAAGACCATCTTCATTTAGTGAAAGCATATATT
CACGTGTGCATAATTTAGATCAATCACAGATTAATCTTAGTTCCGAAATTAAATCTATCA
AAACTTCTATTGGATATCCAGGAAATACTTCAATAATCACAGATGTTAACACTAATACAC
GAGAAATTTCAGCTTTAAATGATGAACTAAATAAAAGCGGAGGTATTAAACAACGATTGA
CTGTCATTGAAACATCAATTGGCTCAGATGATATTCCTTCTAGTATTAAAGGTCAAATTA
AAGATAATACCACATCAATTGAGTTTTTAAATGGAATTGTTGGTGAAAACACTTCATCTG
GACTACGAGCAAATGTTTCATGGCTAAACCAAATCGTTGGAACTGATTCTAGTGGAGGTC
AGCCTTCTCCTCCTGGGTCTCTTTTAAATAGGGTTTCAACTATTGAAACCTCTGTTAATA
GACTTAACAATGATGTTCAAAACCTGCAAGTTGAAATTGGTAATAACACTACTGGAATTA
AAGGACAGGTTGTTGCACTAAATAAACTAGTAAGCGGCACAAATCCAAATGGTTCAACGG
TAGAAGAGCGTGGATTAACTAATTCAATAAAAGCTAATGAAACTAACATTTCTTCTATTA
ATCAAGAAATGCGTAGTATTAAAAGTAATGTATCTTCATTACAGGGGGATGTTCAGACTT
TACAGGAAGCAGGTTATATCCCTGAAGCTCCAAAAGATGGCCAAGCTTACGTTCGTAAAG
ACGGCGAATGGGTACTACTTTCTACCTTTTTATCACCAGCATAACATGGGGCCGCAAGGC
CCCAAAGGATTTTAAATGTCAGGATATAATTCTCAGAATCCAAAAGAACTCAAAGATGTC
ATTCTAAGACGTTTAGGGGCTCCAATTATCAATGTTGAGTTAACACCCGATCAAATTTAT
GATTGCATCCAGCGTGCCCTAGAATTATACGGTGAATACCATTTTGATGGACTTAACAAA
GGTTTTCATGTATTTTATGTAGGAGATGACGAAGAAAAATATAAAACCGGAGTCTTCGAT
TTAAGAGGTTCAAATGTATTCGCAGTAACTCGCATTCTCCGCACGAATGTTGGATCAATA
ACGTCAATGGACGGAAATGCTACATATCCATGGTTTACTGACTTTCTTTTGGGAATGGCT
GGTATTAATGGTGGCATGGGGACTTCTTGTAATAGATTTTATGGACCAAATGCCTTTGGA
GCTGATTTAGGATATTTTACCCAGCTTACCAGTTACATGGGAATGATGCAAGATATGCTT
TCGCCTATTCCAGATTTTTGGTTTAATTCAGCAAATGAACAGCTAAAAGTCATGGGAAAC
TTCCAAAAATATGATTTAATTATCGTAGAAAGCTGGACTAAATCATATATTGATACTAAT
AAAATGGTTGGAAATACAGTAGGATATGGAACAGTCGGTCCACAAGATAGCTGGTCATTA
TCTGAACGATATAATAATCCAGACCATAATTTAGTAGGTCGTGTTGTCGGTCAAGACCCA
GATGTTAAGCAAGGTGCTTATAATAACCGTTGGGTGAAAGACTATGCTACAGCTTTAGCT
AAAGAACTGAATGGCCAAATTTTAGCACGTCACCAAGGTATGATGCTTCCTGGCGGTGTT
ACAATTGATGGACAGCGCTTAATAGAAGAAGCTCGATTGGAAAAAGAAGCACTACGCGAA
GAATTATACTTGCTTGACCCTCCGTTTGGAATTTTGGTAGGTTAATATGGCTACTTACGA
TAAAAATCTTTTTGCTAAATTAGAAAACCGCACAGGTTATTCTCAGACTAATGAAACTGA
AATACTAAATCCTTATGTAAATTTCAATCATTATAAAAACAGTCAAATATTAGCTGATGT
ATTAGTAGCTGAAAGCATTCAAATGCGAGGCGTAGAATGCTATTACGTTCCAAGAGAGTA
TGTATCTCCTGATTTGGTATTCGGTGAAGACCTAAAAAATAAATTTACTAAAGCTTGGAA
ATTTGCTGCATATTTAAATTCGTTTGAAGGATATGAAGGAGCTAAATCGTTCTTTAGTAA
TTTTGGTATGCAAGTACAGGATGAAGTAACTTTGTCCATTAATCCAAACCTGTTTAAACA
TCAAGTAAATGGAAAAGAACCCAAGGAAGGTGATTTGATATATTTTCCTATGGATAACAG
CTTATTTGAAATTAACTGGGTTGAACCATATGATCCATTTTATCAATTAGGCCAAAATGC
TATTCGTAAAATTACAGCAGGTAAATTCATTTACTCTGGAGAAGAAATTAATCCAGTTCT
GCAGAAAAATGAAGGAATTAACATTCCAGAATTTAGTGAATTAGAATTAAATCCTGTTCG
CAATCTTAACGGTATTCATGATATTAATATTGATCAGTATTCTGAAGTAGATCAAATTAA
TTCTGAAGCTAAAGAATATGTTGAACCTTATGTTGTTGTCAATAACAGAGGAAAATCTTT
CGAATCTAGCCCATTTGATAATGATTTCATGGATTAATAAATATTATAAACTAATTAAAG
CCCAGATTAGGAGAAATCATGTTTGGTTATTTTTATAATTCGTCTTTTAGACGATATGCT
ACCTTGATGGGCGATTTGTTTTCAAATATCCAAATCAAACGTCAGTTAGAATCTGGTGAT
AAGTTTATACGTGTTCCTATTACGTATGCATCAAAGGAACACTTTATGATGAAATTAAAT
AAATGGACATCAATAAATTCACAAGAAGATGTAGCTAAAGTTGAAACTATTCTACCTCGT
ATAAATTTACATTTAGTTGATTTTAGCTATAATGCTCCATTTAAAACAAACATTTTGAAT
CAAAATTTACTGCAAAAAGGCGCAACTTCTGTAGTATCACAGTATAATCCATCTCCTATT
AAAATGATTTATGAATTGAGTATATTTACTCGCTACGAAGACGATATGTTTCAAATAGTT
GAACAGATTCTTCCATATTTTCAACCTCATTTTAATACAACCATGTATGAGCAGTTTGGA
AATGATATTCCATTTAAAAGGGATATTAAAATTGTACTGATGTCTGCTGCTATAGACGAA
TCTATAGATGGAGATAATTTATCTCGTCGTAGAATTGAATGGTCACTAACATTTGAAGTA
AATGGATGGATGTATCCTCCAGTAGATGATGCAGAAGGATTAATTCGTACTACTTATACA
GATTTTCACGCCAATACAAGAGATTTGCCTGACGGTGAAGGCGTTTTTGAATCTGTCGAT
AGCGAAGTTGTTCCTCGAGATATTAACCCAGAAGACTGGGATGGAACAGTAAAACAAACT
TTCACTAGCAATGTAAATAGACCAACACCGCCAGAACCTCCTGGCCCAAGAACATAGAGG
TTATTATGGAAGGTCTTGATATAAACAAACTTTTAGATATTTCTGACCTCCCCGGAATCG
ACGGGGAGGAAATTAAAGTATATGAACCTCTGCAATTAGTAGAAGTTAAAAGCAATCCTC
AAAACCGTACTCCTGACTTAGAAGATGATTATGGAGTAGTTCGTCGAAATATGCATTTTC
AACAACAAATGCTAATGGACGCAGCCAAGATTTTTCTTGAGACGGCAAAGAATGCTGATT
CTCCTCGTCACATGGAAGTATTTGCAACTCTTATGGGGCAAATGACTACGACGAACAGAG
AAATACTGAAGCTTCATAAAGATATGAAAGATATTACATCTGAGCAGGTTGGCACTAAAG
GTGCTGTTCCTACAGGTCAAATGAATATTCAAAATGCGACAGTATTCATGGGTTCACCAA
CAGAATTAATGGACGAAATTGGTGATGCTTACGAGGCTCAAGAAGCTCGTGAGAAGGTGA
TAAATGGAACAACCGATTAATGCATTAAATGATTTCCATCCGTTAAATGAAGCTGGAAAA
ATTTTAATAAAACACCCAAGCTTAGCTGAAAGAAAAGATGAAGATGGAATTCATTGGATA
AAATCTCAGTGGGATGGAAAATGGTATCCTGAAAAATTCAGTGATTACCTTCGTCTGCAT
AAAATAGTAAAAATTCCAAATAATTCTGACAAGCCTGAATTATTTCAAACTTATAAAGAT
AAGAATAATAAAAGATCTCGGTATATGGGTCTTCCTAACTTGAAACGAGCTAATATTAAA
ACACAATGGACTCGTGAAATGGTAGAGGAGTGGAAAAAATGCCGAGACGATATTGTTTAT
TTTGCAGAAACATACTGTGCCATTACTCATATTGACTATGGTGTCATAAAGGTTCAATTA
CGTGACTATCAGCGTGATATGCTCAAAATAATGTCATCTAAACGTATGACTGTTTGTAAT
TTATCACGCCAGCTCGGTAAAACAACTGTAGTAGCTATTTTCCTTGCACACTTTGTGTGT
TTTAACAAGGATAAAGCTGTAGGTATTCTTGCTCACAAAGGCTCAATGTCTGCGGAAGTT
TTAGATCGTACTAAGCAAGCAATTGAACTGCTTCCTGACTTTTTACAGCCTGGTATAGTT
GAATGGAATAAAGGGTCTATTGAACTAGATAATGGTTCTTCAATTGGCGCTTATGCTTCC
TCTCCTGACGCAGTTCGTGGTAACTCGTTCGCAATGATTTACATTGACGAATGTGCATTT
ATTCCAAACTTCCATGATTCCTGGCTTGCTATTCAACCAGTAATTTCATCTGGTCGTCGT
TCGAAAATTATTATTACTACGACTCCTAATGGATTAAACCATTTTTACGATATTTGGACT
GCAGCTGTTGAAGGTAAATCAGGGTTTGAACCATATACTGCTATTTGGAATTCAGTTAAA
GAACGTCTTTATAATGATGAAGATATTTTTGACGATGGATGGCAATGGAGCATACAAACC
ATTAATGGTTCTTCATTAGCTCAATTCCGTCAAGAACATACTGCAGCGTTTGAAGGGACT
TCTGGTACATTAATTTCAGGAATGAAATTAGCTATTATGGATTTTATTGAAGTAACACCA
GATGATCATGGTTTTCATCGATTTAAAAACCCTGAACCAGATAGAAAATATATTGCAACT
CTAGACTGCTCAGAAGGTCGTGGGCAAGATTATCACGCTTTACATATTATTGATGTTACC
GATGATGTGTGGGAACAGGTTGGTGTTTTGCACTCAAACACTATTTCTCATTTAATTCTA
CCTGACATCGTTATGCGTTATTTAGTAGAATACAATGAATGCCCAGTTTATATTGAATTA
AATAGTACTGGTATGTCAGTTGCGAAATCACTTTATATGGATTTAGAATACGAAGGTGTT
ATCTGCGATTCATATACTGATTTAGGAATGAAACAAACTAAACGCACGAAAGCGGTAGGA
TGCTCTACATTAAAAGACCTTATTGAAAAAGATAAGCTTATTATTCATCACCGAGCGACT
ATTCAAGAATTTAGAACGTTTAGTGAAAAAGGCGTGTCTTGGGCGGCTGAAGAAGGTTAT
CATGATGATTTAGTAATGTCTTTAGTGATTTTTGGATGGTTATCAACGCAGTCAAAATTT
ATTGATTATGCGGATAAAGATGACATGCGATTAGCATCTGAAGTATTTTCAAAAGAGCTT
CAGGATATGAGCGACGACTACGCGCCAGTTATATTTGTGGATTCGGTTCATTCTGCTGAG
TATGTTCCAGTATCTCATGGTATGTCAATGGTATAAATATATTAAAGCATATTAAAGAGG
ATTAAAAATGACTTTATTATCTCCGGGCATTGAGCTCAAAGAAACTACGGTTCAAAGCAC
CGTGGTTAATAACTCTACTGGTACAGCAGCTTTGGCCGGTAAATTCCAGTGGGGTCCTGC
TTTTCAGATTAAACAGGTTACAAATGAAGTAGATTTAGTTAATACTTTTGGTCAACCAAC
CGCTGAAACTGCTGACTATTTTATGTCTGCGATGAATTTCTTGCAGTACGGAAATGACTT
ACGAGTAGTTCGTGCTGTTGATAGAGATACCGCTAAAAACTCATCGCCAATTGCTGGTAA
TATTGAATACACAATTTCTACCCCAGGTAGTAACTACGCGGTTGGAGATAAAATCACGGT
CAAATATGTTTCAGATGATATTGAAACTGAAGGTAAAATTACTGAAGTAGACGCAGATGG
AAAAATTAAGAAAATTAATATTCCTACTGCAAAAATTATCGCTAAAGCGAAAGAAGTAGG
CGAATATCCAACACTAGGTTCTAACTGGACTGCGGAAATTTCTTCGTCTTCCTCTGGTTT
AGCTGCAGTAATAACTCTTGGAAAAATTATTACTGATTCTGGTATTTTATTAGCTGAAAT
TGAAAATGCTGAAGCTGCTATGACAGCGGTTGACTTTCAAGCAAATCTCGAAAAATACGG
AATTCCAGGAGTAGTAGCTCTTTATCCAGGCGAATTAGGCGATAAAATTGAAATTGAAAT
CGTATCTAAAGCTGACTATGCAAAAGGAGCTTCTGCATTACTCCCAATTTATCCGGGCGG
TGGTACTCGTGCATCTACTGCTAAAGCAGTGTTTGGATATGGACCACAAACTGATTCACA
ATACGCTATTATAGTTCGTCGTAATGATGCTATTGTTCAAAGCGTTGTTCTTTCAACTAA
GCGTGGTGAAAAAGATATTTACGATAGTAACATCTATATCGATGACTTTTTCGCAAAAGG
TGGCTCAGAATATATTTTTGCAACTGCACAAAACTGGCCAGAAGGCTTCTCTGGAATTTT
AACTCTGTCTGGTGGATTATCATCAAATGCTGAAGTAACAGCAGGAGATTTGATGGAAGC
TTGGGACTTCTTTGCTGACCGTGAATCTGTTGATGTTCAGTTGTTTATTGCGGGTTCTTG
TGCCGGTGAATCTTTAGAAACAGCATCTACTGTCCAAAAACACGTCGTTTCAATTGGGGA
TGCTCGCCAAGATTGCTTAGTATTGTGCTCTCCTCCGCGTGAAACTGTAGTTGGAATTCC
TGTAACTCGTGCTGTTGATAACTTAGTCAATTGGAGAACTGCAACAGGTTCATACACTGA
TAATAACTTTAATATCAGTTCAACCTATGCGGCAATTGATGGTAACTATAAGTATCAGTA
TGACAAATATAATGATGTGAATCGTTGGGTTCCATTAGCAGCTGATATTGCTGGTTTATG
CGCAAGAACTGATAACGTTTCTCATACTTGGATGTCTCCAGCTGGTTATAATCGTGGCCA
AATTTTGAACGTTATTAAACTGGCTATTGAAACTCGCCAAGCTCAGCGCGACCGTTTATA
CCAAGAAGCTATCAACCCGGTAACTGGTACAGGTGGTGATGGTTACGTATTGTATGGTGA
TAAAACAGCTACTTCTGTTCCTTCTCCATTTGATCGTATTAACGTTCGTCGTCTGTTTAA
TATGTTGAAAACGAATATCGGACGTAGTTCAAAATATCGTTTGTTCGAATTAAACAACGC
GTTTACTCGTTCATCATTCCGCACAGAAACTGCCCAGTACTTACAGGGAATTAAAGCTCT
CGGTGGAATTTATGAATATCGCGTAGTTTGCGATACAACAAATAACACTCCGTCAGTAAT
TGATAGAAATGAGTTTGTTGCAACATTCTACATCCAACCGGCGCGCAGTATAAATTATAT
TACTTTGAATTTCGTCGCAACTGCTACCGGCGCAGATTTCGATGAGTTAACTGGTCTTGC
AGGTTAATACGGTGCATTCTAAAGGCCTGTTTCGGCAGGCCATATAAATACACTATATCC
TTAATTCTTTAATTCTATATGCCCTAGGTTAAACATAGGGATATAAATACTACAGAGGCT
AATATGTTTGTAGATGATGTAACACGAGCGTTTGAATCTGGTGATTTTGCTCGACCTAAC
TTATTCCAAGTAGAAATTTCTTATCTTGGACAAAATTTTACGTTTCAATGTAAAGCCACT
GCTTTACCAGCTGGTATTGTAGAAAAAATTCCAGTCGGATTTATGAACCGTAAAATTAAC
GTAGCAGGCGATCGTACATTCGATGATTGGACTATTACAGTAATGAATGATGAGGCGCAT
GATGCACGCCAGAAGTTTGTTGACTGGCAAAGCATTGCTGCTGGGCAAGGAAACGAAATT
ACTGGTGGAAAACCGGCAGAGTATAAAAAGAGTGCTATTGTACGCCAATACGCTCGTGAC
GCTAAAACTGTAACAAAAGAAGTCGAAATTAAAGGCCTGTGGCCTACTAACGTGGGTGAA
CTTCAATTAGATTGGGATTCAAACAATGAAATCCAAACCTTTGAAGTAACTCTCGCTCTC
GATTATTGGGAATAAAATGAATGGGGAGAAATCCCCATCCTGCTTAAAGCAGAGAAGTCC
ATTATAAATATAACTATAATTCCCATTTGGAGAATACAATGAAATTTAATGTATTAAGTT
TGTTTGCTCCATGGGCTAAAATGGACGAACGAAATTTTAAAGACCAAGAAAAAGAAGATC
TTGTTTCCATTACAGCCCCAAAGCTTGATGATGGAGCAAGAGAATTTGAAGTAAGCTCGA
ATGAAGCTGCTTCTCCTTATAATGCTGCATTCCAAACAATTTTTGGTTCATATGAACCAG
GAATGAAAACCACTCGTGAGCTTATTGATACATATCGTAATCTCATGAATAACTATGAAG
TAGATAATGCAGTTTCAGAAATTGTTTCAGACGCTATCGTCTATGAAGATGATACTGAAG
TCGTTGCGTTAAATTTGGATAAATCTAAATTTAGCCCAAAAATTAAAAATATGATGTTAG
ATGAATTTAATGATGTATTAAATCATTTATCATTTCAACGAAAAGGTTCTGATCATTTTA
GACGTTGGTATGTCGATTCAAGAATTTTCTTTCATAAAATTATTGATCCAAAACGTCCAA
AAGAAGGCATAAAGGAATTACGTAGATTAGACCCTCGCCAAGTTCAGTATGTTCGTGAAA
TTATAACAGAAACTGAAGCTGGCACAAAAATAGTTAAAGGTTACAAAGAGTATTTTATAT
ATGATACTGCGCACGAATCATATGCGTGTGATGGTAGAATGTATGAGGCTGGCACAAAAA
TAAAAATTCCTAAAGCTGCCATCGTTTATGCCCATTCTGGATTAGTCGATTGTTGCGGTA
AAAATATCATCGGGTATTTGCATCGTGCTGTTAAACCTGCTAACCAATTAAAATTATTAG
AAGATGCTGTAGTCATTTATCGCATTACTCGTGCTCCTGACCGTCGTGTATGGTACGTAG
ACACAGGTAATATGCCTGCTCGTAAAGCTGCCGAGCACATGCAACATGTCATGAACACGA
TGAAAAACCGTGTAGTATATGATGCATCAACAGGTAAAATAAAAAATCAACAGCATAATA
TGTCTATGACCGAAGACTATTGGTTGCAGCGCCGTGATGGTAAAGCTGTGACAGAAGTTG
ATACTCTTCCTGGCGCTGATAATACTGGTAATATGGAAGATGTTCGTTGGTTTAGACAAG
CTCTTTATATGGCATTACGTGTTCCTCTTTCACGCATTCCGCAAGACCAACAAGGCGGTG
TGATGTTTGATTCTGGAACTAGCATTACACGCGATGAATTGACGTTTGCTAAATTTATTC
GTGAGTTACAGCACAAGTTTGAAGAAGTTTTCCTAGATCCGCTTAAAACAAATCTTTTGC
TTAAAGGTATAATCACAGAAGATGAGTGGAATGATGAAATAAATAATATTAAGATAGAAT
TTCATCGGGATAGCTACTTTACTGAACTCAAAGAAGCAGAAATTTTGGAACGAAGAATTA
ATATGCTAACCATGGCAGAACCATTTATTGGTAAATATATTTCTCACAGAACTGCTATGA
AAGATATTTTGCAGATGACTGATGAAGAAATAGAACAAGAAGCCAAGCAAATTGAAGAAG
AGTCTAAAGAGGCTCGTTTCCAAGACCCCGACCAAGAACAAGAGGATTTTTAATGGAAGG
TTTAATTGAAGCTATTAAATCAAACGACCTCGTAGCCGCTCGTAAATTATTTGCTGAAGC
CATGGCTGCAAGAACGATTGATTTAATTAAAGAAGAAAAAATCGCTATCGCTCGTAATTT
CTTAATTGAAGGTGAAGAACCTGAAGACGAGGATGAAGATGAAGATGATGACGAAGATAG
CGATGATAAAGACGACAAAAAAGACAAAGACTCTGACGAAGACGAGGATGATGAATAATG
CTTCTGATCCCTGAAACTCATGAATTAGTTCTCGAGAATGTCGAAGCACTTATTCCTGAA
GCACAGGGTCGCTTTGACGAATTGTCTTCTGCTTTAAATAAAGACGATATAAATACAATT
GTCGAGAATATGCTTGATGATGAAACTGATTTAGCGGTTGCATTAGCTTCTATTAATGAA
AATATGCCGTTAAATGAATTCATCGTTAAACATGTTTCTGCCCGTGGTGAAATTACTCGC
ACTAAAGATCGCAAAACCCGCGAACGAAATGCATTTCAAACCACTGGGCTGTCTAAAGCA
AAACGTAGACAAATTGCTCGTAAAGCTACCAAAACGAAGATTGCCAATCCAGCAGGTCAA
TCTCGTGCTCAGCGTAAGCGTAAAAAAGCTCTTAAACGCCGTAAAGCATTAGGATTAAGC
TAATGAATGAACCCCAATTACTAATTGAAACTTGGGGTCAACCTGGCGAAATTATTGATG
GCGTGCCAATGCTTGAATCTCACGATGGAAAAGACTTAGGTTTAAAACCGGGTTTATACA
TCGAAGGAATATTCATGCAAGCGGAAGTCGTCAATAGAAATAAACGTCTTTATCCAAAAC
GTATATTAGAAAAAGCGGTAAAAGACTATATTAATGAGCAAGTTTTAACTAAACAAGCTC
TAGGAGAATTAAATCATCCTCCACGCGCTAATGTTGACCCGATGCAAGCCGCTATCATTA
TAGAAGATATGTGGTGGAAAGGAAATGACGTATACGGACGAGCTCGTGTTATTGAAGGTG
ACCACGGTCCTGGAGATAAATTAGCAGCTAATATTCGTGCCGGATGGATTCCAGGAGTTT
CTTCTCGTGGATTAGGTTCATTGACTGACACAAATAAAGGTTATCGTATCGTAAACGAAG
GATTCAAATTAACTGTAGGTGTTGATGCAGTATGGGGTCCAAGTGCTCCAGATGCATGGG
TAACTCCTAAGGAAATTACCGAATCACAGACGGCGGAAGCCGATACAAGTGCCGATGACG
CCTATATGGCTCTCGCAGAGGCCATGAAAAAAGCGTTATAAATATTATTATCTAAACAAC
AGGACTACAAAATGCTTAAAGAACAACTGATTGCTGAAGCGCAAAAAATTGATGCTTCCG
TTGCTCTTGATAGTATTTTCGAATCAGTTAATATTTCTCCGGAAGCAAAAGAAACTTTCG
GCACTGTATTCGAAGCTACCGTCAAGCAGCACGCCGTTAAATTAGCTGAATCTCATATCG
CTAAAATTGCTGAAAAAGCAGAAGAAGAAGTAGAAAAAAATAAAGAAGAAGCTGAAGAAA
AAGCTGAGAAGAAAATCGCTGAGCAAGCTTCTAAATTCATCGACCATCTTGCGAAAGAAT
GGCTCGCTGAAAATAAATTAGCAGTTGATAAAGGCATCAAAGCCGAACTGTTTGAATCCA
TGCTTGGTGGATTAAAAGAGCTCTTTGTTGAACACAACGTTGTTGTTCCAGAAGAATCAG
TTGATGTTGTAGCTGAAATGGAAGAAGAGCTGCAAGAACATAAAGAAGAATCAGCTCGTC
TGTTCGAAGAACTCAATATGCGCGACGCATATATCAATTATGTGCAGCGTGAAGTGGCAT
TGAGCGAAAGTACTAAAGATCTGACTGAGTCTCAAAAAGAAAAAGTCTCTGCTCTGGTCG
AAGGTATGGATTATTCAGATGAATTCTCAAGTAAATTGAGTGCCATCGTAGAAATGGTGA
AGAAGTCTAATAAAGATGAAAGCACTATTACTGAGGGTATAAATACTCCTGATACTGAAG
CAGCCGGACTGAATTTCGTCACTGAAGCTGTAGAAGATAAATCTGCATCTGGTGCAGAAG
ATATTGTAAGTGTATATGCGAAAGTCGCATCTCGTTTCTAATTTTAAAGGTTAACACAAA
TGACTATCAAAACTAAAGCTGAACTTTTGAACAAATGGAAGCCATTACTGGAAGGTGAAG
GTTTACCGGAAATTGCTAATAGCAAACAAGCGATTATCGCTAAAATCTTTGAAAACCAGG
AAAAAGATTTCCAGACAGCTCCGGAATATAAAGACGAAAAAATTGCCCAGGCATTCGGTT
CTTTCTTAACAGAAGCTGAAATCGGTGGTGACCACGGTTACAATGCTACCAACATCGCTG
CAGGTCAGACTTCTGGCGCAGTAACTCAGATTGGCCCAGCTGTTATGGGTATGGTACGTC
GTGCTATTCCTAACCTGATTGCTTTCGATATTTGCGGTGTTCAACCGATGAGCAGCCCAA
CCGGTCAGGTATTCGCACTCCGCGCGGTATATGGTAAAGATCCGATCGCTGCCGGTGCTA
AAGAAGCATTCCACCCAATGTATGGTCCAGATGCAATGTTCTCTGGTCAGGGTGCTGCTA
AGAAATTCCCAGCTCTGGCTGCTAGCACAGAAACCACAGTAGGTGATATCTATACTCACT
TCTTCCAGGACACTGGTACTGTGTATCTGCAAGCTTCTGCTCAAGTAACAATTAGTGCAA
GTGCAACTGATGCAGATAAATTAGATGCTGAAATTAAGAAACAAATGGAAGCTGGTGCAC
TGGTAGAAATCGCTGAAGGTATGGCTACTTCTATCGCTGAACTCCAGGAAGGTTTCAACG
GTTCTACCGATAACCCATGGAATGAAATGGGCTTCCGTATCGATAAACAAGTTATCGAAG
CTAAATCTCGCCAGCTGAAAGCTGCTTACTCTATCGAATTAGCACAAGACCTCCGTGCTG
TTCACGGTATGGATGCTGATGCTGAACTGTCTGGTATTCTGGCTACAGAAATCATGCTGG
AAATCAACCGCGAAGTTGTTGATTGGATTAACTACTCAGCTCAGGTTGGTAAATCTGGTA
TGACCCTGACTCCGGGTTCTAAAGCTGGTGTATTTGACTTCCAGGACCCAATCGATATTC
GTGGTGCTCGCTGGGCAGGTGAATCCTTTAAAGCTCTGTTGTTCCAGATTGACAAAGAAG
CAGTTGAAATTGCTCGTCAGACTGGTCGTGGTGAAGGTAACTTCATTATCGCTTCCCGTA
ACGTAGTTAACGTTCTGGCTTCAGTTGATACCGGCATTTCTTATGCTGCACAGGGTCTGG
CTACCGGCTTTAACACTGATACTACCAAGTCAGTATTTGCTGGTGTTCTGGGTGGTAAAT
ACCGTGTATATATCGACCAGTATGCTAAACAGGATTATTTCACTGTAGGTTATAAAGGTC
CGAACGAAATGGATGCTGGTATTTACTACGCTCCATATGTAGCTCTGACTCCATTGCGTG
GTTCTGATCCGAAGAACTTCCAACCAGTAATGGGATTCAAAACTCGTTACGGTATCGGTA
TCAACCCATTTGCAGAATCCGCTGCTCAGGCTCCGGCTTCTCGCATTCAGAGTGGTATGC
CTTCTATCCTGAACAGCCTTGGTAAAAACGCTTACTTTAGACGTGTATATGTTAAAGGTA
TCTAAGATATAACCTTATTATGGGAGACTTCGGTCTCCCATTCTTGTTTCTATACTACGC
AATCTTAACTCGACGGTTGATGATGTCACGGAAGTCAATACGATTTTCTTGGATTTTCAG
CCAGTTACCAGATTTGTCTTTGGTATGGGTAGAATATTCACCGTTCCACCAGAAGTCAGT
AGCATAAGCCATGGTTTCATATTTCTTAACTAGGGCATTCACTGCATTACCGTTAGGATT
GGAACCATATTTTTCTACCAGGGCTTTGATGTCTTTATCAAGAGCGGACAGAGTAGTACG
AATAAAGTTTAACTTGTTCATTTTTAATCTCCTCATGTTTTTGATAGGTCTATAGTAACA
TGTGATAACCTTGCTGTAAACAACTTTTATAAATAAACATATACTCTCAACAAGGAAAGC
GCAATGGCTAAAATCAACGAACTTCTGCGCGAATCAACCACAACGAATAGCAACTCAATC
GGTCGCCCAAATCTCGTTGCTTTGACTCGCGCTACCACTAAATTAATATATTCTGACATT
GTAGCAACGCAAAGAACTAATCAACCTGTTGCTGCTTTTTATGGTATCAAATACCTTAAT
CCAGATAACGAATTTACTTTTAAAACTGGTGCTACTTACGCTGGCGAAGCTGGATATGTA
GACCGAGAACAAATCACAGAATTAACAGAAGAGTCTAAATTAACTCTCAATAAAGGCGAT
TTATTCAAATATAATAATATCGTTTATAAAGTATTAGAAGATACACCATTTGCTACCATT
GAAGAAAGCGATTTAGAATTAGCTCTTCAGATTGCAATTGTTCTTTTAAAGGTTCGTCTA
TTTTCTGACGCAGCGTCAACGAGCAAATTTGAAAGCTCTGATAGCGAAATTGCGGATGCT
AGATTCCAGATTAATAAATGGCAAACCGCAGTTAAATCTCGTAAACTTAAAACTGGCATC
ACAGTTGAATTAGCACAAGATTTAGAAGCAAATGGATTCGATGCTCCTAATTTCTTGGAA
GATTTGCTTGCAACTGAAATGGCGGATGAAATCAATAAAGATATTCTACAGTCTTTAATT
ACTGTATCGAAACGTTATAAAGTTACTGGAATTACTGACACCGGATTTATTGACTTGAGC
TATGCATCCGCTCCAGAAGCAGGTCGTTCATTATATCGAATGGTGTGTGAAATGGTTTCG
CATATCCAAAAAGAATCAACTTATACAGCAACGTTCTGTGTTGCTTCAGCTCGTGCTGCT
GCGATTCTTGCTGCATCAGGTTGGTTAAAACATAAACCAGAAGATGATAAATATCTTTCA
CAAAATGCCTACGGGTTCTTAGCTAATGGTTTACCGCTTTATTGCGATACTAACAGCCCA
TTAGATTATGTAATCGTTGGCGTGGTAGAAAATATTGGCGAAAAAGAAATTGTTGGATCA
ATTTTCTATGCTCCATACACAGAAGGTCTTGACTTAGATGACCCTGAACATGTTGGTGCA
TTTAAAGTTGTAGTTGATCCGGAAAGCTTGCAACCATCTATCGGTTTATTAGTTAGATAT
GCTTTATCAGCAAATCCTTATACTGTAGCAAAAGATGAAAAAGAAGCAAGAATAATTGAT
GGTGGAGACATGGATAAAATGGCGGGTCGTTCAGATTTGTCTGTTTTATTAGGTGTTAAG
CTACCAAAAATTATTGTTGATGAATAAAACAAAGGGACCGAAAGGTCCCTTTTTATTTAG
CTCACCAACTCAATCCAGGCTGGACGAAGTACATCTTGTACCATTTTAACTAATTCCTTT
TTAATCAAAGAAGGATTATCTGCTTGAGTCAGAGTAATACCTTCACGAGAAGTTTCTTCC
AAAATATCTTGAACAGTTAGTCCCATCACCTTTCCAAAATCTTTAGGACCAATTTCACCA
ATTTTAGAGATAACGTTATTTACTCGGTTCAGTGTAACGTAGCAAGCTAAAATTCTCAGC
AATTTATTATCAGCTTCTGACAACTCAACTTTAGCTTTAATAGGCTTATCAGACTTTTTC
TTTTCGCTAAATTTAGAGTTCTTGCATTTGATGGCTACACGATTGCCATTAGGCATCCAA
GAAGGATAACAAGGTTTCAATACATATCCTTCAGCGGTAAATACTTCGCCTTTTGCTTCG
GCATTCCAAACGCATTTATTTGCATCAGCTAATCCAGCATGGTCTACTGTAAAATTATAA
TCTTGGACGACAGAATCTAAATCATTTGGCAATTTAATAAGCTCTTCAAATTTACCGCGA
CCTAAAAGTGGAGCCATTTTAAATTTGAATGTATTACAGAATGATTCCATCATATAATCA
TCGACATAAGTCACGTCACCACTTTCTGTAGTGACAATAATGTCAAATACATAAAAATCT
TTATCGCCATAATCAACATTCTTCTGAATGCCAGGTCCAGCGAATTCGCCAAAGACTTGA
TAAGATACAACTGCTGAAGTTTCCATAATATCTTGCACTGCTTTAATAGAATCAGCATAA
TTCTTTAAAATAATTTCATACCCAAAGAAATCTTCAGCAGGAAGAATAGGACCAGTACGT
TTAGCACAAGTTACTTTATCACGCTCAATAATCAATGAGAAATTTGTACCATGAATCTTT
TCACGAGCTACCCACTCCCCACCAGTCAATCCCAAGCTATAAAGTTTTTCAATAAATTTA
GAGTTGTAATGATTTTCAAGACTGCTATACTTTTTAAACATAATTAATCCTCAAAATGTA
ATTTCTAACCAATCACCATCACGCTGGTCACTATTGGCTTTAAAGCTAAATCCTTCTTTT
CTCAGCCAATCACCAATTTCTTCAGTAATCAATTTATCGCGAGCAATGCAATAATAACTA
AAATATGTTTTACCTTGTTCAGCTGCTTTATTAGCAAGTTCTGAAAAATCTTTAATAAAA
CATTCTAGTTTAAACTGTTTACTTTTAAGTGCTTTTTCTCGCAATTGATTAGCAAAAGAT
TCATTTTCATAAAGATCATACTGTTCCATTTTTCACCTTTTTATTGATATGTCTTTTTCT
ATAGACAACTTTTTCTCGAGTTTCAAATTTATTCTTAGCAGTCATAGCTCGAGCCCATAA
TACAGCCACTTCTTTTGCCTGTAAGTTTAATTCACGAGCAATTTCAATGAATGACTTTCC
AGACTCATGAAGAGTAAACACCACAACCTCAGTTCTCATAATCAATCTCATGTTATCGAG
TTGGTGCCATTATATACATCATTTTCTGATTGTGTTTTGCGTGCTTTCAAAATGAAGAAA
GGGGGCCGAAGCCCCTTACGATTATGGGTATGTATAGATGATACCAGTTTCCAAAGCAGT
TTTATGGATGATGTATCCATTACGTGATTCCTGAACATCAACTTCCGGATAGTCTTTCAT
CATCTTCTGAAGAGTGTAACGATGCAGGTAGTATTTACTATCTGGATCTTCAGTTTTCCA
ATCTTTACCTTCTTCAGTCATTTTTTGGATTTCATCAATAACCCACCAACCGCACCAAAT
GTAAGCTGAAGTACGATGTGGAAGAGGATGAACATAAGGTAGTTCACCTTCTGGTTCTGG
AGCAACTTTATCAGTCACTTGAACTTGACCTTCTGCTGTAATAGTTTTGCTATCGTAATC
AGTTGCAGTAATAGTTGCAGTAACTTCAATAGTTTGACTTCCAATAGATGAGGTATCAAC
GGTATATACATTAGTTGACCCTTCTACAGGAGAAGAATCTTTCTTCCATGAATACACAAT
TTGCGCTTCTTCTGGAGCATCAGTGACATTAGCGGTAAATGTAGCCGAAGCATCTTGTTG
AACATTAATAGAAGGAGGAGTCAATGTAACCTGTGGATTCATTGTCTTCTTATTAACTGT
TAATGATACTTCATTCGAAGTAACTTCCTTTTCATTGTAATTTTCAGCAGTTACTTGGGC
TACGCATTTAATTCTTTTTACTCCACTTGTAGTTGGAGTATAGCTAAATGTAGAGTTAGT
TTCTCCACCAACTTGTGAATCATCTACATACCACTGATACGTAGCAGATGCTCCATCAGG
TTGAGAAGCTAAGGCAGCAGTAAATTGAACTGGGGTTCCAATTACTCCAGCTGAAGGACT
ATCGGGAGTTACTGCCAAAGTAGTTGTCTGTGTCTTTTTATTAACAGTCAGTGATACTTC
ATTAGAAGTAACTTCTTTTGCGTCATAATCCGTTGCCGTCACTTGGGCTACACACTCAAT
TGTTTTAACTCCGCTTGTGTCAGGAGTGTAATTGAATGTAGCGCTAGTCGCTTCGCTCAC
AGGAGAACCGTCAACATACCACTGATACGTAGCAGATGCGCCTGATGGCTGCGAAGCCAA
AGCAGCGGTAAATTCAACAGCTGTTCCAATTACTCCAGCCGCAGGACTAGCAGGAGTTAC
GACTAAGGTAGTCGTCTGTGTCTTATTTTTAACTGTGATAGTTGTTGTCGCTTCAGCCGT
TTCCGGACCTCCTTCAGAAAGTGTATTTGTTGCAACTACTTTAATAGTCTTTTGACCGGC
AGGTCCTTTTAATACATAATCAAAAGTTGCTTCAGCTCCATCTTGTGGAGCATCATCTAC
AGTCCAAGCATATGTAATAGTTCCGTCTCCAGTTTGACCACTGGGTGTAGCAGTAAACTG
CTTAGTTTCATCAATAACCCCTGTCGGTGTTTTAGGAGTTATATCAACTGTAAAAGCCAT
AAGTTATCCTTATTTTAATGTTACGAAAGAAGAATTGCGTGTTTCACGAATTAGAACTGA
TCCGTCGCGGTTAATGTAATAAATTAAGCTAAATAAAGTTTGGTGTGCTGACGCATGTTC
AAAACTTGTTGGGTGAGATTTCCAATCTGGAGTTTCAGCAATCCATTGATAAATCCACCA
AGGAACAGTGCAGAATCCTGGATTTTTTCCAATCAGCTGAAGATTTGGGCTAAAGTTTTC
TGGAAGAGTAAATACAGACGGCTTTTCAGATTCAATAATCTCAGCTACAGCCTGTTCGAA
TTTTTCTTCAACAAAAGGAGTGTCTTCAATCAAAACATCGGTATTTTCAGGAATTTTATC
CGTTTCTACTACTTCAATTTTAATGTCAGATTTAACCGGAGAATCAATCAAAAGTGCTGC
TTCCGGATTGACTTCTTCATCGTCATATTTTAATCCCTCTGCGGCATCAGCAGCATCAAT
TAAGTCTTTAATAGATAACCCATCAGTCTCTGGCATAGGTTCACTAGCGAGCTTCTGGAG
GGCTTCTTCAATATCAACAACGATATTATCAAAAGATTTATTCTTTTTGACCTTTATACC
AAACTGTTCAGCATATTCAGCTAATTTAGCTTTAGCTTCTTTGTTATCATCAAGAGCCTT
CAGCTCTGCAATATAAT

In another aspect, the invention provides an SFNHSI bacteriophage targeting a Shigella spp. bacteria. In certain embodiments, SFNHSI binds a molecule of OmpC. In certain embodiments, the SFNHSI bacteriophage has a genome comprising a nucleic acid sequence comprising SEQ ID NO: 8.

SFNHSI
(SEQ ID NO: 8)
TATTATTAAGTAAACTCATTATGCAATCCTTATCCAACGGTATACAGTAATAGACGGTTG
AATATTACTAATATTAGTAGGAGGAGTATGTGAAGAGTTTGTTGTTGCGTAGTCTTCACG
ATATTTTGTATATATAGGACCAGTTTCGTCTGGGTCATATTGACATCCTCCAATAATAAC
TGATCCATTTTCGTCTTCAATTAAAACTCTTTCATCAGTTTTAGTCGCAGGAAGATTTGC
GTTTTCAAGCGTTACTGATGTTGTACCAACTGTTCCACCGGCAGTATGTGAAGGATTTCC
GCTAGAATCTAAATCATTATTGTTTAAAGCAAAGTTTGGATCCGTAACATCATCATTCCA
TCCTACTAAAACTTGTCCTTTACCAAATAATTTCCATGAACCAAATCCCATATAAGTCAC
CGGATTATTTGGGTTTATAGCATTTTCATAAATTGAACCAACAGGATAAATGCTGTCGAA
AATAGATGATATTGAATTATACGTTCTAGTATAAGAATCAACTTTTTCAACATTTGGCCA
ACCGATTTTATCAAAATCTGTTAAGGCTACATCCCCAGTAACTTCAGTAGATGATCCTTT
ACTAACATAACGGTCATCAGTTAATTCAATAATGTCATCTTTTTCTAAAAGAGTTCCTAA
ATCATTATTAAACCATGTTATAACGATTATATCACCTGATTCAAATTTTCTATCAAATAA
AAGACTGACAGGTTTTCCATCTTCATATTCTATAGAATAATCTGTGTTCGATTCTTTCCA
TTCACCACCAAGAGATATACATCCTGCTTGCGTATCTGAGTTAGCACCTTCACACAAAAA
CAGAGGATATCCTGCTGTTCCAGCTTGTTGTTGTAAAATTCCATTAAAACGAACTTCAAG
AGAATTAGGATTAATAGGTTCTCCAGGAATTAATCCAAATGCAGAAAATGGAATTGATTT
CATTGTTGATAAATCAGTAACGTAAATACTTCCTTCTAAAGACGTTTTTGACGTTAGTTT
TGAATCCAGTACTTTTATTTGGCGTCTTGTATATGAACTTCTCCATTGTGATACACCATC
CATAAATGTTTCAATTTGAACAGTGTCACCAATATTACAAGGCTGTCTTAACCTAATATT
AAATCCATCAAGAGGAATCAATTCTCCTTCATTTTCTCCGGGAGAGCCAAAGTCACTGTT
TTCACTAAATACATCGCCGTAATATAATTCATTACCACGGTGTTTTACTCGGATGTTATT
AACATTATAGCTAGTTCCGCGGAAAACATCTAAAAAGTCTGTTTGTCCTTGAACTTCGAC
TAAAAATTCTTTACGTGCTACATTACTAATATCTGAACTAATAATTTTGTCAATTTGTTT
GTTTTTGACATATTCCCAACGTCCTGGAGCGCAATAAACGAGCTCTAAATCACTAAATTG
AATATTAATTTCAACTGACGATGACGACCCTTTAATTGTATCCCCAGAAGCAGCTACTAA
AGTAACTGGATTAATGTTCCATGTTGCAAATACATCCCTAGCTCTAATTACTTTATTATA
ATCATTAACAGTTCCTTTAGGAAGTTGTAAAGTTACTCTTCCAGAAGAGGTATTAATAGC
GTATGATTTTCCCCACTTAGCTTTTAATGTTTGACCGGATGAAGCATTATAAGTTTTCCA
GGCACCGGCTGAATATGGAACATCTCCATCGCCAAGTTCATAATAAAGTTCATCAAAGTT
TTCATTTATTTTTATACCACCTTTACGCAGGTAGTCACCGGTACCATCATCAACAACATT
ACCGATATTAATATTTTGTTTCATTATTGAGCCACCCCGATTTTCTGCGTAGCGATAACT
TTAACTGCTGCTCTCATGCCAACGGTAGAAGAACTTATAGTCGCCGTTACATAGTTGGTT
TTAATACTAAATGCAATATTAGCAATCTCGTCTTCTTCTGTTTCATTTCCAACTCTCATG
ACAGCATACTCAGAAGAAATAACTTCTGAATTAACAGTATCCACAAGAATATTTATTTCT
GCTGTTTTAATTTTTCTTCCATCTACTGATTGGCACGTAACTAGCAATTTAGCCATATTG
TATTCAGTGCGATGAAATAGTGGAATATCAACTGATCCGGATGTAGAAATATTCCATGTA
CCTTCAGCTGGTGATTCCTTTTGTCCAAACATACTTTCAATAGAATAATTCCAAACCGAT
GTAGAATTATCAGATGAAATACAGCGTAAAGTTACTTTACTATATGGGCTAGTTACTACT
AAATTACCTGAAACACCTTTAATTGAATCAATAGCTTGAATCGTTAAAGGATTAGTAACT
GATATTGATCCATTAGAGTTAATGAATTCAACACAATCGCCGAGTTCGCCTCTTTCAATA
ATAACTTTAACACCTACAGTAGAGGTATCAATATCATGTCTAGTGCCAACTTTCACTGGA
GTTGCGTACTCTGTAATAGAGTGTTTTTGATAATACCCAGTAGCATGGATAATTTGACCA
TCTGCTCCAGTGCCATTTGCTACTGCCATTTTACGCTGATCGCCAAACGCATTATAAATT
GCGTTAAAATCACTATTAATTTTATTACCACCGTCGAATAAGATATCACCAGTAGAAGCG
TTACCAATTTCGCCGGTATCAATCAATTTCTTTGGTTCTTGAATGAACATAGCGGTTTCC
TTATGAGTTTATAGTATTTATAAAGAAAAAGGGAGCCCATGGGCTCCCTTAATTTAAAAT
GTAAACAGAATATTGATTTCTTCTGTCTGATCCATTGCCATGATAATAGGTGGCCTATTT
TCCATATAAATCATTTCGCCCGAATGCCTCATTAAATCTTCTGGGTCATAATAATCCTTT
TCAGCTTTAACGTTTGGGTCATTTGGATGAGCTTTAGCTTCAAGAGGATTCGTGATTATT
GATATTTGTCTAAATCCTTTATTTCCTGGCAATGCAGCATCAGGAAAATAAACTGAATCT
AAATATGCTTTAAAACGGATAGTATTTGCCTTAACCCGGTAAATTAATCCAAAATCATCT
TGCTGCCAAGTGAGATTATCTTCATACCCCCATCTAGTTGGATCTTCTTTTAATTCCTCA
GGCCAAGGAACTACGATATATTCATTTGTGCATCTATTTATAGATACATCGGGCGGAATC
TCAAAAAGATATTCCCAAACATATCCGTCTCCAGGTTCGATTGTTCCTTCCGCATCTCCT
CGACCTTCAGGAGGAGTCATTGACCTAACAGAAGGAGTCCATTTTCCGCCTAATTTAAGG
CATTCATCCTTATCAGTTAAAGATGCAATTGAACACATTCCAGTATCAGGAACATCTAAA
CAACGATATACTAACCAGCCTGCGCCTGATTCAGTAGCGTTGTAAGGAGCTGAGTTACAC
ACTACAATATCGTTAATTCTAAATGTGTATGGATCCGGATATCTAGTATCTCCCCAATCT
CGACGAGGAATAACTGCGTCAAGCATTGATGGAAGAACCTTTACTGTTCCCATCATATGC
GTCCACATGTCAGTTACGCCTAATACAGAATCGGTTGGATAAGGTGGGGCAAAGCCCACC
TCATTTTCATTTGATGACCACGGTTCTGATCTTCCAAATGTGATAAAGATAGTGTTTTTA
TCCGGACCACTTCCAATTGAATTATAAAAATTCAACATTTTTTCTGTTCTAAATTTTGAA
GTAACTATCGCACGATAGATAACACTTGAATCATTCATCTATTTTAACCTGTGTTGGATT
TTCAGGGTCTCTTGGATTTCCTATATTATCTTTTAGACGTTTATTAACTAAATCCCTAAA
TTGCGTAAATGTTGTTCCAGAAGCATCAAATAGGGGACTCATTAATTTACGTCGTTCAGA
CGGCAATTGACCTTGAAATATTGAATTATTATTTTCAGCATTATAGTCATCAGGAAGAGG
ATATTCTACACCAGCCATTGGGCCAGGCTCATAAATTGCTTCGCCTGTTACTGAATCATG
CTCAATTTCACCGGTTGGAGTTAATTTAGCTACTCTATCAGCATATTCAGTAGGCAATCC
AGAATCCCATTTATAGTTTTTATATTTATTAATTATTGTCTCTGTGTGTTTAAGAGTTAA
ACCAACATTAATAAACATCGTTAAAAGGGTAATTGCTATAAATCCAAATCCTACTGGATG
AACAAAACGAATAACGTCAGATTTCCAGCGGGAAGAAGGTAAATTGGATTTAATTTTCAT
TACATAGTATGATCTACTTCTATTAATATAGTCTATATTGTTTTGAAGCAAATCCTTTCC
TTTAACTCCACGAATAATTTCGCCTTCAAAACTAGGGAGTCTTTCTGCTTTAACTTCTTG
ACCAGCAATTAATCTTCCTAAAAGATTATGAATAGTTACGGTCCATTGCAATTTACCATT
AGAATAGCTTCTTTCTATATAAGTAACATTACATCTTCCTGTTGCCGTATAAATCGTTTG
TCCTACTAAATCTTCAGTCAAAGAATCAGATTGAACGATTATGTCATATTCAGTACCGGC
CCCAGATTCAATTTCAATTTCAACTTCTTCATTATAAAGAACTTTAAAAAGAAACTTGTA
TGATGCTTCAATTCCTTTAGTAGAATAAAAATCATAGCTACGTGATTCAAAGAATCTCGC
AACAGCATCACGTTTATCAGCATTTAAATAAATGTTTCTTTTATATATCTCTGACCACAA
ATATTCCCACGCATGCTCTTCTCGTGGATATTGGTTACGAATTAAATTTCGTAAATTGTT
ATACTGAGTTCCATATCCGTCAGAAAGATATTGAATATATGCTTCACAAAATGCCTCAAA
ATTAGAATCTTGTAACAAGTAAGAATCAGGCATCATTGTGCCAATTAATGGTCTTAAATC
AGGGTCAGCTAATCCATGCTCTTCTTCTGGCGACCAAGGAGTTTCGCGCTCTTGGTTTTG
TAAAAATGCTTTTAAAAATACTTCAGTTGGCTTCCAAATAATTTCTACTGTATCTCTCAC
ACGATAGTTAAAATCATAATAAGAAATTATTTCGCCAGAAGATTTGTAGAAAAGCATTCC
TGATGCGTATTTTTTAAATCCGGTGAATTTAACATTTGGCATTACTATCGTGCAATCACC
ATCATCCCAGTATTCATGAACTAATCTATCTGGTGATGTTTCTGGAATATTTTCTATAAC
TTTAGTGTATTTTAAATCAGCATAAACTACTACAGCTCTATCAGAGTTGTTTATCCAACA
TCGTGTGTTAGATTTTTTAGACCAGTTAAAGAACGGTTCTGCGTAGTATTTCATTGGTTG
AGGAGTAAAAGTCTCCCAATCAGATTTTTCATCCGCTATAAATGCCATCATATGATAATG
CTTATCAGCTAACCATTCACGAGGAAATTCATATTTAACAGCACCGATTAACTGATATTT
CACCACAGTTTCAGGGTCATTAACAATGTTATCACTTAAAAATTTAAAATTACTCGAAGA
TAGAGAAACTAATTTACCGTCAGTTGACATATTCGCATATCCGGGTTGAATACGTCTTCT
TTCTTCTTCGGTATTACCGAAAACTCTTTTCCACGTTTTCGTATCATGGTTTAAAACATA
TATTCCTTTATCAGCAGAATCAATTATTTTTGATGTTCTAGGATTGGCATTTAATGTTTC
AACTTCACCAATAATAAGAGCAAAAACTTTATCACCAACAGAATCCATTTTATAGCATAC
TGCTTTAGGATTTCCGGTTATAGTCATGGTATCAGGTTCAAAAAGTCTTTCCGAATATGT
TGGAGATAATGGATCAGAATCTATAGGTGCATTACTCGTTTTTATGTATCTAACTTTGTC
TCTGGCGACAACGTAAATGTAATCATCGGTGCAAGTAATAGCTTCTGCAATGCGGTATAC
ATTCGCTGGTAAAGTCGCATAAGTGGCAAAAATTTCTACATCAAATCCTAAATGTAATTG
GTCGCCAAGTTTAGCGAATGTTATATCCTGTGAACTGAATCTGACATCATCAGCTGACCA
TCTAACATCAGTAGATTTGCGGCCATAGAAAATCTTGTCGTATCCTAGAACGTATGTAGT
GTTCGCAGATTGGTAATATACTGTCTTAGATAAAGGATATCCTACACGGTCATTGAAGAG
CTTTACAGCTTTCCAGGTTTGTCCTTTATCATTAGATACTTTAACCACGGGTTGATAACG
CTCAAAAAGATATAGAATCCCTTCTGATTCCATCAAATAAACTCGGTTAATATCCTTACA
TACTTGCTGAATGGAACCTTGTATTTCATGATACTCATTTTCACCAATAATAAAATTACT
GATTGATGAAACATCAACATATGATGGGCTGAATTGGAACGATTCATTCATCAATGCAGC
CATTATAGTGTCATTATTAAAATTGACATAATTAGAATTGTTAAGAGTAAACTTTTCCTG
AATGAATTTATTGGCTAATTGCATTTCAATCATGTTTTGAAATGTGTAAGCATTCGTAGC
AAAAGTTTCAAACTCTTCAGTATAAATCCAATCAGACTGCTCAAAATCCTGCGCAGCTGT
AGCTACTCTAATAATGTACGATGTTAATGGATTAGCATTATCAAAAAAGAAGCTGTTATT
TGCAGTATATCCTAAGTTAATCCATCTGTATTGATTGCTCGGGAGATTTTCTCCCGAGTT
TGTTTTTGTCTCAGCGATTTCTACAAAATAGTAGAAATTAGCACCAACGTCATCCCAGCG
TACTTGCACCTGATTTGCGGATAACTTGGAAATTCTGAGACTAGTGACTGAAGGTGCTTT
TACTGTCATTGTGATATAGGCTCCAAATCGATAGTTAAGTATTGTGGACGTAAATCATTT
TCAAATACAATCAGTGAACCATCGCGCGTGAAAATAACATCGTCTATTGGATCAGAATAT
AGCTCAATAGTCTGAACCTCAAATTTATCGGATGTTAAATTAATTTTAGCGATATTCCAA
TAAATCATGTCAGCTGGATAATTTATTTCACCGATAACATAGTATTTGTCACGTCCATCG
GAACTTGCTAATTTATTAAAATCATTGCCTGTATATGGCTGAATGTTTTCATTTTCTGTA
ACATCGCCAGAAGCAAATGGACCAATAATAACTTTGCCAATTCCTTTAGAATCTCGGTCT
GTTGATACTATATGAACGTCATATAATACATCTTCTTCTAAACCAGTATCAGGATTTACA
ACCTTTCGTCCAGAATTAAATGAAAACGTATTAGATTCCATAGAACGATCTTTTATTTGA
TTATTGTATTTAATACCCGCTTCAGGCGTTTTATAGAAGTTTTGTACTTCACGAACCATT
TGAATAGTCGCTGATGAACCAATGACAGAATGATCTGCATCATCTACATATGTCAACATC
TTAGATTTAGCGAAAGATGAGTTAAAAATTTCTACATCTTCTGTATAATAGCGATCAATT
TTATCAATTATTTGACCTTCAAGCCATTGCTCGGATTCTTGCAGTTTATTTAAAGCATAT
GTGACTTTTAAATTAGTCTTAATAAAAAGATAATTAGGAGAAATAATTGATGGCGTAATA
GGAGCTAAATTATAGTCTTTGAGATAATTTTTAATATCTTCACGCTGTACGGTAGTTAAA
TACAGACCTGATTTAGGTTTGGCAGCAATGAATGCATACCCAGGTTTAGTAGAATCAGTG
AAAGTTTGAACAGCTTGAATAATAGAACCAAATCTTTCTGAGACAAATGTATCATAATCT
GTCGCAGTTACACATCGCTGCTGAGTTTCACGCTTAATAGTACCCAATTCACGAATACGT
TCAATATCTTCTGGATCACCACCACCATCAGCACCAACAAAATCTGGGTCATCGTTTGGA
TTTTCATTAATATTGATGACAGTTATATTTGTTAATGTATCTGCATATGAAAATCCGACT
GCGCCATTCGCATCTGCACCGTTAGTACTGATGTACTCAATAACAATCGTAGAGTTCTGG
GTAGGTTTAAGACCTCCGATATAATTAGCGGTCAAAGCTCCTTCAGAAGCATTAACAGAA
ATTTCACCTTCGCCAAAATAGAATTCAGTGTTTCCATCAATAGTTTCACGCATATAGTAA
ATCGTTGATGTTGAACCAGCATGAACCATTGACTTTCTCGTCCAGTTAATCCATTCCGCT
CCATCAACATATAATTTAACCTGGTTTCTATCAATATTTTTATCATAAATGATAATAGGT
GTTAATTTATCATAAATGATTTCAGTTCTTACTATACGTCCCTGGGCCAATTTCAATCGT
GGAAAATATTGGTTATTTTTATCACGAATAGCAATAACATCTTCGGTAGATACAAAGTTA
TAAGGATTAACAGAAGTATCCTTTGCATATGCTAAAAAGCGAGTTCCGCGAGGAATAGTA
ATGTAATTCCTATTCAATGCGTCGGTGCATGTTAACATAATTTCGGTCTGCGCAGCGGAT
TTTGAAGTAGGTAAATATCCGTTATCTTGTGCAGCTTGAACAACTGAACTTCGTAAGTTA
GCAGTACGCATAAAGCTTTCATACACAGCAGCATTACCAAACTGCTGAATGTATAATGTA
TTATAAGCTAAAAGGTCACACAGAACGTTTAATCTTGAGCCTTCAAAATCATAATCCAAA
AATTCATTTTGGCCATTAAGCCATTCAATGAGGTTTTGTTTTATTTCAGCAAACGTACCC
CCGACGAATATCTCGGGAATAGCATTTGCTGTTCTTGTTAATTGATAATTTACAGGGGTA
TTTGCCATTTTAAATCCTATTTAATGAATACTTTAGATGATGCCTGAGCCACAGTATCAC
CGCATGATATTGGATCAGCCATTTGAACAGCTTTCTTTCCCGTGACATATACCTTAGATG
TTCTAGGTTGTGTCACTCCGCCATGTGTTTCATATGGCTTTTTAATTTCTGTATGTTCTG
TAATTGGATCGCCTGCTACTAGAACAGCAATTCCTCCAGTGAATACTTTACTTTGTGTAG
CATTCACAACTGTTGGAGGCCACGCTTCATGGCCAGCAGTAACACACTTATCATAACTTA
ATCCTGACATTTATGGCCTCTCATACACATAGCTTCTCAATTTATTAGCCCAACGACTCC
AATTTCCAACTATAGTTTTAGTATAATTTTTAACGAGAGTTTTTCTTACTGGAGCAGGAG
GATCCGTCGGTTCAGTAGTATCAGAAGATGACCTAGAATTACTGCCAGAACCTCCAGATT
CACTTTGTTCTTGGTAGTCATATATTAATGTTACTTCGTAAGTGAATGTCTTCTGGAGAT
TTTGAGGAGCTTTCCACAAATACAACTGAGTATCAGAATCAGTAGGAAGTTCTTCCCATG
AAGCAGCAGTTTTAAATTCATCGCCTAAACGATATTTCAACGCGTCATCTCCAAATCCAA
ACACAGATTCATATGTTCCATATAAGCGATTTTCTTCTACTAAAACCCCAGGAGTTTCTT
CGTAACTAGTTATATTTATAGATACTAACGTTTCACCTGTTTCTAATTGAGCGGTAAAGG
TGACGTCGATAGAAGAACCTTCCATGGATTCTCCTAAATCAGCGCTCATTGGAAGTATAT
TAGCCAATGTCAATCCTCGATCCATCAATTGTGTATTGACCAGATGAAATAGAACTCATA
GATGCCATTTTTTCTGTCCAATCACCACCAACGTCCCAATCAACTGTCCCAGCAACTTTC
CAAGAAAGATTTCCATTTACTGTATTAGTTTGATTACCTTCAACTAAAGTGGTAGCATCT
CCTTTAACTGTAATGTCAGCATTACCTTCAACTACAATAGTAACATTACCTTTAACTAGG
ATAGTTCCATTACCTTCAACCGTCTTAGTTTCATCGCCACGTACAAATATTGTATTGCTT
CCATCGATTTGATGTAAACGATTATCCATGTTGTAATAAATTTCTGATCCACCGACGTTA
GTCTTTTTATCACCGGCTACCAAAAAATTACCATCAGCATTTGTTATATCATACAAATTA
TCAACAGTTTTTCTTGTTCTTCTTCCTGACGGTGATACTTCTTCATAAGTTCCAGTTGGA
TGAACTAATCTGTATCGTTCTTGCCCAGGGGTATCATCAAATTCTTGGATATGTCCACTT
TCAGTTTCCATCGTATGCACATAAGGATACTCACCTTTATATGAAGAAACTGGTTCTTTG
AATAAAATTCTCGAGTCATTTGGAATAGGAGGATCAGCAGGGTCAGAAGATTTAGCTACA
GTAGCAGCCATTGCAGATAAAGACCTAGCTGGTGTTTTCACTTCAACACCATATGATTCC
AAATTTCCCGTAAGAATAATCATGGTAACACGGGATGCACGACCCTTTGTTTGTTGATAC
CACAATGAATCACGACCGGCTTTATACGCTTTTTCCCAATCTCCTGCTAACATAGCAGTT
AACATTGTGTTAAATTTAGCTACACCACCAACACCCATCTGAAATGCCATATTTTCTAAC
GCCATTTGACGAGAACGGTTGACAGCTTGCCAGACTGGTCCTACTTTAGAATGTGATTTA
ATGTCACGTTGCATATCAGCCAAATCACGCTCAAATAAAGTCGTCGCCTCTTCCATTGTA
ATAGAACCTGGATTTCCTGTAATTTCACGACCAACTTGTTTTGATAAAACTTTATTAATT
TGAGCCATATCACGAACTGGCTGCTTCATGATAAGATGACCAATACCAATTGTCGGATAT
CCTTCGGTATCCCAATAAACTTTTAATCTTAATCCTTCATCACGGCGAAGCATTTCAGCC
ATTGACATATTTGGATTATCATCGGTTGGAATCTCTGATAGTGGTCTATCATCGGGATTT
ATTGCGGTGTCTAAGTTACTATCTTGAATAACGTTAGAAGAGGAATCATATCCTACTTCT
CCGCCTTGGTTTAATACGTTCGTATCATTTCCTAAACGTCTAGGATATTGCCCAGTTGGA
TCAGAAAATCCTTCAAGTCTATTCGGTTTTTCGCGAACTATTCCACCATACGTGCCAAGG
ACAATTCCATTAGTTTTCCATTTGTCTAAAAAATGACCATAAACTCTAGTTCCTTCTACT
GGCCCTGTAACAGAACCTCCAATTCCTGACATCGCTGCAGAAGTTATAGGTTGAATAACT
GACATCCATGGTAATTTTTCAGTTGGAATACCCATTACATCGCCTTGTGCTCTTTGAGGT
GGATGTAGACCAACCACACGAACACGAACACGACCTAATTTTAATGGGTCCATTCTATCT
TCAACAACACCAACAAACCAATTAAGGTTATTACTTATCATTTCCATAAGATTTCTCCAT
TATACGTATAAGGTCGTTCATAAATGAATTAATGTCTGATTTTGCTATTATTTTTATTTG
ACGAAGTTTTTCATTTTCAAGAACAGCAGCTTCATACGTATCGACCGCAGCAAGTGCTCC
TTCATATTGAGGATATTTTCTAGCTTTATCACCTTTGTCATACCAAACATATGGATTATC
ATCGTATGATATTAAATTATAAAATTTTTCACCGTTCTCATTTACATGATATACTATTTG
GTCTCCACCTACATTTTTGTATTTTTGTATAGATGCTTGATAAGCAGCTTCTTGCGAAGT
AATCCATCCATAATACGGGTCATAATTATCATTACACATCAATAAAACCCAATACAACTG
TGGATTTCCATATATAGTATTTGCTAATTCTTCCGGGCGTGGTGAACCTTTAATATAATA
AGTACGTAAGCGGTATCCCGCAAGAGCACGTTTGAAATAGTCTTTATAGTTTCTAAAAAT
ATCTGTCATAGGAATAGTCGGCGCGTTTTTATTCACCGTTTTGGCCGCATATTCAATCGG
ATCAAAAAATGTAAAGAGCATGGGCCCTCCTGTTTATAAATATATTATCTATTTATAAGG
AGAATCCAATGGCATATTCTGGAAAATGGGTTCCTAAAAATATATCAAAATATAGAGGTG
ACCCTAAAAAAATTACGTATAGATCAAATTGGGAAAAATTCTTTTTTGAATGGTTAGATA
AAAATCCAGAAATTATTGCATGGGGTAGTGAAACAGCAGTAATTCCTTATTTTTGTAATG
CAGAAGGGAAAAAACGTAGATACTTCATGGATATTTGGATGAAAGATTCTTCTGGACAAG
AATTTTTTATTGAAATAAAGCCTAAAAAAGAAACACAACCACCAGTTAAACCAGCACATC
TAACAACTGCAGCGAAGAAAAGATTTATGAATGAAATTTATACATATTCAGTTAATACTG
ACAAATGGAAAGCAGCACAATCTTTAGCTGAAAAGCGTGGAATAAAATTTAGAATTCTAA
CAGAAGATGGATTACGAGCTCTTGGCTTTAAGGGGGCATAATGGCTATTTTTCAAATAAT
TAATGAAAGCACTCCCCAAGTTCCAAAGGTTAAGCAATCATTAAACGAAAAGAAATGGAT
TCAGATAGGTCTTGAATATAAAAAGGCCAAAGCAAAAGGAATGACAGGAAAGCAATTTGC
TGAAGAAAGAGGAATCAAATACTCTACGTTTACTTCGGCAATGTCAAAATATGCTTCAGG
AATTAAAACGGCTGAAAAGATTCAAAAGCTTGAATCAAAACCAATGAATAAGCTCAATAA
GCAAGAAAGACAACTGCTTATGATAAATTCATTCAGGCAAACATTGCGTGATAAAATTCG
TAATGAAGGCGCAGCAATTAATAATAAAACCAGAAAGTGGTTTGCTGAAACTATTAAGCA
AGTAAAAGGACATAAAGTTGTTCGCCCGCAGCCGGGACGAATATATGCTTTTGCTTATGA
TGCTAAACACAAGGAAACTCTTCCTTACTGGGATAAATTTCCTTTGATAATTTACCTTGG
TTTAGGTAAGCATAATTTAATGTACGGATTAAACTTGCACTATATTCCACCTAAAGCTCG
TCAGCAATTCCTAGAAGAGCTTTTAAAGCAATATGCAAATACACCTACTATTACTAATAA
AACGAAATTAAAAATTGATTGGAGTCAAGTGAAAGGATTTAGGGGTGCAGACCAAATGAT
TAAGGCATATATACCTGGTAATATTATGGGTAGCCTTGTTGAAATCGCCCCGAAAGACTG
GGCGAACGTTGTGTTGATGCCACTTCAGCAGTTCGTTTCAAAAGGAAAACGTTTCTCTGC
AAACAAAGTCTGGTCAAATATCTAATTCTATTATCTTCCATTCTTTTCTGTTGTTTGTTC
TAAATGGAATTGAATGGAAGGGACTTAGACCCATTATACCACCAACAGTTATAAAGCATT
ATGAGGAATATATGTCGCAAGCTCTGCAACAAATTTTTAACCAAGCAAATACAACTAACT
TTGTAGTATCAATACCACATAGTAATACTACATCTGCTTTTACTTTAAATGCTCAGTCAG
TTCCTATTCCAGGAATTAGAATACCTGTTACTGATACTGTGACTGGGCCGTTTGGACTGG
GCCGAGCACAACGTCCAGGTGTTACATTTGAGTATGATCCACTCATTGTGAGATTTATAG
TTGATGAAGAACTTAAGTCGTGGATAGGAATGTATGAATGGATGCTAGGAACTAGCAACT
ATCTTACAGGTGAAAATACTGCCCAAAAAACAGGTCCTGAATACATTACGCTTTACATCT
TAGATAATAGCAAAACTGAAATCGTGATGTCAATAAATTTTTATAAGCCTTGGGTTTCTG
ACCTATCTGAAGTAGAATTTAGCTACACAGAAGATTCAGACCCGGCTTTAGTATGTACTG
CAACGATTCCTTACACCTATTTTCAAGTAGAAAAAGATGGTAAAATTATAGCAGAAGTTT
AATGCTTCAGTTTTGTGTGTTATAATCTTAATTAAATTTGAGGAGAAACACATGAAACTA
ATCTTTTTAAGCGGTGTAAAGCGTAGTGGAAAAGATACTACTGCAGATTTTATCATGAAT
AATTATTCTGCAGTTAAATATCAACTTGCTGGTCCTATTAAAGATGCATTAGCTTATGCA
TGGGGAGTATTTGCAGCAAACACTGACTATCCTTGCTTAACTCGTAAAGAGTTTGAAGGA
ATTGACTATGATCGTGAGACTAATTTAAATCTGACTAAATTAGAAGTAATCACAATTATG
GAACAAGCATTTTGCTATCTTAATGGTAAAAGCCCAATTAAGGGTGTGTTTGTTTTTGAT
GACGAAGGACAAGAATCAGTTAATTCCGTAGCATTTAACAAGATTATTGACGTTATAAAT
AATATTGAAGATCAATGGTCAGTCCGTCGTCTGATGCAAGCCCTAGGTACGGATTTGATT
GTTAATAACTTCGACCGCATGTACTGGGTAAAATTATTTGCTTTAGATTATCTTGATAAA
TTTAACTCAGGTTATGATTATTATATCGTTCCTGATACTCGTCAAGATCATGAAATGGAT
GCGGCTAGGGCGATGGGTGCTACAGTAATTCATGTAGTTCGTCCTGGTCAAAAATCCAAT
GATACACATATTACAGAAGCTGGATTGCCAATTCGTGATGGTGATTTAGTAATTACAAAC
GATGGTTCTCTTGAAGAACTTTTTTCTAAAATTAAAAATACACTAAAGGTACTATAATGT
CTGAACAAACTATTGAACAAAAACTGTCTGCTGAAATCGTAACTCTGAAATCTCGTATTC
TTGATACGCAGGACCAAGCGGCTCGTCTGATGGAAGAATCTAAAATTCTGCAAGGAACTT
TGGCTGAAATTGCTCGCGCAGTAGGTATCACTGGCGATACTATTAAAGTTGAAGAAATCG
TTGAAGCTGTCAAGAATCTTACTGCTGAATCTACTGATGAAGCAAAAGATAAAGAATAAT
GGAATTTAAAGACTTTTCAACGGGTCTTTATGTAGCAGCTAAGTTTTCAGAATTAACACT
TGATGCGCTGGAAGAACTCCAGCGCTCTTTACGTGTTCCTAATCCAGTTCCTAGAGAAAA
AATTCATTCGACTATATGTTATTCGAGAGTAAATGTTCCGTATGTTCCATCAAGCGGAAG
TTTTGAAGTAGCTTCTTCTGGACATTTAGAAGTATGGAAAACACAAGATGGATCTACTCT
TGTACTTGTACTAGATTCTGAATATCTGCGCTGTCGACACATGTATGCGCGGGCGCTAGG
TGCTACACATGATTTTGACGATTACACACCGCATATAACATTGTCTTATAATGTTGGGCC
CTTATCATTTAGCGGTGATGTACAAATTCCGGTCGTATTAGATCGTGAATACAAAGAACC
TCTTAAGCTTGATTGGGCAGACGATTTAAAATAATTTCACAAAGTTGTTTACACACTGAT
GAGGTAGTGATACTATTACCTCATCAAAATTAATTAGGAAAAATAAAATGAAAACTTATA
AAGAATTTGCTACAGAAACTATTATCACTGAATCTTCTCATGGTATGGAAGTAAAGCTTG
GAATGGCTTTAGCTGAAGCTGAGCGTCTTTTCTCTCGTATTAAAGAACTTGCTGCTGCAG
TCGATCCTTCGTCTTTTAAAGGAGACCAAACTAAAGTTAAAGCACTTTTAGCATTATGCT
CTGACGCAGGAGAAATCGCTAAAAATGGTTCTAAGATGAAGAAACGATTAGAAGATTTAA
AATAATTTCACAAAGTTGTTTACACGGGGTTTTAGTAGTGATACTATTACCCTATCAACT
ACTGAGGAGAATAAAATGAAACGTTGTGAATTAATTCGAAATGTTGCTATTGCAATTTCT
GCTTCCGCTTTTAGTTTTTCAATGTTTGTTGGATTTATATGCGGATTATTAACTACAGCA
GAAAATGTGTTTTCGCTTGTAGTAGCATTTTTAATTGGTTTAATCGCTATCGTTATGGAT
AAAATTTCTAAAGGTGAATAAATGAGTATTAACGAATATTATGTTTATGCCGGGGATTAT
GCAAATCCTTCTCATTTTGAAGGTAATTTAATACCTGATAAAGTTTTTAATACTCCTTTT
GAAGCGTGGAGTTGGATTGAAAGTAAAAATGGATTTTCTTACCGTTATGTTGAAGTAACT
GATTGGAGAGGAACTAAATACCCTAAAGACCATTATTACGTAAATCCATCTAAGGTAAAT
TTTCTTTTATTCGCAGGTGATAATTATTATCCTTGCGGTGGATATGATGATTTAATTGCG
TATGCTGAAACAGAAGATAAGCTGCGTGATATCATTAAAGAAAATGAAAATAAACCAGAT
TATGATTCCAATCGTTTCGACTGGTGGCAAATCGTAAATGCCCATACTCATGCTATCGTT
GATAGAGGCTGACAATGATTCTTTATGCTAAAGTACTACCTTATAGGAATGATATTGCTT
AAATTAATATTGAGGTAGCTGAGACTATGAAATTAATTAAATCGGGTGGAACCAGATGGG
TACTACTAACACGTAAGTATGCTATTAAGTTCCCTATCCCCAAGACATGGAAACACTTCG
TGCAGGGTATGCTCAGCAATCTTACAGAAGGTCAGTGGAAGGGGTATGATAACAAGCACC
TATGCCCGATAGCATACTCAAACAGATTTGGTCTCATGGTTGTGATGCACAAAGCAGAGC
CAGTTGAGGATAAGGAGCTTTTTAAATCTGATTTGCAAAAGCTCTATAATGTTGATGGTG
ATGAATGTAGAACGCTTGACCGTGATTTCTTTGAGTATGATGCCTTTCCAAAGAACTTTG
GCTACTACAAAGGTAGATTGGTCAAGATAGACTACGGAGTGTGATAATGATTCTTTATGC
AAAAGTATCATCCATTGAAAATGGATATAAATATGATCAAGACGCGGCTAAAGCCTTGAT
TGATGATTATGGCGTTTTAACATGTTTTGAAGTTGAAAAGGTTTACATTGACCGTTCATC
TTCACAAGTTAAATTAGTGAAGGAAGACCGTAAATTTAATACAGTAAATTTTGATTTCTT
TATTGAAACGGAAAAAGGTCCTCTTGAATATGATATTTTCAAGAATCCTTTGGGTCTTGA
ATGTATTGAATATACTTACATTAATATGGTGAGCAAATGAATATTTGTTTAGGTACTACA
ATTCCACAGGGTTACGTAATTGATGTCACTACCTGGGAAAATGATGGTGACAACTATAAA
ACTAAAACACTGTTTGGCGTAGAAGAGCATGAAATCTTACAATTTCAATATCTTTTAAAG
AAGTTTAAAAGTAGACATTCTAGTACTAAAGCTGAACGTTATTGTGGTAATGGCTTGTTC
AGCGAGCAATCGCTTTTTATTTATGAATACTTGGTTGAAGGACTGTTCTCAGAACAACTT
TATCCAGAATTCATTAAAAAGGTCTTTGATATAGAAGTTGATCTTGGTAATAAATCTGAA
GAAGATGAAGAACGTGTATTTGACTTATTTTTTGTGAATGGTAATAAAGTGCTTGAAGGA
CTTATTAATATTTTAGGTAATGCTTCTGAGTATTATGATTATGATTTTTTGCGTGTAGTT
GAGAGTGTTAAATACTCTTATATAGAAGAAGAAATTGTTTTGCCGACCGTTAAAATGTTT
AATTTGCTTTAAGAATTATTTGTTATTATTAACTCATACCGCACTGATTAATACCCTCTA
TCATCAAGGGTTCTTGTTTGAGAGCCTTTGTTAATAATTGGGAATTAGCCAAGTTGGTAA
GGCATAGCACTTTGACTGCTAGATGCAAAGGTTCGAGTCCTTTATTCCCAGCGCGAGAAT
GGTCAAATTGGTAAAGGCACAGCACTTAAAATGCTGCGGAATGATTTCCTTGTGGGTTCG
AGTCCCACTTCTCGCACCAAATTTGCGGATATCGTATAATGGTATTACCTCAGACTTCCA
ATCTGATGATGTGAGTTCGATTCTCATTATCCGCTCCAATTTAATTTACTCCGTGTAGCT
CAGTTTGGTAGAGCGCCTGATTTGGGATCAGGAGGTCCAAGGTTCAAATCCTTGTATGGA
GACTGGAGGCGTGGCAGAGTGGTTTAATGCACCGGTCTTGAAAACCGGCAGTCGCTCCGG
CGACTCATAGGTTCAAATCCTATCGCCTCCGCCAGTTTTGCTGATTTAGCTCAGTGTATT
GAATAAGTAGAGAAATTTGTGATGAAAGAAGATTATGCAAAATATATTTCTCAGCGGAAT
TCTGCTATACGTAGAAACATTGAATGGAATTTAACTTTTGATGAATGGCTGCATTGGTGG
AAATCAACGGGAAATTATGATAAGCGTGGAAGAAAGGGTCATGAATACTGCATGTGCCGT
TTCGGCGATAAAGGACCATATTCATTAAATAACATATATTGCGCAACTAATAATCAAAAT
ACAAAAGATGCGAGACTAAATGCTAGATTTCCTCCAAAATCTAAAAATTTTAATTTTAAT
GGTCGAAAACACTCAGCACAGTCCTTAGAAAAAATTTCTAAAAATAATGCAAGTACCTTA
AGTCAAGATGATATAACTAGACGATTAAAAATATTAGAAAATTTTAATATGGATGAACGA
GGTTTTATTAAAAATTATGCAAACGCTATAAATGTTAGCCATACTCAAGCTAGAAAGTTT
TTAAATAAATATTACATAAAATAAGAAAATTTGGCCCTGTAGCTGGAAGGTTCAAGCAAG
CGACTCATAATCGCCAGATGGTGGTTCAATTCCACCCAGGGCCACCAAATTAATTTGGGG
AGTTATCCCGTAGAGGTAGCGGTGTGGACTGTAAATCCATTGTCATTGCGACTCGGGTGG
TTCGACTCCATCACTCCCCACCAATTTGGATGTGTAGCTCAATGGCAGAGCGATCGCCTG
TTAAGCGATTGGTTATAGGTTCGAATCCTATCACGTCCGCCAAATCCGAGGCATAGCTCA
GAAGGAAGAGCAAGGACCTTCTAAGTCCTAGGTCGTAGGTTCGATCCCTACTGCCTCGAC
CAATTAAGTGAGGAAAATATGAAAGGTAATGTTTATTTAGTAGTTCATGATTTAACATTC
TATTTTAATCATAATGACACTGTTATTTCTGAACGTGTAATTAATTTGCTTTATCAGCAC
GCAGATTATGTTTATGTCGAAAACGAATACGGTCATTGGCAATTTCTTAAAAATCGTTCA
TTTGGCTTAGATGGTTATGAATACTTTGAACATAAAGACCTTTTAGATAAAATTCCGTTA
TCTACGCAATATCAAAATCACAAGTCTTTATATAAATGCCGTCCAATTCGAAATGCTGAA
TCTGCGTATGAAGCAATTGATTTATGGCGTAAACGCCGTGAGCAGATTGATGCTTTAAAA
GAATATTAAGAAACCGGGTCGCTACCGGTAAGTCGTCGGACTGATGTTCCCTGAGTAAGG
AATTGCGTTAATAATCTTTGCGTTTATTGATGCCCTCTTACATCACAGCAGAAACGGCGC
ACCGAATTATCGATTCGAGGAAATATCTTTGCCGTAAGCCGAGTAGCGTTTTTGACGGAA
CGTTCGGATATGGTCGAGATATGGCCTTTTAAAAATATTAAGTAGCGTCAACTGCTTAAT
AACCGGGTTCGAATCCCGGCGTTTCGTACAAACACTTGCCTTAGCAGGTGGAACCCCGAC
AAGGTTGCCGCAAGGCTTAGCCCCGACCGAAAGGTTGGGGCTTTTTAGTATAAATACAGT
AGTATATTAAATCCACGAATTAAAACAGGAAATAAGATGAAATCATATGCTCAATTTTTA
AATGAAGCGGTGTTAAATGAAGCATCTAGCACCGAAATTCAAGCTGTCGCAAAAGCAGCC
ATTGCAGCGGGTAAATATTCCTATAAAGATGCTTCTGACGAATCACGATTCCAATTTGCT
CGCGACATGAAAGCAGAAGGATTTACGGGAAATGCAGTTAGTATGGCCTGGAAAAGTCTA
GTTGCTACTGGCGCTGCTTTTGCAAAGGCTTCTGGTAAACCTGCTCCTAAAGCAGATCCT
AAAGTCACACAAGAGAAAAATATCGTTAAAGGAATTATCTCCAAATATGAAGCTATCCTT
AAAGAGCTTTTAGTAATCAAAACTGAAGGCCAAAAGCTAGCTCGTGCTTATAGTTTCAAA
GATAACCCACATGCTCACTCTCTTGAGTATGTTGAAGACATCCAAAAAATTATTAAAGAC
CGCATTTGGTCTGCTAAACAAATCAAATAACATTCTTAGCCCTAGCCGAAAGGTTGGGGC
TTTTTGGTATCTAGGCCTTTCCAGGCCTCTCTAGACATCATTTAGTTTCTACCCTTTATA
ATATATTATCCTATCCTTTGATTTCCCCTCCCTGCCCTAGAATTCCCCAAAACTTTTTAA
AAATTTTTTCACAAAACTGTTTACATCCCTGTTCTTCCATGGTACTATACAACTATCAAC
TACTGATACAGAAAACAACTTGGAGAACGAAATGGATAATTACGGCGAACTGTTCAACTT
CTTTATGAAATGTGTTTCAGAAGATTTCGGTCGTACAGTGAATGATATTAAAGTTATCGG
TCCTGATCATCCGATGTTTGAAACTTATGCGGTAATGGGCAATGAAGACGGTCAGTGGTA
TACTGTAAAGGTTGTGATTAACATGTTCACTGCTGAAAGTTATGTTAAACTGTCTTCTAA
AGTTTACCATGATAACGACGAAATCGCAGAAGAATATTTCAATAATATGAAATAAGTTTA
CATAGGCTCATGATTGAGATATTATGAGCCTATAACTTTGAGGAAAACATTATGAAACGT
AACTTTAGTAAAGCTTTTTGTAATAGAAATAATTATCCTGAAGAGCTTTTTGAATCTAAG
GACTTTAAAGAGTTTAACGAACGTTCTCAAAACATGCCTGTACAATGGGTGTTGGATTAT
GCTAAACGTATCTTTGAAGGTGTACGTTGGTTCAGTCCTGATGATGAAGCCCCATATTTT
CAAAGGGCATGGTCTATAGTCAATCAAGCTTATATTGATGCTTTAGAAAATATTAATATT
ATAGATGATGATGCTTTGTTAATCATCGATGAGTCTATTAATAGTAGTATCGATAAATTT
ATTGATGAATTGGACGAAGAATAAGTTTACAAACTCTTGGGAGTAGAGTATAATACTCCC
ACGGATTATAAAACCCTTAATTAACTAAAGGAAATGCTATGAACACACTGAAGAAAATTG
TTGAGTTTATTCGCACTAAACTTGGTTCTGCTATGGCTAAAAATCTATCTGTTGAAGAAC
AGTATACTGCTGCAGCAGCAAAACTACTTGATAAGATTAAAGATCTAAAAACTGCTTCTG
TTAAATCTATTAATGAAGAAAAGCGTATTCGTGAACTTATTGTCGAAAAGAATAAGCAGG
CCGAATCAAAAGAGCGTGAAATTCGCAAGCTTCTTTCGGAAGGTCAAGATGTAACAATGC
ATGCTAAACTTGGTTTACTATATCGTCGAACAGCTGAGCAGTTGACTACTAAAGCTGATG
GTTATGCTGAAATGCGAATTGAAATCGCCAAGAAAGTAGTTGAGTTAGATGATGCTCGCC
AAGAACTTGCAGTTAAATTGGAATATATCCGTGAAACTCGCGCCGCGAATGCCCTCGGAA
TTAGTACTGCTGATGATGTAGTTGAAATTGCAGCGCTGACTAAGGTTGATATTGAAGATA
CTCTTGCCCGAGTTGAAACCTTTAATGGAAATATTTCTGGGGTTGAAACTACCTCTGCCG
ATGTTCAGGAATATATTAATTCTCTGAAATAATGATAAGGGGCTTCGGCCCCTATTACTT
GGAGTAAATAGGAATGAAAATGCAAAGTGATTTCAATTCAATGTTTGAAGAGTTCCAAAG
ACAGGTTGATGTTCCAGACCAATTACTAAATGCTCTTAAACGCATGGCAGAAGGACGTAA
TTACTATTGGGGGTCTTCATATGAAACTGATGAAAGCCTTTCCGGAAGATTTTCTAGAGG
TAAAAAGTCTTTAATACGTCCTGGAATACTCATTAACAGTATTGAATCAATTCATTCATT
GACGTGTGATTTTGATGTTGAATTTACTGATTTCATTTCTCCTGAATGGACGGTTTGTTA
TTTAAACGACGATTTTGATTATCTCGGTGTTTATAGTTTAAGTGACGCATGGTTTAAACG
TAATTTACAAAAGTCAAATTTATTTTATATTGATACTACTGTAAAATTTCAGGGCAAGAA
ATATTTCTTTACTCTTATAGTTGATTCTGAAACGAAGCATGAAAATAAACGTATTCTTAG
TAAAAAGAATATCTTAACTATTGTTGATGATCTTTTTGATAAATTTGTAGAAAATCCCAA
TTTTGAAAGTGATTTATTACTAGAAAAATTTGTTAAGGAATGTAGAGAATATGTCAAAAC
CATCACTATACCTTCCAAGTAAACCTGTGAAGTATGAACCAAAGCGTCAGATAATTTCTA
CTGATGTGTTAATAGGTCCTGTTATACTCATATCATTTGTAATTCTATTGATTATTGGAG
GTGTTTTAGATGTTATGACTGATATTGATTCTGGCGAAATACTTGTGTTAATGCTAATTC
TTCCATTAATAGTTCCACTTTTATTAGTACCCGTAAATTGGGTAGGATACTGGTATCAAG
GAAGACATTATCGTAAACGTGTACGTGATTGGAAAGCTCAGTGTAAAAAGATTAAAAAGG
AACACCAGCTTAAGCTAGATATGTATGAATTTGATGAAATTATGAAATTTGTTAAGGAAT
CACGATGCAAAAGCCAAAACTAAATAAAGTCAAATATTCGTTTCCTGAGGCACTATTAAT
TCTTGCTGTATCAGTATTTACAGCTCTTGCGGGTAGTCTTATTGGATTGTTAATTGACTG
TTTTATTTTAAATATCGACGGCACAGTAATTATAACAGAGGTTTGGAGTGAACTTCGTTT
TACTATAGCGATTTCATTATTTTCATTTTTTGGTACCATGTTATATTTTCATTATGATAA
TTTTAAAATAAATTGGCAAAGAAAAAAGGATTACAAAATACAATTAAAAGAATATAATAG
CTACATGTCTTATATTGAAAATGAGTCAATGAAAGAGTTTGTGAGTGATTGTAGGAAAAT
TAAATGATTTTAAAAACTCGCTGGTATGATTTAGATGATGGGGATGATGGCATTTCAGTT
GATAGAGTTGACTGGAATGGTTGTTCCGAAGATATGAAAAATTTGTTGATTAGGGAGTTT
AGAATGGGATATCAAGCAGCTAAGCCATTTACTGTAACAGATGATAAGTTCGTATGTATT
CAAAACGGTCGCGCTAAGTTAACGAATGCTGATTGGTTCACAGATAAGAAGGTTCTTCTG
TGGTATATCATTAGTCTTCCTATATCATCGTTCGTATTTTACTTTTTTATAAAAAATCCA
ATGGACAGAATAGGAGATTGGGTTCTTTTAACTATACTTGTTAATATTTTTACAGCAGCA
GTATTATCAGGAATATGGCGTATGTTCATTGAAATGCCATGGCGTTTACGCAGACAACAA
AAGATTTTTGATGAAAAGAAATATACTCAAAATTTAAATAACTTTATCACTGAATGCAGG
AAATTAAAATGAAAACACTATCAGCTGGTATTATCTTTATGACAGAAGATAAAGATTTAT
TTATGGGTCGGGTTACTGGTTCTCGTAAGCCTGGAATGATGGCACATCGCTGGGATATTC
CAAAGGGCCGCGTAGAAAGTTCTGATTTGAATGCACTGGAAGCTGCAAAAAGAGAATGCT
CAGAAGAGACTGGTTTTATCGATTATAATCCAGACCTTCTAGAAGACTTAGGTGTATTTA
AATATTCTAGTAATAAAGACTTACAGTTATTTTATTATACGATTCCAGTAGAGCATGAGA
TGTTTAGAAATTGCCATTGCGAGTCTTATTTTGAAAATAAAGATGGCGTTATGATTCCAG
AGATGGATGCTTTTGCTCTTATTCCTCGTACTCAGTGGCAATATGTGATGGGTCCTTCAC
TTTACCGAATAATGAACAGCCTCTTTTAATTTATAAATACCTTCTATAAATACTTAGGAG
GTATTATGAATATATTTGAAATGTTACGTATAGATGAAGGTCTTAGACTCAAAATCTATA
AAGACACAGAAGGCTATTACACTATTGGCATCGGTCATTTGCTTACTAAAAGTCCATCAT
TAAGTGTTGCTAAATCTGAATTAGATAAAGCTATTGGGCGTAATTGCAATGGTGTAATTA
CAAAAGATGAGGCTGAAAAACTCTTTAATCAGGATGTTGATGCTGCTGTTCGTGGAATTT
TGAGAAATGCTAAATTAAAACCAGTTTATGATTCTCTTGATGCTGTTCGTCGTTGTGCAT
TAATTAATATGGTTTTCCAGATGGGAGAAACCGGTGTGGCAGGATTTACTAATTCTTTAC
GCATGCTACAGCAAAAACGTTGGGATGAAGCAGCAGTTAACTTAGCTAAAAGTAGATGGT
ATAATCAAACACCTAATCGCGCAAAACGAGTCATTGCAACGTTTAGAACTGGCACTTGGA
ACGCGTATAAAAATCTATAAAGTTGTTTACTTTCTCCTAGAATTGTGATAGTATATTCAC
AGTTACTTGGAGGGATAAAATGACTCGTATTAATTTGACTTTAGTATCTGAACTTGCTGA
TCAACATTTAATCGCAGAATACCGTGAATTGCCGCGTGTTTTTGGTATAGTTCGTAAGCA
TGTGGCAAACGGTAAGCGCGTTAAAGATTTTAAAATATCTTCCGAGTTTATTTTAGGTTC
TGGTCATGCTACGTTCTTTTACGATAAGTTAGAATTTTTGCGAAAGCGTCAATTAGACAT
TATAACGGAATGCTTAAAACGCGGGTTCAGTATAAAAGATACTGAAGTTCCTGACATCAG
CGATATTCCAGTAGAATGGAAAAATGATTATAATCCATGCAAATCAGCTATTAAATTGAG
TCAACAACGACTTGATGAAAAAATTTTAATGAAACCACATTGGTATAAGTATTACGGTAA
AAATATTTACATTTAAAGGAAACCCATGAAAACATATCAAGAATTTATCACTGAAGCAGC
TATTAATTCTCAAATTATTGCTGAATCTTTTACTGATCTTTTGAAATTTAAAAAAAGGTC
AAAAAATCACTGCTGTATTGGATGATGGTACAGAAGTTGAGATGGATGTACAAGGATATA
ATTATGCAGTAGATGGAAAACTGTATAATAAATCTCATGCTAAATTTGATTCATTTGACG
ACTTTGTTAATACAGTTGAAGATGAAAAAACTCGTCGATCCATTGCAACTGGTGATGCTA
AGGTTCTTATGGCACATGGTCATGAACGCATTCGCGCTAAACAGAATAAAATGGGTGAAG
ATAATTTTGCATTAGTTGGTTATCAATCTGGTAAACAAACCTATGGTTATCAGCGTACTG
CTACCATGTATAATAAAAATGGTAAAATTGCCTTTGTGAATAGTAAGGGTTCTATTCAGT
ACGTTAAATCGTTCAAATAATATGGGAACAGCCTGGACCTCATGATTCTATGAGGTATTC
CCGCCAACCTGTGATAAGGTCGAGCCAAAGCGCGGTAATGGGTAATGCAGGAATGGACAA
CTCATGTGCCATGGAATGGCCCAAAAATTAAGAGAGAAAATATGAGAACATTTTTAACTG
GCCCGTATCTATCCCTGATGAATGCTTTTACACACCATTCTGATGCTAGAGTAGAAGAAA
TTTGTAAAAACGAATATATCCCGCCATTTGAAGAATTACTTAAACAGTACTGTACACTTC
GTATAGATGGTGGACGTCAATCCGGTAAATCAATTGCTGTGACTAACTTTGCTGCTAATT
GGCTGTATGATGGCGGAACAGTTATTGTTCTATCTAATACTTCAGCTTACGCTAAAATTT
CTGCAAATAACATTAAAAAGGAATTTTCGCGTTATTCTAATGATGATATACGTTTTCGTT
TATTTACTGATTCTGTGCGCAGTTTTATTGGTAATAGAGGTAGCAAGTTCAGAGGTTTAT
CGCTTTCACGAATTTTGTATATAATTGATGAGCCTGTTAAATCTCCTGATATGGATAAAA
TTTACGATATTCATATTGAAACTGTGCAACATTGCTGCAATAGTAAATGTTGCATTGGTG
GTATTACTTGTCCACAGTTTTTCGTAATCGGAATGCAATGATGACAGACACTCAGCTTTT
CGAATATCTTTATTTTTCGCCAAAAACTATTAAAAATAAATTGGTGAATCATTTTGAAAT
TTTAGCAAATAATAACATTTTGAGCGAATTTTATCCCAAGCAATACAAATTACAAAAAGG
CGTATTCAAAGGATGCAGAGTTTTGTGTACTGCTCCTAATGCACGGCTAATGAATAAAAT
TCCATATTTTACCATGGAATTTATTGATGGGCCTTTTAAAGGACTAATCACCCAAAGTTT
AATGGCATATGATTCTGAGCCATTTTTAATTAAAGAACAATCTTGGATAAATTTATTTTC
TAATTGAGGTTTATATGAAAGCATATCAAATTCTTGAAGGCACACATAAAGGTACTATTT
ATTTTGAAGATGGTATTCAAGCACGAATTATTGTCTCTAAAACCTTTAAAGAGGACTCTT
TTGTAGACCCAGAAATTTTCTATGGTTTGCATGCCCGTGAAATTGAAATTGAGCAACAGC
CTACAGTTAAAATTGAAGGTGGTCAACACCTGAACGTTAACGTTCTGCGTCGCGAAACTC
TGGAAGATGCAGTTAAGCATCCGGAAAAATATCCGCAGCTGACCATCCGTGTATCCGGTT
ATGCAGTTCGCTTTAACTCTCTGACTCCGGAACAGCAGCGCGACGTTATTGCTCGTACCT
TTACTGAGAGTTTGTAATGGCAAAGATAATTATTGAAGGTTCTAAAGATGTGCTAAATGC
TTTCGCCGAGTGGTTTAGTAATTCAGGCGAACAGCAATTTAATGAAGCCTGGAATATGGG
TGATATTGATGGAATTTATCCTACGACAGAAATTTCTGTTCAAGGATATGGCATTCATGA
ACCTATTCGTTTAGTTGAATATGATTTATGTACTGGTGAGGAAGTCAAATATGATTGAAG
ACATTAAAGGTTATAAGCCGCATACTGAAGAGAAAATCGGTAAAGTGAATGCTATTAAAG
ACGCTGAAGTTCGTTTAGGACTTATCTTTGACGCTTTATACGATGAATTCTGGGAAGCAC
TAGATAATTGTGAAGATGATGAACTCGCGAAGAATTATGCTGAAAGCCTCGATCAGTTAA
CTATTGCTAAAACGAAACTCAAAGAAGCCAGTATGTGGGCTTGTCGTGCAGTGTTCCAAC
CAGAGGAAAAATACTAATGGCTCAATTAAGTGCAGGGTTTGGTTATGAGTATTATACTGC
ACCTCGTCGTGTATCTGCCGCTCCTAAGAAAATTCAAAGTCTTGATGACTTCCAGGAAGT
AGTCCGTAAAGCTTTTCAGGACTATGCACGTTATCTTAAAGAAGATTCACAGGACTGTCT
CGAAGAAGATGAAATTGCTTACTATGAGCAGCGTCTTGAACAGCTCAAAAACTTACATGA
GGTTCGTGCAGAAGTTTCAAAGTCTATGAATAAATTGATTAGATTTAAAGAATAACTGTT
TACTTTTCCTCTTGACTGTGGTATAATTTTTCTATCAGTTAAGAGGAGAATAACATGACT
ATCAATACAGAAGTTTTTATCCGTCGAAATAAGCTTCGTCGTCACTTTGAGTCGGAGTTT
CGTCAAATTAACAATGAGATTCGTGAGGCATCAAAAGCAGCAGGAGTCTCATCGTTTCAT
CTAAAATATTCTCAGCATCTTCTTGATCGTGCAATTCAACGGGAGATTGATGAGACATAT
GTTTTTGAATTATTCCATAAAATAAAAGACCATGTTTTAGAAGTTAATGAATTCCTGAGT
ATGCCTCCGCGTCCTGACATTGACGAGGATTTTATTGATGGGGTTGAATATCGTCCTGGA
CGTTTAGAAATCACAGATGGAAATCTTTGGCTTGGATTTACGGTTTGTAAGCCTAATGAG
AAGTTCAAAAACCCGTCACTTCAATGCAGGATGGCAATTATCAACAGTCGTCGTTTACCA
GGAAAGGCTTCTAAAGCAGTAATTAAAACTCAATGAGGTAAGCATGAGAAAAGCACTACT
CGCTGGTCTATTGGCCATTTCAATGATGGCACATAGCTCCGAGCATACTTTCAGTAATGT
CCAACTCGATAACATGCGTTACGCGTATCAATTCGGGGAACAATTTTCTAAGGATGGAAA
ATATAAAACACACAAAAATATCCATAAGAGCGGATTAGGTCATATAATGGCTGCTATTTT
ATGGCAAGAAAGCTCTGGCGGAGTTAATTTAAAATCTAAACCAAAGCATCACGCCTACGG
AATGTTCCAAAATTATTTGCCTACTATGCGAGCAAGAGTTAAGGAACTTGGTTATAATAT
GACCGATGCTGAAATAAAAAGAATGTTGAATAAACGGTCCAATTCAGCTTCCTGGGCGTA
CATTGAACTTTCTTATTGGTTAAATATACATAAGGGCGATATAAGAAAAGCAATATCCTC
TTATAATTCGGGATGGAATGTTAAAGCAGGTTCTAAATATGCTTCTGAAGTCCTAGAAAA
GGCTAATTACCTTAAAAATAATAAACTTTTGGAAATAGTAAATGACTAAAATTTTGGTTT
TATGTATAGGATTAATTTCATTTTCTGCGTTAGCAGATACATCATATACTGAAATTAGAG
AATATGTAAACCGCACTGCGGCAGATTATTGTGGGAAAAATAAAGCATGTCAAGCTGAAT
TTGCACAGAAATTAATATATGCATATAAAGACGGAGAAAGAGATAAATCAAGTAGATACA
AAAACGATACATTATTAAAACGATATGCTAAAAAGTGGAATACCTTAGAATGTTCAGTTG
CGGAGGAGAAAGATAAAGCCGCTTGTCATTCAATGGTTGACCGTTTGGTAGATTCTTATA
ATCGAGGATTGAGTACTAGATGATTGTAAAATATATCAAGGGCGATATTGTCGCCCTTTT
CGCTGAAGGTAAAAATATTGCGCATGGATGCAATTGTTTTCATACTATGGGTTCAGGCGT
AGCGGGTCAATTAACTAAAGCTTTCCCTAAAATTTTGGAAACTGATAAATTACAGACTGA
ATGGGGTGATGTAACTAAACTCGGTTCTTACTCAGTCTATGAAAAATACTTTAGGACTCA
TAAAGCTTACTGCTTCAATCTTTATACTCAATTTCAACCAGGGCCAAATTTTGAGTATTC
CGCTTTAATGAATTGTATGTTAGAATTAAATGAGTTTGGTGAAAATAAACTGATTAAACC
TACAATCTATATGCCTAGGATTGGTGCAGGCATAGGTAAAGGGAACTGGGATATTATTGA
GGGGATTTTAGATACATATTCCTCTAAATTAGAAATTGTGATTGTTGATTGGGAACCGTT
ATTATGAATATACATTATCCACATCCGTATGACCCAAAGAATAAGGCAGTAATTATTCGT
CAATGGGAACGCATTTGCCGTACTAAATGTCCAATTAATAGTCCACATGATGTAGATAAA
GACTACATTGGAACATTCGTTGAATATACCTTTATTGATAAGAAAGGTCGTAAACAGCAT
GTAGAAGAATACTGCTTAAAGGTGACATGGTTATGAGCCAAACTAGTATTCTTAAAAATG
CCCACTGTGAAAAGTGTAAATGGCCGGTTGTTTTTGCTTTATGCAACGATGAAATGGCTT
GTGATTTCGATTATTGGTGCTATTGTTCTAATAAAGGATGCATCAATCATAAAGGTGAAG
GATTTTATTCGGGATTTTATCCTTATCCAGATTTCGTTAAAGAAGGTAAACCGAAATGAA
TAGTTCTGAGTTACAATATGAAGTGTTACGTGAACTTGATAATTTAATTGAACTCGCTGT
TAATAAAGGTTTTGCCATTGGAATTGGTCAAAAAGATACTGATCATTTAACTGTGGAAAT
ATTTAAGCAAAAGCGAATTATTTTAAAACTTTTGGAAATTAATATATGAGTTTAAGCAAA
GAACAAAAAGACAAACTCTTTTCTCTTATCCACGAAGTTATGGATAAAAACAGTGAATTG
GAAAAAGTTTGTGATGAATGCGGTCCTTTTAATGCAAATGAATACGAAGAACTTTCTAAA
GAATTCGATAATAAAGAACAAGAACTCATTGATTATATAAATTCTTTATGATTACTCGCG
AACAAAAGAACGAAATATTATTTTTAGTTGGTGAAATTATTAGTTTAGAAAAGGATTTGT
CTTTTGAATTATCTTCTGATTATGGAGATGCCGAAACATATTACGAATTAGTAAAATCTA
TCGATAAAGCTGAAAATGATTTAGAAACATATTTAGAAAATTTAACTAAGGACTAAAATG
GCGAGTTTAATTTTTACTTATGCAGCAATGAATGCTGGAAAATCTGCTTCTCTTTTAACT
GCTGCACATAATTATAAAGAACGCGGAATGAGTGTATTAGTTCTTAAGCCTGCTATTGAT
ACTCGCGATTCTGTCTGTGAAGTTGTTTCTCGCATTGGAATTAAACAGGAAGCGAATATT
ATTACAGATGATATGGATATTTTCGAGTTCTATAAATGGGCTGAAGCACAAAAAGATATT
CATTGCGTATTTGTAGATGAAGCCCAATTTTTAAAAACTGAACAAGTACATCAATTGAGC
CGAATTGTTGATACATATAATGTTCCTGTTATGGCTTATGGATTAAGGACTGATTTCGCT
GGAAAATTGTTTGAAGGTTCTAAAGAACTTTTAGCGATTGCAGATAAACTTATTGAACTA
AAGGCAGTTTGTCATTGTGGTAAAAAAGCGATTATGACAGCTCGATTAATGGAAGATGGA
ACACCAGTTAAAGAAGGTAATCAAATCTGTATTGGTGATGAAATTTATGTTTCTTTGTGT
AGAAAACATTGGAATGAATTAACTAAAAAGCTTGGTTAGTGCAAAAGTTATAAATAGGTT
TATCTAACTAAAGGGGTATATATGCTACAATTAACTGAAAAGCAACTTCGCAATCTTACT
GTGCTTCAATTAGATGAAATTCGTAGGGAAGTTGGAAATATCATTTCAGCTTTGCGTCGA
GAAGTATCACTCAACCAATCTCCGGCAGACTATACTAGATTGCGAAATTTTGAAAAATAC
CTTGATAAAGTTAAGGCCGTGCACCGGCATAAAGTAAATACAGGACAAAAATGATAGGAG
GCCTTTATGGCCTTAAAAGCAACGGCACTATTTGCCATGCTAGGATTGTCATTTGTTTTA
TCTCCATCGATTGAAGCGAATGTCGATCCTCATTTTGATAAATTTATGGAATCTGGTATT
AGGCACGTTTATATGCTTTTTGAAAATAAAACTATAGAATCGTCTGAACAATTCTATAGT
TTTATGAGAACGACCTATAAAAATGACCCGTGCTCTTCTGATTTTGAATGTATAGAGCGA
GGCGCGGAGATGGCACAATCATACGCTAGAATTATGAACATTAAATTGGAGACTGAATGA
AATTCAGCGACTTTTCACAAAGTGGAAAACCTTCAAAGGCAGATGAATACTTAGGTTTAT
TAATGGCTGCACAAGCTTATTTTCATTCTGCACATTTTGAAACTAAAAGTTATGCTAGAC
ACAAAGCATACGATTTTATTTTCTCCGAGTTGCCAGATTTGATTGATAAATTTGGTGAGC
AATATTTGGGGTATTCTGGTAGAAAATACACGCCTTCAATTCCAGATGCCAGTAAACTTC
CTACCGACACAATTAAAATGATTGATCGCATACTAGACCAATCTAACAGCATTTATAAAG
AAATGCCTCCAGCCATTCAAAGCACGATAGATGATATTACTGGAATGTTTTACCAGAGTA
AATATCTTCTTTCCCTCGAATAACGTTAGTCTCCTTCGGGAGACTTTTTTCATTTTACCG
GTTTACTTTCCATTTGAGCTGTGATACTATACAACCATCGGATAAAGAGGAGAACATCAT
GAAAATTGAAGCACTTAATCAAGAAGGAAATATCTACGTCATCATTAATGGTGATTTTTT
CGTCGACATGGATGAAGTTACTAGTGAAGAACTTGTAGAACTTCTTAAGAAACGTTATAA
TATGTGTGATGAAGTTGCAACTCACATGGCGTGTGCAATATTCTCTCTTTCATATGTGGT
GGAATAATGATTAGTATCGAACAAGCGGATAAAATTAAGGAATTGGTAGCTTTAATTCGT
AAAGCAGATGAGGAACTTAGTGACTTTGCTTGGTTTTCATCAGGCATTGCAAATAAAGGC
ATTGATGAACTTGAAGCTAAAGTTGATAATGCTTTAGAAGCGTTAGATATGTTTCTTGAT
GAAATTATCGATCATAATACGAGAGTTTAAGTATGCTAACACGTGAACAGTTTGAAAAAA
TCATTAAATTAGCACGTGATATTGAAATAGATTCATACCAATTAGCAGTTGAGCATTGTG
AAGGATATTCATACGACGGTATAGAAGCAGCTAAAAAGGATTTGGATAAATCCAAAGCTA
AGTTAGTTCAATATCTTGAAATGATTAGGTGGAATAATGAAAACTGAAAAGCAGATGTTT
TTAATGAAGCTAATTGAAGAATATGCTAATGCAGTTTCTGACTATGAGTATTCTGCTCGG
GAAAGAGGTACGGCTTTCGCTAAAGAAGAATTGAAAATCATGGTTGATGCTCATACAAAG
CTTCAGAATTTTATCGAAAACGTCATTTAATGGTTTACAAGTTGGCAAGGTTATGGTATA
GTAATCTTGTCGACTGCCAAGGAGAAGAGAATGAAAGTTTTGTTTGTTGTGTATGTGATG
ATTCAATATAATTACCCAATGTTTACTTATAATTTGGTAAACAACATTATTGATATGATT
CAGAGGAGTATGTAATGACAAGTGAGCAGGCTTTTAAATTAAGAGAATTAATTGAGACGT
ATAGTAAAGCTGTTCATACATCAACAATTATTGATGAATCAGCTTTCTCCGGACATGCTA
ACAAGATTAAATACAAAACTCTTATGGAAGAAGCTAAAGTAAATCTTGATTCTTATATTG
AAACTTTAATTGGTAAATAATATGAAAGCTGTAATTGAAACAACTGATTTGTTCGGTGAC
CTAGTTATCGAAAAACGCGGTAATGTATACGTTCTGACGCAGGAAGACGATAGTATTACT
ATTCTTCCTATGGAACTGGCTGCGATTTTATCTTATGCTCCCCCAGGAGTTACCGGTGTA
ACTAATATTACAGGTGACCTCCAAGTTCGTTTTTATCGTGGCCTTTACGGCGGTAATATT
GAAACTGAATTTATGTGTCTTTCTATCAACAATTGGGCAACATTCGTTGCCAAGGTGAAA
GAATTCTTAGAATCTGAAACAGCCGAAACACCCGAAAAGGCTAAACTTCAATGGGCTAAA
GATCGCGGTGCTAACATTATTAATCCAGTAGCTGCAAACTATACTACGACTTTGGATGTC
AAGACTTCTTATGAAGATGGCGATGTCGTACTTGTTCGCCAAAATGATGATTCTCGAGCA
CATATTGTCACATTTACCAAAGACGAAGCTATTGCGCTAAAGACTTATCTTGATTCTGTC
ATTCCAACTATGATTTCAAAGTGAGGAAATATGTTTATTTCAAGTGGAAGTGGTTTAATT
CGTGTTGAATTTAAAAATGACATCTTTCTTATTCAAGGAGATGATATTATTAAAATGAGT
TATGACGAAATCAAGAAAATTTGTCATGCTCTTGAAAGTCATGGAAAAGTGAACGCTGTT
ATTGATATTGGCGATTTATGGGTGACACTTTATGAAGTATCCGAAGGATTTAACATTGAA
GATGAAAATAACATTTTAGCTATTGATAAAAGAAGTGATTTGTTTGATGTATTAAAAGTT
TATGAACAGTCAAATGGCGGAAGAAAAGCTGCATTGATTTATCAAAAACCGCATTCATGT
GGAACTGCTTCAATCATTTCAAATATTGAAGATGAAGTTGATACTTATATGTGTGTTTTA
AAAGCTGGTGGTGACCGTCATCCGGATTTTATTTCTATTCGTCAAAACAATGGAGAAATT
TCATTATCAAAATCAGAAGCTGAAGCTATGATTAAGTATTTAACAACTGTTACGCCTTCA
ATGAAAGGATAATTATGATTATTAATGAAAACTCTTGGCACTATAAATTATTCAAATTGT
TTAACGATGAATGGCAACGACCTAAGACACTATGCGCATATTTTTGGTCTATTGCCATTC
CTACATTTTTCGTTTCTATTTTTGGATGTGCTATACTCGTAGGGCTAACAATTATTTGTG
CAGAAAGCCTACAACGTTGGCTTATTTTCGGTAGTTTATGGACTCTTCTTCCATCAGCAT
TTATACTTGCGCTTTTGGTTGTTTTACTTATTATCGGTTCATTTGTTATTCCTGCACATT
TGCATGAAAAATATAAAGATTATAAATGGAAAAAGGATTATGCTTTACACATAGAAAATA
TTGATAGGGCGTATAAAGGTTTACCTCCTATTCAACCCAAGAAATCGGTTATCGTTGAAT
TTTTAAAAGCTCGTAAAGCTAAAGTATGTCCTGTTATTGAATATAAGGCTGAATGATGAA
AACAGTAATGAAAAGCTATTTTGGTAGTCATCTTTATGGAACTTCTACCCCAGAATCTGA
TGTAGATTTTAAAGAAATTTTTGTTCCTCCTGCTCGTGATATTCTTATCGGAAATGTCAA
AGAGCACATGAGTAAAAACACTAACAACACATCATCTAAAAACACTAAAGATGATATTGA
CCATGAACTATACAGTCTTAAATATTTCTTTAAATTAGCAGCAGATGGTGAAACTGTAGC
ATTGGATATGCTTCACACTCCACCTGAACTAGTAGTTAAATCAGATTTGCCTGATGTGTG
GAAGTTTATTCAAGACAACCGTTCTCGTTTTTATACGACTAACATGAAATCCTATTTAGG
ATATGTCCGCAAGCAAGCTTCTAAATACGGTGTCAAGGGTTCTCGTTTGGCTGCATTACG
TGATGTATTGAAAGTAGTTAATCAAATCCCTGAGCAATGGGTTGATTACCAAGAAGACGG
TTCCATTAAGCAGCGTCGCACTAAAGTTGAAGATATTAAGCATCGTCTTCTAGAAAACGA
ATTCTGTGAATGGGTGTTCCATAATCATGAGAAAACAGGTCCACAAACGTTCTATACTGT
ATTGGGTCGTAAATATCAGACAACGCTTTCTCTTATTGAGCTTAAGCAGTCACTGAACAA
ATTAGATGCTGAATATGGTGAACGTGCTCGTAAGGCCGAAGCCAATGAAGGTATTGACTG
GAAAGCTCTGAGTCATGCTTGTCGTGGTGGACTTCAACTATTGGAAATTTACAAAACTGG
TGACTTGGTTTATCCACTCCAAGATGCTCCATTTATTCTCGACGTGAAGTTGGGTAAACA
TCCATTTAAAACAGTTCAAGAGTTTTTGGAAGATGTGGTCGATCAAGTAGAAGCAGCATC
TACTGACGCTTCTAAGAATGGTATGCCTAAAACAGTAGACATGGGTTTCTGGGATGACTT
CCTTGAGAAGGTCTATCTTGAAAACCATCGAAGTTATTATAAATGATAGGGAGCCTTCGG
GCTCCCTTTTTTATTTCAAAAATTTTTCACAAAGTTGTTTACAAGCATAAAGCTTTATGG
TACTATACAACTATCAACTGATACGGATTTGGAGAATAAAATGAAAACTGTAACTATCAA
TAAGGGTATCTACTTCGGTAAAGAAATCTCTGGAACTTTTGAGCTCTTAGGCGAATGGTT
CCCAGATAATGCTCCAGTAGATGCGCAAGGAGATGGTAAAGTTTTTGTTGAAATTGACGG
TAAGCGTCGCGGTGTTTGGGTTTACAAATCAGACATTTCATATGATGGTGTAAAAGTTGA
AGAAGTTAAAGAATCGTATGAAGATATGAAAACCCGCATTAATAAAAGATTTAATGTTAT
GGGAATGATGACGAATGGTATTATTAATGGAAATATTCGTTCATTAATTATCTCTGGCGC
GGCAGGTATAGGTAAAACATATTCTTTAGATAAAGCTTTAAATAAAGCAAATGATATTGG
ATACATTGAATATAAAAGCATTAATGGTAAAATCTCTGGTATCGGTCTTTATGAACAGCT
TTGGAATAATCGTGAAGAGAATTCTGTCCTTTTGATTGATGATGTGGATGTTTTCTCTGA
TATGGACATTCTTAATCTTCTGAAAGCTGCTCTGGACACTGGAGAGACCCGTAAAGTCTG
CTGGAGCACCGCATCTTCTTACTTAGAAGAAAAAGGCATTGAGCGTGAGTTTGAATTTAA
AGGAACTATTGTTTTTATCACAAACGTTGACATTGACCGCGAATTAGACCGTGGTACTAA
ACTTGCTCCACATTTACAAGCATTAGTGTCCCGCTCAGTTTATTTGGATTTGGGTGTTCA
CACTAATGAAGAAATTATGGTCAGGGTTGAAGATGTTATTCTTTCAACTGACATGATGCA
AAAGCGCGGTCTTTCTGATGAAGAAACTTATAAAGCATTATCATGGATGAAAGTCAATGT
TAATCGTTTACGCAATGTTTCACTGCGTACTGCTCTTTATCTTGCTGACTTTATTATGAC
AGACAAAAACGGTTGGGAAGAAATTGCTGAGGTTACTCTTCTGAAATAATTCATAAGAGG
ATTTCTATGACAAAAAGGCAGTTCAGAAATAGATTATATGGACTGCCATTAAAAAGATGA
CTAGAATTAAACTGGTAAATGGAGGTAATGATGTTATACTCAAAGGCTCGTGAAATTTAC
GAAACTAAAATTAAAGAAGCAGTATTTCAGTTCGCAACAACGATGCGATGGACAAATGAT
TGGGAGTATTCAAAAAATCATAAGAAGCCCTTGGTGACAAGAAAGGCTCATATGTTAGTG
TTAATAGACCGTGAGCAGATTAAAGCCCGAGAAGCCCTCCAGAATCATAAAAAGGCTGCC
TTTGAATGGTTTATGGATAACATTGCTCCTGAGACTAAGAAAGCAGTGAGCGCGTGGTTC
AGTGGAAAAAATTGTGAAAGAAGTTTCTTTTAGTTGTTTACAAGACCGTTCCTCTGTGGT
ACTATACAACTATCAACTACGGAGGAACAGAAAATGAATGCTAAAGATATTTTCAACTTG
GTAAACTACAATGATGGTAAATTTAAATCTGAAGCTCAAAGCAAGTTCTTTAATGACATC
TCAATTGGAGGTGAAATCACGGTTGATGGAGGACAAATTTACAAATCTCGTTGGAATTGG
ATCGTTATTATCGATGAGATTGGTATTGTAGAAATTTACAAAAATACGAATAAAAATCGT
ACATTACACTGGTCTCGTGATACTAACGAACAGTACAAAAAGGATAAAGCATCTAAATTA
TCTCGTGTAACTCAAGAAGATATTGAGTTCATCAAGAAAGATATTTTGATGTATGATAAC
TTAATTGTTGAAGAGCAAGCTGTTATTGATAAATTTGACGAGATTAAAGCTTCTCGTGAA
ATTCCTGATTTTATGAAAGAATCAGTAAATGAACGATACACTCTCATTTCAGAGCGTATT
GAAACTTACAAAAAGCAAAGAGCTGAACGCCAGAATACTCTTCGGAAGTTTGAAGAACGG
TTAAAGACGGTACTCGCATAACCGCTTTATACCAAGGATGGTATAATGGTTCTAAGCCCT
TTTAATTGAGATTATTATGAAACAGTTGATAATTAAAAGATTGAATTTATTGATATGTTG
TTTATGTATAGTAATTGCATATGGTTATTACGCAATTAATGATTATATGCATTATAAAGA
TTATGATGTTACTGTAGTTAATACCCTTACAGGAACTCAAGGAAAGGGGTCTAGTTTATC
GTTTATTGCCGTATATGAACTCAAAGACGGTTATAGATTTAGTGAATATATTTCCCCAGA
GATGTATTCATCAATAGAAAAAGGCGATAATATTACTGTAAGTTTACGTCCTTTCGACGT
AAAACAGACATTGTTTGATAATATTGTTTGGTTCTTTGGAATGGTATTAGTTCAATCTGT
ATGCGGTGCTTATATAGTCTTTTCCATCATATTCTGCGTATTTAGTAAAATTGAAATTGA
GTGAGGAAAATATGTCAGTAGTAATTAATAATGTCAATGCAGTAATTAAATCTTTAGTTA
ATAAAAATTGAATGAATGGACTGTACTTCGTCGTGGAGAGCCAGATAAATTTTTTCATAG
ATTTAACCCAACTTTGGATTTGAATGTTATTGACAGAGATGTTCATGCAGAAATTTTAGA
TAAGTTTAAAGTTGATATTGGATTTGGATTAGAAAAACATTTACAGCGAACAAACGGGTC
TGGAATGGGTTTATCTAATCGCATTATGAAAGCCCTTAATAAAATTGGAGCATTGTCTCG
TATTAATGCAAGTGAAATTCTTCGCAATTATAATAAAGGCTATGATCTTTACGGTCGACT
AATGCCGAAATTATCATTTGACCAAATGATCGCAGATTTGTGGGAAAATCAACGACGATT
ATTAGCATTAGGCGCTCGATTAGCTAAAGGTCTAGATAAACAAATGATTTTTAAGACCAA
TAATACAGAAGACCTTAAATGCTTTAAATTTAGTATTCGCGAAGATGATTATTACATCAG
AGCTCGCTCTACAGATTATGTTAATATGGGACATCATCTCTGTTTAGCTTTTGAAGTTTT
AAAAGAAGCCGGAACATTAGAATATTCGTCTGGTGCAAAATGCCCGATTGGTTCAAGTTG
TATTTTAATTTATCGCCCGGATGAATCCAGTTCATCTAAATTACCTACAAAACCTGTACC
AGTTCGTAGTAACGAAAAACATTCTGAACAAATTGATTATTTTAATAAGCAAATTGAAGA
GCTGAATATTTCTATTCAACAGTATGACGATGAAATTTTCAGACTATCTGGATTGAGTAG
TAAAGCTAAATCTGAGCGTGAAAAATTAATTAAAATTGTTGATTTACTTAAATCTTAAGG
AACACCATGAAAACTCGTTCTCAAATTGAAGATATGGTTCGTAATGCCAGCTATACTCGT
GATGTTATGACATTTTTGTGCCAAAATAATTTAGACCCTGATAAAGTTAATCGTGATATT
CACTTTAAGTATATGAATAGCAGTGAATGGTTACGTCATTTTAGTAAAGCGGGATATATT
ACACAAATGACTGCTCGTGAGCAGCTCACCGATTTCTGTAAAACTATTGATTATAAAAAT
CCTCTATTTGTTCAAGGCGTTGGTCAAAGTAAGGTCGATTTATCATCTGGATTTTTCAAT
CCAAATCATTATCGTATTGAATGGAGATTTATTGCTCTATTCCGTAGACAATTAAAGCAA
ATTTTGTCTACTGCTAGTCGATTAAAAGGTTCTGATATTAACTTAAAGAATCTTAAATTT
GATGGTTATACTCTTCAGATGGAAGTAAGACCATTAAAGGAAAATAATAGAACTGCACGA
ATTAGCTTTAAGCCTAATACTAAAAATTCTCTTTCAATTTGTGAATGCCTTAAATCACAG
TTGACAGAAGCATTTAAGTATATGGATGTTGTTGCTGCTGTTCAATCTAAGATTTTACCT
CGTTTTGAACGATTTAAATTAGACACAACAACATATGAACTTGATATGGTTGTTTCATTT
AAATATGATTTTTTGAGAAAGGACGAAGTCCCGCAAGAGAAAAAGCAGGAAGTGCAAGAT
AACTTAAATTTATCCAATTACTTATCAAACGATCCTAAATTTTGGATGTATAGCTCAAGC
AATATAGATGCATGTAAACTTAATAAAGTGAGTTTTCTTCCTACTGAAAATTCAAATTTT
AAACCTGTAGAAAAATGGCACGCTGACGCGATTGAGAAGTCTCTTAAGGCAGTAGATGAT
GAACTCGTTAAAGCGACCAATGAAGTGCTAGAAGCTGAAAAGGCGTTAGAACAAGCTCAG
TCAAGGGTTCAAAATCTGACAAAGCAACGTTCTAAACTGAACAATGCACTAAATGCACTG
AACTAGTTTACTTTGCCACAAGGATGTGGTATAATGTTCTTACTTTCTACTGAGGAGATT
AATATGACTCGTAACGAATATATCAAATCATTCAATAGCGTTATTGATGATAAAGCTATA
CCGATGTTTGGTCAAAATATCGTTCTTTCTATTATCAATCAATGGCTCAATAGTGTTGAT
GCAAGTATTGTTTCTTCTACTAAATTTATTCATGAAATTCGTAAAATTTCTAGCCGTGTA
GATAAAGATGTTATCAAGAAAACCTTTAAAGAGTCTCGTCTTCTTTCATATTTGGTTAAT
CGGGATATTCTTGGTAATTTTGGGAAAGAAATTAAACGAACTAAAGATGTAGTAGGATAT
AATTGGTTCGGTGATGTTAATTCTTATCATCTTAATAATAAAGAAGACCCTGAGAATATT
TTTACTCGTCGTTGGATTAGTAATTTCAGACTTTTCAAAGGACAAATTCTAAAATCAGCT
TCTAAATTATGTTATGGCGATTATCGTCAAATTCATCCTTTGGCTTCTGATATGATTATC
GTAAAAGAATATGAGCTTGATAAAAATAAAGTAGCTATTTTTGTGAATTATGGATTTTTT
ACACCAGAAACTAACCAAAAGAATATTAATAAATTTTTCTCAATTGCTAGCACTATAACT
CGTCAGTTAGAGACTGCATTACTTTGTATGGAAACAGTAGAAAATATTCATACATATCCT
TTTAAGAATATATGCGGATGGGAAGGATATAAAATTGTAATTAGTCTTCGTGAAGTGAAA
TGTGCTTATTCACCTACTAGTAAAGAAATTTACCAACAAAAATGTGATGAAATTGTGAAT
ACTCCTAAAGAAGAAACTACCCTTGAGGAACTAATGGAATGTCTTGATGATTCACCTGAA
CCGGCAGAAATTCGTCCAGAAGTTATTGCACTAGAAAAAGCTTATAAAGAAGTTCTAGAA
ATTTCTAATAAAGCGCAGAAAGAATATGAGCAGGCTAAAAAGATTTGGGAAGAATCTGTC
AATCGCTTGGATCGTCTTGAACAAGCTTTACAGTTAATTAAGTAATTTAAAGCCAAGGAT
GGCTCGGAGTATAAATCATTAACCAAGTGAGAAGAACATGAAAACTCGTAAGCATTATAT
TGATTATTTTGACAGTCTTATTACTAAACACCGTAATTATCAAATCGGACACAGAGCAGT
AATCAATAATATTCTTCGTGATTTTCTTCAATATGTTGGCCAGGAAAATCATATCTGTAA
AGATACTCAAAACGCTTATTCTTATTCGGTAGGCAATTTGCTTGAATGGTTTAAACGTTC
TCGTATGTTATCTTCTACTGTAGCTCGTGATAACATTGAAAACTTTATGAAGCCGAGCTT
CATTAAATCTGCGACATCTATAACCGATTTGGTTGAATTTACTATTGTTAACGATGTTAA
AAAGACCCATTTGGCTGATTGGTTATCTACCATTCCTGAAACTAAATTTGCTGATAAGTT
CGCTTGTCAATTCAATGACCAAGTGAATATGCTTTTTAAGCATGCTCGTAAACTATTTAC
CGCGGGCGATGACCGTACAAATACAGTTCATGTTAAGGACTGGGTTATTGCTGATGAAGT
AACACACAAGCCAGGTGGTTCATCAGTATTAATTAATATCCAGGTTCCTTATTATTACTC
ACGTAATCTTGGCACCATGACAGCTAAAGAAATTAATAAGCACAACAAAACTATTCGTTC
GTTGTCTTATAAACTCCGCATGATGTTAAAAATCATGGACGTAGTGGAAATGTATGACGA
AACCGAAGATAATGGTTCAATGTTATACAGTTCACGCATTCTAATTAAACTGAAGAACCC
TAATACATATAAGACAGTTGTAAAAGAGCCTAAAGTAGAAAAGGTTGATAATCTGAGTGA
AGAACGTGAATATCTCAATGCCCGTTTGATTGAAGTTGAAGCCCAAATTGCAGAACACAC
CAAATTGTTAAAAGCTCTTAATGCAAAAGCAAATGGTTTACGTAATGCTATTGAGGTATT
GAAATGAAAAAGCGTTTATTAGAAGACATTGCAGCTTCAAGTAATTCCAGTTTAATTAAA
ATTATTATGGCTGGTGAGGAAGATGATATGGAAATGCGTGGAAAGATTCACGGCTGCGAC
GATTTAGATTTTAAACCTCCAGCATGGGATGCTATTATGGCTATGGTTGAACGACGTGAA
AGAGCTTCTAAAAACGTTCCTAATTGCCCTGAATGCGGTACTGAACAGGTGCAATTGGTC
CATTGGCAGACTAGTAATCTTCGTTATAAATGTCGCCATTGTAAACACCGATTTGACCGA
GAAGAAAATGACCAAGCGTAAAGAATATATGGAGGCTGCTGAAAAGGCAGTCCGTGAATT
AGCAATAGCTTATTATAATGAACATGGTAAATTTCCTGATAGATACAGCGTGCTTAAATC
TGCTTTAACTCGTTCATATAAAAATATGCTATCAGAAGTAAGTGATATTATATACAAACA
TAAAGAACAAACGGGCCAAAGTCTTGATTACGACGAGACTTTTAAACAAGTACTAGGAAT
TAAGGAATAATATGTTTAAAGTATATGGTTATGATAGCAACATTCATAAATGTGTGTATT
GCGATAATGCAAAACGTCTTTTGACCGTGAAGAAACAGCCGTTTGAATTTATCAACATTA
TGCCGGAAAAAGGTGTTTTTGATGATGAGAAAATTGCTGAGCTTCTGACTAAACTAGGTC
GTGATACTCAAATCGGCTTGACGATGCCTCAAGTATTTGCTCCTGATGGAAGTCATATTG
GTGGATTTGACCAACTGCGAGAATATTTTAAATGATGCTCGAAGGAACTGACTATATTCA
CGATTACCGCGGAAGCGCGGTATATGTAGGTGATGAAGTTGCAGTTTATTATGGGTATGG
AACTTTGATGACAGCCAAGGTTATTCAAATTAAAAATAATCGTGCTAAACTCGAAGTTTA
TTATTCTAATGGTGAAAAGTCTATTTCTAAGTGGAAATACGGCGATTGTATGGTTAAATT
GGGGTAAATATGATTTACGATATTAATGTATCACGAGCTCCGTCAATGGTTACTATTCCT
GCTGAAGAACTAGATCGTCTTCATAAAATTGAAGAACTTCTTTGGGAAATTGAATGTGCC
CTGCCATCAGGACTAGAATCCTGGATTGATGATGAAGAACTTAATAAATTGCGAAATTAA
TTGGTGAAAAATGACTATTGAAAATAAATTAGATGTTGATGCAGTTCTGAGCGAAATCAT
TGAAGACCATGATGCATTTTCAGAAAACTATGATTTCGATTTTTCCGATTATCTTAAACC
TGTCGAAATAGAAGACTGGGTACAAGACGGCAAATGTCAATATCGTCAGTGTGTTTATTT
TAGTCCAAAACATAATGTACATGTAGCGGTAAATGAATCTCGGTCAGGTTCTTATCATTC
TGACTGGTATTATGCAGTTCCGACTGTTGAACTAGTCGAGCCCCATGAACGTGTAGTAAC
TCAGACAGTACGAGAATGGATTACGCTATAAAACCTTGGTGGGCGGCTAGATGGGAAACT
GTAGAGCCAAAGCCGGAAGAATCGGTTTACACTGATGAAGAAACAATATATAATGAACCA
ACGATAAATGACTTAATTGATATGGAGATGGGACATGATTACAGTAGATAAGTGGTTTAG
AATTAATCGTGCTGATACAGGGCTGTGTAATTACTGGCCGGAACTTAGTGCAGGCACTGT
CTTTAAAGTTCGTGAACTTGCAAAAGAATGTGAAGATGATATAGAGCCTGATACTGGAAT
TATTGAAATTGAACTTTCTGATGGAAAGATTATTAACATCTACGATAAGCCAATTACGTA
TTGGTGCTTGTGGAATACTGAATCAGTAGAAAATGGCGAAATTGAAGAAGTTGTAGATAG
AACTAATCAAGTTGTTCAGAAGCCTAAAGCTGATTTTCAAGGTGAACGTATTTCATACGC
ATTAGCTAAATTAGCTGCACAAGAAAATAACGATGGCTATGAAGGAAATTTGATGCAAGC
TGCGGCAGAATATATTGAATGGCTTGAAACCCAAATTTCTTTTTCTGACCAAAAGATTCG
GCAATATAAGCGATTGAATCAAATGTTTTACAATACCTGAAAATAATAAATACCCTTATC
TATTTAAGGTAAGGGTTTTATTATGTTATTGACTGGCAAATTATATAAAGAAGAAAAACA
GAAATTTTATGATGCACAAAACGGTAAATGCTTAATTTGCCAACGAGAACTAAATCCTGA
CGTTCAAGCTAATCACCTCGACCATGACCATGAATTAAATGGACCAAAAGCAGGAAAAGT
ACGTGGATTGCTTTGTAATCTCTGCAATGCCGCAGAAGGTCAAATGAAGCACAAATTTAA
TCGTTCTGGCTTAAAGGGTCAAGGTGTTGATTATCTTGAATGGTTAGAAAATTTACTTAC
TTATTTAAAATCCGATTACACCCAAAATAATATTCACCCTAACTTTGTTGGAGATAAATC
AAAGGAATTTTCTCGTTTAGGAAAAGAGGAAATGATGGCTGAGATGCTTCAAAGAGGATT
TGAATATAATGAATCTGACACTAAAACACAATTAATAGCTTCATTTAAGAAGCAGCTTAG
AAAGAGTTTAAAATGACAATTGAAAAAGAAATTGAAGGATTGATTCATAAAACTAATAAA
GACCTTTTAAACGAGAATGCTAATAAAGATTCTCGTGTTTTTCCAACTCAACGGGACCTT
ATGGCTGGTATTGTGTCTAAACACATTGCCAAAAATATGGTCCCGTCTTTTATTATGAAA
GCGCATGAAAGCGGAATTATCCATTTCCATGATATTGATTATTCCCCTGCTCTTCCATTT
ACTAATTGCTGTTTAGTAGATTTAAAAGGAATGCTTGAGAACGGATTTAAGCTTGGTAAT
GCACAGATTGAAACTCCTAAATCAATTGGAGTTGCTACTGCAATTATGGCACAAATTACT
GCACAGGTTGCTTCCCATCAATATGGCGGAACGACTTTTGCGAATGTAGATAAAGTACTT
TCTCCTTATGTTAAACGCACCTATGCAAAACATATTGAGGATGCAGAAAAATGGCAAATC
GCTGATGCGTTGAATTATGCTCAATCTAAAACAGAAAAAGATGTATACGATGCATTCCAA
GCTTATGAATATGAAGTAAATACTCTCTTTAGTTCAAACGGACAAACGCCTTTTGTAACA
ATTACATTTGGTACGGGAACTGACTGGACTGAACGAATGATTCAGAAAGCAATTCTGAAA
AATCGTATTAAAGGTCTTGGTCGTGATGGGATAACTCCTATTTTCCCTAAGCTTGTTATG
TTCGTTGAAGAAGGTGTTAATCTTTATAAAGACGATCCGAACTATGATATTAAACAGCTT
GCTCTAGAGTGTGCAAGCAAAAGGATGTATCCTGATATTATTTCAGCTAAGAACAATAAA
GCTATCACTGGTTCATCTGTTCCTGTTTCTCCAATGGGTTGCCGTAGTTTCTTGAGCGTA
TGGAAAGATTCGACTGGTAATGAAATTCTTGATGGACGTAATAATCTTGGTGTTGTAACA
CTGAATCTTCCTCGCATCGCGTTAGATTCTTATATTGGAACACAGTTCAATGAACAGAAA
TTTGTTGAATTATTCAATGAACGAATGGATCTGTGTTTTGAAGCTTTGATGTGTAGAATT
AGTTCCTTAAAAGGAGTTAAAGCGACTGTTGCTCCTATTCTTTACCAAGAAGGTGCATTC
GGGGTTCGTCTTAAACCTGATGACGACATAATTGAGTTATTTAAAAACGGTAGAAGTTCA
GTGTCTTTAGGATACATTGGTATTCATGAATTGAATATTCTTGTCGGTCGTGACATTGGA
CAAGAAATTTTAACTAAAATGAATGCTCGTCTTAAACAGTGGGCTGAAAGAACTGGGTTT
GCTTTTAGTTTGTATTCGACTCCTGCTGAAAACCTTTGTTATCGCTTCTGTAAACTTGAT
ACAGAAAAATATGGAAGTGTAAAAGATGTTACCGATAAAGGATGGTACACTAACAGTTTC
CATGTTTCAGTAGAAGAAAATATTACTCCGTTTGAAAAGATTTCTCGTGAAGCGCCATAT
CATTTCATTGCGACAGGTGGTCACATTTCTTATGTTGAACTTCCTGATATGAAAAATAAC
TTAAAAGGTCTTGAGGCCGTGTGGGATTATGCTGCACAACATTTAGATTATTTTGGTGTT
AATATGCCAGTAGATAAATGTTTTACGTGTGGAAGTACTCATGAAATGACTCCTACTGAA
AACGGATTTGTTTGTTCTATTTGTGGAGAAACTGATCCTAAAAAGATGAATACCATAAGA
AGAACATGTGGTTATTTGGGAAATCCGAACGAGCGCGGATTTAATCTCGGTAAAAATAAA
GAAATCATGCATAGGGTTAAACACCAATGAATTATGATAGATTTTATCCTTGCGATTTTG
TGAATGGCCCTGGTTGCAGGGTCGTTCTTTTCGTTACAGGTTGTTTGCATAAATGCGAAG
GGTGTTATAATAAATCAACATGGAATGCTAGAAATGGTATTCCATTCACTGGAGAGACAC
TAGAGCAATTAATTGAATGTTTGAATAATGATTATATAGAAGGATTGACTATAACCGGAG
GTGACCCTCTCTATCCTGATAACCGAGATGTTATTCATTGCATTGTTCAAACGGTAAAAA
ATCTTTATCCCAGTAAAAGCATTTGGTTGTGGACAGGATATAAGTTTGAAGATATTAAAC
AACTAGAAATGCTTAAATATGTTGATGTTATTATTGATGGGAAGTATGAGAAAAATCTTC
CGACTAAAAAGCTGTGGCGAGGATCAGATAATCAGCGACTTTGGTCAAATACCGATGGGG
TGTGGACACATGATTAAATTGAATTACATTATGGATACTATAAATGATATGATTTTTCAT
TTTGGTCCAGAATTTTATTCGCAATATAGTTTAGTGCTTATCAATGCTTGGTTAATCAAT
TAAGGGTAAAATATGTATAAATTTCGTAAAGGTTTAGCTGATTTTCTTACAACTGTAACG
TTCTTTTTGTTTATGGCAGTTGGAGCTATTTTCCTTATTCCTTTTATTGTTGTATTTTTC
GTTATTAGTTTAATTTCTCCAGAAAAGGGCTTATCTTCTAGTGAGTTCAATAAGCGTCTG
GATAAAATTACTAACAAGCTGAATGCTGCTCTTAGTAAGGAATAGTTGTGAAACAAAATA
AGATTGAAGTCTATGGAATTCCAGATGAAGTAGGTCGTTGTCCTGGATGTCAATCAGTTA
CAAAACTTCTAAAGGAGCTTAATGCTCCTTTTACTTTCTATAAAGTTCTTACAAATAATG
GTAAGATTGAGTATGATCGTCCGCTGATTGTATCTCTTGCTAAACGCGCTGGATTCACAT
CTCTTAACATTCGTTATCCGGTCATTTTCATTAATGATTCTAGACAAAAGAACATTAAAC
ACTTCAAAGAAACTCTCATTTCACTTGGATATGATAAAGATATCATAGAAGACTAAGACG
GGCCCTCTGGGCCTTTCTTTCTCACATTCTGTATATTACCATTCTAAGCTATCGTTCCCT
TCTTATCATTCCCTAAAATAATTTTCACAAAGTTGTTTACAACAAGTTCAAACCGTGGTA
TTATTAACATATGAATTGCCTTTGAGGATTTGATATGGTTGTGGTTGATAAAGAAATTAA
AAAGGGACAATATTATTTTATTAATGGTAATGTTGTTCGTGTTACTTACGTAAATGGTTA
TGATGTTTATTATCTTATACTTAAGTTACATAAACACATGATTTGTGATCGTGCTGTATT
TAGTTCAGTTGCTAAGGAAATTAAACTCCATGGGTAAAACGTATCGTCGTAAAGACTTGA
AAGTTCGTGATTATGACTATTTCGGAAAGCGTAAAGCTCCTGATGGGATAAGTCATAAAG
ATATGGTTGAAAACATTTTTCGTTCAGATAAATGGCGACGTATGCGAGGTGTTGATTCAG
AGGTTAAGAATGAATTGAATCGTCAACTGCGTGGTGAAGTAAGAAAGTTGAAAAAATCAG
TTTACATTGACGATGATTTTGATTATAATACATCTCAACGAGTTGCTAAGCGCAAATCAA
ACGAATGTTATCGTTACAGCTGAGGAAAATATGAATATCAAACGAATGCTTTTTAAGCAG
GGGTTATACACTTTAAATGTTGCTCCAAAAGGCGATACGACCAAGTGGTCAGTAAATGAC
TGGATTAAATTTATTGATGAAAATGGTAATTGGAAAATTTAAATGAATCCTGAATCTAAA
TTATCGCAACGAATTGCTGAAGCTCGTCGCCAGTTCTTTGAAAATATGATAGCCCAGGGT
ATTGATGACGAAGTTTTTCTGAACTGGTTTTGGAATCACAAATATTCAAAATGCGCAGGA
GCAATATTAATATCAGCCGCAATGATGTACGAAGGCTGGAAGGGTGCCAAACAGTTTAGC
TAAGGGCTTCGGCCCTTTTTGGATAATAAAATTTTAACGCAATTGAGGATAATGTATGAC
TATTCAAATTAAAAACGCCATCAATTCTTATGCATATGATAAAGTAGTTTCTTTGCTAGA
AAAAGGCGATATTGTAACTCCTCAAAGTTTGGATAAATGGGAAAAAGAGCTTCATCAGAC
GATGAAACAGAATGATCAGAAGATTGGACGCAACACTATCCGCGAATTGTTGGTTCAATA
TATCTTGTCAGAATTTGATGTTAAAGCTTTTGGCGTAGAATCTAAAGCTTATCAAAAACA
TGAAATTTCCGATAAAACTATTCGTCGTATGAAAAATCAACGCAAGAAAAAATTTGCTGA
CCTGAAAATTACTAAGGTATAATTATGAACGAAGCTCTTATTAACGATTTACGTCTTGCC
GGATATGAAGTAAATACAAATGGCATTGGTTTAACTCAAATTGAAGGAAATGGATTCATC
CTTGAGTATGAATTTAGCCAATGGTGGTTATATGCCAATTATGGCGAATTGATTGAATAT
GTTGACCAATTTGATTCACTAGATGCAGCTCTTGAAGCGGCTAAGTTGATGAATGTATGA
AATTTATTAATATTTCTATTACTATTGAAAATTATGGCATTTTCTATGTTGACCAATACA
TGAAAATTTCATTTTTCCCAAATAAGACTGGTGTTGGATATTGGGAAAGCCATGTTTCTG
AATTAAATGAAAGTGAATATGTTAGTACACATGAAAAGTTTTTAGACTTTTTATATCATG
CTGATCTAACTAGTCATTATATAGATATTCATGAATTTAAAAAGATGATGGAGAAAGTGT
TCCAAGCATACTGTTTACTTAGATAACTGATTCATCCTCTGTGCTTTAAGATAGATCTTC
AAATATTATGATATAATAGATCTATGAATTGAGCTAAGAGGTGAAAATGTCAGAAACTAA
GCCTAAATATAATTACGTAAACAATAAAGAGCTTTTACAAGCTATTATTGATTGGAAAAC
AGAATTAGCAAATAATAAAGACCCAAATAAAGTAGTTCGTCAGAATGATACTATCGGATT
AGCCATTATGCTTATTGCAGAAGGCTTATCTAAGCGTTTCAACTTTTCAGGATACACCCA
GTCTTGGAAACAAGAAATGATTGCAGATGGTATAGAAGCTTCTATTAAGGGGCTTCACAA
TTTTGATGAAACGAAATATAAAAACCCACATGCATATATAACTCAAGCTTGTTTTAATGC
ATTCGTCCAACGTATTAAAAAAGAACGTAAGGAAGTTGCAAAGAAATATAGTTACTTCGT
TCACAATGTCTATGACAGCCGTGACGACGATATGGTTGCGTTAGTAGATGAAACTTTTAT
TCAAGACATCTATGATAAAATGACGCATTACGAAGAATCAACCTATAGAACACCGGGGGC
TGAAAAGAAAAGTGTTGTAGACGATTCTCCTAGTTTGGATTTTTTATATGAGGCTAACGA
TTAACCTCTCCGGATTCTTGGAAGAAATACCTGAAGTTGAAGCTATTCCCTATTTACTTA
AAATGTATCTCAGGGAAGTTTTAGCTCTTGACATTGATATTGATCCAGAAAATCCGTATG
ATACCGCTTTTAAATCTAATGGTGTAGAATTAAACTATCGGTATCATTTAACAGATGATG
ATTTTTATTTTATATTAGAGAAATAATATGACTGATAAACCCGAAATTAATGATGAAGTG
GAAAAGCTTATTTCTTCTATTGAAGAAAAGAACCGTCTTGAAGCAGAAAGAAAAGCAAAT
AAGTTATTGTCTAAAAACAAACGCGAACTGAATCGTCTTTATAAGCACGCTCAGACTGCA
GCTGAAAACAATAATTTTGCTCAATACGAATATGCTATCAAGAAAAGTCGGGATATTCTA
AAACAGCCATATAACGATGAACTCATCAGTATTCTTTGGAAGACTACTAGATCGCAAATT
GAGGATATGATTGATGCTTACACACGTAAAATTCAAGCGTCTTAAAATTAATGCAGGATT
TACTGAATCTTTGAATGGTCATCTTTGTGTGAAAATTTCTGAAAAAGAATACCATGATAG
TTCAATTAAAGAAGTTAATCCTCCTATTGTAAGAGCAGACCCTAATATGAAAGTGTGGGT
TGATTCTTATCAAGTCAAAAAATGGTGGCAGTTATGAAAGATGAACACCCAGACTTCTGA
AATAGATTATAATAAAATTCGTTCCTCTAAAGAGGAAATGATGAGACTCTTTAAAGCGTC
TCATGATAAAGCTAAAGCAGAAGGAACTATAAAATATAAGCGCATAAAATTTAAAAGTTC
TAACGAGCCTCTGTATGGCGTATTATGTGGATAGGAGCTTCGGCTCCTATATTGCTTTAT
AAATTTTTGGTAAAATAAACCAAAACAAAGAGGATATTAAATGAAAGTATGTATTTTTAT
GGCTCGAGGTCTTGAAGGTTGTGGTGTAACTAAATTCTCACTCGAGCAACGTGATTGGTT
TATTAAAAATGGTCATGAAGTGACTTTGGTTTATGCTAAAGATAAATCATTTACTCGTAA
TTGCGCGCATGATTATAAATCATTTTCAATTCCGGTTTTATTGGCAAAAGAATACGATAA
AACACTTAAGCTGGTAAATGATTGTGATATTCTAATTATCAATTCAGTTCCTGCTACTTC
GGTTGAAGAAGACACTATTAATAACTATAAAAAAATTATTGATAACATTAAACCTTCTGT
TCGTGTTGTAGTTTATCAGCATGACCATTCTTCTCTTTCTTTGCGTCGAAATTTGGGATT
AGAAGAAACTGTTCGTCGAGCTGATGTTATTTTTAGCCATTCTGATAATGGTGATTTTAA
TAAAGTTCTGATGAAAGAATGGTATCCTGAAACAGTTTCTCTGTTTGATGATATTGAAGA
AGCACCGACAGTATATAACTTTCAGCCTCCTATGGATATTGCGAAGGTTCGGTCAACCTA
CTGGAAAGATGTTTCTGAAATTAACATGAATATCAACCGTTGGATTGGTCGTACGACCAC
ATGGAAAGGTTTTTATCAGATGTTTGATTTTCATGAAAAACATCTTAAACCTGCAGGACT
AAGTACTATTATGGAAGGTCTGGAACGTTCTCCAGCGTTCATTCCTATTAAAGAAAAAGG
AATTCCATACGAGTATTATCGTCTTCATCAAGTAGACCAAATTAAAATTGCTCCTAATTT
GCCAACGCAAATTCTTGACCGTTATGTAAATAGCGAAATGCTTGAACGTATGAGTAAATC
CGGTTTTGGTTATCAGTTGAGTAAGTTGGATAAAAAATATCTACAACGTTCTTTAGAATA
TACTCATCTCGAGCTTGGTGCATGTGGAACAATTCCGGTATTTTGGAAATCTACTGGCGA
AAATTTAAAATTCCGTGTTGATAATACTCCTTTGACCTCGCATGATAGCGGTATCATTTG
GTTTGATGAAAATGACATGGAATCAACATTTGAACGTATTAAAGAACTGTCATCTGATCG
AACTCTTTATGACCGCGAACGCGAAAAAGCTTATGAATTTTTGTATCAGCATCAAGATTC
AAGCTTCTGCTTTAAAGAACAGTTTGACATTATTACAAAATAAAGGGCTTCGGCCCTTTA
GCTTTATACGGAGTTTGATATAATGATATTTCTTGGATATGTGATACTTTTTCTTGCATT
TTATCTATTCACTAGAGCATGTTGGATTGGGTTCTTTAGCACGCCAGATGGGTTTATTTC
AATAATTTTATTTTGCATTTCAATGACGGTTCTTGATATATGAAAATTTTAAATTTAGGT
GATTGGCATTTAGGCGTTAAAGCCGATGATGAGTGGGTTCAATCCATTCAGTTGGATGGA
ATTAAACAAGCAATAGAATATTCTAAGAAAAATGGAATTACTACATGGATTCAATACGGC
GATATTTTTGATGTGCGAAAAGCAATCACGCATAAAACTATGGAGTTCGCTCGTGAAATA
GTTCAAATGCTTGATGATGCTGGTATTACCCTACATACTGTTGTAGGAAACCATGATATG
CACTTTAAAAATACTTTAACTCCAAATGCCTCTACTGAGCTTTTGGCTAAATATCCTAAT
GTTAAAGTATATGATAAGCCTACTACAGTAGATTTTGACGGATGTTTAATTGATTTAATT
CCTTGGATGTGTGAAGAAAATACTGGTGAAATTCTTGAACACATCAAAACTTCATCTGCT
TCTTTTTGCGTTGGTCACTGGGAACTGAACGGATTTTATTTTTATAAAGGAATGAAATCT
CACGGTCTTGAACCTGATTTCCTTAAGACTTATAAGGAAGTGTGGTCTGGTCACTTCCAT
ACTATCTCTGAGGCTGCTAACGTCAGATATATTGGGACACCATGGACACTAACTGCAGGT
GACGAGAATGACCCTCGTGGGTTCTGGATGTTTGATACAGAAACAGAACGAATGGAATTT
ATTCCAAACAACACTACCTGGCATCGTAGAATTCATTATCCATTTAAAGGAAAAATTGAC
TATAAAGATTTTACAAATCTATCAGTACGTGTTATAGTAACTGAAGTAGACAAAAATCTG
ACGAAGTTCGAATCTGAACTAGAAAAAGTTGTGCATTCATTACGAGTTGTGTCAAAGATT
GATAACTCTGTCGAGTCAGATGACAGTGAAGAAGTTGAAGTTCAATCTCTTCAGACGTTG
ATGGAAGAATACATTAATGCAATTCCAGACATCACTGATTCTGACCGCGAAGCACTTATT
CAATATGCAAATCAGTTATATGTAGAGGCAACACAATGACTTTTGATGAATTTAAAAATG
TTATGATGAGTCAGCATTTTGAATGCGAAGTAAAAGATGATATTGGGCATAAAGAAATTA
TTGAATATTGGTTTGAACCGCTAGAGGTTGAAGATAATTGTATTAAAAAGGTTACGGTCT
GCACTGACTGGGCTGTATCTTTTAACTTCAACATTTTAGATAATGACACACCTAAATCAT
TACGAGATATGGCTGTATCTTGTATTAAGGATGCATACTGTGAAGTTTTCGACATTTGAC
ATTAACGATGAATTCATAGCAAATATTGATTATACCGAAGAAGATTCTAGATATGTTGGA
ATAATTTATATTACATCAAAAACAGCACAAGGTGTTGTTTGCATGGCTGAATTTGATGAA
TACTTTTTAGATTATGATGATATGATAGAATGGTCTAAAAGATACATTAAAAGGAATCTT
TTGTGAAGAATTTTAAACTAAACCGAGTTAGGTATCAAAATATAATGTCAGTAGGTGGAA
ATCCTATTGACATTCAATTAGATAAGGTTCAAAAAACTCTTATTACTGGCCGAAATGGCG
GTGGTAAGTCTACTATGTTAGAAGCCATCACATTTGGGCTTTTTGGCAAGCCATTTCGTG
ATGTAAAGAAAGGTCAATTAATAAACAGCACAAATAAGAAAGAACTTTTAGTTGAACTGT
GGATGGAATATGATGAGAAAAAGTACTATATCAAAAGAGGACAAAAACCAAACGTTTTCG
AAATCACCGTTAACGGTACACGTCTTAATGAATCTGCCAGCAGTAAAGATTTCCAAGCAG
AATTTGAACAGCTTATCGGAATGTCATATGCCAGTTTCAAGCAGATTGTTGTCCTTGGTA
CAGCAGGGTATACCCCTTTCATGGGTTTGTCGACCCCTGCACGAAGAAAGCTTGTGGAAG
ACCTGCTTGAGGTAGGAACATTAGCTGAAATGGATAAGCTTAATAAAGCACTAATATGTG
AATTAAATTCACAAAACCAAGTGCTTGATGTTAAAAAAGATAGTATTATCCAACAAATTA
AAATATATAATGATAATGTTGAACGCCAGAAAAAATTGTCTGGTGACAACCTTACTCGTC
TTCAAAATATGTATGATGATTTGGCAAAAGAAGCTAGAACGCTAAAATCAGAAATAGAAG
AAGCTAATGAAAGATTAGTTAATATTGTTTTAGACGAAGACCCGACTGATGCATTTAATA
AAATCGGTCAAGAAGCAGTTTTAATTAAATCAAAAATTGACTCGTATAATAAAGTCATTA
ATATGTATCATGAAGGTGGATTATGTCCAACCTGCTTGTCACAATTAAGTTCCGGTGATA
AAGTTGTTTCTAAAATTAAAGATAAAGTTTCTGAATGTACGCATTCGTTTGAACAGCTTT
CAACACACCGTGATAATTTAAAAGTTCTTGTTGATGAATACCGAGATAATATTAAAACTC
AGCAGTCGTTGGCAAATGATATTCGCAATAAAAAGCAATCTCTAATCACGACGGTAGATA
AAGCTAAAAAAGTTAAAACAGCTATAGAAAAAGCATCTTCTGAGTTTATTGACCATGCTG
ATGAAATAGCACTGCTTCAAGAAGAACTTGATAAAATTGTTAAGACAAAAACTAATTTAG
TAATGGAAAAATACCACCGAGGAATTTTGACTGATATGCTCAAAGATTCTGGTATTAAAG
GTGCTATTATTAAAAAGTACATTCCATTATTTAATAAACAGATTAACTATTATCTTAAAA
TAATGGAAGCGGATTATGTGTTTACATTAGATGAAGAATTTAATGAGACAATTAAATCCC
GTGGTCGTGAAGATTTTAGTTATGCTTCATTCAGTCAAGGTGAAAAAGCGCGAATTGATA
TTGCTCTTTTATTTACTTGGCGTGATATTGCTGAAAAAGTTTCAGGTGTTAAAATAAACA
CACTAATTCTTGATGAAGTTTTTGATTCAGCGACCGACGTTGAAGGTGTAAAAGCTATTT
CAACTATTTTAGATAGTTTAAAAAATACTAATGTTTTTGTTATTTCGCATAGAGACCATG
ACCCACAAGCATACGGTCAGCATCTTCAAATGAAGAAAGTTGGTCGATTTACTGTAATGG
TTTAATTTATAAGAGATTATGCTTTAATTTATTAGAGTATAATCTCTGTGGAGGAAAAAC
ATGGAATATTCAACTGGACAACATCTATTAACTATTCCTGAAATAAAACGATATATTCTG
AGAAATAATTTTTCTAATGAAGAGCATATAGTTACTGAATCTATGCTTAGGAATGCATTT
AAAGCAGAATATACAAAAATAATGTCCAATAGAAATGAAGCTTGGACTGTTACTGATTAT
TATGACTAAAGGTGTATTATGACTAAAATTACTGTGAATTATACTGTTGACGTAAAAGAT
ATTCAGCCAAAACACGTGCGTTCTGAATCAAATCCACAAAACCAAAATAAAATTCGTCGA
GCATGGGTTTTGTCTCTTTCTGATAACGCAATGGAAGTTATTCAGAACAAAATTAAATCT
GCACCTGCTCGTCATGCGTATTATGAAGCTATCGATCATGAAGTAAGTAATAAATGGATT
GAACTAATGCGCAAACATACTACAGAATCTCTAAACGCCGGTGCTAAATTTATTATGACT
TCATGTGGTGAACGCCTTGAAGATGAATATTGCGGTAATGCAGATGAACGTCTAATTGTT
GCTGCTCAAATTGTTGCCGAAACAATTGCGGCTGATTTTAATCGTTAATTGCTTTATTAA
ATTAGTTATAAAATTAAATCTCATTTGAATTGAAGGAAATTACATGAAACTGTCTAAAGA
TACTACTGCTCTGCTTAAAAATTTCGCTACTATTAACTCTGGTATTATGCTTAAATCAGG
TCAATTTATTATGACTCGTGCAGTTAATGGTACAACTTATGCGGAAGCAAATATTTCTGA
CGTTATTGACTTTGATGTAGCAATTTACGATTTGAACGGTTTTCTTGGTATTCTGTCTCT
AGTTAATGATGATGCAGAAATTTCCCAGTCAGAAGATGGAAATATTAAAATTGCTGATGC
TCGTTCAACAATTTTTTGGCCAGCAGCCGATCCGAGTACAGTAGTTGCTCCTAATAAACC
AATTCCATTCCCGGTAGCATCTGTTGTTACTGAAATTAAAGCTGAAGACCTTCAACAACT
GTTGCGTATATCTCGTGGTCTGCAAATTGATACAATTGCTATCACGGTAAAAGAAGGTAA
AATCATAATTAACGGTTTTAATAAAGTAGAAGATTCTGCTTTGACTCGTGTTAAATATTC
TTTGACTCTTGGTGATTATGATGGTGAAAATACATTTAATTTCATTATCAATATGGCAAA
TATGAAAATGCAACCAGGAAATTATAAACTTCTGCTCTGGGCAAAAGGTAAACAAGGTGC
TGCTAAATTTGAAGGTGAACATGCGAATTATGTAGTAGCTCTTGAAGCTGATTCTACCCA
CGATTTTTAATAGAGGGCTTCGGCCCTTTATAATTTACACTAAAACTTGAATGAGGAAAT
TATGATTACCGTAAATGAAAAAGAACACATTCTTGAACAGAAATATCGTCCATCTACTAT
CGATGAATGTATTCTTCCCGCCTTTGATAAAGAAACCTTTAAATCTATTACAAGTAAAGG
TAAGATTCCACATATTATTCTTCATTCTCCTTCTCCAGGAACAGGTAAAACAACTGTAGC
GAAAGCATTGTGTCATGATGTAAATGCTGATATGATGTTTGTGAATGGATCAGATTGTAA
AATTGATTTCGTTCGTGGTCCTTTGACTAATTTTGCCAGCGCCGCTTCATTTGATGGTCG
TCAAAAAGTAATCGTTATTGATGAATTTGACCGCTCAGGATTAGCAGAGTCTCAGCGACA
TCTTCGTTCCTTTATGGAAGCTTATAGTTCAAACTGTAGTATTATTATTACTGCTAACAA
TATTGATGGTATTATTAAACCGCTTCAGTCACGCTGTCGAGTTATTACGTTCGGTCAACC
GACCGATGAAGATAAAATTGAAATGATGAAGCAGATGATTCGTCGGTTGACCGAAATCTG
TAAGCATGAAGGAATTGCTATAGCTGATATGAAAGTTGTAGCAGCTTTGGTTAAAAAGAA
TTTTCCTGATTTTCGTAAAACTATTGGCGAGCTCGATAGTTATTCATCTAAAGGTGTTTT
GGATGCTGGTATTTTATCACTGGTTACTAACGATCGTGGTGCTATTGATGATGTTCTTGA
GTCTCTCAAAAATAAAGATGTTAAACAACTCAGAGCTTTAGCACCAAAATATGCAGCTGA
TTATTCGTGGTTCGTAGGTAAACTTGCCGAAGAAATCTATACACGCGTAACTCCGCAAAG
TATTATTCGTATGTATGAAATTGTTGGTGAAAATAACCAGTATCATGGAATTGCGGCTAA
TACTGAATTACATTTAGCATATCTTTTCATTCAATTGGCATGTGAAATGCAGTGGAAGTG
ATATGAGCTTATTTGAAGATGATATTCAATTAAACGAGCATCAAGTTGCTTGGTATTCAA
AAGATTGGACAGCTGTCCAATCCGCTGCTGATTCTTTTAAAGAAAAAGCTGAAAATGAGT
TTTTTGAAATAATTGGAGCTATTAATAATAAAACTAAATGCTCTATTGCTCAAAAAGATT
ATTCAAAATTCATGGTTGAAAATGCATTATCACAATTTCCAGAGTGCATGCCAGCTGTAT
ATGCTATGAATTTAATTGGATCAGGCTTAAGTGATGAAGCTCATTTTAATTATCTAATGG
CTGCAGTTCCTCGTGGTAAAAGATATGGTAAATGGGCAAAACTAGTTGAAGATTCCACCG
AAGTATTGATTATTAAGTTAATTGCTAAGCGGTATCAAGTTAATACAAATGATGCAATTA
ACTATAAATCAATTCTTACTAAAAATGGAAAACTTCCTTTAGTATTAAAAGAACTAAAAG
GTTTAGTCACGGATGATTTTTTGAAAGAAGTGACTAAGAACGTAAAAGAACAGAAACAAC
TCAAAAAACTAGCATTGGAATGGTAAAATGATTGAAATTACTCTTAAAAAACCTGAAGAT
TTTCTGAAAGTAAAAGAAACTTTGACTCGTATGGGAATTGCTAATAATAAAGATAAAGTT
CTGTATCAGTCCTGTCATATTCTTCAGAAAAAAGGACTATACTATATCGTTCATTTTAAA
GAAATGCTTCGTATGGATGGTCGTCAAGTTGAAATGACAGAAGAAGATGAAGTTCGTCGT
GATTCGATTGCATGGTTGTTAGAAGATTGGGGATTGATTGAAATCGTTCCTGGTCAAAGA
ACTTTTATGAAAGATTTAACTAATAACTTCCGAGTTATTTCTTTTAAACAAAAACATGAA
TGGAAACTCGTTCCTAAATATACGATTGGTAATTAAGGTAAATTATATGACACAATCAGA
GATTCAGTTTACCGAAGCTTTTCTAATATTCGAAAATCTAAAACAAACATATGGTCTGTC
TTCTACCCAACTGGTTTTTGACCTCCCGGAAGATAAACAAATAGAATTCCAAAAAGAATT
CCAACGATTAGTTTACCCACGCCAACGCGCATTTCATGCGATTATCAAAACCACAAATAC
AGATGGCAAGTCCGTTATATCGCGTCGTACAATAGAAATTTAAAGCAAAGGGGCTTCGGC
CCCTTATTTGGAGTATAATATATCAAGAGCCTAATAACTCGGGCTATAAACTAAGGAATA
TCTATGAAAGAATTTTATATCTCTATTGAAACAGTCGGAAATAACATTGTTGAACGTTAT
ATTGATGAAAACGGAAAGGAACGTACTCGTGAAGTAGAATATCTTCCAACTATGTTTAGA
CACTGTAAGGAAGAATCAAAATACAAAGACATTTACGGTAAAAACTGCGCTCCTCAAAAA
TTTCCATCAATGAAAGATGCTCGAGATTGGATGAAACGAATGGAAGACATCGGTCTCGAA
GCTCTCGGTATGAACGATTTTAAACTCGCTTATATCAGTGATACATACGGTTCAGAAATT
GTTTATGACCGAAAATTTGTTCGTGTAGCTAACTGTGACATTGAGGTTACTGGTGATAAA
TTTCCTGACCCAATGAAAGCTGAATATGAAATTGATGCTATCACTCATTACGATTCAATT
GATGATCGTTTTTATGTTTTCGACCTTTTGAATTCAATGTACGGTTCAGTATCAAAATGG
GATGCAAAGTTAGCTGCTAAGCTTGACTGTGAAGGTGGTGATGAAGTTCCTCAAGAAATT
CTTGACCGAGTAATTTATATGCCATTCGACAATGAGCGTGATATGCTCATGGAATATATT
AATCTTTGGGAACAGAAACGACCTGCTATTTTTACTGGTTGGAATATTGAGGGATTTGAC
GTTCCGTATATCATGAATCGCGTTAAAATGGTTCTCGGTGAACGTAGTATGAAACGCTTC
TCTCCAATCGGTCGAGTAAAATCTAAACTTATCCAAAATATGTACGGTAGCAAAGAAATT
TATTCTATTGATGGCGTATCTATTCTTGATTATTTAGATTTGTATAAGAAATTCGCATTT
ACTAATTTGCCGTCATTTTCTTTGGAATCAGTTGCTCAGCATGAAACCAAAAAAGGTAAA
TTACCATACGACGGTCCTATTAATAAACTTCGTGAGACTAATCATCAACGATACATTAGT
TATAACATCATTGACGTAGAATCAGTTCAAGCAATTGATAAAATTCGCGGGTTTATCGAT
CTAGTTTTAAGTATGTCTTATTACGCTAAAATGCCTTTTTCTGGTGTAATGAGTCCTATT
AAAACCTGGGATGCTATTATTTTTAACTCATTGAAAGGTGAACATAAGGTTATTCCTCAA
CAAGGTTCGCATGTTAAACAGAGTTTTCCGGGTGCATTTGTGTTTGAACCTAAACCAATT
GCTCGTCGATATATTATGAGTTTTGACTTGACGTCTCTGTATCCGAGCATTATTCGTCAG
GTTAACATTAGTCCTGAGACTATTCGTGGTCAGTTTAAAGTTCATCCAATTCATGAATAT
ATCGCAGGAACAGCTCCTAAACCGAGTGATGAATATTCTTGTTCTCCGAATGGATGGATG
TACGATAAGCATCAAGAAGGTATCATTCCAAAGGAAATCGCTAAAGTATTTTTCCAGCGT
AAAGATTGGAAAAAGAAAATGTTCGCTGAAGAAATGAATGCCGAAGCTATTAAAAAGATT
ATTATGAAAGGCGCAGGGTCTTGTTCAACTAAACCTGAAGTTGAACGATATGTTAAGTTC
AGTGATGATTTCTTAAATGAACTATCGAATTATACTGAATCTGTTCTCAATAGTTTGATT
GAAGAATGTGAAAAAGCGGCTACACTTGCTAACACAAATCAGCTGAACCGTAAAATTCTT
ATTAACAGTCTTTATGGTGCTCTTGGTAATATTCATTTCCGTTACTATGATTTGCGAAAT
GCTACTGCTATCACAATTTTCGGCCAAGTTGGTATTCAGTGGATTGCTCGTAAAATTAAT
GAATATCTGAATAAAGTATGCGGAACTAATGGCGAAGATTTCATTGCAGCAGGTGATACT
GATTCGGTATATGTTTGTGTAGATAAAGTTATTGAAAAAGTTGGTCTTGACCGATTCAAA
GAGCAGAACGATTTAGTTGAATTCATGAATCAGTTCGGTAAGAAAAAAATGGAACCGATG
ATTGATGTTGCATATCGTGAGTTATGTGATTATATGAATAACCGCGAGCATCTGATGCAT
ATGGACCGTGAAGCTATTTCTTGCCCTCCGCTTGGTTCAAAGGGCGTTGGCGGATTTTGG
AAAGCGAAAAAGCGTTATGCTCTGAACGTATATGATATGGAAGATAAGCGATTTGCTGAA
CCGCATCTAAAAATCATGGGTATGGAAACTCAGCAGAGTTCAACGCCTAAAGCGGTCCAA
GAAGCTCTCGAAGAAAGTATTCGTCGTATTCTTCAGGAAGGTGAAGAGTCTGTCCAAGAA
TATTACAAGAACTTCGAGAAAGAATATCGTCAACTTGACTATAAAGTTATTGCTGAAGTA
AAAACTGCGAACGATATAGCGAAATATGATGATAAAGGTTGGCCAGGATTTAAATGTCCG
TTCCATATTCGTGGTGTGCTAACTTATCGTCGAGCTGTTAGCGGTTTAGGTGTAGCTCCA
ATTTTGGATGGAAATAAAGTAATGGTTCTTCCATTGCGTGAAGGAAATCCATTTGGTGAC
AAATGCATTGCTTGGCCGTCGGGTACAGAACTTCCAAAAGAAATTCGTTCTGACGTACTG
TCTTGGATTGACTACTCAACTTTGTTCCAAAAATCGTTTGTTAAACCGCTTGCAGGCATG
TGTGAATCGGCTGGTATGGACTATGAAGAAAAAGCTTCGTTAGACTTCCTGTTTGGCTGA
TAGAATAAATCTAGGGACCTCCGGGTCCCTTTTTCATACAAGTAATATAAATCTATACTT
ATGAAAAACAGATGATTCTGGACCTTTAGAATTCCCTAAAAAAATTTCACAAAACTGTTT
ACAAGACTGTTCTTCTGTGGTACTATACAACTATCAACTACTGATACAGATTTGGAGAAT
AAAATGAAAATTGTTATGGGATGCTTAGCAGTTTGTTTAGTTGCATTAGTAGCAGTTCCA
TTTGTTAGTGTTGAAAATGATACTCAACCTGTGATTAAATCTAGCACCGTTATTCACACT
AATGGTAAAATATCAGTTAAAATTGATGATAATCTTCATGTGAATACAAATGGATCTTTA
GGTGTGCAAATGGGTAATACCTGCATAAGCACTACTGGAGTAATTACTACTTGCATTTGA
GGAAATTATTATGAAAATCGCTATTTTGGTTATTGCATTAGGTCTTACTGGTTGTGTGGC
TCAAGGACCGGTAGTAAATCAGTCTGATGTAGGAAAAATTGTAAACTGTTCAAGCAAATT
TTATAATCCTAATGTCAAGTGTTATAAAGAAGCTCCAAAGCAAACAGTAGAACAAATGCA
GGCGAATTTTGACGAAGCTATTCGTCCAGATGAATCTGCTCAAGCATATCGTAATTCGGA
TGTAATTACACGCGAAGAAAAAATTGAAAACTACTGCGCTGAGCTTTGGGCTAATTGGGC
TAATAATTACCAATGGCGTACTGGTAAAAATGCTCCGATGGAGTATGTAGTGAATTCTTA
TAATTCATGTGTAAAAAATTTGACTAAGTGAGGAAAACATGGAAACTTTAGTAGCAGGTT
CAATTTTTATGGTTTTAGTTTCAGGTGTGTTGGCTATTATTATATACATGCTTCCATGGT
TTATCGCCTTGATGCGTGGGTCAAAATCGACAGTAGGAATCTTTTTCACATCTTTACTGT
TTAACTGGTCAATTATTGGTTGGTTTATTACATTTATTTGGTCAATTGCGGGTGAAACTA
AAAAGTCTGCACAACCAAATCAGGTAATTATCATCAGAGAGAAGGAATGAAAAGCAAAAT
TATAACAGTGTTGCTTTTAATCTTGATGATTATAATAAGTATATACTATAGTGTAACGGT
TCCTCTTATGATTCCAACTATTATTTTAGGTTGGGGTTTATTACTGTTACAAGTTAAATA
TGAATGTATCAATTGAGGTTTAAATGATTAGTGACTCTATGACAGTTGAAGAAATCCGTC
TTCATTTGGGGCTTGCATTAAAGGAAAAAGATTTCGTAGTTGATAAAACTGGTGTTAAAA
CTATTGAAATTATTGGCGCATCATTTGTAGCAGATGAACCGTTTATTTTTGGCGCTCTTA
ATGATGAATACATTCAGCGTGAACTTGAATGGTATAAATCTAAGAGCTTATTTGTTAAAG
ATATTCCAGGCGAAACACCAAAGATTTGGCAGCAGGTAGCATCTTCTAAAGGCGAAATTA
ACTCGAATTATGGTTGGGCTATTTGGTCAGAAGATAACTATGCCCAGTATGATATGTGTT
TAGCTGAACTTGGTCAAAATCCTGATTCTCGACGTGGTATCATGATTTATACTCGTCCAT
CCATGCAATTTGACTACAATAAAGATGGTATGTCAGATTTCATGTGTACTAATACAGTAC
AGTACCTGATTCGTGATAAGAAAGTCAATGCAGTAGTAAGCATGCGCTCAAATGACGTGG
TCTTCGGATTCCGTAATGATTATGCATGGCAAAAATACGTATTAGATAAATTAGTATCTG
ATTTGAATGCCGGCGATTCAACTCGTCAGTATAAAGCAGGTTCTATTATATGGAATGTTG
GAAGTCTTCATGTATATTCTCGTCATTTTTACTTGGTTGATCATTGGTGGAATACTGGCG
AAACTCATATTGTGAAAAAAGATTATAACGGTGAGTGGAAATGATTCAATTCGTAATTCC
AAGTTATCAGCGTGTCGGGGCAGTTTCTGCCCTTGATATGTTTCCGACTGATTATGAACC
CCATATTGTAGTGCGTGAACATGAAGAAAAAGCTTATCATGACGCTTATGGGTCTAGAGC
TAAAATTATAACTATTCCTGATGATGTAAACGGAATTGCCGGTACTCGTAAAGCAATTAC
TGATATGTATGCAGGTCAACGAATCTGGATGATTGACGATGATACTACTATTCGTATGAG
TTCAATGCGAAAAAGAGATGACCGTCGTTGTGTAGATAAAGTCAATCAATTAACCCGTGA
ACAGTTCTATGAATTGATTCAATACGTTGAAGATGCCATGGATTGCGGGTATTATCATGG
TCATGCTCGCCTACCAATTTTTAAAATTACTTCATCTTGGGGTAATTATCGTGAAAATTC
ATATGGATTCACGAATACATGGTATGACCTCGGAAAACTTACTACAGAACAAATTGGGTA
TGGAAAAATTGATTTGTGCGAAGATATGTATGCATTTCTCAATTTAATTAATCAAGGTTA
TCCGCATTTGGCCTTGTTCAAATATCTGGTTGTATCAGGTAAAGCGCAAGCTCCTGGTGG
GTGTAGTTCAATTCGTAGTAATTCTAAACATAATAGAGCGCTTGAGCAAATCAATAGAGA
GTTTCCAGAGCAAGCTCGTTGGAAAACGTCGAATATTGAAAAACGAAAATCGTTGGGTGA
AGAAGACGAGCCATTAAAGGTTCTTCGTATGTGTGTTTCGCGCAAAGAAAAATCAGAAGC
ATTTCATAAGTTTAATGCTATTCATCCAATAGCAGTTGATTAATGCCTAAATTTATTGTG
TTATAATTACTCTATCTTTAACCAGTGAGGAAAATATAATGATGCCTATGGAAAAAATGA
ATGTCTATTGCAGATTTAAAATCCCGTTTGATTAAAGCTTCCACTTCTAAAATGACTGCT
GAACTGACTACATCTAAATTCTTTAATGAAAAGGATGTAATTCGTACAAAAATTCCAATG
CTTAATATTGCTATTTCTGGTGCGATTGATGGCGGTATGCAGTCTGGTTTAACTATTTTC
GCAGGGCCTTCTAAACACTTTAAATCAAATATGTCTTTGACTATGGTTGCGGCATATTTG
AACAAATATCCTGACGCGGTTTGTCTATTCTATGATAGCGAATTTGGTATTACTCCAGCT
TATTTGCGATCCATGGGAGTTGACCCGGAACGAGTAATTCATACGCCGATTCAGTCAGTT
GAACAGCTGAAAATTGATATGGTGAACCAGCTTGAAGCTATTGAGCGCGGTGAAAAAGTC
ATTGTATTCATCGACTCAATTGGTAATATGGCTTCTAAGAAAGAAACGGAAGATGCCTTG
AATGAAAAATCTGTGGCAGATATGACTCGTGCTAAATCACTGAAGTCATTATTCCGTATT
GTTACTCCTTACTTTAGCATTAAAAATATTCCATGCGTTGCGGTTAACCATACAATTGAA
ACAATTGAAATGTTTAGTAAAACCGTGATGACAGGTGGTACAGGCGTAATGTATTCGGCT
GATACTGTATTCATTATCGGTAAGCGCCAGATTAAAGATGGTTCTGATCTTCAGGGGTAT
CAATTTGTTCTAAATGTAGAAAAATCTCGTACCGTTAAAGAAAAAAGTAAATTCTTTATT
GATGTTAAATTTGACGGTGGCATCGATCCTTATTCTGGATTGTTAGATATGGCTCTTGAA
TTAGGATTTGTGGTAAAACCTAAAAATGGCTGGTATGCTCGTGAATTTCTTGACGAAGAA
ACCGGTGAGATGATTCGCGAAGAAAAATCTTGGCGTGCAAAAGATACCAACTGTACTACA
TTCTGGGGTCCTTTATTTAAGCATCAACCATTCCGAGATGCTATTAAACGCGCTTATCAG
TTAGGTGCTATTGATAGTAATGAAATTGTTGAAGCTGAAGTTGATGAATTGATTAACTCA
AAGGTTGAAAAATTTAAATCTCCAGAAAGTAAAAGTAAATCAGCAGCTGATTTAGAAACT
GACCTAGATCAGTTAAGTGATATGGAAGAATTTAATGAATAAAGATGATTTGGATTTAGA
TCTAGAAATTATCGACGAATCCCCCTCTTCGGAGGGGGAAGAAGAAAGAAAAGAACGTCT
TTTTAATGAATCTCTTAAGATAATTAAATCTGCCATGGAAAATGTTATCCAGGAGATTGT
CATTAAACTAGAAGACGGTTCTACACACATTGTGTATGTGACAAAATTAGATTGGGTTGA
TGGAAAAGTCGTAATGGACTTTGCTGTTCTTGACCAAGAAAGAAAAGCTGAGTTAGCTCC
TCATGTAGAAAAATGTATTACAATGCAACTACAAGATGCATTTAATAAAAGGTCAAAGAA
AAAATTTAAATTCTTTTAAGGAGTAAGTGTGGTAGAAATTATTCTTTCTCATCTCATATT
TGATCAAGCTTATTTTTCAAAAGTTTGGCCATATATGGATTCAGAATATTTTGAAAGTGG
TCCAGCTAAAAATACATTCAAATTAATTAAATCTCATGTTAATGAATACCATAGCGTTCC
ATCTATTAATGCGTTAAATGTTGCATTAGAAAATAGTTCATTTACTGAAACAGAATATTC
TGGTGTAAAAACACTTATTTCAAAACTGGCTGATTCTCCGGAAGACCATAGTTGGTTAAT
AAAAGAAACAGAAAAATATGTTCAGCAAAGGGCGATGTTTAATGCTACGTCTAAAATAAT
CGAAATTCAAACTAATGCTGAGCTTCCTCCGGAAAAACGAAATAAGAAAATGCCAGATGT
AGGTGCTATTCCTGACATCATGCGCCAAGCATTATCAATTTCATTTGATAGTTACGTTGG
TCATGATTGGATGGATGACTACGAAGCACGTTGGCTATCTTATATGAATAAAGCTCGTAA
GGTTCCATTTAAACTCAGAATTCTGAACAAAATTACTAAAGGCGGAGCTGAGACTGGAAC
ACTGAACGTTTTAATGGCTGGCGTTAACGTTGGTAAGTCATTAGGATTGTGTTCATTGGC
AGCAGATTATTTGCAGCTCGGACATAATGTTCTTTACATCTCCATGGAAATGGCAGAAGA
AGTCTGTGCTAAGCGTATTGATGCTAATATGCTTGATGTTTCTCTTGATGACATTGATGA
TGGGCATATTTCTTACGCTGAGTATAAAGGAAAAATGGAAAAATGGCGTGAGAAATCTAC
CCTTGGTCGTTTAATCGTTAAGCAATATCCTACTGGTGGAGCAGACGCTAACACATTCAG
GTCTCTCTTGAATGAATTGAAGCTCAAAAAGAATTTTGTTCCAACAATCATTATTGTCGA
CTATCTGGGTATTTGTAAATCTTGTCGCATCCGAGTTTATTCAGAAAATAGTTACACAAC
TGTTAAAGCTATCGCAGAGGAATTGCGTGCTCTGGCTGTTGAAACCGAAACTGTTCTTTG
GACTGCAGCACAGGTCGGTAAACAAGCTTGGGACTCTTCCGATGTTAACATGAGTGATAT
CGCAGAATCTGCTGGTCTTCCAGCAACAGCTGATTTTATGCTTGCGGTCATTGAAACCGA
GGAGCTAGCAGCTGCTGAACAACAACTCATTAAGCAAATCAAATCACGATATGGCGATAA
GAATAAGTGGAATAAGTTTTTGATGGGTGTTCAAAAGGGAAATCAGAAATGGGTAGAAAT
TGAACAAGATTCTACTCCAACTGAAGTGAGTGAAGTAGCAGGTTCACAACAGATTCAGGC
TGAACAGAATCGCTATCAAAGAAATGAATCCACTCGAGCACAGTTAGATGCTTTGGCAAA
TGAATTAAAATTTTAGTTTACAAGCTGACAAGACTATGGTATAGTAGTCTTGTTGGTTAA
ATGAGGAGATTGTTATGGAATTGGTAAAGGTAGTTTTTATGGGGTGGTTTAAGAATGAAA
GCATGTTTATTAAAGAAACCACAATGATGAAAGATGACGTTCAATGGGCTACTACTCAAT
ATGCTGAAGTTAATAAAGCATTAGTTAAAGCTTTCATTGATGACAAGAAAGTATGTGAAG
TAGATTGCCGAGGATAATATGCATATTGTTTTATTTAAACCTACTCCGTATAACGTCAGG
AAAAATACGCAATTCAAAGCGCTTATTGCGGATACATGGGAATTGGTGTTAGATATTCCG
GCAGAAGAAAGCCCTCCATTTGGTCGAGTGGAATTTATTAAGTTTGCTGTACGTCCTACG
AAACGGCAGATTCGTCAATGCAAAAGATACTTTCGTAAGATCGTTAAGCTAGAGAAACAG
TTTGTAACATGTGATTATGCAGAAGTTTTAAAATAGCTGTTTACTTTTGTTGGAAATTGA
GATACTATATAAACATAAACTACTGAGGAGATTATCATGAAAAAATTTATTTTTGCTACA
ATTTTTACTTTGGCTTCTTGTGCTGCTCAGCCTGCTATGGCAGGTTATGATAAAGATTTG
TGTGAATGGTCTATGACTGCGGATCAGACTGAAGTTGAAACTCAAATTGAAGCGGATATT
ATGAATATCGTTGAGCGCGATCGTCCTGAAATGAAAGCTGAAGTGCAAAAACAACTTAAG
TCTGGTGGTGTAATGCAGTATAATTATGTTCTGTATTGCGATAAAAACTTTAATAACAAA
AATATCGTAACCGAAATCACAGGCGAATAATATGATTTATTATATGCACAAAAATCCACC
GTTTAAATACGGTAAATTTCCTGATGCACAATGTTATAATATAACGCCTGATGAAAATAA
CCGTGGATATCTTATTGGCACAATTTTTGTAATTGTTAAAGATAACGAAATCGTTGCCTG
GGCAGATTTTAAAGGTACAACACATGATGTTAATCCGGTACCTTTCACATATTATAATAT
AATGGATTTGGCATATGATTATAATTGGTTTAATCATGACGCGTTGGCTCATATTGAAGG
TGTAGGATTTGACATCTCTTATTCTAGCTACTCATTGTGTCCTATGAGTCGGGCTCATAA
AGAACCTGAATATTTTTCATTTAGGAAACGTGTAAACTTTAAACGCAACACAGAGTATGT
CGGTGGAATCTTTGTTAAAGATAATCGAATTATTCGTATTAATTATCCTTTGAGCGTTAG
TCAAAAAGATATTGATGTTGATTTAGATTTCACTGAAAATAATATTAATCATATTGCTTC
TGTGTATTTTGGTATCGATCACAAAATTGTAATATGTGGATATGAATTACCATCAGAAAA
ACAGTCTAAAGTAAATGTTTCTATTGATGACCAAATTTTTAATGCTTTTATGAATAAAGG
TTAATATGTATAGTTCTGAATTTTCATATTTAAAAATGGAAAAAATTTCATACGATTTTA
TAGATGAAAAGGTTTATTACAGCTTCCATGAACCGCGTTTTAATAGTGAGGTTGGGTTTA
TTGTAGTAAAAGACAATTTCATTTTAAAAATATATTCAGCATTAAAGGATTTTCATTACG
AAAATATTAACCTAAAATTTAATAAAGAAAACGTTCGTAATTGTGCAGTAACAATTACAG
GAAATAAAGGCACATGTGTTATGCTATCTGATGAAATTAATGATTTGCTAAATGATGCAG
AAAATATTACTGTGCCATCTATTGATGACCAAATTTTTAATGCTTTTATGAATAGAGGTT
AATATGAAAACATATAAAGAATTTATTAAAGAAGATATGGTAGCTGGAGATTCAGGTGGT
AATCCTGAAAATATCTCTACTGGAACAACGTCGGGCGCTGTAGTAAATAAAGGTCCTGAA
CAGATTCCTAAAAAGAAGAAAGAGGAATCTAAAGAAAAAGAAGAGTAAAAATGTCATCAA
TACCTTGGATTGATAATGAGTTTGCGTATCGTGCATTAGCTCATTTACCTAAATTCACAC
AAGTAAATAATAGTTCAACTTTTAAATTGCGATTTAGATGCCCTGTTTGTGGAGATTCAA
AAACCGATCAGAATAAAGCTCGTGGATGGTATTATGGTGATAATAATGAAGGAAATATTC
ATTGTTATAACTGTAACTATCATGCACCAATCGGAATATATTTAAAGGAGTTTGAACCCG
ATTTATATCGTGAGTATATCTTTGAAATAAGAAAAGAAAAAGGTAAAAGTCGTCCAGTAG
AAAAACCTAAAGAACTTCCTAAACAACCTGAGAAGAAAATAATTAAATCTCTTCCGTCAT
GTGTTAGATTAGATAAACTGGCGGAAGACCATCCAATTATAAAATATGTAAAAGCTCGTT
GTATTCCAAAGGATAAATGGAAATATCTTTGGTTTACAACCGAATGGCCTAAATTAGTTA
ATAGCATAGCACCGGGAACATACAAAAAGGAAATTTCTGAGCCTCGTCTTGTTATTCCAA
TTTATAATGCTAATGGAAAAGCTGAGTCTTTTCAAGGACGTGCATTAAAGAAAGATGCTC
CTCAAAAATATATCACCATTAAAGCTTATCCTGAGGCAACAAAAATATATGGAGTCGAAC
GAGTTAAAGATGGCGATGTATATGTTCTAGAAGGACCTATAGATTCACTTTTTATTGAAA
ATGGTATAGCTATTACGGGTGGTCAATTAGACCTAGAAGTTGTTCCATTTAAAGATAGGC
GTGTGTGGGTCTTAGATAATGAACCTCGTCACCCTGACACTATCAAACGAATGACTAAAT
TAGTTGATGCAGGAGAAAGGGTTATGTTTTGGGATAAATCTCCCTGGAAATCAAAAGATG
TTAATGATATGATTAGAAAGGAAGGTGCAACCCCTGAACAAATTATGGAATATATGAAAA
ATAATATTGCCCAGGGGTTGATGGCTAAAATGCGGTTATCTAAATATGCTAAGATTTAAA
TCAACCCAACTAAAGCAAATGCTAAATCTACAAATGTATCAAGAGTAACTACTGGAATAT
TAACACCATGCGCAATTGCAATTGGTGATAAAATAAAGTTCCAGAGTAAAATTCCTACCA
TAGCAGAAATAGTAAAAGCTATACGTTTTTTATTACCTTTTATGGCATTAACAAGTGCCA
TTAATTTTTGTACCATATGTCCTCCTTATTGCTTTATATATTTATTGTATAATTAATCTA
CTAACCCATGAATTGAAAGGAAAAATAATGGCTTACTTTAACGAATGTTCACAACTGATC
GAAGGCGCTGATAAAGCTCAAAATGAATATTGGGATATTCTCGGTGATGAAAAAGACCCG
CTGCAGGTAATGCTCGATATGCAGAAATCTCTGCAAGTTCGTTTAGCTAAAGATAAACCT
GAATATAATCGTCATCCTGATGACTTGGCTACTGCAGGTGAAGTTGTTGATTGGTTGCGT
AATCAGAAAGATTATATTGACGATGAATTCCGTGAACTTCTGACTTCTCTTGGTGGTATG
AGCAATGGTGAAAAAGATGCATCTGCTGTATGGAAACCTTGGAAGTCGCAGCATGGTGAA
CGTCGTGAAACTCTGATTACTGATTTGTCTCCACAAGACCAATTGGAAATTAAATTTGAA
ATGATTGATATTCTCCATTTCGTTCTGAACATGTTCCAAGGTCTTGGTCTTACAGCAGAA
GAAATCTTTAAACTTTATTATCTAAAAAATGCCGAAAACTTTGCTCGTCAAGACCGAGGT
TATTAACAGCTCGTCTTAAAAGCCGCGAGAATTTATTATAAATAATCATGTAATTTAAAT
AAAAGGAGAATTACATGGCTAGTACTCGCGGTTATGTTAATATCAAAACATTTGAGCAGA
AATTAGATGGAAATAAGAAAATTGAAGGAAAGGAAATTTCAGTAGCTTTCCCTCTTTATT
CTGACGTTCACAAAATTTCTGGCGCTCATTACCAGACATTCCCTTCAGAAAAAGCAGCAT
ATACTACTGTATATGAAGAAAATCAACGTGCTGCATGGATTGCTGCAAATGAAGATTTGT
GGAAAGTAACTGGTGAATAAATTCAAGGACTCCTTCGGGAGTCCTTTTTTCATTTTATGG
TTTACTTTCCAAAATGAGTATGGTATAATAAAAATATCTTATAGAGGAGAACACCATGTT
AAATCGTTGGATTAAACCAAATGAAGACCTCCAGAAGGTTCTTGATAAAGCTATCTCTGA
TAAATGGGGTATGAAAAGCTGGGATTGTGATATCGTCACGCATTCATTCATGATGCATGC
AGATGGTTCAGTCGAGTTCAATGCTGAAATGCGATACACTGACTGGGGTGGATTTCAAAG
AGTCGAATTTCAAAGAGGCTTTTTGTAATGTTTATCTTTAATTGGTTTAAAAGTTTCTTT
ACGGATTTTTTCTCTACGACTCCTGGAGAAGGTGTAGTTCCTATTTCAAATGACTACCTT
CCTTTAACTGTAGTTGAATATGTTTATATGGGAGATGGAACAGTAGAAGCAGTCACTATG
ACTTATGAAGAAGCCCAAGAATATTATAAAAATCCTTGGCGCTGGTCAGTACCTACTACA
TCATCTAACGCGCAGTCTAGTTCTGATTCATATGACACTAATGTTCCTGTTCATGTATGG
GCGGGTGATTCATGTGGAAGTTCTTGTGATTCTAGTTGTTCATCTACATCTTGCGATTGA
GGAAAATTATGGAAGCAATTTTGTTTGAAATGTATATTAGCAGTAATAGTATGTCGTTTG
CTAAAGACGTTCCAATTACCGTAGCAGTAATGATTGATAAGGGTTATTGTGACCCAATGT
ATCTCGTAGAAAATTTCGTTTCAATGCCAGTTCCAGAAGATGCTGAAATAAAACTTAAAA
AGATCGGAATTATTGAAACTGTACCAAATATTCCATTTAGAGCAATTGAAGCATTTACTA
AATCCGAATACATTAATGTTAGCGCAGAACAATATAATGATAAACCCTTATCTTTTTATT
CGTACGATTCAATATATAACTGGAAAATAGATAAAGGAAACAAATTTATAATTGCGAGTG
AAGATGCGTTATCATACTTTATTTCTTCTGTATGGAATAGTTTACATCCAAATTTGCTAA
AAATTCATGAATTTGACGGTGTCCATACTATTGTTTTAGGTAAAATAAATGAAAGCTCTC
AAGAAGATTCTTGAATGGTTCAATAGACCAAACTCAATGTATATTGATGATGGTTGGGTT
GAACAAGCAAATAAAGAAATGCAGAACGAGTCAGAAGAATGGATGAAATCAATGATTAGT
GCTGAGAAAGAAAAGAAATTAGAACGCTCAGTGCTTAAATTGATGAGAGACATCTATGGT
GATAAATCATGACCAAAGATATGACGTTAGAAGAGGCAAAGGCTAAAGCAAATGAAGCTT
TGGATTTGCTTCTTAAAATCGGTAGTAAAATGATGGAAGAAAATGAGAAGTATATCCAGG
AAAATAAAATTCCTGACGGTCCATTAGTAGGCAAAAGGAAATCACATGATTGAAGTAGCA
AAATTATATTCAATAGAATTTATGTCTAAAGAAGGTAAATCAGTAAATACACTTGATAAA
AAGTGCTCATTAATTATTCCTTTAGCAGAAAATCCAGATATTTTAATTAAAGATATAAAA
GAAAGAAAATATCCAGAAAATGTTATTCTAATTATAAAGCATACTGAAGATATTTTGCAG
AATACAGATTCACCATTTTCTTCTTCTGAAGCTTTAACTATTAAAGGCTATAAAAGAGCT
CATGAATATGGTCTTTTTGACATGTTTGAAGACGATAAAGTTAAATTAGCGAGTCAACCT
TCTAAAAGTAAAACATTCATTATTGAAGATATTAAAGATATAAATGCATTTGTTAGGATG
GTCTGGGCTCATTTTGATGTTGGACTACGCTGGAGAATGTCCGAAGAAGAAAGAAAGATC
ATTGAAACTAATCGTTATTTTGGTTTTTATCGCTAGGAATTAATATGGATTTATTTGAGA
TGTTAGAAGATAATCATTCTACGAATATCCAGAATGATTCTAGTGATTATAAAAAAGAGT
ACCGTATAGTATTACAGAATTATGGAATTGAAGCCCCAGATGCTCTTCTAGAAGAACTAG
CTTCATACCATCTTGACCCTCCGCCTTGGGCTCCCTGGGCAAAATAATTCAAAAAGTTGT
TTACTTTCCTTCCTAACAATGATATGATAGCTTCTGAAGTATACGGAGGCTATCATGATT
ATTAATCCTGCAGATGTTGAACAGTTATCTATAAAAGCTGAAAGCGTTGATTTTCAATAT
GATATGTATAAAAAAGTCTGTGAAAAATTTACTGACTTTGAGCAGTCTGTTCTTTGGCAA
TGTATGGAAGCCAAAAAGAATGAAGCTCTTCACAGACAGTTGAATAAAATCATTAAAAAG
CATTTAACTAAATCACCTTATCAGTTATATCGTGGTATATCAAAATCGACAAAAGAACTC
ATTAAAGATTTACAAGTTGGAGAAGTGTTTTCAACGAACAGGGTAGATTCATTTACTACT
AGTTTGCACACAGCATGTGGTTTTTCTTATGTTGAGTATTTCACTGAAATAATATTTCGC
TTAAAAACTGATAAAGCTTTTAATTATTCTGACCATATCAGCGATATTATACTTTCTTCT
CCTAATACTGAGTTTAAGTACACGTATGAAGATACTGACGGGTTAGATTCAGAGCGTACT
GATAACTTAATGATGATTGTGCGTGAACAAGAATGGATGATTCCAATTGGAAAGTATAAA
ATAAATTCTATTTCAAAAGAAAAATTACACGATTCATTTGGAACATTTAAAGTTTATGAT
ATTGAGGTAGTTGAATGAAATACTCAGTAATGCAACTAAAAGATTTTAAAATAAAATCAA
TGGATGCATCGGTGCGTGCTTCTATTCGTGAAGAATTGCTTTCTGAAGGGTTTAATTTAT
CTGAAATTGAACTTTTAATTCATTGTATTACCAATAAGCCAGATGACCATACTTGGTTAA
ATGAAATAATCAAATCTCGTTTGGTTCCAAACGATAAACCTCTTTGGAGAGGTGTTCCAG
TCGAGACTAAGCAGGTGTTAAATCAAGGAATTGATATTATTACATTTGATAAAGTCGTAT
CAGCTTCATATGATAAAAATATAGCTCTACATTTTGCTTCCGGTTTAGAGTATAACACAC
AGGTTATTTTTGAATTCAAAGCTCCTATGGTATTCAATTTCCAGGAGTATGCTATAAAAG
CTCTACGTTGTAAAGAATACAATCCAAACTTTAAGTTTCCGGATAGCCATCGTTATCGTA
ATATGGAATTAGTTTCAGATGAACAAGAAGTAATGATACCAGCTGGAAGTGTATTTAGAA
TTGCAGATAGATATGAGTATAAAAAGTATTCAACATACACTATCTATACTCTTGATTTTG
AAGGATTTAATCTATAATGGAAGGACTTAGATTCATTATACCACGAAAGTTTTAAAGCAT
TTTACATAAAGTTGTTTACAAGCTGAAGTAAAAATGATATAGTATAAGTAAGTTAACCGT
CCGTGAGAAAAATATGAAACTGTCTAATAACCAAATTCGTAAAATTAAACGTCGTTTAGA
GCATACTCAGGCATCTGCAAAAAGACGTTCTAAAGATTTTAACTTAGACTTCAATTACAT
TAAGAACATTTTAGATCAAAAAGTTTGTGCTTACTCGGGAGAACCTTTTGATAATCGTAT
TGAAGGAGAGAAATTATCATTAGAACGTTTTGATAATAACATTGGATACATTAAAGGGAA
TGTTATTGCAGTAAAGAAAAAGTATAATACATTTCGTTCTGATTATACTTTAGAAGAGTT
GATTGAAAAGCGTGATTTATTTGCTTTGCGAATTGGTCGTTCATCTGCGAAAAAAGTTCA
TAAACTAAATTTAGATGAAAAGAAATGGGCTAAAATCAAAAAGACTTATAATCAAATTAA
AGCTATACAGAAAAAACGTGAAAATCGAATTGAACACATTTCTCAGCTTTCTAAATCAAA
ACAGACCTCTGACATTAAGTTAAGGATTATAGCACTTAAAGCTCGTATTGATGGTTCTCG
CATAGCAGAAGGCGCTGAAGTTGTTAAACTGAACGTTCTTCTTAAAGGCTCGGATTGGAA
AATTGTGAAAAAGTTGTCAGAAGCAGAAATGCAATATGATATGTGTGATAAAATTATTCA
AGGTGTAGAGCGGTATCAAAACTTGTCTTTTATTGATAAACTTAAACTGAAAAGAGGATA
CCCGCTAAATTGTTCAATTTTTAAACTTATCCGAGGATAATATGGTTTATGTATATGCGA
TAGTTTACCGAGACAAAGACGGATTTACGGCGCCAGTTCCACTTGATGAACATCGTCCTG
CTGTGTTTTTTGAAAGAGAGATTGCTGATAGAGTTTTTACTACTCTCAAAGACCAATATA
AACTAGCTTTAGGTATGGGAATTCCAAGACTAGTTGAGACTCCACGCAAGTTTTGGTTTA
ATAAAATAGAAGTTAAACATGTTAAGCCTGATGTAGATACACAAAGATTATATCAACGAA
TTCTAGATACTGGGCGTATTGTTAGTATTCCAATTGCAGGGACTTTCCGATGACATTTGA
TGATTTGACAGAAGGCCAGAAAAATGCCTTTAACATCGTTATGAGGGCTATTAAAGAAAA
GAAACATCATGTAACTATTAATGGACCTGCTGGTACTGGTAAGACTACTCTTACTAAGTT
CATCATTGAAGCTTTAATATCTACGGGTGAAACTGGTATCATTTTGGCAGCTCCTACACA
TGCAGCTAAAAAGATTCTTTCAAAACTGTCGGGGAAAGAAGCGAGTACTATTCACAGTAT
TCTTAAAATTAACCCAGTAACATATGAAGAAAACGTTCTTTTTGAACAAAAAGAAGTACC
AGATTTAGCTAAATGCAGGGTATTAATCTGCGACGAAGTGTCAATGTATGATAGAAAGCT
CTTCAAAATTTTGCTTTCAACTATCCCACCGTGGTGTACTATAATTGGAATAGGCGATAA
TAAGCAAATTAGACCTGTTGACCCAGGAGAAAATACTGCTTATATCAGTCCATTCTTTAC
ACATAAAGATTTTTATCAGTGTGAACTTACTGAAGTTAAACGCAGTAATGCTCCTATTAT
TGATGTAGCTACTGATGTTCGTAACGGTAAGTGGATTTATGATAAAGTTGTTGACGGGCA
TGGAGTACGTGGATTTACTGGTGATACCGCTTTACGCGATTTTATGGTAAATTATTTTTC
AATCGTCAAATCGCTAGATGATTTGTTTGAAAATCGCGTAATGGCATTTACGAATAAATC
TGTTGACAAGTTAAACAATATTATTCGCAAAAAGATTTTTGAAACTGATAAAGATTTTAT
TGTCGGTGAAATTATTGTAATGCAAGAACCGTTAATTAAAACATATAAAATTGATGGAAA
GCCTGTATCAGAAATTATTTTTAATAATGGACAATTAGTTCGTATTATAGAAGCAGAGTA
TACATCGACGTTTGTTAAAGCTCGTGGTGTTCCTGGAGAATACTTAATTCGTCATTGGGA
TTTAACAGTAGAAACTTACGGCGATGATGAATATTATCGTGAAAAGATTAAAATAATTTC
ATCTGATGAAGAGCTATATAAGTTTAACCTGTTTTTAGGTAAAACGGCAGAAACTTATAA
AAATTGGAATAAAGGCGGAAAGGCTCCGTGGAGTGATTTTTGGGATGCTAAATCACAGTT
TAGTAAAGTGAAAGCACTTCCTGCATCAACATTCCATAAAGCTCAGGGTATGTCTGTAGA
CCGTGCTTTCATTTATACGCCTTGTATTCATTATGCAGATGCTGAATTAGCTCAACAACT
TCTTTATGTTGGTGTTACCCGTGGTCGTTATGATGTGTTTTATGTGTGAGGCTATATGAT
TAACATCAATTCAAAATATTTAAATCGTCTAATAGATGGTATAAGGAAGCATACTAATAA
GCAAGATAATCTTAATGTTATGGTAACAGGAGCTGAGCTCCTTCATAAGCTTTATCTTAT
AAGTGATACTATATTAGCAATTAAACGAATTGAAAAACAATCATATCACAGTAATACAGA
TACGGTAATTACACTAGATGAAAGTGTCTGTAAATTACTAATTAAATTTGAGGAAGCTAT
TCGTGGAAATAACTAAAGATCAGTTTTATCTTCTTCAGGATAAAGTTAGCGAAATTTATG
AAATAGCTTATAGTAAAAATCGCAAAAACGTGAAAATTGAATCAGGCAAGTTAATGCTTC
AATTGGAAGAAATTGAACGAGATTTGATTGCGTTAGAATTCTTTTGTGGTGAAGTAAAAA
CTGTTACAATCAGTGATTACATTTTAGGTGAAATTAGCCATCTTTATAAGGCGGTTATTA
ATGATTGAATTAAGTTGGTGTCAGTTTAAATCTCTTATGACAAATGTTAAAGCTGTCATT
GAGAAAAATTCTGGTCCTGAAAATATTACTATTCGCGAAAAAGCTTTAAAGATAATATAC
AGTCTTGAAGAGATGCAAAAAGATATTGAATCTATGGCAAAATTTATTGATGAACCTATT
AATAAAGTTTATATTCAAGACTATACTGTAGGTCAAATTCGCGATTTAGCAAGGAAAATT
TAATGTTTGATTTTATTATAGATTTTGAAACAATGGGAAGTGGTGAAAAAGCAGCTGTTA
TTGATTTAGCTGTAATTGCTTTTGACCCTAACCCAGAAGTAGTTGAAACATTTGATGAAT
TAGTTTCACGCGGCATTAAAATCAAATTTGATTTAAAAAGCCAAAAAGGACATCGTCTTT
TTACTAAAAGCACTATCGAATGGTGGAAAAATCAGTCTCCTGAAGCTCGAAAAAATATTG
CACCATCAGATGAAGATGTAAGCACCATCGATGGTATTGCAAAATTTAATGATTACATCA
ATGCACATAATATCGATCCTTGGAAATCTCAAGGCTGGTGCCGTGGAATGTCGTTTGATT
TTCCAATTTTAGTCGATCTCATTCGTGATATTCAACGTCTTAATGGTGTTTCTGAGAATG
AGCTTGACACATTTAAGTTAGAACCATGTAAATTCTGGAATCAGCGTGATATTCGTACCA
GAATTGAAGCACTTCTGCTTGTTCGTGATATGACCACGTGTCCTCTTCCAAAAGGAACTT
TAGATGGATTCGTTGCGCATGATTCTATTCATGACTGTGCGAAAGATATTCTGATGATGA
AGTATGCTTTGCGATATGCTATGGGTCTTGAAGATGCTCCGTCAGAGGAAGATTGCGATC
CTCTATCTCTTCCAACAAAACGATAAAAAGTTGTTTACTTCCTCGGTTAGTTGTGGTATT
ATAACACCATAGCTACTGAGGATAATAAAATGAAAATTTATCGTGTTGAATCATCGTTTA
GTATTCTTGATTATGAAGATGCTATAACAATACGTCGAAATCTTTGTGTTCAAATAACGC
CGTACAGGAGTATAATAGATTCATGGAGCGAAGAGTGGCTATTACACGTAGGTTATGACA
GACCTAATTTTATGCATCATAGCGATAATAATAAAAGAATTCCTTTACCACACGAAGATA
AACTATTAGTTAAAAACGCTAATATAGTAATTAATACTAAGTTCAAGAAAGATTATGTTG
GAGTAGAATATCATATTCCAGGATGGTTTATAGGTCTTTATCATTTTGCTTTCGCTAGTG
AATATGATATGATGAGATGGTTCACACGAGAAGAGCGGGAAGAATTATCTTCTAAAGGAT
TTTATCTCGCTGTATACGAAGTTCCTGAAGATGAAGTTATCATTGGTGGGCACCAAGTAA
TGTTCCGCAAATCGCATGCTGAACTCGTAGATTTTATTGAAATGAGATAATTATGAAATT
TAATTATAATCCTGAATACATACCAAATCCGGTAGCTAAACTGATTGATTTTGATGTTGT
AAGCACTTATGTATGCCCTGTTAAACCACTGGAAATTAAGGAACCTATTATGACTACCGC
TATTGAAATCGGCAAAACCTACAAACTGGTTGAACCTAAAATTAAAACTAATGCCTTGAT
TTCTGGTCATAAAACTCTGACTGATGTTTTTGGCGAAGGCGAATTTATTGTTGAAGAATT
TGCCAAAAGTGAGTGGTTTGACAAATCTTACGTCATCCACGGTCGCCGGTTAGATAATAA
CAAAATAAAGAAAAACCTGGTTTATGAAGATGAGTTCATCCTGTTCCAAGAAGTTGAAGA
ACAAGACCCTACAGACCTGTTGTGTGCTGCTGTGTCTATCCGTCGTCCTTTTGATAATCC
TATCTGTGGTTGGGTAACAGACCAGTGGATTGAAGATGGCGTTGAACTTCTGAACGTAGT
TCATGCTGGAGAATACTCTGTAGTTCCTCGTAGCGCAGTGATTAATGTTCGTTTAAGCTA
GTTTACAAACTCTTGGGACCAGAGTATAATGGTCCTGTGGAGTATAAAATCTTTTTAACA
AGTGAGAGATAATTATGAATATTAATATCGGTGAAATTGCTCGTGTATCTGATAAATCCC
GTTCTAAAGCAGCAGGAAAATTGGTCGAAGTTGTAAGCATTCAGCTTAAACACGGTGTTA
AAGATGAAGATTCTGAAGTAAAAGTACGTATCATTGCTAAAGATGGAATGTCTAAGCCCC
AGTTTGGTTATGTTCGATGGAAATTTCTTGAGCCTGCGTTTTTGAAAGCTGTTCCTGCTA
AAGGAATTGAAACGATTGATACTTCGCATGTAGGTGTAGACTTTAAGTGGAAACTTGGTC
AAGCTATCAAGTTCATTGCTCCTTATGAGTTTGAATTTACCGAAGGTGGACAACCACTTC
CTCAGCGTACTCGCGCTATGTGTGCATACATTACTGATCAATGGGTAGAAGATGGCGTTA
AGTTATACAACGCGGTATTTTTAGGAACATACAAAGTCATTCCTGAAAGTTGGATTAAAC
ACTACAGCAAAGCTCGCTATGCATAAAGTTTAAAATTTTTTCATAAAACTATATACATCA
GTAGTTGATTATGGTACTATATCAATATCAACTACTGATATAGAAAATAATTTGGAGAAT
AAAATGGAAAATTTGCCAAAGGTGCTGCTACTCTTCTGAAACGCATCGCTCCAATGTTCA
ATTAACGAGGAAATTGTAATGAAACGTAAAATTGTTCAGAACTGCACTAATGACGAATTT
GAAGATGTGTTATTCGATCCAGATTTGGTAGTAGTTCAAAAGGAACACACTGCTAAGTTT
ACTCACTTGACTTCGGTTTATGTGTATGAAAAGGTTGGGGATAAACAACCAATTTACGGT
GTATTTCGTGAAATCACTGAAGATGGTACAACTTACTGGAAGGAAATTTATTAATGGCTA
TTAAATTTGAAGTTAATAAATGGTATCAGTTTAAAAATAAACAAGCTCAAGAAAATTTTA
TTAAAGACCATACTGATAACGGAATCTATGCACGCCGTTTAGGTATGCATCCTTTTAAAA
TTTTAGATGTGGATGCTCTTGGGCGTCCTATTAAAATTATGTCATTTGCTGGAAATTTAG
TACTATCTTCTGGTAAAGATATTTTGGATGAAGATTTTATTTGGCTTTCATCGAATGAAG
CTGAATTCTTTAATGAAGTTGAAAATCCATACCAGGCAGCTGAAGAGCAGGAAGAATCTG
CACCGATAACTGATCAATCTAAATTCCCTGTAATGAAAGTTACTATTGAAAATGATGAAC
AGGCATGGTCCTTGTATCAGATGTTGAAAGCTCACTTTAAGGAATAATTATGCCGCTTTA
TGATTATAAATGTCAATCAAAAGACTGCGCAAAAGAATACGAAAAAATCAAGAAAATTTC
TGAAAGAGATACTGATGTATGTCCTGATTGTCATCGGATTGCTATTCGGTTAGTCTCTGC
CCCTAAGCATGTGAATGGCGGATTTTACGACTTACTTAAAGGGTAATTATGTTTAAAATC
GGTAAGAAATATCGCATTCGCGAAGGTGAAGAAAAGAAATATCTATTTTCTGCTATTTAT
AGGAATGGTTCTATTAATGCTGTAATATCTACAAGCGAATTTATCGTTGAAGATATGAAA
GGTAATAATGTTACAATGATTAGTACAGCATCTGGAAATGACGGAAAAATTCTTCACAGT
TTTCAGAGTAATGTTCTAATTTATGATGAAGAATTTGACTTCTTCGAAGAAGTTCCCGAA
GGTTTTGCTTTTGAATGCACTATCACTATGAAATCTGGTGACCCTCTTTCTTTTACAGTT
AAAGATGAAGGAAGTCGCTTGAGAATTATTAGTCTTCTTCAAGCCATTAAATTTAAGTGA
AAATTATGAAATATATTAATCGTTCTATCGCAGCATTAGTATTAGCAGTGTCTTTAATAG
GATGTACTGATGCTGATAATGCAACCAAAGTTTTGTCTTCAAGTGGTTTTACTAATATTG
AAATCACTGGATATAATTGGTTCGGTTGCTCTGAAAATGATTTCCAGCATACTGGATTTC
GTGCTATTGGACCTACTGGGCAGAAAGTAGAAGGAACAGTATGTTCTGGACTATTCTTCA
AGGATTCGACTATTCGTTTTAAATAAAAGGCCTTCGGGCCTTTAGCTTTATGATTACCGG
AGTATAATATTCCCGAAACCAAACGAGGATAAGTGATGATTAAGAATGAAATTAAAATTC
TGAGCGATATTGAACATATCAAAAAGCGTAGCGGCATGTATATTGGCTCTTCTGCTAATG
AAATGCATGAGCGCTTTCTGTTTGGTAAATGGGAAAGTGTTCAGTATGTACCTGGTCTTG
TTAAGCTTATTGATGAAATTATCGATAACTCAGTAGATGAAGGTATTCGTACTAAGTTTA
AATTCGCAAATAAAATTAATATTACTATTAAAAACAATCAAGTAACAGTTGAAGATAACG
GTCGCGGTATTCCACAAGCGATGGTCAAAACACCTACTGGTGAAGAAATTCCTGGTCCTG
TTGCTGCCTGGACTATTCCAAAAGCAGGTGGTAACTTCGGTGATGATAAAGAACGCGTCA
CCGGTGGTATGAATGGCGTTGGTTCTAGTTTGACAAACATTTTTTCTGTGATGTTTGTCG
GTGAAACTGGTGATGGTCAAAATAATATTGTAGTTCGTTGTTCAAATGGCATGGAAAATA
AATCATGGGAAACTATTCCAGGAAAATGGAAAGGAACTCGCGTTACTTTCATTCCTGATT
TTATGTCATTTGAAACTAATGAGCTGTCCCAAGTTTATCTTGACATTACACTAGATCGTC
TCCAGACACTTGCAGTAGTTTATCCTGATATTCAATTTACCTTTAATGGTAAAAAGGTTC
AGGGCAATTTTAAGAAATATGCACGCCAATATGATGAGCATGCTATCGTTCAAGAACAAG
AAAATTGCTCTATTGCGGTTGGTCGTTCACCGGATGGTTTTCGTCAATTAACTTATGTGA
ATAACATTCACACTAAGAATGGTGGCCATCACATTGACTGTGTTATGGATGATATTTGTG
AAGACCTTATTCCACAAATCAAACGTAAGTTCAAAATTGATGTGACTAAAGCACGCGTTA
AAGAATGTTTGACTATCGTTATGTTTGTCCGTGATATGAAAAACATGCGATTTGATTCTC
AAACTAAAGAGCGTTTGACTTCTCCATTTGGCGAAATTCGTAGTCATATTCAACTCGACG
CTAAAAAGATTTCACGCGCTATTCTAAATAATGAAGCAATTCTGATGCCGATTATTGAAG
CTGCACTAGCTCGTAAATTGGCGGCGGAAAAGGCAGCAGAAACTAAAGCAGCTAAAAAGG
CTTCTAAAGCTAAGGTTCATAAACATATTAAAGCGAATCTTTGTGGTAAAGATGCTGACA
CTACTCTTTTCTTGACTGAGGGTGATTCGGCTATCGGATATCTTATTGATGTTCGTGATA
AAGAACTTCACGGTGGTTATCCATTGCGCGGTAAAGTTCTCAACAGTTGGGGTATGTCTT
ATGCAGATATGCTTAAAAACAAAGAATTGTTTGATATTTGCGCAATCACTGGATTAGTTC
TCGGTGAAAAAGCTGAAAACTTGAATTATCATAATATTGCTATTATGACTGATGCTGACC
ATGATGGTCTAGGAAGCATTTATCCTTCTCTGCTTGGATTTTTTAGTAATTGGCCAGAAT
TGTTTGAGCAAGGACGAATTCGCTTTGTCAAAACTCCTGTAATCATCGCTCAGGTCGGTA
AAAAACAAGAATGGTTTTATACAGTCGCTGAATATGAGAGTGCCAAAGATGCTCTACCTA
AACATAGCATCCGTTATATTAAAGGACTTGGCTCTTTGGAAAAATCTGAATATCGTGAGA
TGATTCAAAACCCAGTATATGATGTTGTTAAACTTCCTGAGAATTGGAAAGAGCTTTTTG
AAATGCTCATGGGGGATAATGCTGACCTTCGCAAAGAATGGATGAGTCAGTAGTTTACTT
TACCACAAGGATGTGGTATAATTAATTGAGTAAATGAGGATATTGAAATGAAATCATATA
AAGTAAATTTAGAACTTTTTGATAAAGCAGTTCATCGAGAATATAGAATCATTCAACGCT
TTTTCGATATGGGAGAAGCCGAAGAATTTAAAAACCACTTTAAAGATATTAGAGATAAAA
TTCAATCCGACACCGCAACTAAAGATGAGCTACTAGAAGTTGCTGAAGTTATTAAGCGTA
ATATGAATTAATGAGGAAATTATGATTATCACCACTGAAAAAGAAACAATTCTTGGCAAT
GGTTCTAAATCAAAAGCATTTAGCATCACAGCATCTCCTAAAGTATTTAAAATTCTGTCA
TCTGATTTATATACAAACAAGATTCGCGCAGTAGTCCGTGAATTGATTACTAACATGATT
GATGCTCATGCACTTAATGGAAATCCAGAGAAATTTATCATACAAGTTCCTGGACGTTTA
GACCCACGATTTGTTTGTCGAGATTTTGGTCCGGGTATGAGTGATTTTGATATTCAAGGT
GATGATAATTCTCCTGGGTTGTATAATTCATACTTCAGTTCATCTAAAGCTGAATCTAAT
GACTTTATTGGCGGATTTGGTTTAGGTTCTAAATCTCCGTTTAGTTATACTGATACGTTT
AGTATTACTTCTTATCATAAAGGTGAAATTCGTGGTTATGTAGCTTACATGGATGGTGAT
GGTCCACAGATTAAACCTACATTCGTAAAAGAAATGGGTCCAGATGATAAAACTGGTATT
GAAATCGTAGTTCCAGTTGAAGAAAAAGACTTTAGAAACTTTGCTTATGAAGTTTCTTAT
ATCATGCGACCATTCAAAGATTTGGCTATCATTAATGGTCTTGACCGCGAAATTGATTAT
TTTCCGGATTTTGATGACTATTACGGTGTAAATCCAGAAAGATACTGGCCTGATCGTGGT
GGATTATATGCTATCTATGGCGGTATTGTTTATCCTATCGATGGTGTTATTAGAGACCGT
AACTGGCTAAGCATTCGCAATGAAGTGAATTACATTAAGTTTCCAATGGGTTCACTTGAT
ATTGCTCCATCACGCGAGGCTCTTTCACTGGATGATCGTACTCGTAAAAATATTATTGAA
CGAGTTAAAGAACTCAGTGAGAAAGCATTTAATGAAGATGTAAAACGATTTAAAGAATCT
ACATCTCCTCGTCACACGTATCGTGAATTGATGAAGATGGGATATTCTGCTCGAGATTAT
ATGATTAGTAATTCAGTCAAATTCACGACTAAAAATCTGTCATATAAGAAGATGCAAAGC
ATGTTTGAACCTGATAGTAAGTTATGCAATGCAGGAGTTGTGTATGAAGTAAATCTTGAC
CCTCGACTGAAGCGCATTAAGCAAAGTCATGAAACTTCAGCCGTTGCATCAAGTTATCGT
CTGTTTGGTATTAATGCAACAAAAATTAATATCGTTATTGATAATATTAAAAATCGTGTT
AATATTGTCCGTGGATTAGCACATGCGTTAGATGATAGTGAATTTAATAACACTTTGAAT
ATTCATCACAACGAACGCCTTCTGTTTATTAATCCAGAAGTAGAATCGCAGATTGATTTG
CTTCCTGATATTATGGCGATGTTTGAAAGTGATGAAGTTAACATTCATTATTTGTCAGAA
ATTGAAGCTTTAGTTAAAAGCTATATTCCAAAGGTAGTTAAAAGTAAAGCTCCTCGTCCT
AAAGCTGCTACAGCATTTAAGTTTGAAATTAAAGACGGGCGCTGGGAAAAAGAGGAATTA
TTTACGCTCACATCAGAAGCAGATGAAATTACTGGGTATGTAGCGTATATGCATCGTTCT
GATATTTTCTCTATGGATGGTACTACATCTCTTTGTCATCCATCTATGAATATTTTGATT
CGTATGGCTAATCTTATTGGCATTAATGAGTTTTATGTTATTCGTCCGCTTTTGCAGAAA
AAGGTAAAAGAACTCGGTCAGTGCCAATGTATTTTTGAAACTCTGCGTGATTTATATGTA
GATGCTTTTGATGATGTAGATTATGATAAGTACGTAGGTTATTCAAGTTCAGCTCAACGA
TATATTGATAAAATTATCAAGTATCCTGAACTAGATTTTATGATGAAGTACTTCAGTACA
GATGAAGTTTCTGAAGAATATACACGACTCGCTAATATAGTTAGTTCATTGCGCGGTGTA
TATTTCAACGGTGGAAAAGATACCATCGGTCATGATATCTGGACAGTAACTAATCTTTTT
GATGTATTATCAAATAATGCTTCAAAAAACAGTGATAAAATGGTTGCTGAGTTTACCAAG
AAATTCCGTATTGTTTCCGACTTCATCGGATATCGCAACTCTTTAAGTGATGATGAAGTT
TCTCAAATCGCTAAAACTATGAAGGCCCTTGCGGCCTAATAAGGAAAATTATGTACAATA
TTAAATGCCTGACCAAAAACGAACAAGCTGAAATTGTTAAGCTGTATTCAAGTGGTAATT
ACACCCAACAGGAATTGGCTGATTGGCAAGGTGTATCGGTTGACACAATCCGTCGTGTTT
TGAAAAATGCTGAAGAAGCTAAACGCTCTAAAGTTACTATTAGCGGTGATATTACAGTTA
AAGTTAATAGCGATGTAGTTATTGCTCCAGTTGCTAAATCTGACATTATTTGGAATGCAT
CTAAAAAATTCATTTCAATTACTGTTGACGGTGTAACTTATAACGCAACTCCTAATACTC
ATTCAAACTTCCAGGAAATTCTTAATCTGCTTGTAGCGGATAAGTTGGAAGAAGCGGCAC
AAAAAATTAATGTTCGTCGCGCTGTTGAAAAATATATTTCCGGTGATGTTCGAATTGAAG
GCGGAAGCTTGTTCTATCAAAATATCGAATTACGGTCTGGTTTAGTTGATCGTATTCTCG
ATTCGATGGAAAAAGGCGAAAACTTTGAATTTTATTTTCCATTCTTGGAAAATTTGTTGG
AAAACCCAAGCCAAAAAGCGGTATCTCGACTCTTTGATTTCTTGGTAGCAAACGATATTG
AAATTACAGAAGATGGTTACTTCTATGCTTGGAAAGTAGTTCGCAGTAATTACTTTGACT
GTCATTCAAATACTTTTGATAACAGTCCTGGTAAAGTAGTTAAAATGCCACGTACTCGTG
TGAATGACGATGATACACAAACTTGTTCTCGTGGTCTGCATGTGTGTTCTAAATCTTATA
TTCGTCACTTTGGTAGTTCAACCAGTCGAGTTGTAAAAGTTAAAGTGCATCCGCGTGATG
TAGTATCAATTCCGATTGATTACAACGACGCTAAAATGCGTACCTGCCAATACGAAGTAG
TTGAAGACGTTACTGAACAATTCAAATAAGGGCTTCGGCCCTTATCATATTAAGGAAAAT
TATGTTAGGTTATCAAGCACGAGTAAAAGAAGAATACGATCAATTAATGCTCAAAATTAA
TGCATTGAGCAAATTTTTAGAAAGTACAAAGTTTCTAACGATTAATGAAGTTGAGCAAGA
ATTACTGCTTTCACAGTTCATCTCAATGAAATCTTATGCAGATTGTTTAGAAAAAAGAAT
TGCGCAATTCAAATAAATAAGGGGCTTCGGCCCTTTTGTTTTAAGGAAAATTATGATTTA
TTGTATGAATATCGGTGATTCCGACATAAAAGAGATTAAACTGCACGGAAATCATCACGC
TAATATTGTGTACTGCAATAAATTTGAGTTTGGTCACGAAAAACTTGGGTTTCTTTACTG
CAATGATGTTATAGTCATGGACAAAAAGAATCTCGATGAACTTGATCGTAATTCTTTAGA
TGAAAATGATCGAATTTACTATGGTACTCTTAAAGTGTATAACGCTTATCTTAGTGGTAA
AAAGGAAAGTTTAAATGCAGAAAACGAATCCGGGGTTACAGAGACTATTTCAGATTCCGA
CATTTACCCTATCGAACAGTGACTTGACTAGTGAAATGAAGGTCAAAATTGTTGATACTG
CAAGATACTCTTTAAAACAAAACCCAAATCAGGATAAGGCAGAAGTTATCGAAAGATGCC
GTATCGCTGTGTACGCAGAGTTTTTTGTAGCAGATTGGCTGAGCGGGTATGTTAACAAAG
GTCAAGAAGATGTCGATGATCCGTACACATATGCATGGGATGTATTGGCGCATCCAAGAT
ACTGCGGGCTTCGTGTAGAAGTTAAGACACATCAAACTGACTCACGTTGGATTTCGGTGA
CAACAGGATGCAGCGGAGAGTATCCATATGGTTCTGGAATAAATCTAGGGCCCATTCTAA
ATCATCAAGTCGCGGACTGTATAATTATATTCAACACTAAAGAAATTCACCCAGGTGTCA
TCCAGTACACTCCGAAGTTCATCGGTGATAGAGAAGACCTTCGTAAGGTTGTAAGAAAAA
GCAACTACAACGGATGGTATCTTTCCATTTAAAAATTTTCACAAAACGGTTTACATACCA
CAAGGACCGTGGTACTATACAACTATCAACTGATACGGATTTGGAGAGTAAAATGAAATT
TAAATTTTATTATGCTAAGCATAAAATAACTGATGAGTTTGTGTCAGTTGAATATTCTAG
CGATGATGAAGGCATGATTTATACTCGCTTAGGATTATCGCATTGGGAGTCCGATCAGCC
TTATTTTTCAACTCCTGAAGAAATTACACGCTTAGTTAATGGTCATATGAATGACCACTG
GAATGTAATTCTTTCAGATAGTCTTAAAGCGGCTATTAACAGTAATGAAATGAAAATCGC
TGAGATTGAACTATGAGTTCACTATGGTGGTGTTTTGTTTGGTTAATTAGTATTCCAGTA
ATTTGTTTAACATTTACTTTTGTGATGAGGTTATTATGAAAATTTTTAATTCTGTACTTA
TTGCTTGTGCGTGGTGGGTTGCACAAGTTTCGGCAGTAGTGATTGGTATTCACATTTATT
ACGAATATTTTTAAAAAAGTTGTTTACAAGACTGTTCTTCCGTGGTACTATTACCCTATC
AACTACGGAGGAACAGAAAATGAAAAAGATTGTTAAAGCTATATGGAATGTAGTTATAAT
ACTAATAGTTTTGAGTATATTCCCAATCGTTTTAATGATTGATGTATTAAACGCTTACTT
TGGATTTATGTGAGGAAAATATGAAGCGTAAACGCAGTGCTTTTACATTTATTGAATGGT
TTTTCGATAATATTTTTCCGGCTTTATTCATTTTCATGCTGATTTTTGCCTTAGGTTCAG
TTGTAGTTGGAATCTATTTGATGGCGGTAGTTGGAATGGACATTCATCAAAATGGTTTAA
AATCCGTGGTTGAAACAATTTGGAATGGTGTAAAATGATGAATTTGCTGAGCGGTTGGTT
TTATATTCTTATGTTTTACATTGGCGCAAATTTCCCATATTGGATGGGATGGTCAACAAC
TGCGTTTGGATTTTATACTCCTTGAGGTGAATTATGAAAATCTTTAAAGATGTAAAAGTT
GGTGAAATTTTTTGTTTAGATAATGGTAATCAGTTAATTCGTATTTCACCTCTTAAGAGC
ACTAGCGAGAAACCGACAGTTAATGCTACTTTAGCAAATAACAGTAATGAACGTTTCTGT
ATTGAAAATGATACTGAAACTTATACCGTAGAAGAGTTTTGGGAATTGAGCGTCGACTGC
GACGATTAATTTAATGGCCGCGTGTATTCATGCGGCCTTGGAGTAGAAAATAATTTAGAG
GAAATTAATATGAAATATATGACTGCTACTGATCTGAATAATGCGGGCGCTACCGTTATT
GGTACAATCAAGAACGGTGAATGGTTTTTGGGAACTCCGCATAAAGACTTTTTATCTAAA
CCTGGATTTTACTTTTTAGTAAGTAAATTAGATGGTCGTCCATTTAGTAATCCATGTGTG
TCTGCACGATTTTATGTAGGTAATCAGCGTTCTAAGCAAGGATTCAGTGCAGTTCTAAGT
CATATTCGTCAACGTCGGTCTCAGCTTGCACGTACTATTGCAAATAACAATATGGTATAC
ACAGTATTTTATCTGCCTGCTTCTAAGATGAAACCTCTGACGACAGGATTTGGTAAAGGT
CAGTTAGCTTTGGCGTTTACCCGTAATCATCATTCCGAGTATCAAACACTTGAAGAAATG
AACCGTATGTTGGCTGATAACTTTAAATTCGTTTTGCAGGCATATTAATGAGTAATTTCC
ACAACGAACACGTGATGCAGTTCTATCGTAATAATCTTAAAACTAAAGGCGTCTTCGGAC
GCCAGTGAGGAAAATATGAATATTGCAAAATTATTAGGAGTTATTTCATTTATTTGTTGG
ATAGTAGCATGTGTTTTAACTATCTGTATTGATGCCAGCAGTGTGTTTTCGCAAGCTTTA
GCTCAGGGTATGTGTGCATATTTAACATTTGTGTTGTTATCTACTAATGATTAAGAAAAT
CTTGGGCTATTCATTAGCCCTTGCTATTTTATTACGGAATAATGTTCGGATTAATTCAAG
TCGTGCTTTTCATTTCTGATGTTATTATGGCACTACATTCACTAGTATGGTAAATTTATG
CAACTGAATAATCGTGATTTAAAAAGTATCATTGATAATGAAGCATTGGCTTATGCTATG
TACACGGTTGAAAATCGTGCTATCCCAAATATGATTGATGGATTTAAGCCAGTTCAACGA
TTTGTTATTGCTCGAGCTCTTGATTTGGCACGAGGAAATAAAGATAAGTTTCACAAACTC
GCTTCTATTGCAGGTGGTGTAGCAGACCTTGGATATCATCATGGTGAAAACTCTGCACAA
GACGCAGGTGCTTTGATGGCTAATACTTGGAATAATAACTTTCCTCTGTTAGATGGTCAA
GGAAACTTTGGTTCTCGTACCGTTCAAAAAGCAGCGGCAAGTCGTTATATTTTTGCTCGT
GTAAGTAAAAATTTCTATAGCGTATATAAAGATACTGAATATGCTCCGGTACATCAAGAT
AAAGAACACATTCCGCCTGCTTTCTATTTGCCTATTATTCCTACTGTTCTTCTTAATGGT
GTTTCCGGTATTGCAACTGGTTATGCAACTTATATTCTTCCTCATAGTGTTTCTTCTGTC
AAGAAAGCTGTATTGCAAGCTCTTCAAGGAAAGAAAGTAACTAAACCGAAGGTAGAATTT
CCAGAATTTCGCGGTGAAGTCGTTGAAATTGACGGTCAATATGAAATTCATGGAACGTAC
AAGTTTACTTCACGAACTCAAATGCATATCACTGAGATTCCGTATAAGTATGATCGTGAA
ACTTACGTGAGCAAAATTTTAGACCCGCTTGAAGATAAAGGCTTTATTACGTGGGATGAT
GCTTGCGGTGAGCATGGGTTTGGCTTTAAAGTTAAATTCCGTAAAGAATACTCTTTGAGC
GATAACGAAGAAGAACGCCATGCAAAAATTATGAAAGACTTCGGGTTGATTGAGCGTCGT
TCCCAGAATATTACCGTCATTAATGAGAAAGGAAAGCTGCAAGTTTACGATAATGTAGTT
GATTTAATCAAAGACTTCGTTGAAGTTCGTAAAACTTATGTCCAAAAACGAATTGATAAC
AAAATCAAAGAAACTGAATCAGCATTTCGTTTAGCTTTTGCCAAGGCACATTTCATTAAG
AAAGTAATTTCAGGTGAAATTGTTGTACAAGGTAAAACTCGCAAAGAACTGACCGAAGAA
CTTTCTAAAATTGATATGTATTCTTCTTATGTTGATAAACTAGTTGGAATGAACATTTTT
CATATGACTTCCGACGAAGCAAAGAAACTTGCTGAAGAAGCTAAAACCAAAAAAGAAGAA
AACGAATATTGGAAAACTACTGATGTAGTTACAGAATACACCAAAGATTTAGAGGAAATC
AAATGAGTCCATTCATTGGTATCACAAGCGCTGCATTAGTATCTGGTGGCATTTTACTGG
CGGGTTTAGGTGTTGTTCCTGCCGTAGCAGGAGGTCTTCTTGCGTTCGGAATTCAACGTG
TTATCATGACAGTTATCACAGTCATGCAGTAATTTTAGGGAGAGCCTCGGCTCTCCCTTT
TTTATTTTAAAATTTTTTCACAAAACGGTTTACAACCAAAGCATACTGTGGTACTATACA
ACTATCAAATAAATGAACTGAAACAAACAACCCGGAGATACAAAAAAAAAAATGAAATTT
AAAATCGAAAACGAAATCGTTAAAGCTAAAAATGCTCTGACTGCTAACAAACTGGTTGTA
GATGGTATTGAATATGATATCTGTGGAGTTCGTGAAGAAAAACCTGGTGTTCTGACTTTC
TTCACAATGATTTTTAAATTTAAAGGTGACACAGAATTCAAACAGTTTGATTTTGCCCAT
GAAGACGAAATCGAAGTTCGTAATCTGAACATTAAGTAAGTACTTTATTAGAGCTCTTGA
AAAAGAGTGCAAAAAAGTGTTTACTTCTGCTTTAAACATGATACTATAGACCTATCAAAT
AAATGAACTGAAACGGAGATTAAAATGTCTAAAGTAACTTACATCATCAAAGCTTCTAAC
GATGTTCTGAATGAAAAAACTGCTGCGATTTTAATTACCATTGCTAAGAAAGATTTCATT
ACAGCCGCAGAAGTTCGTGAGGTGCATCCAGATTTAGGTAACGCAGTAGTTAATAGTAAT
ATTGGGGTATTGATTAAAAAAGGCCTGGTGGAGAAATCTGGTGATGGATTAATCATTACT
GGTGAAGCTCAGGATATTATTTCAAATGCGGCAACTTTATATGCACAGGAAAATGCTCCT
GAACTACTGAAAAAACGAGCAACTCGTAAAGCTCGTGAGATTACTTCCGATATGGAAGAA
GATAAAGACCTTATGCTAAAACTTTTAGATGAAAATGGATTTGTTCTTAAAAAGGTTGAA
ACTTACCGCAGTAACTATCTTGCTATTTTAGAAAAACGCACTCACGGAATTCGTAATTTT
GAAATTAATAACAATGGAAATATGCGAATTTTTGGATACAAAATGATGGAACACCATATT
CAGAAATTTACTGATATTGGAATGTCATGTAAAATCGCTAAAAACGGCAATGTGTATCTT
GACATTAAACGCTCGGCAGAAAACATTGAAGCAGTAATCACCGTAGCATCTGAACTGTGA
GGAATAAATAATGAACAAGTTAGAAATTGTTAATGAACTTCGTCGTTGTGCAGAACCCAC
TCAAGAGGGGTGGGATATCTGGTATCATGGAGCTTATCTTGGAACTATCGTAAAGATTAA
GACTGGTAAATACATGATTATTCGTGAAAGTAAAGATGCTCCAGTAGGTATTCGCAATAA
TTTTATGGCGGCGATAAGTTCATTTACGGATGCAGCTTACGAAATTTACCTTGCTGATTA
TAAAGAATTCCAGGAATCTCAACCAGTTATTCGCTCAATTGGTGTTAACAAAGCTCAACA
GAAAACTTTGTGGCAGCGTATTAAAGGATGGTTTAAATGAACCCATTTATTAATCGTTTA
AAAATGCTGAATGTTCCTTTATGTCGTGAAACTCCAGAAAGTCTTGTTGAAAAATTTAAA
GCGCATGGTTATAAATGCACAGAAGAAGATATTCTAAAAGAAGTTCCTGAAATCTGTTGG
CAGACCGCATACTGGGATGAAAACCAAAAGTATCAACGACGAATTGTTTGCGCAGCCAAC
CGTTTTAAATTAAAAGATGGACGAACTCTTGTTATTCCAGGTGCTCGTCATTATTCTAAA
GATATGGCAGCAGTTTTAGATGTAGTTAAACCTCAATTAGTTACTCAACAAGTTTGTGAT
GATGACCAAGGGTTTATTGACCAATATAGTAATTATTGGACACGTGAAGAAGCAATGATT
ATTGCAACTTACGCCGGACAAGTACGTATTGAACGCGGTGGTAGTGAAAAAGAACTTTAT
TCTGAGGACCTTTACTAATGAATATTAAAAAGTTTCAAATTGATGGAATTAAGAATCAAA
TCCAGGCACTAGAATATGCCAATAAAATGATGTCAACTAATTGGGGAATTTATGCCAATG
AACCGGCATTTCAGTTCTGTGATATGGAATTCACTAAAAAGCTCGTAGGAAAAAATTATG
TATGTCCATTTAGTTCTCCAGTAAATGGAATGCTAAAACCTGCTTTACGCGATCTTTATA
TTGCGATGAACGAAGAAATGATAAAAGAATTAAAACGTCAACTGAAGGTGATTCAATTTG
GCCAGGGAAATTAATTCAAAATCCGATTATTTTAATTCTCTCAATGATAAAGATAAAAAT
TTAATAAGACATTTTATCGTTGAGATGGGATATACTGACACGCATGATTTAAGAGAACAT
ATATTTGAATGTGGTGTAGCTAAAAAGTTTTCATTCACATGCAAATGTCTAAGAGAGGTA
ATTCAGCACTATGAACAATTTAGTCGCAAAACATAATTTTAATAAAGCTTCTGTCCATAA
AGATAAGAAGAAAGCGTTTAAAGAATCTAATCGCAAACAGAAACATAAGGGGAAGGTCTA
TGATTATTGATTCTCAGTCTGTGGTTCAATACACAATCAAAATTGATATTCTAGAAAAGC
TATACAAGTTTCTACCAAATTTATACCATTCAATTGTCAATGAATTAGTTGAAGAACTAC
ATCTTGAGAATAACGATTTCTTGATTGGAACTTATAAAGACCTCTCAAAAGCAGGATATT
TTTACGTAATTCCGGCTCCAGGAAAAAGTATTGATGATATATTAAAAACTATAATGATTT
ATGTCCATGATTACGAAATTGAAGATTATTTCGAATGAGTCATAATCTTGAAAAGGTAAT
CGAGCATAATGTAGCTCAGGAACGTGAGTCGTTCAAGGAATTCGTAGAAAAAATTTTTGA
AGAAAATAACACAGACCAGTTTACAAATCAAGCGTCTGATGATATTATAACAAAGTCAAC
TAATTGAGTGGTATAGTTAATGAATAAAAACATTGATACAGTTCGTGAAATTATTACTGT
TGCATCTATTTTGATTAAATTTTCCAGAGAAGATATTGTTGAAAATCGTGCTAATTTTAT
TGCATTTCTGAATGAGATTGGAGTAACGCATGAAGGTAGAAAATTAAATCAGAATTCATT
CCGTAAAATTGTTTCTGAATTAACCCAAGAAGATAAGAAAACCCTCATCGACGAATTCAA
CGAGGGTTTTGAGAGTGTATATCGATATTTAGAGATGTATACGAACAAATAATTATTTAG
CCCTTCCTAATATTCTGGCCGCCTGAGCACATATTGATTCAAGGCGGTCATTACTTATAT
GATCATTTCTATACCAGTACATGGTTATCGTTCCAGCATAGATATTATCCAAATTAAAAT
ATGGACAACTGTACATGTAGTTTATTTCGGGAGTAGGCTTTTTAGTTGGTAAAAAAGCAA
ATTTTGAGTCAGAATAATAATGACGTCCATTTAAATGAACTGTATATTCATCCATAGTTT
TATCAACAGGATATCCTCCAAGTGATTTTTCACTTATTGTTGAAGGTAATTTTCCTTCGT
ATGCTATAATATCAACAAAATAGTTTAAGTTTTTAGGGCGGAAAGAATACACCGCACTAA
AGTCTGCCTCAGATGATATATGAACTATCTGGAGTTGTTCCAGGGCGACAGATTCAAAGC
GTGCATTTCTTTCCTTTTCAATAATTTCACTGTATGTTTCATACTTTGATTGCTTATAGT
ACTCAAAGAAACTATCTCCCCTATACCAAACAATCGCCATCATAAATAAAAGAATTACGA
CAGCTACCCGGGAAGCAAGAACTTTCCCGGTAGCGTTATCTTTGAACAAGCGATCTAGAA
CACCAAACAGAATATCAGAGGGCGAAAATGATATTCTAGGTGCTGCCATAGACCCTCCTT
TTAAGGATATTTATTCAAAATTATACCCTTGGACCATATATTACTCCAACATTTTGCCAT
GTTGGAGCTGATCCTATAACAGCATATCCGGCAGCGCCACCAGCTCCAACTTTTAGAACT
TTACCGCCATCATACCAGGCATTTTGTCCATTTGCGCCAGCATCACCACCCTGGCCAGAG
TGAGCATTACCATAATTATAACCACCTCCTGGGCCATCATAAGAAGCTGCGCCGCCGGAA
TAACCTCCGCCTCCTCCTGGGCCAAATGGTCTTCCTCCACCACCACCAATATCTTGCCAA
TTTGCACCAGAGTGAGGACGATAAGCGATACCACCTCCACCTCCACCTCCACCACAAATT
ACTCCACCGTTATTAATTCTTAATCGTCCACCGATGTCATTTTGAATACAATGACCACCT
TGTTGACCATTACCATCTCCGCCGGAATGAGTCCATCCACCTTGTCCACCTCTACCCCAC
ATATGGACGCCGCCACCAATATTCAACTGAACATATTCATTAGGTGTATCTCCGTACATG
AAGAATAAAGGAACGTCTCTAGAATATGATACCAAATTTCCGGTAATATTAAACACTATA
GGTGCACTACCTGCCTCAAAACATCTATCTCGGAACCATTGACCATTAAAGTTATGGTCC
GCTCCAAGGGTGTGAATTACTTCAACAGATCGACCTATCATATTACTCATCCAAAACGGA
ATTCCCATTCTCAATCTCTGCGCGGCACCACCCATCCAAGGGTCTCCAGTTTGTACCCTC
GCAGACCATCCTACCCACGGTCCTTCAACTGCCATATAAACCTCCAAGGGGCCGAAGCCC
CTCATTTTATTTTAACAATGATTTAACTAATTTTTTAAGCTCTTCAATCTCTGACTTAAG
TTCTGCAATTTCTGCAGTATGCTCATTGATTGCAGCTGTATTTAAACCAATTACACCGTT
ATAGTTCAAACGAAGTAAAGCTTCACTATCCTGATCGCCTTCAACCAATTCAGGTAAAAT
AGCTTGAACTTCTTGAGCAATTAACCCAGCGTTTGGTTCCCATTTCTGATTACCTTCTTC
ATCTAGGCCTCGCTTCTGCATATAAGTGTAACCATTAATTTTTGAAAGCTTCTCAGAAGC
ATTTTCAAATTTAACTAGGTCCTTTTTAACACGAATATCAGAGCGGACATAAACATCGCG
GACATACGTTGAATAGCTGTCATTCGCCTGAACTAAATGGCCATGAGAAATAATTGCTTT
GCCAACTTCTGTCCAAAAAGCTCCATTTGAATCCCAAAGGCCTGGATGAGATTGATACAT
CGTGCCCCATGATGCTATATGCTGCGCGTTAGCATACATATTAATTCGACCATCGCCGCC
ATGAACTAAACCGGTATCATCATCCCCGATGGCGATAGATGCTCCACCCCATACTGGAAT
TGTGCCGACGCCCAAGTTAACACCAGCACGAAGGCCAGCCGGAGCTTTAAAATCACCATT
AGCCTGAAACACAAATATTGCATTAGGATGGCTGCTATCTCCGCTTTCCGCAGAACCAAC
ACGAATTACTGCATTTCCCCAAGTAGCATTTCCACCCAATCGAATAATACCAAAATCTGC
CTTTGTGGCATATCCTTCGTTAATGATTTGGCTTCTTCCTTTAATTATAGGCATATAACT
ATCTTTCGGAACAGATTCACCAAAGTCAACGAAGAATGGGGCTTCTGTTTGCCATTGATT
ATTATAGCCGTGACCTTGTGTAGCTGTCCATTGTCTTCCATTTAAATATAAACGATAAAA
ATTTAATGAAGCTGCAGTAGCTTGATTTTTGACGTTAAAATCAATATCACCAACACTATT
AAGAGTAATTCCTGCGGTAGTTTTGAAATTATAAAATGAAGCAGTGTCATCTGCTCCTCC
TTTACCGATGTACCATGAATTCGCTCCACCCGATGTTCCAAGTAAATAGCAAGAATCATT
TTCATCGGCAGCAATGTTAATAGCGTCAGAGCCTCTTCTAACGATTAATTTGCCAGTCAT
CGTATCACCGCCTTTACCTACGCGAGTATTCAAGGCAGCATCTAAATTTGTCGCATTGTT
ATATTTCTTCCAGATTGAACCGGCAATATTACCATCTGTATTAAGGAACGCATCACCGAC
ATATACACGCCCTGGAGATTTAAAATAACCTGACGGCATGAAAGTCCATTGTTTTAACCC
ATCTGCTGTTCCATTAGCGGATAAATCACCCTGAGCAATGATCTTATATTCGGATGTACC
TATAGCCATACCAGTGGCAAAGGTGATATTACTTACAGTATTTTTCTGTTTAACAATTGG
CGCAAACTGGTTTTTATTTGAATCGTCAATGGCCTGATATAATGGTGCAATGGTAGTATT
CTGATTCGCGTAAGTATTAGAACCAGAACCGCTAGCAGGGGCTTGAATATATGCTCCTGG
AGCAAGTGAAATTTTCTGATTAGCACCAAGATTAACAGCAGCAGTGATATTCAACAATCC
CCTAAAGTTTTGTTGAGCAGTCCAAGTATGTGATCCGTCTTCAAGAACAACACGGCGAAC
TGTGCAAGTTCTATTTCCAGAATTACCGAACACACGAACGACAAAATAACGGTAGTTTGT
CTGGGATACAGTACCACGCCAAACCTGAACAGTACGTCCTGTGCTGGTGTGTTCGCTAGG
ACCGACAGAAAACATAACCAAGTTTCCGTCAATCGTCCCCCAATCCATATTTGCTGGCAT
ATTCTTTATTTTGTTCAAAGGAACGCTAAACATACTACCAGGGACAAAATCAAAGGTTTG
CCAATCTAACTCCGCTAATTGAGCTTTAATACCACCCATACCCAAAGTAATTGGCAGAGA
ATAAGATGTATATACTTCGTTCCATGCACCCCACGCACCACCAGTATATGCACGCTCAAA
AATACGATCGCGAGTAGTTGCACCCGTTCCTGCGGTAGTGAAACGTTGGAAAATAGCACC
GCCAGAAGCACGCTGTTTAACTTCCAGCAAACCAACAATAACAGTCGGCAAACCTTCACC
TGTAGGACAGTTAATAACGGCATCAGTGCCTAAAATATTATATAACCCGGGAGTTTTAAA
ATCATTCAGATCTCCATCATAGAAAGTCGCTGGTGTGCCATGACCAACCTGCTTCCATTC
CTCCCACTTAGGTGCACTAGCATCCCATGCAGCAGCGAGACAACGAGTATAAACCATACC
CATGCGGGTAGTATAACGCTGAGTTCTCGCGTATAAACCACCTTCAAATATTTCTAAAAA
TCCTTGTGCGGCTGATCCTTCTTCTGGATAATGGCGATCAAATGATGCGATTGCGCTTGA
ACTGTTTCGCCATAAGCCACAATGTTCTAATTCTCCCAAGCTATCAAGGTCAATCGTTTG
TGAAAGTGGACGAGTTGACGCTTGAACATTACGCCACACACCCCATGGACCATCGACGCC
ATTCCATTTAGCAGATAAGGAACGAATATAGACGTTACCAGATCTTACAGTATAACGCTG
AGTGCCAGCAAATTGGCCGGCCACAAATACTTCTAGAACACCTACAGCATTTTCTTCTGG
AAATTTATTTGCTGGTTGCGCATTTGTTGAAGTAGATTTCGACCAAACACCAAGATATTC
TTCAATGGGCCCATACGTATCTAAATTAGCATCAGTAGGTAATTCGCCATTATTTTTTAT
AAATGTTGTGCTATTGACATTTATGTCAGATGGAGTTAAGGTAATGTCCTTTGACCCATC
AAAAGATATACCATTAATTTTCCTAGGTGTTTGCAATTTAGTAGCTGTGCTAGAATTTCC
AATTAAAGTTCCAGTTATATTTTCTGTAACCTCTAAAGACCCATTTATAGTTCCTCCATA
TTTTAAACCTAATTCTATAACACTACCTGAGTCATCTTTTGTGAAAATTGTTTTATCTTT
TAAGTTTATAGCCAATTCACCTTCGGCTAATACTGAAGCAGCAGGACGTTGACCTGCAGT
TTTGCTTCTTTTAAATTGTATTTGTTTTAAAGTAGCCATAAGTCCTCTTAATAATAGCCG
AAATCTTGAACAGAATCCTTAATTACGATTTGGTCAAATCGTGGAACGTGTGAAGGTTGA
GATGCAGGATTCTGTGAAAAAAGGTTTGGCGCAGTTAAATTTCCTGTCATAGTGTCTCCA
GAGCGTAATACCCTAGAGTTTGCATTTGCTGTAACAGTATTTATTGCTCCATCAACATAA
TCTTTGCGAGTAACATCAGATGCTGCGGAAGGAGTAGATGTAACAGTTACTGATGGAGCC
GTTACAGCCCTGGTAAATGTTGTCAAACCTTCTGGCGTAATAGTAATTTGTCCAGTAGTG
ATTTCACTACCTGCAGGTCTAAAACTAATACCACCGGAAGAGCCGCCAACTACAAGAGTA
TTACCAGTGCCGCCACCAGAACGAATTCGGATAGGGGTTTCATTATTACTTAATTGAAGA
TAACTATTTACCCCGTAATTAATAGTAATTGGACCGGCGTAAGTACCACCATTAGCTTTA
GAAACGAAATCGTTATCAGCTGCCTGCGGTTTATTATATTCTGAATATATTTTAAATGAT
TTGTAGAGTACATCGTCACCGGCTGAATTCAATGGAAAATTTCCTTGATGCCAAATGACA
GATCCACCCACAGTTGAACCTACTTTTAAATCAGCCATTATATGCCCCTTTATTTTAATA
TTATTTATAAAGAAAAAGGGAACCCGAAGGCTCCCTCAGTTTAAACTTCTCTAAATTCCT
GTCCAAACACTTTGCCAGTTTTAGAAGAGGCTTGTGTAGGAAGTACCATTATATCTGGAG
GTGAAGCCGATTCACAGACATAATTAACGCGAATACCGTTGACGCCAAATTCGGCAGGTT
TTGAAATGCCGCCATTTCTTGATACTTCAGAAAAACTTAAATTTCTCATGCCGCCTTGAC
CTGCTTGAGCAGTTCTTTGTGCATATATCGTAAATCCTACTGCATTTTCAGGAACAACTA
CATAATCTTCTTTTAATTCCCAAGACCCAGCTTGCCCAGTAAACTCAGCTTGGGTAGAGG
AAATATATCCATTTGATGCATCATAAAAACGAATGGATATGTTTGTAGTTCCAAGATCAA
GTAAATCAGCATCAGCATATAACTGTGCTTTAAGATAAAGAACATCACCAGGAATTAAAT
TATAGTCAGAAAGTTTACTTATAGCAGCTGAAGTTGGCAATCGCGCAATTTCATTATTAG
TTCCACCAACCGCAGACATAAATTGCTCAACGCTTTCGTATGTTCTTTTAGGAAATCCTG
TAGCTCCGACGTCTTCTAAACTATCAAATACAACATCTAACATAGTTTGATAGTCATCTG
TGCTTTTTCTATTACTTAGTTTAACATGCTCTAATGCGATAGCTCTTTTAGAAGAAGTGT
AAAAAGCAGCATATGATACGTCAAATCTTGACAATACTGAATCTGATGGAAAAACTGAAG
TTCCTGCTCCCCTTAACCAAGATACCACTTCAGGAGGAAAATTAACCTTTCCGCTAGTTA
ATATAGCAACAAGTCTATTGTTTGACAAAGAATTCATGAAACTGACAAAAGCGGCAGATG
TTGTATTGTTTGAAGCAGAAAAAGCATATGACTTGCTATCAACTAATGCTCCCGTAGAAG
GGTCAAAAACTCTTAAATGAAGACCTGCACTAAATGTTTGATTTCCAACGGGATTATCCT
GAAATTTAACATATGGTCCCGCAGTAGAAAGCGGGCAAGAACCCGCTATGCTTATTTTAT
ATCTTACTGAATTGCTTTCCGATAAAAATGGCGTTTGGACATATCCTTGTCCAAACTCTG
CCATAAATTTTTCCATAATACCTCTTATTCAATCCACTCAAATTTAACAGTTTTATTCAC
TGGGTCAGGAATAATGCGAACATTACCAATTCGCAAGAAATCGCGAATAGTAAGATTACC
TATTATTCCATTATCAGATGGGATCGCTCCTATATCTGAAGGTTGAGGAGGGTTACCTCC
GTCAAATACCTGAACAAAACTTGACCAAGAGTTTTTGGTTTTCTGCCATGTACGTGTCCA
GCGAGTGGTACGTGCTTCTGGGGTCGTTGGATAAGTAATCCAATCTTGGTAAAGTGAATC
AAGTGTGTTACCAAACTGAGTCAATGTACCAGGAGATTTAACTTCTTCACCACGTTCTAA
GTATGGAAGTCCAGTCACTTCATTAGTTTTTTCAACCATCTTAAAATAACCCGGGAACTG
GTTATAAGTGGCTGAATCATTAATATCGATTGACCAGAATCCTACAGTATCAGATGTTGG
AGCACGGGTATATAAATCAGAGGTTTTAGTACCCTGAGAACGAATTCTCGAGTTAACAGT
TAAACCACCTGAACTTATAGTAGCACCTTTGGCAATGATTAAGCTTTCACCAATCGTTAC
TTGACCAGATGCATTATTAATAGTTAAAGGACGTAATCCATTAAATCCACCGGTCTGATC
GCCAGATGCAGTAAGCATAAAATAGGTGTTAGCACCATCATTACGAATAAAGAATCCATA
ATCACCGTTTATTGCTCTAAAAGCATTCGATGATTTACTGATAAATTCGCCATTAGCAGT
AACTGAACTACCGAATGTTGCAACGCCATTCGCATTCAATGTTCCTGAAGCATTCACATT
TATCGGCGTTACTGTACCGTTGATATTAAATGTTATATTTCCAGCTTTATTACGCTGAGA
ATAAAAATGACTAGACGTTTCATCACTAACTTCAAATACGGTAGAACGTGTTGTGTCTGA
TTCCCCACTAAATTGATTTCCCCACACTCTGACTGTCATCGTTTGAGCTGGGTTTGTTCC
AGTTTGAGGTCCTTTCTCAAAAATCAGACGAGTTGCCGTTCCAGTATTAGAAATAGTTAA
TGTACTATTTGCTGAAACTGATCCACCGAATGTAGCAGTACTAGATGATACAAGAGGGGC
ACTCAGATTCGTTTGTTGGGTTAAGGTTAGTGAACCATTAACTGTTTGTGCAATGTCCCT
ACGAATGAACTGGAGCGAATCTAGACCATCTAATAAATTACTGTCTACAGCTTTTGCTTT
TAATGGCAAATAATTTGCTAATACGCGATTTAATTCATATGGTGATACTGCATAACCATT
TTTCTCATATGATTCTAATGGCTGTGTTGAACCTACCGTATCATTACCAACGAATGTAAT
TGAACCAGATGAAGTTTTAACAAAACCTCTTATTGCAGTAGTAGCTGCCCAAGTAGGTTC
ACTCTGCACAATCCATTTTAAATTTTTTGGAGATACAGCAGTATTTGCTGACGTTCCAGC
CACAGTTTCAGACTGAGTTGCAACTTTAATGACACCTTCCTGTGCTTCAGTAGATTTAGT
ACCTAAAAGCTTTTTAGGAGTTATTAAAACATTATCTAATGTTCCTGCAGCAGCTTCAAC
TTGTGTAGCTACACGAAGTGTACCACGCTGTGTCTCATTTGCTTCAAGAATATTAAGGGT
ATAATGGTCCCAGAGAGTTCCTGATTCAACTAATCCAGATAGAGCAACAACAGAAGTACG
ATCAGTACTATTAAATCTGGTTTTAATTTTTAATGGTGTAGAGATACGAGTATCGTCGAC
GCCTGCGTCGAATTCAACCTGTGTAGCAATTTCAGCTATACCACTTAAACTTTCAGTTGC
TTTACGGTCATTTAAAGTTTTAGGAGTGACTGCGCGAGTATAATCAGTTCCTGTATTAAC
TTCACTTTGCGTAGCAATTTCAATTAAACCAATTCTTGCATCAGTTGATGTCTTTTTATG
TAACGTTTCTGGCGTTACAACCGCATTTGCCCATCCTTCTTGGCTTTGTCCAGCAATTAC
TTCACTTTCAACTGCTAAAATTACTGCGCCTTGTTGCGTTGGAGTAGCTTTATACTGATC
CAAAGCTTTAGGTGAAACAACTAAATTATTAGTGTTTTTATTATAAACATTCGTACCATT
TAATTCACGACTAGAAGCTGGAGTAGCTCCTGCGGTAGATACAAAAGTTACAATACCAGA
TAATGATTCAGAACCTTGACGAGCTTGAAGCTTTTTAGGAGTGATGATTGTAGTATCATC
AGTACCTGTATTAGTTTCCTGCTGCGTAGCAATTTCAGCGACACCTCTACGAGTTTCTGT
AGCAGTTCTTTCATTCAGCTTTTTAGGAGTGATGATAAGGTCATCTGCAAAAGAGAATGT
GGTGTTCTGATTTACTTGAGCAGTAGTTGCTATTCTTGCAATACCTCTGCGAGTTTCAGT
AGCAGTACGATTAGCTAACGTTTCTGGAGTAATTGCCAATTCTTTTTGCGGAGAATTTTC
TAAATCAGCATTTGCTTGAGCTTGTGTAGCTAAAGCAATTACGCCTAATCTTGCTCTAGT
AGAATTATTTAAAGAATCGACTCTTTCAACTGTTGGAACGTTTTGCTGTACAACCCAGTA
TTTTCCATCAGAATCTTCTATATAAGCAAGTTGCAAAACTGGTACATAATTAGTTTCACC
ATTAAAAACTAATTCTTGAACTGTTACCCATTCAGCTTCAGGCGGATATTCTGAACGTTT
TGGGAATTGCAGCAATTGAACTGAAGAAGCAATTTTATCTTCACCGGCAGCTTTGATTTT
AACTGTTTGTCCTTTTCTCATGTAATTCATGGAAATTTTAACAGTATCACCAACAGAAAT
ATCAGTCGGAAGCTGAAGCTCAATTGTTTGAGTTGTTCCATTATTCGCACCAAATACCAT
AACTTCTTCATTTGGACGAATGTTTGAATTAGTCGTTATAATGCGTAAACGTGCTTTACT
ATCCCCGTCAAACAATCTCCACAATTTCTCATTATCATCAAACATCAAGAAACCGTCAAT
CGATGTACGGCCTTCAATGGAATGAGTTCCAACTTCTTGCACTGACGTTGTTTCATCATA
TGTCGTAACAATTGTATGATAAAGTGGATTTAATTTATCTAAATCAACGAAATTAATGAT
ATCTCCATGATTGGCAAATCTTGGAAGTTTAACATTAATCGGTGCGGCAGAAGTAAATCT
ACGTACGATAAAATCATTAGATTGTGCTTGATATGCAGTCGATGGAGTAACAATTGCAGC
TTCTCTGCTATAATCAGCAATATACATTTGCCACAAACGGTTATTAAAAATGAATACCAT
CTGTGACTTTGGATGAGTCATTAAAACTGAACGTACCTGTTCACCTCTAAAATTGACAAT
ACTCCGATTTGAAGAATTTATTTTAACCTGATTTATGCCAGGTTTTCCACCGATATCTTG
AATTATTACGGTTTCTCCATCAAGCGGAGAAGGTGGTAAAGTAAACTCAATGTCATTGCC
AACTGATGTATCTACTGAAATTGCTTCTCCGGATTTTAATTGATATGGTCCAGATGATAC
GGTTGTATATACGGCATCAGTACGTAATGCTTTCCAACGAATTCTATTAAAATTTCCAGC
AGGTTTTGGAATATTATCCGTTGCTGCCCAAAAACGATTATTATAAATGATTACAAAATC
TTTTAAATATCCACGAGTTGGATCATATTGCTGAACTGTGTTTTCTTGAATTAAGTAATC
AACGTTAACACCGTCAGTTCCTACGGTACGGTCAGCTAAAGCTACGTTGATTATTTTATC
GCCACCTGCGTCCAGACCATCTTCTGCTCTGAACTTTCTTTTAATCTCGGCCATTCTCCC
GGGCTCCTATTGTGTTTTCAATAATAAGTATTTATACCTGTTTACTTTAAGATTTAGATA
GTATATAATAGAAATCTCACTAATTGAACGAGGTTCATATGGATTTAGAAATGATGCTGG
ATGAAGATTACAAAGAAGGAATTTGCTTTATTGACTTTAGTCAAATTGCTCTTTCAACTG
CTCTGGTAAACTTCCCAGATAAAGAAAAAATTAATTTATCGATGGTTCGTCATTTGATAT
TGAACTCAATTAAGTTTAATGTCAAAAAAGCAAAAACGCTTGGATACACTAAAATTGTAC
TGTGTATTGATAACGCGAAATCTGGATATTGGCGTCGTGATTTCGCGTATTATTACAAGA
AAAACCGTGGAAAAGCACGAGAAGAATCTACTTGGGACTGGGAAGGTTATTTTGAATCCA
GTCATAAAGTTATAGATGAATTGAAAGCTTATATGCCATACATTGTTATGGATATTGATA
AGTATGAAGCAGATGACCACATTGCTGTTCTTGTTAAAAAGTTCTCTTTAGAAGGACATA
AGATTTTAATCATTTCATCGGACGGTGACTTTACTCAACTTCACAAATATCCAAATGTTA
AGCAATGGTCACCGATGCATAAGAAATGGGTTAAAATTAAAAGCGGTTCTGCTGAAATTG
ATTGTATGACTAAAATTCTTAAGGGTGACAAAAAGGATAATGTTGCTTCAGTTAAAGTAC
GATCTGACTTTTGGTTTACCAGAGTTGAAGGTGAACGAACTCCTTCAATGAAAACTTCAA
TCGTTGAAGCTATTGCTAATGACCGTGAGCAAGCTAAGGTGCTTCTCACAGAATCTGAAT
ATAATCGCTATAAAGAAAATTTAGTTCTAATTGATTTTGATTATATTCCTGATAATATTG
CTTCAAACATTGTGAATTACTATAATTCATATAAATTACCACCGCGTGGCAAAATTTATT
CATATTTTGTAAAAGCGGGTCTTTCTAAATTAACTAATAGCATTAATGAATTTTGAGGTG
AATAATGGCTAAAAAAGAAATGGTTGAATTTGATGAAGCTATCCATGGCGAAGACTTGGC
TAAATTTATTAAAGAAGCATCTGACCATAAACTGAAAATTTCCGGTTATAATGAACTGAT
TAAAGATATTCGAATTCGTGCCAAAGATGAACTTGGCGTTGATGGTAAGATGTTTAATCG
TCTATTAGCTTTGTATCATAAAGATAATCGTGATGTGTTTGAAGCTGAAACTGAAGAGGT
AGTTGAACTTTATGACACAGTTTTCTCTAAATGATATTCGTCCGGTCGATGAGACCGGTC
TTTCAGAAAAAGAACTTTCAATCAAGAAAGAAAAGGATGAAATAGCAAAGCTTCTTGATC
GTCAAGAAAACGGATTTATTATTGAAAAAATGATAGAAGAGTTTGGAATGAGTTATCTTG
AAGCTACAACAGCATTCTTGGAAGAAAACTCTATTCCTGAAACTCAATTTGCTAAATTTA
TTCCTTCGGGTATAATTGAAAAAATTCAGTCAGAAGCCATTGACGAAAATCTTTTACGTC
CTTCTGTTGTTCGTTGTGAAAAAACTAATACATTAGATTTTCTACTATGATTAAACTCCG
CATGCCTGCTGGTGGTGAAAGATACATTGATGGTAAATCAGTTTATAAATTATACTTAAT
GATAAAACAACATATGAATGGAAAGTATGATGTTATTAAGTATAATTGGTGCATGCGGGT
GTCTGATGCCGCTTATCAAAAGCGAAGGGATAAGTATTTTTTCCAAAAGCTATCAGAAAA
GTATAAATTAAAAGAATTAACTTTAATTTTTATTAGCAATCTTGTAGCTAACCAAGATGC
TTGGATTGGTGATATCTCTGACGCTGATGCATTAGTATTTTATCGTGAATATATCGGACG
CTTAAAGCAAATTAAATTTAAGTTTGAAGAAGATATTAGGAATATCTATTATTTTAGTAA
AAAAGTTGAAGTTTCTGCTTTTAAAGAAATCTTTGAGTATAATCCAAAAGTTCAGTCGAG
TTATATTTTTAAACTACTTCAATCGAATATAATTTCGTTTGAGACGTTTATCTTGCTTGA
TTCGTTTTTAAATATAATCGATAAACATGATGAACAGACTGATAATTTAGTTTGGAACAA
TTATTCTATAAAGTTAAAAGCTTATAGAAAAATTTTAAATATTGATTCACAGAAAGCTAA
AAATGTTTTCATTGAAACTGTGAAATCTTGCAAGTATTAATTGCTTATTATAAATAGATT
ATAATTATCTCACTGACCAGCTATGAGGTCATACATCGTCATAGCACCAACTGTTAATTA
AATTAAAAAGGAAATAAAAATGTTTAAACGTAAATCTACTGCTGAACTCGCTGCACAAAT
GGCTAAACTGGCTGGAAATAAAGGTGGTTTTTCTTCTGAAGATAAAGGCGAGTGGAAACT
GAAACTCGATAATGCGGGTAACGGTCAAGCAGTAATTCGTTTTCTTCCGTCTAAAAATGA
TGAACAAGCACCATTCGCAATTCTTGTAAATCACGGTTTCAAGAAAAACGGTAAATGGTA
TATCGAAAATTGCTCATCTACCCACGGTGATTACGATTCTTGCCCAGTATGTCAGTACAT
CAGTAAAAATGATCTGTACAACACTGACAATAAAGAGTACAGTCTTGTTAAACGTAAAAC
TTCTTACTGGGCTAACATTCTTGTTGTAAAAGACCCAGCTGCTCCAGAAAACGAAGGTAA
AGTATTTAAATATCGTTTCGGTAAGAAAATCTGGGATAAAATCAATGCAATGATTGCAGT
TGATGTTGAAATGGGTGAAACTCCGGTTGATGTAACTTGCCCGTGGGAAGGTGCTAACTT
TGTACTGAAAGTTAAACAAGTTTCTGGTTTTAGTAACTACGACGAATCTAAATTCCTGAA
TCAATCTGCGATTCCAAACATTGACGATGAATCTTTCCAGAAAGAACTGTTCGAACAAAT
GGTTGACCTTTCTGAAATGACTTCTAAAGATAAATTCAAATCATTTGAAGAACTGAGCAC
TAAGTTCAGTCAAGTTATGGGAACTGCTGCAATGGGTGGTGCCGCTGCAACTGCCGCTAA
GAAAGCTGATAAAGTTGCTGATGATTTGGATGCATTCAATGTTGATGACTTCAAAACAAA
AACTGAAGATGATTTTATGAGCTCAAGCTCTGGCAGTTCATCTAGTGCTGATGACACAGA
CCTGGATGACCTTTTGAATGACCTTTAATAGATTATATTACTAATTAATTGGGGACCCTA
GAGGTCCCCTTTTTTATTTCAAAAATTTTTTCACAAAACGGTTTACATCCTTGTCCTTCC
ATGGTACTATACAACTATCGGCAATACTGCTGATGATTAAAGAGGAAAATAATATGGCTA
AAGTTGATATTGACATCGTTGATTTTGAATATATTGAAGAAATTATTCGTAATCGTTATC
CTGAACTTAGTATCACAAGTATTCACGATGATCCCAATTATTGCAATTTTTCTATTGTCA
TTGAAGGTCCTCTTGAAGACCTCACCCGCTTTATGGCTAATGAATATTGTGATGGTATGG
ATTCTGAAGACGCAGAATTTTACATGGGATTGATTAAACAATAATTATCAAGGGGCTATT
ACAAGCCCCGTTAAAATGAGGAAAACATAATGTATATCGGTAAAAAGTATGAACTTGTTC
CAAGACTTATTGATACATTTATTAATTATCGCCCACGTTCTAATTCAGCAATAGTTAAAA
TTATTCAAGAAAATGGTGGATGGTTTGAAGTTAAAGAAGCTTTCTTTGTTGATGGATTTA
GAGTAATAAAACACATTGAATGCGCAAATGGAAAGCATTTTTACTTTAACGTTTGTGAAG
ACGAATTTCATTGTTTTCGTGAGTATAAAGAACCGACTTCTGAAGAAGATGGAGCCGAAG
ACATAGTTTCTGGCGTAACAAAAATTCACTGCATTGTTGACGAAAACAATGTAGATGAAA
TCATTGAACTTTTGCGAAAAACTTTCAAAAAGTAGTTTACAAAAGGGTAGTAGTGTGATA
CTATTACCCTATCAAAACTAATGGAGAAAAGAAAATGTTCGCACCTTATATTATGGCAGC
AGTTATGTTGGTCTGTTTATATCTTTTGATTAAAGCTTGCTAAGGAGAATAAAATGAGAT
TACAACGTCAGAGTATCAAAGATTCAGAAGTTAGAGGTAAATGGTATTTTAATATCATCG
GTAAAGATTCTGAACTTGTTGAAAAAGCTGAACACCTTTTACGTGGCATGGGATGGGAAG
ATGAATGTGATGGGTGTCCGCTTTATGAAGACGGTGAAAGCGCAGGATTTTGGATTTATC
ACTCTGACGTCGATCAGTTTAAAGCTGATTGGAAAATTGTGAAAAAGTCTATTTGAGGAA
AATATTATGTCTGACATTCATGTAATTGGAATTTCACAAGTAAATATTCGCTGCCAATTT
AAAACTGTACCCGGGGTGACTCATATTACTTTATCACACGACCCATATTCTCGTGGAAGA
CAGTTAACTGGTGTAATTAAGTTTTTCGGTGGAATTGGTGGTAATGAATTTACTGTAGGC
GATGAAGAAATTGTTGGCTGTAAATTAAAAGTTCAGAAGGGCGTGTTAGAACTTTTTAGT
GATGAAGTTTTTGATGAAATCTCACGAGCAGTTAACAAAGGAATGTTAACGTTAATTAAA
ATGATTAAAGCTAGCGGATATGTTCCTGATCCTTTTTAATAGGAGATAATATGATTTTTG
TATTTGAATTTATGAATGATGAATTTGATTATGCAATTTTTAACGCATTGCATAATCCTG
ATTTAAGTGAATTTAATGAAATGTTTTCTGATGCTTTGAGTATGTCAGAAGAATACTGTG
GAGAATGTCAACGTGTTTGTGTGACAGTTTTTGAAAACAAAGAAAAGACGTATGAAGAAT
TATTCTTTGATGCTAATAAAGCCACTGAATGGTTTATCGAAAGAGGATTTGCGTAATGAT
TAAATTGGTATTCGCTTATTCTCCAACTAAAACAGTTGAAGGCTTTGACGAATTAGCATT
TGGTTTAGGTGATGGTTTACCATGGGGACGAGTTAAAAAGGACCTCCAGAATTTTAAAGC
TCGTACCGAAGGTACAATTATGATTATGGGTGCTAAAACGTTCCAGTCATTATCTACATT
ACTTCCTGGTCGTAGTCATATTGTAGTGCGTGACCTTGCGCGTGATTATCCTGTAACTAA
AGACGGCGATTTAGCACATTTCTATATTACTTGGGAGCAATATATAACTTACATTTCTGG
CGGCGAAATTCAAGTTTCAAGTCCTAATGCACCATTCGAGACTATGCTTGATCAGAATTC
CAAAGTAAGTGTAATTGGCGGGCCTGCTCTGTTATATGCTGCGTTACCTTATGCGGATGA
AGTAGTTGTTTCCCGCATTGTTAAAAGGCATCGTGTTAATTCAACAGTTCAATTAGATGC
AACTTTTCTTGATGATATAAGCAAGCGTGAAATGATTGAAACGCATTGGTATAAAATAGA
TGAAGTAACAACCCTTACGGAATCAGTATATAAATGAAATAACGCGTGGCGGAAATATGA
ATTTTAATTATTACCCTATTCTATTAGAAAAAGACACGAAACAGCCAAAGTGGCAGGGTC
CTCAGTTTATTAAAGGTGTCTATCAATTAGTAGTTCCTAAAGACAAGATTTATAGCAGTT
GTTTCACTGAATCCGCTTGCAGTATTTTCGGTAATAGTTCACCGTATTGGAATTTTGACA
TAAAGCTGGATAGAAATATCGATATTTGGTTGAAAGCTATGGATATTGGTAATATCACGT
TTGATGAGAATAATTATCATATTATTGGTCGCTTTTCCAAACGCGGTAAAGAATTATATT
TCACGCCTGAAATTGAGAGAAAATTTGATGCTAAACCGTATTGATTATTTAGCTTCATAA
AATGTATTATAATTTATATAAGGTACTAAATGAAACAATACCAAGATTTAATTAAAGACA
TTTTTGAAAATGGCTATGAAACCGATGACCGAACAGGCACAGGAACAATTGCTTTGTTCG
GTACTAAATTACGTTGGGATTTAACTAAAGGTTTTCCTGCGGTAACAACTAAGAAGCTTG
CGTGGAATGCATGCATTGCTGAGCTTCTATGGTTTATTTCCGGTTCTACCAATGTTAATG
ATTTACGTCTTCGCACACATGGTAGTCTTATTCAAGGTAAAACTATTTGGGACGATAACT
ATGAAAACCAGGCTAAAGACCTAGGATATCATAGTGGTGAACTTGGTCCTATATACGGTA
AACAATGGCGTGATTTCGGCGGTGTGGATCAATTAGTTGAAACTATTGATCGTATTAAAA
AATTACCAAATGATAGGCGTCAAATTGTTTCAGCATGGAATCCAGCTGAACTCAAGCAGA
TGGCATTACCGCCTTGTCATATGTTCTATCAGTTTAATGTGCGTAATGGCTATTTGGATT
TGCAGTGGTATCAACGATCAGTAGATGTTTTCTTGGGTTAATTGAGGCCTGAGTATAAGG
TGACTTATACTTGTAATCTATCTAAACGGGGAACCTCTCTAGTAGACAATCCCGTGCTAA
ATTGTAGGACTGCCCTTTAATAAATACTTCTATATTTAAAGAGGTATTTATGAAAAGCGG
AATTTATCAGATTAAAAATACTTTAAACAATAAAGTATATGTAGGAAGTGCTAAAGATTT
TGAAAAGAGATGGAAGAGGCATTTTAAAGATTTAGAAAAAGGATGCCATTCTTCTATAAA
GCTTCAGAGGTCTTTTAACAAACATGGTAATGTGTTTGAATGTTCTATTTTGGAAGAAAT
TCCATATGAGAAAGATTTGATTATTGAACGAGAAAATTTTTGGATTAAAGAGCTTAATTC
TAAAATTAATGGATACAATATTGCTGACGCAACGTTTGGTGATACATGTTCTACGCATCC
ATTAAAAGAAGAAATTATTAAGAAACGTTCTGAAACTGTTAAAGCTAAGATGCTTAAACT
TGGACCTGATGGTCGGAAAGCTCTTTACAGTAAACCCGGAAGTAAAAACGGGCGTTGGAA
TCCAGAAACCCATAAGTTTTGTAAGTGCGGTGTTCGCATACAAACTTCTGCTTATACTTG
TAGTAAATGCAGAAATCGTTCAGGTGAAAATAATTCATTCTTTAATCATAAGCATTCAGA
CATAACTAAATCTAAAATATCAGAAAAGATGAAAGGTAAAAAGCCTAGTAATATTAAAAA
GATTTCATGTGATGGGGTTATTTTTGATTGTGCAGCAGATGCAGCTAGACATTTTAAAAT
TTCGTCTGGATTAGTTACTTATCGTGTAAAATCTGATAAATGGAATTGGTTCTACATAAA
TGCCTAACGACTATCCCTTTGGGGAGTAGGGTCAAGTGACTCGAAACGATAGACAACTTG
CTTTAACAAGTTGGAGATATAGTCTGATCTGCATGGTGACATGCAGCTGGATATAATTCC
GGGGTAAGATTAACGACCTTATCTGAACATAATGCTTCCATTTAATATTGCATCATATGC
TACGTTAGTTCATATTGTAGCTAAGATGTGTAATCTTATTCCGGGAGATTTGATATTTTC
CGGTGGTAATACTCATATCTATATGAATCACGTAGAACAATGTAAAGAAATTTTGCGTCG
TGAACCTAAAGAGCTTTGTGAACTGGTAATAGGCGGATTGCCTTATAAATTCCGCTATCT
TTCTACTAAAGAACAATTGGAATACATTCTTAAACTCAGGCCTAAAGATTTCGTTCTCAA
AGATTATCAGTCCCACGGCGTCTTGAAAGGAAAAATGGCGGTGTAATTTTAATTTAATTG
TGAGGATTTATGATTTTACGATTTAAAGATACTTCTGACGTTGTTCTTTTTACACTTCCT
AATCCAAGTGAGCTAGAAGTTCCAGGACCAAATCAGCCTATTATCATTTATGGCAAAAAA
TATTATACTCATAAAATGACTCGTGAGTATTTTGATAATAAAATTTCCACAGTTAAAACT
TCTTCTGACTGTTACTACGATATTACTGTTTTAACGGAAAAACAATATGCCGAATTATCA
CCGCGCGGGCCGTCTATGTCGGGTAGTGAATAAATACAAATCCGACTTTGATGTTAATAT
TCACCGTGGTACATTTTGGGGAAATTACGTCGGTAAAGATGCTGGCAGCCGGGAGGCTGC
CATTGAATTATTCAAAAAAGATTTTATACGTCGAATTAAATCCGGAGAAATAACTAAAGC
ACATTTAGAGCCTTTACGTGGAATGAGGCTAGGATGCACATGTAAACCAAAGCCGTGTCA
TGGTGATATAATAGCTCATATAGTTAACCGATTGTTTAAAGACGATTTTCAAGTTGAGGA
CTTATGCAATTAATTAATGTTATCAAAAGTAGTGGTGTTTCTCAGAGCTTTGACCCGCAA
AAAATTATTAAAGTTTTATCTTGGGCAGCTGAGGGAACATCAGTAGATCCTTATGAATTA
TATGAAAATATTAAATCTTATCTCCGTGATGGAATGACAACTGATGATATTCAGACTATT
GTCATTAAGGCCGCTGCGAATTCTATTTCGGTTGAAGAGCCTGATTATCAATATGTAGCT
GCACGTTGTCTAATGTTCGCTCTTCGTAAGCATGTTTATGGGCAGTATGAACCACGTTCA
TTTATTGACCATATTTCTTACTGTGTAAATGAAGGTAAATATGACCCCGAATTATTGTCA
AAATATTCAGCAGAAGAAATTACATTTTTAGAATCAAAAATTAAGCACGAACGGGATATG
GAATTTACTTATTCCGGGGCGATGCAATTAAAAGAAAAATATCTAGTTAAAGATAAAACC
ACTGGTCAAATTTATGAAACTCCACAGTTTGCATTTATGACTATTGGAATGGCATTGCAT
CAAGATGAACCTGTTGATAGATTAAAACATGTTATCCGTTTTTATGAAGCGGTATCTACT
CGACAGATTTCATTACCAACTCCTATTATGGCTGGTTGCCGTACTCCAACTCGGCAGTTT
AGTTCATGCGTTGTTATTGAGGCAGGTGATTCATTAAAGTCTATCAATAAGGCTTCCGCT
TCAATTGTTGAATATATCTCTAAACGCGCTGGAATTGGTATTAACGTTGGTATGATTCGT
GCTGAAGGTTCTAAGATTGGCATGGGTGAAGTACGCCATACTGGTGTTATTCCTTTTTGG
AAACATTTTCAGACTGCTGTTAAATCATGCTCACAGGGTGGAATTCGTGGCGGCGCTGCT
ACTGCTTATTATCCTATTTGGCATTTGGAAGTTGAAAATCTTCTTGTTTTGAAAAATAAC
AAAGGCGTAGAAGAAAACCGCATTCGTCATATGGATTATGGTGTTCAACTGAATGATTTG
ATGATGGAACGTTTTGGAAAGAACGATTACATTACTTTGTTCAGTCCCCATGAAATGGGC
GGAGAGCTTTATTATTCTTATTTTAAAGACCAAGACCGTTTCCGTGAATTATACGAAGCA
GCAGAAAAAGACCCTAATATTCGTAAAAAGCGTATTAAAGCCCGTGAACTATTTGAATTG
CTCATGACTGAACGCTCAGGAACAGCAAGGATTTATGTACAGTTCATTGATAATACGAAT
AACTATACTCCGTTTATTCGTGAAAAGGCACCTATTCGTCAGAGTAACTTGTGCTGTGAA
ATTGCTATTCCAACAAATGATGTGAATAGTCCTGATGCTGAAATTGGATTGTGTACTCTC
TCTGCATTCGTACTGGATAATTTTGACTGGCAAGACCAAGATAAAATTAATGAATTGGCA
GAAGTCCAGGTTCGTGCTCTTGATAATCTTTTGGATTACCAAGGATATCCAGTTCCTGAA
GCAGAAAAAGCTAAAAAGCGTCGTAACCTCGGTGTAGGTGTTACCAACTATGCAGCTTGG
CTGGCAAGTAACTTTGCTTCTTATGAAGATGCTAACGATTTAACACATGAACTATTTGAG
AGATTACAGTATGGACTCATTAAAGCATCCATTAAGCTCGCCAAAGAAAAAGGACCTTGC
GAATATTATTCAGACACTCGTTGGTCTCGAGGCGAATTACCTATCGACTGGTACAATAAA
AAGATTGACCAAATCGCAGCTCCAAAATACGTTTGTGACTGGTCGTCGCTGCGGGAAGAC
CTTAAGCTCTTTGGCATCCGTAATAGCACATTATCAGCACTTATGCCATGTGAGTCATCT
TCCCAAGTTTCTAACAGTACAAACGGTATCGAGCCTCCACGTGGACCAGTCTCTGTTAAA
GAATCAAAAGAGGGTTCCTTTAATCAAGTCGTGCCCAATATTGAACATAACATAGACCTA
TATGATTATACATGGAAATTAGCTAAGAAAGGTAATAAACCTTATCTTACGCAGGTAGCT
ATTATGCTGAAATGGGTATGTCAATCAGCTTCAGCGAATACATATTATGACCCGCAGATT
TTTCCAAAAGGAAAGGTTCCAATGTCAATAATGATTGATGACCTTTTGTATTTTTGGTAT
TTTGGCGGAAAAAATTTCTATTATCATAATACCCGTGATGGTTCTGGTACTGATGATTAT
GAAATAGAAACTCCAAAAGCTGAAGATTGTTCATCCTGTAAATTATGATATAATTTGACT
CACGGACGAGTCACCAACTATTAACTAAGTGGAAAATTTATGAGCACAGTTTTTAATACA
AATCCAGTTGATGTTTTAAAAGAACCTATGTTTTTTGGTTCAGGTCTTGGTATTGCGCGT
TATGATATTCAACGTCATAAAGTTTTTGAAGATTTAACCGAAAAGCAGTTGTCATTTTTC
TGGCGTCCTGAAGAAGTAAACTTAATGATGGATGCTGCACAGTTTAATAAGCTTCCTCAA
TATCAGCAGAATATTTTTACTAATAATCTGAAGTATCAATCACTTCTAGATAGCATTCAG
GGTCGTGCACCGTCTGCTGTACTTATGTCATTGATTTCAGACCCAAGCCTTGATACATGG
GTTGCTACATGGACTTTTAGTGAAACCATTCACAGTCGTTCATACACTCATATTATGCGA
AATCTTTATACTGATCCGTCAAAGGTATTTGATGAGATTGTATTAGATAAAGCTATCATG
AAACGTGCTGAATCTATTGGTCGTTATTATGATGATGTTCTGGACAAGACACGTATTTGG
GAAAACGCCAAAGAAAACTTTAGTTATTTTGCTGAAAATGAATATGATTTAGAAGCATAT
TCAAACAGTCTCACTCATTTGGAAGAATGTAAACGAGACCTGATGAAGTCGCTTTATCTC
TGTCTGCACGTTATCAATGCTCTGGAAGCTATTCGTTTTTATGTATCCTTTGCATGTACT
TTCAACTTCCATAAGAACATGGAAATCATGGAAGGTAATGCGAAGATTATGAAATTTATT
GCACGTGATGAGCAGCTTCATCTTAAAGGTACACAATATATTATTCGTCAACTTCAATCC
GGTACTGATGGTGATGAATGGGTTAAAATTGCCAAAGAGTGTGAACAAGAAGCAGTTGAT
ATTTTCATGGAAGTTAACCGCCAAGAAAAAGATTGGGCAGTTCATTTATTTAAAGATGGC
GATGTTCCTGGATTAAATACAAATAGCATGTGGAGCTTTATTGATTACTTAACTGTATCT
CGTATGAAACAGTGTGGTCTTCCATGCCCGATTACCGGTGCTCCGGTTAAACATCCATAT
CCTTGGATTCGTGAATACCTTAATTCTGATAATGTTCAATCCGCGCCGCAAGAAGTAGAA
CTGTCATCTTATCTTGTTGCACAGATTGATAATGATGTTGATGATAAAGTTATGATGAGT
TTTAAAAAATATTTTTAAGGAGTGGGCCGCAAGGCCCATTTTATTATGAAAGAAATTGCA
ACAGAATATTCATTTATTAAATATACTGAGCTAGAATTAGACGACAACGGAAGTATAAAA
CAATTATCTATTCCAAACAAGTATAACGTAATTTATGCTATTGCTATAAATGATGAGCTT
GTTTATATTGGAAAAACTAAAAATTTACGTAAAAGAATAAATTATTATAGAACTGCTATT
AATCGTAAAGACAAAACGTCTGATTCTACTAAATCTGCATTAATTCATGCTGCGCTAAAG
GAAGGAAGCAAAGTTGAATTTTACGCCCGCCAATGTTTTAATCTTTCTATGACAAATGAG
TTAGGTACAATGACAATCGCAACGATTGACCTAGAGGAGCCGCTATTCATTAAACTGTTT
AACCCGCCTTGGAATATTCAACACAAGAAAAATGATGCTTCCACATGGAGTGTGGTACTA
TATTCAAAACACAAAAGAGGATACACAATGCAAGAACTTTTTAACAATTTAATGGAACTA
TGTAAGGATTCACAGCGTAAGTTTTTTTACTCGGATGATGTAAGTGCATCTGGAAGAACT
TACAGAATTTTCTCATATAATTATGCATCTTATTCTGATTGGTTACTTCCAGACGCATTG
GAATGTCGTGGAATCATGTTTGAAATGGATGGAGAAAAACCAGTAAGAATTGCTTCTCGT
CCTATGGAAAAGTTTTTTAACTTAAATGAAAATCCATTCACGATGAATATCGATTTAAAT
GATGTTGATTACATTCTGACAAAGGAAGATGGGTCTTTGGTATCAACTTATTTAGATGGT
GATGAAATTCTGTTCAAATCAAAGGGTTCAATCAAATCCGAACAGGCTTTAATGGCTAAT
GGTATTTTGATGAATATTAATCACCATCGGTTGCGTGATAGACTTAAAGAATTAGCTGAA
GATGGATTTACTGCTAACTTCGAATTCGTCGCTCCAACTAATAGAATCGTTCTTGCTTAT
CAAGAGATGAAAATCATTTTATTGAATATTCGTGAAAATGAAACAGGCGAATACATTTCA
TATGATGATATTTATAAAGATGCTGCTCTTCGTCCATATCTAGTTGAACGATACGAAATC
GATAGCCCTAAATGGGTAGAAGAAGCTAAAAATGCGGAAAACATCGAAGGCTATGTTGCT
GTGATGAAAGATGGTTCTCATTTTAAAATTAAGTCTGACTGGTATGTGTCTCTTCATAGC
ACAAAAAGTTCATTAGATAATCCAGAAAAATTGTTTAAGACTATTATTGATGGTGCATCA
GATGATCTTAAAGCAATGTATGCTGACGATGAATATTCATACAGAAAAATTGAAGCATTT
GAAACGACTTATCTGAAGTACTTAGACCGAGCTCTGTTTTTAGTTCTTGACTGTCATAAT
AAGCATTGTGGTAAGGATAGAAAGACTTATGCGATGGAAGCGCAAGGTGTTGCTAAAGGT
GCTGGAATGGATCACCTGTTCGGTATCATTATGAGCCTATATCAGGGGTACGATAGTCAA
GAAAAGGTCATGTGCGAAATCGAACAGAATTTTTTGAAAAATTATAAAAAATTTATCCCA
GAAGGATACTAAGCTGTTTACAAGTTCCTCATGTTGTGTTATAGTAGTCTTACTGACATA
ACATGAGGACTTTATGATGGATTTGCAGCTTATTACTACTGAAATGGTCGTTGAAGCATA
CGGTGATACTACAGATGGGATTTCTGTATTCAAAGGAAATCGTCGAGTTGGATATATCAC
CGATCTTAAGAAAGATTTAGCTAAGCAAGTCAAGCGGAAAACGACCATTAAAGAATATCG
AAATCGTCGTCTTGAGCAAGCCCGTGATATGCTTCCTGATGCGGTTGAGGAGATGAAAGT
CTTTTTAGAAAATCAACTCGCTAAGTATGATTGTGAAGTGTTTATTAATCAGACTCAACC
TAATGTTCATATCAATAGCTGCAAATGCTATATCATCGTTAATCCTTTGACAGGAAAGCA
TCGTCTTGGAATTAGTAATCCAAATCGTAGCGCATCGGATATGGCAGAAGATGTTGCGGC
GTGCTTTAAAATTTCTAAATCTCCGGCTGAACATCATATTTTAATTAACGGGCTTGCTCA
AGACGATATTATAGAGGTTATTAAAACTTTATGCATGTAAGTAATTTTACAGCTGGATTG
CTATTACTTGTAATAGCATTTGGCGGAACATCTATTATTTTAAAAAATAAGGTAGAAAGA
TTAGAAACGTCTGTTGTAGAAATTACAAAAACGGCCAATGAAAACGCCTTAGCATTAAAT
AATTTGCGAATTCAGTATAATTATATTGATGCGATGAATAATAAAAATCGTGAGGCGATT
GCTGCTATTGAGCGTGAAAATGAAAAACTGCGCAAAGATGCAAAGAAGGCGGATGTGGTG
GCTCGTAAGCCAGGATTGGTTGAAAAACAAATCAACAACTCCTTCAACAAGTTCGCAGAA
GACATCCAGGACCTTTCTAAATGATTAAATTATCTGCAGTAATATTATCTATTGGTCTTC
TAGTTGGTTGTTCGACAAAGCCTCTAGAAGTAAAGAAAGAAACAGTTCATCCTAATTGGC
CTGTGCAAATAAAGTCATATGACGAAGCTAAACTATCTTGGCAAGTTAAAGTTATTGATG
GTAAAGCTTGGGTAGGTATGCCATTTGAAGATTCTCAGGAATTTCGTATTTGGCTTAATG
ATGTAAAACGATATGTACATGACCAGAAAACTATGATATGTTATTATCGTCAAGAGCTAA
AAGAGGATAAATGTAAATGATTTCATGGTATCAATTTGAACATCTAAAAGGATTAATTTA
TGAATCCGAGATGGCTGCAATGATTTATGGACGACAGATTCAACGATTAGAATCTTTACC
TCCAACTAATGATGTTTTATTAGCTCAATCACGCGCTAATCTCAAAAATGAATATCAAAA
TAAGTGGGGTAAAGCATCTAAAGACCTGCATGATTATATTCAATCATTGGTTGAGAAAAA
TAAATGAAAACTCTGTTAGAACGTTATATTGAATGTTCAGACCGTTACATTGATGCATGC
CGTGAAAATGTATCAAGCATTAGCCAAGATATTGAACATGCTAAAGCTTTAGATGATGCT
GGTAAAGCCCTGCGAAAAGAAGCGAAAGCTCGTGGGCTTGATATGTATCAGCTTAAAAAT
CACATGATAAAATTTATTTCATCCAATGTTCAGAGTAAATCAGCGAATCAATCAACAGCT
GAATTGTACAAAGGACGGCGTGAGCATAATATTCGTATTCTTGAAGTTTTCTTAGGAATT
AAATGATGAAAAAGATTATTTTGACTGTAGGTTGTCCTGGTTCTGGTAAAAGCACTTGGG
CTCGTGAATTTATTGCTAAGAATCCTGGGTTTTATAATATTAATCGTGATGATTATCGCC
AGTCTGTTATGGCACACGAAGAACGTGATGAGTACAAATACACCAAGAAGAAAGAAAGCA
TCGTGACTTATATGCAACACGATGCGGCACATATGATTCTCTGCCAAGACGGAACGAAGG
GTGTAATTGTTTCAGATACTAATCTGAATCCTGAACGACGTAAGGTCTGGGAAGAGTTTG
CCAAAGAGCTTGGGCATCAAATTGAATATAAAGTGTTTGATGTTCCTTGGACTGAATTGG
TTAAACGTAACTCAAAACGTGGAACTAAAGCAGTACCAATTGATGTTTTACGTTCAATGT
ATAAAAGCATGCGGGAATATCTCGGACTTCCGGTATATAAAGGGACTCCTGGTAAACCAA
AAGCAGTTATTTTTGATGTTGATGGCACATTAGCAAAAATGAATGGTCGTGGTCCTTATG
ACCTTGAAAAATGCGATACCGATATTATTAATCCAATGGTCGTTGAACTAGCTAAGATGT
ACGATAAGCAAGGATATTACATCGTAGTCGTTTCAGGCCGTGAAAGTGGAACTGAAGAAG
ACTCAACAAAATATTATCGTATGACCCGTAAATGGGTTGAGGACATTGCTGGCGTTCCAT
TAGTTATGCAATGTCAACGCGAACAAGGTGATACCCGTAAAGATGATGTAGTTAAAGAAG
AAATTTTTTGGAAACACATTGCACCACATTTTGATGTGAAATTAGCTATTGATGACCGAA
CTCAAGTAGTAGAAATGTGGCGACGCATCGGTGTTGAATGCTGGCAAGTCGCCTCGGGAG
ATTTTTAATGGCTTTGCACCATGAAACTTGGGCTATTGTTATTGTAAATAGCGGTTTAGT
TGGTACTAGTAATGGGCAATTTTGTGTATTTACTAGTGAAAATAGAGCATGGGAGGAATG
TCTTAAATTAAGAGAAAAGAATCCTGATGTTGAACTAACAGTAAAGAAAACTAAACTGCC
TTTACCATGGAAAACTTATGAATAACATAGAAAAGATTTATCGTCTTTGTGATAAAATTG
AAAAAGAAAAGAAATATCTATTTTGTTTATGGCCTATTGTTGATGGAAGAGTAGGTTTAG
ATGTTCTTGATTATGAAACAGAAGACAAAGTAGATGGTTCAACTTTTGATAATGCGTTGG
ATGTTATTGATTGGCTCGAAGAAAATTATGTGAGGTAAATATGTTTCCGACTTATTCTAA
AATTGTAGAAGTAGTGTTTAGTCAAATTATCGCTAATAATATGTTTGAAAAGCTTGATAA
CGCAGCCGAGCTTCGAATTCATGCTCAAGTGACTCATGTATTGAACACTTTGCTTCCAGA
CCAGGTGGATTCTGTTGCCATCACGTTGTATCCAGGTTCCGCGCATATCATTGTTGTATT
CGGTCTTGATGCTGAGCTAGTTATCAAAGGCGACATTCGTTTTGAATCACAGACATCAGA
ATTCAAAGCAATTTAATAGTTTACTTTACGGTAGAGTTGTGATATTATAGCTCTACCAAA
ACAAACGAGGAAATTGAAATGAGCGAATGGTTTGAAGAAGATAAGGTTTATCGCTTTAAA
ACTGGGTATAAAGATATTTTTAATGAAACTTGCGGGGCTAATAAACGAATTGCCCAGTTT
ATTGGGGAAAATTCATTTAAAGTAAAAATAGATCCTGCGAAAAATGTTATTAGCATTAAA
CGCGAAATTGATGACTGTTGGTATAAAGCTGTTGATGTAATGGGCGAATCCTATAAAGTT
AGCCCGTTATTTTCAATTGCTTATATGTTAGAATATTCTTTTTTCGAAGAAGTTCAAAAA
GATGATTCTGTCAGTAAATTTGAAATTAAAACTGATAAAGAAATTAAGTGGAAAGTAGTA
GGTATTACTGGTTGTATGTTCTATATCTATGCTCAAACTGATACGAAGGAAGAAGCTAAA
AACAAAGCTCTAGAATATCTTGAAGAGTATGAAGAAGGCCCGGTAATGATTACCCAAGAT
GCTGAATTAGTTTCTGTCAAATTAGTTAAAAACGTTGAAAGTAAGGAGCTAGGATCAACA
TGCTAAGCGAAAAACCAATTACTGTTAAAGAATTCCAAGAAAAAGTTAAACTATTTGCTC
AAGAATTGGTAAATAAGGTTTCTGAACGATTTCCAGAAACATCGGTTCGTGTTATTACAG
AAACTCCTCGTTCAGTATTAGTAATTGTGAATCCAGGTGATGGTGACCAAATAACACATC
TCAAATTGGATTTTGATGGATTAGTTGAAGCACAAAGGGTGTATGGCGTACTATGATGAA
TTTAACTGATATAATTGATAATTGTCTTGAAAATGATACTGGCGATCATAGAGCGCTTGA
CTCTGAAACAGCTCAGTTCATTAGAATAACTTTAATGAATGACACTCTAGTGAATAGTAT
TCATCCTTCTGTGTATGATGCTATTATTGTGACGAAGTATCCAGTTGAGCTTCACAAAAA
GATGGTTGGCGCAATTTTTATTGATAAGAAAAACCGCTTTAAAGATGGGCAGAATATAAT
TAGTTCTGTTATTAAAAGTATAACTAAACTTCGTCACGAAATTTATCGTGTTGAAACTGC
TAAATCTGCTTATCTGGTGATTATGAAATGAAAGCGAGTACAGTACTTCAAATTGCATAT
TTAGTATCACAAGAATCAAAATGTTGCTCCTGGAAGGTAGGAGCAGTAATTGAAAAGAAT
GGACGTATTATTTCTACCGGATATAATGGTTCACCTGCTGGTGGTGTGAACTGTTGTGAT
TATGCTGCTGAGCAAGGTTGGTTGCTGAATAAGCCTAAACATACTATCATTCAAGGTCAT
AAGCCTGAATGTGTATCGTTTGGTTCAACTGATCGTTTTGTCTTGGCGAAAGAACATCGT
AGCGCTCACTCTGAATGGTCGTCTAAAAATGAAATTCATGCTGAACTAAATGCAATTTTG
TTTGCTGCACGAAATGGTTCTTCTATTGAAGGTGCTACTATGTATGTAACACTTTCTCCT
TGTCCGGATTGTGCAAAAGCCATAGCTCAATCTGGAATTAAAAAACTGGTTTATTGCGAA
ACATATGATAAGAATAAACCTGGCTGGGATGATATTCTGCGAAATGCAGGTATTGAAGTG
TTTAATGTTCCTAAGAAAAACTTGAATAAGTTAAACTGGGAAAACATCAACGAATTCTGT
GGTGAATAATGAAATTTCGTTTGGTACAGCTCACAGCAATTAGTTCTTATTCTAACGAGA
ACATTTCATTTGCTGTAGAGTATAAGAAGTATTTTTTCTCTAAATGGAAACAGTATTATA
AGTCAGAATGGGTTTGTATTGATAGGCCGTATAGTTGGAAATCTGATTTAGAAAAATGCC
AAAAATTACTTTCCACCCTTAAAGAACGTGGAACAACTCATATTAAAACTGTAATAGGTA
AATAAATGAAATTGACGACTGAACAGAAAGTAGCAATTCGTGAAATTTTGAAAACTAAAT
TGTCCATGGGTATTTCAAACGTAGTTTTTGAAAAGTCTGATGGTACTATTCGTACTATGA
AAGGTACTCGCGATGCTGACTTTATGCCAACCATGCAAACTGGTAAATTGACTGAATCTG
CTCGGAAAGAATCTACTGATATGATTCCAGTATTTGATGTTGAGCTTGGCGAGTGGCGAG
GTTTTTCTATTGACAAATTGATTTCTGTTAATGGTATGAAAGTTGAGCATTTACTCCAAT
TTATTGGTAAATAAATGCTTTAAGAATTATTTGTTATTATTAATTCATCTGTTAACAAAA
AGGAAAAACGATGTCTGAAGTACAACAGCTACCAATTCGTGCTGTCGGTGAATATGTTAT
TTTAGTTTCTGAACCTGCACAAGCCGGTGATGAAGAAGTTACAGAATCAGGACTTATTAT
CGGTAAACGTGTTCAAGGTGAAGTTCCTGAACTATGTGTAGTTCACTCTGTCGGTCCTGA
TGTTCCTGAAGGTTTCTGCGAAGTTGGTGATTTGACTTCTCTTCCAGTTGGTCAAATTCG
AAACGTTCCGCATCCTTTTGTAGCTCTGGGTCTTAAGCAGCCAAAAGAAATTAAACAAAA
ATTCGTTACCTGTCACTATAAAGCTATTCCGTGTCTTTATAAGTGATATAAATAATAATA
TGAATTGGGTGTCGGAATAATAAGTTAACCGAACAATTCTATGTGGTAGTCTACAACTGA
GAGATCTGTCGAAAGAAGATGAAATTCAGAAGAACGTGACTACCGAGTTTTAATCTCTAA
CGAGAATTTTTAAATGATTAAACAATTACAACACGCTCTTGAACTGCAACGAAACGCATG
GAATAATGGTCACGAAAACTATGGCGCATCTATTGATGTTGAAGCCGAAGCTCTTGAAAT
CCTGCGTTATTTCAAACATCTGAATCCTGCTCAAACTGCATTAGCTGCTGAGCTTCAGGA
AAAAGATGAACTTAAATATGCTAAGCCTTTGGCTTCTGCTGCACGAAAAGCAGTTCGTCA
CTTTGTGGTAACACTGAAGTAATTCATTGGAGATTCACTGCCTTAGTGTGAGCTAAAATC
GAGGAGCCGTCGAACTGTCTGATTAATGATTTGCGAATCATTATAGTTTTAAGACCCCGG
CAGTTTTACGGTGTACCTCTTGAATGTTATTCTTATAGCGGCAAGTGCATGCTACCCCGA
GGTGATGGCCAATCGGGAGTACGCCTCAAGGCCTATATATCCATCAGTATATATATCTTA
TCCTCGAGAAATCGAACCCGGAACCTTTAAGCTAACGGTGTGCAACAGATAAGAGCTATA
AGGTATGATGACGGGTTTATGGTTATCCTGGTCGTTAAATATCCAAAAACCTATGTTCCC
CTTGAGGGCTTGCGCAGGCAATGCCAATAAGTCCTGCATTTTCATTTAAAAGAGAATTTA
TAATGGCAAAACAAGCTAAAGCAAAGAAAGCAGTTGAAAAGAAAGTTGGTGATTCTAAAC
GCGCTGGCTACAAGCGTGGGTCGAACTCTCGTATCAATCAAACTGTTGAGAAGATCATGC
GCCGAGCACGTGCGGTTCTTCGAGATGATGCTTCTCGTTTTGGTAAGCAGAAAGCATAAG
TTGAGGACTCCTTCGGGAGTCCTTTTTTATTTTCCAAAGATTGCATAAAGTTGTTTACAG
TATGGTTCCTTTGTGATAGTATTATCTTACACAAACAAAGGAGAATAAAATGAAAACGAT
TAATCTGAACGCTGCAGTTAAAACTAAATGCTTCAATGGTAAATATGATGAAACTATGTG
GTTCTTAATGGCAGTTGAAGGTGATATTATTGAAGTAGAAACAACAGAAGGTATGGGAAC
AGATTTCACCTTTACAATTCAAGTTCATAATTTCTTTACTGGTTGGATTTATGAATTGAA
TACAGTAATCGTTGGAAAAATTGAACAAAATGAATTAGGTGAATGGTATTATGTTACAGC
CCGTCAACGTGCCGAACGCTTAATTGAGAAGATGAAAAAAGTTGGTAAACTTGACATGCA
GCATTGGAAAGTAGTAAAATAATTGTTTACTTTGGTACAGGACATGATATTATATACCTG
TACCGCAATTAAACATCTTGGAGAATAAAATGAACTACATTAACTTTGAACGTAAATATG
TTTCTAATGGTATTGCAGGTTCTATTGATACTATTTGCCTTTGGAAACATCAAAATGGAT
CAGTATGCGAAATTGAACAGTATATGACTCCTAACTACGTTTATATGCGATTTGAAAATG
GCATCACGGTTTCAATCACAATGGAAGGTTCAAACTTCAAAATCGCTCTGGATGATGATT
TTCGTCAACGCGATTTAGGGACCCATCCTTGCTGGAATGGCGCTAATCGTAAGCTTCTGG
TTAAAACTTGGATTCGTCATATTCTGAGTAACAGAGCTAAACCTGAGCATCTTGAAGCAA
TCTTTGATGTAGTTCTTAACGAATTTGATATTTAAAATAAAATGAGGGGCTTCGGCCCCT
ATTGAGGAAAATATTATGTTTATGACTACTTATTTTGATACCCGCAAAAATTTCTGTGAA
GTTGTTTTCTCAAAGGCACCTAAAGACCTTCCTGCACATTTGCAACCTACTAGCGAATCG
ATTAAAAACTACGTTGATGTGGTTTGTCCTTTAGAATTCCGTACAGTAAATGGGCGTGAT
ACTCTAGCTATCGCTAAACTCAATCGTGAAATTGACATTGATCCCTCAATTGCACGCGAA
ATTAATAGTTCTGATATTAATGGCGGTAATGTTAAATCACATGGGTTTCAGATGAGGTTC
TAATGAAATTCTTTTTAGGTCAAACTGTTGAATTAAAGGGAGTTGGTATACCTGGATTAA
TTTCTAAGGTTTTACCCGCATTTAAATTGGGAGATGTTCAAGTAAAAGAGGCTTATATTG
TTTCCTGGGTAGATGGAAATGAAGACCTTCGTATGGGTGATGAATTATCTCCTATCTACG
GATTAAAGGAATTAGTATGAATATAATTAATAAGATTTTTGGAATTCAGTACATTAAGGT
CACATATAAAGTAACAGATAAAAATCCGTATACTGATGAACACGAAGAACCACAAGTTGA
GTCTATTATATTAGAAAAGGACCCTAACTGGCCAGTTGAATTTCGTCTTCCTTGCTATGG
TCATTGGGCTGATGTTGAAATTATAAGCATTGAAAATGTCTGAGTTAGAGATTAGAAGCA
ATTTTAGGTGGCCATCATGCGCATTAAGTAATTTCGCCCATTGGCCTTTCGTTATGGATG
GTATTCAATTTGGAGGTCTTGAAGGATTCCTCCAAGGATGCAAGGTGAAAAACGTTGAAC
AACAACGTCGTATATTTGGGTTATCTGGGCTTGCCGCCCAACAAGCTGGAAGGTCTTATG
CTAGAGCTCAGGACCGTGGGACCCTCTTCTGGCTTGGAGTTCCATTTTCAAGATACTCCC
CAGCGTGGAAAGAATTATACACAAATGCATATTTTGAAGCAGCGATCCAAAATAGGGGCT
TTCGCGATGCATTACAAGCCTCGAAAGGAAAAGTTTTGAAGCACAGCATGGCTAGTGGTC
TAACAAAAGATGATACAATACTAACCGAAGCTGAATTTATTGATGTGTTAAACCTATTAA
GAGACTCTCTATGAAGCCTACTATTTTAACTGATATTGATGGAGTATGTTTAAGCTGGCA
ATCAGGCCTTCCTTATTTTGCCCAGAAATATAACCTTCCGTTGGAACATATTTTAAAAAT
GATCCAAGATGAGAAATTTATTTCTCCAGGTAAACTTTTTAATTGTGATGAAGAACTTGG
CGTCAAGTTAATTGAAAAATACAATCGTTCGGATTTTATTCGTTACTTGTCTCCATATAA
AGATGCTCTGTGTGTAATTAACAAATTAAAAGAAGATTATAATTTTGTAGCTGTTACAGC
ACTGGGTGATTCTATTGACGCTCTGCTGAATCGTCAATTTAATTTGAATGCTCTTTTTCC
TGGTGCCTTCTCAGAAGTAATGATGTGTGGTCATGATTCTTCAAAAGAAGAGTTGTTCAA
AAAGGCAAAAGAGAAATATAACGTAATTTGTTATATTGACGATCTCGCTCACCACTGCGA
TCACGCGAGTGAAATATTAAATGTTCCTGTTTATTGGATGGCTCGAGGAGAGCGTGACAA
TATTTCAAAAACTGCTCAGCGAGTTTATACATGGAACGATGTAGAGAATAAGCTTTTTTC
ACCAAAGGAAAATAAAGAAAGCTTTGATAGTGAAAAAGCTATAAAAGATGCAATCGAGAA
GATGATCAAAAACGATTCTTTTCCTTGGAACACTACCTGGAGAACTCCTGGATTTAATCC
TTATAATCATCTATATCATCCATATCAGACACATCCGTTTCAGACATGGAACTATATTAA
ACCTGGTGGCATAGAGTATTTGTATAATAGACCTACTAGTGGTGATAATATTTTCCAAGG
AGCATTCTAATGTTTGTCGTTCACACTATTTATGAAAATGAAGGTAATACTACACGTGAT
TATGGTCACGTAAATCAATTTTTTAGATGCAATCCGGAATTCCGAGCTCAAAAAGACGAA
CGAATTTTTAAAAAATGTGTAGAGCAAGGTTTCATTTACGTCAAGCACTGGATGCAAGGA
AATAAAGTTAGAACTACATATCACAGGTCTTTGCCTGAGCTTAATGATGAATTGATTTAT
AATAGAAATGTAAATCAAACTCTGAAGGATGAACAATGATTCTTAAAATTCTGAATGAAA
TAGCATCTATTGGATCAACTAAGCAGAAGCAAGCAATTCTTGAAAAGAATAAAGATAATG
AATTGCTTAAACGAGTATATCGTCTGACTTATTCTCGTGGGTTACAGTATTATATCAAGA
AATGGCCTAAACCTGGTATTGCTACCCAGAGTTTTGGAATGCTGACTATTACCGATATGC
TTGACTTCATTGAATTCACGTTAGCTACTCGGAAATTGACTGGAAATGCTGCAATTGAGG
AATTAACTGGATATATCACTGATGGTAAAAAGGATGATGTTGAAGTTTTGCGTCGAGTGA
TGATGCGAGATCTTGAATGCGGTGCTTCAGTATCTATTGCAAATAAAGTTTGGCCAGGTT
TAATTCCTGAACAACCTCAAATGCTGGCAAGTTCTTATGATGAAAAAGGCATTAGTAAGA
ATATCAAATTTCCAGCCTTTGCACAGTTAAAAGCAGATGGAGCTCGGTGTTTTGCTGAAG
TCCGAGGCGATGAATTAGATGATGTTCGTCTTTTATCACGAGCTGGTAATGAATATCTAG
GATTAGATCTTCTTAAGGAAGAGTTAATCAAAATGACTGCAGAAGCCCGCCAGATTCATC
CAGAAGGCGTGTTAATTGATGGCGAATTGGTATATCATGAGCAAGTTGAAAAAGAGCCAG
AAGGTCTAGATTTTCTTTTTGATGCTCCTGAAATCAGTAAAGCTAAAGAGTTTGCCGAAG
TAGCTGAATCTCGTACTGCATCTAATGGCATCGCCAATAAATCTTTAAAGGGAACCATTT
CTGAAAAAGAAGCTCAATGCATGAAGTTTCAGGTCTGGGATTATGTCCCGTTAGTAGAAA
TATACGGTCTTCCGGCGTTTCGTTTAAAATATGATGTACGTTTTTCTAAACTAGAACAAA
TGACATCAGGATATGATAAAGTAATTTTAATTGAAAACCAAGTAGTAAATAACCTAGATG
AAGCTAAGGTAATTTATAAAAAGTATATTGACCAAGGTCTTGAAGGTATTATTCTCAAAA
ATACCGATGGATTATGGGAAAATGCTCGTTCAAAAAATCTCTATAAATTTAAAGAAGTAA
TTGATGTTGATTTAAAAATTGTAGGAATTTATCCTCACCGTAAAGACCCTACTAAAGCAG
GTGGATTTATTCTTGAGTCAGAGTGTGGAAAAATTAAAGTAAATGCTGGTTCAGGCTTAA
AAGATAAAGCCGGTGTAAAATCGCATGAACTTGACCGTACTCGCATTATGGAAAACCAAA
ATTATTATATTGGAAAAATTCTAGAGTGCGAATGCAACGGTTGGTTAAAATCTGATGGCC
GCACTGATTACGTTAAATTATTTCTTCCGATTGCGATTCGTTTACGTGAAGATAAAACTA
AAGCTAATACATTCGAGGATGTATTTGGTGATTTTCATGAGGTAACTGGTTTATGAAAGC
TTACTTAGAAACAATTGTCATTGCTCAAAAAGAAGGTGGAGATGTTTCTACTTCTGTATC
ACAAGTCATTCTCGAATTTGTAGATGCATACGCTTATAATAAATTTACAGAAACATTTGA
TGCCTATGAAAAAGGACCAAAGTTTGAAATATATCGTACTCTCTTACCACTAGATTACTA
AAGGCCTTCGGGCCTTTAATTTTATAAATAGAATAAACACTAGAGAGGCTATGATGGAAC
TTATTACAGAATTATTTGACGAAGATACTACTCTTCCGATTACAAACTTAAATCCAAAGA
AGAAAATACCGCAAATTTTTTCAGTTCATGTTGATGATGCAATTGAACAACCAGGCTTTC
GTTTATGTACCTATACATCTGGAGGTGATACTAATCGCGATTTAAAAATGGGCGATAAAA
TGATGCATATTGTTCCTTTTACATTAACTGCTAAAGGTTCAATTGCTAAATTAAAAGGTC
TTGGTCCAAGCCCAATTAATTATATCAATTCAGTTTTTACTGTTGCAATGCAAACAATGC
GTCAGTATAAAATTGATGCTTGTATGCTTCGTATTCTTAAGTCTAAAACCGCTGGTCAAG
CTCGACAAATTCAAGTTATTGCTGATAGACTTATCCGTAGTCGTTCAGGTGGCAGATACG
TCCTTCTTAAGGAACTCTGGGATTATGATAAAAAGTATGCATATATTCTTATACATCGCA
AAAATGTATCACTAGAAGACATTCCAGGAGTTCCGGAAATTAGTACCGAGCTCTTTACTA
AAGTTGAATCGAAGGTCGGTGATGTTTATATCAATAAAGATACCGGAGCTCAAGTAACCA
AAAATGAGGCAATTGCAGCATCTATTGCACAAGAAAATGATAAACGTTCTGACCAAGCTG
TAATCGTTAAAGTTAAAATTTCCCGTAGAGCAATTGCACAAAGTCAGTCATTGGAATCTT
CTAGATTTGAAACACCGATGTTTCAAAAATTTGAGGCTTCAGCGGCAGAATTAAATAAAC
CAGCGGACGCGCCTTTAATTTCTGATTCTAATGAATTAACGGTAATTTCTACTTCAGGAT
TTGCACTAGAGAATGCTCTTAGCAGTGTTACAGCTGGGATGGCATTCAGAGAAGCTTCTA
TAATTCCTGAAGATAAAGAATCCATTATTAATGCAGAAATAAAAAATAAAGCTTTAGAAA
AATTACGAAAAGAATCTATTACTTCAATAAAAACTTTAGAAACTATTGCTTCTATCGTCG
ATGATACTTTAGAAAAATATAAAGGTGCTTGGTTTGAAAGAAATATTAACAAACATTCGC
ATTTAAACCAAGATGCTGCAAATGAGTTAGTACAAAATTCTTGGAATGCAATAAAAACAA
AGATTATTCGAAGAGAATTACGTGGATATGCTCTTACAGCTGGATGGTCATTACATCCTA
TAGTCGAAAATAAAGATTCATCTAAATACACACCAGCACAAAAACGCGGAATTCGTGAAT
ACGTAGGTTCAGGATATGTAGACATAAATAATGCTCTTTTGGGATTATATAATCCAGATG
AGCGTACAAGTATTTTGACAGCATCTGACATAGAAAAAGCTATTGATAATTTAGATTCAG
CCTTTAAAAATGGTGATCGATTGCCAAAAGGTATTACTTTATATCGTTCACAGCGAATGT
TACCTTCAATATATGAAGCATTGGTAAAAAATCGAGTTTTTTATTTTAGAAATTTTGTAT
CAACATCATTATATCCAAACATTTTTGGTACTTGGATGACTGATTCAGCTGTTGGTGTTT
TACCAGATGAAAAGCGTCTAAACATTTCTATCGATAAAAATGATGAAGGGCTTGTTAATT
CTAGCGATAATTTAGTCGGAATTGGATGGGTTATTACTGGGGCTGATAAAGTCAATGTTG
TTTTACCCGGTGGAAGTTTAGCGCCTTCAAATGAAATGGAAGTTATTTTGCCACGTGGAT
TAATGGTCAAAGTTAATAAAATAACTGATGCATCTTACAATGATGGAACAGTTAAAACTA
ACAACAAGCTTATTCAAGCTGAAGTTATGACCACGGAAGAGCTCACCGAATCGGTAATCT
ATGACGGAGACCATTTAATGGAAACTGGTGAATTGGTTGCAATGACAGGTGATATTGAAG
TAGAAGACAGAGTTGACTTTGCATCATTTGTTTCATCAAATGTTAAACAGAAAGTAGAAT
CATCTCTTGGAATTATTGCGTCTTGTATAGATATTGCAAACATGCCTTACAAGTTCGTTC
AAGGATAAATCATGGAACTTATTACAGAATTATTTGACGGCGCTTCGGCGCCGGTTGTTA
ACTTAAATCCTAAGCATAAAATACCACAAATTTTTGCTATTCAAGCCGGCGAAGAAAGCG
TGCTTCCTGGATTTAGATTTTGTACATACACCTCTGGCGGTGATACGAATAAAAACGTTA
AGCCAGGTGACAAAATGATGCATATCGTAATGATAGGTGTCAATGAGAAACTATCATTAG
TTAAACTTAAAAACTTGGGTGGAAATCCAATTGGTGTCATTAATGCGGTTTTTGATACTG
CTCTTCAAACAATGAAACAGTATAAAATTGACGCATGCCTATTTCGCGTACTAAAAAGTA
AAACAAATGGCGCAGCTCGTCAAATGCAAGTTATTGCTGACCGTTTAGTACGTACTAAAG
GAGCAGGTCGATATGTTCTTTTAAAAGAAATCTGGGATTATGATAAAAAGTATGCATATA
TTATGGTTTACCGTAAAAATGCCAATTTAGAAGACATTCCAGGTGTACCTCCTATTTCAA
CTGAGTTATTCACAAAAGTTGAATCAAAGGTTGGTGATGTTTATGTAGATGTTAAAACAG
GTAATGCTGTTCCTAAAGCTGTCGCTGTTGCTGCTTCTATTGCTTTAGAAAATGATAAGC
GCACGGATCAAGCTGTTATTCAAAAAACTAAAATTAGTCGTCGATTAGCAGCACAAGCTC
AATATTCTACTGTTGATGCTTCACTTCAGGGTGATAGCTTCGCTGCTAAGAAATATCAAG
AGTTTGAATCTAAAGTTCCGGTGTATAAAGCAGAAGGACCAATGAACTCTGGCGTTATTC
AAATTGGTTCAAACTTCAGCAAAGGAGCTATAGGCGGTATGAGAAGCGCTTCTCGTTTTA
AATCTAGTGATTATGAACTCGAAAGCTTCCGAAATCATATTGCATTAGCCCATGCACGTT
TACGTGATCCATCTATCAAGTTACAGAGCGATATAACATATCAAGGTTCTCAAGAATATT
TAAAGAATAAAGAATTCTTTGATTATAAGACTGATAAAATTTTAAGCGATCTTGCTGATA
TTAATATTTCTAATAGCTTTGATGTTATTAAGAAAATTATCAATGATTTGGTTAAAGGTT
CTAAAGCTACGCCAGATGAAAAGACAGCTATTATTCAATTTGTCATGAATGGCATTTATA
AATTGATTAATGAATCTGCTGCTCAGGCATATGAATACGCAAGCACTGAAGTAACTCCAA
AAGGATTAACTCAAGCTGAATCTGATGTAATTGAAGATTATTGTGCAGATTCATATGTTG
AAATGAACTCGTTCCTTTTGGGTAAACCAGATTCTACCCGTGAAGAATATATGGAACGAG
CTATTAAACATATTGAGACGCTTGATTCTGCATTCGCTAAAGGTTCAGTTCTTCCTCCAG
GAACTACACTTTACCGTGGACAAGAAGTTACCTTTAAAACTTTGCGTCATAACATTGAAA
ATAAAATGTTCTATTTCAAGAACTTCGTATCCACATCGCTTAAACCAAATATCTTCGGTG
AGCATGGTAAAAACTATATGGCTCTAGATGATTCCGGCGCAGTATTTTCTGGTGAAGGAG
AAGGTTCCGTTGATGCAGAAGATTTGATGCATATGGGTAGTCATTCTACATATGCTAATG
AAGATGCTGAAACTAGCGTGGGTATGGTAATTAAAGGAGCTGAGCGAATCAAAGTTATCG
TTCCGGGTCATTTATCAGGATTTCCGTCAGAAGCTGAAGTTATTCTACCACGTGGAATTT
TACTGAAGATTAATAAAGTAAGTACTTACTTTATGAAAGAAACTGCTTATAACAAGTATC
TAATCGAAGGTACAATCGTTCCTCCTTCTGAACAATTAGAAGAATCAGTATATGATGGCG
ACCATCTAATGGAAACTGGTGAAGTTCGTCCAATGGCTGGATTTAATCAATTCCTTGTAG
AAGAATCAAAAGAAGAGGAAAGCGAAGTTTCTCAAATCTTAGCTTCTTTGGTTAACATCA
ACGGAATGTCTAAAAAGTTCAAAATGTAGTTTACAAGTCCCTCGTGTTGTGTTATAGTAG
TCTTACTGACATAACATGAGGAACACAAAATGAAATCTTCTTTACGCTTTTTAGGTCAAG
AACTTGTAGTTGAAGGCGTTATTCCTGCTGATAACGCTTTTAATGAAGCAGTTTATGATG
AATTTATCAAAATTTTTGGAACAGATAAAAAGTTCGGAATTTTTCCTTCTGAAAATTTTT
CAAAGCCAGAACAGACTGAAAGTATTTTCCAAGGTGTAGTAACAGGTAAATTTGAGTCAG
AAGCTCCGGTAAAAATTGAGGTTTATATTGAAGAAACCTCTGTTGCTTCAGTTGCTGCTT
TTATTTCATTCCGTAAATAAAATATGGGGACCTTTCGGTCCCCATTGTTATATTGCTCCT
AATATTTTACTTTGCGAATTGACAATTCCTGTCATAGTATTAATATTTGAAATGCTTCCT
GCAGTTCCCCCTAATCTACTAAGTCGTGAAAGCGAATTAGATAGTCCAGTAACACCTCCA
CTATTTCCGAGAACGCTTTGAATACCATTTATAGTAGCAGATTCAAGCCATTCAATGGCA
GCTTGTCTATCAACTGCTCCAGCCTGCATCACTCTATACGCAAAAGTAACATCAAATGTA
GTTATTTGGTTATCTCCATCATATGATAACTCAGGAGCGCTTACGGACACTGGAATGCAT
CCAGTGAACATCACCGCAGTATGTGGTAATCCATTGCGAGAATGAAGATTAACCTGAATA
TCCGCCTCGACATCTTGCGGCAAAGCACGCAATCCGGTTACTGGGTCTTGAACAGCGTTA
ACCCAGTCTTGCATTGCACGATAGTTACAAGCTTCTGAATCCATTCTGAATGAAATAACC
AAAGGGTCTAATTCTCTCCCAGTTATACGAATATTAGGAGAATTATAGTTCCAGTCAGTT
TCATAGGATAATCTATTCTCTGGCATTTTTACAGAGTATATCATCAATCCAGATGAGTTA
TATGCCACGTTAAAGAAGTCAATTAAATATGTACCAACTGTAAATGAGCCTAATAAACTT
TGAACTGTACGTTGACTCATGGCACCAATAAGATATTTACTAACCCCTGATTTTCTTATC
AGTTTTTGTGTGCCAGCAGTAATTAGCGTGGTAATTCCCTGATTAATATCACCTTGAGAC
AATCCTAACCAGTCTGAATTTAGACCCAAGTTATTATAAGAAAAGTTGCTAATTGAACTT
ATCAACGAAGAGCTTTTAGTTGATGGAGTTGTCGCAAAAACGCAGCTAAACATATTATTA
CGTTGAAAATCTGCGTTTATTGCTTGATTATTAAATTCCTCTAAAGAATACATTAGAAAG
TCCCCGTATATAAAGAAGCACGGTTTAACGTGATAATCTCTCTCATAGTAATCTCGAGTG
TAAAAGAACTTGGAAGGTTAGGAGCAATAGCTAATCCGTTAAAATTACCATTCGGAGTTT
TATCAAATCTAATACTTTGAATTTGACACGGTCCAAACACTTCTGTTTTTCCGTCAAATT
TAGAAGTTGCGCCAAAGTTTTTAACCATCCAAATTGTCGGGTTTGAAACTACAAGAACGT
TAGTTAAACTCGATGTCATTTTCTCAAATAACGTCTTATTTTTAACTGCATCTTCTGGAG
TTAATGGCTCAATAAAAGTAGAGCGATACCACTCATCTAGATATCCTTTTATTTCAGCAG
CATATTGAGATTTACCCGTTTCACCATAAGAAAAATAGTTAAAATATTGATAGATATTAA
TAATAGCCATTAAATCTTCTGTGGAACGCGGAGTCAAATCCCAAGTGAACACTTTAGTTC
TATTCTCAGCACCACCATACATACTTCTGGCTATCGTATAAATCTGTTCATTATTATCAG
CCATTATACCTTGTGTTATACTTTCCAGTGCTCCAAATACTGCAGTTGAAGCCATATTGC
TTAACACGCCAGTAGCAGTACCTCCACCTTTTGTAATAAGACTATCTTGAACATCGTTAA
ATCTATGTGATGATGTATCAACATCGGATTTAGATCTCGGTAAAAGAATATTTGCAACAG
GAACTTTACTTATTGTTCCTGAATTATTATCTGATATTAATCCATTTGATAGTTTTGATA
CTGTACTACTAATAGTGTTTCTGGCTGTACGTAAAATACTAGAAGATGAAGAAGAGTAGT
TAGATCTCATCGTTCTGAGACTTCCAGAATCCCTAGATGACATATTGTATGCAGTAAATA
ATAATCCATTCTTATATAGATCTGTTACCTGGAAGTCCCCTGTAGTGTCATTCCCACTAG
CACGCCCAGTTGGAAACTGGGCTGTGTATGTTTTAGTTCCTACTTCTGATTTAGTACTCT
GTCCGGCTGAAATTTTCTCACCGGACTTTTTAATTAAATCAGCAGTTATTTCTTTAACAA
TTGCCATATTATTCCTTAATTAACTCCCGTCGCACCAAATACACCAGGAGCAGTTGTGCT
TGTGACAGGTGTCATATTATGAACGACAGTATTTTTCTTAATAACATTATTAGTATTATT
GATTGAAGGAGATGCTTGTTGAACAGGAGCTTGCTGTGCTTTGTTCTTTTCTATTACCTG
GACTTGTTTTGCTTCTGGCGATTTAGCAGAAGTTTCAGGTTTGGCCTCAGGTTGATTTTT
CTTAAGCTCTTGATAAGTAGCATCAATTTTAGCAAATCTAGAAGCAAGCTCCTTTTTAAC
TGCAGGTGAATTATTTAAATCCGGGTCATTCATCCGTTTTTTAAGGTCTTCATGAGCGGC
TTCAATTGATTTAACTGTTGAGTCTTTACTCATATCAGCTGAATCAGCATATTTTTCAAA
ACGAATCATCGCGGCACGGGCTTCATTAGCCTTCATTAAAGCGTTTTTTCTTTCTTCTGG
TGAAAGTTGCTTTAATTTTTCTTCTTCTGCTGCGCGTTCTTCATCAGTTGTCAATGCTTC
TTTATTATCAACACCACGAATCCAGTTAGATGCACGGGTTTTCCAATTAGCAATTTTATC
TAATCCTTTTGCTATTGGACCAAGATCGTCATTCATTCGTTTATCTTGATAATTTGCTAC
TTTTTCTTGGTCTTCTTTATTGAGAGATGCTCCAGTAGAATTTTGGAAATTTTCTAATGC
TCTTCCTTCTACTTCATCAGCGGTATCCTTCATACCAGGAATAACTCGAAGAATTGCTGC
TGATAATTTAGCCATTCCAAGTTGAATAAGTTCTCCTAAATTATAAAGAACACTTCCAAG
TCCTTCCACAATAGCTACTGTCAATCCACCCCAGTCTCCAGCTTCCCAGAACTTTTTAAT
TTCATCAATAGATTCAAAGATGCTCTGTAATAAAGGACCCCAAGTTCCGGTTTCGCTTGA
AAAAGTTTTAAAATCAGTGCTAAACAAATCCCAGGCTTTTGAAAATTTATCTGACCAATA
TTTAAAATGAACCATCAGTAGGTCTATTCCAATAACAACAGCCATTATTAATGCAACCAT
TTTAGCAGCTTCAATAGCAGCACTGACAGTATACTTAAATAGCATGCTTGATATTTTATC
AGTAATTGAAATAGATTTCTTAAATCCGAAATCAACTGTCTTTGTTAATTTATCTAAAGC
ATTAGATATTTTTAAGTTAAATGCGTCTCTTTTTTGCTTCTCTTCTGGAGACTCTTGTTT
TGGTTCAATTGGCTGAGGAGTAGGGAAAAAATCAGCATCAGGATCATTATTAACTGCTTC
AGGAGCCGGTAATAAAGGACCCACAGATTCAGCTGTATCATCCTCGACGACTTTAACAGG
AATAGCATTTTCGACTACTGCTAAACTCGTTCCAGTTTGCTGAATTCCAGCTGTCTGAAT
TTTTTGTTCCAGTAAACTTGTTAATTTATCTAATTTGCTTCCGAGCGATTCACCGATTTC
TTTATTAATATTGTTTCCAATTTCAACAGTTTCAGCAATTAACTCAGAACCGGCAGTAGT
ATCACTTACTGCGCTTTCTACATTGTCAATTGCTCCAATTATTTCATTCGATTTTTCTTC
GACAGTTTGAGCAATTAATTCAGAAGCAGCTTGAGCATCATCCAATTTCGTAGATATATC
GTTAAGTCCAGATAAAGTATTGGAAGCGGATTTAGCCGCTTCCTGAACTGGTTTATTATC
TGAAATAACTTTTCTACGCATCGTTTGCATTTCTTGTGGCTTTTTCATTCAAATAATCCA
ATAATATTGCCAATTCCAGTTATTGAACCATTAGGGCCAGGAATTGCTAAAGTTGTAGAA
ATATCATTTGCCCATTTTAATACAAATGCTGGCATCTCAAGAAAATTAATTTCTTTAACT
TCATCATTGACCTTAAGCAAGCATTTGGATAACATATCGCTTACAGTTAAAAATTGTTCA
AATTTTCCAGGAGGTCTAAAATAAAATGTATTTCCTTGGTATTGAAATTCCAATCTTTGG
CATACATAAACGTCATTAATATCATAAGTATAACCATCTATTTCTTTACGAGATTTAATC
TTTCCATTAAATTCTAGCAAATGAATAGAAACGAAATCAACTTCTGCCGGTGATAAATTT
GGACAAATAGAATCAATAAGAAGCTTTAAATTTTCATCAGGACCTTTAACATCTTTTAAA
ATGTTATAATGTTTAAGACCCATCTTAGGAATAGAAACTTCTTTATTACTTATCGGAAGA
ACTACTTTCTTCAGTGGTAGTATCAGTTTTAAATTCATTTTTAACCTTAACTGGGTCGAT
TGTTTCCAGTTTCGTTGCATTAGTGAACATATAAAGATGAGTTACGGAATTATTATTTGA
TAATTCGTGAATAACTTCATCAACGTAAAATTCTGTTTTAAATTGGTTTTTACTATCATT
AAAAATAATTTTAACGCCAGGAGTCAAGTTAAAATTACCGACAGTAGAACATTTAGCGTA
ACCATCATACTGCGCCATAGTCTGAAGACGAATAGCTTCTTCATAGCCATTCCTATAAGT
CATTTCAGAATAAGCACCTGATCTGGACACTACAATAGAGTTTTCACCCTTTCCTGTAGT
AATCATTGGCAATGAAGAATCCAAAAACGAATGCGCATAGATAGTAGCATTTTTCATTGG
ATCGCGTTTGTGAGGATTCGATTTAGTCAACCAAACGAAATCATATGCTAATGGATATTT
TAATTCTTGGACAAATTGACCTATTAAAGTTGGCTCACCGACAATCATTGGATATGGTTC
TTGATTTATCATCATATCATAGTCCATCATGTTAACTCCCATGATGTCTTGCCATACAAA
TACGAACTTATCGCTTCCTACCGCTAGAGCAACTTCTCTTACATATGACAAATAGTTTTC
AAATGTGCTAGTCCATGGAATATCAGGAACATAAGCATTAATAGCATTTATCGCTGGAGT
TAATAATGTACGATCTTGATAAATTACGCCAAGCATTTCCTTTATAGATTCACCGGCATC
AGGGAAAAATGGTCTACCAAATTTAAGATTTTCTATAGAATGAATAGTTCCTAATTCAAT
AGCAATGATGTTATCGCCTTTTGAATCTACTGACACGGAAAAATGCTTGCACCCATAAAT
TCGGGTTTTAACATTATTAATATCGTTTGCATTAGCTACAGAAATCTGAATTATTTCATT
TCCATCCATTTTTGTATGAATGTTTTTAGAATCATAAAATTGTAGCATTCCTTCATTTCG
ACCATAAAGAGAGTCACGCATAGTTAATGTGGTAATAGTAGCAGCTAATTCAACAAATCT
ATTATTACTCCAAGCGTCATAACTCTCAAATAATTTAACGCTGAGATTTGGATATCCAGG
ACGTTGTAACATACTCATTGATGTTTATCCTTCTCAATCAGTTTTAATACGAATCCGCGC
TCGGCAGGAATCATTTTCATTATTGAATTTAAGCTATAATTACTTTTTACAAGCGTGTGA
TTAATTTGATAAAAAGTAAATATCTCATCTGGATTAACTAATAGCTTAAACACATCTACT
ATATCAGTGTATTTTTTAATGTACTTATCACAACACGACATGTGCAATGTTAAATTAATA
GGATTCATTGCATCGAGAATTTTTTCTAATGTTTCTATCTCGATGGCGTCAACAAGTTCT
ATTTGACTTGATTCACTAATTTCCTTCCAATCATACCACATTTCATCGACTTGAACAGAA
TGAATATTTTCAGTAATCATCTTTGCTTTATTTTCATAAAACTCAGAAGGAAAATTTAAT
TTAATTTTAACATTAGCTACATCAAAAATAGGTTCCTTTAATTCTTTTTGATATATTTCA
AATGGAACTGTCTTTTCTTTTTTACATTTTGGACATACAAATGTGACTGGTACTTTAGTT
TTACCTATTGACCCTACAAATACCTGCAAAAATATAAATGGTTGCCAAGTCTTTGGATAG
TCTCCAAAATAATCAGCAATTAAATCAGCAATTACTTCTTTTTGTTCTTGTGGTGACCGA
TGTTCTATATCGTTTCGAACTAACAAAAAATCTCGATAATCTTCTACCGTAAATGGTTTA
AAACGATGAACACCATCTGGTAATTTACAACGAATAATGTTTGCCATAGATGCTCCTTTT
ATTCTATTTATAAATATGATAAATAAAGGAGCTAAATATGTATGAATACAAATTTGACGT
GAGAGTTGGTTCTAAAATAATCAACTGTCGCGCATTTACTCTTAAAGAATATCTAGAACT
TATTACTGCTAAAAAGAATGGTTCAGTAGAAGAGATTGTTAAAAAGCTAATCAAAGACTG
TACAAATGCAAAAGATTTAAACCGCCAAGAATCAGAACTATTACTGATTCATTTATGGGC
GCATTCTCTTGGAGAAGTTAATCACGAAAACTCCTGGAAGTGCACCTGTGGAACTGAAAT
ACCAACCCATATAAATCTATTACATACACAAATAGATGCACCAGAAGACCTCTGGTATAC
ACTGGGTGACATTAAAATTAAATTCCATTACCCTAAAATTTTTGATGATAAAAATATAGC
CCACATGATAGTATCATGTATAGAAACGATTCATGCTAACGGTGAAAGCATTCCAGTTGA
AGACTTAAATGAAAAAGAATTGGAAGATTTATATTCTATCATCACAGAGTCAGATATTGT
AGCTATAAAAGATATGCTTTTAAAGCCTACTGTTTATTTGGCTGTTCCAATTAAGTGTCC
AGAGTGTGGAAAAACCCACGCTCATGTAATAAGAGGACTCAAAGAATTCTTTGAGCTATT
GTAATGGCAAATATTAATAAGCTTTATTCTGATATTGACCCAGAGATGAAAATGGATTGG
AACAAAGACGTTTCAAGATCACTTGGATTAAGGTCAATTAAAAATAGTCTTTTAGGAATT
ATTACAACAAGAAAAGGTTCAAGACCGTTTGACCCTGAATTTGGATGCGATTTATCTGAC
CAACTTTTTGAGAATATGACTCCTCTTACTGCTGACACGGTTGAGCGTAATATCGAAAGC
GCAGTAAGAAACTATGAGCCACGTATTGATAAATTAGCAGTTAATGTAATACCGGTTTAT
GATGATTATACTTTGATAGTAGAAATACGCTTTTCAGTCATCGATAACCCTGATGATATT
GAGCAGATAAAACTACAGCTGGCTTCCAGTAATAGAGTATAATGCTTCACGTATAAACGT
GGTATAATGAATCTAAGTCCATCCAAAATTGAATAGAGAACAATATGAGATTAGAAGATC
TTCAAGAAGAATTGAAGAAAGATGTGTTTATAGATTCGACTAAATTACAGTATGAAGCAG
CTAATAATGTGATGTTATATAGTAAATGGCTTAATAAGCATTCAAGTATTAAAAAGGAAA
TGCTTAGAATTGATGCACAGAAAAAAGTTGCTCTTAAAGCTAAATTAGACTACTACTCGG
GACGAGGAGATGGTGATGAATTTAGTATGGATCGTTACGAGAAATCAGAAATGAAGACAG
TTCTATCAGCTGATAAGGATGTTTTAAAGGTTGATACCTCGTTACAGTATTGGGGGATTT
TATTGGATTTCTGTAGCGGAGCTCTTGATGCTATCAAATCACGCGGATTTGCTATTAAGC
ATATTCAAGACATGCGAGCATTTGAGGCTGGAAAATAATGAGATATAGCATTGATGATGC
TTTTAATTATGAAGAAGAATTTGAAACGGAAATTCAATTCTTAATGAAAAAGCATAATCT
TAAGCGTCAGGATATTCGTATCCTGGCCGACCACCCGTGTGGTGAAGATGTCCTTTATAT
TAAAGGAAAATTTGCCGGATATCTTGATGAATATTTTTATTCTAAAGATATGGGCATTGA
CATGCATATGAGAGTTGTATAAATAGATATATAATTCAGAGGAGACAATCATGTCAGATA
AGATTTGTGTTGTCTGTAAAACTCCAATCGATTCTGCATTGGTTGTTGAAACAGACAAAG
GTCCTGTACATCCTGGGCCTTGCTATAATTACATTAAAGAACTACCAGTTTCAGAAAGTT
CGGAAGAACAATTAAATGAAACACAACTTTTGCTATAGTGTGACCTTTAGTCTATAGTTT
TGGCCCTTCCTTTTTGGTTGGGCCTTTTTTAATTTAAAAGCTTTCTTCTACATCATCGTC
TGAATCTTCTAATTCAGCTCTTTTTCCTGCCAAAGCATCTCTCACAGAGATGTCATCAGT
ATCTTTTAATTCAGTTTCTTTAACTCTTTTCTTATAATAAGCTTCAAGTTCTTCTAGACC
TTCTAATGTTTGGCAAGAGGCAATTTTACCCATAAATTCATCAATAGAAGCTTCATAAAG
AAATTGTTTAAATTCTAGTAACATCTTTTTCTCCAAAGGGCCGAAGCCCTTATAAATTAA
CTGTTTTCATTACGTAATTAAATTTTTCATCTGCGTAGCGCTGAATACGATCAATGCCGT
GTTTTAAAAGATAGTTCAAATGAACATATTTCTTTTTCGTATTAGCAGATTTTGGCTTAA
CACCACAATCATCTATGAGGTCCCAGACTGTTGCTATTGTCTTAGAACCATGCTTACGTA
ATACACGACCGATTGTTTGTAATACAATGATTTTTGATTTAACACCGTGCGCTAAAACAA
CGTGATGCAGATTTTTAACTGAAATACCAGTAGAAAATACACCATAACTAGCTACTATAA
TTATTCCTTTACCATTTTCAGCTAAGGTTTTCATTATATTGCGGGTTTCGGTATCAACTT
CCCCTGATACGTAATAAACTTTATCGTATTCATTTTTAATTAAATCGAAAATAGCTTTAC
CATGTGATACATGTTTAAACATGACAAAAGCGTTTTCATCTTTTTGCGCAAGCTTAATAG
CTAATTTAGCGATCCATTTATTTCTTTTACTAAGCCCCGTAATAATTTTTATTTCTTCTT
GATAAGTTTTTCCCTTTAATTTAGTAGTGAACTCATCGGGATAGCGAAGAAAAATACTAT
TAATTTTTAGCTCAGTTACTTGTCCATCTTCCATTAACTTAGAAGTCGTTACTGGCTTAA
ATATTTCACCAAACATTCCAACATACTGCATGATATTGGCTTTGCCATCACGTAATGAAC
CAGATAGACCAAATTTGAACATGCAGTTATTTAAACCTGATATGATAGATGAAATACTTT
TTCCTGTAGCAAGATGACATTCATCATTCATCATCATTCCAAACTGTGAGAACCATTCTT
TTGGTTGTTTAACTACAGTTTGCCATGTACCAACAACGACTGGTGCATCATTTTTATATT
TATCATCTTTTGATGCTCCGCCACCAATTTTCTTTATCATTGCATGACTGAATAAACGAT
AGTCAACGAAGTCATCAGCCATCTGAGTTGTTAGAGCAGTTGTTGGAACAATGATAAGAA
TTTTACCTTCGTAATTTTCCAAATAATACCGAGCGAGCAGAGCCTGAATTAAAGATTTAC
CAGCGGATGTTGGAAGATTAAGAATTCTACGACGATTAACTAATCCTTCGAACACTGCAT
CTTTTTGATACCAGTGTGGTTCAATTCTTTTATTTCCTGAATAGATTTCTAATTTAGAAA
GCCATTCATCAAAATCTTTTCTTGATAATTCTTCTTTTTCGTTAATTTGTGGGTCAATCC
AGGCTTTATAGCCAAAGTTATCACAGAACTTTTTAATTTGCCCGACTAAGCCGAATGGAA
GAAGACGATTATAATCTAAAAGACGGATTCGTCCATCCCAGTGGCCATATTTGTATTTCG
GATTAAAACGATACCCATCAGCTTCGAAGCTAAAGAAATCACGGAGTTCATGAAATGTAC
TTTCTTCACAATCAATTCTCACGTGACTAAAATCATAAAAATGTACTTTAATGTCAGTCA
TGTCTAAATACCATGTAATAAATATATCTATATTTATACTGAGGAAATATTATGATAGAT
AAAGATTATATTGCAGAGTTAAAGGCTCTTGATGATAATAAAGAAGCTAAAGCTAAATTA
GCTGAATATGCTGAGCAATTTGGTATAAAGGTCAAAAAGAATAAATCTTTTGATAATATC
GTTGTTGATATTGAAGAAGCCCTCCAGAAGCTCGCTAGTGAACCTATGCCAGAGACTGAT
GGGTTATCTATTAAAGACTTAATTGATGCTGCTGATGCTGCAGAGGGATTAAAATATGAC
GATGAAGAAGTTAATCCAGAAGCAGCACTTCTGATTGATTCTCCAGTTAAATCTGATATT
AAAATTGAAGTGGTAGAAACGGATAAAATTCCTGAAAATACTGATGTTTTGATTGAAGAC
ACTCCTTTTGTTGAAGAAAAGTTTGAACAAGCTGTAGATAAGATTATTGAATCTGAAAAG
CCGTCTGTATTTACTCTTCCGGAAAACTTTAGCCCGAATCTTCAGCTGATTGGAAAAAAT
CCAGGATTCTGCACTGTTCCTTGGTGGATTTACCAATGGATTGCTGAAACTCCAGATTGG
AAATCTCGCCCAACCAGTTTTGAACATGCGTCTGCGCATCAAACTTTGTTTAGCTTAATT
TACTACATTAATCGTGACGGATCAGTTCTAATTCGTGAAACACGCAATTCTTCTTTCGTA
ACATTAAAATAAGGATAACTTATGACTTTTACAGTTGATATAACTCCTAAAACCCCAACA
GGAGTTATTGATGAAATCAAGCAGTTTACTGCTACACCCAGTGGTCAAACTGAAGGTGGA
ACTATTACCTATGCTTGGAGTGTAGATAATGTTCCACAGAATGGAGCTGAAGCAACTTTT
AGTTATGTACTAAAAGGACCTGCCGGTCAAAAGACTATTAAAGTAGTTGCAACAAATACA
GTTCCAGAATCTGAATCTGAACCTGAACCTGAAACAGCAGAAGCTACGACAACTATCACA
GTTCAAAATAAGACACAGACTACTACAATAACAGTAACTCCTGATAGTCCTTCGGAAGGA
GTAATCGGAACAGCTGTTCAATTTACCGCTGCCTTAGCTTCTCAACCTGATGGAGCGTCT
GCTACGTATCAATGGCACGTCGACGGTTCTCAAATTGGTGGAGAAACTAACTCTACATTT
AACTATACTCCTGCCACAAGCGGAGTTAAAAAAATTAAGTGTGTAGCTCAAGTAACTGCT
GAAAATTACAATGAAAAGGAAGTTACTTCCAATGAAGTATCATTAACGGTTAATAAGAAG
ACAATGAATCCACAGGTTACATTGACTCCTCCTTCTATTAACGTTCAGCAAGATGCTTCG
GCTACATTTACTGCTAATGTCACTGATGCTCCAGAAGAAGCGCAAATTACTTATTCATGG
AAGAAAGATTCTTCTCCTGTAGAAGGGTCAACTAATGTATACACCGTTGATACCTCATCT
ATTGGAAGTCAAACTATTGAAGTTACTGCTGTCGTTACAGCAACCGACTATGATAGTAAA
ACAGTTAAAACAACAGGTCAAGTTCAGGTAACTGATAAAGTTGCTCCGGAACCAGAAGGT
GAATTGCCTTATGTTCATCCTCTTCCACACCGTAGCTCAGCTTACATCTGGTGCGGTTGG
TGGGTTATGGATGAAATTCAAAAAATGACTGAAGAAGGTAAAGATTGGAAAACTGAAGAC
CCAGATAGTAAATACTACCTGCATCGTTACACTCTTCAGAAGATGATGAAAGACTATCCA
GAAGTTGATGTCCAAGAATCACGTAATGGATACATCATTCATAAAACTGCTTTAGAAACT
GGTATCATCTATACCTATCCATAATCATAAGGGGCTTCGGCCCCTTTCTTCATTTTGAAA
GCACACAAAACACAATCAGAAAATGATGTATATAATGGCAACAACTCGATAACATGAGAT
TGATTATGAGAACTGAGGTTGTGGTGTTTACTCTTCATGAGTCTGGAAAGTCATTCATTG
AAATTGCTCGTGAATTAAACTTACAGGCAAAAGAAGTTGCTGTATTATGGGCTCGAGCTA
TGACTGCTAAGAATAAATTTGAAACTCGAGAAAAAGTTGTCTATAGAAAAAGACATATCA
ATAAAAAGGTGAAAAATGGAACAGTATGATCTTTATGAAAATGAATCTTTTGCTAATCAA
TTACGCGAAAAAGCATTAAAAAGTAAACAGTTTAAGCTAGAGTGTTTTATTAAAGATTTT
TCAGAACTTGCTAATAAAGCAGCTGAACAAGGTAAAACATATTTTAGTTATTATTGTACT
GCTCGCGATAAATTGATTACTGAAGAAATTGGTGATTGGCTGAGAAAAGAAGGATTCAGC
TTTAAAGTTAATAGTGATCAGCGTGATGGTGATTGGTTAGAAATTACATTTTGAGGATTA
ATTATGTTTAAAAAGTATAGCAGTCTTGAAAATCATTATAACTCTAAATTTATTGAAAAA
CTTTATAGCTTGGGATTGACTGGTGGGGAGTGGGTAGCTCGTGAAAAGATTCATGGTACA
AATTTCTCATTGATTATTGAGCGTGATAAAGTAACTTGCGCTAAACGTACTGGCCCTATT
CTTCCAGCAGAAGATTTTTATGGCTATGAAATAGTACTCAAAAAGTATGATAAAGCTATT
AAAGGCGTTCAAGAAGTCATGGACAGTATTTCAACTTCAGTGCCGGTGACATATCAAGTC
TTTGGTGAATTCGCTGGTGGTGGAATTCAGAAAGGTGTTGACTACGGTGAAAAAGATTTC
TATGTATTCGATATCATCATAAACACGGAAAGCGGTGATACTTATTACATGAGTGATTAC
GAAATGCAAGATTTCTGTAATACGTTTGGTTTTAAAATGGCACCAATGCTTGGACGTGGA
ACATTTGATGCTCTTATCATGATTCCAAACGACCTTGATTCCGTCTTAGCCGCTTATAAC
GTTACGGCATCGGAAGACCTTGTCGAAGCAAATAATTGTGTATTTGATGCTAATGTTATT
GGTGATAATACTGCAGAAGGTTATGTTCTGAAGCCTTGTTTCCCTAAATGGCTGCCAAAC
GGTGCTCGTGTAGCGATTAAATGCAAGAACTCTAAATTTAGTGAAAAGAAAAAGTCTGAT
AAGCCTATTAAAGCTAAAGTTGAGCTATCAGAAGCTGATAACAAATTGGTTGGAATTTTA
GCTTGTTACGTTACACTAAACCGCGTAAATAACGTTATTTCTAAAATTGGCGAAATTGGT
CCAAAGGATTTTGGAAAGGTGATGGGGCTAACTGTTCAAGATATTTTGGAAGAAACTTCT
CGTGAAGGTATTACTCTAACTCAAGCAGATAATCCTTCTTTAATTAAAAAGGAATTAGTT
AAAATGGTACAAGATGTACTTCGTCCAGCTTGGATTGAGTTGGTGAGCTAAATAAAAAGG
GACCTTTCGGTCCCTTTGTTTTATTCATCAATAATAATTTTTGGTAATTTAACACCTAAT
AAAACAGACAAATCTGAACGACCCGCCATTTTATCCATGTCTCCACCGTCAATTATTCTT
GCTTCTTTTTCATCTTTTGCTACGGTATAAGGATTTGCTGATAAAGCATATCTAACTAAT
AAACCGATAGACGGTTGTAAGCTTTCTGGATCAACAACAACTTTAAATGCACCTACATGT
TCAGGGTCATCTAAGTCAAGACCTTCTGTATACGGAGCATAGAAAATTGATCCAACAATT
TCTTTTTCACCGATATTTTCTACTACGCCAACGATTACATAATCTAATGGGCTGTTAGTA
TCGCAATAAAGCGGTAAACCATTAGCTAAGAACCCGTAGGCATTTTGTGAAAGATATTTG
TCATCTTCTGGTTTATGTTTTAACCAACCTGATGCAGCAAGAATCGCAGCGGCACGAGCT
GAAGCAACACAGAACGTTGCTGTATAAGTTGATTCTTTTTGGATATGTGAAACCATTTCA
CATACCATTCGATATAATGAACGACCAGCTTCAGGTGCAGATGCATAACTCAAATCGATG
AATCCACTATCAGTAATTCCTGTAACTTTATAGCGTTTTGACACTGTAATCAAAGACTGC
AGAATATCTTTATTGATTTCATCTGCCATTTCAGTTGCAAGCAAATCTTCCAAGAAATTA
GGAGCATCGAATCCATTTGCTTCTAAATCTTGCGCTAATTCAACTGTGATGCCAGTTTTA
AGTTTACGAGATTTAACCGCGGTTTGCCATTTATTAATCTGAAATCTAGCATCCGCAATT
TCGCTATCAGAGCTTTCAAATTTGCTTGTTGACGCTGCGTCAGAAAATAGACGAACCTTT
AAAAGAACAATTGCAATCTGAAGAGCTAACTCTAAGTCGCTTTCTTCAATATCAGCGAAT
GGAGTATCTTCTAATACTTTATAAACGATGTTATTATATTTGAATAAATCGCCTTTATTG
AGAGTTAATTTAGACTCTTCTGTTAATTCTGTGATTTGTTCTCGGTCTACATATCCAGCT
TCGCCAGCGTAAGTAGCACCAGTTTTAAATGTAAATTCATTGTCTGGGTTAAGGTATTTG
ATACCATAAAAAGCAGCAACAGGTTGATTAGTTCTTTGCGTTGCTACAATGTCAGAATAT
ATTAATTTAGTGGTAGCGCGAGTCAAAGCAACGAGATTTGGGCGACCGATTGAGTTGCTA
TTCGTTGTGGTTGATTCGCGCAGAAGTTCGTTGATTTTAGCCATTGCGCTTTCCTTTTGG
TTTATATGATTTATTTATACCATAAAAACAACTAAAGGGACCCGAAGGTCCCTTAAATCG
TCAAAGATTAGATACCTTTAACATATACACGTCTAAAGTAAGCGTTTTTACCAAGGCTAT
TCAGAATAGAAGGCATACCGCTCTGGATGCGAGAAGCCGGAGCCTGAGCAGCGGATTCTG
CAAATGGGTTGATACCGATACCGTAACGAGTTTTGAATCCCATTACCGGTTGGAAGTTCT
TCGGATCGGAACCACGCAGCGGAGTCAGAGCTACATATGGAGCATAGTAAATACCAGCAT
CCATTTCGTTCGGACCTTTATAACCTACAGTGAAATAATCCTGCTTAGCATACTGGTCGA
TATATACACGGTATTTACCACCCAGAACACCAGCAAATACTGACTTGGTAGTATCAGTGT
TAAAGCCGGTAGCCAGACCCTGTGCAGCATAAGAAATGCCGGTATCAACTGAAGCCAGAA
CGTTAACTACGTTACGGGAAGCGATAATGAAGTTACCTTCACCACGACCGGTCTGACGAG
CAATTTCAACTGCTTCTTTGTCAATCTGGAACAACAGAGCTTTAAAGGATTCACCTGCCC
AGCGAGCACCACGAATATCGATTGGGTCCTGGAAGTCAAATACACCAGCTTTAGAACCCG
GAGTCAGGGTCATACCAGATTTACCAACCTGAGCTGAGTAGTTAATCCAATCAACAACTT
CACGGTTGATTTCCAGCATAATTTCTGTAGCCAGAATACCAGACAGTTCAGCATCAGCAT
CCATACCGTGAACAGCGCGGAGGTCCTGTGCTAATTCGATAGAGTAAGCAGCTTTCAGCT
GACGAGATTTAGCTTCGATAACTTGCTTATCGATACGGAAGCCCATTTCATTCCATGGGT
TATCGGTAGAACCATTGAAACCTTCCTGAAGTTCAGCGATAGAAGTAGCCATACCTTCAG
CGATTTCTACCAGTGCACCAGCTTCCATTTGTTTCTTAATTTCAGCATCTAATTTAGCTG
CATCATCGGCACTTGAACTAATTGTTACTTGAGCAGAAGCTTGCAGATATACAGTACCAG
TTTCCTGGAAGAAGTGAGTATAGATATCACCTACTGTGGTTTGTGTGCTAACAGCCAGAG
CTGGGAATTTCTTAGCAGCACCCTGACCAGAGAACATTGCATCTGGACCATACATTGGGT
GGAATGCTTCTTTAGCGCCGGAAGCGATTGGGTCTTTACCATATACTGCGCGCAGTGCGA
ATACCTGGCCAGTCGGGCTGTTCATCGGCTGAACACCACAAATATCGAAAGCAATCAGGT
TAGGAATAGCACGACGTACCATACCCATAACAGCTGGGCCAATCTGAGTTACTGCGCCAG
AAGTCTGACCTGCAGCGATGTTAGTAGCATTGTAATCATGGTCACCACCGATTTCAGCTT
CTGTTAAGAAAGAACCGAATGCCTGAGCAATTTTTTCGTCTTTATATTCCGGAGCTGTCT
GGAAATCTTTTTCCTGGTTTTCAAAGATTTTAGCGATAATCGCTTGTTTGCTATTAGCAA
TTTCCGGTAAACCTTCACCTTCCAGTAATGGCTTCCATTTGTTCAAAAGTTCAGCTTTAG
TTTTGATAGTCATTTGTGTTAACCTTTAAAATTAGAAACGAGATGCGACTTTCGCATATA
CACTTACAATATCTTCTGCACCTTGTGCAGATTTATCTTCTACAGCTTCAGTGACGAAAT
TCAGTCCGGCTGCTTCAGTATCAGGAGTATTTATACTCTCAGTAATAGTGCTTTCATCTT
TATTAGATTTCTTCACCATTTCTACGATGGCACTCAATTTGCTTGAGAATGCATCTGAAT
AATCAATACCTTCGACTAGAGCAGAGACTTTTTCTTTTTGAGACTCAGTCAGATCTTTAG
TACTTTCGCTCAATGCCACTTCACGCTGCACATAATTGATATATGCGTCACGCTTATTGA
GTTCTTCGAACAGACGAGCTGATTCTTCTTTATGTTCTTGCAGTTCTTCTTCCATTTCAG
CTACAACATCAACTGATTCTTCTGGAACAACAACGTTGTGTTCAACGAAGAGCTCTTTCA
ATCCACCAAGCATGGATTCAAACAGTTCGGCTTTGATACCTTTATCTACTGCTAATTTAT
TTTCAGTGAGCCATTCTTTTGCAAGATGGTCAAGGAATTTAGAAGCTTGCTCAGCGATTT
TCTTCTCAGCTTTTTCTTCTGCTTCTTCTTTATTTTTTTCTACTTCTTCTTCTGCTTTTT
CAGCAATTTTAGCGATATGAGATTCAGCTAATTTAACGGCGTGCTGCTTGACGGTAGCTT
CGAATACAGTGCCGAAAGTTTCTTTTGCTTCCGGAGAAATATTAACTGATTCGAAAATAC
TATCAAGAGCAACGGAAGCATCAATTTTCTGCGCTTCGGCAATCAGTTGTTCTTTAAGCA
TTTTGTAGTCCTGTTGTTTAGATAATAATATTTATAACGCTTTTTTCATGGCCTCTGCGA
GAGCCATATAGGCGTCATCGGCACTTGTATCGGCTTCCGCCGTCTGTGATTCGGTAATTT
CCTTAGGAGTTACCCATGCATCTGGAGCACTTGGACCCCATACTGCATCAACACCTACAG
TTAATTTGAATCCTTCGTTTACGATACGATAACCTTTATTTGTGTCAGTCAATGAACCTA
ATCCACGAGAAGAAACTCCTGGAATCCATCCGGCACGAATATTAGCTGCTAATTTATCTC
CAGGACCGTGGTCACCTTCAATAACACGAGCTCGTCCGTATACGTCATTTCCTTTCCACC
ACATATCTTCTATAATGATAGCGGCTTGCATCGGGTCAACATTAGCGCGTGGAGGATGAT
TTAATTCTCCGAGAGCTTGTTTAGTTAAAACTTGCTCATTAATATAGTCTTTTACCGCTT
TTTCTAATATACGTTTTGGATAAAGACGTTTATTTCTATTGACGACTTCCGCTTGCATGA
ATATTCCTTCGATGTATAAACCCGGTTTTAAACCTAAGTCTTTTCCATCATGAGATTCAA
GCATTGGTACGCCATCAATAATTTCGCCAGGTTGACCCCAAGTTTCAATTAGTAATTGGG
GTTCATTCATTAGCTTAATCCTAATGCTTTACGGCGTTTAAGAGCTTTTTTACGCTTACG
CTGAGCACGAGATTGACCTGCTGGATTGGCAATCTTCGTTTTGGTAGCTTTACGAGCAAT
TTGTCTACGTTTTGCTTTAGACAACCCGGTAGTTTGAAATGCATTACGTTCGCGGGTTTT
GCGATCTTTAGTGCGAGTAATTTCACCACGGGCAGAAACATGTTTAACGATAAATTCATT
TAACGGCATATTTTCATTAATAGAAGCTAATGCAACCGCTAAATCAGTTTCATCATCAAG
CATATTCTCGACAATTGTATTTATATCGTCTTTATTTAAAGCAGAAGACAATTCGTCAAA
GCGACCCTGTGCTTCAGGAATAAGTGCTTCGACATTCTCGAGAACTAATTCATGAGTTTC
AGGGATCAGAAGCATTATTCATCATCCTCGTCTTCGTCAGAGTCTTTGTCTTTTTTGTCG
TCTTTATCATCACTATCTTCGTCTTCGTCATCATCCTCGTCATCAGGTTCTTCACCTTCG
ATTAAGAAATTACGAGCGATAGCGATTTTTTCTTCTTTAATTAAATCAGTCGTTCTTGCA
GCCATGGCTTCAGCAAATAATTTACGAGCGGCTACGAGGTCGTTTGATTTAATAGCTTCA
ATTAAACCTTCCATTAAAAATCCTCTTGTTCTTGGTCGGGGTCTTGGAAACGAGCCTCTT
TAGACTCTTCTTCAATTTGCTTGGCTTCTTGTTCTATTTCTTCATCAGTCATCTGCAAAA
TATCTTTCATAGCAGTTCTGTGAGAAATATATTTACCAATAAATGGTTCTGCCATGGTTA
GCATATTAATTCTTCGTTCCAAAATTTCTGCTTCTTTGAGCTCAGCAAAGTAGCTATCCC
GATGAAATTCTATCTTAATATTATTTATTTCATCATTCCACTCATCTTCTGTGATTATAC
CTTTAAGCAAAAGATTTGTTTTAAGTGGATCTAGGAAAACTTCTTCAAACTTGTGCTGTA
ACTCACGAATAAATTTAGCAAACGTTAATTCATCACGTGTAATGCTAGTTCCAGAATCAA
ACATCACACCGCCTTGTTGGTCTTGTGGAATGCGTGAAAGAGGAACACGTAATGCCATGT
AAAGAGCTTGTCTAAACCAACGAATATCTTCCATATTGCCAGTATTATCAGCGCCAGGAA
GAGTATCAACTTCTGTCACAGCTTTACCATCACGGCGCTGCAACCAATAGTCTTCGGTCA
TAGACATATTATGTTGTTGATTTTTTATTTTACCTGTTGATGCATCATATACTACACGGT
TTTTCATCGTGTTCATAACATGTTGCATGTGCTCAGCAGCTTTACGAGCAGGCATATTAC
CTGTGTCTACATACCAAACACGACGGTCAGGAGCACGAGTAATGCGATAAATGACTACAG
CATCTTCTAATAATTTTAATTGGTTAGCAGGTTTAACAGCACGATGTAAATACCCGATGA
TATTTTTGCCACAGCAATCGACTAATCCAGAATGGGCATAAACAACAGCAGCTTTTGGAA
TTTTTATTTTTGTGCCAGCTTCATACATTCTACCATCACATGCATATGACTCATGGGCAG
TATCATATATAAAATATTCTTTGTAACCTTTAACTATTTTTGTGCCAGCTTCAGTTTCTG
TTATAATTTCGCGAACATACTGAACTTGGCGAGGGTCTAATCTACGTAATTCTTTTATGC
CTTCTTTTGGACGTTTTGGATCAATGATTTTATGAAAGAAAATTCTTGAATCAACATACC
AACGTCTAAAATGATCAGAACCTTTTCGTTGAAACGATAGATGATTTAATACATCACTAA
ATTCATCTAACATCATATTTTTAATTTTTGGACTAAATTTAGATTTATCCAAATTTAACG
CTACGACTTCAGTATCATCTTCGTAGACGATAGCATCTGAAACGATTTCTGAAACTGCAT
TATCTACTTCATAGTTATTCATGAGATTACGATATGTATCAATAAGCTCACGAGTAGTTT
TCATTCCTGGTTCATATGAACCAAAAATTGTTTGGAATGCAGCATTATAAGGAGAAGCAG
CTTCATTCGAGCTTACTTCAAATTCTCTTGCTCCATCATCAAGCTTTGGGGCTGTAATGG
AAACAAGATCTTCTTTTTCTTGGTCTTTAAAATTTCGTTCGTCCATTTTAGCCCATGGAG
CAAACAAACTTAATACATTAAATTTCATTGTATTCTCCAAATGGGAATTATAGTTATATT
TATAATGGACTTCTCTGCTTTAAGCAGGATGGGGATTTCTCCCCATTCATTTTATTCCCA
ATAATCGAGAGCAAGAGTTACTTCAAAGGTTTGGATTTCATTGTTTGAATCCCAATCTAA
TTGAAGTTCACCCACGTTAGTAGGCCACAGACCTTTAATTTCAATTTCTTTTGTTACTGT
TTTAGCATCACGAGCATATTGACGAACAATAGCGCTCTTTTTATACTCTGCAGGTTTTCC
ACCAGTAATTTCGTTTCCTTGACCAGCAGCAATGCTTTGCCAATCAACAAACTTCTGGCG
AGCATCATGAGCTTCATCGTTCATTACTGTAACAGTCCAGTCATCGAATGTACGATCGCC
TGCTACGTTAATTTTACGGTTCATAAATCCGACTGGAATTTTTTCTACAATACCAGCTGG
TAAAGCAGTGGCTTTACATTGGAATGTAAAATTTTGTCCAAGATAAGAAATTTCTACTTG
GAATAAGTTAGGTCGAGCAAAATCACCAGATTCAAACGCTCGTGTTACATCATCTACAAA
CATATTAGCCTCTGTAGTATTTATATCCCTATGTTTAACCTAGGGCATATAGAATTAAAG
AATTAAGGATATAGTGTATTTATATGGCCTGCCGAAACAGGCCTTTAGAATGCACCGTAT
TAACCAGCAAGACCAGTTAACTCATCGAAATCTGCACCAGTAGCAGTTGCTACGAAGTTC
AAGGTAATGTAGTTAATGCTTCTAGCCGGTTGAATGTAGAATGTTGCAACAAACTCATTT
CTATCAATTACTGACGGAGTGTTATTTGTTGTATCGCAAACTACACGATATTCATAAATT
CCACCGAGAGCTTTAATTCCCTGTAAGTACTGGGCAGTTTCTGTGCGGAATGATGAACGA
GTAAACGCGTTGTTTAATTCGAACAAACGATATTTTGAACTACGTCCGATGTTCGTTTTC
AACATATTAAACAGACGACGAACGTTAATACGATCAAATGGAGAAGGAACAGAAGTAGCT
GTTTTATCACCATATAATACGTAACCATCACCACCTGTACCAGTCACTGGGTTGATAGCT
TCTTGGTATAAACGGTCGCGCTGAGCTTGGCGAGTTTCAATAGCAAGTTTAATAACGTTA
AGAATCTGGCCGCGATTATAACCAGCTGGAGACATCCAAGTCTGAGATACGTTATCAGTT
CTTGCGCATAAACCGGCAATATCAGCTGCTAATGGAACCCAACGATTCACATCATTATAT
TTGTCATACTGATATTTGTAGTTACCATCAATTGCTGCGTAGGTTGAACTGATATTAAAG
TTATTATCAGTGTATGAACCTGCTGCAGTTCTCCAGTTAACTAAATTATCTACTGCACGA
GTTACAGGAATTCCAACTACAGTTTCACGCGGAGGTGAGCACAATACTAAGCAATCTTGA
CGAGCATCACCAATTGAAACAACATGTTTTTGGACAGTAGATGCTGTTTCAAGAGATTCA
CCGGCACAAGAACCCGCAATAAACAACTGAACGTCAACAGATTCGCGGTCAGCAAAGAAG
TCCCAAGCTTCCATCAAATCTCCTGCTGTTACTTCAGCATTTGATGATAATCCACCAGAC
AGAGTTAAAATTCCAGAGAAGCCTTCTGGCCAGTTTTGCGCAGTTGCGAAAATATATTCT
GAACCACCTTTTGCGAAAAAGTCATCAATATAGATGTTACTATCGTAAATATCTTTTTCA
CCACGCTTAGTTGAAAGAACAATGCTTTGAACAATAGCATCATTGCGACGAACTATAATA
GCGTACTGTGAATCAGTTTGCGGTCCATATCCAAACACTGCTTTGGCAGTAGATGCACGA
GTACCACCACCTGGATAAATTGGGAGTAATGCAGAAGCTCCTTTTGCATAGTCAGCTTTA
GATACGATTTCAATTTCAATTTTATCGCCTAATTCGCCTGGATAAAGAGCTACTACTCCT
GGAATTCCGTATTTTTTAAGATTTGCTTGAAAGTCAACCGCTGTCATAGCAGCTTCAGCA
TTTTCAATTTCAGCTAATAAAATACCAGAATCAGTAATAATTTTTCCAAGAGTTATTACT
GCAGCTAAACCAGAGGAAGATGAAGAAATTTCCGCAGTCCAGTTAGAACCTAATTCCGGG
TATTCACCGACTTCTTTTGCTTTAGCGATAATTTTTGCAGTAGGAATATTAATTTTCTTA
ATTTTTCCATCTGCGTCTACTTCAGTAATTTTACCTTCAGTTTCAATATCATCTGAAACA
TATTTGACCGTGATTTTATCTCCAACCGCATAGTTACTACCTGGGGTAGAAATTGTGTAT
TCAATATTACCAGCAATTGGCGATGAGTTTTTAGCGGTATCTCTATCAACAGCACGAACT
ACTCGTAAGTCATTTCCGTACTGCAAGAAATTCATCGCAGACATAAAATAGTCAGCAGTT
TCAGCGGTTGGTTGACCAAAAGTATTAACTAAATCTACTTCGTTTGTAACCTGTTTAATC
TGAAAAGCAGGACCCCACTGGAATTTACCGGCCAAAGCTGCTGTACCAGTAGAGTTATTA
ACCACGGTGCTTTGAACCGTAGTTTCTTTGAGCTCAATGCCCGGAGATAATAAAGTCATT
TTTAATCCTCTTTAATATGCTTTAATATATTTATACCATTGACATACCATGAGATACTGG
AACATACTCAGCAGAATGAACCGAATCAACAAATATAACTGGAGCGTATTCATCCCCCAT
ATCTTGAAGCTCTTTTGAAAACACTTCAGATGCTAATCGCATGTCATCTTTGTCGGCATA
ATCAATAAATTTTGATTGTGTTGATAACCATCCAAAAATTACTAAAGACATTACTAAATC
GTCATGATAACCTTCTTCAGCCGCCCAAGACACGCCTTTTTCACTAAACGTTCTAAATTC
TTGAATAGTTGCACGGTGATGAATAATAAGCTTATCTTTTTCAATAAGGTCTTTTAATGT
AGAGCATCCGACTGCTTTCGTTCGTTTAGTTTGCTTCATTCCTAAATCAGTATATGAATC
GCAAATAACGCCTTCGTATTCTAAATCCATATAAAGTGATTTAGCAACTGACACACCAGT
ACTATTTAATTCAATATAAACTGGGCATTCATTATATTCTACTAAATAACGCATAACGAT
GTCAGGTAGAATTAAATGAGAAATAGTGTTTGAGTGTAAAACACCAACTTGTTCCCACAC
ATCATCGGTAACATCAATAATATGTAAAGCGTGGTAATCTTGCCCACGACCTTCTGAGCA
GTCTAGAGTTGCAATATATTTTCTATCTGGTTCAGGGCTTTTAAATCGATGAAAACCGTG
ATCATCTGGAGTTACTTCAATGAAATCCATAACAGCTAATTTCATTCCTGAAATTAATGT
ACCGGAAGTCCCTTCAAATGCTGCGGTGTGTTCTTGACGAAATTGAGCTAAAGTAGAACC
ATTAATGGTTTGTATACTCCATTGCCATCCATCGTCAAAAATATCTTCATCATTATAAAG
ACGTTCTTTAACTGAATTCCAAATAGCAGTGTATGGTTCAAATCCTGATTTACCTTCAAC
AGCAGCAGTCCAAATATCATAAAAATGATTTAATCCATTAGGAGTCGTAGTAATAATAAT
TTTTGAACGACGACCAGATGAAATAACTGGTTGAATAGCAAGCCAGGAATCATGGAAGTT
TGGAATAAATGCGCATTCGTCAATATAAATCATTGCGAACGAGTTACCACGAACTGCGTC
TGGAGAGGAAGCATAGGCTCCGATTGAGGAACCATTATCTAGTTCAATTGAACCCTTATT
CCATTCAACTATACCAGGCTGCAAAAAGTCAGGAAGCAGTTCAATTGCTTGCTTAGTACG
GTCTAAAACTTCCGCGGACATTGAGCCTTTATGCGCAAGAATACCTACAGCTTTATCCTT
GTTAAAACATACAAAGTGTGCAAGGAAAATAGCTACTACCGTTGTTTTACCGAGCTGGCG
TGATAGATTACAAACAGTCATACGTTTAGATGACATTATTTTGAGCATATCACGCTGATA
GTCACGTAATTGAACCTTTATGACACCATAGTCGATATGAGTAATGGCGCAGTATGTTTC
TGCAAAATATACAATATCATCTCGGCATTTTTTCCACTCCTCAACCATTTCACGAGTCCA
TTGTGTTTTAATATTAGCTCGTTTCAAGTTAGGAAGACCCATATACCGAGATCTTTTATT
ATTCTTATCTTTATAAGTTTGAAATAATTCAGGCTTATCAGAGTTGTTTGGAATTTTTAC
TATTTTATGTAGACGAAGGTAATCACTGAATTTTTCAGGATACCATTTTCCATCCCACTG
AGATTTTATCCAATGAATTCCATCTTCATCTTTTCTTTCCGCTAAGCTTGGGTGTTTTAT
TAAAATTTTTCCAGCTTCATTTAACGGATGGAAATCATTTAATGCATTAATCGGTTGTTC
CATTTATCACCTTCTCACGAGCTTCTTGAGCCTCGTAAGCATCACCAATTTCGTCCATTA
ATTCTGTTGGTGAACCCATGAATACTGTCGCATTCTGAATATTCATTTGACCTGTAGGAA
CAGCACCTTTAGTGCCAACCTGCTCAGATGTAATGTCTTTCATATCTTTATGAAGCTTCA
GTATTTCTCTGTTCGTCGTAGTCATTTGCCCCATAAGAGTTGCAAATACTTCCATGTGAC
GAGGAGAATCAGCATTCTTTGCTGTCTCAAGAAAAATCTTGGCTGCATCCATTAGCATTT
GTTGCTGAAAATGCATATTTCGACGAACTACTCCATAATCATCTTCTAAGTCAGGAGTGC
GGTTTTGTGGATTGCTTTTAACTTCTACTAATTGCAGAGGTTCATATACTTTAATTTCCT
CCCCGTCAATTCCGGGGAGATCAGAAATATCTAAAAGTTTGTTTATATCAAGACCTTCCA
TAATAACCTCTATGTTCTTGGGCCAGGAGGTTCTGGCGGTGTTGGTCTATTTACATTACT
AGTGAAAGTTTGTTTTACTGTTCCATCCCAGTCTTCTGGGTTAATATCTCGAGGAACAAC
TTCGCTATCGACAGATTCAAAAACACCTTCGCCATCAGGCAAATCTCTTGTATTGGCGTG
AAAATCTGTATAAGTAGTACGAATTAATCCTTCTGCATCATCTACTGGAGGATACATCCA
TCCATTTACTTCAAATGTTAATGACCATTCAATTCTACGACGAGATAAATTATCCCCATC
TATAGCTTCGTCTATAGCAGCAGACATTAATACAATTTTGATATCCCTTTTAAATGGAAT
ATCATTTCCAAACTGCTCGTACATAGTTGTATTAAAATGAGGTTGAAAATATGGAAGAAT
TTGTTCAACTATTTGAAACATATCATCTTCGTAGCGAGTAAAGATACTCAATTCATAAAT
CATTTTAATAGGAGATGGATTATACTGCGATACTACAGAAGTTGCACCTTTTTGCAGTAA
ATTCTGATTTAAAATGTTTGTTTTAAATGGAGCATTATAACTAAAATCAACTAAATGTAA
ATTTATACGAGGTAGAATGGTTTCAACCTTGGCCACATCTTCTTGTGAATTTATCGATGT
CCATTTATTCAATTTCATCATAAAGTGTTCCTTTGACGCATATGTAATAGGAACACGTAT
AAACTTATCACCAGATTCTAACTGACGTTTGATTTGGATATTTGAAAACAAATCGCCCAT
CAAGGTAGCATATCGTCTAAAAGACGAATTATAAAAATAACCAAACATGATTTCTCCTAA
TCTGGGCTTTAATTAGTTTATAATATTTATTAATCCATGAAATCATTATCAAATGGGCTA
GATTCGAAAGATTTGCCTCTATTATTGACAACAACATAAGGTTCAACATATTCTTTAGCT
TCAGAATTAATTTGATCTACTTCAGCATACTGATCAATATTAATATCATGAATACCGTTA
AGATTGCGAACAGGATTTAGTTCTAATTCACTAAATTCTGGAATGTTAATTCCTTCATTT
TTCTGTAGAACTGGATTAATTTCTTCTCCGGAATAAATGAATTTACCTGCCGTAATTTTA
CGAATAGCGTTTTGGCCTAATTGATAAAATGGATCATATGGTTCAACCCAGTTAATTTCA
AATAAGCTGTTATCCATAGGAAAATATATCAAATCGCCTTCTTTCGGTTCTTTTCCATTT
ACTTGGTGTTTAAACAAGTTTGGATTAATGGACAAAGTAACTTCATCCTGTACTTGCATA
CCAAAGTTACCAAAGAACGATTTAGCTCCTTCATATCCTTCAAATGAATTTAAATATGCA
GCAAATTTCCAAGCTTTAGTAAATTTATTTTTTAAGTCTTCGCCGAATACCAAATCAGGG
GAAACATACTCTCTTGGAACATAATAGCATTCTACACCTCGCATTTGAATGCTTTCAGCT
ACTAATACATCAGCTAATATTTGGCTGTTTTTATAATGATTGAAATTTACATAAGGATTT
AATATTTCAGTTTCATTGGTCTGAGAATAACCCGTGCGGTTTTCCAATTTAGCAAAAAGA
TTTTTATCGTAAGTAGCCATATTAACCTACCAAAATTCCAAATGGAGGGTCAAGTAAGTA
TAATTCTTCACGTAATGTTTCTTTTTCTAATCGAGCTTCTTCTATTAAACGCTGCCCATC
GATTGTAACACCGCCAGGAAGCATCATACCTTGGTGGCGTGCTAAAATTTGACCATTCAA
TTCTTTAGCTAAAGCTGTAGCATAGTCTTTCACCCAACGATTATTGTAAGCACCTTGCTT
AACATTTGGGTCTTGACCAACAACACGACCTACTAAATTGTGGTCTGGATTATTATATCG
TTCAGATAATGACCAGCTATCTTGTGGACCGACTGTTCCATATCCTACTGTATTTCCAAC
CATTTTATTAGTATCAATGTATGATTTAGTCCAGCTTTCTACGATAATTAAATCATATTT
TTGGAAGTTTCCCATGACTTTGAGTTGTTCATTTGCTGAATTAAACCAAAAGTCTGGAAT
AGGAGAGAGCATATCTTGCATCATTCCCATATAACTGGTAAGTTGAGTAAAATATCCTAA
ATCAGCTCCAAAGGCATTTGGTCCATAAAATCTATTACAAGACGTTCCCATTCCGCCATT
AATACCAGCCATTCCTAAAAGAAAGTCAGTAAACCACGGATATGTAGCATTTCCATCCAT
AGATGTTATTGACCCAATATTTGTGCGTAAAATGCGAGTTACTGCAAATACGTTGGAACC
TCTTAAATCGAAGACTCCGGTTTTGTACTTTTCTTCGTCATCCCCTACATAAAAAACATG
AAAACCTTTATTGAGTCCATCAAAATGGTATTCACCATATAATTCTAGGGCACGCTGGAT
GCAATCGTAGATTTGATCGGGTGTTAACTCAACATTAATAATCGGAGCCCCTAAACGTCT
TAGAATGACATCTTTGAGTTCCTTTGGATTCTGAGAATTATATCCTGACATTTAAAATCC
TTTGGGGCCTTGCGGCCCCATGTTATGCTGGTGATAAAAAGGTAGAAAGCAATACCCATT
CGCCGTCTTTACGAACGTATGCTTGCCCATCTCTTGGAGCTTCAGGAATATAACCCGCTT
CTTGTAGAGATTGAACGCTGCTCTGTAAAGAGGATATATCGTCTTTAGCTGTATTTACTT
CCTGCGTGACTGCCGCAATATTAGTTTCATTCGTTTTTATAGAATTAGTTAATCCTCGTT
CTTCAACAGTTGATCCGTCAGGATTATTTCCATTTATAAGTGAAGTAAGTTCAATAACTT
GACCTTTAATTCCTGTTCTATTATTTCCTATTTCAACTTGTATATTTTGAACATTATTGT
TTAAAACTGAAACTTCTCCTTCAAGCACTGAAACTTTATTTAATAGAGATCCAGAAGGAG
ACGGTTGTCCTCCGCTAGAGTCAGTTCCAACAATTTGATTTAACCACGAAACATTCGCTC
TTAAACCAGACGAAGTGTTTTCACCAACAATTCCGTTTAAAGACTCAATAGAAGTTGTGT
GATTTTTAATTTTTCCTTTAATACTAGAAGGAATATCATCTGAACCAATAGAAGTTTCAA
TAGATGTTAAGCGTTGTTTAACACCTTCACTCTGATTTAATTCATTATTAATAGCTTCAA
TGCTACTTTTGTTATTATTAATTTCTGTTATTATAGAAACATTTTCTGGGTATCCAATAG
AAGATTTAATTTCTGCTATATCAGAATTTATTAAAGTTTGTTTATCATCTATAGTGTTTA
ATCTATTATAAATATTTGGTCCAAAATCTACTGGTTTATTACCAAGTTCATCGCGTAATT
TACCAACTTCAACCGTCAATGATCCTACGTCAGATTCAGTAAATTTATTTTCTAATTCAC
TTAAACGAATGCCTTGTGAAGATAATAATGTACTATTTGTTATTATTCTATGTTTCATTC
CTGTGCTAGCATTACCAACAACAGGAAGACCGTTAATGTCTTGGCCAGAATATTGCCCTA
GTTCTTGTTTAATCCACAATAAATCATTTCTAATTGTTCTATAAACAGAGTTTTCTTCAT
CATTAAATGGACCTATATCAGCAATAATTTTGTCAACTGTATTATTAGTTCCAGTTAATA
TATCAGTGTGTTCATTAGTAAGTGTCTTTAAATTTGATATGTCATTTTTATTAACACTGA
TCTGCGATAGTGCTTCTATATCTCCTGATACATCTAAAACTCCTTGAATTGTTTTAATAT
CTTTCTGCGATGTTTCAATGGCAGTTTTAAGAATTCCCACATTTTCATCAAGAACCTCGA
CGTTTTTAAACACGGAAACAATTGGTCTATTCATTGATCCATCGTTTCCATACTTAGTGC
TAGCTCCTAGTATTTCTTCACCATTTTTAATCCATGAAATACGTTCCTGACCTTCGTCAG
GAACGCTATCAACAAAAGGTAAATCTTTTAATTCAATCATTTGTAATCCTTAGTAATGAA
CTTTAATAATATAGTTTAATGATATATTCCATGGTCTAGTTTCATAACCAATTAATCCTT
CACGATTAAGTGTACCAAGGGCATCTCTCGGTCCGCCTAATTCAAATCCATCATTAGTGA
AGTATGAAGCATTATCCCAGTCGGAATATTTTCTAGTTCCAAGATATCCTTGATAAACAG
ACGCGCCAAATGGGCCTTCACTTCTGTTATATTCCCCCCAACCACCGGCGTGTTTATGGT
ATGACATTTGTTGTGCTTGAACACCACCAACATGCATTCCGTCACATCCTACGCCAAGTC
TATCCTTTCCATAACCATCTTGTCCTCGTTGATTTAAAATATGACCGCCTGTGCCGGCTC
CTCTGACGAAAAGACCTCGCATATCAGGAACGCCAGGATTATTCCAATCGCCACCAAATC
TTGTTCCAACTACGTTTCTATAATCAGGAAATTGGTCACCTGACACGGTTCCACCATGAC
ACATAATCCAACCTGGAGGTGCTGAATCACCCGCAAACATCATAATAGTTCCAACCGGAA
CTCGTTCGGACGCATATCTTTCAGTGGCAACAGGTTGATCGCCATTTTTCCACATTCCAC
CGCGTGACCAAATTCCATTTGCTGTAAGATGGTCTAAATTTAAAGAACCATTAATAGTTT
GTCCACCACGAGTATTAATTACATCTGCGTTCCATGCTAATGCTCCAGAGCTATCACCTT
CAGGGGCAATACCGGCTTGCGTCGTTAATTTAACTACTCCACGCATAGAACCGGTAGCTC
CTCGACCATTTAGTGTTTCACCTGTTACTGCAACACCACCCAAATTATTATTAATTTCAG
TCTGCGTTCCTAGGCGTATAACACCCTTATATTCATGTGTTGCAACAGAATTCATAAAGG
TATACGGGGAAATTGCATATCCTTCGCGGAGAGTTCCTTGACGAATTTGTGCAACAGTAG
ATAATTGAACTACTCCTCTCACCGATTCTGTTGCAGTGTCTTCTGAAGGAGCTATTTGCG
AAATTAATTTTATTGCAAGTTTTTGGGTTTTTAGCGGAGTCATCGCAGTAGTATCATCAA
CTCCAGCCAAAGCTGCAGGTGTAGATGATATTTTAATAACCCCGTTTGATGACTCAGACG
AATATCTATTTTTGAACACATCGTCAGTGTGGTATTTCAGTTTTTGTGGTGTTATTGATG
AATTATTATCTGAGCCTTTTAATGTTTCTTCATTTGTTGAATAACGCGTTAAACCATATT
TAGTTTCAGTAGCATTTGGATACAATAATCTTGTTGCTAATGTAGCTGGTGTAACGATTT
TAGAATTATTAGTTCCATCTAAAACCTCTTGTTCTGACGCAATCATTGCTATTCCTTTAA
CTTCCATAGTTGCATCAGGAATACCATTTACGCCGATATTACTTATTTTTGATAATGCAG
ACTGTACTGTCGTAACAGTGCCAGGAAAATTCGATCCAGTAGGATCGAATTTAACATATT
TTGATTCATTTGATACGTGCTGATATGTATTGTTACTCATGCTACTCTCTCAAAATAATA
AAATACTGTAGGTTGTGTGTGACTATCTAGCGTAACAGTTTGCGCACCTAATAATTGCCA
AGTTCCATACCCAGGTTTAGAAGGAGAAATTATTTCCTGTTTGAAAGTAATTCCTTTATC
AGAGTATTGTTCTAAGATATGTTCTTGGTTATCTAAATACTTAACTTGTATTTTATTTCC
TATGGTAGGATGGTCTTCAAATGAATCAATTGCAATAAATTTTACAACCATTTCCTGTAG
AGCTATTCTAACGGCACTAGTAAATTCAATTGAAGTCATTCCATTAGTAGCTTTAACTGG
AATACCAAACATGTTAACAATAACTGGGTCACCTGATTTGACTGAAGTATCAGTAACAGT
TCCTTCAAATGACCAATAGTCGGTCTGTTGCACGCCAGTAGGAGATACGCCGCTTATATT
AACTAATACAGCTCCAACTGGTTGCTCTGCCATATTTCTAACGTCATTGATAGCAGATTC
TACATTTGAATCATAAAAACCTTTTGATATTTGTGAAATTGTTACAGCGCCTACTGGCTG
ATTATTCATAACCGAAATATCATTTTTCTTAGTTCTAAAACCAAGAAAATCTGCTAAGCG
GGAAATAACTCCCGCTTTATTATTAAGTAAACTCATTATGCAATCCTTATCCAACGGTAT
ACAGTAATAGAC

In another aspect, the invention provides an SF37B bacteriophage targeting a Shigella spp. bacteria. In certain embodiments, SF37B binds a molecule of OmpC. In certain embodiments, the SF37B bacteriophage has a genome comprising a nucleic acid sequence comprising SEQ ID NO: 9.

SF37B
(SEQ ID NO: 9)
TAAATCAGAATTTACTGCAAAAAGGTGCAACTTCTGTAGTATCGCAGTATAATCCATCTC
CTATTAAAATGATTTATGAATTGAGTATCTTTACTCGCTACGAAGATGATATGTTTCAAA
TAGTTGAGCAGATTCTTCCATATTTTCAACCTCATTTTAATACAACTATGTACGAGCAGT
TTGGAAATGATATTCCATTTAAAAGGGATATTAAAATTGTACTGATGTCTGCTGCTATAG
ACGAAGCTATAGATGGGGACAATTTATCTCGTCGTAGAATTGAATGGTCATTAACATTTG
AAGTAAATGGATGGATGTATCCTCCAGTAGATGATGCAGAAGGATTAATTCGTACTACTT
ATACAGATTTTCATGCTAATACCAGAGATTTGCCTGATGGCGAAGGCGTTTTTGAATCTG
TTGATAATGAAGTTGTTCCTCGAGATATTGACCCAGAAGACTGGGATGGAACAGTAAAAC
AAACTTTCACTAGTAATGTAAATAGACCAACACCGCCAGAACCTCCTGGCCCAAGAACAT
AGAGGTTATTATGGAAGGTCTTGATATAAACAAACTTTTAGATATTTCTGACCTCCCCGG
AATTGACGGGGAGGAAATTAAAGTATATGAACCTCTGCAATTAGTAGAAGTTAAAAGCAA
TCCACAAAACCGTACTCCAGATTTAGAAGATGATTATGGAGTAGTTCGTCGAAATATGCA
TTTTCAACAACAAATGCTAATGGACGCGGCCAAGATTTTTCTTGAGACAGCAAAGAATGC
TGATTCTCCTCGTCACATGGAAGTATTTGCAACTCTTATGGGGCAAATGACTACGACGAA
CAGAGAAATACTGAAGCTTCATAAAGATATGAAAGATATTACATCTGAGCAGGTTGGCAC
TAAAGGTGCTGTTCCTACCGGTCAAATGAATATTCAGAATGCGACAGTATTCATGGGTTC
ACCGACAGAATTAATGGACGAAATTGGTGATGCTTACGAGGCTCAAGAAGCTCGTGAGAA
GGTGATAAATGGAACAACCGATTAATGCATTAAATGATTTCCATCCATTAAATGAAGCTG
GAAAAATTTTAATAAAACACCCAAGCTTAGCAGAAAGAAAAGATGAAGATGGAATTCATT
GGATAAAATCTCAGTGGGATGGAAAATGGTATCCTGAAAAATTCAGTGATTACCTTCGTC
TACATAAAATAGTAAAAATTCCAAACAACTCTGATAAGCCTGAATTATTTCAAACTTATA
AAGATAAGAATAATAAAAGATCTCGGTATATGGGTCTTCCTAACTTGAAACGAGCTAATA
TTAAAACACAATGGACTCGTGAAATGGTTGAGGAATGGAAAAAATGCCGAGATGATATTG
TTTATTTTGCAGAAACATACTGTGCCATTACTCATATTGACTATGGTGTCATAAAAGTTC
AATTACGTGATTATCAGCGTGATATGCTCAAAATAATGTCATCTAAACGTATGACTGTTT
GTAATCTATCGCGCCAGCTCGGTAAAACAACTGTAGTAGCTATTTTCCTTGCACACTTTG
TATGTTTTAACAAGGATAAAGCTGTAGGTATTCTTGCGCACAAAGGCTCAATGTCTGCGG
AAGTTTTAGACCGTACTAAGCAAGCAATTGAATTGCTTCCTGACTTTTTACAACCAGGAA
TTGTTGAATGGAATAAGGGGTCAATTGAACTAGATAATGGTTCTTCAATTGGCGCTTATG
CTTCCTCTCCTGACGCAGTTCGTGGTAACTCGTTCGCAATGATTTACATTGACGAATGCG
CATTTATTCCAAACTTCCATGATTCCTGGCTTGCTATTCAGCCAGTAATTTCATCTGGTC
GTCGTTCGAAAATTATTATTACTACGACTCCTAATGGATTAAATCATTTTTATGATATTT
GGACTGCAGCCGTTGAAGGTAAATCTGGATTTGAACCATATACTGCTATTTGGAATTCAG
TTAAAGAACGTCTTTATAATGATGAAGATATTTTTGACGATGGATGGCAATGGAGCATAC
AAACCATTAATGGTTCTACTTTAGCTCAATTTCGTCAAGAACACACTGCAGCGTTTGAAG
GGACTTCTGGTACATTAATTTCAGGAATGAAATTAGCTATTATGGATTTCATTGAAGTAA
CTCCAGATGATCATGGTTTTCATCGATTTAAAAGCCCTGAACCAGATAGAAAATATATCG
CAACTCTAGACTGCTCAGAAGGTCGTGGGCAAGATTACCACGCTTTACATATTATTGATG
TTACCGATGATGTGTGGGAGCAGGTTGGTGTTTTACACTCAAACACTATTTCTCATTTAA
TTCTACCTGACATCGTTATGCGTTATTTAGTAGAATACAATGAATGCCCAGTTTATATTG
AATTAAATAGTACTGGTGTGTCAGTTGCGAAATCACTTTATATGGATTTAGAATACGAAG
GTGTTATCTGCGATTCATATACTGATTTAGGAATGAAGCAAACTAAACGAACGAAAGCAG
TAGGATGCTCTACATTAAAAGACCTTATTGAAAAAGATAAGCTTATTATTCATCACCGTG
CAACTATTCAAGAGTTTAGAACGTTTAGTGAAAAAGGCGTGTCTTGGGCGGCTGAAGAAG
GTTATCACGACGATTTAGTAATGTCTTTAGTAATTTTTGGATGGTTATCAACACAATCAA
AATTTATTGATTATGCTGACAAAGATGACATGCGACTAGCATCTGAAGTATTTTCAAAAG
AGCTTCAAGATATGGGTGATGAATACGCTCCAGTCATATTTGTTGATTCGGTTCATTCTG
CTGAGTATGTTCCAGTATCTCAAGGTATGTCAATGGTATAAATATATTAAAGCATATTAA
AGAGGATTAAAAATGACTTTATTATCTCCGGGCATTGAGCTCAAAGAAACTACGGTTCAA
AGCACCGTGGTTAATAACTCTACTGGTACAGCAGCTTTGGCCGGTAAATTCCAGTGGGGT
CCTGCTTTTCAGATTAAACAGGTTACAAATGAAGTAGATTTAGTTAATACTTTTGGTCAA
CCAACCGCTGAAACTGCTGACTATTTTATGTCTGCGATGAATTTCTTGCAGTACGGAAAT
GACTTACGAGTAGTTCGTGCTGTTGATAGAGATACCGCTAAAAACTCATCTCCGATTGCT
GGTAATATTGAATACACAATTTCTACCCCAGGTAGTAACTATGCAGTTGGAGATAAAATC
ACGGTCAAATATGTTTCAGATGATATTGAAACTGAAGGTAAAATTACTGAAGTAGACGCA
GATGGAAAAATTAAGAAAATTAATATTCCTACTGCAAAAATTATCGCTAAAGCTAAAGAA
GTCGGTGAATACCCAACATTAGGTTCTAACTGGACTGCAGAAATTTCTTCGTCTTTCTCT
GGTTTAGCCGCAGTAATAACTCTTGGAAAAATTATTACTGATTCTGGTATTTTATTAGCT
GAAATTGAAAATGCTGAAGCTGCTATGACAGCGGTTGACTTTCAAGCAAATCTCGAAAAA
TACGGAATTCCAGGAGTAGTAGCTCTTTATCCAGGCGAATTAGGCGATAAAATTGAAATT
GAAATCGTATCTAAAGCTGACTATGCAAAAGGAGCTTCTGCATTACTCCCAATTTATCCA
GGTGGTGGTACTCGTGCATCTACTGCTAAAGCAGTGTTTGGATATGGACCACAAACTGAT
TCACAGTACGCTATTATAGTTCGTCGTAATGATGCTATTGTTCAAAGTGTTGTTCTTTCA
ACTAAGCGCGGTGAAAAAGATATTTACGACAGTAACATCTATATCGATGACTTTTTCGCA
AAAGGTGGCTCAGAATATATTTTTGCAACTGCACAAAACTGGCCAGAAGGATTCTCTGGA
ATTTTAACTCTGTCTGGTGGATTATCATCAAATGCTGAAGTAACAGCAGGAGATTTGATG
GAAGCTTGGGACTTCTTTGCTGACCGTGAATCTGTTGACGTTCAACTGTTTATTGCGGGT
TCTTGTGCCGGTGAATCTTTAGAAACAGCATCTACTGTCCAAAAACACGTCGTTTCAATT
GGGGATGCTCGTCAAGATTGCTTAGTATTGTGCTCACCACCACGTGAAACTGTAGTTGGA
ATTCCTGTAACTCGTGCAGTAGATAATTTAGTTAACTGGAGAACTGCGGCAGGTTCATAC
ACTGATAATAACTTTAATATCAGTTCAACCTATGCAGCAATTGATGGTAACTACAAATAT
CAGTATGACAAATATAATGATGTGAATCGTTGGGTTCCATTAGCAGCTGATATTGCTGGT
TTATGCGCAAGAACTGATAACGTATCTCAGACTTGGATGTCTCCAGCTGGTTATAATCGT
GGCCAGATTCTTAACGTTATTAAACTTGCTATTGAAACTCGCCAGGCTCAGCGCGACCGT
TTATACCAAGAAGCTATCAACCCGGTAACTGGTACAGGTGGTGATGGTTACGTATTGTAT
GGTGATAAAACAGCTACTTCTGTTCCTTCTCCGTTTGATCGTATTAACGTTCGTCGTCTG
TTTAATATGTTGAAAACGAATATCGGACGTAGTTCAAAATATCGTTTGTTCGAATTAAAC
AACGCGTTTACTCGTTCATCATTCCGCACGGAAACTTCCCAGTACTTGCAGGGAATTAAA
GCTCTCGGTGGAATTTATGAATATCGTGTAGTTTGCGATACAACAAATAACACTCCGTCA
GTAATTGATAGAAATGAGTTTGTTGCAACATTCTACATCCAACCGGCGCGCAGTATAAAT
TATATTACTTTGAATTTCGTCGCAACTGCTACTGGTGCAGACTTCGATGAGTTAACTGGT
CTTGCAGGTTAATTGATTATGGGGCTTCGGCCCCTATTCTCGAGGAAACAAGTGATTATT
TCTAAGTTTGTTAAAGTGAAAATTGTACCATCAAATTTTAAACATTATCAATCAAAGGGT
TATAAATTAACACCTCTTGGAGCGGTTAAACATGGATTTAGGTATTATGAAATTGATGTT
AAAGTTGAAGACATTAAAGGTAGTACTGCGTATGTAAAATGCCGTTGCGATAAATGTGGA
ATTGAATATAACCAACGCATTGGAAGAAACACTGATATTTGTCGTGATTGCCGTCATAAA
GATAAAATGCGCAATAATTCATACGGTTCCGCGAATAAAGGAAAAACAGTAGAATGTATG
CATGGCGTAAATCATCCAAGATGGAACCCAAATAAATCTGAAATGCGTGAATATATTTCA
TTGGTATATAAAGAAACTCGTAAGCATAAGAAAATTTGGTCAAAATGGCCAAATGCTGAT
AAAATAGGAGTGTGTGGTACAAAAGGTGCTTACCAACTTGATCATAAGGTATCTATCAAA
TACGGTTTTGATAATTTAATTCCTGCTAAAATAATTGGCGGTATTAATAATTTAGAAATA
ATCACTTGGGAATCTAACCGAGAAAAATCAAAAAGAAATAGTGTAGATCTTTGGGATTTA
CTAGAATGATTCTAAAGGCCTGTTTCGGCAGGCCATATAAATACACTATATCCTTAATTC
TTTAATTCTATATGCCCTAGGTTAAACATAGGGATATAAATACTACAGAGGCTAATATGT
TTGTAGATGATGTAACACGAGCGTTTGAATCTGGTGATTTTGCTCGACCTAACTTATTCC
AAGTAGAAATTTCTTATCTTGGACAAAATTTTACGTTCCAATGTAAAGCTACTGCTCTAC
CAGCTGGTATTGTAGAAAAAATTCCAGTCGGATTTATGAACCGTAAAATTAACGTAGCAG
GTGATCGTACATTCGATGACTGGACTGTTACAGTAATGAACGATGAAGCTCATGATGCTC
GCCAGAAGTTTGTTGATTGGCAAAGCATTGCTGCCGGTCAAGGAAACGAAATTACTGGTG
GAAAACCCGCAGAGTATAAAAAGAGCGCTATCGTTCGTCAATATGCTCGTGACGCTAAAA
CAGTAACAAAAGAAATTGAAATTAAAGGTCTGTGGCCTACTAACGTGGGTGAACTTCAAT
TAGATTGGGATTCAAACAATGAAATCCAAACCTTTGAAGTAACTCTTGCTCTCGATTATT
GGGAATAAAATGAATGGGGAGAAATCCCCATCCTGCTTAAAGCAGAGAAGTCCATTATAA
ATATAACTATAATTCCCATTTGGAGAATACAATGAAATTTAATGTATTAAGTTTGTTTGC
TCCATGGGCTAAAATGGACGAACGAAATTTTAAAGACCAAGAAAAAGAAGATCTTGTTTC
CATTACAGCCCCAAAGCTTGATGATGGAGCGAGAGAATTTGAAGTAAGCTCGAATGAAGC
TGCTTCTCCTTATAATGCTGCATTCCAAACAATTTTTGGTTCATATGAACCAGGAATGAA
AACTACTCGTGAGCTTATTGATACATATCGTAATCTCATGAATAACTATGAAGTCGATAA
TGCAGTTTCAGAAATCGTTTCAGATGCTATCGTCTACGAAGATGATACTGAAGTCGTAGC
GTTAAATTTGGATAAATCTAAATTTAGTCCAAAAATTAAAAATATGATGTTAGATGAATT
TAGTGATGTATTAAATCATCTATCGTTTCAACGAAAAGGTTCTGATCATTTTAGACGTTG
GTATGTTGATTCAAGAATTTTCTTTCATAAAATCATTGATCCAAAACGTCCAAAAGAAGG
CATAAAAGAATTACGTAGATTAGACCCTCGCCAAGTTCAGTATGTTCGTGAAATTATAAC
AGAAACTGAAGCTGGCACAAAAATAGTTAAAGGTTACAAAGAATATTTTATATATGATAC
TGCCCATGAGTCATATGCATGTGATGGTAGAATGTATGAAGCTGGCACAAAAATAAAAAT
TCCTAAAGCTGCCGTCGTTTATGCCCATTCTGGATTAGTCGATTGTTGCGGTAAAAATAT
CATCGGGTATTTGCATCGTGCTGTTAAACCTGCTAACCAATTAAAATTACTAGAAGATGC
TGTAGTCATTTATCGTATTACTCGTGCTCCTGATCGTCGTGTTTGGTATGTAGACACAGG
TAATATGCCTGCTCGTAAAGCTGCCGAGCACATGCAACATGTCATGAACACGATGAAAAA
CCGTGTAGTATATGATGCATCAACAGGTAAAATAAAAAATCAACAGCATAATATGTCTAT
GACCGAAGACTATTGGTTGCAGCGCCGCGATGGTAAAGCTGTGACAGAAGTTGATACTCT
TCCTGGTGCTGATAATACCGGTAATATGGAAGATGTTCGTTGGTTTAGACAAGCTCTTTA
CATGGCATTACGTGTTCCTCTTTCACGCATTCCGCAAGACCAACAAGGCGGTGTGATGTT
TGATTCTGGAACTAGCATTACACGTGATGAATTAACGTTTGCTAAATTTATTCGTGAGTT
GCAGCACAAGTTTGAAGAAGTTTTCCTAGATCCGCTTAAAACAAATCTTTTGCTTAAAGG
TATAATTACAGAAGATGAGTGGAATGATGAAATAAATAATATTAAGATAGAATTTCATCG
GGATAGCTACTTTACTGAACTCAAAGAAGCAGAAATTTTGGAACGAAGAATTAATATGCT
AACCATGGCAGAACCATTTATTGGTAAATATATTTCTCACAGAACTGCTATGAAAGATAT
TTTGCAGATGACTGATGAAGAAATAGAACAAGAAGCCAAGCAAATTGAAGAAGAGTCTAA
AGAGGCTCGTTTCCAAGACCCCGACCAAGAACAAGAGGATTTTTAATGGAAGGTTTAATT
GAAGCTATTAAATCAAACGACCTCGTAGCCGCTCGTAAATTATTTGCTGAAGCCATGGCT
GCAAGAACGATTGATTTAATTAAAGAAGAAAAAATCGCTATCGCTCGTAATTTCTTAATC
GAAGGTGAAGAACCTGATGACGAGGATGATGACGAAGACGAAGATAGTGATGATAAAGAC
GACAAAAAAGACAAAGACTCTGATGAAGATGACGAAGACGAGGATGATGAATAATGCTTC
TGATCCCTGAAACTCATGAATTAGTTCTCGAGAATGTCGAAGCACTTATTCCTGAAGCAC
AGGGTCGCTTTGACGAATTGTCTTCTGCTTTAAATAAAGACGATATAAATACAATTGTCG
AGAATATGCTTGATGATGAAACTGATTTAGCGGTTGCATTAGCTTCTATTAATGAAAATA
TGCCGTTAAATGAATTTATCGTTAAACATGTTTCTGCCCGTGGTGAAATTACTCGCACTA
AAGATCGCAAAACCCGCGAACGTAATGCATTTCAAACTACCGGGTTGTCTAAAGCAAAAC
GTAGACAAATTGCTCGTAAAGCTACCAAAACGAAGATTGCCAATCCAGCAGGTCAATCTC
GTGCTCAGCGTAAGCGTAAAAAAGCTCTTAAACGCCGTAAAGCATTAGGATTAAGCTAAT
GAATGAACCCCAATTACTAATTGAAACTTGGGGTCAACCTGGCGAAATTATTGATGGCGT
ACCAATGCTTGAATCTCATGATGGAAAAGACTTAGGTTTAAAACCTGGTTTATACATCGA
AGGAATATTCATGCAAGCGGAAGTCGTCAATAGAAATAAACGTCTTTATCCAAAACGTAT
ATTAGAAAAAGCGGTAAAAGACTATATTAATGAGCAAGTTTTAACTAAACAAGCTCTCGG
AGAATTAAATCATCCTCCACGCGCTAATGTTGACCCGATGCAAGCCGCTATCATTATAGA
AGATATGTGGTGGAAAGGAAATGACGTATACGGACGAGCTCGTGTTATTGAAGGTGACCA
TGGTCCTGGAGATAAATTAGCAGCTAATATTCGTGCCGGATGGATTCCAGGAGTTTCTTC
TCGTGGATTAGGTTCATTGACTGACACAAATAAAGGTTATCGTATCGTAAACGAAGGATT
CAAATTAACTGTAGGTGTTGATGCAGTATGGGGTCCAAGTGCTCCAGATGCATGGGTAAC
TCCTAAGGAAATTACCGAATCACAGACGGCGGAAGCCGATACAAGTGCCGATGACGCCTA
TATGGCTCTCGCAGAGGCCATGAAAAAAGCGTTATAAATATTATTATCTAAACAACAGGA
CTACAAAATGCTTAAAGAACAACTGATTGCCGAAGCGCAGAAAATTGATGCTTCCGTTGC
TCTTGATAGTATTTTCGAATCAGTTAATATTTCTCCGGAAGCAAAAGAAACTTTCGGCAC
TGTATTCGAAGCTACCGTCAAGCAGCACGCCGTTAAATTAGCTGAATCTCATATCGCTAA
AATTGCTGAAAAAGCAGAAGAAGAAGTAGAAAAAAATAAAGAAGAAGCCGAAGAAAAAGC
TGAGAAGAAAATCGCTGAGCAAGCTTCTAAATTCATTGACCATCTTGCGAAAGAATGGCT
CGCTGAAAATAAATTAGCAGTTGATAAAGGCATCAAAGCCGAACTGTTTGAATCCATGCT
TGGTGGATTAAAAGAGCTCTTTGTTGAACACAACGTTGTTGTTCCAGAAGAATCAGTTGA
TGTTGTAGCTGAAATGGAAGAAGAGCTGCAAGAACATAAAGAAGAATCAGCTCGTCTGTT
CGAAGAACTCAATAAGCGTGACGCATATATCAATTATGTGCAGCGTGAAGTGGCATTGAG
CGAAAGTACTAAAGATCTGACTGAGTCTCAAAAAGAAAAAGTCTCTGCTCTGGTCGAAGG
TATGGATTATTCAGATGCATTCTCAAGTAAATTGAGTGCAATCGTAGAAATGGTGAAGAA
ATCTAATAAAGATGAAAGCACTATTACTGAGAGTATAAATACTCCTGATACTGAAGCAGC
CGGACTGAATTTCGTCACTGAAGCTGTAGAAGATAAAGCTGCACAGGGTGCAGAAGATAT
TGTAAGTGTATATGCGAAAGTCGCATCTCGTTTCTAATTTTAAAGGTTAACACAAATGAC
TATCAAAACTAAAGCTGAACTTTTGAACAAATGGAAGCCATTACTGGAAGGTGAAGGTTT
ACCGGAAATTGCTAATAGCAAACAAGCGATTATCGCTAAAATCTTTGAAAACCAGGAAAA
AGATTTCCAGACAGCTCCGGAATATAAAGACGAAAAAATTGCTCAGGCATTTGGTTCTTT
CTTAACAGAAGCTGAAATCGGCGGTGACCATGGTTACAATGCTACTAACATCGCTGCAGG
TCAGACTTCTGGCGCAGTAACCCAGATTGGCCCAGCTGTTATGGGTATGGTACGTCGTGC
TATTCCTAACCTGATTGCTTTCGATATTTGTGGTGTTCAGCCGATGAACAGCCCGACTGG
CCAGGTATTCGCACTGCGCGCAGTATATGGTAAAGACCCAGTGGCTGCCGGTGCTAAAGA
AGCATTCCACCCAATGTATGGTCCAGATGCAATGTTCTCTGGTCAGGGTGCTGCTAAGAA
ATTCCCAGCTCTGGCTGCTAGCACACAAACCGTTGTAGGTGACATCTATTCTCACTTCTT
CCAGGAAACTGGCACTGTATATCTGCAAGCTTCCGCTGTAGTAACAATTGATGCTGGTGC
GACTGATGCTGCTAAATTAGATGCCGAAGTTAAGAAACAAATGGAAGCTGGTGTACTGGT
AGAAATCGCTGAAGGCATGGCTACTTCTATCGCTGAACTTCAGGAAGGTTTCAATGGTTC
TACCGATAACCCATGGAATGAAATGGGCTTCCGTATCGATAAACAAGTTATCGAAGCTAA
ATCTCGCCAGCTGAAAGCTGCTTACTCTATCGAATTAGCACAAGACCTCCGCGCTGTTCA
CGGTATGGATGCTGATGCTGAACTGTCTGGTATTCTGGCTACCGAAATCATGCTAGAAAT
CAACCGTGAAGTTGTTGATTGGATTAACTACTCCGCACAGGTTGGTAAATCTGGTATGAC
CCTGACTCCGGGTTCTAAAGCTGGTGTATTTGACTTCCAGGATCCGATTGATATTCGTGG
TGCTCGCTGGGCGGGTGAATCCTTTAAAGCTCTGTTGTTCCAGATTGACAAAGAAGCAGT
TGAAATTGCTCGTCAGACCGGTCGTGGTGAAGGTAACTTCATTATCGCTTCCCGTAACGT
AGTTAACGTTCTGGCTTCAGTTGATACCGGCATTTCTTATGCTGCACAGGGTCTGGCTAC
CGGCTTTAACACTGATACTACCAAGTCAGTATTTGCTGGTGTTCTGGGTGGTAAATACCG
CGTATATATCGACCAGTATGCTAAACAGGATTATTTCACTGTAGGTTATAAAGGTCCGAA
CGAAATGGATGCTGGTATTTACTACGCTCCATATGTAGCGCTGACTCCGCTGCGTGGTTC
CGATCCGAAGAACTTCCAACCAGTAATGGGATTCAAAACTCGTTACGGTATCGGTATCAA
CCCATTTGCAGAATCCGCTGCTCAGGCTCCGGCTTCTCGCATCCAAAGCGGTATGCCTTC
TATTCTGAATAGCCTTGGTAAAAACGCTTACTTCCGCCGTGTATATGTTAAAGGTATCTA
ATCTTTGACGATTTAAGGGACCTTCGGGTCCCTTTAGTTGTTTTTATGGTATAAATAAAT
CATATAAACCAAAAGGAAAGCGCAATGGCTAAAATCAACGAACTTCTGCGCGAATCAACC
ACAACGAATAGCAACTCAATCGGTCGCCCAAATCTCGTTGCTTTGACTCGCGCTACCACT
AAATTAATATATTCTGACATTGTAGCAACACAAAGAACTAATCAACCTGTTGCTGCTTTT
TATGGTATCAAATACCTTAACCCAGACAATGAATTTACATTTAAAACTGGTGCTACTTAC
GCTGGCGAAGCTGGATACGTAGACCGAGAACAAATCACAGAATTAACAGAAGAGTCTAAA
TTAACTCTCAATAAAGGCGATTTATTCAAATATAATAATATCGTTTATAAAGTATTAGAA
GATACACCATTTGCTGATATTGAAGAAAGCGATTTAGAATTAGCTCTTCAGATTGCAATC
GTTCTTTTAAAGGTTCGTCTATTTTCTGATGCTGCAGTAACAAGCAAATTTGAAAGCTCT
GATAGTGAAATTGCGGATGCTAGATTCCAGATTAATAAATGGCAAACCGCGGTTAAATCT
CGTAAACTTAAAACTGGCATCACAGTTGAATTAGCGCAAGATTTAGAAGCAAATGGATTC
GATGCTCCTAATTTCTTAGAAGATTTGCTTGCAACTGAAATGGCAGATGAAATCAATAAA
GATATTCTGCAGTCTTTGATTACAGTGTCAAAACGCTATAAAGTTACAGGAATTACTGAT
ACCGGATTCATTGATTTGAGTTATGCATCTGCTCCTGAAGCTGGTCGTTCATTATACCGA
ATGGTATGTGAAATGGTTTCGCATATCCAAAAAGAATCAACTTATACAGCAACGTTCTGT
GTAGCTTCTGCTCGTGCCGCCGCGATTCTTGCTGCATCAGGTTGGTTAAAACATAAACCA
GAAGATGATAAATATCTTTCACAAAATGCCTATGGGTTTTTAGCTAATGGTTTACCACTT
TATTGCGATACTAACAGCCCATTAGATTATGTAATCGTTGGCGTAGTAGAAAATATCGGT
GAAAAAGAAATTGTTGGATCAATTTTCTATGCTCCGTATACAGAAGGTCTCGACTTAGAT
GACCCTGAACATGTAGGTGCATTTAAAGTTGTTGTTGATCCAGAAAGCTTGCAACCGTCT
ATCGGTTTATTAGTTAGATATGCTTTATCTGCAAATCCTTACACCGTAGCAAAAGATGAA
AAAGAAGCAAGAGTAATTGATGGTGGAGACATGGATAAAATGGCGGGTCGTTCAGATTTG
TCTGTTTTATTAGGTGTTAAGCTACCAAAAATTATCATTGATGAATAAAACAAAGGGACC
TTTCGGTCCCTTTTTATTTAACTTACCAATTCAATTCAATCCAAGCCGGACGAAGTACAT
CTTGTACCATTTTAACTAATTCCTTTTTAATCAAAGAAGGATTATCCGCTTGAGTTAGAG
TAATACCTTCACGAGAAGTTTCTTCCAAAATATCTTGAACAGTTAGTCCCATCACCTTTC
CAAAATCTTTTGGACCAATTTCGCCAATTTTAGAAATAACGTTATTTACTCGGTTTAGTG
TAACGTAACAAGCTAAAATCCCCACCAATTTGTTATCAGCTTCTGACAGTTCAACTTTAG
CTTTAATAGGCTTATCAGACTTTTTCTTTTCACTAAATTTAGAATTCTTGCATTTGATGG
CTACACGATTGCGATTAGGCAGCCAAGAAGGATAGCAAGGTTTCAATACATATCCTTCAG
CGGTAAATACTTCGCCTTTGGCTTCAGCTTTCCAAACACATTTGTTAGCGTCAATTAAAC
CTGCATTATCTACGGTAAAATTATAAGACGGAACAACAGAATCCAAATCATTTGGAAGTT
TAATAAGATCTTCAAATTTACCACGACCCAAAAGTGGAGCGATTTTAAACCCAAATGAAT
TACAAAACGATTCCATCATGTAATCATCAACATAAGTTACATCACCACTTTCGGTAGTGA
CAATAATGTCAAATACATAAAAATCTTTATCGCCATAATCAACATTCTTCTGAATGCCAG
GTCCAGCGAATTCGCCAAAAACTTGATAAGTCACAACAGCTGAAGTTTCCATAATTTCTT
GGACAGCTTTAATAGAATCTTCGTAATTCTTTAAAATAATTTCATAACCGAAGAAATCTT
CAGCAGGAAGAATCGGTCCAGTACGTTTAGCGCAAGTCACTTTATCACGCTCAATAATCA
ACGAGAAATTTGTGCCGTGAATCTTTTCACGAGCTACCCATTCGCCGCCAGTCAATCCCA
AGCTGTAAAGTTTTTCAATAAATTTAGAGTTGTAATGATTTTCAAGACTGCTATACTTTT
TAAACATAATTAATCCTCAAAATGTAATTTCTAACCAATCACCATCACGCTGATCACTAT
TGACTTTAAAGCTGAATCCTTCTTTTCTCAGCCAATCACCAATTTCTTCAGTAATCAATT
TATCGCGAGCAGTACAATAATAACTAAAATATGTTTTACCTTGTTCAGCTGCTTTATTAG
CAAGTTCTGAAAAATCTTTAATAAAACACTCTAGCTTAAACTGTTTACTTTTTAATGCTT
TTTCACGTAATTGATTAGCAAAAGATTCATTTTCATAAAGATCATACTGTTCCATTTTTC
ACCTTTTTATTGATATGTCTTTTTCTATAGACAACTTTTTCTCGAGTTTCAAATTTATTC
TTAGCAGTCATAGCTCGAGCCCATAATACAGCCACTTCTTTCGCCTGTAAGTTTAATTCA
CGAGCAATTTCAATGAATGACTTTCCAGACTCATGAAGAGTAAACACCACAACCTCAGTT
CTCATAATCAATCTCATGTTATCGAGTTGGTGCCATTATATACACCATTTTCTGATTGTG
TTTTGTGTGCTTTCAAAATGAAAAAAGGGCCGAAGCCCCTTATGATTATGGATAGGTATA
GATGATACCAGTTTCTAAAGCAGTTTTGTGAATGATGTATCCATTACGTGATTCTTGAAC
ATCAACTTCTGGATAGTCTTTCATCATTTTCTGAAGAGTGTAACGATGCAAGTAGTACTT
ACTATCTGGATCATCAGTTTTCCAATCTTTACCTTCTTCGGTCATTTTTTGGATTTCATC
CATAACCCACCAACCGCACCAGATGTAAGCTGAGCTACGGTGTGGAAGAGGATGAACATA
AGGTAATTCACCTTCTGGTTCTGGAGCAACTTTATCAGTCACTTGAACTTGACCTTCTGC
TGTAATAGTTTTACTATCATAATCAGTTGCCGTAACGATAGCAGTAACTTCAATAGTTTG
ACTTCCAACAGATGACGTATCAACGGTATACACATTAGTTGATCCTTCTACGGGAGAAGA
ATCTTTCTTCCATGAATAAGCAATTTGTGCTTCTTCTGGTGCATCAGTGACATTAGCAGT
AAATGTAGCCGAAGCATCTTGTTGAACGTTAATAGAAGGAGGAGTCAATGTAACCTGTGG
ATTCATTGTCTTCTTATTAACTGTTAATGATACTTCATTCGAAGTAACTTCCTTTTCATT
GTAATTTTCAGCAGTTACTTGGGCTACGCATTTAATTCTTTTTACTCCACTTGTGGTAGG
AGTGTAATTGAATGTAGCAGAAGTTGCTTCGCCTACAGGAGAACCGTCAACGTGCCATTG
ATACGTAGCAGACGCTCCAACTGGTTGAGAAGATAAGGCAGCAGTAAATTGAACTGGGGT
TCCAATTACTCCAACTGAAGGACTATCAGGAGTTACAGCTAAAGTAGTTGTCTGTGTCTT
ATTTTGAACTGTGATAGTTGTAGTAGCTTCTGCTGTTTCAGCATCAGTATCTGGAACTGG
GTTTGTTGCAACTACTTTAATAGTTTTTTGACCGGCAGGTCCTTTTAGTACATAACTAAA
AGTTGCTGACGTTTCTTCCTGCGGAGCGTCGTCTACAGTCCAAGCATATGTAATAGGTCC
ATCTCCAGTTTGACCACTGGGTGTAGCAGTAAACTGCTGAGTCTGATCTATAACTCCAGT
AGGCGTTTTAGGAGTTATATCAACTGTAAAAGTCATAAATTATCCTTATTTTAATGTTAC
GAAAGAAGAATTACGTGTTTCACGAATTAGAACTGATCCGTCACGATTAATGTAGTAAAT
TAAGCTAAATAAAGTTTGGTGTGCTGACGCATGCTCAAAACTTGTTGGGTGAGATTTCCA
ATCCGGAGTTTCAGCAATCCATTGATAAATCCACCAAGGAACAGTACAGAATCCTGGATT
TTTTCCAATCAACTGAAGATTCGGGCTAAAGTTTTCCGGAAGAGTAAATACAGACGGATT
TTCAGATTCAATAATTTCAGCTACAGCCTGTTCAAATTTTTCTTCAACAAAAGGAGTATC
TTCAATCAAAACATCGGTATTTTCAGGAATTTTATCCGTTTCTACTACTTCAATTTTAAT
GTCAGATTTAACCGGAGAATCAATCAGAAGTGCTGCTTCTGGATTGACTTCTTCATCGTC
ATATTTTAATCCCTCTGCAGCATCAGCAGCATCAATTAAGTCTTTAATAGATAACCCATC
AGTCTCTGGCATAGGTTCACTAGCGAGCTTCTGGAGGGCTTCTTCAATATCATTAACGAT
ATTATCAAAAGATTTATTCTTTTTGACCTTTATACCAAACTGTTCAGCATATTCAGCTAA
TTTAGCTTTAGCTTCTTTGTTATCATCAAGAGCCTTCAGCTCTGCAATATAATCTTTATC
TATCATAATATTTCCTCAGTATAAATATAGATATATTTATTACTCGGAAAATAGTATGTA
CCACTTTGTATATGAAACAACAAATCTAATAAATGGTAAAAAGTATATAGGAAAACACTC
TACTGATGACTTGAATGATGGTTACCTTGGTTCCGGTAAGGCAATTCAGCAGGCTATAAA
GAAATATGGTGAAAACAATTTCTCTAGAACAATACTAAAAGAGTTTAAAACTTCCGAAGA
AGCGTACATGTATGAAGAAGAAATTATAACTCCTGAAATAATAAAAAGCAAAAATTATTA
TAATATGAAACCTGGCGGAATTGGTGGAATTGTTATGACTACAGAGGTTATAGCAAAGAT
GAAAGAATCTTCCGCTAAGAGATTTGAAAACTCACCGGGCACGGTATTAGGTACAACTTG
TTACACTAATGGAACTAAAAATATTTTTATTAAGCCTGGAGAACTTGTTCCAGAAGGATT
TGTAAAAGGGATGGTTCATCCTAATAGAAAGTCCAGAAAAGGATGTAAAGTCAAACCGAC
TACCACAGGAACTTTTTGGGTCAATAATGGTGCAATAAATAAATTAATACAACCAGACGG
TATTATTCCCGACGGATTTATTAAAGGTCGTCTCATGAAAAGAGATTCTAAAGGCAAATT
TAGTAAGGCATAATTATGGATATTAAAGTACATTTTCACGACTTCAGTCATGTACGCATC
GATTGTGAAGAGAGCACGTTCCACGAATTAAGAGATTTCTTTTCGTTTGAGGCCGATGGA
TATAGATTTAACCCTCGCTTCAGATATGGCAACTGGGATGGACGAATCCGTCTTTTAGAT
TATAATCGTCTTCTTCCATTCGGCTTAGTCGGGCAAATTAAAAAGTTCTGTGATAACTTT
GGCTATAAAGCCTGGATTGACCCACAAATTAACGAAAAAGAAGAATTATCAAGAAAAGAT
TTTGATGAATGGCTTTCTAAATTAGAAATCTATTCAGGAAATAAAAGAATTGAACCACAC
TGGTATCAAAAAGATGCAGTGTTCGAAGGATTAGTTAATCGTCGTAGAATTCTTAATCTT
CCAACATCTGCAGGTAAATCTTTAATTCAAGCTTTGCTTGCGCGATATTATTTGGAAAAT
TATGAAGGTAAAATTCTTATCATTGTTCCAACAACCGCTTTAACAACTCAGATGGCTGAT
GACTTCGTTGACTATCGTTTATTCAGTCATGCAATGATAAAGAAAATTGGTGGCGGAGCA
TCAAAAGATGATAAATATAAAAATGATGCACCAGTCGTTGTTGGTACATGGCAAACTGTA
GTAAAACAACCGAAAGAATGGTTCTCACAGTTTGGAATGATGATGAATGATGAATGCCAT
CTTGCTACAGGAAAAAGTATTTCATCTATCATATCAGGTTTAAATAACTGCATGTTCAAA
TTTGGTCTATCTGGTTCATTACGTGATGGCAAAGCCAATATCATGCAGTATGTTGGAATG
TTTGGTGAAATATTTAAGCCAGTAACGACTTCTAAGTTAATGGAAGATGGACAAGTAACT
GAGCTAAAAATTAATAGTATTTTTCTTCGCTATCCCGATGAGTTCACTACTAAATTAAAG
GGAAAAACTTATCAAGAAGAAATAAAAATTATTACGGGGCTTAGTAAAAGAAATAAATGG
ATCGCTAAATTAGCTATTAAGCTTGCGCAAAAAGATGAAAACGCTTTTGTCATGTTTAAA
CATGTATCACATGGTAAAGCTATTTTCGATTTAATTAAAAATGAGTACGATAAAGTTTAT
TACGTATCAGGGGAAGTTGATACCGAAACCCGCAATATAATGAAAACCTTAGCTGAAAAT
GGTAAAGGAATAATTATAGTAGCTAGTTATGGTGTATTTTCTACTGGTATTTCAGTTAAA
AATCTGCATCACGTTGTTTTAGCGCATGGTGTTAAATCAAAAATCATTGTATTACAAACA
ATCGGTCGTGTATTACGTAAGCATGGTTCTAAGACAATAGCAACAGTCTGGGACCTCATA
GATGATTGTGGTGTTAAGCCAAAATCTGCTAATACGAAAAAGAAATATGTTCATTTAAAC
TATCTTTTAAAACACGGCATTGATCGTATTCAGCGCTACGCAGATGAAAAATTTAATTAC
GTAATGAAAACAGTTAATTTATAAGGGCTTCGGCCCTTTGGAGAAAAAGATGTTACTAGA
ATTTAAACAATTTCTTTATGAAGCTTCTATTGATGAATTTATGGGTAAAATTGCCTCTTG
TCAAACATTAGAAGGTTTAGAAGAACTTGAAGCTTATTATAAGAAAAGAGTTAAAGAAAC
TGAATTAAAAGATACTGATGACATCTCAGTCAGAGATGCTTTGGCAGGAAAAAGAGCTGA
ATTAGAAGATTCAGACGATGAAGTAGAAGAAAGCTTTTAAATTAAAAAAGGCCCAACCAA
AAAGGAAGGGCCAAAACTATAGACTAAAGGTCACACTATAGCAAAAGTTGTGTTTCATTT
AATTGTTCTTCCGAACTTTCTGAAACTGGTAGTTCTTTAATGTAATTATAGCAAGGCCCA
GGATGTACAGGACCTTTGTCTGTTTCAACAACCAACGCAGAATCGATTGGAGTTTTACAG
ACAACACAAATCTTATCTGACATGATTGTCTCCTCTGAATTATATATCTATTTATACAAC
TCTCATATGCATATCAATGCCCATATCTTTAGAATAAAAATATTCATCAAGATATCCGGC
AAATTTTCCTTTAACATAAAGAACATCTTCACCACACGGGTGGTCGGCTAGGATACGAAT
ATCCTGACGCTTAAGATTATGCTTTTTCATTAAGAATTGAATTTCCGTTTCAAATTCTTC
TTCATAATTAAAAGCATCATCAATGCTATATCTCATTATTTTCCAGCCTCAAATGCCCGC
ATGTCTTGAATATGCTTAATAGCAAATCCACGTGATTTAATAGCATCAAGAGCTCCGCTA
CAGAAATCTAATAAAATTCCCCAATATTGTAATGAAGTATCAACCTTTAAAACATCCTTA
TCAGCTGATAGAACTGTCTTCATTTCTGATTTCTCGTAACGATCCATACTAAATTCATCA
CCATCTCCTCGTCCCGAGTAGTAGTCTAATCTAGCTTTAAGAGCAACTTTTTTCTGTGCG
TCAATTCTAAGCATTTCCTTTTTAATACTTGAATGCTTATTAAGCCATTTACTATATAAC
ATCACATTATTAGCTGCTTCATACTGTAATTTAGTCGAATCTATAAACACATCTTTCTTC
AATTCTTCTTGAAGATCTTCTAATCTCATATTGTTCTCTATTCAATTGTTATTGGATGGA
CTTAGATTCATTATACCACGTTTATACGTGAAGCATTATACTCTATTACTGGAAGCCAGT
TGTAGTTTTATCTGCTCAATATCATCAGGGTTATCGATGACTGAAAAGCGTATTTCTACT
ATCAGAGTATAATCATCATAAACCGGTATTACATTAACTGCTAATTTATCAATACGTGGC
TCATAGTTTCTTACTGCGCTTTCGATATTACGCTCAACCGTGTCAGCAGTAAGAGGAGTC
ATATTCTCAAAAAGTTGGTCAGATAAATCGCATCCAAATTCAGGGTCAAACGGTCTTGAA
CCTTTTCTTGTTGTAATAATTCCTAAAAGACTATTTTTAATTGACCTTAATCCAAGTGAT
CTTGAAACGTCTTTGTTCCAATCCATTTTCATCTCTGGGTCAATATCAGAATAAAGCTTA
TTAATATTTGCCATTACAATAGCTCAAAGAATTCTTTGAGTCCTCTTATTACATGAGCGT
GAGTTTTTCCACACTCTGGACACTTAATTGGAACAGCCAAATAAACAGTAGGCTTTAAAA
GCATATCTTTTATAGCTACAATATCTGACTCTGTGATGATAGAATATAAATCTTCCAATT
CTTTTTCATTTAAGTCTTCAACTGGAATGCTTTCACCGTTAGCATGAATCGTTTCTATAC
ATGATACTATCATGTGGGCTATATTTTTATCATCAAAAATTTTAGGGTAATGGAATTTAA
TTTTAATGTCACCCAGTGTATACCAGAGGTCTTCTGATGCATCTATTTGTGTATGTAATA
GATTTATATGGGTTGGTATTTCAGTTCCACAGGTGCACTTCCAAGAGTTTTCGTGATTAA
CTTCACCAAGAGAATGCGCCCATAAATGAATCAGTAATAGTTCTGATTCTTGGCGGTTTA
AATCTTTTGCATTTGTACAGTCTTTGATTAGCTTTTTAACAATCTCTTCTACTGAACCAT
TCTTTTTAGCAGTAATAAGTTCTAGATATTCTTTAAGAGTAAATGCGCGACAGTTGATTA
TTTTAGAACCAACTCTCACATCAAATTTGTATTCATACATATTTAGCTCCTTTATTTATC
ATATTTATAAATAGAATAAAAGGAGCATCTATGGCAAACATTATTCGTTGTAAATTACCA
GATGGTGTTCATCGTTTTAAACCATTTACGGTAGAAGATTATCGAGATTTTTTGTTAGTT
CGAAACGATATAGAACATCGGTCACCACAAGAACAAAAAGAAGTAATTGCTGATTTAATT
GCTGATTATTTTGGAGACTATCCAAAGACTTGGCAACCATTTATATTTTTGCAGGTATTT
GTAGGTTCAATAGGTAAAACTAAAGTACCAGTCACATTTGTATGTCCAAAATGTAAAAAA
GAAAAGACAGTTCCATTTGAAATATATCAAAAAGAATTAAAGGAACCTATTTTTGATGTA
GCTAATGTTAAAATTAAATTAAATTTTCCTTCTGAGTTTTATGAAAATAAAGCAAAGATG
ATTACTGAAAATATTCATTCTGTTCAAGTCGATGAAATGTGGTATGATTGGAAGGAAATT
AGTGAATCAAGTCAAATAGAACTTGTTGACGCCATCGAGATAGAAACATTAGAAAAAATT
CTCGATGCAATGAATCCTATTAATTTAACATTGCACATGTCATGTTGTAATAAGTATGTA
AAAAAATACACTGATATAGTAGATGTGTTTAAGCTATTAGTTAACCCAGATGAGATATTT
ACTTTTTATCAAATTAATCACACACTCGTAAAAAGTAATTATAGCTTAAATTCAATAATG
AAAATGATTCCTGCTGAGCGCGGATTCGTATTAAAACTGATTGAGAAGGATAAACATCAA
TGAGTATGTTACAACGCCCTGGATATCCAAATCTCAGCGTTAAATTATTTGAGAGTTATG
ACGCTTGGAGTAATAATAGATTTGTTGAATTAGCTGCTACTATTACCACATTAACTATGC
GTGACTCTCTTTATGGTCGAAATGAAGGAATGCTACAATTTTATGATTCTAAAAACATTC
ATACAAAAATGGATGGAAATGAAATAATTCAGATTTCTGTAGCTAATGCAAACGATATTA
ATAATGTTAAAACACGAATTTATGGATGCAAGCATTTTTCCGTGTCAGTAGATTCAAAAG
GCGATAACATCATTGCTATTGAATTAGGAACTATTCATTCTATAGAAAATCTTAAATTTG
GTAGACCATTTTTCCCTGATGCCGGTGAATCTATAAAGGAAATGCTTGGCGTAATTTATC
AAGATCGTACATTATTAACTCCAGCGATAAATGCTATTAATGCTTATGTTCCTGATATTC
CATGGACTAGCACATTTGAAAACTATTTGTCATATGTAAGAGAAGTTGCTCTAGCGGTAG
GAAGCGATAAGTTCGTATTTGTATGGCAAGACATCATGGGAGTTAACATGATGGACTATG
ATATGATGATAAATCAAGAACCATATCCAATGATTGTAGGTGAGCCAACTTTAATAGGTC
AATTCGTCCAAGAATTGAAATATCCATTAGCATATGATTTTGTTTGGTTGACTAAATCAA
ACCCGCATAAACGTGATCCAATGAAAAATGCTACTATCTATGCTCATTCGTTTTTAGATT
CTTCACTGCCAATGATTACTACAGGAAAGGGTGAAAACTCTATTGTAGTGTCAAGGTCAG
GTGCTTATTCTGAAATGACTTATAGAAATGGATATGAAGAAGCTATTCGTCTTCAGACTA
TGGCGCAATATGATGGCTATGCTAAATGTTCTACTGTCGGTAATTTTAACTTGACTCCTG
GCGTTAAAATTATTTTTAATGATAGTAAAAACCAATTTAAAACAGAATTTTACGTTGATG
AAGTTATTCACGAATTATCAAATAATAATTCCGTAACTCATCTTTACATGTTCACTAATG
CAACGAAACTGGAAACAATCGACCCAGTTAAGGTTAAAAATGAATTTAAAACTGATACTA
CCACTGAAGAAAGTAGTTCTTCCGATAAGTAATAAAGAAGTTTCTATTCCTAAGATGGGT
CTTAAACATTATAACATTTTAAAAGATGTTAAAGGTCCTGATGAAAATTTAAAGCTTCTT
ATTGATTCTATTTGTCCAAATTTATCACCGGCAGAAGTTGATTTCGTTTCTATTCATTTG
CTAGAATTTAATGGAAAGATTAAATCTCGTAAAGAAATAGATGGTTATACTTATGATATT
AATGACGTTTATGTATGCCAAAGATTGGAATTTCAATACCAAGGAAATACATTTTATTTT
AGACCTCCTGGAAAATTTGAACAATTTTTAACTGTAAGCGATATGTTATCCAAATGCTTG
CTTAAGATCAATGATGAAGTTAAAGAAATTAATTTTCTTGAGATGCCAGCATTTGTTTTA
AAATGGGCAAATGATATTTCTACAACTTTAGCAATTCCTGGCCCTAATGGTCCAATAACC
GGAATTGGCAATATTATTGGATTATTTGAATGAAAAAGCCACAAGAAATGCAAACGATGC
GTAGAAAAGTTATTTCAGATAATAAACCAACACAGGAAGCGGCTAAATCCGCTTCTAATA
CTTTATCTGGTCTTAATAACATTTCTACAAAATTAGATGATGCTCAAGCTGCTTCTGAAT
TAATAGCTCAGACTGTTGAAGAAAAATCAAATGAAATAGTTGGAGCAATTGATAACGTTG
AAAGCGCAGTAAGTGATACTACTGCCGGTTCTGAGTTAATTGCCGAGACTGTTGAAATTG
GCAATAATATCAATAAAGAAATAGGCGAATCACTCGGAAGCAAATTAGATAAATTAACGA
GTTTATTAGAACAAAAAATTCAAACAGCTGGAATTCAACAGACTGGAACGAGTTTAGCAG
TAGTCGAAAATGCTATTCCTGTTAAAGTCGTCGAGGATGATACAGCTGAATCTGTGGGTC
CTTTATTACCGGCTCCTGAAGCAGTTAATAATGATCCTGATGCTGATTTTTTCCCTACTC
CTCAGCCAATTGAACCAAAACAAGAGTCTCCAGAAGAGAAGCAAAAAAGAGACGCATTTA
ACTTAAAATTATCTCAAGCTTTAGATAAATTAACAAAGACAGTTGATTTCGGATTTAAGA
AATCTATTTCAATTACTGATAAAATATCTAGCATGCTATTTAAGTATACCGTCAGTGCTG
CTATTGAAGCTGCTAAAATGGTTGCATTGATAATGGCTGTTGTTATTGGAATAGACCTAC
TGATGGTTCATTTTAAATACTGGTCAGATAAATTTTCAAAAGCCTGGGATTTATTTAGCA
CTGATTTTAAAACTTTCTCAAGCGAAACAGGAACTTGGGGTCCTTTATTACAAAGCATCT
TTGAATCTATTGATGAAATTAAAAAGTTCTGGGAAGCTGGAGATTGGGGCGGATTAACAG
TAGCTATTGTTGAAGGGCTTGGAAGTGTTCTTTATAATTTAGGAGAGCTTATTCAATTAG
GTATGGCTAAATTATCTGCAGCAATTCTTCGAGTCATTCCTGGCATGAAGGATACTGCTG
ATGAAGTAGAAGGAAGAGCATTAGAAAATTTCCAAAATTCTACTGGAGCATCTCTTAATA
AAGAAGACCAAGAAAAAGTAGCAAATTATCAAGATAAACGAATGAATGATGATCTTGGCC
CAATAGCAAAAGGATTAGACAAAATTGCGAACTGGAAAACTCGTGCATCTAACTGGATTC
GTGGTGTAGATAATAAAGAAGCATTGACTACTGACGAAGAACGTGCAGCAGAAGAAGAAA
AATTAAAACAACTTTCACCGGAAGAAAGAAAAAATGCTTTAATGAAAGCTAATGAAGCTC
GTGCTGCAATGATTCGTTTTGAAAAATACGCTGATTCAGCTGATATGAGCAAAGACTCGA
CAGTTAAATCAATTGAAGCTGCTCATGAAGACCTTAAAAAACGAATGAATGATCCGGATT
TAAATAATTCACCAGCAGTTAAAAAAGAACTTGCTTCTAGATTTGCTAAAATTGATGCCA
CTTATCAAGAGCTCAAGAAAAATCAACCTGAAGCAAAACCTGAAACTTCTGCTAAATCAC
CAGAAGCAAAACAGGTCCAGGTGATTGAAAAGAACAAAGCACAACAAGCTCCTGTTCAAC
AAGCATCTCCTTCAATCAATAATACTAATAATGTTATTAAGAAAAATACTGTCGTTCATA
ATATGACACCTGTCACAAGCACAACTGCTCCTGGTGTATTTGGTGCGACGGGAGTTAATT
AAGGAATAATATGGCAATTGTTAAAGAAATAACTGCTGATTTAATTAAAAAGTCCGGTGA
AACAATTTCAGCCGGACAGAGTACTAAATCAGAAGTAGGAACTAAAACATACACAGCCCA
GTTTCCAACTGGGCGTGCTAGTGGTAATGACACTACAGGGGACTTCCAGGTAACAGATCT
ATATAAGAATGGATTATTATTTACTGCATACAATATGTCATCTAGGGATTCTGGAAGTCT
TAGAACGATGAGATCTAACTACTCTTCTTCATCTTCGAGTATTTTACGTACAGCCAGAAA
CACTATCAGTAATACAGTATCAAAACTATCAAATGGACTAATATCAGATAATAATTCAGG
AACAATAAGTAAAGTTCCTGTTGCAAATATTCTTTTACCTAGATCTAAATCTGACGTCGA
TACGTCATCACATAGATTTAATGATGTTCAAGATAGTCTTATTACAAAAGGCGGAGGAAC
TGCTACGGGCGTATTAAGTAATATGGCTTCAACTGCAGTATTTGGTGCGCTTGAAAGTAT
AACACAAGGAATAATGGCTGATAATAATGAACAGATCTATACAACAGCTAGAAGTATGTA
TGGCGGTGCTGAAAATAGAACAAAAGTGTTCACCTGGGATTTGACTCCGCGCTCAACAGA
AGATTTAATGGCTATTATTAATATCTATCAATATTTTAACTATTTTTCTTACGGTGAAAC
TGGTAAATCTCAATATGCTGCTGAAATAAAAGGATATTTAGATGAGTGGTATCGTTCTAC
ATTTATTGAACCATTAACGCCAGAAGACGCGATTAAAAACAAGACGTTATTTGAAAAAAT
GACATCGAGTTTAACGAACGTTCTTGTAGTTTCAAACCCGACAATTTGGATGGTGAAAAA
CTTTGGTGCAACTTCTAAATTTGATGGAAAGACTGAAGTATTCGGTCCATGCCAAATACA
GAGTATAAGATTTGATAAAACACCAAATGGTAACTTTAATGGATTAGCTATTGCTCCTAA
CCTTCCAAGTTCTTTTACACTCGAGATTACTATGAGAGAGATTATTACGTTAAACCGTGC
TTCTTTATATACGGGGACTTTCTAATGTATTCTTTAGAGGAATTTAATAATCAAGCAATA
AACGCAGACTTCCAACGTAATAATATGTTCAGCTGTGTTTTTGCTACAACTCCATCAACT
AAAAGCTCATCGTTAATAAGCTCAATTAGTAACTTTGCTTATAATAACTTGGGTCTAAAT
TCAGATTGGCTGGGATTAACTCAAGGTGATATTAATCAGGGAATTACCACGTTAATTACC
GCTGGCACACAAAAACTAATAAGAAAATCGGGAGTCAGTAAATATCTTATTGGCGCTATG
AGTCAGCGTACTGTCCAGAGTTTATTAGGCTCATTTACAGTTGGTACATATTTAATTGAC
TTCTTTAACATGGCATATAACTCATCTGGATTGATGATATATTCTGTAAAAATGCCAGAA
AATAGATTATCTTATGAAACTGACTGGAACTATAACTCTCCTAATATTCGTATAACTGGA
AGAGAATTAGACCCTTTGGTTATTTCATTCAGAATGGATTCAGAAGCTTGTAACTATCGT
GCAATGCAAGACTGGGTTAACGCTGTTCAAGACCCAGTAACTGGATTGCGTGCTTTACCA
CAAGATGTCGAGGCAGATATTCAGGTTAATCTTCATTCTCGCAATGGATTACCACATACT
GCGGTGATGTTCACTGGCTGTATTCCAATATCTGTGAGTGCTCCTGAGTTATCATATGAT
GGAGATAACCAAATAACTACATTTGATGTTACTTTCGCTTATAGAGTCATGCAGGCTGGA
GCAGTTGATAGACAAGCTGCCCTTGAATGGCTTGAATCTGCTACTATAAATGGTATTCAA
AGCGTTCTCGGAAATAGTGGAGGTGTTACTGGACTATCTAATTCGCTTTCACGACTTAGT
AGATTAGGGGGAACTGCAGGAAGCATTTCAAATATTAATACTATGACAGGAATTGTCAAT
TCGCAAAGTAAAATATTAGGAGCAATATAACAATGGGGACCGAAAGGTCCCCATATTTTT
ATTTACGGAATGAAATAAAAGCAGCAACTGAAGCAACAGAGGTTTCTTCAATATAAACCT
CAATTTTTACCGGAGCTTCTGACTCAAATTTACCTGTTACTACACCTTGGAAAATACTTT
CAGTCTGTTCTGGCTTTGAAAAATTTTCAGAAGGAAAAATTCCGAACTTTTTATCTGTTC
CAAAAATTTTAATAAATTCATCGTAAACTGCTTCATTAAAAGCATTATCAGCAGGAATAA
CGCCTTCAACTACAAGTTCTTGACCTAAAAAGCGCAAAGAAGATTTCATTTTGTGTTCCT
CATGTTATGTCAGTAAGACTACTATAACACAACATAAGGGACTTGTAAACTACATTTTGA
ACTTTTTAGACATTCCGTTGATGTTAACCAAAGAAGCCAAGATTTGAGAAACTTCGTTTT
CCTCTTCTTTTGATTCTTCTACAAGAAATTGATTAAATCCAGCCATTGGACGAACTTCAC
CAGTTTCCATTAAATGGTCGCCATCATATACCGATTCCTCTAATTGTTCAGAAGGAGGAA
CGATTGTACCTTCGATTAGATACTTGTTATAAGCAGTTTCTTTCATAAAGTACGTACTTA
CTTTATTAATCTTCAGTAAAATTCCACGCGGTAGAATAACTTCAGCTTCTGATGGAAATC
CTGATAAATGACCTGGAACGATAACTTTGATTCGCTCAGCTCCTTTAATTACCATACCCA
CGCTAGTTTCAGCATCTTCATTAGCATATGTAGAATGACTACCCATATGCATCAAATCTT
CTGCGTCAACTGAACCTTCTCCTTCACCAGAAAATACTGCGCCGGAATCATCTAGAGCCA
TATAGTTTTTACCATGCTCACCGAAGATATTTGGTTTAAGAGATGTGGATACGAAGTTCT
TGAAATAGAACATTTTGTTTTCAATGTTATGACGCAAAGTTTTAAAGGTAACTTCTTGTC
CGCGATAAAGCGTAGTTCCTGGAGGAAGAACTGAACCTTTAGCGAATGCAGAATCCAACG
TCTCGATGTGCTTAATAGCTCGTTCCATATATTCTTCACGGGTAGAATCTGGTTTACCCA
AAAGGAACGAGTTCATTTCAACATATGAATCTGCACAATAATCTTCAATTACATCAGATT
CGGCCTGAGTCAATCCTTTTGGAGTTACTTCAGTGCTTGCGTATTCATATGCCTGGGCAG
CAGATTCATTAATCAATTTATAAATGCCATTCATGACAAATTGAATAATAGCTGTCTTTT
CATCTGGCGTAGCTTTAGAACCTTTAACTAAATCATTAATAATTTTCTTAATAACATCAA
AGCTATTAGAAATATTAATGTCAGCAAGATCGCTTAAAATTTTATCAGTTTTATAATCAA
AGAATTCTTTATTCTTTAAATATTCTTGAGAACCTTGATATGTTATATCGCTCTGCAACT
TGATAGATGGATCACGTAAACGTGCATGGGCCAATGCAATATGATTTCGGAAGTTTTCTA
GTTCATAATCGCTAGATTTAAAACGAGAAGCACTCCTCATACCACCGATAGCTCCTTTGC
TGAAGTTTGAACCAATCTGGATAACGCCAGAGTTCATCGGACCTTCTGCTTTATATACCG
GAACTTTAGATTCAAACTCTTGATATTTCTTAGCAGCGAAGCTATCACCCTGAAGTGAAG
CATCGACAGTAGAATATTGAGCTTGTGCTGCTAATCGACGACTAATTTTAGTTTTTTGAA
TAACAGCTTGATCCGTGCGCTTATCATTTTCTAAAGCAATAGAAGCAGCAACAGCGACAG
CTTTAGGAACAGCATTACCTGTTTTAACATCTACATAAACATCACCGACCTTCGATTCAA
CTTTTGTGAATAACTCAGTTGAAATAGGAGGTACACCTGGAATGTCTTCTAAATTGGCAT
TTTTACGGTAAACCATAATATATGCATACTTTTTATCATAGTCCCAGATTTCCTTTAAAA
GAACATATCGACCTGCTCCTTTAGTACGTACTAAACGGTCAGCAATAACTTGCATTTGAC
GAGCAGCGCCATTTGTTTTACTTTTTAGTACGCGGAATAGGCATGCGTCAATTTTATACT
GTTTCATTGTTTGAAGAGCAGTATCAAAAACAGCATTAATGACGCCAATTGGATTTCCAC
CCAAGTTTCTAAGCTTAACCAGCGATAATTTCTCGTTGACACCTATCATTACGATATGCA
TCATTTTATCGCCTGGCTTAACGTTTTTATTCGTATCACCGCCAGAGGTGTATGTACAAA
ATCTAAATCCAGGAAGCACGCTTTCTTCACCGGCTTGAATAGCAAAAATTTGTGGTATTT
TATGCTTAGGATTTAAGTTAACAACCGGCGCCGAAGCGCCGTCAAATAATTCTGTAATAA
GTTCCATGATTTATCCTTGAACGAACTTGTAAGGCATGTTTGTAATATCTATGCAAGACG
CAATAATTCCAAGAGATGATTCTACTTTCTGTTTAACATTTGATGAAACAAATGATGCAA
AGTCAACTCTGTCTTCTATTTCAATATCACCTGTCATTGCAACTACTTCGCCGGTTTCCA
TTAAACGGTCACCGTCATAAATTACCGATTCGGTGAGTTCTTCGGTGGTCATAACTTCAG
CTTGAATGAGTTTTGTATTATTAGACGTGGTTCCGTCATTATTAGAAGCATCAGTTATTT
TATTAACTTTGACCATCAATCCGCGCGGTAGAATAACTTCGGCTTCTGTTGCTATTCCGA
GACTTCCTGGATATACAACATTAACTTTATGAGCTCCATCAATTGCCCATCCTATTTGAA
CTTTAACGTATTCAGGATTTTCTTTATACGCTCTTATTTCGTTTGGATTAATAGTTATTC
CTTCTTCATTTTTATCAACTGTTAATTCATTGCGAGCTTCTGGTTCTAAAAGACCAATAC
CAGCATGTGTAATTCCAAAACGTCCAAAAATGATAGGAGTTAAAGAAGTAGATACAAAAT
TTCTGAAATAGAACACTTTATTTTTAACTAGTGCTTCGTATATAGGAGCAGTCATACTTT
GAGCACGATAAACTGTAATGCCTTCCGGTATGCGGTCACCATTTTCAAAAGCTGAATCTA
AATTATTTATAGCAGATTCAATTTCTTTTTCACTCATTACATCATATCGTTCTGGATTGT
AACGACCTAAAAGAAGATTATTAATGTCTGTATATCCTGAACCACAATATTCTCTAATAC
CTCGCTTTTGTGATGGAGTATATTTAGAACCATCACGGGCAATAGTAATATCAATCATTG
GACCAGCACCAATTCCAGAAAAGCTATAAATCATTCGTTTGAGGAATTTAGTTTTTGCTG
CATTCCATGCATTTTTAGTTATTTCTCCCCAGACTTCATCTGGTTGCCCTTTACGAGTTT
TATTATGTTTATCAAGCCAATTATCTTTATATTCTTCAAAGAATTTATTAATAGCTGCTG
CGTATTTCTCTAAACATTTTATTGAAGTAGTTGGTTCATTTTTGATTGCGTTAAAAATTA
CATCTTTAATTTTTTCTGAAACTTCATCAACCTCACTTTTTGAAAAGAATTTATAGTCAT
AGTGAAGTTCATTCGCAGTTCCTTCGGCAATAATTTTTGCTGCCTTAGTTTTAGAAGCTA
ACGAATTAATTCCAAGCTTCATTTCTTCTGCTTCGGGAATTAAAGGAGCGGTAGCAGGTT
TATTGAAATTAGCTGCGGTAGATTCATACTTCTGGAATAATTCACTTTCAAATCTAGAAG
ATTCCAATGATTGACTTTGCGCAATTGCTCTACGTGAAATTTTAACTTTAACGATTACAG
CTTGGTCAGAACGTTTATCATTTTCTTGCGCAATAGATGCTGCAATTGCCTCATTTTTAG
TTACTTGAGCTCCAGTATCTTTATTGATATAAACATCACCGACCTTCGATTCAACTTTAG
TAAAGAGCTCGGTACTAATTTCCGGAACTCCTGGAATGTCTTCTAGTGATACATTTTTGC
GATGTATAAGAATATATGCATACTTTTTATCGTAATCCCAGAGTTCCTTAAGAAGGACGT
ATCTGCCACCTGAACGACTGCGGATAAGTCTATCAGCAATAAGTTGAATTTGACGAGCTT
GACCAGCAGTTTTAGACTTAAGAATACGGAGCATGCAGGCATCAATTTTATACTGACGCA
TCGTTTGCATTGCAACAGTAAAAACTGAATTGATGTAATTAATTGGGCTTGGACCGAGAC
CTTTTAATTTAGCAATTGAACCTTTAGCAGTTAATGTAAAAGGAACAATATGCATCATTT
TATCGCCCATCTTTAAATCGCGATTAGTATCACCTCCAGATGTATAGGTACATAAACGAA
AGCCTGGTTGTTCAATTGCGTCATCAACATGAACTGAAAAAATTTGCGGTATTTTCTTCT
TTGGATTTAAGTTTGTAATCGGAAGAGTAGTATCTTCGTCAAATAATTCTGTAATGAGTT
CCATCATATCCTCTCTAGTGTTTATTCTATTTATAAAATTAAAGGCCCGAAGGCCTTTAG
TAATCTAGTGGTAAGAGAGTACGATATATTTCAAACTTTGGACCTTTTTCATAGGCATCA
AATGTTTCTGTGAATTTATTATAAGCATATGCATCTACAAATTCGAGAATGACTTGTGAT
ACAGAAGTAGAAACATCTCCACCTTCTTTTTGAGCAATGACAATTGTTTCTAAGTAAGCT
TTCATAGACCAGTTACCTCATGAAAATCACCAAATACATCCTCGAATGTATTAGCTTTAG
TTTTATCTTCACGTAAACGAATCGCAATCGGAAGAAATAATTTAACGTAATCAGTACGTC
CATCAGATTTTAACCAACCGTTGCATTCGCACTCTAGAATTTTTCCAATATAATAATTTT
GGTTTTCCATAATGCGAGTACGATCAAGTTCATGCGATTTTACACCAGCTTTATCTTTTA
AGCCTGAACCAGCATTTACTTTAATTTTTCCACACTCTGATTCAAGAATAAATCCACCTG
CTTTAGTAGGGTCTTTACGGTGAGGATAAATTCCTACAATTTTTAAATCAACATCAATTA
CTTCTTTAAATTTATAGAGATTTTTTGAACGAGCATTTTCCCACAATCCATCGGTATTTT
TGAGAATAATACCTTCAAGACCTTGGTCAATATACTTTTTATAAATTACCTTAGCTTCAT
CTAGGTTATTTACTACCTGGTTTTCAATTAAAATTACTTTATCATATCCTGATGTCATTT
GTTCTAGTTTAGAAAAACGTACATCATATTTTAAACGAAACGCCGGAAGACCGTATATTT
CTACCAACGGTACATAATCCCAGACCTGAAACTTCATGCATTGTGCTTCTTTTTCAGAAA
TGGTTCCCTTTAAAGATTTATTGGCGATGCCATTAGATGCAGTACGAGATTCAGCTACTT
CGGCGAATTCTTTAGCTTTACTAATTTCAGGATAAGCATCAAAAAGAAAATCTAGGCCTT
CTGGCTCCTTTTTAACTTGCTCATGGTATACCAATTCGCCATCAATTAACACACCTTCTG
GATGAATCTGGCGGGCTTCAGCGGTCATTTTGATTAACTCTTCTTTGAGAAGGTCTAATC
CTAGATATTCATTACCAGCTCGTGATAAAAGACGAACATCATCTAATTCATCACCTCTGA
CTTCGGCAAAACACCGAGCTCCATCAGCTTTTAATTGGGCAAAGGCTGGAAATTTGATAT
TCTTATTAATGCCTTTTTCATCATAAGAACTTGCCAGCATTTGAGGTTGTTCAGGAATTA
AACCTGGCCAAACTTTGTTTGCAATAGATACTGAAGCACCGCATTCAAGGTCTCGCATCA
TCACCCGACGCAAAACTTCAACATCATCCTTTTTACCATCGGTGATATATCCAGTTAATT
CTTCAATTGCCGCATTTCCAGTCAATTTCCGAGTAGCTAATGTGAATTCAATGAAGTCAA
GCATATCGGTAAGAGTCAACATTCCAAAACTCTGGGTAGCAATACCAGGTTTAGGCCATT
TCTTGATATAATACTGTAACCCACGAGAATAAGTTAGACGATATACTCGTTTAAGCAATT
CATTATCTTTATTCTTTTCAAGAATTGCTTGCTTCTGCTTAGTTGAACCAATAGATGCTA
TTTCGTTCAGAATTTTAAGAATCATTGTTCATCCTTTAGAGTTTGGTTTACAGCTCTATT
ATAAATCAATTCATCATTAAGCTCAGTCAAAGACCTATGATATGTAGTTCTAACTTTATT
TCCTTGCATCCAGTGCTTGATATAAATGAAACCTTGCTCTACACATTTTTTAAAAATTCG
TTCGTCTTTTTGAGCTCGGAATTCTGGATTGCATCTAAAAAATTGATTTACGTGACCATA
ATCACGTGTAGTATTACCTTCATTTTCATAAATAGTGTGAACGACAAACATTAGAATGCT
CCTTGGAAAATATTATCACCACTAGTAAATCTATTAAAATACCCTGCATCACCAGGCTTA
GTATAACTCCACGTATGGAATGGATTCATATGATACGGGTGTGTATAATATGGATTATAT
GGATGTGTATAATATGGATTATACGGATTAAATCCAGGAGTTCTCCAGGTAGTGTTCCAA
CGAAAAGAATCGTTTTTAACCATCTTCTCAATTACATCTTTTATAGCTTTTTCACTATCA
AAACTTTCTTTATTTTCCTTCGGTGAAAAAAGCTTATTTTCTACATCATTCCATGTATAA
ACTCGCTGCGCAGTTTTTGGAATATTGTCACGCTCTCCTCGAGCCATCCAATAAACCGGA
ACATTTAATATTTCACTCGCATGATCGCAGTGGTGAGCAAGATCGTCAATATAACAAATT
ACGTTATATTTCTCTTTTGCCTTTTTGAATAACTCTTCTTTTGAAGAATCATGACCACAC
ATCATTACTTCTGAGAAGGCACCAGGAAAAAGAGCATTCAAATTAAATTGACGATTCAAC
AGAGCGTCAATAGAATCACCCAATGCTGTAACAGCTACAAAATTATAATCTTCTTTTAAT
TTGTTAATTACACACAGAGCATCTTTATACGGAGACAAGTAACGAATAAAATCTGAACGA
TTGTATTTTTCAATTAACTTGACGCCAAGTTCTTCATCACAATTAAATAGTTTACCTGGA
GAAATAAATTTCTCATCTTGGATCATTTTTAAAATATGTTCTAACGGAAGATTATATTTC
TGAGCAAAATAAGGAAGACCTGATTGCCAGCTTAAACATACTCCATCAATATCAGTTAAA
ATAGTAGGCTTCATAGAGAGTCTCTTAATAGGTTTAACACATCAATAAATTCAGCTTCGG
TTAGTATTGTATCATCTTTTGTTAGACCACTAGCCATGCTGTGCTTCAAAACTTTTCCTT
TCGAGGCTTGTAATGCATCGCGAAAGCCCCTATTTTGGATCGCTGCTTCAAAATATGCAT
TTGTGTATAATTCTTTCCACGCTGGGGAGTATCTTGAAAATGGAACTCCAAGCCAGAAGA
GGGTCCCACGGTCCTGAGCTCTAGCATAAGACCTTCCAGCTTGTTGGGCGGCAAGCCCGG
ATAACCCAAATATACGACGTTGTTGTTCAACATTTTTCACCTTGCACCCTTGGAGGAATC
CTTCAAGACCTCCAAATTGAATACCATCCATAACGAAAGGCCATTGGGCGAAATTACTTA
ATGCACATGATGGCCACCTAAAATTGCTTCTAATCTCTAACTCAGACATTTTCAATGCTT
ATAATTTCAACATCAGCCCAATGACCATAGCAAGGAAGACGAAATTCAACTGGCCAGTTA
GGGTCCTTTTCTAATATAATAGACTCAACTCGTGGTTCTTCGTGTTCATCAGTATATGGA
TTTTTATCTGTTACTTTATATGTGACCTTAATGTACTGAATTCCAAAAATCTTATTAATT
ATATTCATACTAATTCCTTTAATCCGTAAATAGGAGATAATTCATCGCCCATACGAAGGT
CTTCATTTCCATCTACCCAGGAAACAATATAAGCCTCTTTTATTTGAATACCACTCCATT
TAAATGGAGGTAGAACCTTAGAAATTAATCCAGGTATACCAACTCCCTTTAATTCAACAG
TTTGACCTAAAAAGAATTTCATTAGAACCTCATCTGAAAACCGTGCGATTTAACATTACC
GCCGCCAATATCAGAGATATTAATTTCGCGCGCAATTGAAGGGTCAATGTCAATTTCACG
ATTGAGTTTAGTGATAGCTAAAGTATCGCGCCCATTTACAGTACGGAACTCTAAAGGACA
TACTACATTAACGTAGTTTTTAATCGATTCGCTGGTAGGTTGCAAATGAGCAGGAAGGTC
TTTAGGCGCCTTAGAGAAAACCACTTCACAGAAGTTTTTACGAGTATCAAAATAAGTAGT
CATAAACATAGTATTTTCCTCAGCAAGGGGCCGAAGCCCCTCATTTTATTTTAAATATCA
AATTCGTTAAGAACTACATCAAAGATTGCTTCAAGATGCTCAGGTTTAGCTCTATTACTC
AGAATATGACGAATCCAAGTTTTAACCAAAAGCTTGCGATTAGCACCATTCCAGCAAGGA
TGAGTCCCTAAATCGCGTTCACGGAAATCATCATCCAGAGCGATTTTAAAGTTGGAACCT
TCCATCGTGATTGAAACCGTGATACCGTTTTCAAATCGCATATAAACGTAATTAGGAGTC
ATATACTGCTCAATTTCGCATACTGATCCATTTTGATGTTTCCAAAGGCAAATAGTATCA
ATAGAACCTGCAATACCATTAGAAACATATTTACGTTCAAAGTTGATGTAGTTCATTTTA
TTCTCCAAGATGTTTAATTGCGGTACAGGTATATAATATCATATCCTGTACCAAAGTAAA
CTATTTTATGCAACTTTAATCCAATTATCTAAATTTAATTCACCTTTGGCTTTCATTTTT
TCAATCAAGAGGTCAGCGGTAATACGACCATTTTGATACCACGTAACATCTTCAGAGTAA
CCAGTTTCTTTATCTTCATAAGTCTCACAACGAATTTTACCAATAATCAAAGTTTTCAAT
TCGTATGTATCTCCAGTAAAAAAGTTAGTTAACATTATTTTCCAAGAGAAATCATCAGAA
GGACCTTCAGGAGTCATGCAACTTACAAATTCAAATTGTTCTTTGTATAAGTGCCACTGA
AGTTCTTTAGTTTCGATAGCTTTATAGCCGTCGTGATCTTTACCTTTGATGTATGAGTTA
AGATTGATTTTCATTTTATTCTCCTTTGTTTGTGTAAGATAATACTATCACAAAGGAACT
ATACTGTAAACAACTTTATGCAATCTTTGGAAAATAAAAAAGGACTCCCGAAGGAGTCCT
CAACTTATGCTTTCTGCTTACCAAAACGAGAAGCATCATCTCGAAGAACCGCACGTGCTC
GGCGCATGATCTTCTCAACAGTTTGATTGATACGAGAGTTCGACCCACGCTTGTAGCCAG
CGCGTTTAGAATCACCAACTTTCTTTTCAACTGCTTTCTTTGCTTTAACTTGTTTTGCCA
TTATAAATTCTCTTTTAAATGAAAATGCAGGACTTATTGGCATTGCCTGCGCAAGCCCTC
AAGGGGGTACTTAGGTTTTGTATATTTACCGACAGGATAACCATAAACCCGTCATCATTC
ACATTCAAGAGGTACACCGTAAAACTGCCGGGGTCTTAAAACTATAATGATTCGCAAATC
ATTAATCAGACAGTTCGACGGCTCCTCGATTTTAGCTCACACTAAGGCAGTGAATCTCCA
ATAAATTACTTCAGTGTTACCACAAAGTGACGAACTGCTTTTCGTGCAGCAGAAGCCAGA
GGCTTAGCATATTTAAGTTCATCTTTTTCCTGAAGCTCAGCAGCTAATGCAGTTTGAGCA
GGATTCAGATGTTTAAAATAACGCAGGATTTCAAGAGCTTCGGCTTCAACATCAATAGAT
GCGCCATAGTTTTCGTGACCATTATTCCATGCGTTTCGTTGCAGTTCAAGAGCGTGTTGT
AATTGTTTAATCATTTAAAAATTCTCGTTAGAGATTAAAACTCGGTAGTCACGTTCTTCT
GAATTTCATCTTCTTTCGACAGATCTCTCAGTTGTAGACTACCACATAGAATTGTTCGGT
TAACTTATTATTCCGACACCCAATTCATATTATTATTTATATCACTTATAAAGACACGGA
ATAGCTTTATAGTGACAGGTAACGAATTTTTGCTTAATTTCTTTTGGCTGCTTAAGACCC
AGAGCTACAAAAGGATGTGGAACGTTTCGAATTTGACCAACTGGAAGAGAAGTCAAATCA
CCAACTTCACAGAAACCTTCAGGAACATCAGGACCAACAGAGTGAATTACACACAGTTCA
GGAACTTCACCTTGAACACGTTTACCGATAATAAGTCCTGATTCTGTAACTTCTTCATCA
CCGGCTTGTGCAGGTTCAGAAACTAAAATAACATATTCACCGACAGCACGAATTGGTAGC
TGTTGTACTTCAGACATCGTTTTTCCTTTTTGTTAACAGATGAATTAATAATAACAAATA
ATTCTTAAAGCATTTATTTACCAATAAATTGAAGCAAATGCTCAACTTTCATACCATTAA
CGGAAATCAATTTGTCAATAGAAAAACCTCGCCACGCACCAAGCTCAACATCAAATACTG
GAATCATGTCAGTAGATTCTTTCCGAGTAGATTCAGTCAATTTACCAGTTTGCATGGTTG
GCATAAAGTCTGCATCACGAGTACCTTTCATAGTACGAATAGTACCGTCAGACTTTTCAA
AAACTACGTTTGAAACACCCATGGACAGTTTAGTTTTCAAAATTTCACGAATTGCTACTT
TCTGCTCAGTTGTCAGTTTCATTTATTTACCTATTACAGTTTTAATATGAGTTGTTCCAC
GTTCTTTAAGGGTGGAAAGTAATTTTTGGCATTTTTCTAAATCAGATTTCCAACTATACG
GTCTATCAATACTAGTCCAATCAGTTTTATAATACTGTTTCCATTTAGAGAAAAAATATT
TCTTATACTCTACAGCAAATGAGATGTTCTCGTTAGAATAAGAACTAATTGCTGTAAGCT
TTACCAAACGAAATTTCATTATTCACCACAGAATTCGTTGATATTTTCCCAATTTAACTT
ATTCAAGTTTTTCTTAGGAACATTAAACACTTCAATACCTGCATTTCGCAGAATATCATC
CCAGCCAGGTTTATTTTTATCATATGTTTCGCAATAAACCAGCTTTTTAATACCAGATTG
AGCTATTGCTTTTGCGCAATCTGGGCAAGGAGAAAGTGTTACATACATAGTAGCACCTTC
AATAGAAGAACCATTTCGTGCAGCAAACAAAATTGCATTTAGTTCAGCATGAATTTCATT
TTTAGACGACCATTCAGAGTGAGCGCTACGATGTTCTTTCGCCAAAACAAAACGATCAGT
TGAACCAAATGATACGCATTCAGGCTTATGACCTTGAATGATAGTGTGTTTAGGCTTATT
CAACAACCAACCTTGCTCAGCAGCATAATCACAACAGTTCACACCCCCTGCTGGGGAACC
ATTATACCCAGTAGAAATAATACGTCCATTCTTTTCAATTACTGCTCCTACCTTCCAGGA
GCAACATTTTGATTCCTGTGATACTAAATATGCAATTTGAAGTACTGTACTCGCTTTCAT
TTCATAATCACCAGATAAGCAGATTTAGCAGTTTCAACACGATAAATTTCGTGACGAAGT
TTAGTTATACTTTTAATAACAGAACTAATTATATTCTGCCCATCTTTAAAGCGGTTTTTC
TTATCAATAAAAATTGCGCCAACCATCTTTTTGTGAAGCTCAACTGGATACTTCGTCACA
ATAATAGCATCATACACAGAAGGATGAATACTATTCACCAGAGTATCATTCATTAAAGTT
ATTCTAATGAACTGTGCTGTTTCAGAATCAAGCGCTCTATGATCGCCAGCATCATTTTCA
AGACAATTATCAATTATATCAGTTAAATTCATCATAGTACGCCATACACCCTTTGTGCTT
CAACTAATCCATCAAAATCCAATTTAAGATGCGATATTTGATCGCCATCACCTGGATTCA
CAATTACTAATACTGAACGAGGAGTTTCGGTAATAACACGAACCGATGTTTCAGGAAATC
GTTCAGAAACCTTATTTACTAATTCCTGCGCAAATAGTTTAACTTTTTCTTGGAATTCTT
TAACAGTAATTGGTTTTTCACTTAGCAGCATTTTGTTTTCCTCATTTGTTTGGTAGAGCT
ATAATATCACAACTCTACCGTAAAGTAAACTATTAAATTGCTTTGAATTCTGCTGTCTGC
GATTCAAAACGAATGTCGCCTTTGATAACTAACTCAGCATCAAGACCGAATACAACAATG
ATATGCGCGGAACCTGGATATAGCGTAATTGCAATAGAATCCACCTGGTCTGGAAGCAAA
GTGTTCAATACATGAGTCACTTGAGCATGAATTCGAAGCTCGGCTGCGTTATCAAGCTTT
TCAAACATATTATTAGCAATAATTTGGCTAAACACTACTTCTACGATTTTAGAATAAGTC
GGAAACATATTTACCTCACATAATTTTCTTCAAGCCAATCAATAACATCCAATGCATTAT
CAAAAGTTGAACCATCTACTTTGTCTTCTGTTTCATAATCAAGAACATCTAGGCCTAATC
TTCCGTTAACAATAGGCCATAGACAAAATAGATATTTCTTTTCTTTTTCAATTTTATCAC
AAAGACGATAAATCTTTTCTATGTTATTCATAAGTTTTCCATGGTAAAGGCAGTTTAGTT
TTCTTTACTGTTAGTTCAATATCAGGATTCTTTTCTCTTAATTTAAGGCATTCCTCCCAA
GCTCTGATTTCACTAGTGAATACACAAAATTGCCCATTACTAGTACCGACTAAACCACTA
TTTACAATAATAATAGCCCAAGTTTCATGATGCCACGCCATTAAAAATCTCCCGAAGCAA
CTTGCCAGCATTCAACACCGATACGACGCCACATCTCAACTACTTGAGTTCGGTCATCAA
TAGCTAATTTCACATCAAAATGTGGTGCGATGTGTTTCCAGAAAATTTCTTCTTTAACTA
CATCATCTTTACGGGTATCACCTTGTTCGCGTTGACACTGCATAACTAATGGAACGCCAG
CAATATCCTCAACCCATTTACGAGTCATACGATAATATTTCGTTGGGTCTTCTTCGGTTC
CACTTTCACGGCCTGAAACGACTACGATGTAATATCCTTGCTTATCGTACATCTTAGCTA
GTTCAATAACCATAGGATTGACAACATCAGTATCGCATTTTTCAAGGTCATAAGGACCAC
GACCATTCATTTTTGCTAATGTGCCATCAACATCAAAAATAACTGCTTTTGGTTTACCAG
GAGTCCCATTATATACTGGAAGACCGAGATACTCTCGCATGCTTTTATACATTGAGCGTA
AAACATCAATTGGTACTGCTTTAGTTCCGCGTTTTGAGTTACGTTTAACCAATTCAGTCC
AAGGAACATCAAACACTTTATGTTCAACTTTCCAGCCGTATTCTTTGGCAAAAGTTTCCC
ATGCTAGGCGACGTTCAGGATTCAAGTTAGTATCTGAAATGATTACGCCCTTAACAGAAT
CGCCACCGTACAGAATACTTTTAGCTGTATCAAACTGCATACCAGTTACGATGCCTTCTT
TCTTTTTGGTATACTTATACTCGTCGCGTTCTTCATGCGCCATAATAGATTGGCGATAGT
CATCACGATTGATATTATAAAACCCGGGATTCTTAGCAATAAATTCACGAGCCCAAGTAC
TCTTACCAGAACCAGGACAGCCAATAGTCAAAATAATCTTTTTCATCATTTAATTCCTAA
GAAAACTTCAAGAATACGAATATTATGCTCACGCCGTCCTTTGTATAATTCAGCTGTTGA
TTGATTCACTGATTTACTCTGAACATTGGATGAAATAAATTTTATCATGTGATTTTTAAG
CTGATGCATATCAAGCCCACGGGCTTTTGCTTCTTTTCGTAATGCTTTACCAGCATCATC
TAAAGCTTTAGCATGTTCAATGTCTTCGCTAATGCTTGATACATTTTCACGGCATACGTC
AATGTAACGGTCTGAACATTCAATATAACGTTCTAGCAGAGTTTTCATTTATTTTTCCTC
AACCAATGATTGAATATAATCATACAGGTCTTTAGATGCTTTACCCCACTTATTTTGATA
TTCATTTTTGAGATTAGCACGTGATTGAGCTAATAAAACATCATTAGTTGGAGGTAAAGA
TTCTAACCGCTGAATCTGGCGTCCATAAATCATTGCAGCCATCTCGGATTCATAAATCAA
TCCTTTGAGATGTTCAAATTGATGCCATGAAATCATTTACATTTATCCTCTTTTAACTCT
TGACGATAATAGCATATCATAGTTTTCTGGTCATGTACATATCGTTTTACATCATTAAGC
CAAATACGAAATTCCTGAGAATCTTCAAATGGCATACCGACCCAAGCTTTACCATCGATA
ACTTTAACTTGCCAAGATAGTTTAGCTTCGTCATATGACTTTATTTGTACAGGCCAATTA
GGATGAACTGTTTCTTTCTTTACTTCTAGAGGCTTTGTAGAACAACCAACTAGAAGACCA
ATAGATAATATTACTGCTGATAGTTTAATCATTTAGAAAGGTCCTGGATGTCTTCTGCGA
ACTTGTTGAAGGAGTTGTTGATTTGTTTTTCAACCAATCCTGGCTTATGAGCCACCACAT
CCGCCTTCTTTGCGTCTTTGCGCAGTTTTTCATTTTCACGCTCAATAGCAGCAATTGCCT
CACGATTTTTATTATTCATCGCATCAATATAATTATACTGAATTCGCAAATTATTTAATG
CTAAGGCGTTTTCATTGGCCGTTTTTGTAATTTCAGTAACCGATGTTTCTAATCTTTCTA
CCTTATTTTTTAAAATAATAGATGTTCCGCCAAATACTATTACAAGTAATAGCAAACCTG
CTGTCGTATTACTTAATTGCATAAAGTTTTAATAACCTCTATAATATCGTCTTGAGAAAG
ACCGTTAATTAAAATATGATGTTCAGCAGGAGATTTAGAAATTTTAAAGCATGCCTCAAC
ATCTTCTGCCATATCCGATGCGCTACGATTTGGATTACTAATTCCAAGACGATGTTTTCC
CGTTAAAGGATTAACGATGATATAGCATTTACAGTTGTTAATATGAACATTAGGTTGAGT
CTGATTAATGAACACATCACAATCATACTTAGCGAGTTGATTTTCTAAAAAGACTTTCAT
CTCTTCAACCGCATCAGGAAGCATATCACGGGCTTGCTCAAGACGACGATTTCGATATTC
TTTAATGGTCGTTTTCCGCTTGACTTGCTTAGCTAAATCTTTCTTAAGATCGGTGATATA
TCCAACTCGACGATTTCCTTTGAATACAGAAATCCCATCTGTAGTATCACCGTATGCTTC
AACGACCATTTCAGTAGTAATAAGCTGCAAATCCATCATAAAGTCCTCATGTTATGTCAG
TAAGACTACTATAACACAACATGAGGGACTTGTAAACAGCTTAGTATCCTTCTGGGATAA
ATTTTTTATAATTTTTCAAAAAATTCTGTTCGATTTCACACATAACCTTTTCTTGACTAT
CGTACCCCTGGTATAGGCTCATAATGATACCGAACAGGTGATCCATTCCAGCACCTTTAG
CAACACCTTGCGCTTCCATCGCATAAGTCTTTCTATCCTTACCACAATGCTTATTATGAC
AGTCAAGAACTAAAAACAGAGCTCGGTCTAAGTACTTCAGATAAGTCGTTTCAAATGCTT
CAATTTTTCTGTATGAATATTCATCGTCAGCGTACATTGCTTTAAGATCATCTGATGCAC
CATCAATAATAGTCTTAAACAATTTTTCTGGATTATCTAATGAACTTTTTGTGCTATGAA
GAGACACGTACCAGTCAGACTTAATTTTAAAATGAGAACCATCTTTCATTACAGCAACAT
AGCCTTCGATGTTTTCTGCATTTTTAGCTTCTTCTACCCATTTAGGGCTATCAATTTCGA
ATCGTTCAACTAGATATGGACGAAGAGCAGCATCTTTATAAATATCATCGTATGAAATGT
ATTCACCCGTTTCGTTTTCACGAATATTCAATAAAATGATTTTCATCTCTTGATAAGCAA
GAACGATTCTATTAGTTGGAGCGACGAATTCGAAGTTAGCAGTAAATCCATCTTCGGCTA
ATTCTTTAAGTCTGTCGCGCAACGGGTGATGATTAATATTCATCAAAATTCCATTAGCCA
TTAAAGCCTGTTCGGATTTGATTGAACCCTTTGATTTGAACAGAATTTCATCACCGTCTA
AATAAGTTGATACCAAAGACCCGTCTTCTTTTGTTAGAATGTAATCAACATCATTTAAAT
CGATATTCATCGTGAATGGGTTTTCATTCAAGTTAAAAAACTTTTCCATAGGACGAGAGG
CGATTCTTACTGGTTTTTCTCCATCCATTTCAAACATAATTCCACGACATTCTAATGCGT
CTGGAAGTAACCAATCAGAATAAGATGCATAATTATATGAGAAAATTCTGTAAGTTCTTC
CAGATGCACTTACATCATCCGAGTAAAAAAACTTACGCTGTGAATCCTTACACAGTTCCA
TTAAATTGTTAAAAAGTTCTTGCATTGTGTATCCTCTTTTGTGTTTTGAATATAGTACCA
CACTCCATGTGGAAGCATCATTTTTTCTTATGTTGAATATTCCAAGGCGGGTTAAATAGC
TTAATGAATAGTGGCTCCTCTAGGTCAATCGTTGCGATTGTCATTGTACCTAACTCATTT
GTCATAGAAAGATTAAAACATTGGCGGGCGTAAAATTCAACTTTGCTTCCTTCCTTTAGC
GCAGCATGAATTAATGCAGATTTAGTAGAATCAGACGTTTTGTCTTTACGGTTAATAGCA
GTTCTATAATAGTTTATTCTTTTACGTAAATTTTTAGTTTTTCCAATATAAACAAGCTCA
TCATTTATAGCAATAGCATAAATTACGTTATACTTGTTTGGAATAGATAATTGTTTTATA
CTTCCGTTGTCGTCTAATTCTAGTTCAGTATATTTAATAAATGAATATTCTGTTGCAATT
TCTTTCATAATAAAATGGGCCTTGCGGCCCACTCCTTAAAAGTATTTTTTAAAACTCATC
ATAACTTTATCATCAACATCATTATCAATCTGTGCAACAAGATAAGATGACAGTTCTACT
TCTTGTGGCGCGGATTGAACATTATCAGAATTAAGATATTCACGAATCCAAGGATATGGA
TGTTTAACCGGAGCATCGGTAATTGGGCATGGAAGACCACACTGTTTCATACGAGATACA
GTTAAGTAATCAATAAAGCTCCACATGCTATTTGTATTTAATCCAGGAACATCGCCATCT
TTAAATAAATGAACTGCCCAATCTTTTTCTTGGCGGTTAACTTCCATGAAAATATAAACT
GCTTCTTGTTCACACTCTTGAGCAATTTTAACCCATTCATCGCCATCAGTACCAGATTGA
AGTTGACGAATAATATATTGTGTACCTTTAAGATGAAGCTGCTCATCACGTGCAATGAAC
TTCATAATCTTGGCATTACCTTCCATGATTTCCATGTTCTTATGGAAGTTAAAGGTACAT
GCAAAAGATACATAAAAACGAATAGCTTCTAAGGCATTAATAACGTGCAAACAGAGGTAA
AGAGATTTCATTAGCTCACGCTTCCAATATGTCTCATGCTCTATGGCATCTTTAATATCA
TCTTCGTCTGCGTTAATTTCTTTTTGGTATTCGATATCAGCTTTAGCGTTTTCCCAATAA
CGAGTTTTAGTCAGAACATCATCATAATAACGTCCAATGGATTCAGCACGTTTCATAATA
GCTTCATCTAATACAATTTCATCAAATACCTTCGATGGATCAGTATAAAGATTTCGCATG
ATATGAGTATATGAACGGCTGTGAATAGTTTCACTAAAAGTCCATGTAGCAACCCATGTG
TCAAGACTTGGGTCTGAAATTAACGACATAAGTACAGCAGACGGCGCACGACCCTGAATG
CTATCCAAAAGTGATTGATACTTCAGGTTATTAGTAAAAATATTTTGCTGGTACTGAGGA
AGCTTATTAAATTGTGCAGCATCCATCATCAAGTTTACTTCTTCAGGACGCCAAAAAAAT
GATAAAGATTTCTCAATAAGTTCTTCAAATACTCTATGTCGTTGAATATCATATCGAGCT
AAACCCAATCCAGAACCAAAAAACATCGGCTCAGTCAAAATATTAACAGGGGATGTATTA
AAAACTGTACTCATTTAGAATCCTTAAATTTACATTTATCATAATGCCATCTTAAAGCAT
TACCTTTATTAACTTTCTTGCCACAATGCGGACAAGGCGGATACTCTGTACCTTGCTTAG
TTCTCAAAGAAATCAAATCACGGGTTTCTTTAGTATGAGGAACATCTCTTGTCGGAGAGA
TTGTACCATACATCGGATTAAGCACGCCGACTTTAGCAGCAGCTATATTCTTTTTAGCTT
CTTCGGTTTTAGGTTTTCTCATGCGAGCTAAATGCTCTTTGGTAAATTTCCTTCCTTTAA
GAGCTCTACTTATCTTTTCTCTAGTTTCCATAGAAACTTCACGTCCTGTAGCAGCTTCAG
ATAAACGTCGTCTACCTAATTCGTACAGTTGAGAAGAAATTTTATATGAGCGATTTTTAC
CAGTCAAGCACATTAAACCAAAAGCTTTCTTTAATCCTGGGGTGTCATATATTTTACATA
AAAGCCAATGAGCTATAAAATGCTCTCTACCTGTTAATTCAACTAAATTTTCTTTATCAT
CAGAACCTCCCATACATCTAGGGATTATATGATGAGTCTCTTTATATCCTAATAAAGGTT
CCCGGGTTTGAGCCCGGGAAATTAAACTATTATATACGTTTTGATAATTCATTACAATTT
ACAGGATGAACAATCTTCAGCTTTTGGAGTTTCTATTTCATAATCATCAGTACCGGAACC
ATCACGGGTATTATGATAATAGAAATTTTTTCCGCCAAAATACCAAAAATACAAAAGGTC
ATCAATCATTATTGACATTGGAACCTTGCCTTTAGGGAAGATTTGGGGGTCATAGTATGT
ATTCGCCGAAGCTGATTGACAAACCCATTTCAGCATAATAGCTACCTGCGTAAGATAAGG
TTTATTACCTTTCTTAGCTAATTTCCATGTATAGTCGTAAAGGTCTATGTTATGCTCAAT
ATTGGGCACGACCTGATTAAAGGAGCCCTCTTTTGATTCTTTAACAGAGACTGGTCCACG
TGGAGGCTCGATACCGTTTGTACTGTTAGAAACTTGGGAAGATGACTCACATGGCATAAG
TGCTGATAATGTACTATTACGGATGCCAAAGAGCTTAAGGTCTTCCCGCAGCGCCGACCA
GTCACAAACGTATTTTGGAGCTGCGATTTGGTCAATCTTTTTATTGTACCAGTCGATAGG
TAATTCGCCTCGAGACCAACGAGTGTCTGAATAATATTCGCAAGGTCCTTTTTCTTTGGC
GAGCTTAATGGATGCTTTAATGAGTCCATACTGTAATCTCTCAAATAGTTCATGTGTTAA
ATCGTTAGCATCTTCATAAGAAGCAAAGTTACTTGCCAGCCAAGCTGCATAGTTAGTAAC
ACCTACACCAAGGTTTCGACGCTTTTTAGCTTTTTCTGCTTCAGGAACTGGATATCCTTG
GTAATCCAAAAGATTATCAAGAGCACGAACTTGAACTTCTGCCAATTCATTAATTTTATC
TTGGTCTTGCCAATCGAAGTTATCCAACACAAATGCAGAGAGAGTACACAACCCAATTTC
AGCATCAGGGCTATTCACATCATTTGTTGGAATAGCAATTTCACAGCACAAGTTACTTTG
ACGAATAGGTGCCTTTTCACGAATAAACGGAGTATAGTTATTCGTATTATCAATGAACTG
TACATAAATCCTTGCTGTTCCTGAACGTTCAGTCATGAGCAATTCAAATAGTTCACGGGC
TTTAATACGCTTTTTACGAATATTAGGGTCTTTTTCTGCTGCTTCGTATAATTCACGGAA
ACGGTCTTGGTCTTTAAAATAAGAATAATAAAGCTCGCCACCCATTTCATGCGGACTGAA
CAAAGTAATGTAATCGTTCTTTCCAAATCGTTCCATCATCAAATCATTCAACTGAACACC
ATAATCCATATGACGGATGCGGTTTTCTTCTACGCCTTTGTTATTTTTCAAAACGAGAAG
ATTTTCAACTTCCAAATGCCAAATAGGATAATAAGCAGTAGCAGCGCCGCCACGAATTCC
ACCCTGTGAACATGATTTAACTGCAGTCTGAAAATGTTTCCAAAAAGGAATAACACCGGT
ATGGCGTACTTCACCCATGCCAATCTTAGAACCTTCGGCACGAATCATACCAACATTAAT
ACCAATTCCAGCGCGTTTAGAGATATATTCAACAATTGAAGCGGAAGCCTTATTGATAGA
CTTCAACGAATCTCCTGCCTCAATAACAACACATGAACTGAACTGTCGAGTCGGAGTACG
ACAACCAGCCATAATAGGAGTTGGCAGTGAAATCTGTCGAGTAGATACTGCTTCATAAAA
ACGGATAACATGTTTTAATCTATCAACAGGTTCATCTTGATGCAGTGCCATTCCAATAGC
CATAAATGCAAACTGTGGAGTTTCATAAATTTGACCAGTGGTTTTATCTTTAACTAGATA
TTTTTCTTTTAATTGCATCGCCCCGGAATAAGTAAATTCCATATCCCGCTCGTGCTTAAT
TTTTGATTCTAAAAATGTAATTTCTTCTGCTGAATATTTTGACAATAATTCAGGGTCATA
TTTACCTTCATTTACACAGTAAGAAATATGGTCAATAAATGAACGCGGTTCATACTGCCC
ATAAACATGCTTACGAAGAGCGAACATTAAACAACGTGCAGCTACATATTGATAATCAGG
CTCTTCAACCGAAATAGAATTCGCAGCAGCCTTAATGACAATAGTCTGAATATCATCAGT
TGTCATTCCATCACGGAGATAAGATTTAATATTTTCATATAATTCATAAGGATCGACTGA
TGTTCCTTCAGCTGCCCAAGATAAAACTTTAATAATTTTTTGTGGGTCAAAGCTCTGAGA
AACACCACTACTTTTGATAACATTAATTAATTGCATAAGTCCTCAACTTGAAAATCGTCT
TTAAACAATCGGTTAACTATATGAGCTATTATATCACCATGACACGGCTTTGGTTTACAT
GTGCATCCTAGCCTCATTCCACGTAAAGGCTCTAAATGTGCTTTAGTTATTTCTCCGGAT
TTAATTCGACGTATAAAATCTTTTTTGAATAATTCAATGGCAGCCTCCCGGCTGCCAGCA
TCTTTACCGACGTAATTTCCCCAAAATGTACCACGGTGAATATTAACATCAAAGTCGGAT
TTGTATTTATTCACTACCCGGCATAGACGGCCCACGCTGGAATAATTCGTCATATTGTTT
TTCCGTTAATACAGTAATATCATAGTAACAGTCGGAAGAAGTTTTAACTGTGGAAATTTT
ATTGTCAAAATACTCACGAGTCATTTTATGAGTATAGTATTTTTTACCATAAATGGTAAT
AGGCTGTTCTGGTCCTGGAACTTCTAACTCGCTTGGGTTAGGAAGTGTAAAAAGAACGGC
ACCAGAAGTATCCTTAAATCGTAAAATCATATATCCTCTCAATTAAATTAAAATTATACC
GCCATCTTTCCTTTAATTGACGGATGAGATACATAGTTGTTAAGAACAAAATTATGCGGC
TTTGCGAATTGACTCAACCAAAATACCTGCGATTCTGTGCCCCATTCATCAAATTCATCT
GGAAATTTTAATTGTAATTCGCATAAATCCATAGGTTCACGACGCAAAATTTCTTTACAT
TGTTCTACGTGATTCATATAGATATGAGTATTACCACCAGAAAATATCAAATCTCCCGGA
ATAAGATTACACATCTTAGCTACAATATGAACTAACGTAGCATATGATGCAATATTAAAT
GGAAGACCGAGGAAAACATCTACCGAACGTTGATACCACTGCAAATCTAAATAGCCATTA
CGCACATTAAACTGATAGAACATATGACAAGGCGGTAATGCCATATATTTAAGTTCAGCT
GGATTCCACGCAGAAACAATTTGACGCCTATCATTTGGCAGTTTTTTAATACGATCAACA
ACTTCTATAATTTGGTCTACACCGCCAAAATCACGCCACTGTTTTCCATAAATTGGACCA
AGTTCACCGCTATGGTATCCTAAATCTTTTGCCTGATTTTCGTAATTTTCATCCCAAACT
GTTTTGCCTTGGATTAACGAATCGTGTTGAATTAATCGTAAATCATTAACATTTGTGCTT
CCTGATAAAAACCATATTAGCTCAGCAATGCATGCATTCCAGGCGAGCTTCTTAGTTGTT
ACTGCAGGAAAACCTTTAGTTAAATCCCATCGTAATTTAGTACCGAACAGAGCAATTGTT
CCTGTGCCCGTTCGGTCATCGGTTTCATAGCCATTTTCAAAAATGTCTTTAATTAAATCT
TGGTATTGTTTCATTTAGTACCTTATATAAATTATAATACATTTTATGAAGCTGAATAAT
CAATACGGTTTAGCATCAAATTTTCTCTCAATTTCAGGCGTGAAATATAATTCTTTACCG
CGTTTGGAAAAGCGACCAATAATATGATAATTATTCTCATCAAACGTGATATTACCAATA
TCCATAGCTTTCAACCAAATATCGATATTTCTATCCAGCTTTATATCAAAATTCCAATAC
GGAGAACTATTACCGAAAATAATGCAAGCGGATTCAGTGAAACAGCTACTATAAATCTTG
TCTTTAGGAACTACTAATTGATAGACACCTTTAATAAACTGAGGACCTTGCCATTTTGGC
TGTTTCTCGTCTTTTTCTAACAGAATAGGGTAATAATTAAAATTCATATTTCCGCCACGC
GTTATTTCATTTATATACTGATTCCGTAAGGGTTGTTACTTCATCTATTTTATACCAATG
AGTTTCAACCATTTCACGCTTGCTTATATCATCAAGAAAACTTGTGTCTAATTGAACCGT
TGAATTAACACGATGCCTTTTAACGATGCGAGAAACAACTACTTCATCTGCATAAGGTAA
TGCAGCATATAACAAAGCAGGCCCGCCAATTACACTTACTTTAGAATTCTGATCAAGCAT
AGTCTCGAATGGTGCGTTAGGGCTTGACACTTGAATTTCGCCGCCAGAAATGTAATCTAT
ATATTGCTCCCAAGTAATATAGAAATGTGCTAAATCGCCGTCTTTAGTTACAGGATAATC
ACGCGCAAGGTCACACACTACAATATGGCTACGACCAGGAAGTAATGTAGATAATGACTG
GAACGTTTTAGCACCCATAATCATAATTGTACCTTCGGTACGAGCTTTAAAATTCTGGAG
GTCCTTTTTAACTCGTCCCCATGGTAAACCATCACCTAAACCAAATGCTAATTCGTCAAA
GCCCTCAACCGTTTTAGTTGGAGAATAAGCGAATACCAATTTAATCATTTTAATTCCTCA
ATATAAAATATGTCAGACGGTTCAATATAATTGCTCAAGCGCTGACCATGATAATTATAA
ATGCAAAAGTAAAATTGGTTTTTAAATTTATTTCGCACTAAAGAAACCGTATAGTAATCA
CCGTTTGAGGTTTGAACGACAAACAAATTATCATAATAAATTAGATTTTGGTAGATATCA
GCGATATCTATGTCTTCCCCAACCTTAAGCAAATTTAGTGTTTTCACTATATTTCCTCAA
ACAGACTTTTTCACAATTTTCCAATCAGCTTTAAACTGCTCGACGTCAGAATGGTAAATC
CAAAATCCTGCGCTTTCTCCGTCTTCATAAAGAGGACATCCATCGCATTCATCTTCCCAT
CCCATGTCACGTAAAAGATGTTCAGCTTTTTCAACGAGATCAGAATCTTTACCGATGATA
TTAAAATACCATTTACCTCTAACTTCTGAATCTTTGATGCTCTGGCGTTGTAATCTCATT
TTATTCTCCTCAGTAGTTGATAGGGTAATAGTATCACACTACTACCCTGTTGTAAACTAC
TTTTTGAAAGTTTTTCGCAAAAGTTCAATGATTTCATCTACATTGTTTTCGTCAACAATG
CAGTGAATTTTTGTTACGCCAGAAACTATACCTTCATTAGCGGATTGAGGCTTTTCTAAA
GTTACCTCAGTAAAGTATTTGAATTCTTCAGAGTAAATCTCAAAATACGTCTCAAGTGGG
CCACCCGCCATGTATTTAGTGCCATCTTCCATAGTTACACGTACCACACATTCACCAGCA
CAATCATCACTTTCCATTTCATCTACAACAAAACTAGGTCCGTGCTTCTCTATTAAATTT
AGCATCATTACATTGCAACGGCACTCGTTAGCAAAGTCATTTTTAAATAAAGGGTTAATG
ATATAAGTTTTTCCAACTTCCATTTTGATTTCCTCAGTTTAACGGGGCTATCAAATAGCC
CCTTGATAATTATTGTTCAATCAGTCCCATGTAAAATTCTGCGTCTTCAGAATCCATACC
ATCACAATACTCATTAGCCATAAAGCGGGTGAGGTCTTCAAGAGGACCTTCAATAACGAT
TTGAATACTCCAAAACTTAGAATCTTGCACGCTTGTGATACTAAGTTCAGGATAACGATT
ACGAATAATTTCTTCGATATATTCAAAATCAACGATGTCAATATCAACTTTAGCCATATT
ATTTTCCTCTTTAATCATCAGCAGTATTGCCGATAGTTGTATAGTACCATGGAAGGACAA
GGATGTAAACCGTTTTGTGAAAAAATTTTTGAAATAAAAAAGGGGACCCTAGAGGTCCCC
AATTAATTAGTAATATAATCTATTAAAGGTCATTCAAAAGGTCATCCAGGTCCGTGTCAT
CAGCACTAGATGAACTGCCAGAGCTTGAGCTCATAAAATCATCTTCAGTTTTTGTTTTGA
AGTCATCAACATTAAATGCATCCAAATCATCAGCAACTTTATCGGCTTTCTTAGCAGCAG
TTGCTGCGGCACCACCCATAGCAGCAGTTCCCATAACTTGACTGAACTTAGTGCTCAGTT
CTTCAAACGATTTGAATTTATCTTTAGAAGTCATTTCAGAAAGGTCAACCATTTGTTCGA
ACAGTTCTTTCTGGAAAGATTCATCATCAATGTTTGGAATCGCAGATTGATTCAGGAATT
TAGATTCGTCGTAGTTACTAAAACCAGAAACTTGTTTAACTTTCAGTACAAAGTTAGCAC
CTTCCCACGGACAAGTTACATCAACCGGAGTTTCACCCATTTCAACATCAACTGCAATCA
TTGCATTGATTTTATCCCAGATTTTCTTACCGAAACGGTATTTAAATACTTTACCTTCAT
TTTCTGGAGCAGCTGGGTCTTTTACAACAAGAATGTTAGCCCAGTAAGAAGTTTTACGTT
TAACAAGACCGTATTCTTTATTGTCAGTGTTGTACAAATCATTTTTATTGATGTACTGAC
ATACTGGACAAGAATCGTAATCACCGTGGGTAGATGAACAGTTTTCAATATACCATTTAC
CGTTTTTCTTGAAACCGTGATTTACAAGAATTGCGAATGGTGCTTGTTCATCATTTTTAG
ACGGAAGAAAACGAATTACTGCTTGACCGTTACCCGCATTGTCGAGTTTCAGTTTCCACT
CGCCTTTATCTTCAGAAGAAAAACCACCTTTATTTCCAGCCAGTTTAGCCATTTGTGCAG
CGAGTTCAGCAGTAGATTTACGTTTAAACATTTTTATTTCCTTTTTAATTTAATTAACAG
TTGGTGCTATGACGATGTATGACCTCATAGCTGGTCAGTGAGATAATTATAATCTATTTA
TAATAAGCAATTAATACTTACAAGATTTCACAGTTTCAATAAAAACACTTTTAGCTTTCT
GTGAATCAATATTTAAAATTTTTCTATAAGCCTTTAACTTTATAGAATAATTATTCCAGA
CTAAATTATCAGTCTGTTCATCATGTTTATCGATTATATTTAAAAACGAATCAAGCAAGA
TAAACGTCTCAAACGAAATTATATTCGATTGAAGTAGTTTAAAAATATAACTTGATTGAA
CTTTTGGATTATACTCAAAGATTTCTTTAAAAGCAGAAACTTCAACTTTTTTACTAAAAT
AGTAAATATTGCGAATATCTTCTTCAAACTTAAATTTAATTTGCTTTAAGCGTCCGATAT
ATTCACGATAAAACACAAGTGCATCAGCGTCAGAGATGTCACCAATCCAAGCATCTTGGT
TAGCAACCAGATTGCTTATAAAGATTAAAGCAAGTTCCTTTAATTTATATTTTTCTGATA
ACTTCTGGAAAAAATACTTATCCCTTCGCTTTTGATAAGCGGCATCAGACACCCGCATGC
ACCAATTATACTTAATTACATCATACTTTCCATTCATATGTTGTTTTATCATTAAGTATA
ATTTATAAACTGATTTACCATCAATATATCTTTCACCACCAGCAGGCATGCGGAGTTTAA
TCATAATAGAAAATCTAATGTATTAGTTTTTTCACAACGAACAACAGAAGGACGTAAAAG
ATTTTCGTCAATAGCTTCTGACTGAATTTTTTCAATTATACCCGAAGGAATAAATTTAGC
AAATTGAGTTTCAGGAATAGAATTTTCTTCTAAGAATGCTGTTGTAGCTTCAAGATAACT
CATTCCAAACTCTTCTACCATTTTTTCAATAATAAATCCATTTTCTTGGCGGTCAAGAAG
CTTTGCAATTTCATCCTTTTCTTTCTTAATTGAAAGTTCTTTTTCTGAAAGACCGGTCTC
ATCGACCGGACGAATATCATTTAGAGAAAACTGTGTCATAAAGTTCAACTACCTCTTCAG
TTTCAGCTTCAAACACATCACGGTTATCTTTATGATACAAAGCTAACAGACGATTAAACA
TCTTACCATCAACGCCAAGTTCATCTTTGGCACGAATTCGAATATCTTTAATCAGTTCAT
TATAACCGGAAATTTTCAGTTTATGATCAGATGCTTCTTTAATAAATTTAGCCAAGTCTT
CGCCATGGATAGCTTCATCAAATTCAACCATTTCTTTTTTAGCCATTATTCACCTCAAAA
TTCATTAATGCTATTAGTTAATTTAGAAAGACCCGCTTTTACAAAATATGAATAAATTTT
GCCACGAGGTGGTAATTTATATGAATTATAGTAATTCACAATGTTTGAAGCAATATTATC
AGGAATATAATCAAAATCAATTAGAACTAAATTTTCTTTATAACGATTATATTCAGATTC
TGTGAGAAGCACCTTAGCTTGCTCACGGTCATTAGCAATAGCTTCAACGATTGAAGTTTT
CATTGAAGGAGTTCGTTCACCTTCAACTCTGGTAAACCAAAAGTCAGATCGTACTTTAAC
TGAAGCAACGTTATCCTTTTTGTCGCCTTTAAGGATTTTAGTCATACAGTCAATTTCAGC
AGAACCGCTTTTAATTTTAACCCATTTCTTGTGCATCGGTGACCATTGCTTAACATTTGG
GTATTTGTGAAGCTGTGTAAAGTCACCATCCGACGAAATGATTAAAATCTTATGTCCTTC
TAAAGAGAACTTTTTAACAAGAACAGCAATGTGGTCATCTGCTTCATACTTATCAATATC
CATAACAATGTATGGCATATAAGCTTTCAATTCATCTATAACTTTATGACTGGATTCAAA
ATAACCTTCCCAGTCCCAAGTAGATTCTTCTCGTGCTTTTCCACGGTTTTTCTTATAATA
ATAAGCAAAATCACGACGCCAGTATCCAGATTTCGCGTTATCAATACACAATATAATTTT
AGTGTATCCAAGAGTTTTTGCTTTTTTGACATTAAACTTAATTGAGTTCAATATCAAATG
ACGAACCATTGATAAATTAATTTTTTCTTTATCTGGAAAGTTTACCAAAGCAGTTGAAAG
CGCAATTTGACTAAAGTCAATTAAGCAGATTCCTTCTTTGTAATCTTCATCCAGCATCAT
TTCTAAATCCATATGAACCTCGTTCAATTAGTGAGATTTCTATTATATACCATCCGAATC
TTAAAGTAAACAAGTATAAATACTTATTATTGAAAACACAATAGGAGCCCGGGAGAATGG
CCGAGATTAAAAGAAAGTTCAGAGCAGAAGATGGTCTGGACGCAGGTGGCGATAAAATAA
TCAACGTAGCTTTAGCTGATCGTGCCGTAGGAACTGACGGTGTTAACGTTGATTACCTAA
TTCAAGAAAACACAGTTCAACAATACGATCCAACTCGTGGATATTTAAAAGATTTTGTAA
TCATTTATGATAACCGCTTTTGGGCTGCTATAAATGATATTCCAAAACCGGCAGGAGCTT
TTAATAGTGGACGCTGGAGAGCATTACGTACCGATGCTAACTGGATTACAGTCTCATCTG
GTTCATATCAATTAAAATCCGGTGAAGCTATTTCGGTTAACACTGCAGCTGGTAATGATA
TTACATTTACTTTACCATCTACTCCAATTGACGGTGATACTATCGTTCTCCAAGACATTG
GAGGAAAACCTGGAGTTAACCAAGTTTTAATTGTAGCTCCAGTGCAAAGTATTGTAAACT
TCAGAGGCGAGCAAGTACGTTCAGTACTAATGACTCATCCAAAGTCACAGTTAGTTTTAA
TTTTTAGTAATCGTCTGTGGCAAATGTATGTAGCTGATTATAGCAGAGAAGCTGTAGTTG
TAACACCAGCGAATACTTATCAAGCGCAATCCAACGATTTTATCGTACGTAGATTTACTT
CT GCTGCACCGATAAATATTAAACTTCCGAGATTTGCTAATCATGGCGATATTATTAATT
TCGTTGATTTAGATAAACTGAATCCACTTTATCATACAATTGTTACTACATACGATGAAA
CGACTTCAGTGCAAGAAGTTGGAACTCATTCCATTGAAGGCCGTACATCGATTGACGGTT
TCTTGATGTTTGATGATAATGAGAAATTGTGGAGATTGTTTGACGGGGATAGTAAAGCAC
GTTTACGCATTATAACGACTAATTCTAATATTCGTCCAAACGAAGAAGTTATGGTATTTG
GTGCGAATAACGGAACAACTCAAACAATTGAGCTTCAGCTTCCTACTAATATCTCCGTTG
GTGATACTGTTAAAATTTCCATGAATTACATGAGAAAAGGACAAACAGTTAAAATCAAAG
CTGCCGGTGAAGATAAAATTGCTTCTTCAGTTCAATTGCTGCAATTCCCAAAACGTTCAG
AATATCCGCCTGAAACTAAATGGGTAACTGTTCAAGAATTAGTTTTTAACGGTGAAACTA
ATTATGTTCCAGTTTTACAACTTGCTTATATAGAAGATTCTGATGGAAAATACTGGGTTG
TACAACAAAACGTTCCAACCGTAGAAAGAGTAGACTCTTTGAATGATTCTACTAGAGCAA
GATTAGGCGTAATTGCTTTAGCTACACAAGCTCAAGCTAATGCCGATTTAGAAAATTCTC
CACAAAAAGAATTAGCAATTACTCCAGAAACATTAGCTAACCGTACTGCTACAGAAACTC
GTAGAGGTATCGCAAGAATAGCAACAACTGCTCAAGTGAATCAGAACACCACATTCTCTT
TTGCTGATGATATTATCATCACTCCTAAAAAGCTGAATGAAAGAACTGCTACAGAAACTC
GCAGAGGTGTTGCTGAAATTGCTACGCAACAGGAAACTAATGCAGGTACTGATGATACTA
CAATCATCACTCCTAAAAAGCTTCAAGCTCGTCAGGGTTCTGAATCATTATCTGGTATTG
TAACCTTTGTATCTACCGCAGGAGCTACTCCAGCTTCTAGTCGTGAATTAAATGGTACAA
ACGTTTATAATAAAAACACTAATAATTTAGTTGTTTCGCCTAAAGCTCTGGATCAGTATA
AAGCTACTCCAACACAACAAGGTGCAGTAATTTTAGCAGTTGAAAGCGAAGTAATTGCTG
GACAAAGCCAAGAAGGATGGGCAAATGCGGTTGTAACGCCAGAAACGTTACATAAAAAGA
CATCAACTGATGGAAGAATTGGTTTAATTGAAATTGCTACGCAAAGTGAAGTTAATGCAG
GAACTGATTATACTCGTGCAGTCACTCCTAAAACTTTAAATGACCGTAGAGCAACTGAAA
GTTTAAGTGGTATAGCTGAAATTGCTACACAAGTTGAATTCGACGCAGGCGTCGACGATA
CTCGTATCTCTACACCATTAAAAATTAAAACTAGATTTAATAGTACTGATCGTACTTCTG
TTGTTGCTCTATCTGGATTAGTTGAATCAGGAACTCTCTGGGACCATTATACCCTTAATA
TTCTTGAAGCAAATGAGACACAGCGTGGTACACTTCGTGTAGCTACACAAGTTGAAGCTG
CTGCAGGAACATTAGATAATGTTTTAATAACTCCTAAAAAGCTTTTAGGTACTAAATCTA
CCGAATCGCAAGAAGGCGTTATTAAAGTTGCAACTCAGTCTGAAACTGTGACTGGAACGT
CAGCAAATACTGCTGTATCTCCAAAAAATTTAAAATGGATTGTGCAGAGTGAACCTACTT
GGGCAGCAACTACTGCGATAAGAGGCTTTGTTAAAACTTCATCTGGTTCAATTACATTCG
TTGGTAATGATACAGTTGGTTCAACACAGTCGTTAGAATTATACGAGAAAAATAGCTATG
CAGTATCACCATACGAATTAAACCGTGTACTCGCTAACTATTTGCCATTAAAAGCAAAGG
CTGCTGACGCAAATTTATTAGATGGTCTAGATTCATCTCAATTCATTCGTAGGGATATTG
CACAGACGGTTAATGGTTCACTAACCTTAACCCAACAAACGAATCTGAGTGCCCCTCTTG
TATCATCTAGTACTGCTACGTTTGGCGGATCAGTTTCGGCAAACAGCACATTAACTATTT
CTAATACTGGAACTGCAACTCGTCTGATTTTTGAGAAAGGACCTCAAACTGGAACTAACC
CAGCACAGACGATGACTATTAGAGTTTGGGGAAATCAGTTTGGCGGCGGTTCAGATACAA
CACGTTCTACTGTATTTGAAGTTGGTGATGAAACATCTAATCACTTTTATTCTCAACGTA
ATAAAGCTGGTAATATTACGTTTAGCATTAATGGTACTGTGATGCCAATAAATGTTAACG
CTTCCGGTTCATTGAATGCGAATGGCGTAGCAACATTTGGTAGTTCAGTTACTGCTAATG
GTGAATTCATCAGCAAGTCTGCAAATGCTTTCAGAGCAATAAACGGTGATTATGGATTCT
TTATTCGCAACGATGCTGCTAACACCTATTTCATGCTTACTGCATCGGGTGATCAGACTG
GCGGATTTAATGGATTACGTCCTTTAGCTATTAACAATCAGTCCGGTCAAGTAACGATTG
GTGAAAGCTTAATCATTGCCAAAGGTGCTACTATAAATTCAGGTGGTTTAACTGTTAACT
CGAGAATTCGTTCTCAGGGTACTAAAACCGCTGATTTATACACCCGTGCACCGACATCTG
ATACTGTAGGATTCTGGTCAATCGATATTAATGATTCAGCTACTTATAACCAATTCCCGG
GTTATTTTAAAATGGTTGAAAAAACTAATGAAGTGACTGGACTTCCATACTTAGAACGTG
GCGAAGAAGTTAAATCTCCTGGTACATTGACTCAGTTTGGTAACACACTTGATTCGCTTT
ACCAAGATTGGATTACTTATCCAACGACTCCAGAAGCACGTACCACTCGCTGGACACGTA
CATGGCAGAAAACCAAAAATTCTTGGTCGAGTTTTGTTCAGGTATTTGATGGAGGTAACC
CTCCTCAGCCGTCTGATATCGGTGCTTTGCCATCTGATAATGCTACAATGGGGAATCTTA
CTATTCGTGATTTCTTACGAATTGGTAATGTTCGCATTATTCCTGACCCAATGAATAAAT
CTGTTAAATTTGAATGGATTGAATAAGAGGTATTATGGAAAAATTTATGGCAGAGTTTGG
ACAAGGATATGTCCAAACGCCATTTTTATCGGAAAGTAATTCAGTAAGATACAAAATAAG
TATAGCGGGTTCTTGCCCGCTTTCTACTGCAGGACCATACGTTAAATTTCAAGATAATCC
TGTTGGAAGTCAAACATTTAGCGCAGGTCTTCATCTAAGAGTTTTTGACCCTTCCACCGG
AGCATTAGTTGATAGTAAGTCATATGCTTTTTCTACTTCAAATGATACTACATCAGCTGC
TTTTGTTAGTTTCATGAATTCTTTGGCGAATAATAGACTTGTTGCTATATTAACTAGCGG
AAAGGTTAATTTTCCTCCTGAAGTAGTATCTTGGTTAAGAACCGCAGGAACGTCTGCTTT
TCCATCTGATTCTTTATTGTCAAGATTTGACGTATCATATGCTGCCTTTTATACTTCTTC
TAAAAGAGCTATTGCATTAGAACATGTTAAACTAAGTAATAGAAAAAGCACAGATGATTA
TCAAACTATTTTAGATGTTGTATTTGACAGTTTAGAGGACGTTGGAGCCACAGGATTTCC
TAAAAGAACATACGAAAGCGTTGAGCAATTTATGTCGGCGGTTGGAGGAACTAATAACGA
AATTGCACGTCTACCAACTTCAGCTGCCATAAGTAAATTATCTGACTATAATTTAATTCC
TGGCGATGTTCTTTATCTCAAAGCACAGTTATATGCTGATGCCGATTTACTTGCGCTTGG
AACTACAAATATATCTATCCGTTTTTATAACGCATCGAATGGATATATTTCCTCGACACA
AGCTGAGTTTACCGGGCAAGCTGGTTCTTGGGAATTAAAAGAAGATTATGTGGTTGTTCC
AGAAAACGCTGTCGGATTTACGATATATGCACAGAGAACTGCGCAAGCTGGTCAAGGTGG
CATGAGAAATTTAAGCTTTTCTGAAGTGTCAAGAAATGGAGGTATTTCGAAACCTGCTGA
ATTTGGCGTCAATGGCATTCGTGTTAATTATATCTGTGAATCTGCTTCACCTCCGGATAT
AATGGTACTTCCTACGCAAGCATCGTCTAAAACTGGTAAAGTGTTTGGGCAAGAATTTAG
AGAAGTTTAAATTGAGGGAGCCTTCGGGTTCCCTTTTTCTTTATAAATACTATTAAAATA
AAGGGGCATACAATGGCTGATTTAAAAGTAGGTTCAACAACTGGAGGCTCTGTCATTTGG
CATCAAGGAAATTTTCCATTGAATCCAGCCGGTGACGATGTACTCTATAAATCATTTAAA
ATATATTCAGAATACAATAAACCACAAGCTGTTGATAACGATTTCGTTTCTAAAGCTAAT
GGTGGTACTTATGCATCAAAGGTAACATTTAATGCTGGTGTTCAAGTTCCGTATGCTGTA
AACAACACAAACCCATGCGGTGTTTATCCAGGTAACGGTGATAATGCTACTTTTGCTGTA
GCAAATATCGATATTGTTTCGTGGAATGGTATTGGTTTTAAATCATCACAAGGTTCAGCA
GCTCGAACTGTTGTAATTAATGCACGTAATGGTGATATTAGTACAAAAGGCGTTGTGTCG
GCAGCAGGTCAAGTAAGAAGTGATGCGGCTGCTCCTATAGCAGCGAATGACCTTACTAGA
AAGGACTATGTTGATGGAGCAATAAATACTGTTACGGCAAATGCAAACTCTAGGGTGCTA
CGGTCTGGTGACACCATGATAGGTAATTTAACAGCGCCAAACTTTTTCTCGCAGAATCCT
GCATCTCAACCTTCACACGTTCCACGATTTGACCAAATCGTAATTAAGGATTCTGTTCAA
GATTTCGGCTATTATTAAGAGGACTTATGGCTACTTTAAAACAAATACAATTTAAAAGAA
GCAAAACCGCAGGTGCACGTCCTGCTGCTTCAGTATTAGCCGAAGGTGAATTGGCTATAA
ACTTAAAAGATAGAACAATTTTTACTAAAGATGATTCAGGAAATATCATTGATTTAAGCA
TTTCCGCTGGCGGTAATATCAGTGGAAATGTAACTATTGATGGAACTTTACGCGTCAATG
GACCAATAAACAATTTTGGAAATTTTTCCACAAGTGGCCAGATTACCGCTGGTAGTAGTA
TTTCAGCTCAAGTTTTTAGAGCATTGCAAGGTTCGTTTTATACAAGAGCATCAAATGATG
CCTCAAATGCCCATTTATGGTTTGAAAATGCCGATGGCACTGAACGAGGCGTTATATATG
CTCGTCCTCAAACTACAACTGACGGTGAAATACGCCTTAGGGTTAGACAAGGAACAGGAA
GCACCATTAATAGCGAATTCTATTTCCGTTCTATAAACGGCGGTGAATTTCAAGCCAATC
GTATTTTAGCATCAGATTCATTAGTAACAAAACGCATTGCAGCTGATACAGTTATTCACG
GTGGCAAAGCCTTTGGTGTATATGATACGGATTCATTAGTTAACTACGTATACCCAGGCA
CCGGTGAAACTAACGGAGTAAACTATCTTCGTAAAGTTCGCGCTAAATCAGGCGGTACCA
TATGGCATGAGCTTTGTACTGCTCAATCAGGCCAAGCTGATGAAATGTCTTGGTGGACCG
GTTCTACTCCTATTTCAAAACAATTTGGTATTCGTAATGACGGTCGGATGATTGTCCGCA
ATAGTCTTGCATTAGGCACTATGACTACTGATTTTCCATCGAGCGATTATGGTAATGGTG
GCGCATTAGGATTCGGTGCTTATTTGGCTCTAGGTGATAACGGGACTGGTTTACGCTATC
AAAAAACCGGTGTTTATGATTTAGTTGGTTCCGGATTGTCTGTTGCATCTATTACTCCTG
ACAGTTTCCGTAGTACTCGTAAAGCATTATTTGGACGTTCAGAAGACCAAGGCACCACAT
GGATTATGCCTGGAACGAACGCTGCATTTTTATCAGTTCAAACACAGGCTGATGAAAACG
CTGCTGGAGACGGCCAGACGCATATTGGCTATAACTCAGGCGGAAAAATGAACCACTATT
TCCGTGGTAAAGGCAAAACTAATATTAATACTCAGGAAGGTATGGAAGTTAATCCGGGTA
TTTTGAAATTGGTAACTGGCTCTAATAATGTAATGTTTTATGCTGATGGCGGTATTACAT
CTGTTAAGCAGCTTTCAATATATAATGGCATATATTTAGCAGCTAATAATTCTACTGCTG
GGCTAACATTCGCTCCAACTACTACAATTGATGGTACTAAGCAAATTTTGTGGGAAGGCG
GTACTCGTGCTGGGCAGAATAAAAGCTATGTAACTGTTAAAGCGTGGGGTAATTCATTTA
ACGCATCTGGCGATCGTTCCCGTGAAACAGTTTTTGAGGTAGGTGATGGGCAAGGTTACC
ATTTTTATTCTCAACGTGTAGCTCCTGCGCCTGGTTCGACTGTCGGGCCTATTCAACTCC
GAGTTAATGGTGGTTTATTAACTTCAGGTAGTATTGTTGCTTCTGGTTCTATTACAACCG
AATCTTCTTTGAGTGTTAATAATGGCATGTCAGTAAACGGACAAGCTAAATTCGGTGGAA
CTGCTGATGCATTAAGAATTTGGAATGCCGAATACGGTGCTATTTTCCGTCGCTCAGAAA
CAGCATTACATATTATCCCGACTCCTAAAGATGCCGGTGAAAGTGGTGGAATAAGTAATC
TCCGCCCACTTAGTATTAGTCTTAATAATGGTATGGTACAAATGCGCCATTCTGTTACGT
TAGGCGATGATGGCGCTGGCGGTAATATGATTACTGTGGACAACGATACCAAACTTGTTG
TTGTTACTAGTAATAGTCGCATTTCTCCTAATTACCGTATGCAGGTAGGACAAGCAGCTT
ATATTGATGCTGAATGTACTGACACAGTTCGCCCGGCTGGTGCAGGGTCTTTTGCTTCAC
AAAATAATGAAAACGTTCGTGCTCCATTCTATATGAATATTAATCGCACAGATACAAGTA
CTTACGTTCCTATTTTGAAACAGCGATATGTCCAAGGAAACAGTTGTTATTCTCTGGGAA
CTCTAATTAACGGCGGTAACTTCAGAGTTCATTATCATGAAGGCGGCGATGGCGGTTCGA
CTGGCGCTATTATAAAGGATTTAGGTTGGGAATTTACCAAAAATGGTGATTTTATATCTC
CTAAGCGTGTTTCTGCTGGGGGTTCAGTTTATTTAGGTACAGACGGCAATATTACAGGTG
GTTCTGGCAATTTTGCTAATTTAAACAGTACAATTGAATCACTTAAAACCGATATTGTAT
CGAGTTACCCAATCGGTGCTCCTATTCCATGGCCAACGGATACTCCTCCTGACGGTTACG
CTATTATGGAAGGCCAGACTTTTGATAAGTCTGCATACCCAAAATTGGCTATGGCTTACC
CATCTGGCGTAATTCCAGATATGCGCGGGCAAACTATTAAAGGTAAACCAAGTGGTCGAG
CTGTATTAAGCGCAGAAGCCGATGGTGTTAAGGCTCATAGCCATAGTGCATCGGCTTCAA
GTACTGACTTGGGTACTAAAACTACGTCGAGTTTTGATTATGGTACTAAGACGACAAGTA
GCTTTGACTATGGCACTAAGACTAGCAATACCACAGGTAACCACTCTCATACTGTAAGCG
GTACTACGAGTTCTGCTGGTGCACACCAACATGCGCGCTCAGGTCCTCAGTTACAAGCCG
GCATACCCAATAACATATTCTATGACGGATATAATAGTGTAGGTGCAAACGCCAACGCTA
AAATCACCGGATCTGTGAGCGGTGCTACTGCAGGGTCGAATATAGCCAAAACTTCTTCAG
ACGGTGCTCATACCCATACTTGGTCTGGTACTACAAGCACTACAGGTAACCATGCTCACA
CAGTAGGTATTGGTGCTCATACCCATACTGTAGGTATCGGTGCTCATACCCACACGGTAG
CAATTGGCTCGCATGGTCATACTATCACTGTAAATAGTACAGGTAATACAGAAAACACGG
TTAAAAACATCGCTTTTAACTATATCGTTCGTTTAGCATAAGGAGAGGGGCTTCGGCCCC
TTCTGAATATGAAAATATATCATTATTATTTTGACACTAAAGAATTTTACAAAGAAGAAA
ATTACAAACCGGTTAAAGGACTCGGTCTTCCTGCTCATTCAACAATTAAAAAACCTTTAG
AACCTAAAGAAGGATACGCAGTTGTATTTGATGAACGTACTCAAGATTGGATTTATGAAG
AAGACCATCGCGGCAAACGAGCATGGACTTTTAATAAAGAAGAAATTTTTATAAGTGACA
TTGGAAGCCCGGTTGGTATAACCTTTGATGAGCCCGGCGAATTTGATATATGGACTGATG
ACGGTTGGAAAGAAGACGAAACATATAAACGCGTTTTAATTCGCAATAGAAAAATTGAAG
AATTATACAAAGAATTCCAAGTTTTAAATAATATGATTGAAGCTTCTGTCGCGAATAAAA
AGGAAAAATTCTATTATAAAAACCTTAAGCGGTTCTTTGCTCTTTTAGAAAAGCATGAGC
ATTTAGGTGGTGAATTCCCTTCATGGCCTGAAAAAGAACGGAAACCTTGGTATAAGCGTT
TATTCAAGCATTACGTATAAATATCTTAAAAGGAGGGTCTATGGCAGCACCTAGAATATC
ATTTTCGCCCTCTGATATTCTGTTTGGTGTTCTAGATCGCTTGTTCAAAGATAACGCTAC
CGGGAAAGTTCTTGCTTCCCGGGTAGCTGTCGTAATTCTTTTATTTATGATGGCGATTGT
TTGGTATAGGGGAGATAGTTTCTTTGAGTACTATAAGCAATCAAAGTATGAAACATACAG
TGAAATTATTGAAAAGGAAAGAAATGCACGCTTTGAATCTGTCGCCCTGGAACAACTCCA
GATAGTTCATATATCATCTGAGGCAGACTTTAGTGCGGTGTATTCTTTCCGCCCTAAAAA
CTTAAACTATTTTGTTGATATTATAGCATACGAAGGAAGATTACCTTCAACAATAACTGA
AAAATCACTTGGAGGATATCCTGTTGATAAAACTATGGATGAATATATAGTTCATTTAAA
TGGACGTCATTATTATTCTGACTCAAAATTTGCTTTTTTACCAACTAAAAAGCCTACTCC
CGAAATAAACTACATGTACAGTTGTCCATATTTTAATTTGGATAATATCTATGCTGGAAC
GATAACCATGTACTGGTATAGAAATGATCATATAAGTAATGACCGCCTTGAATCAATATG
CGCTCAGGCGGCCAGAATATTAGGAAGGGCTAAATAATTATTTGTTCGTATACATCTCTA
GATATCGATATACACCCTCAAAACCCTCGTTGAATTCGTCGATGAGGGTTTTCTTATCTT
CTTGAGTTAATTCAGAAACAATTTTACGGAATGAATTCTGATTTAATTTTCTACCTTCAT
GCGTTACTCCAATCTCATTCAGAAATGCAATAAAATTAGCACGATTCTCAACAATATCTT
CTCTGGAAAATTTAATCAAAATAGACGCAACGGTAATAATTTCACGAACTGTATCAATGT
TTTTATTCATTAACTATACCACTCAATTAGTTGACTTTGTTATAATATCATCAGACGCTT
GATTTGTAAACTGGTCTGTGTTATTTTCTTCAAAAATTTTTTCTACGAATTCCTTGAACG
ACTTACGTTCCTGAGCTACATTATGCTCGATTACTTTTTCAAGATTATGACTCATTCGAA
ATAATCTTCAATTTCATAATCATGGACGTAAATCATTATAGTTTTTAATACATCATCAAT
ACTTTTTCCTGGAGCTGGAATTACGTAGAAATATCCTGCTTTTGAGAGGTCTTTATAAGT
TCCAATCAAGAAATCATTATTCTCAAGATGTAGTTCTTCAACTAATTCATTAACAATTGA
ATGGTATAGGTTTGGTAGAAACTTATATAGCTTTTCTAGAATATCAATTTTGATTGTGTA
TTGAACCACAGACTGAGAATCAATAATCATAGACCTTCCCCTTATGTTTCTGTTTGCGAT
TAGATTCTTTAAACGCTTTCTTCTTATCCTTATGAACAGAAGCTTTATTAAAATTATGTT
TTGCGACTAAATTGTTCATAGTGCTGAATTACCTCTCTCAAACATTTGCATGTGAATGAA
AACTTTTAGCTACACCACATTCAAATATATGTTCTCTTAAATCACGTGTATCGGTATATC
CCATCTCAACGATAAAATGCCGTATTAGATTTTTATCTTTATCATTGAGAGAATTAAAAT
AATCGGATTTTGAATTAATTTCCCTGGCCAAATTGAATCACCTTCAGTTGACGTTTTAAT
TCTTTTATCATCTCTTCGTTCATCGCAATATAAAGATCGCGTAAAGCAGGTTTTAACATT
CCATTTACTGGAGAACTAAATGGACATACATAATCTTTTCCTACGAGCTTTTTAGTGAAT
TCAATATTACAGAACTGAAATGCCGGCTCATTGGTATAAATTCCCCAATTAGTTGACATC
ATTTTATTGGCATATTCCAGTGCCTGGATTTGATTCATAATTCCATCAATTTGAAACTGT
TTAATATTCATTAGTAAAGGTCCTCAGAATAAAGTTCTTTTTCACTACCACCACGTTCAA
TACGTACTTGTCCAGCGTAAGTTGCAATAATCATTGCTTCTTCACGTGTCCAATAATTAC
TATATTGGTCAATAAACCCTTGGTCATCATCACAAACTTGTTGAGTAACTAATTGAGGTT
TAACTACATCTAAAACTTCTGCCATATCTTTAGAATAATGACGAGCACCTGGAATAATAA
GAGTTCGTCCATCTTTTAATTTAAAACGATTAGCTGCACAGACAATTCGTCGTTGATACT
TTTGGTTTTCATCCCAGTACGCAGTCTGCCAACAGATTTCAGGAACTTCTTTCAGAATAT
CTTCTTCTGTGCATTTATAACCATGCGCTTTAAATTTTTCAACAAGACTTTCTGGAGTCT
CACGAGATAAAGGCACATTCAGCATTTTTAAACGATTAATAAATGGGTTCATTTAAACCA
TCCTTTAATACGCTGCCACAAAGTTTTCTGTTGAGCTCTGTTAACACCAATTGAACGAAT
AACTGGTTGAGATTCCTGGAATTCTTTATAATCGGCAAGGTAAATTTCGTAAGCTGCATC
CGTAAATGAACTTATCGCTGCCATAAAATTATTGCGAATACCTACTGGAGCATCTTTACT
TTCACGAATAATCATGTATTTACCAGTCTTAATCTTTACGATAGTTCCAAGATAAGCTCC
GTGGTACCAAATATCCCAACCCTCTTGAGTAGGTTCTGCGCAACGACGAAGTTCATTAAC
AATTTCTAACTTGTTCATTATTTATTCCTCACAGTTCAGATGCTACAGTGATTACTGCTT
CAATGTTTTCTACTGAGCGTTTAATGTCAAGATACACATTACCGTTTTTAGCGATTTTAC
ATGACATTCCGATGTCAGTAAATTTCTGAATATGATGTTCCATCATTTTGTATCCAAAAA
TTCGCATATTTCCGTTGTTATTAATTTCAAAATTGCGAATTCCGTGAGTGCGTTTTTCTA
AAATAGCAAGATAGTTACTGCGGTAAGTTTCAACCTTTTTAAGAACAAATCCATTTTCAT
CTAAAAGATTTAACATGAAGTCTTTATCTTCTTCCATATCGGAAGTAATCTCACGAGCTT
TACGAGTCGCTCGTTTTTTCAGTAGTTCAGGAGCATTTTCCTGTGCATATAAAGTTGCCG
CATTTGAAATAATATCCTGAGCTTCACCAGTAATGATTAATCCATCACCAGATTTCTCCA
CCAGGCCTTTTTTAATCAATACCCCAATATTACTATTAACTACTGCGTTACCTAAATCTG
GATGCACCTCACGAACTTCTGCAGCTGTAATGAAATCTTTCTTAGCAATGGTAATTAAAA
TCGCAGCAGTTTTTTCATTCAGAACATCGTTAGAAGCTTTGATGATGTAAGTTACTTTAG
ACATTTTCTAATCTCCGTAATTCTGTATCAGTAGTTGATAGTTGTATAGTACCACAGTAT
GCTTTGGTTGTAAACCGTTTTGTGAAAAATTTTTAAAATAAAAAAGGGAGAGCCTCGGCT
CTCCCTAAAATTACTGCATGACTGTGATAACTGTCATGATAACACGTTGAATTCCGAACG
CAAGAAGACCTCCTGCTACGGCTGGGACAACGCCTAAACCCGCCAGTAAAATGCTACCAG
ATACTAATGCAGCGCTTGTGATACCAATGAATGGACTCATTTGATTTCCTCTAAATCTTT
GGTGTATTCGGTAACTACATCAGTAGTTTTCCAATATTCGTTTTCTTCTTTTTTAGCTTT
AGCTTCTTCAGCAAGTTTCTTTGCTTCGTCGGAAGTCATATGAAAAATATTCATTCCAAC
TAGTTTATCAACATAAGAAGAATACATATCGATTTTCGAAAGTTCTTCGGTCAGTTCTTT
ACGAGTTTTACCCTGTACAACAATTTCACCTGAAATTACTTTCTTAATGAAATGTGCCTT
GGCAAAGGCTAAACGAAAAGCTGATTCAGTTTCTTTAATTTTGTTATCAATTCGTTTTTG
GACATAAGTTTTACGAACTTCAACAAAGTCTTTAATTAAATCGACTACGTTATCATAAAC
TTGCAGCTTTCCTTTCTCATTAATGACGGTAATATTCTGAGAACGACGCTCAATCAACCC
GAAGTCTTTCATAATTTTTGCATGGCGTTCTTCTTCATTATCGCTCAAAGAATATTCTTT
ACGGAATTTAACTTTGAATCCAAAACCATGCTCACCGCAAGCATCATCCCATGTAATGAA
GCCTTTATCTTCAAGTGGGTCTAAGATTTTACTCACATAAGTTTCACGATCATACTTATA
CGGAATCTCAGTGATATGCATTTGAGTTCGTGAAGTAAACTTATATGTTCCACGAATTTC
ATATTGACCGTCAATCTCAACGACTTCACCACGAAATTCTGGGAATTCTACCTTCGGTTT
AGTTACTTTCTTTCCTTGAAGAGCTTGTAGTACAGCTTTCTTGACAGAAGAAACACTATG
AGGAAGAATGTAAGTTGCATAACCAGTTGCAATACCGGAAACACCATTAAGAAGAACAGT
AGGAATAATAGGCAAATAGAAAGCAGGCGGAATGTGTTCTTTATCTTGATGTACCGGAGC
ATATTCAGTATCTTTATATACGTTATAGAAATTTTTACTTACACGAGCAAAAATATAACG
ACTTGCTGCTGCTTTTTGAACGGTACGAGAACCGAAATTTCCTTGACCATCTAACAGAGG
AAAGTTATTATTCCAAGTGTTAGCCATCAAAGCACCTGCGTCTTGCGCAGAGTTTTCACC
ATGATGATATCCAAGGTCTGCTACACCACCTGCAATAGAAGCAAGTTTGTGAAACTTATC
TTTATTTCCTCGCGCCAAATCAAGAGCTCGAGCAATAACAAATCGTTGAACTGGCTTAAA
TCCATCAATCATATTTGGGATAGCACGATTTTCAACCGTGTACATAGCATAAGCCAATGC
TTCATTATCAATGATACTTTTTAAATCGCGATTATTCAGTTGCATAAATTTACCATACTA
GTGAATGTAGTGCCATAATAACATCAGAAATGAAAAGCACGACTTGAATTAATCCGAACA
TTACTCCGTAATATAGTGCTACCAATAAAGTAGCAAGGGCTAATGAATAGCCCAAGATTT
TCTTAATCATTAGTAGATAACAACACAAATGTTAAATATGCACACATACCCTGGGCTAAA
GCTTGTGAAAACACACTGCTAGCATCGATACAGATAGTTAAAACACATGCTACTATCCAA
CAAATAAATGAAATAACTCCTAATAATTTTGCGATATTCATATTTTCCTCACTGGCGTCC
GAAGACGCCTTTGGTTTTAAGATTGTTACGATAGAACTGCATCACATGTTCATTATGGAA
ATTACTCATTAATATGCCTGCAAAACAAATTTAAAGTTATCAGCCAACATACGGTTCATT
TCTTCAAGTGTTTGATACTCAGAATGATGATTACGAGTAAATGCCAAAGCTAACTGACCT
TTACCAAATCCTGTCGTCAGAGGTTTCATCTTAGAAGCAGGCAGATAAAATACTGTGTAT
GGAACATTGTTATTTGCAATAGTACGTGCAAGCTGAGACCGACGTTGACGAATATGACTT
AGAACTGCACTGAATCCTTGCTTAGAACGCTGATTACCTACATAAAATCGTGCGGACACG
CATGGATTACTAAATGGACGACCATCTAATTTACTCACTAAAAAGTAAAATCCAGGTTTA
GATAAAATGTCTTTATGTGGAGTTCCCAAAAACCACTCACCGCCCTTGATTGTACCAATA
ACAGTAGCGCCTGCATTATTCAGATCAGTAACAGTCATATATTTCATATTAATTTCCTCT
AAATTATTTTCTACTCCAAGGCCGCATGAATACATACGGCCATTAAATTAATCGTCGCAG
TCGACGCTCAATTCCCAGAATTCTTCTACAGTATAAGTTTCAGTATCATTTTCAATACAG
AAACGTTCATTACTGTTATTTGCTAAAGTAGCATTAACTGTCGGTTTCTCGCTAGTGCTC
TTAAGAGGTGAAATACGAATTAACTGATCACCGTTATCTAAACAGAAAATTTCACCAACT
TTTACATCTTTAAAACTTTTCATAATTCACCTCAAGGAGTATAAAATCCAAACGCAGTTG
TTGACCATCCCATCCAATATGGGAAATTTGCACCAATGTAAAACATAAGAATATAAAACC
AAATGTTTAATAAATTCATCATTTTACACCATTCCAAATTGTTTCAACTACGGATTTTAA
ACCATTTTGATGAATATCCATTCCGACTACCGCCATCAAATAGATTCCAACTACAACTGA
ACCTAAGGCAAAAATCAGCATGAAAATGAATAAAGCCGGAAAAATATTATCGAAAAACCA
TTCAATAAATGTAAAAGCACTGCGTTTACGCTTCATATTTTCCTCACATAAATCCAAAGT
AAACGTTTAATACATCAATCATTAAAACGATTGGGAATATACTCAAAACTACTAGTATTA
TAACTACATTCCATATAGCTTTAACAATCTTTTTCATTTTGTGTTCCTCCGTAGTTGATA
GTTGTATAGTACCGCAGAGGAACAGTCTTGTAAACAACTTTTTTAAAAATATTCGTAATA
AATGTGAATACCAATCACCACTGCCGAAACCTGTGCAACCCACCACGCACAAGCAATAAG
TACAGAATTCAAAATTTTCATAATAACCTCATCACAAAAGTAAATGTTAAACAAATTACT
GGAATACTAATTAACCAAACAAAACACCACCACAATGAACTCATAGTTCAATCTCAGCGA
TTTTCATTTCATTACTATTAATAGCCGCTTTAAGACTATCTGAAAGAATTACACACCAGT
GGTCATTCATATGACCATTAACTAAGCGTGTAATTTCTTCGGGAGTTGAAAAATAAGGCG
TATCAGACTCCCAATGCGATAATCCCAAGCGAGTATAAATCATACCTTCATCATCGCTAG
AATATTCAACTGACACAAACTCATCAGTTATTTTATGTTTAGCGTAATAAAATTTAAATT
TCATTTTTACTCCTCCGTAGCTGATAGTTGTATAGTACCACAGTCCTTGTGGTATGTAAA
CCGTTTTGTGAAAATTTTTTTAAATGGAAAGATACCATCCGTTGTAGTTGCTTTTTCTTA
CAACCTTACGAAGGTCTTCTCTATCACCGATGAACTTCGGAGTGTACTGGATGACACCTG
GATGAATTTCTTTAGTGTTGAATATAATTATACAGTCAGCGACCTGATGATTCAGAATAG
GCCCTAGATTTATTCCAGAACCGTATGGATACTCTCCGCTGCATCCTGTTGTTACAGAAA
TCCAACGTGAGTCAGTTTGATGTGTCTTAACTTCTACACGAAGCCCGCAGTATCTTGGAT
GCGCCAATACATCCCATGCGTATGTATACGGGTCATTAACATCCTCTTGGCCTTTGTTAA
CATACCCGCTTAGCCAATCTGCTACAAAAAACTCTGCGTACACCGCGATACGACATCTTT
CGATAACTTCTGCTTTATCCTGGTTCGGGTTTTGTTTTAAAGAGTATCTTGCTGTATCAG
CAATTTTGACCTTCATTTCACTAGTCAAGTCACTGTTCGATAGGGTAAATGTCGGAATCT
GAAATAGTCTCTGTAAACCCGGATTCGTTTTCTGCATTTAGACTTTCCTTTTTACCGCTG
AGATAAGCGTTATATACTTTAAGAGTGCCGTAATAAATTCGGTCATTTTCATCTAAAGAC
TCACGATCAAGTTCATCGAGTTCCTTTTTATCCATGACTATAATATCATTGCAGTAAAGA
AACCCAAGTTTTTCGTGACCAAACTCAAATTTATTGCAGTACACAATATTAGCGTGATGG
TTTCCGTGCAGTTTAATCTCTTTTATGTCGGAATCACCGATATTCATACAATAAATCATA
ATTTTTCCTTAAAACAAAAGGGCCGAAGCCCCTTATTTTATTTGAATTGTGCAATTCTTT
TCTCTAGACATTCAGCATAAGATTTCATTGAGATGAACTGTGAAAGTAGCAGTTCTTGCT
CAACTGCACTAACTGTTAGAAACTTTGCGCTTTCTAAAAATTTACTCAGTGCATTAATTT
TGAGCATTAATTGATCGTATTCTTCTTTTACTCGTGCTTGATAACCTAACATAAATTTCC
TTTAGTCAAGGGCCGAAGCCCTTATTTAAATTGCTCAGTAACGTCTTCAACTACTTCGTA
TTGGCAAGTACGCATTTTAGCATCGTTGTAATCAATCGGAATTGATACTACATCACGTGG
ATGTACTTTAACTTTTACAACTCGGCTAGTTGAACTACCAAAGTGACGAATATAAGATTT
AGAACACACATGCAGACCACGAGAACAAGTTTGTGTATCATCGTCATTCACACGAGTACG
TGGCATTTTAACTACTTTACCAGGACTGTTATCAAAGGTGTTTGAGTGACAGTCAAAGTA
ATTGCTGCGAACTACTTTCCAAGCATAGAAGTAACCATCTTCTGTAATTTCAATATCGTT
TGCTACCAAGAAATCAAAGAGTCGAGATACCGCTTTTTGGCTTGGGTTTTCCAGCAGATT
TTCCAAGAACGGGAAATAAAATTCAAAGTTTTCGCCTTTTTCCATCGAGTCAAGAATACG
ATCAACCAAACCAGACCGCAATTCGATATTTTGATAGAACAAGCTTCCACCTTCAATTCG
AACATCGCCGGAAATATATTTTTCAACAGCACGGCGAACATTAATTTTTTGTGCAGCTTC
TTCCAGCCTATCCGCTACAAGCAGATTAAGAATTTCCTGGAAGTTTGAATGAGTATTAGG
AGTTGCGTTATAAGTTACACCGTCAACAGTAATTGAAATGAATTTTTTAGATGCATTCCA
AATAATGTCAGATTTAGCAACTGGAGCAATAACTGCATCGCTATTAACTTTAACTGTAAT
ATCACCGCTAATAGTAACTTTAGAGCGTTTAGCTTCTTCAGCATTTTTCAAAACACGACG
GATTGTGTCAACCGATACATCCTTCCAATAAGCTAATTCCTGTTGGGTATAATTACCACT
TGAATACAGTTTAACAATTTCAGCTTGTTCGTTTTTGGTCAGGCATTTAATATTGTACAT
AATTTTCCTTATTAGGCCGCAAGGGCCTTCATAGTTTTAGCGATTTGGGAAACTTCATCA
TCACTTAAAGAGTTGCGATAACCGATGAAGTCGGAAACAATACGGAATTTCTTGGTAAAC
TCAGCAACCATTTTATCACTATTTTTTGAAGCATTACGTGATAATTCATCAAAAAGATTA
GTTACCGTCCAGATGTCATGACCGATGGTATCTTTTCCACCATTAAAATATACACCGCGC
AATGAACTAACTATATTAGCGAGTCGTGTATATTCTTCAGAAACTTCATCTGTACTGAAG
TACTTCATCATAAAATCTAGTTCAGGATGCTTGATAATTTTATCAATATATTGTTGAGCT
GAACTTGAATAACCTACATACTTATCATAATCTACATCATCAAAAGCATCTACATATAAA
TCACGCAAAGCTTCAAAAATACATTGGCACTGACCGAGCTCTTTTACCTTTTTCTGCAAA
AGCGGACGAATAACATAAAATTCATTAATGCCAATAAGATTAGCCATACGAATCAAAATA
TTCATAGATGGATGACAAAGAGATGTAGTACCATCCATAGAGAAAATATCAGAACGATGC
ATATACGCTACATACCCAGTAATTTCATCTGCTTCTGATGTGAGCGTAAATAATTCCTCT
TTTTCCCAGCGCCCGTCTTTAATTTCAAACTTAAATGCAGTAGCAGCTTTAGGACGAGGA
GCTTTACTTTTAACTGCCTTCGGAATATAACTTTTAACTAAAGCTTCAATTTCTGACAAA
TAATGAATGTTAACTTCATCACTTTCAAACATTGCCATAATATCAGGGAGTAAATCAATC
TGCGATTCTACTTCCGGATTAATAAACAGAAGACGCTCGTTGTGATGAATATTTAAAGTG
TTATTAAATTCTTTATCATCTAACGCATGCGCTAATCCACGGACAATATTAACACGATTT
TTAATATTATCAATAACGATATTAATTTTTGTTGTATTAATACCGAACAGACGATAACTT
GATGCAATGGCTGAAGTTTCATGACTTTGCTTAATGCGCTTCAGTCGAGGGTCAAGATTT
ACTTCATACACAACTCCTGCATTACATAGTTTACTATCAGGTTCAAACATGCTTTGCATC
TTCTTATATGACAGATTTTTAGTCGTAAATTTGACTGAATTACTAATCATATAATCTCGA
GCAGAATATCCCATCTTCATCAATTCGCGGTATGTATGACGAGGAGATGTAGATTCTTTA
AATCGTTTTACATCTTCATTGAATGCCTTCTCACTAAGCTCTTTAACACGCTCAATAATA
TTTTTACGGGTACGATCATCTAGTGAAAGAGCTTCGCGTGATGGAGCAATATCAAGTGAA
CCCATTGGAAACTTAATGTAATTCACTTCATTGCGGATGCTTAACCAGTTACGGTCTCTA
ATAACACCATCAATAGGATAAACAATACCACCATAGATAGCATACAATCCACCACGATCT
GGCCAATATCTTTCTGGATTTACGCCGTAATAGTCATCAAAATCCGGAAAATAATCAATT
TCGCGGTCAAGACCATTAATGATAGCCAAATCTTTGAATGGTCGCATGATATAAGAAACT
TCATAAGCAAAGTTTCTAAAGTCTTTTTCTTCAACTGGAACTACAATTTCAATACCAGTT
TTATCGTCTGGACCCATTTCTTTTACGAATGTAGGTTTAATTTGCGGACCATCACCATCC
ATGTAAGCTACATAACCACGAATTTCGCCTTTATGATATGAAGTAATACTAAACGTATCA
GTATAGCTAAACGGAGATTTAGAACCTAAACCAAATCCGCCAATGAAATCGTTAGATTCA
GCCTTAGATGAACTAAAGTATGAATTATACAACCCAGGAGAATTATCATCACCTTGAATA
TCAAAATCGCTCATACCCGGACCAAAATCTCGGCAAACAAATCGCGGATCTAATCGTCCT
GGAACTTGAATGATAAATTTTTCAGGATTTCCATTGAGAGCATGGGCATCAATCATGTTG
GTAATCAATTCACGGACTACTGCGCGAATCTTGTTTGTATACAAATCAGATGACAGAATT
TTAAATACTTTAGGAGATGCTGTGATGCTAAATGCTTTTGATTTAGAACCATTACCAAGA
ATTGTTTCTTTTTCAGTGGTGATAATCATAATTTCCTCATTAATTCATATTACGCTTAAT
AACTTCAGCAACTTCTAATAGTTCATCTTTAGTTGCACTGTCGGATTGAATTTTATCTCT
AATATCTTTAAAGCGGTTTTTAAATTCTTCGGCTTCTCCCATATCGAAAAAGCGTTGAAT
GATTCTATATTCTCGATGAACTGCTTTATCAAAAAGTTCTAAATTTACTTTATATGATTT
CATTTCAATATCCTCATTTGCCCAATTAATTATACCACATCCTTGTGGTAAAGTAAACTA
CTGGCTCATCCATTCTTTACGAAGATCAGCATTATCTCCCATGAGCATTTCAAAAAGCTC
TTTCCAGTTCTCAGGAAGTTTAACAACATCATATACTGGGTTTTGAATCATCTCACGATA
TTCAGATTTTTCCAAAGAGCCAAGTCCCTTAATATAACGGATGCTATGTTTAGGTAGAGC
ATCTTTAGCACTCTCATATTCAGCGACTGTATAAAACCATTCTTGTTTTTTACCGACCTG
AGCGATGATTACAGGAGTTTTGACAAAGCGAATTCTTCCTTGCTCAAACAATTCTGGCCA
ATTACTAAAAAATCCGAGCAGAGAAGGATAAATGCTTCCTAGACCATCATGGTCAGCATC
AGTCATAATAGCGATATTATGATAATTCAAGTTTTCAGCTTTTTCACCGAGAACTAATCC
AGTGATTGCGCAAATATCAAATAGTTCTTTGTTTTTAAGCATATCTGCATAAGACATACC
CCAACTGTTGAGAACTTTACCACGTAATGGATAACCACCATGAAGTTCTTTATCACGAAC
ATCAATGAGATATCCAATAGCCGAATCACCTTCAGTCAAGAAAAGAGTAGTATCAGCATC
TTTACCACAAAGATTCGCTTTAATATGTTTATGAACCTTAGCTTTAGAGGCCTTTTTAGC
TGCTTTAGTTTCTGCTGCTTTTTCTGCCGCCAATTTACGAGCCAAAGCAGCTTCAATAAT
TGGCATTAAAATTGCTTCATTGTTTAAAATAGCGCGTGAAATCTTTTTAGCATCAAGCTG
AATATGACTACGAATTTCACCAAATGGAGAAGTCAAACGCTCTTTAGTCTGAGAATCAAA
TCGCATGTTTTTCATATCACGAACAAACATGACGATAGTCAAACATTCTTTAACACGTGC
TTTAGTTACATCAATTTTGAACTTGCGTTTGATTTGTGGAATAAGGTCTTCACAAATATC
ATCCATAACACAGTCAATATGATGGCCACCATTCTTAGTATGAATGTTATTGACGTATGT
TAATTGACGAAAACCATCCGGTGAACGACCAACCGCAATAGAACAATTTTCTTGTTCTTG
AACGATAGCATGCTCATCATATTGGCGTGCATATTTCTTAAAATTACCTTGAACCTTTTT
ACCATTAAATGTAAATTGAATATCAGGATAAACTACTGCAAGTGTCTGGAGACGATCCAA
TGTAATGTCAAGATAAACTTGAGATAGCTCATTAGTTTCAAATGACATAAAATCAGGAAT
GAAAGTAACACGAGTTCCTTTCCATTTTCCAGGAATAGTTTCCCATGATTTATTTTCCAT
GCCATTTGAACAACGAACTACAATATTATTTTGACCGTCGCCAGTTTCACCGACAAACAT
CACAGAAAAAATGTTAGTTAAACTAGAACCAACACCGTTCATACCGCCAGTGACGCGTTC
TTTATCATCACCGAAATTACCACCTGCTTTTGGAATAGTCCAGGCGGCAACAGGACCAGG
AATTTCTTCACCAGTAGGTGTTTTAACCATCGCTTGTGGAATACCACGACCGTTATCTTC
AACAGTTACTTGATTGTTTTTGATAGTAACATTAATTTTATTCGCGAATTTAAACTTAGT
ACGAATACCTTCATCTACTGAGTTATCGATAATTTCATCAATAAGCTTAACAAGACCAGG
TACATACTGAACACTTTCCCATTTACCAAACAGAAAGCGCTCATGCATTTCATTAGCAGA
AGAACCAATGTACATGCCGCTACGCTTTTTGATATGTTCAATATCGCTCAGAATTTTAAT
TTCATTCTTAATCATCACTTATCCTCGTTTGGTTTCGGGAATATTATACTCCGGTAATCA
TAAAGCTAAAGGCCCGAAGGCCTTTTATTTAAAACGGATAGTCGAATCCTTGAAGAATAG
ACCAGAACATACGGTTCCTTCTACTTTCTGCCCGGTAGGTCCAATAGCACGAAATCCAGT
ATGTTGGAAATCATTTTCAGAGCAACCGAACCAGTTATATCCAGTGATTTCAATATTAGT
AAAACCACTTGAAGACAAAACTTTGGTTGCATTATCAGCATCAGTACATCCTGCTAAAGA
CACTGCTAATACTAATGCTGCGATAGAACGATTAATATATTTCATAATTTCACTCAAATT
TAATGGCTTGAAGAAGACTAATAATTCTCAAGCGACTTCTTTCATCTTTAACCGTAAATG
AAAGAGGGTCACCAGATTTCATAGTGATAGTGCATTCAAAATCAAAATCTTCGGGAACTT
CTTCGAAGAAGTCAAATTCTTCATCATATATTAGAACATTACTCTGAAAACTGTGAAGAA
TTTTTCCATCATTTCCAGATGCTGTACTAATCATTGTAACGTTATTACCTTTCATATCTT
CAACGATAAATTCGCTTGTAGATATTACGGCATTAATAGAACCATTCCTATAAATAGCAG
AAAATAGATATTTCTTTTCTTCACCTTCGCGAATGCGATATTTCTTACCAATTTTAAACA
TAATTACCCTTTAAGTAAGTCGTAAAAACCACCATTCACATGCTTAGGAGCGGAAACTAA
CCGAATAGCAATCCGATGACAATCAGGACATACATCAGTATCCCTTTCAGAAATTTTCTT
GATTTTTTCGTATTCTTTTGCACAGTCTTTAGATTGACATTTATAATCATAAAGCGGCAT
AATTATTCCTTAAAGTGAGCTTTCAACATCTGATACAAAGACCACGCCTGTTCATTATTT
TCAATAGTAACTTTCATGACTGGGAATTCTGTGAAATCTTCTATTTGTTCTTGCTCTTCC
TGCTCTTGTTCTTCAGCAGCCTGATATGGATTTTCCACTTCATCAAAGAACTCAGCTTCG
TTAGTAGAAAGCCAGATAAAGTTTTCATCAAGGATATCACCATGTTTTGCATATCCAATT
GCACCAGTAAATGACATAATTTTAGTAGGACGACCAAGATGGTCAGCATCTAAAATTTTA
AAAGGATGCATACCTAAACGACGCGCATAGATTCCGTTATCAGTATGGTCTTTAATAAAA
TTTTCTTGAGCTTGTTTATTTTTAAATTGATACCATTTATTAACTTCAAATCTAATAGCC
ATTAATAAATTTCCTTCCAGTAAGTTGTACCGTCTTCAGTGATTTCACGGAATACACCAT
AAATCGGTTGTTTATCACCAACCTTTTCATACACATAAACCGAAGTCAAGTGAGTAAACT
TGATGGTGTGTTCCTTTTGAACTACTACCAAATCTGGATCGAATAATACATCTTCAAATT
CGTCATTAGTGCATTTCTGAACAATTTTACGTTTCATTACAATTTCCTCATTAATTAAAC
AGTGGAGCGATGCGTTTCAGAAGAGTAGCAGCACCTTTGGCGAATTTTACATCATGAAAA
ACAGTTTCTTCTTCCGTTACAACACGAGCTTTAATGTTTTCACAATCCCAATTTTTACGA
ACAATCTTCAAAAGCTGCAGTAAATCTTTGCATTCAGCAATATCGATAACACATCCACGA
GAATCTTCCATTATCGCACGATAACCGTAAGCCATACCATAATCATTGAAAGATTGCTCG
ATGATAATTAAGTTTTCCATTTTATTCTCCAAGTTGTTTTCTGTATCAGTAGTTGATATT
GATATAGTACCATAATCAACTACTGATGTATATAGTTTTATGAAAAAATTTTAAACTTTA
TGCATAGCGAGCTTTGCTATAGTGTTTAATCCAACTTTCAGGAATGACTTTATATGTTCC
TAAAAATACCGCGTTGTACAGCTTAACACCGTCTTCTACCCATTGATCGGTAATGTATGC
ACACATAGCGCGAGTACGGTTAACAACACCTTCCTCACCTTCAGTAAATTCAAACTCATA
AGGAGCAACGAACTTGATAGCTTGACCGAGTTTCCACTTAAAGTCTACACCTACATGCGA
AGTATCAATCGTTTCAATTCCTTTAGCAGGAACAGCTTTTAAAAACGCAGACTCAAGAAA
TTTCGCACGAACATAGCCAAACTGGGGTTTAGACTTTCCATCTTTAGGAATGATACGCAC
TTTTACTTCAGAATCTTCATCTTTAACGCCGTGTTTAAGCTGAATGCTTACAACTTCAAC
CAATTTTCCTGCTGCTTTAGAACGGGATTTATCAGATACACGAGCTAATTCACCAATATT
AATAATCATAGTTATCTCTCACTTGTTAAAAAGATTTTATACTCCACGGGACCATTATAC
TCTGGTCCCAAGAGTTTGTAAACTATTAATTCAAAATAGCTACCACTGCACTACGAGGTA
CTACGCTAAAATCACCTGCATGAACAACGTTCAGAAGTTCAACACCATCTTCAATCCATT
GGTCTGTTACCCAACCACAGACCGAAATAAATGGGGTCTTAATAGCTACCGCATGAGTCA
AAAGGTCAGTAGGATCAACTTCTTTGAAGAGGATAAGCTCTGCTTCAAAGGCCATAGTTT
TAATTACTTCATCATTATCTAAGCGATGTGCTCGAATCACGTAAGCTTTATTCTTATCAT
ACCAATTACTTTTTGAAAAACTTTTAACCACAAACTCGCCTTCACCAAAAACACTAGTCA
GTTCTTTATAACCTATATTTAGTTCAGTAGTTTTAATTTCAGGTTCAACCAGTTTGTAGG
TTTTGCCGATTTCAATAGCGGTAGTCATAGTAGTTTCCTTAATTTCCAGTGGTTTAACAG
GGCATACATAAGTGCTTACAACATCAAAATCAATCAGCTTAGCTACCGGATTTGGTGCGT
ATTCAGGATTATAATTAAATTTCATAATTATCTCATTTCAATAAAATCTACGAGTTCAGC
ATGCGATTTGCGGAACATTACTTGGTGCCCACCAATGATAACTTCATCTTCAGGAACTTC
GTATACAGCGAGATAAAATCCTTTAGAAGATAATTCTTCGCGCTCTTCTCGTGTGAACCA
TCTCATCATATCATATTCGCTAGCGAAAGCAAAATGATAAAGAGCTATAAACCATCCTGG
AATATAGTGATCTACGCCTACATATTCTTCTTTCTCAAAGTTAGTATTAATTGCTATATT
AGCATTTTTAACTAATAATTTGTCTTCGTGCGGAAGAGGAATTCTTTTATTATCATTACT
ATGATGCATAAAATTAGGTCTGTCATAACCTACGTGTAATAACCACTCTTCGCTCCATGA
ATCTATTATACTCCTGTATGGCGTTATTTGAACACAAAGATTTCGACGTATTGTTATAGC
ATCTTCATTATTAAGAATACTAAAAGATGATTCAACACGATAAATTTTCATTTTATTATC
CTCAGTAGCTATGGTGTTATAATACCATAACTAACCGAGGAAGTAAACAACTTTTATCGT
TTTGTTGGAAAAGATAGAGGATCGCATTCTTCCTCTGATGGAGCGTCTTCAAGACCCATA
GCATACCGCAAAGCATACTTCATCATCAGGATGTCTTTCGCACAGTCATGAATAGAATCA
TGTGCAACGAATCCATCTAAAGTTCCCTTTGGAAGAGGGCACGTGGTCATATCACGAACA
AGCAGAAGTGCTTCAATTCTAGTACGAATATCACGCTGATTCCAGAATTTACAAGGTTCT
AACTTAAACGTATCAAGTTCATTCTCAGAAACACCATTAAGACGTTGAACATCACGAATG
AGATCGACTAAAATTGGAAAATCAAACGACATTCCACGGCACCAGCCTTGAGATTTCCAA
GGATCGATATTATGTGCATTGATGTAATCATTAAATTTCGCAATACCGTCGATAGTGCTT
ACATCTTCATCGGACGGTGCAATATTTTTTCGAGCTTCAGGAGATTGATTCTTCCACCAT
TCGATAGTACTTTTAGTAAAAAGACGATGTCCTTTTTGACTTTTTAAGTCAAATTTAATT
TTAATCCCACGTGAAACTAGTTCATCGAATGTTTCAACAACTTCTGGGTTAGGGTCAAAA
GCAATTACAGCCAAATCAATAACAGCTGCTTTTTCACCGCTTCCCATTGTTTCAAAATCT
ATAATAAAATCAAACATTAAATTTTCCTTGCTAAATCGCGAATTTGACCTACAGTATAAT
CTTGGATATAAACTTTATTAATAGGCTCATCAATAAATTTTGCCATAGATTCAATATCTT
TTTGTATCTCTTCAAGACTGTATACTATCTTTAAAGCTTTTTCGCGAATAGTAATATTTT
CAGGACTAGAATTTTCCTCAATGACAGCTTTAACATTTGTCATAAGAGATTTAAACTGAT
GCAAACTTAATTCAATCATTAATAACCGCCTTATAAAGATGGCTAATTTCACCTAAAACA
TAATCACTGATTGTAACAGTTTTCACTTCGCCACAAAAGAATTCTAACGCAATTAAATCT
CGTTCAATTTCTTCTAATTGAAGCATTAACTTTCCAGATTCAATTTTTACATTTCTACGA
TTTTTACTATAAGCTAATTCATAAATTTCGCTAACTTTATCTTGAAGCAGATAAAACTGG
TCTTTAGTTATTTCCACGAATAGCTTCCTCAAATTTAATCATACATAAAATACATCATAA
CGACCACGGGTAACACCAACATAAAGAAGTTGTTGGGCCAATTCAGCATCTGCATAATGA
ATACAAGGCGTATAAATGAAAGCACGGTCTACAGACATACCCTGAGCTTTATGGAATGTT
GATGCAGGAAGTGCTTTCACTTTACTGAACTGTGATTTAGCATCCCAAAAATCACTCCAT
GGAGCTTTTCCGCCTTTGTTCCAATTTTTATAAGTTTCTGCCGTTTTACCTAAAAATAGG
TTAAACTTATATAGCTCTTCATCAGATGAAATTATTTTAATCTTTTCACGATAATATTCA
TCATCGCCGTAAGTTTCTACTGTTAAATCCCAATGACGAATTAAGTATTCTCCAGAAACA
CCACGAGCTTTAACAAAGGTTGATGTATACTCTGCTTCTATAATACGAACTAATTGTCCG
TTATTAAAAATAATTTCTGACACAGGCTTTCCATCAATTTTATATGTTTTAATTAATGGT
TCCTGCATTACAATAATTTCACCGACAATAAAATCTTTATCAGTTTCAAAAATCTTTTTG
CGAATAATGCTATTTAACTTGTCAACAGATTTATTCGTAAATGCCATTACGCGATTTTCA
AACAAATCATCTAATGATTTTACGATTGAAAAATAATTTACCATAAAATCGCGTAAAGCG
GTATCACCAGTAAATCCACGTACTCCATGCCCGTCAACAACTTTATCATAAATCCACTTA
CCGTTGCGAACATCAGTAGCTACATCAATAATAGGAGCATTACTGCGTTTAACTTCAGTA
AGTTCACACTGATAAAAATCTTTGTGTGTAAAGAATGGACTGATATAAGCAGTATTTTCT
CCTGGATCAACAGGTCTAATTTGCTTATTATCGCCTATTCCAATTATAGTACACCACGGC
GGGATAGTTGAAAGCAGAATTTTGAATAGCTTTCTATCATACATTGACACTTCGTCGCAG
ATTAATACCCTGCATTTAGCTAAATCCGGTACTTCCTTCTGTTCAAAAAGAACGTTTTCT
TCATATGTTACCGGGTTAATTTTAAGAATACTGTGAATAGTACTCGCTTCTTTCCCCGAC
AGTTTTGAAAGAATCTTTTTAGCTGCATGTGTAGGAGCTGCCAAAATGATACCAGTTTCA
CCCGTAGATATTAAAGCTTCAATGATGAATTTGGTAAGAGTAGTCTTACCGGTACCAGCA
GGTCCATTAATAGTTACATGATGTTTCTTTTCTTTAATAGCCTTCATAACGATGTTAAAG
GCATTTTTCTGGCCTTCGGTCAAATCATCAAATGTCATCGTAAATTCCCTGCAATTGGTA
TACTAACAATACGCCCAGTATCTAAAATTCGCTGATATAATCTTTGTGTGTCTACGTCAG
GCTTAACATGTTTAACTTCTATTTTATTAAACCAAAATTTACGTGGAGTCTCAACTAATC
TTGGAATTCCCTTACCTAAAGCTAGTTGATACTGCTCTTTAAGAGTGGTAAATACTTTAT
CAGCAATCTTCCATTCAAAAAATACAGCAGGACGATGTTCATCAAGCGGAACTGGCGCCG
TAAATCCGTCTTTGTCTCGGTAAACTATCGCGTATACATAAACCATATTATCCTCGGATA
AGTTTAAAAATTGAACAATTTAGCGGATATCCTCTTTTCAGTTTAAGTTTATCAATAAAA
GACAAGTTTTGATACCGCTCTACACCTTGAATAATTTTATCACACATATCATATTGCATT
TCTGCTTCTGACAACTTTTTCACAATTTTCCAATCCGAACCTTTAAGAAGAACGTTCAGT
TTAACAACTTCAGCGCCTTCTGCTATGCGAGAACCGTCAATACGAGCTTTAAGTGCTATA
ATTCTTAGCTTAATGTCAGAGGTCTGTTTTGATTTAGAAAGCTGAGAAATGTGTTCAATT
CGGTTTTCACGTTTTTTCTGTATAGCTTTAATTTGATTATAAGTCTTTTTGATTTTAGCC
CATTTCTTTTCATCTAAATTTAGTTTATGAACTTTTTTCGCAGATGAACGACCAATTCGC
AAAGCAAATAAATCACGCTTTTCAATTAACTCTTCTAAAGTATAATCAGAACGAAATGTA
TTATACTTTTTCTTTACTGCAATAACATTCCCTTTAATGTATCCAACGTTATTATCAAAA
CGTTCTAATGATAATTTCTCTCCTTCAATACGATTATCAAAAGGTTCTCCCGAGTAAGCG
CAAACTTTCTGATCTAAAATGTTCTTAATGTAATTGAAGTCTAAGTTAAAATCTTTAGAA
CGTCTTTTTGCAGATGCCTGAGTATGCTCTAGACGACGTTTAATTTTACGAATTTGGTTA
TTAGACAGCTTCATATTTTTCTCACATCTTACGGACGGTTAACTACTTATACTATAACAT
TTTTACCTTAACTTGTAAACAACTTTATGTAAAAATGCTTTAAAACTTTCATGGTATAAT
GAATCTAAGTCCTTCCATTATAGATTAAATCCTTCAAATTCAAGAGTATAGATAGTGTAT
GTTGAATACTTTTTGTACTCGTATCTATCTGCAATTCTAAATACACTTCCAGCTGGTATC
ATTACTTCTTGTTCATCTGAAACTAATTCCATATTACGATAACGATGGCTATCCGGAAAC
TTAAAGTTTGGATTGTATTCTTTACAGCGTAGAGCTTTTATAGCATACTCCTGGAAATTG
AATACCATGGGAGCTTTGAATTCAAAAATAACTTGTGTGTTATACTCTAAACCGGAAGCA
AAATGCAGAGCTATATTTTTATCATATGAAGCTGATACTACTTTATCAAATATAATAACA
TCAATTCCTTGATTTAATACTTGCTTAGTCTCGACTGGAACACCTCTCCAAAGAGGTTTA
TCGTTTGGAACCAAACGAGATTTGATTATTTCATTTAACCAAGTATGGTCATCTGGTTTG
TTGGTAATACAATGAATTAAAAGTTCAATTTCAGATAAATTAAACCCTTCAGAAAGTAAT
TCTTCACGAATAGAAGCACGCACCGATGCATCCATTGATTTTATTTTAAAATCTTTTAGT
TGCATTACTGAGTATTTCATTCAACTACCTCAATATCATAAACTTTAAATGTTCCAAATG
AATCGTGTAATTTTTCTTTTGAAATAGAAGTTATTCTATACTTTCCAATTGGAATCATCC
ATTCTTGTTCACGCACAATCATCATTAAGTTATCAGTACGCTCTGAATCTAGCCCATCAG
TATCTTCATACGTGTACTTAAACTCAGTATTAGGAGAAGAAAGTATAATATCGCTGATAT
GGTCAGAATAATTAAAAGCTTTATCAGTTTTTAAGCGAAGTATTATTTTAGTAAAATATT
CAGCATAAGAAAAAGAACATGCTGTGAATAAACTAGTAGTAAATGAATCTACCCTGTTCG
TTGAAAACACTTCTCCAACTTGTAAATCTTTAATAAGTTCTTTTGTCGATTTTGATATAC
CACGATATAACTGATAAGGCGATTTAGTTAAATGCTTTTTAACGATTTTATTCAACTGCT
GATGAAGAGCTTCATTCTTTTTGGCTTCCATACATTGCCAAAGAATTGACTGCTCAAAGT
CAGTAAATTTTTCACAGACTTTTTTATACATATCATATTGAAAATCAACGCTTTCAGCTT
TTATAGATAACTGTTCAACATCTGCAAGATTAATAATCATGATAGCCTCCGTATACTTCA
GAAGCTATCATATCATCGTTAGAAAGGAAAGTAAACAACTTTTTGAATTATTTTGCCCAG
GGAGCCCAGGGCGGAGGGTCAAGATGGTATGAAGCTAGTTCTTCTAGAAGAGCATCTGGG
GCTTCAATTCCATAATTCTGTAATACTATACGGTACTCTTTCTTATAATCACTAGAATCA
TTCTGGATATTCGTAGAATGATTATCTTCTAACATCTCAAATAAATCCATATTAATTCCT
AGCGATAAAAACCAAAATGACGATTAGCTTCAGCAATCTTTCTTTCTTCTTCGGACATTC
TCCAGCGTAATCCAACGTCAAAATGAGCCCAGACCATCTTAACAAATGCATTTATATCTT
TAATATCTTCAATAATGAATGTTTTACTTTTCGAAGGTTGACTCGCTAATTTAACTTTAT
CATCTTCAAACAGGTCAAAAAGACCATATTCATGAGCTCTTTTATAGCCTTTAATAGTTA
AAGCTTCGGAAGAAGAAAACGGTGAATCAGTATTTTGCAAAATATCTTCAGTATGCTTTA
TAATTAGAATAATATTTTCTGGATATTTTCTTTCTTTTATATCTTTAATTAAAAAATCCG
GATTTTCTGCTAAAGGAATAATTAATGAGCAGTTTTTATCAAGCGTATTTACTGATTTAC
CTTCTTTAGACATAAATTCTATTGAATAATGTTTTGCTACTTCAATCATGTGATTTCCTT
TTGCCTACTAATGGACCGTCGGGAATTTTGTTTTCCTGGATATATTTCTCATTTTCTTCC
ATCATTTTACTACCAATTTTAAGAAGCAAATCCATTGCTTCATTTGCTTTTGCTTTAGCC
TCTTCCAGCGTCATATCTTTGCTCATGATTTATCCCCATAGATGTCTCTCATCAATTTAA
GCGCTGAGCGTTCTAATTTCTTTTCTTTCTCAGCACTAATCATTGATTTCATCCATTCTT
CCGATTCGTTCTGCATTTCTTTATTTGCTTGTTCAACCCAACCATCATCAATGTACATTG
AGTTTGGTCTATTGAACCATTCAAGAATCTTCTTGAGAGCTTTCATTTATTTTACCTAAA
ACAATAGTAGGAGCATTGTCAAATTTATGAATTTTTAGCAAATTTGGATTTAAATTATTC
CATAAAGAGGTAATAAAATATGATAGCGCACTTTCGTCCGTAATTATAAATTTATTTCCT
TTATCTATTTTCCAATCATATATTGAATCATATGAATAGAAGGACAATGGTTTATTATTA
TAATATGCTTCACTAACATCAATATAGTTAGATTTAGTAAATGCTTGAATTGCCATAAAT
GGAGAATTTTGTACAGTTTCAATAATTCCGATCTTTTTAAGTTTTATTTCAACATCTTCT
GGAATTGGCATTGAAATAAAATCTTCTACTAGATACATGTTATTGTCATATCTTTTATCA
ATCATTACTGCTACGGTAATTGGAACATCCTTGACAAAGGCCATACTAATACTATTGATA
GACATTTCAAACAAAATTGCTTCCATAATTTTCCTCAATCACAAGATGTAGATGAACAAC
TAGAATCACAAGAACTTCCACATGAATCCCCTGCCCATACATGAACAGGAACATTAGTAT
CATATGAATCAGAACTAGACTGTGTATTCTGTGTGTTAGATGATGTAGTAGGTGTTGACC
AGCGCCAAGGATTTTTATAATATTCTTGGGCTTCTTCATAAGTCATAGTAACTGCTTCTA
CTGTTCCATCTCCCATATAAACATACTCAACTACAGTTAAAGGAAGGTAGTCATTTGAAA
TAGGAACTACACCTTCCCCAGGAGTTGTAGAGAAAAAATCCGTAAAGAAACTTTTAAACC
AATTAAAGATAAACATTACAAAAAGCCTCTTTTGAATTCAACTTGCTTTTCACCATAATC
ATATCGAATCTCTGCATTAAATTCAACAGAACCATCTGCGTACATCATAAATGAATGCAC
AACAACTTCTGTAGACCATGGTTGCAATTCATATTTCTTCATTACATGCCGTGAAATGAT
AATATCTAAATCTTCATTCGGTTTAATCCAACGATTTAACATAGTGCTCTCCTCTATAAG
ATAATTCTATTATACCATACTCATTTTGGAAAGTAAACCATTTAAATGAAAAAAAGGACT
CCCGAAGGAGTCCTTGAATTTATTCACCAGTTACTTTCCACAAATCTTCATTTGCAGCAA
TCCATTCAGTACGTTGATTTTCTTCATATACTGTAGAATATGCTGCTTTTTCTGAAGGGA
ATGTCTGGTAATGAGCACCAGAAATTTTGTGAACATCAGAATAAAGAGGGAAAGCTACGG
AAATTTCCTTTCCTTCAATTTTCTTATTTCCATCTAATTTCTGCTCAAATGTTTTGATAT
TAACATAACCGCGAGTACTAGCCATGTAATTCTCCTTTTATTTAAATTACATGATTATTT
ATAATAAATTCTCGCGGAACGCTAAAATACCCAATTTTAGTTAATATAACAACATCGCCG
TTAATAATTACCGGAGAATAATCGGCTGGAGCATTAAATTTCCAATGGGCTCTAATATTA
TATCCATCAGAACCAAATGCTCGACTAGCCGGTCTCCCGGCTTTTTGCCATGCATAAAAA
TCAAATTTCATTTTGAATGCTCTATAATTAATTTTAAGGCTTCTTCGCGCACCAACTTGC
CTAATGGTATATGCCCAGTTTTAATTGTGTAAGCTTTGTGGAGCTGATTAGATAAGAGCT
CACATACGAGCTCCTTGTCATAATCTTTAAATAGCTGGTCAATACGTTTCTTGCGGGCTT
TCTTAAGCTGGCGTTTATTTAAACGAGCCATCAATAACCTCGGTCTTGACGAGCGAAATT
TTCAGCATTTTTCAGATAATAAAGTTTAAATATTTCTTCAGCTGACATTCCAAGAGCAAT
GAATTTATTCATGAAAAAGTGAAATTGGTCAATTAGTTCAAATTTAATTTCAAGTTGATC
TTCAGGAGATAAATCTTGAATTTTCTTGGATTGCATTTCAGAATAACGTTTCTTCCAAGG
TTTCCATACAGCAGAAGCTTCTTTTTCACCATTACTCATACCACCAAGAGAAGTATATAG
CTCGCGAGTTTCATCTGCGATATAATCATCTTGGTTTCGCAACCAAGCAAGAACTTCTCC
TGCAGTTTCAAGTGAATCAGGATGACGATTTGTTTCTGGCTTATCATTAGCCAAACGAAT
CTGTAAATGCCGCTGCATATCAAGCATAACCTGCAATGGGTCAATATTGTGCATAATATT
CTCAGCATATGCACGATTTGCTTTATCAACGCCTTCGATCAAATGAGCGCATTCATTAAA
GTGAGCCATTATTTTTCCTTTCAATTCATTAATAAGTTAAATAATTATATCATTTGAATA
TAAGCAATTAATTAAAAATATATACTTCATCAGTTCCATTCTTTTCTTTGGAATGATATA
TGTTAAAGACATATTTTTTATTAAGATGCTTAACATTGTATTTTTTAGACCATTCTTTAA
GAAGAGTGTTTTCCTTTCCGTGATGCTCTAAAACATTCGACAGTCCAAATTTTATTCCTC
TGTCATTTAAAGAATCTAAAAGATTTAAAAGGTCTTTTTCTTCTTCTTCTGACCAAAATT
TATTATAATCAGCAACTGTTATAAGATATGGAGGGTCCACATATACAAAATCGCCGTCTA
GAATTTTGACATCTTTAAAATGCAATGAACTAAAGATTATTTTATCACAATTTTGTTTAA
AGTGATTAAAGCGTTTTTCACTATTTTTGTTTATAGTTCTTTTTCCAAACGGAGTAGTAA
AATTTCCTTTATCGTTTATACGAATCATATTACTAAACCCGTGAAAATGAAGAACATAAA
GTAAAAGAGGATCTCTAGTTTTATTATAATCTTCACGTAATTTCAAAAACTCTTCTTTTG
ATGTTTTTGATAGTTTGTATTGCTTTATTACTTTTAATACGTCATCCCATGATATATCAA
TAAGACATTTATACATTTCAATAATTGGTGTTTGAATATCATTGGCCAATACAGGACCAT
TGACGTTCAAAGACACTGATAAACCTCCACAAAATAAATCCACGAATCTGTCATATTTTG
GAAAGTGGGGCTTAAGTTCAGGTAATAATGACTGTTTATTACCTGTATACGCGATAGCTC
CTAGCATTATATTCTCTCATTTATTGCAGCAAAAAATGAATTATACAATTCATCATCATA
TGTATTTGATAGTAATACTAACACGTTTTGCGATTTAGCATATTCATTAAACCTCGCCTG
AATAGTTGGATTATCAGTAAATGATTCTCTTTTTGTCCATTTATTACGCGGATAATATCC
GCATGGCAAAAAATCTAATATCATCAATGATACAGGGTTTTCAGTTTTTGTGTATTCTTT
TAATTTCTCTTTATCTTCTAAATATATTCCAACAGAATCAATAGCTGTACCTCCACCAAC
TTGGTGTTTAATTTCAATTATATGCTTTTTACCGTTGCGAGAAATAACAATATCAAATCG
TCTAGTACGCTTCTTAGTTATAAAATTCGAGCAAAACAAATTATTTGATTTTTCAGTATC
ATCGGTTAAAACCATTTTTTCTTGATAACTAACTGAACAATCAGTGAAATATTCTTTTAC
TTTATTAAGTATGAAATTTTCAACTACGGCAGATATTTTATATGAATCTCCGTTTAAAGC
CATTTCATGTACAATTTTATTAATCATTATATCATCCTCAGCAATTTATTTTATAATACC
ATAACAAGCTGAGAAAGTAAACAAATTTGTGAAATTTGCGATAAATATTAATTTAAAGGA
GGACATATGGTACAAAAATTAATGGCACTTGTTAATGCCATAAAAGGTAATAAAAAACGT
ATAGCTTTTACTATTTCTGCTATGGTAGGAATTTTACTCTGGAACTTTATTTTATCACCA
ATTGCAATTGCGCATGGTGTTAATATTCCAGTAGTTACTCTTGATACATTCGTAGATCTA
GCATTTGCTTTAGTTGGGTTAATTTAAATCTTGGCATATTTAGATAGCCGCATTTTAGCC
ATCAACCCCTGGGCAATATTATTTTTCATATATTCCATAATTTGTTCAGGGGTTGCACCT
TCCTTTCTAATCATATCATTGACATCTTTTGATTTCCAGGGAGATTTATCCCAAAACATA
ACCCTTTCTCCTGCATCAACTAATTTAGTCATTCGTTTGATAGTGTCAGGGTGACGAGGT
TCATTATCTAAAACCCACACTCGTCTATCTTTAAATGGAACAACTTCTAGGTCTAATTGA
CCACCCGTAATAGCTATACCATTTTCAATAAAAAGTGAATCTATAGGTCCTTCTAGAACA
TATACATCACCATCTTTAACTCGTTCGACTCCATATATTTTTGTTGCCTCAGGATAAGCT
TTGATGGTGATATATTTTTGGGGAGCATCTTTCTTTAATGCACGTCCTTGAAAAGACTCA
GCTTTTCCATTAGCATTATAAATTGGAATAACAAGACGAGGCTCAGGTATTTCCTTTTTG
TATGTTCCCGGTGCTATGCTATTAACTAATTTAGGCCATTCAGTTGTAAACCAAAGATAT
TTCCATTTATCCTTTGGAATACAACGAGCTTTTACATATTTTATAATTGCATGGTCTTCC
GCCAGTTTATCTAATCTAACGCATGACGGAAGAGATTTAATTATTTTCTTCTCAGGTTGT
TTAGAAAGTTCTTTAGGTTTTTCTACTGGACGACTTTTACCTTTTTCTTTTCTTATTTCA
AAGATATACTCACGATATAAATCAGGTTCAAACTCCTTTAAATATATTCCGATTGGTGCA
TGATAGTTACAGTTATAACAATGAATATTTCCTTCATTATTATCACCATAATACCATCCA
CGAGCTTTATTCTGGTCGGTTTTTGAATCTCCACAAACAGGGCATCTAAACCGTAATTTA
AAAGTTGAACTATTATTTACTTGTGTGAATTTAGGTAAATGAGCTAATGCACGGTATGCA
AACTCATTATCAATCCAAGGTATTGATGACATTTTTACTCTTCTTTTTCTTTAGATTCCT
CTTTTTTCTTTTTAGGAATCTGTTCAGGACCTTTATTTACTACAGCACCTGATGTTGTTC
CAGTAGAGATATTTTCAGGATTACCACCTGAATCTCCAGCTACCATATCTTCTTTGATAA
ATTCTTTATATGTTTTCATATTAACCTCTATTCATAAAAGCATTAAAAATTTGGTCATCA
ATAGATGGAATAGCAACCTTTTCGGCATCATTTAGCAAATCATTAATTTCATCAGATAGC
ATAACACATGTGCCTTTATTTCCTGTAATTGTTACTGCACAATTACGAACGTTTTCTTTA
TCAAATTTTAGGTTAATATTTTCGTAATGAAAATCCTTTAATGCTGAATATATTTTTAAA
ATAAAATTGTCTTTTACTACAATAAACCCAACCTCACTATTAAAACGTGGTTCATGGAAA
CTGCAATAAACCTTTTCATCTATGAAATCGTATGAAATTTTTTCCATTTTTAAATATGAA
AATTCAGAACTATGCATACTAACCTCTAATTACTCACCTACCACTTCAGCGATGATATTT
TTATTATTAAAGTTTTTATCGCAATATAGAACATAATTATACTGCATTACACCACCAGAC
TTAAGCTGTTTTTGCACTTCGGCTTTCATTTCAGGACGATCGCGCTCAACGATATTCATA
ATATCTGCTTCAATTTGGGTTTCAACTTCAGTCTGATCCGCAGTCATAGACCATTCGCAC
AAATCTTTATCATAGCCTGCCATAGCAGGCTGAGCAGCGCAAGAAGCTAAAGCAAAAATT
GTAGCAAAGATAAATTTTTTCATGATAATCTCCTCAGTAGTTTATGTTTATATAGTATCT
CAATTTCCAATAAAAGTAAACAGTTATTTTAAAACTTCTGCATAATCACATGTTACAAAC
TGGTTCTCTAACTTGACAATTTTACGAAAGTATCTTTTGCATTGGCGAATCTGCCGCTTC
GTAGGGCGAACAGCAAACTTAATAAATTCCACTCGACCAAATGGAGGACTTTCTTCTGCT
GGAATATCTAACACCAATTCCCACGTATCTGCAATAAGTGCTTTGAATTGCGTATTTTTC
CTGACGTTATACGGAGTAGGTTTAAATAAAACAATATGCATATTATCCTCGGCAATCCAC
TTCGCACACTTTCTTGTCATCAATGAAAGCTTTAACTAATGCTTTATTAACTTCAGCATA
TTGAGTAGTAGCCCATTGAACGTCATCTTTCATCATTGTGATTTCTTTAATAAACATGCT
TTCATTCTTAAACCACCCCATAAAAACTACCTTTACCAATTCCATAACAATCTCCTCATT
TAACCAACAAGACTACTATACCATAGTCTTGTCAGCTTGTAAACTAAAATTTTAATTCAT
TTGCCAAAGCATCTAACTGAGCTCGAGCTGATTCGTTTCTTTGATAGCGATTCTGTTCAG
CCTGAATCTGTTGTGAACCTGCTACTTCACTCACTTCGGTTGGAGTAGAATCTTGTTCAA
TTTCTACCCATTTTTGATTTCCTTTTTGAACACCCATCAAAAACTTATTCCACTTATTCT
TATCACCATATCGTGATTTGATTTGCTTAATAAGTTGTTGTTCAGCAGCTGCTAGCTCCT
CAGTTTCAATGACCGCAAGCATAAAATCAGCTGTTGCTGGAAGACCAGCAGATTCTGCAA
TATCGCTCATGTTAACATCGGAAGAGTCCCAAGCTTGTTTACCGACCTGTGCTGCAGTCC
AAAGAACAGTTTCAGTTTCAACAGCCAGAGCACGCAATTCTTCTGCGATGGCTTTAACAG
TTGTGTAACTATTTTCCGAATAAACTCTGATGCGGCAAGATTTACAAATACCCAGATAGT
CAACAATAATGATTGTTGGAACAAAATTCTTCTTGAGCTTCAATTCGTTTAAAAGAGATC
GAAATGTATTAGCGTCTGCTCCACCAGTAGGATATTGCTTAACGATTAAACGACCGAGAG
TAGATTTCTCACGCCATTTTTCCATTTTTCCTTTATACTCAGCGTAAGAAATATGCCCAT
CATCAATGTCATCAAGAGAAACATCAAGCATATTAGCGTCAATACGTTTAGCACAGACTT
CTTCTGCCATTTCCATGGAAATGTAAAGAACATTATGACCGAGCTGTAAATAATCTGCTG
CCAATGAACACAATCCTAATGACTTACCAACGTTAACACCAGCCATTAAAACGTTCAGTG
TTCCAGTCTCAGCTCCGCCTTTAGTAATTTTGTTTAGAATTCTGAGTTTAAATGGAACCT
TACGAGCTTTATTCATATAAGATAGCCAACGCGCTTCATAGTCATCCATCCAATCATGAC
CAACGTAGCTATCAAATGAAATTGATAATGCTTGGCGCATGATGTCAGGAATAGCACCTA
CATCTGGCATTTTCTTATTTCGTTTTTCCGGAGGAAGCTCAGCATTAGTTTGAATTTCGA
TTATTTTAGATGTAGCATTAAACATCGCCCTTTGCTGAACATATTTTTCTGTTTCTTTTA
CTAACCAGCTGTGGTCTTCTGGAGAATCAGCCAGTTTTGAAATAAGTGTTTTTACACCAG
AATATTCTGTTTCAGTAAATGAACTATTTTCTAATGCAACATTTAACGCATTAATAGATG
GAACGCTATGATACTCATTAACATGAGATTTAATTAATTTGAATGTATTTTTAGCTGGAC
CACTTTCAAAATATTCTGAATCCATATATGGCCAAACTTTTGAAAAATAAGCTTGATCAA
ATATGAGATGAGAAAGAATAATTTCTACCACACTTACTCCTTAAAAGAATTTAAATTTTT
TCTTTGACCTTTTATTAAATGCATCTTGCAGTTGCATTGTAATACATTTTTCTACATGAG
GAGCTAACTCAGCTTTTCTTTCTTGGTCAAGAACAGCAAAGTCCATTACGACTTTTCCAT
CAACCCAATCTAATTTTGTCACATACACAATGTGTGTAGAACCATCTTCTAGTTTAATGA
CAATCTCCTGGATAACATTTTCCATAGCGGATTTAATTATCTTAAGAGACTCATTAAAAA
GACGTTCTTTTCTTTCTTTTTCCCCCTCCGAAGAGGGGGATTCATCGATAATTTCTAGAT
CTAAATCTAAATCATCTTTATTCATTAAATTCTTCCATATCACTTAACTGTTCGAGGTCA
GTTTCTAAATCAGCTGCTGATTTACTTTTACTTTCTGGAGATTTAAATTTTTCAACCTTT
GAGTTAATCAATTCATCAACTTCAGCTTCAACAATTTCATTGCTATCAATAGCACCTAAC
TGATAAGCACGTTTAATAGCATCTCGGAATGGTTGATGCTTAAATAAAGGACCCCAGAAT
GTAGTGCAGTTGGTATCTTTTGCACGCCAAGATTTTTCTTCGCGAATCATCTCGCCGGTT
TCTTCGTCAAGAAATTCACGAGCATACCAGCCATTTTTAGGTTTTACCACGAATCCTAAT
TCTAGAGCCATATCTAACAATCCAGAATAAGGATCGATACCACCGTCAAATTTAACATCA
ATAAAGAATTTACTTTTTTCTTTAACGGTACGAGATTTTTCTACATTTAGAACAAATTGA
TACCCCTGAAGATCAGAACCATCTTTAATCTGACGTTTACCGATAATGAATACGGTATCA
GCCGAATACATTACGCCTGTACCACCTGTCATTACGGTTTTACTAAACATTTCAATTGTT
TCAATTGTATGGTTAACCGCAACGCACGGAATATTTTTAATGCTAAAATAAGGAGTAACA
ATACGGAATAATGACTTCAGTGATTTAGCACGAGTCATATCTGCCACAGATTTTTCATTC
AAGGCATCTTCCGTTTCTTTCTTAGAAGCCATATTACCGATTGAGTCGATGAATACAATA
ACCTTTTCACCACGCTCAATAGCTTCAAGCTGATTCACCATATCAATTTTCAGCTGTTCA
ACTGACTGAATTGGTGTATGAATTACTCGTTCCGGGTCAACTCCCATGGATCGCAAATAA
GCTGGAGTAATACCAAATTCGCTATCATAGAATAGACAAACCGCGTCAGGATATTTGTTC
AAATATGCCGCAACCATAGTCAAAGACATATTTGATTTAAAGTGTTTAGAAGGCCCTGCG
AAAATAGTTAAACCAGACTGCATACCGCCATCAATCGCGCCAGAAATAGCAATATTAAGC
ATTGGGATTTTTGTACGGATTACATCCTTTTCATTAAAGAATTTAGATGTAGTCAGTTCA
GCAGTCATTTTAGAAGTTGAAGCTTTAATCAAACGGGATTTTAAATCTGCAATAGACATT
CATTTTTTCCATAGGCATCATTATATTTTCCTCACTGGTTAAAGATAGAGTAATTATAAC
ACAATAAATTTAGGCATTAATCAACTGCTATTGGATGAATAGCATTAAACTTATGAAATG
CTTCTGATTTTTCTTTACGCGAAACACACATGCGAAGAACCTTTAGTGGTTCATCTTCTT
CACCCAACGATTTTCGTTTTTCAATATTAGAAGTTTTCCAACGAGCTTGCTCTGGAAACT
CTCTATTGATTTGCTCAAGCGCTCTATTATGTTTAGAATTACTACGAATTGAACTACATC
CGCCAGGAGCTTGTGCTTTTCCAGATACAACTAGATATTTGAACAAGGCCAAATGCGGAT
AACCTTGATTAATTAAATTGAGAAATGCATACATATCTTCGCATAAATCAATTTTTCCAT
ACCCAATTTGTTCTGTAGTAAGTTTTCCGAGGTCATACCATGTATTCGTGAATCCATATG
AATTTTCACGATAATTACCCCAAGATGAAGTAATTTTAAAAATAGGTAGACGAGCATGGC
CATGATAATACCCACAATCCATGGCATCCTCAACATATTGAATCAATTCATAGAACTGTT
CACGAGTCAATTGATTGACTTTATCCACACAACGACGATCATCTCTTTTTCGCATTGAAC
TCATACGAATAGTAGTATCATCGTCAATCATCCAGATTCGTTGACCTGCATACATATCAG
TAATTGCTTTACGAGTACCGGCAATTCCGTTTACATCATCAGGAATAGTTATAATTTTAG
CTCTAGACCCATAGGCATCATAATAAGCTTTTTCTTCGTGTTCACGCACTACAATATGCG
GTTCATAATCAGTCGGAAACATATCAAGGGCAGAAACTGCCCCGACACGCTGATAACTTG
GAATTACGAATTGAATCATTTCCACTCACCGTTATAATCTTTTTTCACAATATGAGTTTC
GCCAGTATTCCACCAATGATCAACCAAGTAAAAATGACGAGAATATACATGAAGACTTCC
AACATTCCATATAATAGAACCTGCTTTATACTGACGAGTTGAATCGCCTGCATTCAAATC
AGATACTAATTTATCTAATACGTATTTTTGCCATGCATAATCATTACGGAATCCGAAGAC
CACGTCATTTGAGCGCATGCTTACTACTGCATTGACTTTCTTATCACGAATCAGGTACTG
TACTGTATTAGTACACATGAAATCTGACATACCATCTTTATTGTAGTCAAATTGCATGGA
TGGACGAGTATAAATCATGATACCACGTCGAGAATCAGGATTTTGACCAAGTTCAGCTAA
ACACATATCATACTGGGCATAGTTATCTTCTGACCAAATAGCCCAACCATAATTCGAGTT
AATTTCGCCTTTAGAAGATGCTACCTGCTGCCAAATCTTTGGTGTTTCACCTGGAATATC
TTTAACAAACAAGCTCTTAGATTTATACCATTCAAGTTCACGCTGAATGTATTCATCATT
AAGAGCGCCAAAAATAAACGGTTCATCTGCTACAAATGATGCGCCAATAATTTCAATAGT
TTTAACACCAGTTTTATCAACTACGAAATCTTTTTCTTTTAATGCAAGCCCCAAATGAAG
ACGGATTTCTTCAACTGTCATAGAGTCACTAATCATTTAAACCTCAATTGATACATTCAT
ATTTAACTTGTAACAGTAATAAACCCCAACCTAAAATAATAGTTGGAATCATAAGAGGAA
CCGTTACACTATAGTATATACTTATTATAATCATCAAGATTAAAAGCAACACTGCTATAA
TTTTGCTTTTCATTCCTTCTCTCTGATGATAATTACCTGATTTGGTTGTGCAGACTTTTT
AGTTTCACCCGCAATTGACCAAATAAATGTAATAAACCAACCAATAATTGACCAGTTAAA
CAGTAAAGATGTGAAAAAGATTCCTACTGTCGATTTTGACCCACGCATCAAGGCGATAAA
CCATGGAAGCATGTATATAATAATAGCCAACACACCTGAAACTAAAACCATAAAAATTGA
ACCTGCTACTAAAGTTTCCATGTTTTCCTCACTTAGTCAAATTTTTTACACATGAATTAT
AAGAATTCACTACATACTCCATCGGAGCATTTTTACCAGTACGCCATTGGTAATTATTAG
CCCAATTAGCCCAAATTTCGGCGCAGTAGTTTTCAATTTTTTCTTCGCGTGTAATTACAT
CCGAATTACGATATGCTTGAGCAGATTCATCTGGACGAATAGCTTCGTCAAAATTCGCCT
GCATTTGTTCGACTGTTTGCTTTGGAGCTTCTTTATAACACTTGACATTAGGATTATAAA
ATTTGCTTGAACAGTTTACAATTTTTCCTACATCAGACTGATTTACTACCGGTCCTTGAG
CTACACAACCAGTAAGACCTAATGCAATAACCAAAATAGCGATTTTCATAATAATTTCCT
CAAATGCAAGTAGTAATTACTCCAGTAGTGCTTATGCAGGTATTACCCATTTGCACACCT
AAAGATCCATTTGTATTCACATGAAGATTATCATCAATTTTAACTGATATTTTACCATTA
GTGTGAATAACGGTGCTAGATTCAATCACAGGTTGAGTATCATTTTCAACACTAACAAAT
GGAACTGCTGCTAATGCAACTAAACACATTGCTAAGCAACCATAAACAATTTTCATTTTA
TTCTCCAAATCCGTATTAGTTGATAGTTGTATAGTACCACAGAGGAACAGTCTTGTAAAC
AGTTTTGTGAAAAATTTTTTAGGGAATTCTAAAGGTCCAGAATCATCTGTTTTTCATAAG
TATAGATTTATATTACTTGTATGAAAAAGGGACCCGGAGGTCCCTAGATTTATTCTATCA
ACCAAACAGGAAGTCTAACGAAGCTTTTTCTTCATAGTCCATACCAGCCGATTCACACAT
ACCCGCAAGCGGTTTAACAAACGATTTTTGGAACAAAGTTGAGTAGTCAATCCAAGACAG
TACATCAGAACGAATTTCTTTTGGAAGTTCTGTGCCCGACGGCCAAGCAATGCACTTGTC
ACCAAATGGATTTCCTTCACGCAATGGAAGAACCATTACCTTATTTCCATCCAAAATTGG
AGCTACACCTAAACCGCTAACAGCTCGACGATAAGTTAGCACACCACGAATATGGAACGG
GCATTTAAATCCTGGCCAACCTTTATCATCATATTTCGCTATATCGTTCGCAGTTTTTAC
TTCAGCAATAACTTTATAATCAAGTTGACGATATTCTTTCTCAAAGTTCTTGTAGTATTC
TTGGACAGACTCTTCACCTTCCTGAAGAATACGACGAATACTTTCTTCGAGAGCTTCTTG
CACTGCTTTTGGTGTTGAACTCTGCTGAGTTTCCATACCCATGATTTTTAGATGTGGTTC
AGCAAATCGCTTATCTTCCATATCATAAACGTTCAGAGCATAACGCTTTTTCGCTTTCCA
AAATCCACCAACGCCCTTTGAACCAAGTGGAGGGCAAGAAATAGCTTCACGGTCCATATG
CATCAGATGCTCGCGGTTATTCATATAATCACATAACTCACGATATGCAACATCAATCAT
AGGTTCCATCTTTTTCTTACCGAACTGATTCATGAATTCAACTAAATCATTTTGTTCTTT
AAAGCGGTCAAGACCGACTTTTTCAATAACTTTATCTACACAAACATATACCGAATCAGT
ATCACCTGCTGCAATGAAATCTTCACCATTAGTTCCGCATACTTTATTCAGATATTCATT
AATTTTACGAGCAATCCACTGAATACCGACTTGACCAAAAATTGTGATAGCAGTAGCATT
TCGCAAATCATAGTAACGGAAATGAATATTACCAAGAGCACCATAAAGACTGTTAATAAG
AATTTTACGGTTCAGCTGATTTGTGTTAGCAAGTGTAGCCGCTTTTTCACATTCTTCAAT
CAAACTATTGAGAACAGATTCAGTATAATTCGATAGTTCATTTAAGAAATCATCACTGAA
CTTAACATATCGTTCAACTTCAGGTTTAGTTGAACAAGACCCTGCGCCTTTCATAATAAT
CTTTTTAATAGCTTCGGCATTCATTTCTTCAGCGAACATTTTCTTTTTCCAATCTTTACG
CTGGAAAAATACTTTAGCGATTTCCTTTGGAATGATACCTTCTTGATGCTTATCATACAT
CCATCCATTCGGAGAACAAGAATATTCATCACTCGGTTTAGGAGCTGTTCCTGCGATATA
TTCATGAATTGGATGAACTTTAAACTGACCACGAATAGTCTCAGGACTAATGTTAACCTG
ACGAATAATGCTCGGATACAGAGACGTCAAGTCAAAACTCATAATATATCGACGAGCAAT
TGGTTTAGGTTCAAATACGAATGCACCCGGAAAACTCTGTTTAACGTGCGAACCTTGTTG
AGGAATAACCTTATGTTCACCTTTCAATGAGTTAAAAATAATAGCATCCCAGGTTTTAAT
AGGACTCATTACACCGGAAAAAGGCATTTTAGCGTAATAAGACATACTTAAAACTAGATC
GATAAACCCGCGAATTTTATCAATTGCTTGAACTGATTCTACGTCAATGATGTTATAACT
AATGTATCGTTGATGATTAGTCTCACGAAGTTTATTAATAGGACCGTCGTATGGTAATTT
ACCTTTTTTGGTTTCATGCTGAGCAACTGATTCCAAAGAGAATGACGGCAAATTAGTAAA
AGCGAATTTCTTGTACAAATCTAAATAATCAAGAATAGATACGCCATCAATAGAATAAAT
TTCTTTGCTGCCGTACATATTTTGAATTAGTTTAGATTTTACTCGACCGATTGGAGAGAA
GCGTTTCATACTACGTTCACCGAGAACCATTTTAACGCGATTCATGATATACGGAACGTC
AAACCCCTCAATATTCCAACCAGTAAAAATAGCAGGTCGTTTCTGTTCCCAGAGATTGAT
ATATTCCATGAGCATATCACGCTCATTATCGAATGGCATATAAATTACTCGGTCAAGAAT
TTCTTGAGGAACTTCATCACCACCTTCACAGTCAAGCTTAGCAGCTAACTTTGCATCCCA
TTTTGATACTGAACCGTACATTGAATTCAAAAGGTCGAAAACATAAAAACGATCATCAAT
TGAATCGTAATGAGTGATAGCATCAATTTCATATTCAGCTTTCATTGGGTCAGGAAATTT
ATCACCAGTAACCTCAATGTCACAGTTAGCTACACGAACAAATTTTCGGTCATAAACAAT
TTCTGAACCGTATGTATCACTGATATAAGCGAGTTTAAAATCGTTCATACCGAGAGCTTC
GAGACCGATGTCTTCCATTCGTTTCATCCAATCTCGGGCATCTTTCATTGATGGAAATTT
TTGAGGAGCACAGTTTTTACCGTAAATGTCTTTGTATTTTGATTCTTCCTTACAGTGTCT
AAACATAGTTGGAAGATATTCTACTTCACGAGTACGTTCCTTTCCATTTTCATCAATATA
ACGTTCAACAATGTTATTTCCGACTGTTTCAATAGAGATATAAAATTCTTTCATAGATAT
TCCTTAGTTTATAGCCCGAGTTATTAGGCTCTTGATATATTATACTCCAAATAAGGGGCC
GAAGCCCCTTTGCTTTAAATTTCTATTGTACGACGCGATATAACGGACTTGCCATCTTTA
TTTGTGGTTTTGATAATCGCATGAAATGCGCGTTGGCGTGGGTAAACTAATCGTTGGAAT
TCTTTTTGGAATTCTATTTGTTTATCTTCCGGGAGGTCAAAAACCAGTTGGGTAGAAGAC
AGACCATATGTTTGTTTTAGATTTTCGAATATTAGAAAAGCTTCGGTAAACTGAATCTCT
GATTGTGTCATATAATTTACCTTAATTACCAATCGTATATTTAGGAACGAGTTTCCATTC
ATGTTTTTGTTTAAAAGAAATAACTCGGAAGTTATTAGTTAAATCTTTCATAAAAGTTCT
TTGTCCAGGAACGATTTCAATCAGTCCCCAATCTTCTAACAGCCATGCAATCGAATCACG
ACGAACTTCATCTTCTTCTGTCATTTCAACTTGACGGCCATCCATACGAAGCATTTCTTT
AAAATGAACGATATAGTATAGTCCTTTTTTCTGAAGAATATGACAGGACTGATACAGAAC
TTTATCTTTATTATTAGCAATTCCCATACGAGTCAAAGTTTCTTTTACTTTCAGAAAATC
TTCAGGTTTTTTAAGAGTAATTTCAATCATTTTACCATTCCAATGCTAGTTTTTTGAGTT
GTTTCTGTTCTTTTACGTTCTTAGTCACTTCTTTTAAAAAATCATCCGTGACTAATCCTT
TAAGTTCTTTTAATACTAAAGGTAGTTTTCCATTTTTAGTAAGAATTGATTTATAGTTAA
TTGCATCATTTGTATTAACTTGATACCGCTTAGCAAGTAACTTAATAATCAATACTTCGG
TGGAATCTTCAACTAGTTTTGCCCATTTACCATATCTTTTACCACGAGGAACTGCAGCCA
TTAGATAATTAAAATGAGCTTCATCACTTAAGCCTGATCCAATTAAATTCATAGCATATA
CAGCTGGCATGCACTCTGGAAATTGTGATAATGCATTTTCAACCATGAATTTTGAATAAT
CTTTTTGAGCAATAGAGCATTTAGTTTTATTATTAATAGCTCCAATTATTTCAAAAAATT
CATTTTCAGCTTTTTCTTTAAAAGTATCAGCAGCAGATTGGACAGCTGTCCAATCTTTAG
AATACCAAGCAACTTGATGCTCGTTTAATTGAATATCATCTTCAAATAAGCTCATATCAC
TTCCACTGCATTTCGCATGCTAATTGAATGAAAAGATAAGCTAAATGCAATTCAGTATTA
GCTGCAATACCATGATACTGATTATTTTCGCCGACAATTTCGTACATACGAATAATACTC
TGTGGTGTTACACGTGAATAGATTTCTTCGGCAAGTTTACCAATAAACCAAGAATAATCT
GCTGCATATTTTGGTGCTAAAGCTCTGAGTTGTTTAACATCTTTATTTTTGAGAGACTCA
AGAACATCATCAATAGCACCACGATCGTTAGTAACCAGTGATAAAATACCAGCATCCAAA
ACACCTTTAGATGAATAACTATCGAGCTCGCCAATAGTTTTACGAAAATCAGGAAAATTC
TTTTTAACCAAAGCTGCTACAACTTTCATATCAGCTATAGCAATACCTTCATGTTTACAG
ATTTCAGTCAATCGACGAATCATCTGCTTCATCATTTCAATTTTATCTTCATCGGTCGGT
TGACCGAATGTAATAACTCGACAGCGTGACTGAAGTGGTTTAATAATACCATCAATATTG
TTAGCAGTAATAATAATACTACAGTTTGAACTATAAGCTTCCATAAAGGAACGAAGATGT
CGCTGAGACTCTGCTAACCCTGAGCGGTCAAATTCATCAATAACGATTACTTTTTGACGA
CCATCAAATGAAGCGGCGCTGGCAAAATTAGTCAAAGGACCACGAACGAAATCAATTTTA
CAATCTGATCCATTCACAAACATCATATCAGCATTTACATCATGACACAATGCTTTTGCT
ACAGTTGTTTTACCTGTTCCTGGAGAAGGAGAATGAAGAATAATATGTGGAATCTTACCT
TTACTTGTAATAGATTTAAAGGTTTCTTTATCAAAGGCGGGAAGAATACATTCATCGATA
GTAGATGGACGATATTTCTGTTCAAGAATGTGTTCTTTTTCATTTACGGTAATCATAATT
TCCTCATTCAAGTTTTAGTGTAAATTATAAAGGGCCGAAGCCCTCTATTAAAAATCGTGG
GTAGAATCAGCTTCAAGAGCTACTACATAATTCGCGTGTTCACCTTCAAATTTAGCAGCG
CCTTGTTTACCTTTTGCCCAGAGCAGAAGTTTATAATTTCCTGGTTGCATTTTCATATTT
GCCATATTGATAATGAAATTAAATGTATTTTCACCATCATAATCACCAAGAGTCAAAGAA
TATTTAACACGGGTAAGAGCAGAATCTTCTACTTTATTAAAACCATTAATTACGATTTTA
CCTTCTTTTACCGTGATAGCAATTGTATCAATTTGCAGACCACGAGATACACGCAACAGT
TGTTGAAGGTCTTCAGCTTTAATTTCAGTAACAACAGATGCTACCGGGAATGGAATTGGT
TTATTAGGAGCAACTACTGTACTCGGATCTGCTGCTGGCCAAAAAATTGTTGAGCGGGCA
TCAGCAATTTTAATATTTCCATCTTCTGACTGGGAAATTTCTGCATCATCATTAACTAGA
GACAGAATACCGAGAAAACCGTTCAAATCGTAAATTGCTACATCAAAATCAATAACGTCA
GAAATATTTGCTTCCGCATAAGTTGTACCATTAACCGCACGAGTCATAATAAATTGACCG
GATTTAAGCATAATACCGGAGTTAATAGTAGCGAAATTTTTAAGCAGAGCAGTAGTATCT
TTAGACAGTTTCATGTAATTTCCTTCAATTCAAATGAGATTTAATTTTATAACTAATTTA
ATAAAGCAATTAACGATTAAAATCAGCCGCAATTGTTTCAGCAACAATTTGAGCAGCAAC
AATTAGGCGTTCATCTGCATTACCGCAATATTCATCTTCAAGGCGTTCACCACATGAAGT
CATAATAAATTTAGCACCGGCGTTTAGGGATTCTGCAGTATGTTTGCGCATTAGTTCAAT
CCATTTATTACTTACTTCACGATCGATAGCTTCATAATACGCATGACGAGCAGGTGCAGA
TTTAATTTTGTTCTGAATAACTTCCATTGCGTTATCAGAAAGAGACAAAACCCATGCGCG
ACGAATTTTATTTTGGTTTTGCGGATTTGAGTCAGAACGCACGTGTTTTGGCTGAATATC
TTTTACATCAACAGTATAATTCACAGTAATTTTAGTCATAATACACCTTTAGTCATAATA
ATCAGTAACAGTCCAAGCTTCATTTCTATTGGACATTATTTTTGTATATTCTGCTTTAAA
TGCATTCCTAAGCATAGATTCAGTAACTATATGCTCTTCATTAGAAAAATTATTTCTCAG
AATATATCGTTTTATTTCAGGAAAAGTTAATAGATGCTGTCCAGTTGAATATTCCATGTT
TTTCCTCCATAGAGATTATACTCTAATAAATTAAAGCATAATCTCTTATAAATTAAACCA
TTACAGTAAATCGACCAACTTTCTTCATTTGAAGATGCTGACCATATGCTTGTGGGTCAT
GGTCTCTATGTGAAATAACAAAAACATTAGTATTTTTTAAACTATCTAAAATAGTTGAAA
TAGCTTTTACACCTTCAACGTCGGTCGCTGAATCAAAAACTTCATCAAGAATTAGTGTGT
TTATTTTAACACCTGAAACTTTTTCAGCAATATCACGCCAAGTAAATAAAAGAGCAATAT
CAATTCGTGCTTTTTCACCTTGACTGAATGAAGCATAACTAAAATCTTCACGACCACGGG
ATTTAATTGTCTCATTAAATTCTTCATCTAATGTAAACACATAATCCGCTTCCATTATTT
TAAGATAATGGTTAATCTGCTTATTAAATAATGGAATGTACTTTTTAATAATAGCACCTT
TAATACCAGAATCTTTGAGCATATCAGTCAAAATTCCTCGGTGGTATTTTTCCATTACTA
AATTAGTTTTTGTCTTAACAATTTTATCAAGTTCTTCTTGAAGCAGTGCTATTTCATCAG
CATGGTCAATAAACTCAGAAGATGCTTTTTCTATAGCCGCTTTAACTTTTTTAGCTTTAT
CTACTGTCGTGATTAGAGATTGCTTTTTATTGCGAATATCATTTGCCAACGACTGCTGGG
TTTTAATATTATCTCGGTATTCATCAACAAGAACTTTTAAATTATCACGGTGTGTTGAAA
GCTGTTCAAACGAATGCGTGCATTCAGAAACTTTATCTTTAATTTTAGAAACAACTTTAT
CACCGGAACTCAATTGTGACAAACAGGTTGGACATAATCCACCTTCGTGATACATATTAA
TGACTTTATTATACGAGTCAATTTTTGATTTAATTAAAACTGCTTCTTGACCGATTTTAT
TAAATGCATCAGTCGGGTCTTCGTCTAAAACAATATTAACTAATCTTTCATTAGCTTCTT
CTATTTCCGATTTTAGCGTTCTAGCTTCTTTTGCCAAATCATCATACATATTTTGCAGAC
GAGTAAGATTGTCACCCGTTAATTTTTTCTGGCGTTCAACGTTATCATTATATATTTTAA
TTTGTTGGATAATACTATCTTTTTTAACATCAAGCACTTGGTTTTGTGAATTTAATTCAC
GTATTAGTGCTTTATTAAGCTTATCCATTTCAGCTAATGTTCCTACCTCAAGCAGGTCTT
CCACAAGCTTTCTTCGTGCAGGGGTCGACAAACCCATGAAAGGGGTATACCCTGCTGTAC
CAAGGACAACAATCTGCTTGAAACTGGCATATGACATTCCGATAAGCTGTTCAAATTCTG
CTTGGAAATCTTTACTGCTGGCAGATTCATTAAGACGTGTACCGTTAACGGTGATTTCGA
AAACGTTTGGTTTTTGTCCTCTTTTGATATAGTACTTTTTCTCATCATATTCCATCCACA
GTTCAACTAAAAGTTCTTTCTTATTTGTGCTGTTTATTAATTGACCTTTCTTTACATCAC
GAAATGGTTTGCCAAAAAGCCCAAATGTGATGGCTTCTAACATAGTAGACTTACCACCGC
CATTTCGTCCAGTAATAAGAGTTTTTTGAACCTTATCTAATTGAATGTCAATAGGATTTC
CACCTACTGACATTATATTTTGATACCTAACTCGGTTTAGTTTAAAATTCTTCACAAAAG
ATTCCTTTTAATGTATCTTTTAGACCATTCTATCATATCATCATAATCTAAAAAGTATTC
ATCAAATTCAGCCATGCAAACAACACCTTGTGCTGCTTTTGATGTGATATAAATTATTCC
AACATATCTAGAATCTTCTTCGGTGTAATCAATATTTGCTATGAATTCATCATTAATGTC
AAATGTCGAAAACTTCACAGTATGCATCCTTAATACAAGATACAGCCATATCTTGTAATG
ATTTAGGTGTGTCATTATCTAAAATGTTGAAGTTAAAAGATACAGCCCAGTCAGTGCAGA
CTGTAACCTTTTTAATACAATTATCTTCAACCTCTAACGGTTCAAACCAATATTCAATAA
TTTCTTTATGCCCAATATCATCTTTTACTTCGCATTCAAAATGCTGACTCATCATAACAT
TTTTAAATTCATCAAAAGTCATTGTGTTGCCTCTACATATAGCTGGTTTGCATATTGAAT
AAGTGCTTCACGGTCAGAATCAGTGATGTCTGGAATTGCATTAATGTATTCTTCCATCAA
CGTCTGAAGAGATTGAACTTCAACTTCTTCACTGTCATCTGACTCGACAGAGTTATCAAT
TTTTGACACAACTCGTAATGAATGCACAACTTTTTCTAGTTCAGATTCGAACTTCGTCAG
ATTTTTGTCTACTTCAGTTACTATAACACGTACTGATAGATTTGTAAAATCTTTATAGTC
AATTTTTCCTTTAAACGGATAATGAATTCTACGATGCCAGGTAGTATTGTTTGGAATAAA
TTCCATTCGTTCTGTTTCTGTATCAAACATCCAGAACCCGCGAGGGTCATTCTCGTCACC
TGCAGTTAGTGTCCATGGTGTCCCAATATATCTGACGTTAGCAGCCTCAGAGATAGTATG
GAAGTGACCAGACCACACTTCTTTATAAGTCTTAAGGAAATCAGGTTCAAGACCGTGAGA
TTTCATTCCTTTATAAAAATAAAATCCATTCAGTTCCCAGTGACCAACACAAAAAGAAGC
AGATGAAGTTTTGATGTGTTCAAGAATTTCACCAGTATTTTCTTCGCACATCCAAGGAAT
CAAATCAATCAAACACCCGTCAAAATCTACCGTAGTAGGCTTATCATATACTTTAACATT
AGGATATTTAGCCAAAAGCTCAGTAGAGGCATTTGGAGTTAAAGTATTTTTAAAGTGCAT
ATCATGGTTTCCTACAACAGTATGTAGGGTAATACCAGCATCATCAAGCATTTGAACTAT
TTCACGAGCGAACTCCATAGTTTTATGCGTGATTGCTTTTCGCACATCAAAAATATCACC
ATATTGAATCCAGGTAGTAATTCCATTTTTCTTAGAATATTCTATTGCTTGTTTAATTCC
ATCCAACTGAATGGACTGAACCCACTCATCATCAGCTTTAACGCCTAAATGCCAATCACC
TAAATTTAAAATTTTCATATATCAAGAACCGTCATTGAAATGCAAAATAAAATTATTGAA
ATAAATCCATCTGGAGTACTAAAGAATCCAATCCAACATGCTCTAGTGAATAGATAAAAT
GCAAGAAAAAGTATCACGTATCCAAGAAATATCATTATATCAAACTCCGTATAAAGCTAA
AGGGCCGAAGCCCTTTATTTTGTAATAATGTCAAACTGTTCTTTAAAGCAGAAGCTTGAA
TCTTGATGCTGATACAAAAATTCATAAGCTTTTTCACGTTCGCGGTCATAAAGAGTTCGG
TCAGATGACAGTTCTTTAATACGTTCAAATGTTGATTCCATATCATTTTCATCAAACCAA
ATGATACCGCTATCATGCGAGGTCAAAGGAGTATTATCAACACGGAATTTTAAATTTTCG
CCAGTAGATTTCCAAAATACCGGAATTGTTCCGCATGCACCAAGCTCGAGATGAGTATAT
TCTAAAGAACGCTGTAGATATTTTTTGTCCAACTTACTCAGCTGATAACCAAATCCGGAT
TTACTCATACGTTCAAGCATTTCGCTATTTACATAACGGTCAAGAATTTGCGTTGGCAAA
TTAGGAGCAATTTTAATTTGGTCTACTTGATGAAGACGATAATACTCGTATGGAATTCCT
TTTTCTTTAATAGGAATGAACGCTGGAGAACGTTCCAGACCTTCCATAATAGTACTTAGT
CCTGCAGGTTTAAGATGTTTTTCGTGAAAATCAAACATCTGATAAAAACCTTTCCATGTA
GTCGTACGACCAATCCAACGGTTGATATTCATGTTAATTTCAGAAACATCTTTCCAGTAG
GTTGATCGAACCTTCGCAATATCCATAGGAGGCTGAAAGTTATATACGGTCGGTGCTTCT
TCAATATCATCAAACAGAGAAACAGTTTCTGGATACCACTCTTTCATCAGAACTTTATTA
AAATCACCATTATCAGAATGGCTAAAAATAACATCAGCTCGACGAACAGTTTCTTCTAAT
CCCAAATTTCGACGCAAAGAAAGAGAAGAATGGTCATGTTGATAAACTACAACACGAACA
GAAGGTTTAATGTTATCAATAATTTTTTTATAGTTATTAATCGTGTCTTCTTCAACCGAA
GAAGCAGGAACTGAATTGATAATTAGAATATCACAATCATTTACCAGCTTAAGTGTTTTA
TCATATTCTTTTGCCAATAAAACCGGAATTGAAAATGATTTATAATCATGCGCGCAATTA
CGAGTAAATGATTTATCTTTAGCATAAACCAAAGTTACTTCATGACCATTTTTAATAAAC
CAATCACGTTGCTCGAGTGAGAATTTTGTTACACCACAACCTTCAAGACCTCGAGCCATA
AAAATACATACTTTCATTTAATATCCTCTTTGTTTTGGTTTATTTTACCAAAAATTTATA
AAGCAATATAGGAGCCGAAGCTCCTATCCACATAATACGCCATACAGAGGCTCATTAGAA
CTTTTAAATTTTATGCGCTTATATTTTATAGTTCCTTCTGCTTTAGCTTTATCATGAGAC
TCTTTAAAGAGTCTCATCATTTCCTCTTTAGAGGAACGAATTTTATTATAATCTATTTTT
ATAATCTATTTCAGAAGTCTGGGTGTTCATCTTTCATAACTGCCACCATTTTTTGACTTG
ATAAGAATCAACCCACACTTTCATATTAGGGTCTGCTCTTACAATAGGAGGATTAACTTC
TTTAATTGAACTATCATGGTATTCTTTTTCAGAAATTTTCACACAAAGATGACCATTCAA
AGATTCAGTAAATCCTGCATTAATTTTAAGACGCTTGAATTTTACGTGTGTAAGCATCAA
TCATATCCTCAATTTGCGATCTAGTAGTCTTCCAAAGAATACTGATGAGTTCATCGTTAT
ATGGCTGTTTTAGAATATCCCGACTTTTCTTGATAGCATATTCGTATTGAGCAAAATTAT
TGTTTTCAGCTGCGATCTGAGCGTGCTTATAAAGACGATTCAGTTCGCGTTTGTTTTTAG
ACAATAACTTATTTGCTTTTCTTTCTGCTTCAAGACGGTTCTTTTCTTCAATAGAAGAAA
TAAGCTTTTCCACTTCATCATTAATTTCGGGTTTATCAGTCATATTATTTCTCTAATATA
AAATAAAAATCATCATCTGTTAAATGATACCTGTAATTTAATTCTACACCATTAGATTTA
AAAGCGGTATCATACGGATTTTCTGGATCAATATCAATGTCAAGAGCTAAAACTTCCCTG
AGATACATTTTAAGTAAATAGGGAATAGCTTCAACTTCAGGTATTTCTTCCAAGAATCCG
GAGAGGTTAATCGTTAGCCTCATATAAAAAATCCAAACTAGGAGAATCGTCTACAACACT
TTTCTTTTCAGCCCCCGGTGTTCTATAGGTTGATTCTTCGTAATGCGTCATTTTATCGTA
GATGTCTTGAATAAAAGTTTCATCTACTAACGCAACCATATCGTCGTCACGACTGTCATA
GACATTGTGAACGAAGTAACTATATTTCTTTGCAACTTCCTTACGTTCTTTTTTAATACG
TTGGACGAATGCATTAAAACAAGCTTGAGTTATATATGCATGCGGGTTTTTATATTTCGT
TTCATCAAAATTGTGAAGCCCCTTAATAGAAGCTTCTATACCATCTGCAATCATTTCTTG
TTTCCAAGACTGGGTGTATCCTGAAAAGTTGAAACGTTTAGATAAGCCTTCTGCAATAAG
CATAATGGCTAATCCGATAGTATCATTTTGACGAACTACTTTATTTGGGTCTTTATTATT
TGCTAATTCTGTTTTCCAATCAATAATAGCTTGTAAAAGCTCTTTATTGTTTACGTAATT
ATATTTAGGCTTAGTTTCTGACATTTTCACCTCTTAGCTCAATTCATAGATCTATTATAT
CATAATATTTGAAGATCTATCTTAAAGCATAGAGAATATCAGTTATCTAAGTAAACAGTA
TGCTTGGAATACTTTCTCCATCATCTTTTTAAATTCATGAATATCTATATAATGACTAGT
TAGATCAGCATGATATAAAAAGTCTAAAAACTTTTCATGTGTACTAACATATTCACTTTC
ATTTAATTCAGAAACATGGCTTTCCCAATATCCAACACCAGTCTTATTTGGGAAAAATGA
AATTTTCATGTATTGGTCAACATAGAAAATGCCATAATTTTCAATAGTAATAGAAATATT
AATAAATTTCATACATTCATCAACTTAGCCGCTTCAAGAGCTGCATCTAGTGAATCAAAT
TGGTCAACATATTCAATCAATTCGCCGTAATTGGCATATAACCACCATTGGCTAAATTCA
TACTCAAGGATGAATCCATTTCCTTCAATTTGAGTTAAACCATTGCCGTTTGTATTTACT
TCATATCCGGCAAGACGCAAATCGTTAATAAGAGCTTCGTTCATAATTATACCTTAGTAA
TTTTCAGGTCTGCAAATTTTTTCTTGCGTTGATTTTTCATGCGACGAATAGTTTTATCGG
AAATTTCATGCTTTTGATAAGCTTTAGATTCTACACCAAAAGCTTTAACATCAAATTCTG
ACAAGATATATTGAACCAACAATTCACGGACAGTATTGCGTCCAATCTTCTGATCATTCT
GTTTCATCGTCTGATGAAGCTCTTTTTCCCATTTATCCAAAATTTGAGGAGTTACAATAT
CGCCTTTTTCTAGCAAAGAAACTACTTTATCATATGCGTAAGAATTGATGGCGTTTTTAA
TTTGAATAGTCATACATTATCCTCAATTGCATTAAAATTTTATTATCCAAAAAGGGCCGA
AGCCCTTAGCTAAACTTTTTGGCACCCTTCCAGCCTTCATACATCATTGCGGCTGATATT
AATATTGCTCCTGAGCAACTTGAATATTTGTGATTCCAAAACCAGTTTAGAAAAACTTCG
TCATCAATACCTTGTGCTATCATATTTTCAAAGAACTGACGACGAGCTTCGGCAATTCGC
TGCGATAATTTAGATTCAGGATTCATTTAAATTTTCCAATTACCGTTTTCATCAATAAAT
TTAATCCAGTCATTTACTGACCACTTAGTTGTATCGCCTTTTGGAGTAACATTTAAAGTA
TATAGCCCTTGCTTAAAAAGCATTCGTTTGATATTCATATTTTCCTCAGCTGTAACGATA
ACACTCGTTTGATTTACGTTTAGCAACTCGTTGAGAAGTATTATAATCAAAATCATCGTC
AATGTAAACTGATTTTTTCAACTTTCTTACTTCACCACGTAATTGGCGATTTAGCTCATC
TTTAACTTCTGAATCAATACCTTTCATTCTACGCCATTTATCTGAGCGAAAAATGTTTTC
AACCATATCTTTATGACTTACCCCATCAGGAGCTTTACGCTTTCCGAAATAGTCATAATC
ACGAACTTTCAAGTCTTTACGACGATACGTTTTACCCATGGAGTTTAATTTCCTTAGCAA
CTGAACTAAATACAGCACGATCACAAATCATGTGTTTATGTAACTTGAGTATAATATAGC
AAACTTCAAAACTATTTACATGAGTAACACGAACAGCATTACCATTAATGAGATAATACT
GTCCCTTTTTAATTTCTTTATCAACCACAACCATATCAATTCCTCAAAGGTAATTCATGT
GTTAATAATACCACAGTTTGAGCTCGTTGTAAACAACTTTGTGAAAAATATTTCAGGGAA
TGATAAGAAAGGAACGATAGCTTAGAATGGTAATATACAGAATGTAAGAAAGATGGGCCC
AGAGGGCCCATCTTAGTCTTCTATGATATCTCTATCATATCCAAGTGAAATGAGAGTTTC
TTTGAAGTGTTTAATGTTCTTTTGTCTGGAATCATTAATGAAAATGACTGGATAACGAAT
GTTAAGAGATGTGAATCCAGCGCGTTTAGCAAGAGATACAATCAGTTGACGATCATACTC
AATCTTACCATTATTAGTAAGAACTTTATAAAAAGTAAAAGAAGCATTGAGCTCCTTTAG
AAGTTTTGTAACTGATTGACATCCAGGACAACGACCTACTTCATCTGGAATTCCATAGAC
TTCAATCTTATTCTTTAAGTTCGAGTTTTGTTCCACGAGAAATAATTCCTTGGTAAGCCC
AATAAGGAGGATTAACAGAATCCTCACCAGAGTTTTCTTCAGGAAAATAAACCTGGATAA
CAAATCCAGATGCATCAAAGGTAAAATAAACCGTAGGCAGTTCACCTAACAATTCGGCAC
GAGCATTAATTTCTTCGAGATCTCGTTCACGTCCAGCCCAAAAACTAGGATTTAGACTAC
ATTCGTAAAAATAATAGTCATTACCGAACATATCAAAACCATTCCAACTCTCTACTACAT
ACCGCTCAAAACTAATCATAATTAATCCTTTTTATCAAGAACCGCATTCAGCTTGTTAGT
AATTTTATCCAGACGCTCATTGAACTCACTAGAAGATAAGCCCTTTTCTGGAGAAATTAA
ACTAATCACGAAAAATATAGCAATAAAAGGAATAAGGAAAATAGCTCCAACTGCCATAAA
CAGAAAGAATGTTACAGTTGTAAGAAAATCAGCTAAACCTTTACGAAATTTATACATATT
TTACCCTTAATTGATTAACCAAGCATTGATAAGCACTAAACTATATTGCGAATAAAATTC
TGGACCAAAATGAAAAATCATATCATTTATAGTGTCCATAATATAATTCAATTTAATCAT
GTTTCCACACCCCATCGGTATTTGACCAAAGTCGCTGATTATCTGATCCTCGCCACAGCT
TTTTAGTCGGAAGATTTTTCTCATACTTCCCATCAATAATAACATCAACATATTTAAGCA
TTTCTAGTTGTTTAATATCTTCAAACTTATATCCTGTCCACAACCAAATGCTTTTATTGG
GATAAAGATTTTTTACTGTTTGAACCACAGAGTGAATCACGTCTCTGTTATCAGGATAAA
GAGGGTCACCTCCGGTTATAGTCAATCCTTCTATATAATCATTATTCAAACATTCAATTA
ATTGTTCTAGTGTTTCACCAGTGAATGGAACACCATTTCTAGCATTCCATGTTGATTTAT
TATAACACCCTTCGCATTTATGCAAACAACCTGTAACGAAAAGAACGACCCTGCATCCAG
GGCCATTAACAAAGCAGCACGGGTATATTCTGTCATAATTCATAATAATATCCAATTTTT
CCAAGCTTCATCAGTAGAATTACAGCGTTTTAGTATAAGATGTCTAGAAACTTTTATGGC
ATTTGAGCACTTTTTAATTGAATCATATTTAATACCGTCAACTGAAACGGTTTTGCTAGT
TGGAGAAATTTTTCCATACATTGGATTTTTTGAACCAACTTGAAGTCCTTTTAAAGCTTT
TGAAAGCTTTTGTCTAGATTCCAATGACATGGGAGAATTCTTTTTACCTTCAATAATTCG
CTTGCGATGAAGTTCGCGTTCTTCTTCACTCAAAGAATTGTAATATTTTTTAACACCGTT
TGATATAGATAATTTATCTTTCTCAGACCGCTCTCCAGCTAATTTACTTCTAACATCTTT
TCGCTTCATAGCTATGCTTACGGCTTTATTTAAATTTGCTTGATGCTCTGGCGTACGTTT
ATATTTCTTTAAAGCACGTGAAATAGCTATTTTAGTTTCTTCTGTTCTCTTTTTACCTTT
ATTTGCTAAACTATTTTTGTGGCGATGTTCAAGTGTCTGTTCATAGGCAGTTCCACCTTT
TCCACCAGGTTTAATATTATATGTATTTTCTTTTAGACAATACTCTTCAGTTACAAGTTC
AGCTTCTTTATCATACATTTGCTTTTCATTATCAAAAACATATAAAATTTCTTTATAAAA
ATTTTTTCTACCATATTTTAATAAAGCAGCACGAATCAATGTTCCTGATCCCATATAACC
GTCATTTATGTTATCGGTGCTATGCGCACCGATATATTCCTTTGAATTAATTATATTTGT
TATTTTATAAATTATATAATACCTATGCATGTTTTACTCGTTTTACCATTTCTTTCATTT
TACCTAAATTCCATGGAACAACGCTAGGAGTTCCAAGATATCCGCATGTTCTTCTTATGG
TGTTCATCTTTTTAGGATCAGTTTCTCCACAAATAGAACAAACAAATCCGTTTTCAGTAG
GAGTCATTTCATGGGTACTTCCACATGTAAAACATTTATCTACTGGCATATTAACACCAA
AATAATCTAAATGTTGTGCAGCATAATCCCATACGGCCTCAAGACCCTTTAAGTTATTTT
TCATATCAGGAAGTTCAACATAAGAAATGTGACCACCTGTCGCAATGAAATGATATGGCG
CTTCACGAGAAATCTTTTCAAACGGAGTAATATTTTCTTCTACTGAAACGTGGAAACTGT
TAGTGTACCAGCCTTTATCAGTAACATCTTTTACGCTTCCATATTTTTCTGTATCGAGTT
TACAGAAGCGATAGCAAAGGTTTTCAGCAGGAGTCGAATATAAACTAAAAGCAAATCCGG
TTCTTTCAGTCCACTGTTTAAGATGAGCATTCATCTTAGTTAAAATTTCTCGTCCAATAT
CACGACCGACAAGAATATTCAATTCGTGAATACCAATGTATCCTAAAGACACTGAACTTC
TACCGTTTTTAAATAACTCAATTATGTCGTCATCAGGTTTAAGACGGACCCCGAATGCAC
CTTCTTGGTAAAGAATAGGAGCAACAGTCGCTTTAACTCCTTTTAAGGAACTAATTCTAC
ACATCAATGCTTCAAAACACAGATCCATTCGTTCATTAAATAGCTCAACAAATTTCTGTT
CATTGAATTGTGTTCCAATATAAGAATCTAACGCGATACGAGGAAGATTCAGTGTTACAA
CACCGAGATTATTACGTCCATCAAGAATTTCATTGCCAGTCGAATCTTTCCACACACTCA
AGAAACTACGACAACCCATTGGAGAAACAGGAATAGATGAGCCGGTGATAGCTTTATTGT
TCTTAGCTGAAATAATATCAGGATACATTCTTTTGCTTGCGCACTCTAGAGCAAGCTGTT
TAATATCATAGTTCGGATCGTCTTTATAAAGATTAACACCTTCTTCAACAAACATAACAA
GCTTAGGGAAAATAGGAGTTATTCCATCACGACCAAGACCTTTAATACGATTTTTCAGAA
TTGCTTTCTGAATCATTCGTTCAGTCCAGTCAGTTCCCGTACCAAATGTAATTGTTACAA
AAGGCGTTTGTCCGTTTGAACTAAAGAGAGTATTTACTTCATATTCATAAGCTTGGAATG
CATCGTATACATCTTTTTCTGTTTTAGATTGAGCATAATTCAATGCATCAGCGATTTGCC
ATTTTTCTGCATCCTCAGTATGTTTTGCATAGGTGCGTTTAACATAAGGAGAAAGTACTT
TATCTACATTAGCAAAAGTCGTTCCGCCGTATTGGTGGGAAGCAACCTGTGCAGTAATTT
GTGCCATAATTGCAGTAGCAACGCCAATTGATTTAGGAGTTTCAATCTGCGCATTACCTA
GCTTAAATCCGTTCTCAAGCATTCCTTTTAAATCTACTAAACAACAATTAGTAAATGGAA
GAGCAGGGGAATAATCAATATCGTGGAAATGGATAATTCCGCTTTCATGCGCTTTCATAA
TAAAAGACGGGACCATATTTTTGGCAATGTGTTTAGACACAATACCAGCCATAAGGTCCC
GTTGAGTTGGAAAAACACGAGAATCTTTATTAGCATTCTCGTTTAAAAGGTCTTTATTAG
TTTTATGAATTAATCCTTCAATTTCTTTTTCAATTGTCATTTTAAACTCTTTCTAAGCTG
CTTCTTAAATGAAGCTATTAATTGTGTTTTGGTGTCAGATTCATTATATTCAAATCCTCT
TTGAAGCATCTCGGCCATCATTTCCTCTTTTCCTAAACGAGAAAATTCCTTTGATTTATC
TCCAACAAAGTTAGGGTGAATATTATTTTGGGTGTAATCGGATTTTAAATAAGTAAGTAA
ATTTTCTAACCATTCAAGATAATCAACACCTTGTCCCTTTAAGCCAGAACGATTAAATTT
ATGCTTCATTTGACCTTCTGCGGCATTACAGAGATTACAAAGCAATCCACGTACCTTTCC
TGCTTTTGGTCCATTTAATTCATGGTCATGGTCAAGATGATTAGCTTGAACATCAGGATT
TAGTTCTCGTTGGCAAATTAAGCATTTACCGTTTTGTGCATCATAAAATTTCTGTTTTTC
TTCTTTATATAATTTGCCAGTCAATAACATAATAAACCCTTACCTTAAATAGATAAGGGT
ATTTATTATTTTCAAGTATTGTAAAACATTTGATGCAATCGCTTATATTGCTGAATCATT
CGGTCAGAAAAAGAAATTTGAGTTTCAAGCCATTCAATGTATTCTGCGGCAGCTTGCATC
AGGTTACCTTCATAGCCATCGTTATTTTCTTGTGCAGCTAATTTAGCTAATGCGTATGAA
ATACGTTCACCTTGAAAATCGGCTTTAGGCTTCTGAACAACTTGATTAGTTCGCTCTACA
ACTTCTTCAATTTCGCCATTTTCGACTGATTCAGTATTCCACAAACACCAATACGTAATT
GGCTTATCGTAGATGTTAATAATCTTTCCATCAGAAAGTTCAATTTCAATAATTCCAGTA
TCAGGTTCTATATCATCTTCACATTCTTTTGCAAGTTCACGAACTTTAAAGACAGTACCT
GCACTAAGTTCCGGCCAGTAATTACACAGCCCTGTATCAGCACGATTAATTCTAAACCAC
TTATCTACTGTAATCATGTCCCATCTCCATATCAATTAAGTCATTTATCGTTGGTTCATT
ATACACCGTTTCTTCATCAGTGTAAACCGGTTCTTCCGGCTCTGGCTCTACAGTTTCCCA
TCTAGCCGCCCACCAAGGTTTTATAGCGTAATCCATTCTCTTACTGTCTGAGTTACTACA
CGTTCTCGAAGCTCAACCAGTTCAACGGTAGGAACTGCATAATACCAATCAGAATGATAA
GAACCTGACCGAGATTCATTTACCGCTACATGTACATTATGTTTTGGACTAAAGTAAACA
CACTGACGATATTGACATTTACCATCTTGCACCCAGTCTTCTATTTCGACAGGTTTAAGA
TAATCGGAAAAATCGAAATCATAGTTTTCCGAAAATGCATCATGGTCTTCAATAATTTCG
CTCAGAACTGCATCAACATCTAATTTATTTTCAATAGTCATTTTTCACCAATTAATTTCG
CAATTTATTAAGTTCTTCATAATCAATCCAGGATTCTAATCCTGATGGCAAATCAGATTC
AATTTCCCAAAGAAGTTCTTCAATTTTATGAAGACGATCTAGTTCTTCAGCAGGAATAGT
AACCATTGACGGAGCTCTTGCTACATTAATATCGTAAATCATATTTACCCCAGTTTGACC
ATACAATCGCCGTATTTCCACTTAGAAATAGACTTTTCACCGTTAGAATAATAAACTTCA
AGTTTAGCACGATTATTTTTAATTTGAATAACCTTTGCTGTCATCAAAGTTCCATACCCA
TAATAAACTGCAACTTCATCACCTACATATACCGCGCTTCCGCGGTAATCGTGAATATAG
TCAGTTCCTTCGAGCATCATTTAAAATATTCTCGCAGTTGGTCAAATCCACCAATATGAC
TTCCATCAGGAGCAAATACTTGTGGCATTGTCAAACCAATTTGAGTATCACGGCCTAGTT
TAGTCAGAAGCTCAGCGATTTTTTCATCATCAAAAACACCTTTTTCCGGCATAATGTTGA
TAAATTCAAACGGCTGTTTCTTCACAGTCAAAAGACGTTTTGCATTATCGCAATACACAC
ATTTATGGATGTTGCTATCATAACCATATACTTTAAACATATTATTCCTTAATTCCTAGT
ACTTGTTTAAAAGTCTCGTCGTAATCAAGACTTTGGCCCGTTTGTTCTTTATGTTTGTAT
ATAATATCACTTACTTCTGATAGCATATTTTTATATGAACGAGTTAAAGCAGATTTAAGC
ACGCTGTATCTATCAGGAAATTTACCATGTTCATTATAATAAGCTATTGCTAATTCACGG
ACTGCCTTTTCAGCAGTCTCCATATATTCTTTACGCTTGGTCATTTTTAATCCAAAATAT
TATTAGGCATTGGTTGAACACTCACAAGTTCTTTAAAGAACTCAGTAGAGTCAGGCAATT
TAACCATTTCCGGAGCATGCTTACTGAATTTATGTTTACATTGACGGCATTTATATTCAA
GCTCTGGTTTACGCCAGTTAATCAATTGAACTTGTTCAGTACCGCATTCAGGGCAATTAG
GAACGTTTTTAGAAGCTCTTTCACGTCGTTCAACCATAGCCATAATAGCATCCCATGCTG
GAGGTTTAAAATCTAAATCGTCGCAGCCGTGAATCTTTCCACGCATTTCCAAATCGTCTT
CTTCACCAGCCATAATAATTTTAATTAGACTAGAATTACTTGAAGCTGCAATGTCTTCTA
ATAAACGCTTTTTCATTTCAATACCTCAATAGCATTACGTAAACCATTTGCTTTTGCATT
AAGAGCTTTTAACAATTTGGTGTGTTCTGCAATTTGGGCTTCAACTTCAATCAAACGGGC
ATTGAGATATTCACGTTCTTCACTCAAATTATCAACCTTTTCTACTTTAGGCTCTTTTAC
AACTGTCTTATATGTATTAGGGTTCTTCAGTTTAATTAGAATGCGTGAACTGTATAACAT
TGAACCATTATCTTCGGTTTCGTCATACATTTCCACTACGTCCATAATTTTTAACATCAT
GCGGAGTTTATAAGACAACGAACGAATAGTTTTGTTGTGCTTATTAATTTCTTTAGCTGT
CATGGTGCCAAGATTACGCGAGTAATAATAAGGAACCTGGATATTAATTAATACTGATGA
ACCACCTGGCTTGTGTGTTACTTCATCAGCAATAACCCAGTCCTTAACATGAACTGTATT
TGTACGGTCATCGCCCGCGGTAAATAGTTTGCGAGCATGCTTAAAAAGCATATTCACTTG
GTCATTGAATTGACAAGCAAATTTATCAGCAAATTTAGTTTCAGGAATGGTAGAAAACCA
ATCAGCCAAATGAGTCTTTTTAACATCATTAACAATAGTAAATTCAACAAAATCGGTTAT
AGATGTCTCAGATTTAATGAAGCTCGGCTTCATAAAGTTTTTAATGTTCTCATGAGCTAC
AGTAGAAGATAACATACGAGAACGTTTAAACCATTCAAGCAAATTGCCTAACGAATGAGA
ATAAGCGTTTTGAGTATCTTTACAGATATGATTTTCCTGACCAACATATTGAAGGAAATC
ACGAAGAATATTATTGATTACTGCTCTGTGCCCGATCTGATAATTACGGTGTTTAGTAAT
AAGACTGTCAAAATAATCAATATAATGTTTACGAGTTTTCATGTTCTTCTCACTTGGTTA
ATGATTTATACTCCGAGCCATCCTTGGCTTTAAATTTTACTTAATTAACTGTAAAGCCTG
TTCTAAACGATCAAGACGATTAACAGATTCTTCCCAAATCCTTTTAGCCTGCTCATATTC
TTTCTGCGCTTTATTAGAAATTTCTAGAACTTCTTTATAAGCTTTTTCTAATGCAATAAC
TTCTGGACGAATTTCTACCGGTTCAGGTGAATCATCAAGATTATTCATTAGTTCCTCAAG
GGTAGTTTCTTCTTTAGGAGTATTCACAATTTCCTCACATTTTTGTTGATAAATTTCTTT
ATCAGTAGGTGAATAAGCACATTTCACTTCACGAAGACTAATTACAATTTTATATCCTTC
CCATCCGCATATATTCTTAAAAGGATATGTATGAATATTTTCTACTGTTTCCATACAAAG
TAATGCAGTCTCTAACTGACGAGTTATAGTGCTAGCAATTGAGAAAAATTTATTAATATT
CTTTTGGTTAGCGTTTGGTGTAAAAAATCCATAATTCACAAAAATAGCTACTTTATTTTC
ATCAAGCTCATATTCTTTTACGATAATCATATCAGAAGCCAAAGGATGAATTTGACGATA
ATCGCCATAACATAATTTAGAAGCTGATTTTAGAATTTGTCCTTTGAAAAGTCTGAAATT
ACTAATCCAACGGCGAGTAAAAATATTCTCAGGGTCTTCTTTATTAAGATGATAAGAATT
AACATCACCGAACCAATTATATCCTACTACATCTTTGGTTCGTTTAATTTCTTTCCCAAA
ATTACCAAGAATATCCCGATTAACCAAATATGAAAGAAGACGAGACTCTTTAAAGGTTTT
CTTGATAACATCTTTATCTACACGGCTAGAAATTTTACGAATTTCATGAATAAATTTAGC
AGAAGAAACAATACTTGCATCAACACTATTGAGCCACTGATTGATAATAGAAAGAACGCT
ATTTTGGCCAAACATCGGTATAGCTTTATCATCAATAACTCTATTGAATGATTTAATATA
TTCGTTGCGAGTCATATTAATCTCCTCAGTAGAAAGTAAGAACATTATACCACATCCTTG
TGGCGAAGTAAACTAGTTTAGTGCATTTAGTGCGTTGTTCAATTTAGAACGTTGCTTCGT
GAGATTTTTTACTTTTTCTTGAGCTTTTTCTAGCATCTTTTCAGCTTCTAACGCTTCATT
AGTTGCTTTAACGAGTTCATCATCTACTGCCTTGATAGACTTCTCAATCGCATCCGCGTG
CCATTTTTCAACAGGTTTAAGACTAGGATTTTCAACAGGAAGAAAATTCACTTTATTAAA
TTTCCATGCATCTTTATTACCTGAACTATACATCCAAAATTTAGGATCGTTTGATGAGCA
ATTAGATAAATTTAAGTTATCTTGTACTTCCTGCTTTTTCTCTTGCGTGACTTCGTCTTT
TCTCAAAAAATCATATTTAAATGAAACGATCATATCAAGTTCATATGTTGTTGTATGTTC
CCAATTTCGCTCAAAACGAGGTAAAATCTTAGATTGAACAGCAGCAACAACATCCATATA
CTTAAATGCTTCTGTCAGCTGTGATTTAAGACATTCACAAATTGAACGAGAATTTTTAGT
ATTAGGTTTAAAGCTAATTCGTGCAGTTCTATTATTTTCTTTAGATGGTCTTACTTCCAT
TTGAAGAGTATAACCATCAAATTTCAGATTCTTTAAGTTAATATCAGAACCTTTTAATCG
ACTAGCAGTAGACAAAATTTGCTTTAATTGTTTACGGAATAGAGCAATAAATCTCCATTC
AAGACGATAATGATTTGGATTAAAAAATCCTGTTGATAAATCAACCTTACTCTGACCAAC
GCCTTGAACAAATAGAGGATTTTTATAATCAATAGTTTTACAGAAATCGGTGAGCTGCTC
ACGAGCAGTCATTTGTGTAATATATCCCGCTTTACTAAAATGACGTAACCATTCACTGCT
ATTCATATACTTAAAGTGAATGGAACTATTAACTCTATCAGGGTCTAAATTATTTTGGCA
CAAAAATGTCATAACATTACGGGTATAGCTGGCATTACGAACCATATCTTCAATTTGAGA
ACGAGTTTTCATGGTGTTCCTTAAGATTTAAGTAAATCAATAATTTTAATTAATTTTTCA
CGCTCAGATTTAGCTTTACTACTCAATCCAGATAGTCTAAAGATTTCATCGTCATATTGT
TGAATAGAAATATTTAGCTCTTCTATTTGTTTACTGAAATAATCAATCTGCTCGGAATGT
TTTTCGTTACTACGAACTGGAACGGGGTTTATAGGCAATTTAGTTAAACTGGATTCATCC
GGGCGATAAATTAAAATACAACTTGAACCAATCGGGCATTTAGCACCAGACACATATTCT
AATGTTCCGGCTTCTTTTAAAACTTCAAAAGCTAAACAGAGATGATGCCCCATATTAACA
TAATCTGTAGAGCGAGCTCTGACGTAATAATCATCGCCGCGAATACTAAATTTAAAGCAT
TTAAGGTCTTCTGTATTATTGGTCTTAAAAATCATTTGTTTATCTAGACCTTTAGCTAAT
CGAGCGCCTAATGCTAATAATCGTCGTTGATTTTCCCACAAATCTGCGATCATTTGGTCA
AATGATAATTTCGGCATTAGTCGACCGTAAAGATCATAGCCTTTATTATAATTGCGAAGA
ATTTCACTTGCATTAATACGAGACAATGCTCCAATTTTATTAAGGGCTTTCATGATGCGA
TTAGATAAACCCATTCCAGACCCGTTTGTTCGCTTCAAATGTTTTTCTAATCCAAATCCA
ATATCAACTTTAAACTTATCTAAAATTTCTGCATGAACATCTCTGTCAATAACATTCAAA
TCCAAAGTTGGGTTAAATCTATGAAAAAATTTATCTGGCTCTCCACGACGAAGTACAGTC
CATTCATTCAATTTTTTATTAACTAAAGATTTAATTACTGCATTGACATTATTAATTACT
ACTGACATATTTTCCTCACTCAATTTCAATTTTACTAAATACGCAGAATATGATGGAAAA
GACTATATAAGCACCGCATACAGATTGAACTAATACCATTCCAAAGAACCAAACAATATT
ATCAAACAATGTCTGTTTTACGTCGAAAGGACGTAAACTTACAGTAATATTATCGCCTTT
TTCTATTGATGAATACATCTCTGGTGAAATATATTCGCTAAATCTATAACCGTCTTTGAG
TTCATATACAGCAATAAACGATAAACTAGACCCCTTTCCTTGAGTTCCTGTAAGGGTATT
AACTACAGTAACATCATAATCTTTATAATGCATATAATCATTAATTGCGTAATAACCATA
TGCAACTACTACACATAAACAACATATCAATAAATTCAATCTTTTAATTATCAACTGTTT
CATAATAATCTCAATTAAAAGGGCTTAGAACCATTATACCATCCTTGGTATAAAGCGGTT
ATGCGAGTACCGTCTTTAACCGTTCTTCAAACTTCCGAAGAGTATTCTGGCGTTCAGCTC
TTTGCTTTTTGTAAGTTTCAATACGCTCTGAAATGAGAGTGTATCGTTCATTTACTGATT
CTTTCATAAAATCAGGAATTTCACGAGAAGCTTTAATCTCGTCAAATTTATCAATAACAG
CTTGCTCTTCAGCAATTAAGTTATCATACATCAAAATATCTTTCTTGATGAACTCAATAT
CTTCTTGAGTTACACGAGATAATTTAGATGCTTTATCCTTTTTGTACTGTTCGTTAGTAT
CACGAGACCAGTGTAATGTACAATTTTTATTCGTATTTTTGTAAATTTCTACAATACCAA
TCTCATCGATAATAACGATCCAATTCCAACGAGATTTGTAAATTTGTCCTCCATCAACCG
TGATTTCACCTCCGATTGAGATGTCATTAAAGAACTTGCTTTGTGCTTCTGATTTAAATT
TACCATCATTGTAATTTACCAGGTTGAAAATATCTTTAGCGTTCATTTTCTGTTCCTCCG
TAGTTGATAGTTGTATAGTACCACAGAGGAACGGTCTTGTAAACAACTAAAAGAAACTTC
TTTCACAATTTTTTCCACTGAACCACGCGCTCACTGCTTTCTTAGTCTCAGGAGCAGTGT
TATCCATAAACCATTCAAAGGCAGCCTTTTTATGATTCTGGAGGGCTTCTCGGGCTTTAA
TCTGCTCACGGTCTATTAACACTAACATATGAGCCTTTCTTGTCACCATGGGCTTCTTAT
GATTTTTTGAATACTCCCAGTCATTTGTCCATCGCATTGTCGTTGCGAATTGAAATACAG
CTTCTTTAATTTTAGTTTCGTAAATTTCACGAGCCTTTGAGTATAACATCATTACCTCCA
TTTACCAGTTTAATTCTAGTCATCTTTTTGATGGCAGTCCATATAATCTATTTCTGAACT
GCCTTTTTATCTTAGAAGTCCTCTTATGAATTATTTCAGAAGAGTAACCTCAGCAATTTC
TTCCCAACCGTTTTTGTCTGTCATAATAAAGTCAGCAAGATAAAGAGCAGTACGCAGTGA
AACATTGCGTAAACGATTAACATTGACTTTCATCCATGATAATGCTTTATAAGTTTCTTC
ATCAGAAAGACCGCGCTTTTGCATCATGTCAGTTGAAAGAATAACATCTTCAACCCTGAC
CATAATTTCTTCATTAGTGTGAACACCCAAATCCAAATAAACTGAGCGGGACACTAATGC
TTGTAAATGTGGAGCAAGTTTAGTACCACGGTCTAATTCGCGGTCAATGTCAACGTTTGT
GATAAAAACAATCGTTCCTTTAAATTCAAACTCACGCTCAATGCCTTTTTCTTCTAAGTA
AGAAGATGCGGTGCTCCAGCAGACTTTACGGGTCTCTCCAGTGTCCAGAGCAGCTTTCAG
AAGATTAAGAATGTCCATATCAGAGAAAACATCCACATCATCAATCAAAAGGACAGAATT
CTCTTCACGATTATTCCAAAGCTGCTCATAAAGACCAATACCGGAGATTTTACCATTAAT
GCTTTTATATTCAATGTATCCAATATCATTTGCTTTATTTAAAGCTTTATCTAAAGAATA
CGTTTTACCAATACCTGCTGCTCCTGAGATAATTAATGAACGAATATTTCCATTAATAAT
ACCATTCGTCATCATTCCCATAACATTAAATCTTTTATTAATGCGGGTTTTCATATCTTC
ATACGATTCTTTAACTTCTTCAACTTTTACACCATCATATGAAATATCTGATTTGTAAAC
CCAAACACCGCGACGTTTACCGTCAATTTCAACAAAAACTTTACCATCTCCTTGCGCATC
TACCGGAGCATTATCTGGGAACCATTCGCCTAAGAGCTCAAAAGTTCCAGAGATTTCTTT
ACCGAAGTAGATACCCTTATTGATAGTTACAGTTTTCATTTTATTCTCCAAATCCGTATC
AGTTGATAGTTGTATAGTACCATAAAGCTTTATGCTTGTAAACAGTTTTGTGAAAAATTT
TTGAAATAAAAAAGGGAGCCCGAAGGCTCCCTATCATTTATAATAACTTCGGTGGTTTTC
AAGATAGACCTTCTCAAGGAAGTCATCCCAGAAACTCATGTCTACTGTTTTAGGCATACC
ATTCTTAGAAGCTTCAGTAGATGCTGCTTCTACTTGATCGACCACATCTTCCAAAAACTC
TTGAACTGTTTTAAATGGATGTTTACCCAACTTCACGTCGAGAATAAATGGAGCATCTTG
GAGTGGATAAATCAAGTCGCCAGTTTTGTAAATTTCCAATAGTTGAAGTCCACCACGACA
AGCATGACTTAGTGCTTTCCAATCAATACCTTCATTAGCTTCGGCCTTACGAGCACGTTC
ACCATATTCAGCATCTAATTTGTTCAGTGACTGCTTAAGCTCAATAAGAGAAAGCGTTGT
CTGATATTTACGACCCAGCACTGTATAGAACGTTTGTGGACCTGTTTTCTCATGATTATG
GAACACCCATTCACAGAATTCGTTTTCCGGAAGACGATGCTTAATATCTTCAACTTTAGT
ACGACGCTGCTTAATGGAACCGTCTTCTTGGTAATCAACCCATTGCTCAGGGATTTGATT
AACTACTTTCAATACATCACGTAATGCAGCCAAACGAGAACCCTTGACGCCGTATTTAGA
AGCTTGCTTGCGGACATATCCTAAATAGGATTTCATGTTAGTCGTATAAAAACGAGAACG
GTTGTCTTGAATAAACTTCCACACATCAGGCAAATCAGATTTAACCACTAGTTCAGGTGG
AGTGTGAAGCATATCCAATGCTACAGTTTCACCATCTGCTGCTAATTTAAAGAAATATTT
AAGACTGTATAGTTCATGGTCAATATCATCTTTAGTGTTTTTAGATGATGTGTTGTTAGT
GTTTTTACTCATGTGCTCTTTGACATTTCCGACAAGAATATCGCGAGCAGGAGGAACAAA
GATTTCTTTAAAATCTACATCAGATTCTGGGGTAGAAGTTCCATAAAGATGACTACCAAA
ATAGCTTTTCATTACTGTTTTCATCATTCAGCCTTATATTCAATAACAGGACATACTTTA
GCTTTACGAGCTTTTAAAAATTCAACGATAATCGATTTCTTGGGTTGAATAGGAGGTAAA
CCTTTATTTTTCCTATCAATGTTTTCTACGTGTAAAGCATAATCCTTTTTCCATTTATAA
TCTTTATATTTTTCACGCAAATGTACAGGAATAACAAATGAACCAATAATAAGTAAAACA
ACCAAAAGCGCAAGTATAAATGCTGATGGAAGCAAAGTCCATAAACTACCAAAAATAAGC
CAACGCTGTAAACTTTCTGCACAAATAATTGTTAGCCCTACGAGTATAGCACACCCAAAA
ATAGAAACGAAAAATGTAGGGATAACAATAGACCAAAAATATGCGCATAGTGTCTTAGGT
CGTTGCCATTCATCGTTAAACAATTTGAATAATTTATAGTGCCAAGAGTTTTCATTAATA
ATCATAACTATCCTTTCATTGAAGGCGTAACAGTTGTTAAATACTTAATCATAGCTTCAG
CTTCTGATTTTGATAGTGAAATTTCTTCACCGTTTTGGCGAATAGAAACAAAATCTGGAT
GACGATCACCACCAGCTTTTAAAACACATATAAGTATCAGCTTCGCCTTCAATATTTGAA
ATGATTGAAGCAGTTCCACATGAATGCGGTTTTTGATAAATCAATACAGCTTTTCTTCCA
CCATTTGACTGTTCATAGGCTTTTAATACATCAAGCAAATCAGTTCTTTTATCAATAGCT
AAGATGTTATTTTCATCTTCAATATTAAATCCTTCGGATACTTCATAAAGAGTTACCCAT
AAATCACCGATGTCAATAACAGCATTTACTTTTCCACGGCTTTCAAGAGTATGACAAATT
TTCTTGATTTCGTCATAACTCATTTTAATAATATCATCTCCTTGACTAAGGAAGATGTCA
TTTTTAAATTCAACACGAATTAAACCACTTCCACTTGAAATAAACATATTTCCTCACTTT
GAAATCATAATTGGAATGACAGAATCAAGATAAGTCTTTAGCGCAATAGCTTCGTCTTTG
GTAAATGTGACAATATGTGCTCGAAAATCGTCAATTTGACGAATAGATACTACATCTCCC
TCTTCATAGCATTTTGACACATTTAAAATAGTTTCATCATTCTGATTGCAAGCGTTAGTA
ATAATAGCATTGCAATTTTTAAACCATTTTAAACTGTGTTTTCTTTCATTAGAATAATAA
AAATATTTAATCTTATCTATAAAACTATCCCAATGGATAACTGATAAGCATATTGACTCG
CTTTCAATATTAAATCCTGGGCATGAAGAATAAAAATGAATTTTATGCTCATCATTAATG
CTTACAATTTTATCAGTATAAGGATTTTCAATTTGGGTTAAATGAATGATTTCACTAGGC
GTAAAATATAGCATATCGTCTTCTTGTGTCAAACGATACATATTATTTACTTTTTCTATA
GCCAATTCACCAAAAAGTGAACTAACTTCTAATACTAATCGTTTCATATTAATCCTTTTT
ATTGGCACGAATTTGTTCTAAAATTTTAGAGTATTCCTTTGTATCAGATTTACGTACAAT
CGATACACTATACGGCATACTATCGGTGTCTTTTATAAAGCGTTCCAAATGACACTCTAA
TAAAATTTCTGGATTATAAATCGTGTACATCAAATTACCTGTTGCTCCACGATATTGACA
GATTTCTACTGATTGAACACCATTCCATAATTTTGTTAAGACCAAATCACCTACTTCTAA
TTTAGGAAAGCCCATGTTATTCACCAATTAAAGTTTCAATATAAGAATCAAGATTTACTT
TAGCTTCTTCCATAAGAGTTTTGTATTTAATCTTGTTAGCATGTCCGGAGAAAGCTGATT
CATCAATAACTGTTGATGTATGAACAGCTCTACTATACGTCTCAATTAATTCTCTTAATT
TAAAAGCCTGCTCACTTGTCATTACATACTCCTTTGAATCATATCAATAATATTGTTCAC
CAGATTATAGGTAAACATTGGGTAATTATATTGAATCATCACATATATAACAAACAAAAC
TTTCATTCTCTTCTCCTTGACAGTTGACAAGATTACTATACCATAACCTTGCCAACTTGT
AAACCGTTAAATGTTGTTTTCAATAAAATTCTGAAGCTTTGTGTGAGCATCAACCATGAT
TTTCATTTCTTCCTTTGCGAAAGCTGTACCTCTTTCCCGAGCAGAATACTCATAGTCAGA
AACTGCATTAGCATATTCTTCAATTAGCTTCATTAAAAACATCTGCTTTTCAGTTTTCAT
TATTCCACCTAATCATTTCAAGATATTGAACTAACTTAGCTTTAGATTTATCCAAATCCT
TTTTAGCTGCTTCTATACCGTCGTATGAATATCCTTCACAATGCTCAACTGCTAATTGAT
ATGAATCTATTTCAATATCACGTGCTAATTTAATGATTTTTTCAAACTGTTCGCGTGTTA
GCATAATTAAACTCTCGTATTATGATCGATAATTTCATCAAGAAACATATCTAACGCTTC
TAAAGCATTATCAACTTTAGTTTCAAAGTCTTTAATGCCTTTATTCGAAATACCCGATGA
ATACCAAGCAAAGTCAATCAGCTCTTCATCTGCTTTACGAATTGAAGCCACTAATTCCTT
GATTTTATCTGCTTGCTCTATACTAATCATTATTCCACCACATATGACAGAGAGAATATT
GCACACGCCATATGCGTTGCAGCTTCATCACACATATTATAACGTTTCTTCAAAACTTCA
ACAAGTTCTTCACTAGTAACTTCATCCATGTTGACGAAAAAATCACCATTAATGATGACG
TAGATATTTCCTTCTTGATTGAGTGCTTCAATTTTCATGATGTTCTCCTCTTTATCCGAT
AGTTGTATAGTACCACAGCTCAAACGGAAAGTAAACCGGTAAAATGAAAAAAGTCTCCCG
AAGGAGACTAATGTTATTCGAGGGAAAGAAGATACTTACTCTGGTAAAACATTCCAGTAA
TATCATCTATCGTGCTTTGGATGGCTGGAGGCATTTCTTTATAAATGCTGTTAGATTGGT
CTAGTATGCGATCAATCATTTTAATTGTGTCGGTAGGAAGTTTACTGGCATCTGGAATTG
AAGGTGTGTATTTTCTACCAGAATACCCCAAATATTGCTCACCAAATTTATCAATCAAAT
CTGGCAACTCGGAGAAAATAAAATCGTATGCTTTGTGTCTAGCATAACTTTTAGTTTCAA
AATGTGCAGAATGAAAATAAGCTTGTGCAGCCATTAATAAACCTAAGTATTCATCCGCCT
TTGAAGGTTTTCCACTTTGTGAAAAGTCGCTGAATTTCATTCAGTCTCCAATTTAATGTT
CATAATTCTAGCGTATGATTGTGCCATCTCCGCGCCTCGCTCTATACATTCAAAATCAGA
AGAGCACGGGTCATTTTTATAGGTCGTTCTCATAAAACTATAGAATTGTTCAGACGATTC
TACGCTTTTATTTTCAAAAAGCATATAAACGTGCCTAATACCAGATTCCATAAATTTATC
AAAATGAGGATCGACATTCGCTTCAATCGATGGAGATAAAACAAATGACAATCCTAGCAT
GGCAAATAGTGCCGTTGCTTTTAAGGCCATAAAGGCCTCCTATCATTTTTGTCCTGTATT
TACTTTATGCCGATGCACGGCCTTAACTTTATCAAGGTATTTTTCAAAATTTCGCAATCT
AGTATAGTCTGCCGGAGATTGGTTGAGTGATACTTCTCGACGCAAAGCTGAAATAATATT
TCCAACTTCCCTACGAATTTCATCTAATTGAAGCACAGTAAGATTGCGAAGTTGCTTTTC
AGTTAATTGTAGCATATATACCCCTTTAGTTAGATAAACCTATTTATAACTTTTGCACTA
ACCGAGCTTTTTAGTTAATTCATTCCAATGTTTTCTACACAAAGAAACATAAATTTCATC
ACCAATACAGATTTGATTACCTTCTTTAACTGGTGTTCCATCTTCCATTAATCGAGCTGT
CATAATCGCTTTTTTACCACAATGACAAACTGCTTTTAGTTCAATAAGTTTATCTGCAAT
CGCTAAAAGTTCTTTAGAACCTTCAAACAATTTTCCAGCGAAATCAGTCCTTAATCCATA
AGCCATAACAGGAACATTATATGTATCAACAATTCGACTTAATTGATGCACTTGTTCAGT
TTTTAAAAACTGAGCTTCATCTACAAATACGCAATGAATATCTTTTTGTGCTTCAGCCCA
TTTATAGAACTCGAAAATATCCATATCATCTGTAATAATATTCGCTTCCTGTTTAATTCC
AATGCGAGAAACAACTTCACAGACAGAATCGCGAGTATCAATAGCAGGCTTAAGAACTAA
TACACTCATTCCACGTTCTTTATAATTATGTGCAGCAGTCAAAAGAGAAGCAGATTTTCC
AGCATTCATTGCTGCATAAGTAAAAATTAAACTCGCCATATCACCTTCTTAAAGCGTATT
CACATAAGCTATTAATTCATTTTCTTTCTTATCAAACCGATCAGCAAATTCTTCCTGCTG
TTCGGGAGATAGCGGGCCGTATTCATCATAAAAAGTGTTTGCTTCTATATGCTCATCTAG
AAGTTCATGGATAAGCTCAAACAATTTATCTTTTTGTTCTTTACTCAGACTCATATATTA
ATTTCCAGGAGTTTTAAAATAATTCGCTTTTGCTTAAATATTTCCATAGTTAAATGACCA
GTATCTTTTTGACCGATTCCAATGGCAAAACCTTTATTAACAGCGAGTTCAATTAAATTA
TCAAGCTCACGTAACACTTCGTATTGTAACTCAAAACTATTCATTTTGGTTTACCTTCTT
TAACGAAATCAGGATAAGGATAAAATCCTGAATAAAATCCTTCACCTTTATGATTGATGC
ATCCTTTATTAGAACAATAGCACCAATAATCGAAATCACAAGCCATTTCATCATTACATA
AAGCAAAAACAACAGGCCATTTACACTTTTCACAATGGGCATTTTTAAGAATACTAGTTT
GGCTCATAACCATGTAACCTTTAAGCAATATTCTTCTACATGTTGTTTACGACCTTTCTT
ATCAATAAAGGTATATTCAACGAATGTTCCAATGTAGTCTTTATCTACATCATGTGGACT
ATTAATTGGACATTTAGTACGGCAAATGCGTTCCCATTGACGAATAATTTCCACTTTGTT
TTTAGGATCAAACGGATGTGGGTAATGTACTTTCATAATCCACCACTATTAAATCAATAT
CAGGAGTAAACATGTCGATTAATTTAGAAACCTTATCCCAATCGCCGCCAGCAATACCAC
AACCTATGCGTGGAATGTAAATTACTGGTTTAAACAAAAGGGTTTCTGCCTGTAGATTTA
ATTCTATCATGCAGTTTACTAAAGCACCATAATCAAGATTAGGACCTGGTTCGTATTGCG
TATACAGGTTATAGCATATACCAAAACCATTAGGTTTATGTTTAAAAACAATAGACATAT
CGCCTAATTTATCACGAGAACCGTACTCAGTAGTGGTTTTATCTATTTCTAAAATTTTGG
GATAGGCTCTTGCTAATTGACCAGCTACACCTGAACCCATAGTATGAAAACAATTGCACC
CGTGCGCAATAATATTACCTTGAAGAAATAGGGCGACAATATCGCCCTTGATATATTTTA
CAATCATCTAGTACTCAATCCTCGATTATAAGAATCTACCAAACGGTCAACCATTGAATG
ACAAGCGGCTTTATCTTTCTCCTCCGCAACTGAACATTCTAAGGTATTCCACTTTTTAGC
ATATCGTTTTAACAATGTATCATTTTTGTATCTGCTTGATTTATCTCTTTCTCCGTCTTT
ATATGCATATATTAATTTCTGCGCAAATTCAGCTTGGCATGCTTTATTTTTCCCGCAATA
ATCCGCTGCAGTACGGTTTACATACTCTCTAATTTCAGTATATGATGTATCTGCTAACGC
AGAAAATGAAATTAATCCTATACATAAAACCAAAATTTTAGTCATTTACTATTTCCAAAA
GTTTATTATTTTTAAGGTAATTAGCCTTTTCTAGGACTTCAGAAGCATATTTAGAACCTG
CTTTAACATTCCATCCCGAATTATAAGAGGATATTGCTTTTCTTATATCGCCCTTATGTA
TATTTAACCAATAAGAAAGTTCAATGTACGCCCAGGAAGCTGAATTGGACCGTTTATTCA
ACATTCTTTTTATTTCAGCATCGGTCATATTATAACCAAGTTCCTTAACTCTTGCTCGCA
TAGTAGGCAAATAATTTTGGAACATTCCGTAGGCGTGATGCTTTGGTTTAGATTTTAAAT
TAACTCCGCCAGAGCTTTCTTGCCATAAAATAGCAGCCATTATATGACCTAATCCGCTCT
TGTGGATATTTTTGTGTGTTTTATATTTTCCATCCTTAGAAAATTGTTCCCCGAATTGAT
ACGCGTAACGCATGTTATCGAGTTGGACATTACTGAAAGTATGCTCGGAGCTATGTGCCA
TCATTGAAATGGCCAATAGACCAGCGAGTAGTGCTTTTCTCATGCTTACCTCATTGAGTT
TTAATTACTGCTTTAGAAGCCTTTCCTGGTAAACGACGACTGTTGATAATTGCCATCCTG
CATTGAAGTGACGGGTTTTTGAACTTCTCATTAGGCTTACAAACCGTAAATCCAAGCCAA
AGATTTCCATCTGTGATTTCTAAACGTCCAGGACGATATTCAACCCCATCAATAAAATCC
TCGTCAATGTCAGGACGCGGAGGCATACTCAGGAATTCATTAACTTCTAAAACATGGTCT
TTTATTTTATGGAATAATTCAAAAACATATGTCTCATCAATCTCCCGTTGAATTGCACGA
TCAAGAAGATGTTGAGAATATTTTAGATGAAACGATGAGACTCCTGCTGCTTTTGATGCC
TCACGAATCTCATTGTTAATTTGACGAAACTCCGACTCAAAGTGACGACGAAGCTTATTT
CGACGGATAAAAACTTCTGTATTGATAGTCATGTTATTCTCCTCTTAACTGATAGGAAAA
TTATACCACAGTCAAGAGGAAAAGTAAACAGTTATTCTTTAAATCTAATCAATTTATTCA
TAGACTTTGAAACTTCTGCACGAACCTCATGCAAGTTTTTAAGCTGTTCAAGACGCTGTT
CATAGTAAGCAATTTCATCTTCTTCGAGACAGTCCTGTGAATCTTCTTTAAGATAGCGTG
CATAGTCCTGGAAAGCTTTACGGACTACTTCCTGGAAGTCATCAAGACTTTGAATTTTCT
TAGGAGCAACAGATACACGACGAGGGGCAGTATAATACTCATAACCAAACCCTGCGCTTA
ATTGAGCCATTAGTATTTTTCCTCTGGTTGGAACACTGCACGACAAGCCCACATACTGGC
TTCTTTGAGTTTCGTTTTAGCAATAGTTAACTGATCGAGGCTTTCGGCGTAATTCTTCGC
GAGTTCATCATCTTCACAGCTATCAAATGCTTCCCAAAATTCATCGTATAAAGCATCAAA
GATAAGTCCTAAACGAACTTCAGCGTCTTTAATAGCATTTACTTTACCGATTTTTTCGTC
GGTATGTGGCTTATAACCTTTAATATCTTCAATCATATTTGACTTCCTCACCAGTACATA
AATCGTATTCAACTAAACGAATAGGTTCATGAATGCCATATCCCTGAACAGAAATTTCTG
TAGTAGGATAAATTCCATCAATATCACCCATATTCCACGCTTCGTTAAATTGCTGTTCGC
CTGAATTACTAAACCACTCGGCGAAAGCATTTAGCACATCTTTAGAACCTTCAATAATTA
TCTTTGCCATTATAAGCTCTCAGTAAAGGTACGAGCAATAACGTCGCGCTGCTGTTCCGG
AGTCAGAGAGTTAAAGCGAACTGCATAACCGGATACACGGATGGTCAGCTGCGGATATTT
TTCCGGATGCTTAACTGCATCTTCCAGAGTTTCACGACGCAGAACGTTAACGTTCAGGTG
TTGACCACCTTCAATTTTAACTGTAGGCTGTGGCTCAATTTCAATTTCACGAGCATGCAA
ACCGTAGAAAATTTCTGGGTCTACAAAAGAGTCCTCTTTAAAGGTTTTAGAGACAATAAT
TCGTGCTTGAATACCATCTTCAAAATAAATAGTACCTTTATGTGTGCCTTCAAGAATTTG
ATATGCTTTCATATAAACCTCAATTAGAAAATAAATTTATCCAAGATTGTTCTTTAATTA
AAAATGGCTCAGAATCATATGCCATTAAACTTTGGGTGATTAGTCCTTTAAAAGGTCCAT
CAATAAATTCCATGGTAAAATATGGAATTTTATTCATTAGCCGTGCATTAGGAGCAGTGC
ACAAAACTCTGCATCCTTTGAATACACCTTTTTGTAATTTGTATTGCTTGGGATAAAATT
CGCTCAAAATGTTATTATTTGCTAAAATTTCAAAATGATTCACCAATTTATTTTTAATAG
TTTTTGGCGAAAAATAAAGATATTCGAAAAGCTGAGTGTCTGTCATCATTGCATTCCGAT
TACGAAAAACTGTGGACGAGTAATACCACCAATGCAACATTTACTATTACAGCAGTAGTG
TACAGTGTCAATATGGACACTATAAATCTTATCCATATCAGGAGATTTGACAGGCTCGTC
AATTATATACAAAATTCGCGAAAGCGATAAACCTCTGAACTTGCTTCCTTTATTACCAAT
AAAACTGCGCACAGAATCAGTAAATAAACGAAAACGTATATCATCATTAGAATAACGCGA
AAATTCCTTTTTAATGTTATTTGCAGAAATTTTAGCGTAAGCTGAAGTGTTAGAAAGAAC
AATAACTGTTCCGCCATCATACAGCCAATTAGCAGCAAAGTTAGTCACAGCAATTGATTT
ACCAGATTGACGTCCACCATCTATACGAAGTGTACAATATTCTTTAAATAAATGCGACGA
ATCAGGTACATAAAATCCACTATTACAAATTTCTTCTACTCTAGCATCAGAATGGTGTGT
AAAAGCATTCATCAGGGATAGATAAGGACCAGTTAAAAATGTTCTCATTTCTTCTCTCTA
AGTTTGGGCCATTCCGTGGCGCATGAATTGTCCATTTCTGTATTTACCCATTACCGCACT
CAGGCTCGACCTTATTACAGGTTGGCGGGAATCCCTCATAGAATCATGAGGTCCAGGTTG
TTCCCATATTATTTGAACGATTTAACGTACTGAATAGAACCTTTACTATTCACAAAGGCA
ATTTTACCATTTTTATTATACATGGTAGCAGTACGTTGATAGCCATAAGTTTGTTTACCA
GATTGATAACCAACTAATGCGAAATTATCTTCACCCATTTTATTCTGTTTAGCGCGAATG
CGTTCATGACCATGCGCCATAAGAACCTTAGCATCACCAGTTGCAATGGATCGACGAGTT
TTTTCATCTTCAACTGCATTAACAAAGTCGTCAAATGAATCAAATTTAGCATGAGATTTA
TTATACAGTTTTCCATCTACTGCATAATTATATCCCTGTACATCCATCTCAACTTCTGTA
CCATCATCCAATACAGCAGTGATTTTTTGACCTTTTTTAAATTTCAAAAGATCAGTAAAA
GATTCAGCAATAATTTGAGAATTAATAGCTGCTTCAGTGATAAATTCTTGATATGTTTTC
ATATGTTTCCTTTAAATGTAAATATTTTTACCGTAATACTTATACCAATGTGGTTTCATT
AAAATTTTTTCATCAAGTCGTTGTTGACTCAATTTAATAGCTGATTTGCATGGATTATAA
TCATTTTTCCATTCTACTGGAATATCGCTGATGTCAGGAACTTCAGTATCTTTTATACTG
AACCCGCGTTTTAAGCATTCCGTTATAATGTCCGATTGACGCTTTCGCAAAAATTCTAAC
TTATCGTAAAAGAAAGTAACATGACCAGAACCTAAAATAAATTTAGAAGATATTTTAAAA
TCTTTAACGCGCTTACCGTTTGCCACATGCTTACGAACTATACCAAAAACACGCGGCAAT
TCACGGTATTCTGCGATTAAATGTTGATCAGCAAGTTCAGATACTAAAGTCAAATTAATA
CGAGTCATTTTATCCCTCCAAGTAACTGTGAATATACTATCACAATTCTAGGAGAAAGTA
AACAACTTTATAGATTTTTATACGCGTCCCAAGTGCCAGTTCTAAACGTTGCAATGACTC
GTTTTGCGCGATTAGGTGTTTGATTATACCATCTACTTTTAGCTAAGTTAACTGCTGCTT
CATCCCAGCGTTTTTGTTGAAGCATACGCAAAGAGTTAGTGAATCCAGCTACGCCAGTTT
CTCCCATTTGGAAAACCATATTAATCAATGCACAGCGACGAACAGCATCAAGAGAATCAT
AAACTGGTTTTAATTTAGCATTTCTCAGAATTCCACGAACAGCAGTATCAACATCCTGAT
TAAAGAGTTTTTCAGCCTCATCTTTTGTAATTACACCATTGCAATTACGTCCAATAGCTT
TATCTAATTCAGATTTGGCAACACTTAGTGATGGACTTTTAGTAAGCAAATGACCAATGC
CAATAGTGTAATAGCCTTCTGTGTCTTTATAGATTTTAAGTCTAAGACCTTCATCTATAC
GTAACATTTCAAATATATTCATAATACCTCCTAAGTATTTATAGAAGGTATTTATAAAAT
TAAAAGAGGTTGTTCATTATTCGGTAAAGTGAAGGACCCATCACATATTGCCACTGAGTA
CGAGGAATAAGAGCAAAAGCATCCATCTCTGGAATCATAACACCATCTTTATTTTCAAAA
TAAGACTCACAATAGCAATTTCTGAACATCTCATGCTCTACTGGAATCGTATAATAAAAT
AACTGTAAGTCTTTATTACTAGAATATTTAAATACACCTAGGTCTTCTAGAAGGTCTGGA
TTATAATTGCTAAAACCGGTCTCTTCTAAGCATTCTCTTTTTGCAGCTTCCAGTGCATTC
AAATCAGAACTTTCTACACGCCCCTTTGGAATATCCCAGCGATGTGCCATCATTCCAGGC
TTACGAGAACCAGTAACCCGACCCATAAATAAATCTCTATCTTCTGTCATAAAGATAATA
CCAGCTGATAGTGTTTTCATTTTAATTTCCTGCATTCAGTGATAAAGTTATTTAAATTTT
GAGCGTATTTCTTTTCATCAAAAATCTTTTGCTGTCTGCGTAAACGCCATGGCATTTCAA
TGAACGTGTACCATATCCCAGATAATATTGCTGCTGTAAAAATATTAACAAGTATGGTTA
AAAGAATCCAATCTCCTATTCTGTCCATTGGATTTTTTATAAAAAAGTAAAATACGAATG
ATGTCACAGGAAGACTAATAATATACCACAGAATCATAATTTTATCTGTGAACCATTCAG
CGTTCGTTAACTTAGCGCGACCATTATGAATACACACGAATTTATCATCTGTTATAATAG
ATGGCTTAGCTGCTTGATATCCCATTCTAAACTCCCTAATCAACAAATTTTTCATATCTT
CGGAACAACCATTCCAGTCAACTCTATCAACTGAAATGCCATCATCCCCATCATCTAAAT
CATACCAGCGAGTTTTTAAAATCATTTAATTTTCCTACAATCACTCACAAACTCTTCCAT
TGATTCTTTTTCAATATAAGACATGTAGCTATTATATTCCTTTAATTGTATTTTGTAATC
CTTTTTTCTTTGCCAATTTATTTTAAAATTATCATAATGAAAATATAACATTGTACCAAA
GAATGAAAATAATGAAATCGCTATAGTAAAACGAAGTTCACTCCAAACCTCTGTTATAAT
TACTGTGCCGTCGATGTTTAAAATAAAACAGTCAATTAACAATCCAATAAGACTACCTGC
AAGAGCTGCAAATGCTGATACGGCAAGAATTAATAGTGCCTCAGGAAACGAATATTTAAC
TTTATTTAGTTTTGGCTTTTGCATCGTGATTCCTTAACAAATTTCATAATTTCATCAAAT
TCATACATATCAAGTTTAAGCTGATGTTCTTTTTTAATCTTTTTGCACTGGGCTTTCCAA
TCACGTACACGTTTACGATAGTGTCTTCCTTGATACCAGTATCCTATCCAATTTATAGGT
ACTAATAAAATTGGAACTACAAATGGAAGAATTAGCGTTGACGCAAATATTGTATTAGAT
TCAATATCAGTCATAACGTCTATAACCATTCCAGCAATCAGCAGAATCACAAATGATACG
GCTACCACAGGACCTATTAACACATCAGTAGAAATTATCTGACGCTTTAATTCATACTTC
AACGGTTTACTTGGAAGGTATAGTGATGGCTTTGACATATTCTCTACATTCCTTAACAAA
TTTTTCTAGTAATAAATCGCTTTCAAAATTGGGATTTTCCACAAATTTATCAAAAAGATC
ATCAACAATAGTTAAGATATTCTTTTTACTAAGAATACGTTTATTTTCATGCTTCGTTTC
AGAATCAACTATAAGAGTAAAGAAATATTTCTTGCCCTGAAATTTTACAGTAGTATCAAT
ATAAAATAAATTTGACTTTTGTAAATTACGTTTAAACCATGCATCACTTAAACTATAAAC
GCCAAGATAATCATAATCGTCGTTTAAATAACAGACCGTCCATTCAGGAGAAACAAAATC
AGTAAATTCAACGTCAAAATCGCATGTTAATAAATGAATTGATTCAATACTGTTAATAAG
TATTCCAGGACGTATTAAAGACTTTTTACCTCTAGAAAATCCTCTAGAAAGACTTTCATC
AGTTTCATATGAAGAACCCCAATAATAATTACGTCCTTCTGCCATACGTTTAAGAGCATT
TAATAATTGATCTGGAACATCAACCTGTCTTTGGAACTCTTCAAACATTGAATTGAAATC
ACTTTGCATTTTCATTCCTACTTACTCCAAGTAATAGGGGCCGAAGCCCCTTATCATTAT
TTCAGAGAATTAATATATTCCTGAACATCGGCAGAGGTAGTTTCAACCCCAGAAATATTA
CCATTAAAGGTTTCAACTCGGGCTAGAGTATCTTCAATATCAACTTTAGTTAGCGCTGCA
ATTTCAACTACATCATCAGCAGTACTAATTCCAAGGGCATTTGCTGCACGAGTTTCACGG
ATATATTCCAATTTAACTGCAAGTTCTTGGCGAGCATCATCCAATTCAACTACTTTCTTG
GCGATTTCAATTCGCATTTCAGCATAACCATCAGCTTTAGTAGTCAACTGTTCAGCTGTT
CGACGATATAGCAAGCCGAGTTTAGCATGCATTGTTACATCTTGACCTTCAGAAAGAAGC
TTGCGAATTTCACGCTCTTTTGATTCGGCCTGTCGATTCTTTTCGACAATAAGTTCACGA
ATACGTTTTTCTTCATTAATAGATTTAACAGAAGCAGTTTTTAGATCTTTAATCTTATCA
AGTAGTTTTGCTGCCGCAGCAGTATACTGTTCTTCAACAGATAGATTTTTAGCCATAGCA
GAACCAAGTTTAGTGCGAATAAACTCAACAATTTTCTTCAGTGTGTTCATAGTATTTCCT
TAGGTTGGTATAATTAGATAATATCACGTTTCTAATAGATTGTAAACTTATTCTTCGTCT
AGCTCGTCGATAAAGGCGTCGATGGCCTCGACAACGGCATCATTGATAGCCAATAAAATA
AAATCATCGTCAGTACCTTTAGAAGATTTTAAAGCATTGATATATGCTTGGTTGGCGAGT
TCCCAAGCCTTTTTAAAATAAGGAGTTTCATCATCGGGGCCAACGAAACGTACGCCTTCA
AAGATACGGTTAGCATAATCCAATACCCACTGTACAGGCATGTTTTGAGAACGTTCGTTA
AACTCTTTAAATTCCTTAGATTCAAAAAGCTCTTCAGGATAATCATTTCTATTACAAAAA
GCTTTACTAAAGTTACGTTTCATAATGTTTTCCTTGTGATGATAAGTCTATAGTAACGCC
TTCATACTTAGATGTAAACTATTTCAACATCGCCTTTTGCTGTTTTATAACCTTCAGCCC
AAGATACAACCGAGTTTTTATCTGAAGATTCAAATTCTGGCATTGGGCTTTCGCCAATGT
TCACGCAGTATGTTTCTTCATTTTCGTCGAACCAAATAATTAATGTAGTCATTTATTTCT
CCGTTTCTGTATTTGTTTTGATAAGTCTATAATAACACCGTCCTTGGTGTTTGTACACAA
TTATTTCATATTATTGAAATATTCTTCTGCGATTTCATCGTTATCATGGTAAACTTTAGA
AGACAGTTTAACATAACCTTCAGCAGTGAACATGTTAATCACAACCTTTACAGTATACCA
CTGACCATCTTCATTACCCATTACTGCGTAAGTTTCAAACATCGGATGGTCAGGACCGAT
AACTTTAATATCATTCACTGTACGACCGAAATCTTCTGAAACACATTTCATAAAGAAGTT
GAACAGCTCACCGTAATTATCCATTTCATTCTCCAAGTTGTTTTCTGTATCAGTAGTTGA
TATTGATATAGTACCATGGAAGGACAAGGATGTAAACCATTTTGTGAAAAAATTTTTAAA
AAGTTTTGGGGAATTCTAGGGCAGGAAGGGGAAATCAAAGGATAGGATAATATATTATAA
AGGGTAGAAACTAAATGATGTCTAGAGAGGCCTGGAAAGGCCTAGATACCAAAAAGCCCC
AACCTTTCGGTCGGGGCTAAGAATGTTATTTGATTTGTTTAGCAGACCAAATGCGGTCTT
TAATAATTTTTTGGATGTCTTCAACATACTCAAGAGAGTGAACATGTGGGTTATCTTTGA
AACTATAAGCACGAGCTAGCTTTTGGCCTTCAGTTTTGATTACTAAAAGCTCTTTAAGGA
TAGCTTCATATTTGGAGATAATTCCTTTAACGATATTTTTCTCTTGTGTGACTTTAGGAT
CTGCTTTAGGAGCAGGTTTACCTGAAGCCTTTGCAAAAGCAGCGCCAGTAGCAACTAAAC
TTTTCCAGGCCATACTAACAGCATTCCCCGTAAATCCTTCTGCTTTCATGTCGCGAGCAA
ATTGGAATCGTGATTCATCAGAAGCATCTTTATAGGAATATTTACCCGCTGCAATGGCTG
CTTTTGCGACAGCTTGAATTTCGGTGCTAGACGCTTCATTTAACACCGCTTCATTTAAAA
ATTGAGTATATGATTTCATCTTATTTCCTGTTTTAATTTGTAGATTTAATATACTTATTT
ATACTAAAAAGCCCCAACCTTTCGGCTGGGGCTAAGCCTTGCGGCAACCTTGTCGGGGTT
CCACCTGCTAAGGCAAGTGTTTGTACGAAACGCCGGGATTCGAACCCGGTTATCAAGCAG
TTGACGCTACTCAATATTTTTAAAAGGCCATATCTCGACCATATCCGAACGTTCCGTCAA
AAACGCTACTCGGCTTACGGCAAAGATATTTCCTCGAATCGATAATTCGGTGCGCCGTTT
CTGCTGTGATGTAAGAGGGCATCAATAAACGCAAGGATTATTAACGCAATTCCTTACTCA
GGGAACATCAGTCCGACGACTTGCCGGTAGCGACCCGGCTTCTTGTCTGGTCGAGGCAGT
AGGGATCGAACCTACGACCCAGGACTTAGAAGGTCCTTGCTCTTCCTTCTGAGCTATGCC
TCGTATTTGGCGGACGTGATAGGATTCGAACCTATAACCAATCGCTTAACAGGCGATCGC
TCTGCCATTGAGCTACACATCCAAATTGGTGGGGAGTGGTGGAGTCGAACCACCCGAGTC
GCAATGACAATGAATTTACAGTCCACACCGCTACCTCTACGGGATAACTCCCCTAATTCA
TAATTTGGTGGCCCTGGGTGGAATTGAACCACCATCTGGCGATTATGAGTCGCTTGCTTG
AACCTTCCAGCTACAGGGCCTTGGTGCTGATTGACGGAATCGAACCGCCGACCTTCTCAT
TACAAGTGAGTTGCTCTACCTACTGAGCTAAATCAGCAAAACTGGCGGAGGCGATAGGAT
TTGAACCTATGAGTCGCCGGAGCGACTGCCGGTTTTCAAGACCGGTGCATTAAACCACTC
TGCCACGCCTCCAGTCTCCATACAAGGATTTGAACCTTGGACCTCCTGATCCCAAATCAG
GCGCTCTACCAAACTGAGCTACACGGAGTAAATTAAATTGGAGCGGATAATGAGAATCGA
ACTCACATCATCAGATTGGAAGTCTGAGGTAATACCATTATACGATATCCGCAAATTTGG
TGCGAGAAGTGGGACTCGAACCCACAAGGAAATCATTCCGCAGCATTTTAAGTGCTGTGC
CTTTACCAATTTGACCATTCTCGCGCTGGGAATAAAGGACTCGAACCTTTGCATCCTGGA
ATCAAAATCCAGTGCCTTACCAACTTGGCTAATTCCCAATTATTAACAAAGGCTCTTTAG
CAAGAACCCTTGATGATAGAGGGTATTAATCAGTGCGGTATGAGTTAATAATAACAAATA
ATTCTTAAAGCATATTTACCATTTATGATAATACATATTTACGATGCATTCAAGACCCAA
AGGATTCTTGAAAATATCATATTCAAGAGGACCTTTTTCTGTTTCAATAAAGAAATCAAA
ATTTACTGTATTAAATTTACGGTCTTCCTTCACTAATTTAACTTGAGAAGATGAACGGTC
AACATAAACCTTTTCAACTTCAAAACATGTTAAAATGCCATAATCATCAATCAAGGCTTT
AGCTGCTTCTTGATCATATTTATATCCATTTTCAATGGATGATACTTTAGCATAAAGAAT
CATTATCAACCTTCATCAACAATAGTATGAGTATGGGCATTTACAATTTGCCACCAATCA
AAGCGATTAGAACCATACATCGGTTTGTTTTCATTTTCTTTGATAATATCACGAAGTTCA
TCTTCAGTAGCGGCTTTCGCGATTAAATCAGCATATCCACCACGAGGATAATAATTATCA
CCTGCAAAAAGTAAAAAGTTTGGTTTAATGTTATTGAACTCTTCAAGAGTAATAACTGTT
CCTGATGGTAGTTGAACAATCTCTACATATTTATATCCTAATTTTTGAGCAGCATCTACT
GCTTCAAGCAAAGTATCATGAGCCCATCCAATTTGACAACCTGGCTTTTCAGATGGATTA
GCCTGATAATCCGCGTACACTTTATACATTCTATTCACCTTTAGAAATTTTATCCATAAC
GACAGCAATTAAACCAATTAAAAATGCTACTACAAGTGAAAAAACATTTTCTGCTGTAGT
TAATAATCCGCATATAAATCCAACAAACATTGAAAAACTAAAAGCAGAAGCAGAAATTGC
AATAGCAACATTTCGAATTAATTCACAACGTTTCATTTTATTCTCCTCAGTAGTTGATAG
GGTAATAGTATCACAACTAAAACCCTATGTAAACAACTTTGTGAAAAATTTATTACAAAA
GATTTTTAGCAATAATTTTAAGCTGAGCTACTGAAATATGCTTGGCTTTAAACAAAGCAG
TTTCTTCAGCAGGAGAGATAACGATTGTAGCACCATCTTTCTTAGCGGACCATCCATCAC
CCAGGTAAACAGTACCTTTGACTTCTTCGCCATCAACCAGGCTAATCATCGGTTTACCTT
CACGTCCTTTGTTTGCTTTAATAACTTCAGAAGTAAGGGTCGCTTCAGTCAAAGTAGAAG
CGTTAGTCGTTGAAGTGAATTCTTGATAAGTTTTCATTTTATTTTCCTAAATTAATAAAT
GTGTTGATGGAGATAATATCACTTCTCCATCATCGTGTAAACAACTTTGTGAAATTATTT
TAAATCGTCTGCCCAATCGAGTTTAAGAGGTTCTTTGTATTCACGATCTAATACAACCGG
AATTTGTACATCACCGCTAAATGATAAGGGCCCAACATTATAAGACAATGTTATATGCGG
TGTGTAATCATCAAAATCATGTGTAGCACCTAGTGCCCGCGCATACATGTGTCTACAGCG
CAGATATTCAGAATCTAGCACAAGTACAAGAGTCGATCCATCTTGTGTTTTCCATACTTC
TAAATGTCCAGAAGAAGCTACTTCAAAACTTCCGCTTGATGGAACATACGGAACATTTAC
TCTTGAATAACATATGGTCGAATGAATTTTTTCTCTAGGAACTGGATTAGGAACACGTAA
AGAGCGCTGAAGTTCTTCCAGCGCATCAAGTGTTAATTCTGAAAACTTAGCTGCTACATA
AAGACCCGTTGAAAAGTCTTTAAATTCCATTATTCTTCATCTTTTGCTTCATCAGTAGAT
TCAGCAGTAAGATTCTTAACAGCTTCAACGATTTCTTCAACTTTGATAGTATCGCCAGTG
ATACCTACTGCACGAGCAATTTCAGCCAAAGTTCCTTGCAGAATTTTGGATTCTTCCATC
AGACGAGCCGCCTGGTCCTGCGTATCAAGAATGCGAGACTTCAGAGTTACGATTTCAGCA
GACAGTTTTTGTTCAATAGTTTGTTCAGACATTATAGTACCTTTAGTGTATTTTTAATTT
TAGAAAAAAGTTCTTCAAGAGAACCATCGTTTGTAATTACTAAATCGCCATCACGAATTG
GCAATCCAGCTTCTGTAATATGAGTATCATTGGATTTTTGACCAGGACGAACTACATGAA
TTACTGTAGCACCCATCGCCCTAGCCGCATCCATTTCATGATCTTGACGAGTATCAGGAA
CGATATAATAATCATAACCTGAGTTAAATTTATCAAGATAATCTAAAGCAAATAATTTTA
CCCAATACATGCGGTCGAAGTTATTAACAATCAAATCCGTACCTAGGGCTTGCATCAGAC
GACGGACTGACCATTGATCTTCAATATTATTTATAACGTCAATAATCTTGTTAAATGCTA
CGAAATTAACTGATTCTTGTCCTTCGTCATCAAAAACAAACACGCCTTTAATTGGGCTTT
TACTATTAAGATAGCAAAATGCTTGTTCCATAATCGTGATTACTTCTGATTTAGTCAGAT
TTAAATTAGTCTCACGATCATAGTCAATTCCTTCAAACTCTTTACGAGTTAAGCAAGGAT
AGTCAGTATTTGCTGCAAATACACCCCATGCATAAGCCAATGCATCTTTAATAGGACCAG
CAAGTTGATATTTAACTGCAGAATAATTATTCATGATAAAATCTGCAGTAGTATCTTTTC
CACTACGCTTTACACCACTTAAAAAGATTAGTTTCATATGTTTCTCCTCAAATTTAGTTA
AGATTATAACACATGAAACTGAAGCATTAAACTTCTGCTATAATTTTGCCATCTTTTTCT
ACTTGAAAATACGTATAAGGAATCGTAGCTGTACATACTAAAGCCGGGTCTGAATCTTCC
GTGTAGCTAAATTCTACTTCAGACAGATCAGAAACCCAAGGCTTATAAAAATTTATTGAC
ATCACGATTTCAGTTTTGCTATTATCTAAGATGTAAAGCGTAATGTACTCAGGACCTGTT
TTTTGGGCAGTATTTTCACCTGTAAGATAGTTACTAGTTCCTAGCATCCATTCATACATT
CCTATCCACGACTTAAGTTCTTCATCAACTATAAATCTCACAATGAGTGGATCATACTCA
AATGTAACACCAGGTCGTTGTGCTCGACCCAGGCCAAACGGCCCAGTCACGGTATCAGTA
ACAGGTATTCTAATTCCTGGAATAGGAACTGACTGAGCATTTAAAGTAAAAGCAGATGTA
GTATTACTATGTGGTATTGATACTACAAAGTTAGTTGTATTTGCTTGGTTAAAAATTTGT
TGCAGAGCTTGCGACATATATTCCTCATAATGCTTTATAACTGTTGGTGGTATAATGGGT
CTAAGTCCCTTCCATTCAATTCCATTTAGAACAAACAACAGAAAAGAATGGAAGATAATA
GAATTAAATATTTGACCAGACTTTGTTTGCAGAGAAACGTTTTCCTTTTGAAACGAACTG
CTGAAGTGGCATCAACACAACGTTCGCCCAGTCTTTCGGGGCGATTTCAACAAGGCTACC
CATAATATTACCAGGTATATATGCCTTAATCATTTGGTCTGCACCTCTAAATCCTTTCAC
TTGACTCCAATCAATTTTTAATTTCGTTTTATTAGTAATAGTAGGTGTATTTGCATATTG
CTTTAAAAGCTCTTCTAGGAATTGCTGACGAGCTTTAGGTGGAATATAGTGCAAGTTCAA
TCCGTACATTAAATTATGCTTACCTAAACCAAGGTAAATTATCAAAGGAAATTTATCCCA
GTAAGGAAGAGTTTCCTTGTGTTTAGCATCATAAGCAAAAGCATATATTCGTCCCGGCTG
CGGGCGAACAACTTTATGTCCTTTTACTTGCTTAATAGTTTCAGCAAACCACTTTCTAGT
TTTATTATTAATTGCTGCACCTTCATTACGAATTTTATCACGCAATGTTTGTCTGAATGA
ATTTATCATAAGCAGTTGTCTTTCTTGCTTATTGAGTTTATTCATTGGTTTTGATTCAAG
CTTTTGAATCTTTTCAGCCGTTTTAATTCCTGAAGCATATTTTGACATTGCCGAAGTAAA
CGTAGAGTATTTGATTCCTCTTTCTTCAGCAAATTGCTTTCCTGTCATTCCTTTTGCTTT
GGCCTTTTTATATTCAAGACCTATCTGAATCCATTTCTTTTCGTTTAATGATTGCTTAAC
CTTTGGAACTTGGGGAGTGCTTTCATTAATTATTTGAAAAATAGCCATTATGCCCCCTTA
AAGCCAAGAGCTCGTAATCCATCTTCTGTTAGAATTCTAAATTTTATTCCACGCTTTTCA
GCTAAAGCTTGCGCTGCTTTCCATTTGTCAGTATTAACTGAATATGTATAAATTTCATTC
ATAAATCTTTTCTTCGCTGCAGTTGTTAGATGTGCTGGTTTAACTGGTGGTTGTGTTTCT
TTTTTAGGCTTTATTTCAATAAAAAATTCTTGTCCAGAAGAATCTTTCATCCAAATATCC
ATGAAGTATCTACGTTTTTTCCCTTCTGCATTACAAAAATAAGGAATTACTGCTGTTTCA
CTACCCCATGCAATAATTTCTGGATTTTTATCTAACCATTCAAAAAAGAATTTTTCCCAA
TTTGATCTATACGTAATTTTTTTAGGGTCACCTCTATATTTTGATATATTTTTAGGAACC
CATTTTCCAGAATATGCCATTGGATTCTCCTTATAAATAGATAATATATTTATAAACAGG
AGGGCCCATGCTCTTTACATTTTTTGATCCGATTGAATATGCGGCCAAAACGGTGAATAA
AAACGCGCCGACTATTCCTATGACAGATATTTTTAGAAACTATAAAGACTATTTCAAACG
TGCTCTTGCGGGATACCGCTTACGTACTTATTATATTAAAGGTTCACCACGCCCGGAAGA
ATTAGCAAATGCTATATATGGAAATCCGCAGTTGTATTGGGTTTTATTGATGTGTAATGA
TAATTATGATCCGTATTATGGATGGATTACTTCGCAAGAAGCTGCTTATCAAGCATCTAT
ACAAAAATACAAAAACGTAGGTGGAGACCAAATAGTATATCACGTGAACGAGAACGGTGA
AAAATTTTATAATTTAGTATCATATGATGATAATCCATATGTTTGGTATGACAAAGGCGA
TAAAGCTAGAAAATATCCTCAATATGAAGGAGCACTTGCTGCGGTCGATACGTATGAAGC
TGCTGTTCTTGAAAATGAAAAACTTCGTCAAATAAAAATAATAGCAAAATCAGACATCAA
TTCATTTATGAACGACCTTATACGTATAATGGAGAAATCTTATGGAAATGATAAGTAATA
ACCTCAATTGGTTTGTTGGTGTTGTTGAAGATAGAATGGACCCATTAAAATTAGGTCGTG
TTCGTGTTCGTGTAGTTGGTCTGCATCCACCTCAAAGAGCACAAGGCGACGTAATGGGTA
TTCCAACTGATAAATTACCATGGATGTCAGTTATTCAACCTATAACTTCTGCAGCAATGT
CTGGAATTGGAGGCTCCGTTACTGGTCCGGTAGAAGGAACTAGAGTTTATGGTCATTTTT
TAGACAAATGGAAAACTAACGGAATTGTTCTTGGCACATACGGCGGAATAGTTCGCGAAA
AACCGAATAGACTTGAAGGATTTTCTGACCCGACTGGACAGTATCCTAGACGTCTAGGAA
ATGATACTAACGTATTAAACCAAGGCGGAGAAGTAGGATATGATTCGTCTTCTAATATTA
TCCAAGATAGCAATCTAGACACTGCGATAAATCCTGATGATAGACCATTATCAGAAATTC
CAACTGATGATAATCCAAATATGTCAATGGCTGAAATGCTTCGCCGTGATGAAGGATTAA
GACTAAAGGTTTATTGGGATACTGAAGGATATCCGACAATTGGTATTGGTCATCTTATCA
TGAAGCAGCCAGTTCGTGATATGACTCAAATTAATAAAGTTTTATCAAAACAAGTTGGTC
GTGAAATTACCGGAAATCCAGGTTCTATTACAATGGAAGAAGCGGTAGCTTTATTTGAAC
GTGATTTGGCTGATATGCAACGTGACATTAAATCACATTCTAAAGTTGGCCCAGTTTGGC
AAGCAGTTAATCGTTCTCGTCAAATGGCATTAGAAAATATGGCATTTCAGATGGGCGTTG
GCGGTGTAGCTAAATTTAACACAATGTTAACTGCTATGTTAGCCGGAGATTGGGAAAAGG
CATATAAAGCTGGTCGTGATTCATTGTGGTATCAACAAACAAAAGGCCGTGCATCCCGTG
TTACCATGATTATTCTTACAGGGAATTTGGAATCATACGGTATTGAAGTGAAAACACCGG
CTAGGTCTTTGTCAGCAATGGCTGCTACTGTGGCAAAGTCTTCTGATCCAGCTGACCCTC
CTATTCCGAATGATTCGAGAATTTTATTTAAAGAACCTGTTTCTTCATATAAAGGTGAAT
ATCCGTATGTTCACACAATGGAAACTGAAAGCGGTCATATTCAGGAATTTGATGATACCC
CTGGGCAAGAACGATACAGATTAGTTCATCCGACTGGAACATATGAGGAAGTATCACCGT
CAGGAAGAAGAACAAGAAAAACTGTTGATAATTTGTATGATATAACAAATGCCGATGGTA
ATTTTTTGGTAGCTGGCGATAAAAAGACTAACGTCGGTGGGTCAGAAATTTATTACAACA
TGGATAATCGTCTCCACCAAATAGATGGAAGCAATACAATATTTGTACGTGGCGATGAAA
CTAAGACAGTTGAAGGTAATGGAACTATCCTAGTCAAAGGTAATGTTACTATTGTAGTTG
AAGGTAATGCTGACATTACAGTTAAAGGAGATGCTACCACTTTAGTTGAAGGAAATCAGA
CTAATACAGTAAATGGAAATCTTTCTTGGAAAGTTGCTGGGACAGTTGATTGGGACGTTG
GTGGTGATTGGACAGAAAAAATGGCATCTATGAGTTCTATTTCATCTGGTCAATACACAA
TTGATGGATCGAGGATTGACATTGGCTAATATACTTCCAATGAGCGCTGATTTAGGAGAA
TCCATGGAAGGTTCTTCTATCGACGTCACCTTTACCGCTCAATTAGAAACAGGTGAAACG
TTAGTATCTATAAATATAACTAGTTACGAAGAAACTCCTGGGGTTTTAGTAGAAGAAAAT
CGTTTATATGGAACGTATGAATCTGTATTTGGATTTGGAAATGATGCATTGAAATATCGT
TTAGATAATGAATTTAAAACTGCTGCTTCATGGGAAGACCTTCCAACTGATTCTAATACT
CAGTTATATTTATGGAAAGCTCCTCAAAACCTCCAGAAGACATTCACTTACGAAGTAACA
TTAATATATGACTATCAAGAACAAGGTGAATCTGGAGGTTCTGGTAGTAATTCTAGGTCA
TCTTCTGATACTACTGAACCGACAGATCCTCCTGCTCCAGTAAGAAAAACTCTCGTTAAA
AATTATACTAAAACTATAGTTGGAAATTGGAGTCGTTGGGCTAATAAATTGAGAAGCTAT
GTGTATGAGAGGCCATAAATGTCAGGATTAAGTTATGATAAGTGTGTTACTGCTGGCCAT
GAAGCGTGGCCTCCAACAGTTGTGAATGCTACACAAAGTAAAGTATTCACTGGAGGAATT
GCTGTTCTAGTAGCAGGCGATCCAATTACAGAACATACAGAAATTAAAAAGCCATATGAA
ACACATGGCGGAGTGACACAACCTAGAACTTCTAAGGTATATGTCACTGGAAAGAAAGCT
GTTCAAATGGCTGATCCAATATCATGCGGTGATACTGTGGCTCAGGCATCATCTAAAGTA
TTCATTAAATAGGATTTAAAATGGCAAATACCCCTGTAAATTATCAATTAACAAGAACAG
CAAATGCTATTCCCGAGATATTCGTCGGGGGTACATTTGCTGAAATAAAACAAAACCTCA
TTGAATGGCTTAATGGCCAAAATGAATTTTTGGATTATGATTTTGAAGGCTCAAGATTAA
ACGTTCTGTGTGACCTTTTGGCTTATAATACGCTGTACATTCAGCAGTTTGGTAATGCTG
CTGTGTATGAAAGCTTTATGCGTACTGCTAACTTACGAAGTTCAGTTGTTCAAGCTGCAC
AAGATAACGGATATTTACCTACTTCAAAATCCGCTGCGCAGACCGAAATTATGTTAACAT
GCACCGACGCATTGAATAGGAATTACATTACTATTCCTCGCGGAACTCGCTTTTTAGCAT
ATGCAAAAGATACTTCTGTTAATCCATATAACTTCGTTTCTACCGAAGACGTTATTGCCA
TTCGTGATAAAAATAACCAATATTTTCCGCGTTTAAAATTGGCCCAGGGACGTATAGTAA
GAACTGAAATCATTTATGATAAATTAACACCTATTATCATTTATGATAAAAATATTGATA
GAAACCAGGTTAAATTATATGTTGATGGAGCGGAATGGATTAACTGGACAAGAAAGTCAA
TGGTTCATGCTGGTTCAACATCAACGATTTATTATATGCGTGAAACTATTGATGGAAACA
CTGAATTCTATTTTGGTGAAGGTGAAATTTCTGTTAATGCTTCTGAAGGAGCTTTGACCG
CTAATTATATTGGAGGTCTTAAACCTACCCAGAACTCTACGATTGCTATTGAGTATATCA
GTACTAACGGTGCAGATGCGAATGGTGCAGTCGGATTTTCATATGCAGATACATTAACAA
ATATAACTGTCATCAATATTAATGAAAATCCAAACGATGACCCAGATTTTGTTGGGGCAG
ATGGCGGCGGCGATCCAGAAGATATTGAACGTATTCGCGAATTGGGTACTATTAAACGCG
AAACCCAGCAACGCTGCGTAACTGCGACTGACTATGATACATTCGTTTCAGAGAGATTTG
GTTCTATTATTCAAGCAGTTCAGACGTTCACTGATTCTACTAAACCCGGTTATGCATTTA
TTGCTGCTAAACCTAAATCAGGTCTGTATTTAACTACCGTACAGCGCGAAGATATTAAAA
ATTATCTCAAAGACTATAATTTAGCTCCTATTACACCATCAATTATTTCTCCTAATTATC
TTTTTATTAAGACTAATTTAAAAGTCACATATGCTTTAAATAAGCTGCAAGAATCCGAAC
AGTGGCTCGAAGGTCAAATAATTGATAAAATTGATCGTTATTATACCGAAGATGTAGAAA
TTTTTAACTCATCTTTCGCTAAATCTAAGATGTTGACATATGTAGATGATGCAGATCATT
CTATCATTGGTTCATCAGCGACAATTCAAATTGTTCGTGAAGTACAAAACTTCTATAAAA
CGCCTGAAGCAGGTATTAAATACAATAATCAAATAAAAGATCGTTCTATGGAATCTAATA
CGTTTTCATTTAATTCTGGACGAAAGGTTGTAAATCCTGATACTGGTTTAGAAGAAGATG
TATTATATGACGTTCGCATAGTATCAACAGACCGAGATTCTAAAGGAATTGGTAAAGTTA
TTATTGGTCCATTTGCTTCTGGCGATGTTACAGAAAATGAAAACATTCAGCCGTATACAG
GCAACGATTTTAACAAATTAGCAAATTCTGATGGACGCGACAAATACTATGTTATCGGTG
AAATAAATTATCCAGCTGATGTGATTTATTGGAATATCGCTAAAATTAATTTAACATCTG
AAAAATTTGAAGTTCAGACCATCGAATTATATTCTGACCCAACCGATGATGTTATTTTTA
CTCGCGACGGTTCACTGATTGTATTTGAAAATGACTTACGTCCACAATACTTAACTATCG
ATTTGGAGCCTATATCACAATGACAGTAAAAGCACCTTCAGTCACTAGTCTCAGAATTTC
CAAGTTATCCGCAAATCAGGTGCAAGTACGCTGGGATGACGTTGGTGCTAATTTCTACTA
TTTTGTAGAAATCGCTGAGACAAAAACAAACTCGGGGGAAAATCTCCCGAGTAATCAATA
CAGATGGATTAACTTAGGATATACTGCAAATAACAGCTTCTTTTTTGATGATGCTGACCC
ATTGACATCATACGTTATTAGAGTAGCTACAGCTGCACAAGATTTTGAGCAGTCTGATTG
GATTTATACCGAAGAGTTTGAAACTTTTGCTACAAATGCTTATACATTTCAAAACATGAT
TGAAATGCAATTAGCCAATAAATTCATTCAGGAAAAATTTACTCTTAATAATTCTGATTA
TGTCAATTTTAATAATGACACTATAATGGCTGCATTGATGAATGAATCTTTTCAATTCAG
TCCATCATATGTTGATGTTTCATCAATAAGTAATTTTATTATTGGTGAAAATGAGTATCA
TGAAATACAGGGTTCTATTCAGCAAGTATGTAAGGATATTAACCGAGTTTATTTGATGGA
ATCAGAAGGGATTCTATACCTTTTTGAGCGCTATCAACCTGTAGTTAAAGTATCCAATGA
TAAAGGACAAACTTGGAAAGCTGTAAAGCTCTTCAATGACCGTGTAGGATATCCTTTATC
TAAGACAGTATATTACCAATCTGCGAACACAACATACGTTCTAGGATACGACAAGATTTT
CTATGGCCGTAAATCGACTGATGTTAGATGGTCAGCTGATGATGTCAGATTTAGTTCTCA
GGATATAACATTTGCTAAACTTGGCGACCAGTTACATTTAGGATTTGATGTAGAAATTTT
TGCCACTTACGCGACTTTGCCAGCGAATGTATACCGCATTGCAGAAGCTATTACTTGCAC
CGATGATTACATTTACGTTGTCGCCAGAGACAAAGTTAGATACATAAAAACGAGTAATGC
ACCTATAGATTCTGATCCATTATCTCCAACATATTCGGAAAGACTTTTTGAACCTGATAC
CATGACTATAACCGGAAATCCTAAAGCAGTATGTTATAAAATGGATTCTATCGGTGATAA
AGTTTTTGCTCTTATTATTGGTGAAGTTGAAACATTAAATGCTAATCCTAGAACATCAAA
AATAATTGATTCCGCTGATAAAGGAATATATGTTTTAAATCATGACGAAAAAACATGGAA
AAGAGTTTTTGGTAATACCGAAGAAGAAAGAAGACGTATTCAACCCGGATATGCGAATAT
GTCAACTGACGGTAAATTAGTTTCTCTGTCTTCGAGTAATTTTAAATTTTTAAGTGATAA
TGTTGTTAATGACCCTGAAACTGCAGCAAAATATCAGTTAATTGGTGCTGTCAAATATGA
ATTTCCTCGTGAATGGCTAGCTGATAAGCATTATCATATGATGGCATTTATAGCAGATGA
AACATCTGATTGGGAGACTTTTACTCCTCAACCAATGAAATACTACGCAGAACCATTCTT
TAACTGGTCTAAAAAATCTAACACACGTTGTTGGATAAACAACTCTGATAGAGCTGTAGT
AGTTTATGCTGATTTAAAATACACTAAAGTTATAGAAAATATTCCGGAAACATCACCAGA
TAGATTAGTTCATGAATACTGGGATGATGGTGATTGTACTATAGTAATGCCAAATGTTAA
GTTCACCGGATTTAAAAAATATGCATCAGGAATGCTTTTCTATAAAGCCTCCGGTGAAAT
AATTTCTTACTATGATTTTAACTATCGTGTGAGAGATACAGTAGAAATTATTTGGAAGCC
AACTGAAGTATTTTTAAAAGCATTTTTACAAAACCAAGAGCATGAGACTCCTTGGTCACC
AGAAGAAGAGCGTGGATTAGCTGACCCTGATTTAAGACCATTGATTGGTACAATGATGCC
TGATTCTTATTTGTTACAGGATTCGAATTTTGAAGCATTTTGCGAAGCATATATTCAGTA
TCTTTCCGATGGATATGGAACTCAATACAATAATTTACGGAATCTTATTCGTAATCAGTA
TCCACGAGAAGAGCATGCATGGGAATATTTGTGGTCAGAGATATATAAAAGAAACATCTA
TTTAAATGCTGACAAACGTGATGCTGTTGCAAGATTTTTCGAATCACGTAGCTATGATTT
TTATTCTACTAAAGGAATTGAAGCATCATACAAATTTCTTTTTAAAGTTCTTTATAATGA
AGAGGTTGAAATTGAAATTGAATCTGGGGCTGGTACTGAATATGATATAATCGTTCAATC
TGATTCTTTGACTGAAGACTTAGTAGGACAAACGATTTATACAGCTACAGGAAGATGTAA
TGTTACTTATATAGAAAGAAGCTATTCTAATGGTAAATTGCAATGGACCGTAACTATTCA
TAATCTTTTGGGACGATTAATTGCTGGCCAAGAAGTTAAAGCAGAAAGACTCCCTAGTTT
TGAAGGCGAAATTATTCGTGGGGTTAAAGGAAAGGATTTGCTTCAAAACAATATAGACTA
TATTAATAGAAGTAGATCATACTATGTAATGAAAATTAAATCCAATTTACCTTCTTCCCG
CTGGAAATCTGATGTTATTCGTTTTGTTCATCCAGTAGGATTTGGATTTATAGCAATTAC
ACTTTTAACAATGTTTATTAATGTTGGTTTAACTCTTAAACATACAGAGACTATAATTAA
TAAATACAAAAACTATAAATGGGATTCTGGATTGCCTACTGAATATGCTGATAGAGTAGC
TAAATTAACTCCAGCTGGTGAAATTGAGCATGATTCAGTAACAGGCGAAGCAATTTATGA
GCCTGGCCCAATGGCTGGTGTAGAATATCCTCTTCCTGATGACTATAATGCTGAAAATAA
TAATTCAATATTTCAAGGTCAATTGCCGTCTGAACGACGTAAATTAATGAGTCCTTTATT
TGATGCATCTGGAACAACATTTGCGCAATTTAGAGATTTAGTTAATAAACGTCTAAAAGA
CAATATAGGAAATCCAAGAGACCCTGAAAATCCAACACAGGTTAAAATAGATGAATGATT
CAAGTGTTATCTATCGTGCGATAGTTACTTCAAAATTTAGAACAGAAAAAATGTTGAATT
TTTATAATTCAATTGGAAGTGGTCCGGATAAAAACACTATCTTTATCACATTTGGAAGAT
CAGAACCGTGGTCATCAAATGAAAATGAGGTGGGCTTTGCCCCACCTTATCCAACCGATT
CTGTATTAGGCGTAACTGACATGTGGACGCATATGATGGGAACAGTAAAGGTTCTTCCAT
CAATGCTTGATGCTGTTATTCCTCGCAGAGATTGGGGAGATACTAGATATCCGGATCCAT
ACACATTTAGAATTAACGATATTGTAGTGTGTAACTCAGCTCCTTACAACGCTACTGAAT
CTGGCGCGGGTTGGTTAGTGTATCGTTGTTTAGATGTTCCTGATACCGGAATGTGTTCAA
TAGCATCTTTAACTGATAAGGATGAATGCCTTAAATTAGGCGGAAAATGGACACCTTCTG
TTAGGTCAATGACTCCTCCTGAAGGTCGAGGAGATGCTGAAGGAACAATTGAACCTGGGG
ACGGATATGTTTGGGAATATCTTTTTGAGATTCCGCCTGATGTATCTATAAATAGATGCA
CGAATGAATATATCGTGGTTCCTTGGCCTGAGGAATTAAAAGAAGACCCGACTAGATGGG
GATATGAAGATAATCTCACTTGGCAACAAGATGATTTTGGATTAATTTACCGTGTTAAGG
CAAATACTATCCGTTTTAAAGCATATTTAGATTCAGTTTATTTTCCTGAAGCTGCATTGC
CAGGAAATAAAGGATTTAGACAAATATCAATAATCACGAATCCTCTTGAAGCTAAAGCTC
ATCCAAATGACCCAAACGTTAAAGCTGAAAAGGATTATTATGACCCAGAAGATTTAATGA
GGCATTCGGGTGAAATGATTTATATGGAAAATAGGCCACCTATTATTATGGCAATGGATC
AGACAGAAGAAATCAATATTCTGTTTACATTTTAAATTAAGGGAGCCCATGGGCTCCCTT
TTTCTTTATAAATACTATAAACTCATAAGGAAACCGCTATGTTCATTCAAGAACCAAAGA
AATTGATTGATACCGGCGAAATTGGTAACGCTTCTACTGGTGATATCTTATTCGACGGTG
GTAATAAAATTAATAGTGATTTTAATGCAATTTATAATGCGTTTGGCGATCAACGTAAAA
TGGCAGTAGCAAATGGCACCGGAGCAGATGGTCAAATTATCCATGCTACTGGATATTATC
AAAAACACTCTATTGCAGAGTATGCAACTCCAGTAAAAGTTGGCACTAGGCATGATATTG
ATACCTCTACTGTAGGTGTTAAAGTTATCATTGAAAGAGGCGAACTCGGCGATTGTGTTG
AATTCATTAATTCTAACGGATCAATATCAGTTACTAATCCTTTGACAATTCAAGCTATTG
ATTCAATTAAAGGTGTTTCAGGTAATTTAGTAGTAACTAGCCCATATAGTAAAGTTACTT
TACGCTGTATTTCATCTGATAATTCTACGTCGGTTTGGAATTATTCTATTGAAAGTATGT
TTGGACAAAAGGAATCACCAGCTGAAGGTACATGGAATATTTCTACATCTGGATCAGTTG
ATATTCCACTATTTCATCGCACTGAATACAATATGGCTAAATTACTAGTTACATGCCAAT
CGGTAGATGGAAGAAAAATTAAAACAGCAGAAATAAATATTCTTGTGGATACTGTCAATT
CAGAGGTAATTTCTTCTGAATATGCTGTCATGCGAGTTGGGAATGAAACCGAAGAAGACG
AAATCGCTAATATTGCATTTAGTATTAAAGAAAATTACGTAACGGCGACTATAAGTTCTT
CAACTGTCGGTATGAGAGCAGCAGTTAAAGTTATCGCTACGCAGAAAATCGGGGTGGCTC
AATAATGAAACAAAATATTAATATCGGTAATGTTGTAGATGATGGTACCGGTGACTACCT
GCGTAAAGGTGGTATAAAAATAAATGAAAACTTTGATGAGCTTTATTATGAACTCGGTGA
TGGTGATGTTCCATATTCAGCCGGTGCCTGGAAAACTTATAATGCTTCATCAGGACAAAC
ATTAACAGCAGAATGGGGAAAATCATACGCTATTAATACATCTTCTGGAAGAGTGACTAT
AAATCTTCCAAAGGGTACAGTTAATGATTACAACAAGGTAATTAGAGCTAGAGACGTATT
TGCTACATGGAACGTCAACCCAGTTACACTAGTAGCTGCTTCCGGCGATACGATTAAAGG
GTCTGCAGTACCAGTTGAAATTAATGTTCAATTCAGCGATTTAGAACTAGTGTATTGTGC
CCCAGGACGTTGGGAATATGTCAAAAATAAACAAATTGACAAAATTACCAGTTCAGACAT
TAGTAATGTAGCTCGTAAAGAATTTTTAGTCGAAGTCCAAGGACAAACGGACTTTTTAGA
TGTTTTCCGTGGAACTAGTTATAATGTAAATAACATCAGAGTAAAACATCGCGGTAACGA
ATTGTATTACGGTGATGTGTTTAGCGAAAACAGCGATTTTGGCTCTCCAGGTGAAAATGA
AGGAGAACTGGTTCCTCTTGATGGATTTAATATTCGATTAAGACAGCCTTGTAATATTGG
TGACACTGTTCAAATTGAAACATTTATGGATGGTGTATCGCAGTGGAGAAGTTCATATAC
AAGACGTCAAATTAGATTGTTAGATTCAAAATTAACGTCAAAAACTTCTTTAGAAGGAAG
CATTTACGTTACTGATTTATCAACAATGAAATCAATTCCATTTTCTGCTTTTGGATTAAT
TCCAGGAGAACCTATTAATCCCAACTCTCTTGAGGTTCGTTTTAATGGGATTTTGCAGGA
ATTGGCTGGAACAGTTGGAATGCCATTATTTCATTGTGTTGGTGCCGATTCAGACGATGA
AGTAGAATGCTCTGTTTTAGGTGGAACTTGGGAACAATCTCATACCGATTATTCAGTTGA
AACTGATGAAAACGGCATACCGGAAATTTTACATTTCGATAGAGTATTTGAGCATGGTGA
CATTATCAATATCACCTGGTTTAATAATGATTTGGGTACATTATTAACAAAAGATGAGAT
TATTGATGAAACTGATAATCTCTATGTATCGCAAGGACCAGGGGTAGATATTTCCGGTGA
TGTAAATTTAACAGACTTCGATAAAATTGGTTGGCCAAATGTAGAAGCGGTTCAATCTTA
TCAACGCGAATTTAATGCTGTTTCAAATATCTTTGATACGATTTATCCTATTGGAACTAT
ATATGAAAACGCTGTTAATCCAAACAACCCTGTTACATATATGGGATTCGGCTCATGGAA
ATTATTTGGGCAAGGAAAAGTTTTAGTTGGATGGAATGAAGATATTTCGGACCCTAACTT
TGCTCTAAATAACAACGATTTAGATTCGGGTGGAAATCCTTCACATACTGCAGGTGGAAC
AGGCGGTTCTACTTCTGTTACATTGGAAAATGCTAATCTTCCTGCAACTGAAACAGATGA
AGAAGTTCTAATAGTTGATGAAAATGGATCAGTCATTGTTGGTGGGTGTCAATACGATCC
AGATGAATCCGGTCCAATTTACACTAAATACCGTGAAGCTAAAGCATCTACTAACTCTAC
TCACACTCCGCCAACATCAATAACTAACATTCAACCATATATTACAGTTTATCGTTGGAT
AAGGATTGCATAATGAGTTTACTTAATAATAAAGCGGGAGTTATTTCCCGCTTAGCCGAT
TTTCTTGGTTTTAGACCTAAAGCTGGTAATATAGATGTAATGAATCGTCAATCAGTTGGG
TCAGTGACAATTTCTCAATTAGCGAAAGGATTCTATGAACCAAACGTAGAATCAGCTATT
AATGATGTTCATAATTTTTCTATAAAAGACATTGGTACAATTATTACTAATAAAACTGGT
GTTTCTCCTGAGGGTGTTTCTCAAACTGATTATTGGGCATTTTCTGGAACTGTAACAGAC
GATTCTCTTCCTCCGGGTTCTCCTGTCACAGTATTAGTATTTGGTCTTCCAGTTTCAGCA
ACAACTGGAATGACGGCAATTGAGTTTGTTGCAAAAGTTCGTGTTGCCCTTCAAGAAGCT
ATTGCGTCATTTACTGCTATCAACTCATATAAAGACCATCCGACAGATGGTAGTAAATTA
GAAGTTACTTATTTAGATAATCAAAAACATGTATTAAGCACATATTCTACATATGGAATA
ACTATTTCACAGGAAATTATTTCTGAGTCTAAACCTGGCTACGGTACATGGAATTTATTA
GGCGCACAAACTGTAACTTTAGATAATCAGCAGACTCCTACAGTACTTTATCATTTTGAG
AGAACAGCATGAGTAATAATACATATCAACACGTTTCTAATGAATCTCGTTATGTAAAAT
TTGATCCAACTGGTACAAATTTTCCGGAAGAGATTACTGATGTTCAGGCTGCTATAGCAG
CCATTTCTCCTGCTGGCGTAAATGGAGTTCCTGATGCATCGTCAACAACAAAGGGAATTT
TATTTCTTGCCACTGAACAGGAAGTTATAGATGGAACTAATAATACCAAAGCAGTTACAC
CGGCAACGTTGGCAACAAGATTATCATATCCAAATGCTACCGAAGAAGTTTATGGACTTA
CACGTTATTCAACAGACGATGAAGCTGTAGCTGGTGTTAATAATGAATCATCTATAACTC
CGGCTAAATTTACTACAGCAATTAATCACATTTTTGATACACGAATTTCTTCTGAAAGTT
CACTTGGTGTTATTAAGTTATCATCTCTACCGCAAGCATTAGCTGGTGCAGATGATACTA
CTGCAATGACTCCATTAAAAACGCAGCAGTTAGCTATTAAATTAATTGCGCAAATTGCTC
CTTCTGAAACCACAGCTACCGAATCGGACCAAGGTGTTGTTCAATTAGCAACAGTAGCGC
AGGTTCGTCAGGGAACTTTAAGAGAAGGCTATGCAATTTCTCCTTATACGTTTATGAATT
CATCTGCTACTGAAGAATATAAAGGCGTAATTAAATTAGGAACACAATCAGAAGTTAACT
CGAATAATGCTTCTGTTGCTGTTACTGGTGCAACTCTTAATGGTCGTGGTTCTACAACGT
CAATGAGAGGCGTAGTTAAATTAACTACAACTGCCGGCTCACAGAGTGGAGGTGATGCTT
CGTCAGCACTAGCTTGGAATGCTGACGTTATCCATCAAAGAGGCGGTCAAACTATTAATG
GAACACTCCGCATTAATAACACGCTTACAATAGCTTCAGGTGGAGCAAATATTACCGGAA
CAGTTAACATGACCGGTGGTTATATTCAAGGTAAGCGTGTCGTAACACAAAATGAAATTG
ATAGAACTATTCCTGTCGGAGCTATTATGATGTGGGCCGCTGATAGTCTTCCTAGTGATG
CTTGGCGTTTTTGCCACGGTGGAACTGTTTCAGCATCAGATTGTCCATTATATGCTTCTA
GAATTGGAACAAGATATGGCGGAAGTTCATCAAATCCTGGATTACCTGATATGCGTGGTC
TTTTTGTTCGCGGCTCTGGTCGTGGCTCTCACTTAACAAATCCAAATGTTAATGGTAATG
ACCAGTTTGGTAAGCCTAGATTAGGTGTAGGTTGTACTGGTGGATATGTTGGTGAAGTAC
AGAAACAACAGATGTCTTATCATAAACATGCTGGCGGATTTGGTGAGCATGATGATTCTG
GAGCATTCGGTAATACCCGTAGATCAAATTTTGTCGGTACACGTAAAGGACTTGACTGGG
ATAACCGTTCATACTTCACCAATGACGGATACGAAATTGACCCGGCATCACAACGAAATT
CCAAATATACATTAAATCGTCCTGAATTAATTGGAAATGAAACACGTCCATGGAACATTT
CTTTAAACTACATAATTAAGGTAAAAGAATGACAGATATTGTACTGAATGACTTACCATT
CGTTGACGGCCCTCCTGCGGAGGGCCAGAGCCGCATTTCCTGGATTAAAAACGGCGAAGA
AATATTAGGAGCTGACACACAGTATGGAAGCGAAGGTTCAATGAATAGACCTACAGTTTC
TGTACTAAGAAATGTTGAAGTTCTTGATAAAAACATTGGAATACTTAAAACATCTTTAGA
AACCGCAAATAGTGATATTAAAACCATCCAGGGCATCTTAGATGTATCTGGTGATATAGA
AGCTTTAGCACAAATAAGTATCAATAAAAAGGATATTTCTGACCTCAAAACGCTAACCAG
TGAACATACAGAAATATTAAATGGAACTAATAGTACAGTTGACAACATTCTTGCTGATAT
TGGTCCATTTAACTCCGAGGCCAACTCTGTATACAGAACGATCAGAAATGATTTACTGTG
GATAAAGCGTGAACTTGGACAATACACTGGTCAAGATATTAATGGTCTTCCTGTTGTAGG
AAATCCTAGTAGTGGAATGAAGCATCGCATTATTAATAATACTGATGTCATTACTTCGCA
GGGAATACGTTTAAGTGAATTAGAAACAAAATTTATTGAATCTGATGTAGGTTCTTTGAC
CATTGAAGTTGGTAATCTTCGCGAAGAGCTTGGACCGAAACCACCGTCTTTTTCACAAAA
CATTTACAGCCGCTTAAATGAAATTGACACTAAACAGACAACAGTTGAGTCTGACATTAG
TGCTATTAAGACCTCAATAGGATATCCAGGAAATAATTCGATTATTACGAGTGTTAATAC
AAACACTGATAATATTGCATCTATTAATTTAGAGCTAAATCAAAGTGGAGGTATTAAACA
GCGTTTAACCGTTATTGAAACTTCTATTGGTTCAGATGATATTCCTTCGAGTATTAAAGG
CCAAATCAAAGATAATACAACTTCAATCGAATCTTTAAATGGAATCGTCGGTGAAAACAC
TTCATCTGGTTTAAGAGCGAATGTTTCGTGGTTAAACCAAATTGTTGGAACTGATTCTAG
CGGTGGACAACCTTCTCCTCCTGGGTCTCTTTTAAACCGAGTTTCTACAATTGAAACTTC
TGTTTCAGGCTTGAATAACGATGTTCAAAACCTACAAGTAGAGATTGGTAATAACAGCAC
AGGAATTAAAGGACAAGTTGTAGCGTTAAATACTTTAGTAAATGGAACTAATCCAAACGG
TTCAACTGTTGAAGAACGCGGATTAACCAATTCAATAAAAGCTAACGAAACTAACATTGC
ATCAGTTACACAAGAAGTGAATACAGCTAAAGGCAATATATCTTCTTTACAAGACGATGT
TCAAGCTCTCCAAGAAGCCGGTTATATTCCTGAAGCTCCAAAAGATGGCCAAGCTTACGT
TCGTAAAGACGGCGAATGGGTATTGCTTTCTACCTTTTTATCACCAGCATAACATGGGGC
CGCAAGGCCCCAAAGGATTTTAAATGTCAGGATATAATTCTCAGAATCCAAAGGAACTCA
AAGATGTCATTCTAAGACGTTTAGGGGCTCCAATTATTAATGTTGAGTTAACACCCGATC
AAATTTACGATTGTATCCAGCGTGCCCTAGAATTATACGGTGAATACCATTTTGATGGAC
TTAACAAAGGTTTTCACGTATTTTACGTAGGGGACGATGAAGAAAAGTATAAAACTGGAG
TCTTCGATTTAAGAGGTTCTAACGTATTTGCAGTAACCCGCATTTTACGCACAAATATTG
GGTCAATAACGTCAATGGACGGAAACGCTACATATCCATGGTTTACTGACTTTCTTTTAG
GAATGGCTGGTATTAATGGCGGAATGGGAACGTCTTGTAATAGATTTTATGGACCAAATG
CCTTTGGTGCCGATTTGGGGTATTTTACTCAACTTACCAGTTATATGGGAATGATGCAAG
ATATGCTCTCTCCTATTCCAGACTTTTGGTTTAATTCAGCAAATGAACAACTCAAAGTCA
TGGGAAACTTCCAAAAATATGATTTAATTATCATAGAAAGCTGGACTAAATCATACATTG
ATACTAATAAAATGGTTGGAAATACAGTAGGATATGGAACAGTCGGTCCACAAGATAGCT
GGTCATTATCTGAACGTTATAATAACCCAGACCACAATTTAGTGGGTCGTGTTGTTGGTC
AAGACCCAAATGTTAAGCAAGGTGCTTACAATAATCGTTGGGTGAAAGACTATGCAACAG
CTTTAGCTAAAGAATTGAATGGTCAAATTTTAGCACGCCACCAAGGTATGATGCTTCCTG
GCGGTGTTACGATTGATGGACAACGCTTAATAGAAGAAGCTCGATTAGAAAAAGAAGCAC
TGCGCGAAGAATTATACTTACTTGACCCTCCATTTGGAATTTTGGTAGGTTAATATGGCT
ACTTATGATAAAAATCTTTTTGCTAAATTGGAAAACCGCACAGGTTATTCTCAGACCAAT
GAAACTGAAATATTAAATCCTTATGTAAATTTCAATCATTATAAAAACAGCCAAATATTA
GCTGATGTATTAGTAGCTGAAAGCATTCAAATGCGAGGGGTAGAATGCTATTATGTTCCA
AGAGAGTATGTTTCCCCTGATTTGGTATTCGGCGAAGACTTAAAAAATAAATTTACTAAA
GCTTGGAAATTTGCTGCGTATTTAAATTCATTTGAAGGATATGAAGGAGCTAAATCGTTC
TTTAGTAACTTTGGTATGCAAGTACAAGACGAAGTGACTTTATCTATTAACCCAAATTTA
TTTAAGCATCAAGTTAACGGAAAAGAACCGAAAGAAGGCGATTTGATATATTTTCCTATG
GATAACAGCTTATTTGAAATTAACTGGGTTGAACCATATGATCCATTTTATCAATTAGGC
CAAAACGCTATTCGTAAAATTACAGCAGGTAAATTCATTTATTCTGGAGAAGAAATTAAT
CCAGTTCTACAGAAAAATGAAGGAATTAACATTCCAGAATTTAGTGAATTAGAACTAAAT
CCTGTTCGCAATCTTAACGGTATTCATGATATTAATATTGATCAGTATGCTGAAGTAGAT
CAAATTAATTCTGAAGCTAAAGAATATGTTGAACCTTATGTTGTTGTCAATAACAGAGGC
AAATCTTTTGAATCTAGCCCATTTGATAATGATTTCATGGATTAATAAATATTATAAACT
AATTAAAGCCCAGATTAGGAGAAATCATGTTTGGCTATTTTTATAATTCGTCTTTTAGAC
GATATGCTACCTTGATGGGCGATTTGTTTTCAAATATCCAAATCAAACGTCAGTTAGAAT
CTGGTGATAAGTTTATACGTGTTCCTATTACATATGCGTCAAAGGAACACTTTATGATGA
AATTGAATAAATGGACATCAATAAATTCACAAGAAGATGTAGCTAAAGTTGAAACTATTC
TACCTCGTATAAATTTACATTTAGTTGATTTTAGCTATAATGCTCCATTTAAAACAAACA
TTTTAAATCAGAATTTACTGCAAAAAGGTGCAACTTCTGTAGTATCGCAGTATAATCC

In another aspect, the invention provides a PG-I1 bacteriophage targeting Staphylococcus aureus. In certain embodiments, PG-I1 binds a molecule of peptidoglycan. In certain embodiments, the PG-I1 bacteriophage has a genome comprising at least one nucleic acid sequence selected from the group consisting of SEQ ID NOs: 10-12:

SEQ ID NO: 10
TCAGTGTCTACTGATTTAGAAACGTAACCTTCTACTTTCTTGCTCTCCGTTTGTAGATGT
TACTGATTTTTCTACAGCTTCTTCATCAGTGTCTACTGATTTAGAA
SEQ ID NO: 11
TTCTAAATCAGTAGACACTGAAGAACAAGCTGAAACTGGTGAAGCAAAATCAGAAGAAGC
TGAAGAAGTACAAGAAGATAACACATTTAAAGGATTAAGTCAAGAAGAACGAACTAAGTT
CATGGATTCTTACAAAGCACAAGCTAAAGACCCTAGAGCTTCTAAACATGACTTACAATC
AGCTTACCAATCTTACTTGAACATTAACACTGACCCTACTAATGCATCAGAGAAAGATAT
TAAAACTGTAAAAGACTTTGCACAAATTTAATTAATGCACAAAGTTGTGTTATATTATAC
GGTGTAACTAAAGAATATAAATAGGGTACATTTTACTGTACCCCACATAAAATAAAAAAG
AACACAAAATGAAAGGTGATAAATTTATATGACTATCGAAAAGAACCTGTCAGACGTTCA
ACAAAAGTACGCTGACCAATTCCAAGAAGACGTAGTAAAGTCATTCCAAACTGGTTATGG
AATCACTCCTGATACACAAATTGACGCAGGAGCTTTACGTAGAGAAATTTTAGATGACCA
AATCACAATGTTAACATGGACTAATGAAGACTTAATCTTCTATCGTGATATCTCACGCCG
TCCTGCTCAATCTACAGTAGTAAATACGACCAATATTTACGTCATGGTAACGTAGGTCAC
TCTCGTTTCGTTAAAGAAATCGGAGTAGCACCAGTATCTGACCCAAATATCCGTCAAAAA
ACTGTATCAATGAAATACGTTTCTGATACTAAAAATATGTCAATTGCATCAGGTTTAGTA
AATAACATTGCTGACCCATCACAAATCCTTACAGAAGATGCTATCGCAGTTGTTGCAAAA
CAATTGAGTGGGCTTCATTCTACGGTGACGCTTCATTAACTTCTGAAGTTGAAGGTGAAG
GTCTAGAGTTTGATGGTTTAGCTAAATTAATTGACAAAAATAACGTAATTAACGCTAAAG
GTAATCAATTAACTGAGAAACACTTAAATGAGGCGGCGGTACGTATCGGTAAAGGTTTCG
GTACAGCTACAGATGCTTACATGCCTATCGGTGTACACGCAGACTTCGTTAACTCAATCT
TAGGTCGTCAAATGCAATTAATGCAAGACAACAGCGGTAACGTTAACACTGGTTACAGCG
TAAATGGTTTCTACTCATCTCGTGGATTCATTAAATTACATGGTTCTACAGTAATGGAAA
ATGAACTAATCTTAGATGAATCATTACAACCATTACCAAATGCTCCACAACCTGCTAAAG
TTACAGCTACTGTTGAAACTAAGCAAAAGGTGCTTTTGAAAAATGAAGAAGACCGTGCAG
GATTATCATATAAAGTAGTAGTTAACTCAGATGACGCTCAATCAGCTCCTTCTGAAGAAG
TAACAGCTACAGTATCTAACGTAGACGATGGTGTTAAACTTTCAATTAATGTTAACGCTA
TGTACCAACAACAACCACAATTCGTTTCTATCTACCGTCAAGGTAAAGAAACAGGTATGT
ACTTCCTAATCAAACGTGTACCAGTTAAAGATGCACAAGAAGACGGAACAATCGTATTCG
TAGATAAGAACGAAACATTGCCTGAAACAGCAGACGTATTTGTTGGTGAAATGTCACCAC
AAGTAGTTCACTTATTCGAATTACTTCCAATGATGAAATTACCATTAGCTCAAATTAATG
CTTCTATTACATTTGCAGTATTATGGTATGGTGCATTAGCATTACGATGCTCTAAAAAAA
TGGGCTCGTATTAAAACGTTCGTTATATCGCAGTTTAATAGAATAAGAAAAACTGAATAC
AAGAGAATAGGGATAAACTTAGGGTTTATCCCTTTTTTATTAAATAAACTTGAAGGGATT
TAATAAATATGTTATACTATAAGAAACTATTAGATAAAAAAATGGCTACTGTTTATGGTA
CAGTGGAGATTGACAAAGATGGAGTAGTCAAAGGATTAACCAAGAACAAGAAAAGAATTT
GCCAATGTTCCAGGTTTTGAATTTGAAGAAGAAAAGAAAACTACTAGAAAACAATCAGCT
TCTACTAGTAAAGAAGAAGAGCCTAAGGAAGAGGAAAAGCCTCTACTAGAAAAACTACAA
ATACTACTAGAAAATCTACAGCACGTAAAACAACAGCCAAAAAAAAGATGAAAATAAGTA
AAGGGTGAATTAAATGGTTAACTCAATGTTTGGAGGGGACTTAGACCCTTATGAAAATCA
TTAAACTATGAATATCCTTATCATCCTAGTGGTAATCCTAAACACATAGATGTAAGTGAG
ATAGATAATTTAACATTAGCTGATTATGGATGGTCACCGGATGCAGTTAAAGCATATATG
TTCGGTATTGTAGTTCAAAATCCTGATACAGGACAGCCTATGGGTGACGAGTTCTATAAC
CATATATTGGAAAGAGCGGTAGGTAAAGCTGAAAGAGCATTAGATATATCTATACTACCT
GACACTCAACATGAGATGAGAGATTATCATGAGACAGAGTTTAATAGTTACATGTTTGTA
CATGCTTACAGAAAACCTATATTACAGGTAGAGAACTTACAGCTACAGTTTAATGGTAGA
CCCATATATAAATACCCTGCTAACTGGTGGAAAGTAGAGCATCTAGCAGGACATGTTCAA
TTATTCCCTACAGCACTTATGCAAACAGGACAATCAATGTCATACGATGCAGTATTCAAT
GGATACCCCTCAATTAGCAGGTGTATACCCACCATCAGAGCAACATCATTTGCACCTCCT
CAAATGATACGATTAGAATATGTATCAGGTATGCTTCCACGTAAAAAGCAGGAAGAAATA
AACCTTGGGAAATGCCCCTGAGTTAGAACAGTTAGTTATAAAATATGCATTGAAAGAAAT
ATACCAAGTATGGGGTAACTTAATTATTGGTGCCGGTATTGCTAATAAAACATTAGAAGT
AGACGGTATTACAGAATAGGTACTACTCAATCAGCTATGTATGGTGGAGCTAGTGCTCAG
ATACTTCAAATAAATGAAGATATAAAGAACTATTAGATGGTTTAAGAGCTACTTTTGGAT
ATAATATGATAGGATTATAAGGAGGGTTAGAAAATGGAAAACCGTATATGATAGGGCTAA
CTCTAACCCTAATGTTATTAATAAGTCAACAACATATACTACTACAACACAAGCAGATGA
ACAAGATAAACCTAAGTATACTACTAGACTAGAGTTTGATACGATTGACATGATTAGGTT
TATTAATGACCAATTATAAAAGTACTATGGGAAGAAGCATATTTCTGTCCTTGTCTTAAT
CTGATACAGGACATCCTAGAGTGGAATTGCCCTAGATGTCATGGTAAAGGTATTGCATAT
CTACCTCCTAAAGAGACGATAATGGCAATACAGTCTCAAAGAAAGGAACTAACCAGTTAG
ATATAGGTATATTAGATACAGGTACTGCAATAGGTACCACTCAATTAGAAAGAGAATTTC
CTATAGAGACAGGTTTACTGTTCCTGAGGTATTGATGCCCCAACAAATGATTTATTTTGT
GAATAAAGATAGAATTAAAAAAGGTATACCTTTATACTACGATGTAAAGAAATAACTTAT
ATAGCCACTCAAGACGGTACAGTCTATGAAGAAGATTATGAAATCAAGAATAATAGATTG
TATTTAAATGAAAATATGAGAATCATACAGTAACTTTAAAGATACTTATGACTTTAAGAT
ATGTAGTATCAGATATACTAAAAGAAAGTCGTTATCAATATACTAAGTTTAATCAACCTA
AATCAAAATTTGAAAACTTACCTCAAAATTACTTCTTAAAAGGGAAGATGTCATTGTACT
ACAAGACCCTTATAAAGTTAATGATGGTATAGAAGAAGACCTAGAAATTCAAGTAGATGA
CCCTAAGGCTTCGGCATCTAATCCTAGTAATTTAGGTGGATTCTTCGGAGGTGCATTTAA
ATAATGCCAGTTCATGGAAAGAGACCTAATTTATTTAAAATAAAACTATAAGCAGGTAGG
TAAGAGAACAATTGATGGTATGCGTTCAGAAGTTCTTGATAAATTACAAGCAACAGCACA
GCAAGTAGAGAATACTAGTATTAAACGTATGCCTACTTATCTACAAATAACAGAGAAAAG
CTTGAAAAGAAGGAGTAGTAGACCTTAAAAAAGCTTTGCTCACTCATCTAAAAAAGAAAA
CTAGTAAAGATGGCGGATGGTATTTAACTGTACCAATCCGCATCAAAACTAGTAGAATGA
ATAACAGTACTTATCAAGATATGAGAACTTTAAAAGTAGATAAAGGAACAGGTTCAGTTT
CGAAGATAACTGATTACCTAGAAGGACGTAGGAAGAATGTAAGCCACCCTTCAATGAAGC
CTGAACCTATGACTCATAATATGACTAAAGTTAAAAGAGGAAAGCAATCTTCTTACTTTA
TATTTAGAACTGTTTCTAGTAAGTCACCTGCTAGTTCTTGGATACTTAACAGAGATAAAG
TTAATGAAGATAACTTCTCTAAAACAACTCTAAAAACTGTTAAGCAATTAATGAACTGGA
AGATGAAAAATTTAAATTAAGAGGAGGGTTAGTATTAAATGGCAATAACATCAGTTGATT
CATATTTATTATCAGAAATAAAGCCTAGACTTAACACTGTGCTAGAAATTGTTATATTAT
AGATGAAGTTTTAAAGACTTTGATTATCAAACTAGAGAGAGCTTTAAAGAAGCTTTCTGG
TAGAAATGCACAACATGAAGTAACGGTAGGATTTAACTTCCCAAAATTTAAAATAACTAT
GAAGCTCATTACTTTGACAGCCAATTAGGTCAAGGACAAGAGACAAAAAACTCTTTAGAA
GGTATTCAGTCATCTTACTTTGAGGCAACAGGAGATACTTTAATGAATCTTCTACAGCAA
TAAGAGAAGATGATAAGTTAGTTTTACTGTTTCTAAACCAATAGGAGAGTTAATAAAGGT
AGAAGATATAGAGTTTGCTAAATACGATAATCTTCAGGTTGAAGGTAATAAGGTATCATT
TAAGTATCAAACAAATGAAGATTATGAGAACTACAATGCTAACATTATATTTACCGAAAA
GAAAAATGATTCTAAAGGTTTAGTAAAAGGATTCACAGTTGAAGAACAAGTAACAGTTGT
AGGTCTTTCATTTAATGTAGACGTTGCAAGATGTTTGGATGCTGTACTGAAAATGATTTT
AATATCTATGAGAGATAGTATAGAAGAGCAACAAACATTCCAATTACAGAATTTGTCTTT
GGTGATATTGCACCAATAATAGAAGATGGTGACTCAATGATTTTTTGGTAGACCAACAAT
TATTAAGTACACAAGTTCTCTAGATTTGGATTATACTATTACACAAGATATTAATAAACT
AACTTTTAAAGAAAGAAAGGATTGGAAGTAGGATGGCTGAAAAAAAGACACCTGAAAATA
ACACTCTAAATTTAATGGTTATGTTCATATAGATACATTCCTTGATACTGCAAAAGCCCT
TTTAATATGAAGGATTCACAAGTAGCAGGATTTAAAGCTTATATGGAAGGTAGTCATTAT
TTGTTTAGTGAGCAAGAATTCTTACCATCATTAGAGAAGTATCTAGGTAGGGAAATTAGA
TATATAATAACATTCAGATAAGGAGAATTAAATATGGCAGTAGAACCATTCCCAAGAAGA
CCTATTACCCGTCCTCATGCATCTATTGAAGTAGATACTTCAGGTATCGGTGGCTCAGCA
GGTTCAAGTGAAAAAAGTATTTTGCTTAATCGGTCAGGCTGAAGGCGGAGAACCAAATAC
AGTTTATGAATTACGTAACTATTCACAAGCTAAACGTTTATTCCGTTCAGGAGAATTACT
TGATGCAATAGAATTAGCATGGGGTTCTAACCCTAACTATACAGCAGGACGTATTTTAGC
TATGCGTATAGAAGATGCTAAACCTGCTTCAGCGGAAATTGGCGGATTAAAATAACATCT
AAAATCTACGGTAATGTTGCTAACAACATTCAAGTAGGATTAGAAAAAGAATACACTAAG
TGATTCATTACGTTTAAGAGTAATATTCAAGATGACCGTTTCAATGAGGTTTATGATAAT
ATCGGTAATATCTTCACAATCAAGTACAAGGAGAAGAAGCTAACGCAACTTTCTCTGTAG
AACATGATGAAGAAACTCAAAAAGCAAGTCGTTTAGTATTAAAAGTTGGAGACCAAGAAG
TTAAGTCATATGATTTAACTGGTGGAGCTTATGACTACACTAATGCTATTATTACAGACA
TTAATCAATTACCTGATTTCGAAGCTAAATTATCACCTTTCGGAGATAAGAACTTAGAAT
CTAGCAAATTAGATAAAATTGAAAATGCAAATATAAAAGATAAAGCTGTATATGTAAAAG
CAGTTTTGGTGACTTAGAAAAACAAACAGCTTACAATGGTATCGTATCTTTCGAGCAACT
TAATGCAGAAGGAGAAGTACCAAGTAATGTAGAGGTTGAAGCAGGAGAAGAATCAGCTAC
AGTAACTGCTACTTCACCTATTAAAACTATTGAACCGTTTGAGTTAACTAAGTTAAAAGG
CGGTACTAATGGTGAACCACCTGCTACATGGGCAGACAAGTTAGATAAATTTGCATGAAG
GCGGATACTACATTGTTCCATTATCATCTAAACAATCAGTTCATGCAGAGGTAGCTTCTT
TGTTAAAGAACGTTCTGATGCAGGAGAACCAATGAGCTATTGTTGGTGGAGGATTCAATG
AATCTAAAGAACAATTGTTCGGTAGACAAGCATCATTATCTAATCCACGAGTATCATTAG
TAGCTAACTCAGGTACTTTTGTTATGGATGATGGACGTAAAACCACGTACCTGCTTACAT
GGTAGCCGTAGCTCTAGGTGGTCTTGCAAGTGGTTTAGAAATCGGTGAATCAATCACATT
CAAACCACTACGTGTAAGTTCATTAGACCAAATCTATGAGTCAATAGACTTAGATGAATT
AAATGAAAATGGTATTATTAGTATAGAGTTTGTTCGTAACCGTACTAATACATTCTTCAG
AATCGTTGACGATGTAACTACATTCAACGATAAATCAGACCCAGTTAAGGCTGAAATGGC
TGTTGGGGAAGCTAATGACTTCTTAGTAAGTGAGCTTAAAGTTCAACTTGAAGACCAGTT
TATTGGTACTCGTACTATTAATACAAGTGCTTCAATCATTAAAGACTTTATCCAATCTTA
CTTGGGTCGTAAGAAACGTGATAATGAAATTAAAGACTTCCCTGCTGAAGACATTACAAG
TTATTGTTGAAGGTAACGAAGCAAGAATTTCAATGACAGTTTACCCAATCAGAAGCTTCA
AGAAAATCTCTGTTAGCTTGGTTTACAAGCAACAGACATTACAACCTAGTCTAGGTGACG
GAGTACCTGGATTAGGTACTCCTATTAATATAATTTGAATACTTTAGGAGAGTGAATACA
GATGGCATCAGAAGCTAAACAAACCGTCCATACTGGTAATACCGTCCTACTTATGATTAA
AGGTAAACCGGTAGGAAGAGCACAATCAGCATCAGGTCAACGTGAATACGGTACAACTGG
TGTATACGAAATCGGTTCTATCATGCCTCAAGAACACGTATACTTACGTTATGAAGGTAC
AATTACAGTAGAACGTTTACGTATGAAAAAAGAAAACTTTGCAGATTTAGGATATGCTTC
ACTTGGTGAAGAAATTTTAAGAAAGACATCATTGATATTTTAGTAGTAGATAACTTAACG
AAACAAGTTATTATCTCATATCATGGTTGCTCTGCAAATAACTACAATGAAACTTGGCAG
ACAAATGAAATTGTAACAGAAGAAATCGAGTTTAGTTACTTAACAGCAAGTGACAAAGCA
CGTACTTAATAGATTAGACCAACTAAAAGTTGGTCTTTTTATTGACACTTTAAAATTTAT
ATGTTATTATAAATATAATAATTTAAACAGGAGATGTACTAGATGGCAAATAAGAGAAAA
CAATAGGAAAAATGAGTAACACAAGAGCAACATGGAATATTAATCCGGTAACTAAAGTTA
AAAAAGATAAAACAAAATATTCTAGAAAAATAAACATAAAGGTCTTGACAATTATAATTA
ACTAAGGTATATTATTAGTATAACAAAAAAAGGAGATGGGTATATGAGTACATTTTGGTC
AGAAAGAAGAACAACTAATAAAGATAGGCAAGTTAAAAAACATTATACTCAAATGAGTAT
GTATGAAAGAAAGAAATGCAGAGTTATTACAAGAGACAATTACTGAAAATAGAATTATTA
ATTTTACACGACATAGTGCAAAAAAGGTTAAAGGTAAACCAACAACAAATATACCTAAAT
TAATAGGTTTTATTTTTTTAAAATAAGTTTGCCTACGAAAATATCATAGAGTACAATAAC
ACAGATTATAATGGTAATATTGAGAGGAGAATTGTTGTTAAACATCCTAAAGTTATAACT
GTAGAAGGAAAACCTAGCTATCAGTTTTGACAATTAGTCTTGAAGATGCTAGAGTTATTA
CGGTGTGGTATAACAGTGTAGATGATACACATAGAACACTAGATTTAAATTATTATAGTA
AAGACTTGACAATTCAATAAGGAGGTATTATAATGGGTATAACAATAGTAAATAGTTATT
TTATTCTGTCTAGCATTTTCCTCATCATATTAACCATATTAAAATGGTAAGGGTACAGTT
ACAAGGAATCATTAACTATGAGTAAAATATTAGTAGTAATAACATCAATTCAATTTTTTA
GCATGTTTAATTATTAATGGTATTTATTGGTCACTAAAATTTATGTAGTAGAACTAGAAT
AAAAGTATTGACAAATTAAAACTAATAAATTATAATAAAGGTATAACAAATTAAAGGAGA
AGATATAAAATGTCACAAGATAAATTAAGAGCAATTTACACAGAAATGAAAGTAGAATTA
CACAAATTTCCTAAAGAGGTAGATATAACAAGTAAATCAACTGCAATTGCAATCAATCAG
ATTTTAGATAAATTCAAAACATTAACAGAACAAGCAGGAAAGATTACTAGAAAATATTTA
GAAGGTCAAGAAATATTAACTATTGATTATGAGTATTATGATTCATTACAAGAATACTAT
ATTTACCTACTTAGAAATAGTGAAAAGATTGAACAAAGTTTACAAGAAATTACTAAGCGT
ACAGGTGAATATGTAAAGTAATTTTGATTTAAAAACAAAATATGATATACTATGTTTAAA
GTAGTAAGCCTACACTAGTCCGTGTTATATTAATATTGAATCGGATAAGCGTAGGCTTTA
TTAATATTTAAAAAGGAAGGTATATCATATTATGGCAGAAAGAAATTAAAAAGGAACAAG
ATGTACAAGAAACAACTAAAGAAGAAAAAAAAGATGTTAGTAAAATGACACCGGAAGAAA
TAGATAAATTAAAATATCAAGACAAACAAGAAAAGAACAAGTTATTAACAAAGTTATTAA
AGGCGTTAATGATACTTGGGAAAAGAATATAACTTTGAAGAACTAGACTTAAGATTTAAA
GTTAAGATTAAATTACCTAATGCACGAGAACAAGGTAATATCTTTGCGTTACGTTCTGCT
TACTTAGGTGGTATGGATATGTACCAAACAGACCAAGTGATTAGAGCATATCAAATGTTA
GCTACCTTACAGGAAGTAGGTATTGAAGTTCCTAAGGAATTCCAAGACCCTGACGATATT
TATAACTTATATCCTTTAACTGTTATGTATGAAGATTGCCAGGATTCTTAAACTCCTTTC
GTTACTAATAGTATAGAAACATTAGATAAAGATATAGAACGATTGGGCGGTATGGAATCA
ATTGTTAAACAACCTTTATCTAGAAATCTATGGGCTATTATGAAAGAGTTTAATGTTTTA
CCTACCGAGCAAAGATTTAAGGACTTAGACGATTATCAGATAGAGTTTATTATTGGGAAT
ATGAATAGAGATGTTTATGAACATAATAAACAACTTAAACAAGCTCAAAAGGTGGAAAAT
TCGATAGTCAATTCGAAGATGATGATAGTAGTTGGTGGAATGAATCTCATGAAGACTTTG
ACCCAGTACCTGATTTCTTAGATGCTGATGATTTAGCACAACAGATGGAAGCTAAATTAT
CCGATAGAGATAAGGAAGAAAGAGCTAAGAGAAACGATGCAGAGTTAAATGATGAAACAG
AAGGACTTACTACACAACATCTAGCTATGATGGAATACATCAGACAGAAACAACAAGAAT
TAGATGATGAAGTAGGAAATGGTAAGACTAGTGAAGATGACGCTACTATATCACAAGATA
GCGTTAATAAAGCACTAGAAGACCTAGATGATGACTGGTATATGTAAAGGGTGGTAGGTG
ATACTACCATCCTTATTTTTTTAAAATGGATGGTGAATAATGATGGCAATGAATGACGAT
TATAGATTGGTCTTGTCCGGTGATAGTTCGGATTTAGAGAATAGTCTAAAGGCAATAGAA
CTTTATATGGATTCTTTAGAGTCTAAGAATATTGATGCTCCTTTAGATAATTTCTTAAAA
AAATTAAAAGTAATTGCTAAAGAAGTTAAAATGTACAGAACGCAATGGATAAACAAGATG
GTAAATCTGTTATATCTTCTAAAGACATGGATGAATCTATTAAATCCACTCAATCTGCTA
CAAAGAATATAAATGAATTAAAGAAAGCTTTAGATGACCTTCAAAAAGAGAATATATCTA
AAGGTATTGCACCTGACCCTGAAGTTGAAAAAAGCATATGCTAAGATGGGTAAAGTTGTA
GATGAAACTCAAGAAAAACTTGAGAAAATGTCTTCACAAAAAATAGGTTCTGATGCCAGT
ATTCAAAATAGAATTAAGGAAATGAAAACCTTAAATCAAGTAACTGAAGAATACAATAAA
ATAAGTAAAGATTCTAGCGCAACTAAAGATTATACAAAACGATTAAGAGCTAATCGTAAT
ATGACTAGAGGTTACATGGAGCGTTCAGAAGGAACAGGACGATTAACATATGACCAAGGT
GCACGAGTTAGAAGTGAGCTAGGTAAAATAAGTTCTTATGAGAGCCAAAGAAAACAAAAC
CAACGTAATTTAGGACAAGCAAGAGAGCAATATAGCAACTATAGAAATCAACAACAAGAC
TTGACTAAACGTAGAGCTAGCGGTCAAATTAATAAGGAACAATATGAACAAGAGTTAGCT
TCTATTAAACAGGAAATGAAAGCTAGAGAAGAACTTATATCTAACTATGAGAAATTAGGA
GCAGAACTTGATAAAACAGTTCAGTATTATAAGGGTTCAGTTCAAAAGGATTTCCAATCT
AGAGACGTAGACCAACAAAGAGGAACATTTGGTAGAATGGTTCAAGAACGTTTGCCATCT
ATTGGTTCTCATGCTATGATGGGTACTACAGCTATGGCTACAGGTTTATACATGAAGGGT
GCCTCATTAAGTGAAACTAATAGACCGATGGTTACATCATTAGGTCAAAATTCCGATAAT
ATGGATATAGATTCTGTAAGAAATGCATATGGAGACTTGTCAATTGATAACAAATTAGGT
TATAATAGTACTGACATGTTGAAAATGGCTACTTCATATGAAGCATCAGTAGGACATAAA
AGTGATGAGGACACAATGGCAGGAACTAAACAGCTTGCTATTGGAGGACGTTCTTTAGGC
ATTAAAGACCAAGAAGCTTATCAAGAGTCTATGGGTCAAATCATGCATACCGGTGGAGTA
AATTCTGATAACATGAAGGAAATGCAAGATGCATTCTTAGGTGGTATTAAACAATCAGGC
ATGGTTGGTCGTCAAGATGAACAACTTAAAGCACTAGGTTCTATAGCGGAACAATCAGGA
GAAGGAAGAACTCTAACTAAAGACCAAATGAGTAACCTTACTGCCATGCAATCTACTTTT
GCAGAGTCAGGAAGTAAAGGATTACAAGGTGAACAAGGTGCCAATGCTATTAACAGTATA
GACCAAGGACTTAAAATGGTATGAATAGTTCTTATGCTCGTATAGCAATGGGATGGGGAA
CGCAATACCAAGGTCTTGAAGGTGGATATGATTTACAAAAAAACGTATGGATGAAGGTAT
ATCTAATCCTGAAAACTTGACAGATATGGCTGATATAGCTACTCAAATGGGTGGCAGTGA
AAAAGAACAAAAATACCTATTTAATAGAAGTATGAAAGAAATAGGCGCTAACCTAACTAT
GGAGCAATCTGATGAAATATTTAAAGATGCTCAATCCGGAAAACTATCTAAAGAAGAGTT
AGCTAAGAAAGCTAAGAAAATGGAAAAGAAGGTAAAAAAGAAGGAGAAGATAACGCCACT
GATTATAAAGAATCTAAATCAGGAAAAAATGACCAAAATAAATCGAAGACTGATGATAAA
GCAGAAGATACTTATGATATGGCTCAACCACTAAGAGATGCTCATAGTGCTTTAGCAGGT
CTTCCTGCCCCTATATATTTAGCTATAGAGCTATAGGAGCATTTACAGCTTCACTAATTG
CATCTGCAAAGTCAATTTGGAGCAGGTCACTTAATTGGTAAAGGAGCCAAAGGACTTAGA
AATAAATTTGGTAGAAATAAAGGCGGTAGCTCCGGTGGTAACCCTATGGCAGGTGGAATG
CCTAGTGGTGGTGGTTCACCTAAGGGTGGAGGCTCACCTAAAGGTGGGGGCACTCGTTCT
ACTGGAGGAAAAATACTTGATAGCGCTAAAGGTCTTGGAGGATTCCTAGTAGGTGGCGCA
GGATGGAAAGGTATGTTTGGCGGGGAGTCTAAAGGTAAAGGCTTTAAACAAACATCTAAA
GAAGCCTGGTCAGGTACTAGAAAAGTATTTAATAGAGATAATGGTAGAAAAGCCATGGAT
AAATCTAAAGATATAGCTAAAGGTACCGGTAGTGGTCTTAAAGATATCTATAATGATAGT
ATATTTGGTAAAGAAAGAAGACAAAACCTAGGAGAAAAAAGCTAAAGGTTTTGGTGGCAA
AGCTAAGGGTCTCTATGGTAAGTTTGCTGATAAGTTTGGTGACGGAGGTAAAAATGGTAT
TCTTTCACAATCACCAAAAGCAGGTGGAAGTGGCATAGGGAAACTTGGAAAACTTGCAGG
TGGACTTGGAAAAGGAGCCGGAGTTTTAGGTGTTGCTACGTCTGCCTTATCATTAATACC
TGCTTTAGCTTCCGGAGATAGTAAAGCTATCGGCGGAGGAATAGGCTCTATGGGTGGAGG
AATGGCAGGTGCATCAGCAGGAGCTTCTATAGGAGCTTTATTTGGTGGTGTAGGTGCAAT
ACCTGGAGCTTTAATAGGTGGAGCTATAGGTTCCTTCGGAGGAGGAGCTGTTGGTGAAAA
AGTCGGAGACATGGCTAAAAAAGCTAACACTAAAGAAGGATGGAACCTAGGATGGACTAA
CGGAGATAAGGATGGTAAGAATAAATTCCAAGATTCTTTATTAGGAAAACCTATATTCAA
AGCATGGAGCGGTATAACAGGTCTCTTTGATAATGACGCTGAAGCATCCGAAGAAGATAG
TAAAGATAAGAAAAAAAGGTGTTAAAGGCGTTAAAGGAGATACTAAGAAGAAAGAAAAAT
GACAGCAGAACAACTTAGAGAAAAGAATAACCAATCTGAAACTAAGAATCTTAAAATCTA
TAGTGATTTACTTGACAGAGCTCAGAAAATTATTGAGAGTGCTAAAGGTATTAATATAGA
TGGAGGAACTTCTGATAGTGGTTCTGATAGTGGAGGCTCTGCATCTGATGTAGGTGGAGA
AGGCGCAGAGAAGATGTACAAGTTCCTTAAAGGAAAAGGACTATCTGATAATCAGGTAGG
AGCTGTTATGGGGAACTTACAACAAGAATCTAATCTTGACCTAATGCTAAGAATGCTTCT
AGTGGAGCATTTGGTATTGCTCAGTGGTTAGGGGCTAGAAAAAACAGGATTAGAAAATTT
TGCTAAATCTAAAGGTAAAAAATCTAGTGACATGGATGTTCAATTAGATTACCTATGGAA
AGAAATGCAGTCTGATTATGAAAGCAATAATCTTAAAATGCAGGTTGGAGCAAAGGTGGA
AGCTTAGAGCAGAATACAAAAGCATTTGCTACTGGATTTGAACGTATGGGAGCAAACGAG
GCTATGATGGGTACTCGTGTTAACAATGCTAAGGAATTCAAGAAGAAATACGGAGGCTCC
GGTGGCGGAGGTGGTGGAGGAGCCCTATCCTCTACTTACCAAGAAGCCATGAGTAATCCT
GTATTAACTACTGGTTCTAATTATAGGGGCTCTAATGATGCTTCTAATGCTTCTACAACT
AACAGAATAACAGTCAATGTTAATGTTCAAGGTGGAAATAATCCTGAAGAAACTGGAGAC
ATTATCGGAGGAAGAATTAGAGAAGTTCTAGATAGTAATATGGATATCTTTGCAAATGAA
CATAAGAGAAGTTATTAGTAATTTTGTATTGACACAAGAGTAGTATCATAATATACTACT
CTTATACATATAAAAAAATAAAAGGAAGTATGTGTATATGCGTAGAATAAGAAGACCTAA
GGTAAGAATAGAAATCGTTACAGATGATAATACATTTACATTGAGATTTGAAGATACACG
TGACTATAATGGTGATGAGTTTGGAGCTAAACTTTTAGGATTCCAAACTAAAACTCTATG
GAAGATGATAGTTCAGTTTTCCAAATAAATATGGCAGGAGATACTTATTGGGATAAGCTA
GTTATGGCTAATGATATCATAAGAATATTTATTACACCTAATGATGACCCTAACGATAAA
GAAGGAAAACAAGAACGACTTATCAGGTAGGTATGGTTTCTCAAGTATCAAAGTAGGTAG
TTACGGTAATGACCAAACTCAATTTAGAATAACAGGTCAATCTTTGTAAAACCTTTTATG
AAATTTGGATTAGGCGTTATTCAGGAAGTTCAAGCTGTATTACCTGAAGTAGGTTGGCTT
ATTGATGGTGATGGAGATAATGAAGTAAAATTTACTGGTAGCTCAGCTCATGAAGTAATG
ACTGGTATTATACGTAGATTTATACCTTATATGAAATATAACTATACTGAAAAACATATA
ATACAATTGATAACTATCTTGATTATGATGATTTAAGTAGTTGGGATGAGTTTGAAAACT
TACAGAAGTTTCAGCCTTTACTAATTTTGATGGGTCATTAAAACAGTTAATGGATATGGT
AACAGCTAGACCTTTTAATGAGTTATTCTTCAAAAAATTCAGAAAAAAACACCTGGAAAG
GCTCAACTTGTATTAAGAAAGACCCCTTTAATCCTACTGAGTGGAGAGCTTTAGATATGA
TTAAAGTACCTACTGAGGATTTTATAGAAGAGGATGTAGGTAAAAGTGATGTAGAGACAT
ATTCTATATTTACAGCAACACCTGCAGGTATGTTGAAAGAGCTTAACGGTGATGTATTTT
CTAAACCACAATTCCACCCTGAATTAACTGATAGATATGGTTATACTAAATTTGAAGTAG
AAAATATTTATCTTAGTACAAAATCAGGTTCAGCTACTGAGGATTCAGATTCTTCAGGTG
ATGATAATGGCACAGAACGAGGAACTTATTCTAAAATTATGAAAGATTTAAGTAACTATG
GAAGAGATAATATATCTAAAAGGTATAGATAAGTATACAAGTAAATTATCTTCAAAATAT
AAAACTTAAAAAAAGCCCAAGCTAAAAAAATTATAGAGAAGTTTGTTAAAGAAGGAAAAG
TAACAGAAAAAGAATATGAAAAAAATAACAGGTAATAAGGTAGATGATGAATTAACATCA
GATAACAGACCGAAGTTGACAAAAGATAAATTAAAGAGTATACTAAAAGAAGTTTAAAAC
ACAAGATGATTTTAATAATTCTAAGAAAAGAAAAGCTAAGACAGATGCACTTAAAGAATT
GACAACTAAATATCGTTTTGGTAATAAAACACATGCTACAACTTTATTAGATGAATATAT
TAAATATAAAGGAGAGCCACCTAACGATGAGGCTTTTGATAAATATCTTAAAGCTATTGA
AGGTGTTAGTAATGTAGCTACAGACACAGGTTCAGATGCAAGTGATAGCCCTTTAGTTAT
GTTTTCTAGAATGCTATTTAATTGGTATCATGGTAACCCTAACTTCTATGCAGGAGATAT
TATTGTTTTAGGAGACCCTAAGTATGACCTAGGTAAAAGATTATTTATTGAAGATAAGCA
ACGAGGAGACACTTGGGAGTTCTATATTGAATCTGTAGAACATAAATTCGATTATAAACA
GGGGTATTATACAACTGTAGGAGTAACTAGAGGTTTAAAAGACGCTATTCTAGAAGATGG
TAAAGGTAGTCCGCATAGATTTGCAGGATTATGGAATCAATCATCAGACTTCATGGGAGG
TCTTATGGGTGAAGATACTTCTAAAGAACTTAAAGAAAAGGTGTAGCAGAGAAACAAAGT
AGTGGAGATAAAGATGGTGGTTCTGATAGTGGTGGAGCTCAAGATGGTGGCTCTTTAGAT
TCACTTAAAAAATATAACGGCAAACTTCCTAAGCATGACCCAAGTTTTGTTCAACCTGGT
AACCGACATTATAAGTATCAGTGTACATGGTATGCTTATAATAGAAGAGGTCAATTAGGC
ATACCTGTGCCTTTATGGGGACGCCGCCGACTGGATAGGTGGTGCTAAAGGAGCAGGTTA
TGGTGTAGGTAGAACACCTAAACAAGGTGCTTGTGTTATATGGCAAAGAAGGAGTTCAGG
AGGTAGCCCACAATATGGTCACGTAGCGTTTGTAGAGAAAGTATTAGATGGAGGTAAAAA
TATTTATCTCTGAACATAACTATGCTACCTAATGGATATGGTACTAGAACGATAGATATG
AGTTCAGCCATAGGTAAGAATGCACAATTCATTTACGATAAGAAATAAAGGAGGATAGTC
TATGGCAACAGATAAAGAAGCTAAAGATGTTATTGATAAATTTATAGACAATGTATTTAA
TTTTGATGTACTTACAAAAGAAAGAATAAAAGAAAAAAGATGAAGAAATTAAAAAAATAA
CTACAGATGATATGTATGAAAAGGTTGTGTATATACGACCTTATGTTGGAGTAATACAAA
GCCTTAACCCTCAGCATGTTCAGTATGAATCATTTTCTAATAATGGTTATGATATAGAGG
CAGAATTAAGTTTCAGGAAAGTAAGTTATTTAGTTGATAAAGGGTCTATACCTACAGATT
CTTTATCTACTTTAACAGTTCATTTAGTAGAACGAAATCAAGAACTATTAATAGATTACT
TTGATGAGATACAAGATGTGTTGTATGGAGAATATATGGAAGAAGAATATGTATTTGATG
AAGATGTACCATTAAGTACGATACTAGCATTAGACTTAAATGATAATCTTAAATCCTTAT
CAAATATAAAGTATATGTTCAAAGGTGCTCCTAAAGAGAATCCATTTGGAACAGATAAAG
ATGTTTATATAGATACTTATAACTTATTATACTGGTTATATTTAGGTGAAGATGAAGAGT
TAGCATATCCTATGAATATTAACTACTTCTTTACAGAGGGAAGATTCTTTACTATATTCG
GTAAAGGACATAAGTATAAGGTAGATGTTAGTAAATTTATAGTTGGAGATATATTATTCT
TTGGTAGAAGTGATACTAATATAGGTATTTATGTAGGAGATGGGGAGTTTATATCTATGA
TGGGTAAATTCCCCTAAAGATGAAACACCTATAGGAAAATATAAACTTGATGATTACTGG
AATGAATTTAACGGAAGAGTTATGAGATTCGATGAAGAGGTGTATATTTAATGGTAGTAA
GATTCCAATCTTCCATGGGGAGAAGTTTAAAAGAGTAGATTCGGATGATTTAAATGTAAA
AGGATTAGTTTTAGCTACAGTTAGTAAAATTAATTATAAATATCAATCAGTAGAAGTTAA
AGTTAACAATTTAACTCTAGGAAGCCGTATAGGTGATGATGGTAGCTTAGCTGTACCTTA
TCCTAAATCTTTCATAGGAAGAACACCTGAAGGAAGCGTATTCGGTACAAAACCTCTTAT
TACTGAAGGTTCTGTAGTATTAATAGGGTTTCTAAATGATGATATAAATAGTCCTATTAT
TTTAAGTGTTTATGGTGATAATGAACAAAATAAAATGATTAATACCAATCCTCTAGATGG
AGGTAAGTTTGATACAGAAAGTGTTTATAAATATAGTAGTTCACTATATGAAATTTTACC
ATCTTTAAATTATAAATATGATGATGGAGAAGGAACAAGTATTAGATCTATAATGGTAAA
TCATTTTCTCTATGACATCAGGTGAAGAAGAGAAACCTCAGGCAACAGATTTTATACTGA
ACTGAGTATCAAGATTTATTTACTTCTTATTATGGTAATAAGACATTAATTGAGCCTAGA
ATACAAAAGGCTCCTAATATGTTATTTAAACATCAAGGAGTTTTTATGATGATGGCACGC
CGGATAATCATATAACTACTTTATTTATATCTGAAAGAGGGATATAAGAGCCTCAGTTTT
AAATACAGAAACACAGAAAAGAACTACACAGGAAATGTCAAGTGATGGGTCTTATAGAGT
TATCAAACAAGATGACGATTTAATGTTGGATGAAGCTCAAGTTTGGATTGAGTATGGTAT
TAGTGAAGATAATAAATTTTATATTAAAAATGACAAGCATAAATTTGAATTTACTGATGA
GGGAATCTATATAGATGATAAACCTATGTTAGAAAACTTAGATGAGAGTATAGCAGAGGC
TATGAAGAATTTGAATGAAATACAAAAAAGAACTCGATGATATAAACTACCTTCGAGGGT
GTAGGTAAAGACAATTTAGAAGAATTAATAGAGTCTACAAAAGAGTCTATAGAAGCTTCT
AAAAAAGCAACTTCAGATGTCAATAGACTTACAACTCAGATAGCAGAAGTGAGTGGTAGA
ACTGAAGGTATTATAACACAGTTCCAAAAAAATTTAGAGATGAGACTTTTAAAGATTTTT
ATGAAGATGCTTCTGTTATTAATGAAGTAAATCAGAATTTCCCTACTATGAAAACAGATG
TTAAGACCTTAAAGACTAAAGTTGATAACCTAGAGAAAACTGAAATACCAAATATTAAAA
CTAGATTAACAGAACTAGAGAACAATAATAACAATGCTGATAAAATAATCTCAGATAGAG
GAGAACATATAGGTGCTATGATACAGTTAGAGTGAAAAATGTCACAGTACCTATGAGAAA
ATATATGCCAATACCATGGAGCAAAGTTACTTATAATAATGCAGAGTTTTGGGATTCCTA
ATAATCCTACTCGATTAGTAGTACCCTAAAAGGAATAACAAAAGTAAGAGTTGCAGGTAA
TGTTTTGTGGGACCCCTAACGCCACAGGACAACGTATGTTGAGAATATTGAAAATGGTAC
TTATAGTATAGGATTACCTTATACAAGAGATGTAGCTATATCTACAGCACCTCAGAATGG
TACTAGTGGAGTTATTCCTGTTAAAGAAGGAGATTACTTTGAGTTTGAAGCTTTCCAAGA
CTCAGAAGGTGACAGACAATTCAGAGCAGACCCTTATACATGGCTTAGTATTGAAGCTAT
AGAATTAGAAACTGAAACTATGGAGAAAGACTTTATGCTTATAGGACATAGAGGAGCAAA
CTGGATACACAGATGAGCACACGATAAAAGGATATCAAATGGCTTTAGATAAAGGTGCAG
ATTATATAGAATTGATTTACAATTAACAAAAGATAATAAGTTATTGTGTATGCATGATTC
TACTATATAGACAGAACAACAACAGGAACAGGTAAGGTAGGAGATATGACCTTATCTTAT
ATACAACCAATCCTTACATCTCTCAATGGTGAGCCGATACCATCTCTTGATGATGTACTA
AATCATTTTGGAACAAAAGTTAAATATTATATAGAAACTAAACGTCCGTTTGATGCTAAT
ATGGATAGAGAATTATTAACTCAATTAAAAGCAAAAGGATTAATAGGAATAGGTTCAGAG
ATTCCAAGTAATTATTCAATCATTTGCTAGAGAATCTTTAATTAATATTCATAATCAATT
CTCTAATATACCTTTAGCTACCTAACAAGTACATTTTCTGAAAGTGAAATGGATGATTGT
TTAAGTTATGGTTTTATGCTATTGCGCCTAAATATACAACTATAACTAAAGAATTAGTAG
ATTTAGCTCATAGTAAAGGGCTTAAAGTCCATGCATGGACGGTAAACACAAAGAAGAAAT
GCAAAGCTTAATACAAATGGGTGTAGATGGATTCTTTACAAACTACCTAGATGAATATAA
AAGATTTAATATTAAAGACCTATTAATTTAGGTCTTTTTTTTTAGTTGTAATTTAAACTA
GTTCGTGATATATTAGTAGTATGAGATTTATATACATGCTGAAAAGGAGAGGATAAAATG
CCACAATCAGATGGAATAAGTAATCTTCATAGAATAGCTTTACGCTTCCCTAAAGAAGGC
GGTGGTTATGATATGTATAGATTTAAAGTTAACCCTGAGAACTACACAATAGATTCACCA
CAACGTACGACAGCAATTAAAACAAAATCAGATATTGTAATAGAAGATTATGGTAAAGAC
ATAGAAGTTATTAACTTCACAGGTACAACTGGTTTTAGACCTGTTAGAGAAGCAGATGGA
TTAAAACAGGTAAGCAGAAAATGGAAGAGTTACAAAGTAGAGTTAGTGAATATGCTATGC
AAGGTGGCAGTGGTAATGTAAGTGGTTCTTACTTACAATTTTTTAACTTTACAGATGATA
GTTATTATAAAGTTCATTTAGCTCCTCAGGGGTTAAAGATAACTAGGTCTAAAGATGAAC
CATTACTTTTTAGATATGAAATAACATTAGTAGTTATTGGTTCATTAACAGAAGCAGATA
GAAGTGCTGTAACAACAGAAGAGTTTGGTAACGTTAAACCTAATGCTTCTCAAAGAGTAG
ATGAGGGTATAAAAGAATTAGATAAAATGCTCGTAAAACGAGAGATAGAAACAATCAAGA
AATATCTAGAAGAGAAAATACAATACCTAAATCTACAGGAGATAATACGAACGAGGGTAA
TAGACTTAAGCAAAGCTTCCCTAGTTCTATATATAATCCTAGACAATCTACTAACGGATT
AAAAGGTAATATTGACAATATGGCGCTGATAATAGGTTACGGTGATGGAGGTGTATCTAG
CTAATGAATAATTTTATACCACAACCTCAAGGTCTACTTAGATTTTAAATGCCCTAGATA
CAGATTTAACTTCTTCTCATATGAATTTACTGGATGAAGAGGTATCATTTGTATCTAAAT
TTTATACACCACAGCTACAATTAAGTGAATTAGCAAAAAAAGTATTGACAAATATAAAGA
CAGATGATATACCTGTATTAGAAAGAGAATTTAATGATAATACAATTATCCATAAAGCTA
ACGATACATTACTAAAGTACAGGCTCCAAGAATGTATATGATTCTACAGTCGATTGTACT
TGAAGCATATGCTATTGTTAATTGCTTTGTAGAAAATCCGAGCTCTTTAAAATACTTAAC
TGAAGAAGATGTTAGTATAACACGGAAAATTTTAAATTATGTAGCTGACTACTTAGGTAA
CTATGATGACTATAATAGTGTTGTCTTAGACTTAAGAGATTTAGACTTATGTTTTAGTGC
TATAGAATTACAATTACCTCTAATCAAAAGGAGGCTAACGTATAATGAGATTTAAGAAGC
ACGTAGTTCAACATGAAGAAACGATGCAAGCAATAGCACAGAGATACTATGGTGATGTGA
GTTATTGATAGACCTAGTAGAGCATAATAATTTAAAGTACCCTATTTAGTAGAAACTGAT
GAAGAAAAAATGAAAGACCCTGAACGATTGGCTTCTACAGGTGATACACTGATTATACCT
ATAGAATCTGATTTAACAGATGTATCAGCAAAGAAATTAATTCTAGAGATAAAGATGTAC
TAGTTGAATTAGCTTTAGGAAGAGATTTAAATATTACTGCAGATGAAAAGTATTTTAATG
AACATGGTACTAGTGATAATATACTAGCATTCAGCACAAATGGTAATGGAGATTTAGATA
CTGTAAAAGGCATAGATAATATGAAACAGCAATTACAGGCACGTTTATTAACTCCTAGAG
GTTCTTTAATGCTACATCCTAATTACGGTTCAGATTTGCATAATTTATTTGGTCTTAATA
TACCTGAACAAGCTACATTAATAGAAATGGAAGTATTGAGAACATTAACATCAGATAATA
GAGTAAAATCTGCTAATCTAATTGATTGGAAAATTCAAGGTAATGTTTATTCAGGTCAAT
TTTCAGTGGAAATAAAATCTGTTGAAGAATCAATAAATTTTGTCTTAGGACAAGATGAGG
AAGGAATTTGCTTTATTTGAATAGGAAAGGATTAAATTATGAAAACTAGAAAATTAACTA
ACATACTATCAAAATTAATAGATAAGACAATGGCAGGTACAAGCAAGATAACAGACTTTA
CTCCTGGTTCAGCTTCTCGTTCATTATTAGAAGCTGTATCATTAGAATAGAGCAATTCTA
TATTCTAACAAAGAAAATATTGATTGGGGTATACAAGAAGGTATCATTGAAGCTTTTGAT
TTTCAAAAAGACAATCTAAAAGAGCTTATGGTGATGTTACTATTCAATTCTACCAACCCT
TAGATATGAGAATGTATATACCCGCAGGAACAACTTTTACTTCAACACGACAAGAATACC
CTCAGCAATTTGAAACATTAGTTGATTATTATGCAGAGCCTGATTCTACTGAGATTGTTG
TTGAAGTTTATTGTAAAGAAACAGGGTTGCAGGTAATGTTCCTGAAGGAACAATTAATAC
TATAGCATCAGGTTCTAGTTTGATTAGAAGTGTTAATAACGAGTATTCTTTTAATACAGG
AACTAAAGAAGAGAGCCAAGAAGACTTTAAGCGCAGATTCCACTCTTTTGTAGAATCTAG
AGGTAGAGCAACTAATAAATCAGTAAGATATGGTGCACTGCAGATACCTGATGTAGAAGG
TGTTTATGTTTATGAAGAAACAGGACATATTACAGTATTATTTGCTCATGATAGAAACGG
TAATTTATCAGATGATACCTTAAAAGAAGATATAATTGATGCTTTACAAGACTATAGACC
AGGAAGTGGTATAATGTTAGATGTTAGGTGTAGGAAAAGAAGTTAATGTTTCTGCTACAG
TAACTATATCTAATAAATCTAGAATTGGTGATACATTACAAAAAACATATCGAAAGTGTT
ATTAGAAGCTATTTAAATAATTTAAAAACTTCTGATGACCTAATAATTACAGACCTTATT
CAAGCTATAATGAATATTGATGATGTATTAATATATGATGTGTCATTTGATAACCTAGAT
GAGAACATTATAGTGCCGCCACAAAGGAATTATTAGAGCAGGAGAAATAAAAGTAGAACT
AAAGTAAAGAGAGGTGAAACTTAAGTCGTGGCTAATTTTTTAAAGAATCTTCATCCATTA
TTAAGAAGAGATAGAAATAAAAAAAGATAATCAAGACCCTAACTTTGCTCTGATAGATGC
ACTCAATGAAGAGATGAATCAAGTAGAGAAAGATGCTATAGAAAGTAAGTTACAATCTTC
TCTAAAGACATCTACCAGTGAATATTTAGATAAGTTTGGGGATTGGTTCGGAGTTTATCG
TAAGACCGATGAGAAAGATGATGTTTATAGAGCAAGAATTATAAAATATTTACTCTTGAA
AAGAGGAACTAATAATGCTATAATAGATGCTATAAAAGATTATTTAGGTAGAGATGATAT
TGATGTAAGTGTATATGAACCTTTTACAAATATTTTCTATACTAACAAATCACATTTAAA
TGGTGAAGACCACTTAATGGGATACTATTATAGATTTGCTGTTATTAATGTCTCTATAGG
TGATTATTTCCCTGTAGAGATTATAGATGTAATTAATGAATTCAAACCTGCAGGTGTAAC
TCTATATGTCACTTATGATGGGGCTTCTACTATTAGAGGTGGAGCAATTATTAAGTGGTT
AGATGGGTTACCTAAAATAGAAACATACCAAGAGTTTGATAGATTTACAGGTTATGATGA
TACATTCTATGGTCATATTAATATGAATCAAAGTAAAGATACTGATAACAGTTCATCAGA
TATTTTTTAAAACAAACCATAGCTTAATTAATAGTTTAGATGTTTTAACAGGTTCATCTA
GTGTAGGGAGACAGTATATTAACTACGGATATGTAACATCATATGTTTATAATCCAGGTA
TGACATCTTCTGTAAATCAAATAAGCGCTAGTACAAAAGGTAGAGGTCAAGAAGTACCTA
CTGACTATTATATGTATACTAGTACTAAGAATAACAATACAGTAGAACTTAGTATGCAAA
CTACTTCCGGTGTGTCTTATTTATATAATAACTTTAATTTTAGGGACTATATGAGTAAAT
ATAGACCTCAAGTAGATTTACAATCTGATGAGGCTAGAAAAGAATTGTATCTGATTATAT
AAAAGAATTAAGTATTGATTACTATCTTAGTGCTGTGATACCTCCCTGATGAAAGTATAG
AAATTAAACTACAAGTTTATGATTTTTCTATTAATAGATGGCTTACAGTATCAATTAATA
ATTTATCTTTCTATGAAAAAAATATCGGGAGCAATATAGGATATATAAAAGATTATCTAA
ACAGTGAATTAAATATGTTTACTAGGTTAGAGATAAATGCAGGTAAAAGAGATTCAGTAG
ATATTAAAGTTAATTACTTAGATTTAATGTTTTATTACTATGAACGAGGTATTTATACAA
TAAAACCGTATAAAGCATTAATAGAAAATTATTTAGATATATCTAGAGAGACTTATGTAG
AAGCATTTAAAATAGCATCATTATCTAATGGAGATATTATAACTAAAACAGGTTTTCAGC
CTATAGGGTATTTAAAACTAGTTGGTAATTATGAAAATACAATACCTAGCACAATAAATA
TAGTAGCTAAAGATACAGATAATAACCCTATAGAATCTAATGAATTAGATGTATATAATA
CAGTAGAGAATAGAAATTTATTACAATCTTATAAAGGTGTAAATACGATAGCTAGAGAAA
TAACTTCTACAAAAGAGTTTACTGTATCAGGATGGGCTAAAGAGATATACTCAACTAATT
ATCTTTCTAAAGTATTAAAACCAGGTAAAGTGTATACGTTATCTTTTGATATGGAAATAA
CAGGTAATGACCCAACTCTTAAATCTTATTCTGATAGTCATGGTATATATTTATACAGTA
ATACTAAGGGAATTGTTGTTAATGGTGTTAAATCTATGGAACGTACTATAGGTAACAAAG
TATCCGTAACTCAAACTTTTACAGCCCCTACTATTACTGACCATAGATTACTAATATATA
CTGGAAGATATACATCTGATGGTAAAGCATCAACTCCTCCAGTGTTCTTTAATACAGTTA
AAATTACGGAATTAAAATTGACTGAGGGTTCTTCTAAGCTAGAGTACTCACCTGCTCCGG
AAGATAAACCTAACGTAATAGAAAAGGAATTAAATTTAATAATATCCTAACTAATATACA
GACTTTAAGTATTAATTCGGATACTATCTTAAAAATGTAACTTTTATATTATTCTTACTA
TGGTGATAGTTGGGTAGAACTAAAGACTCTAGGAAATATTAGTACTGGAGAAACAACAGA
AACCAATAACTTAATAGATTTATATGGATTACAGACAGTAGATTATTCTAATATAAATCC
AATGTCTAAAGTATCATTACGTTCCATTTGGAATGTTAAGCTAGGTGAATTGAACAATCA
AGAAGGTTCTTTATATAATATGCCTAATGATTACTTTAATGCTGTATGGCAGGATATAGA
TAAATTATCAGATATTGAGCTAGGTTCTATGAGAATGGTTAAAGACACTGAGGGCGGAGT
ATTCGATGGAGCTACAGGTGAAATTATTAAGGCTACTCTATTTAATGTCGGTGCTTATAC
TGATTTAGATATGTTATTTATGCTTTTGACTAATTATACTGAACCGTTAACGTTAGGCTC
TAGTCGATTAATAATTGAGCTAGAAGAAGAACTACTAACATCAGAATCATTTAATGTCGA
TAATAGAATTAAAGTAATTGACTCAATATGAGGAGTTACCAAATACAAGCATTATTAAAA
ATAGTTTGTTGAAAAGTTACAGGTTCTAAATATTTAGATTACGGTTTATATGAGCCTATA
GAAGATGGTACTAGATATAAACTTATTGTCGAAGGAGAATTTAAAGATAATATAGAATTT
ATATCTTTATACAATTCTAACCCTAACTTTAATGAAACATTTATATATCCATCAGAGATA
ATTAATGGAGCTGTTGAAAAGAATTTATTGCAAAACCATCTACTGAAGACAAACCAAGGT
TAAATACAGATGTTAGAATATATATACGACCTTATGATTCAACTATCTCTAAAGTAAGAA
GAGAATTAAGGAAAGTTTAATAAATAAGTTGACAGAAAGTTAATAATATGGTATACTTAT
AAAGTAATATTTAGTGGGTATACCATGTTATATTAATAAAGAAAACAACAGATGAAAGGA
ATTAAAAATATGGCAATTGCAACGTATAATTCTCATGTTGAGTTAGCAAATATCTAGTTA
GTGTTGATTCAGTTTACTTAACAATTGAAAGAGCACACCGTGGTCTAATGAAACAAACCC
ACCGCAACCTGATGAAAATGCAACAGTATTACAGGAGGTTATTGAATAAAAAAGCTACTA
AAGTTACTTTAGTTAGACCTCTAAATCACCTGAAGATGATAATAAATTTAATTTCTTATG
GTAATGGCAAATCGTGGGTAGAAGTAACACCTGAAAAATGCTAAAGCTGAAGGCTAAATA
GGTTTTACTTAGAAAGTAGTATTGTTGGTGACGAACTACCTCTTGGAACATATAGACAAG
TAGGATTTGTTATGGACCTCTAGTAGCAAAAGTATTAGTAAATTTAACTTAGTACCTAGT
GAAGTAGAATCAACTGGAACATTATTATTCTTTGATAATAAACAATTCAAAATAGAAGTG
AGCAGACAACTGCTAAAGAAAGATTTATTGTAGAAGTTTAAAGAAAGGGAGATAATTCTA
AATGGCAATTAATTTTAAAGGTTCACCTTTATTTAGATTTGACCCGTCTAAAGATAGAAC
AAAGTATTATTTAATCCTGATAGACCTCTACAACAGGCAGAATTAAATGAAATGCAGTCT
ATAGACCAATATTATTTAAAAAATCTAGGAGACGCTATTTTAAAGACGGAGATAAACAAT
CAGGGCTTGGATTCACATTGTCTGAAGATAATGTATTGACAGTAAATCCTGGTTATGTAT
ATATCAATGGTAAAATAAGATATTACGATAATGACGATTCAGTTAAAATAACTGGCGTAG
GTAAAGAAACTATTGGTATTAAATTAACAGAACGTATTGTTACACCTGATGAAGATGCTA
GCCTATTAGACCAAACTAGTGGAGTACCAAGTTACTTCTCTAAAGGTGCAGATAGATTAG
AAGAAAAGATGTCATTAACAGTTAATGACCCGACATCAACAACTATTTATACTTTCATGG
ATGGGGATTTATATATTCAATCAACTAATGCTGAGATGGATAAAATCAACAAAGTATTAG
CTGAACGTACTTATGATGAGTCAGGTTCATATAAAGTAAATGGTTTTGAACTATTTTCAG
AAGGTAATGCTGAAGATGATGACCACGTTTCTGTAGTTGTAGATGCAGGTAAAGCCTATG
TAAAAGGTTTTAAAGTAGACAAACCCGTATCAACAAGAATTAGTGTACCTAAATCTTATG
ACTTAGGAACAGCAGAAAATGAAAGTACTATCTTTAATAAGTCTAATAACTCTATTATTT
TAGCTAATAGCCCTGTAAAAGAAATTAGACGTGTTACAGGTCAAGTACTTATTGAAAAAA
AAAGAACGAGTTACAAGAGGAGCTCAAGGTGATGGTCAAGATTTTCTTTCAAATAATACA
GCATTTGAAATTGTAAAAGTTTGGACTGAAACAAGCCCTGGAGTTACTACAAAAGAGTAT
AAACAAGGAGAAGACTTCAGATTAACAGATGGTCAAACAATTGATTGGTCACCTCAAGGT
CAAGAACCTTCAGGAGGTACTTCATACTACGTTTCTTATAAATATAACAAACGTATGGAA
GCCGGTAAGGATTATGAAGTAACAACTCAAGGTGAAGGTTAGTAAGAAATGGTACATTAA
CTTTACACCTTCAAATGGTGCTAAACCTATTGACCAAACAGTAGTATTAGTAGACTATAC
TTACTACTTGGCTCGTAAAGATTCAGTGTTTATTAATAAGTATGGTGATATTGCAATATT
ACCTGGTGAACCTAATATTATGAGATTAGTTACACCACCATTAAACACAGACCCTGAGAA
TTTACAATTAGGTACAGTTACAGTATTACCTGATTCAGATGAAGCCGTATGTATTTCATT
TGCAATCACTAGATTGTCTATGGAAGACTTACAGAAAGTTAAAACAAGAGTAGATAACTT
AGAAGGTATAACCAAGCGGTAAATGCTCTAGATGATGGTGCTATGGAAGGACAGAACCCT
CTAACATTACGTTCGGTATTCAGTGAAGGTTTCATTAGTCTTGACAAAGCAGACATTACA
CATCTGACTTCAGGTTGTATTTAGTTTTGAAGATGAAGCTACTCTAGCTTATACAGAAGC
AGTTAACCAACCTAAGATTATTCCAGGAGATACAACAGCTCATATTTGGGGTAGATTAAT
TTCAGCACCATTTACTGAGGAACGTACAATCTACCAAGGTCAAGCATCAGAAACATTAAA
TGTTAACCCTTATAATATTCCTAACAAACAAGGTGTGTTAAAATTAACACCTAGTGAGGA
TAACTGGATTGATACTGAAAATGTTACAATCACTGAACAAAAAACTAAAAAGTAACTATG
AAACGATTTTGGAGACATAATGAAAGTTACTATGGTGAGACTGAGCATTACTTGTATTCT
AACTTACAGTTAGATGCAGGACAAAAGTGGAAAGGTGAAACTTACGCTTATGATAGAGAG
CATGGTCGTACCGGTACTTTATTGGAATCAGGAGGACAACGTACTCTAGAAGAAATGATT
GAATTCATTAGAATCAGAGATGTATCCTTCGAAGTTAAAGGACTAAACCCTAATGATAAT
AATTTATATTTATTATTTGATGGGTAAGATGTGCTATAACACCTGCAACTGGCTATAGAA
AAGGCTCTGAAGATGGTACGATAATGACAGATGCTAAAGGAACAGCTAAAGGTAAGTTTA
CTATTCCTGCAGGTATTCGTTGTGGTAACCGAGAAGTTACACTTAAGAATGCTAACTCTA
CAAGTGCTACAACTTACGGCCAAGGACGTAAAACCGCTCAAGATATTATTATCAGAACTC
GTGTAACAGTAAACAGTAGACCGTTAGCACAATCATTCCAATATGATGAGAATAGAACTA
TATCATCATTAGGATTATACTTTGCTTCTAAAGGTGATAAACAATCTAATGTTGTTATCA
AATTAAGTATGGGTGACCAAGGTTATCTAATAAAACAATCTATGCAGAAACAGTTATGAA
TGCTGATGATATTAAAGTATCTAATAATGCTAGTGCTGAAACTAGAGTATACTTTGATGA
CCTATGATGGCTGAAGGCGGTAAGGAGTACGCTATTGTTATTATTACTGAGAACAGTGAT
TATACAATGTGGGTAGGTACTAGAACTAAGCCTAAAATTGATAAACCTAATGAGGTTATT
TCAGGTAATCCATACCTACAAGGTGTATTATTCAGTTCATCAAACGCATCAACATGGACT
CCTCACCAAAACTCTGACCTTAAATTTGGTATTTATACTTCTAAATTTAATGAGACAGCA
ACGATTGAATTCGAACCAATTAAAGATGTATCAGCGGATAGAATAGTTCTTATGTCTACG
TACTTAACTCCTGAGAGAACAGGATGTACGTGGGAAATGAAATTAATTCTAGATGATATG
GCATCTTCTACAACATTCGACCAATTGAAATGGGAGCCTATCGGTAACTATCAAGACTTA
GATGTTTAGGTCTAGCAAGACAAGTTAAGTTAAGAGCAACTTTCGAATCTAATAGATATA
TCTCACCATTAATGAGCTCTAGTGATTTAACATTCACTACATTCTTAACAGAGTTAACAG
GTTCATATGTTGGTAGAGCTATTGATATGACAGAGGCTCCCTTGTACGGTAAGACTGGGG
TTGCTTGACTTTCTTACCTAAAAGGTACTAAGGATTCCTAAGTATTCTGCGGATGATGGA
AAAACTTGGAAAACATTTACCTAAATCCCCTACAGCTACTAGAGCCAATAATGAGTTTAT
TAATGTTATGTCATTGACGAGAAAGTAAAATCATCAGGAACAAATACTAAACTACAAGTT
AGATTAGATTTATCAACTGAAAATAGCTTTTTTACGTCCTCTCGTGTTCGTAGACTTATG
GTTACTACTAGGGATGAATAAACTAGAGGGTTGATTGACCCCTTTATTTAATAAGGAGAG
ATTTATATGCCTAGAGAAGTTAGAGACCCTTATTCTCAAGCTAAATTATTTATACCTACA
GTTGAGGAAAAAACCAATTAAGGAATTAGAAAACATACAAAGAAAAAAATTGATGAAGCT
ACTAAGTTAATCAATGAATTAAAGAAAGAGAGAGGAGAAAAATAGATGGCATTTAACTAC
ACGCCTCTTACTGAAACACAGAAGTTAAAAGATATGTATCCTAAAGTTAATGATATAGGT
AACTTTTAAAACAGAAGTTAACCTTAGTGATGTAAAACAGATATCACAACCCGACTTTAA
TAATATTTTAGCATCTATACCTGATAGTGGTAACTATTATGTAACTAATTCAAAAGGTGC
TCCTAGTGGAGAAGCTACAGCAGGATTTGTAAGATTGATAAAAGAAATGTAAATTATTAT
AATTTAATATTCACCATAGCAGTAACAAAATGTATATCAAGACTTCTTATGCTAATGGTA
CTGTGGTATATGATTGGATTAGTTTTTTAAATTAGATGACAGAAGCTTATACAATGAAGG
TAATACTTTGAATGTAAAGACACTGAATCCACAACTCAATATGCAACACTGATTAATCCT
CAAAGAACTTAAGCTGGGTTGGGTTAATTACAAAGAAGTAAAAATGGTGTTTCTTCTTTG
ATGAATTTAACCCGGTTAACTCCACTTCAACTTTTAAGATGATAAGAAAGTTACCAGTAC
AAGAACAAAAGCCTAACTTATTGAAAGATAGTTTATTTGTTTATCCTGAAACTAGCTATT
CTAATATTAAAACAGATAACTGGGATACGCCTCCATTTTGGGGATATTCTTCTAATAGTG
GTCGTTCAGGAGTTAGATTTAGAGGAGAGAATACAGTACAGATAGATGATGGGTCTGATA
CGTACCCTTCAGTAGTTTCTAATAGGTTTAAAATGGGTAAAGAACTTTCTGTAGGTGATA
CTGTAACGGTATCAGTATATGCTAAAATTAATGACCCTGTTTTACTTAAAGATAACTTAG
TTTACTTTGAATTAGCAGGATACGATACTGTAGATGATACTAGTAAAAAATCCCATACAG
GAGGACGTAGAGAAATAACAGCAAGTGAGATAACAACTGAGTGGAAAAATACTCTTTCAC
ATTCACTATACCTGAAAATACAATCGGAGCATCAGGCGTTAAAGTTAATTACGTATCTTT
ACTACTAAGAATGAATTGTTCATCTAGTAAAGGTAATGGTGCTGTAGTATACTATGCCTT
ACCTAAATTAGAAAAAATCATCTAAAGTTACACCATTTATTACACATGAAAATGATGTTC
GTAAATATGATGAGATTTGGTCTAATTGGCAAGAATTTATTAGTAAAGATGAATTAAAGG
TCACTCCCCTGTAGATATTGAATATAATGATTATTTTAAATATCAGTGGTGGAAATCTGA
AGTTAATGAAAAGAGTTTAAAAGATTTAGCTATGACAGTACCTCAGGATATCATACATTT
TGTGTCAAGGCTGTGCCGGGACGCCTAAGGGACGTTCTATTAGAGGAACCATTCAGGTAG
ATTATGACAAAGGTGACCCATATAGAGCTAATAAGTTTGTTAAATTATTGTTTACTGACA
CAGAGGGTATTCCTTACACATTATATTATGGTGGTTATAACCAGGGTTGGAAACCCTTAA
AGCAATCAGAAACTTCTACTTTACTATGGAAGGTACTTTAGATTTTGGGTCTACGGAAGC
TGTTAACTTAAATGACTCATTAGATAATTACGATTTAGTGAGGTAACTTATTGGACTCGT
TCAGCAGGACATTTTCTACAAAAGATTAGATATAAAAAATACATCAAGTTTACTGTATAT
TAGAGATTTTAATATTTCAAATGATAGTAAAGGTTCTAGTGTAGACTTTTTTGAAGGGTA
TTGCACTTTTCCTACTAGAACATCAGTACAACCTGGTATGGTAAAATCTATAACTTTAGA
CGGGTCTACAAATACAACAAAAGTAGCATCATGGAATGAAAAGGAACGATATAAGGTATA
CAATATTATGGGAATTAATAGAGGATAAAGAAAGATTGAGTCAAAAAAAACTATGGCTGT
TAAATATGATATAGGTAATAATGAGATAGTATTAATATTTAAGAGAAGGTAAATATATAA
CAGGGTTTACAACAGTAGGAGGGTATGATAAGGAGTTAGGACAAGTAAAAGTTAATAGAA
ATCTTACCTGCTTACTTCTTTGATAATTTTGCCTATGAAAGATATTTGTATTATAGTAAA
CCTGAAGAGGTTATAGAAAATAAAACTATGTACCACCACAAATCAATGATGATGATGAGG
AATCCCAACAAATTACTGTACCTAAAGAACAATATGATAGTTTAAAAGAAGAACTAGAGC
TTATGAGAAAACAACAAGAAGCTATGATGGAAATGCTTCAAAAGCTCTTAGGTCAAAAGG
GGTAATTATAAATGGCGGTAAATTTTACTACAATAACGGAAAAGGCAATGGTATTAGAGA
CCTGACTACTCAGGTCAATAACATTGGAGAAGAATTAACAAAGAAAGAAATATATTTGAC
ATTACCGATGATTTAGTTTATAATTTTAATAAATCACAGAAAATTAAACTAACTGATGAT
AAAGGATTAACTAAATCTTAGAAACATAACAGCCCTTAGAGATATAAAGAACCTGGTTAT
TACTATATAGGTGCTAGAACATTAGCAACATTATTAGATAGATCCCGATATGGAATCTCT
GATGTTGTTTTACATGTAGTACCTCTTGATACTTCTAGTAAGGTAGTTCAACATTTATAT
AATAATTCATCTACTAACAATAACCAAATTAAAATGTTATATAGATTTGTCTCAGGAAAC
TCTAGTTCAGAATGGCAATTTATTCAAGGATTACCTAGTAATAAAAAATGTTGTTATATC
AGGAACTAATATTTTAGATATAGCTTCACCAGGTGTTTACTTTGTTATGGGAATGACAGA
GGAATGCCTAGTGGAGTAAGCTCCGGATTTTTAGACTTAAGTGTAGATAATGATAATAGA
TTAGCTAGACTAACTGATGCTGAAACCGGTAAAGAATATACTAGCATTAAGAAACCTACA
GGAACATACACAGCCTGGAAAAAAAGAATTGAGCCAAAAGATATGGAGAAATATCTACTA
AGTAGTATTAGAGACGATGGTAGTGCATCATTCCCACTCCTAGTTTATACTAGTGATAGT
AAAACATTTCAACAAGCTATTATAGACCATATAGATAGAACAGGTCAAACAACCTTTACT
TTCTATGTTCAAGGCGGTGTATCCGGTTCCCCTATGTCGAATAGTTGTCGAGGGTTATTC
ATGTCAGACACACCTAATACTTCTAGTTTACATGGTGTTTACAATGCTATAGGTACAGAT
GGTAGAAATGTAACAGGTTCAGTGGTAATTGGACTTCACCAAAACATCCCCTTCTCATAA
AGAATTATGGACAGGAGCACAATCATTCTTATCTACAGGAACTACTAATAATTTATCAGA
TGATATTAGTAACTACTCTTATGTAGAAGTTTATACTACACATAAGACAACAGAGAAGAC
TAAAGGTAATGACAATACAGGAACTATATGTCATAAGTTTTATTTAGATGGTAGTGGAAC
TTACGTTTGTTCAGGTAATATTTGTTTCCGGGATAGAACCGATACAAAACCCCTATCACG
GAGTTTTATAGAGTAGGTATCTTTTAAAGGTTCTACATGGACTCTTGTAGATAGTGCAGT
ACAAAATAGTAAAACTCAATACGTTACAAGAATTATAGGTATTAATATGCCATAGACCAG
GAGAAATTTCAGTCTTTTTTTTCTTGACTTGAAAAGGATTCTGTGGTATACTATAACTCG
TGTAAGGATATAAGGAGATTAAAATGAGATTAAGAATTAAGAACTTATATACCTATGTAG
AATTTGAGGAGGATGATAAATACTTAAAAAAGATATTTTAAAGAGAGTCCATACGACTAT
AGGAGCAAGAGAAGAAGGATTTCAGTACAGCCCTGCGTACAAAAGAGGTAGTTGGATGAC
ATGTAGATTTTATGTTTATGAGGAAGATAAATTCCCCACTGGACTTTTATTTAAAATTGA
GTTATTATTAGGTGAGCTACAATCAAGGTATAATTTCCAGTTTGAAACAATTGATGAGCG
TGATGAAAGTTTCTTATCTGGAAGAAGATATTGATGATGAGATAACATTGCTTGATAATA
ATGTCGGTCAAATTACCTTAAGAGATTACCAATATGAAGCAGTGTACAATAGCTTAACAT
TTCCACAACATATTGCTCACTTAGCTACTAATGGTGGTAAAACTGAGGTTGCTAGTGGTA
TTATAGACCAATTATTACCTCAATTGATTAGAAAAAGGTGAGAGAGTAGCATTCTTCACA
GGCTCTACGGAGATATTCCATCAGTCTGCAGATAGGCCTAAGATGTTTAAATATTCCTAT
TGGTAAAGTAGGTGCAGGTAAGTTTGATGTTAAGCAGGTTACAGTTGTAATGATACCTAC
TTTAAATGCAAACAAGACTAACACAAAGGGGTAAAAGGTTACGCCTAAACAAAATATTAG
TAAAAAAAGATTGCTCAAGAGATATTACCTAAATTTGAAGGTGGAACAAATCAAAGAAAT
TACTAAAAGTATTACTTGATAACACAACACCTAAAACAAAAGTAGAACAAAATGTATTAA
GTGCCTTAGAATAATTTACCAAATAGTAAGACAGATGCAGAAGTTTTATTAAACTTAAGA
AATCATAATGCACATTTTCAAAAAATTGTTAGAGAAAGAACGAAAAGAAATATGATAAAT
ATCAAGATATGAGAGATTTTTTTAGACTCAGTTACAGTTATGATAGTTGATGAGGCACAC
CATTCTAAATCTGATTCTTGGTACAATAATTTAATGACATGTGAAAAAAGCTTTATCGAA
TTGCATTAACAGGGTCTATAGATAAAAGATGAATTACTTTGGATGAGATTGCAGGCGCTA
TTCGGTAATGTTATTGCACGAACTACTAATAAGTTTTAATTGATGAAGGTCATTCTGCTA
GACCAACAATAAATATTATACCTGTAGCTAATCCTAATGACATAGATAGAATTGATGATT
ATAGGGAAGCTTACGATAAAGGTATAACAAATAATGATTTTAGGAATAAACTTATTGCAA
AACTAACAGAAAAGTGGTATAATCAAGATAAAGGTACATTGATTATTGTAAACTTCATTG
AACATGGAGACACAATATCAGAAATGTTAAATGATTTAGATGTAGAGCATTACTTCTTAC
ATGGAGAAATAGACTCTGAAACTAGGAGAGAAAAAATTAAACGATATGAGAAGTGGTAAG
CTTAAAGTAATGATAGCTACATCACTTATTGATGAGGGTGTAGATATATCAGGTATTAAT
GCACTAATATTAGGTGCAGGAGGTAAGTCATTAAGACAAACATTGCAACGTATTGGTCGT
GCTTTACGTAAGAAAAAAAGACGATAATACAACACAAATATTTGATTTTAATGATATGAC
AAATAGATTTTTATATACTCATGCTAATGAGCGTGGGAAAGAGGTTGAAGGAAGATTTTG
AAATAAAGACTTAGGAAAATAGGAGGGTAAGAGATGGCAACAAAACACAAAGAAAGCTAT
ACCAATATCTAGAGGAAAATGCTACAGAAAATAAATTTCATATTTCTACTAAGAAAGAGC
TAGCAGATTCTCTAGGTGTTTCCATCTCTGCTTTATCCAATAACCTTAAAAAGTTAGAAG
AAGAAAATAAAGTCGTTACTGTTTCTAAAAGAGGAAAAACGGCGGGTAATAATAACTTTA
GTTAGAGAGTATGACACAGAAGAATTGAAGAATTCAATAATTCTACAGATAATATTATTA
CTTCCGATTTACAGTATGCTAAGGCATTAAGAAGAAAGCACTTCCCTTCTTATAGATATG
AGAGAAAAGAACAACGTAGACGTACTAAGATAGAAATGGCACAATACAATGCCATTAAGG
ATGAGAAGAGAAGAATTATAGCAGATATGAATTTCTATTCAGAAGGTCTTCCTTATCCTT
CTAAAGATATTTTAATATGTCCTATGACCCGAAGGGTTTTATAAAGCGTACATCTTATGT
AAAGTTATACGACCAATATGCTATTTCTCATATGGATGCTAAACATACAAGTCATCTTAA
AGCAATGAGTAAGGCAACAACTAAAGATGAATACGACTACCATCAACATATGTCTGAATA
CCATAGAAAATGATTCAAATTTACCTAGAAATAGCGTTAGTGATAATTTCTTTGGTAGTA
AAATGTTTAATACTTTTATAATTTTTATTTAAAAAATAAAGATAAAAATATTAATGTATT
TAAGTATATGCAAAATGTATTTAAAAATGTAACATTTTATTACGAGAACGGTATGCAACC
TAATCCAATACCTTCTCCTAACTTCTTTAGCTCAGATAAGTATTTTAAAACTATAATAAT
TATATTAAAGGAATAAAAAAAAGGTGTTAACAGTACTAATAGACACCTAGGTGATACAGA
CAGCATCATTAATTCATCAGACTATGTGAAAACCCTGCTGTATTACATCTACACCAACTA
TATACTACAGGATTAAATTCTACTTTACATGATATTGATACTATGTTTGAACAAGCCTTA
GACCTTGAAAATGCCTCCTATGGATTATTTGGAGATATGAAACATATTATTTTACAGTAT
AATTCTATGATTGAAGAAGAAATTAAGAATTTACCTAGAGAAGAAAAGGATATTATTAAT
AAATATGTAAAACAATGCATAATTAATGATTATTCACCAACAAGTATTTCACCTTCTGCA
AGGTTATCAATGTTTACTATGCAGAAAGAGCATATAGTTTACAATAAGCAGTTAAATAAA
GGAATCAAGAGAGGATTTATTACCATTAAGTCTAGGAGGTATAGTGAATAAAGATTTATT
GAGTGGTATGGATATACAAAACTTAGAACAGAATGGTAATGAATACCTATATATGAGACA
ACATACTTCAACTTATTATATATTAAGAATGTTTGGTGACTATTTAGGGTATGAGGTAAA
CTTAAGAGAAGTAAAATATATTGTAGAGAAATATAATTTAATTGATAAAATACCATTGAC
AAAAGAGGGTATGTTGGATTATAATAAACTTATACATTTAGTAGAGGAAGAGGTTAATAA
CTATGAGTAAGAAGATAAAGGAGCTTATCCTTCATAAATCAATGAAGGATATACATTTTG
CAAGAGAAGTATTAGATAACTTACCTAAGAATCTATTTTCAGCAGAGTCTGAGGACATGG
GTTACTTATTTACAGCTATAAAGAGAACAGCACATATTTCCGATAAGATGTCAAATGAAG
CATTAGCAATTAAAGTAGAACAGCTTATGGGTAATAATAAGGAAGATGAAGAGAAGTAAC
CAAGACATTAACTTACTTAGAAGATTTATATAAAGTAGACGTTAATGAAAAAGATGAATC
TGTTAATTATGAAATAGAGAAGTATATTAAAACAGAAATGTCAAAAGAAGTTTTAGTTAA
ATTTATTGCAGAAAATAAACAAGAAGACTCTGATAATCTACATGAACTTGTAGACAAACT
AAAAGCAAATAGAAGTAAGTGACATCTCAGGAGGTAATGGGGAGTTTATTGACTTCTTCG
AAGATACAGAAAAGAAACAAGAACTATTGAGTAATTTAGCTACAAATAAAATTCTCTACT
GGATTTACTTCTATTGACAACCATATTGAAGGTGGTATAGCAAGAGGAGAGGTTGGATTA
ATCATAGCTCCTACCGGTAGAGGTAAATCATTAATGGCTTCAAACTTAGCTAAGAATTAT
GTTAAAAGTGGATTAAGTGTTTTATATATTGCCTTAGAGGAAAAATGGATAGAATGGTTT
TGCGTGCTGAGCAACAAATGGCAGGAGCAGAAAAGAGTCAAATTGTAAATCAGGGATATG
TCTTTAAATAATAAAGTTTATGATGCAATACAAAATCATTATCAGAAGAATAGAAAGTTA
TTAGGTGACTTTTATATTTCTAAACATATGCCAGGTGAAGTTACACCAAACCAATTAGAA
CAAATTATTGTCAATACAACAATTAAGAAGGATAAAAAATATTGATGTTGTTATTATTGA
CTATCCTCACTTAATGAGAAATCCTTATGCTAAATATCATTCAGAATCAGATGCAGGAGG
GAAATTGTTTGAAGATATTCGTAGATTATCACAGCAATATGGATTTGTTTGTTGGACGTT
AGCTCAAACTAACCGTGGTGCTTATGGTTCAGATGTTATTACAAGTGAGCATGTAGAAGG
TTCTCGTAAGATTGTCAATGCTGTTGAGGTGTCTTTAGCAGTAAACCAAAAGATGAAGAA
TTCAAGAGCGGTTTCTTAAGATTGTATTTAGATAAAATTCGTAATAGCTCTAACACAGGA
GAACGATTTGTTAATCTTAAAGTAGAACCAACTAAGATGATTGTAAGAGATGAAACACCT
GAAGAAAACAAGAGCATATACAATTGCTATCAGATAATGGAAAAGAAGACACAAGTAAAT
TTCAAAATAAAGATAATAAAATAGAAGCTATAAATAACACATTCGGAGGATTACCGGGAG
TTTAATTTTTTTAAAATATACCACTTGACATTTTATATGTTAGGTGGTATAATTATTTTA
TAAAGAATAAAGGAGAGATTAATAATGAAATTTGTATTCTTTACAGATAGCCACTTTCAC
TTATTTACTAACTATGCTAAACCTGATGAGCAGTATGTGAATGATAGATTTAGAGAACAG
ATACAAGCTTTACAGAAAATGTTTGATATTGCAAGAGAAGAGGATGCAACAGTTATATTT
GGTGGGGATTTATTCCACAAACGTAACGCAGTAGATACTAGAGTATATAATAAGGTATTT
GAAACATTCCAACTTAATAGAGATATAGAAGTACTAATGTTAAGAGGTAATCATGATCTA
GCGACAAAATAGTTTATATACAGATTCTAGTATAGAACCTTTCGGTTACTTACCTAATGT
AGAGGTTTGTAAAACCTTGATACTTTAGGGTTTTAGGAGAAGAACAGATATTAATATTGT
TATGGCTCCTTATGGAGACGAGACTGAAGAAATTAAAGAGTTTATTAAAAAATAAATATG
TAGAAGATAGAGTAAATATCTTAGTAGGTCATTTAGGTGTAGAAGGCTCTTTGACTGGAA
AAGGGTCTCATAGATTAGAAGGGCATTTGGATACCAGGATTTATTACCTGATAAATATGA
TTTCATTTTACTAGGTCATTATCACCGTAGACAATATTTCCAAAATCCGAATCATTTTAT
GGTGGTTCATTAATGCAACAATCATTTTCTGATGAGCAAGAAGCTAATGGTGTTCATTTA
ATAGATACAGAAAAAAATGACTACAGAATTCATCCCAATCCATACACGTAGATTTATTAC
TATTCAAGGAGAAGATATTCCTGAGAACTTTGAACAGCTAATCGAGGAAGATAATTTTAT
TAGGGTTATCGGTACAGCAAATCATGCTAAGGTTTTAGAAATGGATGACAGTATGAAAGA
TAAGAATGTTGAAGTTCAAATTAAAAGAGTATACTGTAGAGAAACGTATTGATAGTGATG
TGTCTGATGACCCTTTAACAATTGCTAGTACCTATGCTAAACAATACTCACCTGAATCAG
AACAAGAAATACTTGAGTGTTTGAAGGAGGTTTTATAATGAAAAATATAGAGAATATCTA
AATAAGACAGATGCAGAAAATTTAGCAGAGGATTGGGAGAAAGTAACCGAAGATTTATGG
AAAGTGTTTAAAAGATATGAAACCTAAAATTAATACATTAGATATCAGTAATGTAGTAAG
TAAAGACTTAGATAAAAGTAAACCTATTTTACAATTCAAGATTCAGATGGGTAATAGAGA
ATATTTGTAATGTTGAAGGTTTAGAAGATGGTCTAAGTAAAATGAAAAAGATTTTTTGAT
GATAGTAATTTTGAAAACATTATTACAATAGAGTAGTAGACCATGATGAGTATTACTGGA
TTGATTATGGCTCTCATCATTGTTTCTTTAGAGTTACGAAAGGGATAAGTAATGGTTGTA
TTTAAACAAGTAGAAGTTAATAATTTTTTAGCAATTAAAGAAGCTACGCTAGAGTTAGAC
AATAGAGGATTAATTCTAATTGAAGGTGAGAATAAATCTAATGAGTCATTCATTCAAACG
GCTCCAGGAAAATCAACTTTAATATCTGCCATTACTTACGCTTTATATGGTAAAACTGAA
AAAGGACTAAAAGCAGATGATGTAGTAAATAATATTGAGAAGAAAAATACATCTGTTAAA
CTTAAGTTTGATATTGGGGAAGATAGTTATTTAATTGAACGTTATCGTAAAGATAAAGAG
AATAAGAATAAAAGTAAAATTATTCGTTAATGAAAAAAAAAGAGATTACAGGTTCAACAA
ATGACGTTACCGATAAACAAATACAAGATTTATTTGGTATTGAGTTTAATACTTACGTTA
ATGCCATCATGTATGGTCAAGGAGATATCTATGTTCTCTCAAGCAACAGATAAAGGTAGA
AAAGAAATTCTTGAATCTATTACTAAGACAGACGTATATAAACAAGCACAAGATGTAGCA
AAAGAGAAAGTTAAAGAAGTGGGAAGAACAACAAAATAACATAAGACAGGAAATCTATAA
ACTAGGTTATCAGTTATCGACAAAAGATGAGTACTTTCAAAGAGAAATAGAGCAGTACAA
TCAATATAAAGAACAATTGGTTCAGATAGAAAACAGTAATAAGGAAAAAAGATAGATTAA
GAGAACAAGAGGAGAAGCAAATAGAAGCTCAAATAGAGCAACTAGCTTCACAGATACCAA
CAATACCTGAAGATGAATTTAAGCACTCAGAGGAGTATACAAAGCCTCAAAGCCTAGATT
TACTTTCTAATAAATTAACGGAGTTAAATCCAAGTTTACTCAGGTATAATACCAAAGAAC
AAGTACTAAAATCTGAAATAGCTACATTAAGCAATAGTCTAAATCAGTTAGATACAAATG
ACCATTGTCCTGTTTGTTGAAACCCTATAGATAATTCTCATAAATTAAAAGAACAGGAAA
ATATCAATAATCAGATTGAGAATAAGAAACAAGAGATTACTAGTGTATTAGAAATGAAAG
ATACGTATAAAGAAGCTATTGATAAAGTAAAAGATAAATCACAAGAAATTAAAGATAAAA
TGTCACAGGAAGACCAACAAGAACGAGAGCACAATAATAAGATTAACAGCATAATTCAAG
AGGCTTCTAGGATTAAATCAGACATTAGTTCATTAGAGAATAATAAAACGTATTTAAAAG
TTAAATATCAACATCAATCTGTTCAAGGATTAGAGAGAGAAGAACCAAGTAAAGAAAACA
TGAGGAAGATAAGAAAAGAATTACAAGAATCTATTGACAAACATGAAGAGAATATAGTAC
AATTAGAAACTAAGAAAGGTAAATATCAGCAAGCTGTAGATGCTTTTAGTAATAAAGGTA
TACGTTCAGTAGTGTTAGACTTTATTACACCATTCTTAAATGAAAAGCAAATGAGTACCT
TCAAACTTTATCAGGTTCAGATATTGAAATAGAGTTCCAAACTCAAGTGAAGAATGCTAA
AGGAGAACTAAAAGATAAGTTTGATGTTATTGTTAAGAATAGCAAGGGCGGAGGTTCGTA
CAACCAATTCAGCAGGAGAACAAAACGTATTGATTTAGCAATTAGTTTTGCAATTCAGGA
TTTAATTATGAGTAAAGATGAGATATCTACGAATATTGCACTTTACGATGAGTGTTTTGA
TGGATTAGATACTATCGGTTGTGAAAACGTGATTAAATTATTAAAAGATAGACTTAATAC
AGTAGGAACAATATTTGTAATTACTCATAATACCGAGCTTAAACCACTGTTTGAACAAAC
AATTAAAAATCGTAAAAGAAAATGGAGTATCAAAACTGGAGGAAAAATAATGAAATTAAA
GATTTTAGATAAAGATAATGCAACACTTAATGTGTTTCATCGTAATAAGGAGCACAAAAC
AATAGATAATGTACCAACTGCTAACTTAGTTGATTGGTACCCTCTAAGTAATGCTTATGA
GTACAAGTTAAGTAGAAACGGGGAATACTTAGAATTAAAAGATTACGTTCTACTTTACCT
TCATCTTATGGTTTTAGATGATAATAACAAGATATTATTAGAGATAATAACCATAGATGT
AAATAGGTTATTGGTACAACCCTGCAGTACGCAAAGATAATTTAAAGATTATAGAGAAAG
CTAAACAATATGGATTACCTATTATAACAGAAGAATATGATGCTAATACTGTAGAGCAAG
GATTTAGAGATATTGGAGTTATATTCCAAAGTCTTAAAACTATTGTTGTTACTAGATACC
TAGAAGGTAAAACAGAAGAAGAATTAAGAATATTTAACATGAAATCAGAAGAGTCACAAC
TGAATGAAGCACTTAAAGAGAGTGATTTTTCTGTAGATTTAACTTATAGTGACTTAGGAC
AAATTTATAATATGTTGTTATTAATGAAAAATTAGTAAATAGTAAGGAAGGATATTATGA
GGTTTGAAGACTTTTAACCCAAGAATTAGGAGAACCAAAGAAAATACTATAGGTGAGCTA
AGATACTGTTGTCCGTTTTGTGGAGAAAAAAGTTATAAGTTCTATGTTAAGCAAGCCTAG
ACTCTAGTAATGGTCAGTATCATTGTAAAAAATGTGATGAATCAGGTAACCCTATTACAT
TTATGAAGACTTATTATAACATTACAGGTAAACAAGCTTTTGATTTATTAGAGTCTAAGA
ATATAGATATAGAGAGAGCCCCTTACTTACGACCAATAATAAGGATTTGACAGAATCAGA
GAAACTTATATTAATGCTTAGAGGTGTGCACCAAGATAAAGGAAATACTAGTATTAAACC
TCCTAGATTACCTGAAGGATATAAATTATTAAAAGATAACTTAAATAATAAAGAGATTAT
ACCTTTTTTAAAAATGCTTAAAAGGCAGAGGTATAACTTTAGAACAAATTATTAATAATA
ATATAGGTTATGTTATTAATGGGAGCTTTTATAAAGTTGACGGGGAATCCAAAGTATCAT
TATGAATATATTATATTTTTTACTTATGATAATGATGGAAATTACCAGTATTGGAATACA
CGAAGTATAGAAAAGAACCCGTATATTAAATCTATCAATGCCCCTGCTAAACAAGATGAA
GTAGGTAGAAAAGATGTCATATTTAATTTGAATATAGCAAGAAAGAAAAAAGTTCTTAGT
TATAACTGAGGGTGTATTTGATGCTTTAACCTTCCATGAGTATGGCGTAGCAACATTAGG
TAAACAAGTAACTGAGAATCAAATAAAAAAAAATAATTGATTATGTTAGTATAGATACAT
CAATATATATTATGTTAGACACTGATGCATTAGATAATAATATAGACTTAGCTTATAAGT
TAAAACACATTTTAATAAAGTTTACTTTGTACCTCATGGTGATGAAGATGCAAATGATAT
GGGAACAAGGAAAGCTTTTGAGTTATTAAAACAGAACCGGGTGCTAGTAACACCTGAAAG
TATACAGAGTTACAAAAAATACAACAAAACTTAAACTTTAGGCTTGACCTTAGAGAAGTT
TTATGTTATACTAGTAATTAAGTAATTAATAAAGGAGAAAAAAAAATAATGTCAAATAAT
AAAAAAAGATATTTTAGAATTTGTAGATGAATACATTACAGCTTTAAGAGTTGGTAATGA
GCAACGACAACATCAATTAGAAGAAATGGGTAAAGAAGAAACAGCAACATTAACAGATGT
AGCTAAAGCTATTACTAACCTTATGTTAGGTGTTAATGAGCAGATGACAGACTTAGAATA
TAATAACGAGTTAAACTTAAATATTTTTTAATTGATGCTTTATATAAAGCAGAGCTTATT
AATGAAGATGTATTAGACTACATTCAAGAATCAATTGATAAATCACAAGAAGAACCTAAA
ATGAAGAAGAAAAAAGGAGAACAAGAATAATGGAAAAAAATATTAGCACACACACAAAGG
TATTAGTCAAGCAGACATGGAGAAATGGATTGAAGCTGTAGTACAAGGAACTGTTGATGG
TAAACAAGTTGATGAGAAAACAGCTAAACAATTAGATAGAATTGGTTCACGAAGTGTTTC
TTTAGAAGAAGCAACTCGTATTGCTAAAGTTCTTAATGCTGTAACAGCTCAAGAGGTTAC
AGGAGACTTTAATGATGCATTTAATGCAATTGACTTAATGATGATTATCATGGAAGATGA
GTTAGGAGTAACTCAAGAAAAAAGTAGGTAAAGCTAAAGATAAACTAAATGAAAACGAGA
AGCTTACCTAAAAGAGAAACAAGAAGAATTACGTCAAAACAACAAGAAGAGGCACAGAAA
AAACTGAATCTGACAGCAATGAAAAAAGTAATTCAGTTGAAGAAAAATGACGAACAGTAA
GAAAAAGGATACATTCGAACGTAAAATAGCTAAAGAATTAACTGCTTGGTGGGGATACCA
ATTCAATAGGTCTCCTCAATCAGGTGGTGCTTCATGGGGTAAAGATAATAATGCTGTCGG
AGATATAGTACCTCAGGAAGCTAATTTTCCTTTAGTAGTAGAATGTAAACATAGAGAAGA
ATGGACTATAGATAACGTTCTTTTAAACAACAGAGAGCCACACATGGTGGGAGCAAGTCA
TTAATGATAGTAGTAAGGTGAATAAGACAATCTTGCTTAATATTTACTAGAAATAGAGCT
CAGAGTTATGTTGCTTTACCTTATGATGAGAAAGTATATGAAGATTTAAGAAATAATGAA
TACCCTGTCATGAGAACAGACTTTATTATTGATAATATTAGAAAAGATAAATTTTTTTTA
TGATGTCCTTATAACTACCATGAATGGGTTGACCTCATTTACACCTTCTTATATTATATC
TTGCTACGACAAAAGATATAAAACCATACAAGAAGGTCGAGTCTAATTTATCTGAGGTAA
GTAAGCATGAAGATGAATTGATTAATGACCTTCTTAGTGATATATAAGGAAGGTAAGATA
AGTATGACAAGCAAAAGAAAAGACCATTAATCGTATATTTTTTCAGGTACAGGACAAACA
GAAAGATTAGTAAGAAACAAAATTAATATTAATAATTCATTTGAAACATTTAGGGTTAAG
AGTGGAAAAGAAAAAAGTAAATAAACCTTTTATACTAATAACACCTACTTATAAGAAAGG
TGCAATACCTAAACAAATAGAAAGATTCCTAGAAATTAATGGAGCCCTAAAGAAGTTATT
GGCACAGGAAATAAACAATGGGGCTCTAATTTCTGTGGAGCAAGTAAAAAAGATTTCAGA
GATGTTTAAGATTCCTTTAATTGCTAAAGTAGAGCAATCAGGACACTTTAACGAGATACA
ACCAATATTAGAACACTTTAGTAATAAATATAAAGTAGCGTAAAGGATGAGAGATATATG
GCAACATATGGAAAATGGATTGAGTTAAATAATGAAATAACTCAATTAGATGACAATGGA
AAAATAAACTCTATAAAGACCAAGAAGCTTTTAGATGAGTATTTAAAATATATTGAAGAC
AATACAAGAAAGTTTAATAGTGAAGTAGAAAGAATTAGAGTATTGACAAAAGAAGGAACA
TATGATAAAATATTTGACAACGTTCCTGACACTATTATTGATGAAATGACTAAGTTAGCT
TACAGTTTTAATTTTAAATTCCCTAGTTTCATGGCAGGGCAAAAGTTTTATGAATCTTAC
GCATCAAAACAGTATGATGAAAACAAAACCTATTTTTGTTGAAGACTATGAACAACATAA
TGTTCGAGTAGCTTTATATTTATTTCAAAATGACTATGTAAAGGCTAGAGAATTACTAGT
ACAACTTATGGAGCAAACATTCCAACCATCTACACCTACATTATAACAACTCAGGACAAG
CTAATAGAGGTGAACTAAGCTCATGTTATCTATTTGTAGTAGATGATTCAATTGAGTCTT
TAAACTTTGTTGAAGATAGTGTAGCTAATGCTAGTTCTAATGGTGGTGGAGTTGCAATTG
ATTTAACTAGAATTAGACCTAAAGGAGCTCCAGTACGTAATAGACCTAATTCAAGTAAAG
GTGTTATTGCTTTTGCTAAAGCTATTGAACATAAAGTTAGTATTTATGACCAGGGTGGTG
TAAGACAGGGTAGTGGTGCTGTTTACCTAAATATATTCCACAATGATATCTTGGATTTAT
TAAGCTCTAAGAAAATCAATGCCAGTGAGTCTGTTAGACTAGATAAATTATCTATTGGTG
TTACAATCCCTAACAAATTTATGGAGTTAGTTAAAGAAGGTAAACCTTTCTATACTTTTG
ATACTTACGACATTAATAAAGTGTACGGTAAGTATTTAGATGAGCTAAACATTGATGAAT
GGTATGATAAGTTACTAGATAATGATAGTATCGGTAAAGTAAAACATGATGCTAGAGAAG
TTATGACAGATATTGCTAAAACGCAATTAGAATCAGGCTACCTTATGTATTCTATATTGA
TAATGCTAATGATAATCACCATTGAAAACCTAGGTAAAGTTAAAATGAGTAACTTATGTA
CAGAAATTTCACAATTACAAGAGGGTATCAGAAATTTATCCGTACTCTTACAGTAATCAG
AATGTTATTAATAGAGATGTTGTCTGTACATTAGGTTCTCTTAACTTGGTTAATGTGGTT
GAAAAGGTTTATTGAATGAATCTGTAGATATTGGTACAAGAGCATTAACAAAAGTTACTG
ATATTATGGATTTACCTTACTTACCTAGTGTTCAAAAGCAAATGATGATATTAGAGCTAT
CGGTTTAGGTTCAATGAATTTACATGGACTTTTAGCTAAGAATATGATTAGTTATGGTTC
TAGGGAAGCATTAGACCTAGTAAACAGTTTATATAGTGCTATTAACTTCCAGTCTATTAA
GACATCTATGTTAATGGCTAAAGAAACAGGAAAACCATTTAAAGGTTTCGCAGAAGTCCG
ATTATGCTACAGGTGAATACTTTGTAAGATATATTAGAGAATCAATCAACCTAAGACAGA
TAAAGCTAAGAAAGTCTTAAATAAGGTTTATATTCCAACACAAGATGATTGGGATGAATT
AGCTAAAGCAGTAAAAGTACATGGCTTGTATAATGGTTATAGAAAAGCAGAAGCACCTAC
TCAATCTATATCTTATGTACAGAATGCTACAAGTTCTATTATGCCAGTTCCTAGTGCTAT
AGAGAATAGACAATATGGAGATATGGAGACATATTACCCAATGCCTTACCTAAGTCCTAT
AACTCAGTTCTTCTATGAAGGAGAAACAGCTTATAAGATTGACAATAAACGTATTATTAA
TACAAGCGCAGTTGTTCAGAAACATACAGACCAAGCAGTGTCTACAATACTTTATGTAGA
ATCAGAAATCCCTACTAATAAACTAGTATCATTATACTATTATGCTTGGGAACAAGGATT
AAAATCATTATACTATACACGTTCACGTAAACTTTCTGTTATTGAATGTGAAACATGTTC
GGTTTAGAAAGGAAATAGATATGGATATTACACAAAAGTAAAACAACATAATAAAAATGC
TGTATTAAAAGCAACAAACTGGAATATTGAAGATGACGGGATGTCTGATATTTATTGGGA
GCAAGGAATCTCCCAATTTTGGACTCCTGAAGAGTTTGATGTATCAAGAGATTTAAGTTC
TTGGAATAGTTTAACTGAAAGTGAAAAGAACACTTATAAGAAAGTCCTTGCAGGGCTCAC
AGGGCTCGATACAAAGCAAGGAGGAGAAGGTATGAACTTAGTATCCTACCACGAACCAAG
ACCTAAATACCAAGCTGTATTTTGCGTTTATGGGTGGTATGGAAGAGATACATGCTAAAT
CTTATAGTCATATTTTTACAACATTACTAAGTAATAAAGAAACAAGTTATTTATTAGATA
CTTGGGTAGAAGAAAACGACTTTTAAAAGTAAAAGCTCAGTTTATCGGATATTACTACGA
CCAACTATTAAAACCTAATCCTACTATATTTGATAGATACATGGCTAAAGTAGCTAGTGC
CTTTTAGAAAGTGCATTATTCTACTCAGGATTTTATTATCCTTTACTTCTTGCAGGAAGA
GGTCAGATGACACAATCAGGAGCTATTATTTATAAAATTACTCAAGATGAAGCTTACCAT
GGTTCGGCAGTAGGATTAACAGCTCAATATGATTATAATCTTCTAACAGAAGAAGAGAAA
AAACAAGCAGATAAAGAAACTTATGAATTATTAGATATTCTTTACACTAATGAAGTAGCG
TATACACATAGTCTATATGACCCACTAGAATTAAGTGAAGACGTAATTAACTATGTTCAG
TATAATTTTAATAGAGCTCTTCAAAACCTTGGAACGAGAGGACTATTTTAATCCTGAACC
TTATAACCCTATTGTAGAAAATCAAACTAATGTAGACAGATTACGAAATGTTGATTTCTT
TAGTGGTAAAGCAGACTATGAAAAATCTACAAATATCAAAGATATTAAAGATGAAGATTT
CTCATTCTTAGATAGTAAAGAATACAGTACTGCCAAGGAATTCTATAAAAGGAGAAAAGA
TATTATGGATAGAAAAGAAGCAATGGATTTACTAAGTAAAGCAGAAATATTATTTAAAAA
ACATGATGAGTTTTCATGTGTAAGTGATATCAATGACCCTATGAAGTTATTCAGTAACTC
TAAGGATGCTAAAGCTGATGATACGTCTAATTCTTTTCAGCTAGAGTTTATGCATGATAT
GACCATGTATACTTTATCTTATGGCTCAGGACAGCTAAAACTTATTGATTTAGCAGAAGG
TTATGAAGCACAAAAGCTACAATAGTTAACTCATTTCCCGAAATTATTAAAACATTAGAA
AAGGATGATTCAGAAGATGGAAAAAATGAATAGTTTAGTAGATTTAAATACAGCAATTAG
ACAAAAGAAAGATGTTATTGTCATGATTACACAAGATAATTGTGGTAAGTGTGAGATTTT
AAAAAGTGTAATCCCTATGTTTCAAGAGTCAGGTGACATTAAAAAACCTATCTTAACATT
AAATCTAGATGCTGAAGATGTAGATAGAGAAAAAAGCTGTTAAGTTATTCGATATCATGA
GTACACCAGTATTAATTGGGTATAAAGATGGTCAGTTAGTTAAAAGTATGAAGACCAAGT
TACACCTATGCAATTACAAAATTAGAGTCACTTTTAATTTGGAATTTCCTACTATCTGTG
CTATACTATAATAGTACAAGGTAGTAGGATTTTTTAATGGAAGGAAGATGACATATCGCA
AAGAATAAAACATTAACGATATATAATAGTGATAGATATTTTAATATACACACAAAAGAT
AAAGATAAAATTAATGAGGCTATTAAAGTAACACACGGTAATGAAGAAGAAATTGAAAAG
AATATGGATGAATTAATATCTAAGTCTAGAAGATATATCATGAGGGATGAAAAGCATTAC
ATGCTATTTAATGAGAAGTACAATAATGATAGGCTTATAGAAAAGTATGTAAACACGGTG
GTAAGTTACATACTATACTGATTCAGTATTACCTTACTATGTTTTAAAAGACTTATCTAG
TCACCCTGACTCAGAAGTTGTTTATCGTATGCGCAACGGTTTTACTGCAAAAGAAGTAGA
TAATATAGCTTTATCATTCATGGGTACAAAAGTTATTATTGATATTTCTGTAGTATTTCC
TTATGTAAACCCTTATGATATTATTAGAAGTTTACATGATATTAAAACAAATGTAGATGA
AGTTCATTTATCATTTCCACGAATATTAGGGGTAGATGAAAACAAGAAAAGTTTTATTTC
TTTGATGGTGAAGCTTATGATTTAAAACCCGAATATAAAGTCGATTTTGCAGATAAAATT
AGAGTATCTTTATCAGTATGGAAAATGTATATCTATATCTTAACAAGTAGTCGTGATTTT
GAGGATGTAGACAATGTAATTACGAAATTAAAACAACAACGAAAGATTAAGATATAAGGT
GATTATATGAGTACAGCAAATAGAAGAGATATAGCAAGAAAGATATCAGAGAATACAGGT
TACTATATCCAAGATGTAGAGGAAATACTAAGTGCAGAGACAGATGCTATTTCTGACTTG
TTAGAAGAAGGGTATACTAAAGTAAAGAATCATAAATTTATGCAAATAGAAGTTATTGTG
AAAGAAAAGGTAAAAGCGTGGATGTATTCGATCAAAGAATACTTCCATTTACCTAATAGA
AAAGCTATAAAATTCAAACCACTAAAAAGAACTAGAAGAGGTTATTGATAGACTTAATGA
AGAAGAGAAATAATTCTCTTCTTTTTTATTGACAAGGTTTAAAATATATGGTATAGTATT
ATTAAGTTAAAAAAAGGAGAGGAATTAAATGAAAGTATTAATCTTATTTGACCAATATTA
GAGAAGAGCATTTTTCTGTAAGTAAAGATGGGAGTGTGAAATCTAATGTACTAAATACAC
CTAACGGAAAAACACTTAAGAAATTACTTGAGAAGTGTTCTAACTTAAAGAGAGATAAAA
CAAACAGAGATTATGATATTGATTTTCTCTACAATGCAGTACCTACACCTATTAGAAATG
ACTACGGTAAAATCATTAAATACCAAGATGTTAAACAAGCAGAAGTAAAAGCCATACTAA
GGAGAGAATGAATAATATTATTATTGATAATTCTTATGATATGGTAATTCCTGTAGGTAA
ACTAGGTGTTAAATACCTATTAAATGTTACAGCTATTGGTAAAGTAAGAGGTGTACCAAG
TAAAGTAACTATTGAAAATGGAACATCTTCTTCATGATGTGTGGGTATTACCTATAGCAT
TGAATATACTAATGTAAATAAAAATAGTGAACGTCATGTAGTATCAGATTTACAAACAGT
TGGTAAGTTTCAGAGCAAGGAGGATGAACCTAAGGAAGTATCTTACGAGTTGGCAGATAA
CATTGAAAGAGTAAGAGAAATATTCAATAAGGAAGTAAAGAATGATAATTATGATGGGGT
GATATTACCGCATGGGACTTAGAGACTAACCTATTAAAACCTGATAAAGAAGAAGTAAAC
CTTAGTACTATCTCTATCATGGAGAAATGGTCCAAAGTGTAACTATACCCTTATACAAAT
CAGACTTTTAACTAAAACGGTCAAGATGATATTGATGAAGTCTTAGAATTGCTTAAGAAT
TGGTTAGCTAGTAAAGAAGTACATATTAAAGTAGCACATAACGGTAAATGATTTGTTGTT
GTAAAATCCCTCTCATATCGGGCATAGCTTTAAGTAGCTGATAAGAGAACCTAAGTCCTG
TAATAAGGATAGTGGTAATCGAGCTTATTATTGGTGACAATAGATGGGGTGTAGAGACTG
AGCCGAGGTTTTGTAGACCAAGGTGAGACATAGTGTATCGACTTAATAGAGGTGGTACAG
TGAAAAAAAGCATTATATGACATCAGTTAAAAAATAACAAAAGTATGTAGAAGATGCAAC
GAAGAATTAGATTTATCTAACTTTAAAACATATAAGAAGAATGATAAAAACTTATTATCA
AAGTATGTGTATACCTTGTCGGAAGGAATATAATAAGTTAGATAAAGCTAACCACTAAAA
ATGTTATGAGAAAAGGCAGGAGATAAATATAGAAGACAAAGTAATGAGTATAATACTTCT
GACAGAGGTAGAGAGCTAATAAAAATAGGTCTAGGGAAATACAGAGAAAGTAATTCCTGA
AAAGCTAGAAGCCTGTAAGAACCGCATTAAGAAATGGTTCTCTCATAAGACCTGATAAGT
GTTCAGAGTGTAATAAAGATTGCATACCTGAAGCTCACCATCCTGATTATACTAAACCTT
TAGAAATAAAATGGTTATGTAAATCCTGTCATGAAGATACTCATCATAAAAAAATAATCA
CACTATGTAAATGAGGACATCAAGCCCATTTAGGTAACTACAAACAAACCTAATGGTAAG
GGCTTTATGAAGGTATAGTCCGCTCCTATATAGAAATATATAGGCTAAAACGAAATATGA
TATTAAGTTCTTAATGAGTACTGAAAACTTTAAAGATTTTGAGAGTATTCAGGATACTAA
AAGTAGGTTGGTACCTAGCTGTTACTGTTAAGAAGTTAAAGAATCTTTAAGATTATCTGA
TTTAGCTTATGAGGTTACAGATGTCGGAGGCTATGATAAACCATTAGAAGACTTTAAATT
ATGGTTTGTTACTAAGTTATTAAGATTCTTCTCAGATAAAATTAAAGAGATACAGAAAGA
AAATAAAAAGATTGCTAAGAAAGAGTATGATGTTAAAGCTCCTGAATATAAAGAATGGTT
AGAGAATAAATTAAATGAAACAGTAGTAGAACTAGATGATACTGAGAAAAATTTAGAGTT
AGTGAATTAGAGAAAAGTATATTCAACTAGGTCTTTCACCTGAAATTGTAAATATGAATT
TAGTTATGGATAATGATGAATTCATAAATATTGCAGAACAATCACCTGAGTACATGGGGT
TATCTGACTACGCTAAGTCTTACGTTAAATACTGCAATTAATTTAATTAATGAGTATAGA
GATGTAAAAGATGTAGTTAATGATATTGACCGGAGTAACTTTAATTATGATTGGTTCCCT
ATTGAGTTAATGCATCCATACGCATCAGGAGATACTGATGTATGTAGAAGAATTTATTGT
GATGTAATTAAGAAACTTAAAGAACAAGATAGACCTAAGTCAATGCATTTATTAGAAGTT
AATTACCCAAGACTTACTAAGTCTTTAGCTAGAATTGAATCAAATGGTTTATATTGTGAC
TTAGATTATATGAAAGAAAATGATGAGTCATACGAGTCTGAGATGGCTAAGAACCATGCT
ACAATGAGAGGTTAATTGGGCTGTTAAAGAATTTGAAGAATACCAATACAATCTTTACCA
AATGGCGTTAGAAGAACATGAGAAAAAAGCCAAAAGATAGAGATAAAGATATCCATCAGT
ACAGAGATAAATTTAAAGATGGTAAATGGATGTTTTCCCCAAGTTCCGGAGACCATAAAG
GTAGAGTAATTTATGATATTCTAGGAATTCAATTACCTTATGATAAAGAATATGTCAAGG
AAAAACCATTTAATGCTAATGTTAAAGAAGCAGACCTTACTTGGCAGGACTATAAAACAG
ACAAGAAAGCTATTGGTTATGCGTTAGATAATTTAGAATTAAAAGATGATGTTAAAGAGC
TTCTTGAATTACTTAAATATCATGCTAGTATACAGACAAAACGTAATTCATTTACTAAGA
AATTACTTAATATGATTAATAAACAAAACGAACATTACATGGTTCTTTTTTCTGAGACAG
GCACAGAGACATCAAGACTAAGTAGTAGTAACCCTTAAATTGGGGTTGTAAAACTTTGTT
AACTGCGGGAAGAGACTCGTTAGGTCTTAACTACTAACTTATAATGGAAACATATATAAG
GGCAAACAGTAACGTGTTTGATATAGTAAAAGGTTAAGAATAGAGAGAATCCGCATCCAA
GACCCTGAAAGTATATAAAAGTATGGGTAAGGTTCAACGACTAGGTGTTGAGACAATACA
ATCAATACACACCCACGAAAAGCAAAGGTATTATTTCTGTGGTAGGAATAATAAGGAGAG
TTATATGAAAGAGATTTGGAAGAAAGTAGTAGGATTTGAAAACTACGAGGTAAGTAATAA
AGGAAAAGTATGAATATAAAAACTAACTATATTTTAAAGCCGTGGATAATAAATTCCGGA
TATGAGGCAAAGTATCTATAGGTATTGCTAATGTATTAGTACATAGATTAGTGGCTATGA
CATTTATACCTTACTGACAGCTATAGTATAGTTAACCATATTGATAATAATAAATTAAAT
AACTGTGTTGAAAATTTAGAATGGGTGAAGTTACAAGGTAATAGTGCTCACGCTAATAAG
CAAGGAAGATTGAATACTTATAGTGCAAGAGAAAACTTAGTTCTGTATCTAAGAAAGCCA
TTTATCAAAAAAGATATGGAAGGTAACATCATTAAGTTATGGGATTCACCAAGTGAAGCT
GAAAAAGAATCTAATGGGTACTTTAAAAGTACTAAGATTGGTTCCGTTGCTCACGGTAAA
CGTAAGCATCATAGAAGTTATACTTGGGAATACGTATATAAGGATTCAAAGAGAAGTTTA
AATAAGTCTATTAATATGTATGATTTAAATAATAATTTATTATATGAAGATTTGACAATG
AATAAAATTATGGGTATACTAGAAATGAATAATCATAAAACATTAAGAGATAAACTAAGA
AATACAGATGACTTTGTTGAATACAGAGGATATAAATTTAAAATAATAATTAAAACCTAC
CACAGAAATGATATATGATATAGTCTACCAATAGTGAGAGCTATTGTGTTACCTAAACAG
TAACAGATTGTAAACTAAAAGCTTACAAATTATAGAATTTACAAAACTTACCTGCACACA
CATCAGATGTAAATAAGTTTGATTACAAACATCCAATTAAACGTTCATTTGTTTCTAGAT
TTGAAAATGGAGTACTGCTAGGTCCGACTATAGCGCCCTAGAGATGCGTATTATCGGATT
ATTTACTAAAGACCCTGATATGCTACAATCATTCTTAAATGGGGAAGATATCCATAAGGC
TACTGCAAGTATTGTTTACAATAAACCAGTAGAAGAAGTAACTAAAGAAGAACGACAAGC
AACTAAAGCAGTTAACTTCGGATTAGCCTTTGGTGAATCACCCTTCTCATTTGCAGGTAA
AAATAATATGGAAGTAAGTGAAGCAGAAGAAATATTTGAAAAGTATTTCCAAACAAAACC
AAGTGTAAAACTTCTATTGACAATGTACATGAGTTTGTGCAACAATATGGTTATGTTGAT
ACAATGCACGGACATAGAAGATTTATCCGTTCAGCCCAATCAACAGATAAAAGATAAAAA
ATGAAGGTCTAAGACAGTCATTTAACACTATCATCCAAGGTTCAGGTAGTTTCTTAACAA
ACATGTCTTTAACTTACTTAGATGATTTTATTCAATCTCGTAATTTAAAATCAAAGTTAT
TGCCACAGTACATGATAGTATCTTAATTGATTGTCCTCCTGAAGAAGCTAAAATTATGGC
TAAAGTGACAATTCATATTATGGAAAACTTACCATTTGATTTCTTAAAAGCAGAAATTGA
TGGAAAAGAAGTACAATATCTATTGAAGCCGATATGGAAATTGGGTTAAACTATAATGAT
ATGGTTGAATATGATGAGGAAGAAATAGATACATTTAATTCTTACCAAGGTTATATTAAG
TATATGATGAATTTACAGACCTTAGAAGATTATAAAGAGTCAGGTAAACTAACAGATGAA
CAATTTGAAAAGGCTACTAATGTTGTTAAAAGTGAAAACATATTTATCAAGAAATTTAAT
AAAAGTATTGACAATATAGTTAACTTATGTTATACTATATAAGTAATAAATATAAGGAGG
AAAAGAGTGAATACAGGGAGATTAGATTTAATCGTTCTATGGATGAATGGATTATAACAA
GCATGTACCAGGATGAGCTAGGTGGGATGAATATTGTTGTTACATTCTATAATAGAGAAG
AAAATAAACATGGTTCTACAGTTTTACCAACAGAGTCATCTACTGGAGAAGTAACAGAGG
AATTGGCAAGTCTTGAAGAAGAATATCCTTTAGCTTTACCTTTAAGTAGTATCTCAGTTA
ATATTTAAAAGGAGGAACTGATAAATGGAAATACACATTGATTCCCTAGATTTTACAAAC
TTTACTATTAAAGATAGAAATGGGAACTCACAAGAGTTTGATATTACAGATGAGTTAAGA
ATTACAGAGTATACAATACAAGAGGAGTTTTATGCAACAATCAGCTAAATATGCTTTTTT
GGGCTTCTATATTAGAAGGTAAGAGCATATTCTGAAATGGAACAAAGAAACCTAGAAACA
ATTGGTAGTAAGCTAAACCTTACAATTAGACAAGAGTACGAACAACAAGGTAAAAGCCTA
CTAAAGATATGATTGAATCTAGTGTTTATATTCACGATTCTTATCAACAACAACTTAAAG
TTGTTGAGGCTTGAATTATAAAGTTAAACAACTTCAATATGTTGTAAAAGCTTTTGAGAC
AAGAAGAGATATGATGATTCAATTAGGTGCAGAATTACGACAAACAAATAAAAATGGTGG
AATTACTAATCCATTTTCACATTAAAAATAAAGTAAAGAATATAATTGACAAATATAAAA
CTATGTTATAATAAATAAGTAAATTAATTAAAAGGAGAAAAGATAATTATGGATTTCAAT
CAATTTATTAACAATGAGGCAAGCAAATTAGAAAGCAATAACAGTTCTTTTAACAATAAT
GTAGAGAGCTACAAACCTAAAACCCTGTACTACGTTTAGGTAATATTAAAGATGCAAACG
GAAATAAGGTTGTTAAAGAAAATGCTTTTGTACGAGTATTACCTCCTGCACAAGGAACAA
ATGTTTTCTTTAAAGAATTTAGAACAACAGGTATTAACTATTCTAAGAAAGATGGTTCTC
AGGGATTCACAGGGTTAACATTACCTGCAGAAGAGGTTCATCTGTCCTTGACCCATACAT
TCAGGATTGGATAACAAATGGTGTTCAGTTCAGTAGATTCCCTAATAAACCAGGAGTACG
CTATTACATTCATGTTATTGAATACTTTAATAACAATGGTCAAATTCAACCAAAAAACGG
ATGCTCAAGGAAATGTAATGATTCAACCTATGGAATTATCTAATACAGGATATAAAGAAT
TATTAGCTAACTTAAAAGACATATGTTAAAACCATCACCTAATGCACCTCATAGCTTTAT
CTCAGCAACTGAAGCATTCCTAGTTAATATTGTTAAAGCTAAGAAAGGTGAAATGTCATG
GAAAGTAAGTGTTTATCCTAATGCCCCTTTAGGTGCGTTACCTCAAGGTTAGGAACAACA
ATTATCTGACTTAGACCAATTAGCAAAACCAACAGAAGAACAAAATCCTAATTTTGTTAA
CTTCTTAATCAATAACGTTAATAACACAGAGTTAAGTCATGATAACTTTAAATTTAACCG
TGAAACAAATGTCTTAGGTGAAGAACCTTCAGAGCCTAAACAAGCACCCACACAACAAGA
TGTAGATAGTCAAATGCCAAGTAATATGGGAGGACAACCTAATCAGCCTCAGCAAGGTCA
AGTAGGTCAGTATGCACAACAAGGTCAAAGTAATGGTCAAGGACAGCAGTTACAAGGTAC
ACAACAACCTATCAATAACACTCAATTTGGTCAAGGAACTCCTTCAGGACAACAACCAAG
TAACACAGGTTCTGTTGATTGGGATAACTTAGCGCAACAACAATCACAACCTGATTCAAA
CCCATTCAATGATTTTGATGTTAGCAGTGTTGATGATTCACAGGTACCTTTTGAGACACA
ACCTCAAAATACACAACAAGCACCTGAACCACAACAAACTACACAAGAGCCTCCAAAACA
AAACAAACGCAAAGTATTGACGATGTATTAGGTGGTCTAGACTTAGATAACCTATAAGAT
ATAGAGTGCCTTAGAGCACTCTTTTATTTGAATATAATTACTAGGAGGATATTAAATGGC
AAGAGCAAAAAAAGGTAAAGAAGTCGATTTAACAGATTTAAATACAATTGATTTAGGTAA
AGAATTAGGATTAACATTGCTATCAGATACAAACAGAGCAGATATTAAAACGTTATACCT
ACAATGGTTCCTCAGTATGACTATATTTTAGGTGGAGGTATTCCATTAGGTCGGTTAACA
GAAGTTTACGGTTTAACTGGCAGTGGTAAATCTACTTTGCAGTTCACTTATCTCGAATTG
CAACACAATTAGGTGTTATCACTATTTGGATTGATATTGAAGGAACAGCAGATAACAATC
GTATGGAGCAACTTGGTGTAGATGTTTCAAAACTATTCTCTATTCAATCAGGAGAAGGTA
GACTAAAAATACAGTAGAATTATCTGTAGAGCAAGTAGGTAAAGAATTAGAGTACTGGAT
TGACACTTTCAATGAAAGATTCCGGGAGTACCTATTGTATTTATTTGGGACTCATTAGGG
GCTACAAGAACTCAGAAAGAGATTGATGGCGGTATTGATGAGAAGCAAATGGGTCTTAAG
GCATCAGCTACCCAAAAGTAATTAATGCAGTAACACCTAAACTAAATGATACAAACACAG
GGTTAATTGTTATTAACCAAGCCCGTGATGATATGAATGCAGGTATGTATGGTGACCCTA
TTAAATCTACAGGTGGTAGAGCTTTTGAACATAGTGCTAGTTTACGTATTAAGGTTCATA
AAGCATCTCAGTTAAAACAGAAAAGTGGGTTAACTGAGTAAAGATGAATACCATGGTCAC
ATTATGCGTATTGAAACTAAGAAATCTAAACTATCACGACCAGGGCAAAAGCTGAAGCAG
ACTTACTATCTGATTATATGGTAGGTAAAGAAGATGACCCTATCTTATTAAATGGTATCG
ACTTAGAACATACTGTATATAAAGAAGCAGTTGAAAGAGGTTTAATTACCAAAGGAGCAT
GGAGAAACTATGTTACATTGAATGGTGAAGAAATTAAACTTAGAGATGCTGAATGGGTTC
CTGTACTTAAAGATAATAAAGAGTTATATCTAGAATTGTTTAGTAGAGTTTATGGAGAAC
ACTTCCTAATGGTTACTCACCATTACTTAATAACAAAAGTAATCGTAACTCAATTAGAAG
AGTATCAAGCTCTTGAAAACTACTATAAAGAATGGGCTACAGATAATAAACAAGAGGAAC
AAGAGGAAGAACTAAAAGGAGAATCTCAAGAAAAGGATTCTGAATAATAGATGGATAATT
TAATAGATAAAACATGAATCAGGTAAAAGAATCTTTGGGAATGCAAATTCCTCAGATGTT
CTTCCTTTACCTTATAAAGATATAGCAAAGAAATTTGAAGAAGTAAAGAAAAGGTGAATC
AATTATCATTGAAGAAGGTGGATTCCCTTATACAGATTCTACAGTGATGTATATAGAACA
TGTAACAGATAGATGGGCAGGAGGATATTCCTTAATTAGACATGAAGGTGAAGAAGTTAA
AGTACCTAAGACTATCCATTTCTCCTGATATATATGTTAAAGATAAATCACACAAAGTAA
GAATAATCTTCGAGGGGCTAATCCTTATGAAGAAAGCTAATAATGGTAATAGATATGTAA
TAGATATAGATGGTATACCTGTTGATTTTGAAAGGGATTTAGATAGTTTACTTAATAGGT
ATAAAACCTTAGATGGTCGTTATATCATAGGTACGCAGGATTTTATCTAATGATTTTGAA
AGACAAGAACTAAGAGAATATATTGATGAGCAATTTATTAAATTAGTTAAAGAATATAAT
ATTAGAAGTAAAGTGGATTTTCCTGGATATATTAAAGCTAAACTAACTTTAAGAGTTCAA
AATAGTTATGTTAAGAAGAATGAAAAATATAAACGTACTGAAATTATCGGTAAAAAAAGA
TTATACAGTAGAGTCTTTAACAGAAGATTTAAATGAAGACTTCGAGGATAATCAAATTAT
GAGTTATGTATTTGATGATATAGAATTTACAGAGGTTCAAAGTGAGTTACTTAAAGAATT
ACTTATTAACCCTGAAAGAGAAGATGATGCCTTTATCGTTTCTCAAGTAGCGGAAAAGTT
TGATATGAAAAGAAAAGAAGTAGCAAGTGAGTTGACAGAACTCAGAGACTATGTTAGATT
TAAAATAAATGCATACCATGAGTACTATGCTAAGAAAGAATTAAATAACCATAGAGTTAA
TACTGAAAATCATATTTGGGAAAACTAGTTACAGTGCCTTCCTTGTGTTATATTATTATC
GAGAATTCAATAATAAAGCATAGGGAAGGCTTTTTTCTATGTCTTATAGAATGCTTTAAA
ATAGATTACTAAAATAAAGATTGGAGATTAAGCTTATGGCTAAAAGAATGTTAATGATGT
ATTACAACAAGAATCTGTTACAGTAGCAGATAAGTATTTACAAGTTAAAGTTAACCGTGA
CGGTTATACTCGTACACATGAAGGACAATATGCGTACAAAGTAGTTTCAGAGGGAGAATT
ATTCTTATACCCTGTACAAACAGATGGTAAAGGTACATTAAATGTAATGAAGAAATCACC
TATTGCTTACACTGATGGAGACAATATCCATTTCGTAGTAAACACAGTAGTAGACCCTTA
TAATCACTCATTTATCCGTACTGAAGATATTAAAGGATTAGATAAAGGTAAACAACTTAT
TCAAGCTTTCTTAGCTTTCGTTGAAGACCGTTTCAAATTTGGTGTTTATAACGTATTTGT
TGCAAACAACAAAGAGGATGTATTATCTATTGTAGACCCTACAGATAATGATGCAGATGA
AGTTAAAGATAGTTTAGAGCACGCACATGAAGATGTAATTGCGGATTTCCCTGCTAGCCC
TGCTCGTAAGGACGTTAAAGGCGTAGATTCAGGAGAAGGTCAAGGAGACACTTCAGAACC
ATCAGCACCTAAGAACGTTCAAGTTACTCCTAAGGAAGACGGAGCAGACGTATCAGCAGA
ATAATATATAGATAAGGATGGTAAATTGGCTAAGTTAAATTTATACAAAGGTAATGAGTT
ACTAAACAGCGTAGAAAAAACAGAAGGAAAATCAACAATCACGATTGAGAATTTAGATGC
TAATACGGATTACCCTAAAAGGTACTTTTAAAGTATCATTCTCAAATGATTCAGGAGAGT
CAGAGAAGGTCGATGTTCCTCAGTTTAAGACAAAAGCAATTAAAGTTATTTCAGTTACCC
TTGACGTTGATAGTTTAGACCTTACAGTTGGAGATACTCACCAACTATCAACAACTATCA
CGCCTAGTGAAGCATCTAACAAAAATGTGTCATTTGAATCAGACAAATCAGGTGTTGCTA
GCGTAACATCAGAAGGCTTAATTGAAGCAGTTAGTGCAGGAACAGCTAATGTTACTGTAA
CTACTGAAGATGGTAGTCACACTGATATTGTTGTTGTAACAGTTAAGGAACCTATTCCTG
AAGCACCTGCAGACGTAACAGTTGAACCTGGTGAAAATAGCGCAGATATTACTGTATAGG
AGGACAATAAAGAATGGAAAAGACATTAAAAGTTTATAGTAATGGTGAAGTTGTGGGCTC
TCAAGTAGCTAATAACGATGGAGCTACTACAGTATCTATTACAGGCTTAGAAGCCGGAAA
AACTTATGCTAAAGGAGATTTTAAAGTAGCATTTGCTAATGATTCAGGTGAATCAGAAAA
GTAGATGTTCCTGAATTTACAACTAAAACTCCTACTGAAGAACCTTCAGGAGACGCATAA
TAATTAAGACCAACTAAAAAGTTGGTCTTTTTTTTATTGACAATTTATAATATCTATGAT
ACACTATATAAGAATTAAGAAAAGGAGGAAAGTAATGGATATTCCAACAATATTATTTAG
AAATCCATATGATTATACGAAAGTAAAAAAATTAATGGAAAACAAAGAGCAGTATATTGT
AGTAAAGTTTGATTCTGTTTCTGTTCATAATTTAAATGTTCAAGGTATGATGAATGTCAT
CCAAGATTACCTACACATCTATGGTTACAGAGTTAAAGAGTACGGACAAGAAAATTCTTC
TAAAGATGATGAAAGAGACGTTAAAGGCTACTTATATGAAAGAGTAGGTGAGTAGGGTAT
GGGAATTATAGTAAACTCCAACCATATTCAATCAGACACTTTATATGAGTATGATAGCTT
TTTTTGATATTGAGAAAGTAGATACATTTGAAGAAGGATTGCTTTCAATACAGGATGAGC
CAACTGTTTTAGCAGGATTCATCTATGATGATATCACATTTAATAAGGTCATTAATTCTA
ATTCAGATATTGATGATTATATTAAGAATAATGATAATAATTATTTTTTCTGAGAAAATA
AGCCAAATACTAAAAACAGATAAAGCATAGATAGCTGATAGATATACTATATTAGAGTTA
CCTTACTTTTATCTTTTCTATAGATAGAATAACCTAAAGACGTTGTAACACCACTAAGTA
TAAAATAATAGAAACAAAAAAAATTATAGACAGAAAAAAGATACGATAATCATTGTTAAA
CACCTATTTCTTTTGACCTATTATTTCTAGAACTTTTAGATTACACCACTAATATAACAT
TAAAAGCCAGTCATAAAAGTCAATTGTTAGATTAATAATATAATAAAAAAAAGACAATAG
GAGGTTAAAGTGGTTGAATAATAACATAGCTATATTCATATTCAAAACACTGGTTATCAT
TATATTCTTACTACTAATTTTTGTCTGTTATTAATTCCTTGTCCTTATTTACTCAATAAG
ACCGAGTGTGGTTATGACATACTTTATCTTTGGTGGTATTGTTTCTAATGTCACTTACTG
TAACAGATAAGTTCTTACTGAAGAAAGAAGGCCTACCTGAATATGTTCTTAAAAGTAGAG
ATAAATGATAACAAAATAAGAAATAAATTCCAAGAAAAATAATAGAAAGCAATTATGGCA
TAACAAAGCAGAAGAGATAAAAGAAATAAAAAAAGCTACAAAGAAGAGTAGAGGAAGCAT
AGTAAAGGAAGATTACAATGAAAACAAAGAAAGCAAATTAAAGAACAAAGGAAAGAAACT
TAAGGATGGTGCTACATCTGTTTCTTTAGTAAAAAAGGGAGATAAGAGAATACAACTCAG
TAGTAGAATCTTGTAGTCTATGTGGTCAGCAGTTATCAGGTATGAAATTAATAATTAAAA
GGAAAAGCATCAAAAAAAGTTAATCATTTTCATTTACAGTATTCTAAGATATTATTTATT
TTGATATTTGCGCAGATATCAACAATTGTTATAAAATTCTAAGAAAACGAGGTGAAATGG
ATTGAGGCAGGAAAATATTAGAGATATAATTAACAAGAAAAAGTTAGAAGAAGAGGATAC
AAGAAAATATATAGCTGATGGGTTTTATGAATGGTATCGGTAAATTAATGTAATGAATTC
AATAAGAAAGTAGATCAAAAGAAATAGAAGTTAAAAGACCCGAATGAGTACAAATTATTT
GTGATATTCTCTCAAATGCAAAATATGGTCAATGAAACTTCTGAAGGAGAACATGCCTCA
ACTATCTAGACCTCAACAGGAATTATTTGATGAGATTACAACAGAAGATAGTAATGGAGA
ATCTACAGTTGATTTACAGAAGACCTTATCTAGAAATGTCAGCGGAAGATATTGCAGCAA
TGAAATTTCTGAAAGGAAAAAGTAATGAATGAGGAAAAATTCGAAGAAACATTCTAAGGA
GAAAGATATAAATGGATGGAAAAAGAACTAGTAAGATAGCACAAGAAACATTTCAAACTG
AAAAATAACAAGAGAACAGATAGACCATATAATCAATATGCTAAATCCTTTCACCATATG
CTTAAGTATCATACACTGAGAGGGCATCCTATAACTTTTAGTATTCCTAATAGAGATGAA
GTAAGCACAGGCTCATAGACCTTGGCAAACTAGGATTGTAAATGATACTCATCCTAATAA
GGCTGTAATAAAATCACGTCAGTTAGGTCTTAGTGAAATGGGTGTAATGGAAATGGTTCA
TTTTGCAGATATGCATAGTTATGCTAACGCAAGTGTCTTACCCTGCTACAAATGAACAAA
TGAAAAATTTGTTCAGTCACGTTTGAACCCTGTTTTAGAGAAAGAATATTTTAGAGACAT
TGTTGATTGGGATAAAGACTCGTTAGGTTTTAAAAGATAAGAAACTCTAGTTTATTCTTT
AGAACAAGTTCTAAAGCAAGTACCGTAGAGGGTGTGGATATTGATTATTTATCTTTTAGA
TGAGTATGACAGGGTAAACTTATTAGCAGAATCGTCTGCATTAGAATCAATGTCTTCATC
ACCTTTTAAGATTGTGAGAAGATGGAGCACACCTTCTGTACCTGGGATGGGTATACACAA
ATTATACCAACAATCAGACCAGTGGTATTACGGTCATAGATGTCAACATTGTGATTACTT
AAATGAAATGAGTTATAATGATTACAACCCTGATAATCTTGAAGAAAGTGGAAATATGTT
ATGTGTTAATCCTGAAGGTGTAGATGAGCAAGCTAAAACAGTACAAAATGGCAGTTACCA
ATTTGTTTGTCAAAATGTGGTAAACCACTAGATAGATGGTATAACGGTGAGTGGCATTGT
AAGTACCCTGAGCGTACAAAAGGTAATAAAGAATTTACGGTATCTAATAACACAAATGAA
CGCTGTATGGATTTCTGCTGATGAATTAAAAGAGAAAGAAATGAATACAGAATCTAAGCA
AGCATTCTACAACTATATTTTAGGTTATCCTTTTGAAGATGTTAAACTTAGAGTTAATGA
AGAAGATGTTTATGGTAACAAATCACCTATTGCAGAAACACAATTAATGAAACGAGATAG
ATATTCTCATATAGCTATTGGTATAGATTGGGGAAATACTCACTGGATAACTGTTCATGG
TATGTTACCTAATGGTAAGGTAGACTTAATACGATTATTCTCTGTTAAAAAAATGACAAG
ACCTGATTTAGTTGAAGCAGATTTAGAAAAATAATTTGGGAAATATCTAAGTACGACCCT
GATATTATAATTGCAGATAACGGGGACTCAGGTAATAATGTTTTAAAACTCATTAATCAT
TTTGGAAAAGATAAAGTATTTGGATGTACTTATAAATCTTCTCCTAAATCTACCGGACAA
TTAAGACCTGAATTTAATGAGAACAATAACAGGGTTACAGTGGATAAATTAATGCAGAAT
AAAAGATACGTACAAGCACTTAAGACAAAGGATATAAGTGTTTATAGTACAGTAGATGAT
GATTTAAAACTTTCTTAAAACATTGGCAAAATGTTGTTATTATGGATGAAGAAGATGAAA
AAACTGGAGAAATGTACCAAGTTATCAAACGTAAAGGTGACGACCACTATGCACAAGCAA
GTGTCTACGCCTATATAGGATTAACAAGAATAAAGAACTTCTTAAAGAAGGAAACGGTAC
AAGCTTTGGTTCTACATTTGTTTCTACTGATTACAATCAAGAAGGAAATAAACAATTCTA
CTTTGATGAATAGAGGTGAAATAGACTTGACAGATAAATTATTTTATGGTACAATTAGTA
ATGAAGAAATTAATAAAAGTGTATTGAATTTGTTATTGGGTGAGGAATTATCCTTAGATT
ATGTTTCTAAAAATAGTGATATTTTAGATGTTAAATATGAACATGTTTATAAATCTCTAG
GATTCGATAATTTCTTTGATTGTTTTATATGCTAATAGAGAGCCTGAAATAGTCCATAAG
GTGGAGATAAAAATCTTGGTGGACTAAATAAGGTTAAACGTACTGTTATTCGTAATGGTA
AAGAAATGGAAATGACAGTTTACGAAGATGGTAATAAAGAGAACGATAGTAAAGAAAACA
AGAAGGAAAAGAAGAAGTTAGTAGAAGTGCAGTAGGAGCAAGAGCTATTTCTAATGGTGA
AGAAGGAAAGGTAAACCCTAAAAAAGTAGCAAATTCATTATCTAATTTAAGTAAAAAAGG
TGTAGATGTATCCCATATTAATACAAACTCATCATTGTATAAAGAGTTTGTTGATGATAA
CTGTTGATACAATAGGAATTACATCTTTTAAACGAACTGAAAATGATATAATATTAGAAT
CTTATGCAAGTTCACCTGATTCAGATGGTGTAGGAGCAAGAGCTATTATGGAATTATTAC
GTTTAAGTATTAAAGAAAATAAAATGCAGTTGTGTATGACATAGAATTACCTGAAGCAAT
AGAGTATTTAAAACTTTAGGATTTAAACCTAATAAAGATGGATACATCTTAAGAAAAAAG
ATGTAAACAATTCTTAGGTGATTATAGTGATTTTATTTAGCACTATAGTCATCTATTCTA
TTGTATTTATTCTATATATTGTATTAAAACAATTTATATAAAGTCTAATATGAGTAGAAT
AGATAACACAACTGAATTATTAAAATATTACAGGAAGATATTGAAGGTAAGATAAAAAAA
AAGGAAGGAAGAAATAAATGACTTTAGAAGAAAATAAATTAACATTAGAAGAATCAATAA
CTCCACTTAGCAAAGAGGAGAAAGAAGATAGTATTAAAGAATTTAGCAGTTTATTATGTG
AAATGGTAAATAGACTATATAAGTCTTATAATGTATTTAGACAAGACCCTATGGATGAAA
CTCAACGTCTAGATGGCTCTTTAATGGTCTTTCAAAGTAGATTAAATGACCCTTTAACAG
GAGATTTACATGATAAGATGTATAAACTTGCTTTCAAAACGTATTGATATTTTCGAAGCT
AATAAGCAATTTAGAAAAAGATGTAGAAGCAGGTAAAGCAATTGAGTTAGGTGATGTAGC
TATTATAGATACAGCATTAAGTAACATCCTTTCAGGCAATGAGTTCCAAGGAAGTATTTC
ATTTATGCTTAGAAAAGACTTTGAAGAAAAGAACGAATTAGAAAAGAAGAAGAAGAGAAA
CTTAATAACTTATAAAGGGAAGAATTATGAGACTATATAAAATGAGGTATCATAATTGAA
AAAGAAACCACAAGGTAATGAGGTAATCATAACCATAATAACGGTTATGATAGCAGTATT
TGTAGTCATTATGACCATATTTTTAATAAATATCAAGATGCTAAAGAAGATAAAGATAGA
TATCAAAGATTAGTAGAGATTTATAAAAAAGCAGATGATAATGATGGTGAGACTAAAAGA
AATATGTTAAAAGATTAAATAAGGCTGAAGAAGAACTTAAAAAGTAAAAGAAACAAATTA
TAAAGATTATAATAAGAAGTCAAGTAAAGAAAGACAAAAAGAAGATAAAGAAACTAGAGA
GAAAATATATGATGTAACTGGTGATGATGACTTAATATTAGTAAAAAATAATATTGAGTT
TAGTGATAAAGTAGACAAGCCCGAAATACTTATTAGTGAAGATGGAATTGGTACGATAAC
TGTTCCTGTAGATAGTGGGTATGAAAACAAACAGTAGGTTCTATTATTACTAGTGTATTA
GGTTCTCCTTTCCTATCACCTGGTTCAAATAGTATAGATGGTTTAAGTGTTATTAACGAT
AATGTTTATCCAAATACAGTAGATAGCATAGTAGAAGATACAAAACCTTCTATTAACTTA
CCAACGGATAATCCTATTATAACAAATCCAGTTGAACCAACTATACCTTCAGATATTATA
CCTCCTATTGATAATCCTTCAGTTCCGATATCTCCTGAGAACCCAGGAGATAATAATCAA
GGAAATACAGATAATCCAAATCCTCCTCCAGGGTACACAGATGAAGATGGTGGAAGAGGC
TCCGGTGGTGGAGGAAATTCTGAACCACCATCAACGGAAGAACCTTCGGATAATGGTAAC
ACCGGAGGAGGAGATTGGGAAGAAAAACCTGACCCAGGAGAAGAACCTTCAGATAATGGT
AATACAGGAGGAAATGGTGGAGAAGTTACGCCTGAACCTGAACCTGAACCT
SEQ ID NO: 12
TTGTCATAATAATCAAATCCTTTTCTGTTTGTTTTGTTATCTTAATAATATCATGGTTTC
AAGTGACTGTCAATAGTTTTTTTTAAACTTTTTTTATTTAGGTTGGAAGCGGTTGACCGC
TTCCCTTACCTTGTATCTTAATATTAACATCTATTGTTTAAGTTGTCAAGCCTTATTTTG
AATATTTTACAATTTTATTTGCTTTCAAGAAAGTAACATCTTGACGGTAGTTTTTTAACT
CTTCTGTAAACCTTTCATTATTTTTCATGCCGATTAAGTTTAAAGTTGTTGTGATTTTTT
TCATTTTATCATTTCCTTTATTGATTTATTTGTTATCTTAATATTAACATGTTGTTTCTA
GGTTGTCAATAGTTATCTTAAAAAAAATATTTATATTATCTTGTAGTAGTAAAACCAGTT
TACAAGTTTAGGTGTGACTGTTGCAACTAAACCCCTATAATAAGTAAGAGAATGTTATCT
GTTTTTGTGTTGATTTGATTGTTTTGTTTTTCCATTTAAATTTTATCATTTTGTTCAATC
CCTTTCATTTGGTATACCTTTATATTAAACTATAATGATATTAAAGTCAATAGATTTAAT
GAAATTAATTTAAAAAACTTTTACATTTATCTATTGACATTAAGACCGAATTATTATATA
ATGGAAGTGATACCAAAAAAGGAGGAGTAAAAAATGGTTGAAAATAAAATTAATGAAATT
TGGAAACCTATAAAAAAAGAATACTTTAATAAATATAATTTTTATGTTTCTAATTTAGGT
AGGGTAAAATAAATGATAGGTTGAGTAAAGTACATCAAGACCGTGACGGATATTTAACCG
TAAGGGTCAATAATAAAAACACATGGTGCATAGACTTGTATATGAATATTTTGGCAATGA
TTTTATTAAATCAAACCATGTACACCATATAGACGGTAACAAACAAAATAATTGTATTGA
TAACCTAGAATGTATTAGCCCGTCAGAACACAATAAAAGGCACCATAAAGACAATACTTT
CAATAGGTATAATAGGGTTATGCATTAACAGAAGATGAAAGAAAAGCCATAGCAAGCAAA
TACAAGCCTAGAAAGTACACTCAACCTATGTTAGCAAAGGAATATAATATAAGTGAAATA
ACCGTTAGAAGAATTATAAAAAATATAAAGATTAAAGAAAAAGGGTTGACCTTTTCTTTT
TTCTATAGTATACTATAAGTATAGTAAGAAAGCGGTAAGGAGCCTACGTGTCCGCTATAT
AAGAGAAGAGGTTCCGGGAAAGAGTGGTTTTAAAACCATTTAATATAAATTTTTTATATA
ATAATTTTTTTATACTAAAAGGTCTTCACTTCATTATACCATAAAAAATACAACCTTGTC
AACAGTCAATTTTACACTAATTTATTTATTAAATAATTTTTTTATATAAATTAAAATAAA
TAAAATCCCTAGAAATCTAATCCTAGGGAATTGTATAATTTTTTTTATATTATTGTATTG
CACTTCTATTATATCTTAATTTCATAAATTTATTATAAGTTTGTTTACTGTTAAATATAA
TAAATTCATCTGATTCATTTTAAATATATTGTGTACAGTTTGATAGCTCATATATCCATT
TTTGCTAAATTCCATAAAGTTTTATATCACTATTATTTTTTTCTATTTTAAATTTCATAT
TATCATATCCTTTTATATAATTTTTTATTTAATTCAGAAGCGGTTATATTCAACCGCTTC
ATATTTAATTTTATTGACTAATTCTATTTGTTTTAACTTGTGTGTAAGCATATTCTAGAG
TATCCGCTATATTATAACTTTGAATACTACACCACCGCCAAACCATTTAGCAGTATATTT
TTTAAAGCCGTATAGTTTATTGATGTTGTCATAGTCTGCTAGTTTAATATCTAAATCCAT
GAAATGCACTACATAACGTGGGTTGCCGTTTGCGTCATTTGGAATTCTGAAAGCCGTAAT
GTCTTGCCCTTGTACTGTGTGAGTTTCTTCGTATAAGTAATTTTTTGTTTGTCATAATAA
TCAAATCCT

In another aspect, the invention provides a U136B bacteriophage targeting an E. coli bacteria. In certain embodiments, U136B binds a molecule of TolC. In certain embodiments, U136B binds a molecule of LPS. In certain embodiments, the U136B bacteriophage has a genome comprising a nucleic acid sequence comprising SEQ ID NO: 13.

U136B
(SEQ ID NO: 13)
CTGCTCTTTTGGTTAACGCAATTAATGAAATGATTGATGATTCAGATAAGCAGAATGAAA
CCATTCAGAAGCAACAAGATGAAATCAATGAGCTAAAAAATGAAGTAGCAGAAATGAAAA
AGATGATCGAGGAAATGCAATCAATGTTTATTCAGATTGCTAAGTAATAAAAAAGGAGCC
TTTCGGCTCCTTTTCTTTTATCAGAATGGGATATCATCATCAAAATCCATAGGCGGCTCG
TTGTTTTGAGGCGCTTGCTGGCGTGGCGCTTGTTGTTGCTGTCGAGGTTGCTGCTGTTGT
GGCGCTTGCCCTTCTCCGCGTTGACTGAATAAAAGATTAGGAAAACCCGCCGCCGTCAAT
GTGTTATAAATCTGCCCGTTGTATTCCTGGCTATCAATGCGTAACTGATCGCATGATACA
GAAATAACTTTACCCTGCTGAAATGCTTCACGATACCAATTATTCATGCCGTCGCTTTTA
GCATTAAAGAAGAATTTGTAATTGGTGTATTGTCGATTACCTTCACGATCTTTAAATGAC
TCCGAAAGCTCAACGATGTATAATGTGCCGTTTGCGCCTTGCTTTACAAATGGCTCTTTG
CGAATTTCTCCGGTGATTACGTGCATTCTTAATCCTTATTGGGGCGTTACCGCCCCACGT
TAATTTATTCAAAGTCTGTGATAGATTGAGATTTTACTTCCTGTTGCGGCTCGTTTTCAA
CATTTTCCTTCTTCGCAACATTAGCCGCTGGCGCTGCCGGATTAAAACCGCGAGCCTTTC
CGATTTCCATTTTTGCTTTTAGCTTGTCGTAATGCTCTTTAATGATTCTCCTGTTAGCCG
CGTCAGATTGCTTGTAAGCCGTTTTAAACGTTTCTTGCAAGTCTGTAAGGTTATCGCAAA
CATCAAGATCTTTTTTCCAATCCTGCGCGTTTTTGGTGGCAAGATTTGCGTCATCGTCAG
CCGTTGCCAGGCCAAGAGCCGTGCAAAGAGCATAACGTCTCGCATAGCTGTTTGTTGAGC
CGCCGCCTTGTGGATCTAATTTCTGCATTGGCAACGTGAAACTATATTCAACCCATTCAT
TTGATTCAACATGAATGAATCGCGTGAATACAGTCATTGATTTAAGATCTTCACTGACTT
TCTGATCTTGCGTCAGGAATAAACCCTTATCAGTAAGCCCAGGCATAATTGCATCAAGAA
CACTGTCAAGCGTTGCATATTTATTTTTAAGGTGCGTATTTTGCTTATCCTTTTTTACTT
TTGTGAATAACTGACGCGCCTCAAACAATGCCTTAATTACGTTTGCGTTTTGATCTGATA
ATTTCATTTCATTGACTCCGGTTTAATAATGGCGGCTTGCGCCGCCGTGTTAATTAATGC
TTTTCTTCGTTATCGTTGTGATAATCAATCAAATTCTGGATCATGATTTCACTTGCCTGA
TTAATCATCTTGCGGTGTTCTTCGTTATTTTCCATTTGAAGAACCGTTAACACAACGTTA
TCAATCGAATATGCAACCTTGCGAACATATTCAAGAGTTTCCGGTGATTCGCTTGGATCA
ATTACGCGCAAGAACTGGCGTTGCATAATTCCTAATGCGTTTACAAATCCGCCTACGTGA
TCGCGCACATCTTGAATGTCTTGCTCATTGAATTGCTGTTCCATTTTTGTTTCCTCTCTC
TCGTTGGTTGATGCGTGTAGTATACCACGCATCGAATGGCAGTTTTAGCAAAAAGTGCTA
TTTATAAAAAACCTTTGTATTGACGACGAACCCAATCAGGCGTTTGAAGGTCAATTTCCG
GCTCTCCGTTTGCGTATGAAGGCCAAACGTCATTTACCTGGCATTCTGCAAACTGGTTTA
TCACGCTCATGTATTGAAGTCGTCCGATCTTCAATTGTTCATCCGTCATGCGGTATGCAA
TCGGTAAATAAGGCTCTTTCTTTTCCTGCGCCAGCAATCTGACTACTACGGGGCGCGTTT
CTTCTGGATACGCTTTCTTGAATAAGTCATGTTGCAATGCCATTTTCAAGTAGTAGCCGT
GATTGAATGCCAGCCTTGCAAACTCTGACGGGTTAGCGCTTGACGTTGTTTTATAATCCG
TGATCACAATCGCTTCATCAAATCGCGTTGTTTCATATACCGGATTACCTTCACCATCAT
AACCGGAAATCACCGTTGCAAGCACATCTTTGCAAATATCAACATGGTCAAGCCTTACTT
TGACTTTCACGCCTTTGATAGTTCCGAAAATTGATAGCTCGCGTTGTGCTGTTGGGCTAT
TCATGCAAGCGTTGTGTTCAGGTATGCTTTCCAGTACGCGGCGCATGGTAACGCAAGCGT
CGTAATCTTTAGCCGGAACCAATTCAACGTTATCCGCTCGCGCCTGGCTTTCTGCGATCA
TTTCAATGAGCCATTGCACATTTAGATCCTCACCACAATCAACCATCATCTTAATCAGAT
CTGGATACGTTTTCCCGCTTGTACCAGTCAGGCCAAAAGATTTTAATTTCGCTGCCAATG
CTGCCTGGCTCGTAATCAGGTTTTCATAATCTTCCGGCGCTGGCGCTCTGCGGTACTCTT
TTGCGAATAGCTCACTACTCTGGAAGTTTGTGTGTGACTGCGTACCGAAAACAAGCGCTT
TGCTTTCCTCGCGTGGTCTGTATTTCCACGCTGCCGGACAACTTGCAAAGATTTCTGCCA
GGCTTGAACCGCTAACGTATTCAGCCGCCCAGCCTTTCGGATCGTGATATTCACCGTTGG
TTAACTGGTCAAAGGTGTAAACTTTAAAATCGCTCATTTATTCATCCTCTCGTTGGTATG
TCTGCATTATATGTGAATGTTTCATGTAGTCAATACCATTTAAGTGTTCTTTTTGTTTTG
AATACGTTTACAACTAGCATTGACAACAAAATCACATCGTAAGTTTATGATTTTACTAAT
TGTTGTTTTTGTGTTCCATTGTTCACGCATTTACGCCGCATACCATATATAAATACATAC
CATGTGATATATAACCATCAACCAACACAAAACACACAACCACTACAAACCATAGAGAAC
AATGGAAACAAGAAGGATACAAATAGATATATATAATAATAATTAAACTCTAAGTGTATG
TATTATATAGATATTTAATTTGTTCTTTTTGTTGTCATAACGTTCTCAATTCTACGCCGG
ATTAAGTTGTGAACACCGTAGAACAAAAGAAAACAAAATGCCCCGACCGTTAAGCCAGGG
CATTGATTTACCTCCTTTTATGCTTTGAATAGCAGTATTGTTAATACTGGAACCGCGTTA
GCGTGGTTCAGGTTGCCACCATATGAAAGCGTTGTTGCGTTACCCGCTGGTAACAGAAGG
TATCCACTTGAACCGCCATGCATAGCCGTAGCTTGAACGTTAGCCAAGCGACCGCCATAT
GATGAATCAACCCAAACAGTACCACCGCCCCACGTACCGCCTGAATAGGTATAGCCATGC
AAACCAACATAAGGAATCACAAGGTGTCTGTCGTAGTTTACTGCCGGAATGTGAACCGAT
CCGTTAAACCCAAGCACAACAGCCTTTGCAACGTCGCCCTCGATTTTTTCAGCGTAAACA
GTACCCCTAAACCAACCATCAGTAGCGTAAACGTTACCAGTGAATGATCCGGCGTTTGCG
TACACTGTACCCCTTACCGTTATCTGGTTAAATTCAGCATAACCATTCTTATTAATCATC
CATCCAGCCGCGCCGCTGCTCCAGTTGTTAGAGTTAATCTGATCCGCAATCTGAGCGTTA
CCAATAGAGCCATTTTTCACAAGCAAGCTGTTGATAAACACCTGATTATTCTCAACAACA
AACGGTAGTGTAAAACCTCCGCTCTGTTGGTTGTTTATGATCGCAAATCGTCCAGCATCA
AACAGGAATTGTGATTTAACTCCTGATCCGTCACCAACAAGGGAAAGAGCCATACCAGCG
CTATATTCCTGCCCGTTGTAACGTAACCCCAACTTAACGCCGTACATCGCGCCTACGCTA
TCGGCATTAACCCAAGAATCAAGTTTCTGGTTAACCGCTGATTGGGTATCACCAATTTGA
GTTGATAACGCCCTATCCGCTGACGCTCGCGCTTCCGCTTCATTGGTGATTGCCTTGTTA
ACATCAGTTATCTGTGCTTTGATATCCTGCTTAACACCATCAATTTCGCTCTCAAACGTA
GCTGTAAGCTGATCAATCCTTTGGCTAATGGTTTCAGTGCTGTTAGCAATTACCTCTCTC
AATTCGCTATTTTGAGCCGTTAATTTCTCGTCAAATTGCGCACTCATTTGAGTGACCTGC
GTTACCCTTGCTTCTGTTTCGTCTGCAATGAGTTTCAGCGATTGCAAGAACTCCGCTTTA
CGCTTGCCGTTTTCAACTCGCATACGTCGCACATCTTTATCATTCGCTAATGCGTTTTCA
ATCACTGATTCCGCTGTGCTGTGGATCTTATCATTGGCCTTTATTGCGTTTTCCTGTAAC
CACTTATATCCATCAGAATTTTCAATATCGACCTTAATATGATCCGTGATAATGCTCGTA
TCGTCGGAAGCCATGCCGCGCACAAAGTCAGTCCAATCAGACACATTCCCGATTTTGTCA
ACTGCCCTTGCCTTGTACCAGACAACATGACCCGCTGGCAGTATTGAATGCCAATACTCA
TATTGCGGGAACGGAACAAGCGTTAATAGCGTTGCTTCGTCAACTATAGGATGACCATCA
GCACCGTTTGGCGCTTGATGCAACTCTATATAGGCCGTGTCTCCGCTTCCCTCCGGCATA
CCCCATTTTACGCGAATTCCGAAAACCTCATTATCTGACGCTGTAAGGTTAATTGGCTTT
TCCGGCTCTCCCACTTTTCCGGTTAATCCTGCGCTTACTATTGCAGACCATCCAGACGCT
GAACCATTAGCCGCAACAGATCTAACCCTTACGTGATAGTTACCCGCATAAATACCTTCA
ACTTCCGTTTCCTTGTTCGCGGTTCGCGGCGTATTGTTCCAGTTGCCATTGTCTTTTCGC
CACTGCATTTCATACAGACTTGCGTATTCAACTTTATCCCATGAAACGTGCATTGTTTCC
ACGCTCGCGCCCTGAACAACTCGCGAATAAGATTCGATCTTGACGTTTTCAGGTGCTGGC
AGAATGTCAGGCTGCACAATGGAAGTAGGGCGATCATCAATGTTCACACCATAATCAATT
TCATCGTACTTGTTAGCGTCGTATTCAACAGCCGTGATGTTGTATACAAATTCTTCATCA
CCATCACCTTTAGTTATTCCTGTTACTACATAGCGTTGCTGGGCTATATCTGTGCGCTCA
ATTGCAAAAACCGCATCCGGCTTAACGTCAAAGCCAAACGTTGTGTTTAGCGTTAGCGTT
TTGCCGTCATCGCTCACACGCGCAATGGTTCGACCAACTGGCTTACCATCCGGCTTATTT
ACCAGAATGCGATCACCCGCTCTTGCGTCAACCTTAAACGGCGTGAACACCTGCAACCCG
TTAACCTCCATCAATCGACCTGATAGATTCAGCGTCAAAGCGCTTGACCAGAAGTTATCC
GAAACAACAATTACATCGCCTACCGTTGGGATCATACCTTCCAGGCCAGTAGCAAAGTTT
ACTGTTGTGCTCTTGACGTTGGTTTTCAGAATCCAGCGCCCACGGCGATTAGCCTCACTA
CGTCGAGTGCAACCGATAGCAGTTATTGACGTGCTGTTATATCCAAAGCGTAACGCCGCC
TCCGTTTCGAAAACTCCCTCAACATCCTGTTGATACATGTTTTGTTCGTCGTCGAAAGTC
ACGTTACATTGCGTGTACATGCTTTTCTCGCTGGCAAACGTGTAAGTAAACTCACCATTA
ACCACGTTGTCATTGGTAAAGATGTATGACGCATCGCGCGGCTTATCGATCACAATTGAA
AGGCTTTCACCGTTCCAAAAACTCATTCCTCGAAAGATTGAGCAAATATCACGGACAAGC
TGATACGCTTCTACCTGGCTCTGGATCACAACGTCGCAAAGATAGCGCGGCTCTGTTCCG
CCTTTACCGTCTGGAACCATCTGATCGCAATACTGCGCACATTCGTAAATGCTCCATTTA
TCAAGCGCGATGCCTAATTCTCTTTGATCGAGTCCGTAACGCTGATTGGTAATCAAATCA
TACAGAACAAAAGCAGGGTTATTACTCCATGCCTTTTTCCAGGTTCCATCCCATGACCCT
GAGTAAGTGCGGGAAATAGGATCATAATTTGACGGAACATTAATGATTTTCCATTTTTTC
TTAATGCTGATATTCGGCAACGCATTAGGGAATAATTCGCTGTCAAACTCAACATATACT
AAACCAGTCAGTGGGTAACGAAATTTAGCGTCTACAACTTCAGCATAGCTTTGCATTTTA
ATCAGATCCGTTACCCTTGCTGACGTGCTATCTGTCAGGCGACGGACGCGCAATAAAACA
CGCTCATTGAAAGCAGGTAAATCAATTCGGCGGCTTCTGTCGTAACCGCTCATTGTTTTA
CCTTCAATTACATCATACAAAACCTCTTTATATTCAGCACCATCAACAGCCATATCAACA
GCATATTCAACGCGAACGCCTGTTAAATCTCCGTTATCCTCCTGCGTAAACCCTCGCGGC
ATTAAAATTTTAATACGGATCGCGGAAAGGTTTTTATTAGTCACGGAAATAATATAAGGA
GTTGCAGCCGTCAGATCGCGAGCAACAGTAATCTCACTCGCCGTGTCAGTGAATCCCTGA
ATATAATCTTGCGTTTGTGATCCTGGTCTGAATTCGGCTTTAACATTCTGAAAGTTTAGT
GACCCGTCCTGATTCATAACCGGAACGTCATCAAAATATAAATCTTTTAATGAAAAGTCT
GGATCAACTTCACCATCAGAAACAGCTAAAAGAATTCTGATCCTGTTAATTGAAATCAAG
TTATCTTCCATTTCAACAGGTGTGTGCGGTTTAGATGAACCGCCCTTACTGCCAGTTATC
ATATTTTTAACCATAGCGTTTAACCTTTCTTGCTATTTAACGTGCTGTCATTGTACATGT
AACAAAAAACCCGCGCAACGGCGGGTTAATATAATTAGAGTTTATCTTCTGCGTATGATC
CAGCACTAAATATAGCGCCGCCAACCGTTCTATAACCATAAGGCAACGGAACCGGATAAC
CTGCCGCCGTGGTATTAACAGCACCGCCAAACGCATAAGACGGCTTGTTAGCTGCGCTCT
GGCTTTCCATTCTCATTCCTCCTTGTTGTGGGCTAATCATCTGCATTACTCCGCCAAGAG
CCATTGCACCACCCATCATAAATGCAGCCGATGAAAAAGCACCCATAGCCGCCAGCGATG
CACCACCAGTGAAAAACGCAGTGACCATAATAACAGCACCCAATACAACCTGAAATAAAC
CGCCGCTTTTTGAACCTGTTGGAACCGGAACAATTCGAACCTCGCGAGCGCAACGCCATG
CGCTATCATTATTCAAGCCAACATTTTTACCATCAACGAAAATTGCAAATTTCATCTTTG
ATCCGATTTCACTTTGCATAAATTCTTTGAATCCTTCAACCTGGCTTGATAATGCCCTTA
CGCATTCAGGCCAACTCTCAACGGCGTATTTATGGAAAACACCAAAGCGGCGACCTAATG
AGCCTGATAATTTTACGTTGATAACCTTATTCATTTTTCAGATCCTTATGTCTGCAAATC
ATTGTTGTGTGTTCCTGATACCATCCAGAATATAAATCAACTCGCGACAACTTGCCGAAT
GCATGATGCAATAGTTGATTGTTGCCTAAATAAATTCCTGCGTGGTTCCATACTGGCGCT
TGCAACTGCATTATGATCATATCACCTGGCTTTGGTGGCTCTCCTGTTTCTATAAAACCT
TCTTTCAAGTAATTATCTTGATAAAGGTTTTCACCATGTTCCGGCTTCCACCATTCATAC
GGCTTACGGAAATCATTCAGGATCACGCCTTGCTCTTTATGCCATGCCATAATAAGACCC
CAACAATCATAAGCACCAAGCGACCAGGGGCGACCAATCAAAGGCCGTGATTGTGGCTCT
ATGATTCTCATGTCTCCTTCTGGTATCGAAACGATAACCCAAGAAACACCAGTTTCATCA
CACATGCACAAATCGTGTGCGCTAGGGATTGTGGTTGCCCCTTCCCCTGTGTGGCTGTGA
ACTATGGCGATAATATCCGCGCCGCTATCCTCAACCTCTGCGTATTGTGTCGCGTCCATT
TCAAAATGCTTTTCCTGCTCTTTGCTTACGTTATCGATCCGGTGATATTTCTGGACGCGT
GATTTTTGAGTTACCAGGCCGCAACATTCATTAGGATAAACATCATTAGCGTGTCGCATA
ATTTCAAGTTTAATTTTTGCATTAATCATTGGTTTTTCCTCAACAGTGAAGCAACAGCCG
CTCCGCCGAAATCTAATTCATTTTCAGGACCAAAACGCAATTTGCAAGCTGTTACCGTTC
CGGCGCAATAATCCTTACTCGGATCTGTTACAGGATTATTATCTTTATCGAACATTGCAG
ATCCGTTATATCCGCAACCTTTACCGCTTCTATACCATCCACGTTGCGCCCAATAACAAA
CGCTTTGCGTAAGTCGTGGCGGGATCATAATTCCATCCATATCATAAGGTGATGTTAATT
CAAAACGTGCAACCGTCTGATTAACAAAATTTGGACGCTCAATATAATAAACCAGTTTTC
TATATGCGCCGTCCTCCACATTACCTTCATCATCAATTAAATCAGCCGATGTAACCCAAA
TTGTTACTTTGGCTTGCATCATGCCATTATATGATCTGATTAATGCAGATACGCGGCTAT
CTAAATTTGAAAGCGCTAATTGTGGTTTCTCTGCCTTTCCGTTACTTGTAAATCCTATGC
CAGTAATCCCAAAAGGCCGCGCCCCGTAAACCTCACCACGGAATTTAATGTCTTTAGGCG
GCAATATTCCAGTTTCACGCGCTTGCATTAATTCCTCTGGCGTGTACGCGATATTTTCAG
CATGAAAGCGGTAAACCTGAGCACCAAATTTTGTGCCGTCAACCTCGACAAGAGTAATAA
TCTCACCAGGAAAAAGCGATTGCAGACAATTTTCAAAAGTCTTTTTGATCTGTTTTTCAT
CCATATTTTATGCCCTCCAATAAAGCACCATTATAGACACAATAAAAAAGCCGCACAAGG
CGGCTTTGATTTATCGCATTGATGTAAATTCTTCCGTAAAAGTTGCTTTGACTTCCTGAG
CGGTTCGGCTAATTGGCGTTAGTCCAACCGTTCCGGCTTTTACGGTAAACAACCCAAAAC
TACCGTCTGGAGTTTTCCACAAGAAAGGCTTTAACCTGTGATCCGTCATAAATGCTTTAA
CAGCCTTATAATCATTGCCAACATATACAATAGAAAACTCCCTCCTTTCCGTGTTAAATC
CAGAAGATGCCTTTTGGCGATAGCCGTTACCAAAAACAACTTCCCTGTCGTTATTGGTAG
TTGTCATTACACCGCCGCCGCTCTGAATCTGCGTACACCAGGTAAATTCATCTAACATTA
AGCCCTCCCATGAACAAAATTATAAACCTCGCCACCTTGCGAGCATGACCGTTGAATCAT
TTCAGTGAAAATCATCTTGATACCAGTTTCCATCCCTTTTGGATCTTGACCGTTGTCAAT
GTTAACGTTGATATCACCAAATGCAAATTGCGATGCACCGCGATTGATGCCAGCGCCGCC
AGTTGTCACGCTTCCTACTTGACCGCCGTTTGCATAACCTCGCATCATGCGATACAGGTT
TTTAGCTCCGATTCTTTGCGTTGCCTCTTTGGTGAAAACAAACTCACCCTTATGAACAAC
GCCAGCCGGATCATATTTGCCACCGTCGCCAGTATAACCGCCGCCAGCAAAACCAGCACC
GCTTAAAAGGCTTCCAAGTGTCCAGGTTGAGCCGCCCATCATGCCGGAAATGCTATTGAA
GATAACCATCTTTGTGATCATCTGAACAATAAGGCTAATGATCGATTTTGCAAAGTCTTT
GAAGTTTGCTTGTCCAGTAGTCAAGAAATCAGCCATTTGTTGAGATAGGCCGTTTAACGC
CTGGCTCGCAATGTTGCCGATATTATCATACATGTTCATTGCTTCTTCGCCGTACTCCGC
AAAGGCATGTTTTGCACCAGATAACCAATCAGCACGTTTAGCATCTTCCTCTTTATAGCG
GTTGTCTAATTCATCCATCATGGCGGTGAGTTGCGGATCTCCTTCCTTACCTCCTTTGGC
AAGATAATCAGCCTCAACCTTTGCACGTTCAGCCGCTCTTGCTGCTTCACGCTCGCTTAA
CCCTCTGGTTTTGGCTAGTGCTTCTGTTGCTGCTTTCTGCTGCTGGATAAACTTGATTGA
GCTATCTTGCAACTGATTAAGTCGCGTTTGTGCAACAATCTGATCTCCTAAAATAGCTTT
TTGTTCAGCCATCGCCAGGATCTTATCTTTGTTTGCTAAAATGCTTTTTTCTTCATCAGT
AAGTTGCCGTTTTGTTGAGGCCTCTTGCAGAATCTGAATTTTAGCTTGTTCAGCCCATAA
AGATTTACGTTGCTGGCTTATGGTATCTGTGATCGTCTTGTGCTCTTTCAAAACTCTTAA
TTGAGTTTCTAACGCCAGGATATCACGCTGATATTGTTCTTCCAGCTTATCGCCAGCCGA
TACCTTAACTTTTTCAGCCTTTTGCTTTTTATCGCGTTTGGCTAATTCCTCGGCTTCCTT
CCTGATATTCTCCTTTGTTTTAGTAGCATATTGCTGTTCAACCGCCGCACGATTTTTCAT
CATGTCAATGTAACCCTGCTCGCCTTTTTTGACTCGCTCGTTACGTTCTGCAATTGATTT
TAATAGCTCCTTGTTGTTCTCTTTGGCGTTATTAATGATCTTCTGTTGTTGCGAAATTAC
AGAATTGCCAACATTATCAAGACCTGGGATTTTTTGCATCGTCTTAGTTGCCGACACAAT
AAATTCACCAATGAGAATATCACCCTGATTAAGCAAGTATCTAACCTGCTCAACAGTTCC
GGCTACAACGTCGATAATCAGGTTAAGCGCCGCCAGCGTGTGATCTCCAACCCATCCCCA
AGCATCTGACGCCCATTTTTTAATGTTCGTCCACATCTTTTCTAATGGCGTTGCGGAGTC
TGCCAGTTTAGCTAATCTCTTATCCATAACATCAGCAAATAACTTTGTCGCCGCCGTCAC
TGCTTCCGTTTTTCCTTTCGTTTTCTCCAGGTCTGCAATATAAGTTAACTGCCCTTCGCT
TAAAAAGTTGAATTGCTCGTTTAGCTCTGCAAGACCTTTAACCGGATCGCCAGCAATCTT
ATTAAAGTAATCGGTTATCTTGTCGCTTGATTCACCCGTTACCGTTGACCACTGAGCGGT
AGTCTTTGTGATCGATTTAATCTGATCAATCGTGAATTTGCCGGATTTTGCAAGCTCTGT
TGCGATGGATCGGATGTTTCCTATCGTCGCATCAGTGCTGTTATTAATGTCCTGCGCCAG
CTTATCGAACTGTGCGCCGCTTGTGCCAGCATAGCCACCAGTCAGCACCAACGCCTCTTG
CAAGTCACGTTGCGACTTATACGCATCATAACCAGCCTTAACAAGAGCACCCATTGAAAC
GGCTAATGCGGCAAGGCCAACGTTTACCGGATTAAGAAACGACAACAGCACTTTAAACGT
ATTACCAACGCCGCCGAAAGAATCTTTAATCTGCCCACCCTGCTGGATTGCAACCAACCA
AACAGGCATGCCGGAAGCCAGCGACGTTACAACGTCAGTGATCTGAGCCGGAAGTTGTCG
CATTGCCTGGTTATATTGCCCCGCGCTAATTCCGGTTAATTTCATCTGGCGCTCTTGCGC
TTTAAGCTGTGCAATGAATGGCGCGGCCTGAGCACTTACGCCAAGTTGCGCGGCTTTCAT
TTCCAGTAATTCAGATTTGGTTTTTCCAAGAGCCGCCGCCTGTGATTCTAGATCCGCAAG
GAAGCTCTTTCCTGCTGCCGCTGCTTTCTCTTTGGCGCGTGATTCTTCCAACGCTGCGCG
GCCTTCTTCCGTAAGAGCCTTTTTGTTGCGCTCCAATTTACTGATCTGAGTTTCCAACAT
GCTCCCTAATTGGAAGAATGTTTCATCAGGTACAATCCCTTTTTGCCATAGCTTATCAAG
ATCACTTGCTGCCTGTGACAGCTTGCGCATTTTTGCCGCTGTCGGATCAACTGTATCCTG
AATTCTCTTAAATTCACGGCTCTGTTTCGCTAATTCATCGGCTAACTGTTTAGCCTTTTG
CTTTGCAACTTCTTCCTCGTTAACAAAGCTCTCAACGCCTTTCTTTGCTTCGTCGTTGGC
TTTTTTAAAGTCCTGTAACGACTTAACAGCTTTGTTTAACTGCTCGACGTTCACGCCAAG
TGTTAAACCTGCAAATTGTTCAGCCATATATTTTACCCTCATAAAAAAAAGCGCCCGTAG
GCGCTTTATTTGTTATTTGCTTGCATCATTTCCAGTGCTTTTTGTTCCATGATGCGCAAG
TCATTTAAGGCCATTTCTTCATCTTCTATTTTATAGATTCTGAATAACATAGGCAAAACA
TTATAATCAAAACCGTAAGCACCAGCGCCAGCACTACGCCACTGTGTACCCATCGCGCAA
AATATATCCCATGATTGCATCATACTTTCATCAAATATGACCTCTGGCGGATCTTCTCCT
TCATAGTCTGCGCGGGTTAATCCTACGGCTTGCAATTCTGCATCTGTCGGCGGTTTCTGA
TAATACAGATAAACCGCCCGTTTTAGTTTTTTACGCGTTGCCCTGCCAGCGCACTTAAAT
AAGATCCCATAAGAGCCAGCGCCGCGCCAGGATAATAATCAACTAACAGTTGCGCGTTTT
CTTCGTTGAATTCTTCCTCCAGATCCCAACCCACGGCAACATTCATGATGAATTCAGGAT
CTTTAATACCTTCTTTCTGGTAAAGCTCTTGAATCTCGCTGGCCTTTTTATGCTTAACGG
TAAACACAATTTTTTGCTCGTTACCATCCGGCAATACAAATTTAACTGGAAGTTTGAAAT
CTGGAAGCGGGGCAAGAGTCAATTTCATTTTAGCCATTGTCATTTCTCCTGAGTGGTTAA
ATATTGAGGTGATATTACATCAAATAAAAAAGGGACGCAATGCGCCCCTTAATATTAATC
CTGGTTAGCAGGGAGGAAAGTAAAGCGACCTTTCAGGGATACCGACAAAGAAACGGTTTC
CATTTCGTTTACCGCCGTTTGTGGGATCTCGTTGAAAGAAAGCACACCAGCCCAAAGGCG
CATTTCTTTCGCTTTTGGTACATACATGCGCAATGCTTTAACGTCGCCGGATTCGTCAGC
CTTACGCAATAGCGGATAAATCGGGTTATCGTATTCATGCGCAAATGTGTAAGTCAGTGA
TACCGCGCTTTTATAGGTTGGTAACTGCTGCTCCTGGTCATCGCTCAAGCACTGGTAATT
GTAATACTGTTGCTCGCCTCCGTCCTGCCCCAAGTCCTGTACGCATGGGATCTCTGTCCA
ACCGTTGATTTTCGCAAAGCTCGCAGTTACAGCGCCAGCCGGGAAAACGTTAGTATCGGT
TGTGTCGATACCTTCAACAGTAACCTTAGTTTCTTCAGCCGCCGTCACACGCAGAACGCG
ATCAAGCAATTTACCCCAACTTGAGGCGGTAACGATAATGTAATCACCCTTTTTAAAGTC
AGTTCCGCCACTTGCAACAGTGAACTCCGGTTTTTCAGCGTTGCTAACGTTGGTTGCGCT
TACTGCCTGTGCGCGAGTATTCTCAATAAAAATCTGTGCTCCGTTAGGTAAGTGCATAAT
AGCCCCCTTTATTAACCATCATAACGAACATAAAAACGTACTGGATAAAACCAGCCTGAT
TCTGATTTCTGAACTTGATGCACTTTTGCACCTTCTGAAATATAGCCATTCGAAAGCATT
TTACCATCTTCAAAGAAATCAGCAATATTTTTTGCCAGCTTGCGAGCCTTATCAATTCCT
GAACCTGGCGGGAATTCAATTCCGATCTGAACCATTCCAATATAGCTAATGCATTTCCTT
TTCAGATCATGAATTATTGTATCTGCTTCTTTGTAGTCATATTTAAGCCAAATGCCTCCG
CCTTTTGGTGGCGTAAATTCAACGTTTTCATAAGCTATCATATATTCGCTTTCGTATTCT
TGAGCCAGCGCCATTCTTGCCGCAACAGATAATTCATAGTGCATTTTTCTTTCTCGCCTC
CCTTATCGCTTCCGCCATGTAAGAGCGCAACCGGATCGCAACGATACCAAATACCCCAGC
TGGCGCTTGCTTTGAATGACCATATTCAAGAGCATTAGCGTAAATCAGCATATTGGAAAA
GTATATTGATTTAATAGCGCCGCCGCCATGCAATAAAGCATAAAGCGCCCTGCGACCTTC
AGCTTTTGTTTTCTCACCATCAGGATCATATTGATTGAGCGCGTAAAGAGGCGGCTTGTT
TGCTGTGATCTGCATATTGGCTTTGAATCGTCCTGTATCCACTGGCGCAATATCAACTAA
TGCGCCGTGAACCTTTTCACCAAAAATCTGAATCACGTCATTCAATCCGCTTTCTACTTG
CTCGATCCATTTATCAACATTGCCATGAAATTCTCGGATCGAATAATCAGCCATGAACCG
CAACCCTCCGCATTATTGGACGGTAAGCAACGGTAATGCTTGTTGGTCTGATTGGTCTTG
TTTCAACCATAACATAACGCTCGCCGTCGATATCAATCTGATATCCGTTTTTAAGCTCTA
CATCAGCATTAAAAACGCCTAACTTGTCGGTAACTCTAATTGTCTCTCCGTCAACTTCTC
GCGTCCGTGGTGCTCTCACAAGACCTTTAATCTTCACCTTTATTTCAGGTTTTTCAACCT
CAATACCGCCAACAATTTCAACGCTTCCAGGCTGAGTAATGCAATCAAATTCACCAGTTC
CATCGCTAAAAAAATTAATTCCAGCCGATGCCATAGATCTGATTTGTTCATAATTCATCG
TGAGCACCTCCGACGAAAGGCAGTTGTAAGGCCAAATCCGCCGCCTTTTTTCTTGTTAAG
AACTTCATACATTTTACCCCACGGCGTTTGGCGCAACGTGTTACCGCTTGTGTCGTCACT
AACTTTGCTGAATGTCTGAGAAAACTCACCAGTCAGGGAAAATGACGCTATACGATGCGA
GTAGCTTTCTACGCTTTCACCTTCCTGTTTCATTGCGCCGTCAAGAGTCATCAGGTGCAA
CGTATAAAGAGCAACAGCGCGATTGTACTTGTCTTTGAAGTGGTTAACGCAAACGAAATC
TTTTGCTAATTCGATCCATGCCTGAATCAATTCAGGATCTACTTTTTTAAATGCTGGAGC
AAGTTTATAAATCTGCTCTAAGATCTCAATATCATTCATGATTTACCCCCAATAAAAAAG
GCGCTAAATAAGCGCCTTTATTAATTAATACTCGCCGCCGTCCTCAAGCTCTTTTTTTGT
CTTGCCTTCGTTCGGATCTTTTTTGCGCTTGCTCTTAAACGCTTCAACGATTTCAGCATT
GAGATCGCTGTTATCTTTGATTGTTAGCTTACCTTCTGCCATAAGTGCTTTAGTGCCTTG
CCGCTCTAACACGCTTTCCGCAACGGTAATTTCTTTACCTGGCATAACGTCAACGCGATC
AATAATAATCAGTGCAACGCCAGTGTTAACCAGTGTAATTTCTTTTTCTTTAGCCATTTG
TTTTTCTCCATATATAAAAAAGGGAACCTTTTAACGGTTCCCTATATTAATTGAATTATC
AGCCTACTACAAGACCTTTAATCAGAACCAGCGTCAACGGACGGTAAATCGTCAGGCCAG
TACATTTAGAGGTGCAAGGCACTTTGAAATGCAGGTCTTTAGGTTGCGCGGTCAGCATGT
TAAACGCTTCTGGAATCTCAATGCTCATGTTCATCGGATCTTTTTCGTAAACCAGCGCCG
CTTTAGTCCCTGCGCCGTCAATATCTTCAAGCTCGGAAATGGATTCGATAGTGATACCTC
CATTCTGCTGCTTGAAGTAATCAAGATAGCTCATGGTGGTTTCCGGCATACGAACGCTCA
AAACCTTACGCATTGACGGCGGGATCAGAATGTGAGTAGCGCGGTGCTGCCCGTTGGTCA
GCGTTTCGATTTTTTCAATCGCTTGCTCTAATTCATCCTGTGCTGTTTCCGGCTTTTTAC
CAGTACCTGCGGCGTTGTTCCAACCTGCGGAGTTGATAGTTGTGATGTTCGGATGTTCAA
ACACACTGATAATTTTGTGCGGCTTAGAGCCTTTGAAAACCAGGTGATTGACAAGCTGAT
CATGCGCATTCTGTGCGGCGTTTGCCTTGCGAGTGGAAAGGCTCTTGCCAGTGCGTTGAC
CTGCTTTGATTTCGTCAATGGAAATCAGGAACGCATTACCAAGTCGGAACACTTTACCAA
ATTCAGAAGTCATCATCGCGTCAACAGTTGGCAGATCATCAGTATAATCAGCGATGATTT
TTGCGTGACCTACTTTGTCGAAAGTCTGGTATTCAAAAGTTTTATCAGTATCGGAAAGCT
CGGACGTTACAGGGAATACACGCAGTGCGGAACCTGCCGGATATTCTTTCTCATAAGCCT
GAGACTTAATTTTGTGTAGCTCTTGAGCCGTCCAAATCCCCATATCAGACGCATCGTTTT
TAGCGCCTTGCAGTTGCAGGTGATTAGAAATCACAGACGCTTCAAATTCATCAAATTTAA
TTTTTTCTTTAGACATAATATTTCCCCTCATAGAAAAGCCGTCACAATAACGGCTTTAAT
ATAGCATTTTTTGTTAAACGTTCAAGTGTTTTTTGTGTTAGCACGCTACAACTGAACTGA
TAGTAAAATATATCCGTGTGAATATCTTACGTATTCGCCAGTAAAAAACCAACTAGTAGG
AAATCCACTTGTTGATATAGAGCCATTGTCTTTAACAAAAACAGGATCTTTATACGATGG
AATATCATGTTCGTCCTGAGTGATTACCCAAATTCGACCATGAGTTATTACGTTAACAGG
ATCTCCGGCGTCGTAATATTCTTTGCAAAAATCCATATAATCACGCGTAACGATTCCGTA
ATGCTTTGAGTTTTCATTAAAATTGTGGCTGATTATTTTGTAACCGCAAGATATGCGATC
AACACATACGGCCTTACCTATCACGGCTGAAACATCCATAACTGGCGAGCCATCTATATT
ATAAAGGCTTGAATCTACCAAGCACCCATTTTCAACTATACCTATTATAGAACCGTAATC
AGACATAAAACCACCATATATAAAAGGCCGGAATAATCCGGCCTTATTATTATACCAGTT
GAACCTCAACGAGTTTTAGACCTTGGAATTCCGTAAAGTCTCCAGTTGCGATCCAAGTGG
TTTCAATTAATCCAGACGTTAAATCAACCTGTCCTGAATCATCAAGTTTGATAGCTGTTT
TGAATGCTGGCGCTTCCGACTCTACAGATAACATCCAAACGCGCCCTTTAGTCATTACGT
TAATTCCGCTACCAGCTTCATAAACCATGCGACCATCTTTAGATGTGGTCTGGAAGTTGG
ATCGAATAGCAACGCCGTAAGCCTTGCCGCTTGCCGCCATCGGCTTAATCAGCTTATTGC
CCATGGCATCAACGCCGCCATGCTGGACCGCCACACCAACAAAAACAGGCTTATTCTGAT
TGCCTGCTGCATTCTCAAGAACACAAGCTCCGTCGATGTTATACGCGGAAGTGTCGGAAA
CTTGACCAGGCAAGGCGCGAGCCATTGCAACATGATAACTTGCTGAAATCTGAGCCATAA
TATTTTCTCCTTATTTAGCTTTGTTCAGACGCGCGGAAGGATTTAAAACAGAATCCGCGC
CATCTTCTTTTTCTTTTGCTGAATCGCCTTTCAGTGCAATACGTTGAGCCGCCATTTTAT
CAGATTCTTTCGCGATATCAAAAGCGGTATCAATATATGCATCAGCCTTTGCGCTAATGT
CATTACCCATGACCTCTTTCACATATGCGATCTTAATTTCTTTGGCTGTTAGTCCGTCGC
ATTTTACGCCAACTTCGGAAGCGGTAGCGACCAGTGCGGCATGTTCATCAGCGTCAGCTT
TTGCTTTTTTCACTGCTTCTTCAATCTGCGCCGGAATGCCGTCAACTTTCGACTGTAGCG
CGTCACGCTCTGCCTCCAGGCTGTCGGCTTTGGTTTTGGCTTTTTCAGCATCTGCCTTTA
CGGTTTCGATAAAGGTAGCAACCTCTTTTGGTACGTCAAATTCAACAGCACCATCCAGTT
TAATTTTAACGGTCATAACTTCGTCCTCTTTATTTAATTTAACGTCAGTATCATACGGGA
ATTCCTGCTCGCTATCAAGATTCAATTTTGCAATTCCGGCACGACCTTTAAAAACAAGCG
CGATATGGTTTACGCTAATGTTTGTTTGTAGCGCATCAAACTTAACCCAATCTTTCGGCG
GCTGCTCACCATCTTTTAGATCTTCTTCAAAGATGTATTCGCCCGTTTCATTAGACCCCC
AACCTGGCTTATCAATATCAATTGAAGTATAGCCAACGCTGATTTCAGCCGTTTGTTTTT
TCTTCGCGCTTTCAATTGCATCCTTTGCGTAAATATTCAAAGGCGCTTCAACTCCGATCC
CATTTGGAATGCCAGCGCCTGAACAAGTACCAACTACAACATCACGCGCATTTTCCGGCG
TTACGGTGACGTGACCTACTGTGATTGGTTTACCTGCGTAAGTCGCCAGGCTATCAGCTT
TAAACACTTCACTTGCTGGCCTGAATTCTCTGCGCTCGCCGTATGGCGTTTGATAAATCT
GCAAACCAATTCGCGCCACAATAGGACGATCCACTAAAAAGCCGTGTTCATCAAAATGCG
CCTTTACTCTGACACTATCAAAACGTTGCACCTTCTTCATAAATATACCTCTTGCTCTGT
TTCCCAATCCGGTTGCGCCCAACAGCGACAATTATATTCCTGCCCTGGGAATATGTGCTT
TATTGCCAATGAGATCCGTTTCATATCCAATTTAGCGTGTTCAGGTCTAACCCTGTTATC
TTCCTGAGTGCGCCAAATATAATGGGTAACTCCGGTATCAACCACGCGTTGATACATTAA
CACGGAATACCACGAACCAACAAAACCAGACGCCCTATTTTTAGCCCACGCCTTATATTT
TCTTAGGCGCTTTCGGATCTGCGCTCTTGTTTCTGCTTGCTCGCGTAACTCATTTAAACG
CTGTTGTACTTCCCAATCATCAAGCACATTATACGCAAATTTACTTACTGATGCCTCAAT
AAGTGATCGCCAGGTTATGTATTTTGAATTGAACCAACGTTCATTCATATTTCCGTAAAC
GCCACCAATCAGAAGGATAACGGATCGATTATTTGCACCTCCTGTTTGCTTTGCCATTCT
GATAAATTGGCGAGTGTGGTATTTGTATGCACTAACGGCGAACCCGTAAACCAGCAACAT
AAAGGCGGCAATTGCCGCCTCTACATCAGCATTGTTTTCAGTGATATCAACCGCCAGATC
ATTAAGCGCCTTTTCAACGTCGCTCTCTGTCATATCGGGAAACATCATTTATTCAATCCC
CCCTCTTGCCCTGGCTCTGGATCTAAATCTTCCGGCTCTGGCAATTCAATGTTGTCATTG
TCCATGATTTTAAGATCTGGACAAATGGATCGCAAAGTGTCTCTTGTTTCTTTGAGATTA
ATTGCCTGTTCAGCTTTCAATTTTACAACAGATTCAACGTTCTTAGCCATAATTTCAGCT
TTGTCCTTATCGCTCGGAACGCTTAACGGTTCGAATTCAATAGACCATTCGGATTCTGAG
ATCATGAACGGCAAAAGGAATTCAAGAATCGGCTTGTAATCCTCTACGCGCTTGCGATCA
ATCAACTTATAGAAAGTCTCTAGCGCCGTGTTTTGACTCGCTGAAACTCCGCCAGTGTTC
TTATTTTTAAGGATGATTTCATGAATGCCAGTTAGGGCAACAATTCGATCAATCTTTTCC
TGTAAGAACTCTGGAACGCCGGAAACGTCAGAATTGAGAACCTCATATTCTTCATCTGTG
GCATCAATACCAATTGCCTTACCAACTCCGCTTTCGTCATCAACCTGAGCCAAACGCAAA
CGCGCCGCGTAGCGCCCTTCTTCATCGTCGCACATCAAAGCCAGATCACGCGCTTTCCAT
ACTGCTTGTTGTTTTCGGCGTAGCAATTGCGTTGCTAATTCCTGGCAATAGTTATAATCA
ACAATCGCCTCAATCAAACGCTTGTTTAAAATGCTTGCGCCCCAACCATCATTTTGACGG
CGTTTTTCATTCGATACTCGCTCACCATCAATGATGCAAATACGCGAGTAATGAACAAAG
AATTCCGGTATATCTCCGCCAGGTGAGATCTTATAAAGTTTCGGCTCACCGTAACGAACA
CTCCGCGCATTAGTTTCACGTTCCTGAATGGTGATCTGATAGCGATCATATACGCGAATA
TCTTCAAGTTGTGCACCAGGCTTTACAGGTGATTTCAGCATTCTGTTATCAGCAACAACA
GCAAGAATTGCAGAACCGCCAAATAAACGCGACCATGAAAGCGCATCAATAATTTTAGCG
TTTAATCGCAATTCATCCCAGCGCGATTTAAAAGACTTTTCATTTTTCACGCCGTCAACT
TTAAAACCAGGCGTCACCATTTCTTCTGGAATAACATCGACAATGCGCCGCGCCATTCCA
TCTTCTTCATAAAATTGCGATAACGTTTTAAATGCCGCACTTTGCATGTAAAAAGCATTT
GGCCTAAACGTTCCATCTTTAGATCCGAAAATTTCGTTGTAACCATCTTCTTTAGTAATG
GCCTTTACCTTGTCGCTCATAAATACCCCCATAATAAAAAAGGCCGGAATATTCCAGCCT
TTGATTTTACACATTAACGCTTCGCTAATCCAGCTAAACGCTTCATTCTATCAACCGCAT
TATCAGCAAGGTTAAGCTCAATATTAACCGCATCAAAAAGGTTATCCATAATATCATCAT
GAGGATGGCTATCATCATAAGTGAATGCCGCCGCCTCTGCCAGAAATTCAGCAAGCATGT
GATGCGATTCAGGCAATACAACATAACCATTCTTGATCACTGGCTGAGCGTCCATGCAGC
GCGTTACTTTGTCTTTATCGCGTTGCACTGGCGTAATTTCTATCGGAAATGCTTTGCGGC
AATTCTGGATCAAACCTGTACCGCTCGCTTTATCTTCCACGTAAATTTTACGCAAGTTTC
CGCATTCTTTGTTACGGTTCCAGCACTGTTTAACAAACGCTTTAAACTGCGTTTCTAACA
TTGGCGCTTCCCATTTTCCGCGCACACCGTCAATAAAATATATACGGCCTTTGAACATGC
CCCAATAACATAAAACGCTGTAGTCGTTTAGCTCACCTTCTTTCTGAGCGGTATCTGCTG
TAATAAACGTATAATCAAAGCGATCCGGTTTTGGCATAGTTGATTTTTCTCCGGTTCCGT
AATACTGGAACCATCCAACGTTAATTGCGTTACCTCCAAGAGCAATTGGATCTTGCTGAT
ACTGAGAAAGGAACGTGTACAAATCCGCATCGCGTAGCGCTTTCAAGTCATGAATGCTTT
CCTTGCTAGGCCAAAAAGAATAATACTTCACGCCGTCAATAACGATGTATTCAGACGAAA
GCACATCCTTTTCAAAGTGCGGCTGCAACCAATCAGGAAGTGATTTCCCGTACTCCTCTG
TAACCATAGCCGGAATTGATATCTGATCAAATTCAATACCCATTCCGCCATTCATCAGAA
ACCATGTCATATCCTGCGCATGTAAACGCTGCTGGATAACGATAATTGGCGTTTCTGAAT
GTGCGCGACGTGATCGAATGGTGTTCTTCGCTATCATCTGACCGCGCTCACGCTTCACCT
TCGAAAACATATCATCGGGCTTTTCTGGATCGTCAAGCGTAACGCAACCGGAATAAATAC
CAGGAGTGATATAACCGCCACGGCTACCAGTAATTTGACCGCCCATTGATTTACTAATCA
TTTCAAGTCGAACTTTGCCAGCGTCATTTAAAACCTGAAATTCATCATCTTTGCTTGTAC
CAGTTTTTGCAGGGAAAAGCTCTTGCCATTCTCGCGAATTAACCAGATCACGCACACGCT
TACTGTTTCGTTTAACAAGCGTATCGGCAAATGAAATATTTAGGTTCCGCACCTTATTAA
GTTTTAAATAAGAATACGGAGGGAAATGGATAGAAAGTAATTCCGTCTTACCTGAGCCTG
GAGTTACATTAAAGATCGTATCTTTCCGCGTACCTTCAATAATTTCATCAATCGCTCTTG
CGATATAAAGGTGATGCCAGTTAGGAATCCATTTTTCCCCTTGCTGGATCGGGAACCAGA
TTTTAACGAAAGTATCAAAATCGTGTGTACTCAATTCTTTAATAGCCAGCTTTTCTAACT
TGCTTAAATCTTCCCATATCAACATAGCATTCACCCTTAAATTTTACTTAGAACGCTTGC
GACGGCGTTTTTCATTGCTTCTTCCGTGTTAACTTCCGTTTCAGCATTGGCGCTAATATT
GAACGTGTCGCCTTTATCAATGCCAAGCTCTTTTGCGATCATGGTGCTGTTAATCATTCC
GTTTGCAGCTAACTGGTATTTTTGTTCTTTGATTACCGATTCCGCAAACTCAATAACAAC
ATCATAGCCTGGTTTAAGTTTCCACTTTGCTAGTGCTGTGCCTGAAAAACCGCAAAACAG
CTTTAACCCTTCCCATGTAAAAATACGCGGCTTGTGAATCGTATCCTGATACACTCGACC
CTGAAAGGATGTAGTCTCACCTGCTTTAATGGCGTTTAGCTCCGCCCACTCAAAATACTG
AACAATCAGGTTCATGACTTGTTCCGGCGTTAGCGTTGCGCGTTTATCAATCTCAATACC
TACCAGATCGGAATATCGCTTATTCCATAACTTTTTAAAGTTTAGACTTTCCGCCGCTGG
CGCTTCTGTTTTTTTAGCCATAATAAAATCCTCCTGTTGAGGCGTGATTATAGCTAAATT
GCAGACACAAAAAAACCCGCGCAAGGCGGGTTGTCTTTTTTTCGTTGAATCAATCAATAT
ACTTGATGCTTCCAGGCTTGCCAAACTTGCGACGGAATGCAATTTCATCAGCAATCTTTT
CAAAGTTTTTAGTAATCTTCCATTTATCAGCTTTGTTTTCGTAAATGGCGATCACTAATT
CGTTGTCTTTTACGCAACGCTGCACACCCCACGAAAAACAAAGATAGAGAAAGAAACAGA
TAATCGCACAAAAACCAAAACCAGCAATGAACATCAGCATTATATAGCCCTCACATAATT
GATTGAAAATTGGTGTGTTTCAGGGTTAGCACCTGAAAGAACTTTAGCGCATTTAACCGC
ATCAACAGTGTTTAGCGCTTCAACCTCGCATTCGAAATCTTGTTTACATGATCCGCAATT
GCGACCCATGCGCCGGATAGTTAGCTTAACCTTGAATTTCATCCGGTTTATCCTCTTGCA
ATTTCACCATGAAATCACCGATCCGGCCTAACATCGCCTCACCTTGTTTAAATCGCTTCT
TGTATCGCGTTTTCTTTCCGTCACGCTCGACGATGATAGTTACCTTTGCGATTTCTTTGT
TTTGCATCGTGAATTCTCCTGAATGGTTGCGGGTTACGGTTCCCGCGATGGTCGTTTGCT
ATGCAATCCCATCCGATTAGGTTTTACGGCTAACGCGCCGCGCTTTGCTCTATTGAGCCT
CTTATGTTCACGGCGTTTAATCTACCAATCAGACAACACGCCGTCAACCTTCATTGATTC
AAGTGCTTATTATTCATACTCGCCAGGCTTACCCCATCGACCAGCTATATAACCAGCAAG
CCATACAAACTTTGTGCGAGTGATTAAGGTAGAAATTGGCGTGTGATGTTTGGTGATAAT
GCCAGCCGCAATATGATCGTATTTGTTGCCGCTCGTAACTTCAACTTTCAGATCCTTCAT
GCACTCTTTTGCAGCGCGTTTAACAATGTTGTATTCAGCCTCATTAAGTCCAAACATTTT
AAAGTCCTCTTGATTTCCAAAGTTGAAACATTTCAAAGTAGTGCAATGCTGTGTCAGTGT
CGCCGCGCTCTAATGCTTCTTCTTGCTTATGTGCGCACCACTGCGACGGTTTTATGTATT
CAGGCATAGAATCACCTTCCTAAAAAATTCGCCTCTTAGAATTCGTCTGGAGGCGATTAA
AATCGATGCAAATTCGTTAAATTTCTGCGAGTAGCTCGCGGAGGCGGTTTAGCACTCCAA
CCTTGTATAAATCCCACTCGATAGAACCTGGCTCAAGCTCACGCGCCGAACCTGGCGCTT
TCGTGCTGAAATCGTCAATGATACATGCTACTTCATCAACAACGATTTCCTTTATCTGCT
CGTTAATGCCTAAATTCTCAACCGATTCAAAAGCCTGAACAACTCCACCTAAAACGCGTT
GCTGTGCTTGCGCTTCTTCTAACGTGTCGTGAATGTTTCTGATCTGCTTGCTGCCAAACT
TGCCAACTCCGTAAGAGTGAATGTAAATCATGCTAACACCTCGTTTTTACGCTTCCACTT
TTCAACATCCATTTCGTTCAATCCGTTTTCGTGCCGCCACACTGCTAACTCTGCCGCACT
TGTTGAGCGTGGCATTAATTGCTGCTCAAGCTGGATGAAAACCTCTTTTGCAACATCGCT
TTTAACATCCAATCGCTCATATTCACCAGAGCCATAACGCCATCCCAAGCTATTTAGCAA
CGATTTCGCTTGTTCTTCTGATAGGTTGATTGCGATCATCTGTTATTCCTCCACTGCGTA
AAACTTGATGCGGTTGTTATCAACAAGAAACATAACGTCGTTATCCCATTTGTCGGCCTC
TACTTCATAATATTCCTCGCTGTCGTCATCAGCCAGGATAACGCGATCTTCATCAATGAT
TAGCTCATAAGTGCCGCCGTAAATCGGGTTTTCGCTTTCAAACATCTTGCCAGTGTATAC
AGCCATTTTCTAAATCTCCGTGTTTCGCTTCAATGAGGACACTTTAACAAATGCCCTCGC
GGAAGTTTTAACAAAAAGTGCTATTTGCACTTCATTCCTGGGTAAACGTCAAGCAAAGCA
TCAGCCAACGTCCTTACAACCTTTGCATGAGTCACTATATTCTCACCTTCCGGCACTCTC
AGCACTTCACGCAGTTGCTCTAACACTTTGACGTATTCGTTAGGCTCTGAGTTAATCAAC
GCATTGAATTTCGCATTCTCAATTACAGAATCAACTGATTTTGCAATTACGCGATCTTGA
ATTCCAACAGTCATTTCACCGCGCATTGATTTGAAGATATCGCCACCAGGCGAACATGAA
TTTCTCACCTGCTCCGCAACGATATCGCGAATTCTTTCACGCTCTGCTAATGCACTTGCT
TCATCTGCGATCCGCTTTGCATTGCTTAGGTGCTCGCTTTCGAAAAGAGTTTTATCAAGC
CAAACTCTAACGCCATTATCAGCAACAACCAGGTAAAGAGTATCTATGCTATTCAAAACG
GTATAGTCGCGGCCTTTTGTCAGGTAGTTATAAAAATCATTTTTAGCTTTCATATTTTAT
TCTCCTTACAAACAAGAAATTTCTTCTTCTGCGAAATAGTCATAGTCAAATTGACACCAA
TCCTTCAAAGGCGTATCAATCCACTTTGCGAAATCTTCCAGAATTTCGGAGTTGTAGTTA
TCCGCAATGCAGCGACCCCAGTCGCGAGCATTTTTATAACCGTAGCGGCTAAACGTGTAA
GGCGATTTGTTAGAGTCTCCCCACCTTTTAGCAATCTTAGCAAAGGCGCGTACCAGCATT
TTTCTATTCATCGTTTTGTCTCTCTGCGTAAATGATCCGAAAGTTTGTTCTTATTTGTTG
CGTCATCACTGCCAACCATGCCACCAGCAAGAAAAATAAGTAATCCTCCAACAGCACTTT
GCCAAGTGTCATTGTCAGTAAGCCATATGCCGCACACAAGCAATGCCACAAAACCAATAA
ATTTAATCATCTTTTTCGACCTTAACCGGAGTGTTTAAAACATACGTTTTAAGAGTGTGG
CCTTTTGAATCCTTAGTTATGATGTTTACCGTGTCGCCGTCGCGTCGTTCTTCAAATTCA
ACGCCAGGCACATAAAGCAAACCTTGCGGCGAATGAATCCAGAACATTTTTATTCCTCCA
CGATCTCAAATTCTGCCAGAACGAAAGAGCCAACATTTACAGATAATCCGTCTTTACCGT
TTAAGAAGAAGTGACCGCCGTACTCATGCGTAACAGTTGGGCTAAACCCGTTTACACCTT
CTTCCATATGGTAAATCGCGCCAACTGTCAGATTCATTGTGTTTGATTTGGTGCATTTGA
TTTTCATCGTTTATTCCTCTGATTGGTTGCTTCTCTTGAATAAGGCCACTATAACAAATG
GCCTTGCGCAAGTTTTAGCAATTCGTGCTATTTAAGGTGATCGCGAATGATTTCCTTTAC
CGTTACATTTGGTTGTTTATCAACCAATTTATAATCGTGCTCGCTATATCCGTTTTTCTG
GTAGTATGGATTTATTCGAATGTAGTTGCGGCTATCACCTGCAAACGTGAACCCGTCGCG
GTGCAAGCGGCATAACTTCACCTCATTACCAGCATTGATTAGCGCTATAATTTCATCTGG
AAAACCGCCATCAGTGCAAACAACAGGAGCATCACACTCTTTTACTTTCTGATTAAACCT
TACGCCGAAATAATCCTTACCGAATTTAGGCTTGATCACATCCTCGCTAATCCAGATCAT
GAACTCGCGCGGTGACATACCCATCAGGAATGTTTGCGGCCTTTCTTTCTGCTCTCTGTA
ATCGTAAGCCAGCATGAAACGCTGATAGTTCTCTTTGCCCAGGATAGCTAATGCAATATC
AAACATTGGCTCTTTAAAGCTAATGATTCGGCATTGCTTACCAGGCGAATAGACAGAAAT
CATCTTGCCAATCGTATCTTTGCCAGCACCAGGCGGCGCGTTGAGGATAATAATTTTAGC
CATCTTTGTTCACCTTAAAATATTGCACCGTTAACGTTTCTTCGATCTGACTCCACCCTT
GATTTCTAGACTCAATGCAAACATCGTTTAATGCTGCCTCGCCTTTTTCAAGTGCATCAG
CAAGAGCGAGTAAATAGTTGATCGTCTTTTGCGTTTCTAATTTATTATCAAGCGTCATGA
CCAAACCCCCATGAAAATAGAACCAACGGCAATTGCCACACAAACAATAACGGCGATAAC
GTGTTTATTCATTTCTCGCTTTCTCTTGATGCCGTTTGCCTTATTAACAAAATCCTTAGC
CTCTTTGTTGTAAGGCGCTAACGAGCCACGATTGCACATACTTACCAAAACACCAGGCGA
TTGAAATACGCTCTTGAAAAAATTATCACCATCAAAGAACGGTTTTTGTGAGGCAAAAAT
AACGCCGTCATCGTTAACAATGTAATAATGGTTAGTTCTAAATCCAACAGTGGTAAATAC
TTTCATTTGTCATTCTCCTTGCTTGTCTCGACAAGGCACACTTTATCAAATGTGCCTTAG
CAAGTTTTAGCAAAAAGTGCTATTTACCATTTAAACTCACCATCGCGGATTAGCTCGTAA
ATTGGCACGATGATATAACCAGGCCATAAAAGAATCGGCTTCCAACTATCCTTATCATCA
GCGTCAGCCTCTTTTAGTAGTGCTCGCATCAGGAAAGCGCCGCAAACATACAGAATTAAA
ACGATAGCGATCAAGATAAAAGTCAACATAATAAATCCTCTATTCCGGTAATTCTTCAGC
AAGACGCTTGATGTGTTCCGCTAACTCCAATGCGCCTTTCTTGTCAAAAATTCCGCGAAA
CTGCAAATGTGTGTCATCCTCATATTTGCAGTCAATCCACACCGATTTCCCTTCGATGTA
AATAAGCACGTTATCACCATGCCGATCTTCAATTAGCTTTGCTGTCATCGCTCAATTCCT
CCACGCCGTGAGATTTTAAGTGATCGTGTAACTGCTCGCCGTAGTCAATTACCTGGTAAA
CAGTCAAGCCCAGGCCGCGAAGGTGTGCAATAACGTTTGGCGAGTCATCCCAACATGCGA
CAATGCGATCTAGTCCGATAGTCCGTAAAACTTCCTCTTTGATTACTGTATCTTTCCGGT
TGTCCTCCGCGCGGCGCATAATTAGCCAATCGAAAGAAACGCCGTTTTCAGCTAACCAGG
CTCGCGTTTCTGCTTCAACTTCATCACTACGTCCTGTTAGGATGATAACCAGGTATCCGG
CGTCAAACATGGCGTTGCACACGGAAATGTTATTGCTGAATGGTGAATCATCCTTTGCGG
CGCGATTAAACTCACTCCAGCTTTCAGTAAGATGCAAATCAACAGTTGGCAACAGGTGCA
AGCGGTGCTCACCATTTGACAATGTGCCGTCTAGGTCGAAAATAACGAAACGCTGTTTTG
TCGCAACATATGGATTAACATAATCCCTTCCCCACAAGTTGATCGTAAACATCAATTACC
CCGCAAACTCAATAATGCTATACGCGATGCCAGCCAGCACAACGGTAGCGAGCACAATCA
TAGAACCGTATTTCAGAAACTGCTTGATAGACTCTTTAGACATTTTTACTTCTCCTTTGG
TTGGTTGATGCGGTCATTATACCCATCAGCACACATGACCGCTTAACATTTTGTGCTATT
GCGCTTCTAACTTCATAACCTTGCGAGCCATGCGCTTATACTGCGATGCAGTGATTTTGC
TTGTGTCGGTAACGTGGCTATTAACCATTACAAACTTGCGACCGTGACCAATCCAGTGAT
CATGACTGTATGTGTGATCGTATTCAACCAAATCCGGTCGGAATGTATCACAATCACCCT
CACCGCCTTTCTTAACCCTTTGGTCAACAAACATTGCAATATCAATTTGACCGCGCTCGT
TAACATGAAACAGGTGCGCATAAGTTGAGCCTTTGCCGCCTTTAAGGTGTAATCCATAAA
CCGCGTTGCGTGCTAATTCCATTTTGTGATCCTCTCGTTGTTTGGTATGGGCTTACTATA
CCGCAAGCCCAAAATTCTGTTTTAGCAATTCGTGCTATTGATCATTTTTTTTATTCTGTG
TCGGTAGAATTGCATTACGTGCTCATTGTGCCAACTGCTCACTTTATCACCTCAATCAAC
AATCCGGCATTCGCCATAGGTGAACGGGAAATGTGTGTCGCTTTCATAGCTATCCGGTAT
ATCAGCACATTGGCCTTTCTCTTTGTCATAGCCAGCCGCGATAAGTGCATCAAGAGGAAC
GTCAACCATGCCAACTTCAATACCGTAATCAATGCAATTCTCAATGCGGATCGGGAAGGT
TAAGTGTTTCATTCCATCGTAAGAGCCATCATTCAGGATCTCAACAGTTACGCGTTTCAG
TTCAAACATTTTTATTACTCCGGTATGCCAAAGATAGAAATTAATGAGTTTGTGCGGCTG
CGGCGATATTTGCGCATTACAGCCGCTACATTCTCGCCAGTTTGATGACCTAACGTGATC
GGCATCTCTGAGCCGTCGCCGTATTCATTGCCAACCTTTTTGGGCTTATCTCTCCAGGCG
TTTATAAATCCGTATTCATCACGAGTGATATAGCGGTATATGTTAGGAATTGGTAACTTG
ATCCCATCAAAATCCACTTCCTGCGTGTAGCTGCGGAAATTATGCATGGAACTCATAAAC
AATCACTCCCATTTCATCAACAACTTGCGCCTTGTAATAACCACCGTTAGCAGCAAGCAG
ATAAGCCTGGCTAAGAGTTTCAAACTCGCGGGTTACTGGCTGGCTTGTTTCGTGTTCCCA
GGTGATTAAGGTAGTCATTGTGTTTTCCTCGTTTCGTTTCGATGTGGTAATTATGCCAAA
CTACCCCAATCGAGTTTTAACAAAAAGTGCTATTTGCCAATCAGGATGTAATCCGTCATT
TCAGCATTGACACGGCAAAAGCAAGGATTGAAAAAGCAATTGACAACAGCGAAAATCCAG
CAGCAGCGATAAAGAACACCACGGCATGATCTAAAAGTTTCTTATTCATCAAACGCCCCC
AACAATATCAAACCAAGAACAACAAGCGCCACGCCAGGATGACCGATTGCAACGCACCAC
ATTGCAGCGACAAATGCGAATGTTTTCATTGAAGTCTCCCGATAGCGTCAGCAAAAACCA
GGAATGCAAGCGATCCGAAAGTTGCCGCAATCGCAAGGATAAGCAAGCATAAACATAACT
CAATATATTTCGTATTCATTAGCGGATATCCTCAAACGTTTTGAAGTATAAGCGGCCTTT
TGCGTTGTGCCATAGCAATTCATTTGTTTCTATCCATCTTCCGGTGTCTGTGCGGATTAT
GCGACCGGAACGATATTCCAGCTTTACTATGTTGCCGCCACTTCTTAGATATAGGCGATT
AACAATTGCAGTTGTACACATCCTGGATTCTTTCAGCAGGTCTACACCATTAAAACGGAT
CGGACAAAACATGATTATTCCCCTTGCTCAACCATTGACTTAATCAGATCCTGATAACGC
TTAATTCCGATTGCCGCGCCCTGGAGTTGTTCACCTTCCAGATCCGTGATATTTGGCATT
GTGTTTAGCAGTCGCTTGGCGATTCGCTCACCGATCCTCTTTTCCAGTGCCGGAATATCA
TTTTTTGTGAATACCTGTCGCTTTGCCTCGCGGTTGATTGCCACCTTAAACCCTGGCGCT
TCCTCAACCGGATCAGTGCGAGTAATTGAAACCGTTCCGATCTCCTTCACGTCAGGGACG
CGATTCATGATCGTGCGAATGTATGAGGCGTTAACCTTCTTCCTGTCTTGCTCGTCGAGA
TTGTCCTCGATAAGAGCATCGGCAATGATGTAATTATCAAGCTCTCCGCTTGCTAACTTC
TTCGCCAGTTCACGCAAGCTGTCGCAAAACTCTTTGGTAGTTGCCATGATTGGTTATCCT
CCTGTTGGTTAGTTCGTGGCGGCATATTATCGCCGCCATTTGTTCTCGTCAAGTAGTTTT
TTGTGTGCTTACGTTCCATACCTATAGTGGAACAAATTTTCAGCGTAAGTTAATGTTTTA
CTTGAAATTGAAAACGCGTTCTTTGGCTGTTCATGCGTTCATGAATTTTCGCGGATTCCT
ATATATAAATATATTTTTCTCAATCCACCACTAGACCAGGCGCAATAGGCTAAATATTAA
CCAAATACCACCATCAACAATATATCACCCACCACTAACCATATGGAACATTAGAAACAG
TATAGAAACAAATAAATACATATATATAGATAACTTACTGTTTTAATTATTATTATATAG
AGTTTTATTTTGTTCTTTTTGTGTGGCTAACACATAGAACCGTCGGAGAACTACGCCGGG
TAGATTTGTGAACACCCATGAACGGAAGATAACAGCGCGGAGCCTGGAGTGATGTTTTGC
GGGAAAAGTTTTCTCTGATCGGCAAGGTGTGATTATTTCTGCTCTCTCACTACTCAAATC
TAATCAAATCAAATCAATTTCAATCACAAAATCTCACACATCAATCATCATCTGATCATA
TAACGATCACATAACAGGATCATGAGCCTGGTCGATACGTCGAACTGATTGTTCGTTAAG
ATGAACGTTGGTAATTTTGCTGATACTGCATGAATAGCACTTTTTGTTAAAACCAATCAG
TCACGAATTGCTATGATTACCTCAACGAAACGAACTGAGGATTACACGATGGAAAACTTA
GTGATTGCAGTAGACAAGACAAACTTTGTACTCGCAGCGGCTGACAAAACAAACTTTTGG
ATCGTGTGGGACAAAATCGACGCAGCAATCCCAACTGGCAACAAATACCTGAACAATCTG
GAAATGCACTATTGCGAAGCGCTAAACATGCAGACGGAAGGTAAAGCGGCTTATGAGATC
GCTAAACATTTCGGCTTTGAAACTCTTGAGCAAATGTTCACGGCGGTTGCTAATCAGGCA
GAAGAAGATTTCGAAATTAAAGCGTTTGGCTAATAGCACGAATTGCTAAAAAAGGATCGG
GGTATCTTGATATAGTTGCCCCATCGAAACCAATCAGGAGATTAACGATGGAAATTCAAA
CTCAATTCGAAACTGTGGTTACTGTACGCGATGCGAGCCACGAAAAAGGATTGTTCAAAA
AAGGTACTGAGGTTACTGTTGATATTGATGATATGGTTGCATATCACGGCGGCATTACCT
GGAATATTCAGGTTGTTGAGCCAGGCAAGAAATTTAAACTTCTGGCATTCAATACCGTAT
TTGAAACTGTGGAGGCTGCACAATGAGTCGTTTAAATTTCATCGGAAAGAAAGTAAAAGT
TAAATCATCTGGAAAGTGCATGTTAAGAGTTTGGCGCTTTGATGAAGATCGCGAATATGA
AATTTTGATGGATAGACAAAATGACTTGCATATTTCAGACAATCGCGGCCTTGCCCTTTG
TCTTTGCATAACTGAAGATGGAAAGCGGTTATACGCGCCAGGCGTTGAAAGTATTTATTC
AATTGAGGTGATGAAATGATCAATAATACTTATCGCGTCGTTGCAATCAGTCGCGCAAAT
GGTAAGCGAGTTGTGTGCTATGAAGGTGATCGCGCTTGCCTGGCAACTGATACATATGAG
GAATTAACAAAACGTCGCCCTACTTTTTACTCCGATTTCAAAGTGGTGCTTGAGCGCCTT
GAGCCTGTAATTGTCATGGAGAGTGATTGATGGAGCTATTTTTTATTCCTAACGCTTGGA
TTCTTGCTGTTGCTAATGACCATTACGGAGGCGACGGAAAGCGTGCGAAAAGGTATTCGT
ATTTTAATTAAATAGCACTTTTTGTTAAAAGCGATCCGGTTTGTTTTGGCATAATACATT
CATCGAAACGAACCGGAGAATTTAAAATGAATTGGCACGACTTTTTTATATATGATTGCG
GTAATCTTATAAGAAAGCCGCGCACAAACATAAGATCATATAGAGATTCTGCATGGAATA
AAAAGTGGGCTGGAAAAGTTGCAGGAAGCATTAGAAATGATGGATACATAGAGATAACAT
TGAATGATAAAAGTTATCTTGCGCATCGAATCGTTTGGGAAATGCATAATGGCAAAATTC
CAGAAGGAATGCAGATTGATCATATAAACCACGACAGAATTGATAACAGGATAGAAAACC
TACGCCTTGTTAGTGCAAATGAAAACGGTAAAAATCAATCATATTGCAAAGCAAATAAAT
CAGGGTTTAACGGTGTTTCGTGGCATTCCGTAGCTAAAAAATGGAGAGCAAGATTAAAAG
TAGAAGGAAAAGAAATCCATATTGGTCTTTTTGATTCTCCTGAATTAGCTAGCATGGCTA
TTGAGAGAGAAAAAATAAACAGGGGTTTTCATGAAAACCACGGCAAAAAGAAAGCGTAGG
CAAATTAATTGCAGGTGTTCCGCTTATCATTTCGTTCATAGGTTTGGCGGCGGCTTGTGT
ACCGGAATTCAGATCGCTGAGGAAAACGTAGGCGGCAACCTTTGCCAGCACTGCTATCTG
TTTAATAGTGGCTGTGAAGTGCTAAAAGGCCAAGAGTCGCCGCGCGAATGCGCATATGTG
CAAGAATTCATAGAATATCATGAGGTTAAATTATGACCTACTTCAAAAATATAAAAACCG
GAAGAATTTACCGCGTGAACCTGCGTCATAAAAACGCTTTAATTATGAGCAAAGGAGGCT
GTTGGGTTAGGTCTGCACGTTATAGAAATGCAGATTTAACAGGCTATCCGTTTATCGCCG
TAAACAGCACGAATTGCTAAAAACGATATCGCTCTGACTGGTATCATACTTTCACACAAA
CAAAATGGAGAATCAAAAATGAAACTGAAAACTGAATCCATCATGAACATTGTCAAAGCT
CACGGGAAAGTAGTTTTGAAAATGGATCGCGATTCTGGATTCCATCAGTTAACGATTACC
CGCGTAAAAACTGGTTACGCTGTTGGCGAACACCCAGGCGGAAGAATACGTCGCATGACT
GAAAGCGATGCTTACGCGATGATTGACGAAATGAGCATGTTTATTGAGAAGTGGGGTTGA
TATGGTTCAAGATGCTTTCTTTGCTCGCCTACATGAAGCGGAGGCGGCTGGACTCAACAA
GGAATGGGCGCTTAAAGTTGCCTATCTTGAAATAACGCTTGATGACGCATTAGGCGCTAT
GGATATGGATCAAGAAAGTAGCGTGATGCTTGCTGATCCTAACGCAATAATCAATGATTG
CGGTTGTAACTTCGATCCATCTTGCAAGCGTTGTTTCCCATTCTAAGGAGTATTTAAAAT
GACACCTATCGAAAAAATTTGCTGTCCAAAACACGGCGGAAGCGGGGATAAAACAACATG
CCCATTCTGTAAGTAAAAAATAGAGGCCTGTTTCGCATTATGTCGCGATTTCAGGCCGTA
ACTAATACGATTGTCACACCTACAAACAAAATGCCTTGCAAGGTAATACAGAAAGGATTT
AAACATGCTTATTTTCTCACTATTTGATGGTTCTGGTTATGCTGCGTTGCCGTGGGCTGA
ACGTGGTCACAAAGTGCTGTGCTTTAATTTCGACGGTGCTGATCATGGTGATTACCAATC
AGTGCGCGTGACTCATCCCAACATAGTTTATTTGAATGAGTGGATCGGATTATCATTCCA
GCATAACGCAATGATGCGCGTCTTTGGCACTCCAGATTTTATTATTGCATTCCCTCCATG
CACTGATTTAGCTGTTAGCGGATCGCGTCACTTTGCAGCAAAGCGTGAACGTGATCCAGA
CTTCCAGAAAAAGGCGGTTGAAACGGCAAAGATTGCTGCCAATATTGCGGATCGGTTTAA
CGTTCCATACATGATTGAGAATCCTGTTAGTGTTCTGTCGTCAATGTGGCGGCAACCTGA
TTTTATCTTTCATCCTTGCGCGTTTGGAGGTCTGTTGCCGGAAGATGATAAACATCCTCA
CTTTCCTGAAATCATTCCCGCTCGCGATGCGTACACCAAAAAAACTTGTCTTTGGACGGG
TAACGGCTTTGTAATGCCGGATACAAACGCAGTATTGCAAACAGGGAACAACAATCCAGG
CTGGCAAAAGCTCGGCGGCAAGTCTAAGCGAACAAAGCTGATCCGCTCACTCACTCCGCG
CGGCTTTGCGCTTGCTGTATGCCTGGCAAATCAGAAATAGCACTTTTTGTTAAAACGCCG
GATCACGTATCCGGCATAATGCTTTCAACGAAAACAAAAGGAGATAAATCAAATGGCTAA
ATTCAAATCAATCAAACTTATCGGATCTGATAACCGCCTATACAAAGCGCGGGTAGACGA
AAATGATGATACTTTCGTTAAGTTAGATGGGAAATGGATTCACGCCAACTTAACCGGATT
AACATTCCGAAATGTTAAGACCAAAACCATTAAATGCACATGGGTAAACCACTCCATGCA
GATCAAGAAAACTTTCAAAGCCGATAAGCGTTACCAGATCGAACAAGGCCGCGTGTTAGG
TGCTGTTGCTGGATACGTGTTCGACGAAAACGGCGACCGTTTCACGCTGTATCGTGAAGA
AGTTGGATTTTCAGCCGCTGGCGGTGCTTATCTGTTTGAGGCCAAATACTCATGAGTTTA
CCAGAAAAAGACTATACGCCTCACCGTTCTGAGGCTATGCAAAAATATCGTTACAAGGTT
GTTTTTAGTGCGATATCGTCATTAAAGGAAGGTGAAACGGTCTATCGCATCAGGGATAAG
CATTCATATTTGCTGATTGGTCATACGTTCCATCGTTCGCTTGCATTCGATCCTAAAACT
CTTGAATGTCATAGCATTATAGATGGAAGGTTATTAGCCAGGGTTGAGCCGATCAAATAG
CACGAATTGCTAAAAGGCTGTAGAATGTTGCCGTTATAATTAGCGGCAACAAGAAGGAGA
TTTTTAGATGTTTGACTTTGACACCAACAAATTGACACCTCAACAAGTTATAGCCATTGC
AGAATCGCAGGGAACATCGCCGCTTCGCGTTGCTATTCAGTCTAACGGCTACCGCCAATC
GTCCAGCTTTTGGGAACCTGTAAAGGATATCAACGGCGCTAATGACCGCTATCCTGTGAT
CTCTTTGGGTAATGATGTTGATGTTGTGGGTAAACTGTCACGTAGCATTGCGCAATCTGT
TCAATTTCCTGAATCATCGGCCTATATGCATTTCATTGGTTGCGTTTCCGCCGCTATGGT
TGGACGCTTCCAAGTTGAGTACCACGGCACTGAACAACCAACGGCGCTTTATGTTGTAAC
CAGTCAGCCGCCATCAACTGGTAAATCAGCAATCAACAGCCTGGCTATTGCGCCAATGGT
CTGTGAAGTTGAGCGCATCAACGAACAACGCAAGAAAGAGCGAAAGAAGATCGCAGCGAA
ATTAAAAGGCGTTGAAAAGGAATTGAAAGCTGAACGTTCCGGCACTGAGTTAGCGGCGCT
GTTTGAGGAAAAGGAGGAATTAGAGGAAAAGTTAGAAAAAATGTGCGATATCGTTTTTCC
GGTATCAGACACCACGCCGGAAGGGTTAGCAAAGATCAACAATCGCCAGGGTAACTTTGC
TGTCATTTCCGATGAAGCAACAAGCATCAACAGCTTACTCGGATTGACTTACGCTAATAG
TGATCGCAAGACTAACAGCGAATTGGTTCTAAAAGCATGGGACGCCGGACACGTTTCAAT
AGCTCGCGCCAACTCTGAAAACAATATGAGTTTTGTTGCGTTGGGCTGCATGAGCGTAAT
TGCCCAGGATGAAACGATAAAAGGGATCATGGATGCAGGGGCGCGTGGTATCGGTGTTAG
TGAACGTTTCTTGCTTGTACGTGAAAAAACAATGTTAGGCGAGCGTGTATTTATTGATGA
AAATGGTGATCTTGATTACACGCCAGTTGATAAAGAATTAAAGGCGCAATATTTCCAGTT
GATTCATAACATCATGACTGAACAAAATGTGGTATTGAAGCCGTCACGTTCTGCAATGCG
TGTTCTTAATATTGCTCGCCAGCAATTAGAGCCAGATTTAGCAGACGGCGGGAAATATTC
ACATACAATGTTGCGTGGTGCTTTGGGTAAAATGGATAAGCAAGTGATCCGCATTGCGTC
AGTATTGCACACGATCCGCAATTGGTTTAATCCAAACGGTACGCCGCAAAAGTCGCGAGA
AATTGAAGTTGAGACAATGCAAGAGGCTTTGGTGATGTTCAGCGAATTAAGCAAAACATA
TATCAACGCCGCGAATGCTTCCGGTCACGCTGGAGATGATGCAGAAATGAGCAAGTTAAT
TGAAATAATCATTAAGATCTCACGTCAAAATAAAGGCGTTACCAATGCAAGAGCTATCTA
TGAAAGTGCGCGTAAGGTTAAGCCGTTTACAGGTCAATCAGGAGTGATGAAACGAATTGA
GGAACAATTATTGCCAATGCTTGAAGAAAAGAATTATGTTTGCAGCGTTGGTAAATTTGT
TTATGTAAATCCGGCGTTAATGGGGTAATCATGTTCTTATTAGATCTGTATCGCTTTTGC
GAGTCTCGCGAGAAATTCAACCGCCAAGAGTTAGCAAGATTTATATTCCGTCACCGTGAA
TGTGAAAGGCTCGCCAAAAATGCGGGGGTAACGCCCCGCTATTTCGCTTCAAGCTCAAGC
AAAGAGTTTATAGCGCGAATGATGGGTTATGGATATCTTGACGGCGTTCATTGCGTGTAC
TGGTGCAAAAACAAGATGAAAAGGCCGTTTAATTTTGAGTTGCATAGCCTGGAAGGTGAT
TCCAATCGTTATGTGTGGGAGATGATGAACATTGAGAAAATGAGCGATGAAGAATTATTC
AGCAAGCCAGCATTTGATAGATCTTATTTTGAAAGAAAGTTCAGCAATGCAAATTTCGCA
TGATGATTTTGTCTATGCACTAATGTTGTTGCTTTACAGTGATATGCGAGCGTTTGAAGA
TAATGAGCACCAGTTAACAAATGATGACGGTGAACTACTAATTCACGTCAGGCGCTTTGA
ATAGCACTTTTTGTTAAAACACCCGATCTTGTATCGGGTATATTTGTTTTAACGAAACGT
AGCAACAAAAGGAAAACAAAATGTTAAAATGCACTATCAAAACAATTCGCGTTGTTAATG
TTACATTTGACGTTTTCAACAACAAAGGAACCGCATATGTTGATGATGGTAGTACGTTTG
GCATTAACCGCTGCGAATATGAAAAAATGCTGTGGGATATAAAAAAGAAAGGCGCTCACA
TTATTGAGGAAAAGGATTATAGCGGTAATAGTTTCCAAGACTTAATGAACAATATTTTTT
AAAAGAAAAGGCGGCTTATGCCGCCTTGTTACGTTTAAGTGCCCACCCTGCCAGCATACC
CGCAATAAACATCAATATACCCATTGCGATTGCCGGAACATTAAGACCATCATTATTTCG
GATCTCTATTCTTTCCGCTGTGATGTTATCTGCCTTAATGCTTGAAGTGCTCACCTGTTT
TTTATTCGACGTGTCAACCTTTCCAACAGCCGATTCTTTAAATGTGGTTTCCTGCTTGCT
TGATGTATCATGCTTTGCAGTTACACCAACAGTTTGTTTCACATTTTCAGATCCTGCCTG
TGCTGTTATATCGGGCTTGCTACCAATCAGGCCAGGTAACAAGCTCGACGCAGAACAACC
GGAAAGCATAACGCACATCAAAACAGCCATTAATTTCTTTTTCATTTAAGATCCCTTACG
CAATATTTATATTCGACGGCGCGGCGGTTCTTCAATCCTCTTGATTTTTCCTTTTTACCA
GTTTTAGGGTTCCGGTAATATGTCCAGTTCCATAGCTCACCACAACCGCCCCATAGATCA
CCATTGTTGATCTTTTTCAGCATGGTTGACTTACGAAACGCGCCAGTACCAGCATTAAAA
GTGAATGAGTATAGAGCCGCTCGCATTGATAAAGGAATTTCAACCTTAACCGCCTTATCA
ACAGCGTCAGCCGCTACTTTCATGTGTTTTGCTAATAGCGCGTCACATTCTCGCCTGGTA
TATTCCTTTCCAAGAATCACATCCTTTCCGGTAATGCCGTGGCATACCGTCCAGATTCCA
GCAATATCCTTGTAAGGCTTATACTTTACGCCCTCAATTTCTTCAATCAAAGGCGATGCC
AACATTAAAGCCGCCCCTGTGACGGTGGCGGCTATACCTTTCTTGATTGTCATTACACCC
TCTCTTTCTGCCGTAGATTAAGCGCCGTTTTTAAATCCCCTGAATCTAAAGCCTCTCGGA
TCGCTTTACTATCACGCCATTTCAGCCATGCGCCCCAACAGCCGAAAATAGCGAACAAAA
TAAACGTAGCAAGTGCAAGCAAAAACTCACCAGTAAAAGATCCAACTACCGTTGCACCTC
CGAAAGAGTTTTGCGCCATTAAAAGAAAGTCTCTCATAATCTGATCCTCAATTAAATTAA
AATGTCAATGCCAAAATTAGTGTGAATATGTTAACCACAAAAACAAAAAAAGGCCAGCAC
TTAGCCAGCCTTTTTCTTGATAATTTAATTAAGCAATCATTTCAGGGTAAAACATAAACC
GCCCTATTTCGCCATATTCCTTATCATAAATAATTACCGCCGCCCTGCGACGTGAACGCC
AGCCACCACGCGCGGCGTAAGCGTCTTTTGCTGCCATAGTGCTATGCACCTCAACAATGC
CAAGGCTCGTTTCTGTAACAGTCTGGTGATGCCAATGCCCTACATGCGCATACATTGAAT
TGGAGTTTCCGAAATCTTCCCGCCAATCCGCTGCACACATCATTAAAAGCGTTTCTGGCT
TGCGTACAGTGTGACCATGATGATAAGCCAGGAAAGTTTTACCATATTGAGTGTGATGCA
CGACGCGCGGAGAAACATCAACCTTTACGCGTGGCTCATTCTCATACGCCGCCGCCATTG
CAGCACGTAGCCAGATCATTCCTGATTGGTCGTGATTACCTTCAATGATCTGCAATTCAA
CCTCTTTATGTTTTTCCAGCATTCTGCTTACTGCTCTGCGCGTGGCTCGAATGGCAACAT
AAACAAGTTTCGCATATCGCGAATCCTGATCCAAAACGTGACCGCTTGCAGGTGTTACAG
CCTCCAGGCCGTCGCTGTGAATCATATCACCACCAATCAGCAATACAGCCTTTTCGCTAT
TCGGTGCTTTATCGACGGCATAATCAAAGAATTCATTCATGATCTTTTCTGCCGTTGCTG
TGTCGTAATTCTCTCCGCTTTCATGCTTATGAGCCATTGCACCAATATGAATATCAAAGA
TCGGATATAGCGCAAGGGTTTTCGAATAGTCAGTAATCTTTTCCGGCTGTGGTTCCAGGC
GTGGCAACTCGTCAGAAAATGCAGCCATAGCCGCTTGCATTATTGCCTCTAACTGGTCTT
TATCTTTGTGCGTCTTAATCCAGGTTAGTTTAGTATTGCCGTTTTTATCAACTAACTTTG
ACTCGCCAATTACCGCAAAGCCAGGTGATCCGATAGTTGAGATCTCGCCACGCTTTGCTA
ATTTGCTGTGGCGGTGCTCAACCTGGCGCAAGCTCATTCCGTATTTTTCCGCAATCTCAC
GCAGTTTTAAACCCGCCTCACGTTCAGCGATCAATTGCTCGTCGGTAATCTTTTTTAAAC
TCATTTTAACATCCTCTTAATTCATAACAACGGCGTACATAATAACCCAAGCGATAACAG
GCGGCAACAAAATAACAGAAATGTATCTCATTTGTAATATTTCTTCTGCTCTTTGGTTTT
GTTAATAACTTCTTTATAGTCGATTGTTGTTGGTATTGCTGGAACACGGTGAACACGTCG
CGGCTTTTCTTCAACATACGCCATTTCGTCACCAACAATAACGCACACGTCTTTAATATC
GAAATACTTTGCTATTTTGGCGATCCCTTCATCAAGACCGCCTTTTTTAGCAAGCTCCCA
AGTTTCCTTTTTACCTTTCTTCACTTTCATAACCGTAAGCCTTTTTAAGTAACTCCATTG
CGTCATTCCAAAGTATTTGTGCAATAACCTTTGGATTTTTGCGATCAAGCATAAACAATG
ATTCTTTAGCCAGCCATTGCGCATCTTTGAAGTATTCGACGTTTACGATTTTCATTTTTG
AAATCCTCTTGTTTTCCGTTAAGCACATAATACAAAACCCGCCGTAGCGGGTTTTAACAA
AAAGTGCTATTTCACATTGAAAAATCGTGCGCCCTCTCTGCGTTTGTATTGTATCAGGTC
GTGCTCAATTTTTCTTTCCCACATGTACAAAGGCGCAACTTTGATTTCGTCAAATGTCAT
ATCCAGGCAGTTTATCAACTCCATTTTATCATCGTTATAACCATGCTTATCAAGTAAGCG
AATTGTCATACCGACAAATAAATCCCATTTCATGTGATCGGCTAATCGAATGCTCATTTT
AAATACTCCGCGCTTTGGCTCGTTTATTTTTTACGGAAGGGCAAATGTCGCTAATCTTCA
CATAGTGCGTTTGCTGTTGCTCACCTTCTTCCAATTGGCGCATGATAAAAATTACACTAC
CTTTATTGTTTCCGCTAACTGGCTCACCTGTCATGCCAGAAATAAACGCCACTCGACCAG
TTCTGGAATATTCGCGACCTTCTTCATCCTTCCAGGTTTCACCAGTAATCCAGATAATTT
CTGCCGCATGTTTTTGTGCGTCACTAAACCATGCAGTTGAATTATCAGCCGGAAGTAAAA
TATCAATCTGGTTTCCGTGTTCCATCTGCTCAATTGCTTTTTTGATAAATGGATCTGGAT
TGCTGTATGGTGGATTTAACCAAACGTGCTTATTGCTACCCCACCAACGCTTTAAGCAGT
TTGTTTCTTTGCTGTAAAATTTATCGCATTTTTTATTATCTTCCGTTGCTGCTGCGTCAA
GATCATATTTGCCGTAACGTTCAGCAAGGTATTCAACAATTTCATCTGGAGTTTCCCACA
AGTCGCGCACTACGTCCGGCGTTTTACTTCCTGCATAACGGTTGCCGCTAACCTTAAATA
ATGGGTTTTCTCTAACTGGCTGATAATGACCGCCTGAACGTAAAGCAATATCTGAATATC
TGCCGTGTTGCAGTTCATGAAAATCCATTTCCGGCTCAAAAGTATCAATGGTTGCTTTGT
CCTGAAAATCCTTAGCCATTTTATTTTACCTCGCAAGTTAGAGTGTTCTGATAAACGCCA
ACTTTAATATCAATGGTGCTATCGTTAACAGTGTAAAAAGCCGTTCCAATTGGCGTAAAC
AGCTTATATTGATTTTTGCTGATATTATCCATTACAACGCCTGTGCTTTTACCTTGCACA
ACTTTCATATATTCGCTTGTAACCTCAAACGCTTCATTTCCGCAAATATATGTTTTTGGT
TTATCCATGCAACCAACAATGGCAAGTGATAAAGCAACTACTGATAACATCTTTTTCATG
GTTAATTCTCCGTTTCGTTCTGATGTGTGCATTATGGCGCATTCCGTCGCGCCTGTTTTA
ACAAAAAGTGCTATTTTGTAGCGTCTGCGATAGCCAATTTTACTTGATCTACACCATAGC
ATACTGCGGCAAATCCTCCGCATTCCCTAACGCGGCGCAAGAATTCCCTTTGTTCCTGGC
TTACTTTAGTTCCTTTCTTGTTGGCTCGTTTAAGCTCAATTGCAATGAATGCATATTTGC
AGCCAATAAAACCAGTCAGGATTAGAATGTCGCTTACTCCTTTTTTTACACCTGCTTGCT
GATCCTTTACTGCTGTGCCTATTGTTTTCTTCCCTTCGTTTTTAGTGTGCCAAACAAGCC
ACATTGGAAACTCATATGTTAACCATGAATAAGAATCAACCTGGTGCGCTTCCTCTTTTC
TGTCATCTGGATCTGATTTGTCGTAATACTCCAGATAATCGCCTTTATCAGTAACCATTT
TTAAAACTCCTTCTGTGCGATCAAATCTTCTTTCTTTGTGTTCTTGCGATGGGTAACACG
TTTTGGCGGCAATATTTGCCCCGCATAACTCATGATCTTAATTGCATTGCGGCAAGCGAT
CATAAAGTTTGCGGCTTGGCGATCTATAACATGTGTTCTAACCGCCTTTGTCCAAAGCGT
TTTACATATCTTGTGTTCTGATTCAGGGAAATATTTTTCGTATGCCCTGAATTCTACGCC
GCCACTATCACGTAACGTGTAGCAGAATATTACGCCAGCCTGGTTTTTTGTCAGCGTCAC
ATTAAAATCTAACACGTCGCACCAATCGTTTTTACGGTACATCTTGCCGGATAAGTTATC
ATTTGGATCGCGTAACTGAACATTACAGCAACGGCATACACGCGCCGCAACGTCATTTTT
TGCGCCGCAACCTTTTACAATAACTTTCTTTGTGCGGTCATCAATCTGATCCTCACATTC
CTGGAACGTCCAGAAATGTTCACAACGATTTCCGTTTTCATCCTCATTAATGCAACGGCG
AGCATAGAATGAATTTTCTGTACCGCAAATCGGGCAATATTTCGGATCTTTGTCCTGTGA
TTTACGTAACTGGAATTGCGCTTGCTCTAACATCGGATCGAAATACAAATTACCCATTTC
AAACATCGTGCCTGAAAAATCCAAAACAAGGTGATCCGTTTTGTGCATCCCCAATACTTC
TTTATGGTATGTTTTTAATTGACGCATACCACGCCCCAAAAGCTGAGTTAATAGCGTGAG
ACTTCCGATCTTACGCAAAATAACAGAAGTATCCCACGGCGGCACGTTCACGCCAGTTGT
TAGCGCCTGAACCTGAAAAATATATTTCACCCTGCCTTCATAAGCATCCTCAAGCCATAA
GCGCCTCTGTTTCTCTCCAGTTTTTCCGGTAATAATGTGATATTTCGTGCCAGGAGGAAG
CGCCGCCGCTGCCTCTTTGCAGTGCCTTTCGTCAGCGCAAGTAACAAGAGCGCACAAGCG
GTTTTTCATCACCTCGTGAACCATCTGCATTATTTTCTGCGTCATGGTTCCTGATTGGTG
AATCTTGTCACTCATGGCCTTTAATTGCTTCTGGTCATAATCAGCAACGCCAAATTCATT
CACTGGCGTAAATTCTGATAAGTCGTAACCCAAATCACCAACATTGCCAAAATAAGTGGG
AACAACTGAACCAAATTTTACCAGGTAATCAGTATCAATGTTTGTGATCTGCTCACGCCA
AAACCCAGGAATTCTTTTATCTTCGACAAGTATCGGCTCAATTCCGCGATATTCCGATCC
TGTCATTCCAAATATACGCAACTGATGACCGTAAGTCTCAAGACAACGGCGGCGCATTTC
TGCAATGATAATGGTGTATTGTGCGCGGCCTGAACCAATCAGCAACGGATTGCCTTCAAC
TGGTTCACCTTTCGGTGTCATCATTTGCTCTATGCTTTCGCCGTTTTCCTCGGCTTCAAC
AATATCATTCCAGTTGACTTGATGACATTCATCTATACCTAAAACCTGTGGGACGTAATC
ACCTAACGCCTTATTCAATCCGCCAATTACAGTACCTTCCGATCCTACAACAATCGGGAA
ATAAGCGCTTTTGGTATTCAGGCCAGCGCAATAAACGGAATTCGGAACACCAAAGGCGCT
AATCTCTTTTGAATCCTGATCCACAATTTCAGATTGACGCGCCAGAAATAACGCTTTCAA
TCCCATTTTCCTGCACTGAGCCGCAACCATAGCAAAGATGATTGTTTTACCTGCCGACAC
CGCCGCTTTAACGAAAAACGGATCAACATACTTACTCAAGCGCTTTGCAATTTCAGCGTA
AGCAACGCATTGGTATTCATACGGGACAATCTCACCAACCGTGAAACGCTTCTGGATTGC
CTTAATCCTTTCCTCTCCCAGATCTGCAATCTGCTTTTCAATGTTAGGAATCATTTTTTA
TCCTTTTGACTAAATGCCATTTGCGGCATATCATACACAAACTTAATCAAGTTTTTTAGC
AAAAAGTGCTATAGGTGAAAATATGAGCGAAATCAAAATTGCGGCCTCAACGGGCGAAAT
CGACAAACGCACAATCAATGGTAACAACGGCGTAAGGCGTGGTAAAGACCGGAAGCGCCG
CGCACCTGTTAGCGGTTATCTGGTACTAAAGGAAGAAGTAAGGGCAGGATTAAAAGCGAG
GCTTGATGAGGTGGTTAGCGCTTATGGCGGCTGTGCTGCGTTGGCGCGTGAGATTGGCGT
TAACCTTAAAACAGTGTGGGGATGGAAAGAGCGCGGCATGATTTCAGCTAAAGGCGCTGA
ACTGATCCAAAAGGATTACCGCCGCAAAGGTTTTAACGGCTTCCGCGCTTCATATTGCCG
TCCAGATTTGCGCTTTGACAACAACGGCAAGCCATTAGAGAATCGTTGCAGCAACAGAAA
GCTCATGCGATTTGTAACCAAAGAGGAAGCGGAAGCAAGAGGATTTGTGCGTCCTTACGA
TCCGTTGCGGTCTATGAGTCCAGAAGAACGCGAGAAGGAGAAAGCGCGGCGCAAGGCTGA
ACGAGACAAGGCGAAAGAAGAACGGCGCAAAGCAAAGGAAGCCAATCAGAAGAAACGCGT
TGTGATTTCCGTCGATAGCACTGATTGCTAAAAATTCCGGCCTGGCATGGTTTATCATCG
TGCCAGTTTTTTTTATTTGGATGTTGAGAAATGCATTATCAAAAAGAAGAAGTAATCCCA
AAGATGCGCGGCATGTGGCAAGCGGCATTGATGAAACTTTGCGATATTCCTCAATTAACC
TTTAACGGAAAGCATCAGCCTTGCCCTTATTGCGGCGGTAAAGATCGCTTTCGTTGGACT
GACAAAATCAACGAGAAAGGCGACGGCGGAAACATTTGCAACCAATGCGGGAATGATTCA
GGGATCGGATTATTCATGCGCATACGTGGTGAAAATTACAGTGAAGCAATCGACACTCTA
GGAGAATGGTTAAACCTGGTTCCGGTTGAGGTTGTCAACAAGGCCAACAAAGCGGCAAAA
CGTGACTCAGGTTACAATTTCGGATCGCAAGCACCACATGAAAAATGTGTTGAGGTGATG
GATAAAACTGAACGCATGGAAATAACAAATCTAAGCGTTTTTGAGGCGTTTTATCCATTA
GACGGCGAATCGTATCAAGTTGGAGTAAAAACTCATGACAATGGATATTGCGAGCATATA
CACGCAATTCCTTGCCACTTGGTGCATAGCGATGAACTGGATGATGAAATGTGTAATATT
CTTTTCGTCAATGAGGAAGGTCAAAGCAGTTTTTACGCTAAGGATTACACGCGCGGCTCC
GTTGCGGTAACTGGCAAGACTGACAAAACAATTTACCTTTGCGTTGATTGGATTGATGCG
CAACATATCCACCTAGCTACGGGACAAGAAGTATGGGCTTGCTTCTCTGCGTCGAATCTT
GAAATAGTAGCGTATCGCTACAAAGGTGATCGCAAAATGCGCGTTGTGTGCCATGCTAAT
GATCGCGATACTTTGATTGCTGCTGATGATCGCGAATTAGACGTAATGTTACCAATTAAC
GGTAAATTCAAAAATGGAATAGAGCGGAGGCTTTACAAGGCAAGTGACTTATTATAATAT
TCATCCTGCTACTAACACTTTTCGATGAATCTGTTTCATTGACTTCTCGTTGTCGTTAAC
GATGCCCCGCTTCGGCGGGGTTTTTTATTGCATGTAACAAATGGCTGTAGCATAATTACA
TGACCTATTTTAACAAAAAATGCTATGAGGTGAAATTATGGCTTTATATCGCACTGGTAC
GGCTGCAATGGACGCACAAGGCGTTATCACTGGTACTGGCACAAAATGGCGTGAGCCGCT
TTCTCTTATTCGCACTGGCGCAACAATCGTTTTTTTAACCACGCCTTTAAAGCTGGCGGT
TATCAGCGATATTGTAAGCGACACGGAAATGAAAGCGATCCAGACTGACGGCGAGCCAGT
AGCAAACGGAAATTATGTAATCCTGTTGAATGACTCGCTAACTGTCGATGGAATGGCGCA
AGATGTAGCGGAAACGCTGAGATATTACCAAAGTAAAGAAACGGTTATTGAGGAAGCAAT
TGAATTCTTCAAAAACTTTGACCTTAAAACATTACAGGATCTTGTTGCTCGCGCTGAGTC
TTCCGCACAAAAAACGGATGCAGATCGCGCCGCAACTGAGCAATTAAAAAACGACACGCA
AGCTATTAAGGATGCCGCTGTTTCTGAGACTACGCAAATTAAAGATACTGCGATTGCAGA
AACTCAACGGATTAAGGATGCTGCTGTTTATGAAACCAATCAGATTAAGGCAGACACTGA
CGCAATAAAAACGCAGACGCAACAAATAAAAGATAGCGCCGTTGCTGAAATTGGATCAAT
TAAAAACGAGGCTGTTAATGCTCGCGATGCAGCAAAGGAATCACAACTTGCCGCTGAACA
ATCAAAGATTGCCGCCGACTCTGCAAAGGCTGGAGCTGAAACTGCGCGTGATGAAGCTCG
CCAATGGGCACAACAAGTTAACCCTGAAAACCTTCTGCATAAGGATCAGAATCTTAATGA
TCTTGCTAATAAAGATTTAGCAAGGGAGGCGCTGAAAGTTGAGGCTGTTAATTCAGTTAA
GGGGCAAAATCCAGGTGATTATAACTCATTTCGTAACCCTGCATGGACTTATGAATTACG
GATCGCAAACAACGGCGAGTGGCGAGTTGCAAGAAATAGCGATAACAGCACATCCGCTCT
TTCTGTTGGTGCTGGCGGCACTGGTGCGGAAAATATTGAAGGTGCAAGAAAAAACTTTCG
TGTGCAAGGTGTTTTTTGCATTACTAGCGAAACACCAGGAGATTTTAACTCCATAAAATC
ACCTGATGGAAAACTAAATATGCTTGTAGCTAACAATGGTGTGTGGGGTGTTCAAGATGA
TTATGGAAACATCAAACCATTACACATCGAACGAGGAGGCACTGGCGCTCAAAGCATAGG
GCAAGCAAGGAGTAATTTTGGCATTGGTGAAACTGACATTCCTGTTTTTAGAGGAATAAG
CCTAACAGAAAAAAACTCTGCAAACTCCGGCATACTTTATTTGTTAAACAAAAATGCAGA
AGGAGTACAGATTTCGTATTCAAGGGTTTACAACGAAATTCAGGGTGGGATTGCAAAGGC
AACTATTCAAGTAACTAGAGAAGGTGGCGACACAAACTATTATCAGTTTGATGAAAGTGG
CAACGCGCTGAATTACAACTCAATAACAATCGGTAGGGGGATTGGTAATGCTCTTGGTAG
TAACTCTATAGTAATTGGAGATACTGATACTGGATTTAGGCAAAATGGAGATGGCATTCT
TCAAGCTATTGCTGATAATCAAGTGATGTTTGCATTTACAAAGAGTGGCAATGTTGCATA
CAGAACTATTCAATCATTTGCTCCAGAAGATGCACGTTTTGCTTATGTTGAAGGTGTTAG
GAGGGGTGGTGCGAATTGCTTTATAGGTGGGCATGTTGAGGGTGGTGCTTTTACTGCATG
GCGTGATCGCGCCGCTGGTATGCTTGTTGAACTTCCAGGCGATGATGTAGCCGTTAACGT
TGTTAAGGTTGTAAGGTGGGGTTATGATTGGGTTTTTGGTATAGATGTTGCCAGATATGG
CGCTGGAGGTTGCGAAGCTCACCTAAACGTAAGAGGTACATCATACACTTTTAATGATGC
TGGTTATGCAAGCGCTGTTCAATGGGTTAATACATCTGATATTCGACTAAAGGCAAATCT
AAAGGAGATTAAAAGCGCCAAAGATAAAGTTAAATCAATTAAGGGATACACTTACTTTAA
GCGCAATAGCCTAGAAGAAGATGAAAATTCTTTCTACTCTGAGGAAGCTGGCGTAATAGC
TCAAGATGTTCAGACTGTTTTGCCGGAAGCGGTTTACAAGGTATCTGATTCTGAAATTTT
AGGCGTTAGCTATGGCGGTGTAA

Tolerable variations of the bacteriophage sequences will be known to those of skill in the art. For example, in some embodiments the bacteriophage comprises a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleic acid sequence set forth in any one SEQ ID NOs: 1-13.

### Methods

In another aspect, the invention includes a method of increasing antibiotic sensitivity and/or decreasing virulence in pathogenic bacteria. In some embodiments, the pathogenic bacteria are drug resistant. In some embodiments, the pathogenic bacteria are multi-drug resistant (MDR) bacteria. In some embodiments, the pathogenic bacteria are pan-drug resistant (PDR) bacteria.

The method comprises contacting the pathogenic bacteria with a lytic bacteriophage selected from the group consisting of OMKO1, LPS-5, TIVP-H6, LPS-TLTL, SFA1-1, SF60B, SFNHSI, SF37B, and U136B. In certain embodiments, the bacteriophage binds a molecule of an efflux pump, OmpA, OmpC, Type IV pilus, LPS or peptidoglycan in the bacteria and the bacteria either genetically resists bacteriophage infection or becomes infected and lysed by the bacteriophage. The genetically resistant bacteria have impaired efflux pumps and increased sensitivity to antibiotics, or have impaired virulence factors, such as reduced production of pyocyanin or elastase, that are responsible for decreased virulence.

In one embodiment, the bacteria are contacted with bacteriophage at a multiplicity of infection (MOI) of bacteriophage to bacteria in the range of about 0.0001 to about 10¹⁰. The MOI may range from about 0.0002 to about 10⁹, from about 0.0003 to about 10⁸, from about 0.0004 to about 10⁷, from about 0.0005 to about 10⁶, from about 0.0006 to about 10⁵, from about 0.0007 to about 10,000, from about 0.0008 to about 5,000, from about 0.0009 to about 2,500, from about 0.001 to about 1,000, from about 0.005 to about 500, from about 0.01 to about 100, from about 0.05 to about 50, from about 0.1 to about 10, or any range therebetween.

In another embodiment, the method further comprises contacting the genetically resistant bacteria with an antibiotic. The antibiotic includes any of the antibiotics described herein, any commonly known agent, and any combination thereof.

In another embodiment, the method comprises or further comprises contacting the genetically resistant bacteria with one or more antibiotics, e.g., 1-100 antibiotics or 1, 2, 3, 4, 5, 6, 7, 8, 9, 10, 11, 12, 13, 14, 15, 16, 17, 18, 19, 20, 21, 22, 23, 24, 25, or more antibiotics.

In yet another aspect, the invention includes a method of treating a bacterial infection in a subject in need thereof. In certain embodiments, the bacterial infection is drug resistant or multi-drug resistant (e.g. caused by a drug resistant or multi-drug resistant bacteria). The method comprises administering the pharmaceutical composition as described herein to the subject with the bacterial infection. In one embodiment, the composition is administered directly to a site of the bacterial infection. In another embodiment, the method further comprises administering an antibiotic as described herein to the subject. In one such embodiment, the antibiotic is co-administered with the pharmaceutical composition. In another such embodiment, the antibiotic is administered before or after the pharmaceutical composition is administered.

In some embodiments, the antibiotic can be administered minutes, hours, days, or weeks, before or after the pharmaceutical composition is administered, e.g.: 1, 5, 10, 15, 20, 30, or 45 minutes; 1, 2, 3, 4, 5, 6, 7, 8, 9, 10, 12, 14, 16, 18, 20, or 22 hours; 1, 2, 3, 4, 5, or 6 days; or 1 or 2 weeks, or any amount of time there between.

In another aspect, the invention includes a method of disrupting pathogenic bacteria associated with a biofilm and compositions for use thereof. In another aspect, the invention includes a method of preventing formation of a biofilm.

In some embodiments, the biofilm is on Dacron and/or any other prosthetic material.

In some embodiments, the pathogenic bacteria are associated with a biofilm. In some embodiments, the pathogenic bacterium is Pseudomonas aeruginosa. In some embodiments, the Pseudomonas aeruginosa is a Pseudomonas aeruginosa biofilm.

In various embodiments, the bacteriophage are selected from among the bacteriophage listed in Table 1.

In certain embodiments, the bacteriophage comprises a nucleic acid sequence selected from the group consisting of SEQ ID NOs: 1-13. In certain embodiments, the bacteriophage comprises a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleic acid sequence set forth in any one SEQ ID NOs: 1-13.

### Administration/Dosing

In the clinical settings, compositions of the invention can be administered to a subject by any of a number of methods, each of which is familiar in the art. For instance, a pharmaceutical formulation of the composition can be administered by inhalation, topically, locally or systemically, e.g., by intravenous injection, intramuscular injection, intraperitoneal injection, retro- or peribulbar injection.

The regimen of administration may affect what constitutes an effective amount. The therapeutic formulations may be administered to the subject either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the composition of the present invention to a subject, such as a mammal, for example a human, may be carried out using known procedures, at dosages and for periods of time effective to treat a disease or condition in the subject. An effective amount of the composition necessary to achieve a therapeutic effect may vary according to factors such as the extent of implantation; the time of administration; the duration of administration; other drugs, compounds or materials used in combination with the composition; the state of the disease or disorder; age, sex, weight, condition, general health and prior medical history of the subject being treated; and like factors well-known in the medical arts. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the composition without undue experimentation.

Actual dosage levels of the cells in the pharmaceutical formulations of this invention may be varied so as to obtain an amount of the composition that are effective to achieve the desired therapeutic response for a particular subject, composition, and mode of administration, without being toxic to the subject.

### Routes of Administration

Routes of administration of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, topical, intra-orbital, intra-aural, intra-articular, and topical administration.

Suitable formulation of the compositions and dosages include, for example, dispersions, suspensions, solutions, beads, pellets, magmas, creams, pastes, plasters, lotions, discs, suppositories, liquid sprays for nasal, ocular or oral administration, aerosolized formulations for inhalation, compositions and formulations for intravesical administration and the like.

It should be understood that the formulations and compositions that would be useful in the present invention are not limited to the particular formulations set forth in the examples. The following examples are put forth so as to provide those of ordinary skill in the art with a complete disclosure and description of how to make and use the cells, differentiation methods, engineered tissues, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

Bacterial growth conditions and media: Bacteria were grown in LB broth with 10 g tryptone, 5 g yeast extract, and 10 g/L NaCl. LB agar included 15 g/L agar and LB top agar included 7.5 g/L agar unless otherwise noted. Overnight culture incubation was performed at 200 RPM shaking at 37° C.

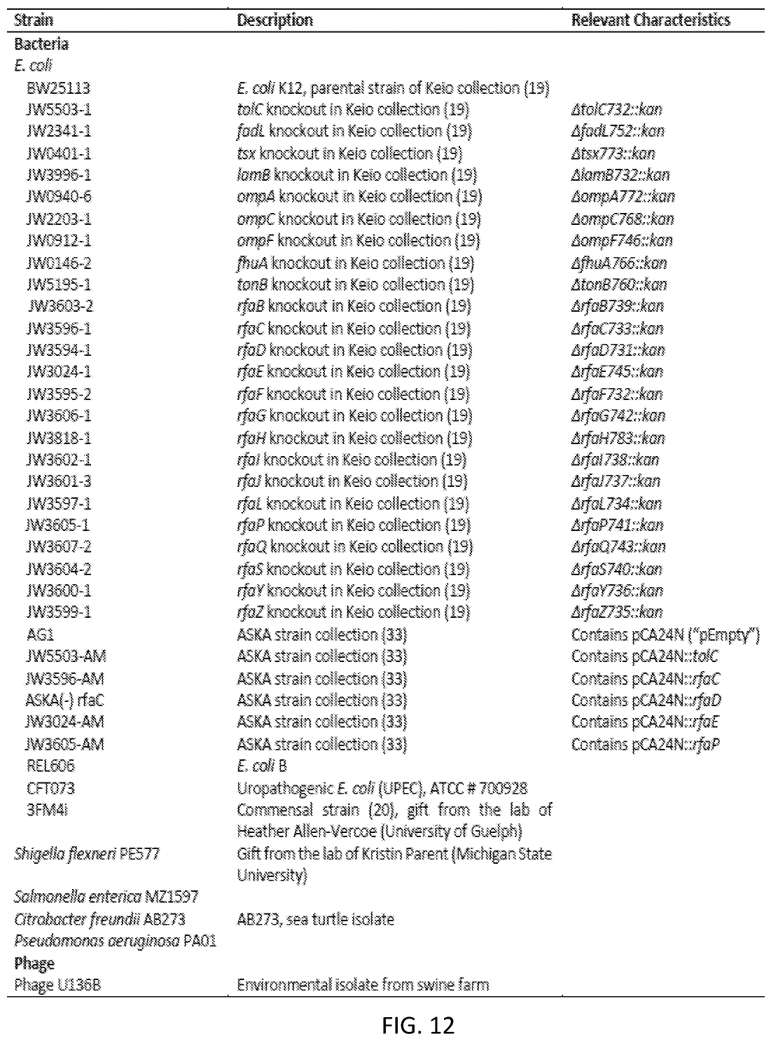

Phage library screens: To isolate phage U136B, thirty-three phage isolates collected previously from various sources were screened. Each isolate was screened using the plaque spot test on host lawns of wild-type and knockout E. coli from the Keio collection obtained from the Yale Coli Genetic Stock Center (FIG. 12). Those phages for which a difference in plaquing was observed in the library screen were then screened for differences in efficiency of plating (EOP, the number of plaques formed on a mutant divided by the number of plaques formed on BW25113). The initial EOP for phage U136B on the tolC knockout was determined using a full plate dilution series in top agar that contained 3.8 g/L agar and serial dilution spot tests of 2 μL to obtain phage titers on each bacterium.

Phage TIVP-H6 (Myovirus) was discovered by screening for phages that infect Pseudomonas aeruginosa strains which overproduce the virulence factor pyocyanin, and determining that evolution of phage resistance led to reduced pyocyanin production in bacterial mutants.

Phages LPS-5 (Podovirus) and LPS-TLTL were discovered by screening for phages that interact with LPS of P. aeruginosa, indicated by classic ‘rough-colony-forming’ phenotypes when phage-resistant mutants grow on agar (Luzar and Montie (1985) Infection and Immunity 50:572-576).

Phages SF37B, SF60B, SFNHSI, and SFA1-1 were discovered by screening phage isolates from various sources on Shigella strains PES77 and/or BW25113.

Phage PG-I1 was discovered by screening phage isolates from various sources on Staphylococcus aureus bacteria.

Mutant selection procedure: To select for phage-resistant bacteria, wild-type bacteria and phages were mixed, then plated onto LB agar and incubated overnight at 37° C. To ensure the isolation of independent mutations, each replicate was grown from a single isolated colony. To confirm the multiplicity of infection (MOI, the ratio of phage particles to bacterial cells) on the agar plate, bacteria and phage were plated in triplicate and counted cfu/mL and pfu/mL, respectively. From each mutant-selection plate, a random colony was picked, plus any colonies of notable morphology, and streaked onto LB agar plate and incubated at 37° C. overnight. The colonies were re-streaked to obtain double-purified isolates and each grown in 10 mL LB at 37° C. with shaking overnight. Freezer stocks of each mutant were archived in 20% glycerol, stored at −80° C.

Assessment of phage resistance: 10 μL of a high titer stock of phage (˜10⁹ pfu/mL) was streaked along ˜10 cm lines on LB agar plates. Phage streaks were allowed to dry, and 1 μL samples of overnight cultures of each bacterium were streaked perpendicularly across the phage lines in duplicate. Plates were allowed to dry and incubated overnight at 37° C. Bacterial streaks were scored for signs of phage lysis. Isolates with signs of lysis were scored as susceptible to phage while isolates without lysis were scored as phage resistant.

Bacterial growth curves for phage resistance assays: Cultures of wild type and other bacteria (e.g., phage-resistant and gene-knockout strains) were grown to exponential phase as measured by optical density at 600 nm wavelength (OD600) of 0.25-0.38. The exponential-phase cultures were diluted 1:5 into wells with LB to a total volume of 200 μL with phage to reach the target MOI. Cultures were incubated at 37° C. with shaking at 288 RPM for 18 h and OD600 read every 2 minutes using an automated spectrophotometer (TECAN microplate reader).

Bacterial growth curves for comparison of differing mutants: Bacterial cultures were preconditioned in LB medium overnight from individual colonies, then transferred to fresh LB in 200 μl total volumes. Cultures were incubated at 37° C. with shaking and optical density was monitored at 5-min intervals for 24 h by a TECAN microplate reader.

Single step growth curves: Overnight cultures of bacteria were diluted 1:100 into 10 mL pre-warmed LB broth and subjected to shaking at 200 RPM at 37° C. until exponential phase. These cultures were then inoculated into pre-warmed LB at an initial concentration of ˜2*10⁷ cfu/mL to begin the assay. Phage were added to an initial concentration of 2*10⁸ pfu/mL, for an initial MOI of 10. The assay flasks were incubated with shaking at 200 RPM at 37° C. Every 10 min, 50-μL samples were removed from each flask, transferred to a Spin-X column (CLS8160 Sigma), and spun at 14,000 rpm for 1 min. At 2 min and 12 min, bacterial samples were obtained from flasks with and without phage, and were diluted and plated in duplicate as above, in order to preliminarily assess infection rates. Within a week, each filtered phage sample was diluted in a 1:10 dilution series in sterile LB and 100 μL diluted samples were plated in soft agar overlay with 4 mL 7.5 g/L LB soft agar and 100 μL overnight bacteria (e.g., FIG. 12). Plates were incubated at 37° C. and plaques were counted the following day. For each time step, the dilution step with the greatest number of countable plaques was counted and used for analysis.

Transmission electron microscopy: Images of high-titer phage lysates were collected at the Yale Electron Microscopy facility in the Center for Cellular and Molecular Imaging. Samples were negatively stained with 2% uranyl acetate and imaged with FEG 200 kV transmission EM.

Broth microdilution method for antibiotic MICs: To examine colistin MIC, an original colistin solution (Alfa Aesar) with a concentration of 50 mg/mL was diluted to the following test concentrations in LB medium: 12.5, 25, 50, 100, 150, 200, 250, 300, 350, and 400 ng/mL. Bacterial cultures were grown from single colonies overnight in 10 mL of LB incubated at 37° C. with shaking. Each culture was diluted to ˜2*10⁵ cfu/mL in LB. MICs for colistin were measured by dispensing 100 μL of a given colistin concentration into each column and 100 μl of diluted bacteria into each row of a 96 well plate and incubating at 37° C. with shaking for 18 hr. Bacterial growth was scored visually as a binary variable (no growth vs. any visible growth). The lowest colistin concentration at which the well was completely clear was the value recorded as the MIC. For cases where replicates gave different answers, we report the mode.

Statistical analysis: All analyses were performed in R Studio (R Development Core Team (2010) R: A language and environment for statistical computing. (R Foundation for Statistical Computing, Vienna, Austria) using custom scripts.

Genome sequence analysis: Genome sequences were collected at the University of Pittsburgh Microbial Genome Sequencing (MiGS) center. Genomic DNA libraries were prepared using a modified Illumina Nextera library preparation (Baym M, et al. (2015) PLoS One 10(5):e0128036). Paired-end reads of 151 bp length were collected to a final coverage of ˜100-fold across the reference genome. Sequences were analyzed using breseq with default sequence end-trimming mode (Baym M, et al. (2015) PLoS One 10(5):e0128036; Deatherage D E & Barrick J E (2014) Methods Mol Biol 1151:165-188).

Complementation assays for gene knockouts: Electrocompetent bacterial cells were made by inoculating 10 mL LB broth with 100 μL of overnight bacterial cultures grown in LB broth. These cells were then incubated and shaken at 37° C. for 2-3 hours until they reached an OD600 of approximately 0.6. Next, the cells were centrifuged at 4,000 rpm for 10 min. The supernatant was poured off, then 10 mL of chilled 10% glycerol was added to the pellet. After resuspending the pellet by vortexing, the cells were centrifuged for 5 min at 4,000 rpm, then the supernatant was poured off. This glycerol wash cycle was performed 4 times. Finally, the cells were resuspended in 150 μL of 10% glycerol and were frozen at −80° C. or immediately electroporated. Plasmid DNA from ASKA clones (Kitagawa M, et al. (2005) DNA Res 12(5):291-299) (FIG. 12) was purified using the protocol found in the QIA Miniprep Handbook from Qiagen. The electrocompetent cells were then either transformed with an empty plasmid or complemented with a plasmid containing the gene which had been knocked out. These transformations were performed by thawing electrocompetent cells and plasmid DNA and adding 1-3 μL of DNA (<100 ng of DNA) to the electrocompetent cells. After being chilled, the mixture was pipetted into a chilled cuvette and electroporated. Immediately after electroporation, 500 μL of LB broth were added to recover the cells. The cells were then incubated for 1 hr at 37° C. with shaking. 100 μL of the electroporated cells were then plated on LB plates containing approximately 20 μL of 30 pg/mL chloramphenicol. These plates were then incubated overnight at 37° C. before isolation of transformed colonies.

Bacteriophage enumeration and efficiency of plating for complementation assays: Soft agar overlay tubes were prepared by mixing 4 mL 7.5 g/L soft agar, 4 μL of 30 p,g/mL chloramphenicol (for transformed strains only), 20 μL of 100 mM IPTG, and 100 μL of a bacterial overnight culture (grown in LB broth). These soft agar overlays were poured over LB plates with added chloramphenicol. For non-transformed strains, no chloramphenicol was added. A 1:10 dilution series was performed for 4 phage stocks from 10⁻¹ through 10⁻⁷, and 2 μL of each dilution from each phage stock was spotted onto every bacterial overlay. After these spots were completely dry, the plates were incubated overnight at 37° C. Finally, pfu/mL was calculated for each plate, and the EOP was calculated by dividing the pfu/mL of a test strain by that of the wild type.

Assessment of tetracycline resistance: Overnight cultures were diluted in 1:10 dilution series in sterile LB, and 2 μL spots of each dilution were immediately plated on LB agar plates containing varying concentrations of antibiotic: 0, 0.12, 0.25, 0.5, 1.0, 2.0, and 4.0 pg/mL tetracycline. Plates were allowed to dry and incubated for two days at 37° C. The MIC of tetracycline was recorded for each isolate as the concentration at which colonies no longer formed at the 10⁻⁵ spotted dilution. For cases where replicates gave different answers, the mode was reported.

Preparation of phage stocks for the evolution experiment: 20 μL of cryogenically frozen U136B phage stock was diluted in sterile LB broth in a 1:10 dilution series. 100 μL of each dilution was plated with 100 μL of an overnight culture of DH5a in 4 mL 7.5 g/L LB soft agar overlay on standard LB agar and incubated overnight at 37° C. Ten isolated plaques were randomly chosen from the 10⁻⁷ dilution plate, suspended in 750 μL sterile LB, and filtered through a 0.22 p,m Spin-X column (CLS8160 Sigma) at 14,000 rpm for 1 min. The ten phage stocks were enumerated by top agar overlay plating and subsequently diluted in sterile LB to a target concentration of 1.0*10⁷ pfu/mL. 200 μL samples of each replicate phage stock were combined in duplicate with 200 μL sterile 40% glycerol solution and frozen at −80° C.

Preparation of bacterial cultures for the evolution experiment: Cryogenically-frozen E. coli strain BW25113 was streaked onto LB agar and incubated overnight at 37° C. Twenty separate isolated colonies were randomly chosen and suspended in 10 mL sterile LB in separate flasks. Cultures were incubated overnight at 37° C., with 200 rpm shaking. Samples of these founding cultures were archived by combining 900 μL of each replicate bacterial culture with 900 μL sterile 40% glycerol solution in duplicate, and frozen at −80° C. The experimental populations were established by adding 9.8 mL sterile LB to 50 mL glass Erlenmeyer flasks and then inoculating with 100 μL of one of the BW25113 bacterial cultures described above (each flask receiving a distinct starting culture) to an estimated concentration of 2*10⁷ cfu/mL (assuming final E. coli overnight densities in LB are 2*10⁹ cfu/mL). For the +phage populations, 100 μL of one of the diluted U136B phage stocks prepared above was then added to 10 of the flasks (each flask receiving a distinct starting culture) to a concentration of 1*10⁵ pfu/mL. For the 10 control populations, 100 μL sterile LB broth were added in place of phage. Flasks were incubated overnight at 37° C., with 200 rpm shaking.

Serial passaging in the evolution experiment: Each day (24 h±2 h), the appearance of each flask was recorded and 100 μL from each experimental flask were transferred into 9.9 mL sterile LB broth in fresh flasks. To reduce risk of contamination, the transfer micropipette was wiped down with 70% ethanol on a between each transfer, and flasks with phage and without phage were alternated to enable detection of cross contamination. To archive the evolved samples each day, 900 μL samples of each replicate experimental volume were combined in duplicate with 900 μL sterile 40% glycerol solution and frozen at −80° C.

Daily enumeration of bacteria during evolution experiment: Each day, 1:10 serial dilutions of each experimental volume were prepared in sterile 0.85% NaCl up to 10⁻⁸, and 2 μL spots of each dilution were plated on both standard LB agar plates and LB agar plates prepared with tetracycline. Plates were allowed to dry and then incubated overnight at 37° C. The following day, the most dilute spot with colonies present was identified and colonies were counted in order to determine the total bacterial population density and the Tet^{R} bacterial population density for each replicate flask.

Daily enumeration of phage during evolution experiment: The dilution series prepared above was also used to enumerate phage daily. For the 10 flasks with phage, 2 μL spots of each dilution were plated with 100 μL of an overnight culture of DH5a in 4 mL 7.5 g/L LB soft agar overlay on standard LB round plates (when square plates were used, 125 μL DH5a was used in 5 mL soft agar) and incubated overnight at 37° C. For the flasks without phage, only the least dilute concentration was plated as a check for phage contamination. The following day, the most dilute spot with plaques present was identified and plaques were counted in order to determine the phage titer.

Isolation of bacteria from evolution experiment: Two individual isolates were obtained from each population at day 5 and day 10. Isolated colonies were selected in the most dilute spots that contained colonies on the bacterial enumeration plates described above. One colony was picked from the total bacterial population (on the standard LB plates) while one colony was picked from the LB plates containing 0.5 p,g/mL tetracycline. The colonies were streaked onto sterile LB plates and incubated overnight at 37° C. For each isolate, the colony most distant from its neighbors was streaked onto a new sterile LB plate and incubated overnight at 37° C. For each re-streaked isolate, the colony most distant from its neighbors was suspended in sterile LB broth and incubated overnight at 37° C., with 200 rpm shaking. To archive the isolated bacteria, 900 μL samples of each bacterial culture were combined in duplicate with 900 μL sterile 40% glycerol solution and frozen at −80° C.

Kirby Bauer colistin disk diffusion assay: Mueller Hinton plates were prepared by pipetting 25 mL of Mueller Hinton agar into each plate, then allowed to dry for two days. Plates were then used either immediately or refrigerated until use. Overnight bacterial cultures of each strain were prepared by incubating a single bacterial colony in 10 mL LB broth at 37° C. for 18-20 hours. After incubation, 1:10 dilutions of each bacterial strain were prepared by combining 900 μL 0.85% NaCl solution with 100 μL bacterial inoculation. To prepare the disk diffusion plates, 100 μL of each bacterial dilution were pipetted onto a Mueller Hinton plate then spread evenly across the plate with a sterile plastic L spreader. Plates were then allowed to dry and cardboard colistin disks (ThermoFisher, #CT0017B) containing 10 μg colistin were placed in the center of the plates using sterile forceps. Plates were incubated for 20-24 hours at 37° C. Finally, to measure the diameters of the zones of inhibition (ZOI), three straight lines, each at a different angle, were drawn through the center of the ZOI using a ruler. The diameter of the ZOI was measured along the drawn lines.

INSeq transposon mutant library preparation. The insertion-sequence (1NSeq) transposon mutant library was made via conjugation. A donor bacterial strain containing a plasmid was conjugated with a wild type recipient strain on agar plates, at a ratio of 1:1 donor to recipient. After 3 hours incubation at 37° C., conjugation mixtures were plated for single colonies on plates with antibiotic to isolate transconjugants that were resistant to both markers (i.e., marker on plasmid and on recipient chromosome). Following 24 hours incubation at 37° C., colonies were scraped into LB medium using a sterile spatula and stored in 20% glycerol at −80° C.

INSeq screen. Aliquots of the library were thawed on ice, washed once in LB and resuspended to a concentration of approximately 10⁷ CFU/mL in LB and incubated at 37° C. for 1 hour, with shaking. A sample of test phage was added, in triplicate, to a final concentration of approximately 10⁵ PFU/mL, for MOI≈0.01. Following overnight incubation, cultures were harvested and genomic DNA was extracted and prepared for sequencing. DNA libraries were pooled and sequenced at the Yale Center for Genome Analysis via the Illumina HiSeq2500 system. Sequences were analyzed using scripts modified from Goodman et al (Cell Host Microbe 6, 279-289 (2009)). Briefly, using Python scripts adapted from analysis packages previously described (Goodman et al. (2011) Nat Protoc 6, 1969-1980), sequencing reads were indexed by barcode, and transposon sequence was trimmed leaving 16 bp of adjacent genomic DNA. These 16 bp sequences were aligned to a reference genome using Bowtie2, by counting the number of reads for each insertion site, normalizing to counts per million reads and binning by gene (Langmead and Salzberg (2012) Nat Methods 9, 357-359). Transposon insertions mapping to the distal 5% ends of any coding region as well as transposon insertions mapping to intergenic regions were filtered out during analysis. A Z-test was performed using the log ratio of normalized output count to normalized input count, and Q-values from false discovery rate (FDR) correction of <0.05 were considered significant for further analysis.

Efficiency of plating, and adsorption assays. Efficiency of plating (EOP) was measured as the ratio of the test phage titer on the experimental strain to its titer on the WT strain. Phage binding to cells (adsorption) was measured over a 20 minute adsorption assay (Kropinski. Bacteriophages. (Humana Press, 2009), pp. 151-155), which estimates the rate of ‘disappearance’ of phage particles (i.e., reduction in titer) in liquid LB medium over time when viruses are challenged to attach to bacterial cells.

The results of the experiments are now described.

### Example 1

Bacteria frequently encounter selection by both antibiotics and lytic bacteriophages. However, the evolutionary interactions between antibiotics and phages remain unclear, in particular whether and when phages can drive evolutionary trade-offs with antibiotic resistance. Herein, Escherichia coli phage U136B is described. Its reliance on two host factors involved in different antibiotic resistance mechanisms: 1) the efflux pump protein TolC, and 2) the structural barrier molecule lipopolysaccharide (LPS), was demonstrated. Since TolC and LPS contribute to antibiotic resistance, phage U136B should select for their loss or modification, thereby driving a trade-off between phage resistance and either of the antibiotic resistance mechanisms. To test this hypothesis, fluctuation experiments and experimental evolution were used to obtain phage resistant mutants. Using these mutants, the accessibility of specific mutations (revealed in the fluctuation experiments) were compared to their actual success during ecological competition and coevolution (revealed in the evolution experiments). Both tolC and LPS-related mutants arose readily during fluctuation assays, with tolC mutations becoming more common during the evolution experiments. In support of the trade-off hypothesis, phage resistance via tolC mutations occurred with a corresponding reduction in antibiotic resistance in many cases. However, contrary to the hypothesis, some phage resistance mutations pleiotropically conferred increased antibiotic resistance. The molecular mechanisms underlying this surprising pleiotropic result, consideration for practical aspects of phage application, and the importance of ecology in evolution of phage resistance are discussed herein. It is envisioned that phages are useful for the reversal of antibiotic resistance, but that such applications will need to account for both unexpected pleiotropy and evolutionary context.

### Example 2: Phage U136B Relies on the Antibiotic Resistance Protein TolC

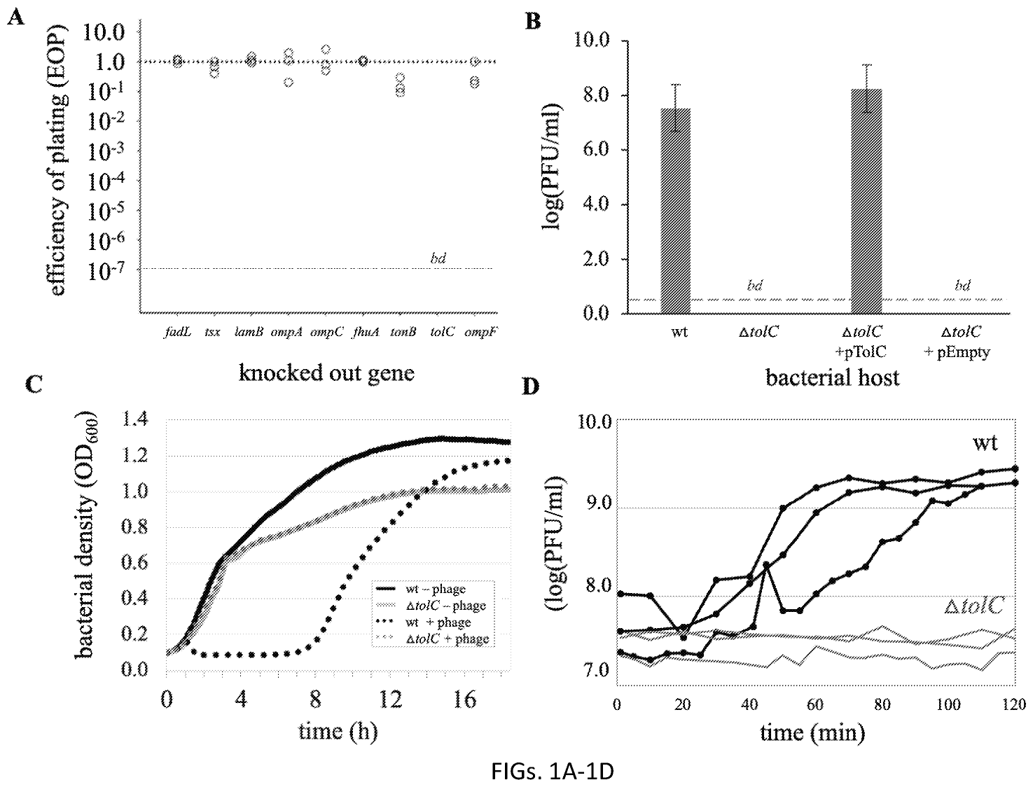

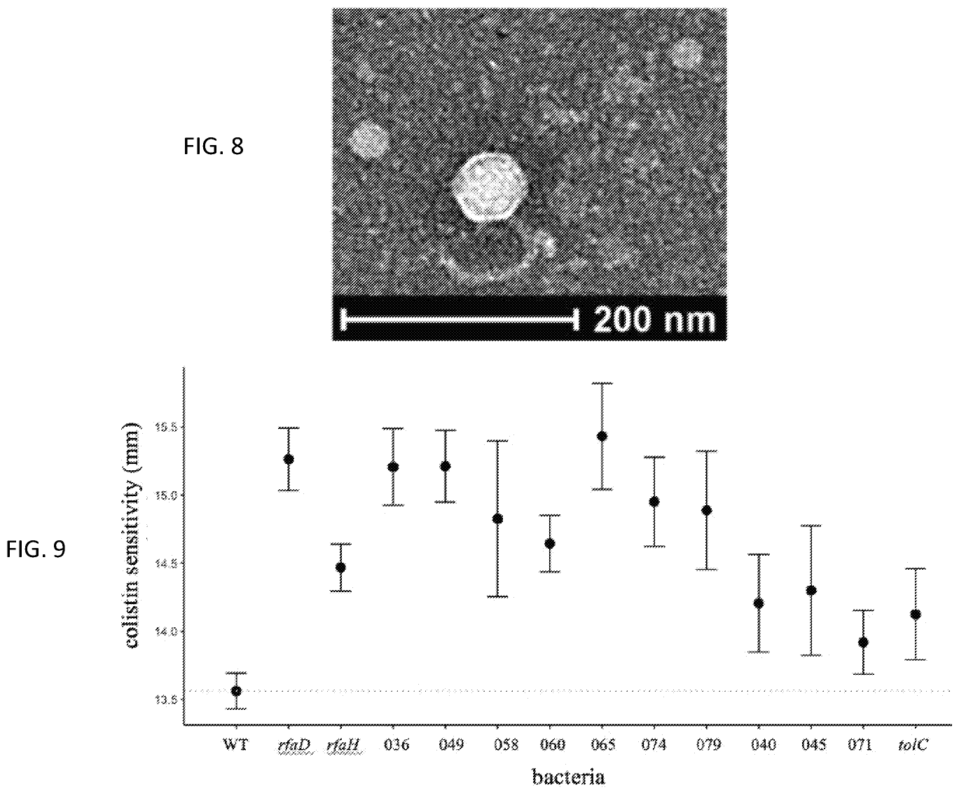

To identify phages that use the antibiotic efflux pump protein TolC for infection, a collection of E. coli phages were screened for the inability to form plaques on a tolC knockout. This screen identified phage U136B, a curly-tailed phage with siphophage morphology (FIG. 8) that had been originally isolated from a pig farm in Connecticut (FIG. 12) and has no plaquing ability on a tolC knockout (FIG. 1A). Knocking out outer membrane proteins used by other phages had no impact on phage U136B infection, indicating tolC is unique in this respect. Expression of tolC from a plasmid vector fully restored phage U136B plaquing on the tolC knockout (FIG. 1). Phage U136B also had no effect on the growth of tolC knockout bacteria in liquid culture (FIG. 1C), and correspondingly the phage did grow on the tolC knockout (FIG. 1D).

### Example 3: Phage U136B Relies on Inner Core of the Lipopolysaccharide

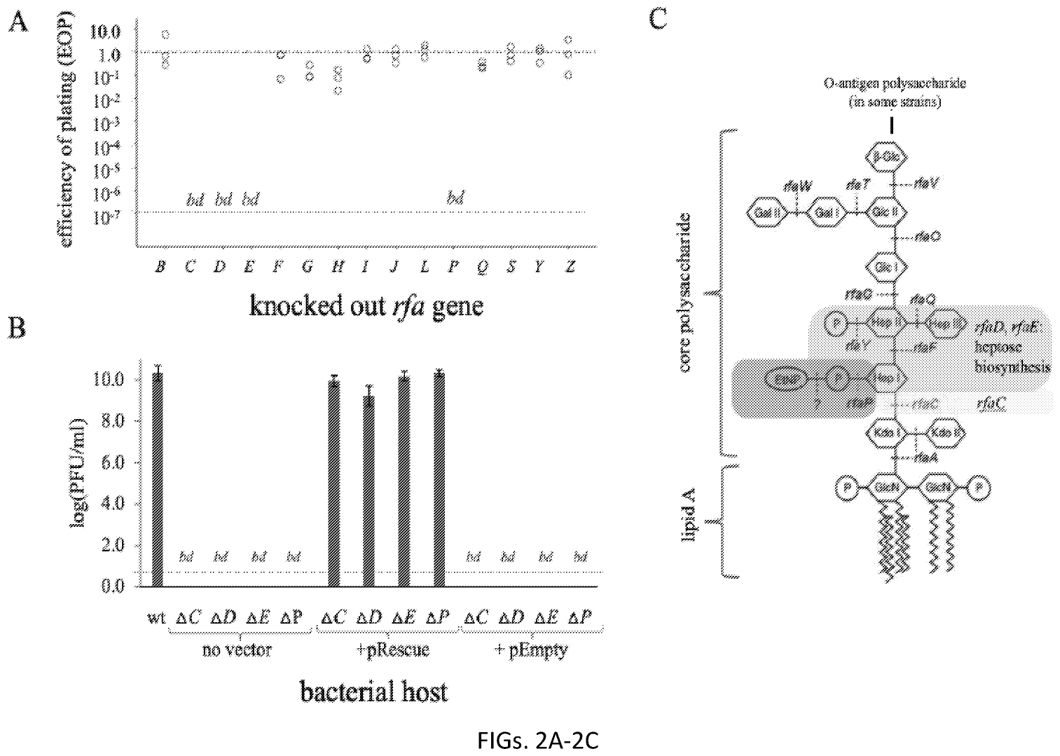

In addition to outer membrane protein receptors, many phages also require LPS receptors in their initial contact with the host cell. To assess whether U136B relies on LPS, a screen was conducted on a panel of genes involved in LPS synthesis (the “rfa” genes, named originally for the rough (′rf) colony phenotype of some strains with altered LPS structures). All of the rfa gene knockouts available in the Keio collection were used (Baba T, et al. (2006)Mol Syst Biol 2:2006 0008) (FIG. 12). Four of the fourteen rfa genes were required by U136B (FIG. 2A). It was confirmed that these genes were required for plaque formation using genetic complementation tests (FIG. 2B). These four genes (rfaC, rfaD, rfaE, and rfaP), were all involved in formation of the core region of the LPS polysaccharide component (FIG. 2C). Together, these results show that U136B likely requires the inner core of the lipopolysaccharide. Because knocking out either tolC alone or any of the rfa genes alone provides full protection against U136B, both TolC and LPS appear to be necessary for infection, rather than an “either/or” mechanism.

### Example 4: Phage Host Range is Limited to a Subset of E. coli Strains

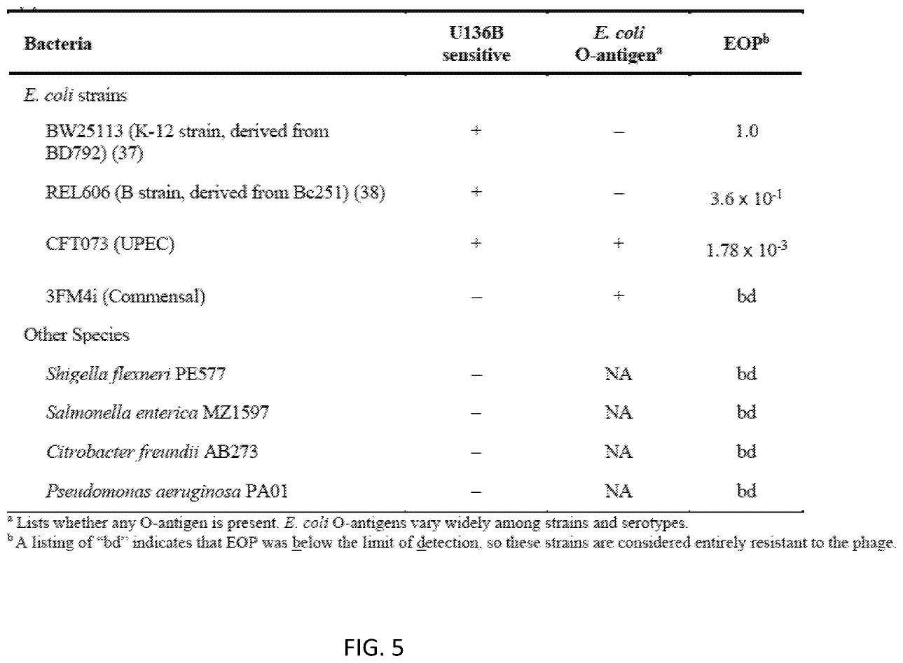

Phage host range depends largely on receptor availability and receptor structure, so host specificity can be constrained within species. U136B's host range was tested on a panel of divergent E. coli strains and more distant Gram-negative bacteria relatives (FIG. 12). Among the strains tested for plaquing ability, U136B's host range was limited to E. coli and had strain specificity, with plaquing ability on E. coli K, E. coli B and UPEC, but not on a commensal isolate (FIG. 5).

### Example 5: Phage U136B Selects for Altered Cell Surface Factor Genes

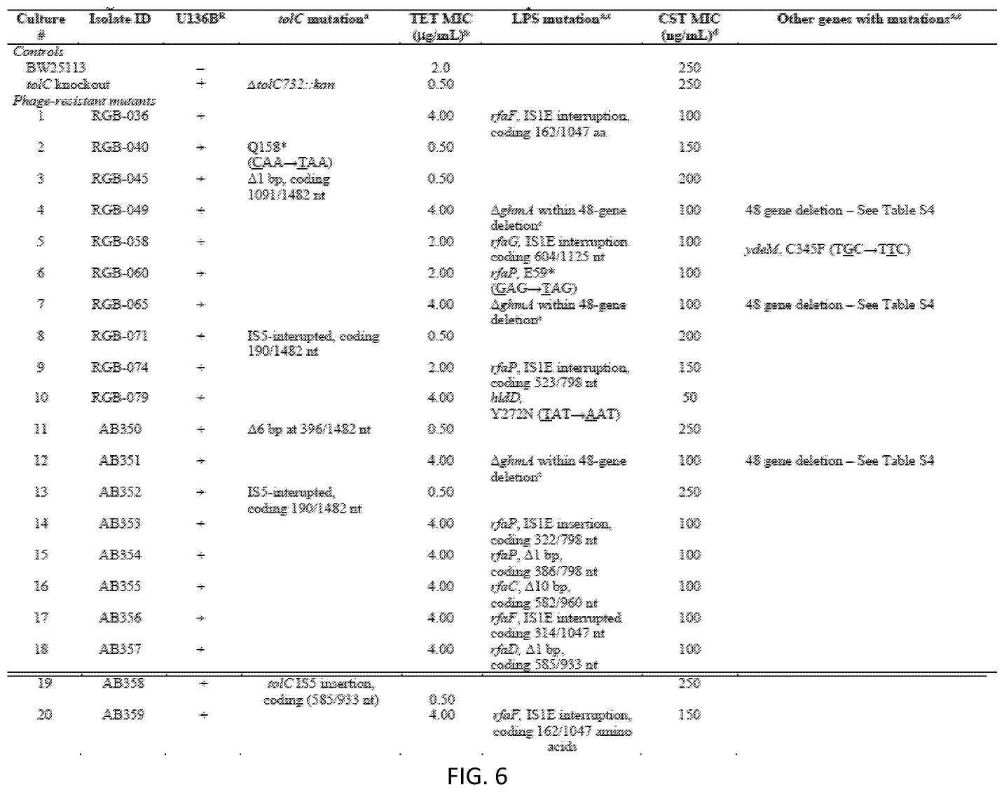

E. coli phage resistance mutations were examined to determine potential evolutionary paths bacterial populations may take when evolving in the presence of phage. To do this, modified fluctuation assays were used, which reveal pre-existing phage resistance mutations that have had minimal time to compete with one another. Twenty independent cultures of E. coli BW25113 (K strain, U136B^{S}, Tetracycline^{R}, Colistin^{R}) were plated with phage U136B, yielding a mutant frequency of 4.0×10⁻⁶. One phage-resistant mutant colony was randomly picked from each culture. After streaking for double-isolation, the genomes of each mutant were sequenced. Either tolC mutations or LPS-related mutations were observed in 100% of these mutants (FIG. 6), with LPS-related mutations occurring more frequently (70%, ˜2.8×10⁻⁶) than tolC mutations (30%, ˜1.2×10⁻⁶).

### Example 6: Phage Resistant Mutants have Altered Sensitivity to Antibiotics

To determine if the phage resistant mutants had altered resistance to antibiotics, in particular decreased antibiotic resistance via antagonistic pleiotropy, their minimum inhibitory concentrations (MICs) for tetracycline and colistin were determined and compared to their wild-type, phage-sensitive parental strains. Tetracycline is an antibiotic that binds to the bacterial ribosome and prevents peptide chain elongation. Resistance to tetracycline can occur by efflux through the TolC-AcrAB efflux pump, and it was predicted that phage resistance mutations in tolC would result in decreased resistance to effluxed antibiotics. Colistin is a polypeptide antibiotic in the polymyxin class and used as a drug of last resort. Previously, a large screen of E. coli knockouts suggested that changes to several of the rfa LPS synthesis genes may result in increased colistin resistance (summarized in FIG. 6); here, it was predicted that phage resistance mutations in rfa genes would result in decreased resistance to colistin.

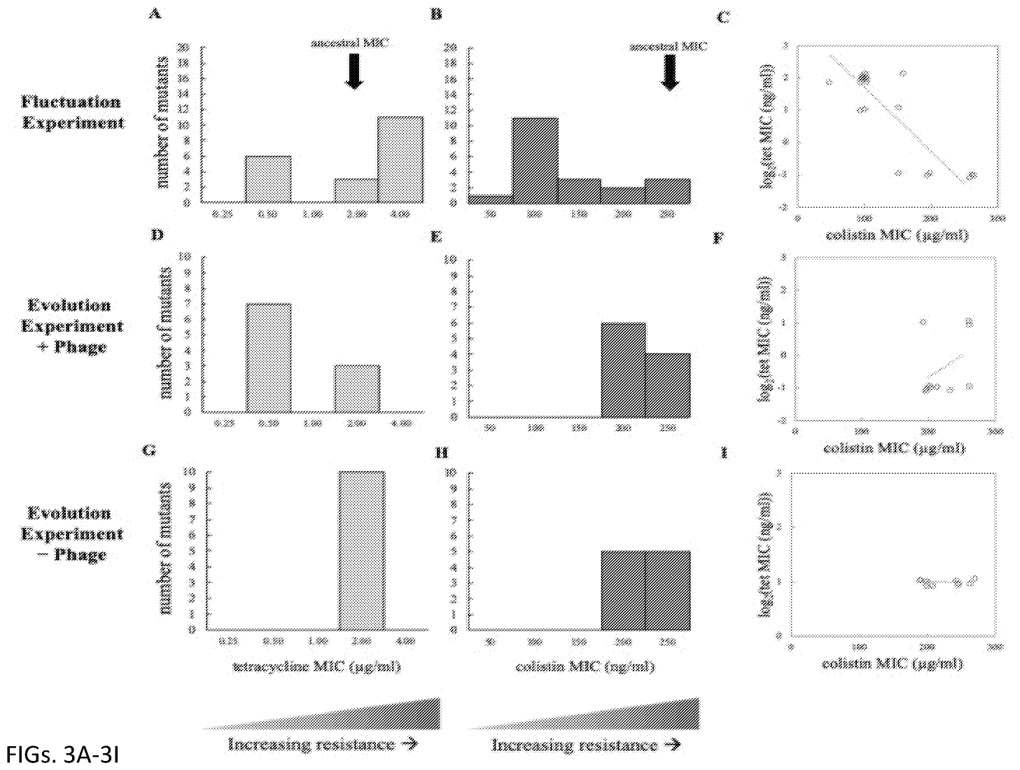

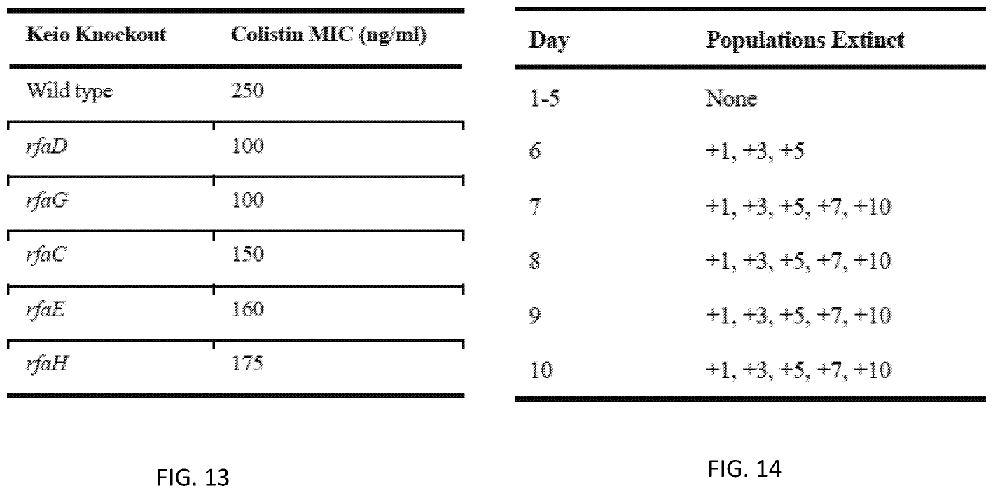

Supporting the antagonistic pleiotropy hypothesis, all six tolC mutants had reduced resistance to tetracycline compared to the parental strain and a similar phenotype to the tolC knockout control (FIG. 3A, FIG. 6). Some of the tolC mutants also had reduced colistin resistance (FIG. 3B, FIG. 6, FIG. 13). Meanwhile, the other phage-resistant mutants with LPS-related mutations had decreased resistance to colistin (FIG. 3B, FIG. 6, FIG. S13) compared to the parental strain. Surprisingly, a subset of the LPS mutants had increased resistance to tetracycline (FIG. 3A, FIG. 6) at the same time as their reduced resistance to colistin. This result indicates that the mutant LPS also has a pleiotropic effect on tetracycline resistance (FIG. 3C, Table 17).

### Example 7: Antibiotic Sensitivity Evolves in Populations with Phage Selection

Fluctuation experiments revealed bacterial mutations in tolC and LPS-synthesis genes, so it was tested if these same types of mutations would be selected in evolving communities of bacteria and phage. Compared to fluctuation experiments where phage-resistant bacteria have not competed with one another, evolution experiments expose the fitness costs of mutations to selection, altering mutation frequencies over time. The phage resistance mutations may vary in their general fitness costs, in their degree of phage resistance, or the potential for phages to counteract the various resistance mechanisms via coevolution. It was expected that the tolC mutations may arise more quickly and outcompete the LPS-synthesis mutants, as growth curve data demonstrated that tolC mutants are generally more fit than LPS-synthesis mutants (FIG. 14).

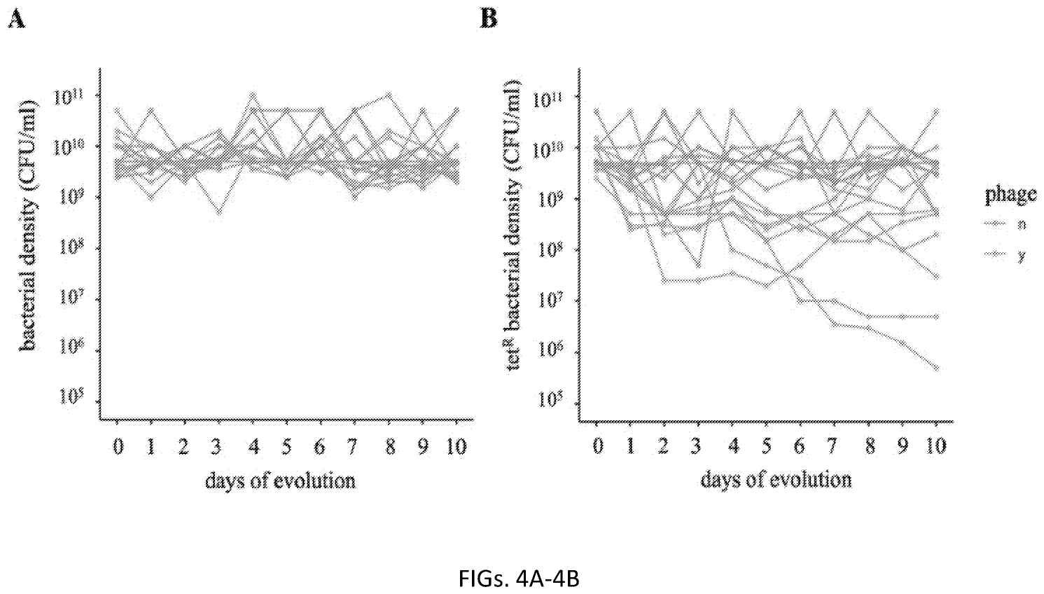

To test these ideas, a 10-day serial-passaging evolution experiment was conducted. Twenty populations with E. coli BW25113 were founded: 10 populations with phage U136B (“+phage”) and 10 control populations without phage (“−phage”), each passaged serially for 10 days. Each day, total bacterial density (FIG. 4A) and tetracycline-resistant bacterial density (FIG. 4B) was enumerated in real time by plating population samples upon serial transfer. During serial passaging, phage densities were monitored on a daily basis. Phage persisted in all populations through Day 5, with half of the phage populations going extinct by Day 10 (FIG. 14).

Again consistent with antagonistic pleiotropy, the populations evolved with phage U136B show a decreased frequency in the tetracycline-resistant phenotype relative to total population size. This effect was present both before phage extinctions occurred on Day 5 (FIG. 4B, t(10.1)=−6.0, p<0.0001, t-test of unequal variance) and at the end of the experiment on Day 10 (FIG. 4B, t(11.4)=−3.0, p<0.01, t-test of unequal variance). The total bacterial population size was unchanged by phage treatment both before phage extinctions (FIG. 1A, t(18.0)=0.82, p=0.789, t-test) on Day 5 and at the end of the experiment (FIG. 1B, t(11.7)=−1.27, p=0.11, t-test of unequal variance) on Day 10.

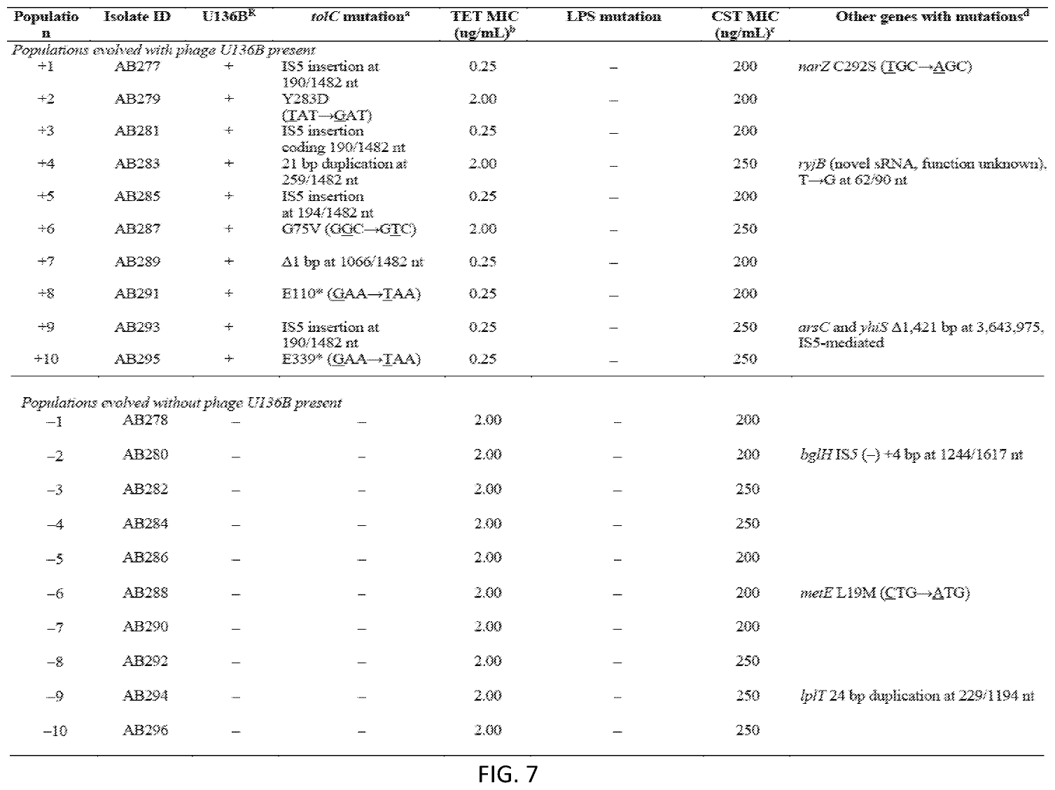

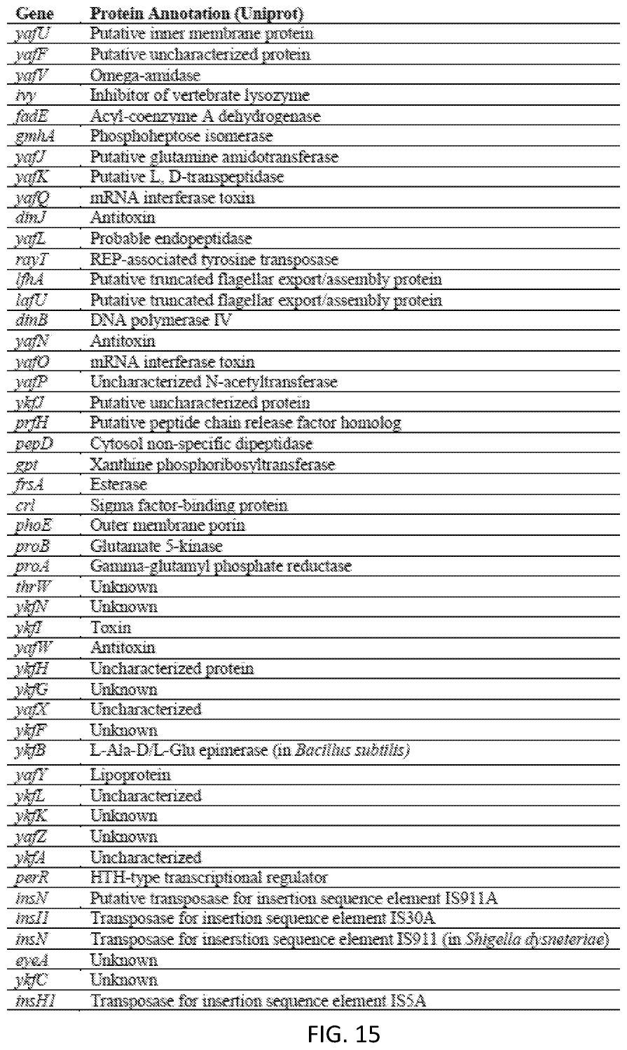

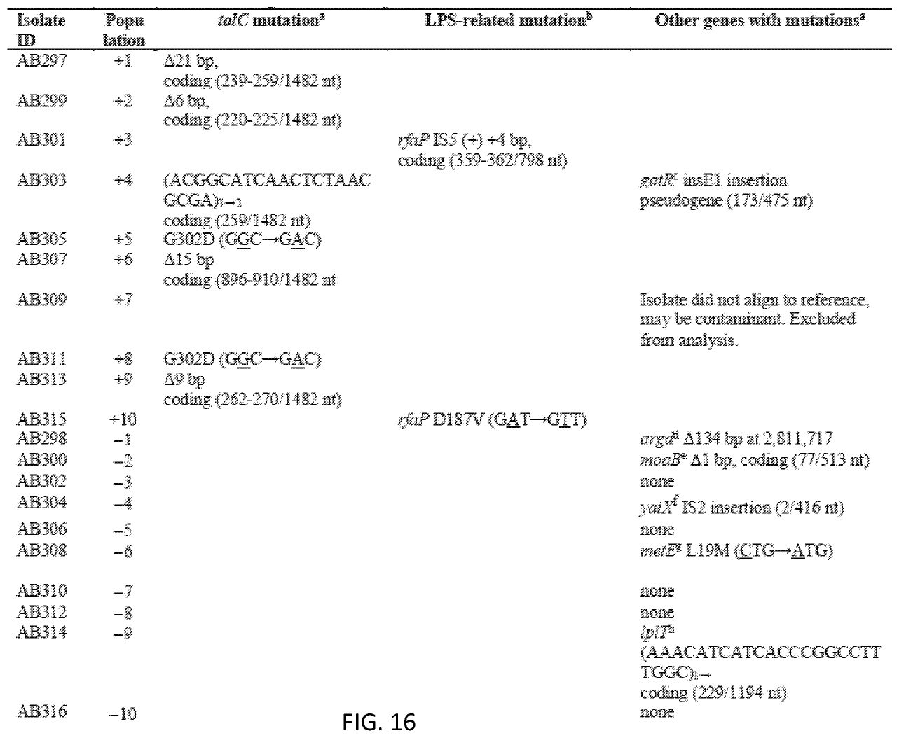

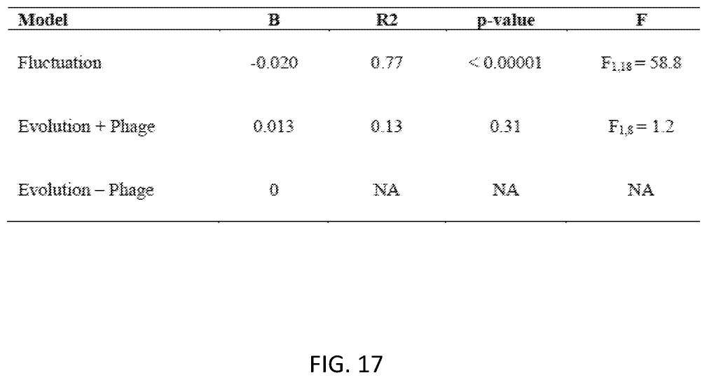

To investigate the genetic basis of adaptation and patterns of pleiotropy in the evolved populations, one random bacterial clone from each of the control (−phage) and treatment (+phage) populations was isolated on Day 10. Consistent with the hypothesis that +phage populations would face selection against TolC, after 10 days of evolution, the isolates from the +phage populations showed a predominance of tolC mutations and no LPS-related mutations (FIG. 7). However, the unexpected result that 3/10 of these evolved tolC mutants also retained the ancestral tetracycline (Tet) resistance phenotype was also encountered (FIG. 3D, FIG. 7: Tet^{R}=R), indicating these phage resistance mutations had no pleiotropy effect for tetracycline resistance. Those mutations mapped in or near the surface-exposed loops of TolC (FIG. 15), similar to mutations isolated under simultaneous selection by phage TLS and novobiocin. Few mutations in the −phage control populations were observed, with no mutations in either LPS or tolC. Only isolates evolved in the presence of phage U136B were resistant to the phage (FIG. 7, U136B^{R}), showing that the phage selection was responsible for the evolution of these alleles, and not a pleiotropic effect of selection in this general environment. The isolates from the control populations also had largely unchanged MICs for tetracycline and colistin (FIG. 3G, 3H, 3I). To follow-up on the tetracycline-resistant population members, isolates were obtained from cultures plated on tetracycline-containing agar, revealing that the tolC mutations do not reach fixation in all +phage populations (FIG. 16). Most strikingly, after 10 days of evolution none of the random +phage population isolates had increased tetracycline resistance, as was observed for some of the fluctuation isolates (FIG. 3A vs 3D, FIG. 17).

### Example 8: Discussion

The interactions between bacteriophages, antibiotic-resistant bacteria, and antibiotic-sensitive bacteria are complex. One reason detailed knowledge about their ecological and evolutionary interactions is lacking is that few phages have been tested for interactions with antibiotic resistance genes. To gain insight into such relationships, a collection of 33 environmental and commercial E. coli phages were screened for their reliance on the antibiotic efflux pump gene tolC. The screen identified phage U136B (FIG. 5), which relies on both the antibiotic resistance gene tolC (FIG. 1) and the core of the LPS (FIG. 2). Fluctuation assays were used to find that phage U136B selects for host mutations in genes encoding its critical host entry factors, tolC and LPS (FIG. 6). These phage-resistant mutations had pleiotropic changes to their sensitivity to tetracycline (mediated by tolC changes), colistin (mediated by LPS-related changes), or both (FIG. 6, FIGS. 3A-3B), including both predicted cases of antagonistic pleiotropy as well as surprising cases of synergistic pleiotropy. Phage resistance mutants and their associated antibiotic sensitivity phenotypes also evolved in communities of E. coli and phage U136B (FIG. 7, FIGS. 3D-3E).

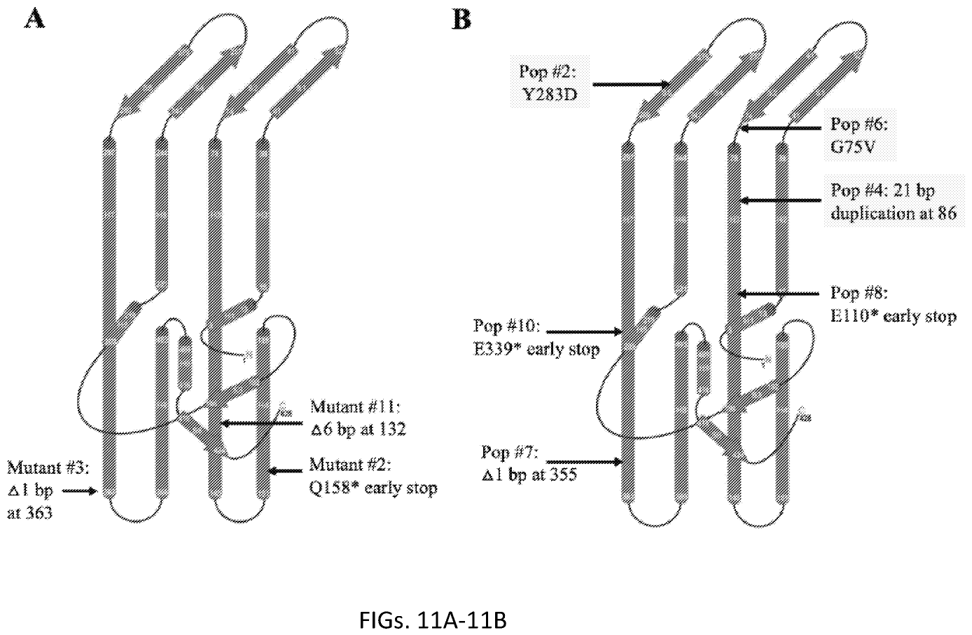

The role of competition and community in pleiotropic evolution: Comparing the mutations and phenotypes that arise during fluctuation experiments to those from evolution experiments provides an opportunity to compare the accessibility of specific mutations (in this case, tolC and LPS-related mutations) to their actual success during ecological competition and coevolution. Fluctuation experiments revealed that both tetracycline-sensitive (tolC) and colistin-sensitive (LPS-related) mutants arise readily, with LPS-related mutants dominating numerically (FIGS. 3A, 3B, & 6), likely because this complex, multi-locus trait has more mutational targets than the single tolC gene. In contrast, after 10 days of evolution in a community with phage U136B, LPS-related mutants were relatively rare, appearing in none of the random isolates from the 10+phage populations. This effect was mediated by fitness differences between tolC and LPS-related mutations (FIG. 11). Overall, these results revealed that competition can result in different outcomes than might be expected from a survey of accessible mutations.

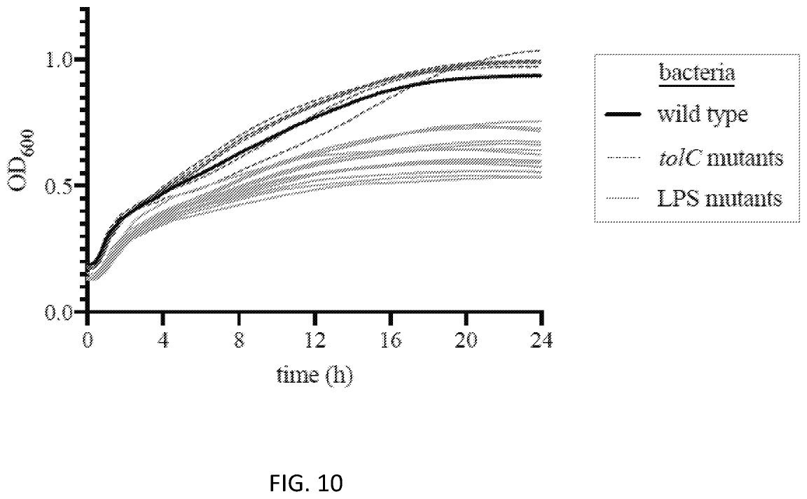

The outcomes of competition over evolutionary time will also be influenced by how long phage persist in the treatment environment. While all 10 of the +phage populations started under identical conditions and contained tolC mutants, these populations otherwise widely varied in terms of phage persistence (FIG. 14), the frequency of tetracycline resistance (FIG. 4B), and the specific mutations observed (FIG. 7). Strikingly, the final densities of tetracycline resistance varied by nearly four orders of magnitude across the populations. Together, these results suggest that development of predictable therapeutic phage population dynamics may be complicated, especially since laboratory conditions will be much less variable than those within and among patients, which would further amplify differences in phage persistence. Furthermore, the dynamics of tetracycline resistance in the +phage populations also varied both before and after phage extinctions, suggesting that the among-population differences were driven in part by the dynamics of competition among the host mutations within the populations (FIG. 10).

Obtaining a better understanding of community evolution and ecological variability is important in predicting dynamics of therapeutic phages in patients, especially since these complex environments contain other community members that can impact evolutionary outcomes. Such interdisciplinary work may include incorporating evolutionary factors and environmental heterogeneity into computational models of phage infection, tracking phage population sizes in infection models and clinical trials, and including both pharmacokinetic/pharmacodynamic (PK/PD) and phage dosing (rather than relying on a single initial inoculum). Together, these approaches may help to better control phage infection and limit persistence to therapeutic windows.

The effects of specific phage-resistance mutations on pleiotropic traits: The specific tolC mutations observed varied in their effects on the gene as well as on the resulting tetracycline phenotype. Two types of tolC phage-resistance mutations were observed: one type that lost the efflux function of TolC, and another type that retained the efflux function (FIG. 7, FIG. 16). All of the evolved tolC alleles that retained efflux function contained either nonsynonymous substitutions or small in-frame indels. These mutants were observed only after a period of evolution, suggesting that TolC function may be under weak selection even in the absence of antibiotic, for example through TolC's role in dealing with oxidative stress. However even tolC alleles that lost efflux function retained rapid growth rates and high cell densities (FIG. 10), so the importance of non-efflux TolC functions appears to be weak.

Data presented herein reveal that phage resistant mutants with LPS-synthesis gene mutations have decreased resistance to the antibiotic colistin. These data are consistent with the results of a high-throughput screen that identified some rfa genes involved in colistin resistance (FIG. 9). Colistin's mechanism of action is disruption of the bacterial outer membrane via interactions with LPS. It is possible that phage-selected mutations in rfa genes change the LPS structure in such a way that makes it more susceptible to colistin disruption, resulting in the evolutionary trade-off between phage resistance and colistin resistance. Although LPS-related mutations were generally less abundant than tolC mutations after experimental evolution with phage U136B, the LPS mutants did not necessarily go extinct (FIG. 16), indicating their potential importance in phage treatments of infections.

LPS-related changes may also have unintended, negative consequences in pathogenic bacterial populations. The mutation screen uncovered phage-resistant mutants that had increased resistance to tetracycline through changes to LPS (FIG. 6), likely due to a combination of decreased permeability of the outer membrane and reduced expression of OmpF, tetracycline's uptake porin. However, not all LPS-related mutants from the initial screen had increased tetracycline resistance relative to the wild type, suggesting the potential to nudge LPS evolution in a direction amenable to reducing antibiotic resistance. The fitness cost of LPS-related mutations is also more severe than that for the tolC mutations (FIG. 10), indicating that a general trade-off between LPS-related phage resistance and bacterial growth may further constrain their evolution.

The evolution of pleiotropic traits beyond antibiotic resistance: LPS is a crucial determinant of host range and medically relevant phenotypes like antigenicity and virulence. Many phages currently being developed for therapeutic use are reliant on LPS, so understanding the evolutionary implications of phage-LPS interactions is important in predicting if and when LPS phage selection may change bacterial virulence and resistance phenotypes. In particular, these interactions are important to predicting how phages will interact with their hosts, including potential collateral damage to commensal bacteria and the evolution of host-range expansion during treatment. Additionally, if LPS-related phage resistance also results in reduced LPS-associated virulence, then this resistance mechanism might be a useful means for reducing bacterial load while also evolutionarily reducing infection severity.

Overall, these results highlight the importance of considering community-level effects, such as competition, when measuring pleiotropic effects of phage resistance. Phage U136B and others like it are useful for the direct treatment of pathogenic infections.

### Example 9

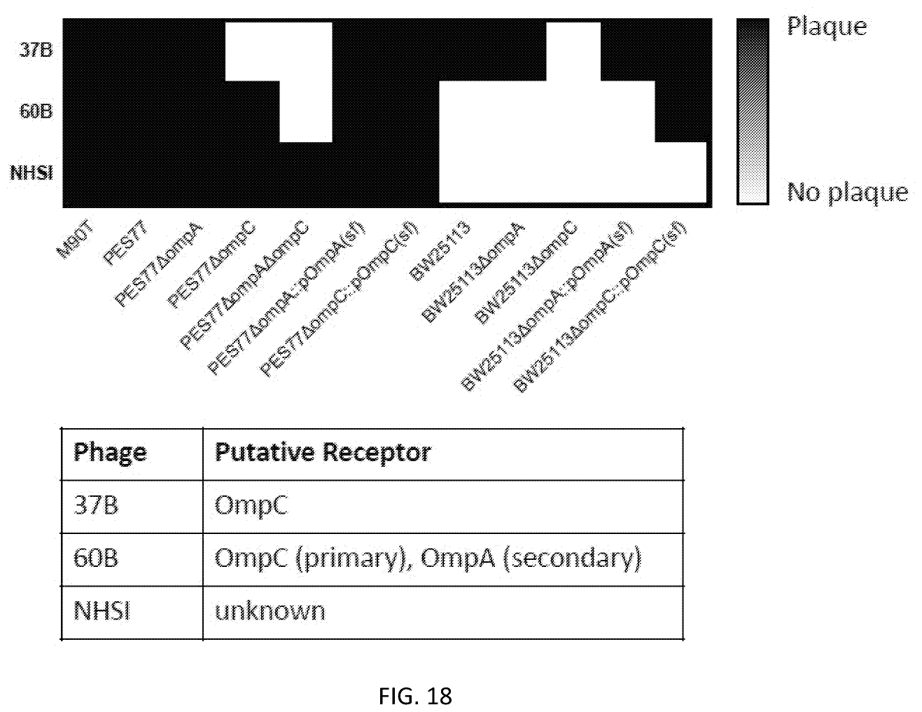

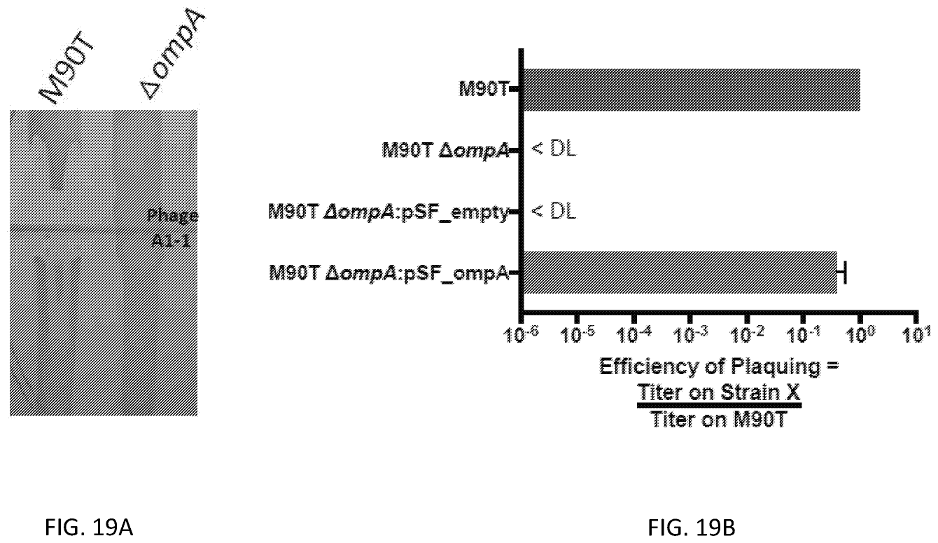

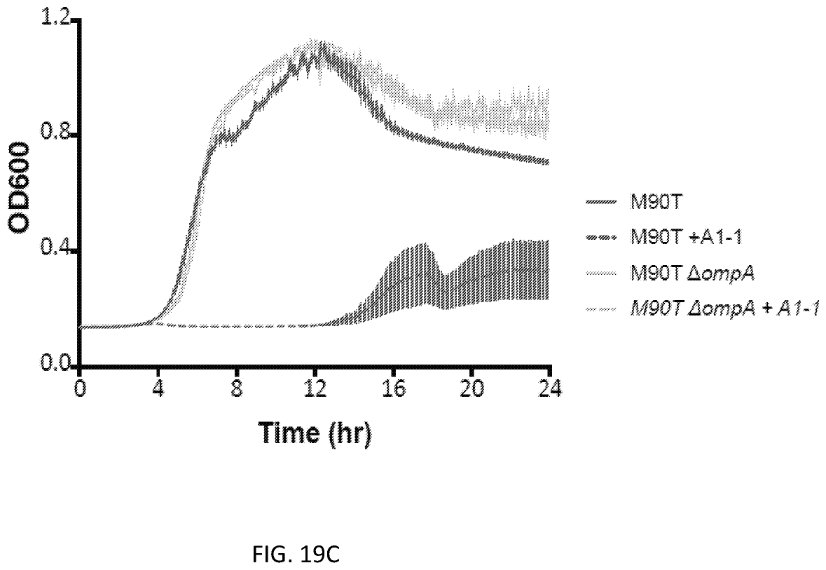

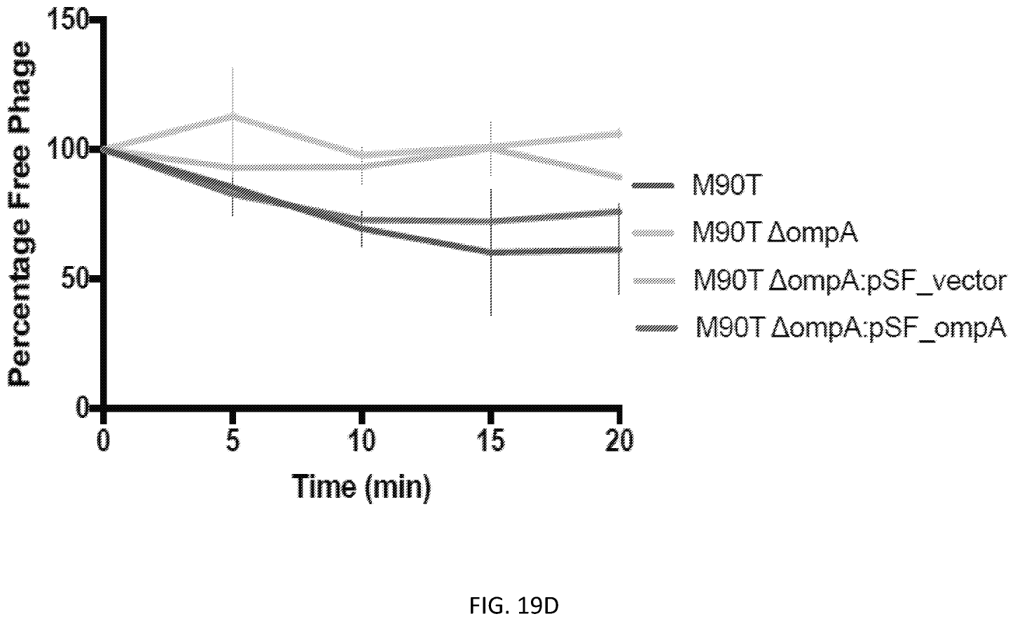

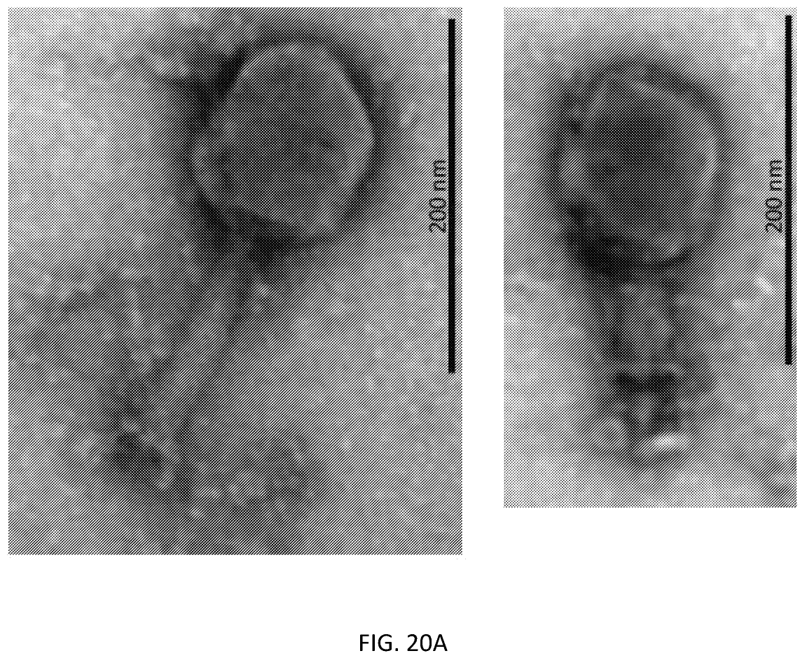

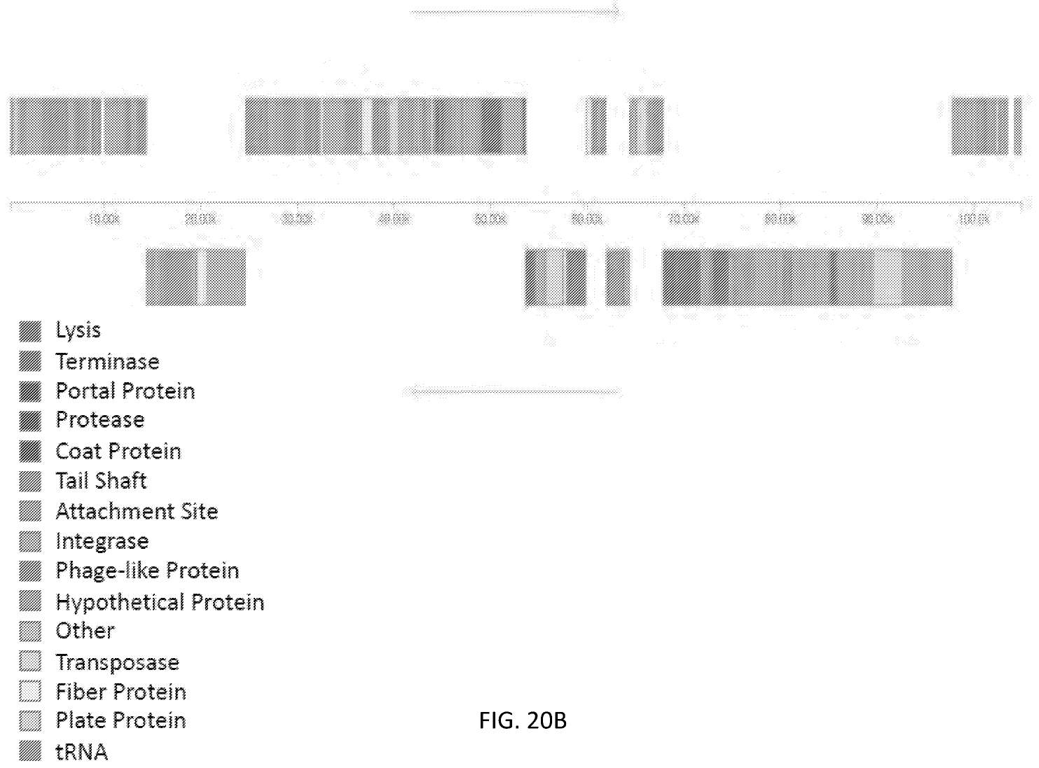

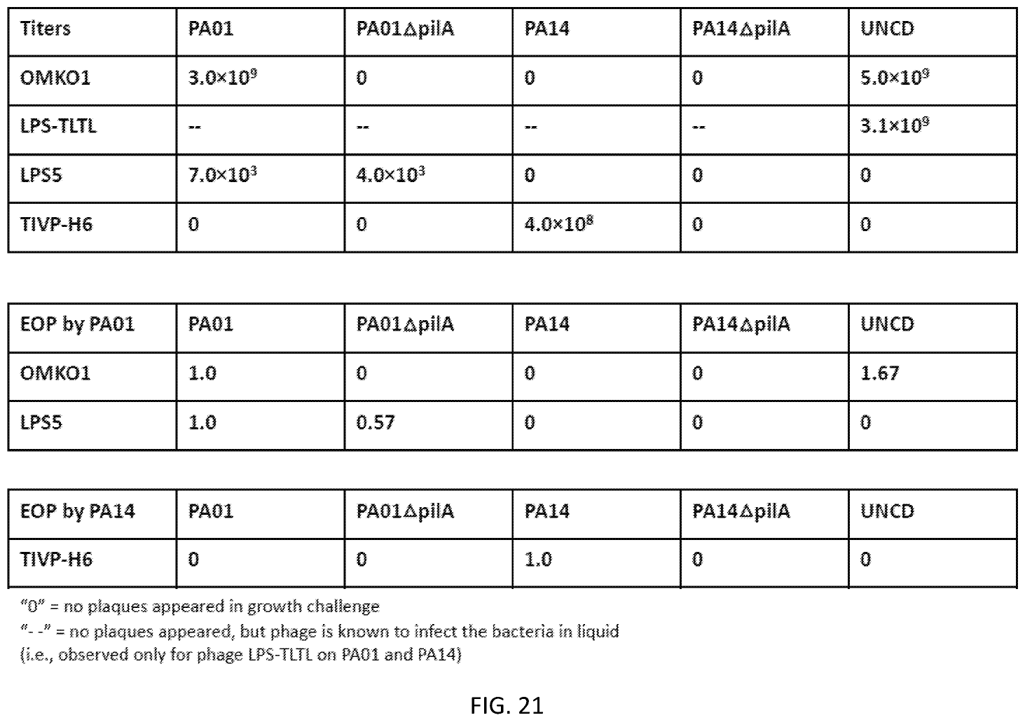

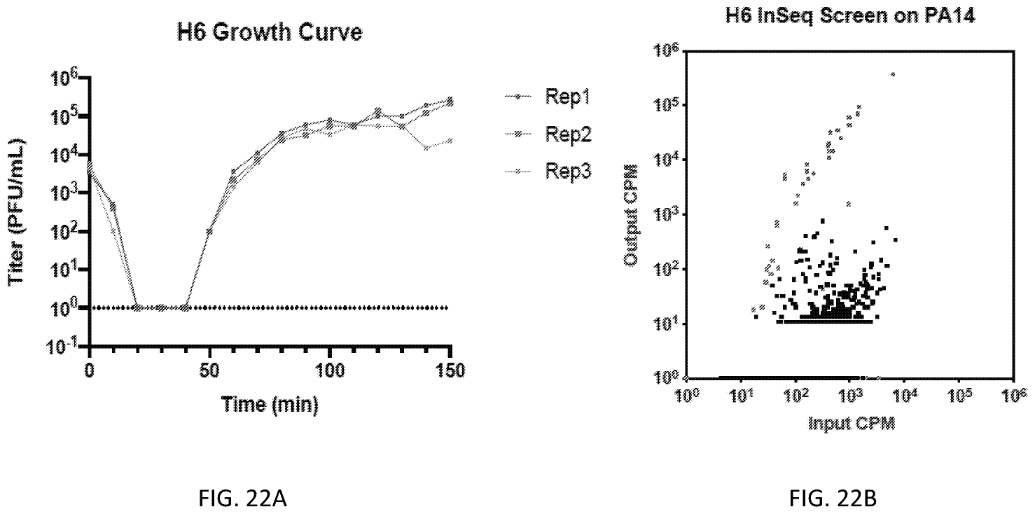

Bacteriophages OMKO1, TIVP-H6, SFA1-1, SF60B, and SF37B were tested to determine if the phage could grow on the target bacterial species and that the phage could not grow on the target species when at least one of multiple putative molecules to which the bacteriophage is intended to bind was knocked out. In each case, the bacteriophage formed plaques on the target bacterium but did not form plaques when the molecule to which it was intended to bind was knocked out (FIGS. 18, 19A-19D, 21, and 22A). Binding of phage TIVP-H6 to P. aeruginosa genes involved in Type IV pilus biogenesis was additionally confirmed by a modified InSeq (insertion sequence) approach, using the methods for InSeq library preparation and InSeq screen described above (FIG. 22B).

Bacteriophages LPS-5 and LPS-TLTL in Table 1 were not tested for binding to putative LPS receptors. Rather, it was observed that spontaneous mutants of the target Gram-negative bacteria (Pseudomonas aeruginosa) with evolved resistance to each of these phages showed classic “rough” colony phenotypes, associated with loss of the O-polysaccharide, the most external LPS moiety (Hancock et al. (1983) Infect. Immun. 42:170-177). Consistency of these observed rough-colony phenotypes led to the conclusion that phages LPS-5 and LPS-TLTL putatively bind to LPS of P. aeruginosa.

The binding target of phage PG-I1 in Table 1 is putatively determined to be peptidoglycan, because of the genetic relatedness of the phage sequence to other phages in the Kayvirus genus that infect S. aureus by using wall techoic acid or other portions of peptidoglycan as the binding receptor.

The binding receptor(s) for bacteriophages SFNHSI and TIVP-27 in Table 1 have not been putatitvely determined at this time using laboratory experiments.

Evolution of resistance in Shigella bacteria to phages SFA1-1, SF37B and SF60B can occur through mutations that alter or delete OmpA and/or OmpC. Shigella bacteria are facultative intracellular pathogens of humans, which replicate in epithelial cells of the colon to cause dysentery disease. Mutational changes in ompA and ompC genes of these bacteria are known to be associated with reduced virulence or loss of virulence in Shigella species, because these proteins abolish intercellular spread of the bacteria. Therefore, selection exerted by phages SFA1-1, SF37B and SF60B can cause Shigella bacteria to evolve a trade-off: evolution of phage resistance causes reduced virulence or avirulence in target bacteria.

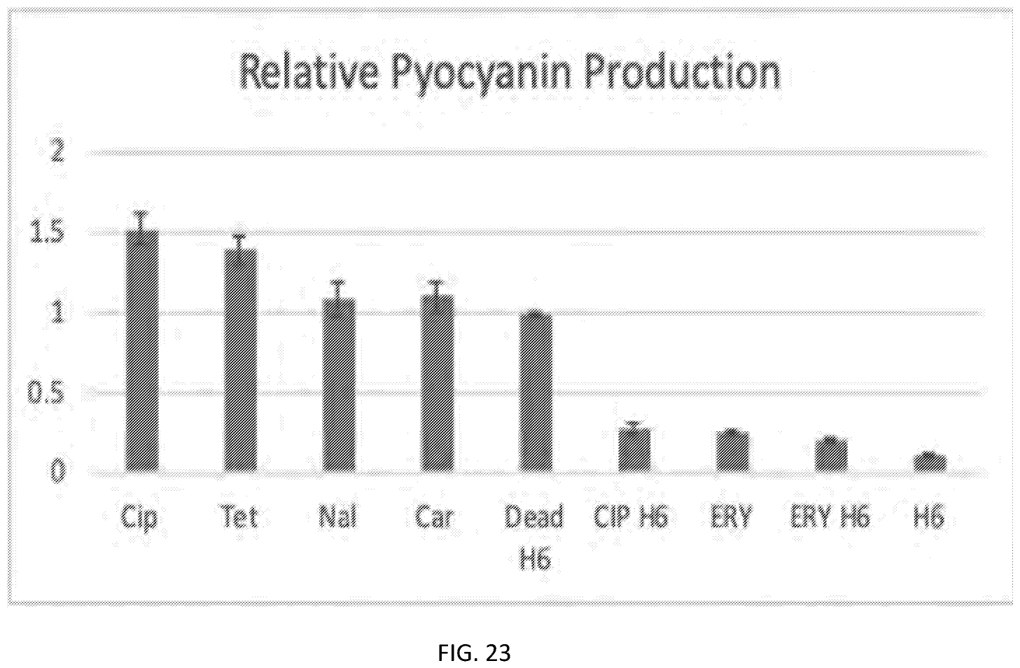

Deletion of type-IV pili is shown to attenuate virulence in P. aeruginosa, by impairing pyocyanin production, twitching motility, and biofilm formation (Persat et al. (2015) Proc. Natl. Acad. Sci. 112:7563-7568). Phage TIVP-H6 selects for phage resistance in P. aeruginosa via bacterial alteration or loss of type-IV pili. Data showed that phage TIVP-H6 caused statistically significantly (P<0.001) reduced pyocyanin production in 48-hour cultures of PA14, similar to controls using sub-inhibitory concentration of erythromycin (Kanoh and Rubin (2010) Clinical Microbiology Reviews 23:590-615) (FIG. 23). Also, the phage did not interfere with effects of erythromycin, and the beneficial phage effects persisted in presence of ciprofloxacin, one of the antibiotics that either increased or did not affect pyocyanin production (FIG. 23).

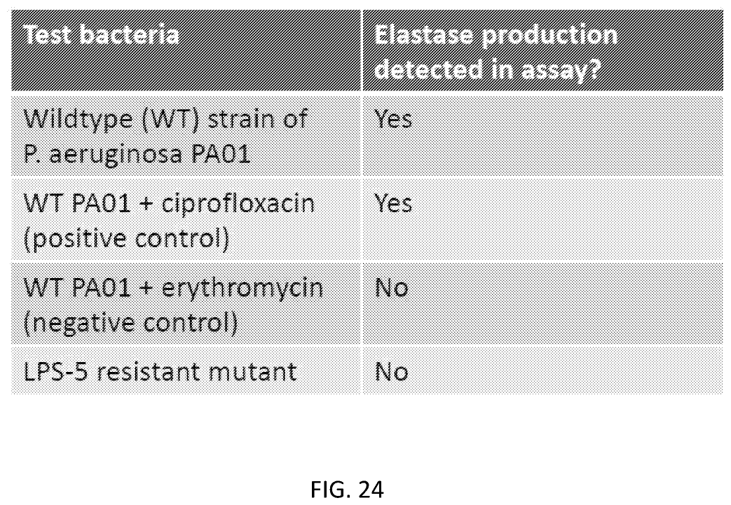

Deletion of O antigen can reduce ability for CF-associated P. aeruginosa strains to produce extracellular elastase (Leone et al. (2008) European Journal of Clinical Microbiology and Infectious Disease 27:1093-1099), and can cause re-sensitivity to hydrophobic antibiotics and impair iron uptake by bacteria (Delcour (2009) Biochimica and Biophysica Acta 1794:808-816). This strongly suggests a mechanism for phage LPS-5 to select for phage resistance in P. aeruginosa via LPS mutations that decrease elastase production and drive antibiotic re-sensitivity. These data used classic Congo-elastase assays (Sachar et al. 1955) Proceedings of the Society for Experimental Biology and Medicine 90:323-326) to confirm that elastase was below the limit of detection in 48-hour-old cultures of phage resistant mutants of PA01 (FIG. 24).

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of increasing antibiotic sensitivity and/or decreasing virulence in a pathogenic bacteria, the method comprising:
contacting the bacteria with a lytic bacteriophage selected from the group consisting of OMKO1, LPS-5, TIVP-H6, LPS-TLTL,TIVP-27, SFA1-1, SF60B, SFNHSI, SF37B, PG-I1, and U136B.

2. The method of claim 1, wherein the bacteria are contacted with bacteriophage at a multiplicity of infection of bacteriophage to bacteria in the range of about 0.05 to about 50.

3. The method of claim 1, wherein the bacteriophage binds at least one molecule selected from the group consisting of an O-antigen, efflux pump, Type IV pilus, LPS (core), OmpA, OmpC, TolC, and peptidoglycan in the bacteria.

4. The method of claim 1, wherein the bacteriophage binds TolC and LPS.

5. The method of claim 1, wherein the bacteriophage binds a protein of a Mex efflux pump.

6. The method of claim 5, wherein the Mex efflux pump is a surface exposed protein.

7. The method of claim 5, wherein the Mex protein is selected from the group consisting of OprM, MexA, MexB, MexX, and MexY.

8. The method of claim 1 further comprising contacting the pathogenic bacteria with an antibiotic.

9. The method of claim 1, wherein the pathogenic bacteria is drug resistant.

10. The method of claim 1, wherein the pathogenic bacteria is a multi-drug resistant (MDR) bacteria.

11. The method of claim 1, wherein the bacteriophage comprises a nucleotide sequence at least 80%, 85%, 90%, 95%, 96%, 97%, 98%, 99%, or 100% identical to any one of SEQ ID NOs: 1-13.

12. The method of claim 1, wherein the bacteriophage is administered to a subject in need thereof.

13. A pharmaceutical composition comprising one or more lytic bacteriophages selected from the group consisting of OMKO1, LPS-5, TIVP-H6, LPS-TLTL, TIVP-27, SFA1-1, SF60B, SFNHSI, SF37B, PG-I1, and U136B.

14. The pharmaceutical composition of claim 13, wherein the bacteriophage binds at least one molecule selected from the group consisting of an O-antigen, Type IV pilus, LPS (core), OmpA, OmpC, TolC, and peptidoglycan on multi-drug resistant (MDR) bacteria.

15. The pharmaceutical composition of claim 13, wherein the one or more bacteriophages comprise a nucleotide sequence at least 80%, 85%, 90%, 95%, 96%, 97% 98%, 99%, or 100% identical to any one of SEQ ID NOs: 1-13.

16. The pharmaceutical composition of claim 13, further comprising an antibiotic.

17. A method of treating a bacterial infection in a subject in need thereof, the method comprising administering the pharmaceutical composition of claim 13 to the subject with the bacterial infection.

18. The method of claim 17, wherein the pharmaceutical composition is administered directly to a site of the bacterial infection.

19. The method of claim 17, further comprising administering an antibiotic to the subject.

20. The method of claim 19, wherein the antibiotic is administered before or after or co-administered with the pharmaceutical composition.

21. The method of claim 17, wherein the bacterial infection is drug resistant.

22. The method of claim 17, wherein the bacterial infection is multi-drug resistant.

23. The method of claim 1, wherein the pathogenic bacteria is associated with a biofilm.

24. The method of claim 1, wherein the pathogenic bacteria is Pseudomonas aeruginosa, a Shigella species, Staphylococcus aureus, or Escherichia coli.

25. The method of claim 24, wherein the pathogenic bacteria is in a biofilm.

26. A method of disrupting a pathogenic bacteria associated with a biofilm, the method comprising contacting the bacteria with a lytic bacteriophage selected from the group consisting of OMKO1, LPS-5, TIVP-H6, LPS-TLTL, TIVP-27, SFA1-1, SF60B, SFNHSI, SF37B, PG-I1, and U136B.

27. A method of preventing formation of a biofilm on a surface, the method comprising contacting the surface with a lytic bacteriophage selected from the group consisting of OMKO1, LPS-5, TIVP-H6, LPS-TLTL, TIVP-27, SFA1-1, SF60B, SFNHSI, SF37B, PG-I1, and U136B.

28. The method of claim 26, wherein the bacteriophage binds a molecule selected from the group consisting of an O-antigen, efflux pump, Type IV pilus, LPS (core), OmpA, OmpC, and TolC in the bacteria and the bacteria either genetically resists bacteriophage infection or becomes infected and lysed by the bacteriophage, and wherein genetically resistant bacteria have impaired efflux pumps and increased sensitivity to one or more antibiotics.

29. The method of claim 26, further comprising contacting the bacteria or surface with the one or more antibiotics.

30. A composition comprising an LPS-5 bacteriophage targeting Pseudomonas aeruginosa, the bacteriophage having a genome comprising a nucleic acid sequence comprising SEQ ID NO: 1.

31. A composition comprising an OMKO1 bacteriophage targeting Pseudomonas aeruginosa, the bacteriophage having a genome comprising a nucleic acid sequence comprising SEQ ID NO: 2.

32. A composition comprising a TIVP-H6 bacteriophage targeting Pseudomonas aeruginosa, the bacteriophage having a genome comprising a nucleic acid sequence comprising SEQ ID NO: 3.

33. A composition comprising an LPS-TLTL bacteriophage targeting Pseudomonas aeruginosa, the bacteriophage having a genome comprising a nucleic acid sequence comprising SEQ ID NO: 4.

34. A composition comprising an TIVP-27 bacteriophage targeting Pseudomonas aeruginosa, the bacteriophage having a genome comprising a nucleic acid sequence comprising SEQ ID NO: 5.

35. A composition comprising an SFA1-1 bacteriophage targeting a Shigella spp. bacteria, the bacteriophage having a genome comprising a nucleic acid sequence comprising SEQ ID NO: 6.

36. A composition comprising an SF60B bacteriophage targeting a Shigella spp. bacteria, the bacteriophage having a genome comprising a nucleic acid sequence comprising SEQ ID NO: 7.

37. A composition comprising an SFNHSI bacteriophage targeting a Shigella spp. bacteria, the bacteriophage having a genome comprising a nucleic acid sequence comprising SEQ ID NO: 8.

38. A composition comprising an SF37B bacteriophage targeting a Shigella spp. bacteria, the bacteriophage having a genome comprising a nucleic acid sequence comprising SEQ ID NO: 9.

39. A composition comprising a PG-I1 bacteriophage targeting Staphylococcus aureus, the bacteriophage having a genome comprising at least one nucleic acid sequence selected from the group consisting of SEQ ID NOs: 10-12.

40. A composition comprising a U136B bacteriophage targeting E. coli, the bacteriophage having a genome comprising a nucleic acid sequence comprising SEQ ID NO: 13.

41. A method of treating a bacterial infection in a subject in need thereof, the method comprising administering to the subject a therapeutically effective amount of a composition according to claim 23.

42. The method of claim 41, wherein the bacterial infection is drug-resistant or multi-drug resistant.
